(19) United States
(12) Patent Application Publication
(10) Pub. No.: US 2026/0285861 A1
(43) Pub. Date: Sep. 24, 2026

(54) Selective Bet Inhibitors and Uses Thereof

(71) Applicant: Tay Therapeutics Limited, Dundee (GB)

(72) Inventors: Christopher Andrew Woodland, Dundee (GB); Mark Bell, Dundee (GB); Iain Stuart, Flanders, NJ (US)

(21) Appl. No.: 19/677,944

(22) Filed: May 14, 2026

Related U.S. Application Data
(63) Continuation of application No. 19/140,608, filed on Dec. 22, 2023.
(60) Provisional application No. 63/520,322, filed on Aug. 17, 2023.
(60) Provisional application No. 63/586,684, filed on Sep. 29, 2023.

(30) Foreign Application Priority Data
Dec. 23, 2022 (GB) 2219706.5
Dec. 28, 2022 (GB) 2219791.7
Feb. 27, 2023 (GB) 2302859.0

(51) Int. Cl. C07D 471/04; A61K 31/444; A61K 31/4545; A61K 31/496; A61K 31/5377; A61P 11/00; A61P 13/12; A61P 17/06; A61P 19/02; A61P 29/00; A61P 37/06

(52) U.S. Cl. CPC C07D 471/04; A61K 31/444; A61K 31/4545; A61K 31/496; A61K 31/5377; A61P 11/00; A61P 13/12; A61P 17/06; A61P 19/02; A61P 29/00; A61P 37/06

(57) ABSTRACT

This disclosure relates to certain pyrrolopyridone compounds. Certain compounds of the disclosure are potent and selective Bromodomain and Extra-Terminal (BET) inhibitors. The disclosure also relates to pharmaceutically acceptable salts of the compounds or N-oxides thereof, methods of treating diseases and disorders using the compounds, salts, or N-oxides thereof and compositions comprising the compounds salts or N-oxides thereof. The disclosure additionally relates to methods for the treatment of inflammatory and autoimmune diseases and disorders (e.g., skin diseases and disorders, joint and joint-related diseases and disorders, and fibrosis or fibrosis-associated diseases or disorders) using potent and selective BET inhibitors and formulations comprising the disclosed BET inhibitors.

### CROSS-REFERENCE TO RELATED APPLICATIONS

This application is a continuation of U.S. application Ser. No. 19/140,608, filed Jun. 18, 2025, which is a U.S. National Stage Application filed under 35 U.S.C. § 371 of International Application No. PCT/US2023/085840, filed Dec. 22, 2023, which claims priority under 35 U.S.C. § 119(e) to U.S. Provisional Application No. 63/586,684, filed Sep. 29, 2023, U.S. Provisional Application No. 63/520,322, filed Aug. 17, 2023, United Kingdom Patent Application No. GB2302859.0, filed Feb. 27, 2023, United Kingdom Patent Application No. GB2219791.7, filed Dec. 28, 2022, and United Kingdom Patent Application No. GB2219706.5, filed Dec. 23, 2022. The contents of these applications are each incorporated herein by reference in their entirety.

This invention relates to compounds comprising a pyrrolopyridone core, and pharmaceutically acceptable salts and compositions of such compounds. The compounds herein are useful as anti-inflammatory and/or other therapies. Therefore, the present disclosure also concerns compounds for use as medicaments, particularly for the treatment of inflammatory diseases.

### BACKGROUND

Diseases and disorders may be multifactorial. They can involve inflammation or can result in inflammation-related disorders. Autoimmune diseases and disorders may result in inflammation or may result in inflammation related disorders. An inflammatory or autoimmune disease or disorder may cause or result in changes, damage and/or wounds. A significant aspect of treatment of many diseases and disorders is to facilitate correct healing. A failed or failing healing process, a poor healing process or an exaggerated healing process may, for example, leave lesions, erosion, wounds, fibrosis and/or other damage.

The present disclosure is directed to methods for the treatment of inflammatory and autoimmune diseases and disorders including e.g., skin diseases and disorders (e.g., psoriasis), arthritic diseases and disorders (e.g., joint related diseases and disorders), and fibrosis or fibrosis-associated diseases or disorders (e.g., pulmonary fibrosis and lupus), using potent and selective Bromodomain and Extra-Terminal (BET) inhibitors, and pharmaceutically acceptable salts thereof, their use for the treatment of diseases or disorders, and compositions/formulations comprising the BET inhibitors (e.g., oral formulations, injections and infusions).

Psoriasis is a skin disease that causes a rash with itchy, scaly patches, most commonly on the knees, elbows, trunk and scalp. Psoriasis is a long-term (chronic) autoimmune condition. It can be painful, interfere with sleep and make it hard to concentrate. There are several types: plaque psoriasis, nail psoriasis, guttate psoriasis, inverse psoriasis, pustular psoriasis, erythrodermic psoriasis and psoriatic psoriasis. Administration of the BET inhibitors disclosed herein their salts and compositions comprising them can a provide a beneficial method of treatment for one, more or all these different types of psoriasis.

Examples of other inflammatory and/or autoimmune skin diseases or disorders that can benefit from administration of the BET inhibitors disclosed herein include pyoderma gangrenosum, generalized pustular psoriasis and palmar plantar pustulosis.

Pyoderma gangrenosum (PG) is a life-threatening, severe autoimmune neutrophilic dermatosis causing significant dermal ulceration with a prevalence in the U.S. of 5.8 per 100,000². The condition predominantly affects adults, but childhood cases are rarely reported. The sex incidence ranges from being equal, to females being predominantly affected in up to 76% of cases. Classical PG presents most commonly as an extremely painful erythematous lesion which rapidly progresses to a blistered or necrotic ulcer. There is often a ragged undermined edge with a violaceous/erythematous border. The lower legs are most frequently affected although PG can present at any site. The lesion may be precipitated by minor trauma. Most cases of PG are of the classic ulcerative type (approximately 85%), but other subtypes include bullous, vegetative, pustular, peristomal and superficial granulomatous variants, with subtypes of PG sometimes transitioning from one form to another. The differential diagnosis includes all other causes of cutaneous ulceration as there are no definitive laboratory or histopathological criteria for PG. The pathogenesis of PG remains unclear however it is recognized that neutrophils play a key role in the disease process. Upregulation of several key proinflammatory and neutrophil chemotactic factors within lesions have been identified and these include IL-1β, IL-17, TNFα, IL-8, IL-6, IL-18, INF-γ, IL-36γ, and IL-23. IL-8 has been demonstrated to produce PG in animal models. IL-8 is also induced in fibroblasts of PG ulcers and its associated ligands are over-expressed in PG.

An important aspect of treatment of PG is wound healing. In many cases, the healing of a wound is imperfect, resulting in the formation of a scar. Attempts to accelerate the healing process may result in elevating the incidence of scar formation. When the wound is bacterially infected, the healing process becomes more challenging and may take longer. Scars are more often caused following improper treatment.

Despite being a well-recognized condition there is often a failure to make an early diagnosis of PG. Potent topical corticosteroids and tacrolimus ointment applied to the ulcer surface are useful and intralesional injections of corticosteroid into the erythematous active border may be considered. In more severe disease, systemic therapy is required. Oral corticosteroids are the mainstay of treatment and are used to gain rapid control. Ciclosporin is used either alone or in combination with corticosteroids as a steroid-sparing agent, in cases where prolonged treatment is required. Other systemic treatments utilized with varying success include colchicine, sulphasalazine, dapsone, minocycline, apremilast and thalidomide.

Some limitations of current therapies include inadequate efficacy of nonsteroidal topical treatments, restrictions on application to particular body regions, “steroid and CNI phobia,” and application site reactions. Potential long-term safety concerns include systemic side-effects and skin atrophy (for striae and other atrophic changes) with topical corticosteroids and increased risk of infections with CNIs.

Generalized Pustular Psoriasis (GPP) is a rare, debilitating, and often life-threatening inflammatory disease characterized by episodic infiltration of neutrophils into the skin, pustule development, and systemic inflammation, which can manifest in the presence or absence of chronic plaque psoriasis. IL-1P, IL-17, IL-8 and IL-36γ are the dominant cytokines increased in GPP. Current treatments are unsatisfactory and warrant a better understanding of GPP pathogenesis.

Palmar Plantar Pustulosis (PPP) is a chronic dermatitis characterized by intra-epidermal vesicles/pustules containing neutrophils (PMN), located on the palms and soles. Although several pathogeneses of PPP such as concomitant tonsillitis, periodontitis or metal allergy have been proposed, the etiology of PPP remains unknown. IL-1, IL-8, IL-17 and IL-36γ are the dominant cytokines increased in PPP. Current treatments are unsatisfactory and warrant a better understanding of PPP pathogenesis.

Many other topical or skin disorders involve inflammation and share similar biomarker patterns and a product which is capable of reducing inflammatory cytokines involved in inflammation and treats or ameliorates the disorder while avoiding or minimizing systemic and skin-related side effects would be advantageous and could improve patient compliance with treatment.

Joint or joint related disorders or diseases are diseases that affect human joints. Arthritis is one example of a well-known joint disease. Osteoarthritis is the most common form of arthritis and involves the wearing away of the cartilage that caps the bones in a person's joints. It is a degenerative joint disease characterized by joint pain and a progressive loss of articular cartilage. Rheumatoid arthritis (RA) is a disease in which the immune system attacks the joints beginning with the lining of the joints. RA is the most frequent autoimmune chronical inflammatory rheumatism, primarily affecting the synovial membrane of multiple joints. Although its etiology is still unknown, it is now acknowledged that during the inflammatory process of arthritis there are three key mediators, the proinflammatory cytokines TNF-α, IL-13 and IL-6 (see Mori et al., Int. Immunol., 23(11):701-712 (2011)).

Regardless of the cause, inflammation of the joints may cause pain, stiffness, swelling, and some redness of the skin about the joint. Steroids (i.e., corticosteroids) are synthetic drugs that are used to treat a variety of inflammatory diseases and conditions. But the administration of corticosteroids, particularly for extended periods of time, can have a number of unwanted side effects or adverse reactions. The effectiveness of corticosteroids generally diminishes with time and there are disadvantages in their use, including a greater susceptibility to infection and peptic ulcers and corticosteroid injection directly into joint tissues may in some subjects worsen joint damage. For example, the unwanted adverse reactions of triamcinolone (which is a corticosteroid) injections include, hypersensitivity reactions, such as anaphylaxis, joint infection and damage, increased risk of infections, alterations in endocrine function, cardiovascular and renal effects, increased intraocular pressure, gastrointestinal perforation, alternations in bone density and behavioral and mood disturbances. As yet another example, the unwanted adverse reactions of dexamethasone (another corticosteroid) include, fluid and electrolyte disturbances, musculoskeletal, gastrointestinal, neurologic, dermatologic, endocrine, ophthalmic, metabolic cardiovascular, anaphylactoid or hypersensitivity reactions, thromboembolism, weight gain, increased appetite, and nausea (see, e.g., Brinks et al., BMC Musculoskelet. Disord., 11:206 (2010)).

Many disorders involve inflammation and share similar biomarker patterns and a product which is capable of reducing inflammatory cytokines involved in inflammation and treats or ameliorates the disorder while avoiding or minimizing side effects or adverse reactions would be advantageous and could improve patient compliance with treatment.

In addition to inflammation, fibrosis can be a problem throughout the body including in organs, such as the kidney, lungs, liver, heart, lymph nodes (e.g., mediastinal fibrosis), bone marrow, skin, tendons, joints, connective tissue, soft tissues, and cavities e.g., retroperitoneal. Fibrosis may be local or systemic.

Defined by the pathological accumulation of extracellular matrix (ECM) proteins, fibrosis results in scarring which may be coupled with thickening of the affected tissue—it is in essence an exaggerated wound healing response which interferes with normal organ function. Fibrosis of the lung is generally characterized by alveolar epithelial cell injury, areas of type 11 cell hyperplasia, accumulation of fibroblasts and myofibroblasts, and the deposition of extracellular matrix proteins. The result is a progressive loss of normal lung architecture and impairment in gas exchange. Accordingly, symptoms can include shortness of breath, a dry cough, feeling tired, weight loss, and nail clubbing (e.g., due to low oxygen in the blood). Fibrosis of the kidney is generally characterized by tubulointerstitial nephritis and/or fibrosis, i.e., inflammation of the kidneys and the deposition of connective tissue in the kidney parenchyma (e.g., collagen fiber deposition), glomerulosclerosis, i.e., scarring of the glomerulus, and nephropathy, i.e., the deterioration of kidney function. The result is the progressive formation of internal scar tissue that leads to end-stage kidney failure. Accordingly, symptoms can include weight loss and poor appetite, edema (i.e., water retention), shortness of breath, fatigue, frequent urination, hematuria, and itchy skin.

Moreover, inflammatory, autoimmune diseases and disorders, and other diseases and disorders that are characterized by fibrosis or scarring, such as Pulmonary Fibrosis (PF), are challenging to treat. As an example, PF is a chronic disease which affects at least 5 million people globally showing aberrant remodeling of lung tissue. PF is part of a larger group of more than 200 interstitial lung diseases (also known as ILDs or diffuse parenchymal lung disease (DPLD)) that are characterized by inflammation and/or scarring in the lung. In ILDs, the injury/damage occurs in the walls of the air sacs (alveoli) of the lung, as well as in the tissue and space around these air sacs (interstitium). When an ILD includes scar tissue in the lung, it is known as PF.

There are many different types of PF that fall into six primary categories. Five are based on the type of induction or exposure, viz: environmental, occupational, drug-induced, radiation-induced, and autoimmune lung disease. The other main category idiopathic pulmonary fibrosis (IPF) is where no cause can be identified. IPF is a chronic, life-threatening, fibrosing lung disease with few treatment options. Patients experience debilitating symptoms, including shortness of breath and difficulty performing daily activities. The current standard of care treatment options for IPF have been shown to have only a modest impact on slowing the progression of the disease and have been associated with significant side effects, leading to poor therapeutic adherence.

Lung damage (scar tissue) caused by PF cannot be repaired and currently approved medications have limited efficacy and suffer from multiple side effects. Medications and therapies can sometimes improve quality of life, help ease symptoms, or slow down the worsening of scarring. Lung transplantation is the only therapeutic option available in severe cases. Supplemental oxygen, pulmonary rehabilitation, and management of symptoms are important treatment options for many types of PF, depending on severity. Additional systemic autoimmune disorders such as lupus can, inter alia, trigger fibrosis. There are four types of lupus. Systemic lupus erythematosus (SLE) is the most common type. SLE is an autoimmune disease in which the immune system attacks its own tissues, causing widespread inflammation and tissue damage, e.g., to the brain, blood vessels, joints, kidneys, lungs, and skin. Lupus that only affects the skin is less common and is called cutaneous lupus erythematosus (CLE). There are three types of CLE—discoid lupus, subacute cutaneous lupus, and acute cutaneous lupus. Neonatal lupus and drug-induced lupus are the least common types of lupus. Lupus can cause serious kidney damage, and kidney failure is one of the leading causes of death among people with lupus. Renal fibrosis has been found in several kidney diseases, including lupus nephritis (LN). LN affects a significant proportion of subjects suffering from SLE. PF is a severe complication of SLE, which could cause poor prognosis in SLE patients. Five main lung problems occur in lupus: pleuritis, acute lupus pneumonitis, chronic (fibrotic) lupus pneumonitis, pulmonary hypertension, and “shrinking lung” syndrome. Another complication of lupus is that many patients will develop a heart abnormality. Lupus may affect the heart pericardium, myocardium, and endocardium as well as the coronary arteries and broader cardiovascular system. Heart problems may be due to cardiac inflammation related directly to lupus, or due to damage to other organs, such as the lungs and renal system. Lupus may cause inflammation of the blood vessels and lead to blood problems, including a reduced number of healthy red blood cells (anemia). Lupus may affect the brain resulting in cognitive disfunction or brain fog. Other complications of lupus include an increased risk of infection, cancer, bone tissue death, and pregnancy complications. Treatment options to date for lupus have been limited and primarily serve to manage symptoms.

Multiple sclerosis (MS) is a debilitating autoimmune disease characterized by central nervous system (CNS) inflammation and demyelination, which can result in permanent damage to and/or deterioration of nerve fibers. Inflammation can be a main trigger leading to CNS damage. The inflammatory process can result in demyelination and also destruction of oligodendrocytes. The damage to nerve fibers results in lesions and/or plaques. Axonal loss and gliosis can occur as a reaction to CNS damage. Because MS is associated with the CNS, symptoms and difficulties associated with MS include visual disturbances, muscle weakness, difficulties with coordination and balance, sensations associated with nerve damage, and cognitive problems and/or decline. There is no cure for MS, and current treatment options have been limited to managing symptoms and aiding in recovery from “attacks” associated with the nerve damage and other symptoms associated with MS.

Bromodomain and Extra-Terminal (BET) proteins are a family of four bromodomain-containing (BRD) proteins (BRD2, BRD3, BRD4 and BRDT). All four members contain two BRDs (located next to each other toward the N-terminal of the proteins) and an extra-terminal domain (Shi et al., Cancer Cell, 25(2):210-225 (2014)). The two BRDs in each BET protein are designated bromodomain I (BDI) and bromodomain II (BDIl). The BIRD is a functional protein domain that contains a defined and predominantly hydrophobic pocket that binds to acetylated lysine residues, typically those found on transcription factors (Shi et al., Cancer Cell, 25(2):210-225 (2014)) or on the N-terminal tails of histone proteins. BRDs function as epigenetic regulators, i.e., they functionally alter gene activity and expression without altering the DNA sequence. For example, BRD4 recruits the transcription factor P-TEFb to promoters leading to altered expression of genes involved in the cell cycle (Yang et al., Mol. Cell Biol., 28:967-976 (2008)). BRD2 and BRD3 also regulate growth promoting genes (Leroy et al., Mol. Cell, 30:51-60 (2008)). Therefore, BRDs are responsible for transducing the signals carried by acetylated lysine residues into various phenotypes. BETs are considered in the art to be ubiquitously expressed in humans except for BRDT, which is normally expressed in the testes but is also expressed by some cancers (Ekaterina et al., Cell J., 19 (Suppl 1):1-8 (2017)).

BET proteins have roles in the regulation of biochemical pathways such as MYC, BCL2, FOSL1, P-TEFb, NFkB, Glucocorticoid signalling and others (Shi et al., Mol. Cell, 54(5):728-36 (2014), Hajmirza et al., Biomedicines, 6 (1):E16 (2018), Shan et al., Elife, Sep. 11 (6):e27861 (2017), Huang et al., Mol. Cell. Biol., 29(5):1375-87 (2009)). As such, BET inhibitors are considered to have potential uses in a range of inflammatory diseases, cancers, infections, metabolic diseases, CNS disorders, fibrotic diseases, and cardiac diseases (Deanna et al., J Exp Med. 210(11):2181-2190 (2013), Rab et al., Trends Pharmacol. Sci., 33(3):146-53 (2012), Anna et al., J. Immunol., 190(7):3670-3678 (2013), Zuber et al., Nature, 478(7370):524-28. (2011), Montserrat et al., Epigenetics, 12(5):323-339 (2017), Qiming et al., Sci. Transl. Med., 9 (390):eaah5084 (2017), Kristin et al., J. Biol. Chem., 292 (32):13284-13295 (2017), Ning et al., PNAS, 112 (51):15713-18 (2015).

BET proteins ‘read’ acetylated lysines, enable chromatin remodeling and recruit transcription factors. In other words, BRDs are responsible for transducing the signals carried by acetylated lysine residues into various phenotypes and play a key role in regulating gene transcription via epigenetic interactions (“reading”) between the bromodomains and acetylated histones during cell proliferation and differentiation. For example, BRD4 recruits the transcription factor P-TEFb to promoters leading to altered expression of genes involved in the cell cycle.

The two bromodomains (BD1 and BD2) of BET proteins play different roles in regulating gene expression. BD1 regulates housekeeping gene activity, including the regulation of steady-state genes. Notably, BD1 has also been found to be associated with gastrointestinal and hematological toxicity, whereas BD2 has not. On the other hand, BD2 regulates induction of inflammatory genes, and specifically inflammatory genes associated with inflammatory diseases and disorders, including autoimmune diseases. The inhibition of BDII domain of BET proteins has been shown to effect inflammatory diseases, metabolic disease, cancers, and fibrotic diseases (Gilan et al., Science, 368:387-394 (2020), Tsujikawa et al., Clin. Epigenetics, 11(1):102 (2019), Faivre et al., Nature, 578:306-310 (2020), Zhang et al., Cell. Signal., 61:20-29 (2019)).

Compounds that can inhibit or affect the function of BET proteins have the potential to modulate gene expression and treat diseases that are at least in part caused by abnormal regulation of BET protein activity.

A product that is safe, well-tolerated, and prevents occurrence and/or reduces the grade of severity of the incidences, for example, of a joint or joint related disorders or diseases, while avoiding unwanted side effects and adverse reactions would be advantageous and could improve patient compliance with treatment. Accordingly, there is a medical need to replace corticosteroids with safer and better drugs in order to reduce the systemic side effects associated with the administration of corticosteroids. In addition, there is a medical need to slow, arrest, reverse, or otherwise inhibit structural damage to tissues caused by inflammatory diseases, such as damage to articular tissues resulting from, for example, osteoarthritis or rheumatoid arthritis. Involvement of bursas, tendons, and tendon sheaths can be part of arthritic disease. Similarly, there is a medical need to slow, arrest, reverse, or otherwise inhibit fibrosis and the negative consequences thereof including reduced organ function and ultimately failure. Compounds that can inhibit or affect the function of BET proteins have the potential to modulate gene expression and treat diseases that are at least in part caused by abnormal regulation of BET protein activity.

The present disclosure provides novel, potent and selective BET protein inhibitors, their use as medicaments, compositions containing them and processes for their preparation.

### BRIEF SUMMARY OF THE DISCLOSURE

The present disclosure provides potent and selective BET protein inhibitors, their use as medicaments, compositions containing them, and processes for their preparation.

In some embodiments, the present disclosure provides a compound of formula (1), or a pharmaceutically acceptable salt or N-oxide thereof:

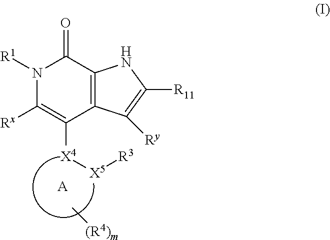

wherein:
Ring A is independently selected from phenyl, 5-membered heterocyclyl, and 6-membered heterocyclyl, wherein X⁴ and X⁵ are independently selected from carbon and nitrogen;
R¹ is independently selected from C₁-C₅-alkyl, C₁-C₅-haloalkyl, C₂-C₆-alkynyl, COR⁶, CO₂R⁶, C₁-C₄-alkylene-NR⁵R⁶, C₁-C₄-alkylene-OR⁷, C₁-C₄-alkyl-S(O)₂R⁶, C₃-C₆-cycloalkyl, aryl, heteroaryl, and 3- to 6-membered heterocycloalkyl;
R² is absent or independently selected from H, halo, cyano, nitro, SF₄, SF₅, ═O, S(O)₂R⁶, alkoxy, C₁-C₆-haloalkyl, C₁-C₈-alkyl, C₃-C₆-cycloalkyl, 4- to 8-membered heterocycloalkenyl, 3- to 8-membered heterocycloalkyl, aryl, and 5- or 6-membered heteroaryl,
wherein the C₁-C₈-alkyl, alkoxy, C₃-C₆-cycloalkyl, 4- to 8-membered heterocycloalkenyl, 3- to 8-membered heterocycloalkyl, aryl, and 5- or 6-membered heteroaryl are independently optionally substituted with one or more groups selected from halogen, cyano, nitro, hydroxy, SF₅, amide, ester, alkoxy, and C₁-C₄-alkyl;
R³ is independently selected from R^{3a} and OR^{3b};
R^{3a} is independently selected from H, CN, C₁-C₄-alkyl, C₂-C₄-alkenyl, C₂-C₄-alkynyl, C₁-C₄-haloalkyl, C₂-C₄-haloalkenyl, C₃-C₈-cycloalkyl, C₅-C₈-cycloalkenyl, 5- to 9-membered heterocycloalkenyl, 3- to 9-membered heterocycloalkyl, phenyl, and 5- to 9-membered heteroaryl;
wherein the cycloalkyl, heterocycloalkyl, cycloalkenyl, and heterocycloalkenyl are independently optionally substituted with from 1 to 4 R⁸ groups; and
wherein the phenyl and the 5- to 9-membered heteroaryl are independently optionally substituted with from 1 to 5 R⁹ groups;
R^{3b} is independently selected from C₁-C₄-alkyl, C₂-C₄-alkylene-O—C₁-C₄-alkyl, C₁-C₄-haloalkyl, C₃-C₈-cycloalkyl, 3- to 8-membered heterocycloalkyl, phenyl, and 5- or 6-membered heteroaryl;
wherein the cycloalkyl and 3- to 8-membered heterocycloalkyl are independently optionally substituted with from 1 to 4 R⁸ groups; and
wherein the phenyl and the 5- or 6-membered heteroaryl are independently optionally substituted with from 1 to 5 R⁹ groups;
R⁴ is independently at each occurrence selected from H, ═O, ═S, halo, nitro, cyano, C₀-C₄-alkylene-NR⁵R⁶, —NR⁵R⁶, C₀-C₄-alkylene-OR⁷, —OR⁷, SR⁶, SOR⁶, C₀-C₄-alkylene-S(O)₂R⁶, —S(O)₂R⁶, SO₂NR⁶R⁶, C₀-C₄-alkylene-CO₂R⁶, —CO₂R⁶, C₀-C₄-alkylene-C(O)R⁶, —C(O)R⁶, C₀-C₄-alkylene-CONR⁶R⁶, —CONR⁶R⁶, C₁-C₆-alkyl, C₁-C₄-alkyl-S(O)₂R⁶, C₂-C₄-alkenyl, C₂-C₄-alkynyl, C₁-C₆-haloalkyl, C₃-C₆-cycloalkyl, aryl, 4- to 6-membered heterocycloalkyl, and 5- or 6-membered heteroaryl;
wherein the aryl, 4- to 6-membered heterocycloalkyl, and 5- or 6-membered heteroaryl are independently optionally substituted with from 1 to 5 R⁹ groups;
R⁵ is independently at each occurrence selected from H, C₁-C₄-alkyl, C(O)—C₁-C₄-alkyl, and S(O)₂—C₁-C₄-alkyl; or R⁵ and R⁶, together with the nitrogen atom to which they are attached, form a C₅-C₈-heterocycloalkyl group optionally substituted with from 1 to 4 R⁸ groups;
R⁶ is independently at each occurrence selected from H and C₁-C₆-alkyl; or where two R⁶ groups are attached to the same nitrogen, those two R⁶ groups, together with the nitrogen atom to which they are attached, optionally form a C₅-C₃-heterocycloalkyl group optionally substituted with from 1 to 4 R⁸ groups;
R⁷ is independently at each occurrence selected from H, C₁-C₄-alkyl, C(O)—C₁-C₄-alkyl, and C₁-C₄-haloalkyl;
R⁸ is independently at each occurrence selected from ═O, ═S, fluoro, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl, 4- to 6-membered heterocycloalkyl, and C₃-C₆-cycloalkyl;
R⁹ is independently at each occurrence selected from halo, nitro, cyano, NRSRs, C₁-C₄-alkyl-OR⁷, OR⁷, SR⁶, SOR⁶, C₁-C₄-alkyl-S(O)₂R⁶, S(O)₂R⁶, SO₂NR⁶R⁶, CO₂R⁶, C(O)R⁶, CONR⁶R⁶, C₀-C₄-alkylene-R^{9a}, C₁-C₄-alkyl, C₂-C₄-alkenyl, C₂-C₄-alkynyl, C₁-C₄-haloalkyl, 4- to 6-membered heterocycloalkyl, and C₃-C₈-cycloalkyl; or where two R⁹ groups are attached to adjacent atoms, those two R⁹ groups, together with the atoms to which they are attached, optionally form a 5- or 6-membered heterocycloalkyl group optionally substituted with from 1 to 4 R⁸ groups;
R^{9a} is independently at each occurrence selected from 4- to 6-membered heterocycloalkyl;
R^{1O} is absent or is independently at each occurrence selected from H, halo, C₁-C₆-alkyl, C₁-C₆-haloalkyl, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₆-alkyl, C₂-C₆-alkenyl, C₂-C₆-alkynyl and 3- to 8-membered heterocycloalkyl;
R¹¹ is independently selected from phenyl, heteroaryl, and heterocyclyl, each optionally substituted with 1 to 4 R² groups and 1 to 3 R¹⁰ groups;
R^{x} and R^{y} are each independently selected from H, halo, nitro, cyano, C₁-C₆-alkylene-NR⁵R⁶, NR⁵R⁶, C₁-C₆-alkylene-OR⁷, C₁-C₆-alkyl-OR⁷, OR⁷, C₁-C₆-alkyl-SR⁶, SR⁶, C₁-C₆-alkyl-SOR⁶, SOR⁶, C₁-C₆-alkyl-S(O)₂R⁶, S(O)₂R⁶, C₁-C₆-alkyl-SO₂NR⁶R⁶, SO₂NR⁶R⁶, C₁-C₆-alkyl-CO₂R⁶, CO₂R⁶, C₁-C₆-alkyl-C(O)R⁶, C(O)R⁶, C₁-C₆-alkyl-CONR⁶R⁶, CONR⁶R⁶, C₁-C₆-alkyl, C₂-C₆-alkenyl, C₂-C₆-alkynyl, C₁-C₆-haloalkyl, C₃-C₈-cycloalkyl, 3- to 8-membered heterocycloalkyl, aryl, aryloxy, and 5- to 8-membered heteroaryl;
m is an integer selected from 0, 1, 2, 3 and 4;
wherein any of the aforementioned alkyl, alkylene, alkenyl, or C₃-C₆-cycloalkyl groups is optionally substituted, where chemically possible, by 1 to 5 substituents which are each independently at each occurrence selected from the group consisting of: C₁-C₄-alkyl, oxo, fluoro, nitro, cyano, NR^{a}R^{b}, OR^{a}, SR^{a}, CO₂R^{a}, C(O)R^{a}, CONR^{a}R^{a}, S(O)R^{a} and S(O)₂R^{a};
wherein R^{a} is independently at each occurrence selected from H, C₁-C₄-alkyl and C₁-C₄-haloalkyl; and
wherein R^{b} is independently at each occurrence selected from H, C₁-C₄-alkyl, C(O)—C₁-C₄-alkyl and S(O)₂—C₁-C₄-alkyl.

In some embodiments, the present disclosure provides a compound of formula (IA), or a pharmaceutically acceptable salt or N-oxide thereof:

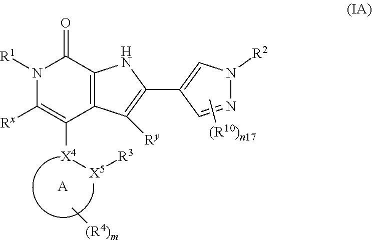

wherein:
Ring A is independently selected from phenyl, 5-membered heterocyclyl and 6-membered heterocyclyl, wherein X⁴ is independently selected from carbon and nitrogen and X⁵ is independently selected from carbon and nitrogen;
R¹ is independently selected from C₁-C₃-alkyl, C₁-C₃-fluoroalkyl, C₃-C₄-cycloalkyl and 4-membered heterocycloalkyl;
R² is independently selected from C₁-C₄-haloalkyl, ethyl, cyano, nitro, isopropyl, tert-butyl, cyclopropyl, and SF₅;
R³ is independently selected from R^{3a}, OR^{3b}, and NR⁶R^{3b};
R^{3a} is independently selected from H, CN, C₁-C₄-alkyl, C₂-C₄-alkenyl, C₂-C₄-alkynyl, C₁-C₄-haloalkyl, C₂-C₄-haloalkenyl, and C₀-C₃-alkylene-R^{3c}; wherein R^{3c} is independently at each occurrence selected from C₃-C₃-cycloalkyl, C₅-C₈-cycloalkenyl, 5- to 8-membered heterocycloalkenyl, 3- to 8-membered heterocycloalkyl, phenyl and 5- or 6-membered heteroaryl; wherein where R^{3c} is cycloalkyl, heterocycloalkyl, cycloalkenyl, or heterocycloalkenyl, R^{3c} is optionally substituted with from 1 to 4 R⁸ groups and where R^{3c} is phenyl or heteroaryl, R^{3c} is optionally substituted with from 1 to 5 R⁹ groups;
R^{3b} is independently selected from C₁-C₄-alkyl, C₂-C₄-alkylene-O—C₁-C₄-alkyl, C₁-C₄-haloalkyl and C₀-C₃-alkylene-R^{3d}; wherein R^{3d} is independently at each occurrence selected from C₃-C₈-cycloalkyl, 3- to 8-membered heterocycloalkyl, phenyl and 5- or 6-membered heteroaryl; wherein where R^{3d} is cycloalkyl or heterocycloalkyl, R^{3d} is optionally substituted with from 1 to 4 R⁸ groups and where R^{3d} is phenyl or heteroaryl, R^{3d} is optionally substituted with from 1 to 5 R⁹ groups;
R⁴ is independently at each occurrence selected from ═O, ═S, halo, nitro, cyano, C₀-C₄-alkylene-NR⁵R⁶, C₀-C₄-alkylene-OR⁷, SRS, SOR⁶, C₀-C₄-alkylene-S(O)₂R⁶, SO₂NR⁶R⁶, C₀-C₄-alkylene-CO₂R⁶, C₀-C₄-alkylene-C(O)R⁶, C₀-C₄-alkylene-CONR⁶R⁶, C₁-C₄-alkyl, C₂-C₄-alkenyl, C₂-C₄-alkynyl, C₁-C₄-haloalkyl, C₃-C₆-cycloalkyl, and C₀-C₄-alkylene-R^{4c};
R^{4c} is independently at each occurrence selected from C₃-C₆-cycloalkyl and 4- to 6-membered heterocycloalkyl;
R⁵ is independently at each occurrence selected from H, C₁-C₄-alkyl, C(O)—C₁-C₄-alkyl and S(O)₂—C₁-C₄-alkyl; or R⁵ and R⁶, together with the nitrogen atom to which they are attached form a C₅-C₈-heterocycloalkyl group optionally substituted with from 1 to 4 R⁸ groups:
R⁶ is independently at each occurrence selected from H and C₁-C₄-alkyl; or where two R⁶ groups are attached to the same nitrogen, those two R⁶ groups together with the nitrogen atom to which they are attached optionally form a C₅-C₈-heterocycloalkyl group optionally substituted with from 1 to 4 R⁸ groups;
R⁷ is independently at each occurrence selected from H, C₁-C₄-alkyl, C(O)—C₁-C₄-alkyl and C₁-C₄-haloalkyl;
R⁸ is independently at each occurrence selected from ═O, ═S, fluoro, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl and cyclopropyl;
R⁹ is independently at each occurrence selected from halo, nitro, cyano, C₀-C₄-alkylene-NR⁵R⁶, C₀-C₄-alkylene-OR^{7a}, C₀-C₄-alkylene-SR⁶, C₀-C₄-alkylene-SOR⁶, C₀-C₄-alkylene-S(O)₂R⁶, C₀-C₄-alkylene-SO₂N⁶R⁶R⁶, C₀-C₄-alkylene-C₂R⁶, C₀-C₄-alkylene-C(O)R⁶, C₀-C₄-alkylene-CONR⁶R⁶, C₀-C₄-alkylene-R^{9a}, C₁-C₄-alkyl, C₂-C₄-alkenyl, C₂-C₄-alkynyl and C₁-C₄-haloalkyl; or where two R⁹ groups are attached to adjacent atoms, those two R⁹ groups together with the atoms to which they are attached optionally form a 5- or 6-membered heterocycloalkyl group optionally substituted with from 1 to 4 R⁶ groups;
R^{7a} is independently at each occurrence selected from H, C₁-C₄-alkyl, C(O)—C₁-C₄-alkyl, C₀-C₄-alkylene-NR₅R₆, —C₀-C₄-alkyl-O—R⁷, C₀-C₄-alkylene-SR⁶, C₀-C₄-alkylene-SOR⁶, C₀-C₄-alkylene-S(O)₂R⁶, C₀-C₄-alkylene-SO₂NR⁶R⁶, C₀-C₄-alkylene-CO₂R⁶, C₀-C₄-alkylene-C(O)R⁶, C₀-C₄-alkylene-CONR⁶R⁶ and C₁-C₄-haloalkyl;
R^{9a} is independently at each occurrence selected from C₃-C₆-cycloalkyl and 4- to 6-membered heterocycloalkyl;
R¹⁰ is independently at each occurrence selected from halo, C₁-C₄-alkyl, C₁-C₄-haloalkyl, nitro, cyano, NR⁵R⁶, OR⁷, SIR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl and 4-membered heterocycloalkyl;
Rₓ and R_{y} are each independently selected from H, halo, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl, C₃-C₄-cycloalkyl and 4-membered heterocycloalkyl;
m is an integer selected from 0, 1, 2, 3 and 4;
n¹⁷ is an integer selected from 0, 1 and 2;
wherein any of the aforementioned alkyl, alkylene, alkenyl, cycloalkyl or heterocycloalkyl groups is optionally substituted, where chemically possible, by 1 to 5 substituents which are each independently at each occurrence selected from the group consisting of: C₁-C₄-alkyl, oxo, fluoro, nitro, cyano, NR^{a}R^{b}, OR^{a}, SR^{a}, CO₂R^{a}, C(O)R^{a}, CONR^{a}R^{a}, S(O)R^{a} and S(O)₂R^{a}; wherein R^{a} is independently at each occurrence selected from H, C₁-C₄-alkyl and C₁-C₄-haloalkyl; and R^{b} is independently at each occurrence selected from H, C₁-C₄-alkyl, C(O)—C₁-C₄-alkyl and S(O)₂—C₁-C₄-alkyl.

In some embodiments, the present disclosure provides a compound of formula (XI), or a pharmaceutically acceptable salt or N-oxide thereof:

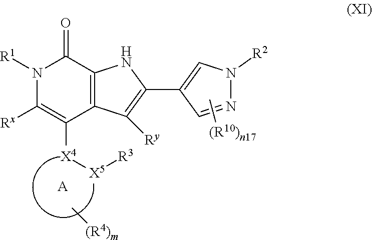

wherein:
Ring A is independently selected from phenyl, 5-membered heterocyclyl and 6-membered heterocyclyl, wherein X⁴ is independently selected from carbon and nitrogen and X⁵ is independently selected from carbon and nitrogen;
R¹ is independently selected from C₁-C₃-alkyl, C₁-C₃-fluoroalkyl, C₃-C₄-cycloalkyl and 4-membered heterocycloalkyl;
R² is independently selected from C₁-C₄-haloalkyl, ethyl, cyano, nitro, isopropyl, tert-butyl, cyclopropyl, and SF₅;
R³ is independently selected from R^{3a}, OR^{3b}, and NR⁶R^{3b};
R^{3a} is independently selected from H, CN, C₁-C₄-alkyl, C₂-C₄-alkenyl, C₂-C₄-alkynyl, C₁-C₄-haloalkyl, C₂-C₄-haloalkenyl, and C₀-C₃-alkylene-R^{3c}; wherein R^{3c} is independently at each occurrence selected from C₃-C₈-cycloalkyl, C₅-C₈-cycloalkenyl, 5- to 8-membered heterocycloalkenyl, 3- to 8-membered heterocycloalkyl, phenyl and 5- or 6-membered heteroaryl; wherein where R^{3c} is cycloalkyl, heterocycloalkyl, cycloalkenyl, or heterocycloalkenyl, R^{3c} is optionally substituted with from 1 to 4 R⁸ groups and where R^{3c} is phenyl or heteroaryl, R^{3c} is optionally substituted with from 1 to 5 R⁹ groups;
R^{3b} is independently selected from C₁-C₄-alkyl, C₂-C₄-alkylene-O—C₁-C₄-alkyl, C₁-C₄-haloalkyl and C₀-C₃-alkylene-R^{3d}; wherein R^{3d} is independently at each occurrence selected from C₃-C₈-cycloalkyl, 3- to 8-membered heterocycloalkyl, phenyl and 5- or 6-membered heteroaryl; wherein where R^{3d} is cycloalkyl or heterocycloalkyl, R^{3d} is optionally substituted with from 1 to 4 R⁸ groups and where R^{3d} is phenyl or heteroaryl, R^{3d} is optionally substituted with from 1 to 5 R⁹ groups;
R⁴ is independently at each occurrence selected from ═O, ═S, halo, nitro, cyano, C₀-C₄-alkylene-NR⁵R⁶, C₀-C₄-alkylene-OR⁷, SR⁶, SOR⁶, C₀-C₄-alkylene-S(O)₂R⁶, SO₂NR⁶R⁶, C₀-C₄-alkylene-CO₂R⁶, C₀-C₄-alkylene-C(O)R⁶, C₀-C₄-alkylene-CONR⁶R⁶, C₁-C₄-alkyl, C₂-C₄-alkenyl, C₂-C₄-alkynyl, C₁-C₄-haloalkyl, C₃-C₆-cycloalkyl, and C₀-C₄-alkylene-R^{4c};
R^{4c} is independently at each occurrence selected from C₃-C₆-cycloalkyl and 4- to 6-membered heterocycloalkyl;
R⁵ is independently at each occurrence selected from H, C₁-C₄-alkyl, C(O)—C₁-C₄-alkyl and S(O)₂—C₁-C₄-alkyl; or R⁵ and R⁶, together with the nitrogen atom to which they are attached form a C₅-C₈-heterocycloalkyl group optionally substituted with from 1 to 4 R⁸ groups;
R⁶ is independently at each occurrence selected from H and C₁-C₄-alkyl; or where two R⁶ groups are attached to the same nitrogen, those two R⁶ groups together with the nitrogen atom to which they are attached optionally form a C₅-C₈-heterocycloalkyl group optionally substituted with from 1 to 4 R⁸ groups;
R⁷ is independently at each occurrence selected from H, C₁-C₄-alkyl, C(O)—C₁-C₄-alkyl and C₁-C₄-haloalkyl;
R⁸ is independently at each occurrence selected from ═O, ═S, fluoro, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl and cyclopropyl;
R⁹ is independently at each occurrence selected from halo, nitro, cyano, C₀-C₄-alkylene-NR⁵R⁶, C₀-C₄-alkylene-OR^{7a}, C₀-C₄-alkylene-SR⁶, C₀-C₄-alkylene-SOR⁶, C₀-C₄-alkylene-S(O)₂R⁶, C₀-C₄-alkylene-SO₂NR⁶R⁶, C₀-C₄-alkylene-CO₂R⁶, C₀-C₄-alkylene-C(O)R⁶, C₀-C₄-alkylene-CONR⁶R⁶, C₀-C₄-alkylene-R^{9a}, C₁-C₄-alkyl, C₂-C₄-alkenyl, C₂-C₄-alkynyl, C₁-C₄-haloalkyl; or where two R⁹ groups are attached to adjacent atoms, those two R⁹ groups together with the atoms to which they are attached optionally form a 5- or 6-membered heterocycloalkyl group optionally substituted with from 1 to 4 R⁸ groups;
R^{7a} is independently at each occurrence selected from H, C₁-C₄-alkyl, C(O)—C₁-C₄-alkyl, C₀-C₄-alkylene-NR₅R₆, —C₀-C₄-alkyl-O—R⁷, C₀-C₄-alkylene-SR⁶, C₀-C₄-alkylene-SOR⁶, C₀-C₄-alkylene-S(O)₂R⁶, C₀-C₄-alkylene-SO₂NR⁶R⁶, C₀-C₄-alkylene-C₂R⁶, C₀-C₄-alkylene-C(O)R⁶, C₀-C₄-alkylene-CONR⁶R⁶ and C₁-C₄-haloalkyl;
R^{9a} is independently at each occurrence selected from C₃-C₆-cycloalkyl and 4- to 6-membered heterocycloalkyl;
R¹⁰ is independently at each occurrence selected from halo, C₁-C₄-alkyl, C₁-C₄-haloalkyl, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl and 4-membered heterocycloalkyl;
R^{x} and R^{y} are each independently selected from H, halo, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl, C₃-C₄-cycloalkyl and 4-membered heterocycloalkyl;
m is an integer selected from 0, 1, 2, 3 and 4;
n₁₇ is an integer selected from 0, 1 and 2;
wherein any of the aforementioned alkyl, alkylene, alkenyl, cycloalkyl or heterocycloalkyl groups is optionally substituted, where chemically possible, by 1 to 5 substituents which are each independently at each occurrence selected from the group consisting of: C₁-C₄-alkyl, oxo, fluoro, nitro, cyano, NR^{a}R^{b}, OR^{a}, SR^{a}, CO₂R^{a}, C(O)R^{a}, CONR^{a}R^{a}, S(O)R^{a} and S(O)₂R^{a}; wherein R^{a} is independently at each occurrence selected from H, C₁-C₄-alkyl and C₁-C₄-haloalkyl; and R^{b} is independently at each occurrence selected from H, C₁-C₄-alkyl, C(O)—C₁-C₄-alkyl and S(O)₂—C₁-C₄-alkyl.

In some embodiments, R² is C₁-C₄-haloalkyl.

In some embodiments, R^{x} is H.

In some embodiments, X⁴ is carbon.

In some embodiments, R¹ is selected from methyl and ethyl.

In some embodiments, R² is CF₃.

In some embodiments, n₁₇ is 0.

In some embodiments, Ring A is pyridone.

In some embodiments, Ring A is substituted on the nitrogen with 1 group selected from H, C₁-C₄-alkyl, cyclopropyl, cyclobutyl, methyl-cyclobutyl and 4-membered heterocycloalkyl.

In some embodiments, Ring A is

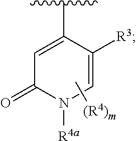

wherein R^{4a} is selected from H, C₁-C₄-alkyl, cyclopropyl and 4-membered heterocycloalkyl.

In some embodiments, R^{4a} is selected from methyl, cyclopropyl, oxetane, —CH₂—CH₂—OMe and azetidine.

In some embodiments, R³ is R³.

In some embodiments, R^{3a} is phenyl optionally substituted with from 1 to 3 R⁹ groups.

In some embodiments, R^{y} is H.

In some embodiments, R^{y} is halo.

In some embodiments, the present disclosure provides a compound selected from:

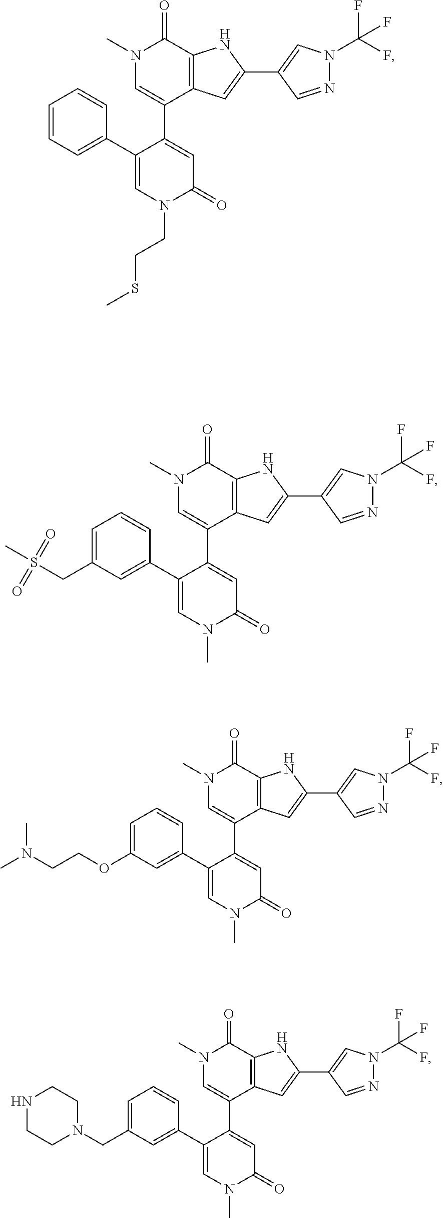 [SMILES: CSCCn1cc(-c2ccccc2)c(-c2cn(C)c(=O)c3[nH]c(-c4cnn(C(F)(F)F)c4)cc23)cc1=O ; Cn1cc(-c2cccc(CS(C)(=O)=O)c2)c(-c2cn(C)c(=O)c3[nH]c(-c4cnn(C(F)(F)F)c4)cc23)cc1=O ; CN(C)CCOc1cccc(-c2cn(C)c(=O)cc2-c2cn(C)c(=O)c3[nH]c(-c4cnn(C(F)(F)F)c4)cc23)c1 ; Cn1cc(-c2cccc(CN3CCNCC3)c2)c(-c2cn(C)c(=O)c3[nH]c(-c4cnn(C(F)(F)F)c4)cc23)cc1=O]

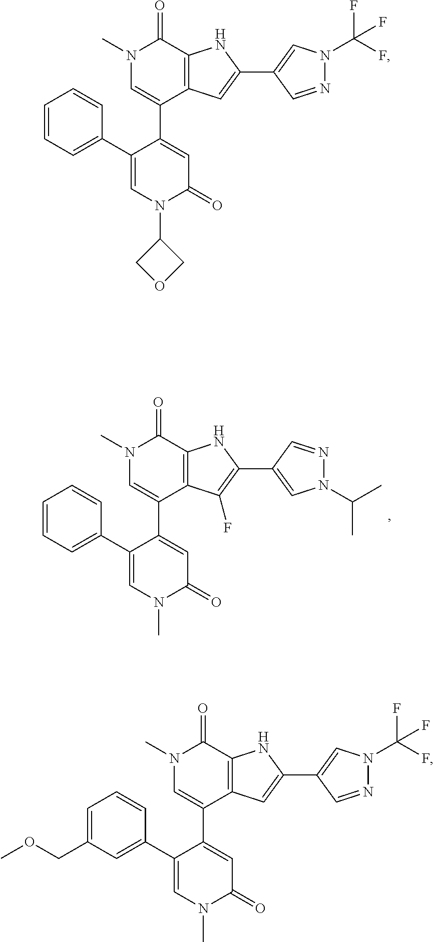

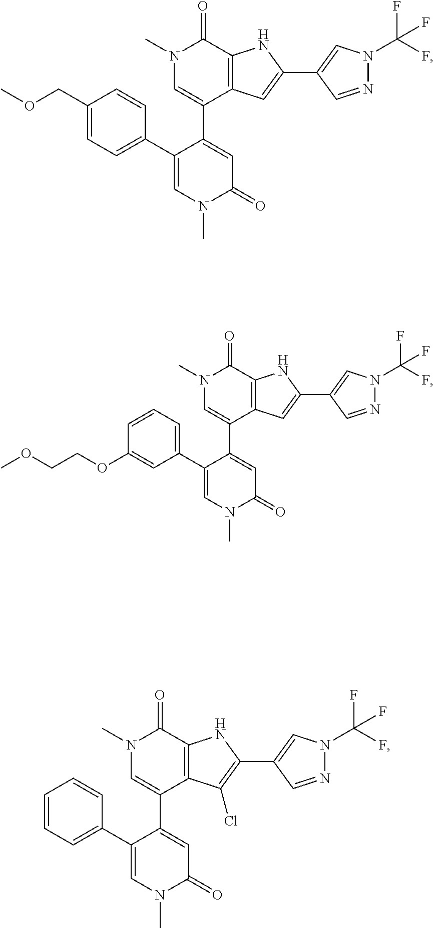 [SMILES: COCc1ccc(-c2cn(C)c(=O)cc2-c2cn(C)c(=O)c3[nH]c(-c4cnn(C(F)(F)F)c4)cc23)cc1 ; COCCOc1cccc(-c2cn(C)c(=O)cc2-c2cn(C)c(=O)c3[nH]c(-c4cnn(C(F)(F)F)c4)cc23)c1 ; Cn1cc(-c2ccccc2)c(-c2cn(C)c(=O)c3[nH]c(-c4cnn(C(F)(F)F)c4)c(Cl)c23)cc1=O]

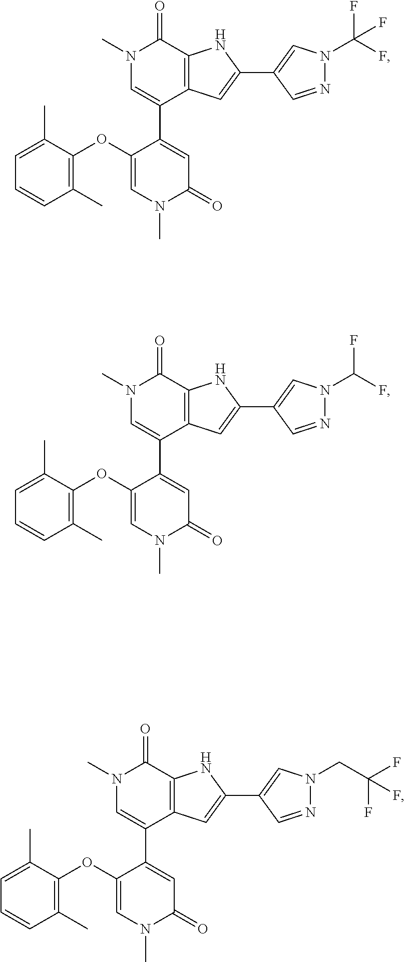 [SMILES: Cc1cccc(C)c1Oc1cn(C)c(=O)cc1-c1cn(C)c(=O)c2[nH]c(-c3cnn(C(F)(F)F)c3)cc12 ; Cc1cccc(C)c1Oc1cn(C)c(=O)cc1-c1cn(C)c(=O)c2[nH]c(-c3cnn(C(F)F)c3)cc12 ; Cc1cccc(C)c1Oc1cn(C)c(=O)cc1-c1cn(C)c(=O)c2[nH]c(-c3cnn(CC(F)(F)F)c3)cc12]

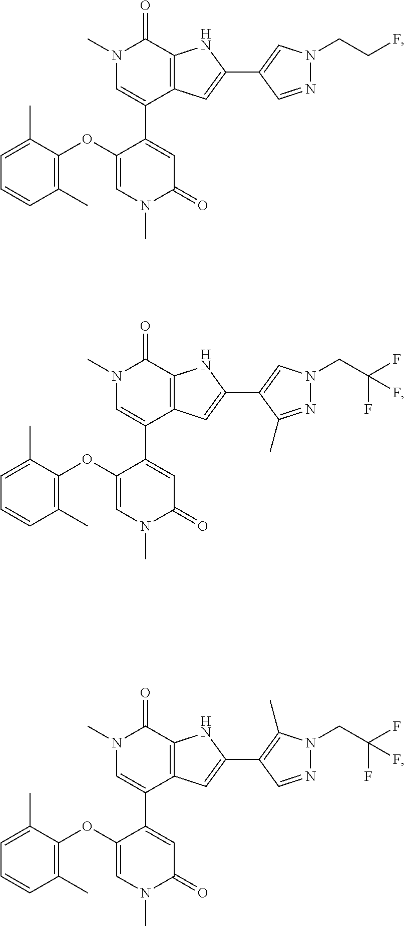 [SMILES: Cc1cccc(C)c1Oc1cn(C)c(=O)cc1-c1cn(C)c(=O)c2[nH]c(-c3cnn(CCF)c3)cc12 ; Cc1cccc(C)c1Oc1cn(C)c(=O)cc1-c1cn(C)c(=O)c2[nH]c(-c3cn(CC(F)(F)F)nc3C)cc12 ; Cc1cccc(C)c1Oc1cn(C)c(=O)cc1-c1cn(C)c(=O)c2[nH]c(-c3cnn(CC(F)(F)F)c3C)cc12]

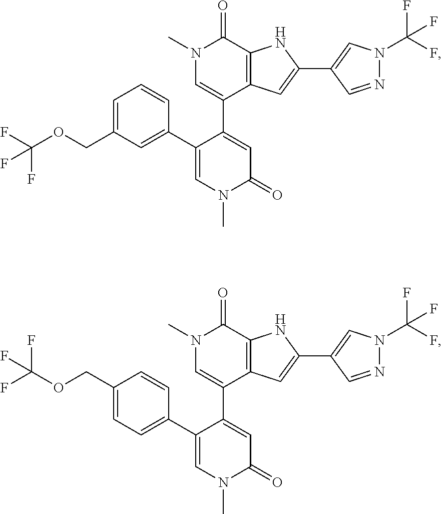 [SMILES: Cn1cc(-c2cccc(COC(F)(F)F)c2)c(-c2cn(C)c(=O)c3[nH]c(-c4cnn(C(F)(F)F)c4)cc23)cc1=O ; Cn1cc(-c2ccc(COC(F)(F)F)cc2)c(-c2cn(C)c(=O)c3[nH]c(-c4cnn(C(F)(F)F)c4)cc23)cc1=O]

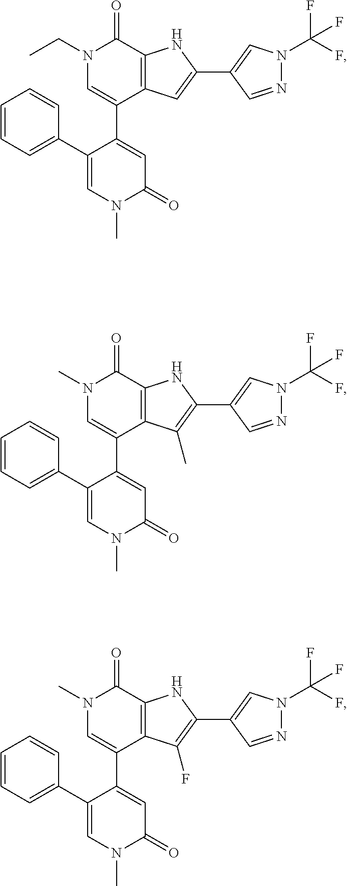 [SMILES: CCn1cc(-c2cc(=O)n(C)cc2-c2ccccc2)c2cc(-c3cnn(C(F)(F)F)c3)[nH]c2c1=O ; Cc1c(-c2cnn(C(F)(F)F)c2)[nH]c2c(=O)n(C)cc(-c3cc(=O)n(C)cc3-c3ccccc3)c12 ; Cn1cc(-c2ccccc2)c(-c2cn(C)c(=O)c3[nH]c(-c4cnn(C(F)(F)F)c4)c(F)c23)cc1=O]

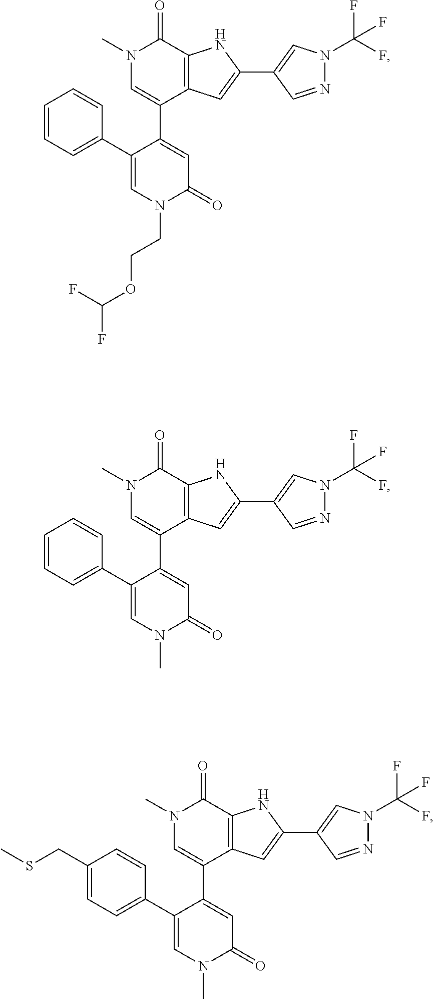 [SMILES: Cn1cc(-c2cc(=O)n(CCOC(F)F)cc2-c2ccccc2)c2cc(-c3cnn(C(F)(F)F)c3)[nH]c2c1=O ; Cn1cc(-c2ccccc2)c(-c2cn(C)c(=O)c3[nH]c(-c4cnn(C(F)(F)F)c4)cc23)cc1=O ; CSCc1ccc(-c2cn(C)c(=O)cc2-c2cn(C)c(=O)c3[nH]c(-c4cnn(C(F)(F)F)c4)cc23)cc1]

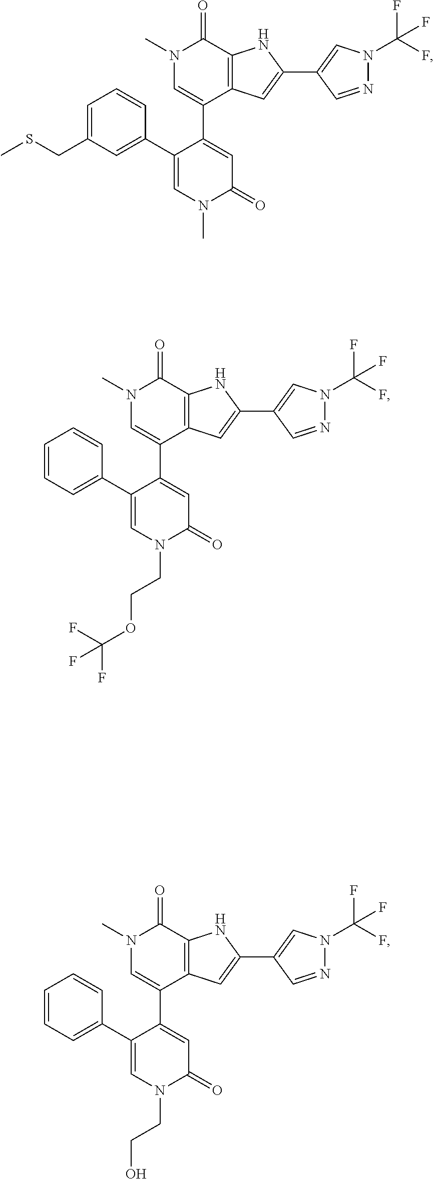 [SMILES: CSCc1cccc(-c2cn(C)c(=O)cc2-c2cn(C)c(=O)c3[nH]c(-c4cnn(C(F)(F)F)c4)cc23)c1 ; Cn1cc(-c2cc(=O)n(CCOC(F)(F)F)cc2-c2ccccc2)c2cc(-c3cnn(C(F)(F)F)c3)[nH]c2c1=O ; Cn1cc(-c2cc(=O)n(CCO)cc2-c2ccccc2)c2cc(-c3cnn(C(F)(F)F)c3)[nH]c2c1=O]

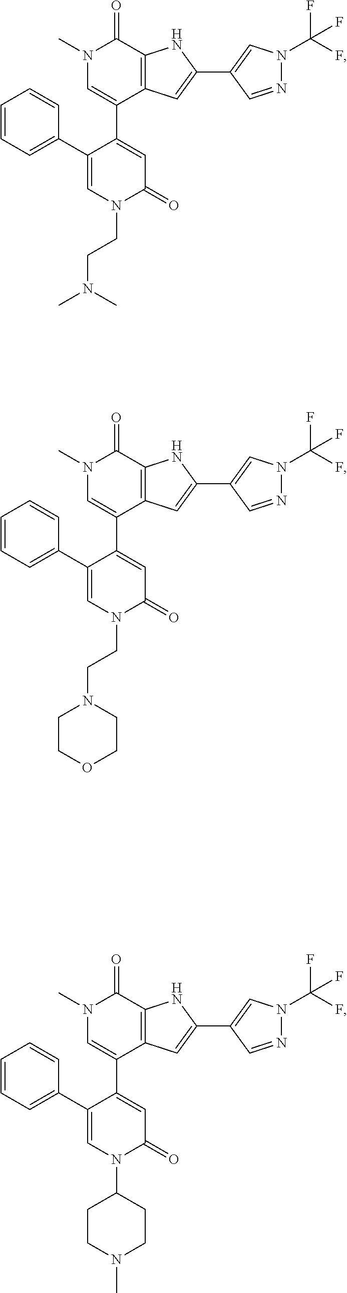 [SMILES: CN(C)CCn1cc(-c2ccccc2)c(-c2cn(C)c(=O)c3[nH]c(-c4cnn(C(F)(F)F)c4)cc23)cc1=O ; Cn1cc(-c2cc(=O)n(CCN3CCOCC3)cc2-c2ccccc2)c2cc(-c3cnn(C(F)(F)F)c3)[nH]c2c1=O ; CN1CCC(n2cc(-c3ccccc3)c(-c3cn(C)c(=O)c4[nH]c(-c5cnn(C(F)(F)F)c5)cc34)cc2=O)CC1]

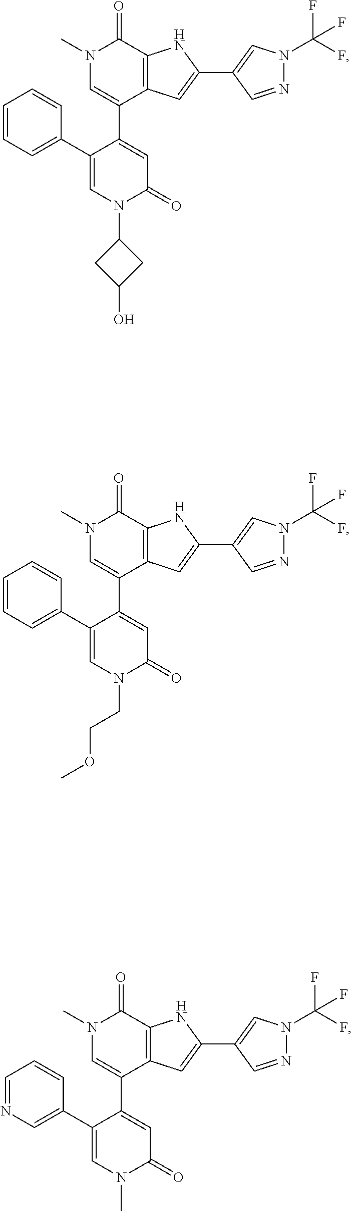 [SMILES: Cn1cc(-c2cc(=O)n(C3CC(O)C3)cc2-c2ccccc2)c2cc(-c3cnn(C(F)(F)F)c3)[nH]c2c1=O ; COCCn1cc(-c2ccccc2)c(-c2cn(C)c(=O)c3[nH]c(-c4cnn(C(F)(F)F)c4)cc23)cc1=O ; Cn1cc(-c2cccnc2)c(-c2cn(C)c(=O)c3[nH]c(-c4cnn(C(F)(F)F)c4)cc23)cc1=O]

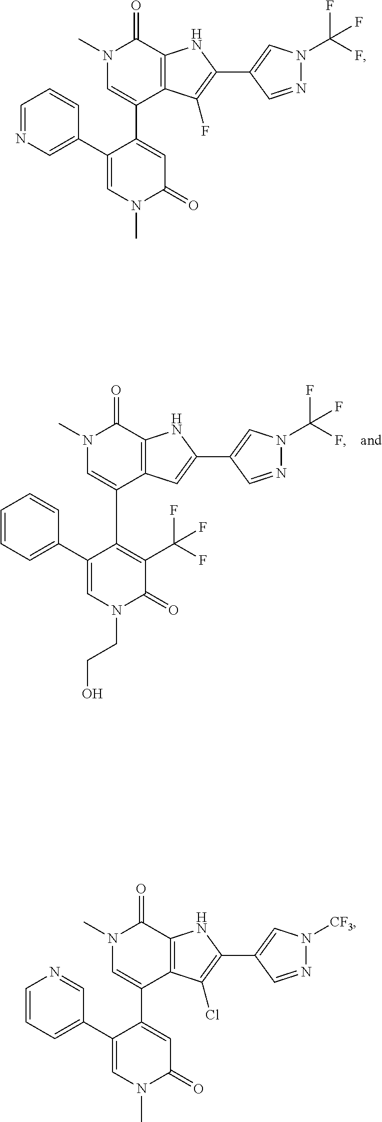 [SMILES: Cn1cc(-c2cccnc2)c(-c2cn(C)c(=O)c3[nH]c(-c4cnn(C(F)(F)F)c4)c(F)c23)cc1=O ; Cn1cc(-c2c(-c3ccccc3)cn(CCO)c(=O)c2C(F)(F)F)c2cc(-c3cnn(C(F)(F)F)c3)[nH]c2c1=O ; Cn1cc(-c2cccnc2)c(-c2cn(C)c(=O)c3[nH]c(-c4cnn(C(F)(F)F)c4)c(Cl)c23)cc1=O]

or a pharmaceutically acceptable salt or N-oxide thereof.

In some embodiments, is the present disclosure provides a compound selected from:

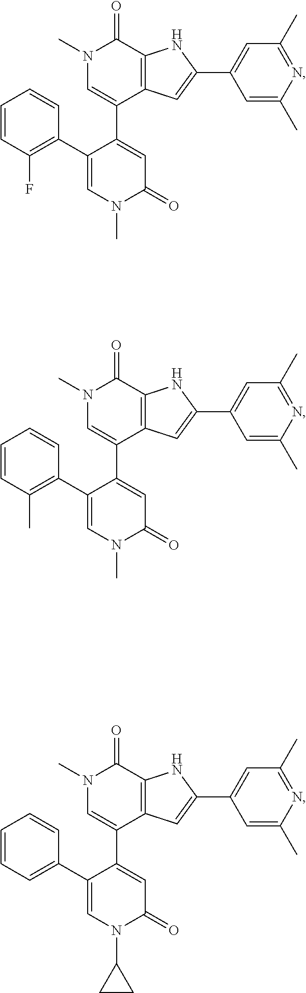 [SMILES: Cc1cc(-c2cc3c(-c4cc(=O)n(C)cc4-c4ccccc4F)cn(C)c(=O)c3[nH]2)cc(C)n1 ; Cc1cc(-c2cc3c(-c4cc(=O)n(C)cc4-c4ccccc4C)cn(C)c(=O)c3[nH]2)cc(C)n1 ; Cc1cc(-c2cc3c(-c4cc(=O)n(C5CC5)cc4-c4ccccc4)cn(C)c(=O)c3[nH]2)cc(C)n1]

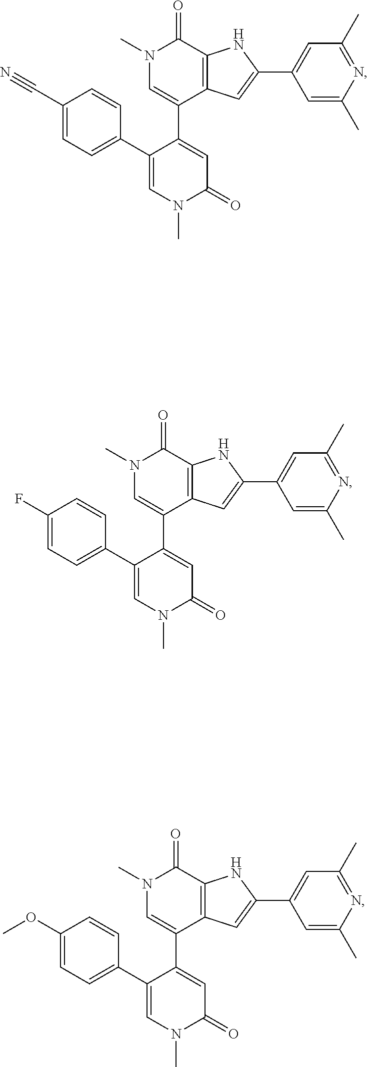

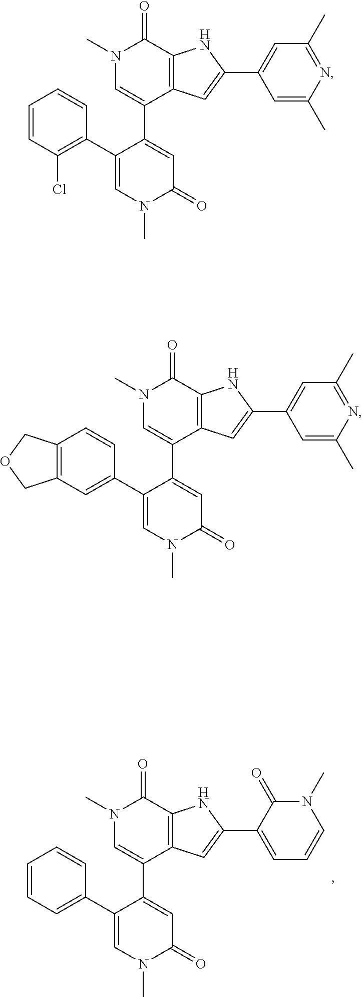 [SMILES: Cc1cc(-c2cc3c(-c4cc(=O)n(C)cc4-c4ccccc4Cl)cn(C)c(=O)c3[nH]2)cc(C)n1 ; Cc1cc(-c2cc3c(-c4cc(=O)n(C)cc4-c4ccc5c(c4)COC5)cn(C)c(=O)c3[nH]2)cc(C)n1 ; Cn1cc(-c2ccccc2)c(-c2cn(C)c(=O)c3[nH]c(-c4cccn(C)c4=O)cc23)cc1=O]

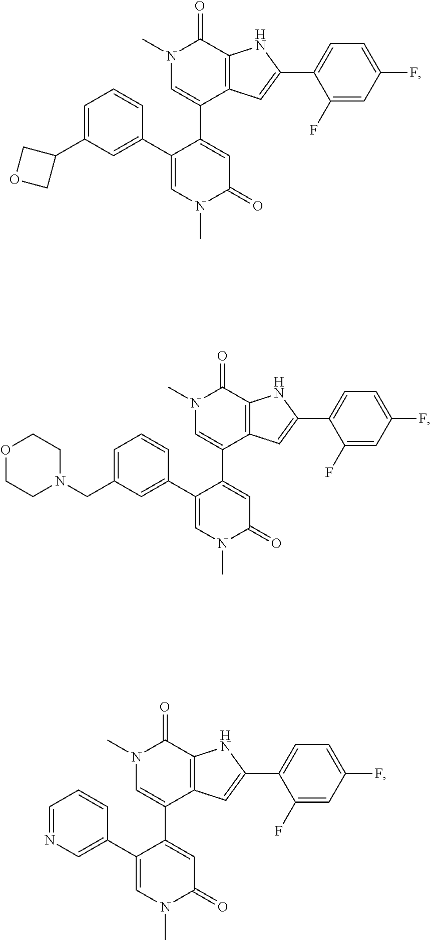 [SMILES: Cn1cc(-c2cccc(C3COC3)c2)c(-c2cn(C)c(=O)c3[nH]c(-c4ccc(F)cc4F)cc23)cc1=O ; Cn1cc(-c2cccc(CN3CCOCC3)c2)c(-c2cn(C)c(=O)c3[nH]c(-c4ccc(F)cc4F)cc23)cc1=O ; Cn1cc(-c2cccnc2)c(-c2cn(C)c(=O)c3[nH]c(-c4ccc(F)cc4F)cc23)cc1=O]

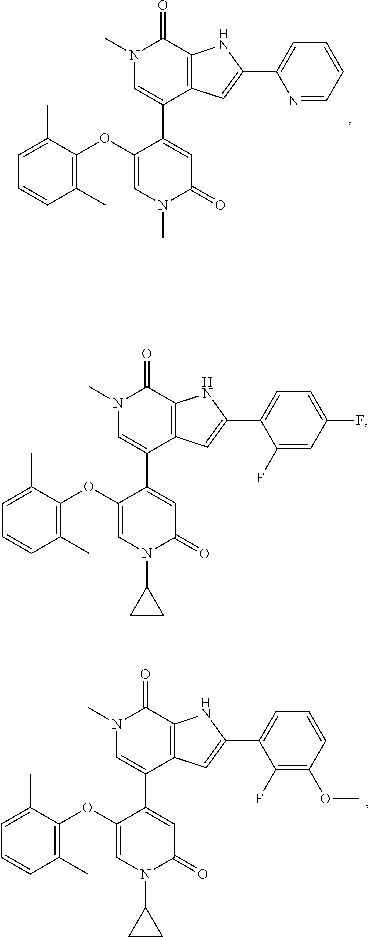 [SMILES: Cc1cccc(C)c1Oc1cn(C)c(=O)cc1-c1cn(C)c(=O)c2[nH]c(-c3ccccn3)cc12 ; Cc1cccc(C)c1Oc1cn(C2CC2)c(=O)cc1-c1cn(C)c(=O)c2[nH]c(-c3ccc(F)cc3F)cc12 ; COc1cccc(-c2cc3c(-c4cc(=O)n(C5CC5)cc4Oc4c(C)cccc4C)cn(C)c(=O)c3[nH]2)c1F]

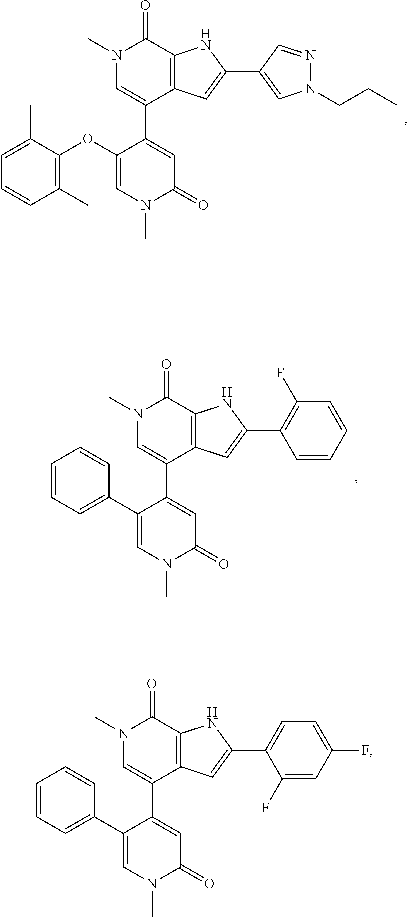 [SMILES: CCCn1cc(-c2cc3c(-c4cc(=O)n(C)cc4Oc4c(C)cccc4C)cn(C)c(=O)c3[nH]2)cn1 ; Cn1cc(-c2ccccc2)c(-c2cn(C)c(=O)c3[nH]c(-c4ccccc4F)cc23)cc1=O ; Cn1cc(-c2ccccc2)c(-c2cn(C)c(=O)c3[nH]c(-c4ccc(F)cc4F)cc23)cc1=O]

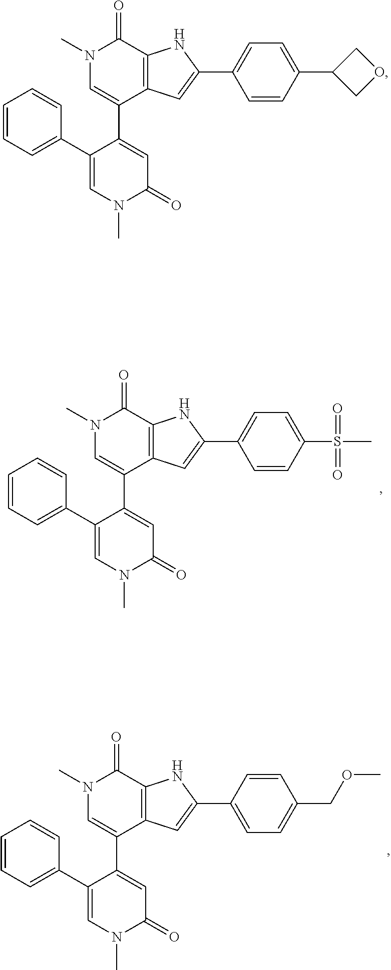 [SMILES: Cn1cc(-c2ccccc2)c(-c2cn(C)c(=O)c3[nH]c(-c4ccc(C5COC5)cc4)cc23)cc1=O ; Cn1cc(-c2ccccc2)c(-c2cn(C)c(=O)c3[nH]c(-c4ccc(S(C)(=O)=O)cc4)cc23)cc1=O ; COCc1ccc(-c2cc3c(-c4cc(=O)n(C)cc4-c4ccccc4)cn(C)c(=O)c3[nH]2)cc1]

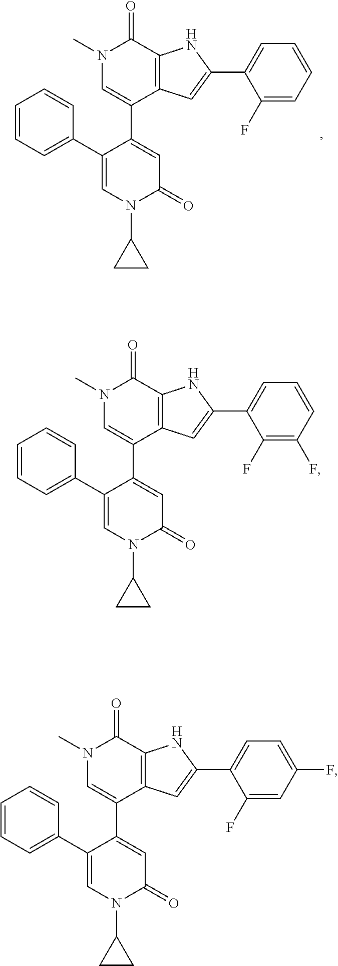 [SMILES: Cn1cc(-c2cc(=O)n(C3CC3)cc2-c2ccccc2)c2cc(-c3ccccc3F)[nH]c2c1=O ; Cn1cc(-c2cc(=O)n(C3CC3)cc2-c2ccccc2)c2cc(-c3cccc(F)c3F)[nH]c2c1=O ; Cn1cc(-c2cc(=O)n(C3CC3)cc2-c2ccccc2)c2cc(-c3ccc(F)cc3F)[nH]c2c1=O]

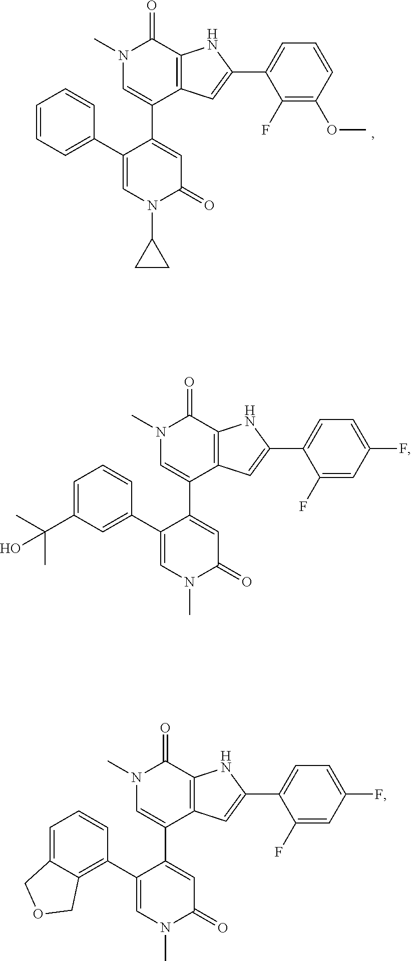

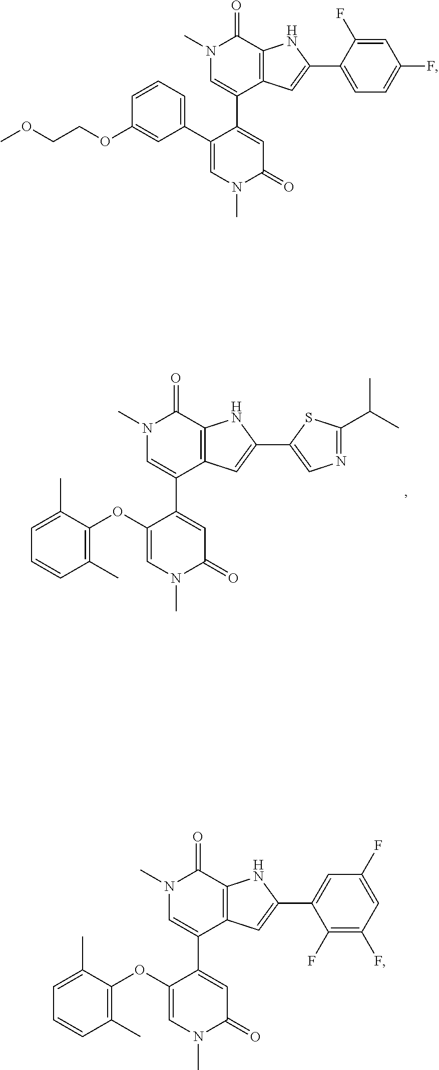 [SMILES: COCCOc1cccc(-c2cn(C)c(=O)cc2-c2cn(C)c(=O)c3[nH]c(-c4ccc(F)cc4F)cc23)c1 ; Cc1cccc(C)c1Oc1cn(C)c(=O)cc1-c1cn(C)c(=O)c2[nH]c(-c3cnc(C(C)C)s3)cc12 ; Cc1cccc(C)c1Oc1cn(C)c(=O)cc1-c1cn(C)c(=O)c2[nH]c(-c3cc(F)cc(F)c3F)cc12]

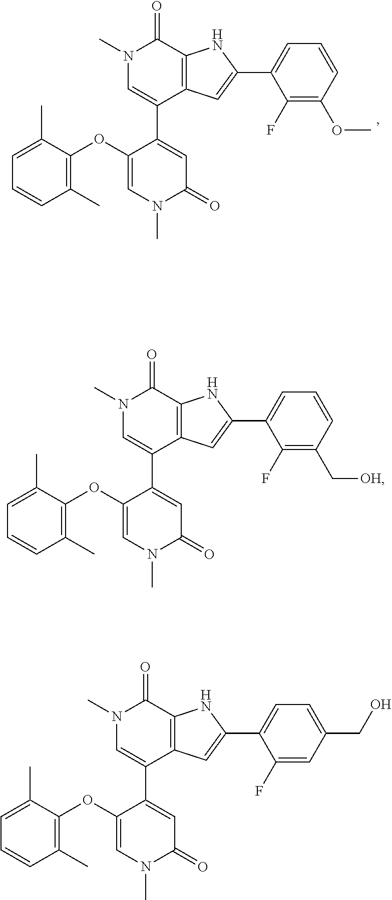 [SMILES: COc1cccc(-c2cc3c(-c4cc(=O)n(C)cc4Oc4c(C)cccc4C)cn(C)c(=O)c3[nH]2)c1F ; Cc1cccc(C)c1Oc1cn(C)c(=O)cc1-c1cn(C)c(=O)c2[nH]c(-c3cccc(CO)c3F)cc12 ; Cc1cccc(C)c1Oc1cn(C)c(=O)cc1-c1cn(C)c(=O)c2[nH]c(-c3ccc(CO)cc3F)cc12]

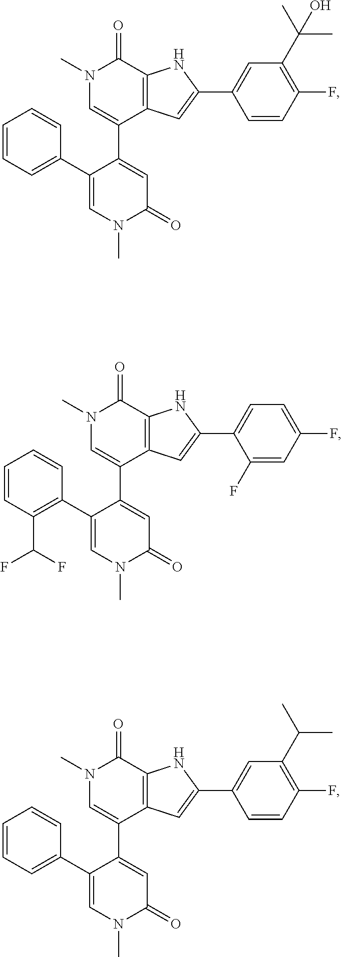 [SMILES: Cn1cc(-c2ccccc2)c(-c2cn(C)c(=O)c3[nH]c(-c4ccc(F)c(C(C)(C)O)c4)cc23)cc1=O ; Cn1cc(-c2ccccc2C(F)F)c(-c2cn(C)c(=O)c3[nH]c(-c4ccc(F)cc4F)cc23)cc1=O ; CC(C)c1cc(-c2cc3c(-c4cc(=O)n(C)cc4-c4ccccc4)cn(C)c(=O)c3[nH]2)ccc1F]

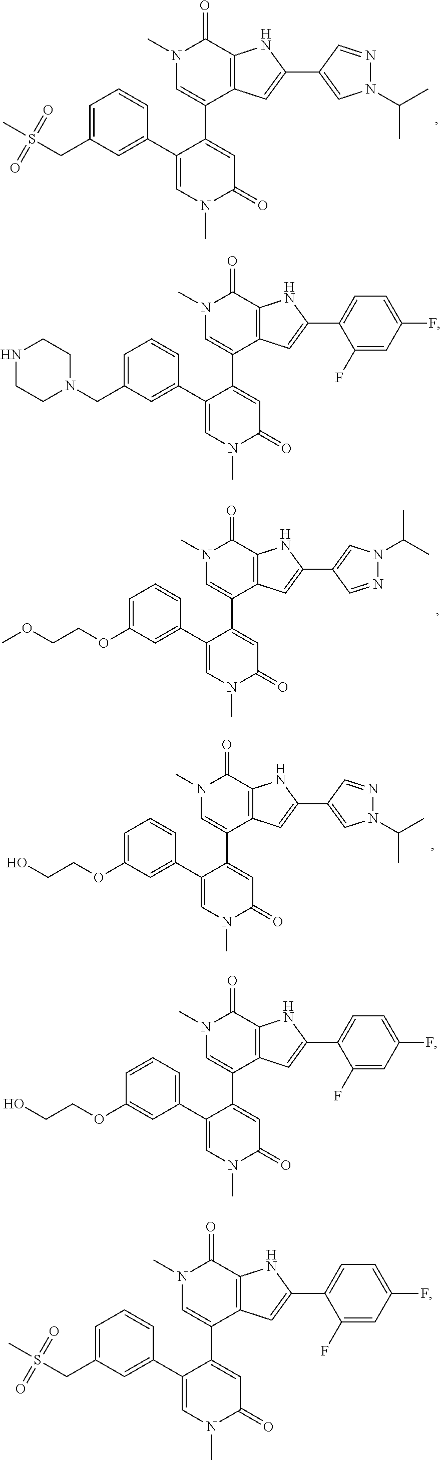

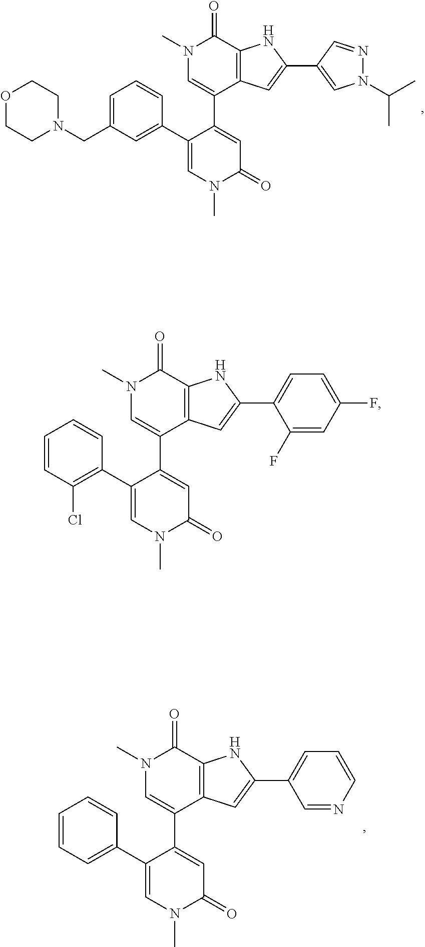 [SMILES: CC(C)n1cc(-c2cc3c(-c4cc(=O)n(C)cc4-c4cccc(CN5CCOCC5)c4)cn(C)c(=O)c3[nH]2)cn1 ; Cn1cc(-c2ccccc2Cl)c(-c2cn(C)c(=O)c3[nH]c(-c4ccc(F)cc4F)cc23)cc1=O ; Cn1cc(-c2ccccc2)c(-c2cn(C)c(=O)c3[nH]c(-c4cccnc4)cc23)cc1=O]

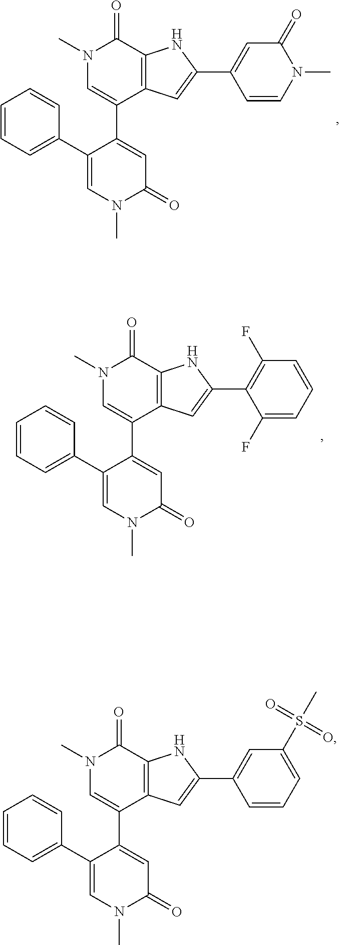 [SMILES: Cn1ccc(-c2cc3c(-c4cc(=O)n(C)cc4-c4ccccc4)cn(C)c(=O)c3[nH]2)cc1=O ; Cn1cc(-c2ccccc2)c(-c2cn(C)c(=O)c3[nH]c(-c4c(F)cccc4F)cc23)cc1=O ; Cn1cc(-c2ccccc2)c(-c2cn(C)c(=O)c3[nH]c(-c4cccc(S(C)(=O)=O)c4)cc23)cc1=O]

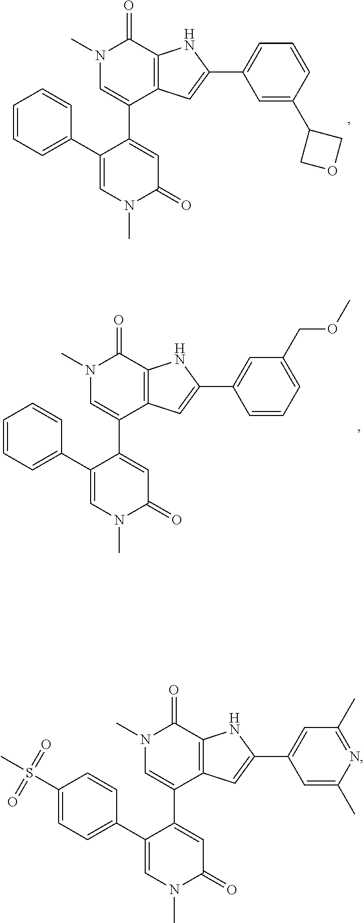

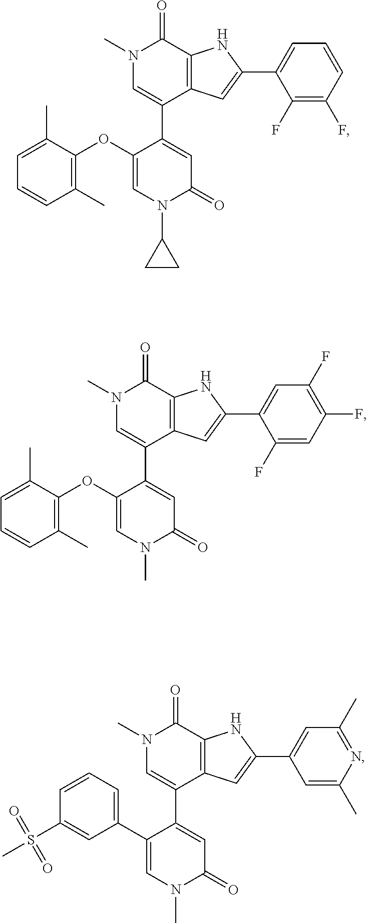

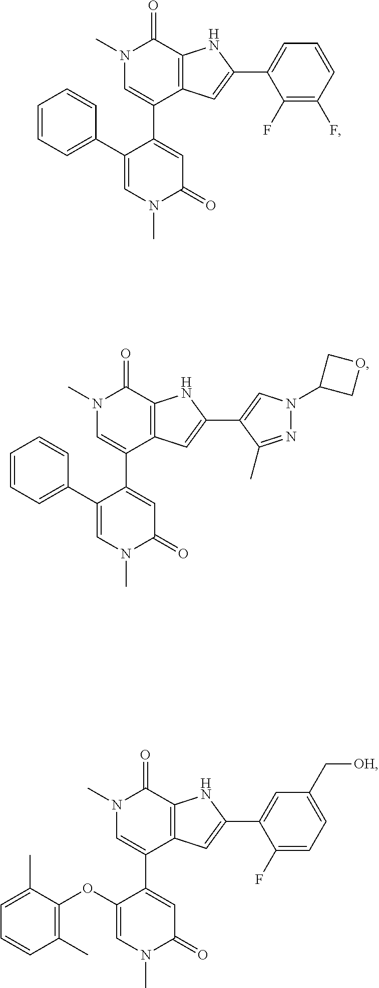 [SMILES: Cn1cc(-c2ccccc2)c(-c2cn(C)c(=O)c3[nH]c(-c4cccc(F)c4F)cc23)cc1=O ; Cc1nn(C2COC2)cc1-c1cc2c(-c3cc(=O)n(C)cc3-c3ccccc3)cn(C)c(=O)c2[nH]1 ; Cc1cccc(C)c1Oc1cn(C)c(=O)cc1-c1cn(C)c(=O)c2[nH]c(-c3cc(CO)ccc3F)cc12]

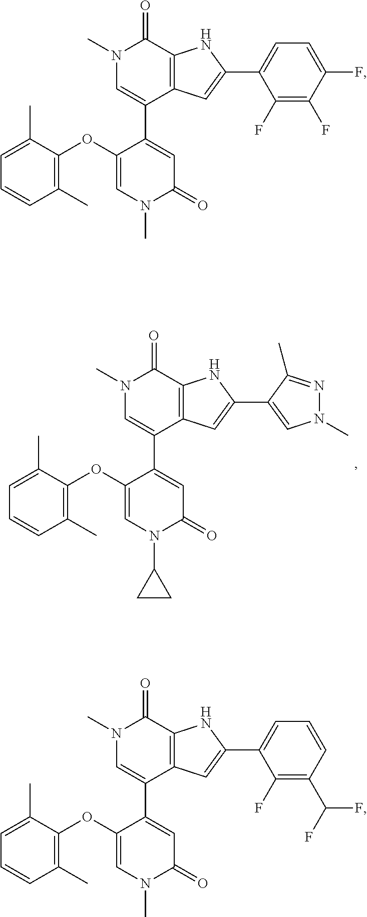 [SMILES: Cc1cccc(C)c1Oc1cn(C)c(=O)cc1-c1cn(C)c(=O)c2[nH]c(-c3ccc(F)c(F)c3F)cc12 ; Cc1cccc(C)c1Oc1cn(C2CC2)c(=O)cc1-c1cn(C)c(=O)c2[nH]c(-c3cn(C)nc3C)cc12 ; Cc1cccc(C)c1Oc1cn(C)c(=O)cc1-c1cn(C)c(=O)c2[nH]c(-c3cccc(C(F)F)c3F)cc12]

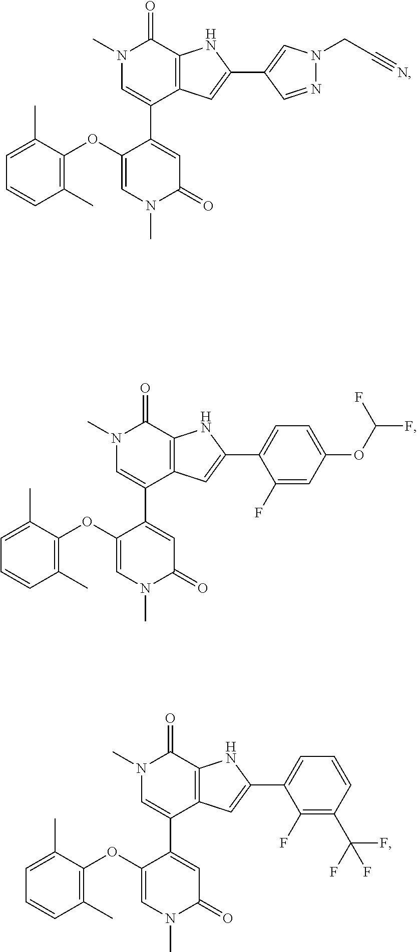 [SMILES: Cc1cccc(C)c1Oc1cn(C)c(=O)cc1-c1cn(C)c(=O)c2[nH]c(-c3cnn(CC#N)c3)cc12 ; Cc1cccc(C)c1Oc1cn(C)c(=O)cc1-c1cn(C)c(=O)c2[nH]c(-c3ccc(OC(F)F)cc3F)cc12 ; Cc1cccc(C)c1Oc1cn(C)c(=O)cc1-c1cn(C)c(=O)c2[nH]c(-c3cccc(C(F)(F)F)c3F)cc12]

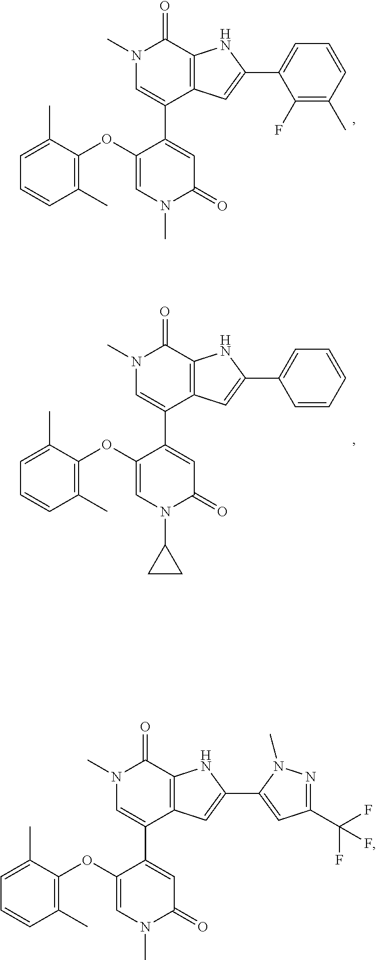 [SMILES: Cc1cccc(-c2cc3c(-c4cc(=O)n(C)cc4Oc4c(C)cccc4C)cn(C)c(=O)c3[nH]2)c1F ; Cc1cccc(C)c1Oc1cn(C2CC2)c(=O)cc1-c1cn(C)c(=O)c2[nH]c(-c3ccccc3)cc12 ; Cc1cccc(C)c1Oc1cn(C)c(=O)cc1-c1cn(C)c(=O)c2[nH]c(-c3cc(C(F)(F)F)nn3C)cc12]

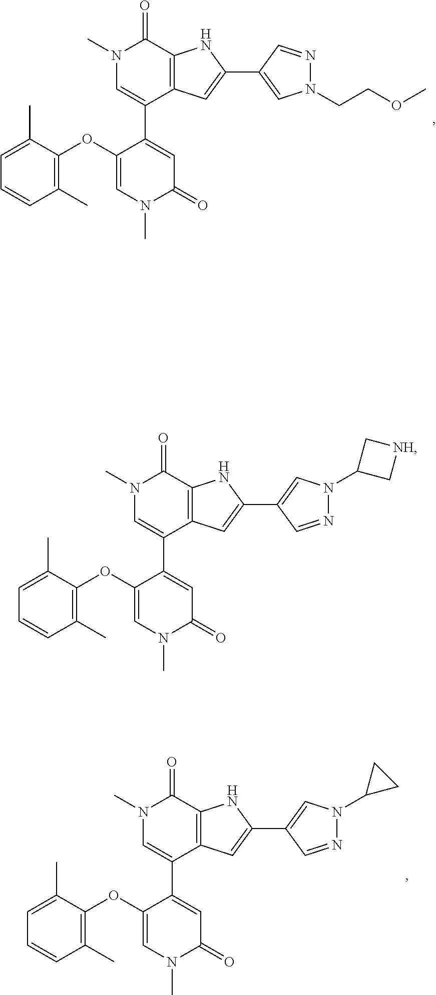 [SMILES: COCCn1cc(-c2cc3c(-c4cc(=O)n(C)cc4Oc4c(C)cccc4C)cn(C)c(=O)c3[nH]2)cn1 ; Cc1cccc(C)c1Oc1cn(C)c(=O)cc1-c1cn(C)c(=O)c2[nH]c(-c3cnn(C4CNC4)c3)cc12 ; Cc1cccc(C)c1Oc1cn(C)c(=O)cc1-c1cn(C)c(=O)c2[nH]c(-c3cnn(C4CC4)c3)cc12]

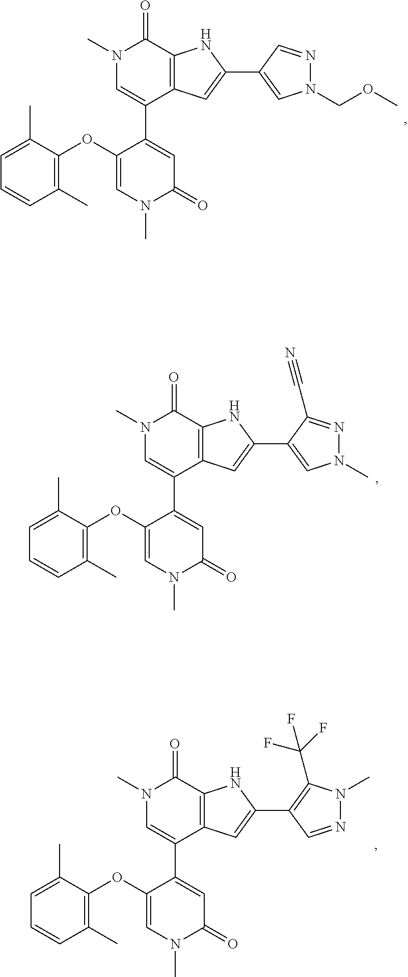 [SMILES: COCn1cc(-c2cc3c(-c4cc(=O)n(C)cc4Oc4c(C)cccc4C)cn(C)c(=O)c3[nH]2)cn1 ; Cc1cccc(C)c1Oc1cn(C)c(=O)cc1-c1cn(C)c(=O)c2[nH]c(-c3cn(C)nc3C#N)cc12 ; Cc1cccc(C)c1Oc1cn(C)c(=O)cc1-c1cn(C)c(=O)c2[nH]c(-c3cnn(C)c3C(F)(F)F)cc12]

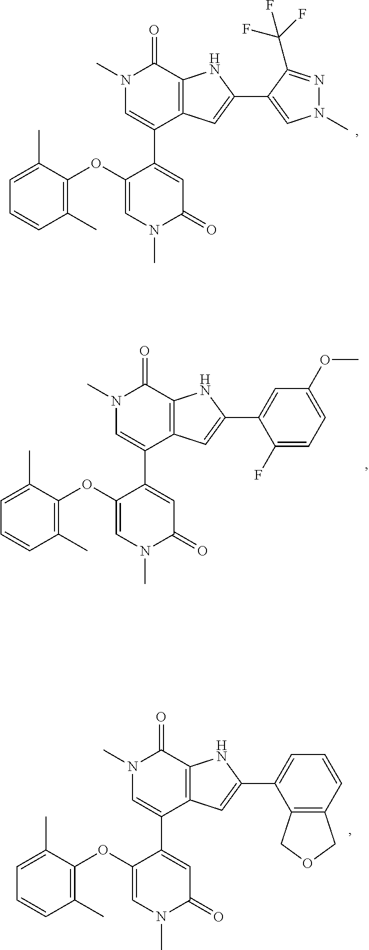 [SMILES: Cc1cccc(C)c1Oc1cn(C)c(=O)cc1-c1cn(C)c(=O)c2[nH]c(-c3cn(C)nc3C(F)(F)F)cc12 ; COc1ccc(F)c(-c2cc3c(-c4cc(=O)n(C)cc4Oc4c(C)cccc4C)cn(C)c(=O)c3[nH]2)c1 ; Cc1cccc(C)c1Oc1cn(C)c(=O)cc1-c1cn(C)c(=O)c2[nH]c(-c3cccc4c3COC4)cc12]

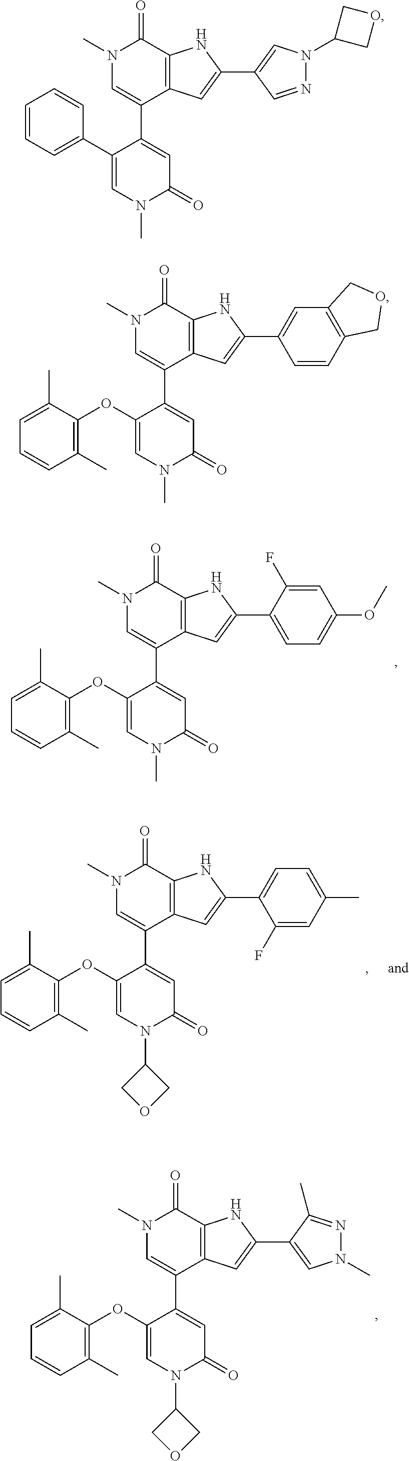

or a pharmaceutically acceptable salt or N-oxide thereof.

In some embodiments, the present disclosure provides a pharmaceutical composition comprising a compound disclosed herein, or a pharmaceutically acceptable salt thereof, or N-oxide thereof, and one or more pharmaceutically acceptable excipients.

In some embodiments, the present disclosure provides a method of treating a disease or disorder selected from one or more of an inflammatory disease or disorder, an immune disease or disorder, and an autoimmune disease or disorder, comprising administering to a warm-blooded animal a therapeutically effective amount of a compound disclosed herein, or a pharmaceutically acceptable salt thereof, or a N-oxide thereof.

In some embodiments, the disease or disorder is a joint disease or disorder or a joint-related disease or disorder.

In some embodiments, the disease or disorder is selected from arthritis, bursitis, Ehlers-Danlos syndrome, epicondylitis, Felty Syndrome, gouty arthritis, psoriatic arthritis, osteoarthritis, rheumatoid arthritis, Still's disease, tenosynovitis, synovitis, Sjögren's Syndrome, Lyme disease, Whipple disease, bone cancer, lupus, and other autoimmune joint disorders.

In some embodiments, the joint disease or disorder or the joint-related disease or disorder is an arthritis. In some embodiments, the arthritis is rheumatoid arthritis. In some embodiments, the disease or disorder is a fibrotic disease or disorder. In some embodiments, the disease or disorder is renal fibrosis. In some embodiments, the disease or disorder is pulmonary fibrosis. In some embodiments, the disease or disorder is a skin disease or disorder. In some embodiments, the disease or disorder is psoriasis. In some embodiments, the disease or disorder is a lupus disease or disorder. In some embodiments, the disease or disorder is a MS or MS related disease or disorder. In some embodiments, the method of treatment disclosed herein reduces the severity of the disease or disorder.

### BRIEF DESCRIPTION OF THE DRAWINGS

FIGS. 1A-1B illustrate the plasma concentration of Compound A formulated in 1% methylcellulose (FIG. 1A) and propylene glycol (FIG. 1B) in rats after oral administration (per os, PO). FIG. 1C provides a comparison of mean plasma concentrations for the two formulations.

FIGS. 2A-2B illustrate the change in plasma concentration of Compound A over time when prepared in a DMSO-based formulation. FIG. 2A depicts individual results for animals, and FIG. 2B shows the mean plasm concentration after oral delivery (PO).

FIGS. 3A-3C depict changes in plasma concentration for ABBV-744 when administered intravenously (IV; FIG. 3A) or orally (PO; FIG. 3B). FIG. 3C provides a comparison of the results for the two different routes of administration. FIG. 3D illustrates the mean pharmacokinetics for ABBV-744 in relation to the free EC50 BD1 and the free EC50 BD2 BRD4.

FIGS. 4A-4D depict the pharmacokinetics of Compound A when delivered at two different doses in a propylene glycol-based formulation. FIG. 4A shows plasma concentration of Compound A when administered at a dose of 1 mg/kg. FIG. 4B shows plasma concentration of Compound A when administered at a dose of 0.2 mg/kg. FIG. 4C provides a comparison of the mean plasma concentration for the two doses (1 mg/kg=square, 0.2 mg/kg=diamond), and FIG. 4D shows the plasma concentrations of the various doses of Compound A in relation to the free EC50 for BD1 and the free EC50 for BD2 (10 mg/kg=line, 1 mg/kg=diamond, 0.2 mg/kg=circle).

FIGS. 5A-5C depict the pharmacokinetics of Compound A in a propylene glycol-based formulation when delivered via two different routes of administration: intravenous (IV; FIG. 5A) and oral (PO; FIG. 5B) in beagle dogs. FIG. 5C provides a comparison of mean Compound A plasma concentration for the two routes of administration.

FIGS. 6A-6B illustrate the pharmacokinetics of Compound A in a HPPCD formulation. FIG. 6A shows results for individual animals, and FIG. 6B shows the mean change in plasma concentration (IV).

FIGS. 7A-7L show plasma concentration of Compound A in beagle dogs after administration at a range of doses (5 mg/kg, 10 mg/kg, and 20 mg/kg) on day 1 (FIGS. 7A-7C, respectively) and on day 5 (FIGS. 7D-7F, respectively). FIG. 7G provides a comparison of the mean plasma concentration for the data presented in FIGS. 7A-7F. FIG. 7H shows body weight change after administration of the 5 mg/kg dose, FIG. 7I shows body weight change after administration of the 10 mg/kg dose, FIG. 7J shows body weight change after administration of the 20 mg/kg dose, and FIG. 7K shows a comparison of body weight changes for the three doses. FIG. 7L shows the plasma concentration of Compound A at three different doses (1 mg/kg, 5 mg/kg, and 10 mg/kg) relative to the free EC50 for BD1 and the free EC50 for BD2, which had been previously determined. FIG. 7M shows the change in plasma concentration of Compound A over a 24-hour period following 14 days of daily administration of Compound A at three doses (1 mg/kg, 3 mg/kg, and 10 mg/kg). FIG. 7N shows the absence of impact on platelet count of administration of Compound A at three doses (1 mg/kg, 3 mg/kg, and 10 mg/kg) after daily administration for 14 days.

FIGS. 8A-8C show inhibition of CXCL10 (FIG. 7A), IL-17A (FIG. 7B), and IL-22 (FIG. 7C) by Compound A.

FIGS. 9A-9G illustrate results from the study of Compound A in the imiquimod-induced psoriasis model. FIG. 9A depicts the percent change in psoriasis area and severity index (PASI) scores for animals. FIG. 9B depicts the mean percent change in PASI scores at day 15 relative to baseline scores. FIG. 9C depicts overall changes in PASI scores throughout the study, and FIG. 9D depicts mean PASI scores at day 15. FIG. 9E depicts mean changes in erythema scoring on the skin where psoriasis is induced. FIG. 9F depicts mean changes in induration scoring on the skin where psoriasis is induced. FIG. 9G depicts mean changes in peeling scoring on the skin where psoriasis is induced.

FIGS. 10A and 10B depict changes in body weight (FIG. 10A) and mean body weight changes over days 1-8 and 8-15 (FIG. 10B).

FIG. 11 illustrates the mean spleen:weight ratio for animals at day 15.

FIG. 12A shows mean scratching score, FIG. 12B shows mean licking score, and FIG. 12C shows the combined mean scratching and licking score for animals. FIGS. 12D and 12E show the use of enrichment over the course of the study (FIG. 12D) and mean scores for use of enrichment over days 1-8 and days 8-15 (FIG. 12E). FIG. 12F illustrates the mean number of rearings in animals, and FIG. 12G shows the mean distance travelled by animals in their cages.

FIGS. 13A-13K show representative images of the use of enrichment by animals in the respective groups: FIG. 13A (sham), FIG. 13B (IMI+Vehicle), FIG. 13C (IMI+Compound A 1 mg/kg), FIG. 13D (IMI+Compound A 3 mg/kg), FIG. 13E (IMI+Compound A 10 mg/kg). FIG. 13F (Compound B 1 mg/kg), FIG. 13G (Compound B 3 mg/kg), FIG. 13H (Compound B 10 mg/kg), FIG. 13I (IMI+Apremilast 3 mg/kg), FIG. 13J (IMI+Deucravacitinib 3 mg/kg), FIG. 13K (IMI+Clobetasol 0.05%).

FIGS. 14A-14H provide representative images of dorsal depilated mice in the placebo group at day 8 (FIG. 14A) and day 15 (FIG. 14E), the Compound A (3 mg/kg) treatment group at day 8 (FIG. 14B) and day 15 (FIG. 14F), the deucravacitinib (3 mg/kg) treatment group at day 8 (FIG. 14C) and day 15 (FIG. 14G), and the Compound B (10 mg/kg) treatment at day 8 (FIG. 14D) and day 15 (FIG. 14H).

FIGS. 15A-15B depict levels of cytokines measured in animals where psoriasis has been induced and receiving various treatments (vehicle, Compound A (1 mg/kg), Compound A (3 mg/kg), Compound A (10 mg/kg), and deucravacitinib (3 mg/kg). FIG. 15A shows levels of IL-17 and IL-22. FIG. 15B shows levels of IL-1β, IL-6, TNF-α, and IL-23.

FIG. 16 provides a depiction of the CIA study protocol.

FIG. 17A depicts actual body weight in grams of Lewis rats over 21-day following induction of collagen-induced arthritis under various treatment conditions. FIG. 17B depicts calculated percentage change in body weight for animals over the study course.

FIG. 18A depicts paw volume of left hind paw of Lewis rats over 21-day following induction of collagen-induced arthritis under various treatment conditions. FIG. 18B depicts paw volume of right hind paw of Lewis rats over 21-day following induction of collagen-induced arthritis under various treatment conditions. FIG. 18C depicts mean volume of left and right hind paw for animals over the study course.

FIGS. 19A-19B show clinical scoring of arthritis symptoms in Lewis rats over 21-day following induction of collagen-induced arthritis under various treatment conditions.

FIGS. 20A-20B shows mean levels of rat anti-collagen IgG1 antibodies in animals at the end of the 21-day study. FIG. 20A also illustrates individual levels.

FIGS. 21A-21C depict the pharmacokinetic profile of Compound A in rats on day 0 (FIG. 21A) and day 21 (FIG. 21B) after oral administration in the CIA study. FIG. 21C shows a comparison of the mean plasma concentration for day 0 and day 21 results.

FIG. 22A depicts mean histopathological scores for tissue samples from animals in Groups 1-7. FIG. 22B shows histology scores for animals on day 21.

FIGS. 23A-23G depict representative tissue samples from animals in Groups 1-6 analyzed in the histopathological analysis. FIG. 23A shows a representative sample from Group 1 (No CIA). FIG. 23B shows a representative sample from Group 2 (CIA+Vehicle). FIG. 23C shows a representative sample from Group 3 (CIA+Dexamethasone) depicting tissue with no histopathological observations noted. FIG. 23D shows a representative sample from Group 4 (CIA+GSK620). FIG. 23E shows a representative sample from Group 5 (CIA+Compound A, 1 mg/kg). FIG. 23F shows a representative sample from Group 6 (CIA+Compound A, 3 mg/kg). FIG. 23G shows a representative sample from Group 7 (CIA+Compound A, 10 mg/kg). FIGS. 23H and 231 provide a comparison of tissue from vehicle treated animals in relation to animals receiving Compound A.

FIG. 24 shows the study design for the UUO rat renal fibrosis model.

FIG. 25 depicts mean body weight change (10A) of sham rats (circle), rats with UUO treated with vehicle (square), and UUO rats treated with Compound A (triangle).

FIG. 26A shows mean clinical histopathology score (including interstitial nephritis, collagen fiber deposition, and nephropathology). FIG. 26B-26C depict representative staining samples of tissue from rats with UUO treated with vehicle (26B) and from rats with UUO treated with Compound A (10 mg/kg) (26C).

FIG. 26D illustrates mean clinical histology scores in animals at study completion.

FIG. 27A depicts mean serum urea levels of sham rats (black bar), rats with UUO treated with vehicle (grey bar), and rats with UUO treated with Compound A (10 mg/kg) (white bar). FIGS. 27B-27C depict hydroxyproline levels in tissue of sham rats, rats with UUO treated with vehicle, and rats with UUO treated with Compound A (10 mg/kg). In FIG. 27B, mean hydroxyproline levels are depicted for sham rats (black bar), rats with UUO treated with vehicle (grey bar), and rats with UUO treated with Compound A (10 mg/kg) (white bar). FIG. 27C depicts individual results for sham rats (square), rats with UUO treated with vehicle (circle), and rats with UUO treated with Compound A (10 mg/kg) (triangle).

FIGS. 28A-28B show mean mRNA levels of tissue biomarkers (Col1a1, TGF-b1, MCP-1, IL-1b, IL-6, IL-17, TNF-α, and Timp1) in sham rats (black bar), rats with UUO treated with vehicle (grey), and rats with UUO treated with Compound A (10 mg/kg) (white bar). FIG. 28B overlays results for individual animals. FIG. 28C illustrates the change in expression of Col1a1, TGF-b1, IL-1b, IL-17, MC^{P}-1, and IL-6 in animals receiving treatment with Compound A relative to vehicle treated animals.

FIG. 29A shows mean percentage weight loss for animals in the IPF study over the 21-day study period.

FIG. 29B depicts mean percentage change in oxygen saturation levels for animals in the IPF study, taken every other day from Day 7 until Day 21.

FIG. 29C shows mean Ashcroft scoring for lung tissue recorded at Day 21 in the IPF study.

FIG. 29D shows mean hydroxyproline levels in lung lysates on Day 21 in the IPF study.

FIGS. 29E-29J show representative tissue samples stained with Masson's Trichrome. Hashed areas depict fibrosis and asterisks (*) depict normal respiratory zones. Conducting areas (“A”), sporadic airways (“B”), less severe localized fibrosis in airways (“C”), and less severe localized fibrosis in the parenchyma (“D”) are also marked in images.

FIG. 29K shows mean functional lung volume in animals in the IPF study. FIGS. 29L-29N also show representative CT images of lungs from animals in the control group where IPF was not induced (FIG. 29L), an IPF control group with saline treatment (FIG. 29M), and animals treated with Compound A 10 mg/kg group (FIG. 29N).

FIG. 30A depicts mean body weight gain from Week 11 to Week 19 of the lupus study, and FIG. 30B depicts the mean relative end body weight.

FIG. 30C shows proteinuria scores for animals in the lupus study from Week 10 until study completion at Week 19, and FIG. 30D shows the mean end proteinuria scores.

FIGS. 30E-30G show histopathological scores for total glomerular lesions (FIG. 30E), total tubular and interstitial lesions (FIG. 30F), and total kidney lesions (FIG. 30G) after study completion at Week 19. FIGS. 30H-30L show representative histology images prepared following study completion (Vehicle, FIG. 30H; Compound A 1 mg/kg, FIG. 30I; Compound A 3 mg/kg, FIG. 30J; Compound A 10 mg/kg, FIG. 30K; and Cyclophosphamide, FIG. 30L).

FIGS. 30M-30N show mean kidney weight (FIG. 30M) and mean spleen weight (FIG. 30N) after study completion at Week 19.

FIG. 31 depicts blood urea nitrogen (BUN) concentration (mg/dL) in serum after study completion at Week 19.

FIG. 32 shows levels of anti-dsDNA antibodies in serum at Weeks 11, 16 and 19.

FIG. 33 shows gastrointestinal villi from healthy animals treated with a pan-BD BET inhibitor (ABBV-075), vehicle, and BETi1 (10 mg/kg).

FIG. 34A shows the mean EAE score for animals over the course of the EAE study. FIG. 34B shows the mean maximum EAE score, FIG. 34C shows the mean day of onset of EAE, FIG. 34D shows the percent incidence of EAE, FIG. 34E shows the mean end EAE score, FIG. 34F shows the change in relative body weight over the course of the study, and FIG. 34G shows the mean relative end body weight for the different groups in the EAE study.

FIGS. 35A-35B show the mean concentration of IFNγ (FIG. 35A) and IL-12/IL-23p40 (FIG. 35B) in vehicle treated animals and animals treated with varying doses of Compound A in the EAE study.

### DETAILED DESCRIPTION

As used herein, the term “about” has its usual meaning in the context of pharmaceutical and cosmetic formulations to allow for reasonable variations in amounts that can achieve the same effect, typically plus or minus up to 30%. For example, if an amount of “about 1” is provided, then the amount can be up to 1.3 or from 0.70. In cases where “about X” will lead to a figure of above 100%, the term in some embodiments can be read as reflecting up to 100% by weight less the total of the minimum amount of the other ingredients. Likewise, it will be appreciated by one skilled in the art to the extent X is reduced from that upper level the amounts of the other ingredients are increased appropriately. As will be appreciated by one of skill in the art, there is some reasonable flexibility in formulating compositions such that where one or more ingredients are varied, successful formulations can still be made even if an amount falls slightly outside the range. Therefore, to allow for this possibility, amounts are qualified by about. In some embodiments, the examples, e.g., amounts of formulation ingredients can be read as if prefixed with the term “about.” In one or more other embodiments, the examples can be read without the term “about.” In some embodiments, the figures can be read with the term “about.” In one or more other embodiments, the figures can be read without the term “about.” In one or more narrower embodiments, “about” can be plus or minus up to 15% unless the context indicates otherwise. Where “about” is used in connection with “>X” or “<X” or a series of such alternatives, it can, in some embodiments, include about X. Where “about” is used just at the beginning of a series of alternative amounts of “>about X” or “<about X” or “about >X” or “about <X”, it can, in some embodiments, be understood to include “about” before all the other alternatives of the series.

The term Cₘ-Cₙ refers to a group with m to n carbon atoms. For example, the term “C₀” refers to a group with 0 carbon atoms.

The term “alkyl” refers to a monovalent linear or branched saturated hydrocarbon chain. For example, C₁-C₆-alkyl may refer to methyl, ethyl, n-propyl, iso-propyl, n-butyl, sec-butyl, tert-butyl, n-pentyl and n-hexyl. The alkyl groups may be unsubstituted or substituted by one or more substituents.

The term “alkylene” refers to a bivalent linear saturated hydrocarbon chain. For example, C₁-C₃-alkylene may refer to methylene, ethylene, or propylene. The alkylene groups may be unsubstituted or substituted by one or more substituents. For example, the term “C₀-alkylene” refers to a group in which an alkylene chain is absent. For example, “C₀-alkylene-R^{z}” refers to an R^{z} (where R^{z} may refer to any of the R groups mentioned in relation to the formulae described herein). C₀-C₄-alkylene-R^{z} means a group selected from R^{z} and C₁-C₄-alkylene-R^{z}.

The term “haloalkyl” refers to a hydrocarbon chain substituted with at least one halogen atom independently chosen at each occurrence from: fluorine, chlorine, bromine and iodine. The halogen atom may be present at any position on the hydrocarbon chain. For example, C₁-C₆-haloalkyl may refer to chloromethyl, fluoromethyl, trifluoromethyl, chloroethyl e.g., 1-chloromethyl and 2-chloroethyl, trichloroethyl e.g., 1,2,2-trichloroethyl, 2,2,2-trichloroethyl, fluoroethyl e.g., 1-fluoromethyl and 2-fluoroethyl, trifluoroethyl e.g., 1,2,2-trifluoroethyl and 2,2,2-trifluoroethyl, chloropropyl, trichloropropyl, fluoropropyl, trifluoropropyl. A haloalkyl group may be a fluoroalkyl group, i.e., a hydrocarbon chain substituted with at least one fluorine atom. Thus, a haloalkyl group may have any amount of halogen substituents. The group may contain a single halogen substituent, it may have two or three halogen substituents, or it may be saturated with halogen substituents.

The term “alkenyl” refers to a branched or linear hydrocarbon chain containing at least one double bond. The double bond(s) may be present as the E or Z isomer. The double bond may be at any possible position of the hydrocarbon chain. For example, “C₂-C₆-alkenyl” may refer to ethenyl, propenyl, butenyl, butadienyl, pentenyl, pentadienyl, hexenyl and hexadienyl. The alkenyl groups may be unsubstituted or substituted by one or more substituents.

The term “alkynyl” refers to a branched or linear hydrocarbon chain containing at least one triple bond. The triple bond may be at any possible position of the hydrocarbon chain. For example, “C₂-C₆-alkynyl” may refer to ethynyl, propynyl, butynyl, pentynyl and hexynyl. The alkynyl groups may be unsubstituted or substituted by one or more substituents.

The term “cycloalkyl” refers to a saturated hydrocarbon ring system containing 3, 4, 5 or 6 carbon atoms. For example, “C₃-C₆-cycloalkyl” may refer to cyclopropyl, cyclobutyl, cyclopentyl, cyclohexyl. The cycloalkyl groups may be unsubstituted or substituted by one or more substituents.

The term “y- to z-membered heterocycloalkyl” refers to a y- to z-membered heterocycloalkyl group. Thus, it may refer to a monocyclic or bicyclic saturated or partially saturated group having from y to z atoms in the ring system and comprising 1 or 2 heteroatoms independently selected from O, S and N in the ring system (in other words 1 or 2 of the atoms forming the ring system are selected from O, S and N). By partially saturated it is meant that the ring may comprise one or two double bonds. This applies particularly to monocyclic rings with from 5 to 6 members. The double bond will typically be between two carbon atoms but may be between a carbon atom and a nitrogen atom. Examples of heterocycloalkyl groups include: oxirane, aziridine, thirane, oxetane, azetidine, thietane, piperidine, piperazine, morpholine, thiomorpholine, pyrrolidine, tetrahydrofuran, tetrahydrothiophene, dihydrofuran, tetrahydropyran, dihydropyran, dioxane, and azepine. A heterocycloalkyl group may be unsubstituted or substituted by one or more substituents.

Aryl groups may be any aromatic carbocyclic ring system (i.e., a ring system containing 2 (2n+1) π electrons). Aryl groups may have from 6 to 10 carbon atoms in the ring system. Aryl groups will typically be phenyl groups. Aryl groups may be naphthyl groups or biphenyl groups.

The term ‘heterocyclyl’ group refers to rings comprising from 1 to 4 heteroatoms independently selected from O, S and N. The rings may be heterocycloalkyl rings (including both saturated and partially saturated rings) or heteroaryl rings. The term “heterocyclyl” also encompasses groups that are tautomers of hydroxy heteroaryl groups, such pyridones, and tautomers of hydroxy heteroaryl groups that are substituted on the nitrogen, such as N-alkyl pyridones.

The term ‘heterocycloalkenyl’ refers to partially saturated rings comprising from 1 to 2 heteroatoms independently selected from O, S and N.

The term “heteroaryl” refers to any aromatic (i.e., a ring system containing 2 (2n+1) π electrons) 5 or 6 membered ring system comprising from 1 to 4 heteroatoms independently selected from O, S and N (in other words from 1 to 4 of the atoms forming the ring system are selected from O, S and N). Thus, any heteroaryl groups may be independently selected from: 5 membered heteroaryl groups in which the heteroaromatic ring is substituted with 1-4 heteroatoms independently selected from O, S and N; and 6-membered heteroaryl groups in which the heteroaromatic ring is substituted with 1-3 (e.g., 1-2) nitrogen atoms. Specifically, heteroaryl groups may be independently selected from: pyrrole, furan, thiophene, pyrazole, imidazole, oxazole, isoxazole, triazole, oxadiazole, thiadiazole, tetrazole; pyridine, pyridazine, pyrimidine, pyrazine, triazine.

For variables which may be selected from “carbon” and “nitrogen” (i.e., X¹, X², X³, X⁴, X⁵, etc.), it is understood that the carbon or nitrogen may additionally comprise hydrogen and/or a designated substituent to the ring system (i.e., —R^{2a}, R⁴).

On ring systems designating an optional substituent (i.e., —R^{2a}, R⁴), it is understood that the substituent, if present, may replace a hydrogen on any carbon or nitrogen of the ring system.

Compounds of the disclosure containing one or more asymmetric carbon atoms can exist as two or more stereoisomers. Certain compounds of the disclosure may exist in particular geometric and/or stereoisomeric forms and the present disclosure contemplates all such compounds, including cis- and trans-isomers, R- and S-enantiomers, diastereomers, the racemic mixtures thereof, and other mixtures thereof, as falling within the scope of the disclosure. Additional asymmetric carbon atoms may be present in a substituent such as an alkyl group. All such isomers, as well as mixtures thereof, are included in this disclosure.

Where a compound of the disclosure contains a double bond such as a C═C or C═N group, geometric cis/trans (or Z/E) isomers are possible. Where structural isomers are interconvertible via a low energy barrier, tautomeric isomerism (‘tautomerism’) can occur. This can take the form of proton tautomerism in compounds of the disclosure containing, for example, an imino, keto, or oxime group, or so-called valence tautomerism in compounds which contain an aromatic moiety. It follows that a single compound may exhibit more than one type of isomerism.

Included within the scope of the present disclosure are all stereoisomers, geometric isomers and tautomeric forms of the compounds of the disclosure, including compounds exhibiting more than one type of isomerism, and mixtures of one or more thereof. Also included are acid addition or base salts wherein the counter ion is optically active, for example, d-lactate or I-lysine, or racemic, for example, dl-tartrate or dl-arginine.

Cis/trans isomers may be separated by conventional techniques well known to those skilled in the art, for example, chromatography and fractional crystallisation.

Conventional techniques for the preparation/isolation of individual enantiomers, when necessary, include chiral synthesis from a suitable optically pure precursor or resolution of the racemate (or the racemate of a salt or derivative) using, for example, chiral high pressure liquid chromatography (HPLC). Thus, chiral compounds of the disclosure (and chiral precursors thereof) may be obtained in enantiomerically-enriched form using chromatography, typically HPLC, on an asymmetric resin with a mobile phase consisting of a hydrocarbon, typically heptane or hexane, containing from about 0 to about 50% by volume of isopropanol, typically from about 2% to about 20%, and for specific examples, about 0 to about 5% by volume of an alkylamine e.g., about 0.1% diethylamine. Concentration of the eluate affords the enriched mixture.

Alternatively, the racemate (or a racemic precursor) may be reacted with a suitable optically active compound, for example, an alcohol, or, in the case where the compound of the disclosure contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, “Stereochemistry of Organic Compounds” by E. L. Eliel and S. H. Wilen (Wiley, 1994).

The present disclosure also includes all pharmaceutically acceptable isotopically-labelled compounds of the formulae described herein and their syntheses, wherein one or more atoms are replaced by atoms having the same atomic number, but an atomic mass or mass number different from the atomic mass or mass number usually found in nature.

Examples of isotopes suitable for inclusion in the compounds of the disclosure include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Isotopically-labelled compounds can generally be prepared by conventional techniques known to those skilled in the art or by processes analogous to those described using an appropriate isotopically-labelled reagent in place of the non-labelled reagent previously employed.

Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic, and valeric acids. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulfate and hemicalcium salts.

The activity of the compounds of the present disclosure can be assessed by a variety of in silico, in vitro and in vivo assays. In silico analysis of a variety of compounds has been demonstrated to be predictive of ultimate in vitro and even in vivo activity.

The term “composition” as used herein is intended to include or encompass a product comprising specified ingredients in predetermined amounts or proportions, as well as any product which results, directly or indirectly, from combination of the specified ingredients in the specified amounts. This term in relation to pharmaceutical compositions is intended to encompass a product comprising one or more active ingredients, and an optional pharmaceutically acceptable carrier comprising inert ingredients, as well as any product which results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. In the pharmaceutical composition, the active object compound is included in an amount sufficient to produce the desired effect upon the process or condition of diseases.

Accordingly, the pharmaceutical compositions of the present invention encompass any composition made by admixing a compound of the present invention and a pharmaceutically acceptable carrier. Said compositions are prepared according to conventional mixing, granulating, or coating methods, respectively, and contain a therapeutically effective amount of the active ingredient.

By “pharmaceutically acceptable” it is meant the carrier, diluent or excipient must be compatible with the other ingredients of the formulation and not deleterious to the recipient thereof. Pharmaceutical compositions intended for oral use may be prepared according to any method known to the art for the manufacture of pharmaceutical compositions and such compositions may contain one or more agents selected from the group consisting of sweetening agents, flavoring agents, coloring agents, and preserving agents in order to provide pharmaceutically elegant and palatable preparations.

The compositions of the present invention may be presented in unit dosage form and may be prepared by any of the methods well known in the art of pharmacy. The term “unit dosage form” is taken to mean a single dose wherein all active and inactive ingredients are combined in a suitable system, such that the patient or person administering the drug to the patient can open a single container or package with the entire dose contained therein and does not have to mix any components together from two or more containers or packages. Typical examples of unit dosage forms are tablets or capsules for oral administration. These examples of unit dosage forms are not intended to be limiting in any way, but merely to represent typical examples in the pharmacy arts of unit dosage forms.

As used herein, the term “appendage” includes a hand, a foot, a wrist, an ankle, and/or a joint.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e. arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” includes the amount of a compound that, when administered to a mammal for treating a disease, is sufficient to affect such treatment for the disease. The “therapeutically effective amount” will vary depending on the compound, the disease and its severity and the age, weight, etc., of the mammal to be treated.

A compound of the disclosure, or pharmaceutically acceptable salt thereof, may be used on its own or may be administered in the form of a pharmaceutical composition in which the compounds of the disclosure, or a pharmaceutically acceptable salt thereof, is in association with a pharmaceutically acceptable adjuvant, diluent or carrier.

Conventional procedures for the selection and preparation of suitable pharmaceutical formulations are described in, for example, “Pharmaceuticals—The Science of Dosage Form Designs”, M. E. Aulton, Churchill Livingstone, 1988. Examples of topical formulations and dosage forms are described in Remington: The Science and Practice of Pharmacy (21^{st} Edition), University of the Sciences in Philadelphia. Further examples of formulations are provided in the Examples herein. In some embodiments, formulations for the compounds disclosed herein include a 20% propylene glycol/20% Vitamin E TPGS/60% water formulation; a HPPCD (20% solution (w/v)/DMSO (99/1))-based formulation; a 1% methylcellulose formulation; a 5% DMSO/40% PEG-400/55% Milli-Q Water formulation; a 5% DMSO/15% PEG-400/80% (10% Vitamin E TPGS in water) formulation; a 5% DMSO/40% PEG-400, 55% water (0.9% NaCl) formulation; and a 5% DMSO/15% PEG-400/80% E-TPGS in purified water formulation.

Depending on the mode of administration of the compounds of the disclosure, the pharmaceutical composition which is used to administer the compounds of the disclosure will, in some embodiments comprise from about 0.005 to about 99% w/w compounds of the disclosure, or comprise from about 0.05 to about 80% w/w compounds of the disclosure, or comprise from about 0.10 to about 70% w/w compounds of the disclosure, or comprise from about 0.10 to about 50% w/w compounds of the disclosure (all percentages by weight being based on total composition). In some embodiments, the pharmaceutical composition which is used to administer the compounds of the disclosure will comprise from about 0.005 to about 40% w/w compounds of the disclosure, or comprise from about 0.005 to about 30% w/w compounds of the disclosure, or comprise from about 0.010 to about 20% w/w compounds of the disclosure, or comprise from about 0.010 to about 10% w/w compounds of the disclosure or comprise from about 0.005 to about 5% w/w compounds of the disclosure, or comprise from about 0.005 to about 2% w/w compounds of the disclosure, or comprise from about 0.005 to about 1% w/w compounds of the disclosure, or comprise from about 0.005 to about 0.5% w/w compounds of the disclosure, or comprise from about 0.010 to about 1% w/w compounds of the disclosure, or comprise from about 0.010 to about 0.5% w/w compounds of the disclosure. In some embodiments, the pharmaceutical composition which is used to administer the compounds of the disclosure will comprise from about 0.010 to about 0.3% w/w compounds of the disclosure, In some embodiments, the pharmaceutical composition will comprise about 0.01% w/w, or about 0.02% w/w, or about 0.03% w/w, or about 0.05% w/w, about 0.075% w/w, or about 0.1% w/w, about 0.2% w/w, or about 0.3% w/w about 0.4% w/w, or about 0.5% w/w (all percentages by weight being based on total composition).

The pharmaceutical compositions may be administered topically (e.g., to the skin) in the form, e.g., of creams, ointments, gels, lotions, solutions, suspensions; or systemically, e.g., by oral administration in the form of tablets, lozenges, hard or soft capsules, solutions, aqueous or oily suspensions, emulsions, dispersible powders or granules, syrups or elixirs; or by parenteral administration in the form of a sterile aqueous or oily solution, suspension or emulsion for injection (including intraarticular, intravenous, intracoronary, subcutaneous, intramyocardial, intraperitoneal, intramuscular, intravascular or infusion); by rectal administration in the form of suppositories or enemas; by inhalation, for example, as a finely divided powder or a liquid aerosol or mist; or for administration by insufflation (for example as a finely divided powder).

For oral administration, the compounds of the disclosure may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent. Thus, compositions intended for oral use may contain, for example, one or more colouring, sweetening, flavouring and/or preservative agents.

For the preparation of soft gelatine capsules, the compounds of the disclosure may be admixed with, for example, a vegetable oil or polyethylene glycol. Hard gelatine capsules may contain granules of the compound using either of the above-mentioned excipients for tablets. Also, liquid or semisolid formulations of the compound of the disclosure may be filled into hard gelatine capsules. Liquid preparations for oral application may be in the form of syrups or suspensions, for example, solutions containing the compound of the disclosure, the balance being sugar and a mixture of ethanol, water, glycerol and propylene glycol. Optionally such liquid preparations may contain colouring agents, flavouring agents, sweetening agents (such as saccharine), preservative agents and/or carboxymethylcellulose as a thickening agent or other excipients known to those skilled in art.

For intravenous (parenteral) administration, the compounds of the disclosure may be administered as a sterile aqueous/hydrophilic or hydrophobic/oily solution or suspension.

The size of the dose for therapeutic or prophylactic purposes of a compound of the disclosure will naturally vary according to the nature and severity of the conditions, the concentration of the compound required for effectiveness in isolated cells, the concentration of the compound required for effectiveness in experimental animals, the age and sex of the animal or patient, and the route of administration, according to well-known principles of medicine.

Dosage levels, dose frequency, and treatment durations of compounds of the disclosure are expected to differ depending on the formulation and clinical indication, age, and co-morbid medical conditions of the patient.

An effective amount of a compound of the present disclosure for use in therapy of a condition is an amount sufficient to achieve symptomatic relief in a warm-blooded animal, particularly a human, of the symptoms of the condition, to mitigate the physical manifestations of the condition, or to slow the progression of the condition.

The compositions described herein may be presented in unit dosage form and may be prepared by any of the methods well known in the art of pharmacy. The term “unit dosage form” is taken to mean a single dose wherein all active and inactive ingredients are combined in a suitable system, such that the patient or person administering the drug to the patient can open a single container or package with the entire dose contained therein and does not have to mix any components together from two or more containers or packages. Typical examples of unit dosage forms are tablets or capsules for oral administration. These examples of unit dosage forms are not intended to be limiting in any way, but merely to represent typical examples in the pharmacy arts of unit dosage forms.

The concentration of the compounds disclosed herein in a formulation can vary a great deal, and will depend on a variety of factors, including the disease or condition to be treated, the selectivity, potency and bioavailability of the compounds disclosed herein, the desired effect, possible adverse reactions, the ability and speed of the compounds disclosed herein to reach its intended target, and other factors within the particular knowledge of the patient and physician.

The amount of active ingredient that is combined with one or more excipients to produce a single dosage form will necessarily vary depending upon the host treated and the particular route of administration. For example, a formulation intended for oral administration to humans will generally contain, for example, from about 0.5 mg to about 0.5 g of active agent (more suitably from about 0.5 mg to about 100 mg, for example from about 1 mg to about 30 mg) compounded with an appropriate and convenient amount of excipients which may vary from about 5 to about 98 or about 99 percent by weight of the total composition.

For the above-mentioned compounds of the disclosure, the dosage administered will, of course, vary with the compound employed, the mode of administration, the treatment desired and the disorder indicated. In using a compound of the disclosure for therapeutic or prophylactic purposes it will generally be administered so that a daily dose in the range, for example, a daily dose selected from about 0.1 mg/kg to about 100 mg/kg, about 1 mg/kg to about 75 mg/kg, about 1 mg/kg to about 50 mg/kg, about 1 mg/kg to about 20 mg/kg or about 5 mg/kg to about 10 mg/kg body weight is received, given if required in divided doses. In general, lower doses will be administered when a parenteral route is employed. Thus, for example, for intravenous or intraperitoneal administration, a dose in the range, for example, about 0.1 mg/kg to about 30 mg/kg body weight will generally be used. Similarly, for administration by intraarticular, a dose in the range, for example, about 0.01 mg/kg to about 30 mg/kg body weight may generally be used. For administration by inhalation, a dose in the range, for example, about 0.05 mg/kg to about 25 mg/kg body weight will be used. Suitably the compound of the disclosure is administered orally, for example in the form of a tablet, or capsule dosage form. The daily dose administered orally may be, for example a total daily dose selected from about 1 mg to about 1000 mg, about 5 mg to about 1000 mg, about 10 mg to about 750 mg or about 25 mg to about 500 mg. Typically, unit dosage forms will contain about 0.5 mg to about 0.5 g of a compound of this disclosure. In some embodiments, the dose will be about 0.1 mg/kg to about 10 mg/kg, or about 0.1 mg/kg to 1 mg/kg, or about 0.1 mg/kg to about 2 mg/kg, or about 1 mg/kg to 10 mg/kg by body weight. In some embodiments, the dose will be about 0.1 mg/kg, or about 0.15 mg/kg, or about 0.3 mg/kg, or about 0.5 mg/kg, or about 0.8 mg/kg, or about 1 mg/kg, or about 3 mg/kg, or about 6 mg/kg, or about 10 mg/kg by body weight. In some embodiments, where the compound is more active and/or has a higher bioavailability, a lower unit dosage amount may be appropriate, e.g., about 0.2 mg to about 0.2 g of a compound of this disclosure. In some embodiments, the compounds disclosed herein are administered daily. In some embodiments, the compounds disclosed herein are administered on alternate days. In some embodiments, the compounds disclosed herein are administered once a week. The period of administration in some embodiments, is for a number of days, in some embodiments, is for about one to twelve weeks, and in some embodiments, is for about three to twelve months.

The compounds of the disclosure may be administered along with other active compounds as part of a treatment regime. The other active compounds may be administered simultaneously with, subsequently to or previously to the administration of the compounds of the disclosure. It may be that the pharmaceutical formulation comprising the compounds of the disclosure also comprises one or more other active compounds. The other active compounds may be anticancer, anti-inflammatory, antibacterial, antiviral, antiemetic, antithrombotic, or compounds that alter the metabolism.

Throughout the description and claims of this specification, the words “comprise” and “contain” and variations of them mean “including but not limited to”, and they are not intended to (and do not) exclude other moieties, additives, components, integers, or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment, or example of the disclosure are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The disclosure is not restricted to the details of any foregoing embodiments.

The disclosure extends to any novel one, or any novel combination, of the features disclosed in this specification (including any accompanying claims, abstract and drawings), or to any novel one, or any novel combination, of the steps of any method or process so disclosed.

Embodiments disclosed herein are independent and interchangeable. Any one embodiment may be combined with any other embodiment, where chemically allowed and/or biologically appropriate, as would be understood by a person of ordinary skill in the art. In other words, any of the features described in the embodiments may (where chemically allowable and/or biologically appropriate) be combined with the features described in one or more other embodiments. For example, where a compound or a disease or disorder is exemplified or illustrated in this specification, any two or more of the embodiments listed herein, expressed at any level of generality, which encompass that compound or disease or disorder may be combined to provide a further embodiment which forms part of the present disclosure. For the avoidance of doubt, terms encompassing a range of formulae, such as “any of formulae (XII)—(XX),” encompass sub-formulae therein, for example formula (XVIIA).

It will be understood that various modifications may be made to the embodiments disclosed herein. Therefore, the above description should not be construed as limiting, but merely as exemplifications of preferred embodiments. For example, the functions described above and implemented as the best mode for operating the present disclosure are for illustration purposes only. Other arrangements and methods may be implemented by those skilled in the art without departing from the scope and spirit of this disclosure.

Moreover, those skilled in the art will envision other modifications within the scope and spirit of the specification appended hereto.

The reader's attention is directed to all papers and documents which are filed concurrently with or previous to this specification in connection with this application and which are open to public inspection with this specification, and the contents of all such papers and documents are incorporated herein by reference.

In some embodiments, the present disclosure provides a compound of formula (1), or a pharmaceutically acceptable salt or N-oxide thereof:

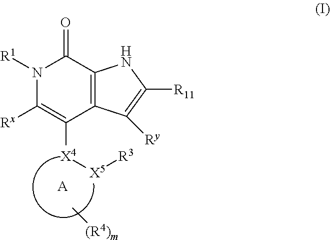

wherein:
Ring A is independently selected from phenyl, 5-membered heterocyclyl, and 6-membered heterocyclyl, wherein X⁴ and X⁵ are independently selected from carbon and nitrogen:
R¹ is independently selected from C₁-C₅-alkyl, C₁-C₅-haloalkyl, C₂-C₆-alkynyl, COR⁶, CO₂R⁶, C₁-C₄-alkylene-NR⁵R⁶, C₁-C₄-alkylene-OR⁷, C₁-C₄-alkyl-S(O)₂R⁶, C₃-C₆-cycloalkyl, aryl, heteroaryl, and 3- to 6-membered heterocycloalkyl;
R² is absent or independently selected from H, halo, cyano, nitro, SF₄, SF₅, ═O, S(O)₂R⁶, alkoxy, C₁-C₆-haloalkyl, C₁-C₈-alkyl, C₃-C₆-cycloalkyl, 4- to 8-membered heterocycloalkenyl, 3- to 8-membered heterocycloalkyl, aryl, and 5- or 6-membered heteroaryl,
   wherein the C₁-C₈-alkyl, alkoxy, C₃-C₆-cycloalkyl, 4- to 8-membered heterocycloalkenyl, 3- to 8-membered heterocycloalkyl, aryl, and 5- or 6-membered heteroaryl are independently optionally substituted with one or more groups selected from halogen, cyano, nitro, hydroxy, SF₅, amide, ester, alkoxy, and C₁-C₄-alkyl;
R³ is independently selected from R^{3a} and OR^{3b};
R^{3a} is independently selected from H, CN, C₁-C₄-alkyl, C₂-C₄-alkenyl, C₂-C₄-alkynyl, C₁-C₄-haloalkyl, C₂-C₄-haloalkenyl, C₃-C₃-cycloalkyl, C₅-C₃-cycloalkenyl, 5- to 9-membered heterocycloalkenyl, 3- to 9-membered heterocycloalkyl, phenyl, and 5- to 9-membered heteroaryl;
   wherein the cycloalkyl, heterocycloalkyl, cycloalkenyl, and heterocycloalkenyl are independently optionally substituted with from 1 to 4 R⁸ groups; and
   wherein the phenyl and the 5- to 9-membered heteroaryl are independently optionally substituted with from 1 to 5 R⁹ groups;
R^{3b} is independently selected from C₁-C₄-alkyl, C₂-C₄-alkylene-O—C₁-C₄-alkyl, C₁-C₄-haloalkyl, C₃-C₈-cycloalkyl, 3- to 8-membered heterocycloalkyl, phenyl, and 5- or 6-membered heteroaryl;
   wherein the cycloalkyl and 3- to 8-membered heterocycloalkyl are independently optionally substituted with from 1 to 4 R⁸ groups; and
   wherein the phenyl and the 5- or 6-membered heteroaryl are independently optionally substituted with from 1 to 5 R⁹ groups;
R⁴ is independently at each occurrence selected from H, ═O, ═S, halo, nitro, cyano, C₀-C₄-alkylene-NR⁵R⁶, —NR⁵R⁶, C₀-C₄-alkylene-OR⁷, —OR⁷, SR⁶, SOR⁶, C₀-C₄-alkylene-S(O)₂R⁶, —S(O)₂R⁶, SO₂NR⁶R⁶, C₀-C₄-alkylene-CO₂R⁶, —CO₂R⁶, C₀-C₄-alkylene-C(O)R⁶, —C(O)R⁶, C₀-C₄-alkylene-CONR⁶R⁶, —CONR⁶R⁶, C₁-C₆-alkyl, C₁-C₄-alkyl-S(O)₂R⁶, C₂-C₄-alkenyl, C₂-C₄-alkynyl, C₁-C₆-haloalkyl, C₃-C₆-cycloalkyl, aryl, 4- to 6-membered heterocycloalkyl, and 5- or 6-membered heteroaryl;
   wherein the aryl, 4- to 6-membered heterocycloalkyl, and 5- or 6-membered heteroaryl are independently optionally substituted with from 1 to 5 R⁹ groups;
R⁵ is independently at each occurrence selected from H, C₁-C₄-alkyl, C(O)—C₁-C₄-alkyl, and S(O)₂—C₁-C₄-alkyl; or R⁵ and R⁶, together with the nitrogen atom to which they are attached, form a C₅-C₈-heterocycloalkyl group optionally substituted with from 1 to 4 R⁸ groups;
R⁶ is independently at each occurrence selected from H and C₁-C₆-alkyl; or where two R⁶ groups are attached to the same nitrogen, those two R⁶ groups, together with the nitrogen atom to which they are attached, optionally form a C₅-C₈-heterocycloalkyl group optionally substituted with from 1 to 4 R⁸ groups;
R⁷ is independently at each occurrence selected from H, C₁-C₄-alkyl, C(O)—C₁-C₄-alkyl, and C₁-C₄-haloalkyl;
R⁸ is independently at each occurrence selected from ═O, ═S, fluoro, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl, 4- to 6-membered heterocycloalkyl, and C₃-C₆-cycloalkyl;
R⁹ is independently at each occurrence selected from halo, nitro, cyano, NR⁵R⁶, C₁-C₄-alkyl-OR⁷, OR⁷, SR⁶, SOR⁶, C₁-C₄-alkyl-S(O)₂R⁶, S(O)₂R⁶, SO₂NR⁶R⁶, CO₂R⁶, C(O)R⁶, CONR⁶R⁶, C₀-C₄-alkylene-R^{9a}, C₁-C₄-alkyl, C₂-C₄-alkenyl, C₂-C₄-alkynyl, C₁-C₄-haloalkyl, 4- to 6-membered heterocycloalkyl, and C₃-C₆-cycloalkyl; or where two R⁹ groups are attached to adjacent atoms, those two R⁹ groups, together with the atoms to which they are attached, optionally form a 5- or 6-membered heterocycloalkyl group optionally substituted with from 1 to 4 R⁸ groups:
R^{9a} is independently at each occurrence selected from 4- to 6-membered heterocycloalkyl;
R¹⁰ is absent or is independently at each occurrence selected from H, halo, C₁-C₆-alkyl, C₁-C₆-haloalkyl, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₆-alkyl, C₂-C₆-alkenyl, C₂-C₆-alkynyl and 3- to 8-membered heterocycloalkyl;
R¹¹ is independently selected from phenyl, heteroaryl, and heterocyclyl, each optionally substituted with 1 to 4 R² groups and 1 to 3 R¹⁰ groups;
R^{x} and R^{y} are each independently selected from H, halo, nitro, cyano, C₁-C₆-alkylene-NR⁵R⁶, NR⁵R⁶, C₁-C₆-alkylene-OR⁷, C₁-C₆-alkyl-OR⁷, OR⁷, C₁-C₆-alkyl-SR⁶, SR⁶, C₁-C₆-alkyl-SOR⁶, SOR³, C₁-C₆-alkyl-S(O)₂R⁶, S(O)₂R⁶, C₁-C₆-alkyl-SO₂NR⁶R⁶, SO₂NR³R⁶, C₁-C₆-alkyl-CO₂R⁶, CO₂R⁶, C₁-C₆-alkyl-C(O)R⁶, C(O)R⁶, C₁-C₆-alkyl-CONR⁶R⁶, CONR⁶R⁶, C₁-C₆-alkyl, C₂-C₆-alkenyl, C₂-C₆-alkynyl, C₁-C₆-haloalkyl, C₃-C₈-cycloalkyl, 3- to 8-membered heterocycloalkyl, aryl, aryloxy, and 5- to 8-membered heteroaryl;
m is an integer selected from 0, 1, 2, 3 and 4;
wherein any of the aforementioned alkyl, alkylene, alkenyl, or C₃-C₆-cycloalkyl groups is optionally substituted, where chemically possible, by 1 to 5 substituents which are each independently at each occurrence selected from the group consisting of: C₁-C₄-alkyl, oxo, fluoro, nitro, cyano, NR^{a}R^{b}, OR^{a}, SR^{a}, CO₂R^{a}, C(O)R^{a}, CONR^{a}R^{a}, S(O)R^{a} and S(O)₂R^{a};
wherein R^{a} is independently at each occurrence selected from H, C₁-C₄-alkyl and C₁-C₄-haloalkyl; and
wherein R^{b} is independently at each occurrence selected from H, C₁-C₄-alkyl, C(O)—C₁-C₄-alkyl and S(O)₂—C₁-C₄-alkyl.

In embodiment A, the present disclosure provides a compound of formula (I), or a pharmaceutically acceptable salt or N-oxide thereof:

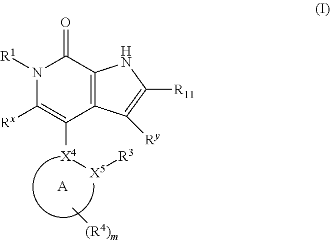

wherein:
Ring A is independently selected from phenyl, 5-membered heterocyclyl and 6-membered heterocyclyl, wherein X⁴ is independently selected from carbon and nitrogen and X⁵ is independently selected from carbon and nitrogen;
R¹ is independently selected from C₁-C₃-alkyl, C₁-C₃-fluoroalkyl, C₃-C₄-cycloalkyl and 4-membered heterocycloalkyl;
R¹¹ is independently selected from 5-membered heterocyclyl, 6-membered heterocyclyl and phenyl, each optionally substituted with from 1 to 4 R^{2a} groups;
R^{2a} is independently at each occurrence selected from ═O, ═S, halo, nitro, cyano, SF₆, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl, C₃-C₆ cycloalkyl and 4- to 6-membered heterocyclyl;
R³ is independently selected from R^{3a}, OR^{3b}, and NR⁶R^{3b};
R^{3a} is independently selected from H, CN, C₁-C₄-alkyl, C₂-C₄-alkenyl, C₂-C₄-alkynyl, C₁-C₄-haloalkyl, C₂-C₄-haloalkenyl, and C₀-C₃-alkylene-R^{3c}; wherein R^{3c} is independently at each occurrence selected from C₃-C₈-cycloalkyl, C₅-C₈-cycloalkenyl, 5- to 8-membered heterocycloalkenyl, 3- to 8-membered heterocycloalkyl, phenyl and 5- or 6-membered heteroaryl; wherein where R^{3c} is cycloalkyl, heterocycloalkyl, cycloalkenyl, or heterocycloalkenyl, R^{3c} is optionally substituted with from 1 to 4 R⁸ groups and where R^{3c} is phenyl or heteroaryl, R^{3c} is optionally substituted with from 1 to 5 R⁹ groups:
R^{3b} is independently selected from C₁-C₄-alkyl, C₂-C₄-alkylene-O—C₁-C₄-alkyl, C₁-C₄-haloalkyl and C₀-C₃-alkylene-R^{3d}; wherein R^{3d} is independently at each occurrence selected from C₃-C₈-cycloalkyl, 3- to 8-membered heterocycloalkyl, phenyl and 5- or 6-membered heteroaryl; wherein where R^{3d} is cycloalkyl or heterocycloalkyl, R^{3d} is optionally substituted with from 1 to 4 R⁸ groups and where R^{3d} is phenyl or heteroaryl, R^{3d} is optionally substituted with from 1 to 5 R⁹ groups;
R⁴ is independently at each occurrence selected from ═O, ═S, halo, nitro, cyano, C₀-C₄-alkylene-NR⁵R⁶, C₀-C₄-alkylene-OR⁷, SR⁶, SOR⁶, C₀-C₄-alkylene-S(O)₂R⁶, SO₂NR⁶R⁶, C₀-C₄-alkylene-CO₂R⁶, C₀-C₄-alkylene-C(O)R⁶, C₀-C₄-alkylene-CONR⁶R⁶, C₁-C₄-alkyl, C₁-C₄-alkyl-S(O)₂R⁶, C₂-C₄-alkenyl, C₂-C₄-alkynyl, C₁-C₄-haloalkyl, C₃-C₆-cycloalkyl, and C₀-C₄-alkylene-R^{4c};
R^{4c} is independently at each occurrence selected from C₃-C₆-cycloalkyl and 4- to 6-membered heterocycloalkyl;
R⁵ is independently at each occurrence selected from H, C₁-C₄-alkyl, C(O)—C₁-C₄-alkyl and S(O)₂—C₁-C₄-alkyl; or R⁵ and R⁶, together with the nitrogen atom to which they are attached form a C₅-C₈-heterocycloalkyl group optionally substituted with from 1 to 4 R⁸ groups;
R⁶ is independently at each occurrence selected from H and C₁-C₄-alkyl; or where two R⁶ groups are attached to the same nitrogen, those two R⁶ groups together with the nitrogen atom to which they are attached optionally form a C₅-C₈-heterocycloalkyl group optionally substituted with from 1 to 4 R⁸ groups;
R⁷ is independently at each occurrence selected from H, C₁-C₄-alkyl, C(O)—C₁-C₄-alkyl and C₁-C₄-haloalkyl;
R⁸ is independently at each occurrence selected from ═O, ═S, fluoro, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl and cyclopropyl;
R⁹ is independently at each occurrence selected from halo, nitro, cyano, C₀-C₄-alkylene-NR⁵R⁶, C₀-C₄-alkylene-OR^{7a}, C₀-C₄-alkylene-SR⁶, C₀-C₄-alkylene-SOR⁶, C₀-C₄-alkylene-S(O)₂R⁶, C₀-C₄-alkylene-SO₂NR⁶R⁶, C₀-C₄-alkylene-CO₂R⁶, C₀-C₄-alkylene-C(O)R⁶, C₀-C₄-alkylene-CONR⁶R⁶, C₀-C₄-alkylene-R^{9a}, C₁-C₄-alkyl, C₂-C₄-alkenyl, C₂-C₄-alkynyl, C₁-C₄-haloalkyl; or where two R⁹ groups are attached to adjacent atoms, those two R⁹ groups together with the atoms to which they are attached optionally form a 5- or 6-membered heterocycloalkyl group optionally substituted with from 1 to 4 R⁸ groups;
R^{7a} is independently at each occurrence selected from H, C₁-C₄-alkyl, C(O)—C₁-C₄-alkyl, C₀-C₄-alkylene-NR₅R₆, —C₀-C₄-alkyl-O—R⁷, C₀-C₄-alkylene-SR⁶, C₀-C₄-alkylene-SOR⁶, C₀-C₄-alkylene-S(O)₂R⁶, C₀-C₄-alkylene-SO₂NR⁶R⁶, C₀-C₄-alkylene-CO₂R⁶, C₀-C₄-alkylene-C(O)R⁶, C₀-C₄-alkylene-CONR⁶R⁶ and C₁-C₄-haloalkyl;
R^{9a} is independently at each occurrence selected from C₃-C₆-cycloalkyl and 4- to 6-membered heterocycloalkyl;
R^{x} and R^{y} are each independently selected from H, halo, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl, C₃-C₄-cycloalkyl and 4-membered heterocycloalkyl;
m is an integer selected from 0, 1, 2, 3 and 4;
wherein any of the aforementioned alkyl, alkylene, alkenyl, cycloalkyl or heterocycloalkyl groups is optionally substituted, where chemically possible, by 1 to 5 substituents which are each independently at each occurrence selected from the group consisting of: C₁-C₄-alkyl, oxo, fluoro, nitro, cyano, NR^{a}R^{b}, OR^{a}, SR^{a}, CO₂R^{a}, C(O)R^{a}, CONR^{a}R^{a}, S(O)R^{a} and S(O)₂R^{a}; wherein R^{a} is independently at each occurrence selected from H, and C₁-C₄-alkyl; and R^{b} is independently at each occurrence selected from H, C₁-C₄-alkyl, C(O)—C₁-C₄-alkyl and S(O)₂—C₁-C₄-alkyl. It may be that R¹¹ is selected from pyrazolyl and imidazolyl, each optionally substituted with from 1 to 4 R^{2a} groups.

In one or more embodiments, the compounds of formula (I) may be an enantiomer, a mixture of enantiomers, a racemate, a diastereoisomer, a mixture of diastereoisomers, a geometric isomer, a mixture of geometric isomers, a tautomer, or a mixture of tautomers. The compounds of formula (I) may also be in the form of a solvate or hydrate.

The embodiments disclosed herein apply to compounds of formula (I) (as described in embodiment A above). These embodiments are independent and interchangeable. Any one embodiment may be combined with any other embodiment, where chemically allowed. In other words, any of the features described in the following embodiments may (where chemically allowable) be combined with the features described in one or more other embodiments. In particular, where a compound is exemplified or illustrated in this specification, any two or more of the embodiments listed below, expressed at any level of generality, which encompass that compound may be combined to provide a further embodiment which forms part of the present disclosure.

It may be that R¹¹ is selected from pyrazolyl and imidazolyl, each optionally substituted with from 1 to 4 R^{2a} groups.

In some embodiments, R¹¹ is

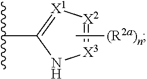

wherein
X¹, X² and X³ are each independently selected from carbon and nitrogen, wherein only one of X¹, X² and X³ are nitrogen and the other two of X¹, X² and X³ are carbon; and
n is independently an integer selected from 0, 1, 2, 3 and 4.

In some embodiments, R¹¹ is

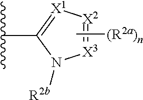

wherein R^{2b} is independently selected from H, C₁-C₄-alkyl, C₃-C₆ cycloalkyl, and 4- to 6-membered heterocyclyl.

In some embodiments, X¹ is carbon. In some embodiments, X¹ is nitrogen.

In some embodiments, X² and X³ are each independently selected from carbon and nitrogen. In some embodiments, X² and X³ are each carbon.

In some embodiments, R¹¹ is a 5-membered heterocyclyl group; optionally substituted with from 1 to 4 R^{2a} groups.

In some embodiments, R¹¹ is

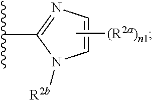

wherein n1 is independently an integer selected from 0, 1 and 2.

In some embodiments, R¹¹ is

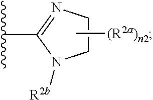

wherein n2 is independently an integer selected from 0, 1, 2 and 3.

In some embodiments, R¹¹ is

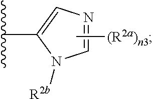

wherein n3 is independently an integer selected from 0, 1 and 2.

In some embodiments, R¹¹ is

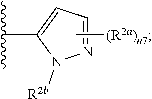

wherein n7 is independently an integer selected from 0, 1, and 2.

In some embodiments, 1R¹¹ is

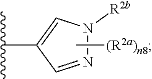

wherein n8 is independently an integer selected from 0, 1, and 2.

R^{2b} is independently selected from H, C₁-C₄-alkyl, C₃-C₆ cycloalkyl, and 4- to 6-membered heterocyclyl. R^{2b} may be H. R^{2b} may be selected from H, C₁-C₄-alkyl and cyclopropyl. R^{2b} may be selected from C₁-C₄-alkyl and cyclopropyl. R^{2b} may be C₁-C₄-alkyl, e.g., methyl. R^{2b} may be 4- to 6-membered heterocyclyl. R^{2b} may be oxetanyl or azetidinyl. In some embodiments, R^{2b} is oxetanyl. In some embodiments, R^{2b} is oxetan-3-yl.

In some embodiments, R¹¹ is

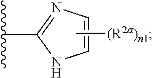

wherein n1 is independently an integer selected from 0, 1 and 2.

In some embodiments, R¹¹ is

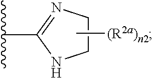

wherein n2 is independently an integer selected from 0, 1, 2 and 3.

In some embodiments, R¹ is

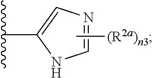

wherein n3 is independently an integer selected from 0, 1 and 2.

In some embodiments, R¹¹ is

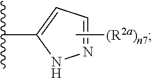

wherein n7 is independently an integer selected from 0, 1, 2, and 3.

In some embodiments, R¹¹ is

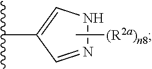

wherein n8 is independently an integer selected from 0, 1, 2, and 3. In an embodiment R^{2a} is not C₁-C₄-alkyl. In an embodiment R^{2a} is not methyl.

In some embodiments, R¹¹ is a substituted or unsubstituted imidazolidine or a substituted or unsubstituted imidazoline.

In embodiments where R¹¹ is depicted as comprising an NH group within the ring, it is to be understood that the nitrogen atom may be substituted with an R^{2a} group defined herein, where chemically possible, to give an NR^{2a} group within the ring.

In some embodiments, R^{2a} is independently at each occurrence selected from ═O, halo, nitro, cyano, NR⁵R⁶, OR⁷, SR⁶, S(O)₂R⁶, SO₂NR⁶R⁶, CO₂R⁶, C(O)R⁶, CONR⁶R⁶, C₁-C₄-alkyl, C₁-C₄-haloalkyl, C₃-C₆ cycloalkyl, and 4- to 6-membered heterocyclyl.

In some embodiments, R^{2a} is independently at each occurrence selected from ═O, halo, cyano, CO₂R⁶, C(O)R⁶, C₁-C₄-alkyl, C₁-C₄-haloalkyl, C₃-C₆ cycloalkyl, and 4- to 6-membered heterocyclyl.

In some embodiments, R^{2a} is independently at each occurrence selected from ═O, halo, cyano, S(O)₂R⁶, CO₂R⁶, CONR⁶R⁶, C₁-C₄-alkyl, C₁-C₄-haloalkyl, C₃-C₆ cycloalkyl, and 4- to 6-membered heterocyclyl.

In some embodiments, R^{2a} is independently at each occurrence selected from ═O, halo, cyano, S(O)₂R⁶, CO₂R⁶, CONR⁶R⁶, C₁-C₂-alkyl, C₁-C₂-haloalkyl, C₃-C₄ cycloalkyl, and 4- to 6-membered heterocyclyl.

In some embodiments, R^{2a} is independently at each occurrence selected from ═O, halo, cyano, S(O)₂R⁶, CO₂R⁶, CONR⁶R⁶, C₁-C₂-alkyl, C₁-C₂-haloalkyl, cyclopropyl, cyclobutyl, and 4-membered heterocyclyl.

In some embodiments, R^{2a} is independently at each occurrence selected from ═O, halo, cyano, S(O)₂R⁶, CO₂R⁶, CONR⁶R⁶, C₁-C₂-alkyl, C₁-C₂-haloalkyl, and 4-membered heterocyclyl.

In some embodiments, R^{2a} is independently at each occurrence selected from ═O, halo, OR⁷, C₁-C₄-alkyl, and C₁-C₄-haloalkyl.

In some embodiments, R^{2a} is independently at each occurrence selected from halo, OR⁷, C₁-C₄-alkyl, and C₁-C₄-haloalkyl. In some embodiments, R^{2a} is independently at each occurrence selected from halo, C₁-C₄-alkyl, and C₁-C₄-haloalkyl. In some embodiments, R^{2a} is independently at each occurrence selected from C₁-alkyl and C₁-haloalkyl. In some embodiments, R^{2a} is C₁-C₄-alkyl, e.g., methyl.

In some embodiments, n is an integer selected from 0, 1, and 2. In some embodiments, n is 2. In some embodiments, n is 0. Preferably, n is 1. In some embodiments, where n is 1, R² is attached to X³.

In some embodiments, n1 is 0. Preferably, n1 is 1. In some embodiments, where n1 is 1, R^{2a} is attached to X³.

In some embodiments, n is an integer selected from 0, 1, and 2. In some embodiments, n2 is 2. In some embodiments, n2 is 0. In some embodiments, n2 is 1.

In some embodiments, n3 is 0. Preferably, n3 is 1. In some embodiments, where n3 is 1, R^{2a} is attached to X³.

In some embodiments, n7 is 0. In some embodiments, n7 is 1. In some embodiments, n7 is 2. In some embodiments, n7 is 3.

In some embodiments, n8 is 0. In some embodiments, n8 is 1. In some embodiments, n8 is 2. In some embodiments, n8 is 3.

In some embodiments, R¹¹ is

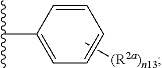

wherein n13 is independently an integer selected from 0, 1, 2, 3, 4, and 5.

In some embodiments, R¹¹ is

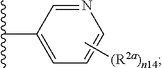

wherein n14 is independently an integer selected from 0, 1, 2, 3, and 4.

In some embodiments, R¹¹ is

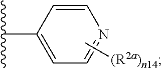

wherein n14 is independently an integer selected from 0, 1, 2, 3, and 4.

In some embodiments, R¹¹ is

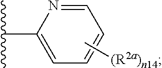

wherein n14 is independently an integer selected from 0, 1, 2, 3, and 4.

In some embodiments, R¹¹ is

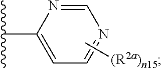

wherein n15 is independently an integer selected from 0, 1, 2, and 3.

In some embodiments, R¹¹ is

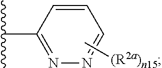

wherein n15 is independently an integer selected from 0, 1, 2, and 3.

In some embodiments, R¹¹ is

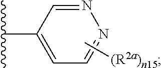

wherein n15 is independently an integer selected from 0, 1, 2, and 3.

In some embodiments, R¹¹ is

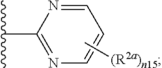

wherein n15 is independently an integer selected from 0, 1, 2, and 3.

In some embodiments, R¹¹

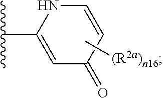

is wherein n16 is independently an integer selected from 0, 1, 2, 3, and 4.

In some embodiments, R^{2a} is independently at each occurrence selected from ═O, halo, nitro, cyano, NR⁵R⁶, OR⁷, SR⁶, S(O)₂R⁶, SO₂NR⁶R⁶, CO₂R^{S}, C(O)R⁶, CONR⁶R⁶, C₁-C₄-alkyl, C₁-C₄-haloalkyl, C₃-C₆cycloalkyl, and 4- to 6-membered heterocyclyl.

In some embodiments, R^{2a} is independently at each occurrence selected from ═O, halo, cyano, CO₂R⁶, C(O)R⁶, C₁-C₄-alkyl, C₁-C₄-haloalkyl, C₃-C₆ cycloalkyl, and 4- to 6-membered heterocyclyl.

In some embodiments, R^{2a} is independently at each occurrence selected from ═O, halo, cyano, S(O)₂R⁶, CO₂R⁶, CONR⁶R⁶, C₁-C₄-alkyl, C₁-C₄-haloalkyl, C₃-C₆ cycloalkyl, and 4- to 6-membered heterocyclyl.

In some embodiments, R^{2a} is independently at each occurrence selected from ═O, halo, cyano, S(O)₂R⁶, CO₂R⁶, CONR⁶R⁶, C₁-C₂-alkyl, C₁-C₂-haloalkyl, C₃-C₄ cycloalkyl, and 4- to 6-membered heterocyclyl.

In some embodiments, R^{2a} is independently at each occurrence selected from ═O, halo, cyano, S(O)₂R⁶, CO₂R⁶, CONR⁶R⁶, C₁-C₂-alkyl, C₁-C₂-haloalkyl, cyclopropyl, cyclobutyl, and 4-membered heterocyclyl.

In some embodiments, R^{2a} is independently at each occurrence selected from ═O, halo, cyano, S(O)₂R³, CO₂R⁶, CONR⁶R⁶, C₁-C₂-alkyl, C₁-C₂-haloalkyl, and 4-membered heterocyclyl.

In some embodiments, R^{2a} is independently at each occurrence selected from ═O, halo, OR⁷, C₁-C₄-alkyl, and C₁-C₄-haloalkyl.

In some embodiments, R^{2a} is independently at each occurrence selected from halo, OR⁶, C₁-C₄-alkyl, and C₁-C₄-haloalkyl. In some embodiments, R^{2a} is independently at each occurrence selected from halo, C₁-C₄-alkyl, and C₁-C₄-haloalkyl. In some embodiments, R^{2a} is independently at each occurrence selected from C₁-alkyl and C₁-haloalkyl. In some embodiments, R^{2a} is C₁-C₄-alkyl, e.g., methyl.

In some embodiments, n13 is 0. In some embodiments, n13 is 1. In some embodiments, n13 is 2. In some embodiments, n13 is 3.

In some embodiments, n14 is 0. In some embodiments, n14 is 1. In some embodiments, n14 is 2. In some embodiments, n14 is 3.

In some embodiments, n15 is 0. In some embodiments, n15 is 1. In some embodiments, n15 is 2. In some embodiments, n15 is 3.

In some embodiments, n16 is 0. In some embodiments, n16 is 1. In some embodiments, n16 is 2.

In embodiment B, the present disclosure provides a compound of formula (IA), a tautomer, a stereoisomer or a mixture of stereoisomers, a pharmaceutically acceptable salt, a hydrate, a deuterated derivative, or an N-oxide thereof:

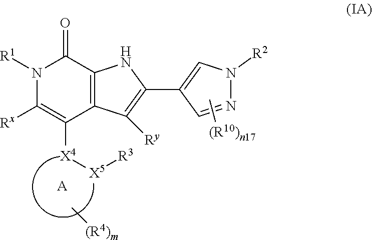

wherein:
Ring A is independently selected from phenyl, 5-membered heterocyclyl and 6-membered heterocyclyl, wherein X⁴ is independently selected from carbon and nitrogen and X⁵ is independently selected from carbon and nitrogen;
R¹ is independently selected from C₁-C₃-alkyl, C₁-C₃-fluoroalkyl, C₃-C₄-cycloalkyl and 4-membered heterocycloalkyl;
R² is independently selected from C₁-C₄-haloalkyl, ethyl, cyano, nitro, isopropyl, tert-butyl, cyclopropyl, and SF₅;
R³ is independently selected from R^{3a}, OR^{3b}, and NR⁶R^{3b};
R^{3a} is independently selected from H, CN, C₁-C₄-alkyl, C₂-C₄-alkenyl, C₂-C₄-alkynyl, C₁-C₄-haloalkyl, C₂-C₄-haloalkenyl, and C₀-C₃-alkylene-R^{3c}; wherein R^{3c} is independently at each occurrence selected from C₃-C₈-cycloalkyl, C₅-C₈-cycloalkenyl, 5- to 8-membered heterocycloalkenyl, 3- to 8-membered heterocycloalkyl, phenyl and 5- or 6-membered heteroaryl; wherein where R^{3c} is cycloalkyl, heterocycloalkyl, cycloalkenyl, or heterocycloalkenyl, R^{3c} is optionally substituted with from 1 to 4 R⁸ groups and where R^{3c} is phenyl or heteroaryl, R^{3c} is optionally substituted with from 1 to 5 R⁹ groups;
R^{3b} is independently selected from C₁-C₄-alkyl, C₂-C₄-alkylene-O—C₁-C₄-alkyl, C₁-C₄-haloalkyl and C₀-C₃-alkylene-R^{3d}; wherein R^{3d} is independently at each occurrence selected from C₃-C₈-cycloalkyl, 3- to 8-membered heterocycloalkyl, phenyl and 5- or 6-membered heteroaryl; wherein where R^{3d} is cycloalkyl or heterocycloalkyl, R^{3d} is optionally substituted with from 1 to 4 R⁸ groups and where R^{3d} is phenyl or heteroaryl, R^{3d} is optionally substituted with from 1 to 5 R⁹ groups;
R⁴ is independently at each occurrence selected from ═O, ═S, halo, nitro, cyano, C₀-C₄-alkylene-NR⁵R⁶, C₀-C₄-alkylene-OR⁷, SR⁶, SOR⁶, C₀-C₄-alkylene-S(O)₂R⁶, SO₂NR⁶R⁶, C₀-C₄-alkylene-CO₂R⁶, C₀-C₄-alkylene-C(O)R⁶, C₀-C₄-alkylene-CONR⁶R⁶, C₁-C₄-alkyl, C₂-C₄-alkenyl, C₂-C₄-alkynyl, C₁-C₄-haloalkyl, C₃-C₆-cycloalkyl, and C₀-C₄-alkylene-R^{4c};
R^{4c} is independently at each occurrence selected from C₃-C₆-cycloalkyl and 4- to 6-membered heterocycloalkyl;
R⁵ is independently at each occurrence selected from H, C₁-C₄-alkyl, C(O)—C₁-C₄-alkyl and S(O)₂—C₁-C₄-alkyl; or R⁵ and R⁶, together with the nitrogen atom to which they are attached form a C₅-C₈-heterocycloalkyl group optionally substituted with from 1 to 4 R⁸ groups;
R⁶ is independently at each occurrence selected from H and C₁-C₄-alkyl; or where two R⁶ groups are attached to the same nitrogen, those two R⁶ groups together with the nitrogen atom to which they are attached optionally form a C₅-C₈-heterocycloalkyl group optionally substituted with from 1 to 4 R⁸ groups;
R⁷ is independently at each occurrence selected from H, C₁-C₄-alkyl, C(O)—C₁-C₄-alkyl and C₁-C₄-haloalkyl;
R⁸ is independently at each occurrence selected from ═O, ═S, fluoro, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl and cyclopropyl;
R⁹ is independently at each occurrence selected from halo, nitro, cyano, C₀-C₄-alkylene-NR⁵R⁶, C₀-C₄-alkylene-OR^{7a}, C₀-C₄-alkylene-SR⁶, C₀-C₄-alkylene-SOR⁶, C₀-C₄-alkylene-S(O)₂R⁶, C₀-C₄-alkylene-SO₂NR⁶R⁶, C₀-C₄-alkylene-C₂R⁶, C₀-C₄-alkylene-C(O)R⁶, C₀-C₄-alkylene-CONR⁶R⁶, C₀-C₄-alkylene-R^{9a}, C₁-C₄-alkyl, C₂-C₄-alkenyl, C₂-C₄-alkynyl and C₁-C₄-haloalkyl; or where two R⁹ groups are attached to adjacent atoms, those two R⁹ groups together with the atoms to which they are attached optionally form a 5- or 6-membered heterocycloalkyl group optionally substituted with from 1 to 4 R⁸ groups;
R^{7a} is independently at each occurrence selected from H, C₁-C₄-alkyl, C(O)—C₁-C₄-alkyl, C₀-C₄-alkylene-NR₅R₆, —C₀-C₄-alkyl-O—R⁷, C₀-C₄-alkylene-SRS, Co—C₄-alkylene-SOR⁶, C₀-C₄-alkylene-S(O)₂R⁶, C₀-C₄-alkylene-SO₂NR⁶R⁶, C₀-C₄-alkylene-CO₂R⁶, C₀-C₄-alkylene-C(O)R⁶, C₀-C₄-alkylene-CONR⁶R⁶ and C₁-C₄-haloalkyl;
R^{9a} is independently at each occurrence selected from C₃-C₆-cycloalkyl and 4- to 6-membered heterocycloalkyl;
R¹⁰ is independently at each occurrence selected from halo, C₁-C₄-alkyl, C₁-C₄-haloalkyl, nitro, cyano, NR⁵R⁶, OR⁷, SRS, SORS, S(O)₂R⁹, SO₂NRR, CO₂R⁶, C(O)R⁶, CONR⁶R⁶, C₁-C₄-alkyl, C₂-C₄-alkenyl, C₂-C₄-alkynyl and 4-membered heterocycloalkyl;
Rₓ and R_{y} are each independently selected from H, halo, nitro, cyano, NR⁵R⁶, OR⁷, SR⁶, SOR⁶, S(O)₂R⁶, SO₂NR⁶R⁶, CO₂R, C(O)R, CONR⁶R⁶, C₁-C₄-alkyl, C₂-C₄-alkenyl, C₂-C₄-alkynyl, C₁-C₄-haloalkyl, C₃-C₄-cycloalkyl and 4-membered heterocycloalkyl;
m is an integer selected from 0, 1, 2, 3 and 4;
n¹⁷ is an integer selected from 0, 1 and 2;
wherein any of the aforementioned alkyl, alkylene, alkenyl, cycloalkyl or heterocycloalkyl groups is optionally substituted, where chemically possible, by 1 to 5 substituents which are each independently at each occurrence selected from the group consisting of: C₁-C₄-alkyl, oxo, fluoro, nitro, cyano, NR^{a}R^{b}, OR^{a}, SR^{a}, CO₂R^{a}, C(O)R^{a}, CONR^{a}R^{a}, S(O)R^{a} and S(O)₂R^{a}; wherein R^{a} is independently at each occurrence selected from H, C₁-C₄-alkyl and C₁-C₄-haloalkyl; and R^{b} is independently at each occurrence selected from H, C₁-C₄-alkyl, C(O)—C₁-C₄-alkyl and S(O)₂—C₁-C₄-alkyl.

In embodiment C, the present disclosure provides a compound of formula (IA), a tautomer, a stereoisomer or a mixture of stereoisomers, a pharmaceutically acceptable salt, a hydrate, a deuterated derivative, or an N-oxide thereof:

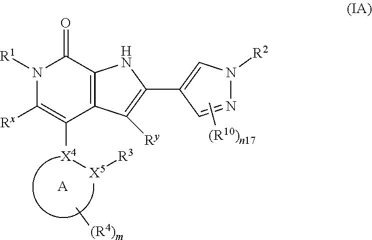

wherein:
Ring A is independently selected from phenyl, 5-membered heterocyclyl and 6-membered heterocyclyl, wherein X⁴ is independently selected from carbon and nitrogen and X⁵ is independently selected from carbon and nitrogen;
R¹ is independently selected from C₁-C₃-alkyl, C₁-C₃-fluoroalkyl, C₃-C₄-cycloalkyl and 4-membered heterocycloalkyl;
R² is C₁-C₄-haloalkyl,
R³ is independently selected from R^{3a}, OR^{3b}, and NR⁶R^{3b};
R^{3a} is independently selected from H, CN, C₁-C₄-alkyl, C₂-C₄-alkenyl, C₂-C₄-alkynyl, C₁-C₄-haloalkyl, C₂-C₄-haloalkenyl, and C₀-C₃-alkylene-R^{3c}; wherein R^{3c} is independently at each occurrence selected from C₃-C₈-cycloalkyl, C₅-C₈-cycloalkenyl, 5- to 8-membered heterocycloalkenyl, 3- to 8-membered heterocycloalkyl, phenyl and 5- or 6-membered heteroaryl; wherein where R^{3c} is cycloalkyl, heterocycloalkyl, cycloalkenyl, or heterocycloalkenyl, R^{3c} is optionally substituted with from 1 to 4 R⁸ groups and where R^{3c} is phenyl or heteroaryl, R^{3c} is optionally substituted with from 1 to 5 R⁹ groups;
R^{3b} is independently selected from C₁-C₄-alkyl, C₂-C₄-alkylene-O—C₁-C₄-alkyl, C₁-C₄-haloalkyl and C₀-C₃-alkylene-R^{3d}; wherein R^{3d} is independently at each occurrence selected from C₃-C₈-cycloalkyl, 3- to 8-membered heterocycloalkyl, phenyl and 5- or 6-membered heteroaryl; wherein where R^{3d} is cycloalkyl or heterocycloalkyl, R^{3d} is optionally substituted with from 1 to 4 R⁸ groups and where R^{3d} is phenyl or heteroaryl, R^{3d} is optionally substituted with from 1 to 5 R⁹ groups;
R⁴ is independently at each occurrence selected from ═O, ═S, halo, nitro, cyano, C₀-C₄-alkylene-NR⁵R⁶, C₀-C₄-alkylene-OR⁷, SR⁶, SOR⁶, C₀-C₄-alkylene-S(O)₂R⁶, SO₂NR⁶R⁶, C₀-C₄-alkylene-CO₂R⁶, C₀-C₄-alkylene-C(O)R⁶, C₀-C₄-alkylene-CONR⁶R⁶, C₁-C₄-alkyl, C₂-C₄-alkenyl, C₂-C₄-alkynyl, C₁-C₄-haloalkyl, C₃-C₆-cycloalkyl, and C₀-C₄-alkylene-R^{4c};
R^{4c} is independently at each occurrence selected from C₃-C₆-cycloalkyl and 4- to 6-membered heterocycloalkyl;
R⁵ is independently at each occurrence selected from H, C₁-C₄-alkyl, C(O)—C₁-C₄-alkyl and S(O)₂—C₁-C₄-alkyl; or R⁵ and R⁶, together with the nitrogen atom to which they are attached form a C₅-C₈-heterocycloalkyl group optionally substituted with from 1 to 4 R⁸ groups;
R⁶ is independently at each occurrence selected from H and C₁-C₄-alkyl; or where two R⁶ groups are attached to the same nitrogen, those two R⁶ groups together with the nitrogen atom to which they are attached optionally form a C₅-C₈-heterocycloalkyl group optionally substituted with from 1 to 4 R⁸ groups;
R⁷ is independently at each occurrence selected from H, C₁-C₄-alkyl, C(O)—C₁-C₄-alkyl and C₁-C₄-haloalkyl;
R⁸ is independently at each occurrence selected from ═O, ═S, fluoro, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl and cyclopropyl;
R⁹ is independently at each occurrence selected from halo, nitro, cyano, C₀-C₄-alkylene-NR⁶R⁶, C₀-C₄-alkylene-OR^{7a}, C₀-C₄-alkylene-SR⁶, C₀-C₄-alkylene-SOR⁶, C₀-C₄-alkylene-S(O)₂R⁶, C₀-C₄-alkylene-SO₂NR⁶R⁶, C₀-C₄-alkylene-C₂R⁶, C₀-C₄-alkylene-C(O)R⁶, C₀-C₄-alkylene-CONR⁶R⁶, C₀-C₄-alkylene-R^{9a}, C₁-C₄-alkyl, C₂-C₄-alkenyl, C₂-C₄-alkynyl, C₁-C₄-haloalkyl; or where two R⁹ groups are attached to adjacent atoms, those two R⁹ groups together with the atoms to which they are attached optionally form a 5- or 6-membered heterocycloalkyl group optionally substituted with from 1 to 4 R^{o} groups:
R^{7a} is independently at each occurrence selected from H, C₁-C₄-alkyl, C(O)—C₁-C₄-alkyl, C₀-C₄-alkylene-NR⁵R⁶, —C₀-C₄-alkyl-O—R⁷, C₀-C₄-alkylene-SRS, C₀-C₄-alkylene-SOR⁶, C₀-C₄-alkylene-S(O)₂R⁶, C₀-C₄-alkylene-SO₂NR⁶R⁶, C₀-C₄-alkylene-CO₂R⁶, C₀-C₄-alkylene-C(O)R⁶, C₀-C₄-alkylene-CONR⁶R⁶ and C₁-C₄-haloalkyl;
R^{9a} is independently at each occurrence selected from C₃-C₆-cycloalkyl and 4- to 6-membered heterocycloalkyl;
R¹⁰ is independently at each occurrence selected from halo, C₁-C₄-alkyl, C₁-C₄-haloalkyl, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₂-C₄-alkenyl, C₂-C₄-alkynyl and 4-membered heterocycloalkyl;
R^{x} and R^{y} are each independently selected from H, halo, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl, C₃-C₄-cycloalkyl and 4-membered heterocycloalkyl;
m is an integer selected from 0, 1, 2, 3 and 4;
n¹⁷ is an integer selected from 0, 1 and 2;
wherein any of the aforementioned alkyl, alkylene, alkenyl, cycloalkyl or heterocycloalkyl groups is optionally substituted, where chemically possible, by 1 to 5 substituents which are each independently at each occurrence selected from the group consisting of: C₁-C₄-alkyl, oxo, fluoro, nitro, cyano, NR^{a}R^{b}, OR^{a}, SR^{a}, CO₂R^{a}, C(O)R^{a}, CONR^{a}R^{a}, S(O)R^{a} and S(O)₂R^{a}; wherein R^{a} is independently at each occurrence selected from H, C₁-C₄-alkyl and C₁-C₄-haloalkyl; and R^{b} is independently at each occurrence selected from H, C₁-C₄-alkyl, C(O)—C₁-C₄-alkyl and S(O)₂—C₁-C₄-alkyl.

In one or more embodiments, a compound of formula (IA) may be an enantiomer, a mixture of enantiomers, a racemate, a diastereoisomer, a mixture of diastereoisomers, a geometric isomer, a mixture of geometric isomers, a tautomer or a mixture of tautomers. A compound of formula (IA) may also be in the form of a solvate or hydrate.

In one or more embodiments, the compound of formula (IA) is a compound of formula (XII):

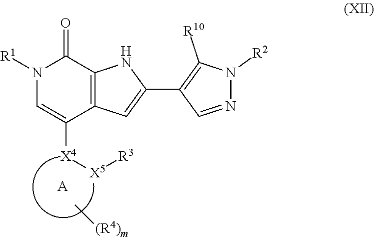

wherein X⁴, X⁵, Ring A, R¹, R², R³, R⁴, R¹⁰ and m are as described for compounds of formula (IA) in embodiment B or C above.

In one or more embodiments, the compound of formula (IA) is a compound of formula (XIIIa) or (XIIIb):

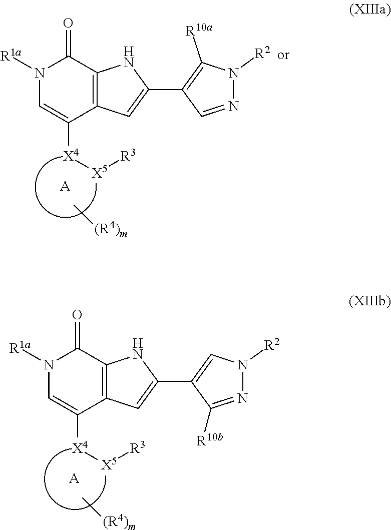

wherein X⁴, X⁵, Ring A, R², R³, R⁴ and m are as described for compounds of formula (IA) in embodiment B or C above; and
wherein:
R^{1a} is selected from methyl and ethyl;
R^{10a} is selected from H and methyl; and
R^{10b} is selected from H and methyl.

In one or more embodiments, the compound of formula (IA) is a compound of formula (XIV):

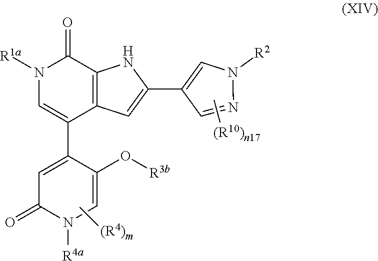

wherein R², R^{3b}, R⁴, R¹⁰ and n¹⁷ are as described for compounds of formula (IA) in embodiment B or C above; and wherein.
R^{1a} is selected from methyl and ethyl;
m is an integer selected from 0, 1, or 2; and
R^{4a} is independently selected from H, C₁-C₄-alkyl, cyclopropyl, cyclobutyl, and C₀-C₄-alkylene-R^{4c}. In some embodiments, R^{4c} is independently at each occurrence selected from C₃-C₆-cycloalkyl and 4- to 6-membered heterocycloalkyl. In some embodiments, R^{4a} is independently selected from H, C1-C4-alkyl, and cyclopropyl. In some embodiments, R^{4a} is independently selected from H, methyl, cyclopropyl, and oxetan-3-yl. R^{4a} may be selected from H, methyl, and cyclopropyl.

In some embodiments, the compound of formula (IA) is a compound of formula (XV):

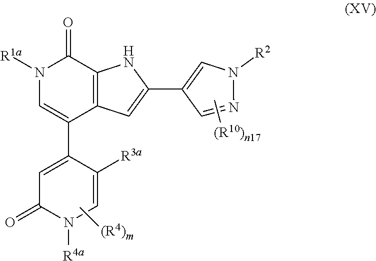

wherein R², R^{3a}, R⁴, R¹⁰ and n¹⁷ are as described for compounds of formula (IA) in embodiment B or C above; and wherein:
R^{1a} is selected from methyl and ethyl;
m is an integer selected from 0, 1, or 2; and
R^{4a} is independently selected from H, C₁-C₄-alkyl, cyclopropyl, cyclobutyl, and C₀-C₄-alkylene-R^{4c}. R^{4c} is independently at each occurrence selected from C₃-C₅-cycloalkyl and 4- to 5-membered heterocycloalkyl. R^{4c} may be independently selected from a C₃-C₅-cycloalkyl. R^{4a} may be independently selected from H, C₁-C₄-alkyl, and cyclopropyl.

In some embodiments, the compound of formula (IA) is a compound of formula (XVI):

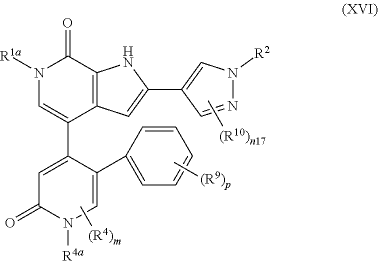

wherein R², R⁴, R⁹, R¹⁰ and n¹⁷ are as described for compounds of formula (IA) in embodiment B or C above; and wherein:
R^{1a} is selected from methyl and ethyl;
m is an integer selected from 0, 1, or 2;
p is an integer selected from 0, 1, 2, 3, 4 and 5; and
R^{4a} is independently selected from H, C₁-C₄-alkyl, cyclopropyl, cyclobutyl, and C₀-C₄-alkylene-R^{4c}. R^{4c} is independently at each occurrence selected from C₃-C₅-cycloalkyl and 4- to 5-membered heterocycloalkyl. R^{4c} may be independently selected from a C₃-C₅-cycloalkyl. R^{4a} may be independently selected from H, C₁-C₄-alkyl, and cyclopropyl.

In some embodiments, the compound of formula (IA) is a compound of formula (XVIIa) or (XVIIb):

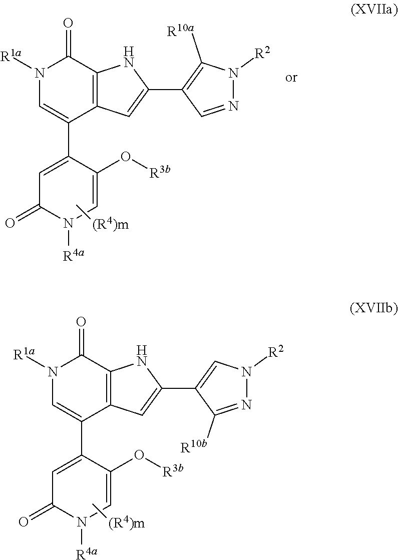

wherein R², R^{3b} and R⁴ are as described for compounds of formula (IA) in embodiment B or C above; and wherein:
R^{1a} is selected from methyl and ethyl;
m is an integer selected from 0, 1, or 2; and
R^{4a} is independently selected from H, C₁-C₄-alkyl, cyclopropyl, cyclobutyl, and C₀-C₄-alkylene-R^{4c}. R^{4c} is independently at each occurrence selected from C₃-C₅-cycloalkyl and 4- to 5-membered heterocycloalkyl. R^{4C} may be independently selected from a C₃-C₅-cycloalkyl. R^{4a} may be independently selected from H, C₁-C₄-alkyl, and cyclopropyl;
R^{10a} is selected from H and methyl;
R^{10b} is selected from H and methyl.

In some embodiments, the compound of formula (IA) is a compound of formula (XVIII):

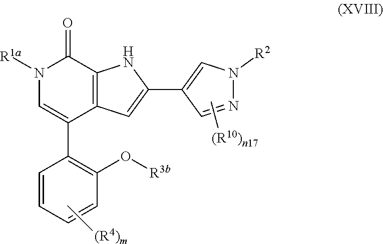

wherein R², R³, R⁴, R¹⁰, n¹⁷ and m are as described for compounds of formula (IA) in embodiment B or C above;
R^{1a} is selected from methyl and ethyl.

In some embodiments, the compound of formula (IA) is a compound of formula (XIX):

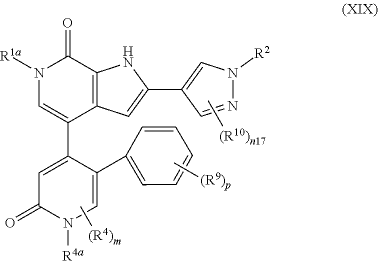

wherein R², R⁴, R⁹, R¹⁰, and n¹⁷ are as described for compounds of formula (IA) in embodiment B or C above;
m is an integer selected from 0, 1, or 2;
p is an integer selected from 0, 1, 2, 3, 4, and 5;
R^{1a} is selected from methyl and ethyl;
and wherein R^{4a} is independently selected from H, C₁-C₄-alkyl, cyclopropyl, cyclobutyl, and C₀-C₄-alkylene-R^{4c}. R^{4c} is independently at each occurrence selected from C₃-C₅-cycloalkyl and 4- to 5-membered heterocycloalkyl. R^{4c} may be independently selected from a C₃-C₅-cycloalkyl.

In some embodiments, the compound of formula (IA) is a compound of formula (XX):

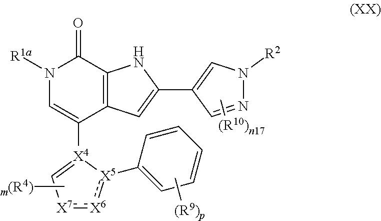

Wherein X⁴, X⁵, R², R⁴, R⁹, R¹⁰ and n¹⁷ are as described for compounds of formula (IA) in embodiment B or C above; and wherein:
each [symbol] is independently selected from a single bond and a double bond;
R^{1a} is selected from methyl and ethyl;
X⁶ is independently selected from carbon and nitrogen;
X⁷ is independently selected from carbon and nitrogen;
m is an integer selected from 0, 1, 2, or 3; and
p is an integer selected from 0, 1, 2, 3, 4 and 5.

In some embodiments, R^{1a} may be selected from methyl and ethyl. R^{1a} may be methyl. R^{1a} may be ethyl.

In some embodiments, R² may be selected from C₁-C₄-haloalkyl, ethyl, isopropyl, tert-butyl and cyclopropyl. R² may be C₁-C₄-haloalkyl e.g., C₁-C₄-fluoroalkyl. R² may be selected from —CF₃, —CHF₂, —CH₂—CF₃, and —CH₂—CH₂F.

In some embodiments, R¹⁰ may be selected from halo, C₁-C₄-alkyl, C₁-C₄-haloalkyl, C₂-C₄-alkenyl, C₂-C₄-alkynyl and 4-membered heterocycloalkyl. R¹⁰ may be C₁-C₄-alkyl, e.g., methyl.

In some embodiments, n₁₇ is an integer selected from 0 and 1. n¹⁷ may be 0. n₁₇ may be 1.

In some embodiments, R¹ is C₁-C₃-alkyl. In some embodiments, R¹ is C₁-C₃-fluoroalkyl. In some embodiments, R¹ is C₃-cycloalkyl. In some embodiments, R¹ is independently selected from C₁-alkyl, C₁-fluoroalkyl, C₂-alkyl, C₂-fluoroalkyl and C₃-cycloalkyl. Preferably, R¹ is C₁-alkyl, i.e., methyl.

In some embodiments, X⁴ is carbon. In some embodiments, X⁴ is nitrogen.

In some embodiments, X⁵ is carbon. In some embodiments, X⁵ is nitrogen.

In some embodiments, Ring A is a phenyl ring. In some embodiments, Ring A is a 5- or 6-membered heterocyclyl. In some embodiments, Ring A is a 5- or 6-membered heteroaryl. In some embodiments, Ring A is a 5-membered heteroaryl ring. In some embodiments, Ring A is a 6-membered heterocyclyl ring. In some embodiments, Ring A is a 6-membered heteroaryl ring.

In some embodiments, when Ring A is a 5-membered heterocyclyl it is not a pyrrolidone.

In some embodiments, Ring A is phenyl. In some embodiments, Ring A is pyridone. Said pyridone may be substituted on the nitrogen with a C₁-C₄-alkyl group, a cyclopropyl, a cyclobutyl, or a 4-membered heterocycloalkyl group. Said pyridone may be substituted on the nitrogen with either a C₁-C₄-alkyl group or a cyclopropyl group. In some embodiments, Ring A is N—C₁-C₄-alkyl pyridone. In some embodiments, Ring A is pyridine. In some embodiments, Ring A is pyrrole. In some embodiments, Ring A is imidazole. In some embodiments, Ring A is pyrazole. In some embodiments, Ring A is triazole. In some embodiments, Ring A is tetrazole.

In some embodiments,

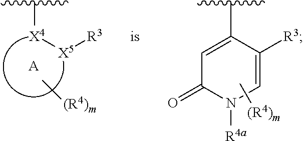

wherein R^{4a} is selected from H, C₁-C₄-alkyl (e.g., methyl), cyclopropyl, cyclobutyl, C₀-C₄-alkylene-R^{4a}, SOR⁶, S(O)₂R⁶, SO₂NR⁶R⁶, C₀-C₄-alkylene-CO₂R⁶, C₀-C₄-alkylene-C(O)R⁶, C₀-C₄-alkylene-CONR⁶R⁶, C₁-C₄-alkyl-S(O)₂R⁶, C₂-C₄-alkylene-NR⁵R⁶, C₂-C₄-alkylene-OR⁷, and cyclopropyl-OR^{a}; optionally wherein R³ is OR^{3b}. In an embodiment the heterocycloalkyl is oxetanyl or an azetidinyl.

In some embodiments,

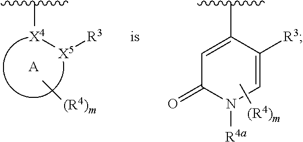

wherein R^{4a} is selected from H, C₁-C₄-alkyl (e.g., methyl), cyclopropyl, SOR⁶, S(O)₂R⁶, SO₂NR⁶R⁶, C₀-C₄-alkylene-CO₂R⁶, C₀-C₄-alkylene-C(O)R⁶, C₀-C₄-alkylene-CONR⁶R⁶, C₁-C₄-alkyl-S(O)₂R⁶, C₂-C₄-alkylene-NR⁶R⁶, C₂-C₄-alkylene-OR⁷, and cyclopropyl-OR^{a}; optionally wherein R³ is OR^{3b}.

In some embodiments,

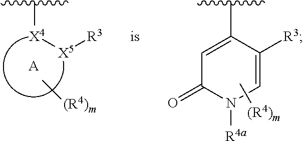

wherein R^{4a} is selected from H, C₁-C₄-alkyl (e.g., methyl), cyclopropyl, cyclobutyl, and 4-membered heterocycloalkyl; optionally wherein R³ is OR^{3b}. R^{4a} may be selected from C₁-C₄-alkyl (e.g., methyl), cyclopropyl, and oxetan-3-yl.

In some embodiments,

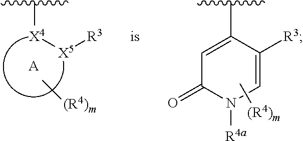

wherein R^{4a} is selected from H, C₁-C₄-alkyl (e.g., methyl), and cyclopropyl; optionally wherein R³ is OR^{3b}. R^{4a} may be selected from C₁-C₄-alkyl (e.g., methyl) and cyclopropyl.

In some embodiments,

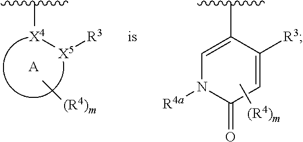

wherein R^{4a} is selected from H, C₁-C₄-alkyl (e.g., methyl), cyclopropyl, cyclobutyl, 4-membered heterocycloalkyl, SOR⁶, S(O)₂R⁶, SO₂NR⁶R⁶, C₀-C₄-alkylene-CO₂R⁶, C₀-C₄-alkylene-C(O)R⁶, C₀-C₄-alkylene-CONR⁶R⁶, C₁-C₄-alkyl-S(O)₂R⁶, C₂-C₄-alkylene-NR⁵R⁶, C₂-C₄-alkylene-OR⁷, and cyclopropyl-OR^{a}; optionally wherein R³ is OR^{3b}.

In some embodiments,

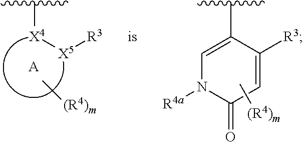

wherein R^{4a} is selected from H, C₁-C₄-alkyl (e.g., methyl), cyclopropyl, SOR⁶, S(O)₂R⁶, SO₂NR⁶R⁶, C₀-C₄-alkylene-CO₂R⁶, C₀-C₄-alkylene-C(O)R⁶, C₀-C₄-alkylene-CONR⁶R⁶, C₁-C₄-alkyl-S(O)₂R⁶, C₂-C₄-alkylene-NR⁵R⁶, C₂-C₄-alkylene-OR⁷, and cyclopropyl-OR^{a}; optionally wherein R³ is OR^{3b}.

In some embodiments,

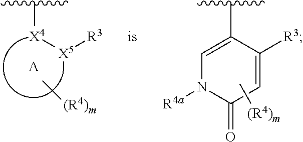

wherein R^{4a} is selected from H, C₁-C₄-alkyl (e.g., methyl), cyclopropyl, cyclobutyl, and 4-membered heterocycloalkyl; optionally wherein R³ is OR^{3b}. R^{4a} may be selected from C₁-C₄-alkyl (e.g., methyl), cyclopropyl, and oxetan-3-yl.

In some embodiments,

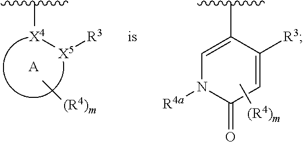

wherein R^{4a} is selected from H, C₁-C₄-alkyl (e.g., methyl), and cyclopropyl; optionally wherein R³ is OR^{3b}. R^{4a} may be selected from C₁-C₄-alkyl (e.g., methyl) and cyclopropyl.

In some embodiments,

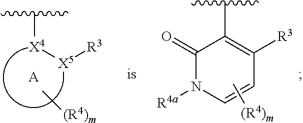

wherein R^{4a} is selected from H, C₁-C₄-alkyl (e.g., methyl), cyclopropyl, cyclobutyl, 4-membered heterocycloalkyl, SOR⁶, S(O)₂R⁶, SO₂NR⁶R⁶, C₀-C₄-alkylene-CO₂R⁶, C₀-C₄-alkylene-C(O)R⁶, C₀-C₄-alkylene-CONR⁶R⁶, C₁-C₄-alkyl-S(O)₂R⁶, C₂-C₄-alkylene-NR⁵R⁶, C₂-C₄-alkylene-OR⁷, and cyclopropyl-OR^{a}; optionally wherein R³ is OR^{3b}.

In some embodiments,

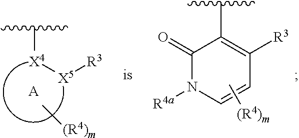

wherein R^{4a} is selected from H, C₁-C₄-alkyl (e.g., methyl), cyclopropyl, SOR⁶, S(O)₂R⁶, SO₂NR⁶R⁶, C₀-C₄-alkylene-CO₂R⁶, C₀-C₄-alkylene-C(O)R⁶, C₀-C₄-alkylene-CONR⁶R⁶, C₁-C₄-alkyl-S(O)₂R⁶, C₂-C₄-alkylene-NR⁶R⁶, C₂-C₄-alkylene-OR⁷, and cyclopropyl-OR^{a}; optionally wherein R³ is OR^{3b}.

In some embodiments,

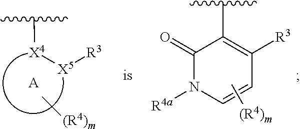

wherein R^{4a} is selected from H, C₁-C₄-alkyl (e.g., methyl), cyclopropyl, cyclobutyl, and 4-membered heterocycloalkyl; optionally wherein R³ is OR^{3b}. R^{4a} may be selected from C₁-C₄-alkyl (e.g., methyl), cyclopropyl, and oxetan-3-yl.

In some embodiments,

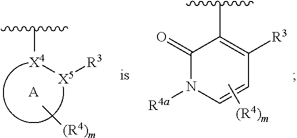

wherein R^{4a} is selected from H, C₁-C₄-alkyl (e.g., methyl), and cyclopropyl; optionally wherein R³ is OR^{3b}. R^{4a} may be selected from C₁-C₄-alkyl (e.g., methyl) and cyclopropyl.

In some embodiments,

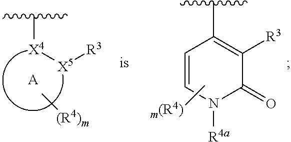

wherein R^{4a} is selected from H, C₁-C₄-alkyl (e.g., methyl), cyclopropyl, cyclobutyl, 4-membered heterocycloalkyl, SOR, S(O)₂R⁶, SO₂NR⁶R⁶, C₀-C₄-alkylene-CO₂R⁶, C₀-C₄-alkylene-C(O)R, C₀-C₄-alkylene-CONR⁶R⁶, C₁-C₄-alkyl-S(O)₂R⁶, C₂-C₄-alkylene-NR⁵R⁶, C₂-C₄-alkylene-OR⁷, and cyclopropyl-OR^{a}; optionally wherein R³ is OR^{3b}.

In some embodiments,

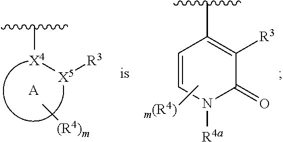

wherein R^{4a} is selected from H, C₁-C₄-alkyl (e.g., methyl), cyclopropyl, SOR⁶, S(O)₂R⁶, SO₂NR⁶R⁶, C₀-C₄-alkylene-CO₂R⁶, C₀-C₄-alkylene-C(O)R⁶. C₀-C₄-alkylene-CONR⁶R⁶, C₁-C₄-alkyl-S(O)₂R⁶, C₂-C₄-alkylene-NR⁶R⁶, C₂-C₄-alkylene-OR⁷, and cyclopropyl-OR^{a}; optionally wherein R³ is OR^{3b}.

In some embodiments,

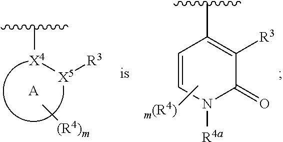

wherein R^{4a} is selected from H, C₁-C₄-alkyl (e.g., methyl), cyclopropyl, cyclobutyl, and 4-membered heterocycloalkyl; optionally wherein R³ is OR^{3b}. R^{4a} may be selected from C₁-C₄-alkyl (e.g., methyl) cyclopropyl, and oxetan-3-yl.

In some embodiments,

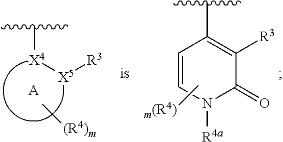

wherein R^{4a} is selected from H, C₁-C₄-alkyl (e.g., methyl), and cyclopropyl; optionally wherein R³ is OR^{3b}. R^{4a} may be selected from C₁-C₄-alkyl (e.g., methyl) and cyclopropyl.

In some embodiments,

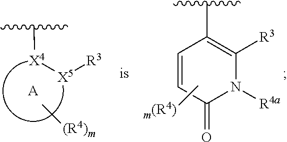

wherein R^{4a} is selected from H, C₁-C₄-alkyl (e.g., methyl), cyclopropyl, cyclobutyl, 4-membered heterocycloalkyl, SOR⁶, S(O)₂R⁶, SO₂NR⁶R⁶, C₀-C₄-alkylene-CO₂R⁶, C₀-C₄-alkylene-C(O)R⁶, C₀-C₄-alkylene-CONR⁶R⁶, C₁-C₄-alkyl-S(O)₂R⁶, C₂-C₄-alkylene-NR⁶R⁶, C₂-C₄-alkylene-OR⁷, and cyclopropyl-OR^{a}; optionally wherein R³ is OR^{3b}.

In some embodiments,

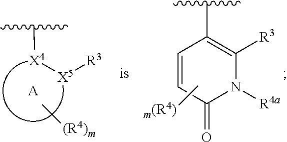

wherein R^{4a} is selected from H, C₁-C₄-alkyl (e.g., methyl), cyclopropyl, SOR⁶, S(O)₂R⁶, SO₂NR⁶R⁶, C₀-C₄-alkylene-C₂R⁶, C₀-C₄-alkylene-C(O)R⁶, C₀-C₄-alkylene-CONR⁶R⁶, C₁-C₄-alkyl-S(O)₂R⁶, C₂-C₄-alkylene-NR⁵R⁶, C₂-C₄-alkylene-OR⁷, and cyclopropyl-OR^{a}; optionally wherein R³ is OR^{3b}.

In some embodiments,

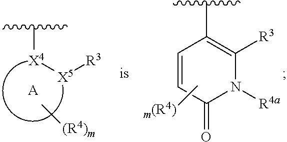

wherein R^{4a} is selected from H, C₁-C₄-alkyl (e.g., methyl), cyclopropyl, cyclobutyl, and 4-membered heterocycloalkyl; optionally wherein R³ is OR^{3b}. R^{4a} may be selected from C₁-C₄-alkyl (e.g., methyl) cyclopropyl, and oxetan-3-yl.

In some embodiments,

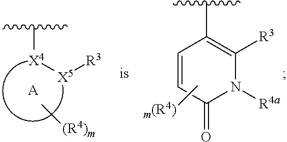

wherein R^{4a} is selected from H, C₁-C₄-alkyl (e.g., methyl), and cyclopropyl; optionally wherein R³ is OR^{3b}. R^{4a} may be selected from C₁-C₄-alkyl (e.g., methyl) and cyclopropyl.

In some embodiments,

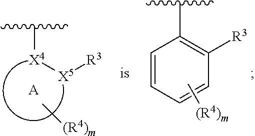

optionally wherein R³ is OR^{3b}.

In some embodiments,

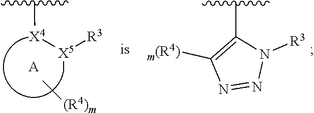

optionally wherein R³ is R^{3a}.

In some embodiments,

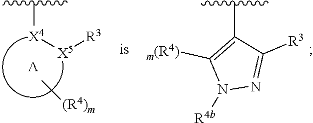

wherein R^{4b} is selected from S(O)₂R⁶, C₁-C₄-alkyl, C₁-C₄-alkyl-S(O)₂R⁶, C₁-C₄-haloalkyl, cyclopropyl, and cyclobutyl; optionally wherein R³ is R^{3a}.

In some embodiments,

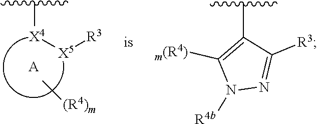

wherein R^{4b} is selected from S(O)₂R⁶, C₁-C₄-alkyl, C₁-C₄-alkyl-S(O)₂R⁶, C₁-C₄-haloalkyl, and cyclopropyl; optionally wherein R³ is R^{3a}.

In some embodiments,

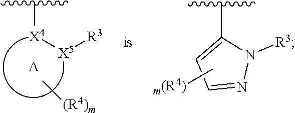

optionally wherein R³ is R^{3a}.

In some embodiments,

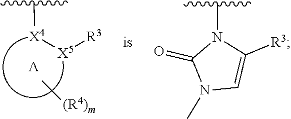

is optionally wherein R³ is R^{3a}.

In some embodiments,

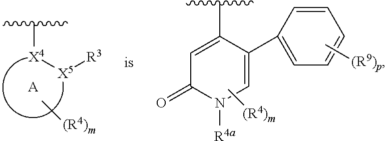

wherein
m is an integer selected from 0, 1 and 2; and
p is an integer selected from 0, 1, 2, 3, 4, and 5;
and R^{4a} is independently selected from H, C₁-C₄-alkyl, cyclopropyl, cyclobutyl, and C₀-C₄-alkylene-R^{4c}.

In some embodiments, R³ is independently selected from R^{3a} and OR^{3b}. In some embodiments, R³ is R^{3a}. In some embodiments, R³ is OR^{3b}. It may be that, where Ring A is a 5-membered heteroaryl group, R³ is R^{3a}. It may be that, where Ring A is pyridone group, R³ is R^{3a}. It may be that, where Ring A is phenyl or pyridone, R³ is OR^{3b}.

In some embodiments, R^{3a} is independently selected from H, CN, C₁-C₄-alkyl, C₂-C₄-alkenyl, C₁-C₄-haloalkyl, C₂-C₄-haloalkenyl, and C₀-C₃-alkylene-R^{3c}; wherein R^{3c} is independently at each occurrence selected from C₃-C₃-cycloalkyl, C₅-C₈-cycloalkenyl, 3- to 8-membered heterocycloalkyl, phenyl and 5- or 6-membered heteroaryl; wherein where R^{3c} is cycloalkyl or heterocycloalkyl, R^{3c} is optionally substituted with from 1 to 4 R³ groups and where R^{3c} is phenyl or heteroaryl, R^{3c} is optionally substituted with from 1 to 5 R⁹ groups;
In some embodiments, R^{3a} is independently selected from CN, C₁-C₄-alkyl, C₁-C₄-haloalkyl, C₂-C₄-haloalkenyl, and C₀-C₃-alkylene-R^{3c}; wherein R^{3c} is independently at each occurrence selected from C₃-C₈-cycloalkyl, C₅-C₈-cycloalkenyl, 5- to 8-membered heterocycloalkenyl, 3- to 8-membered heterocycloalkyl, and phenyl; wherein where R^{3c} is cycloalkyl, heterocycloalkyl, cycloalkenyl, or heterocycloalkenyl, R^{3c} is optionally substituted with from 1 to 4 R⁸ groups and where R^{3c} is phenyl, R^{3c} is optionally substituted with from 1 to 5 R⁹ groups.

In some embodiments, R^{3a} is independently selected from CN, C₁-C₄-alkyl, C₁-C₄-haloalkyl, C₂-C₄-haloalkenyl, and C₀-C₃-alkylene-R^{3c}; wherein R^{3c} is independently at each occurrence selected from C₃-C₈-cycloalkyl, C₅-C₈-cycloalkenyl, 3- to 8-membered heterocycloalkyl, and phenyl; wherein where R^{3c} is cycloalkyl or heterocycloalkyl, R^{3c} is optionally substituted with from 1 to 4 R³ groups and where R^{3c} is phenyl, R^{3c} is optionally substituted with from 1 to 5 R⁹ groups.

In some embodiments, R^{3a} is independently selected from C₃-C₄-alkyl, C₃-C₄-haloalkyl, C₃-haloalkenyl, and C₀-C₃-alkylene-R^{3c}; wherein R^{3c} is independently at each occurrence selected from C₃-C₆-cycloalkyl, C₅-C₆-cycloalkenyl, 5- to 6-membered heterocycloalkenyl, 4- to 6-membered heterocycloalkyl, and phenyl; wherein where R^{3c} is cycloalkyl, heterocycloalkyl, cycloalkenyl, or heterocycloalkenyl, R^{3c} is optionally substituted with from 1 to 4 R⁸ groups and where R^{3c} is phenyl, R^{3c} is optionally substituted with from 1 to 5 R⁹ groups.

In some embodiments, R^{3a} is independently selected from C₃-C₄-alkyl, C₃-C₄-haloalkyl, C₃-haloalkenyl, and C₀-C₃-alkylene-R^{3c}; wherein R^{3c} is independently at each occurrence selected from C₃-C₆-cycloalkyl, C₅-C₆-cycloalkenyl, 4- to 6-membered heterocycloalkyl, and phenyl; wherein where R^{3c} is cycloalkyl or heterocycloalkyl, R^{3c} is optionally substituted with from 1 to 4 R⁸ groups and where R^{3c} is phenyl, R^{3c} is optionally substituted with from 1 to 5 R⁹ groups.

In some embodiments, R^{3a} is C₀-C₃-alkylene-R^{3c}; wherein R^{3c} is independently at each occurrence selected from C₆-cycloalkyl, C₆-cycloalkenyl, 6-membered heterocycloalkenyl, 6-membered heterocycloalkyl, and phenyl; wherein where R^{3c} is cycloalkyl, cycloalkenyl, heterocycloalkenyl or heterocycloalkyl, R^{3c} is optionally substituted with from 1 to 4 R⁸ groups and where R^{3c} is phenyl, R^{3c} is optionally substituted with from 1 to 5 R⁹ groups.

In some embodiments, R^{3a} is C₀-C₃-alkylene-R^{3c}; wherein R^{3c} is independently at each occurrence selected from C₆-cycloalkyl, C₆-cycloalkenyl, 6-membered heterocycloalkyl, and phenyl; wherein where R^{3c} is cycloalkyl, cycloalkenyl or heterocycloalkyl, R^{3c} is optionally substituted with from 1 to 4 R⁸ groups and where R^{3c} is phenyl, R^{3c} is optionally substituted with from 1 to 5 R⁹ groups.

In some embodiments, R^{3a} is C₀-C₃-alkylene-R^{3c}; wherein R^{3c} is phenyl; and wherein R^{3c} is optionally substituted with from 1 to 5 R⁹ groups.

In some embodiments, R³³ is phenyl, optionally substituted with from 1 to 3 R⁹ groups. Where R^{3c}, R^{3a} or R³ are phenyl, it may be that the phenyl is substituted with from 1 to 3 R⁹ groups.

In some embodiments, R^{3a} is R^{3c}; wherein R^{3c} is phenyl; wherein R^{3c} is optionally substituted with from 1 or 2 R⁹ groups; and wherein the para-position on the phenyl group is unsubstituted.

In some embodiments, R^{3a} is C₃-C₄-alkyl. In some embodiments, R^{aa} is C₃-C₄-haloalkyl. In some embodiments, R^{3a} is C₂-C₄-haloalkenyl. In some embodiments, R^{3a} is and C₀-C₃-alkylene-R^{3c}; wherein R^{3c} is independently at each occurrence selected from C₃-C₆-cycloalkyl, C₅-C₆-cycloalkenyl, 5- to 6-membered heterocycloalkenyl, and 4- to 6-membered heterocycloalkyl. In some embodiments, R^{3a} is C₃-C₆-cycloalkyl. In some embodiments, R^{3a} is C₅-C₆-cycloalkenyl. In some embodiments, R^{3a} is 5- to 6-membered heterocycloalkenyl. In some embodiments, R^{3a} is 4-membered heterocycloalkyl. In some embodiments, R^{3a} is 5-membered heterocycloalkyl. In some embodiments, R^{3a} is 6-membered heterocycloalkyl. It may be that where R^{3a} or R^{3c} is cycloalkyl or heterocycloalkyl, R^{3c} is substituted with from 1 to 4 R³ groups.

In some embodiments, R^{3a} is C₃-C₄-alkyl. In some embodiments, R^{3a} is C₃-C₄-haloalkyl. In some embodiments, R^{3a} is C₂-C₄-haloalkenyl. In some embodiments, R^{3a} is and C₀-C₃-alkylene-R^{3c}; wherein R^{3c} is independently at each occurrence selected from C₃-C₆-cycloalkyl, C₅-C₆-cycloalkenyl, and 4- to 6-membered heterocycloalkyl. In some embodiments, R^{3a} is C₃-C₆-cycloalkyl. In some embodiments, R^{3a} C₅-C₆-cycloalkenyl. In some embodiments, R^{3a} is 4-membered heterocycloalkyl. In some embodiments, R^{3a} is 5-membered heterocycloalkyl. In some embodiments, R^{3a} is 6-membered heterocycloalkyl. It may be that where R^{3a} or R^{3c} is cycloalkyl or heterocycloalkyl, R^{3c} is substituted with from 1 to 4 R⁸ groups.

In some embodiments, R³ is selected from phenyl or —O-phenyl, wherein R³ is optionally substituted with from 1 to 5 R⁹ groups. In some embodiments, R³ is unsubstituted phenyl. In some embodiments, R³ is —O-phenyl, wherein R³ is substituted with 2 R⁹ groups.

It may be that where Ring A is 5-membered heteroaryl, R^{3a} is optionally substituted phenyl. It may be that where Ring A is 5-membered heteroaryl, R^{3a} is optionally substituted 6-membered heteroaryl.

In some embodiments, R^{3b} is independently selected from C₁-C₄-alkyl, C₂-C₄-alkylene-O—C₁-C₄-alkyl, C₁-C₄-haloalkyl and C₀-C₃-alkylene-R^{3d}; wherein R^{3d} is independently at each occurrence selected from C₃-C₈-cycloalkyl, 3- to 8-membered heterocycloalkyl, and phenyl; wherein where R^{3d} is cycloalkyl or heterocycloalkyl, R^{3d} is optionally substituted with from 1 to 4 R³ groups and where R^{3d} is phenyl, R^{3d} is optionally substituted with from 1 to 5 R⁹ groups.

In some embodiments, R^{3b} is independently selected from C₄-alkyl, C₂-C₄-alkylene-O—C₁, C₄-haloalkyl and C₀-C₃-alkylene-R^{3d}; wherein R^{3d} is independently at each occurrence selected from C₃-C₆-cycloalkyl, 4- to 6-membered heterocycloalkyl, and phenyl; wherein where R^{3d} is cycloalkyl or heterocycloalkyl, R^{3d} is optionally substituted with from 1 to 4 R³ groups and where R^{3d} is phenyl, R^{3d} is optionally substituted with from 1 to 5 R⁹ groups.

In some embodiments, R^{3b} is C₀-C₃-alkylene-R^{3d}; wherein R^{3d} is independently at each occurrence selected from C₃-C₆-cycloalkyl, 4- to 6-membered heterocycloalkyl, and phenyl; wherein where R^{3d} is cycloalkyl or heterocycloalkyl, R^{3d} is optionally substituted with from 1 to 4 R³ groups and where R^{3d} is phenyl, R^{3d} is optionally substituted with from 1 to 5 R⁹ groups.

In some embodiments, R^{3b} is C₀-C₃-alkylene-R^{3d}; wherein R^{3d} is independently at each occurrence selected from C-cycloalkyl, 6-membered heterocycloalkyl, and phenyl; wherein where R^{3d} is cycloalkyl or heterocycloalkyl, R^{3d} is optionally substituted with from 1 to 4 R³ groups and where R^{3d} is phenyl, R^{3d} is optionally substituted with from 1 to 5 R⁹ groups.

In some embodiments, R^{3b} is C₀-C₃-alkylene-R^{3d}; wherein R^{3d} is phenyl; and wherein R^{3d} is optionally substituted with from 1 to 5 R⁹ groups.

In some embodiments, R^{3b} is phenyl; optionally substituted with from 1 to 3 R⁹ groups.

In some embodiments, R^{3b} is C₀-C₃-alkylene-R^{3d}; wherein R^{3d} is independently at each occurrence selected from C₃-C₆-cycloalkyl, and 4- to 6-membered heterocycloalkyl; wherein R^{3d} is optionally substituted with from 1 to 4 R³ groups.

In some embodiments, R^{3b} is C₀-C₃-alkylene-R^{3d}; wherein R^{3d} is C₃-C₆-cycloalkyl; wherein R^{3d} is optionally substituted with from 1 to 4 R³ groups.

In some embodiments, R^{3b} is C₀-C₃-alkylene-R^{3d}; wherein R^{3d} is 4- to 6-membered heterocycloalkyl; wherein R^{3d} is optionally substituted with from 1 to 4 R⁸ groups.

In some embodiments, R^{3b} is 4- to 6-membered heterocycloalkyl; optionally substituted with from 1 to 4 R⁸ groups. In some embodiments, R^{3b} is 4-membered heterocycloalkyl; optionally substituted with from 1 to 2 R⁸ groups. In some embodiments, R^{3b} is 5-membered heterocycloalkyl; optionally substituted with from 1 to 3 R⁸ groups. In some embodiments, R^{3b} is 6-membered heterocycloalkyl; optionally substituted with from 1 to 4 R⁸ groups.

In some embodiments, R^{3b} is C₁-C₄-alkyl. In some embodiments, R^{3b} is C₂-C₄-alkylene-O—C₁-C₄-alkyl. In some embodiments, R^{3b} is C₁-C₄-haloalkyl.

In some embodiments, R^{3b} is C₃-C₄-alkyl. In some embodiments, R^{3b} is C₂-C₄-alkylene-O—C₁-C₄-alkyl. In some embodiments, R^{3b} is C₃-C₄-haloalkyl.

It may be that where Ring A is 5-membered heteroaryl, R^{3b} is optionally substituted C₆-cycloalkyl. It may be that where Ring A is 5-membered heteroaryl, R^{3b} is optionally substituted 6-membered heterocycloalkyl. It may be that where Ring A is 5-membered heteroaryl, R^{3b} is substituted or unsubstituted phenyl. It may be that where Ring A is 5-membered heteroaryl, R^{3b} is optionally substituted 6-membered heteroaryl.

In some embodiments, R⁴ is independently at each occurrence selected from cyano, C₀-C₄-alkylene-NR⁵R⁶, C₀-C₄-alkylene-OR⁷, S(O)₂R⁶, C₁-C₄-alkyl, C₀-C₄-alkylene-R^{4c}, C₀-C₄-alkylene-S(O)₂R⁶, and C₁-C₄-haloalkyl. In some embodiments, R^{4c} is independently at each occurrence selected from C₃-C₆-cycloalkyl and 4- to 6-membered heterocycloalkyl.

In some embodiments, R⁴ is independently at each occurrence selected from cyano, C₀-C₄-alkylene-NR⁵R⁶, C₀-C₄-alkylene-OR⁷, S(O)₂RC, C₁-C₄-alkyl, 4- to 6-membered heterocycloalkyl, C₀-C₄-alkylene-S(O)₂R⁶, and C₁-C₄-haloalkyl.

In some embodiments, R⁴ is independently at each occurrence selected from cyano, C₀-C₄-alkylene-NR⁵R⁶, C₀-C₄-alkylene-OR⁷, S(O)₂R⁶, C₁-C₄-alkyl, CO—C₄-alkylene-S(O)₂R⁶, and C₁-C₄-haloalkyl.

In some embodiments, R⁴ is independently at each occurrence selected from cyano, C₀-C₄-alkylene-NR⁵R⁶, C₀-C₄-alkylene-OR⁷, S(O)₂R⁶, C₁-C₂-alkyl, 4-membered heterocycloalkyl, C(CH₃)₂OH, C₁-C₂-alkyl-S(O)₂R⁶, and C₁-C₂-haloalkyl.

In some embodiments, R⁴ is independently at each occurrence selected from cyano, C₀-C₄-alkylene-NR⁵R⁶, C₀-C₄-alkylene-OR⁷, S(O)₂R⁶, C₁-C₂-alkyl, C(CH₃)₂OH, C₁-C₂-alkyl-S(O)₂R⁶, and C₁-C₂-haloalkyl.

In some embodiments, R⁴ is independently at each occurrence selected from C₀-C₄-alkylene-NR⁵R⁶, S(O)₂R⁶, C₁-alkyl, 4-membered heterocycloalkyl, C(CH₃)₂OH, C₁-alkyl-S(O)₂R⁶, and C₁-haloalkyl.

In some embodiments, R⁴ is independently at each occurrence selected from C₀-C₄-alkylene-NR⁵R⁶, S(O)₂R⁶, C₁-alkyl, C(CH₃)₂OH, C₁-alkyl-S(O)₂R⁶, and C₁-haloalkyl.

In some embodiments, R⁴ is independently at each occurrence selected from cyano, NR⁵R⁶, OR⁶, S(O)₂R⁶, C₁-C₂-alkyl, 4-membered heterocycloalkyl, C(CH₃)₂OH, C₁-C₂-alkyl-S(O)₂R⁶, and C₁-C₂-haloalkyl.

In some embodiments, R⁴ is independently at each occurrence selected from cyano, NR⁵R⁶, OR⁷, S(O)₂R⁶, C₁-C₂-alkyl, C(CH₃)₂OH, C₁-C₂-alkyl-S(O)₂R⁶, and C₁-C₂-haloalkyl.

In some embodiments, R⁴ is independently at each occurrence selected from NR⁵R⁶, S(O)₂R⁶, C₁-alkyl, oxetanyl (e.g., oxetan-3-yl), C(CH₃)₂OH, C₁-alkyl-S(O)₂R⁶, and C₁-haloalkyl.

In some embodiments, R⁴ is independently at each occurrence selected from NR⁵R⁶, S(O)₂R⁶, C₁-alkyl, C(CH₃)₂OH, C₁-alkyl-S(O)₂R⁶, and C₁-haloalkyl.

In some embodiments, R⁴ is independently at each occurrence selected from N(H)S(O)₂Me, S(O)₂MeR⁶, C(CH₃)₂OH, C₁-alkyl-S(O)₂Me.

In some embodiments, m is an integer selected from 0, 1, and 2. In some embodiments, m is 2. In some embodiments, m is 1. In some embodiments, m is 0.

In some embodiments, R^{4a} is H. In some embodiments, R^{4a} is methyl. In some embodiments, R^{4a} is cyclopropyl. In some embodiments, R^{4a} is C₀-C₄-alkylene-R^{4c}. In some embodiments, R^{4c} is independently at each occurrence selected from C₃-C₆-cycloalkyl and 4- to 6-membered heterocycloalkyl. In some embodiments, R⁴⁸ is a 4-membered heterocycloalkyl. In some embodiments, R^{4a} is oxetanyl. In some embodiments, R^{4a} is oxetan-3-yl. In some embodiments, R^{4a} is oxetanyl or azetidinyl. In some embodiments, R^{4a} is independently selected from H, C₁-C₄-alkyl and cyclopropyl. In some embodiments, R^{4a} is independently selected from C₁-C₄-alkyl, cyclopropyl and cyclobutyl. In an embodiment R^{4a} is cyclopropyl.

In some embodiments, R^{4b} is selected from S(O)₂R⁶. In some embodiments, R^{4b} is C₁-C₄-alkyl. In some embodiments, R^{4b} is C₁-C₄-alkyl-S(O)₂R¹. In some embodiments, R^{4b} is C₁-C₄-haloalkyl. In some embodiments, R^{4b} is cyclopropyl.

In some embodiments, R^{4b} is selected from S(O)₂—C₁-C₃-alkyl, e.g., S(O)₂Me. In some embodiments, R^{4b} is C₁-C₄-alkyl, e.g., methyl. In some embodiments, R^{4b} is C₁-C₄-alkyl-S(O)₂—C₁-C₄-alkyl, e.g., —CH₂—S(O)₂-Me.

In some embodiments, R^{4c} is C₃-C₆-cycloalkyl. In some embodiments, R^{4c} is 4- to 6-membered heterocycloalkyl.

In some embodiments, R⁵ is independently at each occurrence selected from H, C₁-C₄-alkyl, and S(O)₂—C₁-C₄-alkyl.

In some embodiments, R⁵ is S(O)₂—C₁-C₄-alkyl; optionally wherein R⁵ is S(O)₂—C₁-alkyl. In some embodiments, R⁸ is H. In some embodiments, R⁵ is methyl.

In some embodiments, R⁶ is independently at each occurrence selected from H and C₁-C₄-alkyl. In some embodiments, R⁶ is H. In some embodiments, R⁶ is methyl.

In some embodiments, R⁷ is independently at each occurrence selected from H, C₁-C₄-alkyl, and C₁-C₄-haloalkyl.

In some embodiments, R⁷ is independently at each occurrence selected from H, and C₁-C₄-alkyl.

In some embodiments, R⁷ is independently at each occurrence selected from H, C₁-C₂-alkyl, and C₁-C₂-haloalkyl.

In some embodiments, R⁷ is independently at each occurrence selected from H, and C₁-C₂-alkyl.

In some embodiments, R⁷ is independently at each occurrence H.

In some embodiments, R⁶ is independently at each occurrence selected from ═O, fluoro, nitro, cyano, NR⁶R⁶, OR⁷, C(O)R⁶, C₁-C₄-alkyl, C₁-C₄-haloalkyl and cyclopropyl.

In some embodiments, R⁸ is independently at each occurrence selected from ═O, fluoro, C(O)R⁶, C₁-C₂-alkyl, and C₁-C₂-haloalkyl.

In some embodiments, R⁸ is independently at each occurrence selected from ═O, fluoro, and C(O)R⁶. In some embodiments, R⁶ is independently at each occurrence selected from ═O, fluoro, and C(O)Me.

In some embodiments, R⁹ is independently at each occurrence selected from halo, nitro, cyano, C₀-C₄-alkylene-OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl and C₁-C₄-haloalkyl.

In some embodiments, R⁹ is independently at each occurrence selected from halo, nitro, cyano, NR⁵R⁶, OR⁷, C(O)R⁶, C₁-C₄-alkyl, C₁-C₄-haloalkyl and cyclopropyl.

In some embodiments, R⁹ is independently at each occurrence selected from halo, C₁-C₄-alkyl, and C₁-C₄-haloalkyl.

In some embodiments, R⁹ is independently at each occurrence selected from halo, C₁-C₂-alkyl, and C₁-C₂-haloalkyl.

In some embodiments, R⁹ is independently at each occurrence selected from halo, and C₁-C₂-alkyl. In some embodiments, R⁹ is independently at each occurrence selected from fluoro and methyl.

In some embodiments, R^{x} and R^{y} are each independently selected from H, halo, nitro, cyano, NR⁵R⁶, OR⁷, SR⁶, C₁-C₄-alkyl, C₁-C₄-haloalkyl and C₃-C₄-cycloalkyl.

In some embodiments, R^{x} and R^{y} are each independently selected from H, halo, cyano, C₁-C₂-alkyl, C₁-C₂-haloalkyl and C₃-C₄cycloalkyl.

In some embodiments, R^{x} is H. In some embodiments, R^{Y} is H. In some embodiments, R^{x} and R^{y} are each H.

In some embodiments, any of the alkyl or alkenyl groups are optionally substituted, where chemically possible, by 1 to 5 substituents which are each independently at each occurrence selected from the group consisting of: oxo, fluoro, NR^{a}R^{b}, OR^{a}, and S(O)₂R^{a}, wherein R^{a} is independently at each occurrence selected from H, and C₁-C₄-alkyl; and R^{b} is independently at each occurrence selected from H, C₁-C₄-alkyl, C(O)—C₁-C₄-alkyl and S(O)₂—C₁-C₄-alkyl.

In some embodiments, X⁶ is carbon. In some embodiments, X⁶ is nitrogen.

In some embodiments, X⁷ is carbon. In some embodiments, X⁷ is nitrogen.

In some embodiments, p is an integer selected from 0, 1, 2, and 3. In some embodiments, p is 3. In some embodiments, p is 2. In some embodiments, p is 1. In some embodiments, p is 0.

In some embodiments, the compound of formula (IA) is a compound of formula (XXI), a tautomer, a stereoisomer or a mixture of stereoisomers, a pharmaceutically acceptable salt, a hydrate, a deuterated derivative, or an N-oxide thereof:

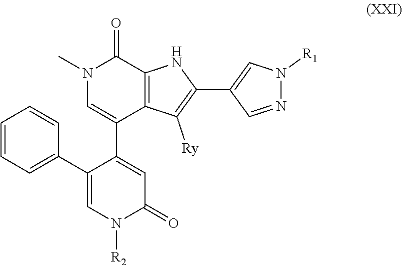

wherein:
R₁ is C₁-C₄-haloalkyl;
R₂ is C₁-C₄-alkyl, wherein the C₁-C₄-alkyl is optionally substituted with SR₃ or OR₃;
R₃ is selected from H, C₁-C₃-alkyl, C(O)—C₁-C₃-alkyl, and C₁-C₃-haloalkyl; and
R_{y} is hydrogen or halo.

In some embodiments, Ri is independently selected from CF₃, CHF₂, CH₂CF₃, and CH₂CH₂F. In some embodiments, Ri is independently selected from CBr₃, CHBr₂, CH₂CBr₃, and CH₂CH₂Br. In some embodiments, R¹ is independently selected from CCl₃, CHCl₂, CH₂CCl₃, and CH₂CH₂Cl. In some embodiments, R₁ is CH₂CH₂F. In some embodiments, R¹ is CHF₂. In some embodiments, R₁ is CF₃.

In some embodiments, R₂ is selected from —CH₃, —CH₂—CH₂—S—CH₃, —CH₂—CH₂—O—CH₃, —CH₂—CH₂—SCH₂—CH₃, and —CH₂—CH₂—OCH₂—CH₃. In some embodiments, R₂ is —CH₃. In some embodiments, R₂ is —CH₂—CH₂—O—CH₃. In some embodiments, R₂ is —CH₂—CH₂—S—CH₃.

In some embodiments, R₂ is C₁-C₄-alkyl, wherein the C₁-C₄-alkyl is substituted with OR₃. In some embodiments, R₂ is C₁-C₄-alkyl, wherein the C₁-C₄-alkyl is substituted with SR₃. In some embodiments, R₂ is —CH₃.

In some embodiments, R_{y} is halo. In some embodiments, R_{y} is fluorine.

In some embodiments, the compound of Formula (XXI) is a compound of Formula (XXIA), a tautomer, a stereoisomer or a mixture of stereoisomers, a pharmaceutically acceptable salt, a hydrate, a deuterated derivative, or an N-oxide thereof:

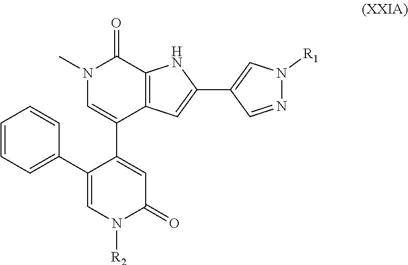

wherein:
R¹ is C₁-C₄-haloalkyl;
R₂ is C₁-C₄-alkyl, wherein the C₁-C₄-alkyl is optionally substituted with SR₃ or OR₃; and
R₃ is selected from H, C₁-C₃-alkyl, C(O)—C₁-C₃-alkyl, and C₁-C₃-haloalkyl.

In some embodiments, R¹ is independently selected from CF₃, CHF₂, CH₂CF₃, and CH₂CH₂F. In some embodiments, R¹ is independently selected from CBr₃, CHBr₂, CH₂CBr₃, and CH₂CH₂Br. In some embodiments, R¹ is independently selected from CCl₃, CHCl₂, CH₂CCl₃, and CH₂CH₂Cl. In some embodiments, R¹ is CH₂CH₂F. In some embodiments, R¹ is CHF₂. In some embodiments, R¹ is CF₃.

In some embodiments, R₂ is C₁-C₄-alkyl, wherein the C₁-C₄-alkyl is substituted with OR₃. In some embodiments, R₂ is C₁-C₄-alkyl, wherein the C₁-C₄-alkyl is substituted with SR₃. In some embodiments, R₂ is selected from —CH₃, —CH₂—CH₂—O—CH₃, and —CH₂—CH₂—OCH₂—CH₃. In some embodiments, R₂ is selected from —CH₃, —CH₂—CH₂—S—CH₃, and —CH₂—CH₂—SCH₂—CH₃.

In some embodiments, R₂ is —CH₃. In some embodiments, R₂ is —CH₂—CH₂—O—CH₃. In some embodiments, R₂ is —CH₂—CH₂—S—CH₃.

In some embodiments, the compound of Formula (XXI) is a compound of Formula (XXIB), a tautomer, a stereoisomer or a mixture of stereoisomers, a pharmaceutically acceptable salt, a hydrate, a deuterated derivative, or an N-oxide thereof:

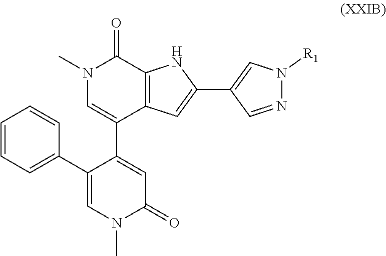

wherein:
R₁ is C₁-C₄-haloalkyl.

In some embodiments, R₁ is independently selected from CF₃, CHF₂, CH₂CF₃, and CH₂CH₂F. In some embodiments, R₁ is independently selected from CBr₃, CHBr₂, CH₂CBr₃, and CH₂CH₂Br. In some embodiments, R₁ is independently selected from CCl₃, CHCl₂, CH₂CCl₃, and CH₂CH₂Cl. In some embodiments, R₁ is CH₂CH₂F. In some embodiments, R₁ is CHF₂. In some embodiments, R₁ is CF₃.

In some embodiments, the compound is selected from:

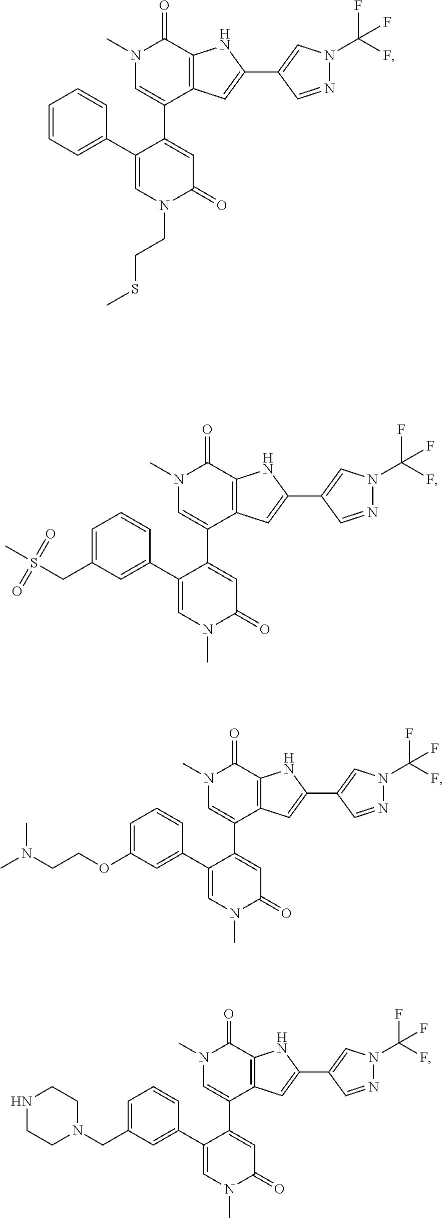 [SMILES: CSCCn1cc(-c2ccccc2)c(-c2cn(C)c(=O)c3[nH]c(-c4cnn(C(F)(F)F)c4)cc23)cc1=O ; Cn1cc(-c2cccc(CS(C)(=O)=O)c2)c(-c2cn(C)c(=O)c3[nH]c(-c4cnn(C(F)(F)F)c4)cc23)cc1=O ; CN(C)CCOc1cccc(-c2cn(C)c(=O)cc2-c2cn(C)c(=O)c3[nH]c(-c4cnn(C(F)(F)F)c4)cc23)c1 ; Cn1cc(-c2cccc(CN3CCNCC3)c2)c(-c2cn(C)c(=O)c3[nH]c(-c4cnn(C(F)(F)F)c4)cc23)cc1=O]

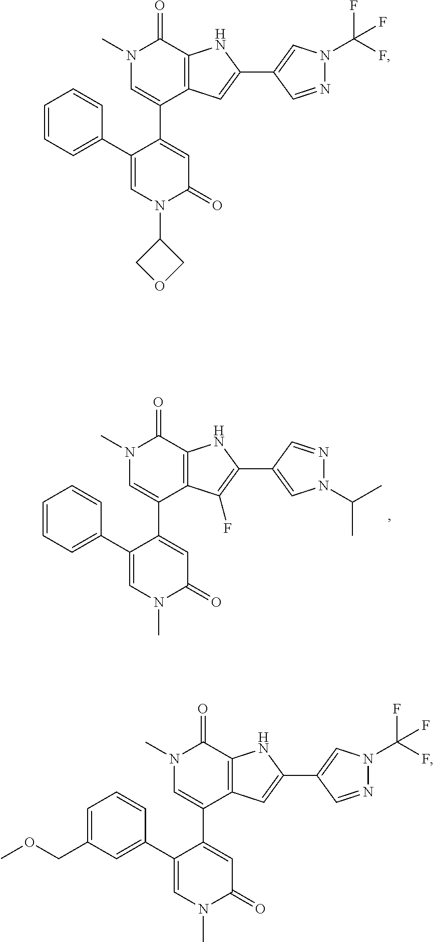

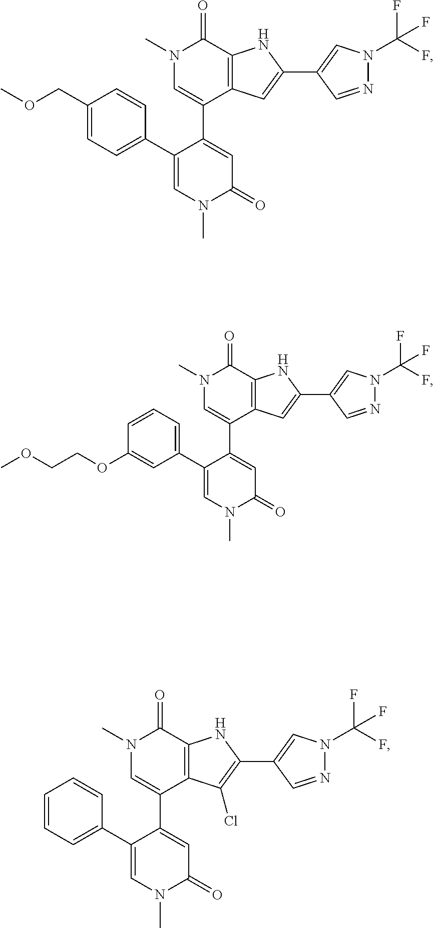 [SMILES: COCc1ccc(-c2cn(C)c(=O)cc2-c2cn(C)c(=O)c3[nH]c(-c4cnn(C(F)(F)F)c4)cc23)cc1 ; COCCOc1cccc(-c2cn(C)c(=O)cc2-c2cn(C)c(=O)c3[nH]c(-c4cnn(C(F)(F)F)c4)cc23)c1 ; Cn1cc(-c2ccccc2)c(-c2cn(C)c(=O)c3[nH]c(-c4cnn(C(F)(F)F)c4)c(Cl)c23)cc1=O]

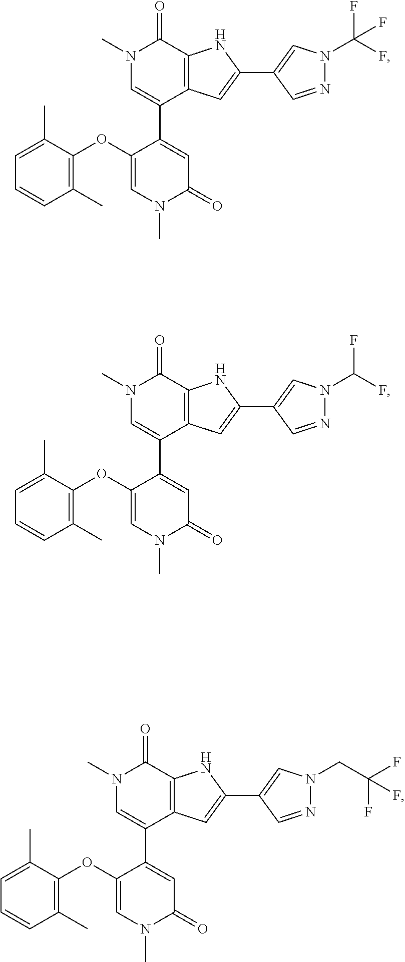 [SMILES: Cc1cccc(C)c1Oc1cn(C)c(=O)cc1-c1cn(C)c(=O)c2[nH]c(-c3cnn(C(F)(F)F)c3)cc12 ; Cc1cccc(C)c1Oc1cn(C)c(=O)cc1-c1cn(C)c(=O)c2[nH]c(-c3cnn(C(F)F)c3)cc12 ; Cc1cccc(C)c1Oc1cn(C)c(=O)cc1-c1cn(C)c(=O)c2[nH]c(-c3cnn(CC(F)(F)F)c3)cc12]

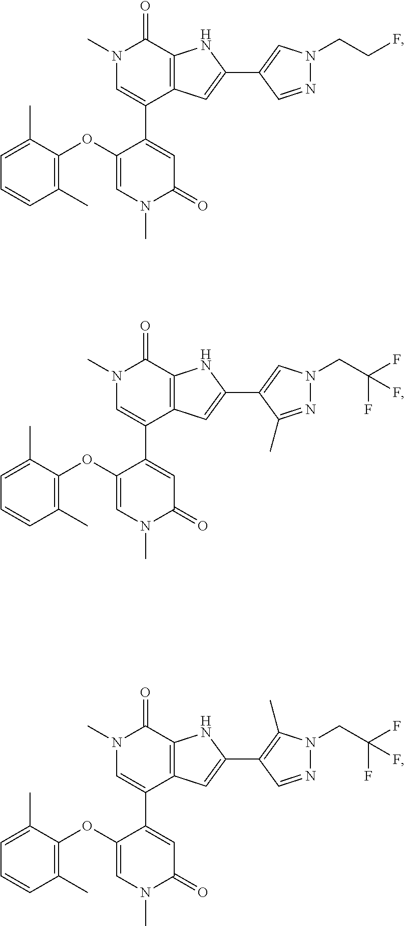 [SMILES: Cc1cccc(C)c1Oc1cn(C)c(=O)cc1-c1cn(C)c(=O)c2[nH]c(-c3cnn(CCF)c3)cc12 ; Cc1cccc(C)c1Oc1cn(C)c(=O)cc1-c1cn(C)c(=O)c2[nH]c(-c3cn(CC(F)(F)F)nc3C)cc12 ; Cc1cccc(C)c1Oc1cn(C)c(=O)cc1-c1cn(C)c(=O)c2[nH]c(-c3cnn(CC(F)(F)F)c3C)cc12]

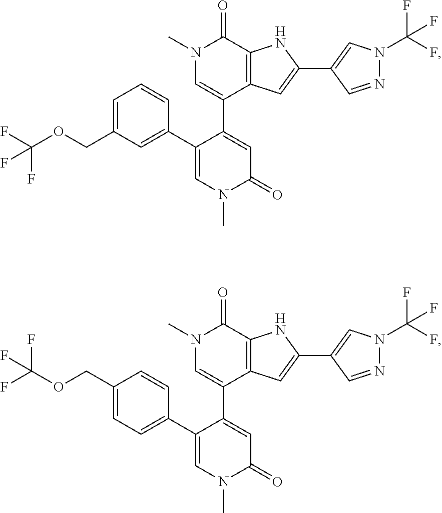 [SMILES: Cn1cc(-c2cccc(COC(F)(F)F)c2)c(-c2cn(C)c(=O)c3[nH]c(-c4cnn(C(F)(F)F)c4)cc23)cc1=O ; Cn1cc(-c2ccc(COC(F)(F)F)cc2)c(-c2cn(C)c(=O)c3[nH]c(-c4cnn(C(F)(F)F)c4)cc23)cc1=O]

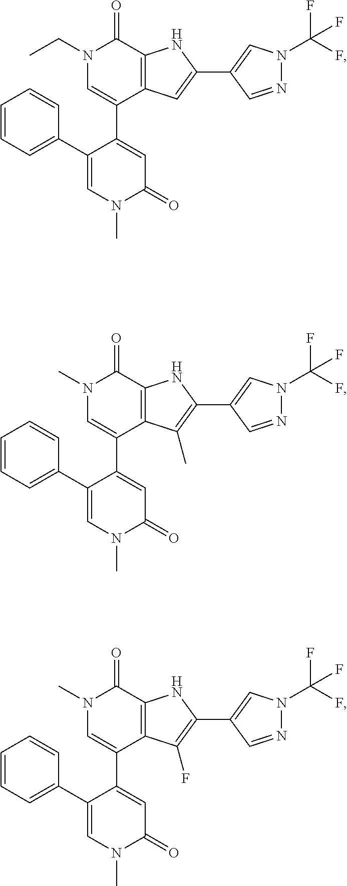 [SMILES: CCn1cc(-c2cc(=O)n(C)cc2-c2ccccc2)c2cc(-c3cnn(C(F)(F)F)c3)[nH]c2c1=O ; Cc1c(-c2cnn(C(F)(F)F)c2)[nH]c2c(=O)n(C)cc(-c3cc(=O)n(C)cc3-c3ccccc3)c12 ; Cn1cc(-c2ccccc2)c(-c2cn(C)c(=O)c3[nH]c(-c4cnn(C(F)(F)F)c4)c(F)c23)cc1=O]

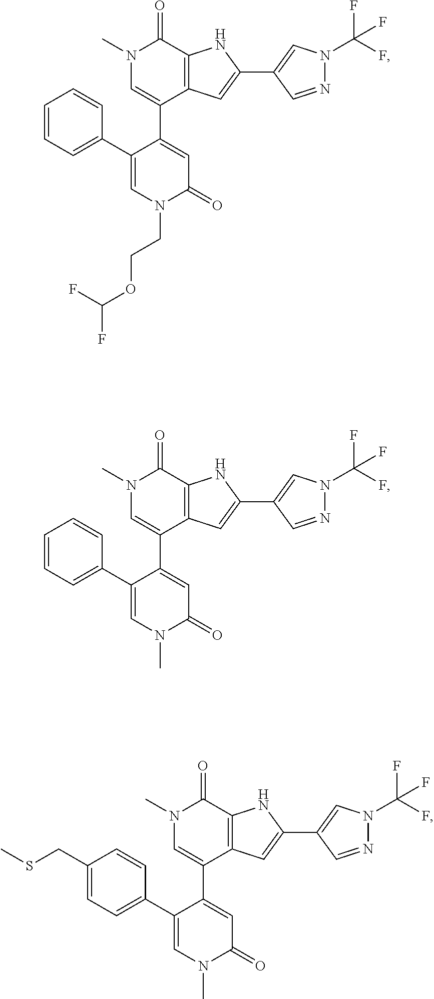 [SMILES: Cn1cc(-c2cc(=O)n(CCOC(F)F)cc2-c2ccccc2)c2cc(-c3cnn(C(F)(F)F)c3)[nH]c2c1=O ; Cn1cc(-c2ccccc2)c(-c2cn(C)c(=O)c3[nH]c(-c4cnn(C(F)(F)F)c4)cc23)cc1=O ; CSCc1ccc(-c2cn(C)c(=O)cc2-c2cn(C)c(=O)c3[nH]c(-c4cnn(C(F)(F)F)c4)cc23)cc1]

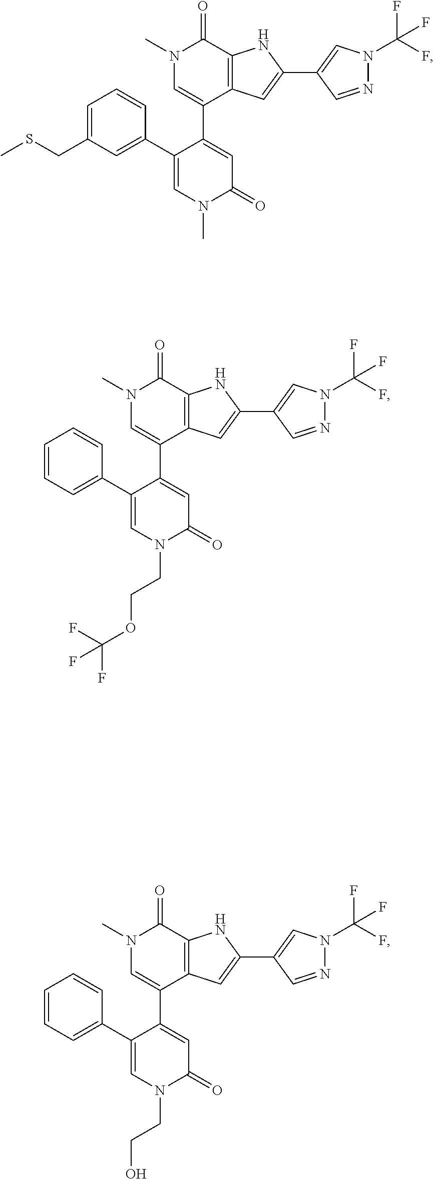 [SMILES: CSCc1cccc(-c2cn(C)c(=O)cc2-c2cn(C)c(=O)c3[nH]c(-c4cnn(C(F)(F)F)c4)cc23)c1 ; Cn1cc(-c2cc(=O)n(CCOC(F)(F)F)cc2-c2ccccc2)c2cc(-c3cnn(C(F)(F)F)c3)[nH]c2c1=O ; Cn1cc(-c2cc(=O)n(CCO)cc2-c2ccccc2)c2cc(-c3cnn(C(F)(F)F)c3)[nH]c2c1=O]

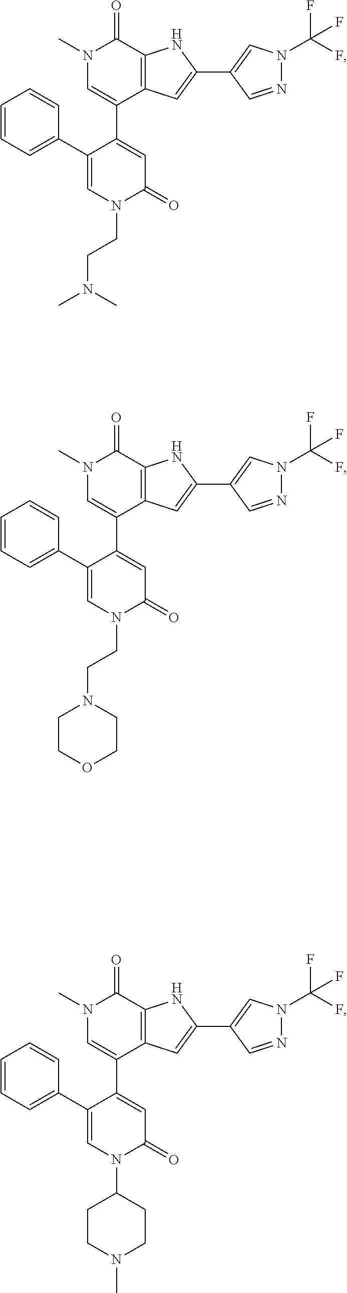 [SMILES: CN(C)CCn1cc(-c2ccccc2)c(-c2cn(C)c(=O)c3[nH]c(-c4cnn(C(F)(F)F)c4)cc23)cc1=O ; Cn1cc(-c2cc(=O)n(CCN3CCOCC3)cc2-c2ccccc2)c2cc(-c3cnn(C(F)(F)F)c3)[nH]c2c1=O ; CN1CCC(n2cc(-c3ccccc3)c(-c3cn(C)c(=O)c4[nH]c(-c5cnn(C(F)(F)F)c5)cc34)cc2=O)CC1]

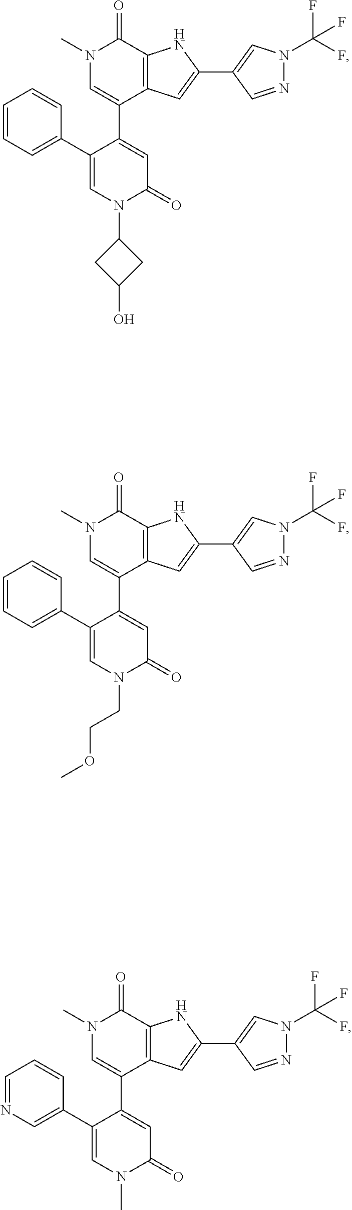 [SMILES: Cn1cc(-c2cc(=O)n(C3CC(O)C3)cc2-c2ccccc2)c2cc(-c3cnn(C(F)(F)F)c3)[nH]c2c1=O ; COCCn1cc(-c2ccccc2)c(-c2cn(C)c(=O)c3[nH]c(-c4cnn(C(F)(F)F)c4)cc23)cc1=O ; Cn1cc(-c2cccnc2)c(-c2cn(C)c(=O)c3[nH]c(-c4cnn(C(F)(F)F)c4)cc23)cc1=O]

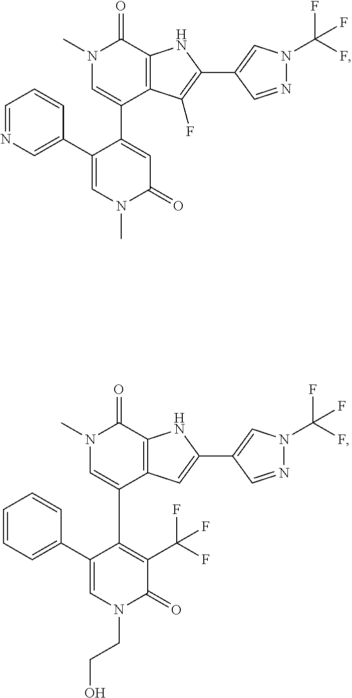 [SMILES: Cn1cc(-c2cccnc2)c(-c2cn(C)c(=O)c3[nH]c(-c4cnn(C(F)(F)F)c4)c(F)c23)cc1=O ; Cn1cc(-c2c(-c3ccccc3)cn(CCO)c(=O)c2C(F)(F)F)c2cc(-c3cnn(C(F)(F)F)c3)[nH]c2c1=O]

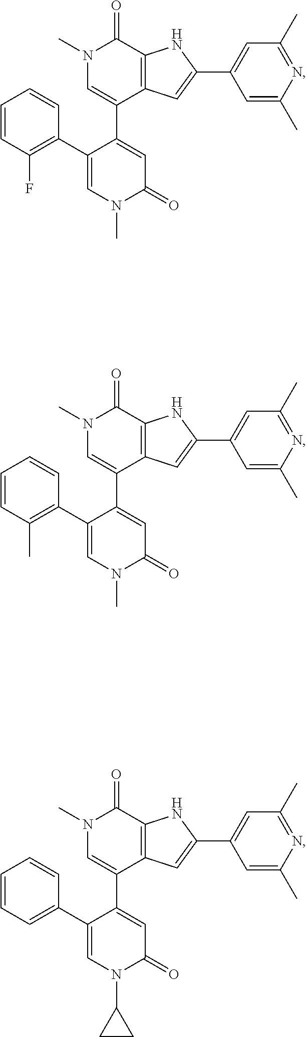 [SMILES: Cc1cc(-c2cc3c(-c4cc(=O)n(C)cc4-c4ccccc4F)cn(C)c(=O)c3[nH]2)cc(C)n1 ; Cc1cc(-c2cc3c(-c4cc(=O)n(C)cc4-c4ccccc4C)cn(C)c(=O)c3[nH]2)cc(C)n1 ; Cc1cc(-c2cc3c(-c4cc(=O)n(C5CC5)cc4-c4ccccc4)cn(C)c(=O)c3[nH]2)cc(C)n1]

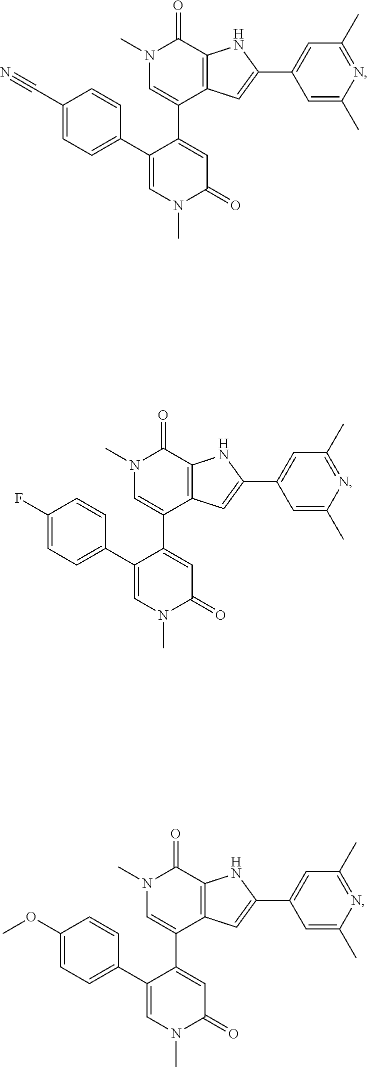

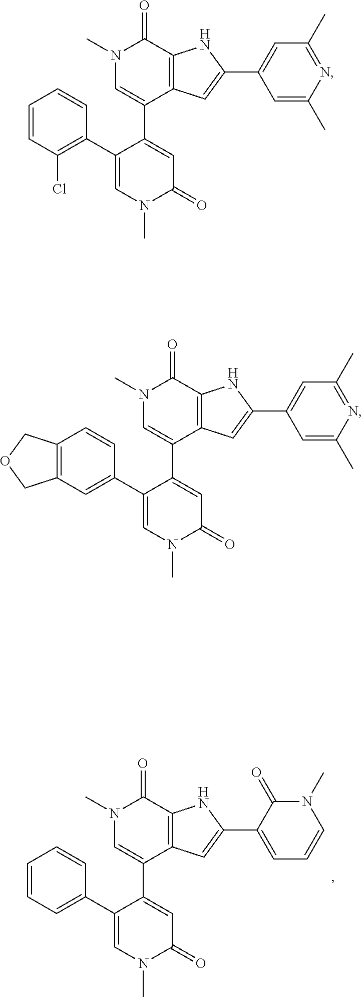 [SMILES: Cc1cc(-c2cc3c(-c4cc(=O)n(C)cc4-c4ccccc4Cl)cn(C)c(=O)c3[nH]2)cc(C)n1 ; Cc1cc(-c2cc3c(-c4cc(=O)n(C)cc4-c4ccc5c(c4)COC5)cn(C)c(=O)c3[nH]2)cc(C)n1 ; Cn1cc(-c2ccccc2)c(-c2cn(C)c(=O)c3[nH]c(-c4cccn(C)c4=O)cc23)cc1=O]

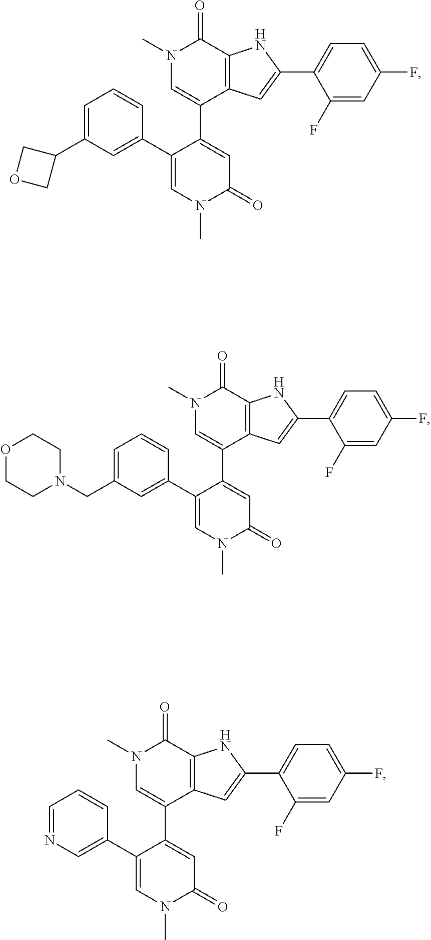 [SMILES: Cn1cc(-c2cccc(C3COC3)c2)c(-c2cn(C)c(=O)c3[nH]c(-c4ccc(F)cc4F)cc23)cc1=O ; Cn1cc(-c2cccc(CN3CCOCC3)c2)c(-c2cn(C)c(=O)c3[nH]c(-c4ccc(F)cc4F)cc23)cc1=O ; Cn1cc(-c2cccnc2)c(-c2cn(C)c(=O)c3[nH]c(-c4ccc(F)cc4F)cc23)cc1=O]

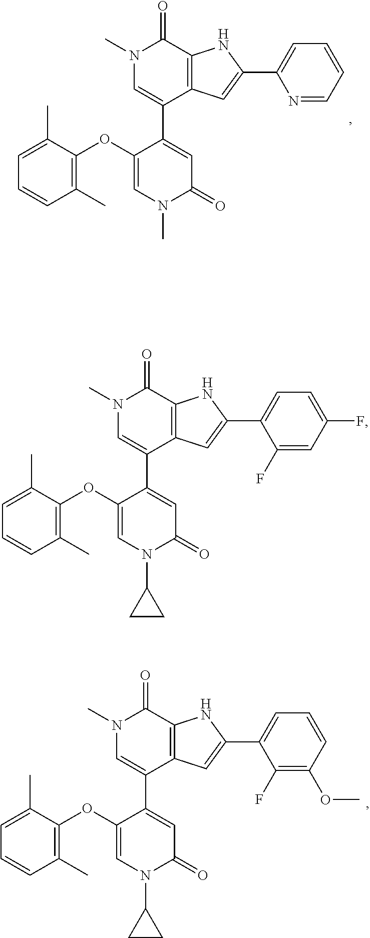 [SMILES: Cc1cccc(C)c1Oc1cn(C)c(=O)cc1-c1cn(C)c(=O)c2[nH]c(-c3ccccn3)cc12 ; Cc1cccc(C)c1Oc1cn(C2CC2)c(=O)cc1-c1cn(C)c(=O)c2[nH]c(-c3ccc(F)cc3F)cc12 ; COc1cccc(-c2cc3c(-c4cc(=O)n(C5CC5)cc4Oc4c(C)cccc4C)cn(C)c(=O)c3[nH]2)c1F]

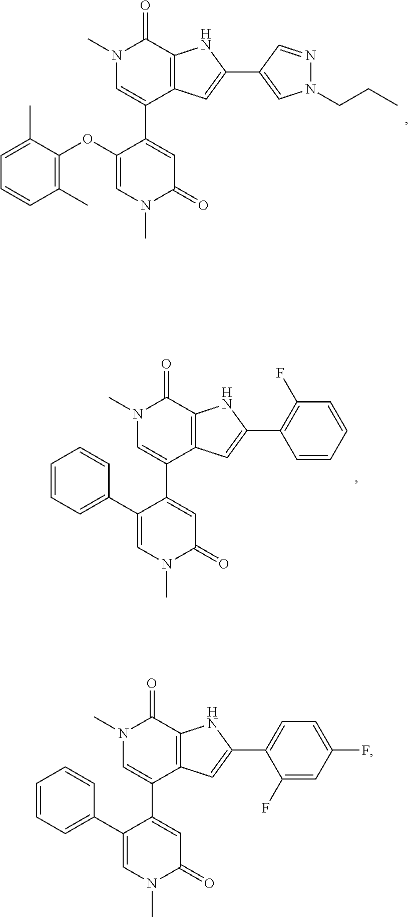 [SMILES: CCCn1cc(-c2cc3c(-c4cc(=O)n(C)cc4Oc4c(C)cccc4C)cn(C)c(=O)c3[nH]2)cn1 ; Cn1cc(-c2ccccc2)c(-c2cn(C)c(=O)c3[nH]c(-c4ccccc4F)cc23)cc1=O ; Cn1cc(-c2ccccc2)c(-c2cn(C)c(=O)c3[nH]c(-c4ccc(F)cc4F)cc23)cc1=O]

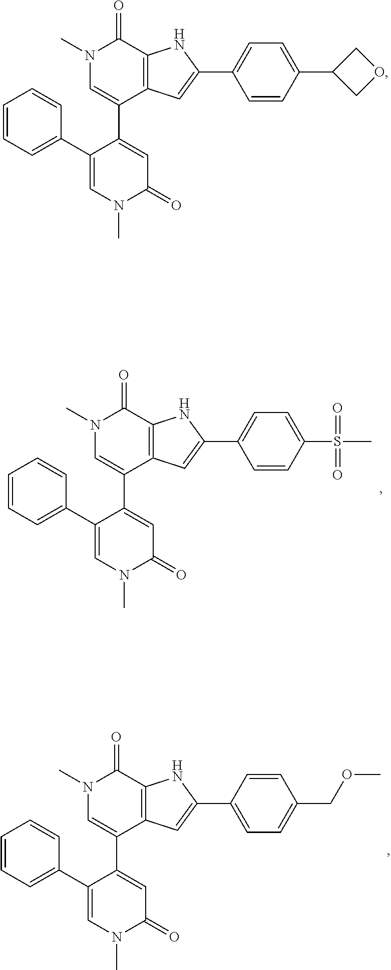 [SMILES: Cn1cc(-c2ccccc2)c(-c2cn(C)c(=O)c3[nH]c(-c4ccc(C5COC5)cc4)cc23)cc1=O ; Cn1cc(-c2ccccc2)c(-c2cn(C)c(=O)c3[nH]c(-c4ccc(S(C)(=O)=O)cc4)cc23)cc1=O ; COCc1ccc(-c2cc3c(-c4cc(=O)n(C)cc4-c4ccccc4)cn(C)c(=O)c3[nH]2)cc1]

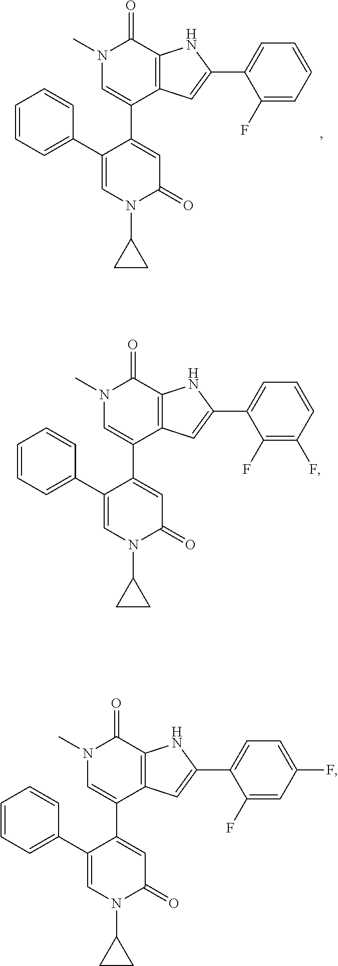 [SMILES: Cn1cc(-c2cc(=O)n(C3CC3)cc2-c2ccccc2)c2cc(-c3ccccc3F)[nH]c2c1=O ; Cn1cc(-c2cc(=O)n(C3CC3)cc2-c2ccccc2)c2cc(-c3cccc(F)c3F)[nH]c2c1=O ; Cn1cc(-c2cc(=O)n(C3CC3)cc2-c2ccccc2)c2cc(-c3ccc(F)cc3F)[nH]c2c1=O]

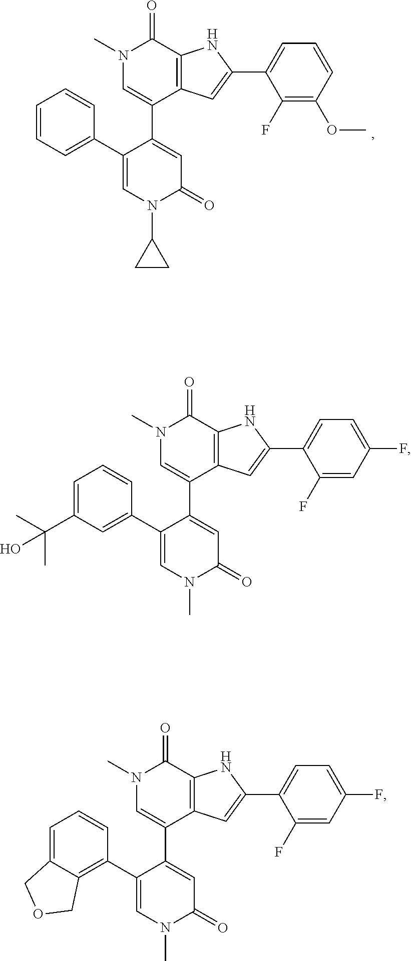

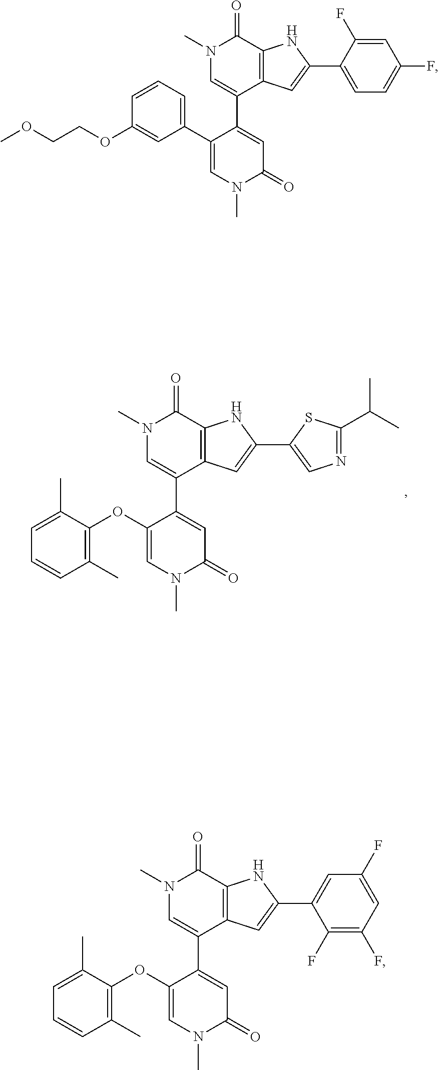 [SMILES: COCCOc1cccc(-c2cn(C)c(=O)cc2-c2cn(C)c(=O)c3[nH]c(-c4ccc(F)cc4F)cc23)c1 ; Cc1cccc(C)c1Oc1cn(C)c(=O)cc1-c1cn(C)c(=O)c2[nH]c(-c3cnc(C(C)C)s3)cc12 ; Cc1cccc(C)c1Oc1cn(C)c(=O)cc1-c1cn(C)c(=O)c2[nH]c(-c3cc(F)cc(F)c3F)cc12]

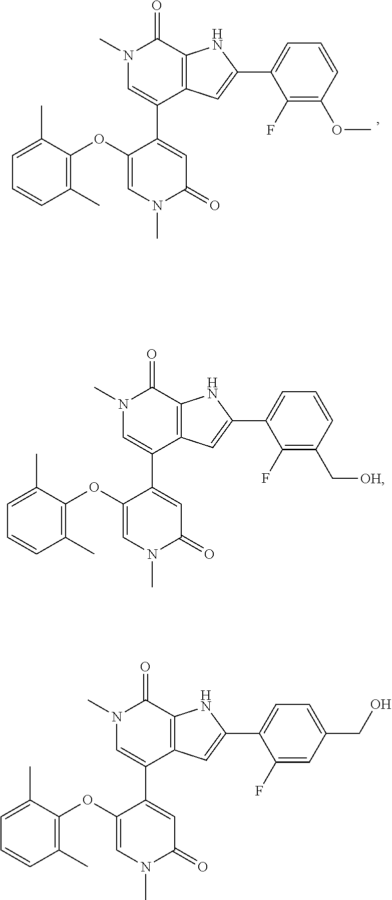 [SMILES: COc1cccc(-c2cc3c(-c4cc(=O)n(C)cc4Oc4c(C)cccc4C)cn(C)c(=O)c3[nH]2)c1F ; Cc1cccc(C)c1Oc1cn(C)c(=O)cc1-c1cn(C)c(=O)c2[nH]c(-c3cccc(CO)c3F)cc12 ; Cc1cccc(C)c1Oc1cn(C)c(=O)cc1-c1cn(C)c(=O)c2[nH]c(-c3ccc(CO)cc3F)cc12]

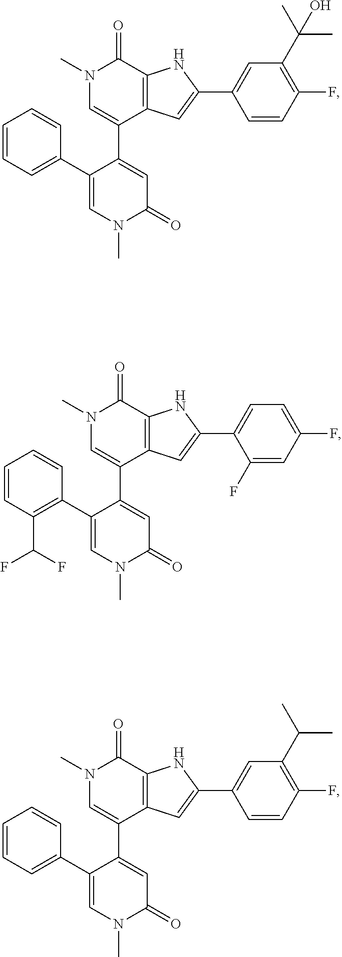 [SMILES: Cn1cc(-c2ccccc2)c(-c2cn(C)c(=O)c3[nH]c(-c4ccc(F)c(C(C)(C)O)c4)cc23)cc1=O ; Cn1cc(-c2ccccc2C(F)F)c(-c2cn(C)c(=O)c3[nH]c(-c4ccc(F)cc4F)cc23)cc1=O ; CC(C)c1cc(-c2cc3c(-c4cc(=O)n(C)cc4-c4ccccc4)cn(C)c(=O)c3[nH]2)ccc1F]

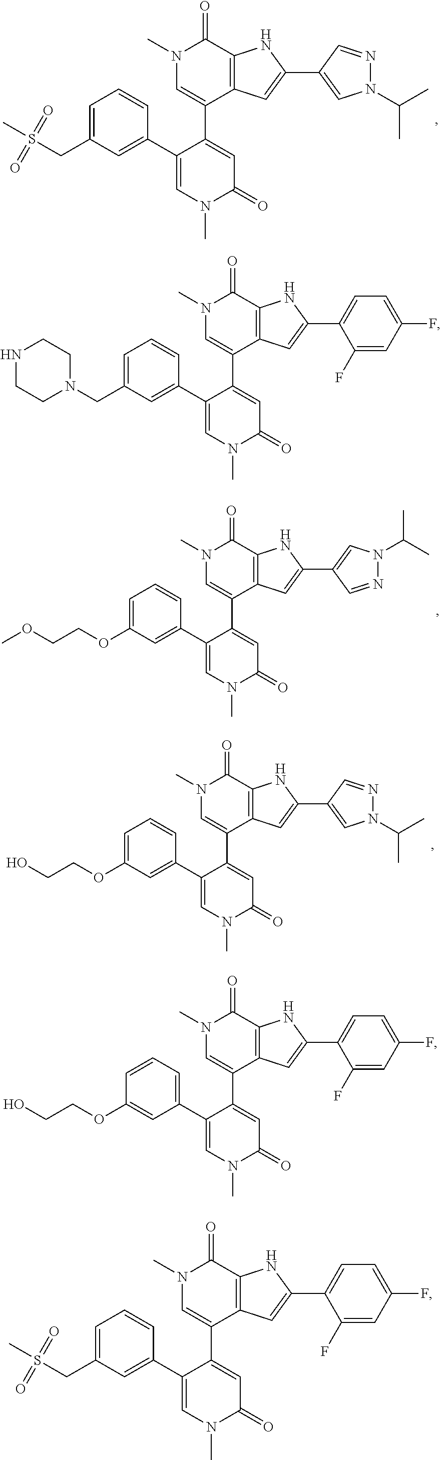

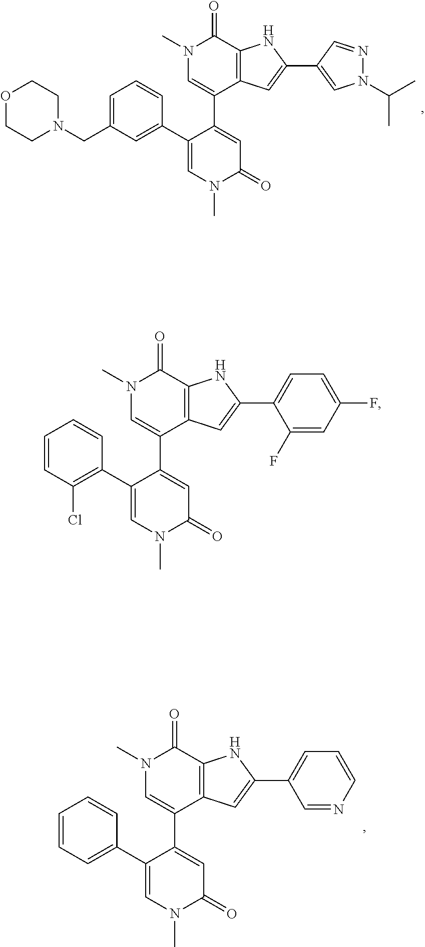 [SMILES: CC(C)n1cc(-c2cc3c(-c4cc(=O)n(C)cc4-c4cccc(CN5CCOCC5)c4)cn(C)c(=O)c3[nH]2)cn1 ; Cn1cc(-c2ccccc2Cl)c(-c2cn(C)c(=O)c3[nH]c(-c4ccc(F)cc4F)cc23)cc1=O ; Cn1cc(-c2ccccc2)c(-c2cn(C)c(=O)c3[nH]c(-c4cccnc4)cc23)cc1=O]

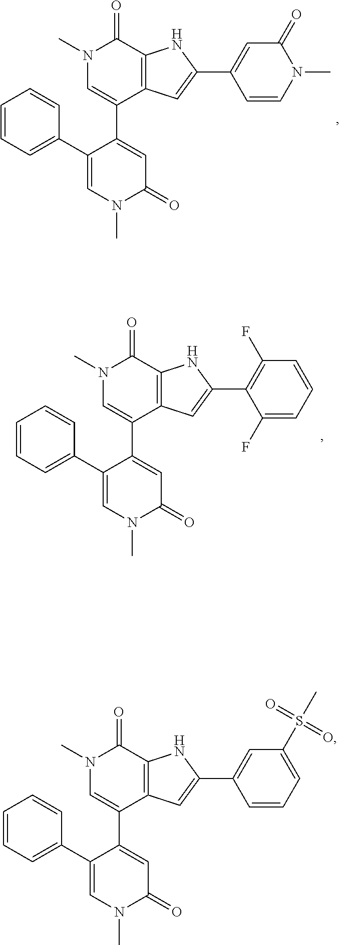 [SMILES: Cn1ccc(-c2cc3c(-c4cc(=O)n(C)cc4-c4ccccc4)cn(C)c(=O)c3[nH]2)cc1=O ; Cn1cc(-c2ccccc2)c(-c2cn(C)c(=O)c3[nH]c(-c4c(F)cccc4F)cc23)cc1=O ; Cn1cc(-c2ccccc2)c(-c2cn(C)c(=O)c3[nH]c(-c4cccc(S(C)(=O)=O)c4)cc23)cc1=O]

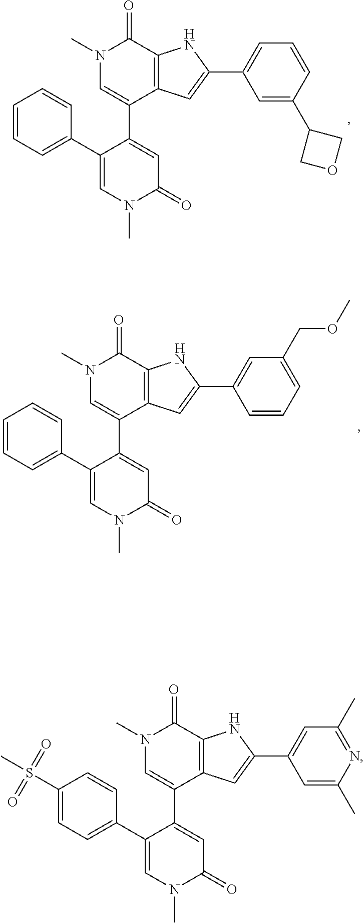

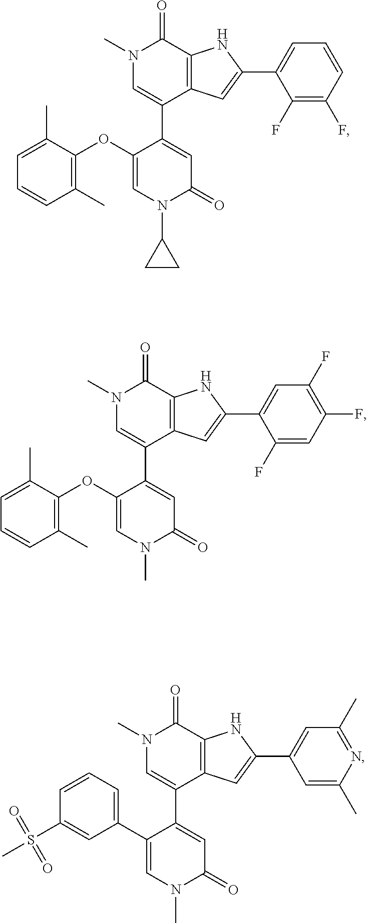

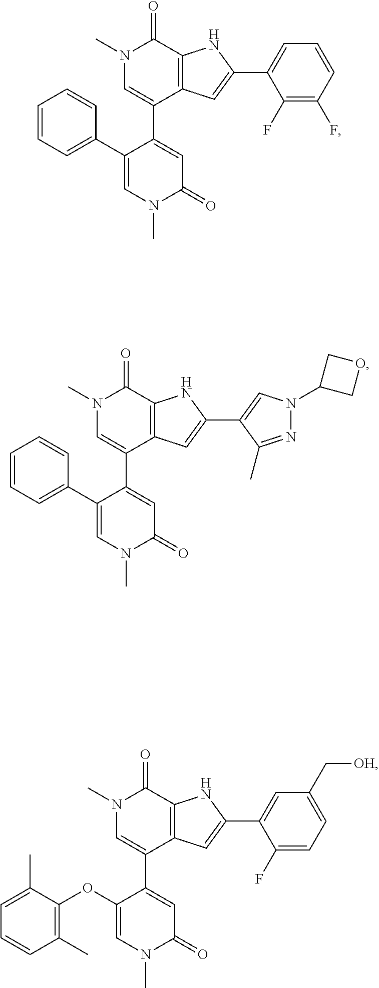 [SMILES: Cn1cc(-c2ccccc2)c(-c2cn(C)c(=O)c3[nH]c(-c4cccc(F)c4F)cc23)cc1=O ; Cc1nn(C2COC2)cc1-c1cc2c(-c3cc(=O)n(C)cc3-c3ccccc3)cn(C)c(=O)c2[nH]1 ; Cc1cccc(C)c1Oc1cn(C)c(=O)cc1-c1cn(C)c(=O)c2[nH]c(-c3cc(CO)ccc3F)cc12]

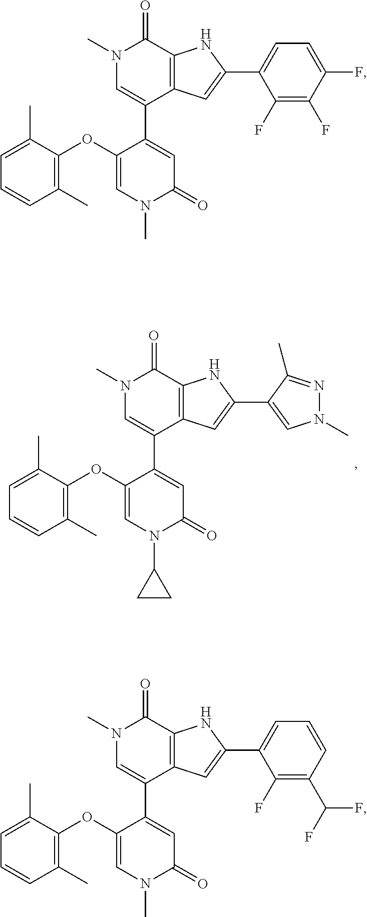 [SMILES: Cc1cccc(C)c1Oc1cn(C)c(=O)cc1-c1cn(C)c(=O)c2[nH]c(-c3ccc(F)c(F)c3F)cc12 ; Cc1cccc(C)c1Oc1cn(C2CC2)c(=O)cc1-c1cn(C)c(=O)c2[nH]c(-c3cn(C)nc3C)cc12 ; Cc1cccc(C)c1Oc1cn(C)c(=O)cc1-c1cn(C)c(=O)c2[nH]c(-c3cccc(C(F)F)c3F)cc12]

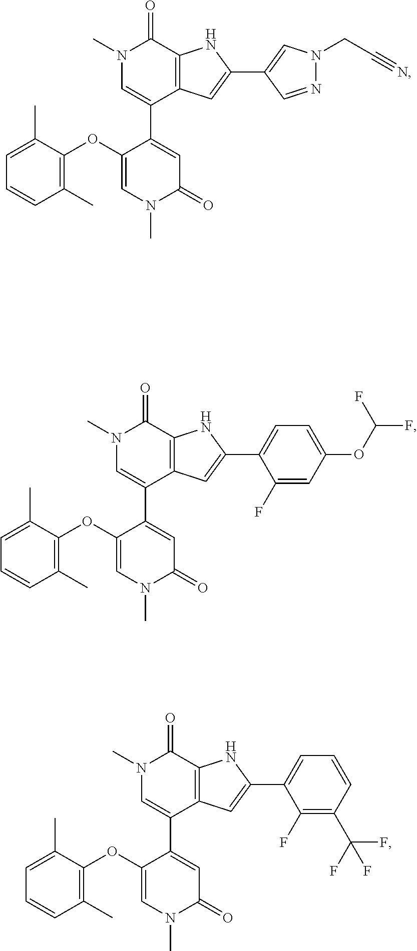 [SMILES: Cc1cccc(C)c1Oc1cn(C)c(=O)cc1-c1cn(C)c(=O)c2[nH]c(-c3cnn(CC#N)c3)cc12 ; Cc1cccc(C)c1Oc1cn(C)c(=O)cc1-c1cn(C)c(=O)c2[nH]c(-c3ccc(OC(F)F)cc3F)cc12 ; Cc1cccc(C)c1Oc1cn(C)c(=O)cc1-c1cn(C)c(=O)c2[nH]c(-c3cccc(C(F)(F)F)c3F)cc12]

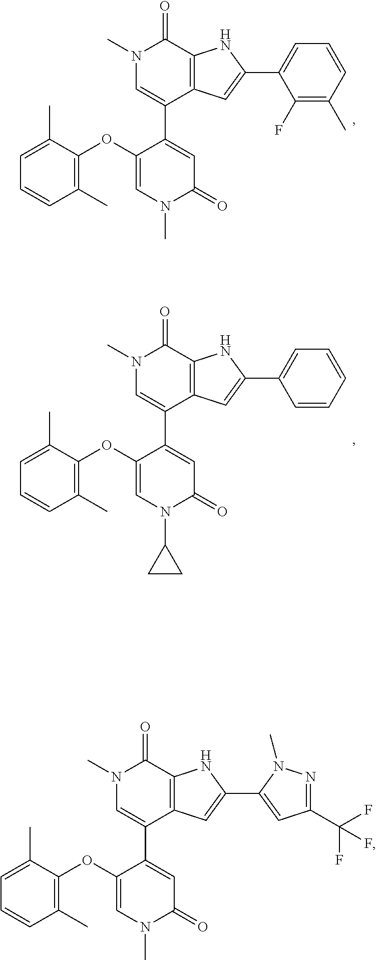 [SMILES: Cc1cccc(-c2cc3c(-c4cc(=O)n(C)cc4Oc4c(C)cccc4C)cn(C)c(=O)c3[nH]2)c1F ; Cc1cccc(C)c1Oc1cn(C2CC2)c(=O)cc1-c1cn(C)c(=O)c2[nH]c(-c3ccccc3)cc12 ; Cc1cccc(C)c1Oc1cn(C)c(=O)cc1-c1cn(C)c(=O)c2[nH]c(-c3cc(C(F)(F)F)nn3C)cc12]

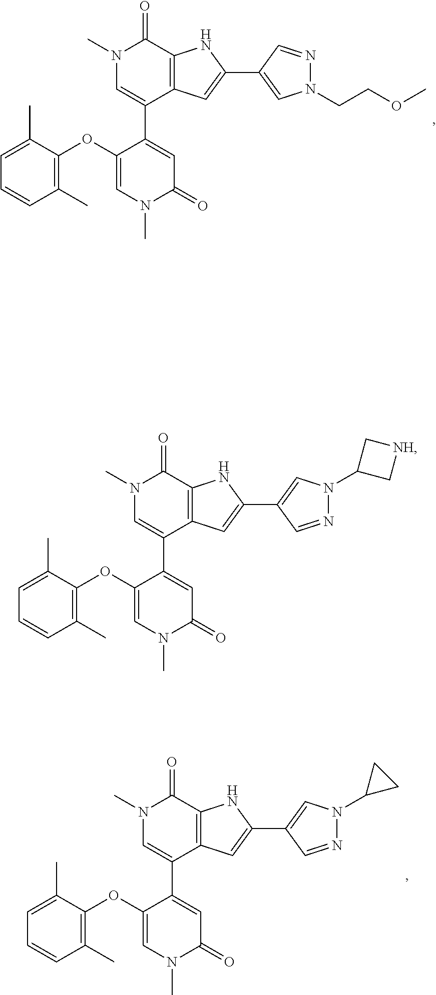 [SMILES: COCCn1cc(-c2cc3c(-c4cc(=O)n(C)cc4Oc4c(C)cccc4C)cn(C)c(=O)c3[nH]2)cn1 ; Cc1cccc(C)c1Oc1cn(C)c(=O)cc1-c1cn(C)c(=O)c2[nH]c(-c3cnn(C4CNC4)c3)cc12 ; Cc1cccc(C)c1Oc1cn(C)c(=O)cc1-c1cn(C)c(=O)c2[nH]c(-c3cnn(C4CC4)c3)cc12]

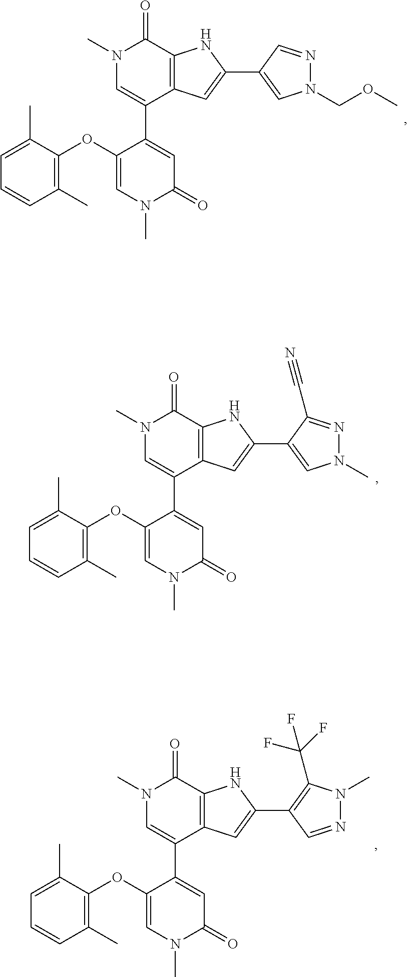 [SMILES: COCn1cc(-c2cc3c(-c4cc(=O)n(C)cc4Oc4c(C)cccc4C)cn(C)c(=O)c3[nH]2)cn1 ; Cc1cccc(C)c1Oc1cn(C)c(=O)cc1-c1cn(C)c(=O)c2[nH]c(-c3cn(C)nc3C#N)cc12 ; Cc1cccc(C)c1Oc1cn(C)c(=O)cc1-c1cn(C)c(=O)c2[nH]c(-c3cnn(C)c3C(F)(F)F)cc12]

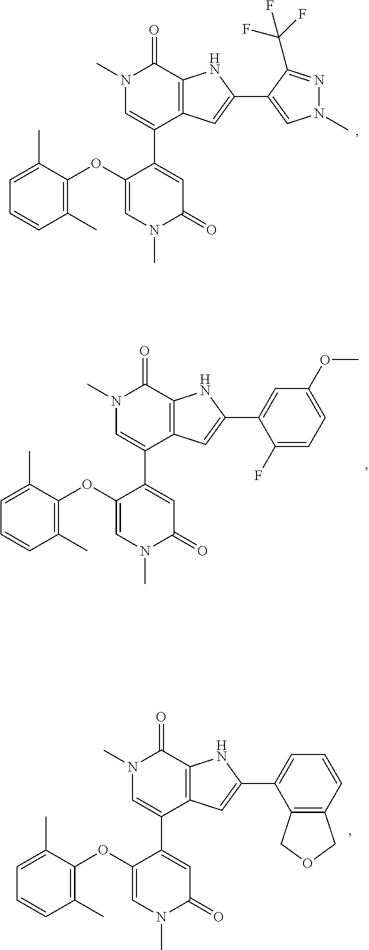 [SMILES: Cc1cccc(C)c1Oc1cn(C)c(=O)cc1-c1cn(C)c(=O)c2[nH]c(-c3cn(C)nc3C(F)(F)F)cc12 ; COc1ccc(F)c(-c2cc3c(-c4cc(=O)n(C)cc4Oc4c(C)cccc4C)cn(C)c(=O)c3[nH]2)c1 ; Cc1cccc(C)c1Oc1cn(C)c(=O)cc1-c1cn(C)c(=O)c2[nH]c(-c3cccc4c3COC4)cc12]

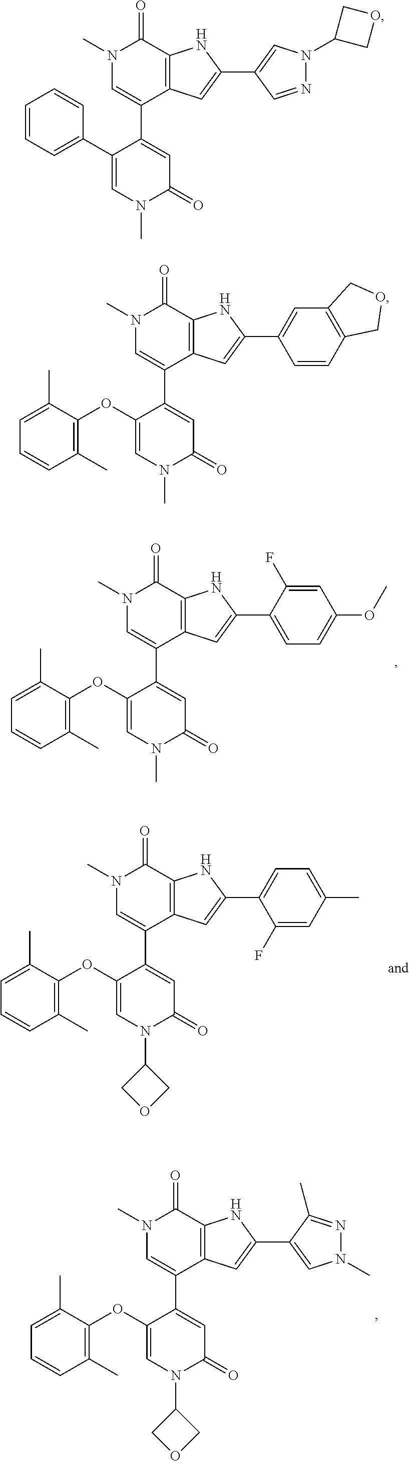

or a pharmaceutically acceptable salt thereof, or N-oxide thereof.

In some embodiments, the compound of the formulae disclosed herein is a tautomer, a stereoisomer or a mixture of stereoisomers, a pharmaceutically acceptable salt, a hydrate, a deuterated derivative, or an N-oxide thereof.

In some embodiments, the present disclosure provides a pharmaceutical composition comprising a compound defined in this disclosure and one or more pharmaceutically acceptable excipients. In some embodiments the disclosure refers to “a disease” or “a disorder”. In all instances, reference to “a disease” or “a disorder” should be understood to mean “a disease” and/or “a disorder”.

In some embodiments, the present disclosure provides a compound or a pharmaceutical composition as defined in this disclosure, for use as a medicament.

In some embodiments, the present disclosure provides the use of a compound or a pharmaceutical composition as defined in this disclosure, for the manufacture of a medicament.

In some embodiments, the medicament has a therapeutic effect in the treatment of an immuno or autoimmune disease, e.g., arthritis, rheumatoid arthritis, psoriasis/psoriatic arthritis, and/or an inflammatory disease, e.g., inflammatory skin disorders, respiratory diseases, gastrointestinal diseases, eye diseases, cancers, rheumatic diseases, demyelinating diseases, fibrotic diseases, and/or a myeloproliferative neoplastic disorder.

In some embodiments, the present disclosure provides a compound disclosed herein, or a pharmaceutical composition comprising the compound, for use in a method of treatment of an immuno or autoimmune disease, e.g., arthritis, rheumatoid arthritis, psoriasis/psoriatic arthritis, and/or an inflammatory disease, e.g., inflammatory skin disorders, respiratory diseases, gastrointestinal diseases, eye diseases, cancers, rheumatic diseases, demyelinating diseases, fibrotic diseases, and/or a myeloproliferative neoplastic disorder.

In some embodiments, the present disclosure provides a method for the treatment of an immuno or autoimmune disease, e.g., arthritis, rheumatoid arthritis, psoriasis/psoriatic arthritis, and/or an inflammatory disease, e.g., inflammatory skin disorders, respiratory diseases, gastrointestinal diseases, eye diseases, cancers, rheumatic diseases, demyelinating diseases, fibrotic diseases, and/or a myeloproliferative neoplastic disorder, said method comprising administering to a subject, an effective amount of the compound as defined in any of the embodiments disclosed herein, or a pharmaceutical composition as defined in any of the embodiments disclosed herein.

In some embodiments, the present disclosure provides the use of a compound, or use of a pharmaceutical composition comprising the compound, for the manufacture of a medicament for the treatment of an inflammatory disease, e.g., skin diseases, rheumatic diseases and fibrotic diseases, or an immuno or autoimmune disease, e.g., arthritis, rheumatoid arthritis, psoriasis/psoriatic arthritis, said method comprising administering to a subject, an effective amount of the compound as defined in any of the embodiments disclosed herein, or a pharmaceutical composition as defined in any of the embodiments disclosed herein.

Selective BET BDII inhibitors, such as the compounds disclosed herein, may in one or more embodiments, be of value and used in the treatment of the inflammatory disorders, immune disorders, autoimmune disorders, which include diseases that have or may have an inflammatory or autoimmune component, including the following non-limiting examples of disorders and diseases.

In some embodiments, the present disclosure provides a compound or a pharmaceutical composition as defined in this disclosure, for use in a method of treatment or prophylaxis of an inflammatory disease, e.g., inflammatory skin disorders, respiratory diseases, gastrointestinal diseases, eye diseases, cancers, rheumatic diseases, demyelinating diseases and fibrotic diseases and or an immuno or autoimmune disease, e.g., arthritis, rheumatoid arthritis, psoriasis/psoriatic arthritis, multiple sclerosis, lupus, systemic lupus erythematosus, inflammatory bowel disease, Addison's disease, Graves' disease, Sjögren's syndrome, thyroiditis, myasthenia gravis, Guillain-Barre syndrome, chronic inflammatory demyelinating polyneuropathy, scleroderma and autoimmune vasculitis, and or a myeloproliferative neoplastic disorder, e.g., chronic myelogenous leukemia, polycythemia vera, primary myelofibrosis (also called chronic idiopathic myelofibrosis), essential thrombocythemia, chronic neutrophilic leukemia, chronic eosinophilic leukemia, and acute leukemia. Immuno-inflammatory indications include rheumatoid arthritis, systemic lupus erythematosus, and multiple sclerosis.

In some embodiments, the present disclosure provides a compound or a pharmaceutical composition as defined in this disclosure, for use in a method of treatment of an inflammatory disease, e.g., inflammatory skin disorders, respiratory diseases, gastrointestinal diseases, eye diseases, cancers, rheumatic diseases, demyelinating diseases and fibrotic diseases and or an immuno or autoimmune disease, e.g., arthritis, rheumatoid arthritis, psoriasis/psoriatic arthritis, multiple sclerosis, lupus, systemic lupus erythematosus, inflammatory bowel disease, Addison's disease, Graves' disease, Sjögren's syndrome, thyroiditis, myasthenia gravis, Guillain-Barre syndrome, chronic inflammatory demyelinating polyneuropathy, scleroderma and autoimmune vasculitis, and or a myeloproliferative neoplastic disorder, e.g., chronic myelogenous leukemia, polycythemia vera, primary myelofibrosis (also called chronic idiopathic myelofibrosis), essential thrombocythemia, chronic neutrophilic leukemia, chronic eosinophilic leukemia, and acute leukemia. Immuno-inflammatory indications include rheumatoid arthritis, systemic lupus erythematosus, and multiple sclerosis.

In some embodiments, the present disclosure provides a method for the treatment or prophylaxis of an inflammatory disease, e.g., inflammatory skin disorders, respiratory diseases, gastrointestinal diseases, eye diseases, cancers, rheumatic diseases, demyelinating diseases, and fibrotic diseases, and or an immuno or autoimmune disease, e.g., arthritis, rheumatoid arthritis, psoriasis/psoriatic arthritis, multiple sclerosis, lupus, systemic lupus erythematosus, inflammatory bowel disease, Addison's disease, Graves' disease, Sjögren's syndrome, thyroiditis, myasthenia gravis, Guillain-Barre syndrome, chronic inflammatory demyelinating polyneuropathy, scleroderma and autoimmune vasculitis, and or a myeloproliferative neoplastic disorder, e.g., chronic myelogenous leukemia, polycythemia vera, primary myelofibrosis (also called chronic idiopathic myelofibrosis), essential thrombocythemia, chronic neutrophilic leukemia, chronic eosinophilic leukemia, and acute leukemia. Immuno-inflammatory indications include rheumatoid arthritis, systemic lupus erythematosus, and multiple sclerosis, said method comprising administering to a subject, an effective amount of a compound or a pharmaceutical composition as defined in this disclosure.

In some embodiments, the present disclosure provides a method for the treatment of an inflammatory disease, e.g., inflammatory skin disorders, respiratory diseases, gastrointestinal diseases, eye diseases, cancers, rheumatic diseases, demyelinating diseases, and fibrotic diseases, and or an immuno or autoimmune disease, e.g., arthritis, rheumatoid arthritis, psoriasis/psoriatic arthritis, multiple sclerosis, lupus, systemic lupus erythematosus, inflammatory bowel disease, Addison's disease, Graves' disease, Sjögren's syndrome, thyroiditis, myasthenia gravis, Guillain-Barre syndrome, chronic inflammatory demyelinating polyneuropathy, scleroderma and autoimmune vasculitis, and or a myeloproliferative neoplastic disorder, e.g., chronic myelogenous leukemia, polycythemia vera, primary myelofibrosis (also called chronic idiopathic myelofibrosis), essential thrombocythemia, chronic neutrophilic leukemia, chronic eosinophilic leukemia, and acute leukemia. Immuno-inflammatory indications include rheumatoid arthritis, systemic lupus erythematosus, and multiple sclerosis. said method comprising administering to a subject, an effective amount of a compound or a pharmaceutical composition as defined in this disclosure.

In some embodiments, the present disclosure provides the use of a compound or a pharmaceutical composition as defined in this disclosure for the manufacture of a medicament for the treatment or prophylaxis of an inflammatory disease, e.g., inflammatory skin disorders, respiratory diseases, gastrointestinal diseases, eye diseases, cancers, rheumatic diseases, demyelinating diseases, and fibrotic diseases and or an immuno or autoimmune disease, e.g., arthritis, rheumatoid arthritis, psoriasis/psoriatic arthritis, multiple sclerosis, lupus, systemic lupus erythematosus, inflammatory bowel disease, Addison's disease, Graves' disease, Sjögren's syndrome, thyroiditis, myasthenia gravis, Guillain-Barre syndrome, chronic inflammatory demyelinating polyneuropathy, scleroderma and autoimmune vasculitis, and or a myeloproliferative neoplastic disorder, e.g., chronic myelogenous leukemia, polycythemia vera, primary myelofibrosis (also called chronic idiopathic myelofibrosis), essential thrombocythemia, chronic neutrophilic leukemia, chronic eosinophilic leukemia, and acute leukemia. Immuno-inflammatory indications include rheumatoid arthritis, systemic lupus erythematosus, and multiple sclerosis, said method comprising administering to a subject, an effective amount of a compound or a pharmaceutical composition as defined in this disclosure.

In some embodiments, the present disclosure provides a method of inhibiting Bromodomain and Extra-Terminal protein activity in a subject, said method comprising administering to a subject an effective amount of a compound or a pharmaceutical composition as defined in this disclosure.

In some embodiments, the present disclosure provides a compound, or a pharmaceutical composition comprising the compound, for use in a method of inhibiting Bromodomain and Extra-Terminal protein activity in a subject, said method comprising administering to a subject an effective amount of a compound or a pharmaceutical composition as defined in this disclosure.

In some embodiments, the present disclosure use of a compound, or a pharmaceutical composition comprising the compound, for use in the manufacture of a medicament for the inhibition of Bromodomain and Extra-Terminal protein activity in a subject, said method comprising administering to a subject an effective amount of a compound or a pharmaceutical composition as defined in this disclosure.

In some embodiments, the present disclosure provides a method of treating a disorder associated with Bromodomain and Extra-Terminal protein activity in a subject, said method comprising administering to a subject an effective amount of a compound or a pharmaceutical composition as defined in this disclosure.

In some embodiments, the present disclosure provides a compound, or a pharmaceutical composition comprising the compound, for use in a method of treating a disorder associated with Bromodomain and Extra-Terminal protein activity in a subject, said method comprising administering to a subject an effective amount of a compound or a pharmaceutical composition as defined in this disclosure.

In some embodiments, the present disclosure provides a compound, or a pharmaceutical composition comprising the compound, for use in the manufacture of a medicament for treating a disorder associated with Bromodomain and Extra-Terminal protein activity in a subject, said method comprising administering to a subject an effective amount of a compound or a pharmaceutical composition as defined in this disclosure.

Selective BET BDII inhibitors, such as the compounds disclosed herein, may, in one or more embodiments, be of value and used in the treatment or amelioration of the following non-limiting examples of disorders and diseases.

Selective BET BDII inhibitors, such as the compounds disclosed herein, may, in one or more embodiments, be of value and used in the treatment or amelioration of inflammatory disorders, immune disorders, and autoimmune disorders, which include diseases that have or may have an inflammatory or autoimmune component.

Selective BET BDII inhibitors, such as the compounds disclosed herein, may, in one or more embodiments, be of value and used in the treatment or amelioration of myeloproliferative neoplastic disorders, which may include diseases that have or may have an inflammatory or autoimmune component.

The inflammatory disorder, immune disorder, or autoimmune disorder may be a skin disorder selected from acne, inflammatory acne, acne fulminans, angiofibroma, nodular papulopustular acne, acne conglobata, acute erysipelas, alopecia, alopecia areata, alopecia totalis, atopic dermatitis, alopecia universalis, autoimmune bullous skin disorder such as pemphigus vulgaris (PV) or bullous pemphigoid (BP), bacterial skin infections, viral skin infections, bullous diseases, cellulitis, cutaneous abscesses, carbuncles, chronic hand eczema, cutaneous mastocytosis, Dercum disease, dermatological pain, dermatological inflammation, contact dermatitis, dermatitis, dermatitis herpetiformis, dermatomyositis, chronic atypical neutrophilic dermatosis with lipodystrophy and elevated temperature (CANDLE), neutrophilic dermatoses, such as pyoderma gangrenosum and Sweets syndrome, paronychial infections, pustulosis palmoplantaris edematous, erythema multiforme, erythema nodosum, granuloma annulare, pemphigus, epidermal necrolysis pemphigus, paraneoplastic pemphigus, erythrasma, ecthyma, eczema, folliculitis, furuncles, gustatory sweating, hyperhidrosis, Hailey-Hailey disease, hives, hidradenitis suppurativa, hypertrophic scars, impetigo, ichthyosis, ischemic necrosis, keloids, necrotizing subcutaneous infections, actinic keratosis, keratosis pilaris, miliaria, molluscum contagiosum, lichen planus, netherton syndrome, pityriasis rubra pilaris, psoriasis, pruritus, prurigo nodularis, rashes, rosacea, pediculosis, pityriasis rosea, scleroderma, scalded skin syndrome, skin rash, skin irritation, skin sensitization (e.g., contact dermatitis or allergic contact dermatitis), trauma or injury to the skin, post-operative or post-surgical skin conditions, wounds, burns (including chemical, electrical fire, friction, radiation, temperature related, thermal and cold), sunburn, scarring, scabies, skin ulcers, urticaria pigmentosa, urticarial and chronic idiopathic pruritus, vitiligo, warts, and xerosis.

The inflammatory disorder, immune disorder, or autoimmune disorder may be a respiratory disease selected from asthma, bronchiectasis, bronchiolitis, byssinosis, chronic obstructive pulmonary disease (COPD), cystic fibrosis, hypersensitivity pneumonitis, mesothelioma, pneumoconiosis, (idiopathic) pulmonary fibrosis, rhinitis, rhinosinusitis, and sarcoidosis.

The inflammatory disorder, immune disorder, or autoimmune disorder may be a gastrointestinal disease selected from celiac disease, eosinophilic esophagitis, inflammatory bowel disease, and retroperitoneal fibrosis.

The inflammatory disorder, immune disorder, or autoimmune disorder may be an eye disease selected from conjunctivitis, dry eye syndrome, iritis, keratitis, macular degeneration, myasthenia gravis, scleritis, Sjögran's syndrome, and uveitis.

The inflammatory disorder, immune disorder, or autoimmune disorder may be a cardiovascular disease or associated disorder, selected from cerebrovascular disease, aorta disease, arrhythmias, atherosclerosis, aneurysm, angina, stroke, carditis, cardiac hypertrophy, cardiomyopathy, endocarditis, coronary artery disease, deep vein thrombosis, heart attack, heart disease, heart failure, Marfan syndrome, myocarditis, peripheral artery disease, pericarditis. pulmonary embolism, rheumatic heart disease, thrombosis, valvular heart disease, ventricular heart disease, ventricle dysfunction, and vascular diseases.

The inflammatory disorder, immune disorder, or autoimmune disorder may be a systemic indication selected from Addison's disease, AIDS, ankylosing spondylitis, atherosclerosis, arthritis, Behcet's disease, cryopyrin-associated periodic syndromes (CAPS), chronic kidney diseases (including, but not limited to nephritis, nephropathy, hypertensive nephropathy, HIV-associated nephropathy, IgA nephropathy, familial Mediterranean fever, focal segmental glomerulosclerosis, Grave's disease, juvenile arthritis, lymphangitis, lymphadenitis, lupus nephritis, minimal change disease, neurofibromatoses, polycystic kidney disease and tubular interstitial nephritis), acute kidney injury disease or condition (including, but are not limited to ischemia-reperfusion induced, cardiac and major surgery induced, percutaneous coronary intervention induced, radio-contrast agent induced, sepsis induced, pneumonia induced, and drug toxicity induced), giant cell arthritis, glomerulonephritis, gout, hepatitis, hepatitis B, hepatitis C, hypophysitis, Kawasaki disease, liver fibrosis, multiple sclerosis, myositis, osteoarthritis, pancreatitis, pneumonitis, polyarteritis nodosa, primary biliary cirrhosis, prostate disease, prostatitis, benign prostatic hyperplasia (BPH), psoriatic arthritis, rheumatoid arthritis, scleritis, scleroderma (cutaneous or systemic), sclerosing cholangitis, sepsis, systemic lupus erythematosus, systemic mastocytosis, Takayasu's arthritis, thyroiditis, toxic shock, vasculitis, warm autoimmune hemolytic anemia, and Wegener's granulomatosis.

The inflammatory disorder, immune disorder, or autoimmune disorder may be an autoimmune disease or indication where immunosuppression would be desirable, for instance, to avoid organ transplant rejection and graft versus host disease (chronic or acute).

Selective BET BDII inhibitors, such as the compounds disclosed herein, may in one or more embodiments, be of value and used in the treatment or amelioration of cancers.

The cancer may be a skin or systemic cancer, selected from acoustic neuroma, anal cancer, bladder cancer, Bowen's disease, brain cancer, breast cancer, carcinomas including basal cell carcinoma, bile duct carcinoma, bronchogenic carcinoma, choriocarcinoma, embryonal carcinoma, cystadenocarcinoma, epithelial carcinoma, medullary carcinoma, NUT midline carcinoma (NMC), papillary carcinoma, papillary adenocarcinomas, renal cell carcinoma, sebaceous gland carcinoma, small cell lung carcinoma, squamous cell carcinoma, and sweat gland carcinoma, cervical cancer, chordoma, colon cancer, colorectal cancer, craniopharyngioma, dysproliferative changes (dysplasias and metaplasias), endometrial cancer, ependymoma, esophageal cancer, essential thrombocythemia, estrogen-receptor positive breast cancer, Ewing's tumour, genital cancer, cancer of the cervix, cancer of the vulva, vulvar intraepithelial neoplasia (VIN), cancer of the vagina, germ cell testicular cancer, gastrointestinal cancers, gastric cancer, glioblastoma, glioma, heavy chain disease, hemangioblastoma, hepatocellular cancer, hepatoma, hormone insensitive prostate cancer, keratinocyte carcinomas, kidney cancer, leukaemias including acute leukaemia, acute lymphocytic leukaemia, acute myeloid leukaemia, acute myelocytic leukaemia (monocytic, myeloblastic, adenocarcinoma, angiosarcoma, astrocytoma, myelomonocytic and promyelocytic), acute t-cell leukaemia, chronic leukaemia, chronic lymphocytic leukaemia, chronic myelocytic (granulocytic) leukaemia, chronic myelogenous leukaemia, erythroleukemia, lymphoblastic leukaemia, and myelogenous leukaemia, liver cancer, lung cancer, lymphoid malignancies of T-cell or B-cell origin, lymphomas (Hodgkin's and non-Hodgkin's) including cutaneous T-cell lymphoma, diffuse large B-cell lymphoma, and follicular lymphoma, cutaneous (skin) lymphomas, malignancies and hyperproliferative disorders including of the bladder, breast, colon, lung, ovaries, pancreas, prostate, skin and uterus, advanced malignancies, medulloblastoma, melanoma, meningioma, Merkel cell cancer mesothelioma, metastatic cancer, multiple myeloma, myeloma, pancreatic cancer, myelofibrosis, myeloproliferative neoplasms, neuroblastoma, non-small cell lung cancer, head and neck cancer, oligodendroglioma, oral cancer, ovarian cancer, pancreatic cancer, pinealoma, polycythemia vera, prostate cancer, rectal cancer, retinoblastoma, sarcomas including chondrosarcoma, endotheliosarcoma, fibrosarcoma, gliosarcoma, leiomyosarcoma, liposarcoma, lymphagioendotheliosarcoma, lymphangiosarcoma, myxosarcoma, Castleman's disease and Kaposi's sarcoma, osteogenic sarcoma, and rhabdomyosarcoma, seminoma, skin cancer, skin adnexal tumors, and sarcomas, small cell lung cancer, solid tumors, stomach cancer, synovioma, testicular tumours, thyroid cancer, uterine cancer, Waldenstrom's macroglobulinemia, and Wilms' tumour.

The myeloproliferative neoplastic disorder may be a chronic myelogenous leukemia, polycythemia vera, primary myelofibrosis (also called chronic idiopathic myelofibrosis), essential thrombocythemia, chronic neutrophilic leukemia, chronic eosinophilic leukemia, and may develop into or be an acute leukemia.

Selective BET BDII inhibitors, such as the compounds disclosed herein, may, in one or more embodiments, be used to provide male contraception.

Selective BET BDII inhibitors, such as the compounds disclosed herein, may, in one or more embodiments, be of use in the treatment or amelioration of obesity, dyslipidaemia, cholesteatoma, hypercholesterolemia, Alzheimer's disease, metabolic syndrome, hepatic steatosis, type I diabetes, type II diabetes, and complications from diabetes, insulin resistance, and diabetic retinopathy or diabetic neuropathy.

Selective BET BDII inhibitors, such as the compounds disclosed herein, may, in one or more embodiments, be of use in the treatment or amelioration of an immune system dysfunction, a viral disease, a bacterial disease, a yeast disease, non-inflammatory acne, an allergic disease, asthma, food allergy, rhinitis, an IL-6 pathway-related disease, an immune response, and a hyperproliferative disorder.

Selective BET BDII inhibitors, such as the compounds disclosed herein, may, in one or more embodiments, be of use in the treatment or amelioration of Aicardi-Gouti\tilde{e}res syndrome, chilblain lupus, stimulator of interferon genes-Associated Vasculopathy with onset in Infancy (SAVI), Singleton-Merten syndrome, retinal vasculopathy with cerebral leukodystrophy, autoimmune uveitis, lupus, systemic sclerosis, an autoimmune thyroid disease, an allograft rejection, a graft-versus-host disease, an allograft rejection reaction, and a graft-versus-host reaction.

Selective BET BDII inhibitors, such as the compounds disclosed herein, may, in one or more embodiments, be of use in the treatment or amelioration of disorders caused by a virus, such as Epstein-Barr virus (EBV), HIV, HTLV 1, herpes simplex virus (HSV), varicella zoster virus (VZV), and human papillomavirus (HPV).

Selective BET BDII inhibitors, such as the compounds disclosed herein, may, in one or more embodiments, be of use in the treatment or amelioration of mucopurulent cervicitis (MPC), urethritis, nongonococcal urethritis (NGU), vulvar disorders, vulvodynia, vulvar pain, vulvar dystrophy, pelvic inflammation, endometritis, salpingitis, oophoritis, dyspareunia, anal and rectal disease, anal abscess/fistula, anal fissure, anal warts, hemorrhoids, anal itch, pruritus ani, fecal incontinence, constipation, and polyps of the colon and rectum.

Selective BET BDII inhibitors, such as the compounds disclosed herein, may, in one or more embodiments, be of use in the restoration of integrity or acceleration of the restoration of the integrity of an area of broken or damaged tissue, skin or mucosa, and in the reduction and amelioration of scar formation or scars.

Selective BET BDII inhibitors, such as the compounds disclosed herein, may, in one or more embodiments, be of use in the treatment or amelioration of pyoderma gangrenosum (PG), palmar plantar pustulosis (PPP), and generalized pustular psoriasis (GPP).

Selective BET BDII inhibitors, such as the compounds disclosed herein, may, in one or more embodiments, be of use in the treatment or amelioration of multiple sclerosis, rheumatoid arthritis, and rhinosinusitis.

Selective BET BDII inhibitors, such as the compounds disclosed herein, may, in one or more embodiments, be of use in the treatment or amelioration of cryopyrin-associated periodic syndromes (CAPS), cardiovascular disease, cerebrovascular disease, familial Mediterranean fever, Grave's disease, liver fibrosis, neurofibromatoses, myocarditis, pericarditis, prostate disease, prostatitis, benign prostatic hyperplasia (BPH), systemic mastocytosis, and warm autoimmune hemolytic anemia.

Selective BET BDII inhibitors, such as the compounds disclosed herein, may, in one or more embodiments, be of use in the treatment or amelioration of angiofibroma, chronic hand eczema, cutaneous mastocytosis, urticaria pigmentosa, neutrophilic dermatoses such as pyoderma gangrenosum and Sweets syndrome, chronic atypical neutrophilic dermatosis with lipodystrophy and elevated temperature (CANDLE), ichthyosis, keloids, scars, hypertrophic scars, netherton syndrome, pruritus, prurigo nodularis, and urticaria pigmentosa.

Selective BET BDII inhibitors, such as the compounds disclosed herein, may, in one or more embodiments, also be of value and used in the palliation, diagnosis or prevention of any disease, disorder or condition in humans of one or more of the aforesaid non-limiting examples of disorders and diseases.

The present disclosure provides specific BET inhibitors (e.g., Compound A) that have been found to be surprisingly effective against an inflammatory autoimmune skin disorder psoriasis and can provide an effective treatment against other skin diseases and disorders e.g., skin diseases and disorders having an inflammatory and/or autoimmune component. In some embodiments, the present disclosure provides potent and selective BET inhibitors (e.g., compounds of the formulae disclosed herein) that can provide a new and effective treatment and relief for skin disorders or diseases, such as psoriasis, pyoderma gangrenosum (PG), palmoplantar pustulosis (PP), psoriasis, generalized pustular psoriasis (GPP), or other skin or skin-related diseases and disorders.

The present disclosure provides specific BET inhibitors (e.g., Compound A) that have been found to be surprisingly effective against arthritis, a joint or joint related disorders or diseases. In some embodiments, the present disclosure provides potent and selective BET inhibitors (e.g., compounds of the formulae disclosed herein) that can provide a new and effective treatment and relief for joint disorders or diseases, such as arthritis, bursitis, Ehlers-Danlos syndrome, epicondylitis, Felty syndrome, gouty arthritis, psoriatic arthritis, osteoarthritis, rheumatoid arthritis, Still's disease, tenosynovitis, synovitis, Sjögren's Syndrome, Lyme disease, Whipple disease, bone cancer, lupus or other autoimmune joint disorders. In some embodiments, the disease is rheumatoid arthritis.

The present disclosure provides selective BET inhibitors (e.g., compounds of the formulae disclosed herein) that can provide new and effective treatment and relief for joint related diseases and disorders. Joints may be infected by many types of microorganisms (bacteria, fungi, viruses) and occasionally by animal parasites. Infection related joint diseases and disorders include infection by direct contamination, by way of the bloodstream e.g., through the synovial blood vessels, and by extension from adjacent bony infections (osteomyelitis). Infectious arthritis may affect one joint (monarthritis) or a few joints (oligoarthritis) rather than many (polyarthritis). Joints or parts thereof can be damaged e.g., cartilage by for example through staphylococci, hemolytic streptococci, and pneumococci infections, e.g., bone through tuberculosis such as tuberculous spondylitis (Pott disease), or through Coccidioides immitis, brucellosis, such as brucella suis, leprosy (Hansen disease), rubella (German measles) and serum hepatitis, viral synovitis, dranunculiasis (Guinea worm disease), sexually transmitted diseases, including gonorrhea, reactive arthritis (Reiter disease), congenital syphilis such as Clutton joint lesion, and Yaws, which leads to skeletal lesions. Inflammation may destroy the joint cartilage and underlying bone and cause irreparable deformities. Adhesions between the articulating members are frequent in such cases, and the resulting fusion with loss of mobility is called ankylosis such as ankylosing spondylitis, (Marie-Strompell disease or Bechterew disease). Another type of arthritis is associated with chronic intestinal diseases, regional enteritis, inflammatory bowel disease, cirrhosis, and Whipple disease.

In addition to joint disorders and diseases resulting from any of the above, the present disclosure provides potent and selective BET inhibitors (e.g., compounds of the formulae disclosed herein) that may also provide new and effective treatment or relief for noninflammatory joint diseases, injury and degenerative disorders. Trauma to joints includes blunt injuries, mild sprains, fractures and dislocations, ligamentous, tendinous, and capsular tears, tears in the semilunar cartilages (menisci), and hemarthrosis. Degenerative joint disease includes osteoarthritis, arthrosis deformans, precocious osteoarthritis congenital dysplasia malum coxae senilis, spondylosis, chondromalacia patellae, metabolic diseases such gouty arthritis, podagra, ochronotic arthropathy, chondrocalcinosis, or pseudogout, mucopolysaccharidoses, Hurler syndrome, Morquio disease, and polyepiphyseal dysplasias.

The present disclosure provides potent and selective BET inhibitors (e.g., compounds of the formulae disclosed herein) that may also provide new and effective treatment or relief for secondary joint diseases and disorders, including hemorrhagic joints, hemarthrosis, villonodular synovitis, joint diseases that arise in association with aseptic necrosis e.g., can occur with fractures, osteochondritis dissecans, slipped epiphysis, Osgood-Schlatter, Legg-Calvé-Perthes, endocrine-malfunctioning resultant joint disorders, acromegaly, neurogenic arthropathy, Charcot joint, hypertrophic osteoarthropathy, reflex sympathetic dystrophy, joint tumors, synovial chondromatosis, cartilaginous nodules, synovial osteochondromatosism, synoviomas, synovial sarcomas, and polymyalgia rheumatica.

The present disclosure provides selective BET inhibitors (e.g., compounds of the formulae disclosed herein) that can provide new and effective treatment and relief for a fibrosis or fibrosis-associated condition. The present disclosure provides specific BET inhibitors (e.g., Compound A, Compound B, Compound C, Compound D) that can retard the progression or severity of indicators of fibrosis e.g., pulmonary, renal, or kidney fibrosis.

The methods and compositions of the present disclosure can in some embodiments be useful therapeutically for a fibrosis or fibrosis-associated conditions affecting any tissue including, for example, fibrosis of an internal organ, a cutaneous or dermal fibrosing disorder, and fibrotic conditions of the eye. In some embodiments, the fibrosis or fibrosis-associated conditions include fibrosis of internal organs (e.g., liver, lung, kidney, heart blood vessels, gastrointestinal tract). In some embodiments, the fibrosis or fibrosis-associated conditions include pulmonary fibrosis, idiopathic fibrosis, autoimmune fibrosis, myelofibrosis, liver cirrhosis, veno-occlusive disease, mesangial proliferative glomerulonephritis, crescentic glomerulonephritis, diabetic nephropathy, renal interstitial fibrosis, renal fibrosis in subjects receiving cyclosporin, allograft rejection, HIV associated nephropathy. In some embodiments, the fibrosis-associated disorders include systemic sclerosis, eosinophilia-myalgia syndrome, and fibrosis-associated CNS disorders such as intraocular fibrosis. In some embodiments, dermal fibrosis disorders include, for example, scleroderma, morphea, keloids, hypertrophic scars, familial cutaneous collagenoma, and connective tissue nevi of the collagen type. In some embodiments, fibrotic conditions of the eye include conditions such as diabetic retinopathy, post-surgical scarring (for example, after glaucoma filtering surgery and after crossed eyes (strabismus) surgery), and proliferative vitreoretinopathy. In some embodiments, fibrotic conditions that may be treated by the compounds, compositions, and methods disclosed herein may result, for example, from rheumatoid arthritis, diseases associated with prolonged joint pain and deteriorated joints, progressive systemic sclerosis, polymyositis, dermatomyositis, eosinophilic fasciitis, morphea, Raynaud's syndrome, and nasal polyposis.

Organ disease often leads to organ fibrosis and which, in turn, can lead to death. Fibrosis may follow a path independent of the organ. Fibrosis may be the result of excessive wound healing. In the kidney, this results mainly in glomerulosclerosis, tubular atrophy and dilation, tubulointerstitial fibrosis and capillary rarefaction. Renal fibrosis can be characterized by an excessive accumulation and deposition of extracellular matrix components. Renal fibrosis is not a simple, uniform scarring, but a dynamic process involving many, if not all, renal and infiltrating cell types. Kidneys often fail to repair themselves completely. Kidney cells can facilitate and increase the secretion of pro-fibrosis factors. When a normal healing response fails, scarring continues, and this can cause chronic kidney disease (CKD). Progressive scarring replaces normal kidney tissue with fibrotic tissue and kidney function is lost, which may lead to kidney failure. MMP-2, MCP-1 and TGF-β have been shown to identify patients with fibrosis and future poor renal outcomes.

The present disclosure provides specific BET inhibitors (e.g., Compound A) that have been found to be surprisingly effective against renal fibrosis and renal fibrosis-related conditions and/or may provide a suitable treatment in limiting or slowing its progression. In some embodiments, the present disclosure provides potent and selective BET inhibitors (e.g., compounds of the formulae disclosed herein) that can provide a new and effective treatment and relief for fibrosis and fibrosis-related conditions, e.g., renal fibrosis and renal fibrosis-related conditions and/or limit or slow its progression. The present disclosure provides potent and selective BET inhibitors (e.g., compounds of the formulae described herein) that can provide new and effective treatment or relief for inflammatory fibrosis (e.g., renal fibrosis) and/or limit or slow its progression, e.g., when administered orally.

The present disclosure provides potent and selective BET inhibitors (e.g., compounds of the formulae disclosed herein) that may also provide new and effective treatment or relief for noninflammatory fibrosis (e.g., renal fibrosis) diseases, injury, and degenerative disorders and/or limit or slow their progression, e.g., when administered orally.

The present disclosure further provides specific BET inhibitors (e.g., Compound A) that have been found to be surprisingly effective against PF and PF-related conditions and/or may provide a suitable treatment in limiting or slowing its progression. In some embodiments, the present disclosure provides potent and selective BET inhibitors (e.g., compounds of the formulae disclosed herein) that can provide a new and effective treatment and relief for fibrosis and fibrosis-related conditions, e.g., PF and PF-related conditions and/or limit or slow its progression. The present disclosure provides potent and selective BET inhibitors (e.g., compounds of the formulae disclosed herein) that can provide new and effective treatment or relief for inflammatory fibrosis (e.g., pulmonary fibrosis) and/or limit or slow its progression. In some embodiments, effective treatment is achieved using an oral administration of the potent and selective BET inhibitors. In some embodiments, treatment with BET inhibitors (e.g., compounds of the formulae disclosed herein) may improve lung function in a PF patient (such as an IPF patient), e.g., it may restore normal or near normal levels of lung function (e.g., at least about 80% to about 90% of normal oxygen saturation and/or at least about 40% to about 60% of normal functional lung volume) or may improve levels of lung function (e.g., improve oxygen saturation by about 5% to about 10% and/or improve functional lung volume by about 20% to about 60%, or by about 40% to about 60%. In some embodiments, treatment with BET inhibitors (e.g., compounds of the formulae disclosed herein) reduces lung fibrosis (e.g., reduction in lung fibrosis score by about 20% to about 60%). In some embodiments, treatment with BET inhibitors (e.g., compounds of the formulae disclosed herein) reduces fibrotic tissue deposition. In some embodiments, treatment with BET inhibitors (e.g., compounds of the formulae disclosed herein) reduces hydroxyproline levels in fibrotic subjects.

The present disclosure also provides potent and selective BET inhibitors (e.g., compounds of the formulae disclosed herein) that may provide new and effective treatment or relief for noninflammatory fibrosis (e.g., PF, IPF) diseases, injury, and degenerative disorders and/or limit or slow their progression, e.g., when administered orally.

BET inhibitors, such as the compounds disclosed herein, may, in one or more embodiments, be of value and used in the treatment of inflammatory disorders, immune disorders, and autoimmune disorders, which include diseases that have or may have an inflammatory or autoimmune component.

The present disclosure further provides specific BET inhibitors (e.g., Compound A) that have been found to be surprisingly effective against SLE and lupus-related conditions and/or may provide a suitable treatment in limiting or slowing its progression. In some embodiments, the present disclosure provides potent and selective BET inhibitors (e.g., compounds of the formulae disclosed herein) that can provide a new and effective treatment and relief for lupus and lupus-related conditions and/or limit or slow its progression, e.g., when administered orally. In some embodiments, treatment with BET inhibitors (e.g., compounds of the formulae disclosed herein) may improve kidney function is a lupus patient, e.g., in a SLE or CLE patient, by improving kidney function relative to an untreated patient (e.g., as evidenced by decreased proteinuria, decreased total glomerular lesions, decreased total tubular and interstitial lesions, and/or decreased total kidney lesions). In some embodiments, treatment with BET inhibitors (e.g., compounds of the formulae disclosed herein) may improve kidney function in a lupus patient (e.g., reduce total glomerular lesion score by at least about 20% to about 30%, reduce total tubular and interstitial lesion score by at least about 40 to about 50%, and/or reduce total kidney lesion score by at least about 30% to about 40%) or reduce overall kidney lesion score by about 15% to about 80% or by about 20% to about 75%. In some embodiments, administration of BET inhibitors (e.g., compounds of the formulae disclosed herein) can reduce severity of lupus, and/or suppress lupus disease in a dose dependent fashion.

The present disclosure further provides specific BET inhibitors (e.g., Compound A) that have been found to be surprisingly effective against CNS diseases including autoimmune diseases that attack the central nervous system (CNS), CNS inflammation, and demyelinating diseases, such as MS and MS-related conditions, and/or may provide a suitable treatment e.g., in limiting, slowing, and/or retarding their progression, or in reversing the course of the disease. In some embodiments, the present disclosure provides potent and selective BET inhibitors (e.g., compounds of the formulae disclosed herein) that can provide, e.g., when administered orally, a new and effective treatment and relief for MS and MS-related conditions, and/or may limit, slow, and/or retard their progression. In some embodiments, treatment with BET inhibitors (e.g., compounds of the formulae disclosed herein) may reduce MS-associated inflammation in an MS patient relative to an untreated patient, for example, evidenced by decreased levels of IFNγ and/or IL-12/IL-23p40. In some embodiments, treatment with BET inhibitors (e.g., compounds of the formulae disclosed herein) may reduce the severity of MS and MS-associated symptoms, and/or may limit, slow, and/or retard the progression of MS and MS-associated symptoms. In some embodiments, treatment with BET inhibitors (e.g., compounds of the formulae disclosed herein) may limit, slow, and/or retard the progression of demyelination, and/or its severity. In some embodiments, treatment with BET inhibitors (e.g., compounds of the formulae disclosed herein) may suppress CNS inflammation, and/or its severity. In some embodiments, treatment with BET inhibitors (e.g., compounds of the formulae disclosed herein) may limit, slow, and/or retard the progression of CNS inflammation, and/or its severity. In some embodiments, treatment with BET inhibitors (e.g., compounds of the formulae disclosed herein) may limit, slow, and/or retard the progression of autoimmune diseases that attack the CNS, and/or their severity. In some embodiments, treatment with BET inhibitors (e.g., compounds of the formulae disclosed herein) may result in a reversal (e.g., partial, or substantial) of the disease course, facilitating recovery. In some embodiments, treatment with BET inhibitors (e.g., compounds of the formulae disclosed herein) may modulate immune cells infiltrating the CNS. In some embodiments, treating with BET inhibitors (e.g., compounds of the formulae disclosed herein) may reduce the severity of axonal damage, and/or may limit, slow, and/or retard the progression of axonal damage, which can lead to weakness and/or paralysis (e.g., leg weakness and/or paralysis).

In some embodiments, the potent and selective BET inhibitors are highly selective for BDII over BDI.

In some embodiments, reference to an amount e.g., selectivity or activity etc., may reflect a mean.

It has been found that certain compounds of the disclosure may have increased activity against BRD4 BD2. In some embodiments, compounds of the disclosure have a BD2 IC50 of less than about 10 nM. In some embodiments, compounds of the disclosure may have increased selectivity for BRD4 BD2 over BRD4 BD1. In some embodiments, compounds of the disclosure have a selectivity for BRD4 BD2 over BRD4 BD1 of greater than about 1000. In some embodiments, compounds of the disclosure may have increased bioavailability. In some embodiments, compounds of the disclosure have a bioavailability following oral delivery that is sufficiently high to allow for systemic delivery of the compounds through the oral administration. In some embodiments, compounds of the disclosure have satisfactory chemical and metabolic stability. In some embodiments, compounds have a chemical half-life of more than 24 hours and a plasma half-life e.g., in humans of greater than 2 hours.

Treatment or amelioration with selective BET BDII inhibitors, such as compositions comprising the compounds disclosed herein or salts thereof (or combinations thereof), in some embodiments, may be effective if applied orally, in some other embodiments, may be effective if applied by injection, in some other embodiments, may be effective if applied topically, and in some further embodiments, may be effective if applied topically and orally or by injection and topically or by orally and injection. In one or more embodiments, treatment or amelioration with selective BET BDII inhibitors, such as compositions comprising the compounds disclosed herein or salts thereof (or combinations thereof), may be effective orally where the compounds have a reasonable e.g., >about 20% or a good bioavailability e.g., >about 25%. In some embodiments the bioavailability is >about 35%, or >about 45%, or >about 55%, or >about 65%, or >about 75%, or >about 85% thereof), may be effective orally where the compounds have good bioavailability e.g., >about 25%.

In one or more embodiments, compounds disclosed herein are active against BRD4 BD2 and selective over BRD4 BD1. In one or more embodiments, BET BDII selective protein inhibitors exhibit greater than about 100-fold selectivity, greater than about 200-fold selectivity, greater than about 250-fold selectivity, greater than about 300-fold selectivity, greater than about 350-fold selectivity greater than about 400-fold selectivity, greater than about 500-fold selectivity, greater than about 600-fold selectivity, greater than about 700-fold selectivity, greater than about 800-fold selectivity, greater than about 900-fold selectivity, greater than about 1000-fold selectivity, greater than about 2000-fold selectivity, or greater than about 5000-fold selectivity for BDII over BDI depending e.g., on the structure. In an embodiment BET BDII selective protein inhibitors exhibit greater than about 1000-fold selectivity. In one or more embodiments, BET BDII selective protein inhibitors exhibit an IC50 of <about 200 nM, <about 150 nM, <about 100 nM, <about 50 nM or <about 10 nM for BRD4 BDII. In one or more embodiments, BET BDII selective protein inhibitors exhibit an IC50 ranging from <about 200 nM to about 10 nM. In some embodiments, BET BDII selective protein inhibitors disclosed herein exhibit an IC50 ranging from about 10 nM to about 1 nM, or about 5 nM to about 0.1 nM for BRD4 BDII. In some embodiments, BET BDII selective protein inhibitors disclosed herein exhibit an IC50 ranging from about 5 nM to about 1 nM, or about 5 nM to about 0.1 nM for BRD4 BDII. In some embodiments, the IC50 is a mean value of two or more measurements.

In one or more embodiments, compounds disclosed herein are surprisingly highly active against BRD4 BD2 and surprisingly highly selective over BRD4 BD1. In one or more embodiments, certain compounds disclosed herein as BET BDI1 selective protein inhibitors surprisingly exhibit an IC50 of less than about 10 nM. In some embodiments, certain compounds exhibit an IC50 of less than about 8 nM, or less than about 6 nM, or less than about 5 nM, or less than about 4 nM, or less than about 3 nM, or less than about 2 nM, or about 1 nM. In one or more embodiments, certain compounds disclosed herein as BET BDII selective protein inhibitors exhibit greater than about 1000-fold selectivity. In some embodiments certain compounds exhibit greater than about 2000-fold selectivity, or greater than about 3000-fold selectivity, or greater than about 4000-fold selectivity, or greater than about 5000-fold selectivity, or greater than about 6000-fold selectivity, or greater than about 7000-fold selectivity, or greater than about 8000-fold selectivity, or greater than about 9000-fold selectivity, or greater than about 10,000-fold selectivity. In one or more embodiments, certain compounds disclosed herein as BET BDI1 selective protein inhibitors surprisingly exhibit a selectivity of greater than about 1000 and an IC50 of less than about 10 nM. In some embodiments, certain compounds disclosed herein as BET BDII selective protein inhibitors surprisingly exhibit a selectivity of greater than about 2000 and an IC50 of less than about 5 nM. In some embodiments, certain compounds disclosed herein as BET BDII selective protein inhibitors surprisingly exhibit a selectivity of greater than about 2500 and an IC50 of less than about 4 nM. In some embodiments, certain compounds disclosed herein as BET BDII selective protein inhibitors surprisingly exhibit a selectivity of greater than about 3000 and an IC50 of less than about 4 nM, or in some embodiments, a selectivity of greater than about 3000 and an IC50 of less than about 3 nM. In some embodiments, certain compounds disclosed herein as BET BDI selective protein inhibitors surprisingly exhibit a selectivity of greater than about 4000 and an IC50 of less than about 3 nM. In some embodiments, certain compounds disclosed herein as BET BDII selective protein inhibitors surprisingly exhibit a selectivity of greater than about 5000 and an IC50 of less than about 2 nM. In some embodiments, certain compounds disclosed herein as BET BDII selective protein inhibitors surprisingly exhibit a selectivity of about 1000 to about 2000 and an IC50 of less than about 10 nM. In some embodiments, certain compounds disclosed herein as BET BDII selective protein inhibitors surprisingly exhibit a selectivity of about 2000 to about 5000 and an IC50 of less than about 5 nM. In some embodiments certain compounds disclosed herein as BET BDII selective protein inhibitors surprisingly exhibit a selectivity of about 3000 to about 5000 and an IC50 of less than about 4 nM. In some embodiments, certain compounds disclosed herein as BET BDII selective protein inhibitors surprisingly exhibit a selectivity of about 4000 to about 11000 and an IC50 of less than about 3 nM. In some embodiments, certain compounds disclosed herein as BET BDIl selective protein inhibitors surprisingly exhibit a selectivity of about 4000 to about 15000 and an IC50 of less than about 3 nM. In some embodiments, certain compounds disclosed herein as BET BDII selective protein inhibitors surprisingly exhibit a selectivity of about 5000 to about 15000 and an IC50 of less than about 2.5 nM. In one or more embodiments the selectivity reflects a mean.

In addition to the compounds showing activity and selectivity other factors in selecting promising drug candidates can include for example, bioavailability, clearance, chemical stability, plasma stability, pK, and an IC50 for inflammatory biomarkers such as IL17 and IL 22 of <about 100 nM. For drug candidates for oral delivery a higher bioavailability can translate into a lower dosage and potentially fewer side effects e.g., in the alimentary canal. For drug candidates for oral delivery a higher plasma concentration over the free EC50 for BD 2 for a period of about 4 or more hours can translate into an effective drug. In one or more embodiments, the higher plasma concentration over the free EC50 for BD 2 is for a period of about 4 or more hours. In some embodiments, it is for a period of about 6 or more hours, or for a period of about 8 or more hours, or for a period of about 12 or more hours, or for a period of about 15 or more hours. In one or more embodiments, a therapeutically effective amount of drug is applied once a day. In some embodiments, it is applied two times a day e.g., where the period in which the plasma concentration is higher than the free EC50 is less than 12 hours. In some embodiments, it is applied 3 times a day.

In one or more embodiments, BET BDII selective protein inhibitors exhibit a rat microsomal stability of <about 5, <about 4, <about 3, <about 2, or <about 1 ml/min/g liver. In some embodiments, BET BDII selective protein inhibitors exhibit a rat microsomal stability of <2 about ml/min/g liver.

In some embodiments, BET BDII selective protein inhibitors with a rat microsomal stability of <about 4 ml/min/g liver or <about 3 ml/min/g are promising drug candidates, but compounds having a lower rat microsomal stability that is having a higher rate of breakdown may in some other embodiments be useful in particular contexts. In some embodiments, BET BDII selective protein inhibitors have a rat microsomal stability of <2 ml/m/g.

In one or more embodiments, BET BDII selective protein inhibitors exhibit a rat microsomal stability of >about 20 minutes, >about 40 minutes, >about 60 minutes, >about 80 minutes, >about 100 minutes, or >about 120 minutes half-life. In some embodiments, BET BDII selective protein inhibitors exhibit a rat microsomal stability of >about 60 minutes half-life. In some embodiments, BET BDII selective protein inhibitors exhibit a rat microsomal stability of >about 80 minutes half-life.

In some embodiments, BET BDII selective protein inhibitors with a rat microsomal stability of >about 20 minutes half-life are promising drug candidates, but compounds having a lower rat microsomal stability may in some other embodiments be useful in particular contexts.

In one or more embodiments, BET BDII selective protein inhibitors exhibit an IL-22 IC50 of <about 250 nM, <about 50 nM, or <about 10 nM and/or an IL-17A IC50 of <about 250 nM, <about 50 nM, or <about 10 nM. In some embodiments, BET BDII selective protein inhibitors exhibit an IL-22 IC50 of <about 20 nM and or an IL-17A IC50 of <about 20 nM. In some embodiments, BET BDII selective protein inhibitors exhibit an IL-22 IC50 of <about 10 nM and or an IL-17A IC50 of <about 10 nM. In some embodiments, BET BDII selective protein inhibitors exhibit an IL-22 IC50 of <about 2 nM and or an IL-17A IC50 of <about 2 nM. In some embodiments, BET BDII selective protein inhibitors exhibit an IL-22 IC50 of <about 1 nM and or an IL-17A IC50 of <about 1 nM.

In some embodiments, BET BDII selective protein inhibitors with an IL-22 IC50 of <about 20 nM and or an IL-17A IC50 of <about 20 nM are promising drug candidates, but compounds having a lower activity may in some other embodiments be useful in particular contexts.

In one or more embodiments, BET BDII selective protein inhibitors exhibit a bioavailability of >about 12%, or >about 20%, or >about 25%, or >about 30%, or >about 40%, or >about 50%, or >about 60%, or >about 70%, or >about 80%, or >about 90%, or >about 95%. In some embodiments, BET BDII selective protein inhibitors exhibit a bioavailability of >about 20%, or >about 25%. In some embodiments, BET BDII selective protein inhibitors with a bioavailability of >about 55%.

In one or more embodiments, BET BDII selective protein inhibitors with a bioavailability of >about 20% are promising and >about 25% are advantageous drug candidates for oral administration, but compounds having a bioavailability of about 20% or less may in some embodiments be useful in particular contexts. In an embodiment, a compound having a bioavailability of >about 12% may be useful when administered orally.

In one or more embodiments, some compounds have two or more or all of the following characteristics an IL-22 IC50 of <about 20 nM, an IL-17A IC50 of <about 20 nM, a bioavailability of >about 12%, a rat microsomal stability of <about 4 ml/min/g liver, a rat microsomal stability of >about 20 minutes half-life in addition to having good activity and a selectivity of greater than about 1000-fold.

In one or more embodiments, some compounds have two or more or all of the following characteristics an IL-22 IC50 of <about 10 nM, an IL-17A IC50 of <about 10 nM, a bioavailability of >about 12%, or >about 25%, a rat microsomal stability of <about 4 ml/min/g liver, a rat microsomal stability of >about 20 minutes half-life in addition to having good activity and a selectivity of greater than about 2000-fold.

In one or more embodiments, some compounds have two or more or all of the following characteristics an IL-22 IC50 of <about 10 nM, an IL-17A IC50 of <about 10 nM, a bioavailability of >about 12%, or >about 25%, a rat microsomal stability of <about 4 ml/min/g liver, a rat microsomal stability of >about 20 minutes half-life in addition to having good activity of less than about 3 nM IC50 BD2 and a selectivity of greater than about 4000-fold.

In one or more embodiments, some compounds have two or more or all of the following characteristics an IL-22 IC50 of <about 10 nM, an IL-17A IC50 of <about 10 nM, a bioavailability of >about 12% or >about 25%, a rat microsomal stability of <about 4 ml/min/g liver, a rat microsomal stability of >about 20 minutes half-life in addition to having good activity of less than about 2 nM IC50 BD2 and a selectivity of greater than about 5000-fold.

In addition to the compounds showing activity and selectivity, other factors in selecting promising drug candidates can include for example, plasma stability, clearance, pK, and bioavailability. For drug candidates for oral delivery, a higher bioavailability can translate into a lower dosage and potentially fewer side effects, e.g., in the alimentary canal.

Compositions comprising a compound disclosed herein, or salt thereof (or combinations thereof) may, in one or more embodiments, be administered buccally, by inhalation (e.g., spray, nebulizer, or powder puff), epidural, by injection (including intraarticular, intravenous, intracoronary, subcutaneous, intramyocardial, intraperitoneal, intramuscular, intravascular or infusion), intradermal, intraperitoneal, intrapulmonary, intraarticular (e.g., injection), nasally, orally, parenterally, rectally, sublingually, topically, transdermally, vaginally, or via an implanted reservoir.

In some embodiments, the compounds disclosed herein, or salts thereof (or combinations thereof) are applied orally, for example as a solid dose form e.g., as a tablet, or a capsule, or as a semisolid or fluid dose form e.g., as a gel, or as liquid. In a fluid or semisolid dosage form the compound may in one or more embodiments be delivered as a suspension or as a solution.

In some embodiments, the compounds disclosed herein, or salts thereof (or combinations thereof) are applied by injection, e.g., as a solution or as a suspension. The solution or suspension may be in one or more embodiments, e.g., aqueous based, oil based, waterless, hydrophilic, hydrophobic, amphiphilic and/or an emulsion.

In some embodiments, the compounds disclosed herein, or salts thereof (or combinations thereof) are applied by inhalation, e.g., as a powder, spray or mist. In a fluid or liquid form, which can be used to form a mist (e.g., with a nebulizer) or spray (e.g., with an aerosol) the compound may in one or more embodiments be delivered as a suspension or as a solution.

In some embodiments, t h e compounds disclosed herein, or salts thereof (or combinations thereof) are applied topically, e.g., as a cream, emulsion, lotion, gel, ointment, mousse, foam, spray, or other topical dosage formats known in the art. In some embodiments when applied topically, the compounds disclosed herein may be effective where the compound is delivered primarily or substantially into the skin with low levels of transdermal penetration. In some embodiments, when applied topically the compounds disclosed herein may be effective where the compound is delivered primarily or substantially transdermally. In some embodiments, when applied topically the compounds disclosed herein may be effective where the compound is delivered intradermally and transdermally. In some embodiments, the penetration of the compound in the epidermis can be higher than that in the dermis. In some embodiments the penetration of the compound in the dermis can be higher than in the epidermis. In some embodiments the penetration of the compound in the dermis is similar to that in the epidermis. In some embodiments, the concentration of the compound per unit volume in the epidermis can be higher than that in the dermis. In some embodiments, the concentration of the compound per unit volume in the dermis can be higher than in the epidermis. In some embodiments, the concentration of the compound per unit volume in the dermis is similar to that in the epidermis.

Pharmaceutical compositions of the disclosure may be suitable for oral administration. Oral administration may be e.g., by solid e.g., tablet, caplet, capsule, by semi solid e.g., gel, or by liquid delivery forms e.g., syrup.

Tablets and similar formats contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, cornstarch, or alginic acid, binding agents, for example starch, gelatin, or acacia, and lubricating agents, for example magnesium stearate, stearic acid, or talc. The tablets may be uncoated, or they may be coated by known techniques to delay disintegration and absorption in the gastrointestinal tract and thereby provide a sustained action over a longer period. A tablet may be made by compressing or molding the active ingredient optionally with one or more pharmaceutically acceptable ingredients. Compressed tablets may be prepared by compressing, in a suitable machine, the active ingredient in a free-flowing form such as a powder or granules, optionally mixed with a binder, lubricant, inert diluent, surface active, or dispensing agent. Molded tablets may be made by molding, in a suitable machine, a mixture of the powdered active ingredient and a suitable carrier moistened with an inert liquid diluent.

Compositions for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with an appropriate hydrophilic medium or hydrophobic medium e.g., an oil medium, for example peanut oil, liquid paraffin, or olive oil. In particular, a pharmaceutical composition of the present invention may comprise a liquid-filled capsule dosage form in which the active ingredient is in suspension, part suspension or a solution in certain combinations of liquid and semi-solid excipients.

Compositions for oral administration may also be formulated as an aqueous or as a non-aqueous suspension of the active ingredient or formulated as an emulsion in which the active ingredient is suspended. The compositions contain the active ingredient in admixture with excipients suitable for the manufacture of such suspensions. Oily suspensions may be formulated by suspending the active ingredient in a suitable oil. Oil-in-water emulsions may also be employed. Dispersible powders and granules suitable for preparation of an aqueous/hydrophilic suspension by the addition of water/hydrophilic excipient provide the active ingredient in admixture with a dispersing or wetting agent, suspending agent and one or more preservatives.

The active ingredient of the present invention may be administered in an oral sustained release formulation. “Sustained release” refers to release of an active agent from a dosage form at a rate effective to achieve a therapeutic amount of the agent, or active metabolite thereof, in the systemic blood circulation over a prolonged period of time relative to that achieved by oral administration of a conventional formulation of the agent. Release of the agent occurs over an extended period of hours, for example, over a period of at least 6 hours, over a period of at least 8 hours, over a period of at least 12 hours, or over a period of at least 24 hours.

Pharmaceutical compositions of the disclosure may be suitable for topical or transdermal administration.

Examples of dosage forms for topical or transdermal administration of a compound disclosed herein or salt thereof include creams, drops, lotions, emulsions, foams, gels, inhalants, mousses, ointments, pastes, patches, powders, solutions, or sprays and the like. Suitable such topical formulations and dosage forms are described in Remington: The Science and Practice of Pharmacy (21st Edition, University of the Sciences in Philadelphia.

In some embodiments the compound is micronized when provided as a powder or as a suspension. In some embodiments the compound comprises nanoparticles.

In some embodiments, compositions comprising a novel compound disclosed herein or salt thereof (or combinations thereof) may be administered to young children. In some embodiments, compositions comprising a compound of the disclosure or salt thereof (or combinations thereof) may be administered to adolescents or teenagers. In some embodiments, compositions comprising a compound of the disclosure or salt thereof (or combinations thereof) may be administered to adults.

For drug candidates for oral delivery, a higher bioavailability can translate into a lower dosage and potentially fewer side effects, e.g., in the alimentary canal. For drug candidates for oral delivery, in some embodiments, a plasma concentration higher than the free EC50 for BD 2 for a sufficient period to have a therapeutic effect, e.g., in some embodiments a period of several hours, can translate into an effective drug. In some embodiments, oral delivery provides a plasma concentration over the free EC50 for BD 2 for a period of about 4 or more hours. In some embodiments, it is for a period of about 6 or more hours, or for a period of about 8 or more hours, or for a period of about 12 or more hours, or for a period of about 15 or more hours. In some embodiments, a therapeutically effective amount of drug is applied once a day. In some embodiments, a therapeutically effective amount of drug is applied two times a day, e.g., where the period in which the plasma concentration is higher than the free EC50 is less than 12 hours or less than 9 hours or less than 6 hours or between about 6 to about 12 hours or between about 9 to about 12 hours. In some embodiments, a therapeutically effective amount of drug is applied 3 times a day.

In some embodiments, compounds of the disclosure exhibit a microsomal half-life of >about 20 minutes, >about 30 minutes, >about 40 minutes, >about 50 minutes, >about 60 minutes, >about 80 minutes, >about 100 minutes, or >about 120 minutes or between about 20 to 180 minutes, or between about 60 to 180 minutes, or between about 120 to 180 minutes, or between about 100 to 150 minutes.

In some embodiments, compounds of the disclosure exhibit a thermodynamic solubility in Fasted State Simulated Intestinal Fluid (FaSSIF) pH 6.5 buffer of about 0.1 μM, >about 0.5 μM, >about 1 μM, >about 10 μM, >about 50 μM, >about 100 μM, >about 150 μM, or >about 200 μM, or between about 0.1 μM and about 200 μM, or between about 0.5 μM and about 100 μM or between about 1 μM and about 50 μM, or between about 0.1 μM and about 50 μM, or between about 0.5 μM and about 10 μM or between about 1 μM and about 10 μM.

In one or more embodiments, compounds of the disclosure exhibit a bioavailability of >about 10%, >about 12%, >about 20%, >about 25%, >about 30%, >about 40%, >about 50%, >about 60%>about 70%, >about 80%, >about 90%, or >about 95% or between about 20% to about 95%, or between about 20% to about 95% or between about 20% to about 80%. As used herein, bioavailability is the fraction of administered drug that reaches the systemic circulation (blood).

In one or more embodiments, BET BDII selective protein inhibitors exhibit an IL-22 IC50 of <about 250 nM, <about 50 nM, or <about 10 nM and/or an IL-17A IC50 of <about 250 nM, <about 50 nM, or <about 10 nM. In some embodiments, BET BDII selective protein inhibitors exhibit an IL-22 IC50 of <about 20 nM and/or an IL-17A IC50 of <about 20 nM. In some embodiments, BET BDII selective protein inhibitors exhibit an IL-22 IC50 of <about 10 nM and/or an IL-17A IC50 of <about 10 nM. In some embodiments, BET BDII selective protein inhibitors exhibit an IL-22 IC50 of <about 2 nM and/or an IL-17A IC50 of <about 2 nM. In some embodiments, BET BDII selective protein inhibitors exhibit an IL-22 IC50 of <about 1 nM and/or an IL-17A IC50 of <about 1 nM. In some embodiments, BET BDII selective protein inhibitors exhibit an IL-22 IC50 of between about 10 nM and about 0.1 nM, and/or an IL-17A IC50 of between about 10 nM and about 0.1 nM. In some embodiments, BET BDII selective protein inhibitors exhibit an IL-22 IC50 of between about 5 nM and about 0.1 nM, and/or an IL-17A IC50 of between about 5 nM and about 0.1 nM. In some embodiments, the IC50 is a mean of two or more measurements.

This disclosure includes the following numbered embodiments:
1. A compound of formula (1), a tautomer, a stereoisomer or a mixture of stereoisomers, a pharmaceutically acceptable salt, a hydrate, a deuterated derivative, or an N-oxide thereof:

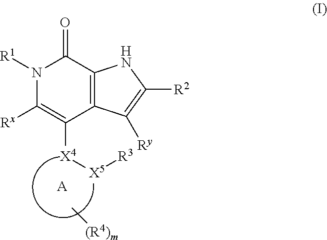

wherein:
Ring A is independently selected from phenyl, 5-membered heterocyclyl, and 6-membered heterocyclyl, wherein X⁴ and X⁵ are independently selected from carbon and nitrogen;
R¹ is independently selected from C₁-C₆-alkyl, C₁-C₅-haloalkyl, C₂-C₆-alkynyl, COR⁶, CO₂R⁶, C₁-C₄-alkylene-NR⁵R⁶, C₁-C₄-alkylene-OR⁷, C₁-C₄-alkyl-S(O)₂R⁶, C₃-C₆-cycloalkyl, aryl, heteroaryl, and 3- to 6-membered heterocycloalkyl;
R₂ is absent or independently selected from H, halo, cyano, nitro, SF₄, SF₅, ═O, S(O)₂R⁶, alkoxy, C₁-C₆-haloalkyl, C₁-C₈-alkyl, C₃-C₆-cycloalkyl, 4- to 8-membered heterocycloalkenyl, 3- to 8-membered heterocycloalkyl, aryl, and 5- or 6-membered heteroaryl,
   wherein the C₁-C₆-alkyl, alkoxy, C₃-C₆-cycloalkyl, 4- to 8-membered heterocycloalkenyl, 3- to 8-membered heterocycloalkyl, aryl, and 5- or 6-membered heteroaryl are independently optionally substituted with one or more groups selected from halogen, cyano, nitro, hydroxy, SF₅, amide, ester, alkoxy, and C₁-C₄-alkyl;
R³ is independently selected from R^{3a} and OR^{3b};
R^{3a} is independently selected from H, CN, C₁-C₄-alkyl, C₂-C₄-alkenyl, C₂-C₄-alkynyl, C₁-C₄-haloalkyl, C₂-C₄-haloalkenyl, C₃-C₈-cycloalkyl, C₅—Cs-cycloalkenyl, 5- to 9-membered heterocycloalkenyl, 3- to 9-membered heterocycloalkyl, phenyl, and 5- to 9-membered heteroaryl;
   wherein the cycloalkyl, heterocycloalkyl, cycloalkenyl, and heterocycloalkenyl are independently optionally substituted with from 1 to 4 R⁸ groups; and
   wherein the phenyl and the 5- to 9-membered heteroaryl are independently optionally substituted with from 1 to 5 R⁹ groups;
R^{3b} is independently selected from C₁-C₄-alkyl, C₂-C₄-alkylene-O—C₁-C₄-alkyl, C₁-C₄-haloalkyl, C₃-C₈-cycloalkyl, 3- to 8-membered heterocycloalkyl, phenyl, and 5- or 6-membered heteroaryl;
   wherein the cycloalkyl and 3- to 8-membered heterocycloalkyl are independently optionally substituted with from 1 to 4 R⁸ groups; and
   wherein the phenyl and the 5- or 6-membered heteroaryl are independently optionally substituted with from 1 to 5 R⁹ groups;
R⁴ is independently at each occurrence selected from H, ═O, ═S, halo, nitro, cyano, C₀-C₄-alkylene-NR⁵R⁶, —NR⁵R⁶, C₀-C₄-alkylene-OR⁷, —OR⁷, SR⁶, SOR⁶, C₀-C₄-alkylene-S(O)₂R⁶, —S(O)₂R⁶, SO₂NR⁶R⁶, C₀-C₄-alkylene-CO₂R⁶, —CO₂R⁶, C₀-C₄-alkylene-C(O)R⁶, —C(O)R⁶, C₀-C₄-alkylene-CONR⁶R⁶, —CONR⁶R⁶, C₁-C₆-alkyl, C₁-C₄-alkyl-S(O)₂R⁶, C₂-C₄-alkenyl, C₂-C₄-alkynyl, C₁-C₆-haloalkyl, C₃-C₆-cycloalkyl, aryl, 4- to 6-membered heterocycloalkyl, and 5- or 6-membered heteroaryl;
   wherein the aryl, 4- to 6-membered heterocycloalkyl, and 5- or 6-membered heteroaryl are independently optionally substituted with from 1 to 5 R⁹ groups;
R⁵ is independently at each occurrence selected from H, C₁-C₄-alkyl, C(O)—C₁-C₄-alkyl, and S(O)₂—C₁-C₄-alkyl; or R⁵ and R⁶, together with the nitrogen atom to which they are attached, form a C₅-C₈-heterocycloalkyl group optionally substituted with from 1 to 4 R⁸ groups;
R⁶ is independently at each occurrence selected from H and C₁-C₆-alkyl; or where two R⁶ groups are attached to the same nitrogen, those two R⁶ groups, together with the nitrogen atom to which they are attached, optionally form a C₅-C₈-heterocycloalkyl group optionally substituted with from 1 to 4 R⁸ groups;
R⁷ is independently at each occurrence selected from H, C₁-C₄-alkyl, C(O)—C₁-C₄-alkyl, and C₁-C₄-haloalkyl;
R⁸ is independently at each occurrence selected from ═O, ═S, fluoro, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl, 4- to 6-membered heterocycloalkyl, and C₃-C₆-cycloalkyl;
R⁹ is independently at each occurrence selected from halo, nitro, cyano, NR⁵R⁶, C₁-C₄-alkyl-OR⁷, OR⁷, SR⁶, SOR⁶, C₁-C₄-alkyl-S(O)₂R⁶, S(O)₂R⁶, SO₂NR⁶R⁶, CO₂R⁶, C(O)R⁶, CONR⁶R⁶, C₀-C₄-alkylene-R^{9a}, C₁-C₄-alkyl, C₂-C₄-alkenyl, C₂-C₄-alkynyl, C₁-C₄-haloalkyl, 4- to 6-membered heterocycloalkyl, and C₃-C₆-cycloalkyl; or where two R⁹ groups are attached to adjacent atoms, those two R⁹ groups, together with the atoms to which they are attached, optionally form a 5- or 6-membered heterocycloalkyl group optionally substituted with from 1 to 4 R⁸ groups;
R^{9a} is independently at each occurrence selected from 4- to 6-membered heterocycloalkyl;
R¹⁰ is absent or is independently at each occurrence selected from H, halo, C₁-C₆-alkyl, C₁-C₆-haloalkyl, nitro, cyano, NR⁶R⁶, OR⁷, SR⁶, SOR⁶, S(O)₂R⁶, SO₂NR⁶R⁶, CO₂R⁶, C(O)R⁶, CONR⁶R⁶, C₁-C₆-alkyl, C₂-C₆-alkenyl, C₂-C₆-alkynyl and 3- to 8-membered heterocycloalkyl;
R¹¹ is independently selected from phenyl, heteroaryl, and heterocyclyl, each optionally substituted with 1 to 4 R² groups and 1 to 3 R¹⁰ groups;
R^{x} and R^{y} are each independently selected from H, halo, nitro, cyano, C₁-C₆-alkylene-NR⁵R⁶, NR⁵R⁶, C₁-C₆-alkylene-OR⁷, C₁-C₆-alkyl-OR⁷, OR⁷, C₁-C₆-alkyl-SR⁶, SR⁶, C₁-C₆-alkyl-SOR⁶, SOR⁶, C₁-C₆-alkyl-S(O)₂R⁶, S(O)₂R⁶, C₁-C₆-alkyl-SO₂NR⁶R⁶, SO₂NR⁶R⁶, C₁-C₆-alkyl-CO₂R⁶, CO₂R⁶, C₁-C₆-alkyl-C(O)R⁶, C(O)R⁶, C₁-C₆-alkyl-CONR⁶R⁶, CONR⁶R⁶, C₁-C₆-alkyl, C₂-C₆-alkenyl, C₂-C₆-alkynyl, C₁-C₆-haloalkyl, C₃-C₈-cycloalkyl, 3- to 8-membered heterocycloalkyl, aryl, aryloxy, and 5- to 8-membered heteroaryl;
m is an integer selected from 0, 1, 2, 3 and 4;
wherein any of the aforementioned alkyl, alkylene, alkenyl, or C₃-C₆-cycloalkyl groups is optionally substituted, where chemically possible, by 1 to 5 substituents which are each independently at each occurrence selected from the group consisting of: C₁-C₄-alkyl, oxo, fluoro, nitro, cyano, NR^{a}R^{b}, OR^{a}, SR^{a}, CO₂R^{a}, C(O)R^{a}, CONR^{a}R^{a}, S(O)R^{a} and S(O)₂R^{a};
   wherein R^{a} is independently at each occurrence selected from H, C₁-C₄-alkyl and C₁-C₄-haloalkyl; and
   wherein R^{b} is independently at each occurrence selected from H, C₁-C₄-alkyl, C(O)—C₁-C₄-alkyl and S(O)₂—C₁-C₄-alkyl.
2. The compound of formula (1), a tautomer, a stereoisomer or a mixture of stereoisomers, a pharmaceutically acceptable salt, a hydrate, a deuterated derivative, or an N-oxide thereof,
wherein:
Ring A is a 6-membered heterocyclyl, wherein X⁴ and X⁵ are independently selected from carbon and nitrogen;
R¹ is independently selected from C₁-C₃-alkyl, C₁-C₃-haloalkyl, C₂-C₄-alkynyl, C₃-C₅-cycloalkyl, and 3- to 5-membered heterocycloalkyl;
R₂ is independently selected from H, halo, cyano, nitro, SF₄, SF₅, ═O, C₁-C₃-haloalkyl, C₁-C₃-alkyl, and C₃-C₅-cycloalkyl; S(O)₂R⁶, alkoxy, 4- to 5-membered heterocycloalkenyl, and 3- to 5-membered heterocycloalkyl,
   wherein the C₁-C₃-alkyl, alkoxy, C₃-C₅-cycloalkyl, 4- to 6-membered heterocycloalkenyl, and 3- to 5-membered heterocycloalkyl are independently optionally substituted with one or more groups selected from halogen, cyano, nitro, hydroxy, SF₅, amide, ester, alkoxy, and C₁-C₄-alkyl;
R³ is independently selected from R^{3a} and OR^{3b};
R^{3a} is independently selected from H, CN, C₁-C₄-alkyl, C₂-C₄-alkenyl, C₂-C₄-alkynyl, C₁-C₄-haloalkyl, C₂-C₄-haloalkenyl, C₃-C₈-cycloalkyl, C₅-C₈-cycloalkenyl, 5- to 9-membered heterocycloalkenyl, 3- to 9-membered heterocycloalkyl, phenyl, and 5- to 9-membered heteroaryl;
   wherein the cycloalkyl, heterocycloalkyl, cycloalkenyl, and heterocycloalkenyl are independently optionally substituted with from 1 to 4 R⁵ groups; and
   wherein the phenyl and the 5- to 9-membered heteroaryl are independently optionally substituted with from 1 to 5 R⁹ groups;
R^{3b} is independently selected from C₁-C₄-alkyl, C₂-C₄-alkylene-O—C₁-C₄-alkyl, C₁-C₄-haloalkyl, C₃-C₈-cycloalkyl, 3- to 8-membered heterocycloalkyl, phenyl, and 5- or 6-membered heteroaryl;
   wherein the cycloalkyl and 3- to 8-membered heterocycloalkyl are independently optionally substituted with from 1 to 4 R⁸ groups; and
   wherein the phenyl and the 5- or 6-membered heteroaryl are independently optionally substituted with from 1 to 5 R⁹ groups;
R⁴ is independently at each occurrence selected from H, ═O, ═S, halo, nitro, cyano, C₀-C₄-alkylene-NR⁵R⁶, —NR⁵R⁶, C₀-C₄-alkylene-OR⁷, —OR⁷, SR⁶, SOR⁶, C₀-C₄-alkylene-S(O)₂R⁶, —S(O)₂R⁶, SO₂NR⁶R⁶, C₀-C₄-alkylene-CO₂R⁶, —CO₂R⁶, C₀-C₄-alkylene-C(O)R⁶, —C(O)R⁶, C₀-C₄-alkylene-CONR⁶R⁶, —CONR⁶R⁶, C₁-C₆-alkyl, C₀-C₄-alkyl-S(O)₂R⁶, C₂-C₄-alkenyl, C₂-C₄-alkynyl, C₁-C₆-haloalkyl, C₃-C₆-cycloalkyl, aryl, 4- to 6-membered heterocycloalkyl, and 5- or 6-membered heteroaryl;
   wherein the aryl, 4- to 6-membered heterocycloalkyl, and 5- or 6-membered heteroaryl are independently optionally substituted with from 1 to 5 R⁹ groups;
R⁵ is independently at each occurrence selected from H, C₁-C₄-alkyl, C(O)—C₁-C₄-alkyl, and S(O)₂—C₁-C₄-alkyl; or R⁵ and R⁶, together with the nitrogen atom to which they are attached, form a C₅—Ca-heterocycloalkyl group optionally substituted with from 1 to 4 R⁸ groups;
R⁶ is independently at each occurrence selected from H and C₁-C₆-alkyl; or where two R⁶ groups are attached to the same nitrogen, those two R⁶ groups, together with the nitrogen atom to which they are attached, optionally form a C₅-C₈-heterocycloalkyl group optionally substituted with from 1 to 4 R⁸ groups;
R⁷ is independently at each occurrence selected from H, C₁-C₄-alkyl, C(O)—C₁-C₄-alkyl, and C₁-C₄-haloalkyl;
R⁸ is independently at each occurrence selected from ═O, ═S, fluoro, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl, 4- to 6-membered heterocycloalkyl, and C₃-C₆-cycloalkyl;
R⁹ is independently at each occurrence selected from halo, nitro, cyano, NR⁵R⁶, C₁-C₄-alkyl-OR⁷, OR⁷, SR⁶, SOR⁶, C₁-C₄-alkyl-S(O)₂R⁶, S(O)₂R⁶, SO₂NR⁶R⁶, CO₂R⁶, C(O)R⁶, CONR⁶R⁶, C₀-C₄-alkylene-R^{9a}, C₁-C₄-alkyl, C₂-C₄-alkenyl, C₂-C₄-alkynyl, C₁-C₄-haloalkyl, 4- to 6-membered heterocycloalkyl, and C₃-C₆-cycloalkyl; or where two R⁹ groups are attached to adjacent atoms, those two R⁹ groups, together with the atoms to which they are attached, optionally form a 5- or 6-membered heterocycloalkyl group optionally substituted with from 1 to 4 R⁸ groups;
R^{9a} is independently at each occurrence selected from 4- to 6-membered heterocycloalkyl;
R^{1O} is absent or is independently at each occurrence selected from H, halo, C₁-C₆-alkyl, C₁-C₆-haloalkyl, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₆-alkyl, C₂-C₆-alkenyl, C₂-C₆-alkynyl and 3- to 8-membered heterocycloalkyl;
R¹¹ is independently selected from phenyl, heteroaryl, and heterocyclyl, each optionally substituted with 1 to 2 R² groups and 1 to 2 R¹⁰ groups;
R^{x} and R^{y} are each independently selected from H, halo, nitro, cyano, C₁-C₄-alkylene-NR⁵R⁶, NR⁵R⁶, C₁-C₄-alkylene-OR⁷, C₁-C₄-alkyl-OR⁷, OR⁷, C₁-C₄-alkyl-SR⁶, SR⁶, C₁-C₄-alkyl-SOR⁶, SOR⁶, C₁-C₄-alkyl-S(O)₂R⁶, S(O)₂R⁶, C₁-C₄-alkyl-SO₂NR⁶R⁶, SO₂NR⁶R⁶, C₁-C₄-alkyl-CO₂R⁶, CO₂R⁶, C₁-C₄-alkyl-C(O)R⁶, C(O)R⁶, C₁-C₄-alkyl-CONR⁶R⁶, CONR⁶R⁶, C₁-C₄-alkyl, C₂-C₄-alkenyl, C₂-C₄-alkynyl, C₁-C₄-haloalkyl, C₃-C₆-cycloalkyl, 3- to 6-membered heterocycloalkyl, aryl, aryloxy, and 5- to 6-membered heteroaryl;
m is an integer selected from 0, 1, and 2;
wherein any of the aforementioned alkyl, alkylene, alkenyl, or C₃-C₆-cycloalkyl groups is optionally substituted, where chemically possible, by 1 to 3 substituents which are each independently at each occurrence selected from the group consisting of: C₁-C₄-alkyl, oxo, fluoro, nitro, cyano, NR^{a}R^{b}, OR^{a}, SR^{a}, CO₂R^{a}, C(O)R^{a}, CONR^{a}R^{a}, S(O)R^{a} and S(O)₂R^{a};
   wherein R^{a} is independently at each occurrence selected from H, C₁-C₃-alkyl and C₁-C₃-haloalkyl; and
   wherein R^{b} is independently at each occurrence selected from H, C₁-C₃-alkyl, C(O)—C₁-C₃-alkyl and S(O)₂—C₁-C₃-alkyl.
3. A compound of Formula (IA), or a tautomer, a stereoisomer or a mixture of stereoisomers, a pharmaceutically acceptable salt, a hydrate, a deuterated derivative, or an N-oxide thereof:

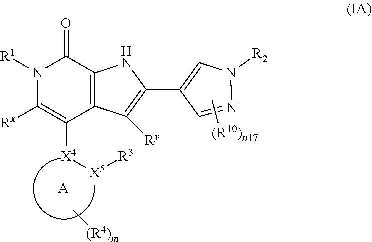

wherein X⁴, X⁵, R², R³, R⁴, and R¹⁰ are as defined in embodiment 1; and
wherein:
   R¹ is independently selected from C₁-C₄-alkyl;
   R^{x} and R^{y} are each independently selected from H, halo, nitro, cyano, C₁-C₆-alkyl, C₂-C₆-alkenyl, C₂-C₆-alkynyl, and C₁-C₆-haloalkyl;
   m is an integer selected from 0, 1, and 2; and
   n₁₇ is an integer selected from 0, 1 and 2.
4. A compound of any one of the preceding embodiments, a tautomer, a stereoisomer or a mixture of stereoisomers, a pharmaceutically acceptable salt, a hydrate, a deuterated derivative, or an N-oxide thereof, wherein Ring A is independently selected from 5- and 6-membered heteroaryls.
5. A compound of any one of the preceding embodiments, or a tautomer, a stereoisomer or a mixture of stereoisomers, a pharmaceutically acceptable salt, a hydrate, a deuterated derivative, or an N-oxide thereof,
wherein Ring A is

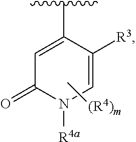

R^{4a} is selected from H, C₁-C₄-alkyl, C₃-C₃-cycloalkyl, and 4- to 6-membered heterocycloalkyl, wherein the C₁-C₄-alkyl, C₃-C₈-cycloalkyl, and 4- to 6-membered heterocycloalkyl are optionally independently substituted with C₁-C₃-alkyl, SR⁶, OR⁷, and —NR⁵R⁶; and
   R³, R⁴, and m are as defined in embodiment 1.
6. A compound of any one of the preceding embodiments, or a tautomer, a stereoisomer or a mixture of stereoisomers, a pharmaceutically acceptable salt, a hydrate, a deuterated derivative, or an N-oxide thereof, wherein R^{4a} is selected from methyl, cyclopropyl, oxetane, cyclobutanol, methylcyclobutane, ethyl morpholine, —CH₂—CH₂—OMe, —CH₂—CH₂—OH, —CH₂—CH₂—N(CH₃)₂, —CH₂—CH₂—S—CH₃, —CH₂—CH₂—O—CHF₂, and azetidine.
7. A compound of any one of the preceding embodiments, or a tautomer, a stereoisomer or a mixture of stereoisomers, a pharmaceutically acceptable salt, a hydrate, a deuterated derivative, or an N-oxide thereof, wherein R^{3a} and R^{3b} are phenyl independently optionally substituted with from 1 to 3 R⁹ groups.
8. A compound of any one of the preceding embodiments, or a tautomer, a stereoisomer or a mixture of stereoisomers, a pharmaceutically acceptable salt, a hydrate, a deuterated derivative, or an N-oxide thereof, wherein R^{x} is H, halogen, or C₁-C₄-alkyl.
9. A compound of any one of the preceding embodiments, or a tautomer, a stereoisomer or a mixture of stereoisomers, a pharmaceutically acceptable salt, a hydrate, a deuterated derivative, or an N-oxide thereof, wherein R^{y} is H, halogen, or C₁-C₃-alkyl.
10. A compound of Formula (IV), or a tautomer, a stereoisomer or a mixture of stereoisomers, a pharmaceutically acceptable salt, a hydrate, a deuterated derivative, or an N-oxide thereof:

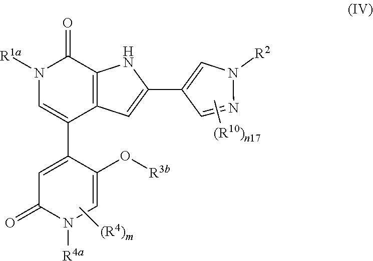

wherein:
   R^{1a} is selected from C₁-C₄-alkyl;
   m is an integer selected from 0, 1, and 2;
   n₁₇ is an integer selected from 0, 1 and 2.
   R^{4a} is selected from H, C₁-C₆-alkyl, C₁-C₆-haloalkyl, C₃-C₆-cycloalkyl, aryl, and 4- to 6-membered heterocycloalkyl, wherein the C₁-C₆-alkyl, C₁-C₆-haloalkyl, C₃-C₆-cycloalkyl, aryl, and 4- to 6-membered heterocycloalkyl are optionally independently substituted with C₁-C₃-alkyl, SR⁶, OR⁷, and —NR⁵R⁶; and
   R², R^{3b}, R⁴, R⁵, R⁶, R⁷, and R¹⁰ are as defined in embodiment 1.
11. A compound of Formula (V), or a tautomer, a stereoisomer or a mixture of stereoisomers, a pharmaceutically acceptable salt, a hydrate, a deuterated derivative, or an N-oxide thereof:

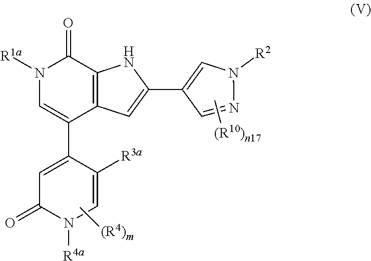

wherein:
   R₁ₐ is selected from C₁-C₄-alkyl;
   m is an integer selected from 0, 1, and 2;
   n₁₇ is an integer selected from 0, 1 and 2;
   R^{4a} is selected from H, C₁-C₆-alkyl, C₁-C₆-haloalkyl, C₃-C₆-cycloalkyl, aryl, and 4- to 6-membered heterocycloalkyl, wherein the C₁-C₆-alkyl, C₁-C₆-haloalkyl, C₃-C₆-cycloalkyl, aryl, and 4- to 6-membered heterocycloalkyl are optionally independently substituted with C₁-C₃-alkyl, SR⁶, OR⁷, and —NR⁵R⁶; and
   R², R^{3a}, R⁴, R⁵, R⁶, R⁷, and R¹⁰ are as defined in embodiment 1.
12. A compound of Formula (VA), or a tautomer, a stereoisomer or a mixture of stereoisomers, a pharmaceutically acceptable salt, a hydrate, a deuterated derivative, or an N-oxide thereof:

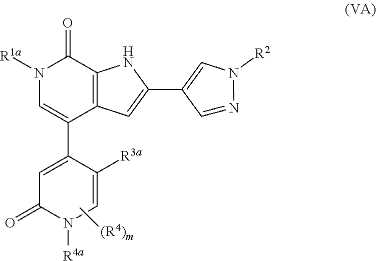

wherein:
R^{1a} is selected from C₁-C₃-alkyl;
m is an integer selected from 0 and 1;
R^{4a} is selected from H, C₁-C₆-alkyl, C₁-C₆-haloalkyl, C₃-C₆-cycloalkyl, aryl, and 4- to 6-membered heterocycloalkyl, wherein the C₁-C₆-alkyl, C₁-C₆-haloalkyl, C₃-C₆-cycloalkyl, aryl, and 4- to 6-membered heterocycloalkyl are optionally independently substituted with C₁-C₃-alkyl, SR⁶, OR⁷, and —NR⁵R⁶:
R² is C₁-C₄-haloalkyl; and
R^{3a}, R⁴, R⁵, R⁶, and Rare as defined in embodiment 1.
13. A compound of any one of the preceding embodiments, or a tautomer, a stereoisomer or a mixture of stereoisomers, a pharmaceutically acceptable salt, a hydrate, a deuterated derivative, or an N-oxide thereof, wherein Ring A is substituted on the nitrogen with 1 group selected from C₁-C₄-alkyl, cyclopropyl, cyclobutyl, methyl-cyclobutyl, and 4-membered heterocycloalkyl.
14. A compound of Formula (VI), or a tautomer, a stereoisomer or a mixture of stereoisomers, a pharmaceutically acceptable salt, a hydrate, a deuterated derivative, or an N-oxide thereof:

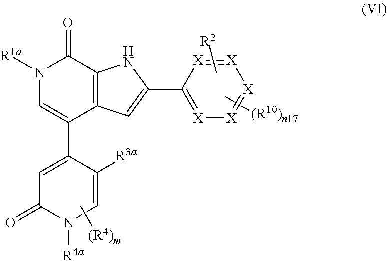

wherein:
   each X is independently selected from carbon and nitrogen;
   R^{1a} is selected from C₁-C₄-alkyl;
   m is an integer selected from 0, 1, and 2;
   n₁₇ is an integer selected from 0, 1 and 2;
   R^{4a} is selected from H, C₁-C₆-alkyl, C₁-C₆-haloalkyl, C₃-C₆-cycloalkyl, aryl, and 4- to 6-membered heterocycloalkyl, wherein the C₁-C₆-alkyl, C₁-C₆-haloalkyl, C₃-C₆-cycloalkyl, aryl, and 4- to 6-membered heterocycloalkyl are optionally independently substituted with C₁-C₃-alkyl, SR⁶, OR⁷, and —NR⁵R⁶; and
   R², R^{3a}, R⁴, R⁵, R⁶, R⁷, and R¹⁰ are as defined in embodiment 1.
15. A compound of Formula (VIA), or a tautomer, a stereoisomer or a mixture of stereoisomers, a pharmaceutically acceptable salt, a hydrate, a deuterated derivative, or an N-oxide thereof:

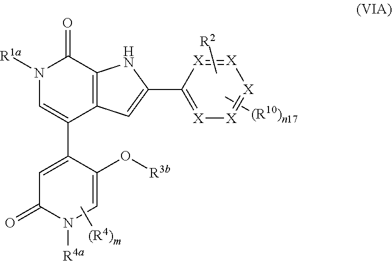

wherein:
   each X is independently selected from carbon and nitrogen;
   R₁ₐ is selected from C₁-C₄-alkyl;
   m is an integer selected from 0, 1, and 2;
   n₁₇ is an integer selected from 0, 1 and 2;
   R^{4a} is selected from H, C₁-C₆-alkyl, C₁-C₆-haloalkyl, C₃-C₆-cycloalkyl, aryl, and 4- to 6-membered heterocycloalkyl, wherein the C₁-C₆-alkyl, C₁-C₆-haloalkyl, C₃-C₆-cycloalkyl, aryl, and 4- to 6-membered heterocycloalkyl are optionally independently substituted with C₁-C₃-alkyl, SR⁶, OR⁷, and —NR⁶R⁶; and
   R², R^{3b}, R⁴, R⁵, R⁶, R⁷, and R¹⁰ are as defined in embodiment 1.
16. A compound of Formula (VIB), or a tautomer, a stereoisomer or a mixture of stereoisomers, a pharmaceutically acceptable salt, a hydrate, a deuterated derivative, or an N-oxide thereof:

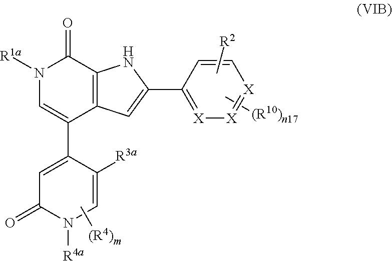

wherein:
   each X is independently selected from carbon and nitrogen;
   R^{1a} is selected from C₁-C₄-alkyl;
   m is an integer selected from 0, 1, and 2;
   n₁₇ is an integer selected from 0, 1 and 2;
   R^{4a} is selected from H, C₁-C₆-alkyl, C₁-C₆-haloalkyl, C₃-C₆-cycloalkyl, aryl, and 4- to 6-membered heterocycloalkyl, wherein the C₁-C₆-alkyl, C₁-C₃-haloalkyl, C₃-C₆-cycloalkyl, aryl, and 4- to 6-membered heterocycloalkyl are optionally independently substituted with C₁-C₃-alkyl, SR⁶, OR⁷, and —NR⁵R⁶; and
   R², R^{3a}, R⁴, R⁶, R⁶, R⁷, and R¹⁰ are as defined in embodiment 1.
17. A compound of Formula (VIC), or a tautomer, a stereoisomer or a mixture of stereoisomers, a pharmaceutically acceptable salt, a hydrate, a deuterated derivative, or an N-oxide thereof:

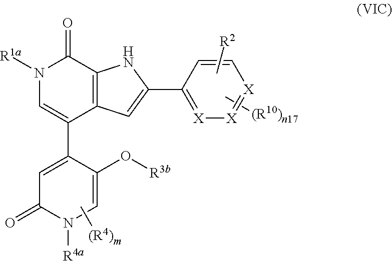

wherein:
   each X is independently selected from carbon and nitrogen;
   R^{1a} is selected from C₁-C₄-alkyl;
   m is an integer selected from 0, 1, and 2;
   n₁₇ is an integer selected from 0, 1 and 2;
   R^{4a} is selected from H, C₁-C₆-alkyl, C₁-C₆-haloalkyl, C₃-C₆-cycloalkyl, aryl, and 4- to 6-membered heterocycloalkyl, wherein the C₁-C₆-alkyl, C₁-C₆-haloalkyl, C₃-C₆-cycloalkyl, aryl, and 4- to 6-membered heterocycloalkyl are optionally independently substituted with C₁-C₃-alkyl, SR⁶, OR⁷, and —NR₅R⁶; and
   R², R^{3b}, R⁴, R⁵, R⁶, R⁷, and R¹⁰ are as defined in embodiment 1.
18. A compound of Formula (VID), or a tautomer, a stereoisomer or a mixture of stereoisomers, a pharmaceutically acceptable salt, a hydrate, a deuterated derivative, or an N-oxide thereof:

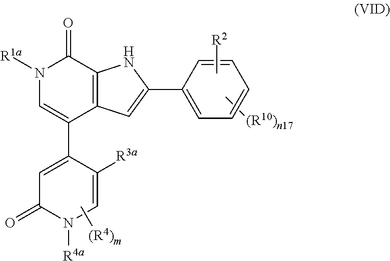

wherein:
   R^{1a} is selected from C₁-C₄-alkyl;
   m is an integer selected from 0, 1, and 2;
   n₁₇ is an integer selected from 0, 1 and 2;
   R^{4a} is selected from H, C₁-C₆-alkyl, C₁-C₆-haloalkyl, C₃-C₆-cycloalkyl, aryl, and 4- to 6-membered heterocycloalkyl, wherein the C₁-C₆-alkyl, C₁-C₆-haloalkyl, C₃-C₆-cycloalkyl, aryl, and 4- to 6-membered heterocycloalkyl are optionally independently substituted with C₁-C₃-alkyl, SR⁶, OR⁷, and —NR⁵R⁶; and
   R², R^{3a}, R⁴, R⁵, R⁶, R⁷, and R¹⁰ are as defined in embodiment 1.
19. A compound of Formula (VIE), or a tautomer, a stereoisomer or a mixture of stereoisomers, a pharmaceutically acceptable salt, a hydrate, a deuterated derivative, or an N-oxide thereof:

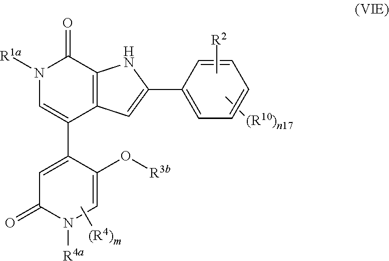

wherein:
   R^{1a} is selected from C₁-C₄-alkyl;
   m is an integer selected from 0, 1, and 2;
   n₁₇ is an integer selected from 0, 1 and 2;
   R^{4a} is selected from H, C₁-C₆-alkyl, C₁-C₆-haloalkyl, C₃-C₆-cycloalkyl, aryl, and 4- to 6-membered heterocycloalkyl, wherein the C₁-C₆-alkyl, C₁-C₆-haloalkyl, C₃-C₆-cycloalkyl, aryl, and 4- to 6-membered heterocycloalkyl are optionally independently substituted with C₁-C₃-alkyl, SR⁶, OR⁷, and —NR⁵R⁶; and
   R², R^{3b}, R⁴, R⁶, R⁶, R⁷, and R¹⁰ are as defined in embodiment 1.
20. A compound of Formula (VIF), or a tautomer, a stereoisomer or a mixture of stereoisomers, a pharmaceutically acceptable salt, a hydrate, a deuterated derivative, or an N-oxide thereof:

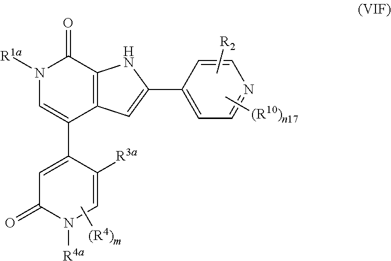

wherein:
   R^{1a} is selected from C₁-C₄-alkyl;
   m is an integer selected from 0, 1, and 2;
   n₁₇ is an integer selected from 0, 1 and 2;
   R^{4a} is selected from H, C₁-C₆-alkyl, C₁-C₆-haloalkyl, C₃-C₆-cycloalkyl, aryl, and 4- to 6-membered heterocycloalkyl, wherein the C₁-C₆-alkyl, C₁-C₆-haloalkyl, C₃-C₆-cycloalkyl, aryl, and 4- to 6-membered heterocycloalkyl are optionally independently substituted with C₁-C₃-alkyl, SR⁶, OR⁷, and —NR⁵R⁶; and
   R², R^{3a}, R⁴, R⁶, R⁶, R⁷, and R¹⁰ are as defined in embodiment 1.
21. A compound of Formula (VIG), or a tautomer, a stereoisomer or a mixture of stereoisomers, a pharmaceutically acceptable salt, a hydrate, a deuterated derivative, or an N-oxide thereof:

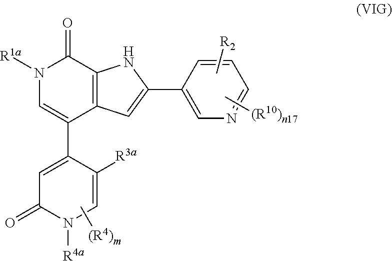

wherein:
R^{1a} is selected from C₁-C₄-alkyl;
m is an integer selected from 0, 1, and 2;
n₁₇ is an integer selected from 0, 1 and 2;
R^{4a} is selected from H, C₁-C₆-alkyl, C₁-C₆-haloalkyl, C₃-C₆-cycloalkyl, aryl, and 4- to 6-membered heterocycloalkyl, wherein the C₁-C₆-alkyl, C₁-C₆-haloalkyl, C₃-C₆-cycloalkyl, aryl, and 4- to 6-membered heterocycloalkyl are optionally independently substituted with C₁-C₃-alkyl, SR⁶, OR⁷, and —NR⁵R⁶ and R², R^{3a}, R⁴, R⁵, R⁶, R⁷, and R¹⁰ are as defined in embodiment 1.
22. A compound of Formula (VIH), or a tautomer, a stereoisomer or a mixture of stereoisomers, a pharmaceutically acceptable salt, a hydrate, a deuterated derivative, or an N-oxide thereof:

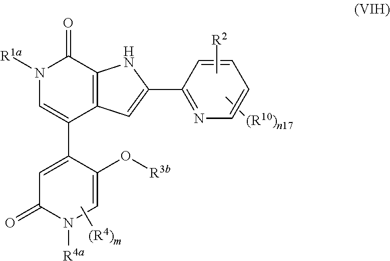

wherein:
R^{1a} is selected from C₁-C₄-alkyl;
m is an integer selected from 0, 1, and 2;
n₁₇ is an integer selected from 0, 1 and 2;
R^{4a} is selected from H, C₁-C₆-alkyl, C₁-C₆-haloalkyl, C₃-C₆-cycloalkyl, aryl, and 4- to 6-membered heterocycloalkyl, wherein the C₁-C₆-alkyl, C₁-C₆-haloalkyl, C₃-C₆-cycloalkyl, aryl, and 4- to 6-membered heterocycloalkyl are optionally independently substituted with C₁-C₃-alkyl, SR⁶, OR⁷, and —NR⁵R⁶; and
R², R^{3b}, R⁴, R⁵, R⁶, R⁷, and R¹⁰ are as defined in embodiment 1.
23. A compound of Formula (VIi), or a tautomer, a stereoisomer or a mixture of stereoisomers, a pharmaceutically acceptable salt, a hydrate, a deuterated derivative, or an N-oxide thereof:

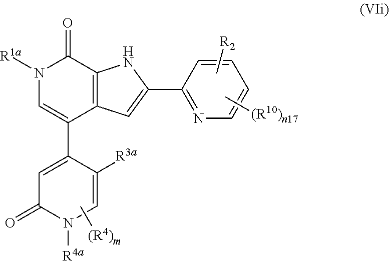

wherein:
R^{1a} is selected from C₁-C₄-alkyl;
m is an integer selected from 0, 1, and 2;
n₁₇ is an integer selected from 0, 1 and 2;
R^{4a} is selected from H, C₁-C₆-alkyl, C₁-C₆-haloalkyl, C₃-C₆-cycloalkyl, aryl, and 4- to 6-membered heterocycloalkyl, wherein the C₁-C₆-alkyl, C₁-C₆-haloalkyl, C₃-C₆-cycloalkyl, aryl, and 4- to 6-membered heterocycloalkyl are optionally independently substituted with C₁-C₃-alkyl, SR⁶, OR⁷, and —NR⁵R⁶; and
R², R^{3a}, R⁴, R⁶, R⁶, R⁷, and R^{1O} are as defined in embodiment 1.
24. A compound of Formula (VII), or a tautomer, a stereoisomer or a mixture of stereoisomers, a pharmaceutically acceptable salt, a hydrate, a deuterated derivative, or an N-oxide thereof:

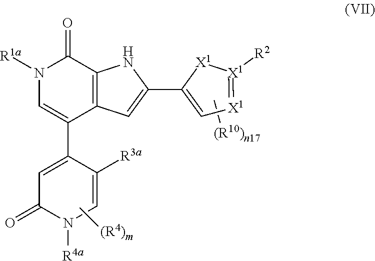

wherein:
   X¹ is independently selected from carbon, sulfur, and nitrogen;
   R^{1a} is selected from C₁-C₄-alkyl;
   m is an integer selected from 0, 1, and 2;
   n₁₇ is an integer selected from 0, 1 and 2;
   R^{4a} is selected from H, C₁-C₆-alkyl, C₁-C₆-haloalkyl, C₃-C₆-cycloalkyl, aryl, and 4- to 6-membered heterocycloalkyl, wherein the C₁-C₆-alkyl, C₁-C₆-haloalkyl, C₃-C₆-cycloalkyl, aryl, and 4- to 6-membered heterocycloalkyl are optionally independently substituted with C₁-C₃-alkyl, SR⁶, OR⁷, and —NR⁵R⁶; and
   R², R³, R⁴, R⁶, R⁶, R⁷, and R¹⁰ are as defined in embodiment 1.
25. A compound of Formula (Vill), or a tautomer, a stereoisomer or a mixture of stereoisomers, a pharmaceutically acceptable salt, a hydrate, a deuterated derivative, or an N-oxide thereof:

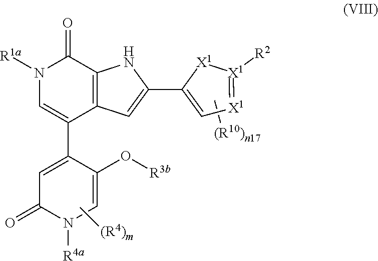

wherein:
   X¹ is independently selected from carbon, sulfur and nitrogen;
   R^{1a} is selected from C₁-C₄-alkyl;
   m is an integer selected from 0, 1, and 2;
   n₁₇ is an integer selected from 0, 1 and 2;
   R^{4a} is selected from H, C₁-C₆-alkyl, C₁-C₆-haloalkyl, C₃-C₆-cycloalkyl, aryl, and 4- to 6-membered heterocycloalkyl, wherein the C₁-C₆-alkyl, C₁-C₆-haloalkyl, C₃-C₆-cycloalkyl, aryl, and 4- to 6-membered heterocycloalkyl are optionally independently substituted with C₁-C₃-alkyl, SR⁶, OR⁷, and —NR⁵R⁶; and
   R², R^{3b}, R⁴, R⁶, R⁶, R⁷, and R¹⁰ are as defined in embodiment 1.
26. A compound of Formula (VIIIA), or a tautomer, a stereoisomer or a mixture of stereoisomers, a pharmaceutically acceptable salt, a hydrate, a deuterated derivative, or an N-oxide thereof:

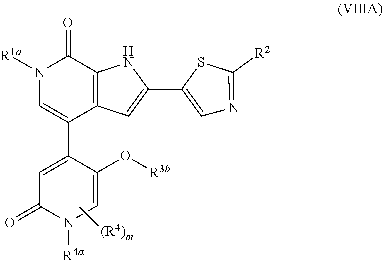

wherein:
R^{1a} is selected from C₁-C₄-alkyl;
m is an integer selected from 0, 1, and 2;
R^{4a} is selected from H, C₁-C₆-alkyl, C₁-C₆-haloalkyl, C₃-C₆-cycloalkyl, aryl, and 4- to 6-membered heterocycloalkyl, wherein the C₁-C₆-alkyl, C₁-C₆-haloalkyl, C₃-C₆-cycloalkyl, aryl, and 4- to 6-membered heterocycloalkyl are optionally independently substituted with C₁-C₃-alkyl, SR⁶, OR⁷, and —NR⁵R⁶ and
R², R^{3b}, R⁴, R⁵, R⁶, and R⁷ are as defined in embodiment 1.
27. A compound of any one of the preceding embodiments, or a tautomer, a stereoisomer or a mixture of stereoisomers, a pharmaceutically acceptable salt, a hydrate, a deuterated derivative, or an N-oxide thereof, wherein R² is independently selected from halogen, C₁-C₆-alkyl, alkoxy, C₁-C₆-haloalkyl, ethyl, cyano, nitro isopropyl, tert-butyl, cyclopropyl, SF₄ and SF₅.
28. A compound of any previous embodiment, or a tautomer, a stereoisomer or a mixture of stereoisomers, a pharmaceutically acceptable salt, a hydrate, a deuterated derivative, or an N-oxide thereof, wherein R² is independently selected from C₁-C₄-haloalkyl, ethyl, cyano, nitro, isopropyl, tert-butyl, cyclopropyl, SF₄, and SF₅.
29. A compound of any one of the preceding embodiments, or a tautomer, a stereoisomer or a mixture of stereoisomers, a pharmaceutically acceptable salt, a hydrate, a deuterated derivative, or an N-oxide thereof, wherein R² is independently selected from halogen, C₁-C₆-alkyl, alkoxy, and C₁-C₆-haloalkyl.
30. A compound of any one of the preceding embodiments, or a tautomer, a stereoisomer or a mixture of stereoisomers, a pharmaceutically acceptable salt, a hydrate, a deuterated derivative, or an N-oxide thereof, wherein R² is independently selected from CF₃, CHF₂, CH₂CF₃, and CH₂CH₂F.
31. A compound of any one of the preceding embodiments, or a tautomer, a stereoisomer or a mixture of stereoisomers, a pharmaceutically acceptable salt, a hydrate, a deuterated derivative, or an N-oxide thereof, wherein R² is independently selected from CBr₃, CHBr₂, CH₂CBr₃, and CH₂CH₂Br.
32. A compound of any one of the preceding embodiments, or a tautomer, a stereoisomer or a mixture of stereoisomers, a pharmaceutically acceptable salt, a hydrate, a deuterated derivative, or an N-oxide thereof, wherein R² is independently selected from CCl₃, CHCl₂, CH₂CCl₃, and CH₂CH₂Cl.
33. A compound of any one of the preceding embodiments, or a tautomer, a stereoisomer or a mixture of stereoisomers, a pharmaceutically acceptable salt, a hydrate, a deuterated derivative, or an N-oxide thereof, wherein R₂ is independently selected from —CH₂—CH₂—OCF₂, —CH₂—CH₂—OCF₃, SF₄, or SF₅.
34. A compound of any one of the preceding embodiments, or a tautomer, a stereoisomer or a mixture of stereoisomers, a pharmaceutically acceptable salt, a hydrate, a deuterated derivative, or an N-oxide thereof, wherein R² is not CH(CH₃)₂, CH₃, or oxetanyl.
35. A compound of any one of the preceding embodiments, or a tautomer, a stereoisomer or a mixture of stereoisomers, a pharmaceutically acceptable salt, a hydrate, a deuterated derivative, or an N-oxide thereof, wherein R¹ is selected from methyl, ethyl, and C₁-C₄-haloalkyl.
36. A compound of any one of the preceding embodiments, or a tautomer, a stereoisomer or a mixture of stereoisomers, a pharmaceutically acceptable salt, a hydrate, a deuterated derivative, or an N-oxide thereof, wherein R^{1a} is selected from methyl, ethyl, and C₁-C₄-haloalkyl.
37. A compound of any one of the preceding embodiments, or a tautomer, a stereoisomer or a mixture of stereoisomers, a pharmaceutically acceptable salt, a hydrate, a deuterated derivative, or an N-oxide thereof, wherein n₁₇ is 0 or 1.
38. A compound of any one of the preceding embodiments, or a tautomer, a stereoisomer or a mixture of stereoisomers, a pharmaceutically acceptable salt, a hydrate, a deuterated derivative, or an N-oxide thereof, wherein if n is 1, then R¹⁰ is not CH₃ or cyano.
39. A compound of any one of the preceding embodiments, a tautomer, a stereoisomer or a mixture of stereoisomers, a pharmaceutically acceptable salt, a hydrate, a deuterated derivative, or an N-oxide thereof, comprising at least one halogen, or at least two halogens, or at least three halogens.
40. A compound of any one of the preceding embodiments, wherein the compound is selected from:

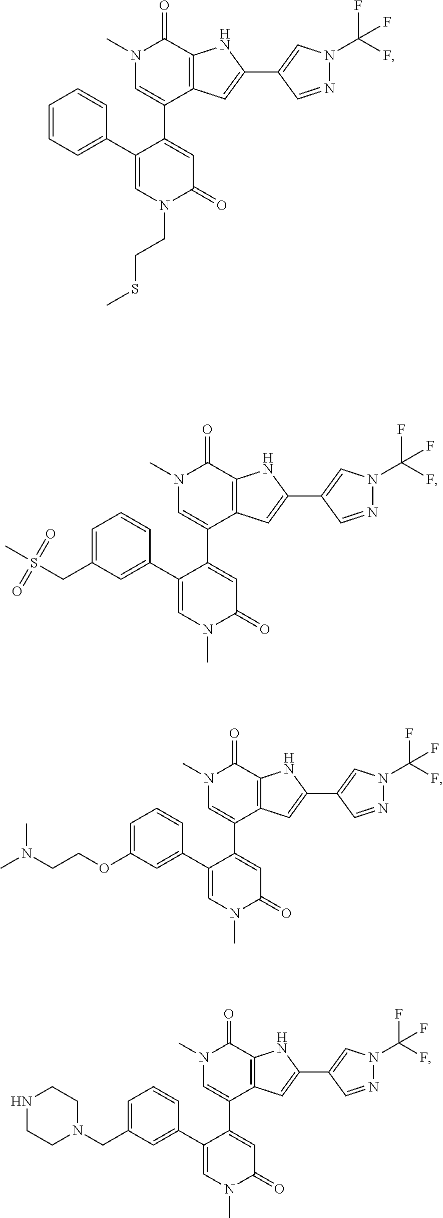 [SMILES: CSCCn1cc(-c2ccccc2)c(-c2cn(C)c(=O)c3[nH]c(-c4cnn(C(F)(F)F)c4)cc23)cc1=O ; Cn1cc(-c2cccc(CS(C)(=O)=O)c2)c(-c2cn(C)c(=O)c3[nH]c(-c4cnn(C(F)(F)F)c4)cc23)cc1=O ; CN(C)CCOc1cccc(-c2cn(C)c(=O)cc2-c2cn(C)c(=O)c3[nH]c(-c4cnn(C(F)(F)F)c4)cc23)c1 ; Cn1cc(-c2cccc(CN3CCNCC3)c2)c(-c2cn(C)c(=O)c3[nH]c(-c4cnn(C(F)(F)F)c4)cc23)cc1=O]

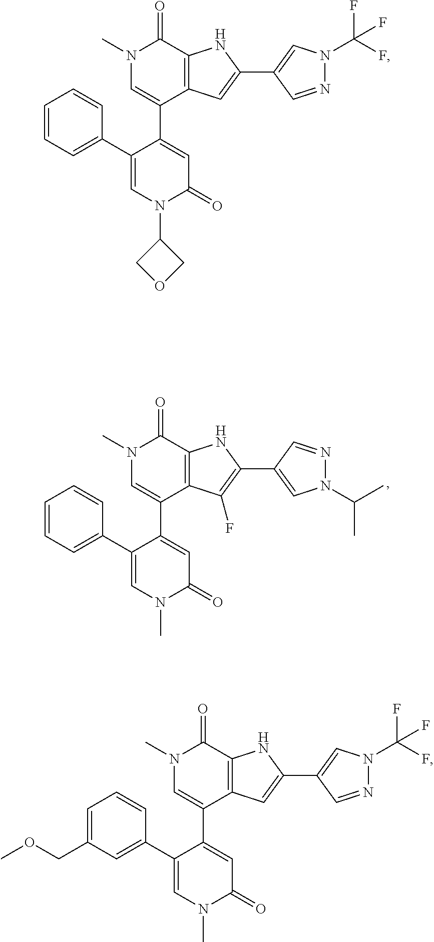

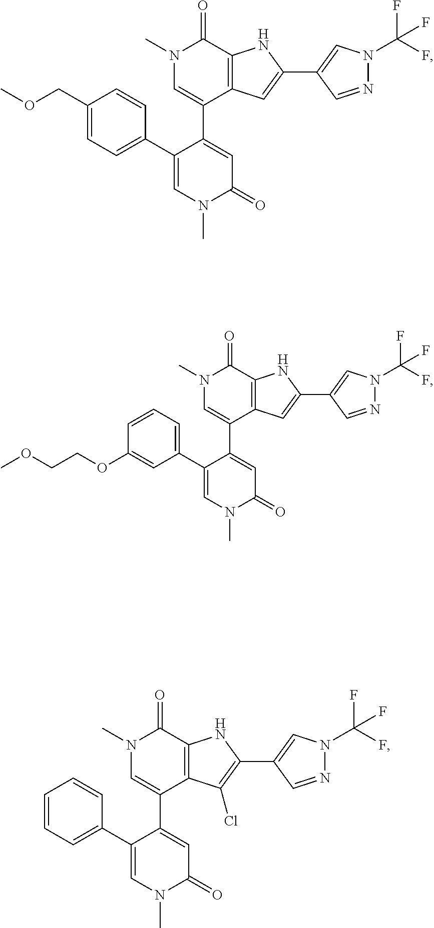 [SMILES: COCc1ccc(-c2cn(C)c(=O)cc2-c2cn(C)c(=O)c3[nH]c(-c4cnn(C(F)(F)F)c4)cc23)cc1 ; COCCOc1cccc(-c2cn(C)c(=O)cc2-c2cn(C)c(=O)c3[nH]c(-c4cnn(C(F)(F)F)c4)cc23)c1 ; Cn1cc(-c2ccccc2)c(-c2cn(C)c(=O)c3[nH]c(-c4cnn(C(F)(F)F)c4)c(Cl)c23)cc1=O]

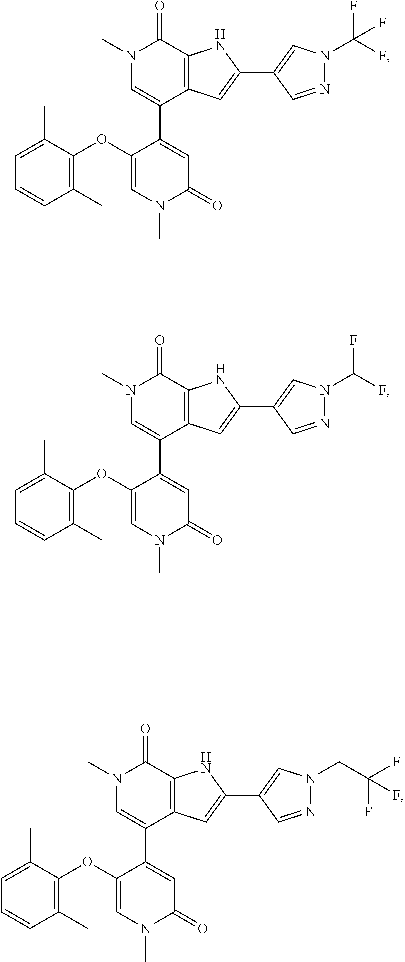 [SMILES: Cc1cccc(C)c1Oc1cn(C)c(=O)cc1-c1cn(C)c(=O)c2[nH]c(-c3cnn(C(F)(F)F)c3)cc12 ; Cc1cccc(C)c1Oc1cn(C)c(=O)cc1-c1cn(C)c(=O)c2[nH]c(-c3cnn(C(F)F)c3)cc12 ; Cc1cccc(C)c1Oc1cn(C)c(=O)cc1-c1cn(C)c(=O)c2[nH]c(-c3cnn(CC(F)(F)F)c3)cc12]

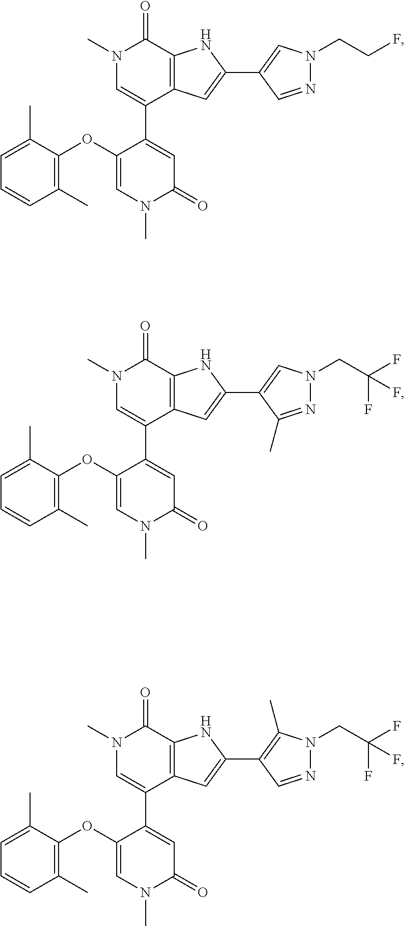 [SMILES: Cc1cccc(C)c1Oc1cn(C)c(=O)cc1-c1cn(C)c(=O)c2[nH]c(-c3cnn(CCF)c3)cc12 ; Cc1cccc(C)c1Oc1cn(C)c(=O)cc1-c1cn(C)c(=O)c2[nH]c(-c3cn(CC(F)(F)F)nc3C)cc12 ; Cc1cccc(C)c1Oc1cn(C)c(=O)cc1-c1cn(C)c(=O)c2[nH]c(-c3cnn(CC(F)(F)F)c3C)cc12]

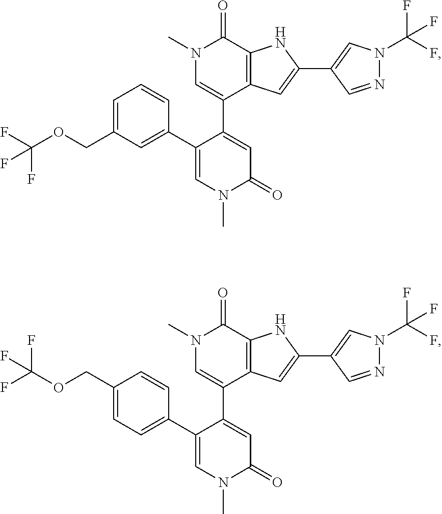 [SMILES: Cn1cc(-c2cccc(COC(F)(F)F)c2)c(-c2cn(C)c(=O)c3[nH]c(-c4cnn(C(F)(F)F)c4)cc23)cc1=O ; Cn1cc(-c2ccc(COC(F)(F)F)cc2)c(-c2cn(C)c(=O)c3[nH]c(-c4cnn(C(F)(F)F)c4)cc23)cc1=O]

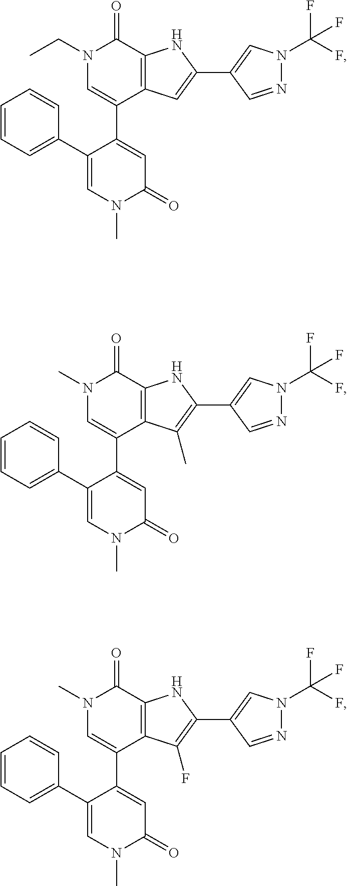 [SMILES: CCn1cc(-c2cc(=O)n(C)cc2-c2ccccc2)c2cc(-c3cnn(C(F)(F)F)c3)[nH]c2c1=O ; Cc1c(-c2cnn(C(F)(F)F)c2)[nH]c2c(=O)n(C)cc(-c3cc(=O)n(C)cc3-c3ccccc3)c12 ; Cn1cc(-c2ccccc2)c(-c2cn(C)c(=O)c3[nH]c(-c4cnn(C(F)(F)F)c4)c(F)c23)cc1=O]

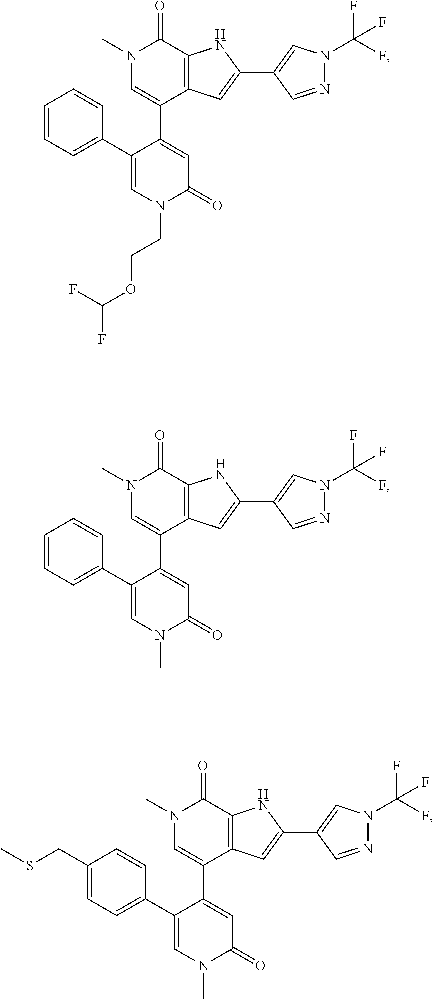 [SMILES: Cn1cc(-c2cc(=O)n(CCOC(F)F)cc2-c2ccccc2)c2cc(-c3cnn(C(F)(F)F)c3)[nH]c2c1=O ; Cn1cc(-c2ccccc2)c(-c2cn(C)c(=O)c3[nH]c(-c4cnn(C(F)(F)F)c4)cc23)cc1=O ; CSCc1ccc(-c2cn(C)c(=O)cc2-c2cn(C)c(=O)c3[nH]c(-c4cnn(C(F)(F)F)c4)cc23)cc1]

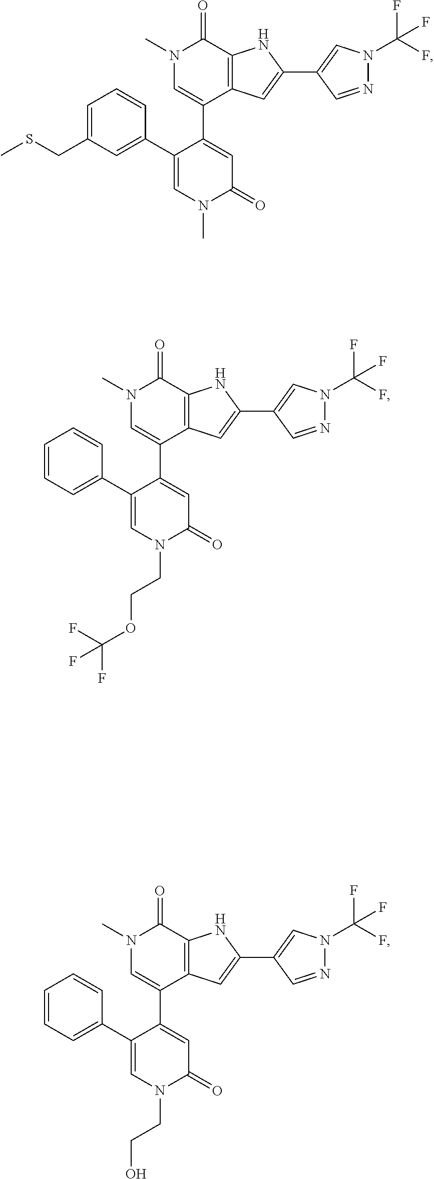 [SMILES: CSCc1cccc(-c2cn(C)c(=O)cc2-c2cn(C)c(=O)c3[nH]c(-c4cnn(C(F)(F)F)c4)cc23)c1 ; Cn1cc(-c2cc(=O)n(CCOC(F)(F)F)cc2-c2ccccc2)c2cc(-c3cnn(C(F)(F)F)c3)[nH]c2c1=O ; Cn1cc(-c2cc(=O)n(CCO)cc2-c2ccccc2)c2cc(-c3cnn(C(F)(F)F)c3)[nH]c2c1=O]

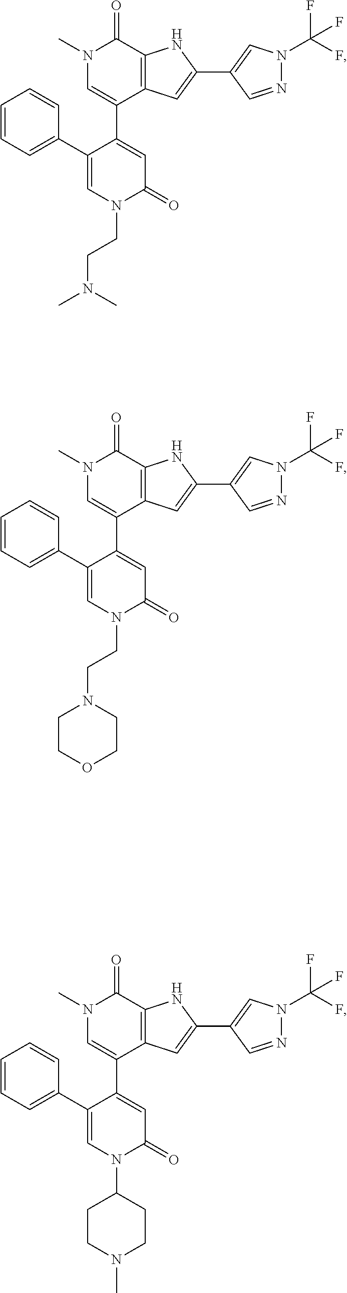 [SMILES: CN(C)CCn1cc(-c2ccccc2)c(-c2cn(C)c(=O)c3[nH]c(-c4cnn(C(F)(F)F)c4)cc23)cc1=O ; Cn1cc(-c2cc(=O)n(CCN3CCOCC3)cc2-c2ccccc2)c2cc(-c3cnn(C(F)(F)F)c3)[nH]c2c1=O ; CN1CCC(n2cc(-c3ccccc3)c(-c3cn(C)c(=O)c4[nH]c(-c5cnn(C(F)(F)F)c5)cc34)cc2=O)CC1]

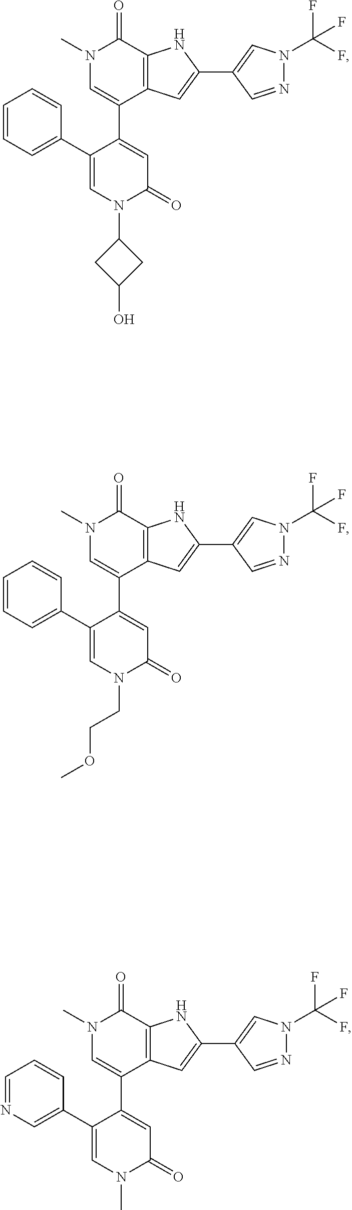 [SMILES: Cn1cc(-c2cc(=O)n(C3CC(O)C3)cc2-c2ccccc2)c2cc(-c3cnn(C(F)(F)F)c3)[nH]c2c1=O ; COCCn1cc(-c2ccccc2)c(-c2cn(C)c(=O)c3[nH]c(-c4cnn(C(F)(F)F)c4)cc23)cc1=O ; Cn1cc(-c2cccnc2)c(-c2cn(C)c(=O)c3[nH]c(-c4cnn(C(F)(F)F)c4)cc23)cc1=O]

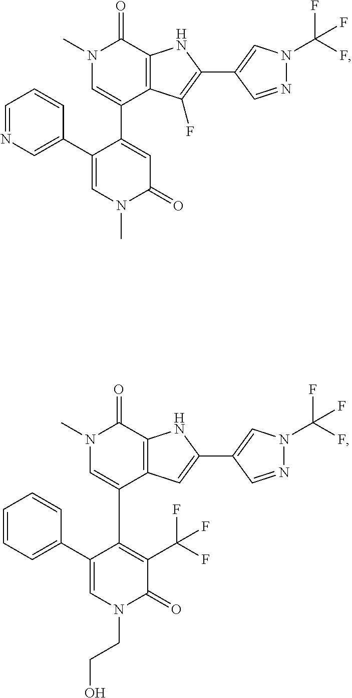 [SMILES: Cn1cc(-c2cccnc2)c(-c2cn(C)c(=O)c3[nH]c(-c4cnn(C(F)(F)F)c4)c(F)c23)cc1=O ; Cn1cc(-c2c(-c3ccccc3)cn(CCO)c(=O)c2C(F)(F)F)c2cc(-c3cnn(C(F)(F)F)c3)[nH]c2c1=O]

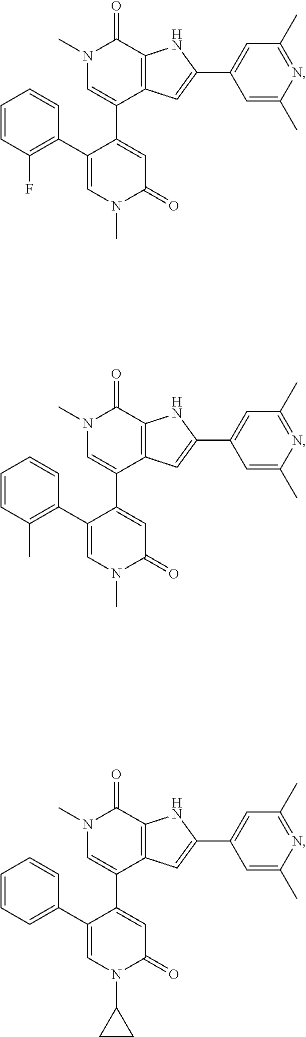 [SMILES: Cc1cc(-c2cc3c(-c4cc(=O)n(C)cc4-c4ccccc4F)cn(C)c(=O)c3[nH]2)cc(C)n1 ; Cc1cc(-c2cc3c(-c4cc(=O)n(C)cc4-c4ccccc4C)cn(C)c(=O)c3[nH]2)cc(C)n1 ; Cc1cc(-c2cc3c(-c4cc(=O)n(C5CC5)cc4-c4ccccc4)cn(C)c(=O)c3[nH]2)cc(C)n1]

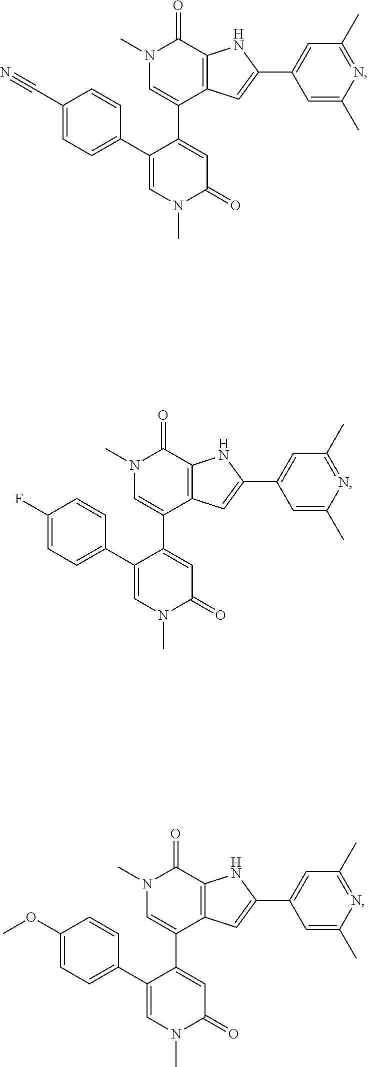

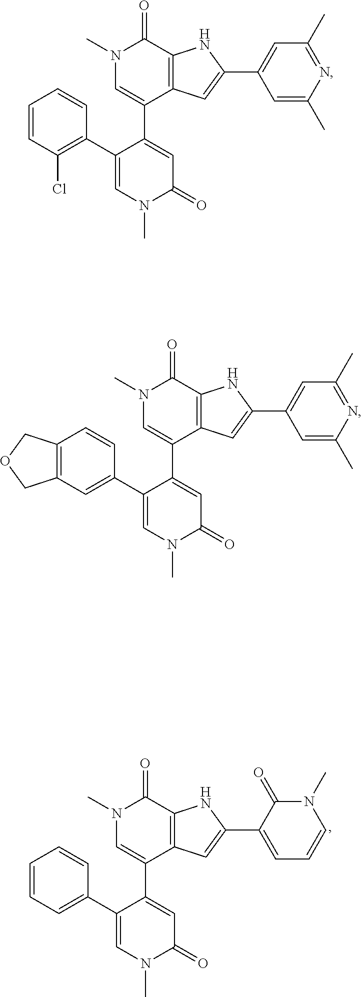 [SMILES: Cc1cc(-c2cc3c(-c4cc(=O)n(C)cc4-c4ccccc4Cl)cn(C)c(=O)c3[nH]2)cc(C)n1 ; Cc1cc(-c2cc3c(-c4cc(=O)n(C)cc4-c4ccc5c(c4)COC5)cn(C)c(=O)c3[nH]2)cc(C)n1 ; Cn1cc(-c2ccccc2)c(-c2cn(C)c(=O)c3[nH]c(-c4cccn(C)c4=O)cc23)cc1=O]

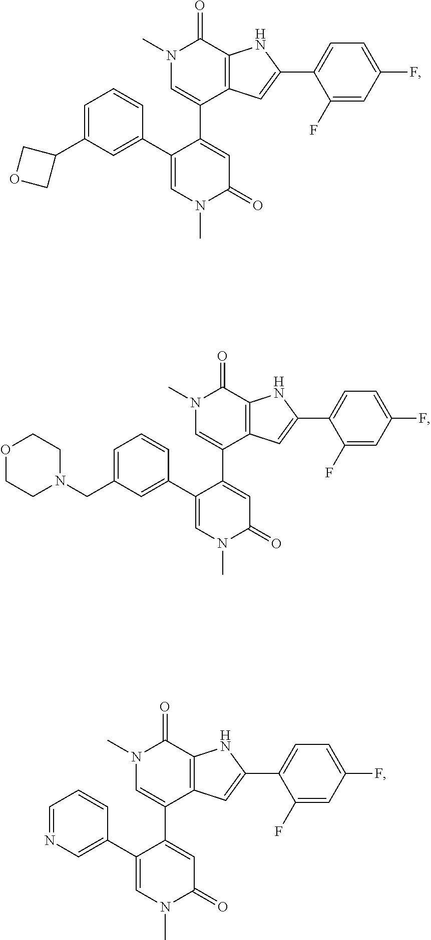 [SMILES: Cn1cc(-c2cccc(C3COC3)c2)c(-c2cn(C)c(=O)c3[nH]c(-c4ccc(F)cc4F)cc23)cc1=O ; Cn1cc(-c2cccc(CN3CCOCC3)c2)c(-c2cn(C)c(=O)c3[nH]c(-c4ccc(F)cc4F)cc23)cc1=O ; Cn1cc(-c2cccnc2)c(-c2cn(C)c(=O)c3[nH]c(-c4ccc(F)cc4F)cc23)cc1=O]

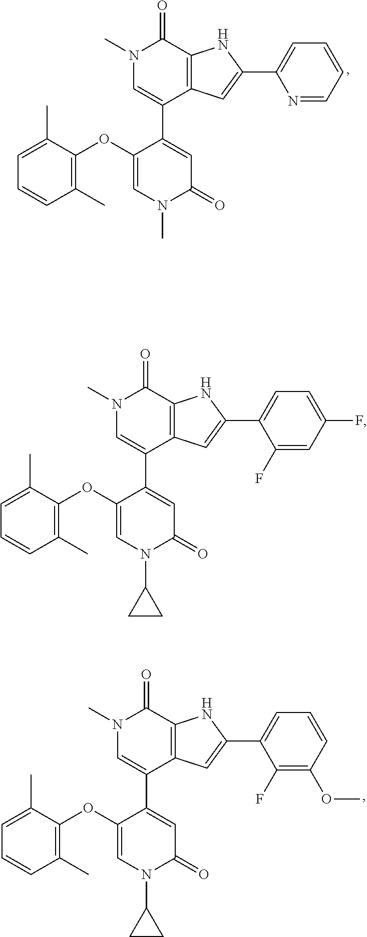 [SMILES: Cc1cccc(C)c1Oc1cn(C)c(=O)cc1-c1cn(C)c(=O)c2[nH]c(-c3ccccn3)cc12 ; Cc1cccc(C)c1Oc1cn(C2CC2)c(=O)cc1-c1cn(C)c(=O)c2[nH]c(-c3ccc(F)cc3F)cc12 ; COc1cccc(-c2cc3c(-c4cc(=O)n(C5CC5)cc4Oc4c(C)cccc4C)cn(C)c(=O)c3[nH]2)c1F]

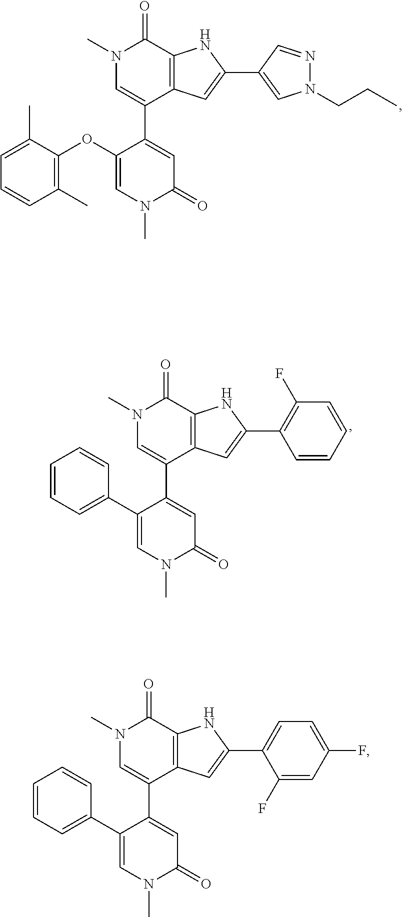 [SMILES: CCCn1cc(-c2cc3c(-c4cc(=O)n(C)cc4Oc4c(C)cccc4C)cn(C)c(=O)c3[nH]2)cn1 ; Cn1cc(-c2ccccc2)c(-c2cn(C)c(=O)c3[nH]c(-c4ccccc4F)cc23)cc1=O ; Cn1cc(-c2ccccc2)c(-c2cn(C)c(=O)c3[nH]c(-c4ccc(F)cc4F)cc23)cc1=O]

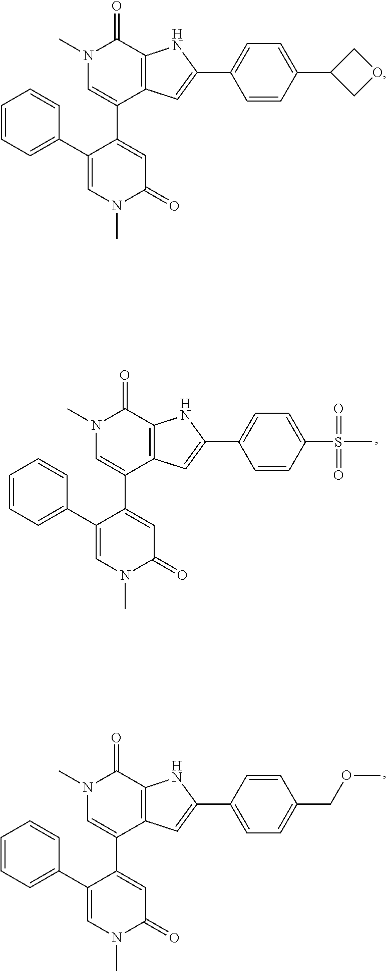 [SMILES: Cn1cc(-c2ccccc2)c(-c2cn(C)c(=O)c3[nH]c(-c4ccc(C5COC5)cc4)cc23)cc1=O ; Cn1cc(-c2ccccc2)c(-c2cn(C)c(=O)c3[nH]c(-c4ccc(S(C)(=O)=O)cc4)cc23)cc1=O ; COCc1ccc(-c2cc3c(-c4cc(=O)n(C)cc4-c4ccccc4)cn(C)c(=O)c3[nH]2)cc1]

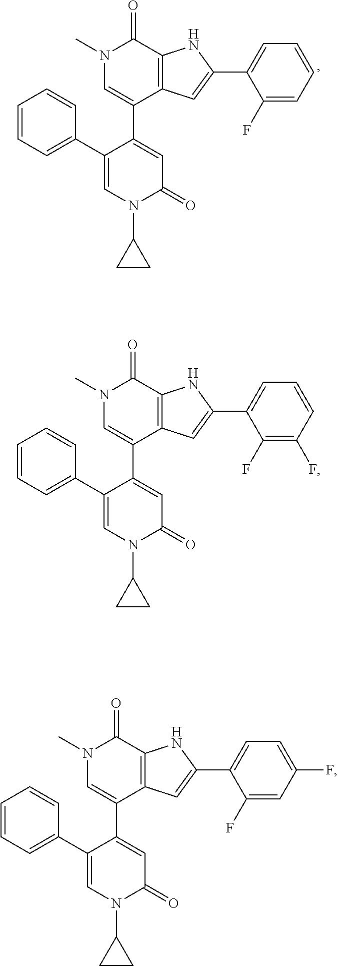 [SMILES: Cn1cc(-c2cc(=O)n(C3CC3)cc2-c2ccccc2)c2cc(-c3ccccc3F)[nH]c2c1=O ; Cn1cc(-c2cc(=O)n(C3CC3)cc2-c2ccccc2)c2cc(-c3cccc(F)c3F)[nH]c2c1=O ; Cn1cc(-c2cc(=O)n(C3CC3)cc2-c2ccccc2)c2cc(-c3ccc(F)cc3F)[nH]c2c1=O]

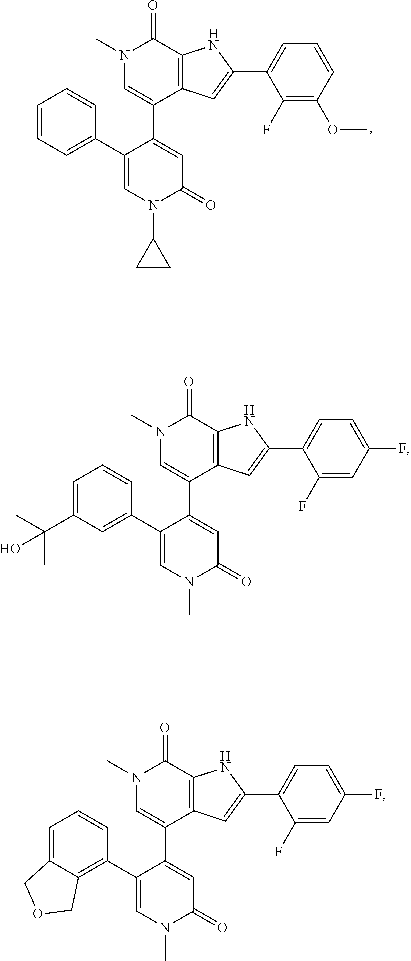

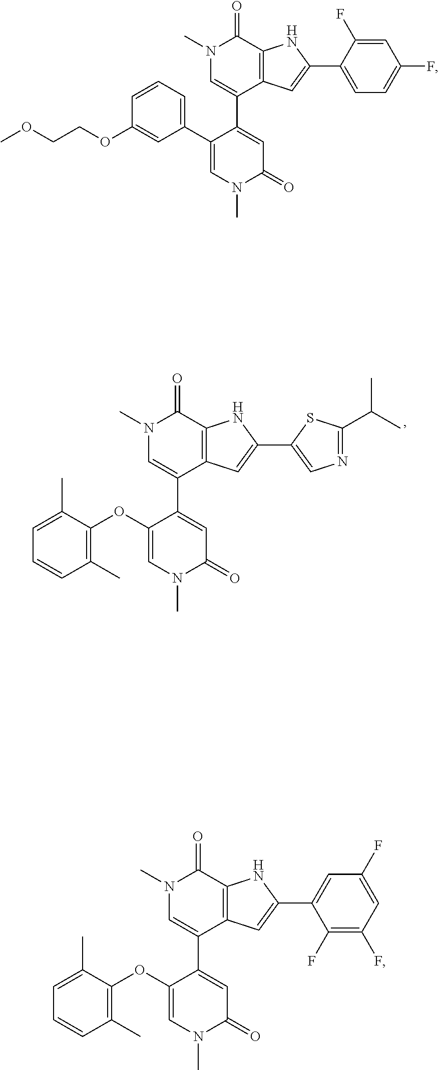 [SMILES: COCCOc1cccc(-c2cn(C)c(=O)cc2-c2cn(C)c(=O)c3[nH]c(-c4ccc(F)cc4F)cc23)c1 ; Cc1cccc(C)c1Oc1cn(C)c(=O)cc1-c1cn(C)c(=O)c2[nH]c(-c3cnc(C(C)C)s3)cc12 ; Cc1cccc(C)c1Oc1cn(C)c(=O)cc1-c1cn(C)c(=O)c2[nH]c(-c3cc(F)cc(F)c3F)cc12]

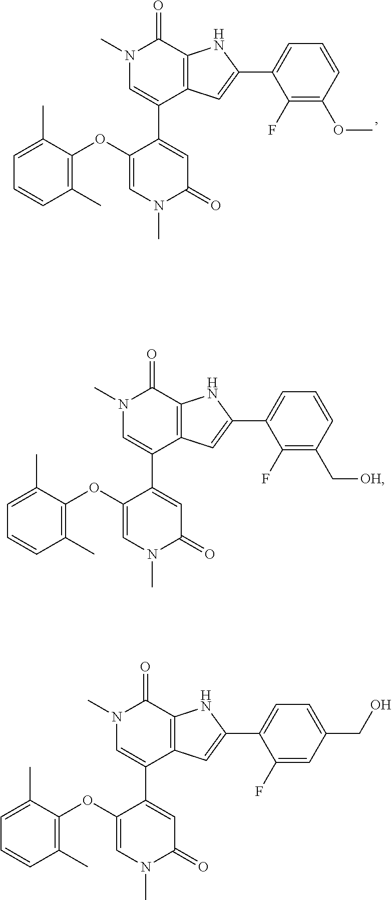 [SMILES: COc1cccc(-c2cc3c(-c4cc(=O)n(C)cc4Oc4c(C)cccc4C)cn(C)c(=O)c3[nH]2)c1F ; Cc1cccc(C)c1Oc1cn(C)c(=O)cc1-c1cn(C)c(=O)c2[nH]c(-c3cccc(CO)c3F)cc12 ; Cc1cccc(C)c1Oc1cn(C)c(=O)cc1-c1cn(C)c(=O)c2[nH]c(-c3ccc(CO)cc3F)cc12]

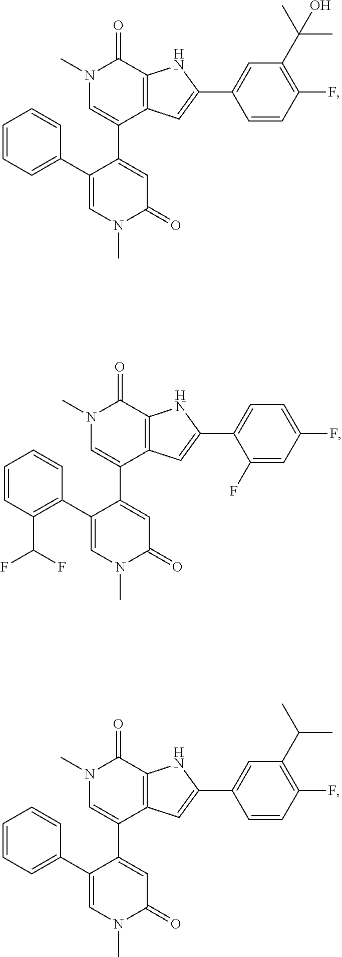 [SMILES: Cn1cc(-c2ccccc2)c(-c2cn(C)c(=O)c3[nH]c(-c4ccc(F)c(C(C)(C)O)c4)cc23)cc1=O ; Cn1cc(-c2ccccc2C(F)F)c(-c2cn(C)c(=O)c3[nH]c(-c4ccc(F)cc4F)cc23)cc1=O ; CC(C)c1cc(-c2cc3c(-c4cc(=O)n(C)cc4-c4ccccc4)cn(C)c(=O)c3[nH]2)ccc1F]

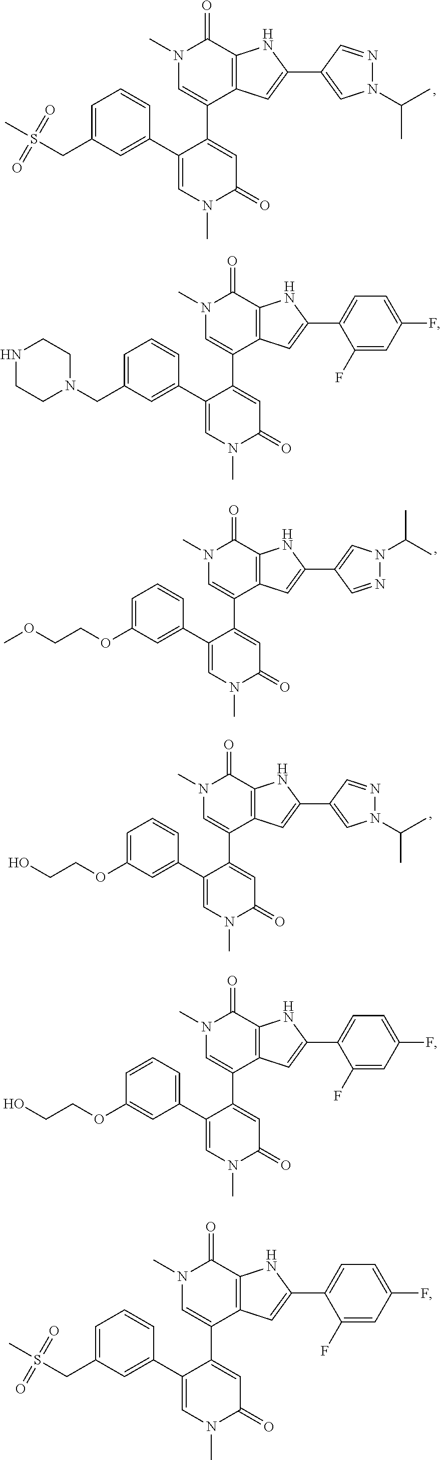

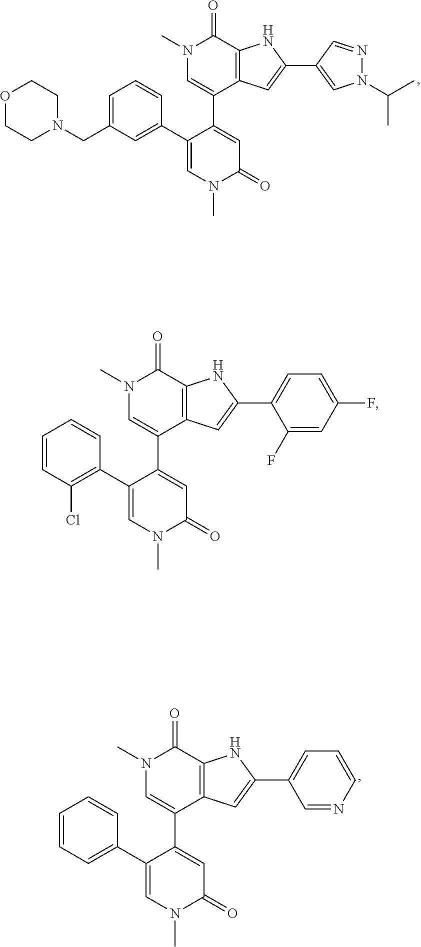 [SMILES: CC(C)n1cc(-c2cc3c(-c4cc(=O)n(C)cc4-c4cccc(CN5CCOCC5)c4)cn(C)c(=O)c3[nH]2)cn1 ; Cn1cc(-c2ccccc2Cl)c(-c2cn(C)c(=O)c3[nH]c(-c4ccc(F)cc4F)cc23)cc1=O ; Cn1cc(-c2ccccc2)c(-c2cn(C)c(=O)c3[nH]c(-c4cccnc4)cc23)cc1=O]

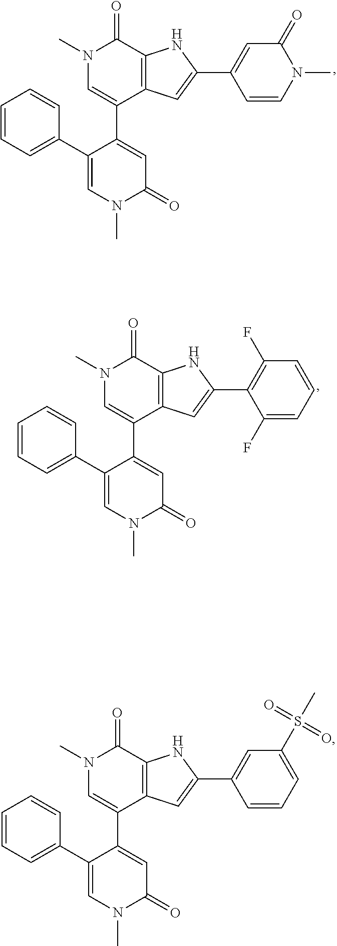 [SMILES: Cn1ccc(-c2cc3c(-c4cc(=O)n(C)cc4-c4ccccc4)cn(C)c(=O)c3[nH]2)cc1=O ; Cn1cc(-c2ccccc2)c(-c2cn(C)c(=O)c3[nH]c(-c4c(F)cccc4F)cc23)cc1=O ; Cn1cc(-c2ccccc2)c(-c2cn(C)c(=O)c3[nH]c(-c4cccc(S(C)(=O)=O)c4)cc23)cc1=O]

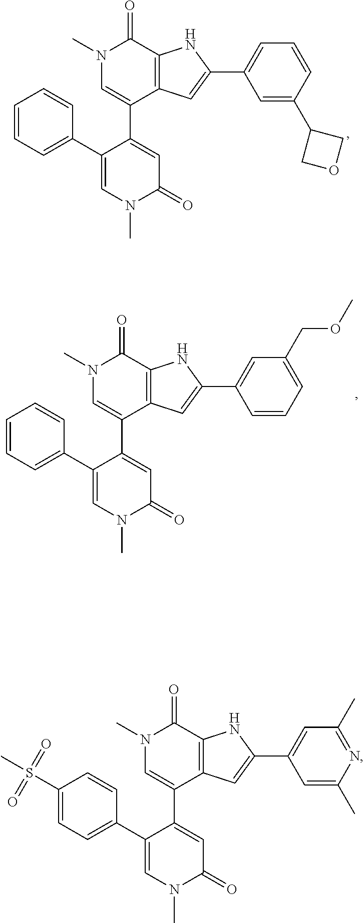

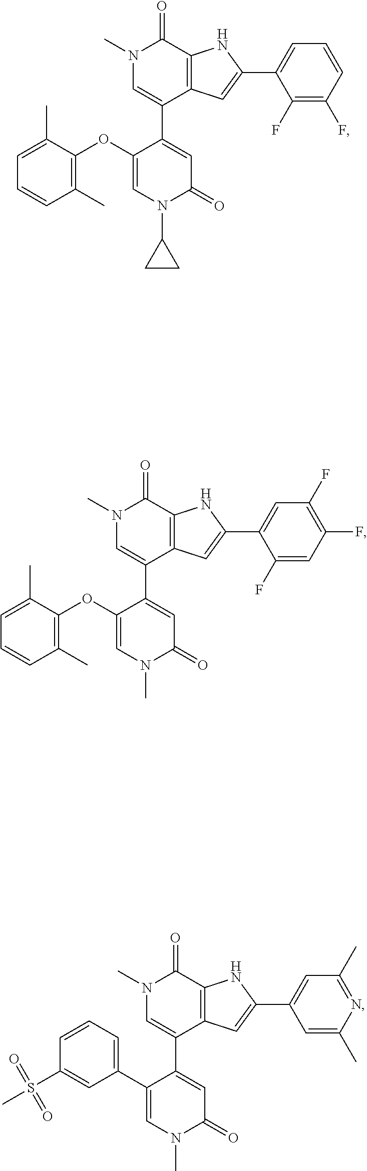

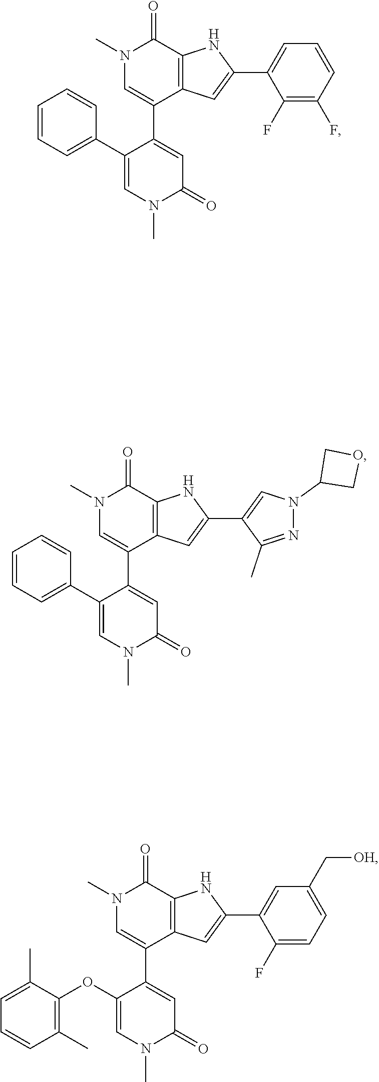 [SMILES: Cn1cc(-c2ccccc2)c(-c2cn(C)c(=O)c3[nH]c(-c4cccc(F)c4F)cc23)cc1=O ; Cc1nn(C2COC2)cc1-c1cc2c(-c3cc(=O)n(C)cc3-c3ccccc3)cn(C)c(=O)c2[nH]1 ; Cc1cccc(C)c1Oc1cn(C)c(=O)cc1-c1cn(C)c(=O)c2[nH]c(-c3cc(CO)ccc3F)cc12]

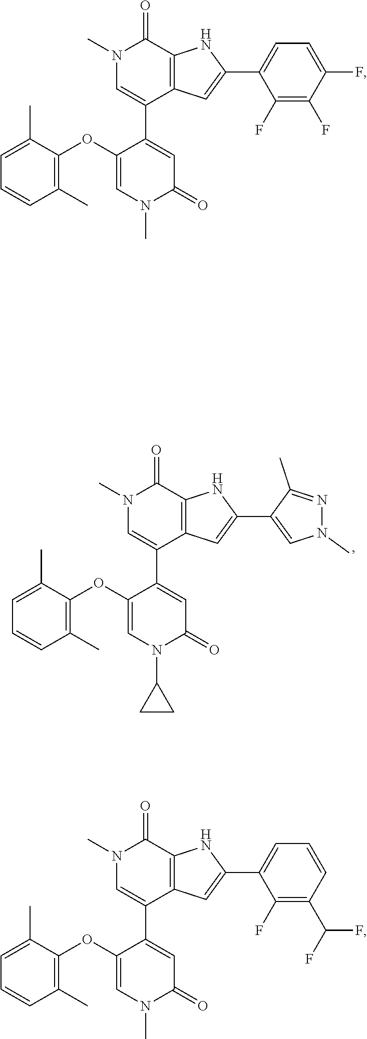 [SMILES: Cc1cccc(C)c1Oc1cn(C)c(=O)cc1-c1cn(C)c(=O)c2[nH]c(-c3ccc(F)c(F)c3F)cc12 ; Cc1cccc(C)c1Oc1cn(C2CC2)c(=O)cc1-c1cn(C)c(=O)c2[nH]c(-c3cn(C)nc3C)cc12 ; Cc1cccc(C)c1Oc1cn(C)c(=O)cc1-c1cn(C)c(=O)c2[nH]c(-c3cccc(C(F)F)c3F)cc12]

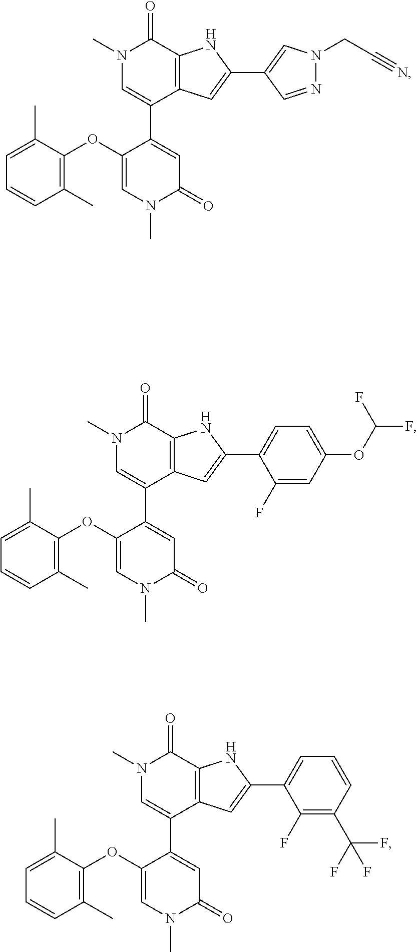 [SMILES: Cc1cccc(C)c1Oc1cn(C)c(=O)cc1-c1cn(C)c(=O)c2[nH]c(-c3cnn(CC#N)c3)cc12 ; Cc1cccc(C)c1Oc1cn(C)c(=O)cc1-c1cn(C)c(=O)c2[nH]c(-c3ccc(OC(F)F)cc3F)cc12 ; Cc1cccc(C)c1Oc1cn(C)c(=O)cc1-c1cn(C)c(=O)c2[nH]c(-c3cccc(C(F)(F)F)c3F)cc12]

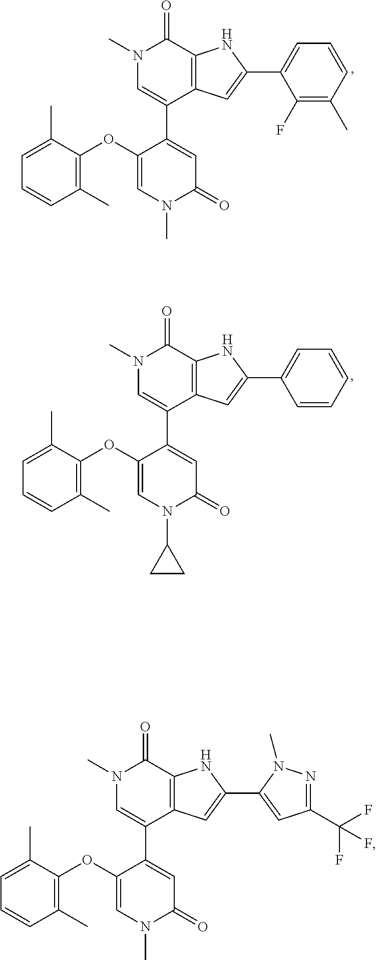 [SMILES: Cc1cccc(-c2cc3c(-c4cc(=O)n(C)cc4Oc4c(C)cccc4C)cn(C)c(=O)c3[nH]2)c1F ; Cc1cccc(C)c1Oc1cn(C2CC2)c(=O)cc1-c1cn(C)c(=O)c2[nH]c(-c3ccccc3)cc12 ; Cc1cccc(C)c1Oc1cn(C)c(=O)cc1-c1cn(C)c(=O)c2[nH]c(-c3cc(C(F)(F)F)nn3C)cc12]

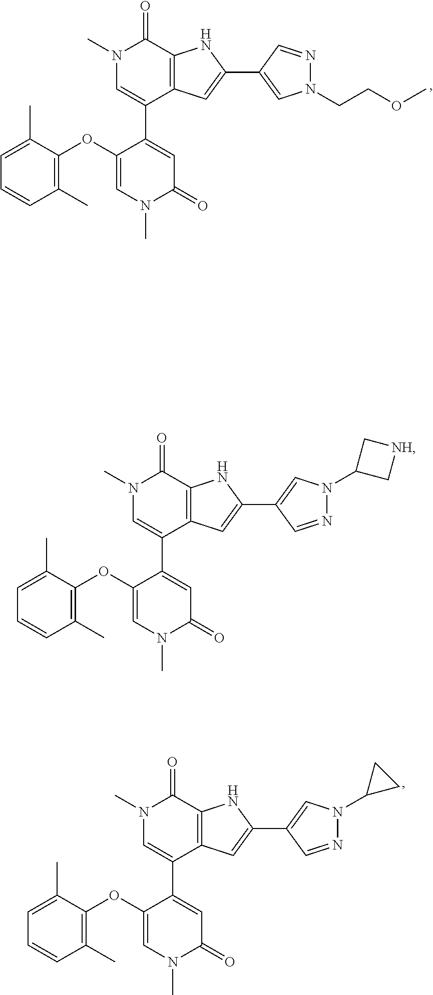 [SMILES: COCCn1cc(-c2cc3c(-c4cc(=O)n(C)cc4Oc4c(C)cccc4C)cn(C)c(=O)c3[nH]2)cn1 ; Cc1cccc(C)c1Oc1cn(C)c(=O)cc1-c1cn(C)c(=O)c2[nH]c(-c3cnn(C4CNC4)c3)cc12 ; Cc1cccc(C)c1Oc1cn(C)c(=O)cc1-c1cn(C)c(=O)c2[nH]c(-c3cnn(C4CC4)c3)cc12]

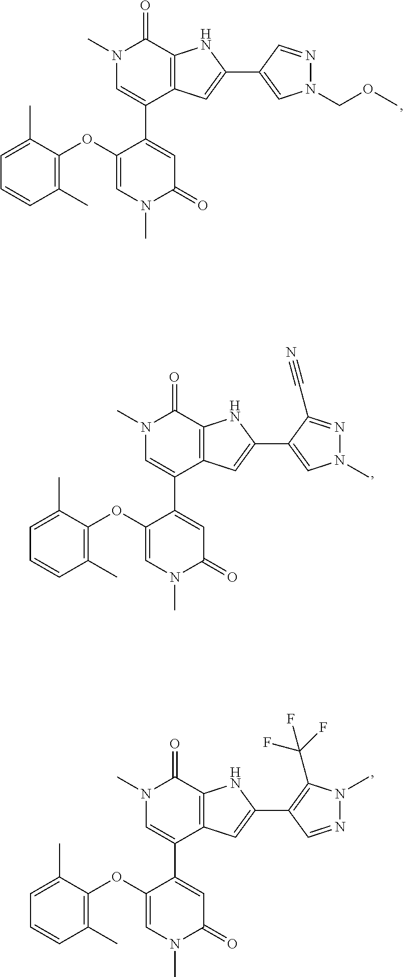 [SMILES: COCn1cc(-c2cc3c(-c4cc(=O)n(C)cc4Oc4c(C)cccc4C)cn(C)c(=O)c3[nH]2)cn1 ; Cc1cccc(C)c1Oc1cn(C)c(=O)cc1-c1cn(C)c(=O)c2[nH]c(-c3cn(C)nc3C#N)cc12 ; Cc1cccc(C)c1Oc1cn(C)c(=O)cc1-c1cn(C)c(=O)c2[nH]c(-c3cnn(C)c3C(F)(F)F)cc12]

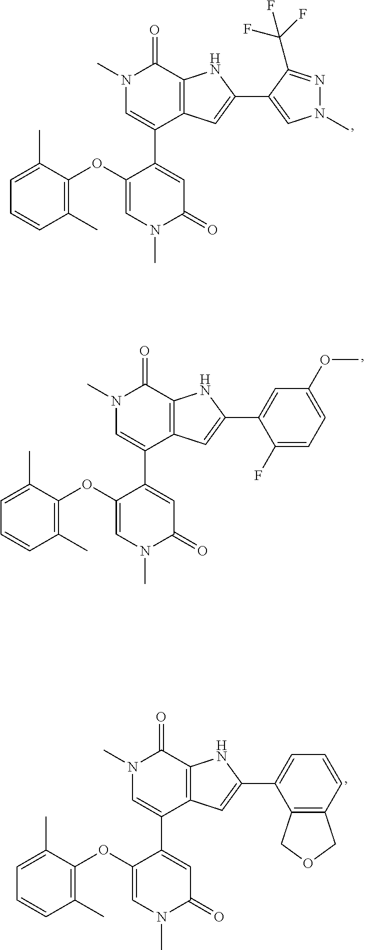 [SMILES: Cc1cccc(C)c1Oc1cn(C)c(=O)cc1-c1cn(C)c(=O)c2[nH]c(-c3cn(C)nc3C(F)(F)F)cc12 ; COc1ccc(F)c(-c2cc3c(-c4cc(=O)n(C)cc4Oc4c(C)cccc4C)cn(C)c(=O)c3[nH]2)c1 ; Cc1cccc(C)c1Oc1cn(C)c(=O)cc1-c1cn(C)c(=O)c2[nH]c(-c3cccc4c3COC4)cc12]

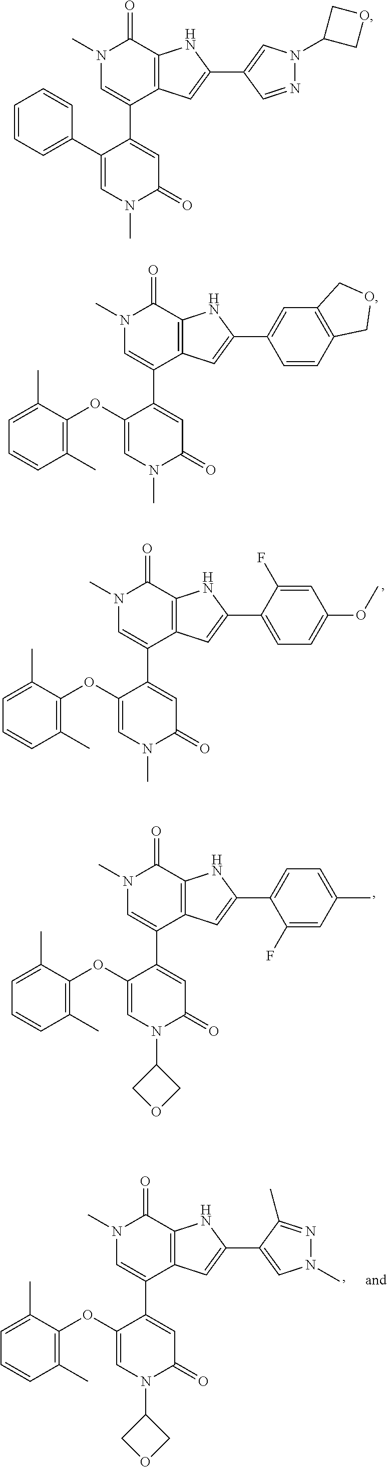

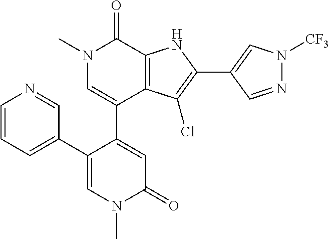 [SMILES: Cn1cc(-c2cccnc2)c(-c2cn(C)c(=O)c3[nH]c(-c4cnn(C(F)(F)F)c4)c(Cl)c23)cc1=O]

or a stereoisomer, or a pharmaceutically acceptable salt or N-oxide thereof.
41. A pharmaceutical composition comprising a compound of any one of the preceding embodiments, and one or more pharmaceutically acceptable excipients.
42. A compound of any one of the preceding embodiments for use as a medicament.
43. A compound of any one of the preceding embodiments for use in treatment or prophylaxis of an inflammatory disorder or disease.
44. A compound of any one of the preceding embodiments for use in treatment or prophylaxis of an immune disorder.
45. A compound of any one of the preceding embodiments for use in treatment or prophylaxis of an autoimmune disease.
46. A compound of any one of the preceding embodiments for use in treatment of cancer.
47. A compound of any one of the preceding embodiments, wherein the use comprises topical or oral administration of the compound.
48. A compound of any one of the preceding embodiments, wherein the use comprises administration by injection of the compound.
49 A compound of any one of the preceding embodiments, wherein the use comprises topical administration of the compound to the skin.

This disclosure additionally includes the following numbered embodiments:
1. A method for the treatment of an inflammatory and/or an autoimmune disease or disorder or a disease or disorder related thereto, the method comprising administering to a subject in need thereof a therapeutically effective amount of a compound of formula (XXIA), a tautomer, a stereoisomer or a mixture of stereoisomers, a pharmaceutically acceptable salt, a hydrate, a deuterated derivative, or an N-oxide thereof:

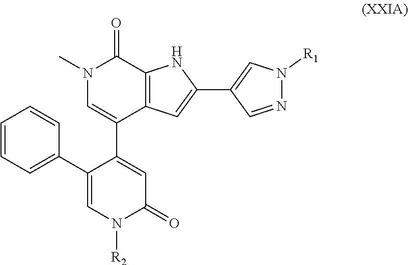

wherein:
R₁ is C₁-C₄-haloalkyl;
R₂ is C₁-C₄-alkyl, wherein the C₁-C₄-alkyl is optionally substituted with SR₃ or OR₃; and
R₃ is selected from H, CI—C₃-alkyl, C(O)—C₁-C₃-alkyl, and C₁-C₃-haloalkyl.
2. The method of any one of the preceding embodiments, wherein R₁ is independently selected from CF₃, CHF₂, CH₂CF₃, and CH₂CH₂F.
3. The method of any one of the preceding embodiments, wherein R₁ is independently selected from CBr₃, CHBr₂, CH₂CBr₃, and CH₂CH₂Br.
4. The method of any one of the preceding embodiments, wherein R₁ is independently selected from CCl₃, CHCl₂, CH₂CCl₃, and CH₂CH₂Cl.
5. The method of any one of the preceding embodiments, wherein R₁ is CH₂CH₂F.
6. The method of any one of the preceding embodiments, wherein R₁ is CHF₂.
7. The method of any one of the preceding embodiments, wherein R₁ is CF₃.
8. The method of any one of the preceding embodiments, wherein R₂ is C₁-C₄-alkyl, wherein the C₁-C₄-alkyl is substituted with OR₃.
9. The method of any one of the preceding embodiments, wherein R₂ is C₁-C₄-alkyl, wherein the C₁-C₄-alkyl is substituted with SR₃.
10. The method of any one of the preceding embodiments, wherein R₂ is selected from —CH₃, —CH₂—CH₂—O—CH₃, and —CH₂—CH₂—OCH₂—CH₃.
11. The method of any one of the preceding embodiments, wherein R₂ is selected from —CH₃, —CH₂—CH₂—S—CH₃, and —CH₂—CH₂—SCH₂—CH₃.
12. The method of any one of the preceding embodiments, wherein R₂ is —CH₃.
13. The method of any one of the preceding embodiments, wherein R₂ is —CH₂—CH₂—O—CH₃.
14. The method of any one of the preceding embodiments, wherein R₂ is —CH₂—CH₂—S—CH₃.
15. The method of any one of the preceding embodiments, wherein the compound of formula (XXIA) is selected from:

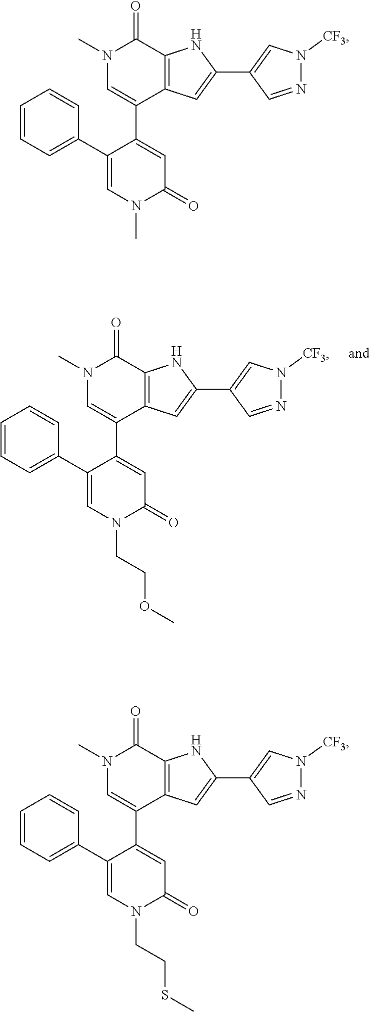 [SMILES: Cn1cc(-c2ccccc2)c(-c2cn(C)c(=O)c3[nH]c(-c4cnn(C(F)(F)F)c4)cc23)cc1=O ; COCCn1cc(-c2ccccc2)c(-c2cn(C)c(=O)c3[nH]c(-c4cnn(C(F)(F)F)c4)cc23)cc1=O ; "and" ; CSCCn1cc(-c2ccccc2)c(-c2cn(C)c(=O)c3[nH]c(-c4cnn(C(F)(F)F)c4)cc23)cc1=O]

or a tautomer, a stereoisomer or mixture of stereoisomers, a pharmaceutically acceptable salt, a hydrate, a deuterated derivative, or N-oxide thereof.
16. The method of any one of the preceding embodiments, wherein the compound of formula (XXIA) is

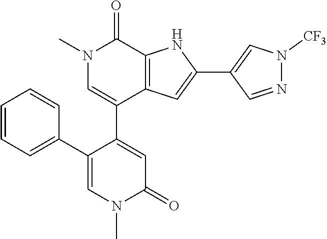 [SMILES: Cn1cc(-c2ccccc2)c(-c2cn(C)c(=O)c3[nH]c(-c4cnn(C(F)(F)F)c4)cc23)cc1=O]

or a tautomer, a stereoisomer or mixture of stereoisomers, a pharmaceutically acceptable salt, a hydrate, a deuterated derivative, or N-oxide thereof.
17. The method of any one of the preceding embodiments, wherein the compound of formula (XXIA) is

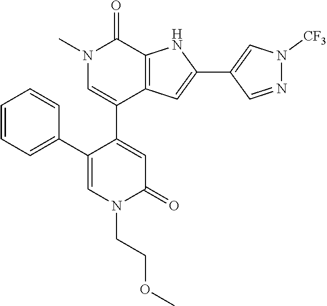 [SMILES: COCCn1cc(-c2ccccc2)c(-c2cn(C)c(=O)c3[nH]c(-c4cnn(C(F)(F)F)c4)cc23)cc1=O]

or a tautomer, a stereoisomer or mixture of stereoisomers, a pharmaceutically acceptable salt, a hydrate, a deuterated derivative, or N-oxide thereof.
18. The method of any one of the preceding embodiments, wherein the compound of formula (XXIA) is

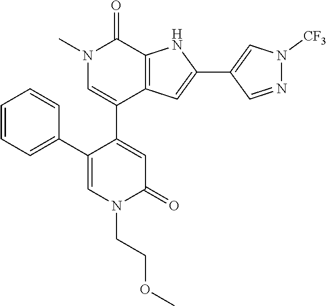 [SMILES: COCCn1cc(-c2ccccc2)c(-c2cn(C)c(=O)c3[nH]c(-c4cnn(C(F)(F)F)c4)cc23)cc1=O]

or a tautomer, a stereoisomer or mixture of stereoisomers, a pharmaceutically acceptable salt, a hydrate, a deuterated derivative, or N-oxide thereof.
19. The method of any one of the preceding embodiments, wherein the disease or disorder is a joint or joint-related disorder.
20. The method of any one of the preceding embodiments, wherein the joint related disease or disorder is chosen from arthritis, bursitis, Ehlers-Danlos syndrome, epicondylitis, Felty Syndrome, gouty arthritis, psoriatic arthritis, osteoarthritis, rheumatoid arthritis, Still's disease, tenosynovitis, synovitis, Sjögren's Syndrome, Lyme disease, Whipple disease, bone cancer, lupus, and other autoimmune joint disorders.
21. The method of any one of the preceding embodiments, wherein the joint or joint related disease or disorder comprises an arthritis.
22. The method of any one of the preceding embodiments, wherein the arthritis comprises rheumatoid arthritis.
23. The method of any one of the preceding embodiments, wherein the disorder is an arthritis and upon administration of the therapeutically effective amount of the compound, tautomer, stereoisomer or mixture of stereoisomers, pharmaceutically acceptable salt, hydrate, deuterated derivative, or N-oxide thereof there is a therapeutic effect associated with reduction in inflammation.
24. The method of any one of the preceding embodiments, wherein the therapeutic effect associated with a reduction in inflammation is a reduction in thickness or girth of a joint or limb.
25. The method of any one of the preceding embodiments, wherein there is reduction in arthritic scoring or severity, and wherein the reduction in arthritic scoring or severity is a reduction in:
   (a) definite redness and swelling of an ankle/wrist or apparent redness and swelling limited to individual digits, regardless of the number of affected digits;
   (b) severe redness and swelling of an ankle/wrist;
   (c) redness and swelling of an entire appendage including digits; and/or
   (d) maximally inflamed limb with involvement of multiple joints.
26. The method of any one of the preceding embodiments, wherein the reduction is dose dependent.
27. The method of any one of the preceding embodiments, wherein the reduction is by >about 50%.
28. A method for the treatment of a joint or joint-related disease, the method comprising orally administering to a subject in need thereof a therapeutically effective amount of a compound that is selected from:

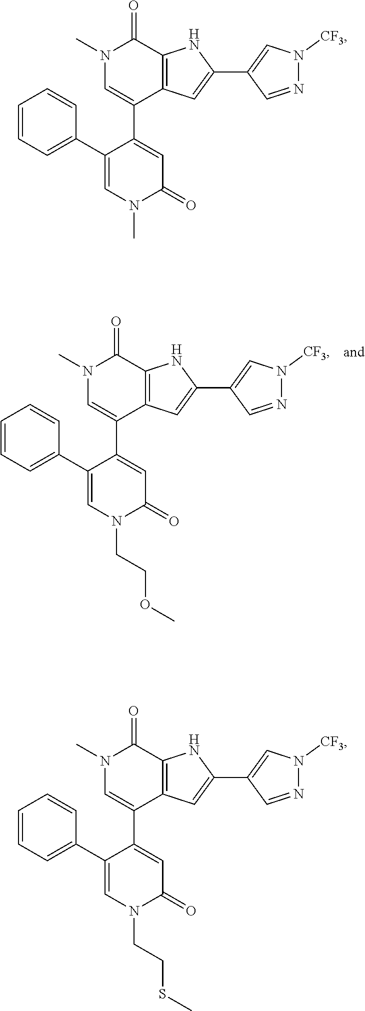 [SMILES: Cn1cc(-c2ccccc2)c(-c2cn(C)c(=O)c3[nH]c(-c4cnn(C(F)(F)F)c4)cc23)cc1=O ; COCCn1cc(-c2ccccc2)c(-c2cn(C)c(=O)c3[nH]c(-c4cnn(C(F)(F)F)c4)cc23)cc1=O ; CSCCn1cc(-c2ccccc2)c(-c2cn(C)c(=O)c3[nH]c(-c4cnn(C(F)(F)F)c4)cc23)cc1=O]

or a tautomer, stereoisomer or mixture of stereoisomers, pharmaceutically acceptable salt, hydrate, deuterated derivative, or N-oxide thereof.
29. A method for the treatment of a joint or joint-related disease, the method comprising orally administering to a subject in need thereof a therapeutically effective amount of a compound that is

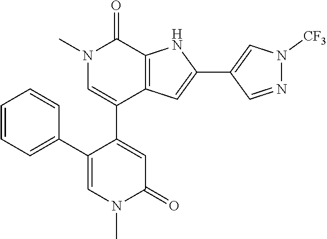 [SMILES: Cn1cc(-c2ccccc2)c(-c2cn(C)c(=O)c3[nH]c(-c4cnn(C(F)(F)F)c4)cc23)cc1=O]

or a tautomer, stereoisomer or mixture of stereoisomers, pharmaceutically acceptable salt, hydrate, deuterated derivative, or N-oxide thereof.
30. A method for the treatment of a joint or joint-related disease, the method comprising orally administering to a subject in need thereof a therapeutically effective amount of a compound that is

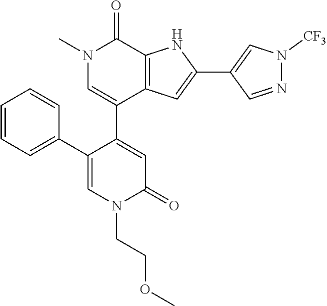 [SMILES: COCCn1cc(-c2ccccc2)c(-c2cn(C)c(=O)c3[nH]c(-c4cnn(C(F)(F)F)c4)cc23)cc1=O]

or a tautomer, stereoisomer or mixture of stereoisomers, pharmaceutically acceptable salt, or hydrate, deuterated derivative, or N-oxide thereof.
31. A method for the treatment of a joint or joint-related disease, the method comprising orally administering to a subject in need thereof a therapeutically effective amount of a compound that is

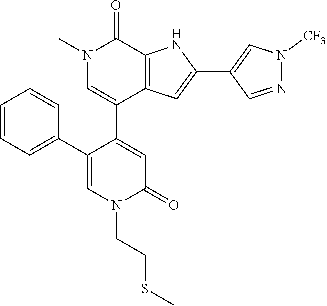 [SMILES: CSCCn1cc(-c2ccccc2)c(-c2cn(C)c(=O)c3[nH]c(-c4cnn(C(F)(F)F)c4)cc23)cc1=O]

or a tautomer, stereoisomer or mixture of stereoisomers, pharmaceutically acceptable salt, hydrate, deuterated derivative, or N-oxide thereof.
32. A method for the treatment of an arthritic disease, the method comprising orally administering to a subject in need thereof a therapeutically effective amount of a compound that is selected from:

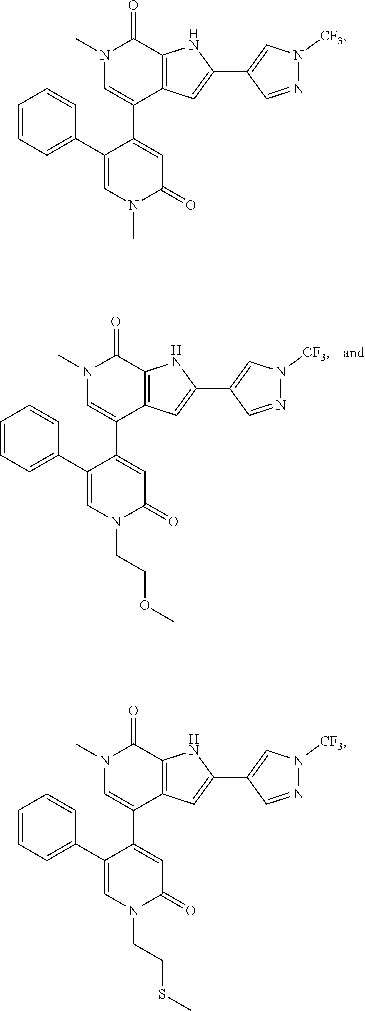 [SMILES: Cn1cc(-c2ccccc2)c(-c2cn(C)c(=O)c3[nH]c(-c4cnn(C(F)(F)F)c4)cc23)cc1=O ; COCCn1cc(-c2ccccc2)c(-c2cn(C)c(=O)c3[nH]c(-c4cnn(C(F)(F)F)c4)cc23)cc1=O ; CSCCn1cc(-c2ccccc2)c(-c2cn(C)c(=O)c3[nH]c(-c4cnn(C(F)(F)F)c4)cc23)cc1=O]

or a tautomer, stereoisomer or mixture of stereoisomers, pharmaceutically acceptable salt, hydrate, deuterated derivative, or N-oxide thereof, wherein upon administration of a therapeutically effective amount of the compound, tautomer, stereoisomer or mixture of stereoisomers, pharmaceutically acceptable salt, hydrate, deuterated derivative, or N-oxide thereof a therapeutic effect is associated with a reduction in inflammation.
33. A method for the treatment of an arthritic disease, the method comprising orally administering to a subject in need thereof a therapeutically effective amount of a compound that is

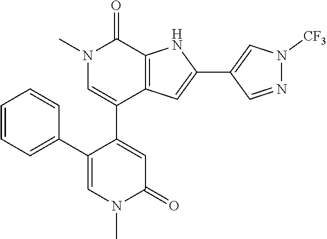 [SMILES: Cn1cc(-c2ccccc2)c(-c2cn(C)c(=O)c3[nH]c(-c4cnn(C(F)(F)F)c4)cc23)cc1=O]

or a tautomer, stereoisomer or mixture of stereoisomers, pharmaceutically acceptable salt, hydrate, deuterated derivative, or N-oxide thereof, wherein upon administration of a therapeutically effective amount of the compound, tautomer, stereoisomer or mixture of stereoisomers, pharmaceutically acceptable salt, hydrate, deuterated derivative, or N-oxide thereof a therapeutic effect is associated with a reduction in inflammation.
34. A method for the treatment of an arthritic disease, the method comprising orally administering to a subject in need thereof a therapeutically effective amount of a compound that is

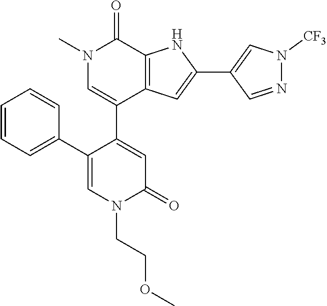 [SMILES: COCCn1cc(-c2ccccc2)c(-c2cn(C)c(=O)c3[nH]c(-c4cnn(C(F)(F)F)c4)cc23)cc1=O]

or a tautomer, stereoisomer or mixture of stereoisomers, pharmaceutically acceptable salt, hydrate, deuterated derivative, or N-oxide thereof, wherein upon administration of a therapeutically effective amount of the compound, tautomer, stereoisomer or mixture of stereoisomers, pharmaceutically acceptable salt, hydrate, deuterated derivative, or N-oxide thereof a therapeutic effect is associated with a reduction in inflammation.
35. A method for the treatment of an arthritic disease, the method comprising orally administering to a subject in need thereof a therapeutically effective amount of a compound that is

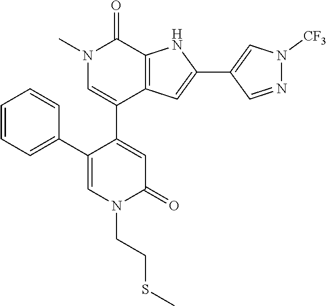 [SMILES: CSCCn1cc(-c2ccccc2)c(-c2cn(C)c(=O)c3[nH]c(-c4cnn(C(F)(F)F)c4)cc23)cc1=O]

or a tautomer, stereoisomer or mixture of stereoisomers, pharmaceutically acceptable salt, hydrate, deuterated derivative, or N-oxide thereof, wherein upon administration of a therapeutically effective amount of the compound, tautomer, stereoisomer or mixture of stereoisomers, pharmaceutically acceptable salt, hydrate, deuterated derivative, or N-oxide thereof a therapeutic effect is associated with a reduction in inflammation.
36. The method of any one of the preceding embodiments, wherein the disease or disorder is a fibrotic disease or disorder.
37. The method of any one of the preceding embodiments, wherein the disease or disorder is renal fibrosis.
38. The method of any one of the preceding embodiments, wherein the disease or disorder is a pulmonary fibrosis (PF).
39. The method of any one of the preceding embodiments, wherein the disease or disorder is idiopathic pulmonary fibrosis (IPF).
40. The method of any one of the preceding embodiments, wherein, upon administration of the therapeutically effective amount of the compound, tautomer, stereoisomer or mixture of stereoisomers, pharmaceutically acceptable salt, hydrate, deuterated derivative, or N-oxide thereof, there is a therapeutic effect associated with a reduction in fibrosis.
41. The method of any one of the preceding embodiments, wherein the reduction in fibrosis comprises a reduction in pathology.
42. The method of any one of the preceding embodiments, wherein the reduction in pathology comprises a reduction in renal pathology, and wherein the reduction in renal pathology comprises a reduction in interstitial nephritis, collagen fiber deposition, and nephropathy.
43. The method of any one of the preceding embodiments, wherein the reduction in fibrosis comprises a reduction in inflammatory tissue biomarkers.
44. The method of any one of the preceding embodiments, wherein the inflammatory tissue biomarkers include Col1A1, TGF-b1, MCP-1, IL-1b, IL-6, IL-17, and Timp1.
45. The method of any one of the preceding embodiments, wherein there is a reduction in lung fibrosis in fibrotic subjects.
46. The method of any one of the preceding embodiments, wherein there is a reduction in fibrotic tissue deposition.
47. The method of any one of the preceding embodiments, wherein there is a reduction in hydroxyproline levels in fibrotic subjects.
48. The method of any one of the preceding embodiments, wherein there is an enhancement of blood oxygen saturation and functional lung volume in fibrotic subjects.
49. The method of any one of the preceding embodiments, wherein the compound, tautomer, stereoisomer or mixture of stereoisomers, pharmaceutically acceptable salt, hydrate, deuterated derivative, or N-oxide thereof is administered in an amount sufficient to reduce or reverse fibrotic remodelling in the lungs relative to a bleomycin stimulated vehicle-treated subject.
50. The method of any one of the preceding embodiments, wherein the compound, tautomer, stereoisomer or mixture of stereoisomers, pharmaceutically acceptable salt, hydrate, deuterated derivative, or N-oxide thereof is administered in an amount sufficient to reduce interstitial fibrosis and subacute interstitial inflammation relative to a bleomycin stimulated and vehicle-treated subject.
51. A method for the treatment of a fibrotic disease, the method comprising orally administering to a subject in need thereof a therapeutically effective amount of a compound that is selected from:

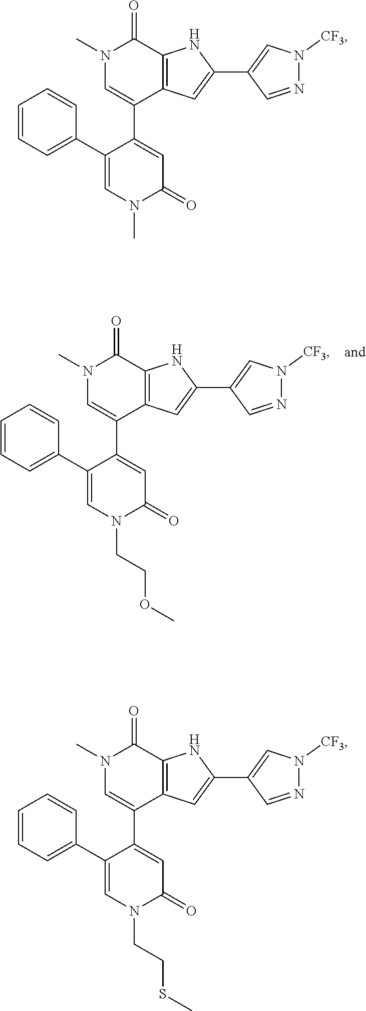 [SMILES: Cn1cc(-c2ccccc2)c(-c2cn(C)c(=O)c3[nH]c(-c4cnn(C(F)(F)F)c4)cc23)cc1=O ; COCCn1cc(-c2ccccc2)c(-c2cn(C)c(=O)c3[nH]c(-c4cnn(C(F)(F)F)c4)cc23)cc1=O ; CSCCn1cc(-c2ccccc2)c(-c2cn(C)c(=O)c3[nH]c(-c4cnn(C(F)(F)F)c4)cc23)cc1=O]

or a tautomer, stereoisomer or mixture of stereoisomers, pharmaceutically acceptable salt, hydrate, deuterated derivative, or N-oxide thereof.
52. A method for the treatment of a fibrotic disease, the method comprising orally administering to a subject in need thereof a therapeutically effective amount of a compound that is

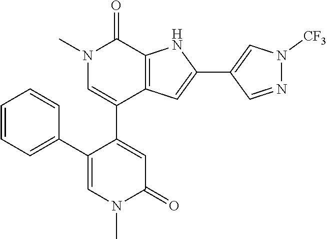 [SMILES: Cn1cc(-c2ccccc2)c(-c2cn(C)c(=O)c3[nH]c(-c4cnn(C(F)(F)F)c4)cc23)cc1=O]

or a tautomer, stereoisomer or mixture of stereoisomers, pharmaceutically acceptable salt, hydrate, deuterated derivative, or N-oxide thereof.
53. A method for the treatment of a fibrotic disease, the method comprising orally administering to a subject in need thereof a therapeutically effective amount of a compound that is

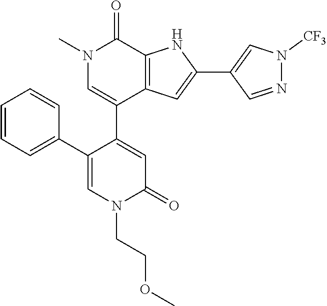 [SMILES: COCCn1cc(-c2ccccc2)c(-c2cn(C)c(=O)c3[nH]c(-c4cnn(C(F)(F)F)c4)cc23)cc1=O]

or a tautomer, stereoisomer or mixture of stereoisomers, pharmaceutically acceptable salt, hydrate, deuterated derivative, or N-oxide thereof.
54. A method for the treatment of a fibrotic disease, the method comprising orally administering to a subject in need thereof a therapeutically effective amount of a compound that is

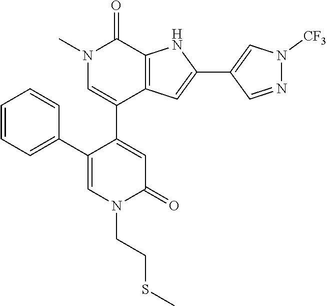 [SMILES: CSCCn1cc(-c2ccccc2)c(-c2cn(C)c(=O)c3[nH]c(-c4cnn(C(F)(F)F)c4)cc23)cc1=O]

or a tautomer, stereoisomer or mixture of stereoisomers, pharmaceutically acceptable salt, hydrate, deuterated derivative, or N-oxide thereof.
55. A method for the treatment of renal fibrosis, the method comprising orally administering to a subject in need thereof a therapeutically effective amount of a compound that is selected from:

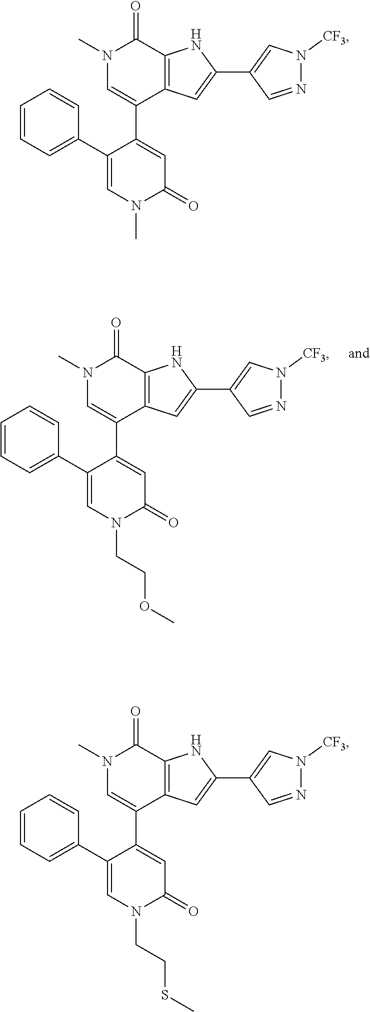 [SMILES: Cn1cc(-c2ccccc2)c(-c2cn(C)c(=O)c3[nH]c(-c4cnn(C(F)(F)F)c4)cc23)cc1=O ; COCCn1cc(-c2ccccc2)c(-c2cn(C)c(=O)c3[nH]c(-c4cnn(C(F)(F)F)c4)cc23)cc1=O ; "and" ; CSCCn1cc(-c2ccccc2)c(-c2cn(C)c(=O)c3[nH]c(-c4cnn(C(F)(F)F)c4)cc23)cc1=O]

or a tautomer, stereoisomer or mixture of stereoisomers, pharmaceutically acceptable salt, hydrate, deuterated derivative, or N-oxide thereof, wherein, upon administration of the therapeutically effective amount of the compound, tautomer, stereoisomer or mixture of stereoisomers, pharmaceutically acceptable salt, hydrate, deuterated derivative, or N-oxide thereof, a therapeutic effect is associated with a reduction in fibrosis.
56. A method for the treatment of renal fibrosis, the method comprising orally administering to a subject in need thereof a therapeutically effective amount of a compound that is

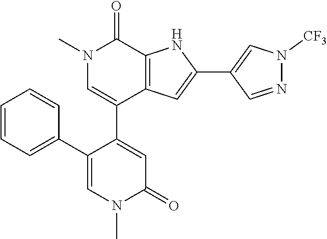 [SMILES: Cn1cc(-c2ccccc2)c(-c2cn(C)c(=O)c3[nH]c(-c4cnn(C(F)(F)F)c4)cc23)cc1=O]

or a tautomer, stereoisomer or mixture of stereoisomers, pharmaceutically acceptable salt, hydrate, deuterated derivative, or N-oxide thereof, wherein, upon administration of a therapeutically effective amount of the compound, tautomer, stereoisomer or mixture of stereoisomers, pharmaceutically acceptable salt, hydrate, deuterated derivative, or N-oxide thereof, a therapeutic effect is associated with a reduction in fibrosis.
57. A method for the treatment of renal fibrosis, the method comprising orally administering to a subject in need thereof a therapeutically effective amount of a compound that is

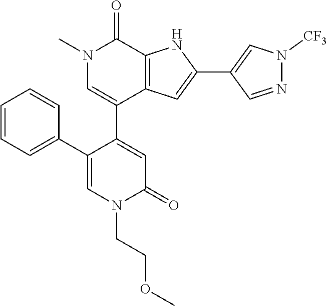 [SMILES: COCCn1cc(-c2ccccc2)c(-c2cn(C)c(=O)c3[nH]c(-c4cnn(C(F)(F)F)c4)cc23)cc1=O]

or a tautomer, stereoisomer or mixture of stereoisomers, pharmaceutically acceptable salt, hydrate, deuterated derivative, or N-oxide thereof, wherein, upon administration of the therapeutically effective amount of the compound, tautomer, stereoisomer or mixture of stereoisomers, pharmaceutically acceptable salt, hydrate, deuterated derivative, or N-oxide thereof, a therapeutic effect is associated with a reduction in fibrosis.
58. A method for the treatment of renal fibrosis, the method comprising orally administering to a subject in need thereof a therapeutically effective amount of a compound that is

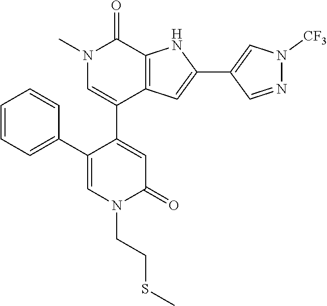 [SMILES: CSCCn1cc(-c2ccccc2)c(-c2cn(C)c(=O)c3[nH]c(-c4cnn(C(F)(F)F)c4)cc23)cc1=O]

or a tautomer, stereoisomer or mixture of stereoisomers, pharmaceutically acceptable salt, hydrate, deuterated derivative, or N-oxide thereof, wherein, upon administration of the therapeutically effective amount of the compound, tautomer, stereoisomer or mixture of stereoisomers, pharmaceutically acceptable salt, hydrate, deuterated derivative, or N-oxide thereof, a therapeutic effect is associated with a reduction in fibrosis.
59. A method for the treatment of pulmonary fibrosis, the method comprising orally administering to a subject in need thereof a therapeutically effective amount of a compound that is selected from:

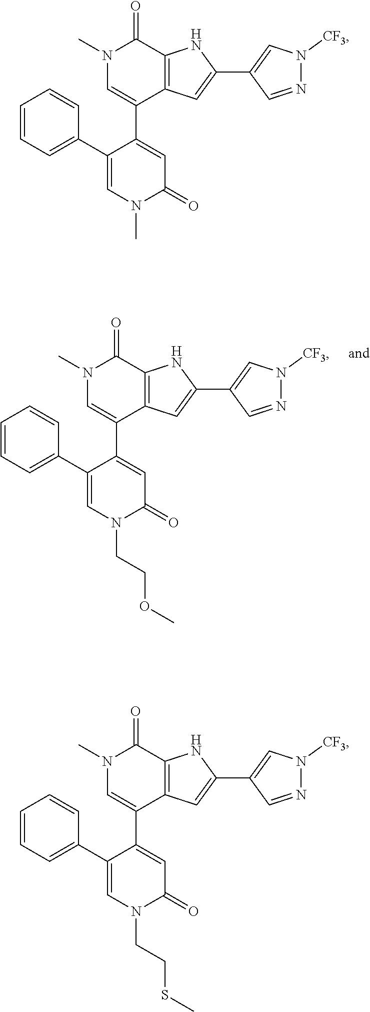 [SMILES: Cn1cc(-c2ccccc2)c(-c2cn(C)c(=O)c3[nH]c(-c4cnn(C(F)(F)F)c4)cc23)cc1=O ; COCCn1cc(-c2ccccc2)c(-c2cn(C)c(=O)c3[nH]c(-c4cnn(C(F)(F)F)c4)cc23)cc1=O ; "and" ; CSCCn1cc(-c2ccccc2)c(-c2cn(C)c(=O)c3[nH]c(-c4cnn(C(F)(F)F)c4)cc23)cc1=O]

or a tautomer, stereoisomer or mixture of stereoisomers, pharmaceutically acceptable salt, hydrate, deuterated derivative, or N-oxide thereof, wherein, upon administration of the therapeutically effective amount of the compound, tautomer, stereoisomer or mixture of stereoisomers, pharmaceutically acceptable salt, hydrate, deuterated derivative, or N-oxide thereof, a therapeutic effect is associated with a reduction in fibrosis.
60. A method for the treatment of pulmonary fibrosis, the method comprising orally administering to a subject in need thereof a therapeutically effective amount of a compound that is

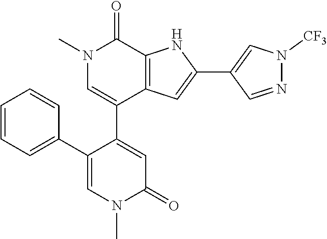 [SMILES: Cn1cc(-c2ccccc2)c(-c2cn(C)c(=O)c3[nH]c(-c4cnn(C(F)(F)F)c4)cc23)cc1=O]

or a tautomer, stereoisomer or mixture of stereoisomers, pharmaceutically acceptable salt, hydrate, deuterated derivative, or N-oxide thereof, wherein, upon administration of a therapeutically effective amount of the compound, tautomer, stereoisomer or mixture of stereoisomers, pharmaceutically acceptable salt, hydrate, deuterated derivative, or N-oxide thereof, a therapeutic effect is associated with a reduction in fibrosis.
61. A method for the treatment of pulmonary fibrosis, the method comprising orally administering to a subject in need thereof a therapeutically effective amount of a compound that is

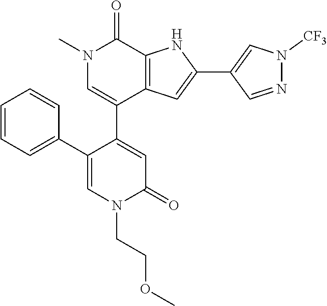 [SMILES: COCCn1cc(-c2ccccc2)c(-c2cn(C)c(=O)c3[nH]c(-c4cnn(C(F)(F)F)c4)cc23)cc1=O]

or a tautomer, stereoisomer or mixture of stereoisomers, pharmaceutically acceptable salt, hydrate, deuterated derivative, or N-oxide thereof, wherein, upon administration of the therapeutically effective amount of the compound, tautomer, stereoisomer or mixture of stereoisomers, pharmaceutically acceptable salt, hydrate, deuterated derivative, or N-oxide thereof, a therapeutic effect is associated with a reduction in fibrosis.
62. A method for the treatment of pulmonary fibrosis, the method comprising orally administering to a subject in need thereof a therapeutically effective amount of a compound that is

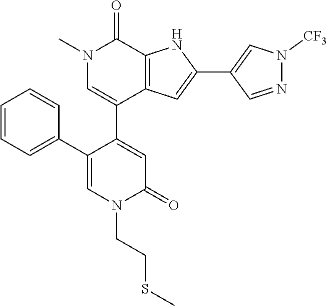 [SMILES: CSCCn1cc(-c2ccccc2)c(-c2cn(C)c(=O)c3[nH]c(-c4cnn(C(F)(F)F)c4)cc23)cc1=O]

or a tautomer, stereoisomer or mixture of stereoisomers, pharmaceutically acceptable salt, hydrate, deuterated derivative, or N-oxide thereof, wherein, upon administration of the therapeutically effective amount of the compound, tautomer, stereoisomer or mixture of stereoisomers, pharmaceutically acceptable salt, hydrate, deuterated derivative, or N-oxide thereof, a therapeutic effect is associated with a reduction in fibrosis.
63. The method of any one of the preceding embodiments, wherein the progression of fibrosis severity is slowed or retarded.
64. The method of any one of the preceding embodiments, wherein the appearance or increase of one or more indicators of fibrosis severity is slowed or retarded.
65. The method of any one of the preceding embodiments, wherein the disease or disorder is a lupus disease or disorder.
66. The method of any one of the preceding embodiments, wherein the disease or disorder is systemic lupus erythematosus (SLE).
67. The method of any one of the preceding embodiments, wherein the disease or disorder is cutaneous lupus erythematosus (CLE).
68. The method of any one of the preceding embodiments, wherein, upon administration of the therapeutically effective amount of the compound, tautomer, stereoisomer or mixture of stereoisomers, pharmaceutically acceptable salt, hydrate, deuterated derivative, or N-oxide thereof, there is a therapeutic effect associated with a reduction in proteinuria.
69. The method of any one of the preceding embodiments, wherein, upon administration of the therapeutically effective amount of the compound, tautomer, stereoisomer or mixture of stereoisomers, pharmaceutically acceptable salt, hydrate, deuterated derivative, or N-oxide thereof, there is a therapeutic effect associated with a reduction in pathology.
70. The method of any one of the preceding embodiments, wherein, upon administration of the therapeutically effective amount of the compound, tautomer, stereoisomer or mixture of stereoisomers, pharmaceutically acceptable salt, hydrate, deuterated derivative, or N-oxide thereof, there is a therapeutic effect associated with a reduction in glomerular lesions, tubular and interstitial lesions, and total kidney lesions.
71. The method of any one of the preceding embodiments, wherein, upon administration of the therapeutically effective amount of the compound, tautomer, stereoisomer or mixture of stereoisomers, pharmaceutically acceptable salt, hydrate, deuterated derivative, or N-oxide thereof, there is a therapeutic effect associated with a reduction in blood urea nitrogen (BUN).
72. The method of any one of the preceding embodiments, wherein, upon administration of the therapeutically effective amount of the compound, tautomer, stereoisomer or mixture of stereoisomers, pharmaceutically acceptable salt, hydrate, deuterated derivative, or N-oxide thereof, there is a therapeutic effect associated with a reduction in anti-dsDNA antibodies.
73. The method of any one of the preceding embodiments, wherein there is a reduction in one or more inflammatory tissue biomarkers.
74. The method of any one of the preceding embodiments, wherein there is a reduction in severity of the disease or disorder.
75. The method of any one of the preceding embodiments, wherein the disease or disorder is a skin disorder.
76. The method of any one of the preceding embodiments, wherein the disease or disorder is psoriasis.
77. The method of any one of the preceding embodiments, wherein, upon administration of the therapeutically effective amount of the compound, tautomer, stereoisomer or mixture of stereoisomers, pharmaceutically acceptable salt, hydrate, deuterated derivative, or N-oxide thereof, there is a therapeutic effect associated with a reduction in inflammation.
78. The method of any one of the preceding embodiments, wherein the reduction in inflammation comprises a reduction in pathology.
79. The method of any one of the preceding embodiments, wherein the reduction in pathology comprises a reduction in a psoriasis area and severity index score.
80. The method of any one of the preceding embodiments, wherein the reduction follows and/or is accompanied by a reduction in inflammatory tissue biomarkers.
81. The method of any one of the preceding embodiments, wherein the inflammatory tissue biomarkers are associated with psoriasis.
82. The method of any one of the preceding embodiments, wherein the inflammatory tissue biomarkers include one or more of IL-17, IL-22, IL-1β, IL-6, TNF-α, and IL-23.
83. A method for the treatment of a skin disease, the method comprising orally administering to a subject in need thereof a therapeutically effective amount of a compound that is selected from:

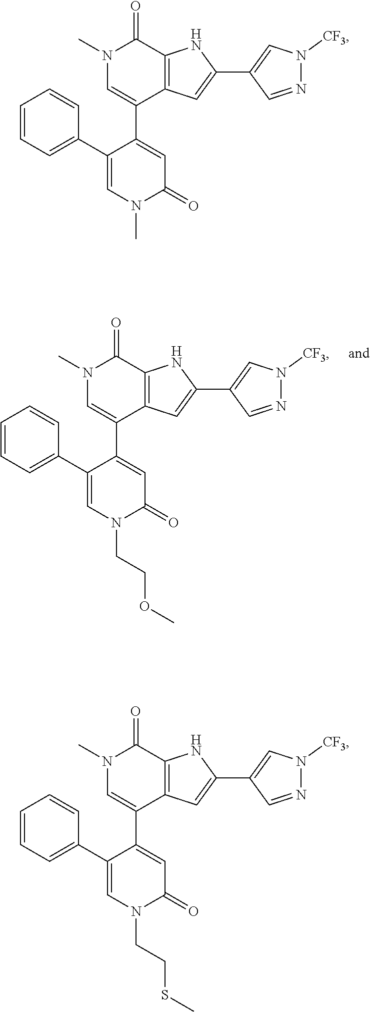 [SMILES: Cn1cc(-c2ccccc2)c(-c2cn(C)c(=O)c3[nH]c(-c4cnn(C(F)(F)F)c4)cc23)cc1=O ; COCCn1cc(-c2ccccc2)c(-c2cn(C)c(=O)c3[nH]c(-c4cnn(C(F)(F)F)c4)cc23)cc1=O ; "and" ; CSCCn1cc(-c2ccccc2)c(-c2cn(C)c(=O)c3[nH]c(-c4cnn(C(F)(F)F)c4)cc23)cc1=O]

or a tautomer, stereoisomer or mixture of stereoisomers, pharmaceutically acceptable salt, hydrate, deuterated derivative, or N-oxide thereof.
84. A method for the treatment of a skin disease, the method comprising orally administering to a subject in need thereof a therapeutically effective amount of a compound that is

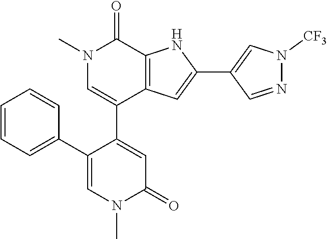 [SMILES: Cn1cc(-c2ccccc2)c(-c2cn(C)c(=O)c3[nH]c(-c4cnn(C(F)(F)F)c4)cc23)cc1=O]

or a tautomer, stereoisomer or mixture of stereoisomers, pharmaceutically acceptable salt, hydrate, deuterated derivative, or N-oxide thereof.
85. A method for the treatment of a skin disease, the method comprising orally administering to a subject in need thereof a therapeutically effective amount of a compound that is

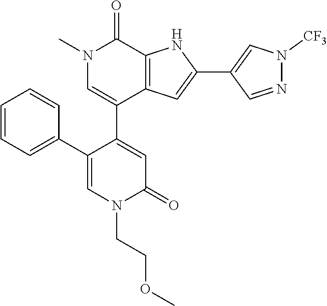 [SMILES: COCCn1cc(-c2ccccc2)c(-c2cn(C)c(=O)c3[nH]c(-c4cnn(C(F)(F)F)c4)cc23)cc1=O]

or a tautomer, stereoisomer or mixture of stereoisomers, pharmaceutically acceptable salt, hydrate, deuterated derivative, or N-oxide thereof.
86. A method for the treatment of a skin disease, the method comprising orally administering to a subject in need thereof a therapeutically effective amount of a compound that is

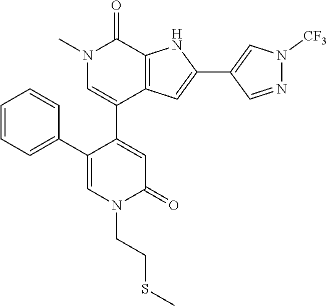 [SMILES: CSCCn1cc(-c2ccccc2)c(-c2cn(C)c(=O)c3[nH]c(-c4cnn(C(F)(F)F)c4)cc23)cc1=O]

or a tautomer, stereoisomer or mixture of stereoisomers, pharmaceutically acceptable salt, hydrate, deuterated derivative, or N-oxide thereof.
87. A method for the treatment of psoriasis, the method comprising orally administering to a subject in need thereof a therapeutically effective amount of a compound that is selected from:

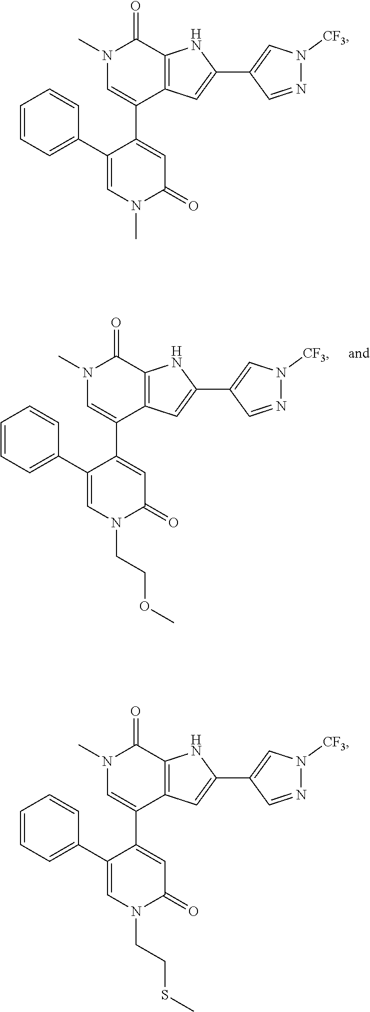 [SMILES: Cn1cc(-c2ccccc2)c(-c2cn(C)c(=O)c3[nH]c(-c4cnn(C(F)(F)F)c4)cc23)cc1=O ; COCCn1cc(-c2ccccc2)c(-c2cn(C)c(=O)c3[nH]c(-c4cnn(C(F)(F)F)c4)cc23)cc1=O ; "and" ; CSCCn1cc(-c2ccccc2)c(-c2cn(C)c(=O)c3[nH]c(-c4cnn(C(F)(F)F)c4)cc23)cc1=O]

or a tautomer, stereoisomer or mixture of stereoisomers, pharmaceutically acceptable salt, hydrate, deuterated derivative, or N-oxide thereof, wherein, upon administration of the therapeutically effective amount of the compound, tautomer, stereoisomer or mixture of stereoisomers, pharmaceutically acceptable salt, hydrate, deuterated derivative, or N-oxide thereof, a therapeutic effect is associated with a reduction in psoriasis.
88. A method for the treatment of psoriasis, the method comprising orally administering to a subject in need thereof a therapeutically effective amount of a compound that is

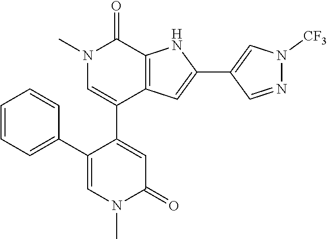 [SMILES: Cn1cc(-c2ccccc2)c(-c2cn(C)c(=O)c3[nH]c(-c4cnn(C(F)(F)F)c4)cc23)cc1=O]

or a tautomer, stereoisomer or mixture of stereoisomers, pharmaceutically acceptable salt, hydrate, deuterated derivative, or N-oxide thereof, wherein, upon administration of the therapeutically effective amount of the compound, tautomer, stereoisomer or mixture of stereoisomers, pharmaceutically acceptable salt, hydrate, deuterated derivative, or N-oxide thereof, a therapeutic effect is associated with a reduction in psoriasis.
89. A method for the treatment of psoriasis, the method comprising orally administering to a subject in need thereof a therapeutically effective amount of a compound that is

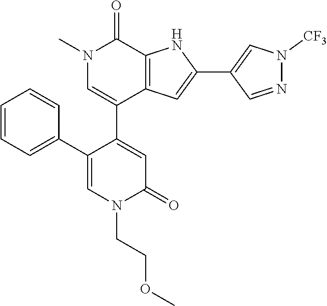 [SMILES: COCCn1cc(-c2ccccc2)c(-c2cn(C)c(=O)c3[nH]c(-c4cnn(C(F)(F)F)c4)cc23)cc1=O]

or a tautomer, stereoisomer or mixture of stereoisomers, pharmaceutically acceptable salt, hydrate, deuterated derivative, or N-oxide thereof, wherein, upon administration of the therapeutically effective amount of the compound, tautomer, stereoisomer or mixture of stereoisomers, pharmaceutically acceptable salt, hydrate, deuterated derivative, or N-oxide thereof, a therapeutic effect is associated with a reduction in psoriasis.
90. A method for the treatment of psoriasis, the method comprising orally administering to a subject in need thereof a therapeutically effective amount of a compound that is

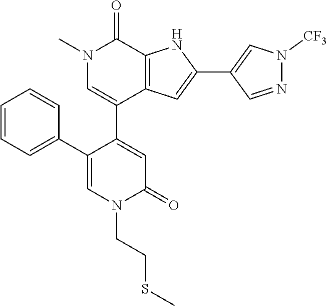 [SMILES: CSCCn1cc(-c2ccccc2)c(-c2cn(C)c(=O)c3[nH]c(-c4cnn(C(F)(F)F)c4)cc23)cc1=O]

or a tautomer, stereoisomer or mixture of stereoisomers, pharmaceutically acceptable salt, hydrate, deuterated derivative, or N-oxide thereof, wherein, upon administration of the therapeutically effective amount of the compound, tautomer, stereoisomer or mixture of stereoisomers, pharmaceutically acceptable salt, hydrate, deuterated derivative, or N-oxide thereof, a therapeutic effect is associated with a reduction in psoriasis.
91. The method of any one of the preceding embodiments, wherein the compound is

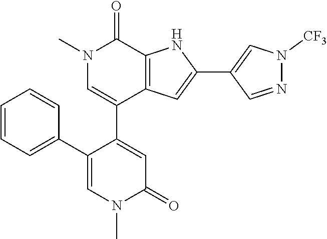 [SMILES: Cn1cc(-c2ccccc2)c(-c2cn(C)c(=O)c3[nH]c(-c4cnn(C(F)(F)F)c4)cc23)cc1=O]

or a pharmaceutically acceptable salt thereof.
92. The method of any one of the preceding embodiments, wherein the compound is

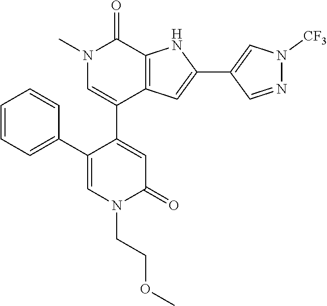 [SMILES: COCCn1cc(-c2ccccc2)c(-c2cn(C)c(=O)c3[nH]c(-c4cnn(C(F)(F)F)c4)cc23)cc1=O]

or a pharmaceutically acceptable salt thereof.
93. The method of any one of the preceding embodiments, wherein the compound is

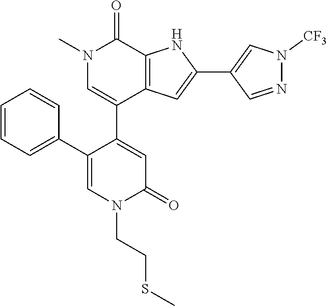 [SMILES: CSCCn1cc(-c2ccccc2)c(-c2cn(C)c(=O)c3[nH]c(-c4cnn(C(F)(F)F)c4)cc23)cc1=O]

or a pharmaceutically acceptable salt thereof.
94. The method of any one of the preceding embodiments, wherein the progression of psoriasis is slowed or retarded.
95. The method of any one of the preceding embodiments, wherein the appearance or increase of one or more indicators of psoriasis severity is slowed or retarded.
96. A method for the treatment of an inflammatory disease, the method comprising orally administering to a subject in need thereof a therapeutically effective amount of a compound that is selected from:

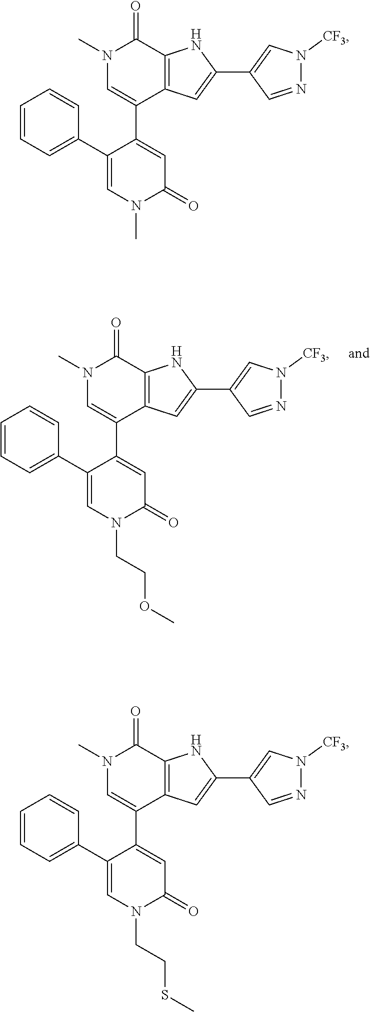 [SMILES: Cn1cc(-c2ccccc2)c(-c2cn(C)c(=O)c3[nH]c(-c4cnn(C(F)(F)F)c4)cc23)cc1=O ; COCCn1cc(-c2ccccc2)c(-c2cn(C)c(=O)c3[nH]c(-c4cnn(C(F)(F)F)c4)cc23)cc1=O ; "and" ; CSCCn1cc(-c2ccccc2)c(-c2cn(C)c(=O)c3[nH]c(-c4cnn(C(F)(F)F)c4)cc23)cc1=O]

or a tautomer, stereoisomer or mixture of stereoisomers, pharmaceutically acceptable salt, hydrate, deuterated derivative, or N-oxide thereof, wherein, upon administration of the therapeutically effective amount of the compound, tautomer, stereoisomer or mixture of stereoisomers, pharmaceutically acceptable salt, hydrate, deuterated derivative, or N-oxide thereof, a therapeutic effect is associated with a reduction of IL-17A and/or IL-22, and wherein the IC50 thereof is more than about 7,000-fold or more than about 10,000-fold less or between about 7,000 less to about 17,000 less than the IC50 for CXCL10.
97. A method for the treatment of a skin disease, the method comprising orally administering to a subject in need thereof a therapeutically effective amount of a compound that is

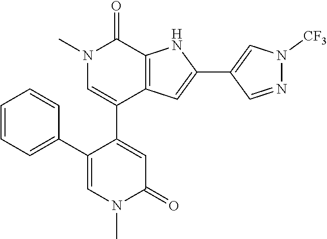 [SMILES: Cn1cc(-c2ccccc2)c(-c2cn(C)c(=O)c3[nH]c(-c4cnn(C(F)(F)F)c4)cc23)cc1=O]

or a tautomer, stereoisomer or mixture of stereoisomers, pharmaceutically acceptable salt, hydrate, deuterated derivative, or N-oxide thereof, wherein, upon administration of the therapeutically effective amount of the compound, tautomer, stereoisomer or mixture of stereoisomers, pharmaceutically acceptable salt, hydrate, deuterated derivative, or N-oxide thereof, a therapeutic effect is associated with a reduction of IL-17A and/or IL-22, and wherein the IC50 thereof is more than about 7,000-fold or more than about 10,000-fold less or between about 7,000 less to about 17,000 less than the IC50 for CXCL10.
98. A method for the treatment of a skin disease, the method comprising orally administering to a subject in need thereof a therapeutically effective amount of a compound that is

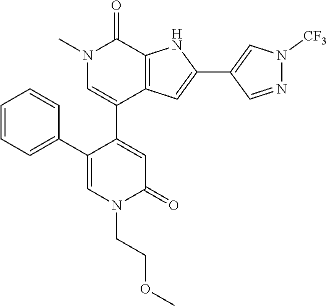 [SMILES: COCCn1cc(-c2ccccc2)c(-c2cn(C)c(=O)c3[nH]c(-c4cnn(C(F)(F)F)c4)cc23)cc1=O]

or a tautomer, stereoisomer or mixture of stereoisomers, pharmaceutically acceptable salt, hydrate, deuterated derivative, or N-oxide thereof, wherein, upon administration of the therapeutically effective amount of the compound, tautomer, stereoisomer or mixture of stereoisomers, pharmaceutically acceptable salt, hydrate, deuterated derivative, or N-oxide thereof, a therapeutic effect is associated with a reduction of IL-17A and/or IL-22, and wherein the IC50 thereof is more than about 7,000-fold or more than about 10,000-fold less or between about 7,000 less to about 17,000 less than the IC50 for CXCL10.
99. A method for the treatment of a skin disease, the method comprising orally administering to a subject in need thereof a therapeutically effective amount of a compound that is

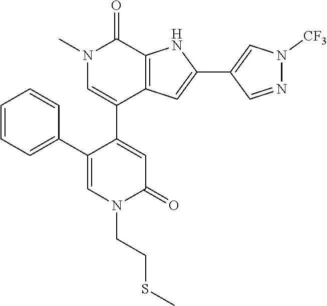 [SMILES: CSCCn1cc(-c2ccccc2)c(-c2cn(C)c(=O)c3[nH]c(-c4cnn(C(F)(F)F)c4)cc23)cc1=O]

or a tautomer, stereoisomer or mixture of stereoisomers, pharmaceutically acceptable salt, hydrate, deuterated derivative, or N-oxide thereof, wherein, upon administration of the therapeutically effective amount of the compound, tautomer, stereoisomer or mixture of stereoisomers, pharmaceutically acceptable salt, hydrate, deuterated derivative, or N-oxide thereof, a therapeutic effect is associated with a reduction of IL-17A and/or IL-22, and wherein the IC50 thereof is more than about 7,000-fold or more than about 10,000-fold less or between about 7,000 less to about 17,000 less than the IC50 for CXCL10.
100. The method of any one of the preceding embodiments, wherein the disease or disorder is a CNS disease or disorder.
101. The method of any one of the preceding embodiments, wherein the CNS disease or disorder is an autoimmune disease that attacks the central nervous system (CNS), a CNS inflammation, and/or a demyelinating disease.
102. The method of any one of the preceding embodiments, wherein the demyelinating disease is multiple sclerosis (MS) and/or a MS-related condition.
103. The method of any one of the preceding embodiments, wherein the severity of the CNS disease or disorder is reduced.
104. The method of any one of the preceding embodiments, wherein levels of one or more inflammatory biomarkers are reduced.
105. The method of any one of the preceding embodiments, wherein the one or more inflammatory biomarkers are associated with a CNS disease or disorder.
106. The method of any one of the preceding embodiments, wherein the one or more inflammatory biomarkers are associated with multiple sclerosis (MS) and/or a MS-related condition.
107. The method of any one of the preceding embodiments, wherein the one or more inflammatory biomarkers include one or more of IFNγ and IL-12/IL-23p40.
108. The method of any one of the preceding embodiments, wherein CNS inflammation is suppressed.
109. The method of any one of the preceding embodiments, wherein the progression of CNS inflammation is limited, slowed, and/or retarded.
110. The method of any one of the preceding embodiments, wherein the progression of demyelination is limited, slowed, and/or retarded.
111. The method of any one of the preceding embodiments, wherein the progression of autoimmune diseases that attack the CNS is limited, slowed, and/or retarded.
112. The method of any one of the preceding embodiments, wherein the disease course is reversed (e.g., in part) facilitating a recovery.
113. The method of any one of the preceding embodiments, wherein immune cell infiltration into the CNS is modulated.
114. The method of any one of the preceding embodiments, wherein the severity of axonal damage is reduced.
115. The method of any one of the preceding embodiments, wherein the progression of axonal damage is limited, slowed and/or retarded.
116. A method for treating multiple sclerosis, the method comprising orally administering to a subject in need thereof a therapeutically effective amount of a compound selected from:

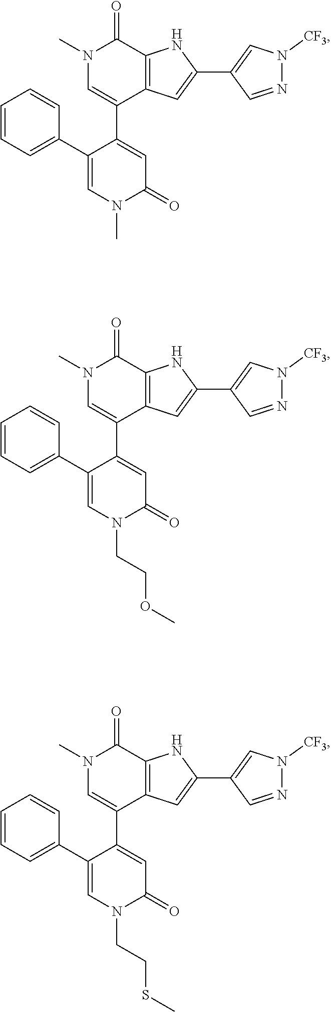 [SMILES: Cn1cc(-c2ccccc2)c(-c2cn(C)c(=O)c3[nH]c(-c4cnn(C(F)(F)F)c4)cc23)cc1=O ; COCCn1cc(-c2ccccc2)c(-c2cn(C)c(=O)c3[nH]c(-c4cnn(C(F)(F)F)c4)cc23)cc1=O ; CSCCn1cc(-c2ccccc2)c(-c2cn(C)c(=O)c3[nH]c(-c4cnn(C(F)(F)F)c4)cc23)cc1=O]

or tautomers, stereoisomers or mixture of stereoisomers, pharmaceutically acceptable salts, hydrates, deuterated derivatives, and N-oxides thereof.
117. A method for the treatment of multiple sclerosis, the method comprising orally administering to a subject in need thereof a therapeutically effective amount of a compound selected from

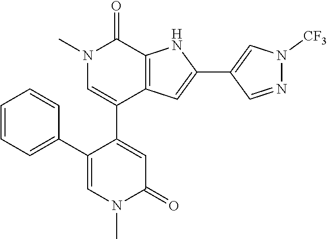 [SMILES: Cn1cc(-c2ccccc2)c(-c2cn(C)c(=O)c3[nH]c(-c4cnn(C(F)(F)F)c4)cc23)cc1=O]

or tautomers, stereoisomers or mixture of stereoisomers, pharmaceutically acceptable salts, hydrates, deuterated derivatives, or N-oxides thereof.
118. A method for the treatment of multiple sclerosis, the method comprising orally administering to a subject in need thereof a therapeutically effective amount of a compound selected from:

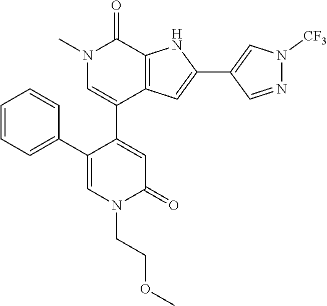 [SMILES: COCCn1cc(-c2ccccc2)c(-c2cn(C)c(=O)c3[nH]c(-c4cnn(C(F)(F)F)c4)cc23)cc1=O]

or tautomers, stereoisomers or mixture of stereoisomers, pharmaceutically acceptable salts, hydrates, deuterated derivatives, or N-oxides thereof.
119. A method for the treatment of multiple sclerosis, the method comprising orally administering to a subject in need thereof a therapeutically effective amount of a compound selected from:

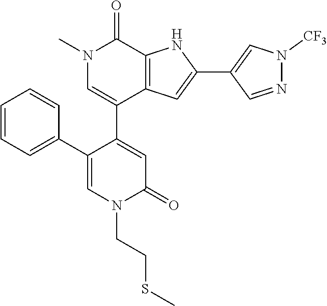 [SMILES: CSCCn1cc(-c2ccccc2)c(-c2cn(C)c(=O)c3[nH]c(-c4cnn(C(F)(F)F)c4)cc23)cc1=O]

or tautomers, stereoisomers or mixture of stereoisomers, pharmaceutically acceptable salts, hydrates, deuterated derivatives, or N-oxides thereof.
120. The method of any one of the preceding embodiments, wherein progression of multiple sclerosis is slowed or retarded.
121. The method of any one of the preceding embodiments, wherein the compound or a tautomer, stereoisomer or mixture of stereoisomers, pharmaceutically acceptable salt, hydrate, deuterated derivative or an N-oxide thereof is in the form of a pharmaceutical composition which comprises the compound of formula (XXIA), tautomer, stereoisomer or mixture of stereoisomers, pharmaceutically acceptable salt, hydrate, deuterated derivative, or N-oxide thereof, and a pharmaceutically acceptable carrier.
122. The method of any one of the preceding embodiments, wherein the compound, tautomer, stereoisomer or mixture of stereoisomers, pharmaceutically acceptable salt, hydrate, deuterated derivative, or N-oxide thereof is formulated as a suspension or partial suspension in the composition.
123. The method of any one of the preceding embodiments, wherein the compound, tautomer, stereoisomer or mixture of stereoisomers, pharmaceutically acceptable salt, hydrate, deuterated derivative, or N-oxide thereof is micronized.
124. The method of any one of the preceding embodiments, wherein the compound, tautomer, stereoisomer or mixture of stereoisomers, pharmaceutically acceptable salt, hydrate, deuterated derivative, or N-oxide thereof comprises nanoparticles.
125. The method of any one of the preceding embodiments, wherein the compound, tautomer, stereoisomer or mixture of stereoisomers, pharmaceutically acceptable salt, hydrate, deuterated derivative, or N-oxide thereof is solubilized or partially solubilized in the composition.
126. The method of any one of the preceding embodiments, wherein the compound, tautomer, stereoisomer or mixture of stereoisomers, pharmaceutically acceptable salt, hydrate, deuterated derivative, or N-oxide thereof or pharmaceutical composition is administered locally, topically, or systemically.
127. The method of any one of the preceding embodiments, wherein the compound, tautomer, stereoisomer or mixture of stereoisomers, pharmaceutically acceptable salt, hydrate, deuterated derivative, or N-oxide thereof or pharmaceutical composition is administered by injection or infusion.
128. The method of any one of the preceding embodiments, wherein the compound, tautomer, stereoisomer or mixture of stereoisomers, pharmaceutically acceptable salt, hydrate, deuterated derivative, or N-oxide thereof or pharmaceutical composition is administered orally.
129. The method of any one of the preceding embodiments, wherein the compound, tautomer, stereoisomer or mixture of stereoisomers, pharmaceutically acceptable salt, hydrate, deuterated derivative, or N-oxide thereof or pharmaceutical composition has activity against one or more BET domains.

This disclosure additionally includes the following numbered embodiments:
1. A method for the treatment of an inflammatory and/or an autoimmune disease or disorder or a disease or disorder related thereto, the method comprising administering to a subject in need thereof a therapeutically effective amount of a compound of formula (XXI), a tautomer, a stereoisomer or a mixture of stereoisomers, a pharmaceutically acceptable salt, a hydrate, a deuterated derivative, or an N-oxide thereof:

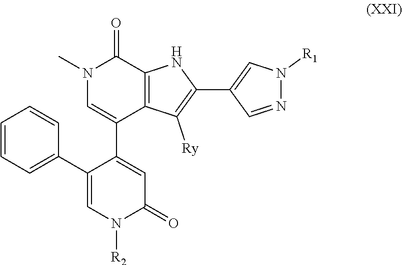

wherein:
R₁ is C₁-C₄-haloalkyl;
R₂ is C₁-C₄-alkyl, wherein the C₁-C₄-alkyl is optionally substituted with SR₃ or OR₃;
R₃ is selected from H, C₁-C₃-alkyl, C(O)—C₁-C₃-alkyl, and C₁-C₃-haloalkyl; and
R_{y} is hydrogen or halo.
2. The method of any one of the preceding embodiments, wherein R₁ is independently selected from CF₃, CHF₂, CH₂CF₃, and CH₂CH₂F.
3. The method of any one of the preceding embodiments, wherein R₁ is independently selected from CBr₃, CHBr₂, CH₂CBr₃, and CH₂CH₂Br.
4. The method of any one of the preceding embodiments, wherein R₁ is independently selected from CCl₃, CHCl₂, CH₂CCl₃, and CH₂CH₂Cl.
5. The method of any one of the preceding embodiments, wherein R₁ is CH₂CH₂F.
6. The method of any one of the preceding embodiments, wherein R¹ is CHF₂.
7. The method of any one of the preceding embodiments, wherein R₁ is CF₃.
8. The method of any one of the preceding embodiments, wherein R₂ is —CH₃.
9. The method of any one of the preceding embodiments, wherein R_{y} is halo.
10. The method of any one of the preceding embodiments, wherein R_{y} is fluorine.
11. The method of any one of the preceding embodiments, wherein the compound of formula (XXI) is:

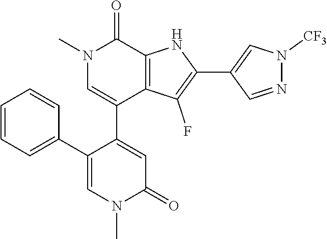 [SMILES: Cn1cc(-c2ccccc2)c(-c2cn(C)c(=O)c3[nH]c(-c4cnn(C(F)(F)F)c4)c(F)c23)cc1=O]

or a tautomer, a stereoisomer or mixture of stereoisomers, a pharmaceutically acceptable salt, a hydrate, a deuterated derivative, or N-oxide thereof.
12. The method of any one of the preceding embodiments, wherein the disease or disorder is a joint or joint-related disorder.
13. The method of any one of the preceding embodiments, wherein the joint related disease or disorder is chosen from arthritis, bursitis, Ehlers-Danlos syndrome, epicondylitis, Felty Syndrome, gouty arthritis, psoriatic arthritis, osteoarthritis, rheumatoid arthritis, Still's disease, tenosynovitis, synovitis, Sjögren's Syndrome, Lyme disease, Whipple disease, bone cancer, lupus, and other autoimmune joint disorders.
14. The method of any one of the preceding embodiments, wherein the joint or joint related disease or disorder comprises an arthritis.
15. The method of any one of the preceding embodiments, wherein the arthritis comprises rheumatoid arthritis.
16. The method of any one of the preceding embodiments, wherein the disorder is an arthritis and upon administration of the therapeutically effective amount of the compound, tautomer, stereoisomer or mixture of stereoisomers, pharmaceutically acceptable salt, hydrate, deuterated derivative, or N-oxide thereof there is a therapeutic effect associated with reduction in inflammation.
17. The method of any one of the preceding embodiments, wherein the therapeutic effect associated with a reduction in inflammation is a reduction in thickness or girth of a joint or limb.
18. The method of any one of the preceding embodiments, wherein there is reduction in arthritic scoring or severity, and wherein the reduction in arthritic scoring or severity is a reduction in:
   (a) definite redness and swelling of an ankle/wrist or apparent redness and swelling limited to individual digits, regardless of the number of affected digits;
   (b) severe redness and swelling of an ankle/wrist;
   (c) redness and swelling of an entire appendage including digits; and/or
   (d) maximally inflamed limb with involvement of multiple joints.
19. The method of any one of the preceding embodiments, wherein the reduction is dose dependent.
20. The method of any one of the preceding embodiments, wherein the reduction is by >about 50%.
21. A method for the treatment of a joint or joint-related disease, the method comprising orally administering to a subject in need thereof a therapeutically effective amount of a compound that is:

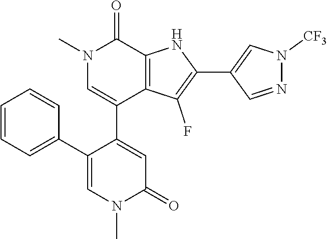 [SMILES: Cn1cc(-c2ccccc2)c(-c2cn(C)c(=O)c3[nH]c(-c4cnn(C(F)(F)F)c4)c(F)c23)cc1=O]

or a tautomer, stereoisomer or mixture of stereoisomers, pharmaceutically acceptable salt, hydrate, deuterated derivative, or N-oxide thereof.
22. A method for the treatment of an arthritic disease, the method comprising orally administering to a subject in need thereof a therapeutically effective amount of a compound that is:

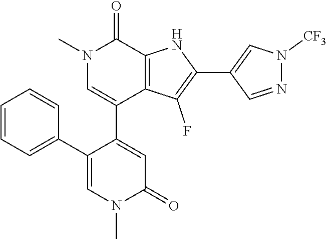 [SMILES: Cn1cc(-c2ccccc2)c(-c2cn(C)c(=O)c3[nH]c(-c4cnn(C(F)(F)F)c4)c(F)c23)cc1=O]

or a tautomer, stereoisomer or mixture of stereoisomers, pharmaceutically acceptable salt, hydrate, deuterated derivative, or N-oxide thereof, wherein upon administration of a therapeutically effective amount of the compound, tautomer, stereoisomer or mixture of stereoisomers, pharmaceutically acceptable salt, hydrate, deuterated derivative, or N-oxide thereof a therapeutic effect is associated with a reduction in inflammation.
23. The method of any one of the preceding embodiments, wherein the disease or disorder is a fibrotic disease or disorder.
24. The method of any one of the preceding embodiments, wherein the disease or disorder is renal fibrosis.
25. The method of any one of the preceding embodiments, wherein the disease or disorder is pulmonary fibrosis (PF).
26. The method of any one of the preceding embodiments, wherein the disease or disorder is idiopathic pulmonary fibrosis (IPF).
27. The method of any one of the preceding embodiments, wherein, upon administration of the therapeutically effective amount of the compound, tautomer, stereoisomer or mixture of stereoisomers, pharmaceutically acceptable salt, hydrate, deuterated derivative, or N-oxide thereof, there is a therapeutic effect associated with a reduction in fibrosis.
28. The method of any one of the preceding embodiments, wherein the reduction in fibrosis comprises a reduction in pathology.
29. The method of any one of the preceding embodiments, wherein the reduction in pathology comprises a reduction in renal pathology, and wherein the reduction in renal pathology comprises a reduction in interstitial nephritis, collagen fiber deposition, and nephropathy.
30. The method of any one of the preceding embodiments, wherein the reduction in fibrosis comprises a reduction in inflammatory tissue biomarkers.
31. The method of any one of the preceding embodiments, wherein the inflammatory tissue biomarkers include Col1A1, TGF-b1, MCP-1, IL-1b, IL-6, IL-17, and Timp1.
32. The method of any one of the preceding embodiments, wherein there is a reduction in lung fibrosis in fibrotic subjects.
33. The method of any one of the preceding embodiments, wherein there is a reduction in fibrotic tissue deposition.
34. The method of any one of the preceding embodiments, wherein there is a reduction in hydroxyproline levels in fibrotic subjects.
35. The method of any one of the preceding embodiments, wherein there is an enhancement of blood oxygen saturation and functional lung volume in fibrotic subjects.
36. The method of any one of the preceding embodiments, wherein the compound, tautomer, stereoisomer or mixture of stereoisomers, pharmaceutically acceptable salt, hydrate, deuterated derivative, or N-oxide thereof is administered in an amount sufficient to reduce or reverse fibrotic remodelling in the lungs relative to a bleomycin stimulated vehicle-treated subject.
37. The method of any one of the preceding embodiments, wherein the compound, tautomer, stereoisomer or mixture of stereoisomers, pharmaceutically acceptable salt, hydrate, deuterated derivative, or N-oxide thereof is administered in an amount sufficient to reduce interstitial fibrosis and subacute interstitial inflammation relative to a bleomycin stimulated vehicle-treated subject.
38. A method for the treatment of a fibrotic disease, the method comprising orally administering to a subject in need thereof a therapeutically effective amount of a compound that is:

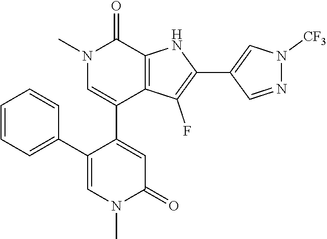 [SMILES: Cn1cc(-c2ccccc2)c(-c2cn(C)c(=O)c3[nH]c(-c4cnn(C(F)(F)F)c4)c(F)c23)cc1=O]

or a tautomer, stereoisomer or mixture of stereoisomers, pharmaceutically acceptable salt, hydrate, deuterated derivative, or N-oxide thereof.
39. A method for the treatment of renal fibrosis, the method comprising orally administering to a subject in need thereof a therapeutically effective amount of a compound that is:

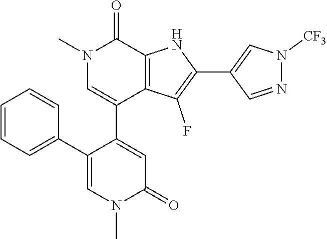 [SMILES: Cn1cc(-c2ccccc2)c(-c2cn(C)c(=O)c3[nH]c(-c4cnn(C(F)(F)F)c4)c(F)c23)cc1=O]

or a tautomer, stereoisomer or mixture of stereoisomers, pharmaceutically acceptable salt, hydrate, deuterated derivative, or N-oxide thereof, wherein, upon administration of the therapeutically effective amount of the compound, tautomer, stereoisomer or mixture of stereoisomers, pharmaceutically acceptable salt, hydrate, deuterated derivative, or N-oxide thereof, a therapeutic effect is associated with a reduction in fibrosis.
40. A method for the treatment of pulmonary fibrosis, the method comprising orally administering to a subject in need thereof a therapeutically effective amount of a compound that is:

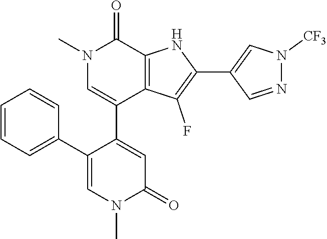 [SMILES: Cn1cc(-c2ccccc2)c(-c2cn(C)c(=O)c3[nH]c(-c4cnn(C(F)(F)F)c4)c(F)c23)cc1=O]

or a tautomer, stereoisomer or mixture of stereoisomers, pharmaceutically acceptable salt, hydrate, deuterated derivative, or N-oxide thereof, wherein, upon administration of the therapeutically effective amount of the compound, tautomer, stereoisomer or mixture of stereoisomers, pharmaceutically acceptable salt, hydrate, deuterated derivative, or N-oxide thereof, a therapeutic effect is associated with a reduction in fibrosis.
41. The method of any one of the preceding embodiments, wherein the progression of fibrosis severity is slowed or retarded.
42. The method of any one of the preceding embodiments, wherein the appearance or increase of one or more indicators of fibrosis severity is slowed or retarded.
43. The method of any one of the preceding embodiments, wherein the disease or disorder is a lupus disease or disorder.
44. The method of any one of the preceding embodiments, wherein the disease or disorder is systemic lupus erythematosus (SLE).
45. The method of any one of the preceding embodiments, wherein the disease or disorder is cutaneous lupus erythematosus (CLE).
46. The method of any one of the preceding embodiments, wherein, upon administration of the therapeutically effective amount of the compound, tautomer, stereoisomer or mixture of stereoisomers, pharmaceutically acceptable salt, hydrate, deuterated derivative, or N-oxide thereof, there is a therapeutic effect associated with a reduction in proteinuria.
47. The method of any one of the preceding embodiments, wherein, upon administration of the therapeutically effective amount of the compound, tautomer, stereoisomer or mixture of stereoisomers, pharmaceutically acceptable salt, hydrate, deuterated derivative, or N-oxide thereof, there is a therapeutic effect associated with a reduction in pathology.
48. The method of any one of the preceding embodiments, wherein, upon administration of the therapeutically effective amount of the compound, tautomer, stereoisomer or mixture of stereoisomers, pharmaceutically acceptable salt, hydrate, deuterated derivative, or N-oxide thereof, there is a therapeutic effect associated with a reduction in glomerular lesions, tubular and interstitial lesions, and total kidney lesions.
49. The method of any one of the preceding embodiments, wherein, upon administration of the therapeutically effective amount of the compound, tautomer, stereoisomer or mixture of stereoisomers, pharmaceutically acceptable salt, hydrate, deuterated derivative, or N-oxide thereof, there is a therapeutic effect associated with a reduction in blood urea nitrogen (BUN).
50. The method of any one of the preceding embodiments, wherein, upon administration of the therapeutically effective amount of the compound, tautomer, stereoisomer or mixture of stereoisomers, pharmaceutically acceptable salt, hydrate, deuterated derivative, or N-oxide thereof, there is a therapeutic effect associated with a reduction in anti-dsDNA antibodies.
51. The method of any one of the preceding embodiments, wherein there is a reduction in one or more inflammatory tissue biomarkers.
52. The method of any one of the preceding embodiments, wherein there is a reduction in severity of the disease or disorder.
53. The method of any one of the preceding embodiments, wherein the disease or disorder is a skin disorder.
54. The method of any one of the preceding embodiments, wherein the disease or disorder is psoriasis.
55. The method of any one of the preceding embodiments, wherein, upon administration of the therapeutically effective amount of the compound, tautomer, stereoisomer or mixture of stereoisomers, pharmaceutically acceptable salt, hydrate, deuterated derivative, or N-oxide thereof, there is a therapeutic effect associated with a reduction in inflammation.
56. The method of any one of the preceding embodiments, wherein the reduction in inflammation comprises a reduction in pathology.
57. The method of any one of the preceding embodiments, wherein the reduction in pathology comprises a reduction in a psoriasis area and severity index score.
58. The method of any one of the preceding embodiments wherein the reduction follows and/or is accompanied by a reduction in inflammatory tissue biomarkers.
59. The method of any one of the preceding embodiments, wherein the inflammatory tissue biomarkers are associated with psoriasis.
60. The method of any one of the preceding embodiments, wherein the inflammatory tissue biomarkers include one or more of IL-17, IL-22, IL-1β, IL-6, TNF-α, and IL-23.
61. A method for the treatment of a skin disease, the method comprising orally administering to a subject in need thereof a therapeutically effective amount of a compound that is:

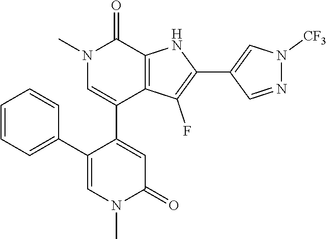 [SMILES: Cn1cc(-c2ccccc2)c(-c2cn(C)c(=O)c3[nH]c(-c4cnn(C(F)(F)F)c4)c(F)c23)cc1=O]

or a tautomer, stereoisomer or mixture of stereoisomers, pharmaceutically acceptable salt, hydrate, deuterated derivative, or N-oxide thereof.
62. A method for the treatment of psoriasis, the method comprising orally administering to a subject in need thereof a therapeutically effective amount of a compound that is:

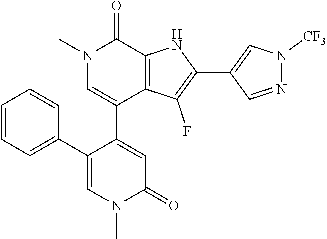 [SMILES: Cn1cc(-c2ccccc2)c(-c2cn(C)c(=O)c3[nH]c(-c4cnn(C(F)(F)F)c4)c(F)c23)cc1=O]

or a tautomer, stereoisomer or mixture of stereoisomers, pharmaceutically acceptable salt, hydrate, deuterated derivative, or N-oxide thereof, wherein, upon administration of the therapeutically effective amount of the compound, tautomer, stereoisomer or mixture of stereoisomers, pharmaceutically acceptable salt, hydrate, deuterated derivative, or N-oxide thereof, a therapeutic effect is associated with a reduction in psoriasis.
63. The method of any one of the preceding embodiments, wherein the compound is

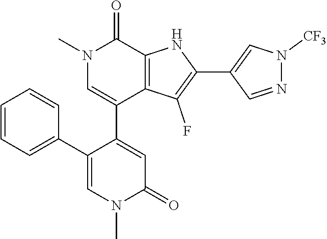 [SMILES: Cn1cc(-c2ccccc2)c(-c2cn(C)c(=O)c3[nH]c(-c4cnn(C(F)(F)F)c4)c(F)c23)cc1=O]

or a pharmaceutically acceptable salt thereof.
64. The method of any one of the preceding embodiments, wherein the progression of psoriasis is slowed or retarded.
65. The method of any one of the preceding embodiments, wherein the appearance or increase of one or more indicators of psoriasis severity is slowed or retarded.
66. A method for the treatment of an inflammatory disease, the method comprising orally administering to a subject in need thereof a therapeutically effective amount of a compound that is:

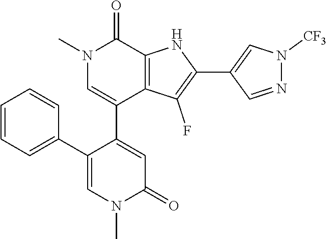 [SMILES: Cn1cc(-c2ccccc2)c(-c2cn(C)c(=O)c3[nH]c(-c4cnn(C(F)(F)F)c4)c(F)c23)cc1=O]

or a tautomer, stereoisomer or mixture of stereoisomers, pharmaceutically acceptable salt, hydrate, deuterated derivative, or N-oxide thereof, wherein, upon administration of the therapeutically effective amount of the compound, tautomer, stereoisomer or mixture of stereoisomers, pharmaceutically acceptable salt, hydrate, deuterated derivative, or N-oxide thereof, a therapeutic effect is associated with a reduction of IL-17A and/or IL-22, and wherein the IC50 thereof is more than about 7,000-fold or more than about 10,000-fold less or between about 7,000 less to about 17,000 less than the IC50 for CXCL10.
67. The method of any one of the preceding embodiments, wherein the disease or disorder is a CNS disease or disorder.
68. The method of any one of the preceding embodiments, wherein the CNS disease or disorder is an autoimmune disease that attacks the central nervous system (CNS), a CNS inflammation, and/or a demyelinating disease.
69. The method of any one of the preceding embodiments, wherein the demyelinating disease is multiple sclerosis (MS) and/or a MS-related condition.
70. The method of any one of the preceding embodiments, wherein the severity of the CNS disease or disorder is reduced.
71. The method of any one of the preceding embodiments, wherein levels of one or more inflammatory biomarkers are reduced.
72. The method of any one of the preceding embodiments, wherein the one or more inflammatory biomarkers are associated with a CNS disease or disorder.
73. The method of any one of the preceding embodiments, wherein the one or more inflammatory biomarkers are associated with multiple sclerosis (MS) and/or a MS-related condition.
74. The method of any one of the preceding embodiments, wherein the one or more inflammatory biomarkers include one or more of IFNγ and IL-12/IL-23p40.
75. The method of any one of the preceding embodiments, wherein CNS inflammation is suppressed.
76. The method of any one of the preceding embodiments, wherein the progression of CNS inflammation is limited, slowed, and/or retarded.
77. The method of any one of the preceding embodiments, wherein the progression of demyelination is limited, slowed, and/or retarded.
78. The method of any one of the preceding embodiments, wherein the progression of autoimmune diseases that attack the CNS is limited, slowed, and/or retarded.
79. The method of any one of the preceding embodiments, wherein the disease course is reversed (e.g., in part) facilitating a recovery. 80. The method of any one of the preceding embodiments, wherein immune cell infiltration into the CNS is modulated.
81. The method of any one of the preceding embodiments, wherein the severity of axonal damage is reduced.
82. The method of any one of the preceding embodiments, wherein the progression of axonal damage is limited, slowed and/or retarded.
83. A method for the treatment of multiple sclerosis, the method comprising orally administering to a subject in need thereof a therapeutically effective amount of a compound selected from:

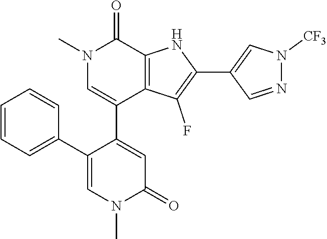 [SMILES: Cn1cc(-c2ccccc2)c(-c2cn(C)c(=O)c3[nH]c(-c4cnn(C(F)(F)F)c4)c(F)c23)cc1=O]

or tautomers, stereoisomers or mixture of stereoisomers, pharmaceutically acceptable salts, hydrates, deuterated derivatives, or N-oxides thereof.
84. The method of any one of the preceding embodiments, wherein progression of multiple sclerosis is slowed or retarded.
85. The method of any one of the preceding embodiments, wherein the compound or a tautomer, stereoisomer or mixture of stereoisomers, pharmaceutically acceptable salt, hydrate, deuterated derivative or an N-oxide thereof is in the form of a pharmaceutical composition which comprises the compound of formula (XXI), tautomer, stereoisomer or mixture of stereoisomers, pharmaceutically acceptable salt, hydrate, deuterated derivative, or N-oxide thereof, and a pharmaceutically acceptable carrier.
86. The method of any one of the preceding embodiments, wherein the compound, tautomer, stereoisomer or mixture of stereoisomers, pharmaceutically acceptable salt, hydrate, deuterated derivative, or N-oxide thereof is formulated as a suspension or partial suspension in the composition.
87. The method of any one of the preceding embodiments, wherein the compound, tautomer, stereoisomer or mixture of stereoisomers, pharmaceutically acceptable salt, hydrate, deuterated derivative, or N-oxide thereof is micronized.
88. The method of any one of the preceding embodiments, wherein the compound, tautomer, stereoisomer or mixture of stereoisomers, pharmaceutically acceptable salt, hydrate, deuterated derivative, or N-oxide thereof comprises nanoparticles.
89. The method of any one of the preceding embodiments, wherein the compound, tautomer, stereoisomer or mixture of stereoisomers, pharmaceutically acceptable salt, hydrate, deuterated derivative, or N-oxide thereof is solubilized or partially solubilized in the composition.
90. The method of any one of the preceding embodiments, wherein the compound, tautomer, stereoisomer or mixture of stereoisomers, pharmaceutically acceptable salt, hydrate, deuterated derivative, or N-oxide thereof or pharmaceutical composition is administered locally, topically, or systemically.
91. The method of any one of the preceding embodiments, wherein the compound, tautomer, stereoisomer or mixture of stereoisomers, pharmaceutically acceptable salt, hydrate, deuterated derivative, or N-oxide thereof or pharmaceutical composition is administered by injection or infusion.
92. The method of any one of the preceding embodiments, wherein the compound, tautomer, stereoisomer or mixture of stereoisomers, pharmaceutically acceptable salt, hydrate, deuterated derivative, or N-oxide thereof or pharmaceutical composition is administered orally.
93. The method of any one of the preceding embodiments, wherein the compound, tautomer, stereoisomer or mixture of stereoisomers, pharmaceutically acceptable salt, hydrate, deuterated derivative, or N-oxide thereof or pharmaceutical composition has activity against one or more BET domains.

This disclosure additionally includes the following numbered embodiments:
1. A compound of formula (XI), or a pharmaceutically acceptable salt or N-oxide thereof:

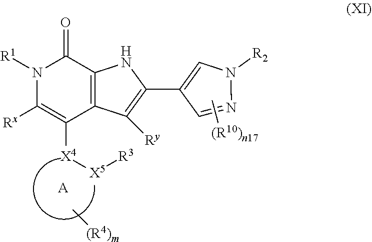

wherein:
Ring A is independently selected from phenyl, 5-membered heterocyclyl and 6-membered heterocyclyl, wherein X⁴ is independently selected from carbon and nitrogen and X⁵ is independently selected from carbon and nitrogen;
R₁ is independently selected from C₁-C₃-alkyl, C₁-C₃-fluoroalkyl, C₃-C₄-cycloalkyl and 4-membered heterocycloalkyl;
R₂ is C₁-C₄-haloalkyl, ethyl, cyano, nitro, isopropyl, tert-butyl, cyclopropyl, and SF₅;
R³ is independently selected from R^{3a}, OR^{3b}, and NR⁶R^{3b};
R^{3a} is independently selected from H, CN, C₁-C₄-alkyl, C₂-C₄-alkenyl, C₂-C₄-alkynyl, C₁-C₄-haloalkyl, C₂-C₄-haloalkenyl, and C₀-C₃-alkylene-R^{3c}; wherein R^{3c} is independently at each occurrence selected from C₃-C₈-cycloalkyl, Cs—Ca-cycloalkenyl, 5- to 8-membered heterocycloalkenyl, 3- to 8-membered heterocycloalkyl, phenyl and 5- or 6-membered heteroaryl; wherein where R^{3c} is cycloalkyl, heterocycloalkyl, cycloalkenyl, or heterocycloalkenyl, R^{3c} is optionally substituted with from 1 to 4 R³ groups and where R^{3c} is phenyl or heteroaryl, R^{3c} is optionally substituted with from 1 to 5 R⁹ groups;
R^{3b} is independently selected from C₁-C₄-alkyl, C₂-C₄-alkylene-O—C₁-C₄-alkyl, C₁-C₄-haloalkyl and C₀-C₃-alkylene-R^{3d}; wherein R^{3d} is independently at each occurrence selected from C₃-C₃-cycloalkyl, 3- to 8-membered heterocycloalkyl, phenyl and 5- or 6-membered heteroaryl; wherein where R^{3d} is cycloalkyl or heterocycloalkyl, R^{3d} is optionally substituted with from 1 to 4 R⁸ groups and where R^{3d} is phenyl or heteroaryl, R^{3d} is optionally substituted with from 1 to 5 R⁹ groups;
R⁴ is independently at each occurrence selected from ═O, ═S, halo, nitro, cyano, C₀-C₄-alkylene-NR⁵R⁶, C₀-C₄-alkylene-OR⁷, SR⁶, SOR⁶, C₀-C₄-alkylene-S(O)₂R⁶, SO₂NR⁶R⁶, C₀-C₄-alkylene-C₂R⁶, C₀-C₄-alkylene-C(O)R⁶, C₀-C₄-alkylene-CONR⁶R⁶, C₁-C₄-alkyl, C₂-C₄-alkenyl, C₂-C₄-alkynyl, C₁-C₄-haloalkyl, C₃-C₆-cycloalkyl, and C₀-C₄-alkylene-R^{4c};
R^{4c} is independently at each occurrence selected from C₃-C₆-cycloalkyl and 4- to 6-membered heterocycloalkyl;
R⁶ is independently at each occurrence selected from H, C₁-C₄-alkyl, C(O)—C₁-C₄-alkyl and S(O)₂—C₁-C₄-alkyl; or R⁵ and R⁶, together with the nitrogen atom to which they are attached form a C₅-C₃-heterocycloalkyl group optionally substituted with from 1 to 4 R⁸ groups;
R⁶ is independently at each occurrence selected from H and C₁-C₄-alkyl; or where two R⁶ groups are attached to the same nitrogen, those two R⁶ groups together with the nitrogen atom to which they are attached optionally form a C₅-C₈-heterocycloalkyl group optionally substituted with from 1 to 4 R⁸ groups:
R⁷ is independently at each occurrence selected from H, C₁-C₄-alkyl, C(O)—C₁-C₄-alkyl and C₁-C₄-haloalkyl;
R⁸ is independently at each occurrence selected from ═O, ═S, fluoro, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl and cyclopropyl;
R⁹ is independently at each occurrence selected from halo, nitro, cyano, C₀-C₄-alkylene-NR⁵R⁶. C₀-C₄-alkylene-OR^{7a}, C₀-C₄-alkylene-SR⁶, C₀-C₄-alkylene-SOR⁶, C₀-C₄-alkylene-S(O)₂R⁶, C₀-C₄-alkylene-SO₂NR⁶R⁶, C₀-C₄-alkylene-CO₂R⁶, C₀-C₄-alkylene-C(O)R, C₀-C₄-alkylene-CONR⁶R⁶, C₀-C₄-alkylene-R^{9a}, C₁-C₄-alkyl, C₂-C₄-alkenyl, C₂-C₄-alkynyl, C₁-C₄-haloalkyl; or where two R⁹ groups are attached to adjacent atoms, those two R⁹ groups together with the atoms to which they are attached optionally form a 5- or 6-membered heterocycloalkyl group optionally substituted with from 1 to 4 R⁸ groups:
R^{7a} is independently at each occurrence selected from H, C₁-C₄-alkyl, C(O)—C₁-C₄-alkyl, C₀-C₄-alkylene-NR⁵R⁶, —C₀-C₄-alkyl-O—R⁷, C₀-C₄-alkylene-SR⁶, C₀-C₄-alkylene-SOR⁶, C₀-C₄-alkylene-S(O)₂R⁶, C₀-C₄-alkylene-SO₂NR⁶R⁶, C₀-C₄-alkylene-CO₂R⁶, C₀-C₄-alkylene-C(O)R⁶, C₀-C₄-alkylene-CONR⁶R⁶ and C₁-C₄-haloalkyl;
R^{9a} is independently at each occurrence selected from C₃-C₆-cycloalkyl and 4- to 6-membered heterocycloalkyl;
R¹⁰ is independently at each occurrence selected from halo, C₁-C₄-alkyl, C₁-C₄-haloalkyl, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl and 4-membered heterocycloalkyl;
R^{x} and R^{y} are each independently selected from H, halo, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl, C₃-C₄-cycloalkyl and 4-membered heterocycloalkyl;
m is an integer selected from 0, 1, 2, 3 and 4;
n₁₇ is an integer selected from 0, 1 and 2;
wherein any of the aforementioned alkyl, alkylene, alkenyl, cycloalkyl or heterocycloalkyl groups is optionally substituted, where chemically possible, by 1 to 5 substituents which are each independently at each occurrence selected from the group consisting of: C₁-C₄-alkyl, oxo, fluoro, nitro, cyano, NR^{a}R^{b}, OR^{a}, SR^{a}, CO₂R^{a}, C(O)R³, CONR^{a}R^{a}, S(O)R^{a} and S(O)₂R^{a}; wherein R^{a} is independently at each occurrence selected from H, C₁-C₄-alkyl and C₁-C₄-haloalkyl; and R^{b} is independently at each occurrence selected from H, C₁-C₄-alkyl, C(O)—C₁-C₄-alkyl and S(O)₂—C₁-C₄-alkyl.
2. A compound of embodiment 1, wherein R² is C₁-C₄-haloalkyl.
3. A compound of embodiment 1 or embodiment 2 wherein R^{x} is H.
4. A compound of any one of embodiments 1 to 3, wherein X⁴ is carbon.
5. A compound of any one of embodiments 1 to 4, wherein R¹ is selected from methyl and ethyl.
6. A compound of any one of embodiments 1 to 5, wherein R² is CF₃.
7. A compound of any one of embodiments 1 to 6, wherein n₁₇ is 0.
8. A compound of any one of embodiments 1 to 7, wherein Ring A is pyridone.
9. A compound of embodiment 8, wherein Ring A is substituted on the nitrogen with 1 group selected from H, C₁-C₄-alkyl, cyclopropyl, cyclobutyl, methyl-cyclobutyl and 4-membered heterocycloalkyl.
10. A compound of embodiment 9, wherein Ring A is

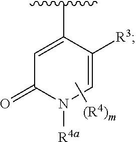

wherein R^{4a} is selected from H, C₁-C₄-alkyl, cyclopropyl and 4-membered heterocycloalkyl.
11. A compound of embodiment 10, wherein R^{4a} is selected from methyl, cyclopropyl, oxetane, —CH₂—CH₂—OMe and azetidine.
12. A compound of any one of embodiments 1 to 11, wherein R³ is R^{3a}.
13. A compound of embodiment 12, wherein R^{3a} is phenyl optionally substituted with from 1 to 3 R⁹ groups.
14. A compound of any one of embodiments 1 to 13, wherein R^{y} is H.
15. A pharmaceutical composition comprising a compound of any one of embodiments 1 to 14, and one or more pharmaceutically acceptable excipients.
16. A compound of any one of embodiments 1 to 14 for use as a medicament.
17. A compound of any one of embodiments 1 to 14 for use in treating a disease or disorder selected from one or more of an inflammatory disorder, an immune disorder, and an autoimmune disorder.
18. A compound of any one of embodiments 1 to 14 for use in treating a cancer.

This disclosure additionally includes the following numbered embodiments:
1. A method for the treatment of an inflammatory and/or an autoimmune disease or disorder or a disease or disorder related thereto, the method comprising administering to a subject in need thereof a therapeutically effective amount of a compound of formula (XXIA), a tautomer, a stereoisomer or a mixture of stereoisomers, a pharmaceutically acceptable salt, a hydrate, a deuterated derivative, or an N-oxide thereof:

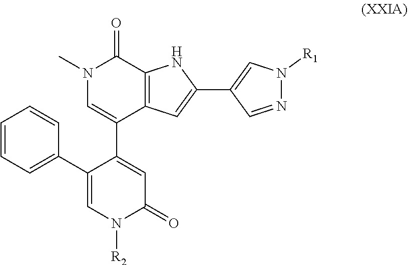

wherein:
R₁ is C₁-C₄-haloalkyl;
R₂ is C₁-C₄-alkyl, wherein the C₁-C₄-alkyl is optionally substituted with SR₃ or OR₃; and
R₃ is selected from H, C₁-C₃-alkyl, C(O)—C₁-C₃-alkyl, and C₁-C₃-haloalkyl.
2. The method of embodiment 1, wherein R₁ is independently selected from CF₃, CHF₂, CH₂CF₃, and CH₂CH₂F.
3. The method of embodiment 1, wherein R₁ is independently selected from CBr₃, CHBr₂, CH₂CBr₃, and CH₂CH₂Br.
4. The method of embodiment 1, wherein R₁ is independently selected from CCl₃, CHCl₂, CH₂CCl₃, and CH₂CH₂Cl.
5. The method of embodiment 1, wherein R₁ is CH₂CH₂F.
6. The method of embodiment 1, wherein R₁ is CHF₂.
7. The method of embodiment 1, wherein R₁ is CF₃.
8. The method of embodiment 1, wherein R₂ is C₁-C₄-alkyl, wherein the C₁-C₄-alkyl is substituted with OR₃.
9. The method of embodiment 1, wherein R₂ is C₁-C₄-alkyl, wherein the C₁-C₄-alkyl is substituted with SR₃.
10. The method of embodiment 8, wherein R₂ is selected from —CH₃, —CH₂—CH₂—O—CH₃, and —CH₂—CH₂—OCH₂—CH₃.
11. The method of embodiment 9, wherein R₂ is selected from —CH₃, —CH₂—CH₂—S—CH₃, and —CH₂—CH₂—SCH₂—CH₃.
12. The method of embodiment 1, 10 or 11, wherein R₂ is —CH₃.
13. The method of embodiment 1 or 10, wherein R₂ is —CH₂—CH₂—O—CH₃.
14. The method of embodiment 1 or 11, wherein R₂ is —CH₂—CH₂—S—CH₃.
15. The method of embodiment 1, wherein the compound of formula (XXIA) is selected from:

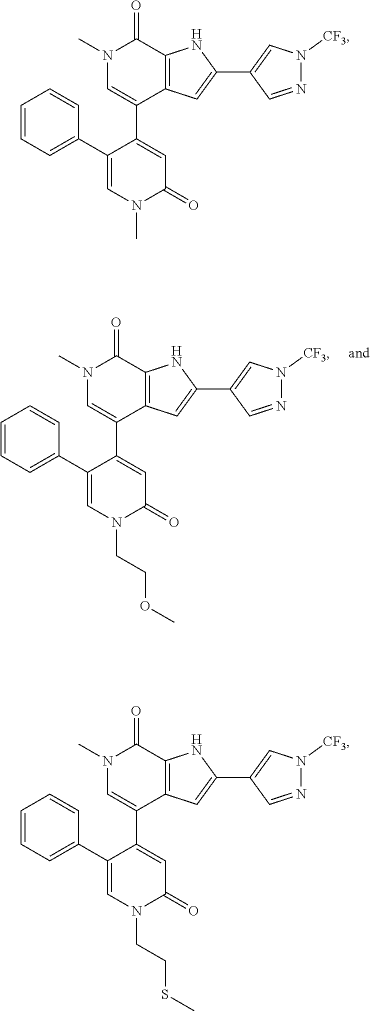 [SMILES: Cn1cc(-c2ccccc2)c(-c2cn(C)c(=O)c3[nH]c(-c4cnn(C(F)(F)F)c4)cc23)cc1=O ; COCCn1cc(-c2ccccc2)c(-c2cn(C)c(=O)c3[nH]c(-c4cnn(C(F)(F)F)c4)cc23)cc1=O ; "and" ; CSCCn1cc(-c2ccccc2)c(-c2cn(C)c(=O)c3[nH]c(-c4cnn(C(F)(F)F)c4)cc23)cc1=O]

or a tautomer, a stereoisomer or mixture of stereoisomers, a pharmaceutically acceptable salt, a hydrate, a deuterated derivative, or N-oxide thereof.
16. The method of embodiment 1, wherein the compound of formula (XXIA) is

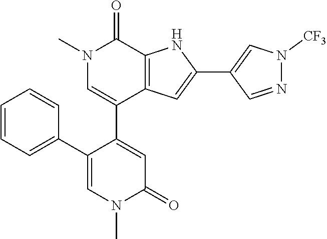 [SMILES: Cn1cc(-c2ccccc2)c(-c2cn(C)c(=O)c3[nH]c(-c4cnn(C(F)(F)F)c4)cc23)cc1=O]

or a tautomer, a stereoisomer or mixture of stereoisomers, a pharmaceutically acceptable salt, a hydrate, a deuterated derivative, or N-oxide thereof.
17. The method of embodiment 1, wherein the compound of formula (XXIA) is

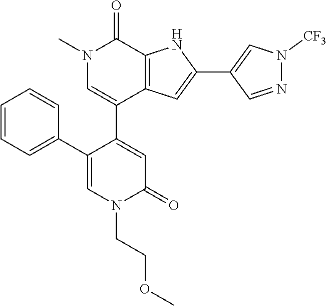 [SMILES: COCCn1cc(-c2ccccc2)c(-c2cn(C)c(=O)c3[nH]c(-c4cnn(C(F)(F)F)c4)cc23)cc1=O]

or a tautomer, a stereoisomer or mixture of stereoisomers, a pharmaceutically acceptable salt, a hydrate, a deuterated derivative, or N-oxide thereof.
18. The method of embodiment 1, wherein the compound of formula (XXIA) is

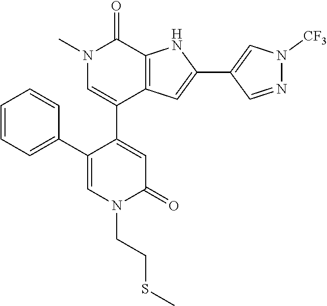 [SMILES: CSCCn1cc(-c2ccccc2)c(-c2cn(C)c(=O)c3[nH]c(-c4cnn(C(F)(F)F)c4)cc23)cc1=O]

or a tautomer, a stereoisomer or mixture of stereoisomers, a pharmaceutically acceptable salt, a hydrate, a deuterated derivative, or N-oxide thereof.
19. A method for the treatment of an inflammatory and/or an autoimmune disease or disorder or a disease or disorder related thereto, the method comprising administering to a subject in need thereof a therapeutically effective amount of a compound of formula (XXI), a tautomer, a stereoisomer or a mixture of stereoisomers, a pharmaceutically acceptable salt, a hydrate, a deuterated derivative, or an N-oxide thereof:

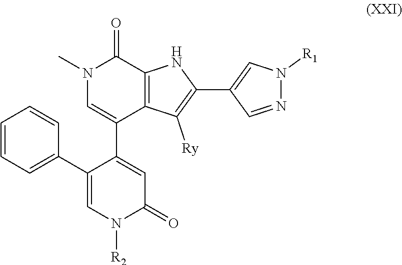

wherein:
R₁ is C₁-C₄-haloalkyl;
R₂ is C₁-C₄-alkyl, wherein the C₁-C₄-alkyl is optionally substituted with SR₃ or OR₃;
R₃ is selected from H, C₁-C₃-alkyl, C(O)—C₁-C₃-alkyl, and C₁-C₃-haloalkyl; and
R_{y} is hydrogen or halo.
20. The method of embodiment 19, wherein R₁ is independently selected from CF₃, CHF₂, CH₂CF₃, and CH₂CH₂F.
21. The method of embodiment 19, wherein R₁ is independently selected from CBr₃, CHBr₂, CH₂CBr₃, and CH₂CH₂Br.
22. The method of embodiment 19, wherein R₁ is independently selected from CCl₃, CHCl₂, CH₂CCl₃, and CH₂CH₂Cl.
23. The method of embodiment 19, wherein R₁ is CH₂CH₂F.
24. The method of embodiment 19, wherein R₁ is CHF₂.
25. The method of embodiment 19, wherein R₁ is CF₃.
26. The method of any one of embodiments 19-25, wherein R₂ is —CH₃.
27. The method of any one of embodiments 19-25, wherein R_{y} is halo.
28. The method of embodiment 27, wherein R_{y} is fluorine.
29. The method of embodiment 19, wherein the compound of formula (XXI) is:

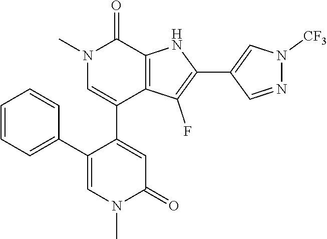 [SMILES: Cn1cc(-c2ccccc2)c(-c2cn(C)c(=O)c3[nH]c(-c4cnn(C(F)(F)F)c4)c(F)c23)cc1=O]

or a tautomer, a stereoisomer or mixture of stereoisomers, a pharmaceutically acceptable salt, a hydrate, a deuterated derivative, or N-oxide thereof.
30. A pharmaceutical composition comprising a compound of any one of embodiments 1, 15-19, or 29, or a pharmaceutically acceptable salt thereof, or N-oxide thereof, and one or more pharmaceutically acceptable excipients.
31. A method of treating a disease or disorder selected from one or more of an inflammatory disease or disorder, an immune disease or disorder, and an autoimmune disease or disorder, comprising administering to a warm-blooded animal a therapeutically effective amount of a compound of any one of embodiments 1, 15-19, or 29, or a pharmaceutically acceptable salt thereof, or a N-oxide thereof.
32. The method of treatment of embodiment 31, wherein the disease or disorder is a joint disease or disorder or a joint-related disease or disorder.
33. The method of treatment of embodiment 31 or 32, wherein the disease or disorder is selected from arthritis, bursitis, Ehlers-Danlos syndrome, epicondylitis, Felty Syndrome, gouty arthritis, psoriatic arthritis, osteoarthritis, rheumatoid arthritis, Still's disease, tenosynovitis, synovitis, Sjögren's Syndrome, Lyme disease, Whipple disease, bone cancer, lupus, and other autoimmune joint disorders.
34. The method of treatment of embodiment 32, wherein the joint disease or disorder or the joint-related disease or disorder is arthritis.
35. The method of treatment of embodiment 34, wherein the arthritis is rheumatoid arthritis.
36. The method of treatment of embodiment 31, wherein the disease or disorder is a fibrotic disease or disorder.
37. The method of treatment of embodiment 31 or 36, wherein the disease or disorder is renal fibrosis.
38. The method of treatment of embodiment 31 or 36, wherein the disease or disorder is pulmonary fibrosis.
39. The method of treatment of embodiment 31, wherein the disease or disorder is a skin disease or disorder.
40. The method of treatment of embodiment 31 or 39, wherein the disease or disorder is psoriasis.
41. The method of treatment of embodiment 31, wherein the disease or disorder is a lupus disease or disorder.
42. The method of treatment of embodiment 31 wherein the disease or disorder is a MS or MS related disease or disorder.
43. The method of treatment of any one of embodiments 31-42, wherein the severity of the disease or disorder is reduced.

### EXAMPLES

### Abbreviations

| | |
|---|---|
| BD | binding domain |
| BET | bromodomain and extra terminal domain |
| br | broad |
| BRD | Bromodomain-containing protein |
| BRET | Bioluminescence resonance energy transfer |
| c | centi |
| δ | delta |
| d | doublet |
| dd | double doublet |
| DCM | dichloromethane |
| Dioxane | 1,4-dioxane |
| DMF | dimethyl formamide |
| DMA | dimethyl acetamide |
| DMSO | dimethyl sulfoxide |
| EA | Ethyl acetate |
| EC | effective concentration |
| ES | electrospray |
| ESI | electrospray ionization |
| FA | formic acid |
| g | gram |
| hr | hour |
| HPLC | high performance liquid chromatography |
| HRMS | high resolution mass spectrum |
| J | coupling constant |
| K_{d} | binding affinity |
| L | litre |
| LC | liquid chromatography |
| LG | leaving group |
| m | multiplet |
| m | milli |
| m | meter |
| M | molar |
| M+ | molecular ion |
| MHz | megahertz |
| min | minutes |
| mol | mole |
| MS | mass spectrometry |
| m/z | mass/charge |
| n | nano |
| NMR | nuclear magnetic resonance |
| PE | petroleum ether |
| p | para |
| PTSA | p-Toluenesulfonic acid |
| q | quartet |
| Rf | retardation factor |
| rpm | revolutions per minute |
| RT | room temperature |
| s | singlet |
| SM | starting material |
| S_{N}Ar | nucleophilic aromatic substitution (addition- |
| | elimination) |
| t | triplet |
| Tetrakis | Pd(PPh₃)₄ |
| TFA | Trifluoroacetic acid |
| THF | tetrahydrofuran |
| TLC | thin layer chromatography |
| TBME | methyl tert-butyl ether |
| t_{R} | retention time |
| XPhos | 2-dicyclohexylphosphino-2′,4′,6′-triisopropylbiphenyl |
| Ts or Tosyl | toluenesulfonyl |
| μ | micro |
| UPLC | ultra performance liquid chromatography |
| UV | ultraviolet |
| vis | visible |
| w/w | weight by weight |
| ° C. | degree Celsius |
| % | per cent |

### Analytical Methods:

### NMR MOA

¹H NMR spectra are recorded on Bruker AVANCE III HD 300, Bruker AVANCE NEO 400 or Bruker AVANCE III HD 400 spectrometers.

Chemical shifts are denoted in ppm (δ) relative to residual protonated solvent as an internal standard as described in, for example, Gottlieb et al., J. of Org. Chem, 62(21):7512-15 (1997). The splitting pattern for NMR spectra are denoted as follows: s (singlet), br (broad), d (doublet), t (triplet), m (multiplet) or combinations thereof. Coupling constants (J) are designated in Hz and reported to one decimal place.

Liquid chromatography-mass spectra (LCMS) are recorded using the following systems and running conditions:

| | | | Initial | Middle | Gradient | Final | Gradient | | Flow | |
|---|---|---|---|---|---|---|---|---|---|---|
| | | | Water | Water | Length | Water | Length | Held | Rate | Con- |
| Instrument | Column | Mobile Phase | (%) | (%) | (min) | (%) | (min) | (min) | (mL/min) | dition |
| Shimadzu | Polar-C18 | Water | 90 | 30 | 1.80 | 0 | 0.20 | 0.70 | 1.50 | A |
| LCMS-2020 | 30 mm × | (0.1% FA)/ | | | | | | | | |
| Shimadzu | 2.1 mm, | CH₃CN(0.1% | 100 | — | — | 0 | 2.00 | 0.70 | 1.50 | AF |
| LCMS-2020 | 2.6 μm | FA) | | | | | | | | |
| | Express- | Water | 100 | 40 | 1.90 | 0 | 0.30 | 0.6 | 1.80 | B |
| | C18 | (0.1% FA)/ | | | | | | | | |
| | 30 mm X | CH₃CN | | | | | | | | |
| | 3.0 mm, 2.6 | (0.1% FA) | | | | | | | | |
| | μm | | | | | | | | | |
| | EVO-C18 | Water | 90 | 30 | 2.00 | 5 | 0.20 | 0.40 | 1.20 | C |
| | 50 mm × | (0.04% | 90 | 30 | 2.00 | 5 | 0.20 | 0.40 | 1.50 | H |
| | 3.0 mm, 2.6 | NH₃•H₂O)/ | 90 | / | / | 5 | 2.00 | 0.60 | 1.20 | J |
| | μm | Acetonitrile | 80 | 30 | 2.00 | 5 | 0.20 | 0.65 | 1.50 | O |
| | | | 80 | 30 | 1.85 | 5 | 0.25 | 0.50 | 1.50 | Q |
| | | | 100 | 40 | 2.20 | 5 | 0.20 | 0.40 | 1.50 | T |
| | | | 100 | 30 | 2.00 | 5 | 0.20 | 0.40 | 1.50 | AA |
| | | | 100 | 50 | 2.20 | 5 | 0.20 | 0.40 | 1.50 | BB |
| | | Water | 90 | 30 | 2.00 | 5 | 0.20 | 0.40 | 1.50 | BA |
| | | (5 mM | | | | | | | | |
| | | NH₄HCO₃)/ | | | | | | | | |
| | | Acetonitrile | | | | | | | | |
| | | | 80 | 30 | 1.85 | 5 | 0.25 | 0.50 | 1.50 | BC |
| | EC-C18 | Water | 100 | 40 | 2.20 | 0 | 0.5 | 0.3 | 1.50 | D |
| | 30 × 3 mm, | (0.1% FA)/ | 80 | 30 | 2.00 | 5 | 0 | 0.54 | 1.50 | P |
| | 2.7 μm | CH₃CN | 95 | 30 | 2.00 | 0 | 0.30 | 0.44 | 1.50 | R |
| | | (0.1% FA) | | | | | | | | |
| | Ascentis | Water | 100 | 50 | 1.90 | 0 | 0.35 | 0.35 | 1.50 | E |
| | Express | (0.05% TFA)/ | 100 | 60 | 2.00 | 0 | 0.2 | 0.4 | 1.50 | U |
| | C18 | MeCN | 100 | 40 | 1.90 | 0 | 0.35 | 0.35 | 1.50 | W |
| | 30 mm X | (0.05% TFA) | 100 | 30 | 2 | 0 | 0.1 | 0.5 | 1.50 | BD |
| | 3.0 mm, 2.7 | | | | | | | | | |
| | μm | | | | | | | | | |
| | HPH C18 | Water | 80 | 20 | 2.80 | 5 | 0.20 | 0.50 | 1.50 | F |
| | 50 mm X | (0.04% | 80 | 30 | 2.00 | 5 | 0.20 | 0.50 | 1.50 | K |
| | 3.0 mm, 4.0 | NH₃H₂O)/ | 100 | 30 | 2.20 | 5 | 0.20 | 0.40 | 1.50 | L |
| | μm | CH₃CN | 100 | 30 | 2.00 | 5 | 0.20 | 0.40 | 1.50 | Z |
| | | | 100 | 40 | 2.00 | 5 | 0.40 | 0.40 | 1.50 | AD |
| | | Water | 80 | 30 | 2.0 | 0 | 0.2 | 0.50 | 1.5 | AW |
| | | (0.1% FA)/ | 95 | 30 | 1.80 | 0 | 0.20 | 0.50 | 1.50 | BE |
| | | MeCN | | | | | | | | |
| | | (0.1% FA) | | | | | | | | |
| | HALO-C18 | Water | 95 | 30 | 1.79 | 0 | 0.20 | 0.70 | 1.50 | M |
| | 30 mm × | (0.1% FA)/ | 100 | 30 | 2.20 | 0 | 0.20 | 0.40 | 1.50 | V |
| | 3.0 mm, | MeCN | 100 | 40 | 1.90 | 0 | 0.35 | 0.35 | 1.50 | W |
| | 2.0 μm | (0.1% FA) | 100 | 40 | 1.80 | 0 | 0.20 | 0.70 | 1.50 | X |
| | | | 100 | 30 | 1.80 | 0 | 0.20 | 0.70 | 1.50 | Y |
| | | | 70 | 20 | 2.30 | 0 | 0.20 | 0.20 | 1.50 | AR |
| | | | 95 | 30 | 2.00 | 0 | 0.30 | 0.44 | 1.50 | AT |
| | | | 100 | 40 | 2.20 | 0 | 0.30 | 0.30 | 1.50 | AU |
| | | | 95 | 30 | 1.80 | 0 | 0.2 | 0.70 | 1.50 | AV |
| | Poroshell | Water/5 mM | 100 | 30 | 2.00 | 5 | 0.20 | 0.50 | 1.50 | N |
| | HPH-C18 | NH₄HCO₃/ | | | | | | | | |
| | 50 mm × 3 | MeCN | | | | | | | | |
| | mm, 4.0 μm | | | | | | | | | |
| | XB-C18, | Water | 95 | 30 | 2.00 | 0 | 0.30 | 0.44 | 1.50 | AS |
| | 50 mm × | (0.1% FA)/ | | | | | | | | |
| | 3.0 mm, | MeCN(0.1% FA) | | | | | | | | |
| | 4.0 μm | | | | | | | | | |
| | Poroshell | Water | 0 | 30 | 2.00 | 5 | 0.20 | 0.50 | 1.50 | AT |
| | HP-C18, 50 | (5 mM | | | | | | | | |
| | mm × 3.0 | NH₄HCO₃)/ | | | | | | | | |
| | mm, 2.7 μm | Acetonitrile | | | | | | | | |
| Waters Acquity | Waters X- | Water 0.1% | 97 | | | 0 | 4.0 | 0.50 | 0.8 | XX |
| UPLC | Bridge | FA/Water | | | | | | | | |
| | (50 mm | 0.1% | | | | | | | | |
| | X2.1 mm, | FA:CH₃CN | | | | | | | | |
| | 2.5 μm) | (10:90) | | | | | | | | |

### Purification Methods:

Purification by preparative HPLC (prep-HPLC) employ the following instruments and conditions:

| | | | Initial | Final | Flow | Gradient | |
|---|---|---|---|---|---|---|---|
| | | | Water | Water | Rate | Length | Con- |
| Instrument | Column | Mobile Phase | (%) | (%) | (mL/min) | (min) | dition |
| Biotage Isolera | C18 | MeCN in Water | 70 | 50 | 50 | 10 | A |
| Prime | | (0.1% FA) | 90 | 50 | 50 | 10 | C |
| | | | 100 | 0 | 50 | 10 | E |
| | | | 90 | 50 | 50 | 15 | F |
| | | | 50 | 20 | 50 | 7 | L |
| | | | 70 | 50 | 50 | 10 | M |
| | | | 50 | 5 | 50 | 10 | O |
| | | | 90 | 0 | 50 | 30 | R |
| | | | 90 | 0 | 50 | 35 | T |
| | | | 30 | 10 | 100 | 10 | W |
| | | | 90 | 40 | 100 | 10 | X |
| | | | 90 | 50 | 100 | 15 | Y |
| | | | 60 | 44 | 80 | 15 | Z |
| | | | 90 | 30 | 60 | 30 | AK |
| | | | 70 | 30 | 60 | 30 | AN |
| | | | 80 | 50 | 60 | 10 | AQ |
| | | | 90 | 50 | 60 | 10 | AR |
| | | | 95 | 5 | 60 | 30 | AS |
| | | | 90 | 50 | 60 | 15 | AT |
| | | | 90 | 50 | 60 | 10 | AX |
| | | | 90 | 60 | 60 | 30 | BW |
| | | | 60 | 25 | 60 | 15 | BV |
| | | | 90 | 30 | 60 | 45 | BZ |
| | | | 40 | 10 | 60 | 30 | CA |
| | | | 63 | 58 | 60 | 10 | CE |
| | | Water (0.04% | 70 | 50 | 60 | 10 | AJ |
| | | NH₃•H₂O)/MeCN | 50 | 30 | 60 | 15 | AM |
| | | | 70 | 40 | 60 | 10 | H |
| | | | 90 | 50 | 60 | 30 | BF |
| | | | 95 | 50 | 60 | 20 | CI |
| | | Water (0.1% | 90 | 40 | 60 | 20 | CJ |
| | | NH₃•H₂O)/MeCN | 90 | 50 | 60 | 25 | CL |
| | | | 90 | 5 | 60 | 30 | CO |
| | | | 90 | 50 | 60 | 10 | CQ |
| | CHIRAL ART | Mobile Phase A: Hex(10 | 50 | 50 | 20 | 20 | CS |
| | Amylose-SA | mM NH₃—MeOH), Mobile | | | | | |
| | 2*25 cm, 5 μm | Phase B: IPA | | | | | |
| Waters 2545 Binary | Ultimate XB- | Water (0.1% FA)/ | 95 | 58 | 100 | 16 | B |
| Gradient Module | C18 Column | MeCN | 95 | 55 | 70 | 30 | U |
| with Waters 2489 | 50 × 250 mm, 10 | | 95 | 61 | 100 | 18 | V |
| UV/visible detector | μm | | 70 | 30 | 100 | 30 | AB |
| | | | 80 | 40 | 100 | 30 | BG |
| | | Water (10 mM | 70 | 40 | 100 | 30 | AL |
| | | NH₄HCO₃)/MeCN | 80 | 50 | 100 | 30 | AP |
| | | | 70 | 30 | 100 | 30 | BI |
| | | | 80 | 45 | 100 | 30 | CM |
| | YMC-Actus | Water (0.1% FA)/ | 85 | 55 | 100 | 30 | D |
| | Triart C18 | MeCN | 70 | 40 | 100 | 30 | K |
| | ExRS, 30 × 150 | | 61 | 48 | 60 | 10 | BJ |
| | mm, 5 μm | Water(10 mmol/L | 63 | 54 | 60 | 9 | CB |
| | | NH₄HCO₃ + 0.1% | 80 | 60 | 60 | 10 | CC |
| | | NH₃•H₂O)/CH₃CN | | | | | |
| | | Water(0.05% FA)/MeOH | 50 | 49 | 25 | 10 | BU |
| | | Water(10 mmol/L | 87 | 70 | 60 | 10 | CR |
| | | NH₄HCO₃)/CH₃CN | | | | | |
| | XBridge Prep | Water (0.1% FA)/ | 84 | 66 | 60 | 10 | G |
| | C18 OBD | MeCN | 76 | 59 | 50 | 7 | Q |
| | Column, 19 × | | 87 | 72 | 50 | 8 | BB |
| | 150 mm, 5 μm | | 78 | 68 | 60 | 8.5 | BH |
| | | | 72 | 54 | 60 | 10 | CF |
| | | Water | 100 | 70 | 15 | 40 | I |
| | | (0.04% NH₃•H₂O)/MeCN | 100 | 45 | 60 | 20 | AA |
| | | | 90 | 60 | 100 | 30 | AF |
| | | | 60 | 30 | 100 | 30 | AG |
| | | | 75 | 42 | 100 | 30 | AV |
| | | | 68 | 55 | 50 | 8 | BM |
| | | | 80 | 20 | 60 | 15 | BO |
| | | Water | 75 | 50 | 60 | 10 | BT |
| | | (0.1% NH₃•H₂O)/MeCN | | | | | |
| | | Water | 70 | 50 | 100 | 30 | BC |
| | | (10 mMol NH₃•H₂O)/MeCN | | | | | |
| | | Water(0.1% FA)/MeOH | 44 | 38 | 50 | 8 | BP |
| | | Water(10 mmol/L | 59 | 42 | 60 | 10 | BX |
| | | NH4HCO₃ + 0.1% | | | | | |
| | | NH₃•H₂O)/MeCN | | | | | |
| | XBridge Prep | Water(10 mmol/L | 73 | 58 | 60 | 10 | CG |
| | C18 OBD | NH₄HCO₃ + 0.1% | 74 | 66 | 60 | 10 | CH |
| | Column, 30 × | NH₃•H₂O)/MeCN | | | | | |
| | 150 mm, 5 μm | | | | | | |
| | Xselect CSH | Water(0.1% FA)/MeCN | 75 | 40 | 60 | 9 | J |
| | C18 OBD | | 90 | 90 | 60 | 30 | N |
| | Column 30 × | | 90 | 85 | 60 | 20 | AC |
| | 150 mm 5 μm | | 55 | 25 | 100 | 25 | AD |
| | | | 88 | 76 | 60 | 9 | AU |
| | | Water(0.05% FA)/MeOH | 65 | 42 | 60 | 10 | BR |
| | Sunfire prep | Water (0.1% FA)/MeCN | 70 | 52 | 60 | 10 | Al |
| | C18 colμmn, | | 88 | 35 | 60 | 8 | BE |
| | 30 × 150 mm, 5 | | 73 | 68 | 60 | 8 | CD |
| | μm | | | | | | |
| | XB C18, | Water (0.05% | 95 | 5 | 100 | 30 | BD |
| | 50*250 mm, 10 | NH₄HCO₃)/MeCN | 85 | 45 | 100 | 30 | BN |
| | μm | | 80 | 45 | 100 | 30 | BQ |
| | | | 85 | 35 | 100 | 35 | BY |
| | | | 95 | 5 | 100 | 30 | BS |
| | Ultimate@- | Water (10 mM FA)/MeOH | 80 | 55 | 100 | 35 | CK |
| | Phenyl 50 x | | | | | | |
| | 250 mm, 10 μm | | | | | | |
| | XBridge Prep | Water(10 mmol/L | 78 | 60 | 60 | 10 | CN |
| | Phenyl OBD | NH₄HCO₃ + 0.1% | | | | | |
| | Column, 19 × | NH₃•H₂O)/MeCN | | | | | |
| | 250 mm, 5 μm | | | | | | |
| | XBridge Shield | Water (10 mmol/L | 80 | 50 | 60 | 10 | CP |
| | RP18 OBD | NH₄HCO₃ + 0.1% | | | | | |
| | Column 30*150 | NH₃•H₂O)/MeCN | | | | | |
| | mm, 5 μm | | | | | | |

Purification by SFC employs the following instruments and conditions:

| | | | | | Flow | | |
|---|---|---|---|---|---|---|---|
| | | | Phase A | Phase A | Rate | Hold | |
| Instrument | Column | Mobile Phase | (%) | (%) | (mL/min) | (min) | Condition |
| GILSON- | CHIRAL ART | Phase A: MtBE(10 mM | 85 | 85 | 20 | 7.89 | AE |
| 281 | Amylose-SA | NH₃—MeOH) | | | | | |
| | 2*25 cm, 5 μm | Phase B: EtOH--HPLC | | | | | |
| GILSON- | CHIRAL ART | Phase A: Hex(10 mM | 50 | 50 | 20 | 11.735 | AH |
| 281 | Amylose-SA | Phase B: NH₃—MeOH)/ | | | | | |
| | 2*25 cm, 5 μm | IPA--HPLC | | | | | |

### Process for Preparation

Certain compounds of the disclosure may be synthesised according to the methods disclosed in Scheme 1 and 2 and General Schemes I to 10. Certain compounds of the disclosure may be synthesised according to or analogously to the syntheses provided in examples 1 to 116.

The following schemes illustrate methods of synthesising the compounds of the disclosure.

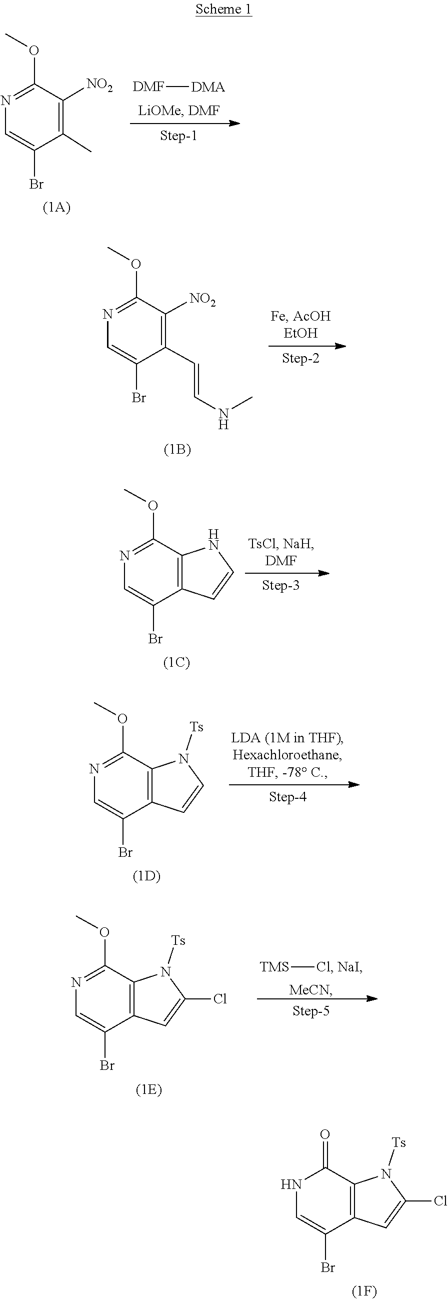

Scheme 1 illustrates the route for the preparation of intermediate (1F) of the disclosure.

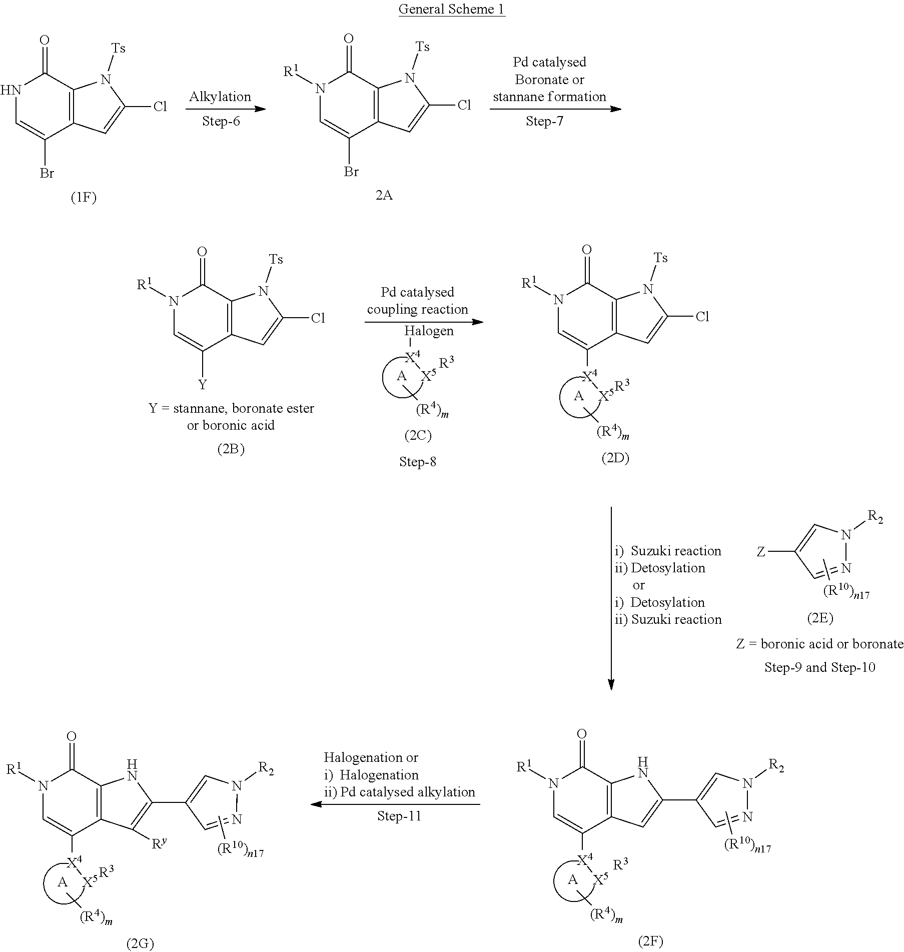

General Scheme 1 illustrates a general route for the preparation of compounds of the disclosure via alkylation of intermediate (1F) followed by Pd catalysed stannane or boronate formation, a Pd catalysed coupling reaction followed by either Suzuki coupling then detosylation or detosylation then Suzuki coupling gave compounds of the disclosure such as (2F). Subsequent halogenation or halogenation followed by Pd catalysed alkylation gives compounds of the disclosure such as (2G).

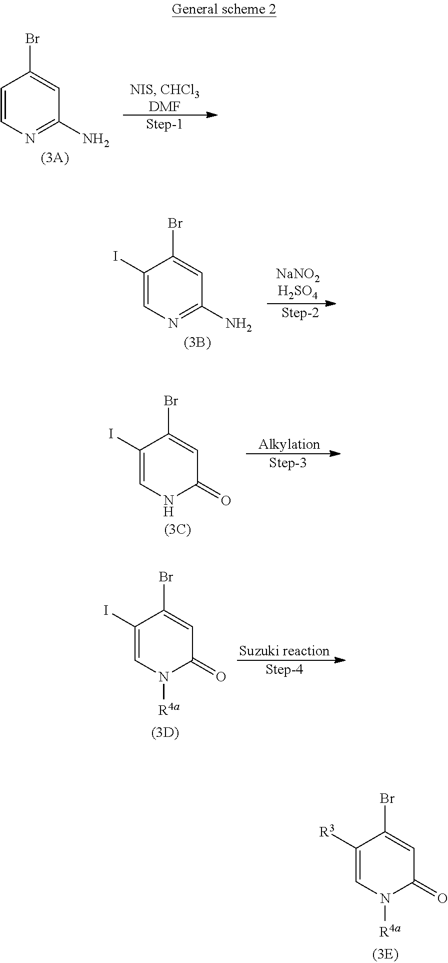

General Scheme 2 illustrates a general route for the preparation of general intermediate (3E) (a subgenus of intermediate (20)) of the disclosure. From (3A) iodination, pyridone formation, alkylation and Suzuki reaction gave (3E).

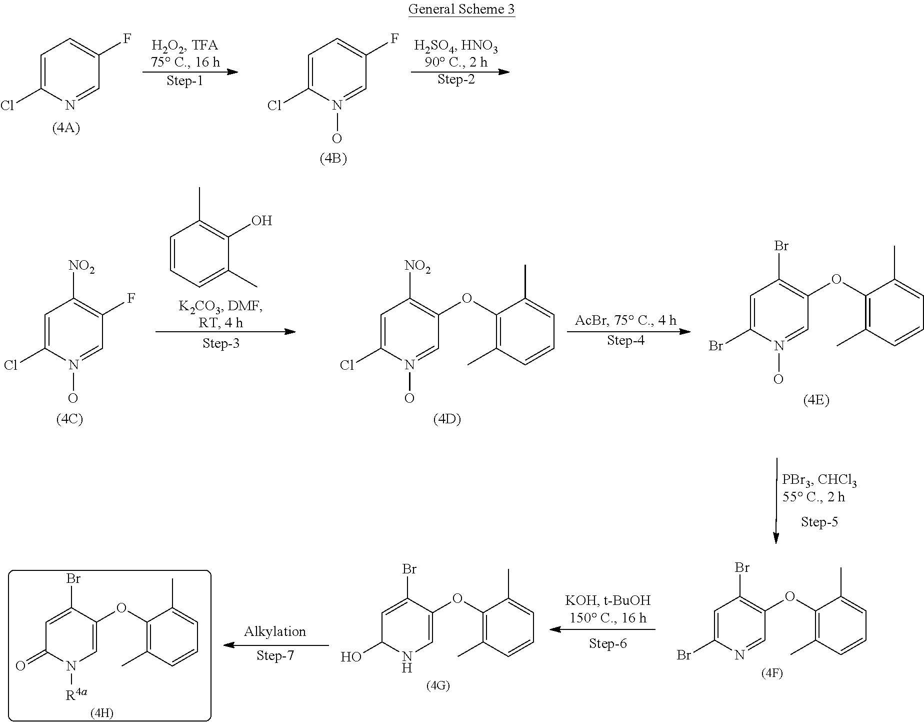

General Scheme 3 illustrates a general route for the preparation of general intermediate (4H) (a subgenus of intermediate (2C)) of the disclosure. From (4A) N-oxide formation, nitration, ether formation, bromination, pyridine formation, pyridone formation and alkylation gave (4H).

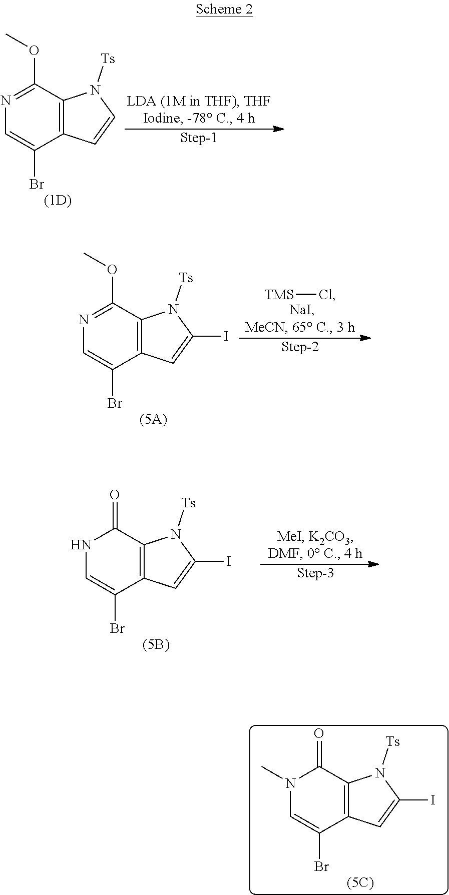 [SMILES: COc1ncc(Br)c2ccn(S(=O)(=O)c3ccc(C)cc3)c12 ; COc1ncc(Br)c2cc(I)n(S(=O)(=O)c3ccc(C)cc3)c12 ; C[Si](C)(C)Cl ; Cc1ccc(S(=O)(=O)n2c(I)cc3c(Br)c[nH]c(=O)c32)cc1 ; Cc1ccc(S(=O)(=O)n2c(I)cc3c(Br)cn(C)c(=O)c32)cc1]

Scheme 2 illustrates the route for the preparation of intermediate (5C) of the disclosure.

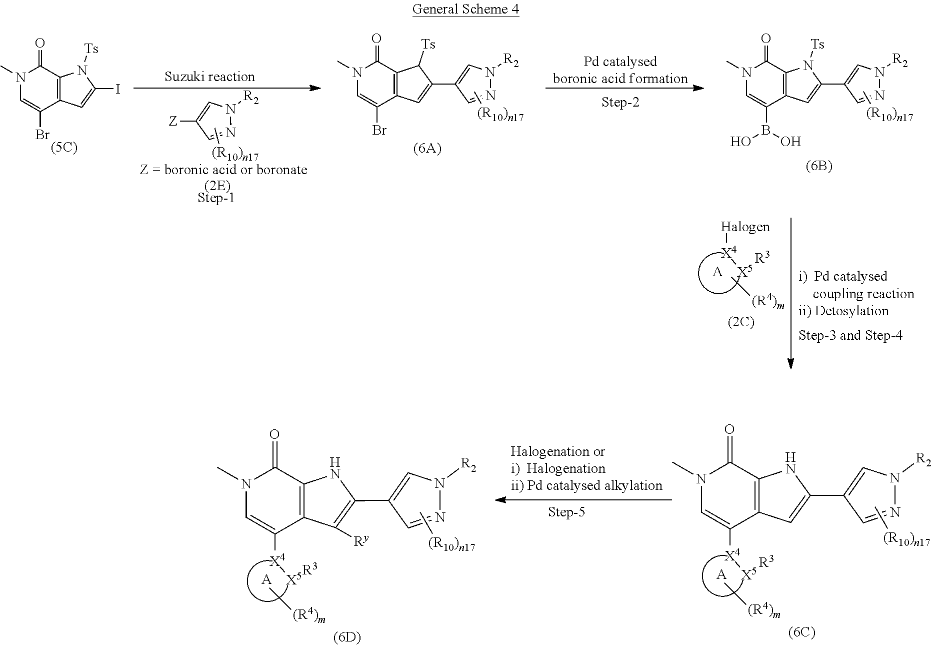

General Scheme 4 illustrates a general route for the preparation of compounds of the disclosure. From (5C) Suzuki coupling followed by boronic acid formation, Pd coupling and detosylation gave compounds of the disclosure such as (6C). Subsequent halogenation or halogenation and the Pd catalysed alkylation gave compounds of the disclosure such as (6D).

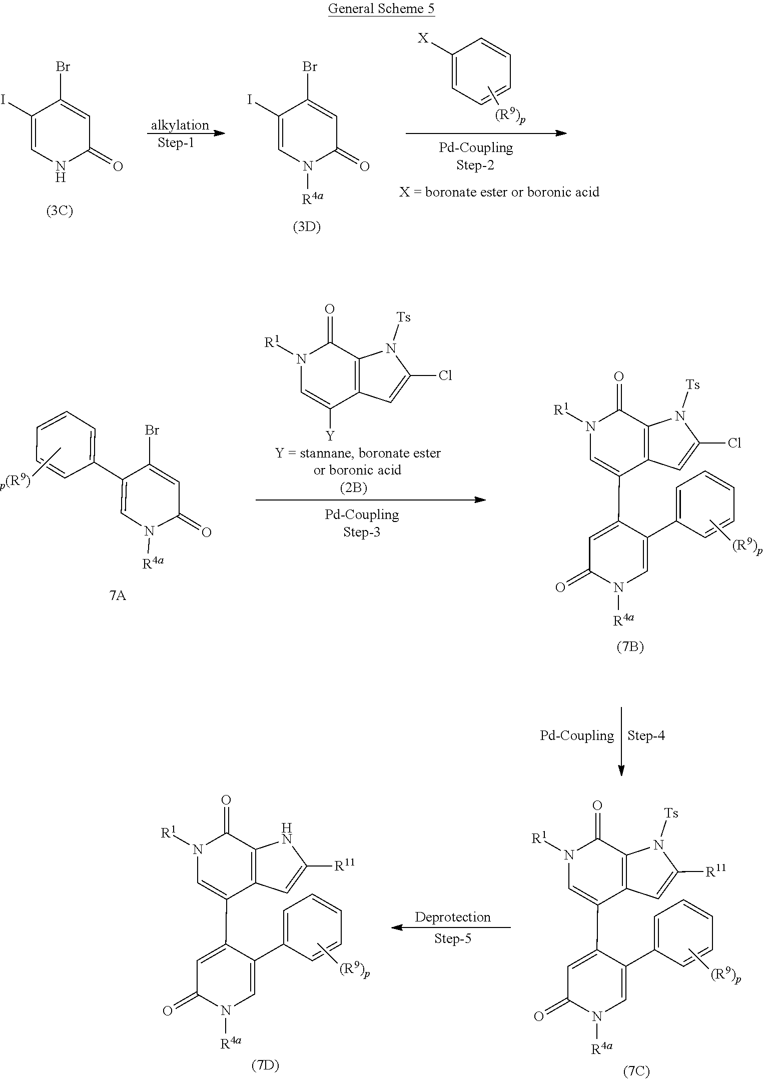

General Scheme 5 illustrates a general route for the preparation of compounds of the disclosure. From (3C) alkylation, Pd-coupling followed by Pd-coupling with (2B), gave (7B). Pd coupling and deprotection gave compounds of the disclosure such as (7D).

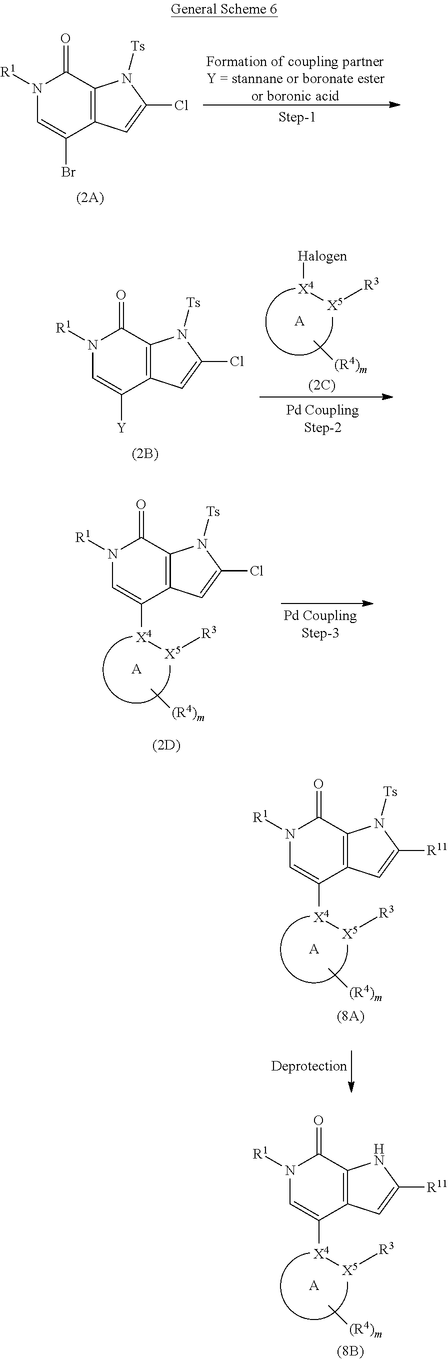

General Scheme 6 illustrates a general route for the preparation of compounds of the disclosure. From (2A) formation of stannane, boronate ester or boronic acid gave (2B), subsequent Pd-coupling with (2C), followed by Pd-coupling gave (8A) and deprotection gave compounds of the disclosure such as (8D).

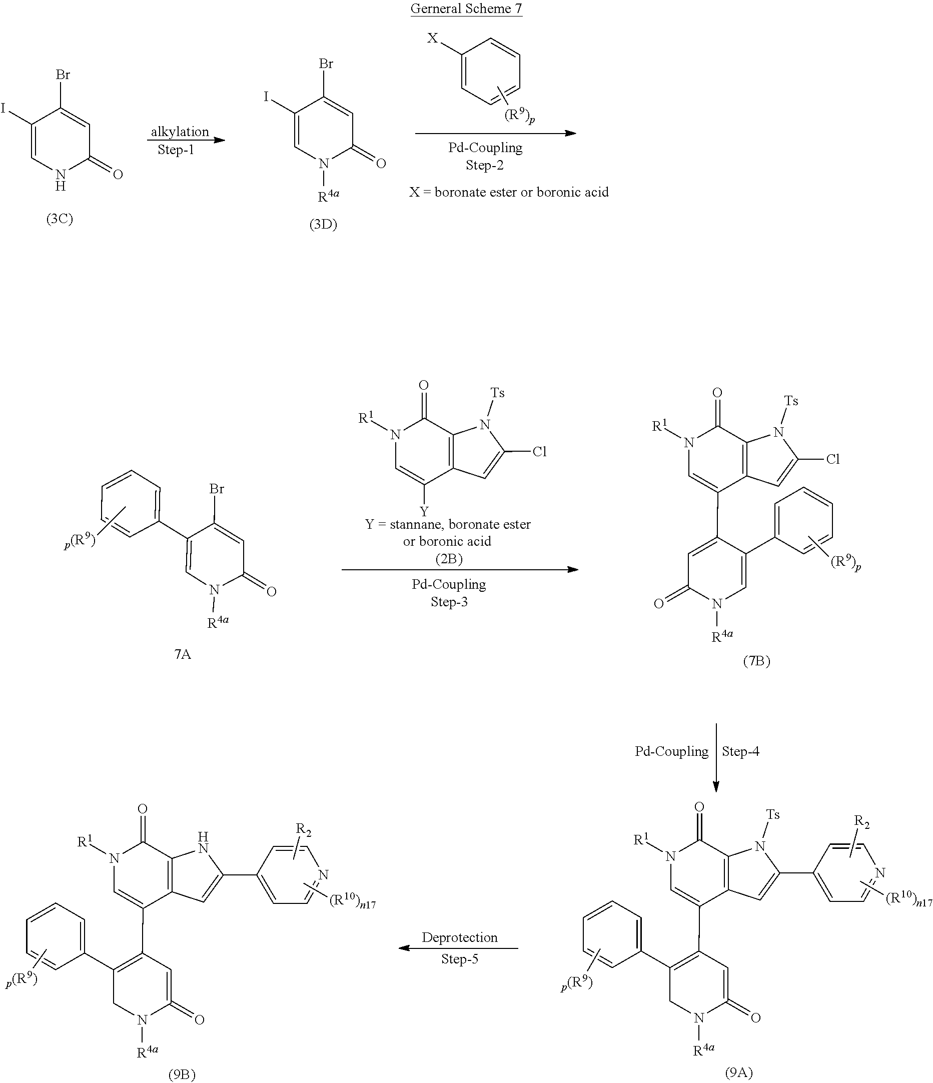

General Scheme 7 illustrates a general route for the preparation of compounds of the disclosure. From (3C) alkylation, Pd-coupling followed by Pd-coupling with (2B), gave (7B). Pd coupling and deprotection gave compounds of the disclosure such as (9B).

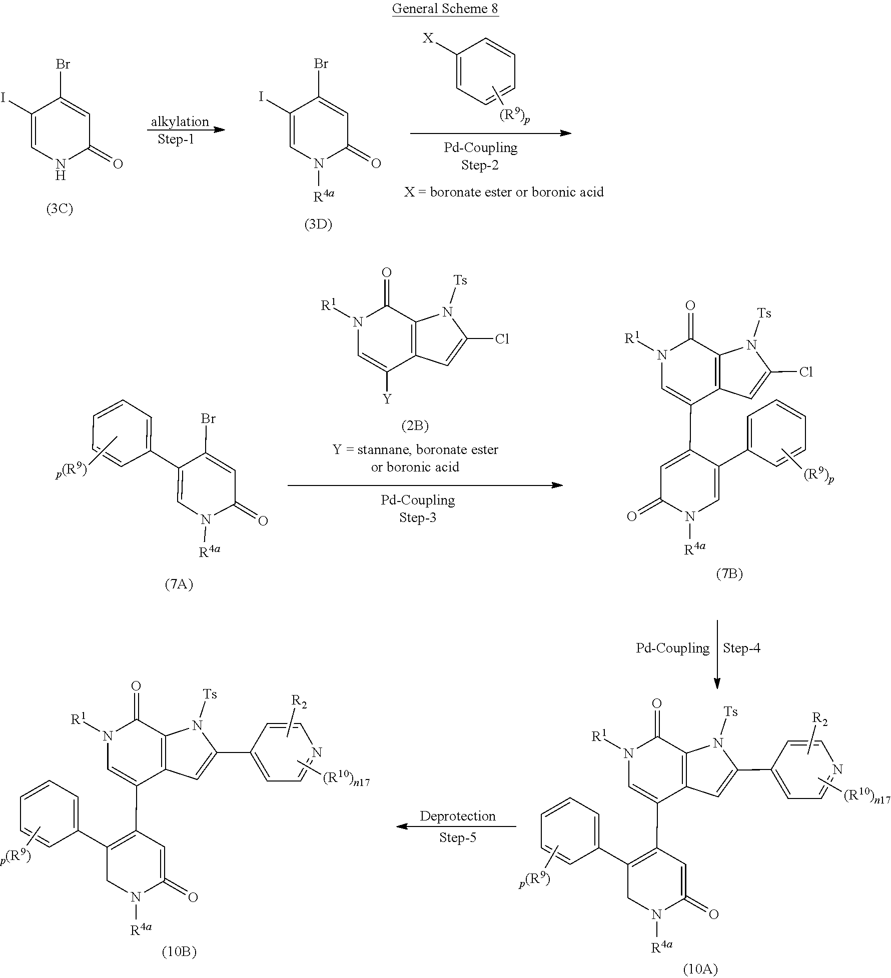

General Scheme 8 illustrates a general route for the preparation of compounds of the disclosure. From (3C) alkylation, Pd-coupling followed by Pd-coupling with (2B), gave (7B). Pd coupling and deprotection gave compounds of the disclosure such as (10B).

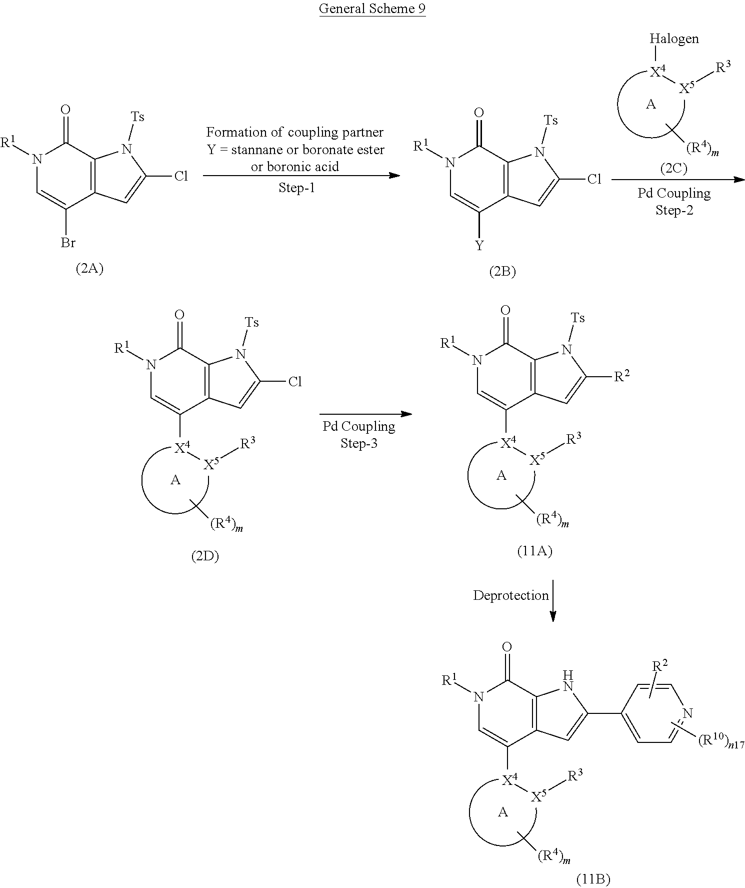

General Scheme 9 illustrates a general route for the preparation of compounds of the disclosure. From (2A) formation of stannane, boronate ester or boronic acid gave (2B), subsequent Pd-coupling with (2C), followed by Pd-coupling gave (11A) and deprotection gave compounds of the disclosure such as (11B).

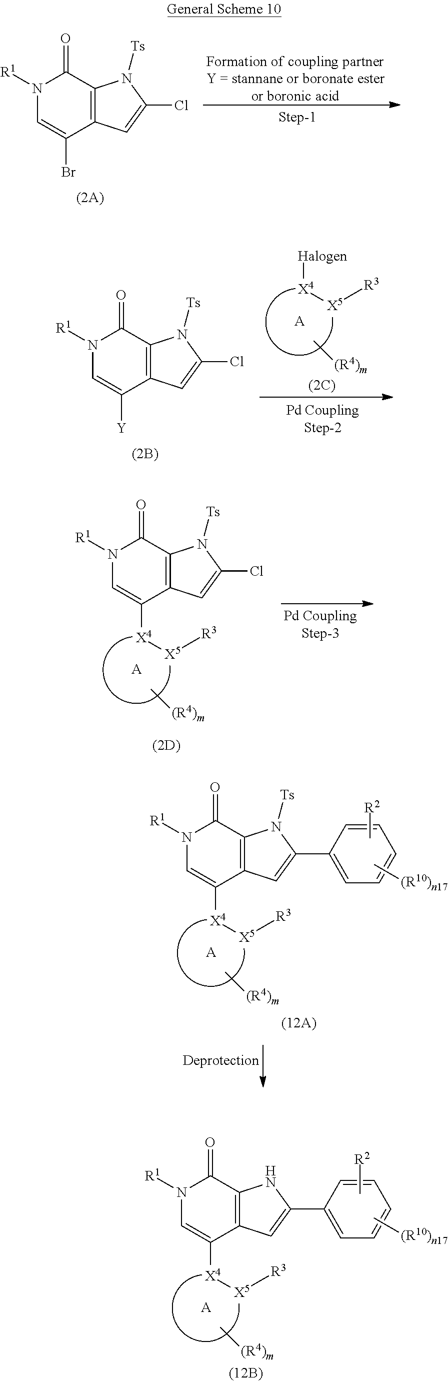

General Scheme 10 illustrates a general route for the preparation of compounds of the disclosure. From (2A) formation of stannane, boronate ester or boronic acid gave (21B), subsequent Pd-coupling with (2C), followed by Pd-coupling gave (12A) and deprotection gave compounds of the disclosure such as (12B).

Additional Schemes are illustrated below in Method F.

### Example 1: 6-methyl-2-(1-methyl-2-oxo-1,2-dihydropyridin-3-yl)-4-(1-methyl-2-oxo-5-phenyl-1,2-dihydropyridin-4-yl)-1,6-dihydro-7H-pyrrolo[2,3-c]pyridin-7-one

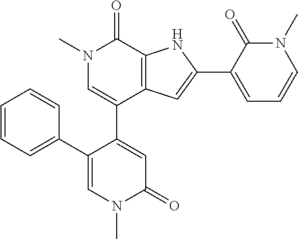 [SMILES: Cn1cc(-c2ccccc2)c(-c2cn(C)c(=O)c3[nH]c(-c4cccn(C)c4=O)cc23)cc1=O]

### Preparation 1: 2-chloro-4-(5,5-dimethyl-1,3,2-dioxaborinan-2-yl)-6-methyl-1-tosyl-1,6-dihydro-7H-pyrrolo[2,3-c]pyridin-7-one

To a stirred mixture of 4-bromo-2-chloro-6-methyl-1-tosyl-1,6-dihydro-7H-pyrrolo[2,3-c]pyridin-7-one (50.0 g, 120 mmol, 1.00 equiv) and 2-(5,5-dimethyl-1,3,2-dioxaborinan-2-yl)-5,5-dimethyl-1,3,2-dioxaborinane (163 g, 721 mmol, 6.00 equiv) in THF (150 mL) were added KOAc (23.6 g, 240 mmol, 2.00 equiv) and Pd(PPh₃)₂Cl₂ (8.44 g, 12.0 mmol, 0.100 equiv) at room temperature. The resulting mixture was stirred overnight at 60° C. under a nitrogen atmosphere. The reaction was quenched with water. The resulting mixture was extracted with EtOAc (3×100 mL). The combined organic layers were washed with brine, dried over anhydrous Na₂SO₄. After filtration, the filtrate was concentrated under reduced pressure. The residue was purified by silica gel column chromatography, eluted with PE/EA (1:4) to afford the product (70.0 g, crude) as a yellow solid. The residue was purified by trituration with MeOH (100 mL). The precipitated solids were collected by filtration and washed with MeOH (3×20 mL). The resulting solid was dried under reduced pressure. This resulted in the title compound as a light-yellow solid (26.0 g, 48.1%). LCMS: m/z=381 [M+H]⁺ of boronic acid. ¹H NMR (400 MHz, DMSO-d⁶) δ 8.11 (d, J=8.5 Hz, 2H), 7.77 (s, 1H), 7.54-7.42 (m, 2H), 6.92 (s, 1H), 3.75 (s, 4H), 3.46 (s, 3H), 2.41 (s, 3H), 0.95 (s, 6H).

### Preparation 2: 4-bromo-5-iodopyridin-2-amine

To a stirred mixture of 4-bromopyridin-2-amine (300 g, 1.73 mol, 1.00 equiv) in CHCl₃ (2.4 L) and DMF (0.7 L) were added NIS (780 g, 3.47 mol, 2.00 equiv) at room temperature. The resulting mixture was stirred overnight at 60° C. The resulting mixture was diluted with EtOAc (10.0 L). The resulting mixture was filtered. The filtrate was diluted with water. The resulting mixture was extracted with EtOAc (6×1.0 L). The combined organic layers were washed with Na₂S₂O₃, dried over anhydrous Na₂SO₄. After filtration, the filtrate was concentrated under reduced pressure. The crude product was purified by trituration with tert-Butyl methyl ether (0.5 L). After filtration, the filter cake was dried under vacuum to afford the title compound as a yellow green solid (260 g, 50%). LCMS: m/z=301 [M+H]⁺. ¹H NMR (300 MHz, DMSO-d⁶) δ 8.20 (s, 1H), 6.87 (s, 1H), 6.38 (br, 2H).

### Preparation 3: 4-bromo-5-iodopyridin-2(1H)-one

To a stirred mixture of 4-bromo-5-iodopyridin-2-amine (260 g, 0.869 mol, 1.00 equiv) in conc. H₂SO₄ (0.38 L, 7.20 mol, 8.28 equiv) and H₂O (1.54 L) were added NaNO2 (264 g, 3.83 mol, 4.40 equiv) in H₂O (0.480 L) dropwise at 0° C. The resulting mixture was stirred for 5 h at room temperature. The residue was neutralized to pH 7 with NH₃·H₂O. The resulting mixture was filtered, and the filter cake was washed with H₂O.

The filter cake was dried under reduced pressure. This resulted in the title compound as a yellow solid (280 g, crude). LCMS: m/z=302 [M+H]⁺. ¹H NMR (400 MHz, DMSO-d⁶) δ 11.89 (br, 1H), 7.87 (s, 1H), 6.91 (s, 1H).

### Preparation 4: 4-bromo-5-iodo-1-methylpyridin-2 (1H)-one

To a solution of 4-bromo-5-iodo-1H-pyridin-2-one (100 g, 0.333 mol, 1.00 equiv) in DMF (0.5 L) was added Cs₂CO₃ (217 g, 0.667 mol, 2.00 equiv). The mixture was stirred for 1 h at room temperature followed by the addition of CH₃I (71.0 g, 500 mmol, 1.50 equiv) dropwise. The resulting mixture was stirred for 5 h at room temperature. The precipitated solids were collected by filtration and washed with water. The collected solids were dried under vacuum. This resulted in the title compound as a brown solid (65.0 g, 62%). LCMS: m/z=316 [M+H]⁺. ¹H NMR (400 MHz, DMSO-d⁶) δ 8.23 (s, 1H), 6.92 (s, 1H), 3.38 (s, 3H).

### Preparation 5: 4-bromo-1-methyl-5-phenylpyridin-2 (1H)-one

To a stirred mixture of 4-bromo-5-iodo-1-methylpyridin-2 (1H)-one (70.0 g, 223 mmol, 1.00 equiv) and phenyl boronic acid (40.8 g, 334 mmol, 1.50 equiv) in DMF (250 mL) and H₂O (25 mL) were added Na₂CO₃ (47.3 g, 446 mmol, 2.00 equiv) and Pd(PPh₃)₄ (25.8 g, 22.3 mmol, 0.10 equiv) at room temperature. The resulting mixture was stirred at 100° C. under a nitrogen atmosphere for 6 h. The reaction was quenched with water (500 mL). The resulting mixture was extracted with EtOAc (3×500 mL). The combined organic layers were washed with brine, dried over anhydrous Na₂SO₄. After filtration, the filtrate was concentrated under reduced pressure. The residue was purified by silica gel column chromatography, eluted with PE/EA (3:1) to afford the crude product (30.0 g). The crude product was further purified by Prep-HPLC^{A} to afford the title compound as a white solid (23.0 g, 39%). LCMS: m/z=266 [M+H]⁺. ¹H NMR (400 MHz, CDCl₃) δ 7.41-7.33 (m, 3H), 7.33-7.26 (m, 2H), 7.23 (s, 1H), 6.95 (s, 1H), 3.50 (s, 3H).

### Preparation 6: 2-chloro-6-methyl-4-(1-methyl-2-oxo-5-phenyl-1,2-dihydropyridin-4-yl)-1-tosyl-1,6-dihydro-7H-pyrrolo[2,3-c]pyridin-7-one

To a mixture of 2-chloro-4-(5,5-dimethyl-1,3,2-dioxaborinan-2-yl)-6-methyl-1-tosyl-1,6-dihydro-7H-pyrrolo[2,3-c]pyridin-7-one (13.5 g, 30.0 mmol, 1.00 equiv) and 4-bromo-1-methyl-5-phenylpyridin-2 (1H)-one (8.74 g, 33.1 mmol, 1.10 equiv) in 1,4-dioxane (200 mL) and H₂O (40 mL) was added Na₂CO₃ (6.38 g, 60.2 mmol, 2.00 equiv) and Pd(PPh₃)₄ (3.48 g, 3.01 mmol, 0.100 equiv). The mixture was stirred at 60° C. under a nitrogen atmosphere overnight. The reaction was quenched with water. The resulting mixture was extracted with EtOAc (5×100 mL). The combined organic layers were washed with brine (3×100 mL), dried over anhydrous Na₂SO₄. After filtration, the filtrate was concentrated under reduced pressure. The residue was purified by silica gel column chromatography, eluted with PE/EA (1:3) to afford the title compound as a light-yellow solid (6.00 g, 38%). LCMS: m/z=520 [M+H]⁺.

### Preparation 7: 2-chloro-6-methyl-4-(1-methyl-2-oxo-5-phenyl-1,2-dihydropyridin-4-yl)-1,6-dihydro-7H-pyrrolo[2,3-c]pyridin-7-one

To a solution of 2-chloro-6-methyl-4-(1-methyl-2-oxo-5-phenyl-1,2-dihydropyridin-4-yl)-1-tosyl-1,6-dihydro-7H-pyrrolo[2,3-c]pyridin-7-one (6.00 g, 11.5 mmol, 1.00 equiv) in 1,4-dioxane (40 mL) and H₂O (8 mL) was added NaOH (4.62 g, 115 mmol, 10.0 equiv). The mixture was stirred for 1 h at 60° C. The resulting mixture was extracted with EtOAc (3×40 mL). The combined organic layers were washed with brine (2×20 mL), dried over anhydrous Na₂SO₄. After filtration, the filtrate was concentrated under reduced pressure. The residue was purified by silica gel column chromatography, eluted with EtOAc/MeOH (12:1) to afford the title compound as a light-yellow solid (4.00 g, 95%). LCMS: m/z=366 [M+H]⁺.

### Preparation 8: 6-methyl-2-(1-methyl-2-oxo-1,2-dihydropyridin-3-yl)-4-(1-methyl-2-oxo-5-phenyl-1,2-dihydropyridin-4-yl)-1,6-dihydro-7H-pyrrolo[2,3-c]pyridin-7-one

To a stirred mixture of 2-chloro-6-methyl-4-(1-methyl-2-oxo-5-phenyl-1,2-dihydropyridin-4-yl)-1,6-dihydro-7H-pyrrolo[2,3-c]pyridin-7-one (150 mg, 0.410 mmol, 1.00 equiv) and (1-methyl-2-oxo-1,2-dihydropyridin-3-yl)boronic acid (125 mg, 0.820 mmol, 2.00 equiv) in DME (4.0 mL) and H₂O (1.0 mL) at room temperature was added XPhos Pd G3 (34.7 mg, 0.0410 mmol, 0.100 equiv) and K₂CO₃ (113 mg, 0.820 mmol, 2.00 equiv). The resulting mixture at 80° C. under nitrogen atmosphere was stirred for 2 h. The resulting mixture was quenched with water. The resulting mixture was extracted with EtOAc (3×10 mL). The combined organic layers were dried over anhydrous Na₂SO₄. After filtration, the filtrate was concentrated under reduced pressure. The residue was purified by Prep-HPLC^{L} to afford the title compound as a white solid (51.5 mg, 28.6%). ¹H NMR (400 MHz, Methanol-d⁴) δ 8.01-7.91 (m, 1H), 7.81 (s, 1H), 7.73-7.65 (m, 1H), 7.24-7.11 (m, 5H), 7.03 (s, 1H), 6.74 (s, 1H), 6.52 (s, 1H), 6.50-6.41 (m, 1H), 3.71 (s, 3H), 3.65 (s, 3H), 3.54 (s, 3H). LCMS^{AI}: m/z 439 [M+H]⁺.

### Example 2: 6-methyl-4-(1-methyl-5-(3-((methylsulfonyl)methyl)phenyl)-2-oxo-1,2-dihydropyridin-4-yl)-2-(1-(trifluoromethyl)-1H-pyrazo-4-yl)-1,6-dihydro-7H-pyrrolo[2,3-c]pyridin-7-one

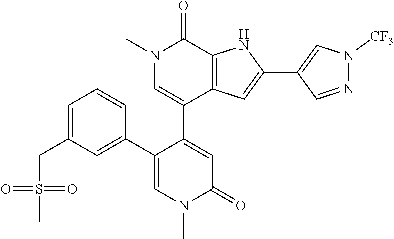 [SMILES: Cn1cc(-c2cccc(CS(C)(=O)=O)c2)c(-c2cn(C)c(=O)c3[nH]c(-c4cnn(C(F)(F)F)c4)cc23)cc1=O]

### Preparation 9: 1-bromo-3-((methylsulfonyl)methyl)benzene

To a solution of 1-bromo-3-(bromomethyl)benzene (10.0 g, 40.0 mmol, 1.00 equiv) in DMF (100.0 mL) was added sodium methanesulfonate (14.0 g, 118 mmol, 2.97 equiv) at room temperature under a nitrogen atmosphere. The resulting mixture was stirred for 2 h at 60° C. under a nitrogen atmosphere. The resulting mixture was concentrated under reduced pressure. The residue was diluted with water (1.0 L) and extracted with EtOAc (3×1.0 L). The combined organic layers were washed with brine (3×500 mL), dried over anhydrous Na₂SO₄. After filtration, the filtrate was concentrated under reduced pressure. The residue was purified by silica gel column chromatography, eluted with PE/EA (2:1) to afford the title compound as a white solid (6.20 g, 62%). ¹H NMR (400 MHz, Chloroform-d) δ 7.60-7.52 (m, 2H), 7.40-7.34 (m, 1H), 7.30 (t, J=7.8 Hz, 1H), 4.21 (s, 2H), 2.80 (s, 3H).

### Preparation 10: 4,4,5,5-tetramethyl-2-(3-((methylsulfonyl)methyl)phenyl)-1,3,2-dioxaborolane

To a stirred mixture of 1-bromo-3-((methylsulfonyl)methyl)benzene (6.00 g, 24.1 mmol, 1.00 equiv) and bis(pinacolato)diboron (24.4 g, 97.4 mmol, 4.00 equiv) in 1,4-dioxane (250 mL) was added KOAc (4.73 g, 48.2 mmol, 2.00 equiv) and Pd(dppf)Cl₂·CH₂Cl₂ (1.97 g, 2.41 mmol, 0.100 equiv) at room temperature. The resulting mixture was stirred overnight at 80° C. under a nitrogen atmosphere. The resulting mixture was concentrated under reduced pressure. The residue was diluted with water (300 mL). The resulting mixture was extracted with EtOAc (3×300 mL). The combined organic layers were washed with brine (2×300 mL), dried over anhydrous Na₂SO₄. After filtration, the filtrate was concentrated under reduced pressure. The residue was purified by silica gel column chromatography, eluted with PE/EA (10:1) to afford the crude product (11.0 g, brown liquid). The crude product was further purified by Prep-HPLC^{C} to afford the title compound as a white solid (2.40 g, 33%). LCMS: m/z=297 [M+H]⁺.

### Preparation 11: 4-bromo-1-methyl-5-(3-((methylsulfonyl)methyl)phenyl)pyridin-2 (1H)-one

To a stirred mixture of 4-bromo-5-iodo-1-methylpyridin-2 (1H)-one (1.50 g, 4.78 mmol, 1.00 equiv) and 4,4,5,5-tetramethyl-2-(3-((methylsulfonyl)methyl)phenyl)-1,3,2-dioxaborolane (1.62 g, 5.73 mmol, 1.20 equiv) in DMF (5.0 mL) and H₂O (0.5 mL) was added Pd(PPh₃)₄ (550 mg, 0.478 mmol, 0.100 equiv) and Na₂CO₃ (1.01 g, 9.56 mmol, 2.00 equiv) at room temperature under a nitrogen atmosphere. The resulting mixture was stirred for 2 h at 100° C. The resulting mixture was extracted with EtOAc (3×50 mL). The combined organic layers were washed with brine (2×50 mL), dried over anhydrous Na₂SO₄. After filtration, the filtrate was concentrated under reduced pressure. The residue was purified by silica gel column chromatography, eluted with MeOH/EA (1:5) to afford the title compound as a yellow solid (500 mg, 29%). LCMS: m/z=358 [M+H]⁺.

### Preparation 12: 2-chloro-6-methyl-4-(1-methyl-5-(3-((methylsulfonyl)methyl)phenyl)-2-oxo-1,2-dihydropyridin-4-yl)-1-tosyl-1,6-dihydro-7H-pyrrolo[2,3-c]pyridin-7-one

To a stirred mixture of 4-bromo-1-methyl-5-(3-((methylsulfonyl)methyl)phenyl)pyridin-2 (1H)-one (500 mg, 1.40 mmol, 1.10 equiv) and 2-chloro-4-(5,5-dimethyl-1,3,2-dioxaborinan-2-yl)-6-methyl-1-tosyl-1,6-dihydro-7H-pyrrolo[2,3-c]pyridin-7-one (572 mg, 1.28 mmol, 1.00 equiv) in DME (4.0 mL) and H₂O (1.0 mL) was added Na₂CO₃ (270 mg, 2.55 mmol, 2.00 equiv) and Pd(dppf)Cl₂·CH₂Cl₂ (104 mg, 0.128 mmol, 0.100 eq) at room temperature under a nitrogen atmosphere. The resulting mixture was stirred for 5 h at 60° C. The resulting mixture was extracted with EtOAc (3×50 mL). The combined organic layers were washed with brine (2×50 mL), dried over anhydrous Na₂SO₄. After filtration, the filtrate was concentrated under reduced pressure. The residue was purified by silica gel column chromatography, eluted with MeOH/EA (1:10) to afford the title compound as a yellow solid (390 mg, 50%). LCMS: m/z=612 [M+H]⁺.

### Preparation 13: 6-methyl-4-(1-methyl-5-(3-((methylsulfonyl)methyl)phenyl)-2-oxo-1,2-dihydropyridin-4-yl)-1-tosyl-2-(1-(trifluoromethyl)-1H-pyrazol-4-yl)-1,6-dihydro-7H-pyrrolo[2,3-c]pyridin-7-one

Following the procedure described in preparation 8, 2-chloro-6-methyl-4-(1-methyl-5-(3-((methylsulfonyl)methyl)phenyl)-2-oxo-1,2-dihydropyridin-4-yl)-1-tosyl-1,6-dihydro-7H-pyrrolo[2,3-c]pyridin-7-one (500 mg, 0.817 mmol, 1.00 equiv) and 4-(4,4,5,5-tetramethyl-1,3,2-dioxaborolan-2-yl)-1-(trifluoromethyl)-1H-pyrazole (321 mg, 1.23 mmol, 1.50 equiv) was reacted to give title compound as a yellow solid (300 mg, 51%). LCMS: m/z=712 [M+H]⁺.

### Preparation 14: 6-methyl-4-(1-methyl-5-(3-((methylsulfonyl)methyl)phenyl)-2-oxo-1,2-dihydropyridin-4-yl)-2-(1-(trifluoromethyl)-1H-pyrazol-4-yl)-1,6-dihydro-7H-pyrrolo[2,3-c]pyridin-7-one

Following the procedure described in preparation 7, 6-methyl-4-(1-methyl-5-(3-((methylsulfonyl)methyl)phenyl)-2-oxo-1,2-dihydropyridin-4-yl)-1-tosyl-2-(1-(trifluoromethyl)-1H-pyrazol-4-yl)-1,6-dihydro-7H-pyrrolo[2,3-c]pyridin-7-one (300 mg, 0.422 mmol, 1.00 equiv) was reacted to give title compound as orange solid (158.9 mg, 68%). LCMS^{B}: m/z=558 [M+H]⁺. ¹H NMR (400 MHz, DMSO-d⁶) δ 12.33-12.19 (m, 1H), 8.85 (s, 1H), 8.41 (t, J=0.9 Hz, 1H), 7.87 (s, 1H), 7.30-7.19 (m, 3H), 7.18-7.14 (m, 1H), 7.12 (s, 1H), 6.50 (s, 1H), 6.25 (d, J=2.1 Hz, 1H), 4.32 (s, 2H), 3.56 (s, 3H), 3.44 (s, 3H), 2.45 (s, 3H).

### Example 3: 4-(5-(3-(2-(dimethylamino)ethoxy)phenyl)-1-methyl-2-oxo-1,2-dihydropyridin-4-yl)-6-methyl-2-(1-(trifluoromethyl)-1H-pyrazol-4-yl)-1,6-dihydro-7H-pyrrolo[2,3-c]pyridin-7-one

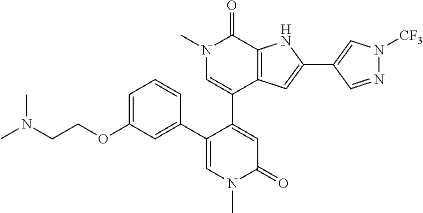 [SMILES: CN(C)CCOc1cccc(-c2cn(C)c(=O)cc2-c2cn(C)c(=O)c3[nH]c(-c4cnn(C(F)(F)F)c4)cc23)c1]

### Preparation 15: 2-(3-bromophenoxy)-N,N-dimethylethan-1-amine

To a stirred mixture of dimethylaminoethanol (5.00 g, 56.1 mmol, 1.00 equiv) and m-bromophenol (10.7 g, 61.7 mmol, 1.10 equiv) in THF (60 mL) were added PPh₃ (16.2 g, 61.7 mmol, 1.10 equiv) at room temperature under a nitrogen atmosphere. To the above mixture was added DIAD (12.5 g, 61.7 mmol, 1.10 equiv) dropwise over 10 min at 0° C. The resulting mixture was stirred for overnight at room temperature. The resulting mixture was extracted with EtOAc (3×30 mL), dried over anhydrous Na₂SO₄. After filtration, the filtrate was concentrated under reduced pressure. The crude product was purified by silica gel column chromatography, eluted with PE/EA (3:1) to afford the title compound as a white solid (2.0 g, 15%). LCMS: m/z=246 [M+H]⁺.

### Preparation 16: N,N-dimethyl-2-(3-(4,4,5,5-tetramethyl-1,3,2-dioxaborolan-2-yl)phenoxy)ethan-1-amine

To a mixture of [2-(3-bromophenoxy)ethyl]dimethylamine (4.00 g, 16.4 mmol, 1.00 equiv) and bis(pinacolato)diboron (16.6 g, 65.5 mmol, 4.00 equiv) in 1,4-dioxane (20 mL) was added Pd(dppf)Cl₂·CH₂Cl₂ (1.20 g, 1.64 mmol, 0.100 equiv) and AcOK (3.22 g, 32.8 mmol, 2.00 equiv) at room temperature. The resulting mixture was stirred for 2 h at 80° C. under a nitrogen atmosphere. The residue was concentrated under reduced pressure, and then purified by silica gel column chromatography, eluted with CH₂Cl₂/MeOH (1:1) to afford the title compound as a brown oil (520 mg, 11%). LCMS: m/z=292 [M+H]⁺.

### Preparation 17: 4-bromo-5-(3-(2-(dimethylamino)ethoxy)phenyl)-1-methylpyridin-2 (1H)-one

To a mixture of N,N-dimethyl-2-(3-(4,4,5,5-tetramethyl-1,3,2-dioxaborolan-2-yl)phenoxy)ethan-1-amine (500 mg, 1.72 mmol, 1.10 equiv) and 4-bromo-5-iodo-1-methylpyridin-2 (1H)-one (490 mg, 1.56 mmol, 1.00 equiv) in DMF (10 mL) and H₂O (1 mL) was added Pd(PPh₃)₄ (180 mg, 0.156 mmol, 0.100 equiv) and Na₂CO₃ (331 mg, 3.12 mmol, 2.00 equiv) at room temperature. The resulting mixture was stirred overnight at 100° C. under a nitrogen atmosphere. The reaction was quenched with water at room temperature. The aqueous layer was extracted with EtOAc (3×20 mL). The combined aqueous layers were concentrated under reduced pressure. The residue was purified by Prep-HPLC^{E} to afford the title compound as brown oil (185 mg, 34%). LCMS: m/z=353 [M+H]⁺.

### Preparation 18: 2-chloro-4-(5-(3-(2-(dimethylamino)ethoxy)phenyl)-1-methyl-2-oxo-1,2-dihydropyridin-4-yl)-6-methyl-1-tosyl-1,6-dihydro-7H-pyrrolo[2,3-c]pyridin-7-one

To a mixture of 4-bromo-5-(3-(2-(dimethylamino)ethoxy)phenyl)-1-methylpyridin-2 (1H)-one (177 mg, 0.504 mmol, 1.00 equiv) and 2-chloro-4-(5,5-dimethyl-1,3,2-dioxaborinan-2-yl)-6-methyl-1-tosyl-1,6-dihydro-7H-pyrrolo[2,3-c]pyridin-7-one (317 mg, 0.706 mmol, 1.40 equiv) in DME (10 mL) and H₂O (2 mL) was added Pd(dppf)Cl₂·CH₂Cl₂ (36.9 mg, 0.0500 mmol, 0.100 equiv) and Na₂CO₃ (107 mg, 1.01 mmol, 2.00 equiv) at room temperature. The resulting mixture was stirred overnight at 60° C. under a nitrogen atmosphere. The resulting mixture was concentrated under reduced pressure. The residue was purified by silica gel column chromatography, eluted with MeOH to afford the title compound as a yellow solid (152 mg, 50%). LCMS: m/z=607 [M+H]⁺.

### Preparation 19: 4-(5-(3-(2-(dimethylamino)ethoxy)phenyl)-1-methyl-2-oxo-1,2-dihydropyridin-4-yl)-6-methyl-1-tosyl-2-(1-(trifluoromethyl)-1H-pyrazol-4-yl)-1,6-dihydro-7H-pyrrolo[2,3-c]pyridin-7-one

Following the procedure described in preparation 8, 2-chloro-4-(5-(3-(2-(dimethylamino)ethoxy)phenyl)-1-methyl-2-oxo-1,2-dihydropyridin-4-yl)-6-methyl-1-tosyl-1,6-dihydro-7H-pyrrolo[2,3-c]pyridin-7-one (120 mg, 0.198 mmol, 1.00 equiv) and 4-(4,4,5,5-tetramethyl-1,3,2-dioxaborolan-2-yl)-1-(trifluoromethyl)pyrazole (103 mg, 0.396 mmol, 2.00 equiv) was reacted to give title compound (71.0 mg, 51%). LCMS: m/z=707 [M+H]⁺.

### Preparation 20: 4-(5-(3-(2-(dimethylamino)ethoxy)phenyl)-1-methyl-2-oxo-1,2-dihydropyridin-4-yl)-6-methyl-2-(1-(trifluoromethyl)-1H-pyrazol-4-yl)-1,6-dihydro-7H-pyrrolo[2,3-c]pyridin-7-one

Following the procedure described in preparation 7, 4-(5-(3-(2-(dimethylamino)ethoxy)phenyl)-1-methyl-2-oxo-1,2-dihydropyridin-4-yl)-6-methyl-1-tosyl-2-(1-(trifluoromethyl)-1H-pyrazol-4-yl)-1,6-dihydro-7H-pyrrolo[2,3-c]pyridin-7-one (71.0 mg, 0.100 mmol, 1.00 equiv) was reacted to give the crude product which was purified by Prep-HPLC^{G} to afford the title compound as white solid (9.60 mg, 17%). LCMS^{C}: m/z=553 [M+H]⁺. ¹H NMR (400 MHz, Methanol-d⁴) δ 8.51 (s, 1H), 8.13 (s, 1H), 7.82 (s, 1H), 7.18-7.05 (m, 2H), 6.85 (d, J=7.4 Hz, 1H), 6.80-6.72 (m, 2H), 6.71 (s, 1H), 6.31 (s, 1H), 3.88 (d, J=5.3 Hz, 2H), 3.70 (s, 3H), 3.55 (s, 3H), 2.65 (s, 2H), 2.27 (s, 6H).

### Example 4: 6-methyl-4-(1-methyl-2-oxo-5-(3-(piperazin-1-ylmethyl)phenyl)-1,2-dihydropyridin-4-yl)-2-(1-(trifluoromethyl)-1H-pyrazol-4-yl)-1,6-dihydro-7H-pyrrolo[2,3-c]pyridin-7-one

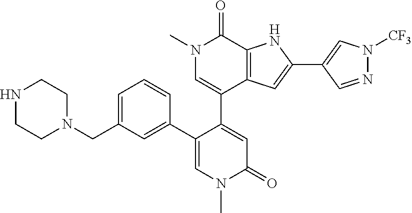 [SMILES: Cn1cc(-c2cccc(CN3CCNCC3)c2)c(-c2cn(C)c(=O)c3[nH]c(-c4cnn(C(F)(F)F)c4)cc23)cc1=O]

### Preparation 21: (3-((4-(tert-butoxycarbonyl)piperazin-1-yl)methyl)phenyl)boronic acid

To a solution of 3-(bromomethyl)phenylboronic acid (2.54 g, 11.8 mmol, 1.10 equiv) and tert-Butyl 1-piperazinecarboxylate (2.00 g, 10.7 mmol, 1.00 equiv) in MeCN (20 mL) was added K₂CO₃ (2.23 g, 16.1 mmol, 1.50 equiv). The reaction mixture was stirred for 3 h at 100° C. The resulting mixture was filtered. The filter cake was washed with acetonitrile (3×10 mL). The combined filtrate was concentrated under reduced pressure. The residue was purified by silica gel column chromatography, eluted with PE/EA (1:1) to afford the title compound as brown oil (2.90 g, crude). LCMS: m/z=321 [M+H]⁺

### Preparation 22: tert-butyl 4-(3-(4-bromo-1-methyl-6-oxo-1,6-dihydropyridin-3-yl)benzyl)piperazine-1-carboxylate

To a stirred mixture of 4-bromo-5-iodo-1-methylpyridin-2 (1H)-one (2.59 g, 8.25 mmol, 1.00 equiv) and (3-((4-(tert-butoxycarbonyl)piperazin-1-yl)methyl)phenyl)boronic acid (2.91 g, crude, 1.10 equiv) in DMF (30 mL) and H₂O (3.0 mL) were added Na₂CO₃ (1.75 g, 16.5 mmol, 2.00 equiv) and Pd(PPh₃)₄ (0.950 g, 0.825 mmol, 0.100 equiv) at room temperature. The resulting mixture was stirred for 2 h at 100° C. under a nitrogen atmosphere. The mixture was allowed to cool down to room temperature. The reaction was quenched with water. The resulting mixture was extracted with EtOAc (3×50 mL). The combined organic layers were washed with brine (2×50 mL), dried over anhydrous Na₂SO₄. After filtration, the filtrate was concentrated under reduced pressure. After filtration, the filtrate was concentrated under reduced pressure. The residue was purified by Prep-HPLCH to afford the title compound as a yellow solid (511 mg, 13%). LCMS: m/z=464 [M+H]⁺.

### Preparation 23: tert-butyl 4-(3-(4-(2-chloro-6-methyl-7-oxo-1-tosyl-6,7-dihydro-1H-pyrrolo[2,3-c]pyridin-4-yl)-1-methyl-6-oxo-1,6-dihydropyridin-3-yl)benzyl)piperazine-1-carboxylate

To a stirred mixture of tert-butyl 4-(3-(4-bromo-1-methyl-6-oxo-1,6-dihydropyridin-3-yl)benzyl)piperazine-1-carboxylate (500 mg, 1.10 mmol, 1.00 equiv) and 2-chloro-4-(5,5-dimethyl-1,3,2-dioxaborinan-2-yl)-6-methyl-1-tosyl-1,6-dihydro-7H-pyrrolo[2,3-c]pyridin-7-one (679 mg, 1.51 mmol, 1.40 equiv) in DME (10.0 mL) and H₂O (2.0 mL) were added Na₂CO₃ (229 mg, 2.16 mmol, 2.00 equiv) and Pd(dppf)Cl₂CH₂Cl₂ (89.8 mg, 0.110 mmol, 0.100 equiv) at room temperature. The resulting mixture was stirred for 5 h at 60° C. under a nitrogen atmosphere. The resulting mixture was extracted with EtOAc (3×15 mL). The combined organic layers were washed with brine, dried over anhydrous Na₂SO₄. After filtration, the filtrate was concentrated under reduced pressure. The residue was purified by silica gel column chromatography, eluted with EA/MeOH (10:1) to afford the title compound as a yellow solid (580 mg, 75%). LCMS: m/z=718 [M+H]⁺.

### Preparation 24: tert-butyl 4-(3-(1-methyl-4-(6-methyl-7-oxo-1-tosyl-2-(1-(trifluoromethyl)-1H-pyrazol-4-yl)-6,7-dihydro-1H-pyrrolo[2,3-c]pyridin-4-yl)-6-oxo-1,6-dihydropyridin-3-yl)benzyl)piperazine-1-carboxylate

Following the procedure described in preparation 8, tert-butyl 4-(3-(4-(2-chloro-6-methyl-7-oxo-1-tosyl-6,7-dihydro-1H-pyrrolo[2,3-c]pyridin-4-yl)-1-methyl-6-oxo-1,6-dihydropyridin-3-yl)benzyl)piperazine-1-carboxylate (500 mg, 0.696 mmol, 1.00 equiv) and 4-(4,4,5,5-Tetramethyl-1,3,2-dioxaborolan-2-yl)-1-(trifluoromethyl)-1H-pyrazole (219 mg, 0.835 mmol, was reacted to give title compound as a yellow solid (300 mg, 53%). LCMS: m/z=818 [M+H]⁺.

### Preparation 25: tert-butyl 4-(3-(1-methyl-4-(6-methyl-7-oxo-2-(1-(trifluoromethyl)-1H-pyrazol-4-yl)-6,7-dihydro-1H-pyrrolo[2,3-c]pyridin-4-yl)-6-oxo-1,6-dihydropyridin-3-yl)benzyl)piperazine-1-carboxylate

Following the procedure described in preparation 7, tert-butyl 4-(3-(1-methyl-4-(6-methyl-7-oxo-1-tosyl-2-(1-(trifluoromethyl)-1H-pyrazol-4-yl)-6,7-dihydro-1H-pyrrolo[2,3-c]pyridin-4-yl)-6-oxo-1,6-dihydropyridin-3-yl)benzyl)piperazine-1-carboxylate (290 mg, 0.355 mmol, 1.00 equiv) was reacted to give title compound (270 mg, crude). LCMS: m/z=664 [M+H]⁺.

### Preparation 26: 6-methyl-4-(1-methyl-2-oxo-5-(3-(piperazin-1-ylmethyl)phenyl)-1,2-dihydropyridin-4-yl)-2-(1-(trifluoromethyl)-1H-pyrazol-4-yl)-1,6-dihydro-7H-pyrrolo[2,3-c]pyridin-7-one

To a stirred solution of 4-(3-(1-methyl-4-(6-methyl-7-oxo-2-(1-(trifluoromethyl)-1H-pyrazol-4-yl)-6,7-dihydro-1H-pyrrolo[2,3-c]pyridin-4-yl)-6-oxo-1,6-dihydropyridin-3-yl)benzyl)piperazine-1-carboxylate (250 mg, 0.377 mmol, 1.00 equiv) in DCM (3 mL) was added TFA (1.0 mL) at room temperature. The resulting mixture was stirred for 4 h at room temperature. The resulting mixture was concentrated under reduced pressure. The residue was dissolved in MeOH (2.0 mL) and was filtered. The combined filtration was purified by Prep-HPLC^{I} to afford the title compound as a white solid (38.1 mg, 18%). LCMS^{D}: m/z=564 [M+H]⁺. ¹H NMR (400 MHz, Methanol-d⁶) δ 8.49 (s, 1H), 8.10 (apparent t, J=0.9 Hz, 1H), 7.80 (s, 1H), 7.40-7.23 (m, 2H), 7.14 (d, J=6.1 Hz, 2H), 7.05-6.94 (m, 1H), 6.70 (s, 1H), 6.23 (s, 1H), 3.70 (s, 3H), 3.57 (s, 3H), 3.27 (s, 2H), 2.67-2.65 (m, 4H), 2.03-1.93 (m, 4H).

### Example 5: 6-methyl-4-(1-(oxetan-3-yl)-2-oxo-5-phenyl-1,2-dihydropyridin-4-yl)-2-(1-(trifluoromethyl)-1H-pyrazol-4-yl)-1,6-dihydro-7H-pyrrolo[2,3-c]pyridin-7-one

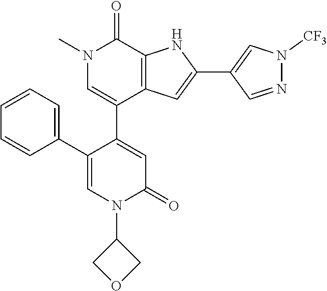 [SMILES: Cn1cc(-c2cc(=O)n(C3COC3)cc2-c2ccccc2)c2cc(-c3cnn(C(F)(F)F)c3)[nH]c2c1=O]

### Preparation 27: 4-bromo-5-iodo-1-(oxetan-3-yl)pyridin-2 (1H)-one

To a stirred mixture of 4-bromo-5-iodopyridin-2 (1H)-one (5.00 g, 16.6 mmol, 1.00 equiv) and K₂CO₃ (3.46 g, 25.0 mmol, 1.50 equiv) in DMF (80 mL) was added 3-iodooxetane (3.37 g, 18.3 mmol, 1.1 equiv) at room temperature. The resulting mixture was stirred for overnight at 100° C. The reaction was quenched with water. The resulting mixture was extracted with EtOAc (3×100 mL). The combined organic layers were washed with brine, dried over anhydrous Na₂SO₄. After filtration, the filtrate was concentrated under reduced pressure. The residue was purified by silica gel column chromatography, eluted with PE/EA (1:1) to afford the title compound as yellow solid (2.23 g, 38%). LCMS: m/z=358 [M+H]⁺.

### Preparation 28: 4-bromo-1-(oxetan-3-yl)-5-phenylpyridin-2 (1H)-one

To a stirred mixture of 4-bromo-5-iodo-1-(oxetan-3-yl)pyridin-2 (1H)-one (2.23 g, 6.28 mmol, 1.00 equiv) and phenylboronic acid (1.15 g, 9.42 mmol, 1.50 equiv) in DMF (20.0 mL) and H₂O (2.0 mL) was added Na₂CO₃ (1.33 g, 12.6 mmol, 2.00 equiv) and Pd(PPh₃)₄ (726 mg, 0.628 mmol, 0.10 equiv) at room temperature. The resulting mixture was stirred overnight at 100° C. under a nitrogen atmosphere. The resulting mixture was extracted with EtOAc (3×80 mL), dried over anhydrous Na₂SO₄. After filtration, the filtrate was concentrated under reduced pressure. The residue was purified by silica gel column chromatography, eluted with PE/EA (1:1) to afford the title compound as a yellow solid (900 mg, 46.8%). LCMS: m/z=308 [M+H]⁺.

### Preparation 29: 2-chloro-6-methyl-4-(1-(oxetan-3-yl)-2-oxo-5-phenyl-1,2-dihydropyridin-4-yl)-1-tosyl-1,6-dihydro-7H-pyrrolo[2,3-c]pyridin-7-one

To a stirred mixture of 4-bromo-1-(oxetan-3-yl)-5-phenylpyridin-2 (1H)-one (900 mg, 2.95 mmol, 1.00 equiv) and 2-chloro-4-(5,5-dimethyl-1,3,2-dioxaborinan-2-yl)-6-methyl-1-tosyl-1,6-dihydro-7H-pyrrolo[2,3-c]pyridin-7-one (1.98 g, 4.43 mmol, 1.50 equiv) in DME (10.0 mL) and H₂O (2.0 mL) was added Na₂CO₃ (625 mg, 5.90 mmol, 2.00 equiv) and Pd(dppf)Cl₂·CH₂Cl₂ (0.240 g, 0.295 mmol, 0.100 equiv) at room temperature. The resulting mixture was stirred overnight at 60° C. under a nitrogen atmosphere. The resulting mixture was extracted with EtOAc (3×50 mL), dried over anhydrous Na₂SO₄. After filtration, the filtrate was concentrated under reduced pressure. The residue was purified by silica gel column chromatography, eluted with MeOH/EA (10:1) to afford the title compound as a yellow solid (330 mg, 20%). LCMS: m/z=562 [M+H]⁺.

### Preparation 30: 6-methyl-4-(1-(oxetan-3-yl)-2-oxo-5-phenyl-1,2-dihydropyridin-4-yl)-1-tosyl-2-(1-(trifluoromethyl)-1H-pyrazol-4-yl)-1,6-dihydro-7H-pyrrolo[2,3-c]pyridin-7-one

Following the procedure described in preparation 8, 2-chloro-6-methyl-4-(1-(oxetan-3-yl)-2-oxo-5-phenyl-1,2-dihydropyridin-4-yl)-1-tosyl-1,6-dihydro-7H-pyrrolo[2,3-c]pyridin-7-one (310 mg, 0.553 mmol, 1.00 equiv) and 4-(4,4,5,5-tetramethyl-1,3,2-dioxaborolan-2-yl)-1-(trifluoromethyl)-1H-pyrazole (217 mg, 0.823 mmol, 1.50 equiv) was reacted to give title compound as yellow solid (78 mg, 21%). LCMS: m/z=662 [M+H]⁺.

### Preparation 31: 6-methyl-4-(1-(oxetan-3-yl)-2-oxo-5-phenyl-1,2-dihydropyridin-4-yl)-2-(1-(trifluoromethyl)-1H-pyrazol-4-yl)-1,6-dihydro-7H-pyrrolo[2,3-c]pyridin-7-one

Following the procedure described in preparation 7, 6-methyl-4-(1-(oxetan-3-yl)-2-oxo-5-phenyl-1,2-dihydropyridin-4-yl)-1-tosyl-2-(1-(trifluoromethyl)-1H-pyrazol-4-yl)-1,6-dihydro-7H-pyrrolo[2,3-c]pyridin-7-one (60 mg, 0.0907 mmol, 1.00 equiv) was reacted to give the crude product which was purified by Prep-HPLC^{J} to afford the title compound as a white solid (5.4 mg, 11.7%). LCMS^{E}: m/z=508 [M+H]⁺. ¹H NMR (400 MHz, DMSO-d⁶) δ 12.32 (s, 1H), 8.90 (s, 1H), 8.42 (s, 1H), 7.76 (s, 1H), 735-7.15 (m, 4H), 7.14 (d, J=6.9 Hz, 1H), 7.08 (d, 1H), 6.52 (s, 1H), 6.35 (s, 1H), 5.58-5.55 (m, 1H), 5.01-4.78 (m, 4H), 3.42 (s, 3H).

### Example 6: 2-(2,4-difluorophenyl)-4-(5-(1,3-dihydroisobenzofuran-4-yl)-1-methyl-2-oxo-1,2-dihydropyridin-4-yl)-6-methyl-1,6-dihydro-7H-pyrrolo[2,3-c]pyridin-7-one

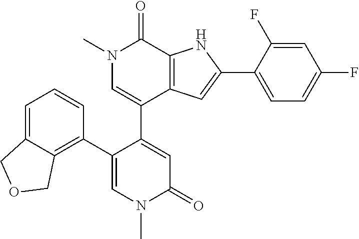 [SMILES: Cn1cc(-c2cccc3c2COC3)c(-c2cn(C)c(=O)c3[nH]c(-c4ccc(F)cc4F)cc23)cc1=O]

### Preparation 32: 4-(1-cyclopropyl-5-(2,6-dimethylphenoxy)-2-oxo-1,2-dihydropyridin-4-yl)-2-(2-fluoro-3-methoxyphenyl)-6-methyl-1,6-dihydro-7H-pyrrolo[2,3-c]pyridin-7-one

To a stirred mixture of 4-bromo-1,3-dihydro-2-benzofuran (950 mg, 4.77 mmol, 1.00 equiv) and bis(pinacolato) diboron (4.85 g, 19.0 mmol, 4.00 equiv) in dioxane (3.0 mL) at room temperature under nitrogen atmosphere were added KOAc (937 mg, 9.55 mmol, 2.00 equiv) and Pd(dppf)Cl₂·CH₂Cl₂ (389 mg, 0.477 mmol, 0.10 equiv). The resulting mixture was stirred at 80° C. under nitrogen atmosphere overnight. The resulting mixture was extracted with EtOAc (3×20 mL), dried over anhydrous Na₂SO₄. After filtration, the filtrate was concentrated under reduced pressure. The residue was purified by silica gel column chromatography, eluted with PE/EA (10:1) to afford the product. The product was purified by Prep-HPLC^{UV} to afford the title compound as a light-yellow liquid (750 mg, 63.8%). LCMS: m/z=247 [M+H]⁺

### Preparation 33: 4-bromo-5-(1,3-dihydroisobenzofuran-4-yl)-1-methylpyridin-2 (1H)-one

To a stirred solution of 4-bromo-5-iodo-1-methylpyridin-2-one (700 mg, 2.23 mmol, 1.00 equiv) and 2-(1,3-dihydro-2-benzofuran-4-yl)-4,4,5,5-tetramethyl-1,3,2-dioxaborolane (823 mg, 3.35 mmol, 1.50 equiv) in DMF (5.0 mL) and H₂O (0.5 mL) at room temperature under nitrogen atmosphere were added Na₂CO₃ (463 mg, 4.460 mmol, 2.0 equiv) and Pd(PPh₃)₄ (257 mg, 0.223 mmol, 0.100 equiv). The resulting mixture was stirred for additional 12 h at room temperature. The resulting mixture was extracted with EtOAc (3×30 mL), dried over anhydrous Na₂SO₄. After filtration, the filtrate was concentrated under reduced pressure. The crude product was purified by RP-HPLC^{BW} to afford the title compound as a brown liquid (400 mg, 58.5%). LCMS: m/z=308 [M+H]⁺

### Preparation 34: 4-[2-chloro-6-methyl-1-(4-methylbenzenesulfonyl)-7-oxopyrrolo[2,3-c]pyridin-4-yl]-5-(1,3-dihydro-2-benzofuran-4-yl)-1-methylpyridin-2-one

To a stirred solution of 4-bromo-5-(1,3-dihydro-2-benzofuran-4-yl)-1-methylpyridin-2-one (400 mg, 1.30 mmol, 1.00 equiv) and 2-chloro-6-methyl-1-(4-methylbenzenesulfonyl)-4-(4,4,5,5-tetramethyl-1,3,2-dioxaborolan-2-yl)pyrrolo[2,3-c]pyridin-7-one (786 mg, 1.70 mmol, 1.30 equiv) in DME (5.0 mL) and water (1.0 mL) at room temperature under nitrogen atmosphere were added Na₂CO₃ (280 mg, 2.61 mmol, 2.00 equiv) and Pd(dppf)Cl₂ (95.6 mg, 0.131 mmol, 0.10 equiv). The resulting mixture was stirred at 60° C. for additional 2 h. The resulting mixture was extracted with EtOAc (3×50 mL), dried over anhydrous Na₂SO₄. After filtration, the filtrate was concentrated under reduced pressure. The residue was purified by silica gel column chromatography, eluted with EA/MeOH (10:1) to afford the title compound as a yellow solid (400 mg, 54.5%). LCMS: m/z=562 [M+H]⁺

### Preparation 35: 4-[2-(2,4-difluorophenyl)-6-methyl-1-(4-methylbenzenesulfonyl)-7-oxopyrrolo[2,3-c]pyridin-4-yl]-5-(1,3-dihydro-2-benzofuran-4-yl)-1-methylpyridin-2-one

To a stirred solution of 4-[2-chloro-6-methyl-1-(4-methylbenzenesulfonyl)-7-oxopyrrolo[2,3-c]pyridin-4-yl]-5-(1,3-dihydro-2-benzofuran-4-yl)-1-methylpyridin-2-one (400 mg, 0.712 mmol, 1.00 equiv) and 2,4-difluorophenylboronic acid (169 mg, 1.07 mmol, 1.50 equiv) in DME (10.0 mL) and water (2.00 mL) at room temperature under nitrogen atmosphere were added Na₂CO₃ (198 mg, 1.42 mmol, 2.00 equiv) and Xphos Pd G3 (60.2 mg, 0.071 mmol, 0.10 equiv). The resulting mixture was stirred at 100° C. for additional 2 h. The resulting mixture was extracted with EtOAc (3×50 mL), dried over anhydrous Na₂SO₄. After filtration, the filtrate was concentrated under reduced pressure to afford the title compound as a black solid (500 mg, crude). The crude product was used in the next step directly without further purification. LCMS: m/z=640 [M+H]⁺

### Preparation 36: 4-[2-(2,4-difluorophenyl)-6-methyl-7-oxo-1H-pyrrolo[2,3-c]pyridin-4-yl]-5-(1,3-dihydro-2-benzofuran-4-yl)-1-methylpyridin-2-one

To a solution of 4-[2-(2,4-difluorophenyl)-6-methyl-1-(4-methylbenzenesulfonyl)-7-oxopyrrolo[2,3-c]pyridin-4-yl]-5-(1,3-dihydro-2-benzofuran-4-yl)-1-methylpyridin-2-one (500 mg, 0.782 mmol, 1.00 equiv) in MeOH (4.0 mL) and H₂O (1.0 mL) was added NaOH (312 mg, 7.82 mmol, 10.0 equiv). The mixture was stirred at 60° C. for 2 h. The resulting mixture was extracted with EtOAc (3×50 mL), dried over anhydrous Na₂SO₄. After filtration, the filtrate was concentrated under reduced pressure. The crude product (100 mg) was purified by Prep-HPLC^{BX} as a white solid (26.2 mg, 6.90%). ¹H NMR (400 MHz, CDCl₃) δ 10.49 (s, 1H), 7.78-7.72 (m, 1H), 7.34 (s, 1H), 7.24-7.20 (m, 1H), 7.13 (d, J=7.5 Hz, 1H), 7.08 (d, J=7.5 Hz, 1H), 6.98-6.92 (m, 2H), 6.88 (s, 1H), 6.64 (s, 1H), 6.52 (s, 1H), 5.00 (s, 2H), 4.70 (s, 2H), 3.66 (s, 3H), 3.39 (s, 3H). LCMS^{E}: m/z=486 [M+H]⁺

### Example 7: 4-(5-(3-(methoxymethyl)phenyl)-1-methyl-2-oxo-1,2-dihydropyridin-4-yl)-6-methyl-2-(1-(trifluoromethyl)-1H-pyrazol-4-yl)-1,6-dihydro-7H-pyrrolo[2,3-c]pyridin-7-one

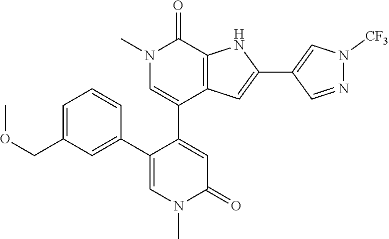 [SMILES: COCc1cccc(-c2cn(C)c(=O)cc2-c2cn(C)c(=O)c3[nH]c(-c4cnn(C(F)(F)F)c4)cc23)c1]

### Preparation 37: 2-(3-(methoxymethyl)phenyl)-4,4,5,5-tetramethyl-1,3,2-dioxaborolane

To a stirred mixture of 1-bromo-3-(methoxymethyl)benzene (4.96 g, 24.7 mmol, 1.00 equiv) and bis(pinacolato)diboron (12.5 g, 49.3 mmol, 2.00 equiv) in 1,4-dioxane (80 mL) were added KOAc (4.84 g, 49.3 mmol, 2.00 equiv) and Pd(dppf)Cl₂·CH₂Cl₂ (2.01 g, 2.47 mmol, 0.10 equiv) at room temperature. The resulting mixture was stirred overnight at 80° C. under a nitrogen atmosphere. The reaction was quenched with water. The resulting mixture was extracted with EtOAc (3×50 mL). The combined organic layers were washed with brine, dried over anhydrous Na₂SO₄. After filtration, the filtrate was concentrated under reduced pressure. The residue was purified by silica gel column chromatography, eluted with PE/EA (10:1) to afford the product (5.97 g, crude). The crude product was further purified by Prep-HPLC^{L} to afford the title compound as a yellow solid (2.92 g, 48%).

### Preparation 38: 4-bromo-5-(3-(methoxymethyl)phenyl)-1-methylpyridin-2 (1H)-one

To a stirred mixture of 4-bromo-5-iodo-1-methylpyridin-2 (1H)-one (2.91 g, 9.27 mmol, 1.00 equiv) and 4-bromo-5-(3-(methoxymethyl)phenyl)-1-methylpyridin-2 (1H)-one (3.45 g, 13.9 mmol, 1.50 equiv) in DMF (60 mL) and H₂O (12 mL) were added Na₂CO₃ (1.96 g, 18.5 mmol, 2.00 equiv) and Pd(PPh₃)₄ (1.07 g, 0.927 mmol, 0.100 equiv) at room temperature. The resulting mixture was stirred 2 h at 100° C. under a nitrogen atmosphere. The reaction was quenched with water at room temperature. The resulting mixture was extracted with EtOAc (3×80 mL). The combined organic layers were washed with brine, dried over anhydrous Na₂SO₄. After filtration, the filtrate was concentrated under reduced pressure. The residue was purified by silica gel column chromatography, eluted with PE/EA (1:1) to afford the product (1.32, crude). The crude product was further purified by Prep-HPLC^{M} to afford the title compound as a yellow liquid (564 mg, 19.7%). LCMS: m/z=310 [M+H]⁺.

### Preparation 39: 2-chloro-4-(5-(3-(mnethoxymethyl)phenyl)-1-methyl-2-oxo-1,2-dihydropyridin-4-yl)-6-methyl-1-tosyl-1,6-dihydro-7H-pyrrolo[2,3-c]pyridin-7-one

To a stirred mixture of 2-chloro-4-(5,5-dimethyl-1,3,2-dioxaborinan-2-yl)-6-methyl-1-tosyl-1,6-dihydro-7H-pyrrolo[2,3-c]pyridin-7-one (641 mg, 1.43 mmol, 1.00 equiv) and 4-bromo-5-(3-(methoxymethyl)phenyl)-1-methylpyridin-2 (1H)-one (550 mg, 1.79 mmol, 1.25 equiv) in DME (25 mL) and H₂O (5 mL) were added Na₂CO₃ (303 mg, 2.86 mmol, 2.00 equiv) and Pd(dppf)Cl₂·CH₂Cl₂ (116 mg, 0.143 mmol, 0.100 equiv) at room temperature. The resulting mixture was stirred overnight at 60° C. under a nitrogen atmosphere. The reaction was quenched with water. The resulting mixture was extracted with EtOAc (3×30 mL). The combined organic layers were washed with brine, dried over anhydrous Na₂SO₄. After filtration, the filtrate was concentrated under reduced pressure. The residue was purified by silica gel column chromatography, eluted with EtOAc/MeOH (7:1) to afford the title compound as yellow liquid (334 mg, 41%). LCMS: m/z=564 [M+H]⁺.

### Preparation 40: 4-(5-(3-(methoxymethyl)phenyl)-1-methyl-2-oxo-1,2-dihydropyridin-4-yl)-6-methyl-1-tosyl-2-(1-(trifluoromethyl)-1H-pyrazol-4-yl)-1,6-dihydro-7H-pyrrolo[2,3-c]pyridin-7-one

Following the procedure described in preparation 8, 2-chloro-4-(5-(3-(methoxymethyl)phenyl)-1-methyl-2-oxo-1,2-dihydropyridin-4-yl)-6-methyl-1-tosyl-1,6-dihydro-7H-pyrrolo[2,3-c]pyridin-7-one (320 mg, 0.567 mmol, 1.00 equiv) and 4-(4,4,5,5-tetramethyl-1,3,2-dioxaborolan-2-yl)-1-(trifluoromethyl)-1H-pyrazole (178 mg, 0.680 mmol, 1.20 equiv) was reacted to give title compound as yellow liquid (175 mg, 46%). LCMS: m/z=664 [M+H]⁺.

### Preparation 41: 4-(5-(3-(methoxymethyl)phenyl)-1-methyl-2-oxo-1,2-dihydropyridin-4-yl)-6-methyl-2-(1-(trifluoromethyl)-1H-pyrazol-4-yl)-1,6-dihydro-7H-pyrrolo[2,3-c]pyridin-7-one

Following the procedure described in preparation 7, 4-(5-(3-(methoxymethyl)phenyl)-1-methyl-2-oxo-1,2-dihydropyridin-4-yl)-6-methyl-1-tosyl-2-(1-(trifluoromethyl)-1H-pyrazol-4-yl)-1,6-dihydro-7H-pyrrolo[2,3-c]pyridin-7-one (158 mg, 0.238 mmol, 1.00 equiv) was reacted to give the crude product which was purified by Prep-HPLC^{N} to afford the title compound as a white solid (39.2 mg, 32%). LCMS^{N}: m/z=510 [M+H]⁺. ¹H NMR (400 MHz, Methanol-d⁴) δ 8.49 (s, 1H), 8.12 (s, 1H), 7.81 (s, 1H), 7.25-7.19 (m, 1H), 7.19-7.12 (m, 2H), 7.11-7.06 (m, 2H), 6.71 (s, 1H), 6.23 (s, 1H), 4.29 (s, 2H), 3.70 (s, 3H), 3.56 (s, 3H), 3.09 (s, 3H).

### Example 8: 4-(5-(4-(methoxymethyl)phenyl)-1-methyl-2-oxo-1,2-dihydropyridin-4-yl)-6-methyl-2-(1-(trifluoromethyl)-1H-pyrazol-4-yl)-1,6-dihydro-7H-pyrrolo[2,3-c]pyridin-7-one

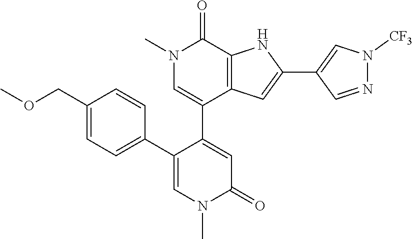 [SMILES: COCc1ccc(-c2cn(C)c(=O)cc2-c2cn(C)c(=O)c3[nH]c(-c4cnn(C(F)(F)F)c4)cc23)cc1]

### Preparation 42: 2-(4-(methoxymethyl)phenyl)-4,4,5,5-tetramethyl-1,3,2-dioxaborolane

To a mixture of 1-bromo-4-(methoxymethyl) benzene (3.00 g, 14.9 mmol, 1.00 equiv) and bis(pinacolato)diboron (7.58 g, 29.8 mmol, 2.00 equiv) in 1,4-dioxane (40 mL) was added Pd(dppf)Cl₂·CH₂Cl₂ (1.09 g, 1.49 mmol, 0.100 equiv) and KOAc (2.93 g, 29.8 mmol, 2.00 equiv) at room temperature under a nitrogen atmosphere. The resulting mixture was stirred overnight at 80° C. The reaction was quenched with water. The resulting mixture was extracted with EtOAc (5×50 mL). The combined organic layers were washed with brine (3×50 mL), dried over anhydrous Na₂SO₄. After filtration, the filtrate was concentrated under reduced pressure. The residue was purified by silica gel column chromatography, eluted with EA/MeOH (10:1) to afford the crude product (2.30 g, crude) and further purified by Prep-HPLC^{O} to afford the title compound as a yellow oil (1.56 g, 42%).

### Preparation 43: 4-bromo-5-(4-(methoxymethyl)phenyl)-1-methylpyridin-2 (1H)-one

To a mixture of 2-[4-(methoxymethyl)phenyl]-4,4,5,5-tetramethyl-1,3,2-dioxaborolane (1.50 g, 6.04 mmol, 1.50 equiv) and 4-bromo-5-iodo-1-methylpyridin-2 (1H)-one (1.27 g, 4.03 mmol, 1.00 equiv) in DMF (30.0 mL) and H₂O (3.0 mL) was added Pd(PPh₃)₄ (0.470 g, 0.403 mmol, 0.100 equiv) and Na₂CO₃ (0.850 g, 8.06 mmol, 2.00 equiv) at room temperature under a nitrogen atmosphere. The resulting mixture was stirred for 1 h at 100° C. The reaction was quenched with water. The resulting mixture was extracted with EtOAc (5×80 mL). The combined organic layers were washed with brine (3×80 mL), dried over anhydrous Na₂SO₄. After filtration, the filtrate was concentrated under reduced pressure. The residue was purified by Prep-HPLC^{c} to afford the title compound as yellow oil (440 mg, 35%). LCMS: m/z=310 [M+H]⁺.

### Preparation 44: 2-chloro-4-(5-(4-(methoxymethyl)phenyl)-1-methyl-2-oxo-1,2-dihydropyridin-4-yl)-6-methyl-1-tosyl-1,6-dihydro-7H-pyrrolo[2,3-c]pyridin-7-one

To a mixture of 4-bromo-5-(4-(methoxymethyl)phenyl)-1-methylpyridin-2 (1H)-one (400 mg, 1.30 mmol, 1.00 equiv) and 2-chloro-4-(5,5-dimethyl-1,3,2-dioxaborinan-2-yl)-6-methyl-1-(4-methylbenzenesulfonyl)pyrrolo[2,3-c]pyridin-7-one (757 mg, 1.69 mmol, 1.30 equiv) in DME (10.0 mL) and H₂O (2.0 mL) was added Pd(dppf)Cl₂·CH₂Cl₂ (105 mg, 0.130 mmol, 0.100 equiv) and Na₂CO₃ (275 mg, 2.60 mmol, 2.00 equiv) at room temperature. The resulting mixture was stirred overnight at 60° C. under a nitrogen atmosphere. The reaction was quenched with water. The resulting mixture was extracted with EtOAc (5×50 mL). The combined organic layers were washed with brine (3×10 mL), dried over anhydrous Na₂SO₄. After filtration, the filtrate was concentrated under reduced pressure. The residue was purified by silica gel column chromatography, eluted with EA/MeOH (10:1) to afford the title compound as a yellow solid (426 mg, 58%). LCMS: m/z=564 [M+H]⁺.

### Preparation 45: 4-(5-(4-(methoxymethyl)phenyl)-1-methyl-2-oxo-1,2-dihydropyridin-4-yl)-6-methyl-1-tosyl-2-(1-(trifluoromethyl)-1H-pyrazol-4-yl)-1,6-dihydro-7H-pyrrolo[2,3-c]pyridin-7-one

Following the procedure described in preparation 8, 2-chloro-4-(5-(4-(methoxymethyl)phenyl)-1-methyl-2-oxo-1,2-dihydropyridin-4-yl)-6-methyl-1-tosyl-1,6-dihydro-7H-pyrrolo[2,3-c]pyridin-7-one (400 mg, 0.727 mmol, 1.00 equiv) and 4-(4,4,5,5-tetramethyl-1,3,2-dioxaborolan-2-yl)-1-(trifluoromethyl)-1H-pyrazole (229 mg, 0.872 mmol, 1.20 equiv) was reacted to give title compound as a yellow solid (340 mg, 70%). LCMS: m/z=664 [M+H]⁺.

### Preparation 46: 4-(5-(4-(methoxymethyl)phenyl)-1-methyl-2-oxo-1,2-dihydropyridin-4-yl)-6-methyl-2-(1-(trifluoromethyl)-1H-pyrazol-4-yl)-1,6-dihydro-7H-pyrrolo[2,3-c]pyridin-7-one

Following the procedure described in preparation 7, 4-(5-(4-(methoxymethyl)phenyl)-1-methyl-2-oxo-1,2-dihydropyridin-4-yl)-6-methyl-1-tosyl-2-(1-(trifluoromethyl)-1H-pyrazol-4-yl)-1,6-dihydro-7H-pyrrolo[2,3-c]pyridin-7-one (320 mg, 0.482 mmol, 1.00 equiv) was reacted to give the crude product which was purified by Prep-HPLC^{Q} to afford the title compound as white solid (93.3 mg, 37%). LCMS^{H}: m/z=510 [M+H]⁺. ¹H NMR (400 MHz, Methanol-d⁴) δ 8.50 (s, 1H), 8.12 (s, 1H), 7.81 (s, 1H), 7.20-7.17 (m, 4H), 7.06 (s, 1H), 6.72 (s, 1H), 6.26 (s, 1H), 4.33 (s, 2H), 3.70 (s, 3H), 3.55 (s, 3H), 3.19 (s, 3H).

### Example 9: 4-(5-(4-(2-methoxyethoxy)phenyl)-1-methyl-2-oxo-1,2-dihydropyridin-4-yl)-6-methyl-2-(1-(trifluoromethyl)-1H-pyrazol-4-yl)-1,6-dihydro-7H-pyrrolo[2,3-c]pyridin-7-one

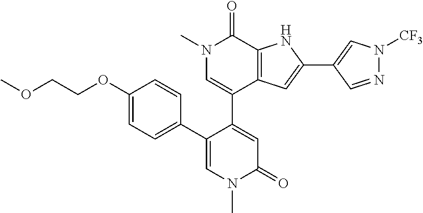 [SMILES: COCCOc1ccc(-c2cn(C)c(=O)cc2-c2cn(C)c(=O)c3[nH]c(-c4cnn(C(F)(F)F)c4)cc23)cc1]

### Preparation 47: 1-bromo-3-(2-methoxyethoxy)benzene

To a stirred mixture of m-bromophenol (20.0 g, 116 mmol, 1.00 equiv) and 2-bromoethyl methyl ether (36.2 g, 262 mmol, 2.25 equiv) in DMF (200 mL) were added K₂CO₃ (24.0 g, 174 mmol, 1.50 equiv) and KI (2.00 g, 11.6 mmol, 0.100 equiv) at room temperature. The resulting mixture was stirred for 24 h at 70° C. The reaction was quenched with water at room temperature. The resulting mixture was extracted with EtOAc (2×100 mL). The combined organic layers were washed with brine (2×100 mL), dried over anhydrous Na₂SO₄. After filtration, the filtrate was concentrated under reduced pressure. The residue was purified by Prep-HPLC^{R} to afford the title compound as a white solid (20.0 g, 60%). ¹H NMR (400 MHz, CDCl₃) δ 7.15-7.07 (m, 3H), 6.88-6.85 (m, 1H), 4.10-4.08 (m, 2H), 3.75-3.72 (m, 2H), 3.44 (s, 3H).

### Preparation 48: 2-(3-(2-methoxyethoxy)phenyl)-4,4,5,5-tetramethyl-1,3,2-dioxaborolane

To a stirred mixture of 1-bromo-3-(2-methoxyethoxy)benzene (20.0 g, 87.0 mmol, 1.00 equiv) and bis(pinacolato)diboron (88.4 g, 348 mmol, 4.00 equiv) in dioxane (250 mL) was added Pd(dppf)Cl₂·CH₂Cl₂ (7.10 g, 8.7 mmol, 0.100 equiv) and KOAc (17.1 g, 174 mmol, 2.00 equiv) at room temperature. The resulting mixture was stirred overnight at 80° C. under a nitrogen atmosphere. The reaction was quenched with water at room temperature. The resulting mixture was extracted with EtOAc (3×80 mL). The combined organic layers were washed with brine (2×80 mL), dried over anhydrous Na₂SO₄. After filtration, the filtrate was concentrated under reduced pressure. The residue was purified by Prep-HPLCs to afford the title compound as yellow oil (25.0 g, crude).

### Preparation 49: 4-bromo-5-(3-(2-methoxyethoxy)phenyl)-1-methylpyridin-2 (1H)-one

To a stirred mixture of 2-(3-(2-methoxyethoxy)phenyl)-4,4,5,5-tetramethyl-1,3,2-dioxaborolane (10.0 g, crude) and 4-bromo-5-iodo-1-methylpyridin-2 (1H)-one (3.00 g, 9.58 mmol, 1.00 equiv) in DMF (100 mL) and H₂O (10 mL) were added Na₂CO₃ (2.03 g, 19.2 mmol, 2.00 equiv) and Pd(PPh₃)₄ (1-11 g, 0.960 mmol, 0.100 equiv) at room temperature. The resulting mixture was stirred for 4 h at 100° C. under a nitrogen atmosphere. The reaction was quenched with water at room temperature. The resulting mixture was extracted with EtOAc (4×60 mL). The combined organic layers were washed with brine (2×60 mL), dried over anhydrous Na₂SO₄. After filtration, the filtrate was concentrated under reduced pressure. The residue was purified by Prep-HPLC^{T} to afford the title compound as yellow solid (1.10 g, 34%). LCMS: m/z=340 [M+H]⁺.

### Preparation 50: 2-chloro-4-(5-(3-(2-methoxyethoxy)phenyl)-1-methyl-2-oxo-1,2-dihydropyridin-4-yl)-6-methyl-1-tosyl-1,6-dihydro-7H-pyrrolo[2,3-c]pyridin-7-one

To a stirred mixture of 4-bromo-5-(3-(2-methoxyethoxy)phenyl)-1-methylpyridin-2 (1H)-one (1.14 g, 3.38 mmol, 1.00 equiv) and 2-chloro-4-(5,5-dimethyl-1,3,2-dioxaborinan-2-yl)-6-methyl-1-tosyl-1,6-dihydro-7H-pyrrolo[2,3-c]pyridin-7-one (2.27 g, 5.07 mmol, 1.50 equiv) in DME (5 mL) and H₂O (1 mL) were added Pd(dppf)Cl₂·CH₂Cl₂ (0.276 g, 0.338 mmol, 0.100 equiv) and Na₂CO₃ (0.717 g, 6.76 mmol, 2.00 equiv) at room temperature. The resulting mixture was stirred overnight at 60° C. under a nitrogen atmosphere. The reaction was quenched with water at room temperature. The resulting mixture was extracted with EtOAc (4×10 mL). The combined organic layers were washed with brine (2×10 mL), dried over anhydrous Na₂SO₄. After filtration, the filtrate was concentrated under reduced pressure. The residue was purified by silica gel column chromatography, eluted with EA/MeOH (10:1) to afford the title compound as light-yellow solid (1.50 g, 80% purity, 60%). LCMS: m/z=594 [M+H]⁺.

### Preparation 51: 4-(5-(3-(2-methoxyethoxy)phenyl)-1-methyl-2-oxo-1,2-dihydropyridin-4-yl)-6-methyl-1-tosyl-2-(1-(trifluoromethyl)-1H-pyrazol-4-yl)-1,6-dihydro-7H-pyrrolo[2,3-c]pyridin-7-one

Following the procedure described in preparation 8, 2-chloro-4-(5-(3-(2-methoxyethoxy)phenyl)-1-methyl-2-oxo-1,2-dihydropyridin-4-yl)-6-methyl-1-tosyl-1,6-dihydro-7H-pyrrolo[2,3-c]pyridin-7-one (600 mg, 1.01 mmol, 1.00 equiv) and 4-(4,4,5,5-tetramethyl-1,3,2-dioxaborolan-2-yl)-1-(trifluoromethyl)-1H-pyrazole (529 mg, 2.02 mmol, 2.00 equiv) was reacted to give title compound as a yellow solid (490 mg, 70%). LCMS: m/z=694 [M+H]⁺.

### Preparation 52: 4-(5-(3-(2-methoxyethoxy)phenyl)-1-methyl-2-oxo-1,2-dihydropyridin-4-yl)-6-methyl-2-(1-(trifluoromethyl)-1H-pyrazol-4-yl)-1,6-dihydro-7H-pyrrolo[2,3-c]pyridin-7-one

Following the procedure described in preparation 7, 4-(5-(3-(2-methoxyethoxy)phenyl)-1-methyl-2-oxo-1,2-dihydropyridin-4-yl)-6-methyl-1-tosyl-2-(1-(trifluoromethyl)-1H-pyrazol-4-yl)-1,6-dihydro-7H-pyrrolo[2,3-c]pyridin-7-one (480 mg, 0.692 mmol, 1.00 equiv) was reacted to give the crude product which was purified by Prep-HPLC^{U} to afford the title compound as white solid (164.9 mg, 44%). ¹H NMR (400 MHz, DMSO-d⁶) δ 12.32 (s, 1H), 8.90 (s, 1H), 8.44 (s, 1H), 7.91 (s, 1H), 7.13 (s, 1H), 7.07-7.03 (m, 1H), 6.82 (s, 1H), 6.72-6.65 (m, 2H), 6.48 (s, 1H), 6.38 (s, 1H), 3.93-3.91 (m, 2H), 3.55 (s, 3H), 3.49-3.46 (m, 5H), 3.28 (s, 3H). LCMS^{C}: m/z=540 [M+H]⁺.

### Example 10: 3-chloro-6-methyl-4-(1-methyl-2-oxo-5-phenyl-1,2-dihydropyridin-4-yl)-2-(1-(trifluoromethyl)-1H-pyrazol-4-yl)-1,6-dihydro-7H-pyrrolo[2,3-c]pyridin-7-one

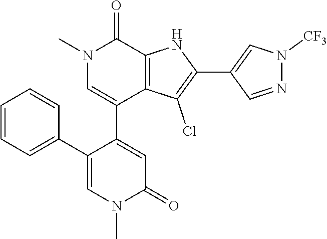 [SMILES: Cn1cc(-c2ccccc2)c(-c2cn(C)c(=O)c3[nH]c(-c4cnn(C(F)(F)F)c4)c(Cl)c23)cc1=O]

### Preparation 53: 3-chloro-6-methyl-4-(1-methyl-2-oxo-5-phenyl-1,2-dihydropyridin-4-yl)-2-(1-(trifluoromethyl)-1H-pyrazol-4-yl)-1,6-dihydro-7H-pyrrolo[2,3-c]pyridin-7-one

To a solution of 6-methyl-4-(1-methyl-2-oxo-5-phenyl-1,2-dihydropyridin-4-yl)-2-(1-(trifluoromethyl)-1H-pyrazol-4-yl)-1,6-dihydro-7H-pyrrolo[2,3-c]pyridin-7-one (200 mg, 0.430 mmol, 1.00 equiv) in DMF (8.0 mL) at 0° C. was added NCS (57.4 mg, 0.430 mmol, 1.00 equiv). The resulting mixture was stirred for 4 h at room temperature. The reaction was quenched with water at room temperature. The resulting mixture was extracted with EtOAc (4×10 mL). The combined organic layers were washed with brine (2×10 mL), dried over anhydrous Na₂SO₄. After filtration, the filtrate was concentrated under reduced pressure. The crude product was purified by Prep-HPLC^{V} to afford the title compound as a white solid (136.7 mg, 64%). ¹H NMR (400 MHz, DMSO-d⁶) δ 12.75 (s, 1H), 8.88 (s, 1H), 8.51 (s, 1H), 7.80 (s, 1H), 7.24 (s, 1H), 7.18-7.09 (m, 5H), 6.43 (s, 1H), 3.56 (s, 3H), 3.51 (s, 3H). LCMS^{J}: m/z=500 [M+H]⁺.

### Example 11: 4-(5-(2,6-dimethylphenoxy)-1-methyl-2-oxo-1,2-dihydropyridin-4-yl)-6-methyl-2-(1-(trifluoromethyl)-1H-pyrazol-4-yl)-1,6-dihydro-7H-pyrrolo[2,3-c]pyridin-7-one

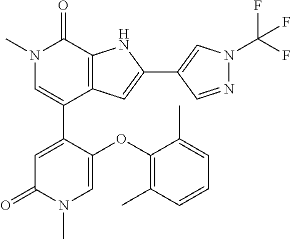 [SMILES: Cc1cccc(C)c1Oc1cn(C)c(=O)cc1-c1cn(C)c(=O)c2[nH]c(-c3cnn(C(F)(F)F)c3)cc12]

### Preparation 54: (E)-2-(5-bromo-2-methoxy-3-nitropyridin-4-yl)-N, N-dimethylethen-1-amine

5-bromo-2-methoxy-4-methyl-3-nitropyridine (1000 g, 4048.5 mmol) was dissolved in DMF (10000 mL) at room temperature (in 20 L 4N RBF with overhead stirrer). LiOMe (15.37 g, 404.8 mmol) was added to the reaction mixture at room temperature. The suspension was allowed to stir at 95° C. for 30 min. DMF-DMA (3860 g, 32388 mmol) was added drop wise over a period of 2 h. The resulting dark solution was heated at 95° C. for 16 h. The reaction mixture was cooled to 0° C. and water (10000 mL) was slowly added over 2 h. The resulting suspension was stirred for 1 h at 0° C. and filtered. The collected residue red solid was washed with water (1000 mL×3) and dried overnight under vacuum at 45° C. for 16 h in vacuum oven to afford (E)-2-(5-bromo-2-methoxy-3-nitropyridin-4-yl)-N, N-dimethylethen-1-amine (925 g, 75%). The material was directly used in preparation 2 without further purification. ¹H NMR: (400 MHz, DMSO-d⁶) δ 824 (s, 1H), 7.04 (d, J=13.6 Hz, 1H), 4.80 (d, J=13.6 Hz, 1H), 3.87 (s, 3H), 2.90 (s, 6H).

### Preparation 55: 4-bromo-7-methoxy-1H-pyrrolo[2,3-c]pyridine

(E)-2-(5-bromo-2-methoxy-3-nitropyridin-4-yl)-N, N-dimethylethen-1-amine (250 g, 830.5 mmol) was partially dissolved in mixture of ethanol (2500 mL) and glacial acetic acid (2500 mL) (in 10 L 4N RBF with overhead stirrer) followed by portion wise addition of Iron powder (232.5 g, 4152.8 mmol) over 10-15 min. The reaction mixture was refluxed at 90° C. for 2 h. The resulting solution was cooled to 50° C. and concentrated under reduced pressure (2-3 h, 70% solvent reduction). The resulting reaction mixtures were cooled to room temperature and diluted with DCM (3000 mL). The resulting reaction mixture was stirred at room temperature for 1 h. The resulting reaction mixture was filtered through a pad of celite and washed with DCM (3000 mL). The combined filtrate was partitioned with water (5000 mL) which again stirred at room temperature for 30 min. Organic layer was separated and washed with brine solution (5700 mL). The resulting organic layer was concentrated under reduced pressure. The resulting green residue was diluted with hexane (1000 mL) at room temperature and stirred for 3-4 h. The resulting precipitates were collected by filtration and washed with hexane (300 mL×2) and obtained solid material was dried under vacuum at 45° C. for 16 h yielding 4-bromo-7-methoxy-1H-pyrrolo[2,3-c]pyridine (175 g, 87%) as off-white solid. ¹H NMR (400 MHz, DMSO-d⁶) δ 12.17 (s, 1H), 7.75 (s, 1H), 7.55 (t, J=2.4 Hz, 1H), 6.42 (t, J=2.4 Hz, 1H), 4.00 (s, 3H). LCMS: m/z=229 [M+H]⁺

### Preparation 56: 4-bromo-7-methoxy-1-tosyl-1H-pyrrolo[2,3-c]pyridine

Sodium hydride (60% w/w, 132.74 g, 3318.5 mmol) was suspended in DMF (5000 mL) under nitrogen at 0° C. in 20 L RBF with overhead stirrer. 4-bromo-7-methoxy-1H-pyrrolo[2,3-c]pyridine (500 g, 2212 mmol) was dissolved in DMF (5000 mL) and drop wise added over a period of 1.5 h (evolution of gas was observed). The reaction mixture was stirred at 0° C. for 1 h. A solution of 4-Methylbenzenesulfonyl chloride (632.6 g, 3318.5 mmol) in DMF (5000 mL) was drop wise added to the reaction mixture at 0° C. over a period of 2 h. The resulting grey suspension gradually warmed to room temperature and stirred at room temperature for 16 h. The reaction mixture was cooled at 0° C. and slowly added water (15000 mL) over a period of 2 h. The resulting reaction was stirred at 0° C. for 1 h. The resulting precipitates were collected by filtration and washed with ice cold water (1000 mL×5) followed by hexane (1000 mL×5). The obtained solid material was dried under vacuum at 45° C. for 16 h yielding 4-bromo-7-methoxy-1-tosyl-1H-pyrrolo[2,3-c]pyridine (760 g, 92%) as off white solid. ¹H NMR (400 MHz, DMSO-d⁶) δ 8.17 (d, J=3.6 Hz, 1H), 7.99 (s, 1H), 7.85 (d, J=8.4 Hz, 2H), 7.45 (d, J=8.2 Hz, 2H), 6.80 (d, J=4.0 Hz, 1H), 3.81 (s, 3H), 2.49 (s, 3H). LCMS: m/z=381 [M+H]⁺.

### Preparation 57: 4-bromo-2-chloro-7-methoxy-1-tosyl-1H-pyrrolo[2, 3-c]pyridine

4-bromo-7-methoxy-1-tosyl-1H-pyrrolo[2, 3-c]pyridine (200 g, 526.3 mmol) was dissolved in dry THF (4000 mL, 20 V) at room temperature. LDA (1 M in THF) (684 mL, 684.2 mmol) was dropwise added to the reaction mixture at −78° C. (mixture of dry ice and EtOAc) for 30 min. The resulting reaction mixture was allowed to stir at −78° C. for 2 h. Hexachloroethane (199.3 g, 842 mmol) in dry THF (1000 mL) was dropwise added to the reaction mixture at −78° C. for 15 min. The resulting reaction mixture was allowed to stir at −78° C. to rt for 4 h. The reaction mixture was quenched in NH₄Cl solution (3000 mL) aqueous was extracted with ethyl acetate (3×2000 mL). The organic fraction was dried over Na₂SO₄, filtered and evaporated to yield 4-bromo-2-chloro-7-methoxy-1-tosyl-1H-pyrrolo[2,3-c]pyridine (160 g, 68%) as a brown solid. ¹H NMR (400 MHz, DMSO-d⁶) δ 8.08 (s, 1H), 7.95 (d, J=8.4, 2H), 7.53 (d, J=8.0 Hz, 2H), 7.05 (s, 1H), 3.87 (s, 3H), 2.42 (s, 3H). LCMS: m/z=417 [M+1H]⁺

### Preparation 58: 4-bromo-2-chloro-1-tosyl-1, 6-dihydro-7H-pyrrolo[2, 3-c]pyridin-7-one

4-bromo-2-chloro-7-methoxy-1-tosyl-1H-pyrrolo[2,3-c]pyridine (320 g, 772.9 mmol) was dissolved in acetonitrile (3200 mL, 10 V) at room temperature. Sodium iodide (173.8 g, 1159.4 mmol) was added to the reaction mixture and allowed stir at room temperature for 15 min. The resulting solution was cooled at 0° C. and dropwise added Trimethylsilyl chloride (147.2 mL, 1159.4 mmol). The resulting mixture was allowed to stir at ambient temperature for 1 h. Water (160 mL, 0.5 V) was added to the reaction mixture and heated at 65° C. for 3 h. The resulting mixture was quenched with ice-cold water (3200 mL) and stirred for 30 min. The resulting residue was filtered and triturated by n-hexane (320 mL) and diethyl ether (320 mL). The solid was dried overnight under vacuum at 45° C. to give white solid (295 g, 95%). ¹H NMR (400 MHz, DMSO-d⁶) δ 11.65 (s, 1H), 8.11 (d, J=8.4 Hz, 2H), 7.49-7.42 (m, 3H), 6.78 (s, 1H), 2.40 (s, 3H). LCMS: m/z=403 [M+H]⁺

### Preparation 59: 4-bromo-2-chloro-6-methyl-1-tosyl-1, 6-dihydro-7H-pyrrolo[2, 3-c]pyridin-7-one

4-bromo-2-chloro-1-tosyl-1,6-dihydro-7H-pyrrolo[2,3-c]pyridin-7-one (295 g, 737.6 mmol) was dissolved in DMF (5900 mL, 20 V) at room temperature. Potassium carbonate (203 g, 1475 mmol) was portion wise added to the reaction mixture at 0° C. and the resulting solution was allowed to stir at same temperature for 30 min. Methyl iodide (69.2 mL, 1106.4 mmol) was dropwise added to the reaction mixture at 0° C. and allows to stir at room temperature for 4 h. The reaction mixture was quenched with ice cold water (5900 mL) to afford a yellow coloured precipitate. The resulting precipitate was filtered and washed with water (3000 mL) and hexane (3000 mL). The solid was dried overnight under vacuum at 45° C. to give a white solid (270 g, 88%). ¹H NMR (400 MHz, DMSO-d⁶) δ 8.13 (d, J=8.4 Hz, 2H), 7.90 (s, 1H), 7.50 (d, J=8.0 Hz, 2H), 6.81 (s, 1H), 3.43 (s, 3H), 2.41 (s, 3H). LCMS: m/z=417 [M+H]⁺

### Preparation 60: 2-chloro-6-methyl-1-tosyl-4-(tributylstannyl)-1,6-dihydro-7H-pyrrolo[2,3-c]pyridin-7-one

1,4 dioxane (50 mL, 10 V) was degassed under argon for 30 min. 4-bromo-2-chloro-6-methyl-1-tosyl-1,6-dihydro-7H-pyrrolo[2,3-c]pyridin-7-one (5.0 g, 1.20 mmol) and Bis(tributyltin) (11.6 mL, 22.9 mmol) was added to the reaction mixture followed by Tetrakis (1.4 g, 1.20 mmol) at room temperature. The resulting mixture was allowed to stir at 130° C. for 3 h. The resulting solution was filtered through celite-pad and filtrate was diluted with water (50 mL) and ethyl acetate (50 mL). The combined organics were dried over Na₂SO₄, filtered and evaporated. The resulting residue was purified by normal phase chromatography, eluting with (30:70) ethyl acetate/hexane to yield 2-chloro-6-methyl-1-tosyl-4-(tributylstannyl)-1,6-dihydro-7H-pyrrolo[2,3-c]pyridin-7-one (2.5 g, 28%) as an off-white solid. ¹H NMR: (400 MHz, DMSO-d⁶) δ 8.14 (d, J=8.0 Hz, 2H), 7.48 (d, J=8.0 Hz, 2H), 7.22 (s, 1H), 6.58 (s, 1H), 3.45 (s, 3H), 2.40 (s, 3H), 1.50-1.43 (m, 6H), 1.32-1.22 (m, 6H), 1.11-1.06 (m, 6H), 0.87 (t, J=8.0 Hz, 9H).

### Preparation 61: 2-chloro-5-fluoropyridine 1-oxide

Trifluoroacetic acid (2.4 L, 8 V) was charged in 5 L four neck RBF at to 0° C. 2-chloro-5-fluoropyridine (300 g, 229.02 mmol) was dropwise added to the pre-cooled mixture by using additional funnel over 20 min. 30% hydrogen peroxide (450 mL, 39.70 mmol) was slowly added to reaction mixture. The resulting mixture was heated at 75° C. for 16 h. Trifluoroacetic acid (2.3 L) was separated by vacuum distillation. The resulting mixture was diluted by using cold water (2000 mL) and 70% of aqueous ammonia solution (500 mL). The aqueous fraction was extracted with dichloromethane (6×2000 mL). The combined organics were washed (brine), dried (Na₂SO₄), filtered and evaporated. The residue was triturated by n-pentane. Solvent reduction to give light brown solid (314 g, 93%). ¹H NMR (400 MHz, DMSO-d⁶) δ 8.82 (m, 1H), 7.88 (m, 1H), 7.44 (m, 1H). LCMS: m/z=148 [M+H]⁺

### Preparation 62: 2-chloro-5-fluoro-4-nitropyridine 1-oxide

2-chloro-5-fluoropyridine 1-oxide (100 g, 677.9 mmol) was dissolved in H₂SO₄ (500 mL, 5 V) in 5 L four neck RBF at room temperature. The resulting mixture was heated at 90° C. The pre-stirred solution of H₂SO₄ (1000 mL, 10 V) and HNO₃ (283 mL, 6777.9 mmol) at 0° C. was dropwise added to the reaction mixture at 90° C. The reaction mixture was allowed to stir at same temperature for 2 h. The resulting mixture was cooled at room temperature and ice (5 kg) was portion wise added under stirring. The aqueous fraction was extracted with ethyl acetate (2×1000 mL), The combined organics were washed (NaHCO₃ solution), dried (Na₂SO₄), filtered and evaporated. The resulting mixture was triturated by hexane (2×50 mL). Solvent reduction to give light-yellow solid (68 g, 53%). ¹H NMR (400 MHz, DMSO-d⁶) δ 8.82 (d, J=6.8 Hz, 1H), 8.82 (d, J=8.8 Hz, 1H). LCMS: m/z=193 [M+H]⁺.

### Preparation 63: 2-chloro-5-(2,6-dimethylphenoxy)-4-nitropyridine 1-oxide

2-chloro-5-fluoro-4-nitropyridine 1-oxide (178.5 g, 934.5 mmol) was dissolved in dimethylformamide (892.5 mL, 5 V) under nitrogen. Potassium carbonate (768.2 g, 5,607 mmol) was added to the reaction mixture and allowed to stir for 30 min. 2,6-Dimethylphenol (119.71 g, 981.28 mmol) was added to the reaction mixture and stirred for 4 h at room temperature. The resulting mixture was quenched with water (1000 mL) and stirred for 30 min. The resulting suspension was filtered and triturated by n-hexane. The solid was dried overnight under vacuum at 45° C. (245 g, 89%). ¹H NMR (400 MHz, DMSO-d⁶) δ 8.72 (s, 1H), 7.42 (s, 1H), 7.23 (m, 3H), 2.14 (s, 6H). LCMS: m/z=296 [M+H]⁺.

### Preparation 64: 2, 4-dibromo-5-(2,6-dimethylphenoxy) pyridine 1-oxide

2-chloro-5-(2,6-dimethylphenoxy)-4-nitropyridine 1-oxide (40 g, 13.5 mmol) was dissolved in acetyl bromide (200 mL, 5 V) at room temperature under nitrogen. The resulting mixture was heated at 75° C. and acetyl bromide (200 mL, 5 V) was added dropwise. The reaction mixture was stirred at same temperature for 4 h. The resulting mixture was slowly poured onto cold water (5000 mL). The aqueous fraction was extracted with ethyl acetate (2×2000 mL). The combined organics were washed (NaHCO₃ solution), dried (Na₂SO₄), filtered and evaporated. Solvent reduction to give light brown solid (26 g, 52%). ¹H NMR (400 MHz, DMSO-d⁶) d 8.42 (s, 1H), 7.21 (m, 4H), 2.10 (s, 6H). LCMS: m/z=374 [M+1H]⁺

### Preparation 65: 2,4-dibromo-5-(2,6-dimethylphenoxy) pyridine

4-dibromo-5-(2,6-dimethylphenoxy) pyridine 1-oxide (200 g, 539.08 mmol) was dissolved in chloroform (2000 mL, 10 V) under nitrogen. Phosphorus tribromide (200 mL, 1 V) was dropwise added to reaction mixture over 30 min and allowed to stir at 55° C. for 2 h. The resulting mixture was slowly poured into cold water (5000 mL). The aqueous fraction was extracted with ethyl acetate (2×2000 mL). The combined organics were washed (NaHCO₃ solution), dried (Na₂SO₄), filtered and evaporated. Solvent reduction to give light brown solid (130 g, 67%). ¹H NMR (400 MHz, DMSO-d⁶) δ 8.16 (s, 1H), 7.36 (s, 1H), 7.20-7.18 (m, 3H), 2.07 (s, 6H). LCMS: m7/z=358 [M+H]⁺.

### Preparation 66: 4-bromo-5-(2,6-dimethyl phenoxy) pyridin-2-ol

2,4-dibromo-5-(2,6-dimethylphenoxy) pyridine (10 g, 28.24 mmol) was dissolved in Tert-butanol (125 mL, 12.5 V) at room temperature in autoclave. potassium hydroxide (15.8 g, 282.40 mmol) was added to the reaction mixture and allowed to stir at 150° C. for 16 h. The reaction mixture was cooled to 0° C. and ice was added. The mixture was acidified by 2 N HCl to adjust pH 2. The resulting fraction was extracted with DCM (2×700 mL). The combined organics were washed (brine solution), dried (Na₂SO₄), filtered and evaporated. The residue was triturated by ethyl acetate (100 mL). Solvent reduction gives off-white solid Solvent reduction to give light brown solid (4 g, 48%) which was used directly without further purification. ¹H NMR (400 MHz, DMSO-d⁶) δ 11.27 (br s, 1H), 7.18-7.09 (m, 3H), 6.92 (s, 1H), 6.36 (s, 1H), 2.09 (s, 6H). LCMS: m/z=294 [M+H]⁺.

### Preparation 67: 4-bromo-5-(2,6-dimethylphenoxy)-1-methylpyridin-2 (1H)-one

4-bromo-5-(2,6-dimethylphenoxy) pyridin-2-ol (22.78 g, 77.7 mmol) was dissolved in DMF (227.8 mL, 10 V) under nitrogen. Cesium carbonate (76.0 g, 233.2 mmol) was added to the reaction mixture and cooled at 0° C. Methyl iodide (176.53 g, 1243.8 mmol) was drop wise added to the reaction mixture and allowed to stir at room temperature for 2 h. The reaction mixture was quenched with water (500 mL) and extracted by DCM (3×500 mL). The combined organics were washed (brine solution), dried (Na₂SO₄), filtered and evaporated. The residue was purified on silica, eluting with 39.0:61.0 acetonitrile:H₂O. Solvent reduction to give off white solid (10 g, 42%). ¹H NMR (400 MHz, DMSO-d⁶) δ 7.17-7.08 (m, 3H), 6.92 (s, 1H), 6.73 (s, 1H), 3.26 (s, 3H), 2.12 (s, 6H). LCMS: m/z=308 [M+H]⁺.

### Preparation 68: 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c]pyridin-7-one

2-chloro-6-methyl-1-tosyl-4-(tributylstannyl)-1, 6-dihydro-7H-pyrrolo[2,3-c]pyridin-7-one (2.6 g, 4.15 mmol) and 4-bromo-5-(2,6-dimethylphenoxy)-1-methylpyridin-2 (1H)-one (1.27 g, 4.15 mmol) was dissolved in Toluene (24 mL, 12 V). Reaction mixture was purged under argon for 30 min. Tetrakis (0.47 g, 0.41 mmol) was added to the reaction mixture and resulting mixture was allowed to stir at 120° C. for 3 h. The reaction mixture was diluted with water (50 mL) and extracted by ethyl acetate (50 mL). The combined organics were dried over Na₂SO₄, filtered and evaporated. The residue was purified by reverse phase chromatography, eluting with (60:40) acetonitrile/water. Lyophilized to give an off-white solid. (0.95 g, 46%). ¹H NMR (400 MHz, DMSO-d⁶) δ 8.14 (d, J=8.0 Hz, 2H), 7.80 (s, 1H), 7.47 (d, J=8.0 Hz, 2H), 7.11-7.02 (m, 3H), 6.78 (s, 1H), 6.68 (s, 1H), 6.47 (s, 1H), 3.52 (s, 3H), 3.33 (s, 3H), 2.39 (s, 3H), 2.05 (s, 6H). LCMS: m/z=564 [M+H]⁺.

### Preparation 69: 2-chloro-4-(5-(2,6-dimethylphenoxy)-1-methyl-2-oxo-1,2-dihydropyridin-4-yl)-6-methyl-1,6-dihydro-7H-pyrrolo[2, 3-c],pyridin-7-one

2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c]pyridin-7-one (7.5 g, 13.31 mmol) was dissolved in ethanol (300 mL, 40 V) and water (75 mL, 10 V). Sodium hydroxide (2.66 g, 66.59 mmol) was added to the reaction mixture and resulting mixture was allowed to stir at 70° C. for 2 h. The reaction mixture was diluted with water (50 mL) and extracted by ethyl acetate (50 mL). The combined organics were dried over Na₂SO₄, filtered and evaporated. The residue was triturated with hexane (30 mL). The resulting residue was dried to give off-white solid. (4 g, 73%). ¹H NMR: (400 MHz, DMSO) δ 12.81 (s, 1H), 7.49 (d, J=7.6 Hz, 1H), 7.11 (d, J=7.2 Hz, 2H), 7.04 (t, J=2 Hz, J=8.4 Hz, 1H), 6.64 (s, 1H), 6.50 (s, 1H), 6.28 (s, 1H), 3.56 (s, 3H), 3.31 (s, 3H), 2.07 (s, 6H). LCMS: m/z=410 [M+H]⁺.

### Preparation 70: 4-(5-(2,6-dimethylphenoxy)-1-methyl-2-oxo-1,2-dihydropyridin-4-yl)-6-methyl-2-(1-(trifluoromethyl)-1H-pyrazol-4-yl)-1,6-dihydro-7H-pyrrolo[2,3-c]pyridin-7-one

2-chloro-4-(5-(2,6-dimethylphenoxy)-1-methyl-2-oxo-1,2-dihydropyridin-4-yl)-6-methyl-1,6-dihydro-7H-pyrrolo[2,3-c]pyridin-7-one (0.20 g, 0.488 mmol) was dissolved in 1,4-dioxane (12 mL) under argon. 4-(4,4,5,5-tetramethyl-1,3,2-dioxaborolan-2-yl)-1-(trifluoromethyl)-1H-pyrazole (0.51 g, 1.95 mmol) was added to the reaction mixture followed by potassium phosphate (0.31 g, 1.46 mmol) and water (2.0 mL) at room temperature. The suspension was degassed with argon for 15 min. Xphos PdG₃ (0.082 g, 0.097 mmol) was added to the reaction mixture. The reaction mixture was heated at 120° C. for 4 h. The reaction mixture was diluted with ethyl acetate (100 mL) and filtrate through celite pad. The organic phase was dried over anhydrous sodium sulphate, filtered and concentrated under reduced pressure to afford crude material. The residue was purified by reversed phase flash chromatography (C18 column; elution with 40-50% ACN in water) to afford 4-(5-(2,6-dimethylphenoxy)-1-methyl-2-oxo-1,2-dihydropyridin-4-yl)-6-methyl-2-(1-(trifluoromethyl)-1H-pyrazol-4-yl)-1,6-dihydro-7H-pyrrolo[2,3-c]pyridin-7-one (0.088 g, 35%) as a pale-yellow solid. ¹H NMR (400 MHz, DMSO-d⁶) δ 12.53 (s, 1H), 9.03 (s, 1H), 8.57 (s, 1H), 7.52 (s, 1H), 7.11-7.05 (m, 3H), 6.83 (d, J=2.0 Hz, 1H), 6.69 (s, 1H), 6.56 (s, 1H), 3.60 (s, 3H), 3.33 (s, 3H), 2.09 (s, 6H). LCMS^{XX}: m/z=510 [M+H]⁺.

### Example 12: 2-(1-(difluoromethyl)-1H-pyrazol-4-yl)-4-(5-(2,6-dimethylphenoxy)-1-methyl-2-oxo-1,2-dihydropyridin-4-yl)-6-methyl-1,6-dihydro-7H-pyrrolo[2,3-c]pyridin-7-one

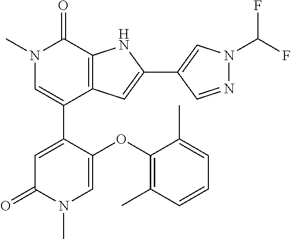 [SMILES: Cc1cccc(C)c1Oc1cn(C)c(=O)cc1-c1cn(C)c(=O)c2[nH]c(-c3cnn(C(F)F)c3)cc12]

### Preparation 71: 2-(1-(difluoromethyl)-1H-pyrazol-4-yl)-4-(5-(2,6-dimethylphenoxy)-1-methyl-2-oxo-1,2-dihydropyridin-4-yl)-6-methyl-1,6-dihydro-7H-pyrrolo[2,3-c]pyridin-7-one

Following the procedure described in preparation 70, 2-chloro-4-(5-(2,6-dimethylphenoxy)-1-methyl-2-oxo-1,2-dihydropyridin-4-yl)-6-methyl-1,6-dihydro-7H-pyrrolo[2,3-c]pyridin-7-one (0.2 g, 0.488 mmol) and 1-(difluoromethyl)-4-(4,4,5,5-tetramethyl-1,3,2-dioxaborolan-2-yl)-1H-pyrazole (0.477 g, 1.95 mmol) was reacted to give title compound (0.016 g, 6.67%) as a pale yellow solid. ¹H NMR (400 MHz, DMSO-d⁶) δ 12.48 (s, 1H), 8.80 (s, 1H), 8.38 (s, 1H), 7.88 (s, 1H), 7.51 (s, 1H), 7.12-7.03 (m, 2H), 6.74 (s, 1H), 6.68 (s, 1H), 6.56 (s, 1H), 3.60 (s, 3H), 3.37 (s, 3H), 2.09 (s, 6H). LCMS^{XX}: m/z=492 [M+H]⁺.

### Example 13: 4-(5-(2,6-dimethylphenoxy)-1-methyl-2-oxo-1,2-dihydropyridin-4-yl)-6-methyl-2-(1-(2,2,2-trifluoroethyl)-1H-pyrazol-4-yl)-1,6-dihydro-7H-pyrrolo[2,3-c]pyridin-7-one

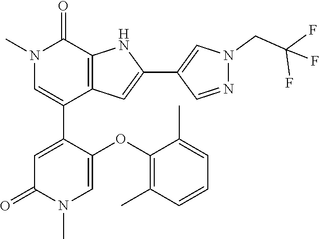 [SMILES: Cc1cccc(C)c1Oc1cn(C)c(=O)cc1-c1cn(C)c(=O)c2[nH]c(-c3cnn(CC(F)(F)F)c3)cc12]

### Preparation 72: 4-(5-(2,6-dimethylphenoxy)-1-methyl-2-oxo-1,2-dihydropyridin-4-yl)-6-methyl-2-(1-(2,2,2-trifluoroethyl)-1H-pyrazol-4-yl)-1,6-dihydro-7H-pyrrolo[2,3-c]pyridin-7-one

Following the procedure described in preparation 70, 2-chloro-4-(5-(2,6-dimethylphenoxy)-1-methyl-2-oxo-1,2-dihydropyridin-4-yl)-6-methyl-1,6-dihydro-7H-pyrrolo[2,3-c]pyridin-7-one (0.20 g, 0.48 mmol) and 4-(4,4,5,5-tetramethyl-1,3,2-dioxaborolan-2-yl)-1-(2,2,2-trifluoroethyl)-1H-pyrazole (0.53 g, 1.95 mmol) was reacted to give title compound (0.160 g, 62.63%) as an off white solid. ¹H NMR: (400 MHz, DMSO) δ 12.40 (s, 1H), 8.42 (s, 1H), 8.16 (s, 1H), 7.57 (s, 1H), 7.12-7.03 (m, 3H), 6.67 (s, 1H), 6.61 (s, 1H), 6.55 (s, 1H), 5.24-5.17 (q, J=9.2 Hz, 2H), 3.59 (s, 3H), 3.33 (s, 3H) 2.09 (s, 6H). LCMS^{XX}: m/z=524 [M+H]⁺.

### Example 14: 4-(5-(2,6-dimethylphenoxy)-1-methyl-2-oxo-1,2-dihydropyridin-4-yl)-2-(1-(2-fluoroethyl)-1H-pyrazol-4-yl)-6-methyl-1,6-dihydro-7H-pyrrolo[2,3-c]pyridin-7-one

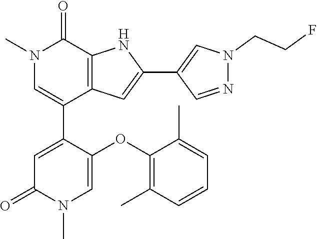 [SMILES: Cc1cccc(C)c1Oc1cn(C)c(=O)cc1-c1cn(C)c(=O)c2[nH]c(-c3cnn(CCF)c3)cc12]

### Preparation 73: 4-(5-(2,6-dimethylphenoxy)-1-methyl-2-oxo-1,2-dihydropyridin-4-yl)-2-(1-(2-fluoroethyl)-1H-pyrazol-4-yl)-6-methyl-1,6-dihydro-7H-pyrrolo[2,3-c]pyridin-7-one

Following the procedure described in preparation 70, 2-chloro-4-(5-(2,6-dimethylphenoxy)-1-methyl-2-oxo-1,2-dihydropyridin-4-yl)-6-methyl-1,6-dihydro-7H-pyrrolo[2,3-c]pyridin-7-one (0.20 g, 0.48 mmol) and 1-(2-fluoroethyl)-4-(4,4,5,5-tetramethyl-1,3,2-dioxaborolan-2-yl)-1H-pyrazole (0.45 g, 1.95 mmol) was reacted to give title compound (0.15 g, 63%) as an off white solid. ¹H NMR: (400 MHz, DMSO) δ 12.31 (s, 1H), 8.34 (s, 1H), 8.07 (s, 1H), 7.49 (s, 1H), 7.12-7.04 (m, 3H), 6.67 (s, 1H), 6.56-6.54 (m, 2H), 4.84 (t, J=4.8 Hz, 1H), 4.72 (t, J=4.8 Hz, 1H), 4.46 (dt, J=23.2, 4.6 Hz, 2H), 3.58 (s, 3H), 3.33 (s, 3H), 2.09 (s, 6H). LCMS^{XX}: m/z=488 [M+H]⁺.

### Example 15: 4-(5-(2,6-dimethylphenoxy)-1-methyl-2-oxo-1,2-dihydropyridin-4-yl)-6-methyl-2-(3-methyl-1-(2,2,2-trifluoroethyl)-1H-pyrazol-4-yl)-1,6-dihydro-7H-pyrrolo[2,3-c]pyridin-7-one

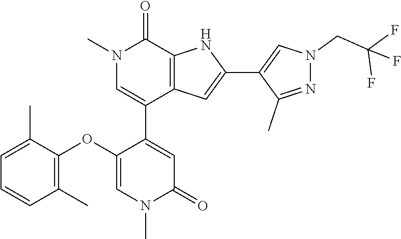 [SMILES: Cc1cccc(C)c1Oc1cn(C)c(=O)cc1-c1cn(C)c(=O)c2[nH]c(-c3cn(CC(F)(F)F)nc3C)cc12]

### Preparation 74: 4-(5-(2,6-dimethylphenoxy)-1-methyl-2-oxo-1,2-dihydropyridin-4-yl)-6-methyl-2-(3-methyl-1-(2,2,2-trifluoroethyl)-1H-pyrazol-4-yl)-1,6-dihydro-7H-pyrrolo[2, 3-c]pyridin-7-one

Following the procedure described in preparation 70, 2-chloro-4-(5-(2,6-dimethylphenoxy)-1-methyl-2-oxo-1,2-dihydropyridin-4-yl)-6-methyl-1,6-dihydro-7H-pyrrolo[2,3-c]pyridin-7-one (0.20 g, 0.48 mmol) and 3-methyl-4-(4,4,5,5-tetramethyl-1,3,2-dioxaborolan-2-yl)-1-(2,2,2-trifluoroethyl)-1H-pyrazole (0.56 g, 1.95 mmol) was reacted to give title compound (0.065 g, 24%) as an brown liquid. ¹H NMR: (400 MHz, DMSO-d⁶) δ 12.27 (s, 1H), 8.39 (s, 1H), 7.48 (s, 1H), 7.11-7.02 (m, 3H), 6.66 (s, 1H), 6.53 (s, 1H), 6.39 (s, 1H), 5.12 (q, J=9.2 Hz, 2H), 3.59 (s, 3H), 3.32 (s, 3H), 2.35 (s, 3H), 2.10 (d, J=10.8 Hz, 6H). LCMS^{XX}: m/z=538 [M+H]⁺.

### Example 16: 4-(5-(2,6-dimethylphenoxy)-1-methyl-2-oxo-1,2-dihydropyridin-4-yl)-6-methyl-2-(5-methyl-1-(2,2,2-trifluoroethyl)-1H-pyrazol-4-yl)-1,6-dihydro-7H-pyrrolo[2,3-c]pyridin-7-one

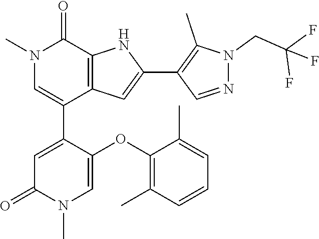 [SMILES: Cc1cccc(C)c1Oc1cn(C)c(=O)cc1-c1cn(C)c(=O)c2[nH]c(-c3cnn(CC(F)(F)F)c3C)cc12]

### Preparation 75: 4-(5-(2,6-dimethylphenoxy)-1-methyl-2-oxo-1,2-dihydropyridin-4-yl)-6-methyl-2-(5-methyl-1-(2,2,2-trifluoroethyl)-1H-pyrazol-4-yl)-1,6-dihydro-7H-pyrrolo[2, 3-c]pyridin-7-one

Following the procedure described in preparation 70, 2-chloro-4-(5-(2,6-dimethylphenoxy)-1-methyl-2-oxo-1,2-dihydropyridin-4-yl)-6-methyl-1,6-dihydro-7H-pyrrolo[2,3-c]pyridin-7-one (0.20 g, 0.48 mmol) and 3-methyl-4-(4,4,5,5-tetramethyl-1,3,2-dioxaborolan-2-yl)-1-(2,2,2-trifluoroethyl)-1H-pyrazole (0.56 g, 1.95 mmol) was reacted to give title compound (0.095 g, 36%) as an brown liquid. ¹H NMR: (400 MHz, DMSO-d⁶) δ 12.27 (s, 1H), 8.35 (s, 1H), 7.49 (d, J=4 Hz, 1H), 7.10-7.02 (m, 3H), 6.66 (s, 1H), 6.53. (d, J=2.8 Hz, 1H), 6.39 (s, 1H), 5.20-5.09 (m, 2H), 3.59 (s, 3H), 3.32 (s, 3H), 2.35 (s, 3H), 2.09 (s, 6H). LCMS^{XX}: m/z=538 [M+H]⁺.

### Example 17: 6-methyl-4-(1-methyl-2-oxo-5-(3-((trifluoromethoxy)methyl)phenyl)-1,2-dihydropyridin-4-yl)-2-(1-(trifluoromethyl)-1H-pyrazol-4-yl)-1,6-dihydro-7H-pyrrolo[2,3-c]pyridin-7-one

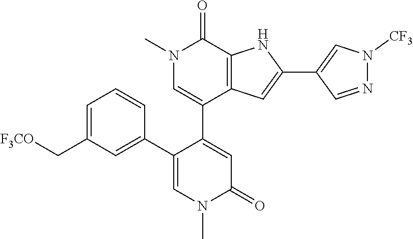 [SMILES: Cn1cc(-c2cccc(COC(F)(F)F)c2)c(-c2cn(C)c(=O)c3[nH]c(-c4cnn(C(F)(F)F)c4)cc23)cc1=O]

### Preparation 76: 4-brono-2-iodo-7-methoxy-1-tosyl-1H-pyrrolo[2,3-c]pyridine

4-bromo-7-methoxy-1-tosyl-1H-pyrrolo[2, 3-c]pyridine (200 g, 526.3 mmol) was dissolved in dry THF (4000 mL, 20V) at room temperature. LDA (1M in THF) (421 mL, 421 mmol) was dropwise added to the reaction mixture at −78° C. for 30 min. The resulting reaction mixture was allowed to stir at −78° C. for 2 h. Iodine (213.7 g, 842 mmol) in dry THF (1000 mL) was dropwise added to the reaction mixture at −78° C. for 15 min. The resulting reaction mixture was allowed to stir at −78° C. to rt for 4 h. The reaction mixture was quenched in NH₄Cl solution (3000 mL) and filtered through celite bed. The filtrate was washed with ethyl acetate (3×2000 mL). The organic fraction was washed with sodium thiosulphate and dried over Na₂SO₄, filtered and evaporated to yield 4-bromo-2-iodo-7-methoxy-1-tosyl-1H-pyrrolo[2, 3-c]pyridine (100 g, 37.34%) as a white solid. ¹H NMR: (400 MHz, DMSO-d6) δ 7.99 (s, 1H), 7.91 (d, J=8.4 Hz, 2H), 7.52 (d, J=8.0 Hz, 2H), 7.19 (s, 1H), 3.81 (s, 3H), 2.38 (s, 3H). LCMS: m/z=509 [M+H]⁺.

### Preparation 77: 4-bromo-2-iodo-1-tosyl-1,6-dihydro-7H-pyrrolo[2,3-c]pyridin-7-one

4-bromo-2-iodo-7-methoxy-1-tosyl-1H-pyrrolo[2,3-c]pyridine (298 g, 589 mmol) was dissolved in acetonitrile (2980 mL, 10V) at room temperature. Sodium iodide (132.4 g, 883 mmol) was added to the reaction mixture and allowed stir at room temperature for 15 minutes. The resulting solution was cooled at 0° C. and dropwise added Trimethylsilyl chloride (112 mL, 883.6 mmol). The resulting mixture was allowed to stir at ambient temperature for 1 h. Water (149 mL, 0.5V) was added to the reaction mixture and heated at 65° C. for 3 h. The resulting mixture was quenched with ice-cold water (3000 mL) and stirred for 30 min. The resulting residue was filtered and triturated by n-hexane (700 mL) and diethyl ether (700 mL). The solid was dried overnight under vacuum at 45° C. to give white solid (250 g, 86.28%). ¹H NMR (400 MHz, DMSO d6) δ 11.52 (s, 1H), 8.07 (d, J=8.4 Hz, 2H), 7.47 (d, J=8.4 Hz, 2H), 7.37 (s, 1H), 6.99 (s, 1H), 2.38 (s, 3H). LCMS: m/z=495 [M+H]⁺.

### Preparation 78: 4-bromo-2-iodo-6-methyl-1-tosyl-1,6-dihydro-7H-pyrrolo[2,3-c]pyridin-7-one

4-bromo-2-iodo-1-tosyl-1,6-dihydro-7H-pyrrolo[2,3-c]pyridin-7-one (250 g, 508.2 mmol) was dissolved in DMF (5000 mL, 20V) at room temperature. Potassium carbonate (140 g, 1016 mmol) was added to the reaction mixture at 0° C. and the resulting solution was allowed to stir at same temperature for 30 min. Methyl iodide (47.9 mL, 762 mmol) was dropwise added to the reaction mixture at 0° C. and allows to stir at room temperature for 4 h. The reaction mixture was quenched with ice cold water (2500 mL) to afford yellow coloured precipitate. The resulting precipitate was filtered and washed with water (1000 mL) and hexane (1000 mL). The solid was dried overnight under vacuum at 45° C. to give white solid (190 g, 73.90%). ¹H NMR: (400 MHz, DMSO-d6) δ 8.09 (d, J=8.4 Hz, 2H), 7.8 (s, 1H), 7.49 (d, J=8.0 Hz, 2H), 6.95 (s, 1H), 3.39 (s, 3H), 2.41 (s, 3H). LCMS: m/z=509 [M+H]⁺.

### Preparation 79: 4-bromo-6-methyl-1-tosyl-2-(1-(trifluoromethyl)-1H-pyrazol-4-yl)-1,6-dihydro-7H-pyrrolo[2, 3-c]pyridin-7-one

To a stirred mixture of 4-bromo-2-iodo-6-methyl-1-tosyl-1,6-dihydro-7H-pyrrolo[2,3-c]pyridin-7-one (5.00 g, 9.86 mmol, 1.00 equiv) and 4-(4,4,5,5-tetramethyl-1,3,2-dioxaborolan-2-yl)-1-(trifluoromethyl)-1H-pyrazole (9.04 g, 34.5 mmol, 3.50 equiv) in dioxane (100 mL) and H₂O (10 mL) at room temperature was added K₃PO₄ (4.19 g, 19.7 mmol, 2.00 equiv) and Pd₂(dba)₃ (900 mg, 0.986 mmol, 0.100 equiv). The resulting mixture was stirred at 50° C. under nitrogen atmosphere overnight. The reaction was quenched with water at room temperature. The resulting mixture was extracted with EtOAc (3×50 mL). The combined organic layers were washed with brine, dried over anhydrous Na₂SO₄. After filtration, the combined filtrate was concentrated under reduced pressure. The residue was purified by silica gel column chromatography, eluted with PE/EA (1:2) to afford the crude compound as a yellow solid (3.5 g, crude). The crude product was further purified by Prep-HPLC^{W} to afford the title product as a white solid (2.10 g, 41.3%). ¹H NMR (400 MHz, DMSO-d⁶) δ 8.88 (s, 1H), 8.20 (s, 1H), 7.93-7.78 (m, 3H), 7.41 (d, J=8.2 Hz, 2H), 6.78 (d, J=1.2 Hz, 1H), 3.40 (s, 3H), 1.99 (s, 3H). LCMS: m/z=517 [M+H]⁺.

### Preparation 80: (6-methyl-7-oxo-1-tosyl-2-(1-(trifluoromethyl)-1H-pyrazol-4-yl)-6,7-dihydro-1H-pyrrolo[2,3-c]pyridin-4-yl)boronic acid

To a stirred mixture of 4-bromo-6-methyl-1-(4-methylbenzenesulfonyl)-2-[1-(trifluoromethyl)pyrazol-4-yl]pyrrolo[2,3-c]pyridin-7-one (2.05 g, 3.98 mmol, 1.00 equiv) and 4,4,4′,4′,5,5,5′,5′-octamethyl-2,2′-bi(1,3,2-dioxaborolane) (5.39 g, 23.9 mmol, 6.00 equiv) in THF (100 mL) at room temperature was added AcOK (0.780 g, 7.96 mmol, 2.00 equiv) and Pd(PPh₃)₂Cl₂ (0.280 g, 0.398 mmol, 0.100 equiv). The resulting mixture was stirred overnight at 60° C. under nitrogen atmosphere. The reaction was quenched with water at room temperature. The resulting mixture was extracted with EtOAc (3×50 mL). The combined organic layers were washed with brine, dried over anhydrous Na₂SO₄. After filtration, the filtrate was concentrated under reduced pressure. The residue was purified by silica gel column chromatography, eluted with PE/EA (1:1) to afford the crude product (2.03 g, yellow solid). The residue was further purified by Prep-HPLC^{X} to afford the title compound as a light-yellow solid (1.16 g, 61%). ¹H NMR (400 MHz, DMSO-d⁶) δ 8.82 (s, 1H), 8.14 (s, 1H), 8.00 (s, 2H), 7.86-7.77 (m, 3H), 7.38 (d, J=8.1 Hz, 2H), 7.04 (s, 1H), 3.41 (s, 3H), 2.38 (s, 3H). LCMS: m/z=481 [M+H]⁺.

### Preparation 81: 4-bromo-5-(3-(hydroxymethyl)phenyl)-1-methylpyridin-2 (1H)-one

To a stirred mixture of 4-bromo-5-iodo-1-methylpyridin-2 (1H)-one (5.00 g, 15.9 mmol, 1.00 equiv) and 3-(hydroxymethyl)phenylboronic acid (3.63 g, 23.9 mmol, 1.50 equiv) in DMF (100 mL) and H₂O (10 mL) were added Na₂CO₃ (3.38 g, 31.9 mmol, 2.00 equiv) and Pd(PPh₃)₄ (1.84 g, 1.59 mmol, 0.100 equiv) at room temperature. The resulting mixture was stirred at 100° C. under nitrogen atmosphere for 5 h. The reaction was quenched with water at room temperature. The resulting mixture was extracted with EtOAc (3×60 mL). The combined organic layers were washed with brine, dried over anhydrous Na₂SO₄. After filtration, the filtrate was concentrated under reduced pressure. The residue was purified by silica gel column chromatography, eluted with EA/MeOH (10:1) to afford the title compound as a yellow solid (5.87 g, crude). LCMS: m/z=296 [M+H]⁺.

### Preparation 82: 4-bromo-5-(3-(bromomethyl)phenyl)-1-methylpyridin-2 (1H)-one

To a stirred solution of 4-bromo-5-(3-(hydroxymethyl)phenyl)-1-methylpyridin-2 (1H)-one (5.80 g, 19.7 mmol, 1.00 equiv) in DCM (100 mL) at 0° C. under nitrogen atmosphere was added PBr₃ (5.87 g, 21.7 mmol, 1.10 equiv) dropwise. The resulting mixture was stirred at 0° C. under nitrogen atmosphere for 1 h. The reaction was quenched with water/ice at 0° C. The resulting mixture was extracted with EtOAc (3×50 mL). The combined organic layers were washed with brine, dried over anhydrous Na₂SO₄. After filtration, the filtrate was concentrated under reduced pressure. The residue was purified by silica gel column chromatography, eluted with EA/MeOH (10:1) to afford the crude product (4.0 g) as a yellow solid. The crude product was further purified by Prep-HPLC^{Y} to afford the title compound as a light-yellow solid (1.00 g, 14%). LCMS: m/z=358 [M+H]⁺.

### Preparation 83: 4-bromo-1-methyl-5-(3-((trifluoromethoxy)methyl)phenyl)pyridin-2 (1H)-one

To a solution of 4-bromo-5-(3-(bromomethyl)phenyl)-1-methylpyridin-2 (1H)-one (1.00 g, 2.80 mmol, 1.00 equiv) in MeCN (5 mL) was added AgF (1.00 g, 7.87 mmol, 2.81 equiv). The mixture was stirred at room temperature under nitrogen atmosphere for 30 min. To the above mixture was added trifluoromethyl trifluoromethanesulfonate (0.90 g, 4.12 mmol, 1.47 equiv) dropwise at −30° C. The resulting mixture was stirred overnight at room temperature. The resulting mixture was filtered through a short pad of Celite. The pad was washed with MeCN. The combined filtrate was concentrated under reduced pressure. The residue was purified by Prep-HPLC^{Z} to afford the title compound as a yellow solid (610 mg, 60%). LCMS: m/z=364 [M+H]⁺.

### Preparation 84: 6-methyl-4-(1-methyl-2-oxo-5-(3-((trifluoromethoxy)methyl)phenyl)-1,2-dihydropyridin-4-yl)-1-tosyl-2-(1-(trifluoromethyl)-1H-pyrazol-4-yl)-1,6-dihydro-7H-pyrrolo[2,3-c]pyridin-7-one

To a stirred mixture of 4-bromo-1-methyl-5-(3-((trifluoromethoxy)methyl)phenyl)pyridin-2 (1H)-one (497 mg, 1.37 mmol, 1.20 equiv) and (6-methyl-7-oxo-1-tosyl-2-(1-(trifluoromethyl)-1H-pyrazol-4-yl)-6,7-dihydro-1H-pyrrolo[2,3-c]pyridin-4-yl)boronic acid (550 mg, 1.15 mmol, 1.00 equiv) in DME (10.0 mL) and H₂O (2.0 mL) were added Na₂CO₃ (243 mg, 2.29 mmol, 2.00 equiv) and Pd(dppf)Cl₂·CH₂Cl₂ (83.8 mg, 0.115 mmol, 0.100 equiv) at room temperature under nitrogen atmosphere. The resulting mixture was stirred for 3 h at 60° C. The reaction was quenched with water at room temperature. The resulting mixture was extracted with EtOAc (3×10.0 mL). The combined organic layers were washed with brine, dried over anhydrous Na₂SO₄. After filtration, the filtrate was concentrated under reduced pressure. The residue was purified by silica gel column chromatography, eluted with EA/MeOH (8:1) to afford the title compound as a light-yellow solid (488 mg, 59%). LCMS: m/z=718 [M+H]⁺.

### Preparation 85: 6-methyl-4-(1-methyl-2-oxo-5-(3-((trifluoromethoxy)methyl)phenyl)-1,2-dihydropyridin-4-yl)-2-(1-(trifluoromethyl)-1H-pyrazol-4-yl)-1,6-dihydro-7H-pyrrolo[2,3-c]pyridin-7-one

Following the procedure described in preparation 7, 6-methyl-4-(1-methyl-2-oxo-5-(3-((trifluoromethoxy)methyl)phenyl)-1,2-dihydropyridin-4-yl)-1-tosyl-2-(1-(trifluoromethyl)-1H-pyrazol-4-yl)-1,6-dihydro-7H-pyrrolo[2,3-c]pyridin-7-one (468 mg, 0.652 mmol, 1.00 equiv) was reacted to give the crude compound which was purified by Prep-HPLC^{AA} to afford the title compound as a white solid (162 mg, 44%). ¹H NMR (400 MHz, DMSO-d⁶) δ 12.27 (s, 1H), 8.86 (s, 1H), 8.41 (s, 1H), 7.91 (s, 1H), 7.35 (d, J=1.9 Hz, 1H), 7.26-7.07 (m, 4H), 6.50 (s, 1H), 6.28 (s, 1H), 5.00 (s, 2H), 3.56 (s, 3H), 3.44 (s, 3H). LCMS^{XX}: m/z=564 [M+H]⁺.

### Example 18: 6-methyl-4-(1-methyl-2-oxo-5-(4-((trifluoromethoxy)methyl)phenyl)-1,2-dihydropyridin-4-yl)-2-(1-(trifluoromethyl)-1H-pyrazol-4-yl)-1,6-dihydro-7H-pyrrolo[2,3-c]pyridin-7-one

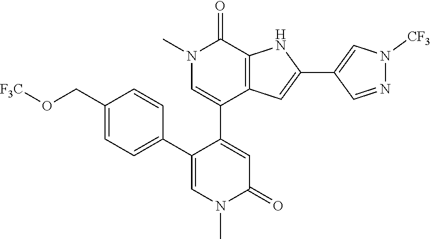 [SMILES: Cn1cc(-c2ccc(COC(F)(F)F)cc2)c(-c2cn(C)c(=O)c3[nH]c(-c4cnn(C(F)(F)F)c4)cc23)cc1=O]

### Preparation 86: 4-bromo-5-(4-(hydroxymethyl)phenyl)-1-methylpyridin-2 (1H)-one

To a stirred mixture of 4-bromo-5-iodo-1-methylpyridin-2 (1H)-one (5.00 g, 15.9 mmol, 1.00 equiv) and 4-(hydroxymethyl)phenylboronic acid (3.63 g, 23.9 mmol, 1.50 equiv) in DME (100 mL) and H₂O (10 mL) at room temperature were added Na₂CO₃ (3.38 g, 31.9 mmol, 2.00 equiv) and Pd(PPh₃)₄ (1.84 g, 1.59 mmol, 0.100 equiv). The resulting mixture was stirred at 100° C. under nitrogen atmosphere for 4 h. The reaction was quenched with water at room temperature. The resulting mixture was extracted with EtOAc (3×150 mL). The combined organic layers were washed with brine, dried over anhydrous Na₂SO₄. After filtration, the combined filtrate was concentrated under reduced pressure. The residue was purified by silica gel column chromatography, eluted with EA/MeOH (10:1) to afford the title compound as a yellow solid (2.80 g, 60%). LCMS: m/z=296 [M+H]⁺.

### Preparation 87: 4-bromo-5-(4-(bromomethyl)phenyl)-1-methylpyridin-2 (1H)-one

To a stirred solution of 4-bromo-5-(4-(hydroxymethyl)phenyl)-1-methylpyridin-2 (1H)-one (2.40 g, 8.16 mmol, 1.00 equiv) in DCM (40.0 mL) at 0° C. under nitrogen atmosphere was added PBr₃ (2.25 g, 8.98 mmol, 1.10 equiv) dropwise. The resulting mixture was stirred at 0° C. for 30 min. The reaction at 0° C. was quenched with water. The resulting mixture was extracted with CH₂Cl₂ (3×20 mL). The combined organic layers were washed with brine, dried over anhydrous Na₂SO₄. After filtration, the filtrate was concentrated under reduced pressure. This resulted in the title compound as a yellow solid (2.80 g, crude). LCMS: m/z=358 [M+H]⁺.

### Preparation 88: 4-bromo-1-methyl-5-(4-((trifluoromethoxy)methyl)phenyl)pyridin-2 (1H)-one

To a solution of 4-bromo-5-(4-(bromomethyl)phenyl)-1-methylpyridin-2 (1H)-one (3.0 g, 8.40 mmol, 1.00 equiv) in CH₃CN (30 mL) was added AgF (6.0 g, 47.3 mmol, 5.63 equiv). The mixture was stirred at room temperature under nitrogen atmosphere for 30 min. To the above mixture at −30° C. was added trifluoromethyl trifluoromethanesulfonate (3.0 mL) dropwise. The resulting mixture was stirred at room temperature under nitrogen atmosphere overnight. The reaction was quenched with water. The resulting mixture was extracted with EtOAc (3×50 mL). The combined organic layers were washed with water, dried over anhydrous Na₂SO₄. After filtration, the filtrate was concentrated under reduced pressure. The residue was purified by silica gel column chromatography, eluted with PE/EA (1:2) to afford the title compound as a yellow solid (420 mg, 14%). ¹H NMR (400 MHz, DMSO-d⁶) δ 7.89 (s, 1H), 7.52-7.47 (m, 2H), 7.46-7.40 (m, 2H), 6.89 (s, 1H), 5.22 (s, 2H), 3.45 (s, 3H). LCMS: m/z=364 [M+H]⁺.

### Preparation 89: 6-methyl-4-(1-methyl-2-oxo-5-(4-((trifluoromethoxy)methyl)phenyl)-1,2-dihydropyridin-4-yl)-1-tosyl-2-(1-(trifluoromethyl)-1H-pyrazol-4-yl)-1,6-dihydro-7H-pyrrolo[2,3-c]pyridin-7-one

To a stirred mixture of (6-methyl-7-oxo-1-tosyl-2-(1-(trifluoromethyl)-1H-pyrazol-4-yl)-6,7-dihydro-1H-pyrrolo[2,3-c]pyridin-4-yl)boronic acid (510 mg, 0.930 mmol, 1.00 equiv) and 4-bromo-1-methyl-5-(4-((trifluoromethoxy)methyl)phenyl)pyridin-2 (1H)-one (420 mg, 1.16 mmol, 1.25 equiv) in DME (5.0 mL) and H₂O (1.0 mL) at room temperature were added Na₂CO₃ (197 mg, 1.86 mmol, 2.00 equiv) and Pd(dppf)Cl₂·CH₂Cl₂ (75.8 mg, 0.0930 mmol, 0.100 equiv). The resulting mixture was stirred at 60° C. under nitrogen atmosphere for 2 h. The reaction was quenched with water at room temperature. The resulting mixture was extracted with EtOAc (3×5 mL). The combined organic layers were washed with brine, dried over anhydrous Na₂SO₄. After filtration, the filtrate was concentrated under reduced pressure. This resulted in the title product as a yellow solid (880 mg, crude). LCMS: m/z=718 [M+H]⁺.

### Preparation 90: 6-methyl-4-(1-methyl-2-oxo-5-(4-((trifluoromethoxy)methyl)phenyl)-1,2-dihydropyridin-4-yl)-2-(1-(trifluoromethyl)-1H-pyrazol-4-yl)-1,6-dihydro-7H-pyrrolo[2,3-c]pyridin-7-one

Following the procedure described in preparation 7, 6-methyl-4-(1-methyl-2-oxo-5-(4-((trifluoromethoxy)methyl)phenyl)-1,2-dihydropyridin-4-yl)-1-tosyl-2-(1-(trifluoromethyl)-1H-pyrazol-4-yl)-1,6-dihydro-7H-pyrrolo[2,3-c]pyridin-7-one (880 mg, crude, 1.23 mmol, 1.00 equiv) was reacted to give the crude product which was purified by Prep-HPLC^{AB} to afford the title compound as a white solid (100 mg, 17%). ¹H NMR (400 MHz, DMSO-d⁶) δ 12.31 (s, 1H), 8.87 (s, 1H), 8.42 (s, 1H), 7.92 (s, 1H), 7.24 (s, 4H), 7.11 (s, 1H), 6.41 (d, J=72.9 Hz, 2H), 5.01 (s, 2H), 3.72-3.38 (m, 6H). LCMS^{L}: m/z=564 [M+H]⁺.

### Example 19: 6-ethyl-4-(1-methyl-2-oxo-5-phenyl-1,2-dihydropyridin-4-yl)-2-(1-(trifluoromethyl)-1H-pyrazol-4-yl)-1,6-dihydro-7H-pyrrolo[2,3-c]pyridin-7-one

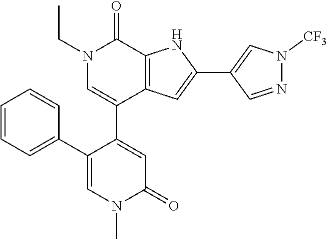 [SMILES: CCn1cc(-c2cc(=O)n(C)cc2-c2ccccc2)c2cc(-c3cnn(C(F)(F)F)c3)[nH]c2c1=O]

### Preparation 91: 4-bromo-2-chloro-6-ethyl-1-tosyl-1, 6-dihydro-7H-pyrrolo[2, 3-c]pyridin-7-one

4-bromo-2-chloro-1-tosyl-1, 6-dihydro-7H-pyrrolo[2, 3-c]pyridin-7-one (25 g, 62.51 mmol) was dissolved in DMF (500 mL) at room temperature. Potassium carbonate (17.27 g, 125.02 mmol) was portion wise added to the reaction mixture at 0° C. and the resulting solution was allowed to stir at same temperature for 30 min. Methyl iodide (7.53 mL, 14.62 g, 93.76 mmol) was dropwise added to the reaction mixture at 0° C. and allows to stir at room temperature for 4 h. The reaction mixture was quenched with ice cold water (500 mL) to afford brown coloured precipitate was purified by reverse phase chromatography, eluting at 60-100% acetonitrile in water. Fraction corresponding to product were collected and evaporated to get brown solid was triturated by diethyl ether (100 ml) to afford brown solid (7.5 g, 31%). ¹H NMR: (400 MHz, DMSO-d6) δ 8.12 (d, J=8.4 Hz, 2H), 7.91 (s, 1H), 7.50 (d, J=8.4 Hz, 2H), 6.80 (s, 1H), 3.92 (q, J=14 Hz, 2H), 2.41 (s, 3H), 1.16 (t, J=7.2 Hz, 3H). LCMS: m/z=431 [M+H]⁺

### Preparation 92: 2-chloro-4-(5,5-dimethyl-1,3,2-dioxaborinan-2-yl)-6-ethyl-1-tosyl-1,6-dihydro-7H-pyrrolo[2,3-c]pyridin-7-one

To a stirred solution of 4-bromo-2-chloro-6-ethyl-1-tosyl-1,6-dihydro-7H-pyrrolo[2,3-c]pyridin-7-one (3.00 g, 6.98 mmol, 1.00 equiv) and 2-(5,5-dimethyl-1,3,2-dioxaborinan-2-yl)-5,5-dimethyl-1,3,2-dioxaborinane (9.46 g, 41.9 mmol, 6.00 equiv) in THF (150 mL) at room temperature under nitrogen atmosphere were added KOAc (1.37 g, 14.0 mmol, 2.00 equiv) and Pd(PPh₃)₂Cl₂ (0.490 g, 0.698 mmol, 0.100 equiv). The mixture was stirred at 60° C. overnight. The residue was diluted with water (100 mL). The aqueous layer was extracted with EtOAC (3×100 mL). The combined organic layers were washed with brine, dried over anhydrous Na₂SO₄. After filtration, the filtrate was concentrated under reduced pressure. The residue was purified by silica gel column chromatography, eluted with PE/EA (2:1) to afford the title compound as yellow solid (2.5 g, crude). LCMS: m/z=395 [M+H]⁺ (mass of corresponding boronic acid seen under LCMS conditions).

### Preparation 93: 2-chloro-6-ethyl-4-(1-methyl-2-oxo-5-phenyl-1,2-dihydropyridin-4-yl)-1-tosyl-1,6-dihydro-7H-pyrrolo[2, 3-c]pyridin-7-one

To a stirred solution of 2-chloro-4-(5,5-dimethyl-1,3,2-dioxaborinan-2-yl)-6-ethyl-1-tosyl-1,6-dihydro-7H-pyrrolo[2,3-c]pyridin-7-one (2.02 g, 5.11 mmol, 1.80 equiv) and 4-bromo-1-methyl-5-phenylpyridin-2 (1H)-one (0.75 g, 2.84 mmol, 1.00 equiv) in DME (20.0 mL) and H₂O (4.0 mL) at room temperature under nitrogen atmosphere was added Pd(dppf)Cl₂·CH₂Cl₂ (0.231 g, 0.284 mmol, 0.100 equiv) and Na₂CO₃ (0.600 g, 5.68 mmol, 2.00 equiv). The resulting mixture was stirred at 60° C. overnight. The reaction was quenched with water (20 mL). The aqueous layer was extracted with EtOAc (3×20 mL). The combined organic layers were washed with brine, dried over anhydrous Na₂SO₄. After filtration, the filtrate was concentrated under reduced pressure. The residue was purified by silica gel column chromatography, eluted with EtOAc to afford the title compound as a yellow solid (500 mg, 33%). LCMS: m/z=534 [M+H]⁺.

### Preparation 94: 6-ethyl-4-(1-methyl-2-oxo-5-phenyl-1,2-dihydropyridin-4-yl)-1-tosyl-2-(1-(trifluoromethyl)-1H-pyrazol-4-yl)-1,6-dihydro-7H-pyrrolo[2,3-c]pyridin-7-one

Following the procedure described in preparation 8, 2-chloro-6-ethyl-4-(1-methyl-2-oxo-5-phenyl-1,2-dihydropyridin-4-yl)-1-tosyl-1,6-dihydro-7H-pyrrolo[2,3-c]pyridin-7-one (500 mg, 0.936 mmol, 1.00 equiv) and 4-(4,4,5,5-tetramethyl-1,3,2-dioxaborolan-2-yl)-1-(trifluoromethyl)pyrazole (368 mg, 1.40 mmol, 1.50 equiv) was reacted to give title compound as brown solid (570 mg, crude). LCMS: m/z=634 [M+H]⁺.

### Preparation 95: 6-ethyl-4-(1-methyl-2-oxo-5-phenyl-1,2-dihydropyridin-4-yl)-2-(1-(trifluoromethyl)-1H-pyrazol-4-yl)-1,6-dihydro-7H-pyrrolo[2,3-c]pyridin-7-one

Following the procedure described in preparation 7, 6-ethyl-4-(1-methyl-2-oxo-5-phenyl-1,2-dihydropyridin-4-yl)-1-tosyl-2-(1-(trifluoromethyl)-1H-pyrazol-4-yl)-1,6-dihydro-7H-pyrrolo[2,3-c]pyridin-7-one (570 mg, 0.900 mmol, 1.00 equiv) was reacted to give the crude compound which was purified by Prep-HPLC^{AC} to afford the title compound as white solid (63.9 mg, 15%). ¹H NMR (400 MHz, Methanol-d⁴) δ 8.58 (s, 1H), 8.20 (s, 1H), 7.81 (s, 1H), 7.22-7.19 (m, 5H), 6.84 (s, 1H), 6.79 (s, 1H), 6.49 (s, 1H), 3.93-3.92 (m, 2H), 3.70 (s, 3H), 1.08 (t, J=7.2 Hz, 3H). LCMS^{M}: m/z=480 [M+H]⁺.

### Example 20: 3,6-dimethyl-4-(1-methyl-2-oxo-5-phenyl-1,2-dihydropyridin-4-yl)-2-(1-(trifluoromethyl)-1H-pyrazol-4-yl)-1,6-dihydro-7H-pyrrolo[2,3-c]pyridin-7-one

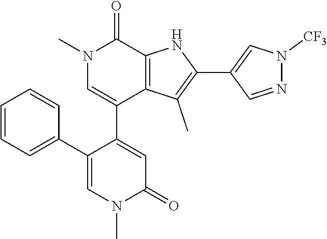 [SMILES: Cc1c(-c2cnn(C(F)(F)F)c2)[nH]c2c(=O)n(C)cc(-c3cc(=O)n(C)cc3-c3ccccc3)c12]

### Preparation 96: 3-bromo-6-methyl-4-(1-methyl-2-oxo-5-phenyl-1,2-dihydropyridin-4-yl)-2-(1-(trifluoromethyl)-1H-pyrazol-4-yl)-1,6-dihydro-7H-pyrrolo[2,3-c]pyridin-7-one

To a stirred mixture of 6-methyl-4-(1-methyl-2-oxo-5-phenyl-1,2-dihydropyridin-4-yl)-2-(1-(trifluoromethyl)-1H-pyrazol-4-yl)-1,6-dihydro-7H-pyrrolo[2,3-c]pyridin-7-one (500 mg, 1.07 mmol, 1.00 equiv) in DMF (8.0 mL) at 0° C. was added NBS (191 mg, 1.07 mmol, 1.00 equiv). The resulting mixture at room temperature was stirred for 30 min. The reaction was quenched with water. The product was precipitated by the addition of water. After filtration, the filter cake was purified by trituration with water (20 mL) to afford the title compound as a white solid (500 mg, 86%). 1 NMR (400 MHz, DMSO-d⁶) δ 12.83 (s, 1H), 8.91 (s, 1H), 8.53 (s, 1H), 7.80 (s, 1H), 7.22-7.10 (m, 6H), 6.42 (s, 1H), 3.56 (s, 3H), 3.50 (s, 3H). LCMS: m/z=546 [M+H]⁺.

### Preparation 97: 3,6-dimethyl-4-(1-methyl-2-oxo-5-phenyl-1,2-dihydropyridin-4-yl)-2-(1-(trifluoromethyl)-1H-pyrazol-4-yl)-1,6-dihydro-7H-pyrrolo[2,3-c]pyridin-7-one

To a mixture of 3-bromo-6-methyl-4-(1-methyl-2-oxo-5-phenyl-1,2-dihydropyridin-4-yl)-2-(1-(trifluoromethyl)-1H-pyrazol-4-yl)-1,6-dihydro-7H-pyrrolo[2,3-c]pyridin-7-one (380 mg, 0.698 mmol, 1.00 equiv) and 2,4,6-trimethyl-1,3,5,2,4,6-trioxatriborinane (175 mg, 1.40 mmol, 2.00 equiv) in DMF (10.0 mL) at room temperature under nitrogen atmosphere were added Na₂CO₃ (148 mg, 1.40 mmol, 2.00 equiv) and Pd(PPh₃)₄ (80.7 mg, 0.0700 mmol, 0.100 equiv). The resulting mixture was stirred at 140° C. overnight. The reaction was quenched with water at room temperature. The resulting mixture was extracted with EtOAc (3×20.0 mL). The combined organic layers were washed with brine, dried over anhydrous Na₂SO₄. After filtration, the filtrate was concentrated under reduced pressure. The resulting mixture was purified by Prep-HPLC^{AD} to afford the crude product (100 mg). The crude product was further purified by Prep-CHIRAL-HPLC^{AE} to afford the title product as a white solid (32.4 mg, 10%). ¹H NMR (400 MHz, Chloroform-d) δ 12.05 (s, 1H), 8.22 (d, J=8.7 Hz, 2H), 7.39 (s, 1H), 7.18-7.11 (m, 3H), 7.10-7.03 (m, 2H), 6.67 (d, J=14.1 Hz, 2H), 3.69 (s, 3H), 3.57 (s, 3H), 2.05 (s, 3H). LCMS^{N}: m/z=480 [M+H]⁺.

### Example 21 (Compound D): 3-fluoro-6-methyl-4-(1-methyl-2-oxo-5-phenyl-1,2-dihydropyridin-4-yl)-2-(1-(trifluoromethyl)-1H-pyrazol-4-yl)-1,6-dihydro-7H-pyrrolo[2,3-c]pyridin-7-one

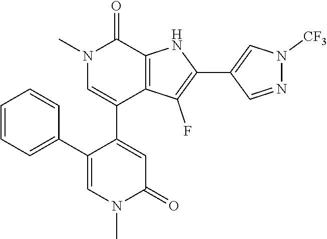 [SMILES: Cn1cc(-c2ccccc2)c(-c2cn(C)c(=O)c3[nH]c(-c4cnn(C(F)(F)F)c4)c(F)c23)cc1=O]

### Preparation 98. 3-fluoro-6-methyl-4-(1-methyl-2-oxo-5-phenyl-1,2-dihydropyridin-4-yl)-2-(1-(trifluoromethyl)-1H-pyrazol-4-yl)-1,6-dihydro-7H-pyrrolo[2,3-c]pyridin-7-one

To a stirred solution of 6-methyl-4-(1-methyl-2-oxo-5-phenyl-1,2-dihydropyridin-4-yl)-2-(1-(trifluoromethyl)-1H-pyrazol-4-yl)-1,6-dihydro-7H-pyrrolo[2,3-c]pyridin-7-one (500 mg, 1.07 mmol, 1.0 equiv) in DCM (10 mL) were added 1-fluoro-2,6-dichloropyridinium tetrafluoroborate (409 mg, 1.61 mmol, 1.5 equiv) at room temperature. The resulting mixture was stirred overnight at room temperature. The resulting mixture was concentrated under reduced pressure. The residue was purified by Prep-HPLC^{AG} and further purified CHIRAL-HPLC^{AH} to afford the title compound as a white solid (58.4 mg, 11%). ¹H NMR (400 MHz, DMSO-d⁶) δ 12.39 (s, 1H), 8.74 (s, 1H), 8.29 (s, 1H), 7.83 (s, 1H), 7.21 (s, 1H), 7.19-7.14 (m, 4H), 7.14-7.06 (m, 1H), 6.50 (s, 1H), 3.55 (s, 3H), 3.49 (s, 3H). LCMS^{P}: m/z=484 [M+H]⁺.

### Example 22: 4-(1-(2-(difluoromethoxy)ethyl)-2-oxo-5-phenyl-1,2-dihydropyridin-4-yl)-6-methyl-2-(1-(trifluoromethyl)-1H-pyrazol-4-yl)-1,6-dihydro-7H-pyrrolo[2,3-c]pyridin-7-one

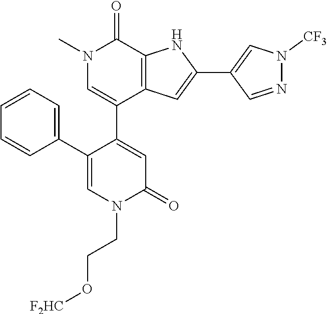 [SMILES: Cn1cc(-c2cc(=O)n(CCOC(F)F)cc2-c2ccccc2)c2cc(-c3cnn(C(F)(F)F)c3)[nH]c2c1=O]

### Preparation 99: 4-bromo-1-(2-hydroxyethyl)-5-iodopyridin-2 (1H)-one

To a stirred mixture of 4-bromo-5-iodopyridin-2 (1H)-one (15.0 g, 50.0 mmol, 1.00 equiv) and 2-bromoethan-1-ol (6.88 g, 55.0 mmol, 1.10 equiv) at room temperature in acetone (450 mL) was added K₂CO₃ (20.7 g, 150 mmol, 3.00 equiv). The resulting mixture at 60° C. was stirred overnight. The resulting mixture was filtered, the filter cake was washed with EtOAc (3×100 mL). The filtrate was concentrated under reduced pressure. The residue was purified by silica gel column chromatography, eluted with PE/EA (1:2) to afford the title compound as a yellow solid (9.00 g, 52.3%). LCMS: m/z=346 [M+H]⁺

### Preparation 100: 4-bromo-1-(2-hydroxyethyl)-5-iodopyridin-2 (1H)-one

To a solution of 4-bromo-1-(2-hydroxyethyl)-5-iodopyridin-2 (1H)-one (4.0 g, 11.6 mmol, 1.00 equiv) in DCM (50 mL) and H₂O (50 mL) at 0° C. was added KOAc (4.71 g, 48.0 mmol, 6.00 equiv). The mixture was stirred for 5 min and followed by the addition of (bromodifluoromethyl) trimethylsilane (10.4 g, 51.2 mmol, 4.40 equiv). The resulting mixture at room temperature was stirred overnight. The reaction at 0° C. was quenched with sat. NaHCO₃. The resulting mixture was extracted with EtOAc (5×20 mL). The combined organic layers were washed with brine, dried over anhydrous Na₂SO₄. After filtration, the filtrate was concentrated under reduced pressure. The residue was purified by silica gel column chromatography, eluted with PE/EA (3:7) to afford the title compound as a light-yellow solid (750 mg, 16.4%). LCMS: m/z=396 [M+H]⁺

### Preparation 101: 4-bromo-1-(2-(difluoromethoxy)ethyl)-5-phenylpyridin-2 (1H)-one

To a stirred mixture of 4-bromo-1-(2-(difluoromethoxy)ethyl)-5-iodopyridin-2 (1H)-one (700 mg, 1.78 mmol, 1.00 equiv) and phenyl boronic acid (325 mg, 2.67 mmol, 1.50 equiv) in EtOH (2.0 mL) and H₂O (2.0 mL) at room temperature was added Pd(PPh₃)₄ (205 mg, 0.178 mmol, 0.100 equiv) and Na₂CO₃ (377 mg, 3.55 mmol, 2.00 equiv). The resulting mixture at 80° C. under nitrogen atmosphere was stirred for 3 h. The reaction at room temperature was quenched with water. The resulting mixture was extracted with EtOAc (3×10 mL). The combined organic layers were washed with brine, dried over anhydrous Na₂SO₄. After filtration, the filtrate was concentrated under reduced pressure. The residue was purified by silica gel column chromatography, eluted with PE/EA (1:1) to afford the title compound as a yellow oil (330 mg, 54.0%). LCMS: m/z=346 [M+H]⁺

### Preparation 102: 2-chloro-4-(1-(2-(difluoromethoxy)ethyl)-2-oxo-5-phenyl-1,2-dihydropyridin-4-yl)-6-methyl-1-tosyl-1,6-dihydro-7H-pyrrolo[2, 3-c]pyridin-7-one

To a stirred mixture of 4-bromo-1-(2-(difluoromethoxy)ethyl)-5-iodopyridin-2 (1H)-one (300 mg, 0.872 mmol, 1.00 equiv) and 2-chloro-4-(5,5-dimethyl-1,3,2-dioxaborinan-2-yl)-6-methyl-1-tosyl-1,6-dihydro-7H-pyrrolo[2,3-c]pyridin-7-one (587 mg, 1.31 mmol, 1.50 equiv) in dioxane (5.0 mL) and H₂O (1.0 mL) at room temperature was added Pd(dppf)Cl₂·CH₂Cl₂ (71.0 mg, 0.087 mmol, 0.100 equiv) and Na₂CO₃ (185 mg, 1.74 mmol, 2.00 equiv). The resulting mixture at 60° C. under nitrogen atmosphere was stirred overnight. The reaction was quenched with water at room temperature. The resulting mixture was extracted with EtOAc (3×10 mL). The combined organic layers were washed with brine, dried over anhydrous Na₂SO₄. After filtration, the filtrate was concentrated under reduced pressure. The residue was purified by silica gel column chromatography, eluted with EtOAc/MeOH (9:1) to the title product as a light-yellow solid (167 mg, 33.9%). LCMS: m/z=600 [M+H]⁺.

### Preparation 103: 4-(1-(2-(difluoromethoxy)ethyl)-2-oxo-5-phenyl-1,2-dihydropyridin-4-yl)-6-methyl-1-tosyl-2-(1-(trifluoromethyl)-1H-pyrazol-4-yl)-1,6-dihydro-7H-pyrrolo[2,3-c]pyridin-7-one

Following the procedure described in preparation 8, 2-chloro-4-(1-(2-(difluoromethoxy)ethyl)-2-oxo-5-phenyl-1,2-dihydropyridin-4-yl)-6-methyl-1-tosyl-1,6-dihydro-7H-pyrrolo[2,3-c]pyridin-7-one (150 mg, 0.250 mmol, 1.00 equiv) and 4-(4,4,5,5-tetramethyl-1,3,2-dioxaborolan-2-yl)-1-(trifluoromethyl)-1H-pyrazole (131 mg, 0.500 mmol, 2.00 equiv) was reacted to give title compound as a white solid (160 mg, 91.48%). LCMS: m/z=700 [M+H]⁺.

### Preparation 104: 4-(1-(2-(difluoromethoxy)ethyl)-2-oxo-5-phenyl-1,2-dihydropyridin-4-yl)-6-methyl-2-(1-(trifluoromethyl)-1H-pyrazol-4-yl)-1,6-dihydro-7H-pyrrolo[2, 3-c]pyridin-7-one

Following the procedure described in preparation 7, 2-chloro-4-(1-(2-(difluoromethoxy)ethyl)-2-oxo-5-phenyl-1,2-dihydropyridin-4-yl)-6-methyl-1-tosyl-1,6-dihydro-7H-pyrrolo[2,3-c]pyridin-7-one (150 mg, 0.214 mmol, 1.00 equiv) was reacted to afford the title compound as a white solid (85.4 mg, 73.0%) after purification by Prep-HPLC^{AI 1}H NMR (400 MHz, DMSO-d⁶) δ 12.30 (s, 1H), 8.91 (s, 1H), 8.43 (s, 1H), 7.80 (s, 1H), 7.20-7.13 (m, 5H), 7.08 (s, 1H), 6.74 (s, 1H), 6.55-6.54 (m, 1H), 6.33 (s, 1H), 4.30-4.29 (m, 2H), 4.21-4.19 (m, 2H), 3.42 (s, 3H). LCMSc: m/z=546 [M+H]⁺.

### Example 23 (Compound C): 6-methyl-4-(1-(2-(methylthio)ethyl)-2-oxo-5-phenyl-1,2-dihydropyridin-4-yl)-2-(1-(trifluoromethyl)-1H-pyrazol-4-yl)-1,6-dihydro-7H-pyrrolo[2,3-c]pyridin-7-one

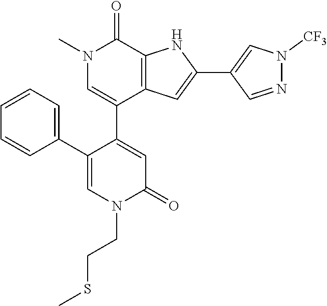 [SMILES: CSCCn1cc(-c2ccccc2)c(-c2cn(C)c(=O)c3[nH]c(-c4cnn(C(F)(F)F)c4)cc23)cc1=O]

### Preparation 105: 4-bromo-5-iodo-1-(2-(methylthio)ethyl)pyridin-2 (1H)-one

To a stirred mixture of 4-bromo-5-iodopyridin-2 (1H)-one (5.00 g, 16.7 mmol, 1.00 equiv) and K₂CO₃ (4.61 g, 33.3 mmol, 2.00 equiv) in DMF (100 mL) at room temperature was added (2-chloroethyl)(methyl)sulfane (2.03 g, 18.3 mmol, 1.10 equiv). The resulting mixture at 100° C. was stirred for 2 h. The reaction at room temperature was quenched with water. The resulting mixture was extracted with EtOAc (3×80 mL). The combined organic layers were washed with brine, dried over anhydrous Na₂SO₄. After filtration, the filtrate was concentrated under reduced pressure. The residue was purified by RP-HPLC^{AJ} to afford the title compound as a yellow solid (2.08 g, 33.3%). LCMS: m/z=376 [M+H]⁺

### Preparation 106: 4-bromo-1-(2-(methylthio)ethyl)-5-phenylpyridin-2 (1H)-one

To a stirred solution of 4-bromo-5-iodo-1-(2-(methylthio)ethyl)pyridin-2 (1H)-one (1.92 g, 5.12 mmol, 1.00 equiv) and phenyl boronic acid (937 mg, 7.68 mmol, 1.50 equiv) in DMF (30 mL) at room temperature was added Na₂CO₃ (1.09 g, 10.3 mmol, 2.01 equiv) and Pd(PPh₃)₄ (592 mg, 0.512 mmol, 0.100 equiv). The resulting mixture at 100° C. under nitrogen atmosphere was stirred for 5 h. The resulting mixture was extracted with EtOAc (3×100 mL). The combined organic layers were washed with brine, dried over anhydrous Na₂SO₄. After filtration, the filtrate was concentrated under reduced pressure. The crude product (2.50 g) was purified by RP-HPLC^{AK} to afford the title compound as a yellow liquid (946 mg, 57.0%). LCMS: m/z=326 [M+H]⁺.

### Preparation 107: 2-chloro-6-methyl-4-(1-(2-(methylthio)ethyl)-2-oxo-5-phenyl-1,2-dihydropyridin-4-yl)-1-tosyl-1,6-dihydro-7H-pyrrolo[2,3-c]pyridin-7-one

To a stirred mixture of 4-bromo-1-(2-(methylthio)ethyl)-5-phenylpyridin-2 (1H)-one (630 mg, 1.94 mmol, 1.00 equiv) and 2-chloro-4-(5,5-dimethyl-1,3,2-dioxaborinan-2-yl)-6-methyl-1-tosyl-1,6-dihydro-7H-pyrrolo[2,3-c]pyridin-7-one (872 mg, 1.94 mmol, 1.00 equiv) in DME (15.0 mL) and H₂O (3.0 mL) at room temperature was added Pd(dppf)Cl₂·CH₂Cl₂ (158 mg, 0.19 mmol, 0.100 equiv) and Na₂CO₃ (411 mg, 3.88 mmol, 2.00 equiv). The resulting mixture at 60° C. under nitrogen atmosphere was stirred for overnight. The resulting mixture was extracted with EtOAc (3×15 mL). The combined organic layers were washed with brine, dried over anhydrous Na₂SO₄. After filtration, the filtrate was concentrated under reduced pressure. The residue was purified by silica gel column chromatography, eluted with MeOH/EA (1:10) to afford the title compound as a yellow solid (803 mg, 71.2%). LCMS: m/z=580 [M+H]⁺.

### Preparation 108: 6-methyl-4-(1-(2-(methylthio)ethyl)-2-oxo-5-phenyl-1,2-dihydropyridin-4-yl)-1-tosyl-2-(1-(trifluoromethyl)-1H-pyrazol-4-yl)-1,6-dihydro-7H-pyrrolo[2, 3-c]pyridin-7-one

Following the procedure in preparation 8, 2-chloro-6-methyl-4-(1-(2-(methylthio)ethyl)-2-oxo-5-phenyl-1,2-dihydropyridin-4-yl)-1-tosyl-1,6-dihydro-7H-pyrrolo[2,3-c]pyridin-7-one (600 mg, 1.03 mmol, 1.00 equiv) and 4-(4,4,5,5-tetramethyl-1,3,2-dioxaborolan-2-yl)-1-(trifluoromethyl)-1H-pyrazole (406 mg, 1.55 mmol, 1.50 equiv) was reacted to afford the title compound as a yellow solid (470 mg, 66.9%). LCMS: m/z=680 [M+H]⁺.

### Preparation 109: 6-methyl-4-(1-(2-(methylthio)ethyl)-2-oxo-5-phenyl-1,2-dihydropyridin-4-yl)-2-(1-(trifluoromethyl)-1H-pyrazol-4-yl)-1,6-dihydro-7H-pyrrolo[2,3-c]pyridin-7-one

Following the procedure in preparation 7, 6-methyl-4-(1-(2-(methylthio)ethyl)-2-oxo-5-phenyl-1,2-dihydropyridin-4-yl)-1-tosyl-2-(1-(trifluoromethyl)-1H-pyrazol-4-yl)-1,6-dihydro-7H-pyrrolo[2,3-c]pyridin-7-one (470 mg, 0.691 mmol, 1.0 equiv) was reacted to afford the title compound as a white solid (168.8 mg, 45.9%) after purification by RP-HPLC^{AL 1}H NMR (400 MHz, DMSO-d⁶) δ 12.31 (s, 1H), 8.91 (s, 1H), 8.43 (s, 1H), 7.89 (s, 1H), 7.21-7.19 (m, 4H), 7.16-7.10 (m, 1H), 7.08 (s, 1H), 6.50 (s, 1H), 6.33 (s, 1H), 4.20 (t, J=6.9 Hz, 2H), 3.42 (s, 3H), 2.88 (t, J=6.9 Hz, 2H), 2.15 (s, 3H). LCMS^{R}: m/z=526 [M+H]⁺.

### Example 24: 6-methyl-4-(1-methyl-5-(4-((methylthio)methyl)phenyl)-2-oxo-1,2-dihydropyridin-4-yl)-2-(1-(trifluoromethyl)-1H-pyrazol-4-yl)-1,6-dihydro-7H-pyrrolo[2,3-c]pyridin-7-one

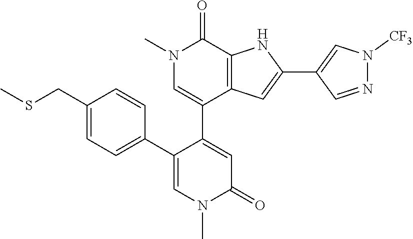 [SMILES: CSCc1ccc(-c2cn(C)c(=O)cc2-c2cn(C)c(=O)c3[nH]c(-c4cnn(C(F)(F)F)c4)cc23)cc1]

### Preparation 110: 4,4,5,5-tetramethyl-2-(4-((methylthio)methyl)phenyl)-1,3,2-dioxaborolane

To a stirred solution of 2-(4-(bromomethyl)phenyl)-4,4,5,5-tetramethyl-1,3,2-dioxaborolane (6.00 g, 20.2 mmol, 1.00 equiv) in DMF (50.0 mL) at room temperature was added sodium methanethiolate (141 mg, 2.00 mmol, 1.20 equiv). The resulting mixture at room temperature was stirred overnight. The reaction at room temperature was quenched with water. The resulting mixture was extracted with EtOAc (3×50 mL). The combined organic layers were washed with brine, dried over anhydrous Na₂SO₄. After filtration, the filtrate was concentrated under reduced pressure. The residue was purified by RP-HPLC^{AM} to afford the title compound as a light-yellow solid (3.70 g, 69.3%). ¹H NMR (400 MHz, DMSO-d⁶) δ 7.66 (d, J=7.8 Hz, 2H), 7.33 (d, J=7.8 Hz, 2H), 3.71 (s, 2H), 1.94 (s, 3H), 1.31 (s, 12H).

### Preparation 111: 4-bromo-1-methyl-5-{4-[(methylsulfanyl)methyl]phenyl}pyridin-2-one

To a stirred mixture of 4,4,5,5-tetramethyl-2-(4-((methylthio)methyl)phenyl)-1,3,2-dioxaborolane (3.48 g, 13.1 mmol, 1.20 equiv) and 4-bromo-5-iodo-1-methylpyridin-2-one (3.44 g, 10.9 mmol, 1.00 equiv) in DMF (15.0 mL) and H₂O (1.50 mL) was added Pd(PPh₃)₄ (0.630 g, 0.549 mmol, 0.05 equiv) and Na₂CO₃ (2.33 g, 21.9 mmol, 2.00 equiv). The resulting mixture at 100° C. under nitrogen atmosphere was stirred overnight. The resulting mixture was extracted with EtOAc (3×100 mL). The combined organic layers were washed with brine, dried over anhydrous Na₂SO₄. After filtration, the filtrate was concentrated under reduced pressure. The crude product (4.00 g) was purified by Prep-HPLC^{AN} to afford the title compound as a yellow solid (1.60 g, 42.6%). LCMS: m/z=326 [M+H]⁺.

### Preparation 112: 2-chloro-6-methyl-4-(1-methyl-5-(4-((methylthio)methyl)phenyl)-2-oxo-1,2-dihydropyridin-4-yl)-1-tosyl-1,6-dihydro-7H-pyrrolo[2, 3-c]pyridin-7-one

To a stirred mixture of 4-bromo-1-methyl-5-{4-[(methylsulfanyl)methyl]phenyl}pyridin-2-one (802 mg, 2.47 mmol, 1.11 equiv) and 2-chloro-4-(5,5-dimethyl-1,3,2-dioxaborinan-2-yl)-6-methyl-1-tosyl-1,6-dihydro-7H-pyrrolo[2,3-c]pyridin-7-one (1.00 g, 2.22 mmol, 1.00 equiv) in DME (20.0 mL) and H₂O (4.0 mL) at room temperature was added Na₂CO₃ (472 mg, 4.45 mmol, 2.00 equiv) and Pd(dppf)Cl₂·CH₂Cl₂ (181 mg, 0.223 mmol, 0.10 equiv). The resulting mixture at 60° C. under nitrogen atmosphere was stirred overnight. The resulting mixture was extracted with EtOAc (3×20 mL). The combined organic layers were washed with brine, dried over anhydrous Na₂SO₄. After filtration, the filtrate was concentrated under reduced pressure. The residue was purified by silica gel column chromatography, eluted with MeOH/EA (1:10) to afford the title compound as a yellow solid (1.05 g, 81.2%). LCMS: m/z=580 [M+H]⁺.

### Preparation 113: 6-methyl-4-(1-methyl-5-(4-((methylthio)methyl)phenyl)-2-oxo-1,2-dihydropyridin-4-yl)-1-tosyl-2-(1-(trifluoromethyl)-1H-pyrazol-4-yl)-1,6-dihydro-7H-pyrrolo[2,3-c]pyridin-7-one

Following the procedure in preparation 8, 2-chloro-6-methyl-4-(1-methyl-5-(4-((methylthio)methyl)phenyl)-2-oxo-1,2-dihydropyridin-4-yl)-1-tosyl-1,6-dihydro-7H-pyrrolo[2,3-c]pyridin-7-one (700 mg, 1.20 mmol, 1.00 equiv) and 4-(4,4,5,5-tetramethyl-1,3,2-dioxaborolan-2-yl)-1-(trifluoromethyl)-1H-pyrazole (474 mg, 1.81 mmol, 1.50 equiv) was reacted to afford the title compound as a yellow solid (630 mg, 76.8%). LCMS: m/z=680 [M+H]⁺

### Preparation 114: 6-methyl-4-(1-methyl-5-(4-((methylthio)methyl)phenyl)-2-oxo-1,2-dihydropyridin-4-yl)-2-(1-(trifluoromethyl)-1H-pyrazol-4-yl)-1,6-dihydro-7H-pyrrolo[2,3-c]pyridin-7-one

To a stirred mixture of 6-methyl-4-(1-methyl-5-(4-((methylthio)methyl)phenyl)-2-oxo-1,2-dihydropyridin-4-yl)-1-tosyl-2-(1-(trifluoromethyl)-1H-pyrazol-4-yl)-1,6-dihydro-7H-pyrrolo[2,3-c]pyridin-7-one (630 mg, 0.927 mmol, 1.00 equiv) in MeOH (9.00 mL) and H₂O (1.80 mL) at room temperature was added NaOH (370 mg, 9.27 mmol, 10.0 equiv). The resulting mixture was stirred for 1 h at 60° C. The resulting mixture was extracted with EtOAc (3×10 mL). The combined organic layers were washed with brine, dried over anhydrous Na₂SO₄. After filtration, the filtrate was concentrated under reduced pressure. The crude product (510 mg) was purified by Prep-HPLC^{AO} to afford the title compound as a white solid (279 mg, 56.7%). ¹H NMR (400 MHz, DMSO-d⁶) δ 12.28 (s, 1H), 8.86 (s, 1H), 8.40 (s, 1H), 7.89 (s, 1H), 7.13-7.08 (m, 5H), 6.49 (s, 1H), 6.28 (s, 1H), 3.55-3.53 (m, 5H), 3.43 (s, 3H), 1.63 (s, 3H). LCMS^{R}: m/z=526 [M+H]⁺

### Example 25: 6-methyl-4-(1-methyl-5-(3-((methylthio)methyl)phenyl)-2-oxo-1,2-dihydropyridin-4-yl)-2-(1-(trifluoromethyl)-1H-pyrazol-4-yl)-1,6-dihydro-7H-pyrrolo[2,3-c]pyridin-7-one

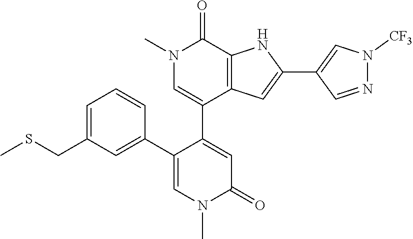 [SMILES: CSCc1cccc(-c2cn(C)c(=O)cc2-c2cn(C)c(=O)c3[nH]c(-c4cnn(C(F)(F)F)c4)cc23)c1]

### Preparation 115: 4,4,5,5-tetramethyl-2-(3-((methylthio)methyl)phenyl)-1,3,2-dioxaborolane

To a stirred solution of 2-(3-(bromomethyl)phenyl)-4,4,5,5-tetramethyl-1,3,2-dioxaborolane (10.0 g, 33.6 mmol, 1.00 equiv) in DMF (100 mL) at room temperature was added (methylsulfanyl)sodium (2.83 g, 40.4 mmol, 1.20 equiv). The resulting mixture at room temperature was stirred overnight. The resulting mixture was extracted with EtOAc (3×200 mL). The combined organic layers were washed with brine, dried over anhydrous Na₂SO₄. After filtration, the filtrate was concentrated under reduced pressure. The residue was purified by silica gel column chromatography, eluted with PE/EA (1:1) to afford the title compound as a colorless oil (6.00 g, 60.7%). ¹H NMR (400 MHz, DMSO-d⁶) δ 7.63 (t, J=1.5 Hz, 1H), 7.57-7.49 (m, 1H), 7.45-7.38 (m, 1H), 7.33 (t, J=7.4 Hz, 1H), 3.70 (s, 2H), 1.93 (s, 3H), 1.30 (s, 12H).

### Preparation 116: 4-bromo-1-methyl-5-(3-((methylthio)methyl)phenyl)pyridin-2 (1H)-one

To a stirred mixture of 4,4,5,5-tetramethyl-2-(3-((methylthio)methyl)phenyl)-1,3,2-dioxaborolane (3.03 g, 11.4 mmol, 1.20 equiv) and 4-bromo-5-iodo-1-methylpyridin-2 (1H)-one (3.00 g, 9.55 mmol, 1.00 equiv) in DMF (15.0 mL) and H₂O (1.5 mL) at room temperature was added Na₂CO₃ (2.03 g, 19.1 mmol, 2.00 equiv) and Pd(PPh₃)₄ (1.10 g, 0.956 mmol, 0.100 equiv). The resulting mixture at 100° C. under nitrogen atmosphere was stirred for 5 h. The resulting mixture was extracted with EtOAc (3×20.0 mL). The combined organic layers were washed with brine, dried over anhydrous Na₂SO₄. After filtration, the filtrate was concentrated under reduced pressure. The residue was purified by silica gel column chromatography, eluted with PE/EA (1:1) to afford the title compound as a brown oil (1.80 g, 52.3%). LCMS: m/z=326 [M+H]⁺

### Preparation 117: 2-chloro-6-methyl-4-(1-methyl-5-(3-((methylthio)methyl)phenyl)-2-oxo-1,2-dihydropyridin-4-yl)-1-tosyl-1,6-dihydro-7H-pyrrolo[2, 3-c]pyridin-7-one

To a stirred mixture of 4-bromo-1-methyl-5-(3-((methylthio)methyl)phenyl)pyridin-2 (1H)-one (0.790 g, 2.45 mmol, 1.10 equiv) and 2-chloro-4-(5,5-dimethyl-1,3,2-dioxaborinan-2-yl)-6-methyl-1-tosyl-1,6-dihydro-7H-pyrrolo[2,3-c]pyridin-7-one (1.00 g, 2.23 mmol, 1.00 equiv) in DME (20.0 mL) and H₂O (4.0 mL) at room temperature were added Na₂CO₃ (0.470 g, 4.46 mmol, 2.00 equiv) and Pd(dppf)Cl₂·CH₂Cl₂ (0.180 g, 0.223 mmol, 0.100 equiv). The resulting mixture at 60° C. under nitrogen atmosphere was stirred for 1 h. The resulting mixture was extracted with EtOAc (3×30.0 mL). The combined organic layers were washed with brine, dried over anhydrous Na₂SO₄. After filtration, the filtrate was concentrated under reduced pressure. The residue was purified by silica gel column chromatography, eluted with EA/MeOH (10:1) to afford the title compound as a brown solid (1.17 g, 76.9%). LCMS: m/z=580 [M+H]⁺.

### Preparation 118: 6-methyl-4-(1-methyl-5-(3-((methylthio)methyl)phenyl)-2-oxo-1,2-dihydropyridin-4-yl)-1-tosyl-2-(1-(trifluoromethyl)-1H-pyrazol-4-yl)-1,6-dihydro-7H-pyrrolo[2,3-c]pyridin-7-one

Following the procedure in preparation 8, 2-chloro-6-methyl-4-(1-methyl-5-(3-((methylthio)methyl)phenyl)-2-oxo-1,2-dihydropyridin-4-yl)-1-tosyl-1,6-dihydro-7H-pyrrolo[2,3-c]pyridin-7-one (250 mg, 0.431 mmol, 1.00 equiv) and 4-(4,4,5,5-tetramethyl-1,3,2-dioxaborolan-2-yl)-1-(trifluoromethyl)-1H-pyrazole (225 mg, 0.862 mmol, 2.00 equiv) was reacted to afford the title compound as a light-yellow solid (460 mg, 78.6%). LCMS: m/z=680 [M+H]⁺

### Preparation 119: 6-methyl-4-(1-methyl-5-(3-((methylthio)methyl)phenyl)-2-oxo-1,2-dihydropyridin-4-yl)-2-(1-(trifluoromethyl)-1H-pyrazol-4-yl)-1,6-dihydro-7H-pyrrolo[2,3-c]pyridin-7-one

To a stirred solution of 6-methyl-4-(1-methyl-5-(3-((methylthio)methyl)phenyl)-2-oxo-1,2-dihydropyridin-4-yl)-1-tosyl-2-(1-(trifluoromethyl)-1H-pyrazol-4-yl)-1,6-dihydro-7H-pyrrolo[2,3-c]pyridin-7-one (440 mg, 0.647 mmol, 1.00 equiv) in MeOH (5.0 mL) and H₂O (1.0 mL) at room temperature was added NaOH (258 mg, 6.47 mmol, 10.0 equiv). The resulting mixture was stirred for 1 h at 60° C. The resulting mixture was extracted with EtOAc (3×6.0 mL). The combined organic layers were washed with brine, dried over anhydrous Na₂SO₄. After filtration, the filtrate was concentrated under reduced pressure. The crude product (400 mg) was purified by Prep-HPLC^{AP} to afford the title compound as a white solid. ¹H NMR (400 MHz, DMSO-d₆) δ 12.26 (s, 1H), 8.87 (s, 1H), 8.41 (s, 1H), 7.87 (s, 1H), 7.20-7.07 (m, 4H), 7.05-6.97 (m, 1H), 6.49 (s, 1H), 6.28 (s, 1H), 3.56 (s, 3H), 3.52-3.39 (m, 5H), 1.50 (s, 3H). LCMST: m/z=526 [M+H]⁺

### Example 26: 2-(2,6-dimethylpyridin-4-yl)-4-(5-(2-fluorophenyl)-1-methyl-2-oxo-1,2-dihydropyridin-4-yl)-6-methyl-1,6-dihydro-7H-pyrrolo[2,3-c]pyridin-7-one hydrogen chloride

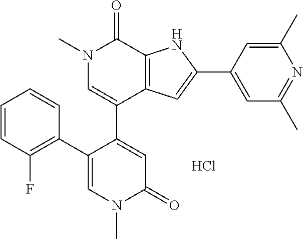 [SMILES: Cc1cc(-c2cc3c(-c4cc(=O)n(C)cc4-c4ccccc4F)cn(C)c(=O)c3[nH]2)cc(C)n1 ; Cl]

### Preparation 120: 4-bromo-2-(2,6-dimethylpyridin-4-yl)-6-methyl-1-tosyl-1,6-dihydro-7H-pyrrolo[2,3-c]pyridin-7-one

To a stirred mixture of 4-bromo-2-iodo-6-methyl-1-tosyl-1,6-dihydro-7H-pyrrolo[2,3-c]pyridin-7-one (14.5 g, 28.6 mmol, 1.00 equiv) and (2,6-dimethylpyridin-4-yl)boronic acid (21.6 g, 143 mmol, 5.00 equiv) in 1,4-dioxane (200 mL) and H₂O (50 mL) at room temperature were added K₃PO₄ (12.1 g, 57.2 mmol, 2.00 equiv) and Pd₂(dba)₃ (2.62 g, 2.86 mmol, 0.100 equiv). The resulting mixture at 50° C. under nitrogen atmosphere was stirred overnight. The resulting mixture was concentrated under reduced pressure. The resulting mixture was extracted with EtOAc (3×80 mL). The combined organic layers were washed with brine, dried over anhydrous Na₂SO₄. After filtration, the filtrate was concentrated under reduced pressure. The residue was purified by silica gel column chromatography, eluted with PE/EA (1:1) to afford the title compound as a white solid (5.00 g, 36.0%). LCMS: m/z=488 [M+H]⁺

### Preparation 121: (2-(2,6-dimethylpyridin-4-yl)-6-methyl-7-oxo-1-tosyl-6,7-dihydro-1H-pyrrolo[2,3-c]pyridin-4-yl)boronic acid

To a mixture of 4-bromo-2-(2,6-dimethylpyridin-4-yl)-6-methyl-1-(4-methylbenzenesulfonyl)pyrrolo[2,3-c]pyridin-7-one (5.00 g, 10.3 mmol, 1.00 equiv) and 5,5,5′,5′-tetramethyl-2,2′-bi(1,3,2-dioxaborinane) (14.0 g, 61.8 mmol, 6.00 equiv) in dioxane (150 mL) at room temperature was added Pd(dppf)Cl₂·CH₂Cl₂ (841 mg, 1.03 mmol, 0.100 equiv) and KOAc (4.04 g, 41.2 mmol, 4.00 equiv). The resulting mixture at 60° C. under nitrogen atmosphere was stirred overnight. The reaction at room temperature was quenched with water. The resulting mixture was extracted with EtOAc (4×150 mL). The combined organic layers were washed with brine, dried over anhydrous Na₂SO₄. After filtration, the filtrate was concentrated under reduced pressure. The residue was purified by silica gel column chromatography, eluted with PE/EA (5:1) to afford the crude compound as a yellow solid (14.0 g, crude). The crude compound was further purified by RP-HPLC^{AQ} to afford the title compound as a yellow solid (4.20 g, 90.1%). LCMS: m/z=452 [M+H]⁺

### Preparation 122: 4-bromo-5-(2-fluorophenyl)-1-methylpyridin-2 (1H)-one

To a stirred mixture of 4-bromo-5-iodo-1-methylpyridin-2 (1H)-one (5.00 g, 15.9 mmol, 1.00 equiv) and (2-fluorophenyl)boronic acid (3.12 g, 22.3 mmol, 1.40 equiv) in DME (30.0 mL) and H₂O (3.00 mL) at room temperature was added Na₂CO₃ (3.38 g, 31.8 mmol, 2.00 equiv) and Pd(PPh₃)₄ (920 mg, 0.796 mmol, 0.050 equiv). The resulting mixture at 120° C. under nitrogen atmosphere was stirred for 6 h. The resulting mixture was extracted with EtOAc (3×150 mL). The combined organic layers were washed with brine, dried over anhydrous Na₂SO₄. After filtration, the filtrate was concentrated under reduced pressure. The residue was purified by RP-HPLC^{AR} to afford the title compound as a brown solid (3.00 g, 60.7%). LCMS: m/z=284 [M+H]⁺

### Preparation 123: 2-(2,6-dimethylpyridin-4-yl)-4-(5-(2-fluorophenyl)-1-methyl-2-oxo-1,2-dihydropyridin-4-yl)-6-methyl-1-tosyl-1,6-dihydro-7H-pyrrolo[2,3-c]pyridin-7-one

To a mixture of (2-(2,6-dimethylpyridin-4-yl)-6-methyl-7-oxo-1-tosyl-6,7-dihydro-1H-pyrrolo[2,3-c]pyridin-4-yl)boronic acid (301 mg, 0.670 mmol, 1.00 equiv) and 4-bromo-5-(2-fluorophenyl)-1-methylpyridin-2 (1H)-one (285 mg, 1.00 mmol, 1.50 equiv) in DME (15.0 mL) and H₂O (3.0 mL) at room temperature was added Pd(dppf)Cl₂·CH₂Cl₂ (48.8 mg, 0.067 mmol, 0.100 equiv) and Na₂CO₃ (141 mg, 1.33 mmol, 2.00 equiv). The resulting mixture at 60° C. under nitrogen atmosphere was stirred for 2 h. The reaction was quenched with water. The resulting mixture was extracted with EtOAc (3×20 mL). The combined organic layers were washed with brine, dried over anhydrous Na₂SO₄. After filtration, the filtrate was concentrated under reduced pressure. The residue was purified by silica gel column chromatography, eluted with EA/MeOH (10:1) to afford the title compound as a yellow solid (248 mg, 61.1%). LCMS: m/z=609 [M+H]⁺

### Preparation 124: 4-[2-(2,6-dimethylpyridin-4-yl)-6-methyl-7-oxo-1H-pyrrolo[2,3-c]pyridin-4-yl]-5-(2-fluorophenyl)-1-methylpyridin-2-one hydrochloride

To a mixture of 2-(2,6-dimethylpyridin-4-yl)-4-(5-(2-fluorophenyl)-1-methyl-2-oxo-1,2-dihydropyridin-4-yl)-6-methyl-1-tosyl-1,6-dihydro-7H-pyrrolo[2,3-c]pyridin-7-one (240 mg, 0.394 mmol, 1.00 equiv) in MeOH (4.0 mL) and H₂O (1.0 mL) at room temperature was added NaOH (157 mg, 3.94 mmol, 10.0 equiv). The resulting mixture was stirred at 60° C. for 1 h. The reaction at room temperature was quenched with water. The resulting mixture was extracted with EtOAc (5×10 mL). The combined organic layers were washed with brine, dried over anhydrous Na₂SO₄. After filtration, the filtrate was concentrated under reduced pressure. The crude product was purified by Prep-HPLC^{AS} to afford the product. The product was treated it with HCl in MeOH (2.0 mL, 4.0 M/L) and lyophilized to afford the title compound as a yellow solid (108 mg, 52.4%). ¹H NMR (400 MHz, DMSO-d⁶) δ 15.22 (br, 1H), 12.98 (s, 1H), 8.24 (s, 2H), 7.96 (s, 1H), 7.48-7.38 (m, 1H), 7.26-7.19 (m, 1H), 7.17-7.09 (m, 2H), 7.04-6.93 (m, 2H), 6.56 (s, 1H), 3.56 (s, 3H), 3.38 (s, 3H), 2.67 (s, 6H). LCMS^{U}: m/z=455 [M+H]⁺.

### Example 27: 2-(2,6-dimethylpyridin-4-yl)-6-methyl-4-(1-methyl-2-oxo-5-(o-tolyl)-1,2-dihydropyridin-4-yl)-1,6-dihydro-7H-pyrrolo[2,3-c]pyridin-7-one hydrochloride (BA59)

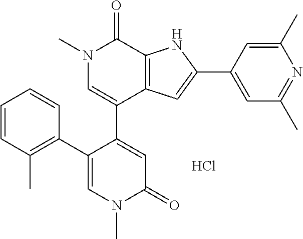 [SMILES: Cc1cc(-c2cc3c(-c4cc(=O)n(C)cc4-c4ccccc4C)cn(C)c(=O)c3[nH]2)cc(C)n1 ; Cl]

### Preparation 125: 4-bromo-1-methyl-5-(o-tolyl)pyridin-2 (1H)-one

To a mixture of 4-bromo-5-iodo-1-methylpyridin-2 (1H)-one (5.00 g, 16.0 mmol, 1.00 equiv) and o-tolylboronic acid (3.26 g, 24.0 mmol, 2.00 equiv) in DMF (100 mL) and H₂O (10 mL) at room temperature was added Pd(PPh₃)₄ (1.85 g, 1.60 mmol, 0.100 equiv) and Na₂CO₃ (3.39 g, 32.0 mmol, 2.00 equiv). The resulting mixture at 100° C. under nitrogen atmosphere was stirred for 3 h. The reaction was quenched with water at room temperature. The resulting mixture was extracted with EtOAc (3×300 mL). The combined organic layers were washed with brine, dried over anhydrous Na₂SO₄. After filtration, the filtrate was concentrated under reduced pressure. The residue was purified by RP-HPLC^{AT} to afford the title compound as a yellow liquid (2.30 g, 52.0%). LCMS: m/z=280 [M+H]⁺

### Preparation 126: 2-(2,6-dimethylpyridin-4-yl)-6-methyl-4-(1-methyl-2-oxo-5-(o-tolyl)-1,2-dihydropyridin-4-yl)-1-tosyl-1,6-dihydro-7H-pyrrolo[2,3-c]pyridin-7-one

To a mixture of (2-(2,6-dimethylpyridin-4-yl)-6-methyl-7-oxo-1-tosyl-6,7-dihydro-1H-pyrrolo[2,3-c]pyridin-4-yl)boronic acid (250 mg, 0.554 mmol, 1.00 equiv) and 4-bromo-1-methyl-5-(o-tolyl)pyridin-2 (1H)-one (308 mg, 1.11 mmol, 2.00 equiv) in DME (10.0 mL) and H₂O (2.0 mL) at room temperature was added Pd(dppf)Cl₂·CH₂Cl₂ (40.5 mg, 0.055 mmol, 0.100 equiv) and Na₂CO₃ (117 mg, 1.11 mmol, 2.00 equiv). The resulting mixture at 60° C. under nitrogen atmosphere was stirred for 1 h. The reaction was quenched with water at room temperature. The resulting mixture was extracted with EtOAc (3×15 mL). The combined organic layers were washed with brine, dried over anhydrous Na₂SO₄. After filtration, the filtrate was concentrated under reduced pressure to afford the title compound as a yellow solid (586 mg, crude). The crude product was used in the next step directly without further purification. LCMS: m/z=605 [M+H]⁺

### Preparation 127: 2-(2,6-dimethylpyridin-4-yl)-6-methyl-4-(1-methyl-2-oxo-5-(o-tolyl)-1,2-dihydropyridin-4-yl)-1,6-dihydro-7H-pyrrolo[2,3-c]pyridin-7-one hydrochloride

To a mixture of 2-(2,6-dimethylpyridin-4-yl)-6-methyl-4-(1-methyl-2-oxo-5-(o-tolyl)-1,2-dihydropyridin-4-yl)-1-tosyl-1,6-dihydro-7H-pyrrolo[2,3-c]pyridin-7-one (420 mg, 0.695 mmol, 1.00 equiv) in MeOH (10.0 mL) and H₂O (2.5 mL) at room temperature was added NaOH (278 mg, 6.95 mmol, 10.0 equiv). The resulting mixture was stirred at 60° C. for 1 h. The reaction at room temperature was quenched with water. The resulting mixture was extracted with EtOAc (3×20 mL). The combined organic layers were washed with brine, dried over anhydrous Na₂SO₄. After filtration, the filtrate was concentrated under reduced pressure. The crude product was purified by Prep-HPLC^{AU} to afford the product, The product was further treated with HCl in MeOH (2.0 mL, 4.0 M/L) and lyophilized to afford the title compound as a yellow solid (106.7 mg, 32.7%). 1 NMR (400 MHz, Methanol-d₄) δ 8.10 (s, 2H), 7.75 (s, 1H), 739-7.27 (m, 2H), 7.25-7.17 (m, 2H), 7.14-7.05 (m, 1H), 6.87 (s, 1H), 6.72 (s, 1H), 3.71 (s, 3H), 3.35 (s, 3H), 2.77 (s, 6H), 1.94 (s, 3H). LCMS^{U}: m/z=451 [M+H]⁺.

### Example 28: 4-(1-cyclopropyl-2-oxo-5-phenyl-1,2-dihydropyridin-4-yl)-2-(2,6-dimethylpyridin-4-yl)-6-methyl-1,6-dihydro-7H-pyrrolo[2,3-c]pyridin-7-one hydrogen chloride

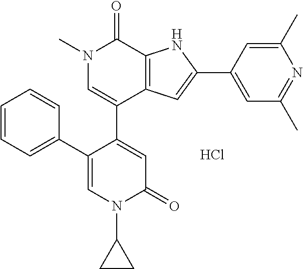 [SMILES: Cc1cc(-c2cc3c(-c4cc(=O)n(C5CC5)cc4-c4ccccc4)cn(C)c(=O)c3[nH]2)cc(C)n1 ; Cl]

### Preparation 128: 4-bromo-1-cyclopropyl-5-iodopyridin-2 (1H)-one

To a mixture of 4-bromo-5-iodo-1H-pyridin-2-one (1.00 g, 3.34 mmol, 1.00 equiv) and Na₂CO₃ (800 mg, 7.57 mmol, 2.27 equiv) in DCE (20.0 mL) at room temperature was stirred for 30 min. To the above mixture was added cyclopropylboronic acid (630 mg, 7.34 mmol, 2.20 equiv), Cu(OAc)₂ (650 mg, 3.57 mmol, 1.07 equiv), and 2,2′-bipyridine (560 mg, 3.57 mmol, 1.07 equiv). The resulting mixture at 60° C. under 02 atmosphere was stirred overnight. The resulting mixture was quenched with water. The resulting mixture was extracted with DCM (3×100 mL). The combined organic layers were dried over anhydrous Na₂SO₄. After filtration, the filtrate was concentrated under reduced pressure. The residue was purified by silica gel column chromatography, eluted with PE/EA (4:1) to afford the title compound as yellow solid (600 mg, 52.9%). LCMS: m/z=342 [M+H]⁺

### Preparation 129: 4-bromo-1-cyclopropyl-5-phenylpyridin-2 (1H)-one

To a stirred mixture of 4-bromo-1-cyclopropyl-5-iodopyridin-2 (1H)-one (600 mg, 1.77 mmol, 1.00 equiv) and phenyl boronic acid (430 mg, 3.53 mmol, 2.00 equiv) in DMF (10.0 mL) and H₂O (1.0 mL) at room temperature was added Pd(PPh₃)₄ (204 mg, 0.176 mmol, 0.100 equiv) and Na₂CO₃ (374 mg, 3.53 mmol, 2.00 equiv). The resulting mixture at 100° C. under nitrogen atmosphere was stirred overnight. The resulting mixture was diluted with water (50 mL). The resulting mixture was extracted with EtOAc (3×50 mL), dried over anhydrous Na₂SO₄. After filtration, the filtrate was concentrated under reduced pressure. The residue was purified by silica gel column chromatography, eluted with PE:EA (2:1) to afford the title compound as brown oil (400 mg, 78.1%). LCMS: m/z=292 [M+H]⁺

### Preparation 130: 4-(1-cyclopropyl-2-oxo-5-phenyl-1,2-dihydropyridin-4-yl)-2-(2,6-dimethylpyridin-4-yl)-6-methyl-1-tosyl-1,6-dihydro-7H-pyrrolo[2,3-c]pyridin-7-one

To a stirred mixture of (2-(2,6-dimethylpyridin-4-yl)-6-methyl-7-oxo-1-tosyl-6,7-dihydro-1H-pyrrolo[2,3-c]pyridin-4-yl)boronic acid (250 mg, 0.553 mmol, 1.00 equiv) and 4-bromo-1-cyclopropyl-5-phenylpyridin-2 (1H)-one (242 mg, 0.829 mmol, 1.50 equiv) in DME (5.0 mL) and H₂O (1.0 mL) at room temperature was added Pd(PPh₃)₄ (63.9 mg, 0.055 mmol, 0.100 equiv) and Na₂CO₃ (117 mg, 1.11 mmol, 2.00 equiv). The resulting mixture was stirred at 60° C. for 3 h. The resulting mixture was quenched with water. The resulting mixture was extracted with EtOAc (3×10 mL), dried over anhydrous Na₂SO₄. After filtration, the filtrate was concentrated under reduced pressure. This resulted in the title compound as yellow solid (450 mg, crude). LCMS: m/z=617 [M+H]⁺

### Preparation 131: 4-(1-cyclopropyl-2-oxo-5-phenyl-1,2-dihydropyridin-4-yl)-2-(2,6-dimethylpyridin-4-yl)-6-methyl-1,6-dihydro-7H-pyrrolo[2,3-c]pyridin-7-one hydrogen chloride

To a stirred mixture of 4-(1-cyclopropyl-2-oxo-5-phenyl-1,2-dihydropyridin-4-yl)-2-(2,6-dimethylpyridin-4-yl)-6-methyl-1-tosyl-1,6-dihydro-7H-pyrrolo[2,3-c]pyridin-7-one (450 mg, 0.730 mmol, 1.00 equiv.) in MeOH (5.0 mL) and H₂O (1.0 mL) at room temperature was added NaOH (292 mg, 7.30 mmol, 10.0 equiv). The resulting mixture at 60° C. was stirred for 2 h. The mixture was allowed to cool down to room temperature. The reaction was quenched by water. The resulting mixture was extracted with EtOAc (3×10 mL). The combined organic layers were washed with brine, dried over anhydrous Na₂SO₄. After filtration, the filtrate was concentrated under reduced pressure. The residue was purified by RP-HPLC^{AV} to afford the product. The product was further treated with HCl in MeOH (2.0 mL, 4.0 M/L) and lyophilized to afford the title compound as yellow solid (73.4 mg, 21.8%). ¹H NMR (400 MHz, Methanol-d₄) δ 7.96 (s, 2H), 7.79 (s, 1H), 7.30-7.24 (m, 2H), 7.23-7.12 (m, 4H), 6.96 (s, 1H), 6.81 (s, 1H), 3.56 (s, 3H), 3.55-3.49 (m, 1H), 2.75 (s, 6H), 1.30-1.20 (m, 2H), 1.14-1.02 (m, 2H). LCMS^{V}: m/z=463 [M+H]⁺

### Example 29: 4-(4-(2-(2,6-dimethylpyridin-4-yl)-6-methyl-7-oxo-6,7-dihydro-1H-pyrrolo[2,3-c]pyridin-4-yl)-1-methyl-6-oxo-1,6-dihydropyridin-3-yl)benzonitrile hydrogen chloride

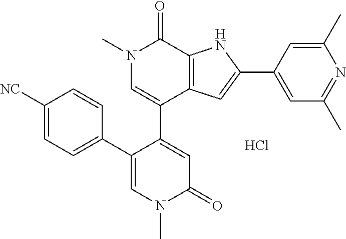 [SMILES: Cc1cc(-c2cc3c(-c4cc(=O)n(C)cc4-c4ccc(C#N)cc4)cn(C)c(=O)c3[nH]2)cc(C)n1 ; Cl]

### Preparation 132: 4-(4-bromo-1-methyl-6-oxo-1,6-dihydropyridin-3-yl)benzonitrile

To a mixture of 4-bromo-5-iodo-1-methylpyridin-2 (1H)-one (5.00 g, 15.9 mmol, 1.00 equiv) and (4-cyanophenyl)boronic acid (4.68 g, 31.9 mmol, 2.00 equiv) in DMF (50.0 mL) and H₂O (5.0 mL) at room temperature under nitrogen atmosphere was added Na₂CO₃ (3.38 g, 31.9 mmol, 2.00 equiv) and Pd(PPh₃)₄ (1.84 g, 1.59 mmol, 0.100 equiv). The resulting mixture at 100° C. was stirred for 4 h. The resulting mixture was extracted with EtOAc (3×50 mL), dried over anhydrous Na₂SO₄. After filtration, the filtrate was concentrated under reduced pressure. The residue was purified by silica gel column chromatography, eluted with PE/EA (1:1) to afford the title compound as a yellow solid (2.40 g, 52.1%). LCMS: m/z=291 [M+H]⁺

### Preparation 133: 4-(4-(2-(2,6-dimethylpyridin-4-yl)-6-methyl-7-oxo-1-tosyl-6,7-dihydro-1H-pyrrolo[2, 3-c]pyridin-4-yl)-1-methyl-6-oxo-1,6-dihydropyridin-3-yl)benzonitrile

To a mixture of (2-(2,6-dimethylpyridin-4-yl)-6-methyl-7-oxo-1-tosyl-6,7-dihydro-1H-pyrrolo[2,3-c]pyridin-4-yl)boronic acid (200 mg, 0.443 mmol, 1.00 equiv) and 4-(4-bromo-1-methyl-6-oxo-1,6-dihydropyridin-3-yl)benzonitrile (259 mg, 0.898 mmol, 2.03 equiv) in 1,4-dioxane (10.0 mL) and H₂O (2.0 mL) at room temperature was added Na₂CO₃ (93.9 mg, 0.886 mmol, 2.00 equiv) and XPhos Pd G3 (50.0 mg, 0.059 mmol, 0.130 equiv). The resulting mixture at 60° C. under nitrogen atmosphere was stirred overnight. The resulting mixture was extracted with EtOAc (3×15 mL). The combined organic layers were washed with brine, dried over anhydrous Na₂SO₄. After filtration, the filtrate was concentrated under reduced pressure. The residue was purified by Prep-TLC (EA/MeOH=10:1) to afford the title compound as a yellow solid (160 mg, 80.0%). LCMS: m/z=616 [M+H]⁺.

### Preparation 134: 4-(4-(2-(2,6-dimethylpyridin-4-yl)-6-methyl-7-oxo-6,7-dihydro-1H-pyrrolo[2,3-c]pyridin-4-yl)-1-methyl-6-oxo-1,6-dihydropyridin-3-yl)benzonitrile hydrochloride

To a mixture of 4-(4-(2-(2,6-dimethylpyridin-4-yl)-6-methyl-7-oxo-1-tosyl-6,7-dihydro-1H-pyrrolo[2,3-c]pyridin-4-yl)-1-methyl-6-oxo-1,6-dihydropyridin-3-yl)benzonitrile (200 mg, 0.325 mmol, 1.00 equiv) in MeOH (2.0 mL) and H₂O (0.5 mL) at room temperature was added NaOH (129 mg, 3.25 mmol, 10.0 equiv). The resulting mixture at 40° C. was stirred for 2 h. The resulting mixture was extracted with EtOAc (3×5.0 mL). The combined organic layers were washed with brine, dried over anhydrous Na₂SO₄. After filtration, the filtrate was concentrated under reduced pressure.

The residue was purified by silica gel column chromatography, eluted with PE/EA (1:1) to afford the product. The product was further treated with HCl in MeOH (2.0 mL, 4.0 M) and lyophilized to afford the title compound as a yellow solid (13.0 mg, 8.7%). ¹H NMR (400 MHz, Methanol-d⁴) δ 7.95 (d, J=2.7 Hz, 3H), 7.53 (d, J=8.3 Hz, 2H), 7.44 (d, J=8.3 Hz, 2H), 7.25 (s, 1H), 6.86 (s, 1H), 6.73 (s, 1H), 3.71 (s, 3H), 3.61 (s, 3H), 2.73 (s, 6H).

LCMS^{W}: m/z=462 [M+H]⁺.

### Example 30: 2-(2,6-dimethylpyridin-4-yl)-4-(5-(4-fluorophenyl)-1-methyl-2-oxo-1,2-dihydropyridin-4-yl)-6-methyl-1,6-dihydro-7H-pyrrolo[2,3-c]pyridin-7-one hydrochloride (BA62)

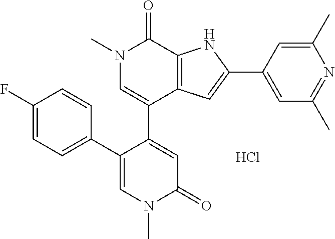 [SMILES: Cc1cc(-c2cc3c(-c4cc(=O)n(C)cc4-c4ccc(F)cc4)cn(C)c(=O)c3[nH]2)cc(C)n1 ; Cl]

### Preparation 135: 4-bromo-5-(4-fluorophenyl)-1-methylpyridin-2 (1H)-one

To a stirred mixture of 4-bromo-5-iodo-1-methylpyridin-2 (1H)-one (5.00 g, 15.9 mmol, 1.00 equiv) and 4-fluorophenylboronic acid (3.12 g, 22.3 mmol, 1.40 equiv) in DMF (30.0 mL) and H₂O (3.0 mL) at room temperature was added Na₂CO₃ (3.38 g, 31.9 mmol, 2.00 equiv) and Pd(PPh₃)₄ (920 mg, 0.796 mmol, 0.05 equiv). The resulting mixture at 120° C. under nitrogen atmosphere was stirred for 6 h. The resulting mixture was extracted with EtOAc (3×50 mL). The combined organic layers were washed with brine, dried over anhydrous Na₂SO₄. After filtration, the filtrate was concentrated under reduced pressure. The residue was purified by RP-HPLC^{AX} to afford the title compound (3.00 g, 60.8%) as a brown solid. LCMS: m/z=284 [M+H]⁺

### Preparation 136: 2-(2,6-dimethylpyridin-4-yl)-4-(5-(4-fluorophenyl)-1-methyl-2-oxo-1,2-dihydropyridin-4-yl)-6-methyl-1-tosyl-1,6-dihydro-7H-pyrrolo[2,3-c]pyridin-7-one

To a mixture of (2-(2,6-dimethylpyridin-4-yl)-6-methyl-7-oxo-1-tosyl-6,7-dihydro-1H-pyrrolo[2,3-c]pyridin-4-yl)boronic acid (179 mg, 0.397 mmol, 1.00 equiv) and 4-bromo-5-(4-fluorophenyl)-1-methylpyridin-2 (1H)-one (223.8 mg, 0.794 mmol, 2.00 equiv) in dioxane (10.0 mL) and H₂O (2.0 mL) at room temperature was added Na₂CO₃ (84.1 mg, 0.794 mmol, 2.00 equiv) and XPhos Pd G3 (33.6 mg, 0.040 mmol, 0.10 equiv). The resulting mixture at 60° C. under nitrogen atmosphere was stirred for 5 h. The reaction was quenched by water at room temperature. The resulting mixture was extracted with EtOAc (3×10 mL). The combined organic layers were washed with brine, dried over anhydrous Na₂SO₄. After filtration, the filtrate was concentrated under reduced pressure. The residue was purified by Prep-TLC (EA I MeOH 15:1) to afford the title compound as a yellow solid (97.3 mg, 40.3%). LCMS: m/z=609 [M+H]⁺.

### Preparation 137: 2-(2,6-dimethylpyridin-4-yl)-4-(5-(4-fluorophenyl)-1-methyl-2-oxo-1,2-di hydropyridin-4-yl)-6-methyl-1,6-dihydro-7H-pyrrolo[2,3-c]pyridin-7-one hydrochloride

To a mixture of 2-(2,6-dimethylpyridin-4-yl)-4-(5-(4-fluorophenyl)-1-methyl-2-oxo-1,2-dihydropyridin-4-yl)-6-methyl-1-tosyl-1,6-dihydro-7H-pyrrolo[2,3-c]pyridin-7-one (90.0 mg, 0.148 mmol, 1.00 equiv) in MeOH (4.0 mL) and H₂O (1.0 mL) at room temperature was added NaOH (59.1 mg, 1.48 mmol, 10.0 equiv). The resulting mixture at 40° C. was stirred for 1 h. The reaction was quenched by water. The resulting mixture was extracted with EtOAc (5×10 mL). The combined organic layers were washed with brine, dried over anhydrous Na₂SO₄. After filtration, the filtrate was concentrated under reduced pressure. The crude product was purified by Prep-HPLC^{AY} to afford the product. The product was further treated with HCl in MeOH (2.0 mL, 4.0 M) and lyophilized to afford the title compound as a yellow solid (34.0 mg, 50.2%). ¹H NMR (300 MHz, DMSO-d⁶) δ 8.00 (d, J=3.5 Hz, 2H), 7.86 (s, 1H), 7.31-7.18 (m, 3H), 6.95 (t, J=8.3 Hz, 2H), 6.79 (s, 1H), 6.55 (s, 1H), 3.55 (s, 3H), 3.49 (s, 3H), 2.60 (s, 6H). LCMS^{X}: m/z=455 [M+H]⁺.

### Example 31: 2-(2,6-dimethylpyridin-4-yl)-4-(5-(4-methoxyphenyl)-1-methyl-2-oxo-1,2-dihydropyridin-4-yl)-6-methyl-1,6-dihydro-7H-pyrrolo[2,3-c]pyridin-7-one hydrogen chloride

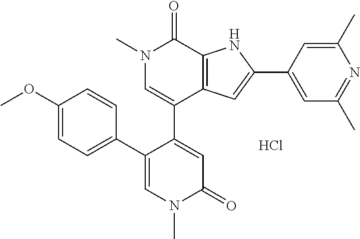

### Preparation 138: 4-bromo-5-(4-methoxyphenyl)-1-methylpyridin-2 (1H)-one

To a stirred mixture of 4-bromo-5-iodo-1-methylpyridin-2 (1H)-one (5.00 g, 15.9 mmol, 1.00 equiv) and (4-methoxyphenyl)boronic acid (3.63 g, 23.9 mmol, 1.50 equiv) at room temperature in DMF (30.0 mL) and H₂O (3.0 mL) was added Na₂CO₃ (3.37 g, 31.8 mmol, 2.00 equiv) and Pd(PPh₃)₄ (920 mg, 0.796 mmol, 0.05 equiv). The resulting mixture at 100° C. under nitrogen atmosphere was stirred for 6 h. The resulting mixture was extracted with EtOAc (3×100 mL). The combined organic layers were washed with brine, dried over anhydrous Na₂SO₄. After filtration, the filtrate was concentrated under reduced pressure. The residue was purified by RP-HPLC^{AR} to afford the title compound as a brown solid (2.50 g, 53.4%). LCMS: m/z=296 [M+H]⁺.

### Preparation 139: 2-(2,6-dimethylpyridin-4-yl)-4-(5-(4-methoxyphenyl)-1-methyl-2-oxo-1,2-dihydropyridin-4-yl)-6-methyl-1-tosyl-1,6-dihydro-7H-pyrrolo[2,3-c]pyridin-7-one

To a mixture of (2-(2,6-dimethylpyridin-4-yl)-6-methyl-7-oxo-1-tosyl-6,7-dihydro-1H-pyrrolo[2,3-c]pyridin-4-yl)boronic acid (200 mg, 0.443 mmol, 1.00 equiv) and 4-bromo-5-(4-methoxyphenyl)-1-methylpyridin-2 (1H)-one (261 mg, 0.886 mmol, 2.00 equiv) in 1,4-dioxane (10.0 mL) and H₂O (2.00 mL) at room temperature was added Na₂CO₃ (93.9 mg, 0.886 mmol, 2.00 equiv) and XPhos Pd G3 (37.5 mg, 0.0440 mmol, 0.100 equiv). The resulting mixture at 60° C. under nitrogen atmosphere was stirred overnight. The reaction at room temperature was quenched by the addition of water. The resulting mixture was extracted with EtOAc (6×30 mL). The combined organic layers were washed with brine, dried over anhydrous Na₂SO₄. After filtration, the filtrate was concentrated under reduced pressure. The residue was purified by Prep-TLC (EA/MeOH 10:1) to afford the title compound as a yellow solid (121 mg, 44.0%). LCMS: m/z=621 [M+H]⁺.

### Preparation 140: 2-(2,6-dimethylpyridin-4-yl)-4-(5-(4-methoxyphenyl)-1-methyl-2-oxo-1,2-dihydropyridin-4-yl)-6-methyl-1,6-dihydro-7H-pyrrolo[2, 3-c]pyridin-7-one

To a mixture of 2-chloro-4-(5-(4-methoxyphenyl)-1-methyl-2-oxo-1,2-dihydropyridin-4-yl)-6-methyl-1-tosyl-1,6-dihydro-7H-pyrrolo[2,3-c]pyridin-7-one (110 mg, 0.177 mmol, 1 equiv) in MeOH (4.0 mL) and H₂O (1.0 mL) at room temperature was added NaOH (70.9 mg, 1.77 mmol, 10.0 equiv). The resulting mixture was stirred at 40° C. for 1 h. The reaction was quenched by the addition of water at room temperature. The resulting mixture was extracted with EtOAc (3×10 mL). The combined organic layers were washed with brine, dried over anhydrous Na₂SO₄. After filtration, the filtrate was concentrated under reduced pressure. The crude product was purified by Prep-HPLC^{BL} to afford the product. The product was further treated with HCl in MeOH (2.0 mL, 4.0 M) and lyophilized to afford the title compound as a yellow solid (56.7 mg, 67.8%). ¹H NMR (400 MHz, DMSO-d₆) δ 15.36 (s, 1H), 12.93 (d, J=2.3 Hz, 1H), 8.18 (s, 2H), 7.86 (s, 1H), 7.21 (s, 1H), 7.12 (d, J=8.5 Hz, 2H), 6.96 (d, J=2.3 Hz, 1H), 6.74 (d, J=8.5 Hz, 2H), 6.49 (s, 1H), 3.61 (s, 3H), 3.55 (s, 3H), 3.48 (s, 3H), 2.66 (s, 6H). LCMS^{Y}: m/z=467 [M+H]⁺.

### Example 32: 4-(5-(2-chlorophenyl)-1-methyl-2-oxo-1,2-dihydropyridin-4-yl)-2-(2,6-dimethylpyridin-4-yl)-6-methyl-1,6-dihydro-7H-pyrrolo[2,3-c]pyridin-7-one hydrogen chloride

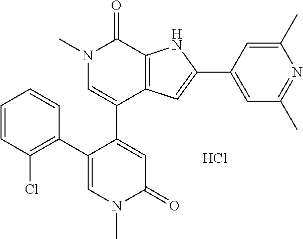 [SMILES: Cc1cc(-c2cc3c(-c4cc(=O)n(C)cc4-c4ccccc4Cl)cn(C)c(=O)c3[nH]2)cc(C)n1 ; Cl]

### Preparation 141: 4-bromo-5-(2-chlorophenyl)-1-methylpyridin-2 (1H)-one

To a stirred mixture of 4-bromo-5-iodo-1-methylpyridin-2 (1H)-one (3.00 g, 9.56 mmol, 1.00 equiv) and 2-chlorophenylboronic acid (2.99 g, 19.1 mmol, 2.00 equiv) in DMF (30.0 mL) and H₂O (6.0 mL) at room temperature under nitrogen atmosphere was added Na₂CO₃ (2.03 g, 19.1 mmol, 2.00 equiv) and Pd(PPh₃)₄ (1.10 g, 0.956 mmol, 0.100 equiv). The resulting mixture was stirred at 100° C. under nitrogen atmosphere for 4 h. The reaction was quenched with water at room temperature. The resulting mixture was extracted with EtOAc (3×60 mL). The combined organic layers were washed with brine, dried over anhydrous Na₂SO₄. After filtration, the filtrate was concentrated under reduced pressure. The residue was purified by silica gel column chromatography, eluted with PE/EA (1:1) to afford the title compound as a yellow solid (1.54 g, 54.0%). LCMS: m/z=300 [M+H]⁺

### Preparation 142: 4-(5-(2-chlorophenyl)-1-methyl-2-oxo-1,2-dihydropyridin-4-yl)-2-(2,6-dimethylpyridin-4-yl)-6-methyl-1-tosyl-1,6-dihydro-7H-pyrrolo[2,3-c]pyridin-7-one

To a mixture of 4-bromo-5-(2-chlorophenyl)-1-methylpyridin-2 (1H)-one (329 mg, 1.10 mmol, 1.00 equiv) and (2-(2,6-dimethylpyridin-4-yl)-6-methyl-7-oxo-1-tosyl-6,7-dihydro-1H-pyrrolo[2,3-c]pyridin-4-yl)boronic acid (828 mg, 1.67 mmol, 1.50 equiv) in DME (12.5 mL) and H₂O (2.5 mL) at room temperature was added Pd(dppf)Cl₂·CH₂Cl₂ (89.8 mg, 0.080 mmol, 0.100 equiv) and Na₂CO₃ (233 mg, 2.20 mmol, 2.00 equiv). The resulting mixture at 60° C. under nitrogen atmosphere was stirred overnight. The resulting mixture was extracted with EtOAc (3×20 mL). The combined organic layers were washed with brine, dried over anhydrous Na₂SO₄. After filtration, the filtrate was concentrated under reduced pressure. This resulted in the title compound as a yellow oil (380 mg, crude). The crude product was used in the next step directly without further purification. LCMS: m/z=625 [M+H]⁺.

### Preparation 143: 4-(5-(2-chlorophenyl)-1-methyl-2-oxo-1,2-dihydropyridin-4-yl)-2-(2,6-dimethylpyridin-4-yl)-6-methyl-1,6-dihydro-7H-pyrrolo[2, 3-c]pyridin-7-one hydrochloride

To a mixture of 4-(5-(2-chlorophenyl)-1-methyl-2-oxo-1,2-dihydropyridin-4-yl)-2-(2,6-dimethylpyridin-4-yl)-6-methyl-1-tosyl-1,6-dihydro-7H-pyrrolo[2,3-c]pyridin-7-one (360 mg, 0.576 mmol, 1.00 equiv) in MeOH (4.0 mL) and H₂O (1.0 mL) at room temperature was added NaOH (230 mg, 5.77 mmol, 10.0 equiv). The resulting mixture at 60° C. was stirred for 3 h. The resulting mixture was extracted with EtOAc (3×10 mL). The combined organic layers were washed with brine, dried over anhydrous Na₂SO₄. After filtration, the filtrate was concentrated under reduced pressure. The residue was purified by silica gel column chromatography, eluted with PE/EA (1:1) and further purified by Prep-HPLC^{AZ} to afford the product. The product was further treated with HCl in MeOH (2.0 mL, 4.0 M) and lyophilized to afford the tittle compound as a light-yellow solid (28.0 mg, 9.7%). ¹H NMR (400 MHz, Methanol-d⁴) δ 8.05 (s, 2H), 7.86 (s, 1H), 7.48-7.38 (m, 1H), 7.37-7.17 (m, 4H), 6.88 (s, 1H), 6.83 (s, 1H), 3.71 (d, J=10.5 Hz, 3H), 3.43 (s, 3H), 2.76 (s, 6H). LCMS^{Z}: m/z=471 [M+H]⁺.

### Example 33: 4-(5-(1,3-dihydroisobenzofuran-5-yl)-1-methyl-2-oxo-1,2-dihydropyridin-4-yl)-2-(2,6-dimethylpyridin-4-yl)-6-methyl-1,6-dihydro-7H-pyrrolo[2,3-c]pyridin-7-one hydrochloride

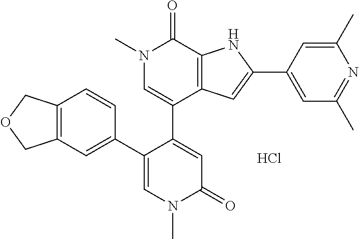 [SMILES: Cc1cc(-c2cc3c(-c4cc(=O)n(C)cc4-c4ccc5c(c4)COC5)cn(C)c(=O)c3[nH]2)cc(C)n1 ; Cl]

### Preparation 144: (5-bromo-2-(chloromethyl)phenyl)methanol

To a solution of (4-bromo-1,2-phenylene)dimethanol (5.0 g, 23.0 mmol, 1.00 equiv) at room temperature was added HCl (12 M, 25 mL). The resulting mixture at 70° C. was stirred for 1 h. The reaction was quenched by the addition of water/ice (10 mL) at room temperature. The resulting mixture was extracted with EtOAc (2×20 mL). The combined organic layers were washed with brine, dried over anhydrous Na₂SO₄. After filtration, the filtrate was concentrated under reduced pressure. The residue was purified by silica gel column chromatography, eluted with PE/EA (8:1) to afford the title compound as a white solid (3.20 g, 59.0%). ¹H NMR (300 MHz, DMSO-d₆) δ 7.64 (d, J=2.2 Hz, 1H), 7.52-7.43 (m, 1H), 7.36 (d, J=8.1 Hz, 1H), 5.40-5.45 (m, 1H), 4.78 (s, 2H), 4.66 (m, 2H).

### Preparation 145: 5-bromo-1,3-dihydroisobenzofuran

To a solution of (5-bromo-2-(chloromethyl)phenyl)methanol (3.21 g, 13.6 mmol, 1.00 equiv) in THF (50 mL) at 0° C. was added with NaH (1.08 g, 27.2 mmol, 2.00 equiv, 60% in mineral oil) for 5 min. The resulting mixture at 45° C. was stirred for additional 2 h. The reaction was quenched by the addition of ice/water (150 mL) at room temperature. The resulting mixture was extracted with EtOAc (5×100 mL). The combined organic layers were washed with brine, dried over anhydrous Na₂SO₄. After filtration, the filtrate was concentrated under reduced pressure. The residue was purified by silica gel column chromatography, eluted with PE to afford the title compound as a white solid (1.76 g, 65.1%). ¹H NMR (400 MHz, DMSO-d⁶) δ 7.54 (s, 1H), 7.48-7.43 (m, 1H), 7.28 (d, J=8.0 Hz, 1H), 5.04-4.91 (m, 4H).

### Preparation 146: 2-(1,3-dihydroisobenzofuran-5-yl)-4,4,5,5-tetramethyl-1,3,2-dioxaborolane

To a stirred mixture of 5-bromo-1,3-dihydro-2-benzofuran (2.00 g, 10.0 mmol, 1.00 equiv) and 4,4,4′,4′,5,5,5′,5′-octamethyl-2,2′-bi(1,3,2-dioxaborolane) (10.2 g, 40.2 mmol, 4.00 equiv) in dioxane (20.0 mL) at room temperature under nitrogen atmosphere was added KOAc (1.97 g, 20.1 mmol, 2.00 equiv) and Pd(dppf)Cl₂·CH₂Cl₂ (0.820 g, 1.01 mmol, 0.100 equiv). The resulting mixture was stirred at 80° C. under nitrogen atmosphere for 4 h. The reaction was quenched with water at room temperature. The resulting mixture was extracted with EtOAc (3×15 mL). The combined organic layers were washed with brine, dried over anhydrous Na₂SO₄. After filtration, the filtrate was concentrated under reduced pressure. The residue was purified by silica gel column chromatography, eluted with PE/EA (5:1) to afford the crude product as a white crude solid (8.90 g), and then the crude was further purified by RP-HPLCB^{A} to afford the title compound as a white solid (1.20 g, 48.5%). 1H NMR (400 MHz, Chloroform-d) δ 7.84-7.63 (m, 2H), 7.28 (s, 1H), 5.21-5.06 (m, 4H), 1.37 (s, 12H).

### Preparation 147: 4-bromo-5-(1,3-dihydroisobenzofuran-5-yl)-1-methylpyridin-2 (1H)-one

To a stirred mixture of 2-(1,3-dihydroisobenzofuran-5-yl)-4,4,5,5-tetramethyl-1,3,2-dioxaborolane (635 mg, 2.58 mmol, 1.50 equiv) and 4-bromo-5-iodo-1-methylpyridin-2 (1H)-one (540 mg, 1.72 mmol, 1.00 equiv) in DMF (10.0 mL) and H₂O (1.00 mL) at room temperature was added Pd(PPh₃)₄ (198 mg, 0.172 mmol, 0.100 equiv) and Na₂CO₃ (365 mg, 3.44 mmol, 2.00 equiv). The resulting mixture was stirred at 80° C. under nitrogen atmosphere for 6 h. The reaction was quenched with water at room temperature. The resulting mixture was extracted with EtOAc (3×50 mL). The combined organic layers were washed with brine, dried over anhydrous Na₂SO₄. After filtration, the filtrate was concentrated under reduced pressure. The residue was purified by silica gel column chromatography, eluted with PE/EA (8:1) to afford the title compound as a yellow solid (330 mg, 62.7%). LCMS: m/z=308 [M+H]⁺

### Preparation 148: 4-(5-(1,3-dihydroisobenzofuran-5-yl)-1-methyl-2-oxo-1,2-dihydropyridin-4-yl)-2-(2,6-dimethylpyridin-4-yl)-6-methyl-1-tosyl-1,6-dihydro-7H-pyrrolo[2,3-c]pyridin-7-one

To a mixture of (2-(2,6-dimethylpyridin-4-yl)-6-methyl-7-oxo-1-tosyl-6,7-dihydro-1H-pyrrolo[2,3-c]pyridin-4-yl)boronic acid (184 mg, 0.408 mmol, 1.00 equiv) and 4-bromo-5-(1,3-dihydroisobenzofuran-5-yl)-1-methylpyridin-2 (1H)-one (250 mg, 0.817 mmol, 2.00 equiv) in DME (5.0 mL) and H₂O (1.0 mL) at room temperature was added Pd(dppf)Cl₂·CH₂Cl₂ (33.3 mg, 0.041 mmol, 0.100 equiv) and Na₂CO₃ (86.5 mg, 0.817 mmol, 2.00 equiv). The resulting mixture at 60° C. under nitrogen atmosphere was stirred for 6 h. The reaction was quenched with water at room temperature. The resulting mixture was extracted with EtOAc (3×10 mL). The combined organic layers were washed with brine, dried over anhydrous Na₂SO₄. After filtration, the filtrate was concentrated under reduced pressure. The resulted in the title compound (376 mg) as a yellow crude solid used in the next step directly without further purification. LCMS: m/z=633 [M+H]⁺

### Preparation 149: 4-(5-(1,3-dihydroisobenzofuran-5-yl)-1-methyl-2-oxo-1,2-dihydropyridin-4-yl)-2-(2,6-dimethylpyridin-4-yl)-6-methyl-1,6-dihydro-7H-pyrrolo[2,3-c]pyridin-7-one hydrochloride

To a stirred mixture of 4-(5-(1,3-dihydroisobenzofuran-5-yl)-1-methyl-2-oxo-1,2-dihydropyridin-4-yl)-2-(2,6-dimethylpyridin-4-yl)-6-methyl-1-tosyl-1,6-dihydro-7H-pyrrolo[2,3-c]pyridin-7-one (400 mg, 0.632 mmol, 1.00 equiv) in MeOH (4.0 mL) and H₂O (1.0 mL) at room temperature was added NaOH (253 mg, 6.32 mmol, 10.0 equiv). The resulting mixture at 60° C. was stirred for 1 h. The reaction was quenched with water at room temperature. The resulting mixture was extracted with EtOAc (3×10 mL). The combined organic layers were washed with brine, dried over anhydrous Na₂SO₄. After filtration, the filtrate was concentrated under reduced pressure. The crude product was purified by Prep-HPLCB^{B} to afford the product. The product was further treated with HCl in MeOH (2.0 mL, 4.0 M) and lyophilized to afford the title compound as a yellow solid (41.1 mg, 12.9%). H NMR (400 MHz, DMSO-d⁶) δ 15.57 (s, 1H), 12.98 (d, J=2.4 Hz, 1H), 8.18 (s, 2H), 7.90 (s, 1H), 7.17-6.98 (m, 5H), 6.56 (s, 1H), 4.91 (s, 4H), 3.54 (s, 3H), 3.41 (s, 3H), 2.68 (s, 6H). LCMS^{AA}: m/z=479 [M+H]⁺.

### Example 34 (Compound A):6-methyl-4-(1-methyl-2-oxo-5-phenyl-1,2-dihydropyridin-4-yl)-2-(1-(trifluoromethyl)-1H-pyrazol-4-yl)-1,6-dihydro-7H-pyrrolo[2,3-c]pyridin-7-one

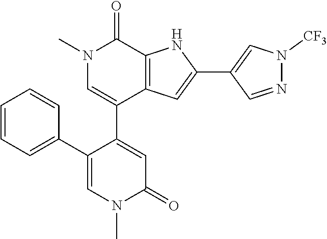 [SMILES: Cn1cc(-c2ccccc2)c(-c2cn(C)c(=O)c3[nH]c(-c4cnn(C(F)(F)F)c4)cc23)cc1=O]

### Preparation 150: 6-methyl-4-(1-methyl-2-oxo-5-phenyl-1,2-dihydropyridin-4-yl)-2-(1-(trifluoromethyl)-1H-pyrazol-4-yl)-1,6-dihydro-7H-pyrrolo[2,3-c]pyridin-7-one

To a stirred mixture of 2-chloro-6-methyl-4-(1-methyl-2-oxo-5-phenyl-1,2-dihydropyridin-4-yl)-1,6-dihydro-7H-pyrrolo[2,3-c]pyridin-7-one (150 mg, 0.410 mmol, 1.00 equiv) and 4-(4,4,5,5-tetramethyl-1,3,2-dioxaborolan-2-yl)-1-(trifluoromethyl)-1H-pyrazole (215 mg, 0.820 mmol, 2.00 equiv) in DME (7.5 mL) and H₂O (1.5 mL) at room temperature were added XPhos Pd G3 (34.7 mg, 0.0410 mmol, 0.100 equiv) and K₂CO₃ (113 mg, 0.820 mmol, 2.00 equiv). The resulting mixture was stirred at 80° C. under a nitrogen atmosphere for 4 h. The reaction was quenched with water. The resulting mixture was extracted with EtOAc (4×15 mL). The combined organic layers were washed with brine (2×50 mL), dried over anhydrous Na₂SO₄. After filtration, the filtrate was concentrated under reduced pressure. The crude product was purified by Prep-HPLC^{B} to afford the title compound as a white solid (124 mg, 65%). LCMSA: m/z=466 [M+H]⁺. ¹H NMR (400 MHz, CDCl₃) δ 12.58 (s, 1H), 8.56 (s, 1H), 8.21 (s, 1H), 7.42 (s, 1H), 7.29-7.18 (m, 3H), 7.17-7.10 (m, 2H), 6.85 (s, 1H), 6.59 (s, 1H), 6.39 (s, 1H), 3.68 (s, 3H), 3.50 (s, 3H).

### Example 35: 2-(2,4-difluorophenyl)-6-methyl-4-(1-methyl-5-(3-(oxetan-3-yl)phenyl)-2-oxo-1,2-dihydropyridin-4-yl)-1,6-dihydro-7H-pyrrolo[2,3-c]pyridin-7-one

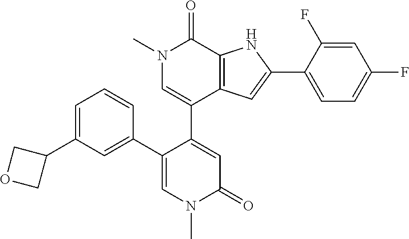 [SMILES: Cn1cc(-c2cccc(C3COC3)c2)c(-c2cn(C)c(=O)c3[nH]c(-c4ccc(F)cc4F)cc23)cc1=O]

### Preparation 151: 3-(3-bromophenyl)oxetane

To a stirred mixture of 3-bromophenylboric acid (5.00 g, 25.0 mmol, 1.25 equiv), trans-2-aminocyclohexanol hydrochloride (182 mg, 1.20 mmol, 0.060 equiv) and NiI₂ (396 mg, 1.20 mmol, 0.060 equiv) in i-PrOH (100 mL) at room temperature was added sodium hexamethyldisilazane (37.4 mL, 37.4 mmol, 1.87 equiv) under nitrogen atmosphere. The resulting mixture was stirred for 10 min at room temperature under nitrogen atmosphere. To the above mixture was added 3-iodooxetane (3.68 g, 20.0 mmol, 1.00 equiv) at room temperature. The resulting mixture was stirred for additional overnight at 90° C. The resulting mixture was diluted with EtOH (100 mL). The resulting mixture was filtered, the filter cake was washed with EtOH. The filtrate was concentrated under reduced pressure. The residue was purified by silica gel column chromatography, eluted with PE to afford the title compound as a light-yellow oil (797 mg, 18.7%). ¹H NMR (300 MHz, Chloroform-d) δ 7.56 (t, J=1.9 Hz, 1H), 7.47-7.37 (m, 1H), 7.37-7.30 (m, 1H), 7.26-7.17 (m, 1H), 5.08 (dd, J=8.4, 6.1 Hz, 2H), 4.74 (t, J=6.3 Hz, 2H), 4.27-4.10 (m, 1H).

### Preparation 152: 4,4,5,5-tetramethyl-2-(3-(oxetan-3-yl)phenyl)-1,3,2-dioxaborolane

To a stirred mixture of 3-(3-bromophenyl)oxetane (660 mg, 3.11 mmol, 1.00 equiv) and bis(pinacolato)diboron (3.16 g, 12.5 mmol, 4.00 equiv) in dioxane (35.0 mL) at room temperature was added KOAc (610 mg, 6.22 mmol, 2.00 eq) and Pd(dppf)Cl₂·CH₂Cl₂ (254 mg, 0.311 mmol, 0.100 equiv). The resulting mixture was stirred at 80° C. under nitrogen atmosphere overnight. The resulting mixture was extracted with EtOAc (3×5 mL). The combined organic layers were washed with brine, dried over anhydrous Na₂SO₄. After filtration, the filtrate was concentrated under reduced pressure. The residue was purified by silica gel column chromatography, eluted with PE/EA (1:10) to afford the tittle compound as a white oil (210 mg, crude).

### Preparation 153: 4-bromo-1-methyl-5-(3-(oxetan-3-yl)phenyl)pyridin-2 (1H)-one

To a stirred solution of 4-bromo-5-iodo-1-methylpyridin-2 (1H)-one (732 mg, 2.33 mmol, 1.00 equiv) and 4,4,5,5-tetramethyl-2-(3-(oxetan-3-yl)phenyl)-1,3,2-dioxaborolane (727 mg, 2.79 mmol, 1.20 equiv) in DMF (5.0 mL) and H₂O (0.5 mL) at room temperature was added Pd(PPh₃)₄ (269 mg, 0.233 mmol, 0.10 equiv) and Na₂CO₃ (494 mg, 4.66 mmol, 2.00 equiv). The resulting mixture was stirred at 80° C. under a nitrogen atmosphere for 1 h. The resulting mixture was extracted with EtOAc (3×30 mL). The combined organic layers were washed with brine, dried over anhydrous Na₂SO₄. After filtration, the filtrate was concentrated under reduced pressure. The residue was purified by silica gel column chromatography, eluted with PE/EA (1:1) to afford the title compound as a brown solid (361 mg, 48.4%). LCMS: m/z=322 [M+H]⁺

### Preparation 154: 2-chloro-6-methyl-4-(1-methyl-5-(3-(oxetan-3-yl)phenyl)-2-oxo-1,2-dihydropyridin-4-yl)-1-tosyl-1,6-dihydro-7H-pyrrolo[2,3-c]pyridin-7-one

To a mixture of 4-bromo-1-methyl-5-(3-(oxetan-3-yl)phenyl)pyridin-2 (1H)-one (350 mg, 1.10 mmol, 1.00 equiv) and 2-chloro-4-(5,5-dimethyl-1,3,2-dioxaborinan-2-yl)-6-methyl-1-tosyl-1,6-dihydro-7H-pyrrolo[2,3-c]pyridin-7-one (736 mg, 1.64 mmol, 1.50 equiv) in DME (10 mL) and H₂O (2 mL) at room temperature was added Pd(dppf)Cl₂·CH₂Cl₂ (80.0 mg, 0.109 mmol, 0.100 equiv) and Na₂CO₃ (232 mg, 2.19 mmol, 2.00 equiv). The resulting mixture was stirred at 60° C. under nitrogen atmosphere for 2 h. The reaction was quenched with water at room temperature. The resulting mixture was extracted with EtOAc (3×20 mL). The combined organic layers were washed with brine, dried over anhydrous Na₂SO₄. After filtration, the filtrate was concentrated under reduced pressure. The residue was purified by silica gel column chromatography, eluted with EA/MEOH (10:1) to afford the title compound as a yellow solid (450 mg, 71.5%). LCMS: m/z=576 [M+H]⁺

### Preparation 155: 2-(2,4-difluorophenyl)-6-methyl-4-(1-methyl-5-(3-(oxetan-3-yl)phenyl)-2-oxo-1,2-dihydropyridin-4-yl)-1-tosyl-1,6-dihydro-7H-pyrrolo[2,3-c]pyridin-7-one

To a stirred mixture of 2-chloro-6-methyl-4-(1-methyl-5-(3-(oxetan-3-yl)phenyl)-2-oxo-1,2-dihydropyridin-4-yl)-1-tosyl-1,6-dihydro-7H-pyrrolo[2,3-c]pyridin-7-one (400 mg, 0.694 mmol, 1.00 equiv) and (2,4-difluorophenyl)boronic acid (219 mg, 1.39 mmol, 2.0 equiv) in DME (10.0 mL) and H₂O (2.00 mL) at room temperature was added XPhos Pd G3 (58.8 mg, 0.069 mmol, 0.100 equiv) and K₂CO₃ (192 mg, 1.39 mmol, 2.00 equiv). The resulting mixture at 100° C. under nitrogen atmosphere was stirred for 1 h. The reaction was quenched with water at room temperature. The resulting mixture was extracted with CH₂Cl₂ (3×15 mL). The combined organic layers were washed with brine, dried over anhydrous Na₂SO₄. After filtration, the filtrate was concentrated under reduced pressure. The residue was purified by silica gel column chromatography, eluted with EA/MeOH (10:1) to afford the title compound as a grey solid (370 mg, 81.5%). LCMS: m/z=654 [M+H]⁺

### Preparation 156: 2-(2,4-difluorophenyl)-6-methyl-4-(1-methyl-5-(3-(oxetan-3-yl)phenyl)-2-oxo-1,2-dihydropyridin-4-yl)-1,6-dihydro-7H-pyrrolo[2, 3-c]pyridin-7-one

To a stirred mixture of 2-(2,4-difluorophenyl)-6-methyl-4-(1-methyl-5-(3-(oxetan-3-yl)phenyl)-2-oxo-1,2-dihydropyridin-4-yl)-1-tosyl-1,6-dihydro-7H-pyrrolo[2,3-c]pyridin-7-one (350 mg, 0.535 mmol, 1.00 equiv) in MeOH (4.0 mL) and H₂O (1.0 mL) at room temperature was added NaOH (214 mg, 5.35 mmol, 10.0 equiv). The resulting mixture was stirred at 60° C. for 1 h. The reaction was quenched with water at room temperature. The resulting mixture was extracted with CH₂Cl₂ (2×10.0 mL). The combined organic layers were washed with brine, dried over anhydrous Na₂SO₄. After filtration, the filtrate was concentrated under reduced pressure. The crude product was purified by Prep-HPLC^{BD} to afford the title compound as a white solid (120 mg, 44.7%). 1H NMR (400 MHz, DMSO-d⁶) δ 11.69 (s, 1H), 7.92-7.86 (m, 2H), 7.35-7.28 (m, 2H), 6.89-6.75 (m, 3H), 6.66-6.55 (m, 2H), 6.55 (s, 2H), 5.98 (s, 1H), 4.82-4.78 (m, 2H), 4.34-4.31 (m, 2H), 4.12-4.10 (m, 1H), 3.55 (s, 3H), 3.51 (s, 3H). LCMS^{AA}: m/z=500 [M+H]⁺.

### Example 36: 2-(2,4-difluorophenyl)-6-methyl-4-(1-methyl-5-(3-(morpholinomethyl)phenyl)-2-oxo-1,2-dihydropyridin-4-yl)-1,6-dihydro-7H-pyrrolo[2,3-c]pyridin-7-one

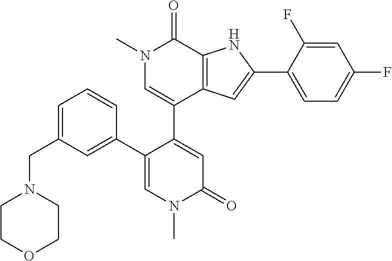 [SMILES: Cn1cc(-c2cccc(CN3CCOCC3)c2)c(-c2cn(C)c(=O)c3[nH]c(-c4ccc(F)cc4F)cc23)cc1=O]

### Preparation 157: 4-bromo-1-methyl-5-(3-(morpholinomethyl)phenylpyridin-2 (1H)-one

To a stirred mixture of 4-bromo-5-iodo-1-methylpyridin-2 (1H)-one (2.75 g, 8.76 mmol, 1.00 equiv) and 4-(3-(4,4,5,5-tetramethyl-1,3,2-dioxaborolan-2-yl)benzyl)morpholine (3.98 g, 13.1 mmol, 1.50 equiv) in DMF (40.0 mL) and H₂O (4.0 mL) at room temperature was added Na₂CO₃ (402.79 mg, 17.52 mmol, 2.00 equiv) and Pd(PPh₃)₄ (1.01 g, 0.876 mmol, 0.100 equiv) under nitrogen atmosphere. The resulting mixture was stirred at 100° C. for 3 h under nitrogen atmosphere. The resulting mixture was extracted with EtOAc (3×30 mL), dried over anhydrous Na₂SO₄. After filtration, the filtrate was concentrated under reduced pressure. The residue was purified by silica gel column chromatography, eluted with EA/MeOH (10:1) to afford the title compound as a yellow solid (1.80 g, 56.6%). LCMS: m/z=365 [M+H]⁺

### Preparation 158: 2-chloro-6-methyl-4-(1-methyl-5-(3-(morpholinomethyl)phenyl)-2-oxo-1,2-dihydropyridin-4-yl)-1-tosyl-1,6-dihydro-7H-pyrrolo[2, 3-c]pyridin-7-one

To a stirred mixture of 4-bromo-1-methyl-5-(3-(morpholinomethyl)phenyl)pyridin-2 (1H)-one (1.80 g, 4.96 mmol, 1.00 equiv) and 2-chloro-4-(5,5-dimethyl-1,3,2-dioxaborinan-2-yl)-6-methyl-1-tosyl-1,6-dihydro-7H-pyrrolo[2,3-c]pyridin-7-one (2.67 g, 5.95 mmol, 1.20 equiv) in DME (40.0 mL) and H₂O (8.0 mL) at room temperature was added Na₂CO₃ (1.05 g, 9.91 mmol, 2.00 equiv) and Pd(dppf)Cl₂CH₂Cl₂ (404 mg, 0.496 mmol, 0.100 equiv) under nitrogen atmosphere. The resulting mixture was stirred at 60° C. under nitrogen atmosphere for 3 h. The resulting mixture was extracted with EtOAc (3×30 mL), dried over anhydrous Na₂SO₄. After filtration, the filtrate was concentrated under reduced pressure. The residue was purified by silica gel column chromatography, eluted with EA/MeOH (10:1) to afford the title compound as a yellow solid (1.70 g, 55.4%). LCMS: m/z=619 [M+H]⁺.

### Preparation 159: 2-(2,4-difluorophenyl)-6-methyl-4-(1-methyl-5-(3-(morpholinomethyl)phenyl)-2-oxo-1,2-dihydropyridin-4-yl)-1-tosyl-1,6-dihydro-7H-pyrrolo[2,3-c]pyridin-7-one

To a stirred mixture of 2-chloro-6-methyl-4-(1-methyl-5-(3-(morpholinomethyl)phenyl)-2-oxo-1,2-dihydropyridin-4-yl)-1-tosyl-1,6-dihydro-7H-pyrrolo[2,3-c]pyridin-7-one (800 mg, 1.29 mmol, 1.00 equiv) and 2,4-difluorophenylboronic acid (306 mg, 1.94 mmol, 1.50 equiv) in DME (10.0 mL) and H₂O (2.0 mL) at room temperature was added Na₂CO₃ (274 mg, 2.58 mmol, 2.00 equiv) and XPhos Pd G3 (2.73 mg, 0.003 mmol, 0.100 equiv). The resulting mixture at 100° C. under nitrogen atmosphere was stirred for 2 h. The resulting mixture was extracted with CH₂Cl₂ (3×15 mL), dried over anhydrous Na₂SO₄. After filtration, the filtrate was concentrated under reduced pressure. The residue was purified by silica gel column chromatography, eluted with EA/MeOH (10:1) to afford the title compound as a yellow solid (700 mg, 77.8%). LCMS: m/z=697 [M+H]⁺

### Preparation 160: 2-(2,4-difluorophenyl)-6-methyl-4-(1-methyl-5-(3-(morpholinomethyl)phenyl)-2-oxo-1,2-dihydropyridin-4-yl)-1,6-dihydro-7H-pyrrolo[2, 3-c]pyridin-7-one

To a stirred mixture of 2-(2,4-difluorophenyl)-6-methyl-4-(1-methyl-5-(3-(morpholinomethyl)phenyl)-2-oxo-1,2-dihydropyridin-4-yl)-1-tosyl-1,6-dihydro-7H-pyrrolo[2,3-c]pyridin-7-one (700 mg, 1.01 mmol, 1.00 equiv) in MeOH (8.0 mL) and H₂O (2.0 mL) at room temperature was added NaOH (402 mg, 10.1 mmol, 10.0 equiv). The resulting mixture was stirred at 60° C. under nitrogen atmosphere for 2 h. The resulting mixture was extracted with EtOAc (3×10 mL), dried over anhydrous Na₂SO₄. After filtration, the filtrate was concentrated under reduced pressure. The crude product was purified by Prep-HPLC^{BE} to afford the title compound as a white solid (162 mg, 29.7%).

¹H NMR (400 MHz, DMSO-d⁶) δ 12.20 (s, 1H), 7.98-7.86 (m, 1H), 7.85 (s, 1H), 7.37-7.27 (m, 2H), 7.26-7.18 (m, 2H), 7.18-7.09 (m, 1H), 7.04-6.99 (m, 1H), 6.96 (d, J=1.9 Hz, 1H), 6.48 (s, 1H), 6.04 (d, J=3.3 Hz, 1H), 3.59 (s, 3H), 3.55 (s, 3H), 3.34 (m, 2H), 3.17 (m, 2H), 1.88 (m, 4H). LCMS^{L}: m/z=543 [M+H]⁺.

### Example 37: 2-(2,4-difluorophenyl)-6-methyl-4-(1-methyl-6-oxo-1,6-dihydro-[3,3′-bipyridin]-4-yl)-1,6-dihydro-7H-pyrrolo[2,3-c]pyridin-7-one

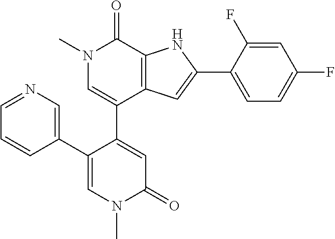 [SMILES: Cn1cc(-c2cccnc2)c(-c2cn(C)c(=O)c3[nH]c(-c4ccc(F)cc4F)cc23)cc1=O]

### Preparation 161: 4-bromo-1-methyl-[3,3′-bipyridin]-6 (1H)-one

To a stirred mixture of 4-bromo-5-iodo-1-methylpyridin-2 (1H)-one (2.00 g, 6.37 mmol, 1.00 equiv) and pyridin-3-ylboronic acid (0.939 g, 7.65 mmol, 1.20 equiv) in DMF (20 mL) and H₂O (4.0 mL) at room temperature was added Pd(PPh₃)₄ (0.736 g, 0.637 mmol, 0.100 equiv) and Na₂CO₃ (1.35 g, 12.7 mmol, 2.00 equiv). The resulting mixture at 100° C. under nitrogen atmosphere was stirred for 1 h. The resulting mixture was extracted with EtOAc (3×100 mL). The combined organic layers were washed with brine (2×100 mL), dried over anhydrous Na₂SO₄. After filtration, the filtrate was concentrated under reduced pressure. The residue was purified by RP-HPLC^{BF} to afford the title compound as a light-yellow solid (1.34 g, 79.3%). LCMS: m/z=267 [M+H]⁺.

### Preparation 162: 4-[2-chloro-6-methyl-1-(4-methylbenzenesulfonyl)-7-oxopyrrolo[2,3-c]pyridin-4-yl]-1-methyl-[3,3′-bipyridin]-6-one

To a stirred mixture of 4-bromo-1-methyl-[3,3′-bipyridin]-6 (1H)-one (1.06 g, 4.01 mmol, 1.20 equiv) and 2-chloro-4-(5,5-dimethyl-1,3,2-dioxaborinan-2-yl)-6-methyl-1-tosyl-1,6-dihydro-7H-pyrrolo[2,3-c]pyridin-7-one (1.50 g, 3.34 mmol, 1.00 equiv) in DME (15 mL) and H₂O (3 mL) at room temperature was added Pd(dppf)Cl₂·CH₂Cl₂ (272 mg, 0.334 mmol, 0.100 equiv) and Na₂CO₃ (0.708 g, 6.69 mmol, 2.00 equiv). The resulting mixture at 60° C. under nitrogen atmosphere was stirred overnight. The resulting mixture was extracted with EtOAc (3×20.0 mL). The combined organic layers were washed with brine, dried over anhydrous Na₂SO₄. After filtration, the filtrate was concentrated under reduced pressure. The residue was purified by silica gel column chromatography, eluted with MeOH/EA (10:1) to afford the title compound as a light-yellow solid (1.10 g, 63.1%). LCMS: m/z=521 [M+H]⁺

### Preparation 163: 4-[2-(2,4-difluorophenyl)-6-methyl-1-(4-methylbenzenesulfonyl)-7-oxopyrrolo[2,3-c]pyridin-4-yl]-1-methyl-[3,3′-bipyridin]-6-one

To a stirred solution of 4-[2-chloro-6-methyl-1-(4-methylbenzenesulfonyl)-7-oxopyrrolo[2,3-c]pyridin-4-yl]-1-methyl-[3,3′-bipyridin]-6-one (1.00 g, 1.91 mmol, 1.00 equiv) and 2,4-difluorophenylboronic acid (454 mg, 2.87 mmol, 1.50 equiv) in DME (15.0 mL) and H₂O (3.00 mL) at room temperature was added K₂CO₃ (530 mg, 3.84 mmol, 2.00 equiv) and XPhos Pd G3 (162 mg, 0.192 mmol, 0.100 equiv). The resulting mixture was stirred at 100° C. for 2 h under nitrogen atmosphere. The resulting mixture was extracted with EtOAc (3×15 mL). The combined organic layers were washed with brine, dried over anhydrous Na₂SO₄. After filtration, the filtrate was concentrated under reduced pressure. The residue was purified by silica gel column chromatography, eluted with EA/MeOH (5:1) to afford the title compound as a brown solid (800 mg, 41.8%). LCMS: m/z=599 [M+H]⁺

### Preparation 164: 2-(2,4-difluorophenyl)-6-methyl-4-(1-methyl-6-oxo-1,6-dihydro-[3,3′-bipyridin]-4-yl)-1,6-dihydro-7H-pyrrolo[2,3-c]pyridin-7-one

To a stirred mixture of 4-[2-(2,4-difluorophenyl)-6-methyl-1-(4-methylbenzenesulfonyl)-7-oxopyrrolo[2,3-c]pyridin-4-yl]-1-methyl-[3,3′-bipyridin]-6-one (800 mg, 1.33 mmol, 1.00 equiv) in MeOH (8.0 mL) and H₂O (2.0 mL) at room temperature was added NaOH (534 mg, 13.3 mmol, 10.0 equiv). The resulting mixture was stirred at 60° C. for 2 h. The resulting mixture was extracted with EtOAc (3×10 mL). The combined organic layers were washed with brine, dried over anhydrous Na₂SO₄. After filtration, the filtrate was concentrated under reduced pressure. The residue was purified by Prep-HPLC^{BG} to afford the title compound as a white solid (344.8 mg, 57.2%). ¹H NMR (400 MHz, DMSO-d⁶) δ 12.28 (s, 1H), 8.44-8.40 (m, 1H), 8.29-8.24 (m, 1H), 8.00-7.87 (m, 2H), 7.60-7.50 (m, 1H), 7.38-7.28 (m, 2H), 7.22-7.10 (m, 2H), 6.51 (s, 1H), 6.06 (d, J=3.3 Hz, 1H), 3.56 (s, 3H), 3.53 (s, 3H). LCMS^{A}H: m/z=445 [M+H]⁺.

### Example 38: 4-(5-(2,6-dimethylphenoxy)-1-methyl-2-oxo-1,2-dihydropyridin-4-yl)-6-methyl-2-(pyridin-2-yl)-1,6-dihydro-7H-pyrrolo[2,3-c]pyridin-7-one

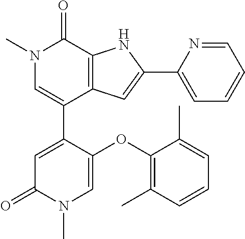 [SMILES: Cc1cccc(C)c1Oc1cn(C)c(=O)cc1-c1cn(C)c(=O)c2[nH]c(-c3ccccn3)cc12]

### Preparation 165: 4-(5-(2,6-dimethylphenoxy)-1-methyl-2-oxo-1,2-dihydropyridin-4-yl)-6-methyl-2-(pyridin-2-yl)-1-tosyl-1,6-dihydro-7H-pyrrolo[2,3-c]pyridin-7-one

To a stirred mixture of 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c]pyridin-7-one (150 mg, 0.266 mmol, 1.00 equiv) and 2-(tributylstannyl)pyridine (97.0 mg, 0.266 mmol, 1.00 equiv) in DMF (2.0 mL) at room temperature was added CuI (2.53 mg, 0.013 mmol, 0.05 equiv), LiCl (27.6 mg, 0.652 mmol, 2.45 equiv) and Pd(PPh₃)₂Cl₂ (9.33 mg, 0.013 mmol, 0.05 equiv). The resulting mixture at 100° C. under nitrogen atmosphere was stirred for 1 h. The resulting mixture was extracted with EtOAc (3×10 mL). The combined organic layers were washed with brine, dried over anhydrous Na₂SO₄. After filtration, the filtrate was concentrated under reduced pressure. The residue was purified by Prep-TLC, eluted with EA/MeOH (10:1) to afford the title product as a white solid (20 mg, 12.4%). LCMS: m/z=607 [M+H]⁺

### Preparation 166: 4-(5-(2,6-dimethylphenoxy)-1-methyl-2-oxo-1,2-dihydropyridin-4-yl)-6-methyl-2-(pyridin-2-yl)-1,6-dihydro-7H-pyrrolo[2,3-c]pyridin-7-one

To a stirred solution of 4-(5-(2,6-dimethylphenoxy)-1-methyl-2-oxo-1,2-dihydropyridin-4-yl)-6-methyl-2-(pyridin-2-yl)-1-tosyl-1,6-dihydro-7H-pyrrolo[2,3-c]pyridin-7-one (20.0 mg, 0.0330 mmol, 1.00 equiv) in MeOH (0.5 mL) and H₂O (0.1 mL) at room temperature was added NaOH (13.2 mg, 0.330 mmol, 10.0 equiv). The resulting mixture at 60° C. was stirred for 1 h. The resulting mixture was extracted with EtOAc (3×1 mL). The combined organic layers were washed with brine, dried over anhydrous Na₂SO₄. After filtration, the filtrate was concentrated under reduced pressure. The residue was purified by Prep-HPLC^{AR} to afford the title product as a white solid (2.9 mg, 12.0%). ¹H NMR (400 MHz, Methanol-d⁴) δ 8.67-8.56 (m, 1H), 7.97-7.77 (m, 2H), 7.58 (s, 1H), 7.39-7.28 (m, 1H), 7.15-7.01 (m, 4H), 6.84 (s, 1H), 6.68 (s, 1H), 3.73 (s, 3H), 3.47 (s, 3H), 2.15 (s, 6H). LCMS^{Z}: m/z 453 [M+H]⁺

### Example 39:4-(1-cyclopropyl-5-(2,6-dimethylphenoxy)-2-oxo-1,2-dihydropyridin-4-yl)-2-(2,4-difluorophenyl)-6-methyl-1,6-dihydro-7H-pyrrolo[2,3-c]pyridin-7-one

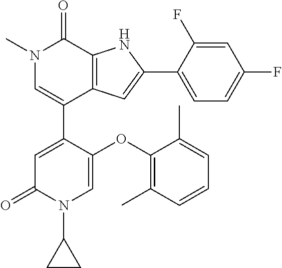 [SMILES: Cc1cccc(C)c1Oc1cn(C2CC2)c(=O)cc1-c1cn(C)c(=O)c2[nH]c(-c3ccc(F)cc3F)cc12]

### Preparation 167: 2-bromo-5-(2,6-dimethylphenoxy)pyridine

To a stirred mixture of 2-bromo-5-fluoropyridine (200 g, 1.14 mol, 1.00 equiv) and 2,6-dimethylphenol (138.9 g, 1.13 mol, 1.00 equiv) in DMSO (2.0 L) was added Cs₂CO₃ (407.3 g, 1.25 mol, 1.10 equiv) in portions at room temperature. The resulting mixture was stirred at 120° C. for 2 h. The reaction was quenched by the addition of water/ice (1.0 L) at room temperature. The resulting mixture was extracted with EtOAc (5×500 mL). The combined organic layers were washed with brine (3×500 mL), dried over anhydrous Na₂SO₄, filtered and concentrated under reduced pressure. The residue was purified by silica gel column chromatography, eluted with PE/EA (12:1) to afford the title compound as an off-white solid (248 g, 78.5%). ¹H NMR (400 MHz, DMSO-d⁶) δ 8.03 (d, J=3.1 Hz, 1H), 7.55 (d, J=8.7 Hz, 1H), 7.20-7.11 (m, 3H), 7.04 (dd, J=8.7, 3.2 Hz, 1H), 2.07 (s, 6H). LCMS: m/z=280 [M+H]⁺.

### Preparation 168: 2-bromo-5-(2,6-dimethylphenoxy)-4-iodopyridine

To a solution of 2-bromo-5-(2,6-dimethylphenoxy)pyridine (248 g, 0.89 mol, 1.00 equiv) in THF (2.5 L) at −78° C. under N₂ atmosphere was added LDA (513 mL, 1.26 mol, 2.0 M/L in THF/Hexane) dropwise. The mixture was stirred at −78° C. for 40 min. To the above mixture was added a solution of 12 (1.13 kg, 4.46 mol, 5.0 equiv) in THF (200 mL) dropwise. The mixture was stirred for another 15 min and then allowed to warm up to room temperature. The mixture at room temperature was stirred for 1 h. The reaction was quenched with aq. Na₂S₂O₃ (600 mL). The mixture was extracted with EtOAc (3×500 mL). The combined organic phases were washed with brine (500 mL), dried over anhydrous Na₂SO₄, filtered and concentrated under vacuum. The residue was purified by silica gel column chromatography, eluted with PE/EA (10:1) to afford the title compound as a white solid (260 g, 72.2%). 1H NMR (300 MHz, DMSO-d⁶) δ 8.24 (s, 1H), 7.27-7.12 (m, 4H), 2.07 (s, 6H). LCMS: m/z=406 [M+H]⁺.

### Preparation 169: 5-(2,6-dimethylphenoxy)-4-iodopyridin-2 (1H)-one

To a stirred solution of 2-bromo-5-(2,6-dimethylphenoxy)-4-iodopyridine (260 g, 0.64 mol, 1.00 equiv) in t-BuOH (2.6 L) was added KOH (361.0 g, 6.43 mol, 10.0 equiv) in portions at room temperature. The resulting mixture was stirred for 6 h at 120° C. in an autoclave. The mixture was allowed to cool down to room temperature. The reaction was quenched with water/ice at room temperature. The aqueous layer was extracted with CH₂Cl₂ (3×500 mL). The combined organic layer was concentrated under reduced pressure. The residue was purified by silica gel column chromatography, eluted with PE/EA (1:4) to afford crude product. The crude product was further purified by trituration with aq. K₂CO₃ (2.0 L, 1.0 N) overnight. After filtration, the filter cake was dried to afford the title compound as a white solid (90.4 g, 41.2%). ¹H NMR (400 MHz, DMSO-d⁶) δ 11.14 (s, 1H), 7.17-7.07 (m, 4H), 6.23 (s, 1H), 2.09 (s, 6H). LCMS: m/z=342 [M+H]⁺.

### Preparation 170: 1-cyclopropyl-5-(2,6-dimethylphenoxy)-4-iodopyridin-2 (1H)-one

A mixture of 5-(2,6-dimethylphenoxy)-4-iodo-1H-pyridin-2-one (6.00 g, 17.6 mmol, 1.00 equiv) and Na₂CO₃ (4.23 g, 39.9 mmol, 2.27 equiv) at room temperature in DCE (120 mL) was stirred for 30 min. To the above mixture was added cyclopropylboronic acid (3.32 g, 38.7 mmol, 2.20 equiv), 2′-bipyridine (2.94 g, 18.8 mmol, 1.07 equiv) and Cu(OAc)₂ (3.42 g, 18.8 mmol, 1.07 equiv). The resulting mixture at 60° C. under an 02 atmosphere was stirred for 3 h. The mixture was allowed to cool down to room temperature. The aqueous layer was extracted with CH₂Cl₂ (5×200 mL), dried over anhydrous Na₂SO₄, filtered and concentrated under reduced pressure. The residue was purified by silica gel column chromatography, eluted with PE/EA (2:1) to afford the title compound as yellow solid (4.00 g, 59.7%). LCMS: m/z=382 [M+H⁺]

### Preparation 171: 2-chloro-4-(1-cyclopropyl-5-(2,6-dimethylphenoxy)-2-oxo-1,2-dihydropyridin-4-yl)-6-methyl-1-tosyl-1,6-dihydro-7H-pyrrolo[2, 3-c]pyridin-7-one

To a stirred mixture of 2-chloro-6-methyl-1-(4-methylbenzenesulfonyl)-4-(4,4,5,5-tetramethyl-1,3,2-dioxaborolan-2-yl)pyrrolo[2,3-c]pyridin-7-one (1.80 g, 3.89 mmol, 1.00 equiv) and 1-cyclopropyl-5-(2,6-dimethylphenoxy)-4-iodopyridin-2-one (2.22 g, 5.84 mmol, 1.50 equiv) in DME (20 mL) and H₂O (4.0 mL) at room temperature was added Na₂CO₃ (820 mg, 7.78 mmol, 2.00 equiv) and Pd(dppf)Cl₂·CH₂Cl₂ (0.320 g, 0.389 mmol, 0.100 equiv). The resulting mixture at 60° C. under nitrogen atmosphere was stirred overnight. The resulting mixture was extracted with EtOAc (3×50 mL), dried over anhydrous Na₂SO₄, filtered and concentrated under reduced pressure. The residue was purified by silica gel column chromatography, eluted with EtOAc/MeOH (10:1) to afford the title compound (900 mg, 39.2%) as a yellow solid. LCMS: m/z=590 [M+H⁺]

### Preparation 172: 4-(1-cyclopropyl-5-(2,6-dimethylphenoxy)-2-oxo-1,2-dihydropyridin-4-yl)-2-(2,4-difluorophenyl)-6-methyl-1-tosyl-1,6-dihydro-7H-pyrrolo[2, 3-c]pyridin-7-one

To a stirred mixture of 2-chloro-4-(1-cyclopropyl-5-(2,6-dimethylphenoxy)-2-oxo-1,2-dihydropyridin-4-yl)-6-methyl-1-tosyl-1,6-dihydro-7H-pyrrolo[2,3-c]pyridin-7-one (250 mg, 0.424 mmol, 1.00 equiv) and 2,4-difluorophenylboronic acid (133 mg, 0.848 mmol, 2.00 equiv) in DME (5 mL) and H₂O (1 mL) at room temperature was added K₂CO₃ (117 mg, 0.848 mmol, 2.00 equiv) and XPhos Pd G3 (133 mg, 0.848 mmol, 2.00 equiv). The resulting mixture at 90° C. under nitrogen atmosphere was stirred for 6 h. The reaction was quenched with water. The resulting mixture was extracted with EtOAc (3×5 mL). The combined organic layers were washed with brine, dried over anhydrous Na₂SO₄. After filtration, the filtrate was concentrated under reduced pressure. The crude product was used in the next step directly without further purification. This resulted in the title compound as a brown solid (239 mg, crude). LCMS: m/z=668 [M+H⁺]

### Preparation 173: 4-(1-cyclopropyl-5-(2,6-dimethylphenoxy)-2-oxo-1,2-dihydropyridin-4-yl)-2-(2,4-difluorophenyl)-6-methyl-1,6-dihydro-7H-pyrrolo[2,3-c]pyridin-7-one

To a stirred mixture of 4-(1-cyclopropyl-5-(2,6-dimethylphenoxy)-2-oxo-1,2-dihydropyridin-4-yl)-2-(2,4-difluorophenyl)-6-methyl-1-tosyl-1,6-dihydro-7H-pyrrolo[2,3-c]pyridin-7-one (220 mg, 0.330 mmol, 1.00 equiv) in MeOH (3.00 mL) and H₂O (0.30 mL) at room temperature was added NaOH (131 mg, 3.29 mmol, 10.0 equiv). The resulting mixture at 60° C. was stirred for 3 h. The resulting mixture was extracted with EtOAc (3×2.00 mL). The combined organic layers were washed with brine (3×10 mL), dried over anhydrous Na₂SO₄. After filtration, the filtrate was concentrated under reduced pressure. The residue was purified by Prep-HPLC^{BI} to afford the title compound as a white solid (45.0 mg, 26.2%). ¹H NMR (400 MHz, DMSO-d⁶) δ 12.50 (s, 1H), 8.20-8.05 (m, 1H), 7.54 (s, 1H), 7.45-7.36 (m, 1H), 7.26-7.17 (m, 1H), 7.12 (d, J=7.3 Hz, 2H), 7.09-7.03 (m, 1H), 6.77 (d, J=3.4 Hz, 1H), 6.52 (s, 1H), 6.26 (s, 1H), 3.61 (s, 3H), 3.23-3.11 (m, 1H), 2.08 (s, 6H), 1.01-0.83 (m, 2H), 0.65-0.54 (m, 2H). LCMS^{AF}: m/z=514 [M+H]⁺

### Example 40: 4-(1-cyclopropyl-5-(2,6-dimethylphenoxy)-2-oxo-1,2-dihydropyridin-4-yl)-2-(2-fluoro-3-methoxyphenyl)-6-methyl-1,6-dihydro-7H-pyrrolo[2,3-c]pyridin-7-one

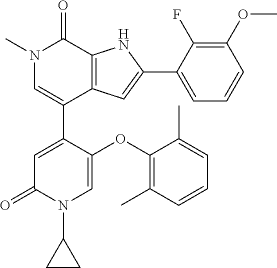

### Preparation 174: 4-(1-cyclopropyl-5-(2,6-dimethylphenoxy)-2-oxo-1,2-dihydropyridin-4-yl)-2-(2-fluoro-3-methoxyphenyl)-6-methyl-1-tosyl-1,6-dihydro-7H-pyrrolo[2,3-c]pyridin-7-one

To a mixture of 2-chloro-4-(1-cyclopropyl-5-(2,6-dimethylphenoxy)-2-oxo-1,2-dihydropyridin-4-yl)-6-methyl-1-tosyl-1,6-dihydro-7H-pyrrolo[2,3-c]pyridin-7-one (340 mg, 0.576 mmol, 1.00 equiv) and 2-fluoro-3-methoxyphenylboronic acid (196 mg, 1.15 mmol, 2.00 equiv) in DME (8.0 mL) and H₂O (2.0 mL) at room temperature was added XPhos Pd G3 (48.8 mg, 0.0580 mmol, 0.100 equiv) and K₂CO₃ (159 mg, 1.15 mmol, 2.00 equiv). The resulting mixture at 100° C. under nitrogen atmosphere was stirred overnight. The reaction was quenched with water at room temperature. The resulting mixture was extracted with EtOAc (5×10.0 mL). The combined organic layers were washed with brine, dried over anhydrous Na₂SO₄. After filtration, the filtrate was concentrated under reduced pressure to afford the title compound as a yellow crude solid (410 mg). LCMS: m/z=680 [M+H]⁺.

### Preparation 175: 4-(1-cyclopropyl-5-(2,6-dimethylphenoxy)-2-oxo-1,2-dihydropyridin-4-yl)-2-(2-fluoro-3-methoxyphenyl)-6-methyl-1,6-dihydro-7H-pyrrolo[2,3-c]pyridin-7-one

To a mixture of 4-(1-cyclopropyl-5-(2,6-dimethylphenoxy)-2-oxo-1,2-dihydropyridin-4-yl)-2-(2-fluoro-3-methoxyphenyl)-6-methyl-1-tosyl-1,6-dihydro-7H-pyrrolo[2,3-c]pyridin-7-one (410 mg, 0.603 mmol, 1.00 equiv) in MeOH (6.0 mL) and H₂O (1.5 mL) at room temperature was added NaOH (241 mg, 6.03 mmol, 10.0 equiv). The resulting mixture was stirred at 60° C. for 1 h. The reaction was quenched with water at room temperature. The resulting mixture was extracted with EtOAc (3×10.0 mL). The combined organic layers were washed with brine, dried over anhydrous Na₂SO₄. After filtration, the filtrate was concentrated under reduced pressure. The crude product was purified by Prep-HPLC^{BJ} to afford the title compound as a white solid (31.8 mg, 9.8%). 1H NMR (400 MHz, DMSO-d⁶) δ 12.45 (s, 1H), 7.61 (t, J=7.0 Hz, 1H), 7.55 (s, 1H), 7.25-7.04 (m, 5H), 6.81 (s, 1H), 6.53 (s, 1H), 6.26 (s, 1H), 3.88 (s, 3H), 3.62 (s, 3H), 3.22-3.12 (m, 1H), 2.10 (s, 6H), 0.98-0.87 (m, 2H), 0.59 (d, J=2.6 Hz, 2H). LCMS^{Q}: m/z=526 [M+H]⁺

### Example 41: 4-(5-(2,6-dimethylphenoxy)-1-methyl-2-oxo-1,2-dihydropyridin-4-yl)-6-methyl-2-(1-propyl-1H-pyrazol-4-yl)-1,6-dihydro-7H-pyrrolo[2,3-c]pyridin-7-one

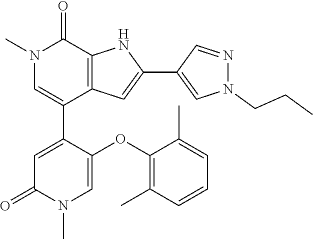 [SMILES: CCCn1cc(-c2cc3c(-c4cc(=O)n(C)cc4Oc4c(C)cccc4C)cn(C)c(=O)c3[nH]2)cn1]

### Preparation 176: 4-(5-(2,6-dimethylphenoxy)-1-methyl-2-oxo-1,2-dihydropyridin-4-yl)-6-methyl-2-(1-propyl-1H-pyrazol-4-yl)-1,6-dihydro-7H-pyrrolo[2,3-c]pyridin-7-one

Following the procedure described in preparation 70, 2-chloro-4-(5-(2,6-dimethylphenoxy)-1-methyl-2-oxo-1,2-dihydropyridin-4-yl)-6-methyl-1,6-dihydro-7H-pyrrolo[2,3-c]pyridin-7-one (0.20 g, 0.48 mmol) and 1-propyl-4-(4,4,5,5-tetramethyl-1,3,2-dioxaborolan-2-yl)-1H-pyrazole (0.461 g, 1.95 mmol) was reacted to give title compound (0.067 g, 28.36%) as an off white solid. ¹H NMR: (400 MHz, DMSO) δ 12.26 (s, 1H), 8.29 (s, 1H), 8.01 (s, 1H), 7.48 (s, 1H), 7.11 (d, J=7.2 Hz, 2H), 7.05 (m, 1H), 6.67 (s, 1H), 6.54 (d, J=9.2 Hz, 2H), 4.07 (t, J=6.8 Hz, 2H), 3.58 (s, 3H), 3.34 (s, 3H), 2.09 (s, 6H), 1.80 (q, J=7.2 Hz, 2H), 0.85 (t, J=7.6 Hz, 3H). LCMS^{XX}: m/z=484 [M+1H]⁺

### Example 42: 2-(2-fluorophenyl)-6-methyl-4-(1-methyl-2-oxo-5-phenyl-1,2-dihydropyridin-4-yl)-1,6-dihydro-7H-pyrrolo[2,3-c]pyridin-7-one

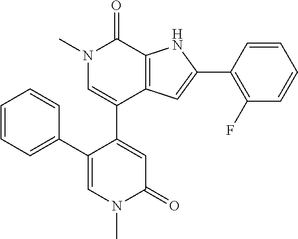 [SMILES: Cn1cc(-c2ccccc2)c(-c2cn(C)c(=O)c3[nH]c(-c4ccccc4F)cc23)cc1=O]

### Preparation 177: 2-(2-fluorophenyl)-6-methyl-4-(1-methyl-2-oxo-5-phenyl-1,2-dihydropyridin-4-yl)-1,6-dihydro-7H-pyrrolo[2,3-c]pyridin-7-one

To a solution of 2-chloro-6-methyl-4-(1-methyl-2-oxo-5-phenyl-1,2-dihydropyridin-4-yl)-1,6-dihydro-7H-pyrrolo[2,3-c]pyridin-7-one (150 mg, 0.409 mmol, 1.00 equiv) and (2-fluorophenyl) boronic acid (100 mg, 0.715 mmol, 1.74 equiv) in DME (2.0 mL) and H₂O (0.4 mL) was added K₂CO₃ (125 mg, 0.904 mmol, 2.21 equiv) and XPhos Pd G3 (75.0 mg, 0.089 mmol, 0.22 equiv). The mixture was stirred at 80° C. for 2 h under nitrogen atmosphere. The reaction was quenched by the addition of water. The aqueous layer was extracted with EtOAc (6×50 mL), filtered and concentrated under reduced pressure. The crude product was purified by Prep-HPLC^{BM} to afford the title compound as a white solid (82.6 mg, 47.0%).

¹H NMR (400 MHz, CDCl₃) δ 10.45 (s, 1H), 7.69 (t, J=7.7 Hz, 1H), 7.42 (s, 1H), 7.32-7.29 (m, 1H), 7.24-7.17 (m, 5H), 7.19-7.10 (m, 2H), 6.85 (s, 1H), 6.61-6.59 (m, 2H), 3.69 (s, 3H), 3.50 (s, 3H). LCMS^{X} (ESI, m/z): 426.0 [M+H]⁺.

### Example 43: 2-(2,4-difluorophenyl)-6-methyl-4-(1-methyl-2-oxo-5-phenyl-1,2-dihydropyridin-4-yl)-1,6-dihydro-7H-pyrrolo[2,3-c]pyridin-7-one

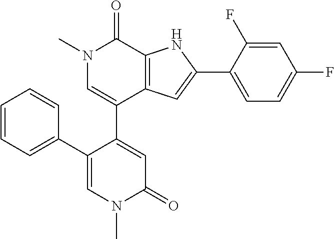 [SMILES: Cn1cc(-c2ccccc2)c(-c2cn(C)c(=O)c3[nH]c(-c4ccc(F)cc4F)cc23)cc1=O]

### Preparation 178:2-(2,4-difluorophenyl)-6-methyl-4-(1-methyl-2-oxo-5-phenyl-1,2-dihydropyridin-4-yl)-1,6-dihydro-7H-pyrrolo[2,3-c]pyridin-7-one

To a mixture of 2-chloro-6-methyl-4-(1-methyl-2-oxo-5-phenyl-1,2-dihydropyridin-4-yl)-1,6-dihydro-7H-pyrrolo[2,3-c]pyridin-7-one (2.00 g, 5.47 mmol, 1.00 equiv) and (2,4-difluorophenyl) boronic acid (1.73 g, 10.9 mmol, 2.00 equiv) in DME (50.0 mL) and H₂O (10.0 mL) was added Na₂CO₃ (1.16 g, 10.9 mmol, 2.00 equiv) and XPhos Pd G3 (463 mg, 0.547 mmol, 0.100 equiv). The resulting mixture was stirred at 60° C. under nitrogen atmosphere for 2 h. The reaction was quenched with water at room temperature. The resulting mixture was extracted with EtOAc (4×50.0 mL). The combined organic layers were washed with brine (2×10.0 mL), dried over anhydrous Na₂SO₄, filtered and concentrated under reduced pressure. The crude product was purified by Prep-HPLC^{BN} to afford the title compound as a white solid (791.2 mg, 32.6%).

¹H NMR (400 MHz, DMSO-d⁶) δ 12.24 (s, 1H), 7.96-7.90 (m, 1H), 7.85 (s, 1H), 7.30-7.29 (m, 1H), 7.22-7.07 (m, 7H), 6.14 (s, 1H), 6.13 (s, 1H), 3.55 (s, 3H), 3.49 (s, 3H). LCMS^{H} (ESI, m/z): 443.9 [M+H]⁺.

### Example 44: 6-methyl-4-(1-methyl-2-oxo-5-phenyl-1,2-dihydropyridin-4-yl)-2-(4-(oxetan-3-yl) phenyl)-1,6-dihydro-7H-pyrrolo[2,3-c]pyridin-7-one

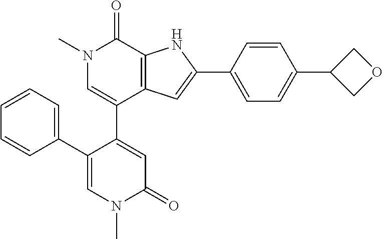 [SMILES: Cn1cc(-c2ccccc2)c(-c2cn(C)c(=O)c3[nH]c(-c4ccc(C5COC5)cc4)cc23)cc1=O]

### Preparation 179: 6-methyl-4-(1-methyl-2-oxo-5-phenyl-1,2-dihydropyridin-4-yl)-2-(4-(oxetan-3-yl) phenyl)-1,6-dihydro-7H-pyrrolo[2,3-c]pyridin-7-one

To a mixture of 2-chloro-6-methyl-4-(1-methyl-2-oxo-5-phenyl-1,2-dihydropyridin-4-yl)-1,6-dihydro-7H-pyrrolo[2,3-c]pyridin-7-one (150 mg, 0.410 mmol, 1.00 equiv) and (4-(oxetan-3-yl)phenyl)boronic acid (146 mg, 0.820 mmol, 2.00 equiv) in DME (15.0 mL) and H₂O (3.00 mL) were added XPhos Pd G3 (34.7 mg, 0.041 mmol, 0.100 equiv) and K₂CO₃ (113 mg, 0.820 mmol, 2.00 equiv). The resulting mixture was stirred at 80° C. under nitrogen atmosphere overnight. The reaction was quenched with water. The resulting mixture was extracted with EtOAc (4×50.0 mL). The combined organic layers were washed with brine (2×10.0 mL), dried over anhydrous Na₂SO₄, filtered and concentrated under reduced pressure. The crude product was purified by Prep-HPLC^{BO} to afford the title compound as a white solid (60 mg, 31.6%).

¹H NMR (400 MHz, DMSO-d⁶) δ 12.22 (s, 1H), 7.87 (s, 1H), 7.78-7.76 (m, 2H), 7.40-7.38 (m, 2H), 7.24-7.16 (m, 5H), 7.10-7.07 (m, 1H), 6.49 (s, 1H), 6.21 (s, 1H), 4.95-4.91 (m, 2H), 4.63-4.60 (m, 2H), 4.26-4.22 (m, 1H), 3.56 (s, 3H), 3.47 (s, 3H). LCMS^{H} (ESI, m/z): 464.0 [M+H]⁺

### Example 45: 6-methyl-4-(1-methyl-2-oxo-5-phenyl-1,2-dihydropyridin-4-yl)-2-(4-(methylsulfonyl)phenyl)-1,6-dihydro-7H-pyrrolo[2,3-c]pyridin-7-one

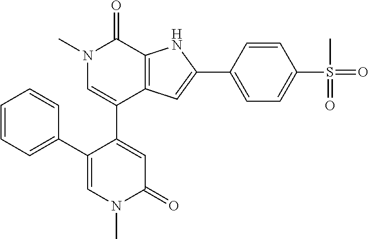 [SMILES: Cn1cc(-c2ccccc2)c(-c2cn(C)c(=O)c3[nH]c(-c4ccc(S(C)(=O)=O)cc4)cc23)cc1=O]

### Preparation 180: 6-methyl-4-(1-methyl-2-oxo-5-phenyl-1,2-dihydropyridin-4-yl)-2-(4-(methylsulfonyl)phenyl)-1,6-dihydro-7H-pyrrolo[2, 3-c]pyridin-7-one

To a stirred mixture of 2-chloro-6-methyl-4-(1-methyl-2-oxo-5-phenyl-1,2-dihydropyridin-4-yl)-1,6-dihydro-7H-pyrrolo[2,3-c]pyridin-7-one (150 mg, 0.410 mmol, 1.00 equiv) and (4-(methylsulfonyl)phenyl) boronic acid (164 mg, 0.820 mmol, 2.00 equiv) in DME (10.0 mL) and H₂O (15.0 mL) were added K₂CO₃ (113 mg, 0.820 mmol, 2.00 equiv) and XPhos Pd G3 (34.7 mg, 0.041 mmol, 0.100 equiv). The resulting mixture was stirred at 80° C. under nitrogen atmosphere for 2 h. The reaction was quenched with water at room temperature. The resulting mixture was extracted with EtOAc (4×30 mL). The combined organic layers were washed with brine (2×10 mL), dried over anhydrous Na₂SO₄. After filtration, the filtrate was concentrated under reduced pressure. The crude product was purified by Prep-HPLC^{BP} to afford the title compound as a yellow solid (22.0 mg, 11.1%).

¹H NMR (400 MHz, CDCl₃) δ 11.95 (s, 1H), 8.01-7.96 (m, 1H), 7.96-7.94 (m, 2H), 7.44 (s, 1H), 7.23-7.21 (m, 3H), 7.13-7.11 (m, 2H), 6.87 (s, 1H), 6.63 (s, 2H), 3.69 (s, 3H), 3.52 (s, 3H), 3.09 (s, 3H). LCMS^{Y} (ESI, m/z): 486.1 [M+H]⁺

### Example 46: 2-(4-(methoxymethyl)phenyl)-6-methyl-4-(1-methyl-2-oxo-5-phenyl-1,2-dihydropyridin-4-yl)-1,6-dihydro-7H-pyrrolo[2,3-c]pyridin-7-one

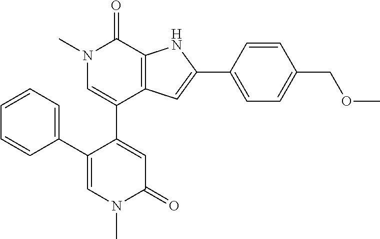 [SMILES: COCc1ccc(-c2cc3c(-c4cc(=O)n(C)cc4-c4ccccc4)cn(C)c(=O)c3[nH]2)cc1]

### Preparation 181: 2-(4-(methoxymethyl)phenyl)-4,4,5,5-tetramethyl-1,3,2-dioxaborolane

To a stirred mixture of 1-bromo-4-(methoxymethyl)benzene (1.00 g, 4.97 mmol, 1.00 equiv) and bis(pinacolato)diboron (7.58 g, 29.8 mmol, 6.00 equiv) in THF (20.0 mL) were added Pd(dppf)Cl₂·CH₂Cl₂ (405 mg, 0.497 mmol, 0.100 equiv) and KOAc (1.95 g, 19.9 mmol, 4.00 equiv). The resulting mixture was stirred at 60° C. under nitrogen atmosphere overnight. The reaction was quenched with water at room temperature. The resulting mixture was extracted with EtOAc (4×20.0 mL). The combined organic layers were washed with brine (2×10.0 mL), dried over anhydrous Na₂SO₄. After filtration, the filtrate was concentrated under reduced pressure. The product (390 mg, crude) was used in the next step directly without further purification. LCMS (ESI, m/z): 249.1 [M+H]⁺

### Preparation 182: 2-(4-(methoxymethyl)phenyl)-6-methyl-4-(1-methyl-2-oxo-5-phenyl-1,2-dihydropyridin-4-yl)-1,6-dihydro-7H-pyrrolo[2, 3-c]pyridin-7-one

To a stirred mixture of 2-(4-(methoxymethyl)phenyl)-4,4,5,5-tetramethyl-1,3,2-dioxaborolane (150 mg, 0.410 mmol, 1.00 equiv) and 2-chloro-6-methyl-4-(1-methyl-2-oxo-5-phenyl-1,2-dihydropyridin-4-yl)-1,6-dihydro-7H-pyrrolo[2,3-c]pyridin-7-one (390 mg, crude) in DME (5.00 mL) and H₂O (1.00 mL) were added XPhos Pd G3 (34.7 mg, 0.041 mmol, 0.100 equiv) and K₂CO₃ (113 mg, 0.820 mmol, 2.00 equiv). The resulting mixture was stirred at 80° C. under nitrogen atmosphere overnight. The reaction was quenched with water at room temperature. The resulting mixture was extracted with EtOAc (4×50.0 mL). The combined organic layers were washed with brine (2×10.0 mL), dried over anhydrous Na₂SO₄. After filtration, the filtrate was concentrated under reduced pressure. The crude product was purified by Prep-HPLC^{BQ} to afford the title compound as a white solid (30.6 mg, 16.5%).

¹H NMR (400 MHz, DMSO-d⁶) δ 12.22 (s, 1H), 7.87 (s, 1H), 7.76-7.74 (m, 2H), 7.30-7.28 (m, 2H), 7.23-7.17 (m, 5H), 7.10-7.07 (m, 1H), 6.49 (s, 1H), 6.22 (s, 1H), 4.40 (s, 2H), 3.56 (s, 3H), 3.47 (s, 3H), 3.29 (s, 3H). LCMS^{E} (ESI, m/z): 452.0 [M+H]⁺

### Example 47: 4-(1-cyclopropyl-2-oxo-5-phenyl-1,2-dihydropyridin-4-yl)-2-(2-fluorophenyl)-6-methyl-1,6-dihydro-7H-pyrrolo[2,3-c]pyridin-7-one

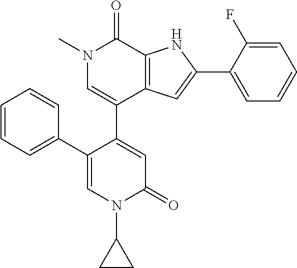 [SMILES: Cn1cc(-c2cc(=O)n(C3CC3)cc2-c2ccccc2)c2cc(-c3ccccc3F)[nH]c2c1=O]

### Preparation 183:2-chloro-4-(1-cyclopropyl-2-oxo-5-phenyl-1,2-dihydropyridin-4-yl)-6-methyl-1-tosyl-1,6-dihydro-7H-pyrrolo[2,3-c]pyridin-7-one

To a stirred mixture of 4-bromo-1-cyclopropyl-5-phenylpyridin-2 (1H)-one (2.98 g, 10.2 mmol, 1.50 equiv) and 2-chloro-4-(5,5-dimethyl-1,3,2-dioxaborinan-2-yl)-6-methyl-1-tosyl-1,6-dihydro-7H-pyrrolo[2,3-c]pyridin-7-one (3.07 g, 6.84 mmol, 1.00 equiv) in DME (60.0 mL) and H₂O (0.10 mL) at room temperature under nitrogen atmosphere were added Na₂CO₃ (1.45 g, 13.6 mmol, 2.00 equiv) and Pd(dppf)Cl₂·CH₂Cl₂ (560 mg, 0.680 mmol, 0.10 equiv). The resulting mixture was extracted with EtOAc (3×60 mL). The combined organic layers were washed with brine (3×50 mL), dried over anhydrous Na₂SO₄. After filtration, the filtrate was concentrated under reduced pressure. The residue was purified by silica gel column chromatography, eluted with EA/MeOH (10:1) to afford the title compound as a yellow solid (2.33 g, 62.4%). LCMS (ESI, m/z): 547.0 [M+H]⁺

### Preparation 184: 4-(1-cyclopropyl-2-oxo-5-phenyl-1,2-dihydropyridin-4-yl)-2-(2-fluorophenyl)-6-methyl-1-tosyl-1,6-dihydro-7H-pyrrolo[2,3-c]pyridin-7-one

To a solution of 2-chloro-4-(1-cyclopropyl-2-oxo-5-phenyl-1,2-dihydropyridin-4-yl)-6-methyl-1-tosyl-1,6-dihydro-7H-pyrrolo[2,3-c]pyridin-7-one (150 mg, 0.27 mmol, 1.00 equiv) and 2-fluorophenylboronic acid (76.9 mg, 0.55 mmol, 2.00 equiv) in DME (2.00 mL) and H₂O (0.50 mL) were added XPhos Pd G3 (46.4 mg, 0.05 mmol, 0.10 equiv) and K₂CO₃ (75.8 mg, 0.54 mmol, 2.00 equiv). The mixture was stirred at 100° C. under nitrogen atmosphere for 2 h. The resulting mixture was filtered, the filter cake was washed with DCM (50 mL). The filtrate was concentrated under reduced pressure. This resulted the title compound as a yellow solid (370 mg, crude). The crude product was used in the next step directly without further purification. LCMS (ESI, m/z): 606.6 [M+H]⁺

### Preparation 185: 4-(1-cyclopropyl-2-oxo-5-phenyl-1,2-dihydropyridin-4-yl)-2-(2-fluorophenyl)-6-methyl-1,6-dihydro-7H-pyrrolo[2,3-c]pyridin-7-one

To a solution of 4-(1-cyclopropyl-2-oxo-5-phenyl-1,2-dihydropyridin-4-yl)-2-(2-fluorophenyl)-6-methyl-1-tosyl-1,6-dihydro-7H-pyrrolo[2,3-c]pyridin-7-one (360 mg, 0.59 mmol, 1.00 equiv) in MeOH (4.00 mL) and H₂O (1.00 mL) was added NaOH (237 mg, 5.94 mmol, 10.0 equiv). The reaction was stirred at 60° C. for 1 h. The resulting mixture was extracted with EtOAc (3×10 ml). The combined organic phases were dried over anhydrous Na₂SO₄. After filtration, the filtrate was concentrated under reduced pressure. The residue was purified by silica gel column chromatography, eluted with PE/EA (1:1) to afford the title compound as a white solid (18.2 mg, 6.78%).

¹H NMR (400 MHz, CDCl₃) δ 10.29 (s, 1H), 7.74-7.65 (m, 1H), 7.36 (s, 1H), 7.33-7.27 (m, 1H), 7.30-7.16 (m, 4H), 7.24-7.10 (m, 6H), 6.85 (s, 1H), 6.61-6.55 (m, 2H), 3.48-3.44 (m, 4H), 1.23-1.18 (m, 2H), 1.02-1.00 (m, 2H). LCMS^{L} (ESI, m/z): 452.5 [M+H]⁺

### Example 48: 4-(1-cyclopropyl-2-oxo-5-phenyl-1,2-dihydropyridin-4-yl)-2-(2,3-difluorophenyl)-6-methyl-1,6-dihydro-7H-pyrrolo[2,3-c]pyridin-7-one

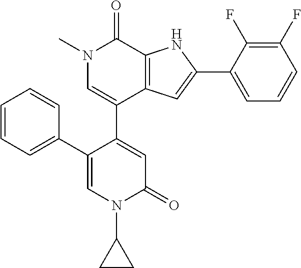 [SMILES: Cn1cc(-c2cc(=O)n(C3CC3)cc2-c2ccccc2)c2cc(-c3cccc(F)c3F)[nH]c2c1=O]

### Preparation 186: 4-(1-cyclopropyl-2-oxo-5-phenyl-1,2-dihydropyridin-4-yl)-2-(2,3-difluorophenyl)-6-methyl-1-tosyl-1,6-dihydro-7H-pyrrolo[2,3-c]pyridin-7-one

To a solution of 2-chloro-4-(1-cyclopropyl-2-oxo-5-phenyl-1,2-dihydropyridin-4-yl)-6-methyl-1-tosyl-1,6-dihydro-7H-pyrrolo[2,3-c]pyridin-7-one (900 mg, 1.65 mmol, 1.00 equiv) and (2,3-difluorophenyl)boronic acid (520 mg, 3.30 mmol, 2.00 equiv) in DME (60.0 mL) and H₂O (12.0 mL) under nitrogen atmosphere was added with XPhos Pd G3 (140 mg, 0.165 mmol, 0.10 equiv) and K₂CO₃ (456 mg, 3.30 mmol, 2.00 equiv). The mixture was stirred at 100° C. overnight. The reaction was quenched with water at room temperature. The resulting mixture was extracted with EtOAc (5×100 mL). The combined organic layers were washed with brine (3×100 mL), dried over anhydrous Na₂SO₄. After filtration, the filtrate was concentrated under reduced pressure. This resulted in the title compound as a black oil (1.10 g, crude). The crude product was used in the next step directly without further purification. LCMS (ESI, m/z): 624.7 [M+H]⁺

### Preparation 187: 4-(1-cyclopropyl-2-oxo-5-phenyl-1,2-dihydropyridin-4-yl)-2-(2,3-difluorophenyl)-6-methyl-1,6-dihydro-7H-pyrrolo[2, 3-c]pyridin-7-one

To a solution of 4-(1-cyclopropyl-2-oxo-5-phenyl-1,2-dihydropyridin-4-yl)-2-(2,3-difluorophenyl)-6-methyl-1-tosyl-1,6-dihydro-7H-pyrrolo[2,3-c]pyridin-7-one (1.02 g, 1.64 mmol, 1.00 equiv) in MeOH (12.0 mL) and H₂O (3.0 mL) was added NaOH (654 mg, 16.4 mmol, 10.0 equiv). The mixture was stirred at 60° C. for 1 h. The reaction was quenched with water at room temperature. The resulting mixture was extracted with CH₂Cl₂ (5×20 mL). The combined organic layers were washed with brine (3×20 mL), dried over anhydrous Na₂SO₄. After filtration, the filtrate was concentrated under reduced pressure. The crude product was purified by Prep-HPLC^{BR} to afford the title compound as a white solid (37.7 mg, 4.8%).

¹H NMR (400 MHz, DMSO-d⁶) δ 12.32 (s, 1H), 7.70 (t, J=7.3 Hz, 1H), 7.52 (s, 1H), 7.36 (t, J=8.5 Hz, 1H), 7.26-7.14 (m, 6H), 7.08 (t, J=7.0 Hz, 1H), 6.48 (s, 1H), 6.19 (s, 1H), 3.49 (s, 3H), 3.46-3.39 (m, 1H), 1.04-1.01 (m, 4H). LCMS^{H} (ESI, m/z): 470.0 [M+H]⁺

### Example 49: 4-(1-cyclopropyl-2-oxo-5-phenyl-1,2-dihydropyridin-4-yl)-2-(2,4-difluorophenyl)-6-methyl-1,6-dihydro-7H-pyrrolo[2,3-c]pyridin-7-one

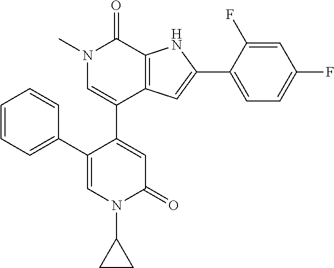 [SMILES: Cn1cc(-c2cc(=O)n(C3CC3)cc2-c2ccccc2)c2cc(-c3ccc(F)cc3F)[nH]c2c1=O]

### Preparation 188: 4-(1-cyclopropyl-2-oxo-5-phenyl-1,2-dihydropyridin-4-yl)-2-(2,4-difluorophenyl)-6-methyl-1-tosyl-1,6-dihydro-7H-pyrrolo[2,3-c]pyridin-7-one

To a solution of 2-chloro-4-(1-cyclopropyl-2-oxo-5-phenyl-1,2-dihydropyridin-4-yl)-6-methyl-1-tosyl-1,6-dihydro-7H-pyrrolo[2,3-c]pyridin-7-one (580 mg, 1.06 mmol, 1.00 equiv) and (2,4-difluorophenyl)boronic acid (335 mg, 2.12 mmol, 2.00 equiv) in DME (10.0 mL) and H₂O (2.0 mL) was added XPhos Pd G3 (89.9 mg, 0.106 mmol, 0.10 equiv) and K₂CO₃ (294 mg, 2.12 mmol, 2.00 equiv). The mixture was stirred at 100° C. under nitrogen atmosphere for 2 h. The reaction was quenched with water at room temperature. The resulting mixture was extracted with EtOAc (5×20 mL). The combined organic layers were washed with brine (3×20 mL), dried over anhydrous Na₂SO₄. After filtration, the filtrate was concentrated under reduced pressure. This resulted in the title compound as a black oil (850 mg, crude). The crude product was used in the next step directly without further purification. LCMS (ESI, m/z): 624.7 [M+H]⁺

### Preparation 189: 4-(1-cyclopropyl-2-oxo-5-phenyl-1,2-dihydropyridin-4-yl)-2-(2,4-difluorophenyl)-6-methyl-1,6-dihydro-7H-pyrrolo[2,3-c]pyridin-7-one

To a solution of 4-(1-cyclopropyl-2-oxo-5-phenyl-1,2-dihydropyridin-4-yl)-2-(2,4-difluorophenyl)-6-methyl-1-tosyl-1,6-dihydro-7H-pyrrolo[2,3-c]pyridin-7-one (820 mg, 1.32 mmol, 1.00 equiv) in CH₃OH (10.0 mL) and H₂O (2.0 mL) was added NaOH (526 mg, 13.2 mmol, 10.0 equiv). The mixture was stirred at 60° C. for 1 h. The reaction was quenched with water at room temperature. The resulting mixture was extracted with CH₂Cl₂ (5×20 mL). The combined organic layers were washed with brine (3×20 mL), dried over anhydrous Na₂SO₄. After filtration, the filtrate was concentrated under reduced pressure. The crude product was purified by Prep-HPLC^{BS} to afford the title compound as a white solid (37.4 mg, 5.8%).

¹H NMR (400 MHz, CDCl₃) δ 10.99 (s. 1H), 7.74-7.72 (m, 1H), 7.43 (s, 1H), 7.21-7.19 (m, 3H), 7.08-7.00 (m, 2H), 7.03-6.89 (m, 2H), 6.83 (s, 1H), 6.67 (s, 1H), 6.58 (s, 1H), 3.55 (s, 3H), 3.51-3.44 (m, 1H), 1.28-1.20 (m, 2H), 1.01-1.00 (m, 2H). LCMS^{AP} (ESI, m/z): 470.0 [M+H]⁺

### Example 50: 4-(1-cyclopropyl-2-oxo-5-phenyl-1,2-dihydropyridin-4-yl)-2-(2-fluoro-3-methoxyphenyl)-6-methyl-1,6-dihydro-7H-pyrrolo[2,3-c]pyridin-7-one

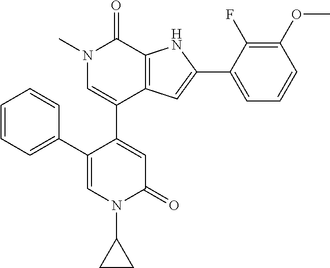 [SMILES: COc1cccc(-c2cc3c(-c4cc(=O)n(C5CC5)cc4-c4ccccc4)cn(C)c(=O)c3[nH]2)c1F]

### Preparation 190: 4-(1-cyclopropyl-2-oxo-5-phenyl-1,2-dihydropyridin-4-yl)-2-(2-fluoro-3-methoxyphenyl)-6-methyl-1-tosyl-1,6-dihydro-7H-pyrrolo[2,3-c]pyridin-7-one

To a solution of 2-chloro-4-(1-cyclopropyl-2-oxo-5-phenyl-1,2-dihydropyridin-4-yl)-6-methyl-1-tosyl-1,6-dihydro-7H-pyrrolo[2,3-c]pyridin-7-one (300 mg, 0.549 mmol, 1.00 equiv) and (2-fluoro-3-methoxyphenyl)boronic acid (187 mg, 1.10 mmol, 2.00 equiv) in DME (10.0 mL) and H₂O (2.0 mL) was added with XPhos Pd G3 (46.5 mg, 0.055 mmol, 0.100 equiv) and K₂CO₃ (152 mg, 1.10 mmol, 2.00 equiv). The mixture was stirred at 100° C. under nitrogen atmosphere for 2 h. The reaction was quenched with water at room temperature. The resulting mixture was extracted with EtOAc (5×50 mL). The combined organic layers were washed with brine (3×50 mL), dried over anhydrous Na₂SO₄. After filtration, the filtrate was concentrated under reduced pressure. This resulted in the title compound as a black oil (500 mg, crude). The crude product was used in the next step directly without further purification. LCMS: m/z=636 [M+H]⁺

### Preparation 191: 4-(1-cyclopropyl-2-oxo-5-phenyl-1,2-dihydropyridin-4-yl)-2-(2-fluoro-3-methoxyphenyl)-6-methyl-1,6-dihydro-7H-pyrrolo[2,3-c]pyridin-7-one

To a solution of 4-(1-cyclopropyl-2-oxo-5-phenyl-1,2-dihydropyridin-4-yl)-2-(2-fluoro-3-methoxyphenyl)-6-methyl-1-tosyl-1,6-dihydro-7H-pyrrolo[2,3-c]pyridin-7-one (500 mg, 0.787 mmol, 1.00 equiv) in CH₃OH (8.0 mL) and H₂O (2.0 mL) was added NaOH (314 mg, 7.87 mmol, 10.0 equiv). The mixture was stirred at 60° C. for 1 h. The reaction was quenched with water at room temperature. The resulting mixture was extracted with CH₂Cl₂ (5×50 mL). The combined organic layers were washed with brine (3×50 mL), dried over anhydrous Na₂SO₄. After filtration, the filtrate was concentrated under reduced pressure. The crude product was purified by Prep-HPLC^{BT} to afford the title compound as a white solid (28.9 mg, 7.5%). ¹H NMR (400 MHz, DMSO-d⁶) δ 12.17 (s, 1H), 7.51 (s, 1H), 7.45-7.36 (m, 1H), 7.24-7.04 (m, 8H), 6.47 (s, 1H), 6.14 (s, 1H), 3.85 (s, 3H), 3.48 (s, 3H), 3.43-3.40 (m, 1H), 1.07-0.99 (m, 4H). LCMS^{AP}: m/z=482 [M+H]⁺

### Example 51: 2-(2,4-difluorophenyl)-4-(5-(3-(2-hydroxypropan-2-yl)phenyl)-1-methyl-2-oxo-1,2-dihydropyridin-4-yl)-6-methyl-1,6-dihydro-7H-pyrrolo[2,3-c]pyridin-7-one

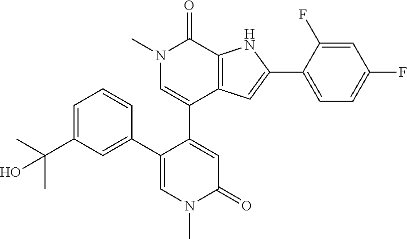 [SMILES: Cn1cc(-c2cccc(C(C)(C)O)c2)c(-c2cn(C)c(=O)c3[nH]c(-c4ccc(F)cc4F)cc23)cc1=O]

### Preparation 192: 4-bromo-5-[3-(2-hydroxypropan-2-yl) phenyl]-1-methylpyridin-2-one

To a stirred mixture of 4-bromo-5-iodo-1-methylpyridin-2-one (2.00 g, 6.30 mmol, 1.00 equiv) and 3-(2-hydroxypropan-2-yl) phenylboronic acid (1.72 g, 9.50 mmol, 1.50 equiv) in DMF (20.0 mL) and H₂O (2.00 mL) at room temperature under nitrogen atmosphere were added Na₂CO₃ (1.35 g, 12.7 mmol, 2.00 equiv) and Pd(PPh₃)₄ (736 mg, 0.600 mmol, 0.10 equiv). The resulting mixture was stirred at 100° C. under nitrogen atmosphere for 3 h. The resulting mixture was extracted with EtOAc (3×20.0 mL), dried over anhydrous Na₂SO₄. After filtration, the filtrate was concentrated under reduced pressure. The residue was purified by silica gel column chromatography, eluted with EA/MeOH (10:1) to afford the title compound as a yellow solid (800 mg, 38.9%). LCMS: m/z=324 [M+H]⁺

### Preparation 193: 4-[2-chloro-6-methyl-1-(4-methylbenzenesulfonyl)-7-oxopyrrolo[2,3-c]pyridin-4-yl]-5-[3-(2-hydroxypropan-2-yl) phenyl]-1-methylpyridin-2-one

To the mixture of 4-bromo-5-[3-(2-hydroxypropan-2-yl) phenyl]-1-methylpyridin-2-one (760 mg, 2.30 mmol, 1.00 equiv) and 2-chloro-4-(5,5-dimethyl-1,3,2-dioxaborinan-2-yl)-6-methyl-1-(4-methylbenzenesulfonyl) pyrrolo[2,3-c]pyridin-7-one (1.38 g, 3.00 mmol, 1.30 equiv) in DME (10.0 mL) and H₂O (2.00 mL) at room temperature under nitrogen atmosphere were added Na₂CO₃ (500 mg, 4.70 mmol, 2.00 equiv) and Pd(dppf)Cl₂·CH₂Cl₂ (192 mg, 0.20 mmol, 0.10 equiv). The resulting mixture was stirred at 60° C. under nitrogen atmosphere for 3 h. The resulting mixture was extracted with EtOAc (3×20.0 mL), dried over anhydrous Na₂SO₄. After filtration, the filtrate was concentrated under reduced pressure. The residue was purified by silica gel column chromatography, eluted with EA/MeOH (10:1) to afford the title compound as a yellow green oil (900 mg, 66.0%). LCMS: m/z=578 [M+H]⁺

### Preparation 194: 4-[2-(2,4-difluorophenyl)-6-methyl-1-(4-methylbenzenesulfonyl)-7-oxopyrrolo[2,3-c]pyridin-4-yl]-5-[3-(2-hydroxypropan-2-yl) phenyl]-1-methylpyridin-2-one

To a stirred mixture of 4-[2-chloro-6-methyl-1-(4-methylbenzenesulfonyl)-7-oxopyrrolo[2,3-c]pyridin-4-yl]-5-[3-(2-hydroxypropan-2-yl) phenyl]-1-methylpyridin-2-one (900 mg, 1.50 mmol, 1.00 equiv) and 2,4-difluorophenylboronic acid (368 mg, 2.30 mmol, 1.50 equiv) in DME (10.0 mL) and H₂O (2.00 mL) at room temperature under nitrogen atmosphere were added Na₂CO₃ (330 mg, 3.10 mmol, 2.00 equiv) and XPhos Pd G3 (2.93 mg, 0.004 mmol, 0.10 equiv). The resulting mixture was stirred at 100° C. under nitrogen atmosphere for 2 h. The resulting mixture was extracted with EtOAc (3×20.0 mL), dried over anhydrous Na₂SO₄. After filtration, the filtrate was concentrated under reduced pressure. This resulted in the title compound as a yellow solid (1.00 g, crude). The product was used in the next step directly without further purification. LCMS: m/z=656 [M+H]⁺

### Preparation 195: 4-[2-(2,4-difluorophenyl)-6-methyl-7-oxo-1H-pyrrolo[2, 3-c]pyridin-4-yl]-5-[3-(2-hydroxypropan-2-yl) phenyl]-1-methylpyridin-2-one

To a stirred mixture of 4-[2-(2,4-difluorophenyl)-6-methyl-1-(4-methylbenzenesulfonyl)-7-oxopyrrolo[2,3-c]pyridin-4-yl]-5-[3-(2-hydroxypropan-2-yl) phenyl]-1-methylpyridin-2-one (500 mg, 0.70 mmol, 1.00 equiv) in MeOH (4.00 mL) and H₂O (4.00 mL) was added NaOH (304 mg, 7.60 mmol, 10.0 equiv). The resulting mixture was stirred at 60° C. for 2 h. The resulting mixture was extracted with EtOAc (3×10.0 mL), dried over anhydrous Na₂SO₄. After filtration, the filtrate was concentrated under reduced pressure. The crude product (100 mg) was purified by Prep-HPLC^{BU} as a white solid (64 mg, 16.7%). 1H NMR (400 MHz, DMSO-d⁶) δ 12.20 (s, 1H), 7.91-7.90 (m, 1H), 7.84 (s, 1H), 7.31-7.28 (m, 1H), 7.23-7.06 (m, 5H), 7.04-7.02 (m, 1H), 6.49 (s, 1H), 6.09 (s, 1H), 4.81 (s, 1H), 3.56 (s, 3H), 3.48 (s, 3H), 1.12 (s, 6H). LCMS^{AV}: m/z=502 [M+H]⁺

### Example 52 (Compound B): 4-(1-(2-methoxyethyl)-2-oxo-5-phenyl-1,2-dihydropyridin-4-yl)-6-methyl-2-(1-(trifluoromethyl)-1H-pyrazol-4-yl)-1,6-dihydro-7H-pyrrolo[2,3-c]pyridin-7-one

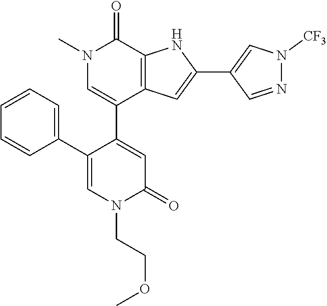 [SMILES: COCCn1cc(-c2ccccc2)c(-c2cn(C)c(=O)c3[nH]c(-c4cnn(C(F)(F)F)c4)cc23)cc1=O]

### Preparation 196: 4-bromo-5-iodo-1-(2-methoxyethyl)pyridin-2 (1H)-one

To a mixture of 4-bromo-5-iodopyridin-2 (1H)-one (5.00 g, 16.7 mmol, 1.00 equiv) and 2-bromoethyl methyl ether (2.55 g, 18.3 mmol, 1.10 equiv) in DMF (50.0 mL) was added K₂CO₃ (6.90 g, 50.1 mmol, 3.00 equiv). The resulting mixture was stirred for additional 2 h at 100° C. The resulting mixture was extracted with EtOAc (3×50 mL). The combined organic phases were dried over anhydrous Na₂SO₄. After filtration, the filtrate was concentrated under reduced pressure. The residue was purified by silica gel column chromatography, eluted with PE/EA (1:1) to afford the title compound as light-yellow solid (2.70 g, 45%). LCMS: m/z=360 [M+H]⁺.

### Preparation 197: 4-bromo-1-(2-methoxyethyl)-5-phenylpyridin-2 (1H)-one

To a stirred mixture of 4-bromo-5-iodo-1-(2-methoxyethyl)pyridin-2 (1H)-one (2.60 g, 7.28 mmol, 1.00 equiv) and phenylboronic acid (1.33 g, 10.9 mmol, 1.50 equiv) in DMF (20 mL) and H₂O (2 mL) was added Na₂CO₃ (15.5 g, 14.6 mmol, 2.00 equiv) and Pd(PPh₃)₄ (841 mg, 0.728 mmol, 0.10 equiv) at room temperature. The resulting mixture was stirred overnight at 100° C. under a nitrogen atmosphere. The resulting mixture was extracted with EtOAc (3×50 mL), dried over anhydrous Na₂SO₄. After filtration, the filtrate was concentrated under reduced pressure. The residue was purified by silica gel column chromatography, eluted with PE/EA (3:1) to afford the title compound as yellow oil (1.40 g, 63%) LCMS: m/z=310 [M+H]⁺

### Preparation 198: 2-chloro-4-(1-(2-methoxyethyl)-2-oxo-5-phenyl-1,2-dihydropyridin-4-yl)-6-methyl-1-tosyl-1,6-dihydro-7H-pyrrolo[2,3-c]pyridin-7-one

To a stirred mixture of 4-bromo-1-(2-methoxyethyl)-5-phenylpyridin-2 (1H)-one (1.40 g, 4.56 mmol, 1.00 equiv) and 2-chloro-4-(5,5-dimethyl-1,3,2-dioxaborinan-2-yl)-6-methyl-1-tosyl-1,6-dihydro-7H-pyrrolo[2,3-c]pyridin-7-one (2.66 g, 5.93 mmol, 1.30 equiv) in DME (10.0 mL) and H₂O (2.0 mL) was added Na₂CO₃ (0.97 g, 9.12 mmol, 2.00 equiv) and Pd(dppf)Cl₂·CH₂Cl₂ (0.37 g, 0.456 mmol, 0.100 equiv) at room temperature. The resulting mixture was stirred overnight at 60° C. under a nitrogen atmosphere. The resulting mixture was extracted with EtOAc (3×50 mL). The combined organic phases were dried over anhydrous Na₂SO₄. After filtration, the filtrate was concentrated under reduced pressure. The residue was purified by silica gel column chromatography, eluted with MeOH/EA (10:1) to afford the title compound as a yellow solid (1.50 g, 58%). LCMS: m/z=564 [M+H]⁺.

### Preparation 199: 4-(1-(2-methoxyethyl)-2-oxo-5-phenyl-1,2-dihydropyridin-4-yl)-6-methyl-1-tosyl-2-(1-(trifluoromethyl)-1H-pyrazol-4-yl)-1,6-dihydro-7H-pyrrolo[2,3-c]pyridin-7-one

Following the procedure described in preparation 8, 2-chloro-4-(1-(2-methoxyethyl)-2-oxo-5-phenyl-1,2-dihydropyridin-4-yl)-6-methyl-1-tosyl-1,6-dihydro-7H-pyrrolo[2,3-c]pyridin-7-one (1.40 g, 2.49 mmol, 1.00 equiv) and 4-(4,4,5,5-tetramethyl-1,3,2-dioxaborolan-2-yl)-1-(trifluoromethyl)-1H-pyrazole (0.98 g, 3.73 mmol, 1.50 equiv) was reacted to give title compound as a reddish brown oil (1.10 g, 67%). LCMS: m/z=664 [M+H]⁺.

### Preparation 200: 4-(1-(2-methoxyethyl)-2-oxo-5-phenyl-1,2-dihydropyridin-4-yl)-6-methyl-2-(1-(trifluoromethyl)-1H-pyrazol-4-yl)-1,6-dihydro-7H-pyrrolo[2, 3-c]pyridin-7-one

Following the procedure described in preparation 7, 4-(1-(2-methoxyethyl)-2-oxo-5-phenyl-1,2-dihydropyridin-4-yl)-6-methyl-1-tosyl-2-(1-(trifluoromethyl)-1H-pyrazol-4-yl)-1,6-dihydro-7H-pyrrolo[2,3-c]pyridin-7-one (1.00 g, 1.50 mmol, 1.00 equiv) was reacted to give the crude product which was purified by Prep-HPLC^{K} to afford the title compound as white solid (437.4 mg, 57.0%). LCMS^{F}: m/z=510 [M+H]⁺. ¹H NMR (400 MHz, DMSO-d⁶) δ 12.34 (s, 1H), 8.92 (s, 1H), 8.45 (s, 1H), 7.77 (s, 1H), 7.33-7.00 (m, 6H), 6.51 (s, 1H), 6.35 (s, 1H), 4.20 (t, J=5.3 Hz, 2H), 3.67 (t, J=5.3 Hz, 2H), 3.42 (s, 3H), 3.34 (s, 3H).

### Example 53: 2-(2,4-difluorophenyl)-4-(5-(3-(2-methoxyethoxy)phenyl)-1-methyl-2-oxo-1,2-dihydropyridin-4-yl)-6-methyl-1-tosyl-1,6-dihydro-7H-pyrrolo[2,3-c]pyridin-7-one

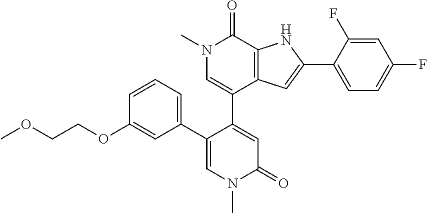 [SMILES: COCCOc1cccc(-c2cn(C)c(=O)cc2-c2cn(C)c(=O)c3[nH]c(-c4ccc(F)cc4F)cc23)c1]

### Preparation 201: 2-(2,4-difluorophenyl)-4-(5-(3-(2-methoxyethoxy)phenyl)-1-methyl-2-oxo-1,2-dihydropyridin-4-yl)-6-methyl-1-tosyl-1,6-dihydro-7H-pyrrolo[2, 3-c]pyridin-7-one

To a stirred mixture of 2-chloro-4-(5-(3-(2-methoxyethoxy)phenyl)-1-methyl-2-oxo-1,2-dihydropyridin-4-yl)-6-methyl-1-tosyl-1,6-dihydro-7H-pyrrolo[2,3-c]pyridin-7-one (500 mg, 0.842 mmol, 1.00 equiv) and 2,4-difluorophenylboronic acid (266 mg, 1.68 mmol, 2.00 equiv) in DME (25.0 mL) and H₂O (5.00 mL) under nitrogen atmosphere were added XPhos Pd G3 (71.2 mg, 0.084 mmol, 0.100 equiv) and K₂CO₃ (233 mg, 1.68 mmol, 2.00 equiv). The resulting mixture at 80° C. was stirred for 4 h. The reaction was quenched with water at room temperature. The resulting mixture was extracted with EtOAc (4×20.0 mL). The combined organic layers were washed with brine (2×10.0 mL), dried over anhydrous Na₂SO₄. After filtration, the filtrate was concentrated under reduced pressure. The residue was purified by silica gel column chromatography, eluted with EA/MeOH (5:1) to afford the title compound as a white solid (400 mg, 70.7%). LCMS: m/z=672 [M+H]⁺

### Preparation 202: 2-(2,4-difluorophenyl)-4-(5-(3-(2-methoxyethoxy)phenyl)-1-methyl-2-oxo-1,2-dihydropyridin-4-yl)-6-methyl-1,6-dihydro-7H-pyrrolo[2, 3-c]pyridin-7-one

To a mixture of 2-(2,4-difluorophenyl)-4-(5-(3-(2-methoxyethoxy)phenyl)-1-methyl-2-oxo-1,2-dihydropyridin-4-yl)-6-methyl-1-tosyl-1,6-dihydro-7H-pyrrolo[2,3-c]pyridin-7-one (380 mg, 0.566 mmol, 1.00 equiv) in MeOH (10.0 mL) and H₂O (2.0 mL) was added NaOH (226 mg, 5.66 mmol, 10.0 equiv). The resulting mixture was stirred at 60° C. for 2 h. The reaction was quenched with water at room temperature. The resulting mixture was extracted with EtOAc (4×30.0 mL). The combined organic layers were washed with brine (2×10.0 mL), dried over anhydrous Na₂SO₄. After filtration, the filtrate was concentrated under reduced pressure. The crude product was purified by Prep-HPLC^{BY} to afford the title compound as a white solid (161.1 mg, 55.0%). ¹H NMR (400 MHz, DMSO-d⁶) δ 12.26 (s, 1H), 7.96-7.90 (m, 1H), 7.89 (s, 1H), 7.35-7.30 (m, 1H) 7.29 (s, 1H), 7.24-7.16 (m, 1H), 7.07-7.03 (m, 1H), 6.80-6.79 (m, 1H), 6.74-6.71 (m, 1H), 6.67-6.64 (m, 1H), 6.47 (s, 1H), 6.17 (s, 1H), 3.91-3.89 (s, 2H), 3.54 (s, 3H), 3.50-3.47 (m, 5H), 3.22 (m, 3H). LCMS^{AR}: m/z=518 [M+H]⁺

### Example 54: 4-(5-(2,6-dimethylphenoxy)-1-methyl-2-oxo-1,2-dihydropyridin-4-yl)-2-(2-isopropylthiazol-5-yl)-6-methyl-1,6-dihydro-7H-pyrrolo[2,3-c]pyridin-7-one

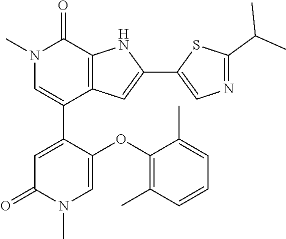 [SMILES: Cc1cccc(C)c1Oc1cn(C)c(=O)cc1-c1cn(C)c(=O)c2[nH]c(-c3cnc(C(C)C)s3)cc12]

### Preparation 203: 2-isopropyl-5-(4,4,5,5-tetramethyl-1,3,2-dioxaborolan-2-yl) thiazole

To a stirred solution of 5-bromo-2-isopropyl-1,3-thiazole (500 mg, 2.42 mmol, 1.00 equiv) in THF (25 mL) at −78° C. under nitrogen atmosphere was added n-BuLi (233 mg, 3.63 mmol, 1.50 equiv) dropwise. The resulting mixture was stirred at −78° C. under nitrogen atmosphere for 30 mins. To the above mixture at −78° C. was added 2-isopropoxy-4,4,5,5-tetramethyl-1,3,2-dioxaborolane (677 mg, 3.63 mmol, 1.50 equiv) dropwise. The resulting mixture was stirred at −78° C. under nitrogen atmosphere for 1 h. The mixture was allowed to warm up to room temperature. The reaction was quenched with water at room temperature. The reaction was quenched with water at room temperature. The resulting mixture was extracted with EtOAc (3×20 mL). The combined organic layers were washed with brine (3×9 mL), dried over anhydrous Na₂SO₄. After filtration, the filtrate was concentrated under reduced pressure. The residue was purified by Prep-TLC (PE/EA 5:1) to afford the title compound as a yellow oil (320 mg, 52.1%). LCMS: m/z=254 [M+H]⁺

### Preparation 204: 4-(5-(2,6-dimethylphenoxy)-1-methyl-2-oxo-1,2-dihydropyridin-4-yl)-2-(2-isopropylthiazol-5-yl)-6-methyl-1-tosyl-1,6-dihydro-7H-pyrrolo[2,3-c]pyridin-7-one

To a stirred solution of 2-isopropyl-5-(4,4,5,5-tetramethyl-1,3,2-dioxaborolan-2-yl)-1,3-thiazole (220 mg, 0.869 mmol, 1.67 equiv) and 4-[2-chloro-6-methyl-1-(4-methylbenzenesulfonyl)-7-oxopyrrolo[2,3-c]pyridin-4-yl]-5-(2,6-dimethylphenoxy)-1-methylpyridin-2-one (293 mg, 0.519 mmol, 1.00 equiv) in DME (3.0 mL) and H₂O (0.6 mL) at room temperature were added K₂CO₃ (143 mg, 1.04 mmol, 2.00 equiv) and XPhos Pd G3 (43 mg, 0.052 mmol, 0.10 equiv). The resulting mixture was stirred at 80° C. under nitrogen atmosphere for 1 h. The resulting mixture was extracted with EtOAc (3×30 mL). The combined organic layers were washed with brine (2×20 mL), dried over anhydrous Na₂SO₄. After filtration, the filtrate was concentrated under reduced pressure. The residue was purified by Prep-TLC (EA/MeOH 30:1) to afford the title compound as a light-yellow solid (200 mg, crude). LCMS: m/z=655 [M+H]⁺

### Preparation 205: 4-(5-(2,6-dimethylphenoxy)-1-methyl-2-oxo-1,2-dihydropyridin-4-yl)-2-(2-isopropylthiazol-5-yl)-6-methyl-1,6-dihydro-7H-pyrrolo[2,3-c]pyridin-7-one

To a stirred solution of 5-(2,6-dimethylphenoxy)-4-[2-(2-isopropyl-1,3-thiazol-5-yl)-6-methyl-1-(4-methylbenzenesulfonyl)-7-oxopyrrolo[2,3-c]pyridin-4-yl]-1-methylpyridin-2-one (200 mg, crude) in MeOH (2.0 mL) and H₂O (0.4 mL) at room temperature was added NaOH (122 mg, 3.05 mmol, 10.0 equiv). The resulting mixture was stirred at 60° C. for 6 h. The reaction was quenched with addition of water (2.0 mL). The resulting mixture was extracted with EtOAc (3×10 mL), dried with anhydrous Na₂SO₄, filtered and concentrated under reduced pressure. The residue was purified by Prep-HPLC^{BZ} to afford the title compound as a white solid (18 mg, 4.12% for two steps). ¹H NMR (400 MHz, MeOD) δ 8.07 (s, 1H), 7.58 (s, 1H), 7.12-7.02 (m, 3H), 6.80 (s, 1H), 6.69 (d, J=11.8 Hz, 2H), 3.72 (s, 3H), 3.47 (s, 3H), 3.41-3.33 (m, 1H), 2.16 (s, 6H), 1.44 (d, J=6.9 Hz, 6H). LCMS^{G}: m/z=501 [M+H]⁺

### Example 55: 4-(5-(2,6-dimethylphenoxy)-1-methyl-2-oxo-1,2-dihydropyridin-4-yl)-6-methyl-2-(2,3,5-trifluorophenyl)-1,6-dihydro-7H-pyrrolo[2,3-c]pyridin-7-one

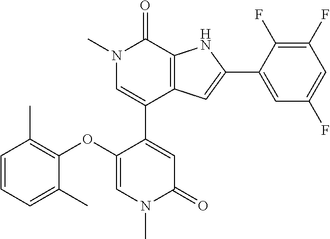 [SMILES: Cc1cccc(C)c1Oc1cn(C)c(=O)cc1-c1cn(C)c(=O)c2[nH]c(-c3cc(F)cc(F)c3F)cc12]

### Preparation 206: 4-(5-(2,6-dimethylphenoxy)-1-methyl-2-oxo-1,2-dihydropyridin-4-yl)-6-methyl-1-tosyl-2-(2,3,5-trifluorophenyl)-1,6-dihydro-7H-pyrrolo[2,3-c]pyridin-7-one

To a stirred solution of 4-[2-chloro-6-methyl-1-(4-methylbenzenesulfonyl)-7-oxopyrrolo[2,3-c]pyridin-4-yl]-5-(2,6-dimethylphenoxy)-1-methylpyridin-2-one (200 mg, 0.355 mmol, 1.00 equiv) in toluene (1.60 mL) and H₂O (0.40 ml) at room temperature under nitrogen atmosphere was added K₂CO₃ (120 mg, 0.868 mmol, 2.45 equiv), 2,3,5-trifluorophenylboronic acid (160 mg, 0.910 mmol, 2.57 equiv) and XPhos Pd G3 (40 mg, 0.047 mmol, 0.13 equiv). The resulting mixture was stirred at 100° C. for additional 2 h. The resulting mixture was concentrated under reduced pressure and diluted with water (10.0 mL). The resulting mixture was extracted with EtOAc (3×20.0 mL). The combined organic layers were dried over anhydrous Na₂SO₄. After filtration, the filtrate was concentrated under reduced pressure to afford the title compound (340 mg, crude) which was used in the next step directly without further purification. LCMS: m/z=660 [M+H]⁺

### Preparation 207: 4-(5-(2,6-dimethylphenoxy)-1-methyl-2-oxo-1,2-dihydropyridin-4-yl)-6-methyl-2-(2,3,5-trifluorophenyl)-1,6-dihydro-7H-pyrrolo[2,3-c]pyridin-7-one

To a stirred solution of 5-(2,6-dimethylphenoxy)-1-methyl-4-[6-methyl-1-(4-methylbenzenesulfonyl)-7-oxo-2-(2,3,5-trifluorophenyl)pyrrolo[2,3-c]pyridin-4-yl]pyridin-2-one (340 mg, 0.515 mmol, 1.00 equiv.) in MeOH (5.00 mL) and H₂O (1.25 mL) was added NaOH (206 mg, 5.15 mmol, 10.0 equiv). The resulting mixture was stirred at 60° C. for 1 h. The resulting mixture was extracted with EtOAc (3×10.0 mL). The combined organic layers were washed with brine (1×20.0 mL), dried over anhydrous Na₂SO₄. After filtration, the filtrate was concentrated under reduced pressure. The residue was purified by RP-HPLC^{CA} to afford the title compound as a white solid (72.6 mg, 27.8%). ¹H NMR (400 MHz, MeOD) δ 7.58 (s, 1H), 7.53-7.52 (m, 1H), 7.25-7.17 (m, 1H), 7.17-7.10 (m, 2H), 7.10-7.02 (m, 1H), 7.00 (d, J=2.6 Hz, 1H), 6.80 (s, 1H), 6.67 (s, 1H), 3.73 (s, 3H), 3.47 (s, 3H), 2.15 (s, 6H). LCMS^{AS}: m/z=506 [M+H]⁺

### Example 56: 4-(5-(2,6-dimethylphenoxy)-1-methyl-2-oxo-1,2-dihydropyridin-4-yl)-2-(2-fluoro-3-methoxyphenyl)-6-methyl-1,6-dihydro-7H-pyrrolo[2,3-c]pyridin-7-one

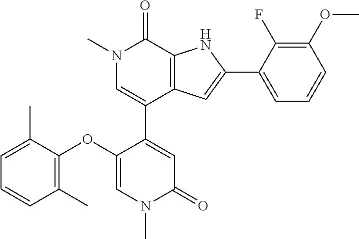 [SMILES: COc1cccc(-c2cc3c(-c4cc(=O)n(C)cc4Oc4c(C)cccc4C)cn(C)c(=O)c3[nH]2)c1F]

### Preparation 208: 4-(5-(2,6-dimethylphenoxy)-1-methyl-2-oxo-1,2-dihydropyridin-4-yl)-2-(2-fluoro-3-methoxyphenyl)-6-methyl-1-tosyl-1,6-dihydro-7H-pyrrolo[2, 3-c]pyridin-7-one

To a stirred solution of 4-[2-chloro-6-methyl-1-(4-methylbenzenesulfonyl)-7-oxopyrrolo[2,3-c]pyridin-4-yl]-5-(2,6-dimethylphenoxy)-1-methylpyridin-2-one (100 mg, 0.177 mmol. 10 equiv) and 2-fluoro-3-methoxyphenylboronic acid (61 mg, 0.354 mmol, 2.00 equiv) in DME (2.0 mL) and water (0.40 mL) at room temperature were added XPhos Pd G3 (15.0 mg, 0.018 mmol, 0.10 equiv) and K₂CO₃ (49.0 mg, 0.354 mmol, 2.00 equiv). The resulting mixture was stirred at 100° C. under nitrogen atmosphere for additional 12 h. The resulting mixture was extracted with EtOAc (3×20 mL), dried over anhydrous Na₂SO₄. After filtration, the filtrate was concentrated under reduced pressure. The crude product was used in the next step directly without further purification. LCMS: m/z=654 [M+H]⁺

### Preparation 209: 4-(5-(2,6-dimethylphenoxy)-1-methyl-2-oxo-1,2-dihydropyridin-4-yl)-2-(2-fluoro-3-methoxyphenyl)-6-methyl-1,6-dihydro-7H-pyrrolo[2, 3-c]pyridin-7-one

To a solution of 5-(2,6-dimethylphenoxy)-4-[2-(2-fluoro-3-methoxyphenyl)-6-methyl-1-(4-methylbenzenesulfonyl)-7-oxopyrrolo[2,3-c]pyridin-4-yl]-1-methylpyridin-2-one (100 mg, 0.153 mmol, 1.00 equiv) in 1,4-dioxane (2.0 mL) and H₂O (0.5 mL) was added NaOH (61 mg, 1.53 mmol, 10.0 equiv). The resulting mixture was stirred at 60° C. under nitrogen atmosphere for 12 h. The resulting mixture was extracted with EtOAc (3×15 mL), dried over anhydrous Na₂SO₄. After filtration, the filtrate was concentrated under reduced pressure. The crude product (100 mg) was purified by Prep-HPLC^{CB} to afford the title compound as a white solid (33.7 mg, 44.1%). ¹H NMR (400 MHz, DMSO-d₆) δ 12.43 (s, 1H), 7.61 (t, J=6.7 Hz, 1H), 7.52 (s, 1H), 7.24-7.12 (m, 2H), 7.11-7.09 (m, 2H), 7.04-7.02 (m, 1H), 6.81-6.76 (m, 1H), 6.66 (s, 1H), 6.53 (s, 1H), 3.88 (s, 3H), 3.61 (s, 3H), 3.30 (s, 3H), 2.09 (s, 6H). LCMS^{AT}: m/z=500 [M+H]⁺

### Example 57: 4-(5-(2,6-dimethylphenoxy)-1-methyl-2-oxo-1,2-dihydropyridin-4-yl)-2-(2-fluoro-3-(hydroxymethyl)phenyl)-6-methyl-1,6-dihydro-7H-pyrrolo[2,3-c]pyridin-7-one

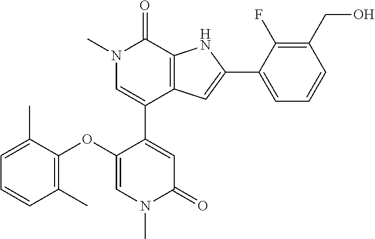 [SMILES: Cc1cccc(C)c1Oc1cn(C)c(=O)cc1-c1cn(C)c(=O)c2[nH]c(-c3cccc(CO)c3F)cc12]

### Preparation 210: 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2, 3-c]pyridin-7-one

To a stirred mixture of 2-chloro-6-methyl-1-(4-methylbenzenesulfonyl)-4-(4,4,5,5-tetramethyl-1,3,2-dioxaborolan-2-yl)pyrrolo[2,3-c]pyridin-7-one (1.50 g, 3.24 mmol, 1.00 equiv) and 4-bromo-5-(2,6-dimethylphenoxy)-1-methylpyridin-2-one (1.20 g, 3.89 mmol, 1.20 equiv) in DME (1.50 mL) and H₂O (0.3 mL) were added Na₂CO₃ (690 mg, 6.48 mmol, 2.0 equiv) and Pd(dppf)Cl₂ (260 mg, 0.324 mmol, 0.1 equiv). The resulting mixture was stirred at 60° C. under nitrogen atmosphere overnight. The mixture was allowed to cool down to room temperature and quenched with water. The resulting mixture was extracted with EtOAc (3×50 mL). The combined organic layers were washed with water and brine, dried over anhydrous Na₂SO₄. After filtration, the filtrate was concentrated under reduced pressure. The residue was purified by silica gel column chromatography, eluted with EA/MeOH (12:1) to afford the title compound as a yellow solid (910 mg, 49.8%). LCMS: m/z=564 [M+H]⁺

### Preparation 211: 4-(5-(2,6-dimethylphenoxy)-1-methyl-2-oxo-1,2-dihydropyridin-4-yl)-2-(2-fluoro-3-(hydroxymethyl) phenyl)-6-methyl-1-tosyl-1,6-dihydro-7H-pyrrolo[2,3-c]pyridin-7-on

To a stirred mixture of 4-[2-chloro-6-methyl-1-(4-methylbenzenesulfonyl)-7-oxopyrrolo[2,3-c]pyridin-4-yl]-5-(2,6-dimethylphenoxy)-1-methylpyridin-2-one (100 mg, 0.177 mmol, 1.00 equiv) and [2-fluoro-3-(4,4,5,5-tetramethyl-1,3,2-dioxaborolan-2-yl)phenyl]methanol (89.4 mg, 0.354 mmol, 2.00 equiv) in DME (0.5 mL) and H₂O (0.1 mL) at room temperature under nitrogen atmosphere were added KCO₃ (49.0 mg, 0.354 mmol, 2.00 equiv) and X Phos Pd G3 (15.0 mg, 0.018 mmol, 0.10 equiv). The resulting mixture was stirred at 60° C. under nitrogen atmosphere overnight. The resulting mixture was extracted with EtOAc (3×10 mL). The combined organic phases were dried over anhydrous Na₂SO₄. After filtration, the filtrate was concentrated under reduced pressure. The crude product was used in the next step directly without further purification. LCMS: m/z=654 [M+H]⁺

### Preparation 212: 4-(5-(2,6-dimethylphenoxy)-1-methyl-2-oxo-1,2-dihydropyridin-4-yl)-2-(2-fluoro-3-(hydroxymethyl)phenyl)-6-methyl-1,6-dihydro-7H-pyrrolo[2,3-c]pyridin-7-one

To a stirred mixture of 5-(2,6-dimethylphenoxy)-4-{2-[2-fluoro-3-(hydroxymethyl) phenyl]-6-methyl-1-(4-methylbenzenesulfonyl)-7-oxopyrrolo[2,3-c]pyridin-4-yl}-1-methylpyridin-2-one (70 mg, 0.107 mmol, 1.00 equiv) in dioxane (1.80 mL) and H₂O (0.2 mL) was added NaOH (43 mg, 1.07 mmol, 10.0 equiv). The mixture was stirred at 60° C. under nitrogen atmosphere for 3 h. The resulting mixture was extracted with EtOAc (3×10 mL), dried over anhydrous Na₂SO₄. After filtration, the filtrate was concentrated under reduced pressure. The crude product (40 mg) was purified by Prep-HPLC^{CC} to afford the title compound as a white solid (13 mg, 24.3%). ¹H NMR (400 MHz, DMSO-d⁶) δ 12.35 (s, 1H), 7.97 (t, J=7.5 Hz, 1H), 7.53 (s, 1H), 7.45 (t, J=7.1 Hz, 1H), 7.26 (t, J=7.7 Hz, 1H), 7.10-7.06 (m, 2H), 7.06-7.02 (m, 1H), 6.80 (s, 1H), 6.66 (s, 1H), 6.54 (s, 1H), 5.32 (t, J=5.7 Hz, 1H), 4.61 (d, J=5.7 Hz, 2H), 3.61 (s, 3H), 3.32 (s, 3H), 2.10 (s, 6H). LCMS^{C}: m/z=500 [M+H]⁺

### Example 58: 4-(5-(2,6-dimethylphenoxy)-1-methyl-2-oxo-1,2-dihydropyridin-4-yl)-2-(2-fluoro-4-(hydroxymethyl)phenyl)-6-methyl-1,6-dihydro-7H-pyrrolo[2,3-c]pyridin-7-one

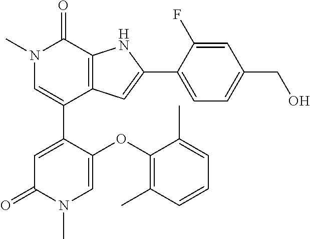 [SMILES: Cc1cccc(C)c1Oc1cn(C)c(=O)cc1-c1cn(C)c(=O)c2[nH]c(-c3ccc(CO)cc3F)cc12]

### Preparation 213: 4-(5-(2,6-dimethylphenoxy)-1-methyl-2-oxo-1,2-dihydropyridin-4-yl)-2-(2-fluoro-4-(hydroxymethyl) phenyl)-6-methyl-1-tosyl-1,6-dihydro-7H-pyrrolo[2,3-c]pyridin-7-one

To a stirred mixture of 4-[2-chloro-6-methyl-1-(4-methylbenzenesulfonyl)-7-oxopyrrolo[2,3-c]pyridin-4-yl]-5-(2,6-dimethylphenoxy)-1-methylpyridin-2-one (100 mg, 0.177 mmol, 1.00 equiv) and [3-fluoro-4-(4,4,5-trimethyl-1,3,2-dioxaborolan-2-yl)phenyl]methanol (84.4 mg, 0.354 mmol, 2.00 equiv) in DME (1.0 mL) and H₂O (0.2 mL) were added K₂CO₃ (49.0 mg, 0.354 mmol, 2.00 equiv) and XPhos Pd G3 (15.0 mg, 0.018 mmol, 0.10 equiv). The resulting mixture was stirred at 100° C. under nitrogen atmosphere overnight. The resulting mixture was extracted with EtOAc (3×10 mL), dried over anhydrous Na₂SO₄. After filtration, the filtrate was concentrated under reduced pressure. The crude product was used in the next step directly without further purification. LCMS: m/z=654 [M+H]⁺

### Preparation 214: 5-(2,6-dimethylphenoxy)-4-{2-[2-fluoro-4-(hydroxymethyl)phenyl]-6-methyl-7-oxo-1H-pyrrolo[2, 3-c]pyridin-4-yl}-1-methylpyridin-2-one

To a stirred mixture of 5-(2,6-dimethylphenoxy)-4-{2-[2-fluoro-4-(hydroxymethyl)phenyl]-6-methyl-1-(4-methylbenzenesulfonyl)-7-oxopyrrolo[2,3-c]pyridin-4-yl}-1-methylpyridin-2-one (100 mg, 0.153 mmol, 1.00 equiv) in dioxane (0.8 mL) and H₂O (0.2 mL) was added NaOH (62 mg, 1.53 mmol, 10.0 equiv). The resulting mixture was stirred at 60° cand extracted with EtOAc (3×10 mL). The combined organic phases were washed with brine dried over anhydrous Na₂SO₄, filtered and concentrated under reduced pressure. The crude product (40 mg) was purified by Prep-HPLC^{CD} to afford the title compound as a white solid (4 mg, 5.23%). ¹H NMR (400 MHz, MeOD) δ 7.83 (t, J=8.0 Hz, 1H), 7.57 (s, 1H), 7.27-7.24 (m, 2H), 7.14-7.01 (m, 3H), 6.91 (s, 1H), 6.81 (s, 1H), 6.67 (s, 1H), 4.66 (s, 2H), 3.73 (s, 3H), 3.47 (s, 3H), 2.15 (s, 6H). LCMS^{AU}: m/z=500 [M+H]⁺

### Example 59 6-methyl-4-(2-oxo-5-phenyl-1-(2-(trifluoromethoxy)ethyl)-1,2-dihydropyridin-4-yl)-2-(1-(trifluoromethyl)-1H-pyrazol-4-yl)-1,6-dihydro-7H-pyrrolo[2,3-c]pyridin-7-one

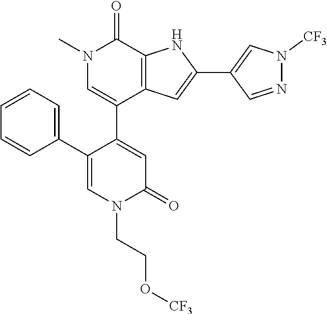 [SMILES: Cn1cc(-c2cc(=O)n(CCOC(F)(F)F)cc2-c2ccccc2)c2cc(-c3cnn(C(F)(F)F)c3)[nH]c2c1=O]

### Preparation 215: 4-bromo-1-(2-hydroxyethyl)-5-iodopyridin-2 (1H)-one

To a stirred solution of 4-bromo-5-iodo-1H-pyridin-2-one (5.00 g, 16.6 mmol, 1.00 equiv) and 2-bromoethanol (2.29 g, 18.3 mmol, 1.10 equiv) in acetone (150 mL) at room temperature was added K₂CO₃ (6.91 g, 50.0 mmol, 3.00 equiv). The resulting mixture was stirred at 60° C. overnight. The resulting mixture was filtered, the filter cake was washed with EtOAc (3×10.0 mL). The filtrate was concentrated under reduced pressure. The residue was purified by silica gel column chromatography (eluted with PE/EA (1:2)) to afford the title compound as a brown solid (3.00 g, 47.0%). LCMS: m/z=346 [M+H]⁺

### Preparation 216: 4-bromo-1-(2-hydroxyethyl)-5-phenylpyridin-2 (1H)-one

To a stirred mixture of 4-bromo-1-(2-hydroxyethyl)-5-iodopyridin-2 (1H)-one (4.00 g, 11.6 mmol, 1.00 equiv) and phenyl boronic acid (1.70 g, 14.0 mmol, 1.20 equiv) in toluene (10.0 mL), EtOH (50.0 mL) and H₂O (10.0 mL) were added Na₂CO₃ (2.47 g, 23.3 mmol, 2.00 equiv) and Pd(PPh₃)₄ (1.34 g, 1.16 mmol, 0.10 equiv). The resulting mixture was stirred at 80° C. under nitrogen atmosphere for 2 h. The resulting mixture was extracted with EtOAc (3×50.0 mL), dried over anhydrous Na₂SO₄, filtered, and concentrated under reduced pressure. The crude product was purified by Prep-HPLC^{CE} to afford the title compound as a yellow solid (2.50 g, 73.1%). LCMS: m/z=296 [M+H]⁺

### Preparation 217: 4-bromo-5-phenyl-1-(2-(trifluoromethoxy)ethyl)pyridin-2 (1H)-one and 4-bromo-1-(2-hydroxyethyl)-5-phenyl-3-(trifluoromethyl)pyridin-2 (1H)-one

To a stirred mixture of 4-bromo-1-(2-hydroxyethyl)-5-phenylpyridin-2-one (1.00 g, 3.40 mmol, 1.00 equiv) and 2-fluoropyridine (900 mg, 10.2 mmol, 3.00 equiv) in EtOAc (15.0 mL) were added TMSCF₃ (1.45 g, 10.2 mmol, 3.00 equiv), silver trifluoromethanesulfonate (2.62 g, 10.2 mmol, 3.00 equiv), 1-ChloroMethyl-4-fluoro-1,4-diazoniabicyclo[2.2.2]octane bis(tetrafluoroborate) (1.80 g, 5.10 mmol, 1.50 equiv) and KF (790 mg, 13.6 mmol, 4.00 equiv). The resulting mixture was stirred at room temperature under nitrogen atmosphere overnight. The resulting mixture was filtered, the filter cake was washed with EtOAc (3×10.0 mL). The filtrate was concentrated under reduced pressure. The residue was purified by silica gel column chromatography (eluted with PE/EA (1:1) to afford 4-bromo-5-phenyl-1-[2-(trifluoromethoxy) ethyl]pyridin-2-one as a yellow oil (230 mg, 18.6%) and 4-bromo-1-(2-hydroxyethyl)-5-phenyl-3-(trifluoromethyl) pyridin-2-one as a yellow solid (400 mg, 10.8%). LCMS: m/z=364 [M+H]⁺

### Preparation 218: 2-chloro-6-methyl-4-(2-oxo-5-phenyl-1-(2-(trifluoromethoxy)ethyl)-1,2-dihydropyridin-4-yl)-1-tosyl-1,6-dihydro-7H-pyrrolo[2,3-c]pyridin-7-one

To a stirred mixture of 4-bromo-5-phenyl-1-[2-(trifluoromethoxy)ethyl]pyridin-2-one (200 mg, 0.55 mmol, 1.00 equiv) and 2-chloro-4-(5,5-dimethyl-1,3,2-dioxaborinan-2-yl)-6-methyl-1-(4-methylbenzenesulfonyl)pyrrolo[2,3-c]pyridin-7-one (371 mg, 0.82 mmol, 1.50 equiv) in DME (5.00 mL) and H₂O (1.00 mL) were added Na₂CO₃ (117 mg, 1.10 mmol, 2.00 equiv) and Pd(dppf)Cl₂·CH₂Cl₂ (44.9 mg, 0.05 mmol, 0.10 equiv). The resulting mixture was stirred at 60° C. under nitrogen atmosphere overnight. The resulting mixture was extracted with EtOAc (3×10.0 mL), dried over anhydrous Na₂SO₄, filtered and concentrated under reduced pressure. The crude product was purified by Prep-HPCL^{BW} to afford the title compound as a brown-yellow solid (120 mg, 35.1%). LCMS: m/z=618 [M+H]⁺

### Preparation 219: 6-methyl-4-(2-oxo-5-phenyl-1-(2-(trifluoromethoxy)ethyl)-1,2-dihydropyridin-4-yl)-1-tosyl-2-(1-(trifluoromethyl)-1H-pyrazol-4-yl)-1,6-dihydro-7H-pyrrolo[2,3-c]pyridin-7-one

To a stirred mixture of 4-[2-chloro-6-methyl-1-(4-methylbenzenesulfonyl)-7-oxopyrrolo[2,3-c]pyridin-4-yl]-5-phenyl-1-[2-(trifluoromethoxy)ethyl]pyridin-2-one (100 mg, 0.16 mmol, 1.00 equiv) and 4-(4,4,5,5-tetramethyl-1,3,2-dioxaborolan-2-yl)-1-(trifluoromethyl)pyrazole (84.8 mg, 0.32 mmol, 2.00 equiv) in DME (1.0 mL) and H₂O (0.2 mL) were added K₂CO₃ (44.7 mg, 0.32 mmol, 2.00 equiv) and XPhos Pd G3 (1.37 mg, 0.02 mmol, 0.10 equiv). The resulting mixture was stirred at 60° C. under nitrogen atmosphere for 2 h. The resulting mixture was extracted with EtOAc (3×10.0 mL), dried over anhydrous Na₂SO₄, filtered and concentrated under reduced pressure. The residue was purified by Prep-TLC (MeOH/EA 1:30) to afford the title compound as a brown solid (80.0 mg, 68.8%). LCMS: m/z=718 [M+H]⁺

### Preparation 220: 6-methyl-4-(2-oxo-5-phenyl-1-(2-(trifluoromethoxy)ethyl)-1,2-dihydropyridin-4-yl)-2-(1-(trifluoromethyl)-1H-pyrazol-4-yl)-1,6-dihydro-7H-pyrrolo[2,3-c]pyridin-7-one

To a stirred solution of 4-[6-methyl-1-(4-methylbenzenesulfonyl)-7-oxo-2-[1-(trifluoromethyl) pyrazol-4-yl]pyrrolo[2,3-c]pyridin-4-yl]-5-phenyl-1-[2-(trifluoromethoxy)ethyl]pyridin-2-one (70.0 mg, 0.09 mmol, 1.00 equiv) in MeOH (0.80 mL) and H₂O (0.20 mL) was added NaOH (39.0 mg, 0.97 mmol, 10.0 equiv). The resulting mixture was stirred at 60° C. for 2 h. The resulting mixture was extracted with EtOAc (3×10.0 mL), dried over anhydrous Na₂SO₄, filtered and concentrated under reduced pressure. The crude product (50.0 mg) was purified by Prep-HPLC^{CF} to afford the title compound as a white solid (14.5 mg, 26.3%). ¹H NMR (400 MHz, CDCl₃) δ 1219 (s, 1H), 8.48 (s, 1H), 8.19 (s, 1H), 7.39 (s, 1H), 7.26-7.22 (m, 3H), 7.10 (m, 2H), 6.85 (s, 1H), 6.59 (s, 1H), 6.41 (s, 1H), 4.42-4.34 (m, 4H), 3.49 (s, 3H). LCMS^{AW}: m/z=564 [M+H]⁺

### Example 60 4-(1-(2-hydroxyethyl)-2-oxo-5-phenyl-1,2-dihydropyridin-4-yl)-6-methyl-2-(1-(trifluoromethyl)-1H-pyrazol-4-yl)-1,6-dihydro-7H-pyrrolo[2,3-c]pyridin-7-one

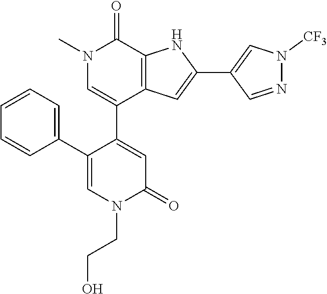 [SMILES: Cn1cc(-c2cc(=O)n(CCO)cc2-c2ccccc2)c2cc(-c3cnn(C(F)(F)F)c3)[nH]c2c1=O]

### Preparation 221: 2-chloro-4-(1-(2-hydroxyethyl)-2-oxo-5-phenyl-1,2-dihydropyridin-4-yl)-6-methyl-1-tosyl-1,6-dihydro-7H-pyrrolo[2,3-c]pyridin-7-one

To a stirred mixture of 4-bromo-1-(2-hydroxyethyl)-5-phenylpyridin-2 (1H)-one (500 mg, 1.70 mmol, 1.00 equiv) and 2-chloro-4-(5,5-dimethyl-1,3,2-dioxaborinan-2-yl)-6-methyl-1-tosyl-1,6-dihydro-7H-pyrrolo[2,3-c]pyridin-7-one (1.14 g, 2.55 mmol, 1.50 equiv) in H₂O (3.00 mL) and DME (15.0 mL) were added Na₂CO₃ (360 mg, 3.40 mmol, 2.00 equiv) and Pd(dppf)Cl₂ (124 mg, 0.170 mmol, 0.100 equiv). The resulting mixture was stirred at 60° C. under nitrogen atmosphere for 2 h. The resulting mixture was filtered, the filter cake was washed with ethyl acetate (5×10.0 mL). The filtrate was concentrated under reduced pressure. The residue was purified by Prep-TLC (eluted with EA/MeOH (15:1)) to afford the title compound as a light-yellow solid (240 mg, 25.7%). LCMS: m/z=550 [M+H]⁺

### Preparation 222: 4-(1-(2-hydroxyethyl)-2-oxo-5-phenyl-1,2-dihydropyridin-4-yl)-6-methyl-1-tosyl-2-(1-(trifluoromethyl)-1H-pyrazol-4-yl)-1,6-dihydro-7H-pyrrolo[2,3-c]pyridin-7-one

To a stirred mixture of 2-chloro-4-(1-(2-hydroxyethyl)-2-oxo-5-phenyl-1,2-dihydropyridin-4-yl)-6-methyl-1-tosyl-1,6-dihydro-7H-pyrrolo[2,3-c]pyridin-7-one (220 mg, 0.400 mmol, 1.00 equiv) and 4-(4,4,5,5-tetramethyl-1,3,2-dioxaborolan-2-yl)-1-(trifluoromethyl)-1H-pyrazole (210 mg, 0.800 mmol, 2.00 equiv) in DME (2.00 mL) and H₂O (1.00 mL) were added XPhos Pd G3 (33.9 mg, 0.040 mmol, 0.100 equiv) and K₂CO₃ (111 mg, 0.800 mmol, 2.00 equiv). The resulting mixture was stirred at 80° C. under nitrogen atmosphere for 2 h. The resulting mixture was filtered, the filter cake was washed with ethyl acetate (3×10.0 mL). The filtrate was concentrated under reduced pressure. The residue was purified by Prep-TLC (eluted with EA/MeOH (12:1)) to afford the title compound as a light-yellow solid (200 mg, 76.8%). LCMS: m/z=650 [M+H]⁺

### Preparation 223: 4-(1-(2-hydroxyethyl)-2-oxo-5-phenyl-1,2-dihydropyridin-4-yl)-6-methyl-2-(1-(trifluoromethyl)-1H-pyrazol-4-yl)-1,6-dihydro-7H-pyrrolo[2,3-c]pyridin-7-one

To a solution of 4-(1-(2-hydroxyethyl)-2-oxo-5-phenyl-1,2-dihydropyridin-4-yl)-6-methyl-1-tosyl-2-(1-(trifluoromethyl)-1H-pyrazol-4-yl)-1,6-dihydro-7H-pyrrolo[2,3-c]pyridin-7-one (190 mg, 0.292 mmol, 1.00 equiv) in MeOH (4.00 mL) and H₂O (1.00 mL) was added NaOH (117 mg, 2.92 mmol, 10.0 equiv). The resulting mixture was stirred at 60° C. for 1 h. The reaction was quenched with water at room temperature. The resulting mixture was extracted with EtOAc (4×20.0 mL). The combined organic layers were washed with brine (2×10.0 mL), dried over anhydrous Na₂SO₄ filtered and concentrated under reduced pressure. The crude product was purified by Prep-HPLC^{CH} to afford the title compound as a white solid (39.2 mg, 27.1%). ¹H NMR (400 MHz, DMSO-d₆) δ 12.29 (s, 1H), 8.91 (s, 1H), 8.44 (s, 1H), 7.75 (s, 1H), 7.23-7.10 (m, 5H), 7.06 (s, 1H), 6.51 (s, 1H), 6.34 (s, 1H), 4.96-4.94 (m, 1H), 4.09-4.07 (m, 2H), 3.74-3.70 (m, 2H), 3.42 (s, 3H). LCMS^{H}: m/z=496 [M+H]⁺

### Example 61 4-(1-(2-(dimethylamino)ethyl)-2-oxo-5-phenyl-1,2-dihydropyridin-4-yl)-6-methyl-2-(1-(trifluoromethyl)-1H-pyrazol-4-yl)-1,6-dihydro-7H-pyrrolo[2,3-c]pyridin-7-one formate

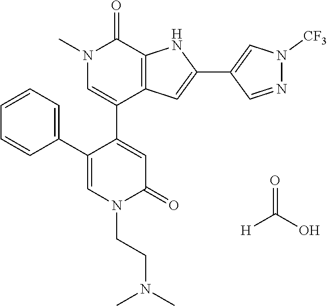 [SMILES: CN(C)CCn1cc(-c2ccccc2)c(-c2cn(C)c(=O)c3[nH]c(-c4cnn(C(F)(F)F)c4)cc23)cc1=O ; O=CO]

### Preparation 224: 4-bromo-1-(2-(dimethylamino)ethyl)-5-iodopyridin-2 (1H)-one

To a mixture of 4-bromo-5-iodopyridin-2 (1H)-one (3.00 g, 10.0 mmol, 1.00 equiv) in acetone (15.0 mL) was added Cs₂CO₃ (9.78 g, 30.0 mmol, 3.00 equiv). The mixture was stirred at room temperature for 30 min and followed by the addition of (2-bromoethyl)dimethylamine hydrobromide (4.66 g, 20.0 mmol, 2.00 equiv). The resulting mixture was stirred at 60° C. for additional 2 h. The reaction was quenched with water at room temperature. The resulting mixture was extracted with CH₂Cl₂ (5×100 mL). The combined organic layers were washed with brine (2×10 mL), dried over anhydrous Na₂SO₄, filtered and concentrated under reduced pressure. The residue was purified by Prep-HPLC_{CI} to afford the title compound as a light-yellow solid (1.84 g, 57.2%). LCMS: m/z=373 [M+H]⁺

### Preparation 225: 4-bromo-1-(2-(dimethylamino)ethyl)-5-phenylpyridin-2 (1H)-one

To a mixture of 4-bromo-1-(2-(dimethylamino)ethyl)-5-iodopyridin-2 (1H)-one (1.10 g, 2.97 mmol, 1.00 equiv) and phenyl boronic acid (0.430 g, 3.56 mmol, 1.20 equiv) in toluene (25.0 mL), EtOH (5.00 mL) and H₂O (5.00 mL) were added Pd(PPh₃)₄ (0.340 g, 0.296 mmol, 0.100 equiv) and Na₂CO₃ (0.630 g, 5.93 mmol, 2.00 equiv). The resulting mixture was stirred at 100° C. under nitrogen atmosphere for 2 h. The resulting mixture was filtered, the filter cake was washed with ethyl acetate (5×10 mL), The filtrate was concentrated under reduced pressure. The residue was purified by Prep-HPLC^{CJ} to afford the title compound as a yellow solid (490 mg, 51.45%). LCMS: m/z=323 [M+H]⁺

### Preparation 226: 2-chloro-4-(1-(2-(dimethylamino)ethyl)-2-oxo-5-phenyl-1,2-dihydropyridin-4-yl)-6-methyl-1-tosyl-1,6-dihydro-7H-pyrrolo[2,3-c]pyridin-7-one

To a stirred mixture of 4-bromo-1-(2-(dimethylamino)ethyl)-5-phenylpyridin-2 (1H)-one (480 mg, 1.49 mmol, 1.00 equiv) and 2-chloro-4-(5,5-dimethyl-1,3,2-dioxaborinan-2-yl)-6-methyl-1-(4-methylbenzenesulfonyl)pyrrolo[2,3-c]pyridin-7-one (1.00 g, 2.24 mmol, 1.50 equiv) in DME (5.00 mL) and H₂O (1.00 mL) were added Pd(dppf)Cl₂ (109 mg, 0.149 mmol, 0.100 equiv) and Na₂CO₃ (317 mg, 2.99 mmol, 2.00 equiv). The resulting mixture was stirred at 60° C. under nitrogen atmosphere for 2 h. The resulting mixture was filtered, the filter cake was washed with ethyl acetate (5×10 mL). The filtrate was concentrated under reduced pressure. The residue was purified by Prep-TLC (eluted with EA/MeOH (8:1)) to afford the title compounds as a light-yellow solid (500 mg, 57.9%). LCMS: m/z=577 [M+H]⁺

### Preparation 227: 4-(1-(2-(dimethylamino)ethyl)-2-oxo-5-phenyl-1,2-dihydropyridin-4-yl)-6-methyl-1-tosyl-2-(1-(trifluoromethyl)-1H-pyrazol-4-yl)-1,6-dihydro-7H-pyrrolo[2,3-c]pyridin-7-one

To the mixture of 2-chloro-4-(1-(2-(dimethylamino)ethyl)-2-oxo-5-phenyl-1,2-dihydropyridin-4-yl)-6-methyl-1-tosyl-1,6-dihydro-7H-pyrrolo[2,3-c]pyridin-7-one (500 mg, 0.866 mmol, 1.00 equiv) and 4-(4,4,5,5-tetramethyl-1,3,2-dioxaborolan-2-yl)-1-(trifluoromethyl)-1H-pyrazole (454 mg, 1.73 mmol, 2.00 equiv) in DME (5.00 mL) and H₂O (1.00 mL) were added XPhos Pd G3 (73.3 mg, 0.087 mmol, 0.100 equiv) and K₂CO₃ (239 mg, 1.73 mmol, 2.00 equiv). The resulting mixture was stirred at 80° C. under nitrogen atmosphere for 2 h. The resulting mixture was filtered, the filter cake was washed with ethyl acetate (3×10 mL). The filtrate was concentrated under reduced pressure. The residue was purified by Pre-TLC (eluted with EA/MeOH (8:1)) to afford the title compound as a light-yellow solid (500 mg, 85.3%). LCMS (ESI, m/z): 677 [M+H]⁺

### Preparation 228: 4-(1-(2-(dimethylamino)ethyl)-2-oxo-5-phenyl-1,2-dihydropyridin-4-yl)-6-methyl-2-(1-(trifluoromethyl)-1H-pyrazol-4-yl)-1,6-dihydro-7H-pyrrolo[2,3-c]pyridin-7-one formate

To a solution of 4-(1-(2-(dimethylamino)ethyl)-2-oxo-5-phenyl-1,2-dihydropyridin-4-yl)-6-methyl-1-tosyl-2-(1-(trifluoromethyl)-1H-pyrazol-4-yl)-1,6-dihydro-7H-pyrrolo[2,3-c]pyridin-7-one (470 mg, 0.695 mmol, 1.00 equiv) in MeOH (8.00 mL) and H₂O (2.00 mL) was added NaOH (278 mg, 6.95 mmol, 10.0 equiv). The resulting mixture was stirred at 60° C. for 1 h. The reaction was quenched with water and extracted with EtOAc (4×20 mL). The combined organic layers were washed with brine (2×10 mL), dried over anhydrous Na₂SO₄ filtered and concentrated under reduced pressure. The crude product was purified by Prep-HPLC^{CK} to afford the title compound as a white solid (119.7 mg, 32.9%). 1H NMR (400 MHz, DMSO-d₆) δ 12.32 (s, 1H), 8.91 (s, 1H), 8.44 (s, 1H), 8.16 (s, 1H), 7.82 (s, 1H), 7.24-7.16 (m, 4H), 7.13-7.10 (m, 1H), 7.08 (s, 1H), 6.49 (s, 1H), 6.34 (s, 1H), 4.12-4.09 (m, 2H), 3.42 (s, 3H), 2.64-2.61 (m, 2H), 2.28 (s, 6H). LCMS^{H}: m/z=523 [M+H]⁺.

### Example 62 6-methyl-4-(1-(2-morpholinoethyl)-2-oxo-5-phenyl-1,2-dihydropyridin-4-yl)-2-(1-(trifluoromethyl)-1H-pyrazol-4-yl)-1,6-dihydro-7H-pyrrolo[2,3-c]pyridin-7-one

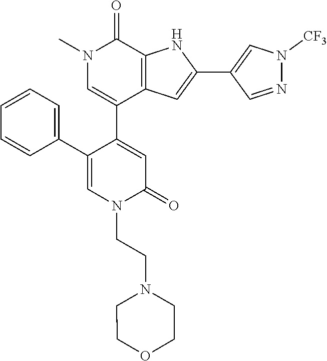 [SMILES: Cn1cc(-c2cc(=O)n(CCN3CCOCC3)cc2-c2ccccc2)c2cc(-c3cnn(C(F)(F)F)c3)[nH]c2c1=O]

### Preparation 229: 4-bromo-5-iodo-1-(2-morpholinoethyl)pyridin-2 (1H)-one

To a mixture of 4-bromo-5-iodopyridin-2 (1H)-one (3.00 g, 10.0 mmol, 1.00 equiv), 4-(2-bromoethyl) morpholine hydrobromide (5.50 g, 20.0 mmol, 2.00 equiv) in acetone (15 mL) was added K₂CO₃ (4.15 g, 30.0 mmol, 3.0 equiv). The reaction mixture was stirred at 60° C. for 2 h. The reaction was quenched with water at room temperature. The resulting mixture was extracted with CH₂Cl₂ (5×100 mL). The combined organic layers were washed with brine (2×10 mL), dried over anhydrous Na₂SO₄, filtered and concentrated under reduced pressure. The residue was purified by Prep-HPLC^{CL} to afford the title compound as a white solid (1.88 g, 45.5%). LCMS: m/z=415 [M+H]⁺

### Preparation 230: 4-bromo-1-(2-morpholinoethyl)-5-phenylpyridin-2 (1H)-one

To a stirred mixture of 4-bromo-5-iodo-1-(2-morpholinoethyl)pyridin-2 (1H)-one (1.80 g, 4.36 mmol, 1.00 equiv) and phenyl boronic acid (800 mg, 6.54 mmol, 1.50 equiv) in toluene (30.0 mL), EtOH (6.0 mL) and H₂O (6.0 mL) were added Pd(PPh₃)₄ (0.500 g, 0.436 mmol, 0.100 equiv) and Na₂CO₃ (0.920 g, 8.72 mmol, 2.00 equiv). The resulting mixture was stirred at 80° C. under nitrogen atmosphere for 2 h. The resulting mixture was filtered, the filter cake was washed with MeOH (4×10 mL). The filtrate was concentrated under reduced pressure. The residue was purified by Prep-HPLC^{C} to afford the title compound as a light-yellow solid (700 mg, 44.2%). LCMS: m/z=365 [M+H]⁺

### Preparation 231: 2-chloro-6-methyl-4-(1-(2-morpholinoethyl)-2-oxo-5-phenyl-1,2-dihydropyridin-4-yl)-1-tosyl-1,6-dihydro-7H-pyrrolo[2,3-c]pyridin-7-one

To a stirred mixture of 4-bromo-1-(2-morpholinoethyl)-5-phenylpyridin-2 (1H)-one (500 mg, 1.38 mmol, 1.00 equiv) and 2-chloro-4-(5,5-dimethyl-1,3,2-dioxaborinan-2-yl)-6-methyl-1-tosyl-1,6-dihydro-7H-pyrrolo[2,3-c]pyridin-7-one (926 mg, 2.07 mmol, 1.50 equiv) in H₂O (3.0 mL) and DME (15.0 mL) were added Pd(dppf)Cl₂ (101 mg, 0.138 mmol, 0.100 equiv) and Na₂CO₃ (292 mg, 2.75 mmol, 2.00 equiv). The resulting mixture was stirred at 60° C. under nitrogen atmosphere for 4 h. The resulting mixture was filtered, the filter cake was washed with ethyl acetate (4×10 mL). The filtrate was concentrated under reduced pressure. The residue was purified by Prep-TLC (eluted with EA/MeOH (10:1)) to afford the title compound as a yellow solid (370 mg, 43.4%). LCMS: m/z=619 [M+H]⁺

### Preparation 232: 6-methyl-4-(1-(2-morpholinoethyl)-2-oxo-5-phenyl-1,2-dihydropyridin-4-yl)-1-tosyl-2-(1-(trifluoromethyl)-1H-pyrazol-4-yl)-1,6-dihydro-7H-pyrrolo[2, 3-c]pyridin-7-one

To a stirred mixture of 2-chloro-6-methyl-4-(1-(2-morpholinoethyl)-2-oxo-5-phenyl-1,2-dihydropyridin-4-yl)-1-tosyl-1,6-dihydro-7H-pyrrolo[2,3-c]pyridin-7-one (350 mg, 0.565 mmol, 1.00 equiv) and 4-(4,4,5,5-tetramethyl-1,3,2-dioxaborolan-2-yl)-1-(trifluoromethyl)-1H-pyrazole (296 mg, 1.13 mmol, 2.00 equiv) in H₂O (1.0 mL) and DME (5.0 mL) were added XPhos Pd G3 (47.9 mg, 0.056 mmol, 0.100 equiv) and K₂CO₃ (156 mg, 1.13 mmol, 2.00 equiv). The mixture was stirred at 80° C. under nitrogen atmosphere for 3 h. The resulting mixture was filtered, the filter cake was washed with MeOH (4×10 mL). The filtrate was concentrated under reduced pressure. The residue was purified by Prep-TLC (eluted with EA/MeOH (8:1)) to afford the title compound as a light-yellow solid (310 mg, 76.3%). LCMS: m/z=719 [M+H]⁺

### Preparation 233: 6-methyl-4-(1-(2-morpholinoethyl)-2-oxo-5-phenyl-1,2-dihydropyridin-4-yl)-2-(1-(trifluoromethyl)-1H-pyrazol-4-yl)-1,6-dihydro-7H-pyrrolo[2,3-c]pyridin-7-one

To a mixture of 6-methyl-4-(1-(2-morpholinoethyl)-2-oxo-5-phenyl-1,2-dihydropyridin-4-yl)-1-tosyl-2-(1-(trifluoromethyl)-1H-pyrazol-4-yl)-1,6-dihydro-7H-pyrrolo[2,3-c]pyridin-7-one (300 mg, 0.417 mmol, 1.00 equiv) in MeOH (4 mL) and H₂O (1 mL) was added NaOH (167 mg, 4.18 mmol, 10.0 equiv). The mixture was stirred at 60° C. for 1 h. The reaction was quenched with water at room temperature. The resulting mixture was extracted with EtOAc (4×20 mL). The combined organic layers were washed with brine (2×10 mL), dried over anhydrous Na₂SO₄. The crude product (310 mg) was purified by Prep-HPLC^{CM} to afford the title compound as a white solid (83 mg, 35.2%). ¹H NMR (400 MHz, CDCl₃) δ 12.50 (s, 1H), 8.55 (s, 1H), 8.22 (s, 1H), 7.45 (s, 1H), 7.25-7.17 (m, 3H), 7.13-7.09 (m, 2H), 7.09 (s, 1H), 6.83 (s, 1H), 6.41 (s, 1H), 4.22-4.19 (m, 2H), 3.75-3.53 (m, 4H), 3.37 (s, 3H), 2.84 (s, 2H), 2.62 (s, 4H). LCMS^{BA}: m/z=565 [M+H]⁺

### Example 63 6-methyl-4-(1-(1-methylpiperidin-4-yl)-2-oxo-5-phenyl-1,2-dihydropyridin-4-yl)-2-(1-(trifluoromethyl)-1H-pyrazol-4-yl)-1,6-dihydro-7H-pyrrolo[2,3-c]pyridin-7-one

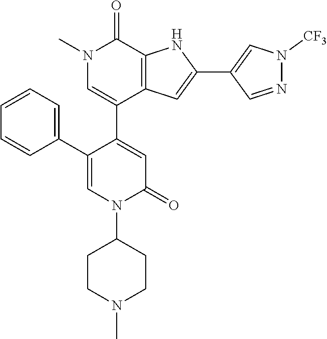 [SMILES: CN1CCC(n2cc(-c3ccccc3)c(-c3cn(C)c(=O)c4[nH]c(-c5cnn(C(F)(F)F)c5)cc34)cc2=O)CC1]

### Preparation 234: tert-butyl 4-(4-bromo-5-iodo-2-oxopyridin-1 (2H)-yl)piperidine-1-carboxylate

To a solution of 4-bromo-5-iodopyridin-2 (1H)-one (10.0 g, 33.3 mmol, 1.00 equiv) in DMF (150 mL) was added K₂CO₃ (13.8 g, 100 mmol, 3.00 equiv). The mixture was stirred at room temperature for 0.5 h and followed by the addition of tert-butyl 4-bromopiperidine-1-carboxylate (35.2 g, 133 mmol, 4.00 equiv). The mixture was stirred at 60° C. overnight. The reaction was quenched with water at room temperature. The resulting mixture was extracted with EtOAc (3×500 mL), dried over anhydrous Na₂SO₄, filtered and concentrated under reduced pressure. The residue was purified by Prep-HPLC^{CJ} to afford the title compound as a yellow oil (3.00 g crude). LCMS: m/z=485 [M+H]⁺

### Preparation 235: 4-bromo-5-iodo-1-(piperidin-4-yl) pyridin-2 (1H)-one hydrochloride

To a solution of tert-butyl 4-(4-bromo-5-iodo-2-oxopyridin-1 (2H)-yl)piperidine-1-carboxylate (3.00 g, crude) in dioxane (30.0 mL) at 0° C. was added HCl in dioxane (4 M, 30.0 mL) dropwise. The resulting mixture was stirred at room temperature for 3 h.

The reaction was quenched with water at room temperature. The resulting mixture was extracted with EtOAc (3×300 mL), dried over anhydrous Na₂SO₄, filtered and concentrated under reduced pressure. The resulting mixture was concentrated under reduced pressure to afford the title compound as a yellow solid (3.00 g, crude). LCMS (ESI, m/z): 385 [M+H]⁺ Preparation 236: 4-bromo-5-iodo-1-(1-methylpiperidin-4-yl) pyridin-2 (1H)-one

To a stirred mixture of 4-bromo-5-iodo-1-(piperidin-4-yl) pyridin-2 (1H)-one hydrochloride (3.00 g, crude) and paraformaldehyde (0.850 g, 9.40 mmol, 1.20 equiv) in DCM (40.0 mL) were added acetic acid (1.65 g, 27.4 mmol, 3.50 equiv). The resulting mixture was stirred at 80° C. for 2 h and followed by the addition of sodium cyanoboranuide (990 mg, 15.7 mmol, 2.00 equiv). The mixture was stirred at room temperature overnight. The resulting mixture was filtered, the filter cake was washed with CH₂Cl₂ (4×10.0 mL). The filtrate was concentrated under reduced pressure. The residue was purified by Prep-HPLC^{CF} to afford the title compound (350 mg, 11.3%). LCMS m/z=399 [M+H]⁺

### Preparation 237: 4-bromo-1-(1-methylpiperidin-4-yl)-5-phenylpyridin-2 (1H)-one

To a stirred mixture of 4-bromo-5-iodo-1-(1-methylpiperidin-4-yl) pyridin-2 (1H)-one (240 mg, 0.604 mmol, 1.00 equiv) and phenyl boronic acid (111 mg, 0.906 mmol, 1.50 equiv) in DME (6.0 mL) and H₂O (1.2 mL) were added Pd(PPh₃)₄ (69.9 mg, 0.060 mmol, 0.100 equiv) and Na₂CO₃ (128 mg, 1.21 mmol, 2.00 equiv). The resulting mixture was stirred at 80° C. under nitrogen atmosphere for 1.5 h. The resulting mixture was filtered. The filter cake was washed with ethyl acetate (4×10.0 mL). The filtrate was concentrated under reduced pressure. The residue was purified by Prep-HPLC^{CL} to afford the title compound as a light-yellow solid (70.0 mg, 33.4%). LCMS: m/z=349 [M+H]⁺

### Preparation 238: 2-chloro-6-methyl-4-(1-(1-methylpiperidin-4-yl)-2-oxo-5-phenyl-1,2-dihydropyridin-4-yl)-1-tosyl-1,6-dihydro-7H-pyrrolo[2,3-c]pyridin-7-one

To a stirred mixture of 4-bromo-1-(1-methylpiperidin-4-yl)-5-phenylpyridin-2 (1H)-one (70.0 mg, 0.202 mmol, 1.00 equiv) and 2-chloro-4-(5,5-dimethyl-1,3,2-dioxaborinan-2-yl)-6-methyl-1-tosyl-1,6-dihydro-7H-pyrrolo[2,3-c]pyridin-7-one (136 mg, 0.303 mmol, 1.50 equiv) in DME (2.0 mL) and H₂O (0.40 mL) were added Na₂CO₃ (42.7 mg, 0.404 mmol, 2.00 equiv) and Pd(dppf)Cl₂ (14.8 mg, 0.020 mmol, 0.100 equiv). The resulting mixture was stirred at 60° C. under nitrogen atmosphere for 2 h. The resulting mixture was filtered, the filter cake was washed with ethyl acetate (4×10.0 mL). The filtrate was concentrated under reduced pressure. The residue was purified by Prep-TLC (eluted with MeOH) to afford the title compound as a light-yellow solid (70.0 mg, 57.6%). LCMS: m/z=603 [M+H]⁺

### Preparation 239: 6-methyl-4-(1-(1-methylpiperidin-4-yl)-2-oxo-5-phenyl-1,2-dihydropyridin-4-yl)-1-tosyl-2-(1-(trifluoromethyl)-1H-pyrazol-4-yl)-1,6-dihydro-7H-pyrrolo[2,3-c]pyridin-7-one

To a stirred mixture of 2-chloro-6-methyl-4-(1-(1-methylpiperidin-4-yl)-2-oxo-5-phenyl-1,2-dihydropyridin-4-yl)-1-tosyl-1,6-dihydro-7H-pyrrolo[2,3-c]pyridin-7-one (70.0 mg, 0.116 mmol, 1.00 equiv) and 4-(4,4,5,5-tetramethyl-1,3,2-dioxaborolan-2-yl)-1-(trifluoromethyl)-1H-pyrazole (60.8 mg, 0.232 mmol, 2.00 equiv) in DME (1.0 mL) and H₂O (0.20 mL) were added XPhos Pd G3 (9.82 mg, 0.012 mmol, 0.100 equiv) and Na₂CO₃ (24.6 mg, 0.232 mmol, 2.00 equiv). The resulting mixture was stirred at 80° C. under nitrogen atmosphere for 2 h. The resulting mixture was filtered. The filter cake was washed with ethyl acetate (2×5.0 mL). The filtrate was concentrated under reduced pressure. The residue was purified by Prep-TLC (eluted with MeOH) to afford the title compound as a light-yellow solid (68.0 mg, 83.4%). LCMS: m/z=703 [M+H]⁺

### Preparation 240: 6-methyl-4-(1-(1-methylpiperidin-4-yl)-2-oxo-5-phenyl-1,2-dihydropyridin-4-yl)-2-(1-(trifluoromethyl)-1H-pyrazol-4-yl)-1,6-dihydro-7H-pyrrolo[2,3-c]pyridin-7-one

To a solution of 6-methyl-4-(1-(1-methylpiperidin-4-yl)-2-oxo-5-phenyl-1,2-dihydropyridin-4-yl)-1-tosyl-2-(1-(trifluoromethyl)-1H-pyrazol-4-yl)-1,6-dihydro-7H-pyrrolo[2,3-c]pyridin-7-one (60.0 mg, 0.085 mmol, 1.00 equiv) in MeOH (1.2 mL) and H₂O (0.30 mL) was added NaOH (34.2 mg, 0.850 mmol, 10.0 equiv). The resulting mixture was stirred at 60° C. for 1 h. The resulting mixture was filtered, the filter cake was washed with ethyl acetate (2×5.0 mL). The filtrate was concentrated under reduced pressure. The crude product was purified by Prep-HPLC^{CN} to afford the title compound as a white solid (11.4 mg, 24.3%). ¹H NMR (400 MHz, MeOD) δ 8.52 (s, 1H), 8.14 (s, 1H), 7.73 (s, 1H), 7.21-7.17 (m, 5H), 6.90 (s, 1H), 6.74 (s, 1H), 6.33 (s, 1H), 4.87-4.86 (m, 1H), 3.52 (s, 3H), 3.10-3.07 (m, 2H), 2.37 (s, 3H), 2.30-2.27 (m, 2H), 2.07-2.00 (m, 4H). LCMS^{BA} m/z=549 [M+H]⁺

### Example 64 4-(1-(3-hydroxycyclobutyl)-2-oxo-5-phenyl-1,2-dihydropyridin-4-yl)-6-methyl-2-(1-(trifluoromethyl)-1H-pyrazol-4-yl)-1,6-dihydro-7H-pyrrolo[2,3-c]pyridin-7-one

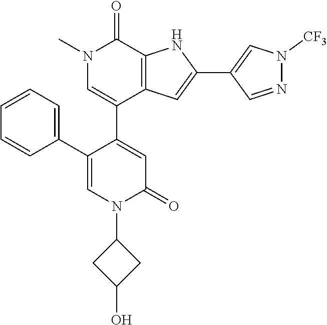 [SMILES: Cn1cc(-c2cc(=O)n(C3CC(O)C3)cc2-c2ccccc2)c2cc(-c3cnn(C(F)(F)F)c3)[nH]c2c1=O]

### Preparation 241: 4-bromo-5-iodo-1-(3-oxocyclobutyl) pyridin-2-one

To a stirred solution of 4-bromo-5-iodo-1H-pyridin-2-one (5.00 g, 16.6 mmol, 1.00 equiv) and 3-bromocyclobutan-1-one (3.73 g, 25.0 mmol, 1.50 equiv) in THF (80 mL) at room temperature was added K₂CO₃ (3.46 g, 25.0 mmol, 1.50 equiv). The resulting mixture was stirred at room temperature overnight. The resulting mixture was extracted with EtOAc (3×50 mL). The combined organic layers were washed with brine (3×30 mL), dried over anhydrous Na₂SO₄, filtered and concentrated under reduced pressure. The residue was purified by silica gel column chromatography (eluted with PE/EA (1:1)) to afford the title compound as a brown oil (2.90 g, 41.1%). LCMS: m/z=370 [M+H]⁺

### Preparation 242: 4-bromo-1-(3-hydroxycyclobutyl)-5-iodopyridin-2-one

To a stirred solution of 4-bromo-5-iodo-1-(3-oxocyclobutyl) pyridin-2-one (2.80 g, 7.60 mmol, 1.00 equiv) in MeOH (28 mL) at 0° C. was added NaBH₄ (720 mg, 19.0 mmol, 2.50 equiv). The resulting mixture was stirred at room temperature for 1 h. The reaction was quenched with water at room temperature. The resulting mixture was extracted with EtOAc (3×30 mL). The combined organic layers were washed with brine (3×6 mL), dried over anhydrous Na₂SO₄, filtered and concentrated under reduced pressure. The residue was purified by silica gel column chromatography (eluted with PE/EA (1:9)) to afford the title compound as a brown solid (2.36 g, 84.0%). LCMS: m/z=372 [M+H]⁺

### Preparation 243: 4-bromo-1-(3-hydroxycyclobutyl)-5-phenylpyridin-2-one

To a stirred solution of 4-bromo-1-(3-hydroxycyclobutyl)-5-iodopyridine-2-one (1.30 g, 3.51 mmol, 1.00 equiv) and phenyl boronic acid (514 mg, 4.21 mmol, 1.20 equiv) in toluene (6.0 mL), H₂O 1.0 mL) and EtOH (1.0 mL) were added Pd(PPH₃)₄ (406 mg, 0.351 mmol, 0.10 equiv) and Na₂CO₃ (744 mg, 7.02 mmol, 2.00 equiv). The resulting mixture was stirred at 80° C. under nitrogen atmosphere for 5 hr. The resulting mixture was extracted with EtOAc (3×10 mL). The combined organic layers were washed with brine (3×6 mL), dried over anhydrous Na₂SO₄ filtered and concentrated under reduced pressure. The crude product was purified by Prep-HPLC^{T} to afford the title compound as a brown solid (40 mg, 35.5%). LCMS: m/z=322 [H+H]⁺

### Preparation 244: 4-[2-chloro-6-methyl-1-(4-methylbenzenesulfonyl)-7-oxopyrrolo[2,3-c]pyridin-4-yl]-1-(3-hydroxycyclobutyl)-5-phenylpyridin-2-one

To a stirred solution of 4-bromo-1-(3-hydroxycyclobutyl)-5-phenylpyridin-2-one (700 mg, 2.18 mmol, 1.00 equiv) and 2-chloro-4-(5,5-dimethyl-1,3,2-dioxaborinan-2-yl)-6-methyl-1-(4-methylbenzenesulfonyl) pyrrolo[2,3-c]pyridin-7-one (1.47 g, 3.27 mmol, 1.50 equiv) in DME (10.0 mL) and H₂O (2.0 mL) were added Na₂CO₃ (463 mg, 4.37 mmol, 2.00 equiv) and Pd(dppf)Cl₂·CH₂Cl₂ (178 mg, 0.219 mmol, 0.10 equiv). The resulting mixture was stirred at 60° C. under nitrogen atmosphere overnight. The resulting mixture was extracted with EtOAc (3×10 mL). The combined organic layers were washed with brine (3×10 mL), dried over anhydrous Na₂SO₄ filtered and concentrated under reduced pressure. The residue was purified by silica gel column chromatography (eluted with MeOH/EA (1:10)) to afford the title compound as a brown solid (380 mg, crude). LCMS m/z=576 [M+H]⁺

### Preparation 245: 1-(3-hydroxycyclobutyl)-4-[6-methyl-1-(4-methylbenzenesulfonyl)-7-oxo-2-[1-(trifluoromethyl) pyrazol-4-yl]pyrrolo[2,3-c]pyridin-4-yl]-5-phenylpyridin-2-one

To a stirred solution of 4-[2-chloro-6-methyl-1-(4-methylbenzenesulfonyl)-7-oxopyrrolo[2,3-c]pyridin-4-yl]-1-(3-hydroxycyclobutyl)-5-phenylpyridin-2-one (360 mg, 0.625 mmol, 1.00 equiv) and 4-(4,4,5,5-tetramethyl-1,3,2-dioxaborolan-2-yl)-1-(trifluoromethyl)pyrazole (245 mg, 0.938 mmol, 1.50 equiv) in DME (6.0 mL) and H₂O (1.0 mL) were added XPhos Pd G3 (53 mg, 0.063 mmol, 0.100 equiv) and K₂CO₃ (172 mg, 1.25 mmol, 2.00 equiv). The resulting mixture was stirred at 80° C. under nitrogen atmosphere overnight. The resulting mixture was extracted with EtOAc (3×5 mL). The combined organic layers were washed with brine (3×20 mL), dried over anhydrous Na₂SO₄, filtered and concentrated under reduced pressure. The residue was purified by silica gel column chromatography (eluted with MeOH/EA (1:10)) to afford the title compound as a brown solid (120 mg, crude). LCMS m/z=676 [M+H]⁺

### Preparation 246: 1-(3-hydroxycyclobutyl)-4-{6-methyl-7-oxo-2-[1-(trifluoromethyl) pyrazo l-4-yl]-1H-pyrrolo[2,3-c]pyridin-4-yl}-5-phenylpyridin-2-one

To a solution of 1-(3-hydroxycyclobutyl)-4-[6-methyl-1-(4-methylbenzenesulfonyl)-7-oxo-2-[1-(trifluoromethyl)pyrazol-4-yl]pyrrolo[2,3-c]pyridin-4-yl]-5-phenylpyridin-2-one (100 mg, 0.148 mmol, 1.00 equiv) in MeOH (3.0 mL) and H₂O (0.6 mL) were added NaOH (59 mg, 1.48 mmol, 10.00 equiv). The resulting mixture was stirred at 60° C. under nitrogen atmosphere for 2 h. The resulting mixture was extracted with EtOAc (3×2 mL). The combined organic layers were washed with brine (3×20 mL), dried over anhydrous Na₂SO₄, filtered and concentrated under reduced pressure. The crude product was purified by Prep-HPLC^{CO} to afford the title compound as a white solid (31.4 mg, 36.2%). ¹H NMR (400 MHz, DMSO-d₆) δ 12.30 (s, 1H), 8.90 (s, 1H), 8.43 (s, 1H), 7.78 (s, 1H), 7.27-7.08 (m, 5H), 7.05 (s, 1H), 6.48 (s, 1H), 6.36 (s, 1H), 5.23 (br, 1H), 4.83-4.70 (m, 1H), 4.05-3.93 (m, 1H), 3.42 (s, 3H), 2.85-2.71 (m, 2H), 2.25-2.13 (m, 2H). LCMSBB: m/z=522 [M+H]⁺.

### Example 65 4-[3-fluoro-2-(1-isopropylpyrazol-4-yl)-6-methyl-7-oxo-1H-pyrrolo[2,3-c]pyridin-4-yl]-1-methyl-5-phenylpyridin-2-one

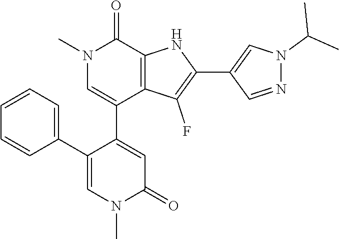 [SMILES: CC(C)n1cc(-c2[nH]c3c(=O)n(C)cc(-c4cc(=O)n(C)cc4-c4ccccc4)c3c2F)cn1]

### Preparation 247:4-[3-fluoro-2-(1-isopropylpyrazol-4-yl)-6-methyl-7-oxo-1H-pyrrolo[2, 3-c]pyridin-4-yl]-1-methyl-5-phenylpyridin-2-one

To a mixture of 4-[2-(1-isopropylpyrazol-4-yl)-6-methyl-7-oxo-1H-pyrrolo[2,3-c]pyridin-4-yl]-1-methyl-5-phenylpyridin-2-one (80 mg, 0.182 mmol, 1.00 equiv) in DCM (1.0 mL) was added 1-fluoro-2,6-dichloropyridinium tetrafluoroborate (69.3 mg, 0.273 mmol, 1.50 equiv). The resulting mixture was stirred at room temperature for 1 h. The resulting mixture was extracted with EtOAc (3×10 mL), dried over anhydrous Na₂SO₄, filtered and concentrated under reduced pressure. The crude product (50 mg) was purified by Prep-HPLC^{CP} to afford the title compound as a yellow solid (5.2 mg, 6.24%).

¹H NMR (400 MHz, CDCl₃) δ 10.70 (br, 1H), 7.94 (s, 1H), 7.81 (s, 1H), 7.35 (s, 1H), 7.17-7.16 (m, 3H), 7.15-7.09 (m, 2H), 6.76 (s, 1H), 6.63 (s, 1H), 4.57-4.51 (m, 1H), 3.67 (s, 3H), 3.55 (s, 3H), 1.55-1.52 (m, 6H). LCMS^{B}C: m/z=458 [M+H]⁺

### Example 66 6-methyl-4-(1-methyl-6-oxo-1,6-dihydro-[3,3′-bipyridin]-4-yl)-1-tosyl-2-(1-(trifluoromethyl)-1H-pyrazol-4-yl)-1,6-dihydro-7H-pyrrolo[2,3-c]pyridin-7-one

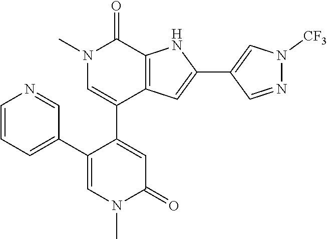 [SMILES: Cn1cc(-c2cccnc2)c(-c2cn(C)c(=O)c3[nH]c(-c4cnn(C(F)(F)F)c4)cc23)cc1=O]

### Preparation 248: 4-bromo-1-methyl-[3,3′-bipyridin]-6 (1H)-one

To a stirred solution of 4-bromo-5-iodo-1-methylpyridin-2 (1H)-one (3.50 g, 11.2 mmol, 1.00 equiv) and pyridin-3-ylboronic acid (1.64 g, 13.4 mmol, 1.20 equiv) in DMF (35.0 mL) and H₂O (3.5 mL) were added Pd(PPh₃)₄ (1.29 g, 1.11 mmol, 0.10 equiv) and Na₂CO₃ (2.36 g, 22.3 mmol, 2.00 equiv). The resulting mixture was stirred at 100° C. under nitrogen atmosphere for 1 h. The resulting mixture was extracted with EtOAc (3×10 mL). The combined organic layers were washed with brine (2×20 mL), dried over anhydrous Na₂SO₄, filtered and concentrated under reduced pressure. The residue was purified by Prep-HPLC^{CQ} to afford the title compound as a light-yellow solid (1.3 g, 43.9%). LCMS: m/z=267 [M+H]⁺

### Preparation 249: 2-chloro-6-methyl-4-(1-methyl-6-oxo-1,6-dihydro-[3,3′-bipyridin]-4-yl)-1-tosyl-1,6-dihydro-7H-pyrrolo[2,3-c]pyridin-7-one

To a stirred solution of 4-bromo-1-methyl-[3,3′-bipyridin]-6 (1H)-one (1.1 g, 4.15 mmol, 1.00 equiv) and 2-chloro-4-(5,5-dimethyl-1,3,2-dioxaborinan-2-yl)-6-methyl-1-tosyl-1,6-dihydro-7H-pyrrolo[2,3-c]pyridin-7-one (2.23 g, 4.98 mmol, 1.20 equiv) in DME (20.0 mL) and H₂O (5.0 mL) were added Pd(dppf)Cl₂·CH₂Cl₂ (338 mg, 0.415 mmol, 0.100 equiv) and Na₂CO₃ (879 mg, 8.29 mmol, 2.00 equiv). The resulting mixture was stirred at 60° C. under nitrogen atmosphere for 1 h. The residue was purified by Prep-HPLC^{C} to afford the title compound as a light-yellow solid (1.0 g, 46.3%). LCMS: m/z=521 [M+H]⁺

### Preparation 250: 6-methyl-4-(1-methyl-6-oxo-1,6-dihydro-[3,3′-bipyridin]-4-yl)-1-tosyl-2-(1-(trifluoromethyl)-1H-pyrazol-4-yl)-1,6-dihydro-7H-pyrrolo[2,3-c]pyridin-7-on

To the mixture of 2-chloro-6-methyl-4-(1-methyl-6-oxo-1,6-dihydro-[3,3′-bipyridin]-4-yl)-1-tosyl-1,6-dihydro-7H-pyrrolo[2,3-c]pyridin-7-one (900 mg, 1.73 mmol, 1.00 equiv) and 4-(4,4,5,5-tetramethyl-1,3,2-dioxaborolan-2-yl)-1-(trifluoromethyl)pyrazole (905 mg, 3.45 mmol, 2.00 equiv) in H₂O (1.0 mL) and DME (10.0 mL) were added XPhos Pd G3 (146.22 mg, 0.173 mmol, 0.10 equiv) and K₂CO₃ (477.49 mg, 3.454 mmol, 2.00 equiv). The mixture was stirred at 60° C. under nitrogen atmosphere. The residue was purified by Prep-HPLC^{CQ} to afford the title compound as a yellow solid (250 mg, 23.3%). LCMS: m/z=621 [M+H]⁺

### Preparation 251: 6-methyl-4-(1-methyl-6-oxo-1,6-dihydro-[3,3′-bipyridin]-4-yl)-2-(1-(trifluoromethyl)-1H-pyrazol-4-yl)-1,6-dihydro-7H-pyrrolo[2,3-c]pyridin-7-one

To a solution of 6-methyl-4-(1-methyl-6-oxo-1,6-dihydro-[3,3′-bipyridin]-4-yl)-1-tosyl-2-(1-(trifluoromethyl)-1H-pyrazol-4-yl)-1,6-dihydro-7H-pyrrolo[2,3-c]pyridin-7-one (240 mg, 0.387 mmol, 1 equiv) in MeOH (4.80 mL) and H₂O (1.20 mL) was added NaOH (155 mg, 3.87 mmol, 10.0 equiv). The mixture was stirred at 60° C. under nitrogen atmosphere for 1 h. The mixture was concentrated under reduced pressure. The residue was purified by Prep-HPLC^{BW} to afford the title compound as a white solid (100 mg, 55.4%). ¹H NMR (400 MHz, DMSO-d₆) δ 12.31 (s, 1H), 8.88 (s, 1H), 8.42 (dd, J=2.4, 0.9 Hz, 2H), 8.28 (dd, J=4.8, 1.6 Hz, 1H), 8.01 (s, 1H), 7.55 (dt, J=8.0, 1.9 Hz, 1H), 7.22 (s, 1H), 7.21-7.10 (m, 1H), 6.52 (s, 1H), 6.27 (s, 1H), 3.57 (s, 3H), 3.48 (s, 3H). LCMS^{D}: m/z=467 [M+H]⁺

### Example 67 3-fluoro-6-methyl-4-(1-methyl-6-oxo-1,6-dihydro-[3,3′-bipyridin]-4-yl)-2-(1-(trifluoromethyl)-1H-pyrazol-4-yl)-1,6-dihydro-7H-pyrrolo[2,3-c]pyridin-7-one

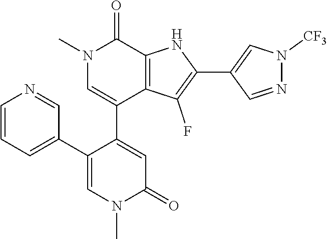 [SMILES: Cn1cc(-c2cccnc2)c(-c2cn(C)c(=O)c3[nH]c(-c4cnn(C(F)(F)F)c4)c(F)c23)cc1=O]

### Preparation 252: 3-fluoro-6-methyl-4-(1-methyl-6-oxo-1,6-dihydro-[3,3′-bipyridin]-4-yl)-2-(1-(trifluoromethyl)-1H-pyrazol-4-yl)-1,6-dihydro-7H-pyrrolo[2,3-c]pyridin-7-one

To a stirred solution of 6-methyl-4-(1-methyl-6-oxo-1,6-dihydro-[3,3′-bipyridin]-4-yl)-2-(1-(trifluoromethyl)-1H-pyrazol-4-yl)-1,6-dihydro-7H-pyrrolo[2,3-c]pyridin-7-one (70.0 mg, 0.150 mmol, 1.00 equiv) in DCM (3.0 mL) was added 1-fluoro-2,6-dichloropyridinium tetrafluoroborate (228 mg, 0.900 mmol, 6.00 equiv). The resulting mixture at room temperature under nitrogen atmosphere was stirred for 3 h. The precipitated solids were collected by filtration and washed with DCM (3×5 mL). The filtered cake was further purified by Prep-HPLC^{CR} to afford the title compound as a light-yellow solid (8.4 mg, 11.55%). ¹H NMR (400 MHz, MeOD) δ 8.42 (m, 2H), 8.26-8.24 (m, 1H), 8.10 (s, 1H), 7.88 (s, 1H), 7.70-7.68 (m, 1H), 7.28 (s, 1H), 7.23-7.20 (m, 1H), 6.72 (s, 1H), 3.71 (s, 3H), 3.63 (s, 3H). LCMS^{BD}: m/z=485 [M+H]⁺

### Example 68 4-(1-(2-hydroxyethyl)-2-oxo-5-phenyl-3-(trifluoromethyl)-1,2-dihydropyridin-4-yl)-6-methyl-2-(1-(trifluoromethyl)-1H-pyrazol-4-yl)-1,6-dihydro-7H-pyrrolo[2,3-c]pyridin-7-one

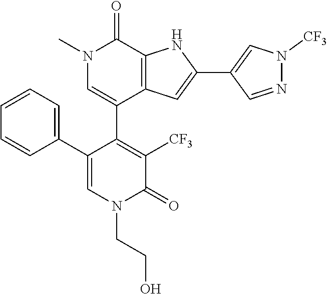 [SMILES: Cn1cc(-c2c(-c3ccccc3)cn(CCO)c(=O)c2C(F)(F)F)c2cc(-c3cnn(C(F)(F)F)c3)[nH]c2c1=O]

### Preparation 253: 2-chloro-4-(1-(2-hydroxyethyl)-2-oxo-5-phenyl-3-(trifluoromethyl)-1,2-dihydropyridin-4-yl)-6-methyl-1-tosyl-1,6-dihydro-7H-pyrrolo[2, 3-c]pyridin-7-one

To a stirred mixture of 4-bromo-1-(2-hydroxyethyl)-5-phenyl-3-(trifluoromethyl) pyridin-2-one (400 mg, 1.15 mmol, 1.00 equiv) and 2-chloro-4-(5,5-dimethyl-1,3,2-dioxaborinan-2-yl)-6-methyl-1-(4-methylbenzenesulfonyl) pyrrolo[2,3-c]pyridine-7-one (743 mg, 1.65 mmol, 1.50 equiv) in DME (5.00 mL) and H₂O (1.00 mL) were added Na₂CO₃ (234 mg, 2.21 mmol, 2.00 equiv) and Pd(dppf)Cl₂—CH₂Cl₂ (89.9 mg, 0.11 mmol, 0.10 equiv). The resulting mixture was stirred at 60° C. under nitrogen atmosphere for 3 h. The resulting mixture was extracted with EtOAc (3×10.0 mL), dried over anhydrous Na₂SO₄, filtered and concentrated under reduced pressure. The residue was purified by silica gel column chromatography (eluted with FE/EA (1:3)) to afford the title compound as a yellow solid (200 mg, crude). LOMS: m/z=618 [M+H]⁺

### Preparation 254: 4-(1-(2-hydroxyethyl)-2-oxo-5-phenyl-3-(trifluoromethyl)-1,2-dihydropyridin-4-yl)-6-methyl-1-tosyl-2-(1-(trifluoromethyl)-1H-pyrazol-4-yl)-1,6-dihydro-7H-pyrrolo[2,3-c]pyridin-7-one

To a stirred mixture of 4-[2-chloro-6-methyl-1-(4-methylbenzenesulfonyl)-7-oxopyrrolo[2,3-c]pyridin-4-yl]-1-(2-hydroxyethyl)-5-phenyl-3-(trifluoromethyl) pyridin-2-one (200 mg, 0.32 mmol, 1.00 equiv) and 4-(4,4,5,5-tetramethyl-1,3,2-dioxaborolan-2-yl)-1-(trifluoromethyl) pyrazole (169 mg, 0.64 mmol, 2.00 equiv) in DME (2.00 mL) and H₂O (0.40 mL) were added K₂CO₃ (89.4 mg, 0.64 mmol, 2.00 equiv) and XPhos Pd G 3 (2.74 mg, 0.03 mmol, 0.10 equiv). The resulting mixture was stirred at 100° C. under nitrogen atmosphere for 2 h. The resulting mixture was extracted with EtOAc (3×10.0 mL), dried over anhydrous Na₂SO₄, filtered and concentrated under reduced pressure. The residue was purified by silica gel column chromatography (eluted with PE/EA (1:3)) to afford the title compound as a yellow solid (100 mg, 43.0%). LCMS: m/z=718 [M+H]⁺

### Preparation 255: 4-(1-(2-hydroxyethyl)-2-oxo-5-phenyl-3-(trifluoromethyl)-1,2-dihydropyridin-4-yl)-6-methyl-2-(1-(trifluoromethyl)-1H-pyrazol-4-yl)-1,6-dihydro-7H-pyrrolo[2,3-c]pyridin-7-one

To a stirred mixture of 4-[6-methyl-1-(4-methylbenzenesulfonyl)-7-oxo-2-[1-(trifluoromethyl) pyrazol-4-yl]pyrrolo[2,3-c]pyridin-4-yl]-5-phenyl-1-[2-(trifluoromethoxy) ethyl]pyridin-2-one (90.0 mg, 0.12 mmol, 1.00 equiv) in MeOH (0.80 mL) and H₂O (0.10 mL) were added NaOH (50.1 mg, 1.25 mmol, 10.0 equiv). The resulting mixture was stirred at 60° C. for 2 h. The resulting mixture was extracted with EtOAc (3×10.0 mL), dried over anhydrous Na₂SO₄, filtered and concentrated under reduced pressure. The crude product (80.0 mg) was purified by Prep-HPLC^{CG} to afford the title compound as a white solid (3.20 mg, 4.53%). ¹H NMR (400 MHz, MeOD) δ 8.56-8.46 (s, 1H), 8.20 (s, 1H), 7.89 (s, 1H), 7.11-7.05 (m, 5H), 6.89 (s, 1H), 6.39 (s, 1H), 4.29-4.22 (m, 2H), 3.95-3.93 (m, 2H), 3.49 (s, 3H). LCMS^{BE}: m/z=564 [M+H]⁺

Examples 69-115 were made according to the General Schemes 5 to 10

| | | m/z |
|---|---|---|
| Structure | Example | [M + H]+ |
| 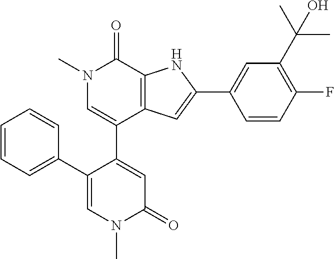 [SMILES: Cn1cc(-c2ccccc2)c(-c2cn(C)c(=O)c3[nH]c(-c4ccc(F)c(C(C)(C)O)c4)cc23)cc1=O] | 69 | 484 |
| 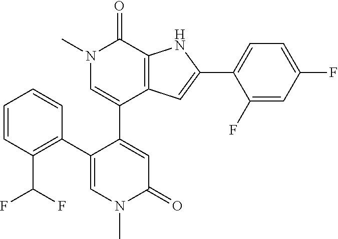 [SMILES: Cn1cc(-c2ccccc2C(F)F)c(-c2cn(C)c(=O)c3[nH]c(-c4ccc(F)cc4F)cc23)cc1=O] | 70 | 494 |
| 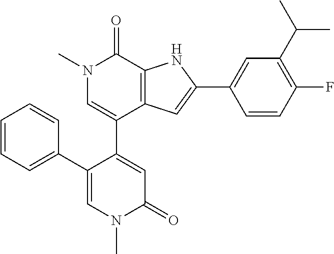 [SMILES: CC(C)c1cc(-c2cc3c(-c4cc(=O)n(C)cc4-c4ccccc4)cn(C)c(=O)c3[nH]2)ccc1F] | 71 | 468 |
| 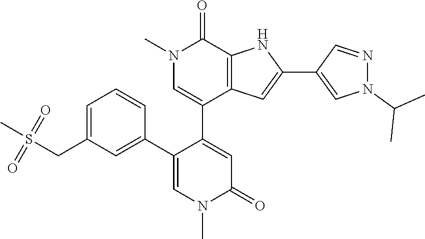 [SMILES: CC(C)n1cc(-c2cc3c(-c4cc(=O)n(C)cc4-c4cccc(CS(C)(=O)=O)c4)cn(C)c(=O)c3[nH]2)cn1] | 72 | 532 |
| 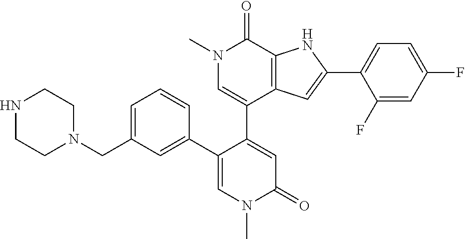 [SMILES: Cn1cc(-c2cccc(CN3CCNCC3)c2)c(-c2cn(C)c(=O)c3[nH]c(-c4ccc(F)cc4F)cc23)cc1=O] | 73 | 542 |
| 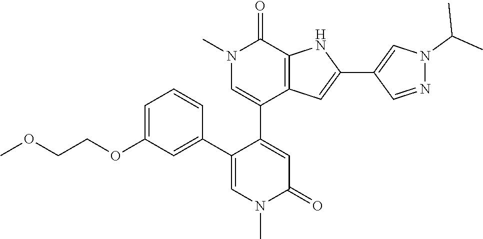 [SMILES: COCCOc1cccc(-c2cn(C)c(=O)cc2-c2cn(C)c(=O)c3[nH]c(-c4cnn(C(C)C)c4)cc23)c1] | 74 | 514 |
| 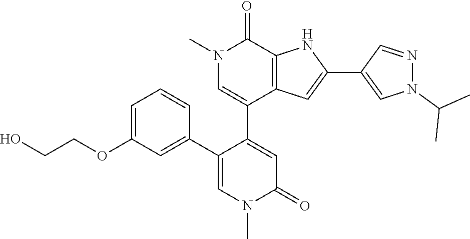 [SMILES: CC(C)n1cc(-c2cc3c(-c4cc(=O)n(C)cc4-c4cccc(OCCO)c4)cn(C)c(=O)c3[nH]2)cn1] | 75 | 500 |
| 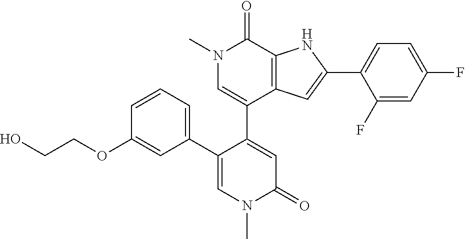 [SMILES: Cn1cc(-c2cccc(OCCO)c2)c(-c2cn(C)c(=O)c3[nH]c(-c4ccc(F)cc4F)cc23)cc1=O] | 76 | 504 |
| 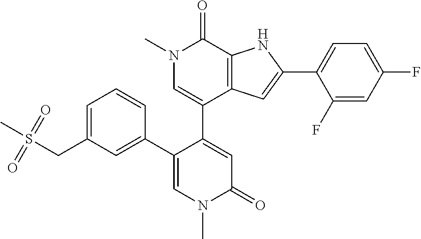 [SMILES: Cn1cc(-c2cccc(CS(C)(=O)=O)c2)c(-c2cn(C)c(=O)c3[nH]c(-c4ccc(F)cc4F)cc23)cc1=O] | 77 | 536 |
| 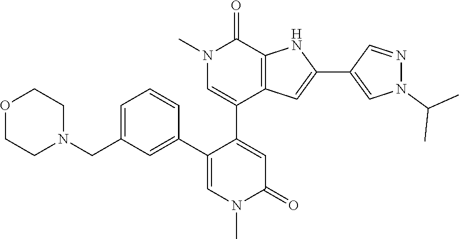 [SMILES: CC(C)n1cc(-c2cc3c(-c4cc(=O)n(C)cc4-c4cccc(CN5CCOCC5)c4)cn(C)c(=O)c3[nH]2)cn1] | 78 | 539 |
| 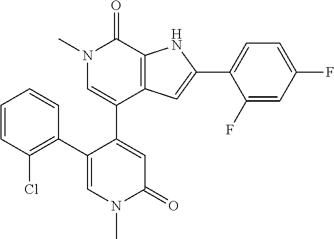 [SMILES: Cn1cc(-c2ccccc2Cl)c(-c2cn(C)c(=O)c3[nH]c(-c4ccc(F)cc4F)cc23)cc1=O] | 79 | 478 |
| 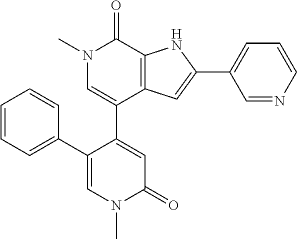 [SMILES: Cn1cc(-c2ccccc2)c(-c2cn(C)c(=O)c3[nH]c(-c4cccnc4)cc23)cc1=O] | 80 | 409 |
| 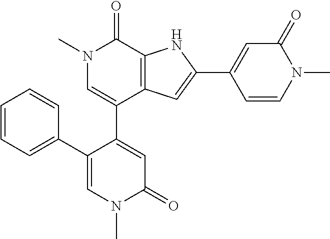 [SMILES: Cn1ccc(-c2cc3c(-c4cc(=O)n(C)cc4-c4ccccc4)cn(C)c(=O)c3[nH]2)cc1=O] | 81 | 439 |
| 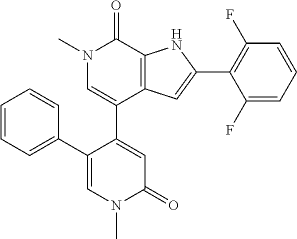 [SMILES: Cn1cc(-c2ccccc2)c(-c2cn(C)c(=O)c3[nH]c(-c4c(F)cccc4F)cc23)cc1=O] | 82 | 444 |
| 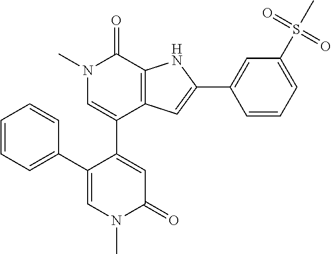 [SMILES: Cn1cc(-c2ccccc2)c(-c2cn(C)c(=O)c3[nH]c(-c4cccc(S(C)(=O)=O)c4)cc23)cc1=O] | 83 | 486 |
| 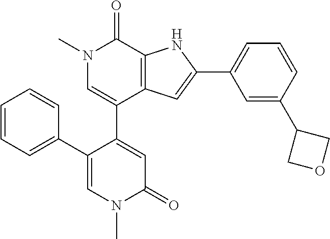 [SMILES: Cn1cc(-c2ccccc2)c(-c2cn(C)c(=O)c3[nH]c(-c4cccc(C5COC5)c4)cc23)cc1=O] | 84 | 464 |
| 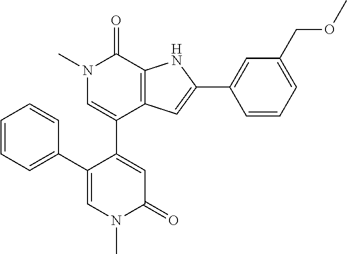 [SMILES: COCc1cccc(-c2cc3c(-c4cc(=O)n(C)cc4-c4ccccc4)cn(C)c(=O)c3[nH]2)c1] | 85 | 452 |
| 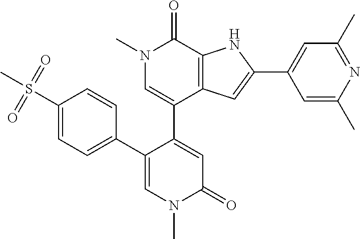 | 86 | 515 |
| 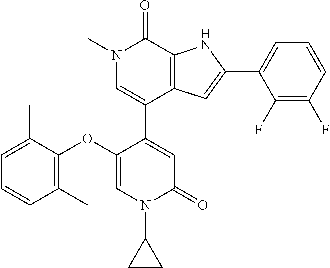 [SMILES: Cc1cccc(C)c1Oc1cn(C2CC2)c(=O)cc1-c1cn(C)c(=O)c2[nH]c(-c3cccc(F)c3F)cc12] | 87 | 514 |
| 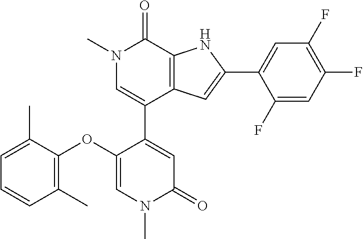 [SMILES: Cc1cccc(C)c1Oc1cn(C)c(=O)cc1-c1cn(C)c(=O)c2[nH]c(-c3cc(F)c(F)cc3F)cc12] | 88 | 506 |
| 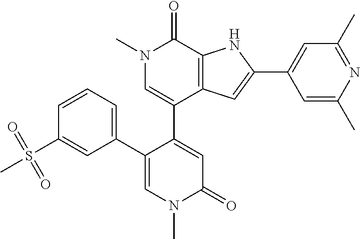 | 89 | 515 |
| 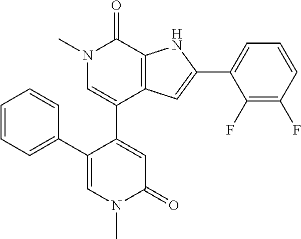 [SMILES: Cn1cc(-c2ccccc2)c(-c2cn(C)c(=O)c3[nH]c(-c4cccc(F)c4F)cc23)cc1=O] | 90 | 444 |
| 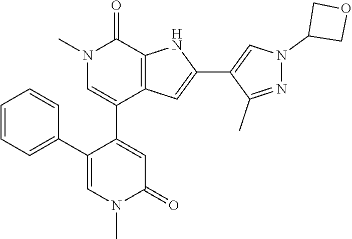 [SMILES: Cc1nn(C2COC2)cc1-c1cc2c(-c3cc(=O)n(C)cc3-c3ccccc3)cn(C)c(=O)c2[nH]1] | 91 | 468 |
| 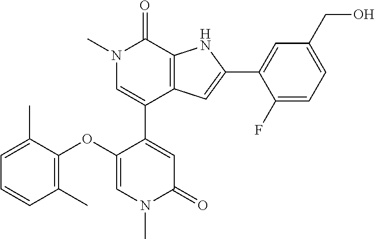 [SMILES: Cc1cccc(C)c1Oc1cn(C)c(=O)cc1-c1cn(C)c(=O)c2[nH]c(-c3cc(CO)ccc3F)cc12] | 92 | 500 |
| 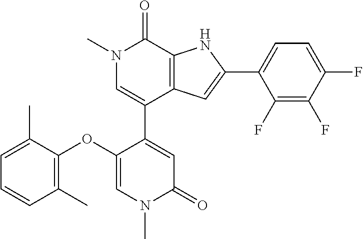 [SMILES: Cc1cccc(C)c1Oc1cn(C)c(=O)cc1-c1cn(C)c(=O)c2[nH]c(-c3ccc(F)c(F)c3F)cc12] | 93 | 506 |
| 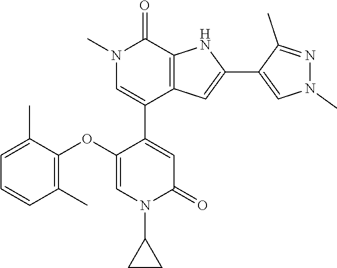 [SMILES: Cc1cccc(C)c1Oc1cn(C2CC2)c(=O)cc1-c1cn(C)c(=O)c2[nH]c(-c3cn(C)nc3C)cc12] | 94 | 496 |
| 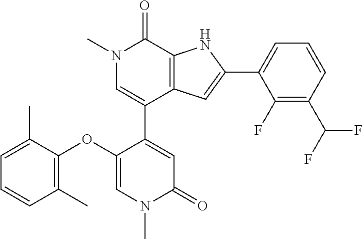 [SMILES: Cc1cccc(C)c1Oc1cn(C)c(=O)cc1-c1cn(C)c(=O)c2[nH]c(-c3cccc(C(F)F)c3F)cc12] | 95 | 520 |
| 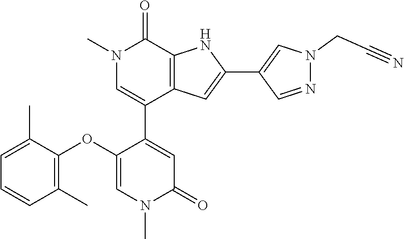 [SMILES: Cc1cccc(C)c1Oc1cn(C)c(=O)cc1-c1cn(C)c(=O)c2[nH]c(-c3cnn(CC#N)c3)cc12] | 96 | 481 |
| 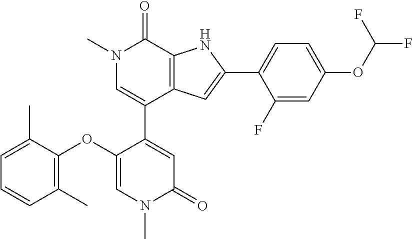 [SMILES: Cc1cccc(C)c1Oc1cn(C)c(=O)cc1-c1cn(C)c(=O)c2[nH]c(-c3ccc(OC(F)F)cc3F)cc12] | 97 | 536 |
| 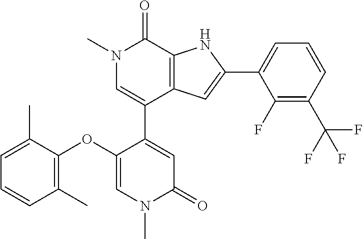 [SMILES: Cc1cccc(C)c1Oc1cn(C)c(=O)cc1-c1cn(C)c(=O)c2[nH]c(-c3cccc(C(F)(F)F)c3F)cc12] | 98 | 538 |
| 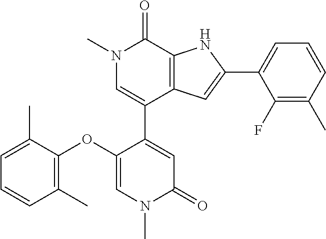 [SMILES: Cc1cccc(-c2cc3c(-c4cc(=O)n(C)cc4Oc4c(C)cccc4C)cn(C)c(=O)c3[nH]2)c1F] | 99 | 484 |
| 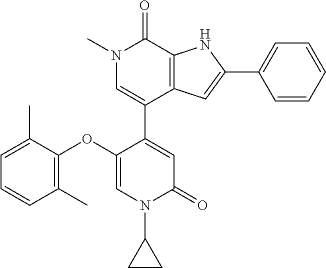 [SMILES: Cc1cccc(C)c1Oc1cn(C2CC2)c(=O)cc1-c1cn(C)c(=O)c2[nH]c(-c3ccccc3)cc12] | 100 | 478 |
| 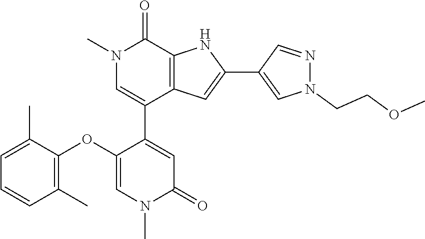 [SMILES: COCCn1cc(-c2cc3c(-c4cc(=O)n(C)cc4Oc4c(C)cccc4C)cn(C)c(=O)c3[nH]2)cn1] | 101 | 500 |
| 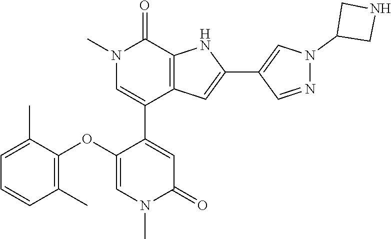 [SMILES: Cc1cccc(C)c1Oc1cn(C)c(=O)cc1-c1cn(C)c(=O)c2[nH]c(-c3cnn(C4CNC4)c3)cc12] | 102 | 497 |
| 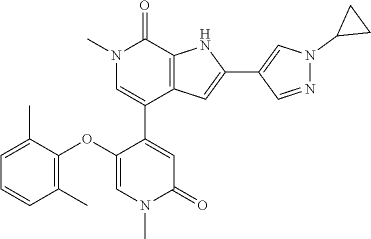 [SMILES: Cc1cccc(C)c1Oc1cn(C)c(=O)cc1-c1cn(C)c(=O)c2[nH]c(-c3cnn(C4CC4)c3)cc12] | 103 | 482 |
| 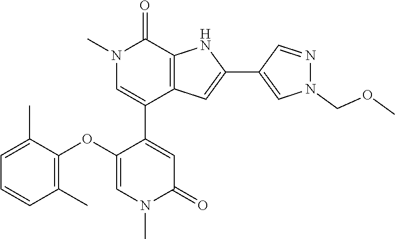 [SMILES: COCn1cc(-c2cc3c(-c4cc(=O)n(C)cc4Oc4c(C)cccc4C)cn(C)c(=O)c3[nH]2)cn1] | 104 | 486 |
| 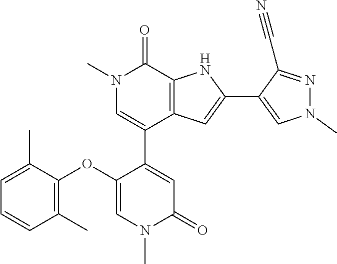 [SMILES: Cc1cccc(C)c1Oc1cn(C)c(=O)cc1-c1cn(C)c(=O)c2[nH]c(-c3cn(C)nc3C#N)cc12] | 105 | 481 |
| 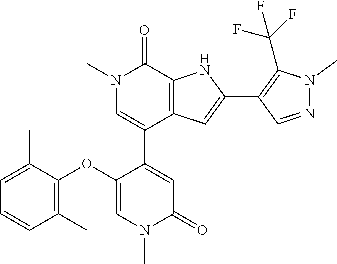 [SMILES: Cc1cccc(C)c1Oc1cn(C)c(=O)cc1-c1cn(C)c(=O)c2[nH]c(-c3cnn(C)c3C(F)(F)F)cc12] | 106 | 524 |
| 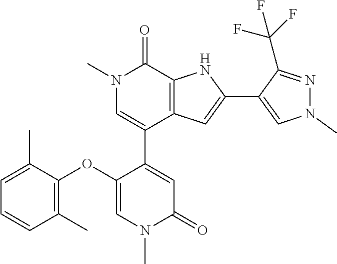 [SMILES: Cc1cccc(C)c1Oc1cn(C)c(=O)cc1-c1cn(C)c(=O)c2[nH]c(-c3cn(C)nc3C(F)(F)F)cc12] | 107 | 524 |
| 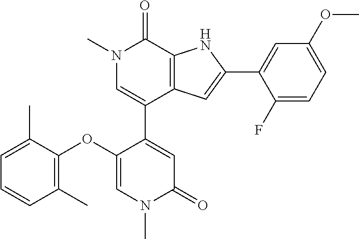 [SMILES: COc1ccc(F)c(-c2cc3c(-c4cc(=O)n(C)cc4Oc4c(C)cccc4C)cn(C)c(=O)c3[nH]2)c1] | 108 | 500 |
| 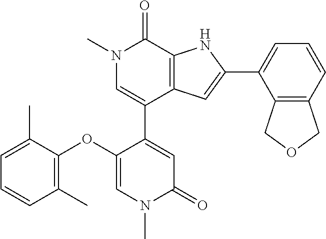 [SMILES: Cc1cccc(C)c1Oc1cn(C)c(=O)cc1-c1cn(C)c(=O)c2[nH]c(-c3cccc4c3COC4)cc12] | 109 | 494 |
| 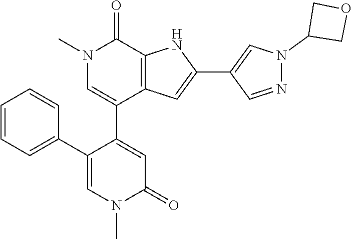 [SMILES: Cn1cc(-c2ccccc2)c(-c2cn(C)c(=O)c3[nH]c(-c4cnn(C5COC5)c4)cc23)cc1=O] | 110 | 454 |
| 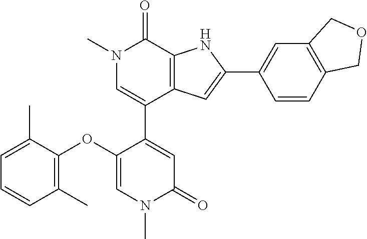 [SMILES: Cc1cccc(C)c1Oc1cn(C)c(=O)cc1-c1cn(C)c(=O)c2[nH]c(-c3ccc4c(c3)COC4)cc12] | 111 | 494 |
| 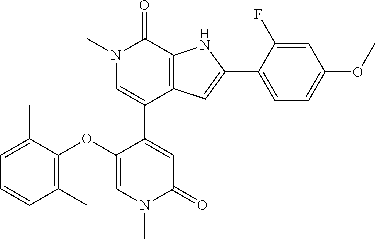 [SMILES: COc1ccc(-c2cc3c(-c4cc(=O)n(C)cc4Oc4c(C)cccc4C)cn(C)c(=O)c3[nH]2)c(F)c1] | 112 | 500 |
| 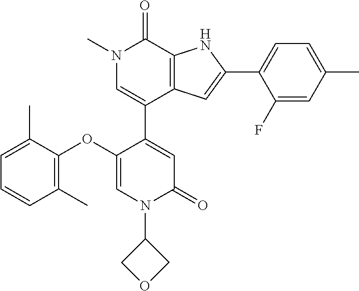 [SMILES: Cc1ccc(-c2cc3c(-c4cc(=O)n(C5COC5)cc4Oc4c(C)cccc4C)cn(C)c(=O)c3[nH]2)c(F)c1] | 113 | 526 |
| 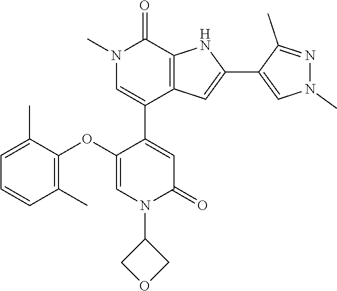 [SMILES: Cc1cccc(C)c1Oc1cn(C2COC2)c(=O)cc1-c1cn(C)c(=O)c2[nH]c(-c3cn(C)nc3C)cc12] | 114 | 512 |
| 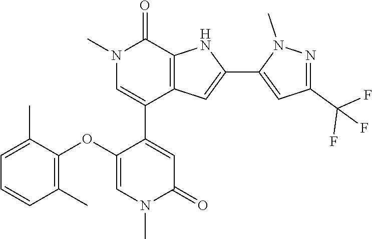 [SMILES: Cc1cccc(C)c1Oc1cn(C)c(=O)cc1-c1cn(C)c(=O)c2[nH]c(-c3cc(C(F)(F)F)nn3C)cc12] | 115 | 524 |

### Example 116 4-{3-chloro-6-methyl-7-oxo-2-[1-(trifluoromethyl)pyrazol-4-yl]-1H-pyrrolo[2,3-c]pyridin-4-yl}-1-methyl-[3,3′-bipyridin]-6-one

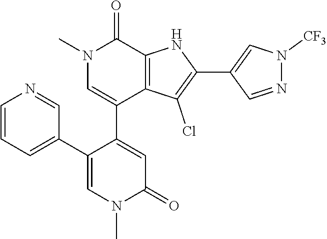 [SMILES: Cn1cc(-c2cccnc2)c(-c2cn(C)c(=O)c3[nH]c(-c4cnn(C(F)(F)F)c4)c(Cl)c23)cc1=O]

### Preparation 256: 4-{3-chloro-6-methyl-7-oxo-2-[1-(trifluoromethyl)pyrazol-4-yl]-1H-pyrrolo[2,3-c]pyridin-4-yl}-1-methyl-[3,3′-bipyridin]-6-one

To a stirred solution of 1-methyl-4-{6-methyl-7-oxo-2-[1-(trifluoromethyl)pyrazol-4-yl]-1H-pyrrolo[2,3-c]pyridin-4-yl}-[3,3′-bipyridin]-6-one (320 mg, 0.686 mmol, 1.00 equiv) in DCM (15 mL) at room temperature was added 2,6-dichloro-1-fluoropyridin-1-ium tetrafluoroborate (1.04 g, 4.11 mmol, 6.00 equiv). The resulting mixture was stirred for 4 h at room temperature. The resulting mixture was concentrated under reduced pressure. The crude product was purified by Prep Chiral-HPLC^{CS} to afford 3-fluoro-6-methyl-4-(1-methyl-6-oxo-1,6-dihydro-[3,3′-bipyridin]-4-yl)-2-(1-(trifluoromethyl)-1H-pyrazol-4-yl)-1,6-dihydro-7H-pyrrolo[2,3-c]pyridin-7-one (35 mg, 10.5%) as a white solid and 4-{3-chloro-6-methyl-7-oxo-2-[1-(trifluoromethyl)pyrazol-4-yl]-1H-pyrrolo[2,3-c]pyridin-4-yl}-1-methyl-[3,3′-bipyridin]-6-one (4.9 mg, 1.43%) as a white solid. ¹H NMR (400 MHz, MeOD) δ 8.60 (s, 1H), 8.41 (s, 1H), 8.29 (s, 1H), 8.26-8.25 (m, 1H), 7.86 (s, 1H), 7.69-7.67 (m, 1H), 7.27 (s, 1H), 7.22-7.19 (m, 1H), 6.69 (s, 1H), 3.71 (s, 3H), 3.63 (s, 3H. LCMS^{AT}: m/z=501 [M+H]⁺

### Primary Activity

Exemplary compounds of the disclosure are active against BRD4 BD2 and selective over BRD4 BD1. BRD4 is a representative example of the BET family, as the binding sites of all BET family members are structurally similar. The half maximal inhibitory concentration (IC50) of Examples 1 to 116 of the compounds is described herein against BRD4 BD1 and BD2 and the fold selectivity calculated (IC50 BD1/IC50 BD2). IC50s and fold selectivity's are determined as described below and are represented in Table 1.

### Bromodomain Assay Procedure

NanoBRET assay was carried out according to the manufacturer's suggested protocol (Promega, Madison, WI). HEK293 cells were transfected using NanoLuc-BRD4-BD1 or NanoLuc-BRD4-BD2 fusion vectors and incubated at 37° C. in an atmosphere of 5% CO₂ for 20-24 hours. The transfected cells were then dispensed into 96-well plates using 90 μl cell suspension per well containing 2×10⁵ cells/ml in Opti-MEM and 1× final concentration of tracer. 90 μl per well of cell suspension without tracer was also dispensed into at least 3 wells as “No tracer control samples” for background correction. Serially diluted test compounds were prepared at 1000× concentration in DMSO and further diluted to 1OX concentration in Opti-MEM. 10 μl per well of the serially diluted 1OX test compound was added to the 96-well plates containing cells with 1× tracer. Plates were then incubated at 37° C.+5% CO2 incubator for 2 hours. Immediately prior to BRET measurements, a 3× solution consisting of 1:166 dilution of Nano-Glo® Substrate plus a 1:500 dilution of Extracellular NanoLuc Inhibitor in Opti-MEM was prepared and 50 μl per well was added to the cells. Donor emission (450 nm) and acceptor emission (610 nm) were measured using PHERAstar (BMG LabTech). For data analysis, the raw BRET ratio was generated and converted to milliBRET units with background correction using the formula: [(Acceptorₛₐₘₚₗₑ/Donorₛₐₘₚₗₑ)−(Acceptor_{no tracer control}/Donor_{no tracer control})]×1000. The mBU data was plotted as a function of compound concentration and IC50s for BRET assay were determined by nonlinear regression analysis of concentration response curves using the GraphPad Prism software.

### Fold Selectivity Calculated (IC50 BD1/IC50 BD2).

TABLE 1
IC50s of Examples 1-116 from BRD4 BD1 and BD2

| | | | Fold |
|---|---|---|---|
| | | | Selectivity |
| | BRD4 BD1 | BRD4 BD2 | BRD4 |
| Example | IC₅₀ | IC₅₀ | BD1/BD2 |
| 1 | # | ++++ | xxx |
| 2 | # | +++ | xx |
| 3 | # | +++ | xx |
| 4 | # | + | — |
| 5 | # | ++++ | xxx |
| 6 | # | +++++ | xxxx |
| 7 | # | +++++ | xxxx |
| 8 | # | +++++ | xxxx |
| 9 | # | +++++ | xxxx |
| 10 | # | ++++ | xxx |
| 11 | # | +++++ | xxxx |
| 12 | # | ++++ | xxx |
| 13 | # | ++++ | xxx |
| 14 | ### | +++++ | x |
| 15 | ## | ++++ | xx |
| 16 | # | +++++ | xx |
| 17 | # | +++++ | xxxx |
| 18 | # | +++++ | xxxx |
| 19 | # | +++++ | xxxx |
| 20 | # | ++++ | xxx |
| 21 | # | +++++ | xxxx |
| 22 | # | +++++ | xxxx |
| 23 | # | +++++ | xxxx |
| 24 | # | +++++ | xxxx |
| 25 | # | +++++ | xxxx |
| 26 | ## | +++++ | xxx |
| 27 | # | ++++ | xxx |
| 28 | # | +++++ | xxxx |
| 29 | # | ++++ | xxx |
| 30 | # | +++++ | xxxx |
| 31 | ## | +++++ | xxx |
| 32 | # | ++++ | xxx |
| 33 | # | +++++ | xxxx |
| 34 | # | +++++ | xxxx |
| 35 | # | ++++ | xxx |
| 36 | # | ++++ | xxx |
| 37 | # | ++++ | xxx |
| 38 | # | ++++ | xxx |
| 39 | # | +++++ | xxxx |
| 40 | # | ++++ | xxx |
| 41 | # | +++++ | xxxx |
| 42 | # | +++++ | xxxx |
| 43 | # | +++++ | xxxx |
| 44 | # | +++++ | xxxx |
| 45 | # | +++++ | xxxx |
| 46 | # | +++++ | xxxx |
| 47 | # | +++++ | xxxx |
| 48 | # | +++++ | xxxx |
| 49 | # | +++++ | xxxx |
| 50 | # | +++++ | xxxx |
| 51 | # | +++++ | xxxx |
| 52 | # | +++++ | xxxx |
| 53 | # | +++++ | xxxx |
| 54 | # | +++++ | xxxx |
| 55 | # | +++++ | xxxx |
| 56 | # | +++++ | xxxx |
| 57 | # | +++++ | xxxx |
| 58 | # | +++++ | xxxx |
| 59 | # | +++ | xx |
| 60 | # | ++++ | xxx |
| 61 | ### | +++++ | x |
| 62 | # | +++++ | xxxx |
| 63 | ## | +++++ | xx |
| 64 | ## | +++ | x |
| 65 | # | ++++ | xxx |
| 66 | # | ++ | x |
| 67 | # | ++++ | xxx |
| 68 | # | ++ | x |
| 69 | ## | +++++ | xxx |
| 70 | # | ++ | x |
| 71 | # | +++ | xx |
| 72 | # | ++ | x |
| 73 | # | ++ | x |
| 74 | # | ++++ | xx |
| 75 | # | ++ | x |
| 76 | # | ++++ | xxx |
| 77 | # | +++ | xx |
| 78 | # | ++++ | xxx |
| 79 | # | ++++ | xxx |
| 80 | ### | +++++ | xx |
| 81 | # | +++ | xx |
| 82 | # | +++ | xx |
| 83 | ## | +++++ | xx |
| 84 | ### | +++++ | xx |
| 85 | # | +++++ | xxxx |
| 86 | # | ++ | x |
| 87 | # | + | — |
| 88 | # | ++++ | xxx |
| 89 | # | +++ | xx |
| 90 | # | ++++ | xxx |
| 91 | # | ++++ | xx |
| 92 | # | ++++ | xxx |
| 93 | # | +++ | xx |
| 94 | # | ++++ | xxx |
| 95 | # | +++ | xx |
| 96 | ## | ++++ | x |
| 97 | # | ++++ | xxx |
| 98 | # | +++ | xx |
| 99 | # | ++++ | xxx |
| 100 | # | ++++ | xxx |
| 101 | # | ++++ | xxx |
| 102 | # | ++ | x |
| 103 | # | +++++ | xxx |
| 104 | # | +++++ | xx |
| 105 | ## | +++++ | xx |
| 106 | # | ++++ | xxx |
| 107 | # | +++ | xx |
| 108 | # | ++++ | xxx |
| 109 | # | + | — |
| 110 | ### | ++++ | x |
| 111 | # | ++++ | xxx |
| 112 | # | ++++ | xxx |
| 113 | # | + | — |
| 114 | # | ++++ | xxx |
| 115 | # | +++ | xx |
| 116 | # | ++ | x |

Note:
If IC₅₀ of one or both BD2 and BD1 are at the upper limit then selectivity is qualitative. IC₅₀ numbers used to determine banding were rounded to nearest integer.
Key
+ BRD4 BD2 IC50 >1000 nM
++ BRD4 BD2 IC50 >200 nM and ≤1000 nM
+++ BRD4 BD2 IC50 >50 nM and ≤200 nM
++++ BRD4 BD2 IC50 >10 nM and ≤50 nM
+++++ BRD4 BD2 IC50 ≤10 nM
# BRD4 BD1 IC50 >1000 nM
## BRD4 BD1 IC50 >500 nM and ≤1000 nM
### BRD4 BD1 IC50 >50 nM and ≤500 nM
— Fold ≥0 and ≤2
x Fold >2 and ≤50
xx Fold >50 and ≤200
xxx Fold >200 and ≤1000
xxxx Fold >1000

### Experimental Method A: Preparation of Formulations used in Methods B, C and D

### Compound Formulations

TABLE 2A
Compound formulations - where Compound A is provided in a propylene glycol based formulation, e.g.,
for Dog PK study #1, for CIA study, for UUO study, and for Rat PK study #1 and Rat PK study #4.

| | | Dose | | | |
|---|---|---|---|---|---|
| | | Level | | Concentration | |
| Compound | Study | (mg/kg) | Preparation | (mg/mL) | Frequency |
| Vehicle | Rat #1 | — | 20% propylene glycol/20% Vitamin E TPGS/60% water | — | RT |
| | Rat #4 | | | | |
| | CIA | | | | |
| | UUO | | | | |
| Dexamethasone | CIA | 0.1 | Saline E.g., dissolve 0.89 mg of Dexamethasone | 0.02 | Prepared fresh |
| | | | in 43.165 mL of saline and vortex | | daily |
| GSK620 | CIA | 10 | 1% MC E.g., dissolve 18.73 mg of GSK620 in 9.271 | 2 | No need to prepare |
| | | | mL of 1% methylcellulose and vortex | | fresh daily |
| Compound A | CIA | 1 | 20% propylene glycol/20% Vitamin E TPGS/60% water | 0.1 | |
| | | | E.g., dissolve 2.08 mg of Compound A in 4.118 mL | | |
| | | | of propylene glycol, then add 4.118 mL of Vitamin E | | |
| | | | TPGS, and finally add 12.356 mL of water and vortex | | |
| | | | to obtain the solution | | |
| Compound A | Rat #4 | 0.2 | 20% propylene glycol/20% Vitamin E TPGS/60% water | 0.04 | |
| | | | E.g., dissolve 0.63 mg of Compound A in 3.150 mL of | | |
| | | | propylene glycol with vortex and sonication, then | | |
| | | | added 3.150 mL of Vitamin E TPGS with vortex and | | |
| | | | sonication, then added 9.450 mL of water with | | |
| | | | vortex to obtain the solution. | | |
| Compound A | Rat #4 | 1 | 20% propylene glycol/20% Vitamin E TPGS/60% water | 0.2 | |
| | | | E.g., dissolve 1.15 mg of Compound A in 1.150 mL of | | |
| | | | propylene glycol with vortex and sonication, then | | |
| | | | add 1.150 mL of Vitamin E TPGS with vortex and | | |
| | | | sonication, finally add 3.450 mL of water and vortex | | |
| | | | to obtain the solution | | |
| Compound A | CIA | 3 | 20% propylene glycol/20% Vitamin E TPGS/60% water | 0.3 | |
| | | | E.g., dissolve 10.72 mg of Compound A in 7.075 mL of | | |
| | | | propylene glycol, then add 7.075 mL of Vitamin E | | |
| | | | TPGD, and finally add 21.226 mL of water and | | |
| | | | vortex to obtain the solution | | |
| Compound A | Dog #1 | 1 | 20% propylene glycol/20% Vitamin E TPGS/60% water | 0.5 | |
| | | | E.g., for IV administration, dissolve 38.45 mg of | | |
| | | | Compound A in 15.380 mL of propylene glycol with | | |
| | | | vortex and sonication, then added 15.380 mL of | | |
| | | | Vitamin E TPGS with vortex and sonication, then | | |
| | | | added 46.140 mL of water with vortex and heat | | |
| | | | to obtain the solution. | | |
| Compound A | Dog# 1 | 5 | 20% propylene glycol/20% Vitamin E TPGS/60% water | 1 | |
| | | | E.g., for PO (oral) administration) dissolve 191.92 | | |
| | | | mg of Compound A in 38.384 mL of propylene | | |
| | | | glycol with vortex and sonication, then added | | |
| | | | 38.384 mL of Vitamin E TPGS with vortex and | | |
| | | | sonication, then added 115.152 mL of water with | | |
| | | | vortex and heat to obtain the solution. | | |
| Compound A | CIA | 10 | 20% propylene glycol/20% Vitamin E TPGS/60% water | 1 | |
| | | | E.g., dissolve 18.87 mg of Compound A in | | |
| | | | 3.736 mL of propylene glycol with vortex and | | |
| | | | sonication, then add 3.736 mL of Vitamin E | | |
| | | | TPGS with vortex and sonification at 50° C., | | |
| | | | finally add 11.209 mL of water and vortex | | |
| | | | to obtain the solution. | | |
| Compound A | UUO | 10 | 20% propylene glycol/20% Vitamin E TPGS/60% water | 1 | Use stock |
| | | | E.g., dissolve 404 mg of Compound A in 80 mL in | | solution |
| | | | propylene glycol with vortex and sonication firstly, | | |
| | | | then added 80 mL of Vitamin E TPGS with vortex | | |
| | | | and sonication, finally added 240 mL of water and | | |
| | | | heat to 40-50° C. to obtain solution. Divided | | |
| | | | into 14 aliquots and store at −20° C. | | |

TABLE 2B
Compound formulations where Compound A is provided in a HPβCD
based formulation, e.g., for Dog PK study #2

| | Dose Level | | Concentration |
|---|---|---|---|
| Compound | (mg/kg) | Preparation | (mg/mL) |
| Compound | 1 | HPβCD (20% solution | 0.5 |
| A | | w/v /DMSO (99/1) | |
| | | E.g., 41.16 mg of | |
| | | Compound A was | |
| | | dissolved in 0.823 mL of | |
| | | DMSO with vortex and | |
| | | sonication and then | |
| | | 81.497 mL of HPβCD | |
| | | (20% solution w/v) was | |
| | | added with vortexing. | |

TABLE 2C
Compound formulations where Compound A is
provided in a methylcellulose-based formulation,
e.g., for Rat PK study #1.

| | | Concentration |
|---|---|---|
| Treatment | Preparation | (mg/mL) |
| Vehicle | 1% methylcellulose | — |
| Compound A | 1% methylcellulose | 1 |
| | G | |
| | E.g., add 9.88 mg of Compound A | |
| | in 9.880 mL of 1% methylcellulose | |
| | (400 cp) in water with vortex and | |
| | sonification to obtain a suspension. | |

TABLE 2D
Compound formulations where Compound A is provided in
a 5% DMSO/40% PEG-400, and 55% Milli-Q Water formulation,
e.g., for Rat PK study #2 and Rat PK study #3.

| | | Concentration |
|---|---|---|
| Treatment | Preparation | (mg/mL) |
| Vehicle | 5% DMSO, 40% PEG-400, 55% Milli-Q water | — |
| ABBV-744 | 5% DMSO, 40% PEG-400, 55% Milli-Q water | 0.2 |
| | E.g., dissolve 1.11 mg of ABBV472 in 0.278 mL of | |
| | DMSO with vortex and sonication, then add 2.220 mL | |
| | of PEG-400 with vortex and sonication, finally add | |
| | 3.053 mL of Milli-Q water with vortex to obtain the | |
| | solution. | |
| ABBV-744 | 5% DMSO, 40% PEG-400, 55% Milli-Q water | 0.5 |
| | E.g., dissolve 5.11 mg of ABBV472 in 0.511 mL of | |
| | DMSO with vortex and sonication, then add 4.088 mL | |
| | of PEG-400 with vortex and sonication, finally add | |
| | 5.621 mL of Milli-Q water with vortex to obtain the | |
| | solution. | |
| Compound A | 5% DMSO, 40% PEG-400, 55% Milli-Q water | 0.5 |
| | E.g., dissolve 4.99 mg of Compound A in 0.499 mL of | |
| | DMSO with vortex and sonication, then add 3.992 ml | |
| | of PEG-400 with vortex and sonication, finally add | |
| | 5.489 mL of Milli-Q water with vortex to obtain the | |
| | solution | |

TABLE 2E
Compound formulations where Compound A is provided in a 5%
DMSO/15% PEG-400/80% (10% VE-TPGS) formulation, e.g., for
Dog PK study #3, Dog PK study #4, SLE study and EAE study.

| | | Nominal | |
|---|---|---|---|
| | | Dose | Concentration |
| Treatment | Preparation | mg/kg | (mg/mL) |
| Vehicle | 5% DMSO/15% PEG-400/80% (10% | | |
| | VE-TPGS) | | |
| Compound | 5% DMSO/15% PEG-400/80% (10% | 1 | 0.2 |
| A | VE-TPGS) | | |
| | E.g., dissolve 1.996 mg of Compound A | | |
| | in 0.499 mL of DMSO with vortex and | | |
| | sonication, then add 1.497 mL of PEG- | | |
| | 400 with vortex and sonication, finally | | |
| | add 7.984 mL of 10% Vitamin E TPGS | | |
| | with vortex to obtain the solution. | | |
| Compound | 5% DMSO/15% PEG-400/80% (10% | 3 | 0.6 |
| A | VE-TPGS) | | |
| | E.g., dissolve 5.988 mg of Compound A | | |
| | in 0.499 mL of DMSO with vortex and | | |
| | sonication, then add 1.497 mL of PEG- | | |
| | 400 with vortex and sonication, finally | | |
| | add 7.984 mL of 10% Vitamin E TPGS | | |
| | with vortex to obtain the solution. | | |
| Compound | 5% DMSO/15% PEG-400/80% (10% | 5 | 1.0 |
| A | VE-TPGS) | | |
| | E.g., dissolve 9.98 mg of Compound A | | |
| | in 0.499 mL of DMSO with vortex and | | |
| | sonication, then add 1.497 mL of PEG- | | |
| | 400 with vortex and sonication, finally | | |
| | add 7.984 mL of 10% Vitamin E TPGS | | |
| | with vortex to obtain the solution. | | |
| Compound | 5% DMSO/15% PEG-400/80% (10% | 10 | 2.0 |
| A | VE-TPGS) | | |
| | E.g., dissolve 19.96 mg of Compound | | |
| | A in 0.499 mL of DMSO with vortex | | |
| | and sonication, then add 1.497 mL of | | |
| | PEG-400 with vortex and sonication, | | |
| | finally add 7.984 mL of 10% Vitamin E | | |
| | TPGS with vortex to obtain the | | |
| | solution. | | |
| Compound | 5% DMSO/15% PEG-400/80% (10% | 20 | 4.0 |
| A | VE-TPGS) | | |
| | E.g., dissolve 39.92 mg of Compound | | |
| | A in 0.499 mL of DMSO with vortex | | |
| | and sonication, then add 1.497 mL of | | |
| | PEG-400 with vortex and sonication, | | |
| | finally add 7.984 mL of 10% Vitamin E | | |
| | TPGS with vortex to obtain the | | |
| | solution. | | |

TABLE 2F
Compound formulations where Compound A and Compound
B are formulated in a 5% DMSO/40% PEG-400/55% water
(0.9% NaCl) formulation, e.g., for psoriasis study.

| | | | Concen- |
|---|---|---|---|
| | | Dose | tration |
| Treatment | Preparation | mg/kg | (mg/mL) |
| Vehicle | 5% DMSO/40% PEG-400/55% | | |
| | water (0.9% NaCl) | | |
| Compound A | 5% DMSO/40% PEG-400/55% | 1 | 0.4 |
| | water (0.9% NaCl) | | |
| | E.g., dissolve 3.992 mg of | | |
| | Compound A in 0.499 mL of | | |
| | DMSO with vortex and | | |
| | sonication, then add 3.992 mL of | | |
| | PEG-400 with vortex and | | |
| | sonication, finally add 5.489 mL | | |
| | of water (0.9% NaCl) with vortex | | |
| | to obtain the solution. | | |
| Compound A | 5% DMSO/40% PEG-400/55% | 3 | 1.2 |
| | water (0.9% NaCl) | | |
| | E.g., dissolve 11.98 mg of | | |
| | Compound A in 0.499 mL of | | |
| | DMSO with vortex and | | |
| | sonication, then add 3.992 mL of | | |
| | PEG-400 with vortex and | | |
| | sonication, finally add 5.489 mL | | |
| | of water (0.9% NaCl) with vortex | | |
| | to obtain the solution. | | |
| Compound A | 5% DMSO/40% PEG-400/55% | 10 | 4 |
| | water (0.9% NaCl) | | |
| | E.g., dissolve 39.92 mg of | | |
| | Compound A in 0.499 mL of | | |
| | DMSO with vortex and | | |
| | sonication, then add 3.992 mL of | | |
| | PEG-400 with vortex and | | |
| | sonication, finally add 5.489 mL | | |
| | of water (0.9% NaCl) with vortex | | |
| | to obtain the solution. | | |
| Compound B | 5% DMSO/40% PEG-400/55% | 1 | 0.4 |
| | water (0.9% NaCl) | | |
| | E.g., dissolve 3.992 mg of | | |
| | Compound A in 0.499 mL of | | |
| | DMSO with vortex and | | |
| | sonication, then add 3.992 mL of | | |
| | PEG-400 with vortex and | | |
| | sonication, finally add 5.489 mL | | |
| | of water (0.9% NaCl) with vortex | | |
| | to obtain the solution. | | |
| Compound B | 5% DMSO/40% PEG-400/55% | 3 | 1.2 |
| | water (0.9% NaCl) | | |
| | E.g., dissolve 11.98 mg of | | |
| | Compound A in 0.499 mL of | | |
| | DMSO with vortex and | | |
| | sonication, then add 3.992 mL of | | |
| | PEG-400 with vortex and | | |
| | sonication, finally add 5.489 mL | | |
| | of water (0.9% NaCl) with vortex | | |
| | to obtain the solution. | | |
| Compound B | 5% DMSO/40% PEG-400/55% | 10 | 4 |
| | water (0.9% NaCl) | | |
| | E.g., dissolve 39.92 mg of | | |
| | Compound A in 0.499 mL of | | |
| | DMSO with vortex and | | |
| | sonication, then add 3.992 mL of | | |
| | PEG-400 with vortex and | | |
| | sonication, finally add 5.489 mL | | |
| | of water (0.9% NaCl) with vortex | | |
| | to obtain the solution. | | |

### Sensitizing Agents

The first sensitizing agent (CFA+Collagen emulsion) was prepared by adding 1.5 mL Completed Freud's Adjuvant to a syringe with a 3-way stopcock and then adding dropwise into 1.5 mL bovine type II collagen (2 mg/mL) while mixing at 30,000 rpm for 2 minutes by a homogenizer. The emulsion was cooled in an ice water bath during the formulation. The final concentration of collagen was 1 mg/mL. The emulsion is prepared fresh immediately prior to injection.

The second sensitizing agent (IFA+Collagen emulsion) was prepared by adding 1.5 mL Incomplete Freud's Adjuvant to a syringe with a 3-way stopcock and then adding dropwise into 1.5 mL bovine type II collagen (2 mg/mL) while mixing at 30,000 rpm for 2 minutes by a homogenizer. The emulsion was cooled in an ice water bath during the formulation. The final concentration of collagen was 1 mg/mL. The emulsion is prepared fresh immediately prior to injection.

### Experimental Method B: Pharmacokinetics and Characterization of Compound A

### Rat Pharmacokinetics

### Rat Study #1

For the first rat pharmacokinetic study, the pharmacokinetics of Compound A were assessed in male Lewis rats. Compound A was prepared in two different formulations (1% methylcellulose and 20% propylene glycol/20% Vitamin E/60% water) according to Table 3A (see Tables 2A and 2D for formulations). The formulations were made fresh prior to use. Animals were divided into two groups based on the compound formulation (n=3/group). The Compound A solutions were administered to animals orally (PO) and animals were then monitored over a 24 h period.

TABLE 3A
Compound Formulations for Rat PK Study #1.

| | Dose Level | | Concentration |
|---|---|---|---|
| Compound | (mg/kg) | Preparation | (mg/mL) |
| Compound A | 10 | 1% methylcellulose | 1 |
| Compound A | 10 | 20% propylene | 1 |
| | | glycol/20% Vitamin | |
| | | E/60% water | |

Blood samples (~0.2 mL) were collected according to a schedule (0.083, 0.25, 0.5, 1, 2, 4, 8, and 24 h following the initial dosing) via the jugular vein by cannulation and EDTA-K₂ was used as anticoagulant. Within 30 minutes of collection, the samples were centrifuged at approximately 4000 times g-force for 5 minutes at 4° C. to obtain blood plasma. The plasma samples were immediately divided into two aliquots (~75 μL each), transferred to a cryogenic vial, and stored in a freezer at −75±15° C. prior to analysis.

The concentration of Compound A in plasma samples was determined by liquid chromatography-mass spectrometry (LC-MS/MS) using a Shimadzu HPLC (DGU-20A5R; LC-30AD; SIL-30AC; Rack changer II; CTO-31A; and CBM-20A) and an AB API 5500 LC/MS/MS instrument. Samples were analyzed on an Agilent Poroshell 120 EC-C18 4 μm (50×2.1 mm) column. Solution A in the mobile phase was 95% water (0.1% formic acid) and solution B was 95% acetonitrile in water (0.1% formic acid), injection volume was 3 μL, and flow rate was 0.6 mL/min.

To prepare samples for analysis, serial concentrations were prepared by diluting the stock solution of analyte with a 50% acetonitrile in water solution. 5 μL working solutions (5, 10, 20, 50, 100, 500, 1000, 5000, and 10000 ng/mL) were added to 50 μL of the blank Lewis rat plasma to achieve calibration standards of ~0.5 to 1000 ng/mL (0.5, 1, 2, 5, 10, 50, 100, 500, and 1000 ng/mL) in a total volume of 55 μL. Five quality control samples at 1 ng/mL, 2 ng/mL, 5 ng/mL, 50 ng/mL, and 800 ng/mL for plasma were prepared independently of those used for the calibration curves. These QC samples were prepared on the day of analysis in the same way as calibration standards.

55 μL of standards, 55 μL of QC samples and 55 μL of unknown samples (50 μL of unknown rat plasma with 5 μL of blank solution) were added to 200 μL of acetonitrile containing internal standard (IS; verapamil) mixture for precipitating protein respectively. The samples were then vortexed for 30 s. After centrifugation at 4° C., 3900 rpm for 15 min, the supernatant was diluted 3 times with water. 3 μL of diluted supernatant was injected into the LC/MS/MS system for quantitative analysis.

All data measured and recorded was analyzed using SPSS 16.0 software. Groups were compared using one-way ANOVA and significance was set at p<0.05.

### Rat Study #2

For the second rat pharmacokinetic study, the pharmacokinetics of Compound A were assessed in male SD rats. Compound A was prepared in a formulation of 5% DMSO, 40% PEG-400, and 55% Milli-Q water (see formulation in Table 2D) and delivered to animals orally (PO) at a dose of 5 mg/kg. Animals (n=3) were then monitored over a 24-hour period.

Blood samples (~0.2 mL) were collected according to a schedule (0.083, 0.25, 0.5, 1, 2, 4, 8, and 24 h following the initial dosing) by jugular vein by cannulation and EDTA-K₂ was used as anticoagulant. Within 30 minutes of collection, the samples were centrifuged at approximately 4000 times g-force for 5 minutes at 4° C. to obtain blood plasma. The plasma samples were immediately divided into two aliquots (~75 μL each), transferred to a cryogenic vial, and stored in a freezer at ~75±15° C. prior to analysis.

The concentration of Compound A in plasma samples was determined by liquid chromatography-mass spectrometry (LC-MS/MS) using a Shimadzu HPLC (DGU-405; LC-30AD XS CN; SIL-40AC XSCN; Plate Changer CN; CTO-40C CN; and CBM-40 CN) and an AB API 7500+LC/MS/MS instrument. Samples were analyzed on a Raptor Biphenyl 2.7 μm (50×2.1 mm) column. Solution A in the mobile phase was 95% water (0.1% formic acid) and solution B was 95% acetonitrile in water (0.1% formic acid), injection volume was 15 μL, and flow rate was 0.6 mL/min.

To prepare samples for analysis, serial concentrations were prepared by diluting the stock solution of analyte with a 50% acetonitrile in water solution. 5 μL working solutions (5, 10, 20, 50, 100, 500, 1000, 5000, and 10000 ng/mL) were added to 50 μL of the blank SD rat plasma to achieve calibration standards of ~0.5 to 1000 ng/mL (0.5, 1, 2, 5, 10, 50, 100, 500, and 1000 ng/mL) in a total volume of 55 μL. Five quality control samples at 1 ng/mL, 2 ng/mL, 5 ng/mL 50 ng/mL, and 800 ng/mL for plasma were prepared independently of those used for the calibration curves. These QC samples were prepared on the day of analysis in the same way as calibration standards.

55 μL of standards, 55 μL of QC samples and 55 μL of unknown samples (50 μL of unknown rat plasma with 5 μL of blank solution) were added to 200 μL of acetonitrile containing IS mixture (verapamil) for precipitating protein respectively. The samples were then vortexed for 30 s. After centrifugation at 4° C., 3900 rpm for 15 min, the supernatant was diluted 3 times with water. 15 μL of diluted supernatant was injected into the LC/MS/MS system for quantitative analysis.

All data measured and recorded was analyzed using SPSS 16.0 software. Groups were compared using one-way ANOVA and significance was set at p<0.05.

### Rat Study #3

For the third rat pharmacokinetic study, the pharmacokinetics of ABBV-744 were assessed in male SD rats for comparison to Compound A. ABBV-744 was prepared in a formulation of 5% DMSO, 40% PEG-400, and 55% Milli-Q water (see Table 2D for formulation) and delivered to animals orally (PO) at a dose of 5 mg/kg, or intravenously (IV) at a dose of 1 mg/kg. Animals in each group (n=3) were then monitored over a 24-hour period.

Blood samples (~0.2 mL) were collected according to a schedule (IV administration: 0.033, 0.083, 0.25, 0.5, 1, 2, 4, and 8 h following the initial dosing; PO administration. 0.083, 0.25, 0.5, 1, 2, 4, 8, and 24 h following the initial dosing) by jugular vein by cannulation except for the dosing vein (for IV group), and EDTA-K₂ was used as anticoagulant. Within 30 minutes of collection, the samples were centrifuged at approximately 4000 times g-force for 5 minutes at 4° C. to obtain blood plasma. The plasma samples were immediately divided into two aliquots (~75 μL each), transferred to a cryogenic vial, and stored in a freezer at ~75±15° C. prior to analysis.

The concentration of ABBV-744 in plasma samples was determined by liquid chromatography-mass spectrometry (LC-MS/MS) using a Shimadzu HPLC (DGU-405; LC-40D XS CN; SIL-40C XSCN; Plate Changer CN; CTO-40C CN; and CBM-40 CN) and an AB API 5500+LC/MS/MS instrument. Samples were analyzed on an Agilent Poroshell Bonus RP 2.7 μm (50×2.1 mm) column. Solution A in the mobile phase was 95% water (0.1% formic acid) and solution B was 95% acetonitrile in water (0.1% formic acid), injection volume was 6 μL, and flow rate was 0.6 mL/min.

To prepare samples for analysis, serial concentrations were prepared by diluting the stock solution of analyte with a 50% acetonitrile in water solution. 5 μL working solutions (5, 10, 20, 50, 100, 500, 1000, 5000, and 10000 ng/mL) were added to 50 μL of the blank SD rat plasma to achieve calibration standards of ~0.5 to 1000 ng/mL (0.5, 1, 2, 5, 10, 50, 100, 500, and 1000 ng/mL) in a total volume of 55 μL. Five quality control samples at 1 ng/mL, 2 ng/mL, 5 ng/mL, 50 ng/mL, and 800 ng/mL for plasma were prepared independently of those used for the calibration curves. These QC samples were prepared on the day of analysis in the same way as calibration standards.

55 μL of standards, 55 μL of OC samples and 55 μL of unknown samples (50 μL of unknown rat plasma with 5 μL of blank solution) were added to 200 μL of acetonitrile containing IS mixture (verapamil) for precipitating protein respectively. The samples were then vortexed for 30 s. After centrifugation at 4° C., 3900 rpm for 15 min, the supernatant was diluted 3 times with water. 6 μL of diluted supernatant was injected into the LC/MS/MS system for quantitative analysis.

All data measured and recorded was analyzed using SPSS 16.0 software. Groups were compared using one-way ANOVA and significance was set at p<0.05.

### Rat Study #4

For the fourth rat pharmacokinetic study, the pharmacokinetics of Compound A were assessed in male Lewis rats. Compound A was prepared in two different doses (0.2 mg/kg and 1.0 mg/kg) formulated in 20% propylene glycol/20% Vitamin E TPGS/60% water (see Table 1A for formulations). Animals were divided into two groups based on the compound formulation (n=3/group). The Compound A solutions were administered to animals orally (PO) and animals were then monitored over a 24 h period.

Blood samples (~0.2 mL) were collected according to a schedule (0.25, 0.5, 1, 2, 4, 6, 8, and 24 h following the initial dosing) by jugular vein by cannulation and EDTA-K₂ was used as anticoagulant. Within 30 minutes of collection, the samples were centrifuged at approximately 4000 times g-force for 5 minutes at 4° C. to obtain blood plasma. The plasma samples were immediately divided into two aliquots (~75 μL each), transferred to a cryogenic vial, and stored in a freezer at ~75±15° C. prior to analysis.

The concentration of Compound A in plasma samples was determined by liquid chromatography-mass spectrometry (LC-MS/MS) using a Shimadzu HPLC (DGU-20A5R; LC-30AD; SIL-30AC; Rack changer II; CTO-30A; and CBM-20A) and an AB API 5500+LC/MS/MS instrument. Samples were analyzed on an Agilent Poroshell 120 EC-C18 4 μm (50×2.1 mm) column. Solution A in the mobile phase was 95% water (0.1% formic acid) and solution B was 95% acetonitrile in water (0.1% formic acid), injection volume was 10 μL, and flow rate was 0.6 mL/min.

To prepare samples for analysis, serial concentrations were prepared by diluting the stock solution of analyte with a 50% acetonitrile in water solution. 5 μL working solutions (5, 10, 20, 50, 100, 500, 1000, 5000, and 10000 ng/mL) were added to 50 μL of the blank Lewis rat plasma to achieve calibration standards of ~0.5 to 1000 ng/mL (0.5, 1, 2, 5, 10, 50, 100, 500, and 1000 ng/mL) in a total volume of 55 μL. Five quality control samples at 1 ng/mL, 2 ng/mL, 5 ng/mL, 50 ng/mL, and 800 ng/mL for plasma were prepared independently of those used for the calibration curves. These QC samples were prepared on the day of analysis in the same way as calibration standards.

55 μL of standards, 55 μL of QC samples and 55 μL of unknown samples (50 μL of unknown rat plasma with 5 μL of blank solution) were added to 200 μL of acetonitrile containing IS mixture (verapamil) for precipitating protein respectively. The samples were then vortexed for 30 s. After centrifugation at 4° C., 3900 rpm for 15 min, the supernatant was diluted 3 times with water. 10 μL of diluted supernatant was injected into the LC/MS/MS system for quantitative analysis.

All data measured and recorded was analyzed using SPSS 16.0 software. Groups were compared using one-way ANOVA and significance was set at p<0.05.

### Dog Pharmacokinetics

The purpose of this study was to examine the pharmacokinetics of Compound A in beagle dogs. Four beagle dog studies were undertaken, one where Compound A was formulated in a propylene glycol based formulation (Dog PK study #1—PO and IV), a second where Compound A was formulated in a HPPCD based formulation (Dog PK study #2—IV), a third where Compound A was formulated in a 5% DMSO/15% PEG-400/80% (10% VE-TPGS) formulation (Dog PK study #3—PO), and a fourth where Compound A was formulated in % DMSO/15% PEG-400/80% (10% VE-TPGS) formulation (Dog PK study #4—PO).

Separate PK studies were also undertaken in rats—see Experimental Method B and Table 11E. Additional PK studies were undertaken as part of the CIA and UUO studies.

### Dog PK Study #1

For Dog PK study #1, two solutions of different concentrations of Compound A were prepared in 20% propylene glycol/20% Vitamin E TPGS/60% water as outlined in Table 2A in Experimental Method A. The solutions of Compound A in 20% propylene glycol/20% Vitamin E TPGS/60% water were prepared freshly prior to use. The Compound A solutions administered were then administered to animals either intravenously (IV) or orally (PO) and animals were then monitored over a 24 h period (Table 3B). Body weight was recorded prior to dosing, and detailed clinical observations were made prior to dosing and throughout the study, as needed.

TABLE 3B
Experimental treatment groups in Dog PK study #1

| | Number of | Compound A | | Administration |
|---|---|---|---|---|
| Group | animals | Concentration | Dose | Route |
| 1 | 3 | 0.5 mg/ml | 1 mg/kg | IV |
| 2 | 3 | 1.0 mg/ml | 5 mg/kg | PO |

Blood samples (~0.3 mL) were collected according to a schedule (shown below) by venipuncture of peripheral veins except for the dosing vein, and EDTA-K₂ was used as anticoagulant. Within 30 minutes of collection, the samples were centrifuged at approximately 2000 times g-force for 10 minutes at 4° C. to obtain blood plasma. The plasma samples were immediately divided into two aliquots (~75 μL each), transferred to a cryogenic vial, and stored in a freezer at −75±15° C. prior to analysis.

Blood samples for IV were collected at 0.083, 0.25, 0.5, 1, 2, 4, 8, 24 h and blood samples for PG were collected at 0.25, 0.5, 1, 2, 4, 8, and 24 h following the initial dosing (0 h).

The concentration of Compound A in the blood plasma was determined through liquid chromatography-mass spectrometry (LC-MS). The experiments were conducted on a Shimadzu HPLC (DGU-20A5R; LC-30AD; SIL-30AC; Rack Changer II; CTO-30A; and CBM-20A) and AB API 5500+LC/MS/MS instrument with an Agilent Poroshell EC-C18 4 μm (50×2.1 mm) column. A gradient of 95% water (0.1% formic acid)/95% acetonitrile (0.1% formic acid) was used as the mobile phase. The sample injection volume was 10 μL, and the flow rate was 0.6 mL/min. The gathered data were used for pharmacokinetic calculations using T½, C0, AUClast, AUCinf, MRTinf, Cl, Vss, and Number of Points for Regression as parameters.

The desired serial concentrations of working solutions were achieved by diluting stock solution of analyte with 50% acetonitrile in water solution. 5 μL of working solutions (5, 10, 20, 50, 100, 500, 1000, 5000, 10000 ng/mL) were added to 50 μL of the blank Beagle Dogs plasma to achieve calibration standards of 0.5-1000 ng/mL (0.5, 1, 2, 5, 10, 50, 100, 500, 1000 ng/mL) in a total volume of 55 μL. Five quality control samples at 1 ng/mL, 2 ng/mL, 5 ng/mL, 50 ng/mL and 800 ng/mL for plasma were prepared independently of those used for the calibration curves. These QC samples were prepared on the day of analysis in the same way as calibration standards.

55 μL standards, 55 μL QC samples and 55 μL unknown samples (50 μL plasma with 5 μL blank solution were added to 200 μL of acetonitrile containing IS mixture (verapamil) for precipitating protein respectively. Then the samples were vortexed for 30 s. After centrifugation at 4° C., 3900 rpm for 15 min, the supernatant was diluted 3 times with water. 10 μL of diluted supernatant was injected into the LC/MS/MS system for quantitative analysis.

### Dog PK Stud #2

For Dog PK study #2, a solution of Compound A was prepared in HPβCD (20% solution w/v)/DMSO (99/1) with a concentration of 0.5 mg/mL (Table 2B). The Compound A solution was administered to animals intravenously (IV), and animals were monitored over a 24 h period (Table 3C). Body weight was recorded prior to dosing, and detailed clinical observations were made prior to dosing and throughout the study, as needed.

TABLE 3C
Experimental treatment group in PK study 2

| | Number of | Compound A | | Administration |
|---|---|---|---|---|
| Group | animals | Concentration | Dose | Route |
| 1 | 3 | 0.5 mg/ml | 1 mg/kg | IV |

Blood samples (~0.3 mL) were collected according to a schedule (shown below) by venipuncture of peripheral veins except for the dosing vein, and EDTA-K₂ was used as anticoagulant. Within 30 minutes of collection, the samples were centrifuged at approximately 2000 times g-force for 10 minutes at 4° C. to obtain blood plasma. The plasma samples were immediately divided into two aliquots (~75 μL each), transferred to a cryogenic vial, and stored in a freezer at −75±15° C. prior to analysis.

Blood samples were collected at 0.083, 0.25, 0.5, 1, 2, 4, 8, and 24 h following the initial dosing (0 h).

The concentration of Compound A in the blood plasma was determined through liquid chromatography-mass spectrometry (LC-MS). The experiments were conducted on a Shimadzu HPLC (DGU-20A5R; LC-30AD; SIL-30AC; Rack Changer II; CTO-30A and CBM-20A) and AB API 5500 LC/MS/MS instrument with an Agilent Poroshell EC-C18 4 μm (50×2.1 mm) column. A gradient of 95% water (0.1% formic acid)/95% acetonitrile (0.1% formic acid) was used as the mobile phase. The sample injection volume was 4 μL, and the flow rate was 0.6 mL/min. The gathered data were used for pharmacokinetic calculations using T½, C0, AUClast, AUCinf, MRTinf, Cl, Vss, and Number of Points for Regression as parameters.

The desired serial concentrations of working solutions were achieved by diluting stock solution of analyte with 50% acetonitrile in water solution. 5 μL of working solutions (5, 10, 20, 50, 100, 500, 1000, 5000, 10000 ng/mL) were added to 50 μL of the blank Beagle Dogs plasma to achieve calibration standards of 0.5-1000 ng/mL (0.5, 1, 2, 5, 10, 50, 100, 500, 1000 ng/mL) in a total volume of 55 μL Five quality control samples at 1 ng/mL, 2 ng/mL, 5 ng/mL, 50 ng/mL and 800 ng/mL for plasma were prepared independently of those used for the calibration curves. These QC samples were prepared on the day of analysis in the same way as calibration standards.

55 μL standards, 55 μL QC samples and 55 μL unknown samples (50 μL plasma with 5 μL blank solution) were added to 200 μL of acetonitrile containing IS mixture (verapamil) for precipitating protein respectively. Then the samples were vortexed for 30 s. After centrifugation at 4° C., 3900 rpm for 15 min, the supernatant was diluted 3 times with water. 4 μL of diluted supernatant was injected into the LC/MS/MS system for quantitative analysis.

### Dog PK Study #3

For the third dog pharmacokinetic study, three solutions of different concentrations of Compound A were prepared in 5% DMSO/15% PEG-400/80% (10% VE-TPGS) as outlined in Table 2E. The Compound A solutions were administered to animals orally (PO) once daily over a five-day period. Body weight was recorded prior to dosing, and detailed clinical observations were made prior to dosing and throughout the study, as needed.

Animals were divided into Day 1 and Day 5 sample collection groups, with animals in the Day 1 collection group having blood samples taken prior to dosing and at 0.25, 0.5, 1, 2, 4, 8, and 24 hours after dosing on day 1. Animals in the Day 5 collection group having blood samples taken prior to dosing and at 0.25, 0.5, 1, 2, 4, 8, and 24 hours after dosing on day 5. Study groups are outlined in Table 3D.

TABLE 3D
Dog PK Study #3 Groups.

| | Group | Dose Level | Delivery | Collection |
|---|---|---|---|---|
| Group | Size | (mg/kg) | Route | Day |
| 1 | 2 | 5 | PO | Day 1 |
| 2 | 2 | 10 | PO | Day 1 |
| 3 | 2 | 20 | PO | Day 1 |
| 4 | 2 | 5 | PO | Day 5 |
| 5 | 2 | 10 | PO | Day 5 |
| 6 | 2 | 20 | PO | Dav 5 |

Blood samples (~0.3 mL) were collected on day according to the following schedule (0.25, 0.5, 1, 2, 4, 8, and 24 h) after day 5 administration of Compound A by venipuncture of peripheral veins except for the dosing vein, and EDTA-K₂ was used as anticoagulant. Within 30 minutes of collection, the samples were centrifuged at approximately 2000 times g-force for 10 minutes at 4° C. to obtain blood plasma. The plasma samples were immediately divided into two aliquots (~75 μL each), transferred to a cryogenic vial, and stored in a freezer at −75±15° C. prior to analysis.

The concentration of Compound A in the blood plasma was determined through LC-MS as previously described. Briefly, the experiments were conducted on a DMPK-LCMS-14-SMBA Triple Quad 6500 Plus instrument with an ACQUITY UPLC BEH C18 1.7 μm (50×2.1 mm) column. The matrix was male beagle dog plasma potassium (K₂) EDTA A gradient of 95% water (0.1% formic acid and 2 mM HCOONH4)/95% acetonitrile (0.1% formic acid and 2 mM HCOONH4) was used as the mobile phase. The sample flow rate was 0.6 mL/min.

20 μL of standards, 20 μL of QC samples, 20 μL of unknown samples were added to 200 μL of acetonitrile containing IS mixture (labetalol, tolbutamine, verapamil, dexamethasone, glyburide, and celecoxib, each at a concentration of 100 ng/mL) for precipitating protein, respectively. The samples were then vortex-mixed for 10 minutes at 800 rpm and centrifuged for 15 minutes at 3220×g at 4° C. 50 μL aliquots of supernatant were transferred to a clean 96-well plate and centrifuged for 5 minutes at 3220×g at 4° C. Samples were then injected into the LC/MS/MS system for quantitative analysis.

The gathered data were used for pharmacokinetic calculations using T½, C0, AUClast, AUCinf, MRTinf, Cl, Vss, and Number of Points for Regression as parameters.

The cell composition (hematology) of blood samples was analyzed at day 5. Analyses identified the total number of white blood cells, neutrophils, lymphocytes, monocytes, eosinophils, basophils, and platelets in collected samples.

### Dog PK Study #4

For Dog PK Study #4, three different concentrations of Compound A (1 mg/kg, 3 mg/kg, and 10 mg/kg) were prepared in 5% DMSO/15% PEG-400/80% (10% VE-TPGS) as outlined in Table 2E. The different doses of compound A were orally administered to animals (n=2/group, one male and one female animal) daily for 14 days.

Blood samples were collected at 0.5, 1, 2, 4, 8, and 24 hours after administration of Compound A on Day 14, and EDTA-K₂ was used as an anticoagulant. Within 30 minutes of collection, the samples were centrifuged at approximately 2000 xg for 10 minutes at 4° C. to obtain blood plasma. The samples were immediately divided into aliquots, transferred to cryogenic vials, and stored in a freezer at −75±15° C. prior to analysis.

The concentration of Compound A in the blood plasma was determined through liquid chromatography-mass spectrometry (LC-MS). The experiments were conducted on a Shimadzu UPLC and AB API 6500+LC/MS/MS with a Water ACQUITIY UPLC HSS T3 1.8 μm (50×2.1 mm) column. A gradient of 0.1% formic acid in water/0.1% formic acid in acetonitrile was used as the mobile phase. The sample injection volume was 1 μL. The flow rate was 0.6 mL/min.

The desired serial concentrations of working solutions were achieved by diluting 30 μL of sample (control standard, QC standard, test samples) with 30 μL of a 50% acetonitrile in water solution (for double blanks) or 30 μL of the IS mixture (Compound B 80 ng/mL in 50% acetonitrile). 240 μL of 100% acetonitrile was then added and samples were vortexed for 5 min and centrifuged at 3200×g at 4° C. for 10 min. 100 μL of the supernatant was transferred and then diluted with 50% acetonitrile. Samples were then vortexed for about 5 min before injection into the LC/MS/MS system for quantitative analysis.

### In Vitro Characterization of Compound A Selectivity

To analyze the selectivity of Compound A for Th17 cytokines in vitro, human PBMCs (STEMCELL Technologies) were thawed in RPMI-1640 medium supplemented with 10% FBS in a round bottom 96-well tissue culture plate. Human PBMCs used to analyze inhibition of CXCL10 were collected from 8 donors, PBMCs used to analyze inhibition of IL-17A were collected from 7 donors, and PBMCs used to analyze inhibition of IL-22 were collected from 6 donors. Compounds were serially diluted in 10 mM DMSO and further diluted in assay media to 100× the required final concentration, of which 2 μL was added to the specified well. Compound concentrations ranged from 100 nM to 0.015 nM and the final DMSO concentration per well was 0.1%. For analysis of the selectivity of Compound A for IL-17A and IL-22, cultured human PBMCs (2×10⁵ cells/well) were activated with anti-CD2, anti-CD3, and anti-CD28 antibodies (Miltenyi Biotec) at a ratio of 1:2 in the presence of Compound A for 72 hours (total volume per well was 200 μL). The cell culture supernatant was collected and levels of IL-17A and IL-22 in the supernatant were assessed by ELISA (Invitrogen).

To analyze the selectivity of Compound A for CXCL10, human PBMCs were similarly thawed, and compounds were similarly prepared (final compound concentration ranged from 30,000 nM to 0.005 nM, with a final DMSO concentration per well of 0.3%). PBMCs were seeded into 96-well plates at a concentration of 2×105 cells/well (volume of 190 μL) and treated with various concentrations of Compound A for 1 hour. The PBMCs were then stimulated with IFN-γ (15 ng/mL final concentration in a volume of 10 μL), and supernatants were collected 24 hours after the exposure to IFN-γ. Levels of CXCL10 in the supernatant were then analyzed by ELISA (Invitrogen).

### Experimental Method C: Psoriasis in Animals Treated with BETi Formulations and Vehicle

Imiquimod-induced psoriasis is a model of psoriasis in mice that induces psoriasis-like dermatitis. The pathology closes resembles human plaque-type psoriasis and is a valuable tool for testing new therapies against psoriasis. The imiquimod-induced psoriasis model was used to assess the efficacy of intraperitoneal delivery of Compound A and Compound B (50 μL) at three different concentrations (1 mg/kg, 3 mg/kg, and 10 mg/kg). Compound A was formulated in a 5% DMSO/40% PEG-400/55% (0.9% NaCl w/v water) solution (Table 2F). Experimental groups for the study are outlined in Table 4. Prior to study initiation, the backs of all animals in the study were depilated to allow for induction of psoriasis and monitoring of disease progression.

TABLE 4
Experimental Groups for Psoriasis Study.

| Group | Treatment | Dose Level | | IMI | N | Route |
|---|---|---|---|---|---|---|
| 1 | Vehicle | — | | No* | 8 | I.P. |
| 2 | Vehicle | — | | Yes | 8 | I.P. |
| 3 | Compound A | 1 | mg/kg | Yes | 8 | I.P. |
| 4 | Compound A | 3 | mg/kg | Yes | 8 | I.P. |
| 5 | Compound A | 10 | mg/kg | Yes | 8 | I.P. |
| 6 | Compound B | 1 | mg/kg | Yes | 8 | I.P. |
| 7 | Compound B | 3 | mg/kg | Yes | 8 | I.P. |
| 8 | Compound B | 10 | mg/kg | Yes | 8 | I.P. |
| 9 | Apremilast | 3 | mg/kg | Yes | 8 | I.P. |
| 10 | Deucravacitinib | 3 | mg/kg | Yes | 8 | I.P. |

| | | | | | |
|---|---|---|---|---|---|
| 11 | Clobetasol | 0.05% | Yes | 8 | Topical |

*Animals in group 1 received topical administration of Excipial ® hydrocream instead of IMI.

Once assigned to experimental groups, on Day 1 and continuing through Day 14, topical imiquimod (IMI; Aldara™ containing 5% Imiquimod) was administered to the dorsal depilated animals, and animals in group 1 received topical administration of Excipial® hydrocream (65 mg). Starting on Day 8 and continuing through Day 14, the study drug (i.e., Compound A) or other designated treatment (e.g., Deucravacitinib, Apremilast, or placebo) was administered through intraperitoneal (IP) injection once daily. Clobetasol was applied topically for animals in group 11.

Body weights were recorded three times a week from Day 1 to Day 8, and daily from Day 8 until the study end at Day 15. At termination on D15 the spleen weight ratio was determined. Disease severity was assessed using the psoriasis area severity index (PASI) daily from Day 8 until study completion on Day 15. The PASI score was calculated based on three parameters (presence of erythema, induration of the skin, and peeling of the skin at the area of induction) on a scale of 0 to 4 (0=no more or absence, 1=low, 2=moderate, 3=high, 4=severe). The PASI score for each animal was calculated as the sum of the score of each parameter measured. The score of the use of enrichment (SUE) was also assessed daily from Day 8 to Day 15. At study completion on Day 15, animal behavior was evaluated using an open field to assess distance travelled, number scratching, and licking and rearing performed in 10 minutes. Upon study termination animals were sacrificed, the spleen was excised and weighed, and blood samples were collected. Serum was isolated from the blood sample for cytokine and other biomarker analysis. IL-1β, IL-6, IL-17, and TNF-α assayed using simple plex technique on an ELLA automated immunoassay system. IL-22 and IL-23 were assayed by ELISA.

### Experimental Method D: Collagen-Induced Arthritis in Animals Treated with BETi Formulations and Vehicle

Collagen-induced arthritis (CIA) is a complex model of autoimmune-mediated arthritis. CIA is induced in rats by immunization with type I1 collagen and develops joint arthritis pathology that is similar to rheumatoid arthritis. Accordingly, CIA has been used extensively as a valuable model for studying rheumatoid arthritis.

The collagen-induced arthritis model in rats was used to assess the efficacy of oral delivery of Compound A at three concentrations (0.1 mg/mL, 0.3 mg/mL, and 1 mg/mL). Experimental groups for the study are outlined in Table 5A. Prior to being randomly placed in treatment groups, animals were weighed, and paw volume was measured. Once randomly assigned to treatment groups, on Day 0, animals in groups 2-9 were immunized by subcutaneous injection at the base of the tail. The immunization comprised 100 μL of the CFA+Collagen emulsion (prepared according to the method in Experimental Method A—Sensitizing Agents). Animals in group one received no immunization. Seven days after the first injection, animals in groups 2-9 received a booster immunization comprising 200 μL of the IFA+Collagen emulsion (prepared according to the method in Experimental Method A—Sensitizing Agents). Animals in group one did not receive a booster immunization.

TABLE 5A
Experimental Groups.

| | | | | | | | | Paw volume, |
|---|---|---|---|---|---|---|---|---|
| | | | | | | | | Clinical |
| | | | | | | | | score, and |
| | | Dose Level | | | | Dosing volume | | blood |
| Group | Treatment | (mg/kg) | CIA | N | Frequency | (ml/kg) | Route | collection |
| 1 | Vehicle | — | No | 8 | QD*21 | 10 | P.O. | Day 6, 12, 13, |
| 2 | Vehicle | — | Yes | 8 | QD*21 | 10 | P.O. | 14, 15, 18, 19, |
| 3 | Dexamethasone | 0.1 | Yes | 8 | QD*21 | 5 | P.O. | and 21 (8 |
| 4 | GSK620 | 10 | Yes | 8 | QD*21 | 5 | P.O. | times); Plasma |
| 5 | Compound A | 1 | Yes | 8 | QD*21 | 10 | P.O. | & serum on |
| 6 | Compound A | 3 | Yes | 8 | QD*21 | 10 | P.O. | day 21 |
| 7 | Compound A | 10 | Yes | 8 | QD*21 | 10 | P.O. | |
| 8 | Compound A | 3 | Yes | 3 | QD*21 | 10 | P.O. | Plasma |
| | | | | | | | | collection on |
| | | | | | | | | day 0 |
| 9 | Compound A | 3 | Yes | 3 | QD*21 | 10 | P.O. | Plasma |
| | | | | | | | | collection on |
| | | | | | | | | day 20 |

The structure of GSK620 is

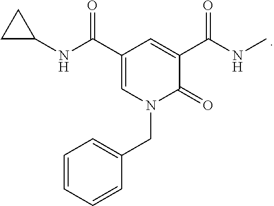 [SMILES: CNC(=O)c1cc(C(=O)NC2CC2)cn(Cc2ccccc2)c1=O]

Starting on Day 0, animals in group 1 and 2 were administered daily doses of the vehicle per os (P.O.) for 21 days. Starting on Day 0, animals in groups 3 and 4 were administered daily doses of dexamethasone and GSK620, respectively, P.O. for 21 days. Starting on Day 0, animals in groups 4-9 were administered daily doses of Compound A according to the dosages outline in Table 5A for 21 days.

Body weight of all animals was recorded daily for the 21-day period. Blind clinical scores on all four limbs were conducted for all animals in the study. Assessments were recording according to the clinical scoring system summarized in Table 5B. Animals were assessed prior to receiving their daily dose of the compound or control, and scores for each limb were combined to a total daily score for each animal. Clinical score assessments were performed on Day 0, 6, 12, 13, 14, 15, 18, 19, and 21 (for a total of 9 daily scores).

TABLE 5B
Clinical scoring rubric

| Score | Condition |
|---|---|
| 0 | Normal |
| 1 | Mild, but definite redness and swelling of the ankle |
| | or wrist, or apparent redness and swelling limited to |
| | individual digits (regardless of the number of digits |
| | affected) |
| 2 | Moderate redness and swelling or ankle or wrist |
| 3 | Severe redness and swelling of the entire paw, |
| | including digits |
| 4 | Maximally inflamed limb with involvement of multiple |
| | joints |

Paw volume was recorded for each animal on Day 0, 6, 12, 13, 14, 15, 18, 19, and 21 (for a total of 9 measurements). To measure paw volume, animals were gently held with one hand while the hind paw was placed into a plethysmometer with the other hand. Lipid displacement by the paw was recorded and used to represent paw volume.

At the conclusion of the study (end of the 21-day period), animals were euthanized. Euthanasia occurred four hours after animals received their first dose on the morning of Day 21. Hind limbs were collected following euthanasia, with the left limb being used for H&E and IHC staining. IHC markers for staining were selected after evaluating clinical and histopathological scores for animals. After the skin was removed, the right hind limb was snap frozen in liquid nitrogen and RNA and protein analysis was performed.

Blood samples were collected from animals in groups 1-7 via cardiac puncture following euthanasia on Day 21. Blood from cardiac puncture was placed in a 1.5 mL centrifuge tube without an anticoagulant and centrifuged at 8000 rcf for 15 minutes. Serum was divided into two separate tubes and stored at −75±15° C. One tube was used to analyze IgG1, and the other tube was reserved for additional analyses.

Blood samples were collected from animals in group 8 on Day 0, and blood samples were collected from group 9 on Day 20. Blood samples from group 8 and group 9 were collected at 0.25, 0.5, 1, 2, 4, 8, 10, 12, 13, and 24-hours following morning administration of their first dose of the compound. The second dose of the compound was administered after the 8-hour collection timepoint. All blood samples were collected at the eye socket and put into tubes containing EDTA-K2 as an anticoagulant and centrifuged at 4° C. at 4,000 g for 5 minutes. Plasma was transferred to a separate tube and stored at −75±15° C. for PK analysis. Pharmacokinetic analyses for Compound A in Lewis rats were performed as described in Experimental Method B.

H&E-stained slides of designated tissues from animals were transferred to an independent pathologist for microscopic evaluation (SPFBio). Samples were assigned the histologic grades based on the severity of change from tissue where CIA was not induced. Histology grades assigned were as follows: grade 1 (minimal change), grade 2 (mild change), grade 3 (moderate change), grade 4 (marked change), and grade 5 (severe change). A grade may be assigned for each indicator of disease severity: inflammation, mixed cell; granulation tissue; increased bone, periosteum; increased eroded surface, bone and erosion/ulcer, cartilaginous. A combined description may be provided if animals within a treated group exhibit different adjoining levels of change. By way of example, the following observations for each indicator were made respectively in GSK620 treated rats with CIA and in vehicle treated rats with CIA with more marked changes being described for the latter group. Mild to moderate mixed cell inflammation was characterized by widespread accumulation of neutrophils and macrophages with lesser numbers of lymphocytes within one or more joint spaces, synovium, and periarticular soft tissues. Moderate inflammation was also associated with increased edema fluid, fibrin, and cellular debris. Marked mixed cell inflammation was characterized by dense, widespread accumulations of neutrophils and macrophages with lesser number of lymphocytes within one or more joint spaces, synovium and periarticular soft tissues. Marked inflammation was also associated with abundant edema fluid, fibrin, and cellular debris.

Mild to moderate granulation tissue (pannus) was associated with inflammation and characterized by widespread fibrovascular connective tissue that expanded the periarticular soft tissues of one or more joints, extended into joint spaces, was contiguous with areas of erosion/ulcer in articular cartilage and variably extended along the diaphysis. Marked granulation tissue (pannus) was associated with inflammation and was characterized by widespread fibrovascular connective tissue that expanded/replaced the periarticular soft tissues of one or more joints, extended into/expanded joint spaces, was contiguous with areas of erosion/ulcer in articular cartilage and/or increased eroded surface of bone, and variably extended along the diaphysis.

Minimal to moderate increased eroded surface of bone (bone resorption) affected both trabecular bone, and cortical bone was characterized by irregular, scalloped bone surfaces, increased numbers of osteoclasts, and/or disruption of lamellae involving one bone. Instances of minimal increased eroded surface were not associated with fragments of bone contiguous and/or surrounded by granulation tissue. Moderate to marked increased eroded surface of bone (bone resorption) affected both trabecular bone and cortical bone and was characterized by irregular, scalloped bone surfaces, increased numbers of osteoclasts, and/or disruption of lamellae involving one or more bones. In some areas, increased eroded surfaces were contiguous with areas of erosion/ulcer in the articular cartilages. Instance of marked increased eroded surface were often associated with fragments of bone contiguous with and/or surrounded by granulation tissue.

Mild to moderate erosion/ulcer in the articular cartilages was characterized by local area of incomplete (erosion) loss of the articular cartilage at one or more bones. Moderate to marked erosion/ulcer in articular cartilages was characterized by multifocal areas of incomplete (erosion) or complete (ulcer) loss of articular cartilage at one or more bones. This observation was typically most pronounced at joint margins (transition zone). The ulceration was often associated with inflammation and granulation tissue extending through the defect to the subchondral bone.

Mild to moderate increased periosteal bone (periosteal bone formation) was characterized by local areas of increased bone matrix and numerous prominent osteoblasts lining trabecular surfaces along the metaphysis of one or more bones. In some areas, increased periosteal bone was contiguous with increased eroded surface and at articular cartilage. Marked increased periosteal bone (periosteal bone formation) was characterized by extensive areas of increased bone matrix and numerous prominent osteoblasts lining trabecular surfaces along the metaphysis and/or diaphysis of one or more bones. In some areas, increased periosteal bone was contiguous with increased eroded surface and/or erosion/ulcer at articular cartilage.

All data measured and recorded was analyzed using SPSS 16.0 software. Groups were compared using one-way ANOVA and significance was set at p<0.05.

### Experimental Method E: Unilateral Urethral Obstruction in Animals Treated with BETi Formulations and Vehicle

Unilateral Urethral Obstruction (UUO) is a complex model of renal fibrosis (RF) disease. RF is a common outcome in progressive chronic kidney diseases, characterized by excessive formation of internal scar tissue. Surgical obstruction of urine flow can be used to trigger this disease in rodents. The UUO model allows for evaluation of potential drugs as treatment for RF.

The UUO model in rats was used to assess the efficacy of oral delivery of Compound A at a 1 mg/mL concentration. Experimental groups for the study are outlined in Table 6. Prior to be randomly placed in treatment groups, animals were weighed. Once randomly assigned to treatment groups, on Day 0, animals in groups 2-4 were subjected to UUO surgery. Briefly, animals were anesthetized using a Zoletil/Xylazine mixture (20:1, v:v) for a final dose of 25 mg/kg. The operation area was disinfected with 75% alcohol before shaving. Rats in UUO group were exposed left ureter through a small incision on the left side of the abdomen and ligated with 3-0 silk at two locations about 0.5 cm below the renal hilum. The ureters of sham rats in Group 1 were manipulated but not ligated.

TABLE 6
Experimental Groups.

| | | | | | | Clinical |
|---|---|---|---|---|---|---|
| | | | | | | observation, |
| | | | | | | body weight |
| | | | | | | measurement, |
| | | | | | | clinical |
| | | Dose | | | Dosing | nephropathy |
| | | Level | | | volume | score, blood and |
| Group | Treatment | (mg/kg) | UUO | N | (ml/kg) | tissue collection |
| 1 | Sham | — | No | 8 | 10 | Body Weight |
| | | | | | | measured Days- |
| | | | | | | 1-14; Clinical |
| 2 | Vehicle | — | Yes | 8 | 10 | observation |
| | | | | | | assessed |
| | | | | | | Days 0-14; |
| | | | | | | Clinical |
| | | | | | | Score determined |
| | | | | | | and |
| 3 | Compound | 10 | Yes | 8 | 10 | Blood and Tissue |
| | A | | | | | collected on |
| | | | | | | Day 14 |

Starting on Day 0, animals in group 2 were administered daily doses of the vehicle for 14 days. Starting on Day 0, animals in group 3 were administered daily doses of Compound A (10 mg/kg) for 14 days.

Body weight of all animals was recorded daily for the 14-day period. Clinical signs for all animals were monitored and recorded daily. Blind clinical nephropathy scores were conducted for all animals in the study. Assessments were recording according to the clinical scoring system summarized in Table 7. Glomerulosclerosis, interstitial nephritis, collagen fiber deposition, nephropathy were each assessed for all experimental rats, and a total clinical score was determined for each rat. The scoring system represented the degree or percentage of lesions in the whole kidney. Clinical score assessments were performed on Day 14.

TABLE 7
Clinical scoring rubric

| Score | Condition |
|---|---|
| 0 | Normal, <15% lesions in the whole kidney |
| 1 | Mild, 15%−<30% lesions in the whole kidney |
| 2 | Moderate, 30%−<50% lesions in the whole kidney |
| 3 | Severe, 50%−<80% lesions in the whole kidney |
| 4 | Very Severe, ≥80% lesions in the whole kidney |

Serum for blood biochemistry analysis was collected into centrifuge tubes without coagulant four hours after final dosing. Blood plasma samples from group 3 was collected into tubes with K₂EDTA four hours after final dosing. At the conclusion of the study (end of the 14-day period), animals were euthanized. Kidney tissue was collected four hours after final dosing. Ligated side kidney was divided into three copies ⅓ fix into 10% formalin for H&E analysis, ⅓ into RNA for PCR analysis of tissue biomarkers Col1a1, TGF-b1, MCP-1, IL-1b, IL-6, IL-17, TNF-a, and Timp1, and ⅓ for hydroxyproline assay analyzed by ELISA. RNA was extracted from tissue using the RNeasy mini kit (Qiagen). RNA concentration was measured using a NanoDrop One Spectrophotometer (ThermoFisher) and converted to cDNA using a High Capacity RNA-to-cDNA kit (Invitrogen). RT-PCR was performed to measure levels of mRNA of different targets using TaqMan® Gene Expression Master Mix and TaqMan® probe/primer sets on a QuantStudio 6 Real-Time PCR System (ThermoFisher). Fold change in target gene expression was determined using the AACt method.

Concentration of Compound A in plasma samples was determined by liquid chromatography-mass spectrometry (LC-MS/MS) using a Shimadzu HPLC (DGU-20A5R; LC-30AD; SIL-30AC; and Rack changer II) and an AB AP140000 LC/MS/MS instrument. Samples were analyzed on a Raptor Biphenyl 2.7 μm 50×2.1 mm column. Solution A in the mobile phase was 95% water (0.1% formic acid) and solution B was 95% acetonitrile in water (0.1% formic acid), injection volume was 20 μL, and flow rate was 0.5 mL/min.

To prepare samples for analysis, serial concentrations were prepared by diluting the stock solution of analyte with a 50% acetonitrile in water solution. 5 μL working solutions (5, 10, 20, 50, 100, 500, 1000, 5000, and 10000 ng/mL) were added to 50 μL of the blank SD rat plasma to achieve calibration standards of ~0.5 to 1000 ng/mL (0.5, 1, 2, 5, 10, 50, 100, 500, and 1000 ng/mL) in a total volume of 55 μL. Four quality control samples at 1 ng/mL, 2 ng/mL, 50 ng/mL, and 800 ng/mL for plasma were prepared independently of those used for the calibration curves. These QC samples were prepared on the day of analysis in the same way as calibration standards.

55 μL of standards, 55 μL of QC samples and 55 μL of unknown samples (50 μL of unknown rat plasma with 5 μL of blank solution) were added to 200 μL of acetonitrile containing IS mixture for precipitating protein respectively. The samples were then vortexed for 30 s. After centrifugation at 4° C., 4000 rpm for 15 min, the supernatant was diluted 3 times with water. 20 μL of diluted supernatant was injected into the LC/MS/MS system for quantitative analysis.

All data measured and recorded was analyzed using SPSS 16.0 software. Groups were compared using one-way ANOVA and significance was set at p<0.05.

### Experimental Method F: Synthesis of Compounds

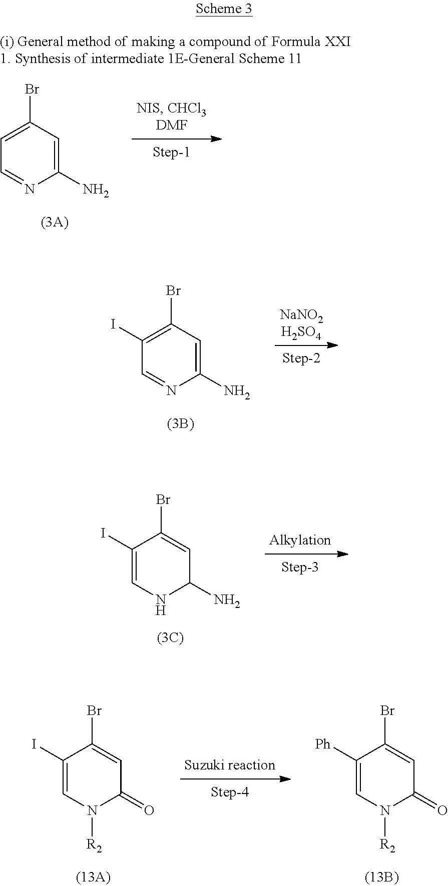

General Scheme 11 illustrates a general route for the preparation of general intermediate (13B). From (3A), iodination, pyridone formation, alkylation and Suzuki reaction can result in (13B).

### 2. Synthesis of Intermediate 2G—General Scheme 12

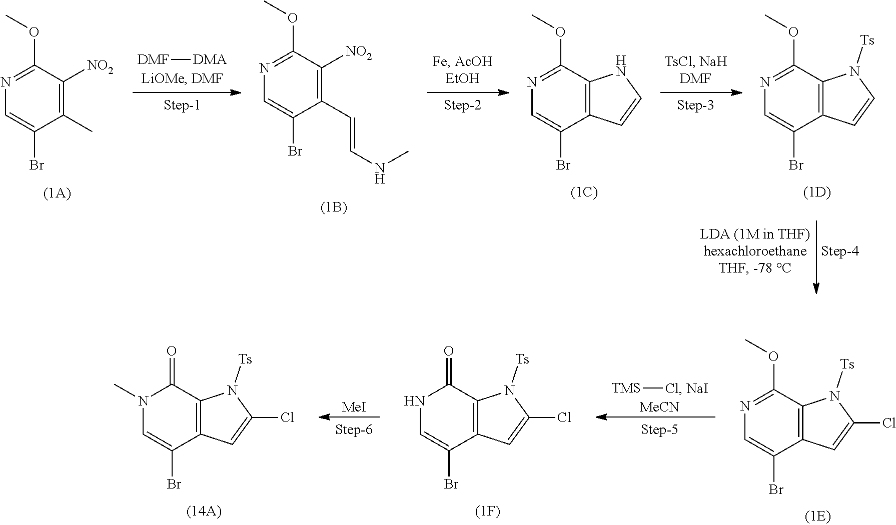

General Scheme 12 illustrates the route for the preparation of intermediate (14A) of the disclosure.

### 3. Synthesis of Compounds of Formula XXI—General Scheme 13

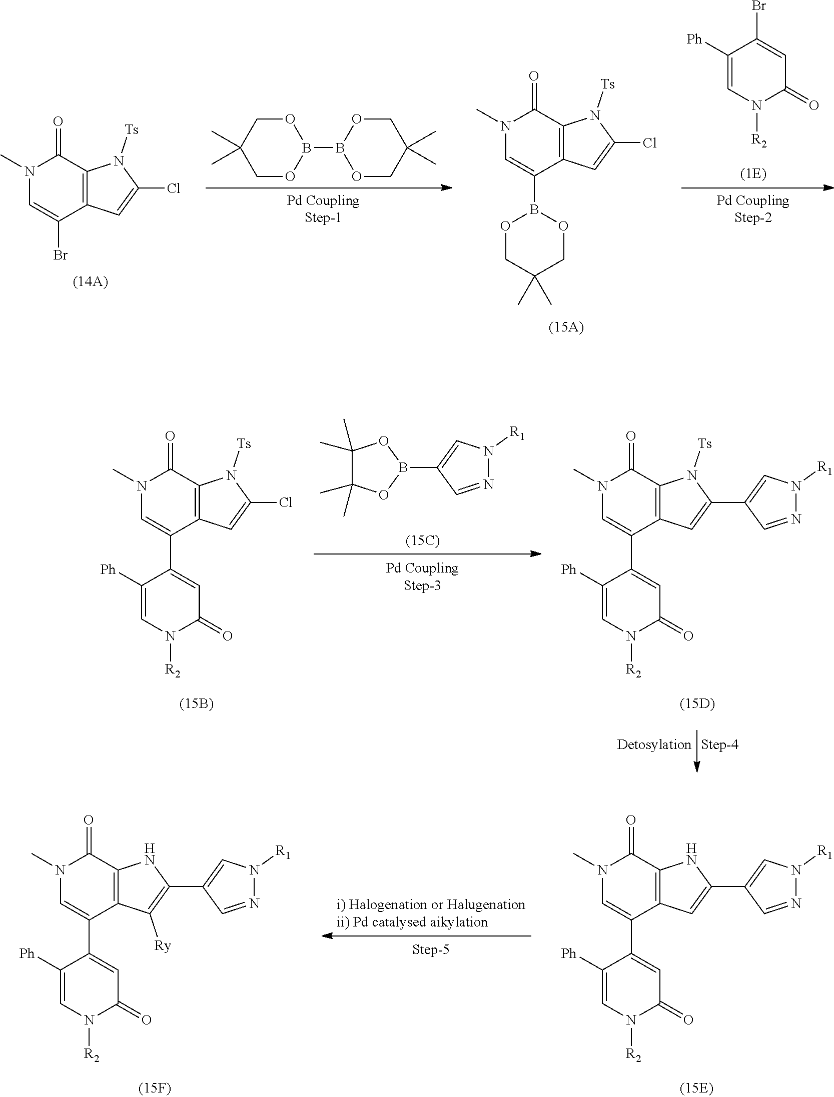

General Scheme 13 illustrates a general route for the preparation of compounds of the disclosure. From (14A) Pd catalysed boronate formation, Pd catalysed Suzuki coupling and Pd catalysed Suzuki coupling followed by detosylation gave compounds of the disclosure such as (15E). Subsequent halogenation or halogenation and Pd catalysed alkylation gave compounds of the disclosure such as (15F).

### (ii) General Method of Making a Compound of Formula XXIA

### 1. Synthesis of Intermediate 3E—General Scheme 14

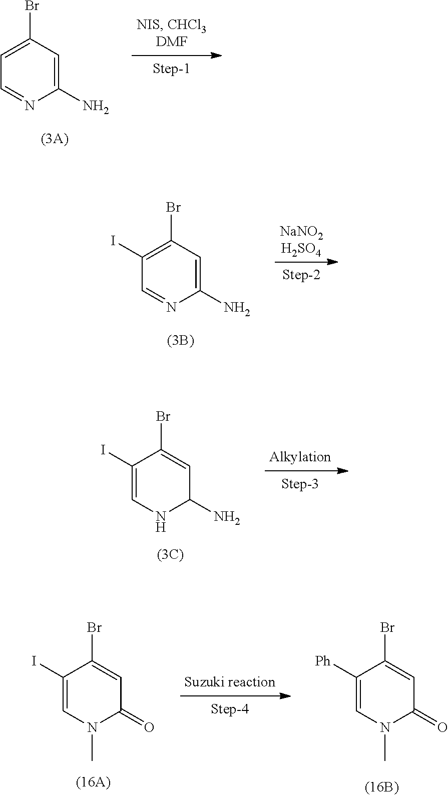

The above reaction scheme illustrates a preparation of intermediate (16B) of the disclosure. From (3A), iodination, pyridone formation, alkylation and Suzuki reaction can result in (16B).

### 2. Synthesis of Compounds of Formula XXIA—General Scheme 15

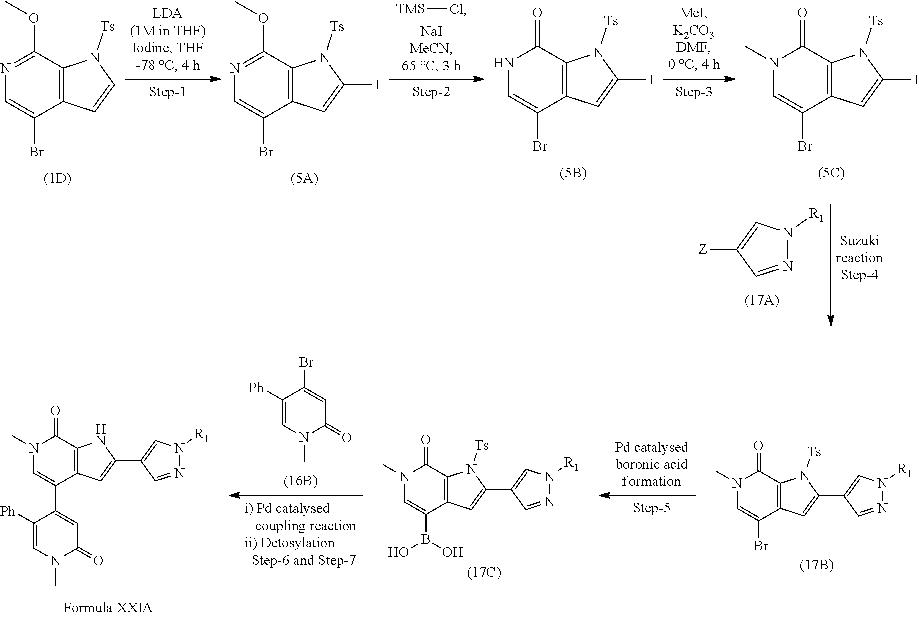

The above reaction scheme illustrates a general route for the preparation of compounds of Formula XXIA of the present disclosure. From (5C) Suzuki coupling followed by boronic acid formation, Pd coupling and detosylation can result in compounds of the disclosure (Formula XXIA).

### Example 117: Pharmacokinetics and Characterization of BET Inhibitors

Eight studies were undertaken to assess the pharmacokinetics of Compound A: four in rats (rat PK study 1, 2, 3, and 4), and four in dogs (dog PK study 1, 2, 3, and 4). Preparation and evaluation of Compound A was undertaken according to the protocols set out in Experimental Method B. Additional PK studies were undertaken as part of the CIA and UUO Studies.

### Rat Pharmacokinetic Studies

### Rat PK Study #1

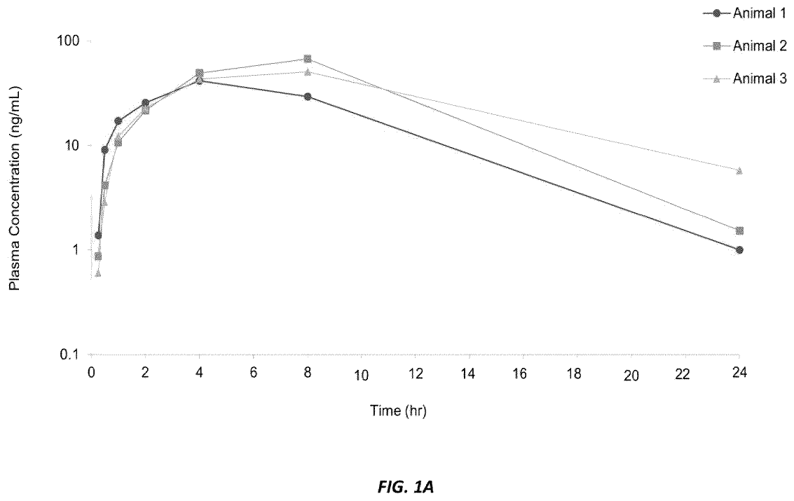

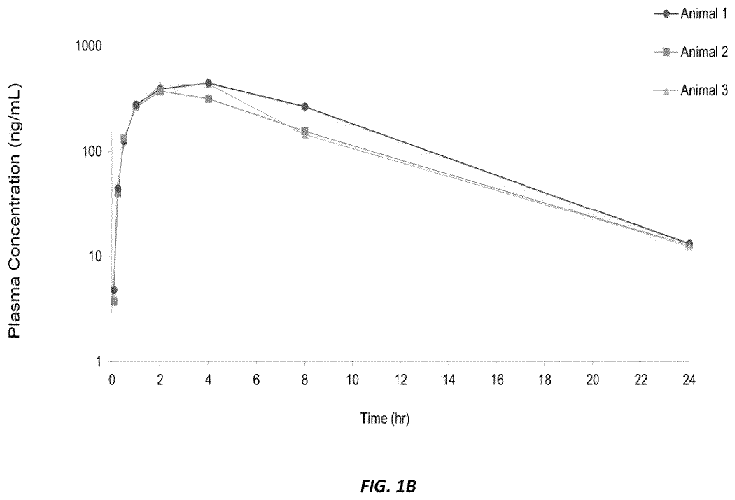

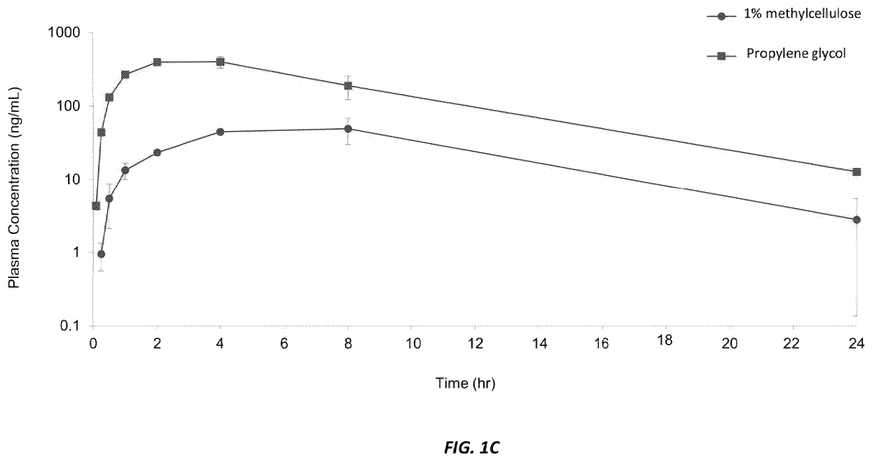

The first rat pharmacokinetic study evaluated two different formulations of Compound A: a propylene glycol-based formulation, and a methylcellulose-based formulation. FIGS. 1A and 1B illustrate the plasma concentration of Compound A over time for the methylcellulose-based formulation (FIG. 1A, Table 8A) and the propylene glycol-based formulation (FIG. 1B, Table 8B). Both formulations demonstrated a sharp rise in plasma concentration shortly after compound administration, although the propylene glycol-based formulation had plasma concentrations that were approximately 10-fold higher than the methylcellulose-based formulation, despite the same dose of the compound being administered to animals (FIG. 1C). Tables 8C and 8D show a summary of pharmacokinetic parameters for the methylcellulose-based and propylene glycol-based formulations, respectively.

TABLE 8A
Compound A Plasma Concentration in Methylcellulose-based
Formulation.

| Time | Animal 1 | Animal 2 | Animal 3 | Mean | SD | CV |
|---|---|---|---|---|---|---|
| (h) | (ng/ml) | (ng/ml) | (ng/ml) | (ng/ml) | (ng/ml) | (%) |
| 0.083 | 0.00 | 0.00 | 0.00 | BLOQ | — | — |
| 0.25 | 1.37 | 0.868 | 0.603 | 0.95 | 0.39 | 41.1 |
| 0.5 | 9.07 | 4.15 | 2.89 | 5.37 | 3.27 | 60.8 |
| 1 | 17.1 | 10.7 | 12.2 | 13.3 | 3.3 | 25.1 |
| 2 | 25.5 | 21.6 | 22.7 | 23.3 | 2.0 | 8.64 |
| 4 | 41.5 | 49.2 | 43.1 | 44.6 | 4.1 | 9.11 |
| 8 | 29.3 | 67.3 | 50.6 | 49.1 | 19.0 | 38.8 |
| 24 | 0.996 | 1.52 | 5.80 | 2.77 | 2.64 | 95.1 |

TABLE 8B
Compound A Plasma Concentration in propylene
glycol/Vitamin E TPGS/Water-based Formulation.

| Time | Animal 1 | Animal 2 | Animal 3 | Mean | SD | CV |
|---|---|---|---|---|---|---|
| (h) | (ng/ml) | (ng/ml) | (ng/ml) | (ng/ml) | (ng/ml) | (%) |
| 0.083 | 4.79 | 3.72 | 4.36 | 4.29 | 0.54 | 12.6 |
| 0.25 | 44.5 | 39.8 | 47.2 | 43.8 | 3.7 | 8.54 |
| 0.5 | 125 | 134 | 134 | 131 | 5 | 3.97 |
| 1 | 277 | 264 | 263 | 268 | 8 | 2.91 |
| 2 | 390 | 375 | 424 | 396 | 25 | 6.33 |
| 4 | 445 | 316 | 437 | 399 | 72 | 18.1 |
| 8 | 266 | 156 | 145 | 189 | 67 | 35.4 |
| 24 | 13.1 | 12.6 | 12.5 | 12.7 | 0.3 | 2.52 |

TABLE 8C
Compound A Pharmacokinetics in Methylcellulose-based Formulation.

| | | Animal | Animal | Animal | | | CV |
|---|---|---|---|---|---|---|---|
| Time (h) | Unit | 1 | 2 | 3 | Mean | SD | (%) |
| T_{1/2} | h | — | — | — | — | — | — |
| Tₘₐₓ | h | 4.00 | 8.00 | 8.00 | 6.67 | 2.31 | 34.6 |
| Cₘₐₓ | ng/ml | 41.5 | 67.3 | 50.6 | 53.1 | 13.1 | 24.6 |
| AUCₗₐₛₜ | h*ng/ml | 480 | 875 | 726 | 694 | 199 | 28.7 |
| AUC_{Inf} | h*ng/ml | — | — | — | — | — | — |
| AUC_%Extrap_obs | % | — | — | — | — | — | — |
| MRT_{Inf}_obs | h | — | — | — | — | — | — |
| AUCₗₐₛₜ/D | h*mg/mL | 48.0 | 87.5 | 72.6 | 69.4 | 19.9 | 28.7 |
| F | % | 2.51 | 4.57 | 3.79 | 3.62 | 1.04 | 28.7 |

TABLE 8D
Compound A Pharmacokinetics in propylene glycol/Vitamin E TPGS/Water-based Formulation.

| | | Animal | Animal | Animal | | | CV |
|---|---|---|---|---|---|---|---|
| Time (h) | Unit | 1 | 2 | 3 | Mean | SD | (%) |
| T_{1/2} | h | — | 4.33 | — | — | — | — |
| Tₘₐₓ | h | 4.00 | 2.00 | 4.00 | 3.33 | 1.15 | 34.6 |
| Cₘₐₓ | ng/mL | 445 | 375 | 437 | 419 | 38 | 9.14 |
| AUCₗₐₛₜ | h*ng/ml | 4949 | 3428 | 3755 | 4044 | 801 | 19.8 |
| AUC_{Inf} | h*ng/ml | — | 3507 | — | — | — | — |
| AUC_%Extrap_obs | % | — | 2.25 | — | — | — | — |
| MRT_{Inf}_obs | h | — | 6.39 | — | — | — | — |
| AUCₗₐₛₜ/D | h*mg/ml | 495 | 343 | 375 | 404 | 80 | 19.8 |
| F | % | 25.8 | 17.1 | 19.6 | 20.8 | 4.5 | 21.7 |

There were no recorded clinical observations for either compound formulation, suggesting that both formulations were well tolerated.

Without being bound by any particular theory, the results of this study indicate that the propylene glycol-based formulation results in an increase plasma concentration of Compound A relative to a methylcellulose-based formulation and suggest a larger therapeutic window when the compound is orally administered in a propylene glycol-based formulation.

### Rat PK Study #2

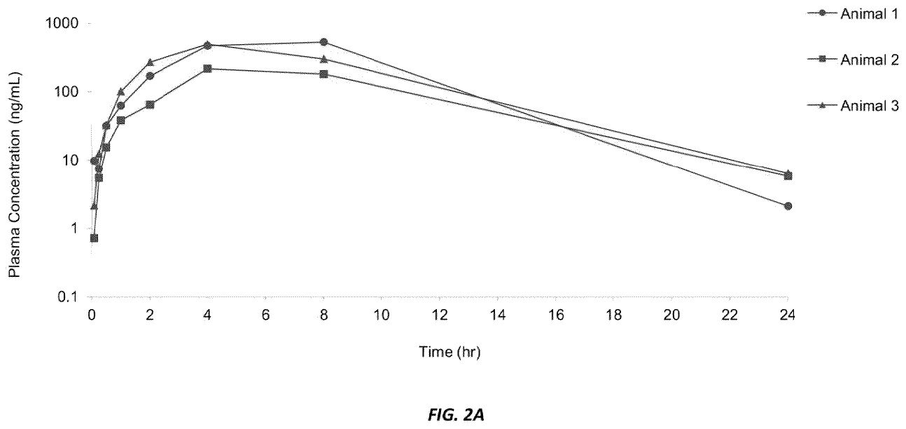

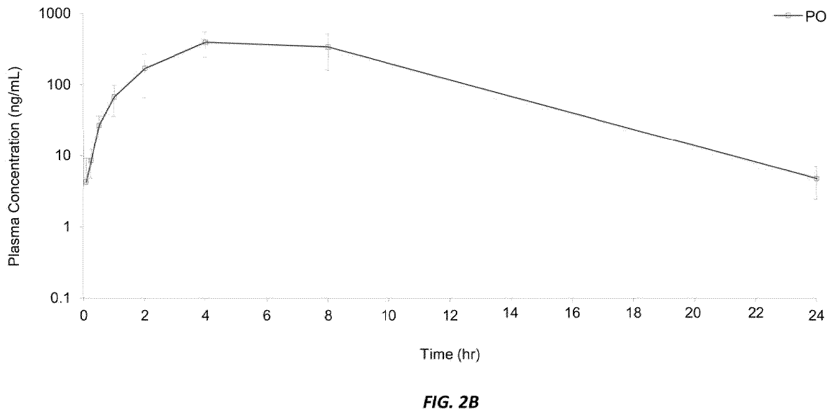

The second rat pharmacokinetic study evaluated the pharmacokinetics of Compound A in SD rats when formulated in a DMSO-based formulation. FIG. 2A illustrates the plasma concentration of Compound A over a 24-hour period following oral administration of Compound A (5 mg/kg) for all subjects tested, and FIG. 2B depicts the mean plasma concentration. Table 9A and 9B further depict the plasma concentration over the 24-hour period (Table 9A), as well as the pharmacokinetic parameters for Compound A when formulated in a DMSO-based solution (Table 9B).

TABLE 9A
Compound A Plasma Concentration in
DMSO/PEG-400/Milli-Q Water-based Formulation.

| Time | Animal 1 | Animal 2 | Animal 3 | Mean | SD | CV |
|---|---|---|---|---|---|---|
| (h) | (ng/ml) | (ng/ml) | (ng/ml) | (ng/ml) | (ng/ml) | (%) |
| 0.083 | 9.76 | 0.719 | 2.15 | 4.21 | 4.86 | 116 |
| 0.25 | 7.42 | 5.44 | 12.5 | 8.5 | 3.7 | 43.3 |
| 0.5 | 31.9 | 15.4 | 32.7 | 26.7 | 9.8 | 36.6 |
| 1 | 62.6 | 38.4 | 101 | 67 | 32 | 47.0 |
| 2 | 170 | 64.8 | 272 | 169 | 103 | 61.2 |
| 4 | 472 | 218 | 496 | 395 | 154 | 39.0 |
| 8 | 532 | 181 | 302 | 338 | 178 | 52.7 |
| 24 | 2.11 | 5.81 | 6.35 | 4.75 | 2.31 | 48.6 |

TABLE 9B
Compound A Pharmacokinetics in DMSO/PEG-400/Milli-Q Water-
based Formulation.

| | | Animal | Animal | Animal | | | CV |
|---|---|---|---|---|---|---|---|
| Time (h) | Unit | 1 | 2 | 3 | Mean | SD | (%) |
| T_{1/2} | h | — | — | — | — | — | — |
| Tₘₐₓ | h | 8.00 | 4.00 | 4.00 | 5.33 | 2.31 | 43.3 |
| Cₘₐₓ | ng/ml | 532 | 218 | 496 | 415 | 172 | 41.4 |
| AUCₗₐₛₜ | h*ng/ml | 7070 | 2643 | 5058 | 4924 | 2216 | 45.0 |
| AUC_{Inf} | h*ng/ml | — | — | — | — | — | — |
| AUC_%Extrap_obs | % | — | — | — | — | — | — |
| MRT_{Inf}_obs | h | — | — | — | — | — | — |
| AUCₗₐₛₜ/D | h*mg/ml | 1414 | 529 | 1012 | 985 | 443 | 45.0 |
| F | % | 81.5 | 30.5 | 58.3 | 56.8 | 25.5 | 45.0 |

There were no abnormalities recorded, and only one animal had soft feces at the 8-hour timepoint, and the loose feces had resolved by the 24-hour timepoint.

### Rat PK Study #3

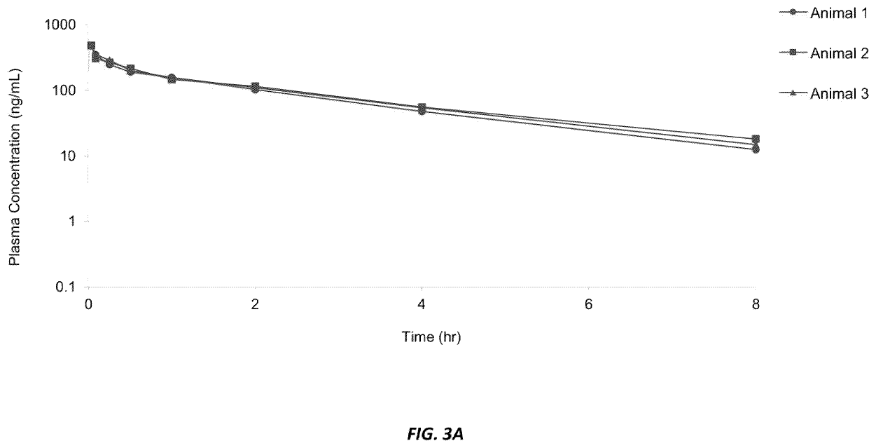

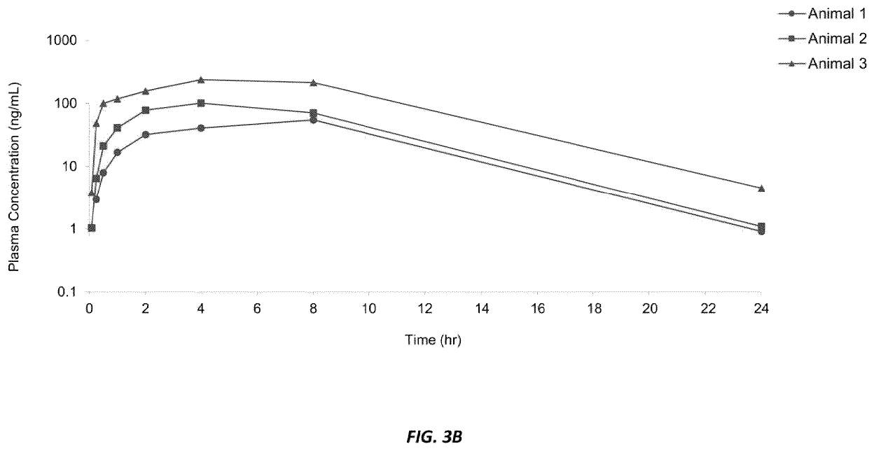

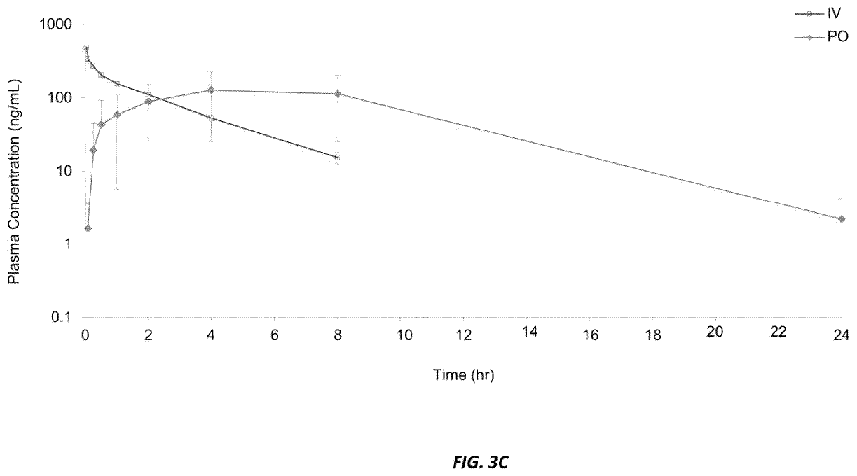

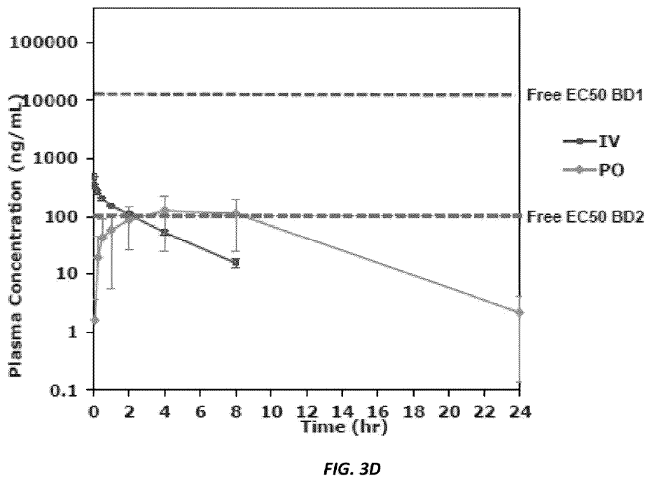

The third rat pharmacokinetic study investigated the pharmacokinetics of ABBV-744 (AbbVie) as a reference point and comparator for Compound A pharmacokinetics. ABBV-744 was formulated in a DM80-based formulation and administered orally and intravenously. FIG. 3A shows the plasma concentration over a 24-hour period after IV administration (1 mg/kg) (Table 10A), whereas FIG. 3B shows plasma concentrations over a 24-hour period after PO administration (5 mg/kg) (Table 10B). Tables 10G and 10D further show the pharmacokinetic parameters after IV and PG administration, respectively. FIG. 3C further shows a comparison of the mean plasma concentration after IV and PO administration. FIG. 3D further depicts the plasma concentration relative to the free EC50 for BDI and the free EC50 for BD2.

TABLE 10A
ABBV-744 IV Administration Plasma Concentration in
DMSO/PEG-400/Milli-Q Water-based Formulation.

| Time | Animal 1 | Animal 2 | Animal 3 | Mean | SD | CV |
|---|---|---|---|---|---|---|
| (h) | (ng/ml) | (ng/ml) | (ng/mL) | (ng/ml) | (ng/ml) | (%) |
| 0.033 | 475 | 484 | 489 | 483 | 7 | 1.47 |
| 0.083 | 351 | 308 | 353 | 337 | 25 | 7.54 |
| 0.25 | 248 | 267 | 289 | 268 | 21 | 7.66 |
| 0.5 | 191 | 217 | 202 | 203 | 13 | 6.42 |
| 1 | 158 | 147 | 158 | 154 | 6 | 4.12 |
| 2 | 103 | 116 | 110 | 110 | 7 | 5.93 |
| 4 | 48.0 | 55.9 | 54.8 | 52.9 | 4.3 | 8.09 |
| 8 | 12.6 | 18.3 | 15.0 | 15.3 | 2.9 | 18.7 |

TABLE 10B
ABBV-744 PO Administration Plasma Concentration
in DMSO/PEG-400/Milli-Q Water-based Formulation.

| Time | Animal 1 | Animal 2 | Animal 3 | Mean | SD | CV |
|---|---|---|---|---|---|---|
| (h) | (ng/ml) | (ng/ml) | (ng/ml) | (ng/ml) | (ng/mL) | (%) |
| 0.083 | 0.00 | 1.03 | 3.81 | 1.61 | 2.0 | 122 |
| 0.25 | 2.95 | 6.42 | 48.5 | 19.3 | 25.4 | 131 |
| 0.5 | 7.91 | 21.1 | 100 | 43 | 50 | 116 |
| 1 | 16.7 | 41.0 | 118 | 59 | 53 | 90.3 |
| 2 | 31.9 | 78.0 | 157 | 89 | 63 | 71.1 |
| 4 | 40.6 | 101 | 239 | 127 | 102 | 80.2 |
| 8 | 54.6 | 70.6 | 214 | 113 | 88 | 77.6 |
| 24 | 0.909 | 1.09 | 4.52 | 2.17 | 2.03 | 93.6 |

TABLE 10C
ABBV-744 IV Administration Pharmacokinetics in DMSO/PEG-
400/Milli-Q Water-based Formulation.

| | | Animal | | | | | |
|---|---|---|---|---|---|---|---|
| Time (h) | Unit | 1 | Animal 2 | Animal 3 | Mean | SD | CV (%) |
| Cl_obs | mL/min/kg | 24.9 | 22.2 | 22.9 | 23.3 | 1.4 | 5.99 |
| T_{1/2} | h | 1.99 | 2.28 | 2.09 | 2.12 | 0.15 | 6.93 |
| C₀ | ng/mL | 580 | 652 | 606 | 613 | 37 | 5.97 |
| AUCₗₐₛₜ | h*ng/ml | 633 | 690 | 683 | 668 | 31 | 4.64 |
| AUC_{Inf} | h*ng/ml | 669 | 750 | 728 | 716 | 42 | 5.85 |
| AUC_%Extrap_obs | % | 5.41 | 8.03 | 6.23 | 6.56 | 1.34 | 20.5 |
| MRT_{Inf}_obs | h | 2.47 | 2.88 | 2.61 | 2.65 | 0.21 | 7.87 |
| AUCₗₐₛₜ/D | h*mg/mL | 633 | 690 | 683 | 668 | 31 | 4.64 |
| Vₛₛ_obs | L/kg | 3.69 | 3.84 | 3.59 | 3.71 | 0.12 | 3.37 |

TABLE 10D
ABBV-744 PO Administration Pharmacokinetics in DMSO/PEG-400/Milli-Q Water-based Formulation.

| | | Animal | Animal | Animal | | | CV |
|---|---|---|---|---|---|---|---|
| Time (h) | Unit | 1 | 2 | 3 | Mean | SD | (%) |
| T_{1/2} | h | — | — | — | — | — | — |
| Tₘₐₓ | h | 8.00 | 4.00 | 4.00 | 5.33 | 2.31 | 43.3 |
| Cₘₐₓ | ng/ml | 54.6 | 101 | 239 | 132 | 96 | 72.9 |
| AUCₗₐₛₜ | h*ng/ml | 739 | 1175 | 3265 | 1726 | 1350 | 78.2 |
| AUC_{Inf} | h*ng/ml | — | — | — | — | — | — |
| AUC_%Extrap_obs | % | — | — | — | — | — | — |
| MRT_{Inf}_obs | h | — | — | — | — | — | — |
| AUCₗₐₛₜ/D | h*mg/mL | 148 | 235 | 653 | 345 | 270 | 78.2 |
| F | % | 22.1 | 35.2 | 97.7 | 51.7 | 40.4 | 78.2 |

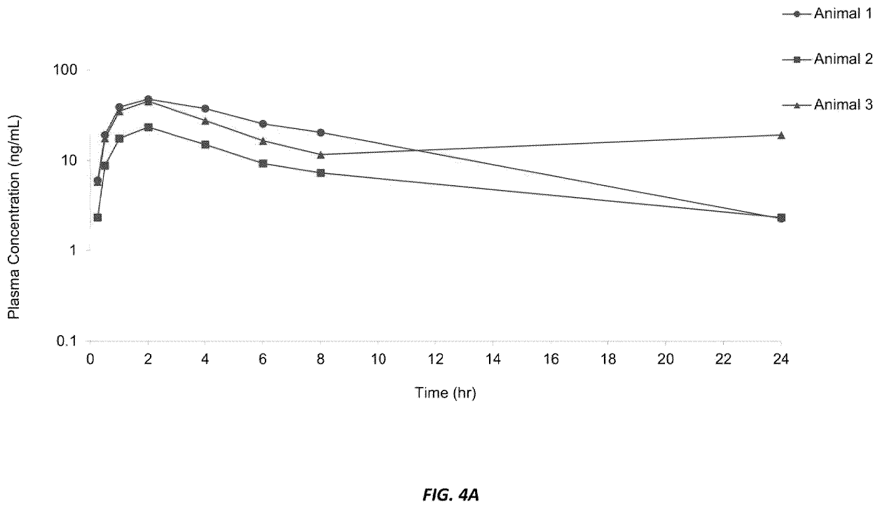

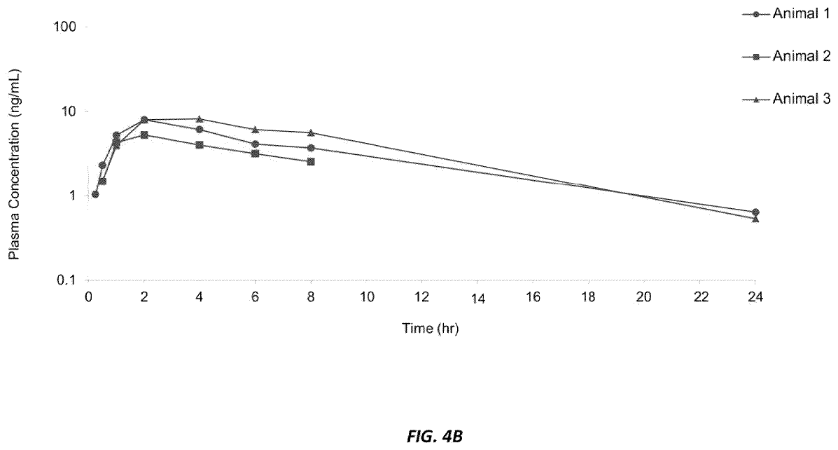

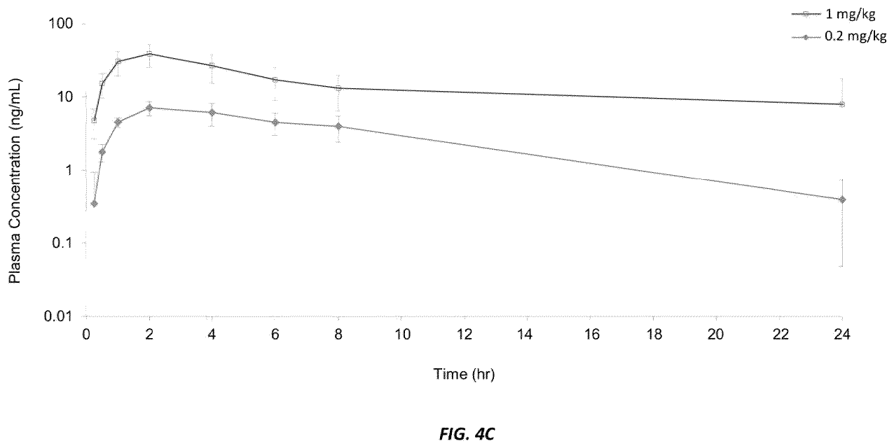

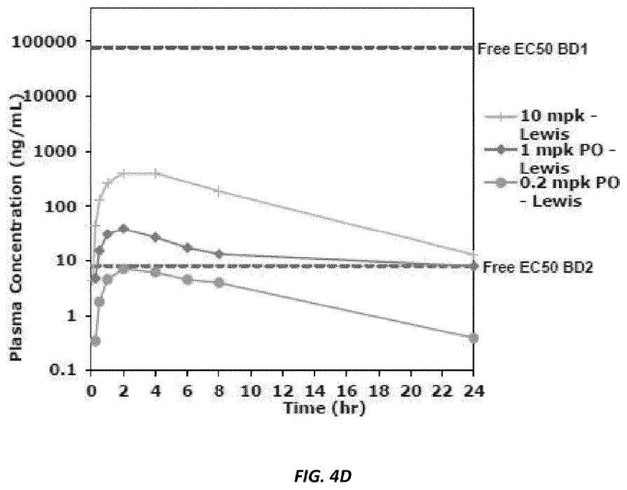

No clinical observations were noted for either delivery route. Comparison of the pharmacokinetic profile of Compound A, and ABBV-744 indicates that Compound A exhibits robust exposure above the free EC50 BD2 BRD4 at both the 1 mg/kg and 10 mg/kg doses for 24 hours, whereas ABBV-744 exposure above the free EC50 BD2 BRD4 was minimal when administered at a 5 mg/kg dose (compare FIG. 3D (ABBV-744) with FIG. 4D (Compound A)). Without being bound by any particular theory, the data suggest that Compound A exhibits a larger therapeutic window that ABBV-744.

### Rat PK Study #4

The fourth rat pharmacokinetic study evaluated the pharmacokinetics of Compound A when orally delivered at two different doses in a propylene glycol-based formulation. FIG. 4A and Table 21A illustrate the plasma concentration of Compound A (1 mg/kg) over the 24-hour study period and FIG. 4B and Table 11B illustrate the plasma concentration of Compound A (0.2 mg/kg) over the 24-hour study period. FIG. 4C provides a comparison of the mean plasma concentration for the two doses. FIG. 4D depicts the plasma concentration for a range of doses of Compound A (0.2 mg/kg, 1 mg/kg, and 10 mg/kg) in relation to the free EC50 BD1 (84.7 μg/mL) and free EC50 BD2 BRD4 level (8.8 ng/mL) which had been previously determined. Tables 11C and 11 D provide the pharmacokinetic parameters for Compound A at the 1 mg/kg and 0.2 mg/kg doses, respectively.

TABLE 11A
Compound A 1 mg/kg PO Administration Plasma Concentration in
Propylene Glycol-based Formulation.

| Time | Animal 1 | Animal 2 | Animal 3 | Mean | SD | CV |
|---|---|---|---|---|---|---|
| (h) | (ng/ml) | (ng/ml) | (ng/ml) | (ng/ml) | (ng/ml) | (%) |
| 0.25 | 6.05 | 2.36 | 5.82 | 4.74 | 2.07 | 43.6 |
| 0.5 | 19.1 | 8.80 | 17.6 | 15.2 | 5.6 | 36.7 |
| 1 | 38.8 | 17.5 | 35.0 | 30.4 | 11.4 | 37.3 |
| 2 | 47.5 | 23.3 | 44.9 | 38.6 | 13.3 | 34.4 |
| 4 | 37.4 | 15.0 | 27.5 | 26.6 | 11.2 | 42.1 |
| 6 | 25.4 | 9.27 | 16.5 | 17.1 | 8.1 | 47.4 |
| 8 | 20.4 | 7.30 | 11.6 | 13.1 | 6.7 | 51.0 |
| 24 | 2.29 | 2.35 | 19.1 | 7.9 | 9.7 | 122 |

TABLE 11B
Compound A 0.2 mg/kg PO Administration Plasma
Concentration in Propylene Glycol-based Formulation.

| Time | Animal 1 | Animal 2 | Animal 3 | Mean | SD | CV |
|---|---|---|---|---|---|---|
| (h) | (ng/mL) | (ng/ml) | (ng/ml) | (ng/ml) | (ng/ml) | (%) |
| 0.25 | 1.03 | 0.00 | 0.00 | 0.34 | 0.59 | 173 |
| 0.5 | 2.31 | 1.48 | 1.51 | 1.77 | 0.47 | 26.6 |
| 1 | 5.24 | 4.31 | 3.98 | 4.51 | 0.65 | 14.5 |
| 2 | 7.99 | 5.29 | 8.00 | 7.09 | 1.56 | 22.0 |
| 4 | 6.12 | 4.02 | 8.20 | 6.11 | 2.09 | 34.2 |
| 6 | 4.13 | 3.18 | 6.13 | 4.48 | 1.51 | 33.6 |
| 8 | 3.71 | 2.55 | 5.63 | 3.96 | 1.56 | 39.2 |
| 24 | 0.635 | 0.00 | 0.532 | 0.389 | 0.341 | 87.6 |

TABLE 11C
Compound A 1 mg/kg PO Administration Pharmacokinetic Parameters in Propylene Glycol-based Formulation.

| | | Animal | Animal | Animal | | | CV |
|---|---|---|---|---|---|---|---|
| Time (h) | Unit | 1 | 2 | 3 | Mean | SD | (%) |
| T_{1/2} | h | 5.14 | 9.35 | NA | 7.24 | NA | NA |
| Tₘₐₓ | h | 2.00 | 2.00 | 2.00 | 2.00 | 0.00 | 0.00 |
| Cₘₐₓ | ng/ml | 47.5 | 23.3 | 44.9 | 38.6 | 13.3 | 34.4 |
| AUCₗₐₛₜ | h*ng/ml | 437 | 185 | 447 | 356 | 148 | 41.6 |
| AUC_{Inf} | h*ng/ml | 454 | 217 | NA | 335 | NA | NA |
| AUC_%Extrap_obs | % | 3.74 | 14.6 | NA | 9.2 | NA | NA |
| MRT_{Inf}_obs | h | 7.10 | 11.4 | NA | 9.3 | NA | NA |
| AUCₗₐₛₜ/D | (ng · h/mL)/ | 437 | 185 | 447 | 356 | 148 | 41.6 |
| | (mg/kg) | | | | | | |
| F | % | NA | NA | NA | NA | NA | NA |

The values of T_{1/2}, AUC_{inf}, AUC_{%Extrap}_obs, MRT_{Inf}_obs were reported as NA since Rsq < 0.85.

TABLE 11D
Compound A 0.2 mg/kg PO Administration Pharmacokinetic Parameters in Propylene Glycol-based Formulation.

| | | Animal | Animal | Animal | | | CV |
|---|---|---|---|---|---|---|---|
| Time (h) | Unit | 1 | 2 | 3 | Mean | SD | (%) |
| T_{1/2} | h | 6.51 | 6.09 | 4.94 | 5.85 | 0.81 | 13.9 |
| Tₘₐₓ | h | 2.00 | 2.00 | 4.00 | 2.67 | 1.15 | 43.3 |
| Cₘₐₓ | ng/mL | 7.99 | 5.29 | 8.20 | 7.16 | 1.62 | 22.7 |
| AUCₗₐₛₜ | h*ng/ml | 76.0 | 28.7 | 99.1 | 67.9 | 35.9 | 52.9 |
| AUC_{Inf} | h*ng/ml | 82.0 | 51.1 | 103 | 79 | 26 | 33.2 |
| AUC_%Extrap_obs | % | 7.28 | 43.9 | 3.68 | 18.3 | 22.2 | 122 |
| MRT_{Inf}_obs | h | 8.72 | 9.52 | 7.69 | 8.65 | 0.92 | 10.6 |
| AUCₗₐₛₜ/D | (ng · h/mL)/(mg/kg) | 380 | 143 | 496 | 340 | 180 | 52.9 |
| F | % | NA | NA | NA | NA | NA | NA |

The values of T_{1/2}, AUC_{inf}, AUC_{%Extrap}_obs, MRT_{Inf}_obs were reported as NA since Rsq < 0.85.

There were no abnormal clinical symptoms observed during the course of the study. Without being bound by any particular theory, Compound A, in a propylene glycol-based formulation appears to be well-tolerated.

TABLE 11E
Outline of the Four Rat pK Studies.

| | | | | Dose |
|---|---|---|---|---|
| Study | Type | Formulation | Route | mg/kg |
| 1 | Lewis | 1% methylcellulose | PO | 10 |
| | Lewis | 20% PG/20% Vit E/60% water | PO | 10 |
| 2 | SD | 5% DMSO, 40% PEG-400, 55% | PO | 5 |
| | | Milli-Q water | | |
| 3 | SD | 5% DMSO, 40% PEG-400, 55% | PO | 5 |
| | | Milli-Q water | | |
| | SD | 5% DMSO, 40% PEG-400, 55% | IV | 1 |
| | | Milli-Q water | | |
| 4 | Lewis | 20% PG/20% Vit E/60% water | PO | 0.2 |
| | Lewis | 20% PG/20% Vit E/60% water | PO | 1 |

### Dog PK Studies

Four studies were undertaken with Compound A in dogs: one in a propylene glycol based formulation (20% propylene glycol/20% Vitamin E TPGS/60% water) (Dog PK study #1), one in a HPPCD based formulation (HPPCD (20% solution w/v)/DMSO (99/1)) (Dog PK study #2), one in a 5% DMSO/15% PEG-400/80% (10% VE-TPGS) formulation (Dog PK study #3), and one in a 5% DMSO/15% PEG-400/80% (10% VE-TPGS) (Dog PK study #4). Overall, the studies show that Compound A is IV and orally available and well tolerated. Whilst various transient clinical observations were noted in certain animals in IV study 1, they were not observed in IV study 2 save for one specific exception of loose feces at 8 hours in one animal, indicating that, without being bound by any theory, the clinical observations may be ascribed to the propylene glycol-based formulation rather than Compound A. In addition, the number of recorded clinical observations in the PG arm of study 1 was less than in the IV arm and limited to “soft stool” and to “loose feces,” with the exception of just one of the animals that received the highest dose in the 5 day Dog PK study #3 and was found dead on day 6.

### Dog PK Study #1

In Dog PK study #1, animals were administered solutions with different concentrations of Compound A either intravenously (group 1) or orally (group 2) as outlined in Table 3B of Experimental Method B.

Tables 12A-12B depict plasma concentration of Compound A in animals from Dog PK study #1 receiving Compound A via intravenous administration. Tables 12C-12D depict plasma concentration of Compound A in animals from Dog PK study #1 receiving Compound A via oral administration.

TABLE 12A
Plasma concentration of Compound A in
Dog PK study #1 after IV administration

Concentration (ng/mL)

| Time (h) | Test Animal 1 | Test Animal 2 | Test Animal 3 |
|---|---|---|---|
| 0.083 | 1480 | 1360 | 1600 |
| 0.25 | 1130 | 959 | 1310 |
| 0.5 | 971 | 744 | 1020 |
| 1 | 802 | 577 | 912 |
| 2 | 594 | 422 | 741 |
| 4 | 285 | 145 | 448 |
| 8 | 139 | 58.7 | 309 |
| 24 | 19.2 | 9.04 | 88.0 |

TABLE 12B
Average plasma concentration of Compound A
in Dog PK study #1 after IV administration

| Time (h) | Mean (ng/mL) | SD (ng/mL) | CV (%) |
|---|---|---|---|
| 0.083 | 1480 | 120 | 8.11 |
| 0.25 | 1133 | 176 | 15.5 |
| 0.5 | 912 | 147 | 16.2 |
| 1 | 764 | 171 | 22.4 |
| 2 | 586 | 160 | 27.3 |
| 4 | 293 | 152 | 51.8 |
| 8 | 169 | 128 | 75.7 |
| 24 | 38.7 | 43.0 | 111 |

TABLE 12C
Plasma concentration of Compound A in Dog
PK study #1 after oral administration

Concentration (ng/mL)

| Time (h) | Test Animal 1 | Test Animal 2 | Test Animal 3 |
|---|---|---|---|
| 0.25 | 25.1 | 19.4 | 35.5 |
| 0.5 | 147 | 99.8 | 133 |
| 1 | 354 | 176 | 349 |
| 2 | 498 | 228 | 580 |
| 4 | 450 | 200 | 456 |
| 8 | 182 | 51.0 | 135 |
| 24 | 19.2 | 62.0 | 8.40 |

TABLE 12D
Average plasma concentration of Compound A
in Dog PK study #1 after oral administration

| Time (h) | Mean (ng/mL) | SD (ng/mL) | CV (%) |
|---|---|---|---|
| 0.25 | 26.7 | 8.2 | 30.6 |
| 0.5 | 127 | 24 | 19.1 |
| 1 | 293 | 101 | 34.6 |
| 2 | 435 | 184 | 42.3 |
| 4 | 369 | 146 | 39.6 |
| 8 | 123 | 66 | 54.1 |
| 24 | 29.9 | 28.3 | 94.9 |

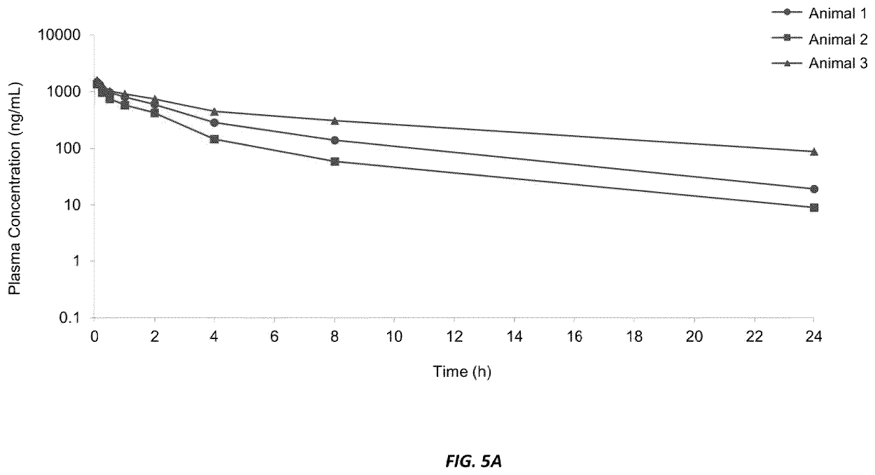

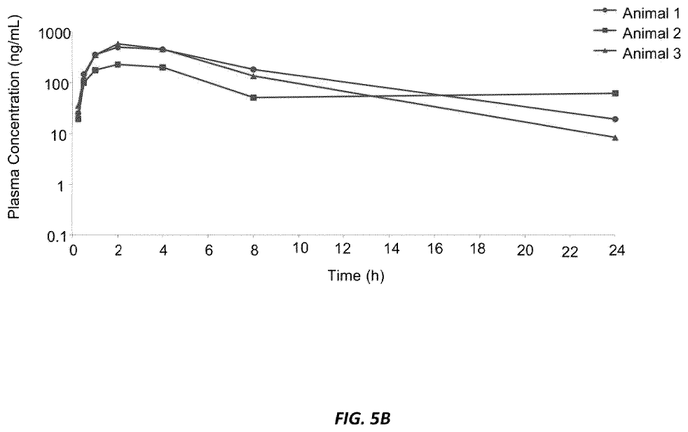

FIGS. 5A-5C relate to Dog PIK study #1. FIG. 5A shows a graph plotting the plasma concentration of Compound A in group 1 as a function of time. Plasma concentrations decreased similarly in all animals over the first 2 h post-administration in animals receiving Compound A via intravenous injection. Animals 1 and 2 exhibited similar decreasing plasma levels of Compound A over the remaining timepoints, whereas animal 3 maintained higher plasma concentrations of Compound A over all timepoints, but plasma concentrations of Compound A did decrease over all timepoints.

FIG. 5B shows a graph plotting the plasma concentration of Compound A in group 2 as a function of time. Plasma concentrations of Compound A increased for the first 2 h in all animals receiving Compound A via oral administration. Animals 1 and 3 exhibited a similar decrease in Compound A plasma concentration over the remaining timepoints, whereas animal 2 maintained a relatively similar plasma concentration at the 8 h and 24 h timepoints following oral administration.

FIG. 5C shows a graph plotting the mean plasma concentration of Compound A in groups 1 and 2, respectively, as a function of time.

Tables 12E and 12F show the pharmacokinetic profiles of Compound A after intravenous administration and Tables 12G and 12H show the pharmacokinetic profiles of Compound A after oral administration (Table 12B) in beagle dogs as outlined in Table 3B in Experimental Method B.

TABLE 12E
PK profile of Compound A following intravenous
administration in Dog PK study #1.

| | | Test | Test | |
|---|---|---|---|---|
| Pharmacokinetic | | Animal | Animal | Test |
| Parameter | Unit | 1 | 2 | Animal 3 |
| CI_obs | mL/min/kg | 3.41 | 5.66 | 1.86 |
| T_{1/2} | h | 5.26 | 5.23 | 8.60 |
| C₀ | ng/mL | 1692 | 1618 | 1767 |
| AUCₗₐₛₜ | h*ng/mL | 4746 | 2876 | 7862 |
| AUC_{lnf} | h*ng/mL | 4892 | 2944 | 8955 |
| AUC_{—%Extrap}_obs | % | 2.98 | 2.32 | 12.2 |
| MRT_{lnf}_obs | h | 5.21 | 4.15 | 10.0 |
| AUCₗₐₛₜ/D | (ng · h/mL)/(mg/kg) | 4746 | 2876 | 7862 |
| Vₛₛ_obs | L/kg | 1.06 | 1.41 | 1.12 |

TABLE 12F
Average PK profile of Compound A following intravenous
administration in Dog PK study #1.

| Pharmacokinetic | | | | |
|---|---|---|---|---|
| Parameter | Unit | Mean | SD | CV (%) |
| CI_obs | mL/min/kg | 3.64 | 1.91 | 52.4 |
| T_{1/2} | h | 6.37 | 1.94 | 30.5 |
| C₀ | ng/mL | 1692 | 75 | 4.41 |
| AUCₗₐₛₜ | h*ng/mL | 5162 | 2519 | 48.8 |
| AUC_{lnf} | h*ng/mL | 5597 | 3067 | 54.8 |
| AUC_{—%Extrap}_obs | % | 5.8 | 5.5 | 94.7 |
| MRT_{lnf}_obs | h | 6.5 | 3.1 | 48.6 |
| AUCₗₐₛₜ/D | (ng · h/mL)/(mg/kg) | 5162 | 2519 | 48.8 |
| Vₛₛ_obs | L/kg | 1.20 | 0.19 | 15.5 |

TABLE 12G
PK profile of Compound A following oral
administration in Dog PK study #1.

| | | Test | Test | |
|---|---|---|---|---|
| Pharmacokinetic | | Animal | Animal | Test |
| Parameter | Unit | 1 | 2 | Animal 3 |
| T_{1/2} | h | 4.54 | NA | 3.61 |
| Tₘₐₓ | h | 2.00 | 2.00 | 2.00 |
| Cₘₐₓ | ng/mL | 498 | 228 | 580 |
| AUCₗₐₛₜ | h*ng/mL | 4398 | 2122 | 3976 |
| AUC_{lnf} | h*ng/mL | 4523 | NA | 4019 |
| AUC_{—%Extrap}_obs | % | 2.78 | NA | 1.09 |
| MRT_{lnf}_obs | h | 6.47 | NA | 5.27 |
| AUCₗₐₛₜ/D | (ng · h/mL)/(mg/kg) | 880 | 424 | 795 |
| F | % | 16.2 | 8.22 | 14.4 |

TABLE 12H
Average PK profile of Compound A following
oral administration in Dog PK study #1.

| Pharmacokinetic | | | | CV |
|---|---|---|---|---|
| Parameter | Unit | Mean | SD | (%) |
| T_{1/2} | h | 4.07 | NA | NA |
| Tₘₐₓ | h | 2.00 | 0.00 | 0.00 |
| Cₘₐₓ | ng/mL | 435 | 184 | 42.3 |
| AUCₗₐₛₜ | h*ng/mL | 3498 | 1210 | 34.6 |
| AUC_{lnf} | h*ng/mL | 4271 | NA | NA |
| AUC_{—%Extrap}_obs | % | 1.93 | NA | NA |
| MRT_{lnf}_obs | h | 5.87 | NA | NA |
| AUCₗₐₛₜ/D | (ng · h/mL)/(mg/kg) | 700 | 242 | 34.6 |
| F | % | 12.9 | 4.2 | 32.2 |

### Dog PK Study #2

In Dog PK study #2, animals were administered a solution of Compound A intravenously as outlined in Table 3C of Experimental Method B.

Tables 13A and 13B provide the plasma concentration of Compound A, over time in animals in Dog PK study #2.

TABLE 13A
Plasma concentration of Compound A in Dog PK study #2

Concentration (ng/mL)

| Time (h) | Test Animal 1 | Test Animal 2 | Test Animal 3 |
|---|---|---|---|
| 0.083 | 479 | 464 | 396 |
| 0.25 | 540 | 440 | 370 |
| 0.5 | 405 | 305 | 273 |
| 1 | 331 | 281 | 195 |
| 2 | 323 | 190 | 184 |
| 4 | 305 | 113 | 110 |
| 8 | 207 | 37.4 | 54.1 |
| 24 | 43.4 | 10.5 | 24.1 |

TABLE 13B
Average plasma concentration of Compound A in Dog PK study #2

| Time (h) | Mean (ng/mL) | SD (ng/mL) | CV (%) |
|---|---|---|---|
| 0.083 | 446 | 44 | 9.91 |
| 0.25 | 450 | 85 | 19.0 |
| 0.5 | 328 | 69 | 21.0 |
| 1 | 269 | 69 | 25.6 |
| 2 | 232 | 79 | 33.8 |
| 4 | 176 | 112 | 63.5 |
| 8 | 100 | 93 | 93.9 |
| 24 | 26.0 | 16.5 | 63.6 |

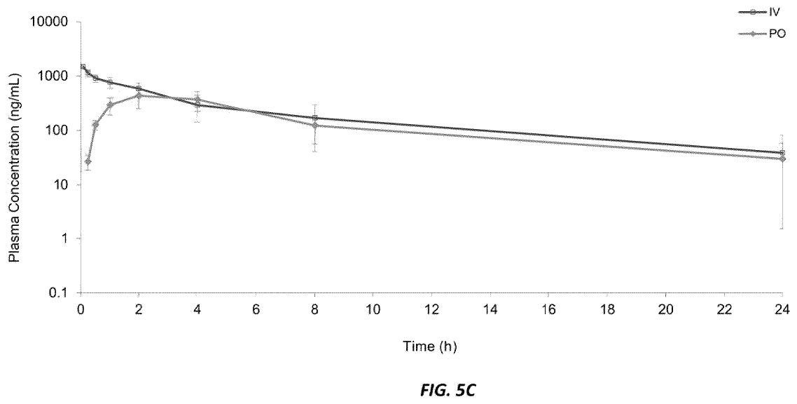

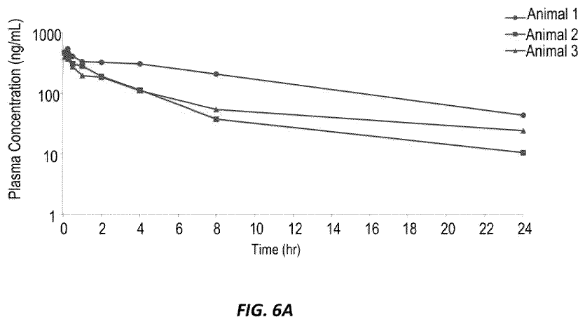

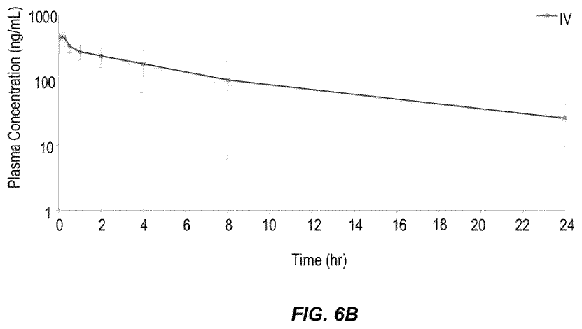

FIGS. 6A-6B show the plasma concentration of Compound A in animals (receiving Compound A via intravenous injection) as a function of time in PK study 2 in a HPβCD based formulation. Plasma concentrations decreased similarly over the first 2 h post-administration in all animals receiving Compound A. Animals 2 and 3 exhibited similar decreasing plasma levels of Compound A over the remaining timepoints, whereas animal 1 maintained higher plasma concentrations of Compound A over all timepoints, but plasma concentrations of Compound A did decrease over all timepoints.

Tables 13C and 13D show the pharmacokinetic profiles of Compound A from PK study 2 with the HPβCD based formulation.

TABLE 13C
PK study pharmacokinetic parameters in Dog PK study #2.

| | | Test | Test | |
|---|---|---|---|---|
| Pharmacokinetic | | Animal | Animal | Test |
| Parameter | Unit | 1 | 2 | Animal 3 |
| CI_obs | mL/min/kg | 3.43 | 9.95 | 8.00 |
| T_{1/2} | h | 7.11 | 6.45 | 10.1 |
| C₀ | ng/mL | 479 | 476 | 410 |
| AUCₗₐₛₜ | h*ng/mL | 4409 | 1577 | 1732 |
| AUC_{lnf} | h*ng/mL | 4854 | 1674 | 2083 |
| AUC_{—%Extrap}_obs | % | 9.17 | 5.83 | 16.8 |
| MRT_{lnf}_obs | h | 9.30 | 6.26 | 11.8 |
| AUCₗₐₛₜ/D | (ng · h/mL)/(mg/kg) | 4409 | 1577 | 1732 |
| Vₛₛ_obs | L/kg | 1.91 | 3.74 | 5.67 |

TABLE 13D
PK study 2 pharmacokinetic parameters in Dog PK study #2.

| Pharmacokinetic | | | | |
|---|---|---|---|---|
| Parameter | Unit | Mean | SD | CV (%) |
| CI_obs | mL/min/kg | 7.13 | 3.35 | 46.9 |
| T_{1/2} | h | 7.9 | 1.9 | 24.7 |
| C₀ | ng/mL | 455 | 39 | 8.65 |
| AUCₗₐₛₜ | h*ng/mL | 2573 | 1592 | 61.9 |
| AUC_{lnf} | h*ng/mL | 2870 | 1730 | 60.3 |
| AUC_{—%Extrap}_obs | % | 10.6 | 5.7 | 53.2 |
| MRT_{lnf}_obs | h | 9.1 | 2.8 | 30.4 |
| AUCₗₐₛₜ/D | (ng · h/mL)/(mg/kg) | 2573 | 1592 | 61.9 |
| Vₛₛ_obs | L/kg | 3.77 | 1.88 | 49.7 |

Without being bound by any particular theory, animals exhibited fewer clinical observations in response to Compound A when prepared in HPPCD relative to Compound A prepared in 20% propylene glycol/20% Vitamin E TPGS/60% water. Without being bound by any particular theory, these studies show that Compound A is capable of being administered orally and intravenously and well tolerated. Additionally, when Compound A was prepared in HPPCD, some animals exhibited fewer reactions shortly after administration of Compound A. Without being bound by any particular theory, this also suggests that the clinical observations noted in PK study 1 may be a result of, either in part or in its entirety, the formulation Compound A was prepared in (i.e., 20% propylene glycol/20% Vitamin E TPGS/60% water).

### Dog PK Study #3

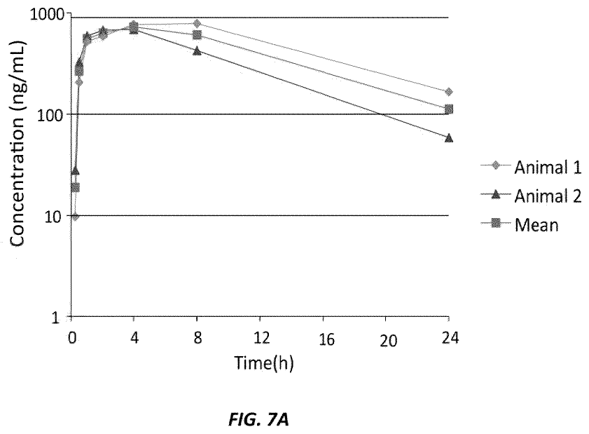

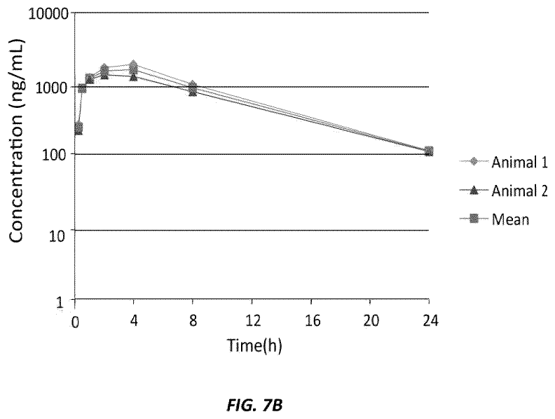

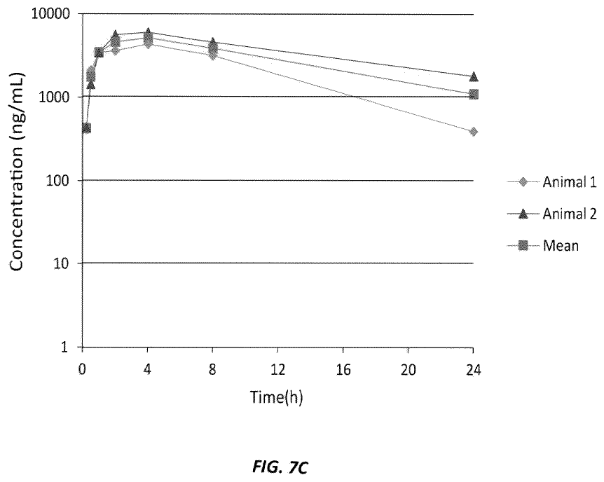

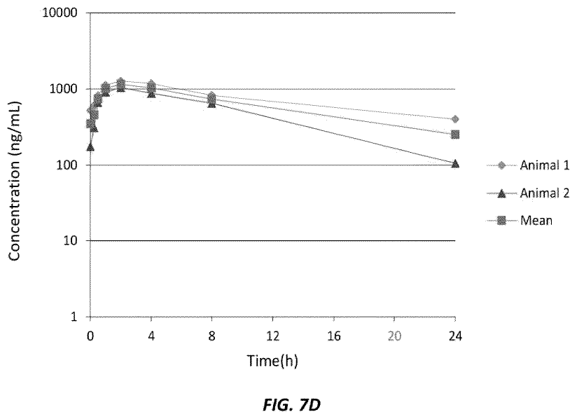

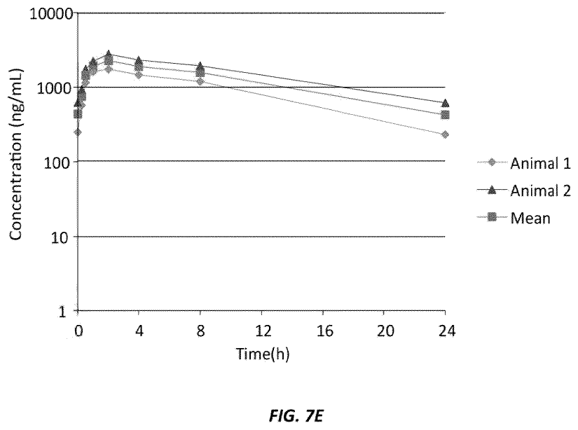

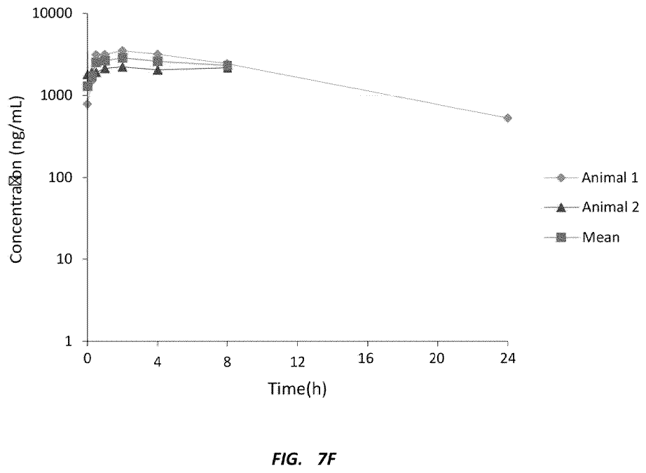

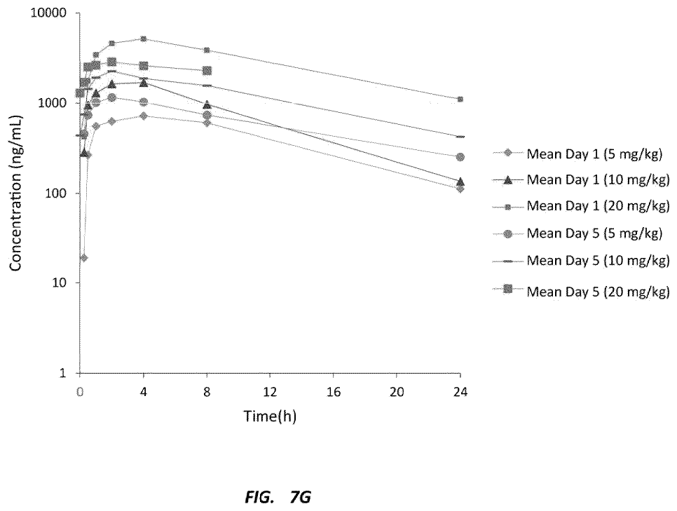

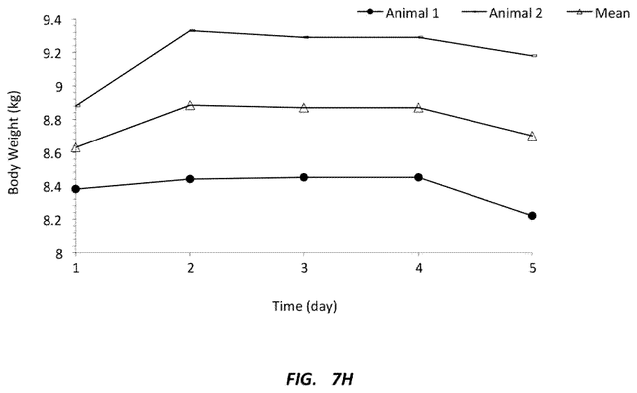

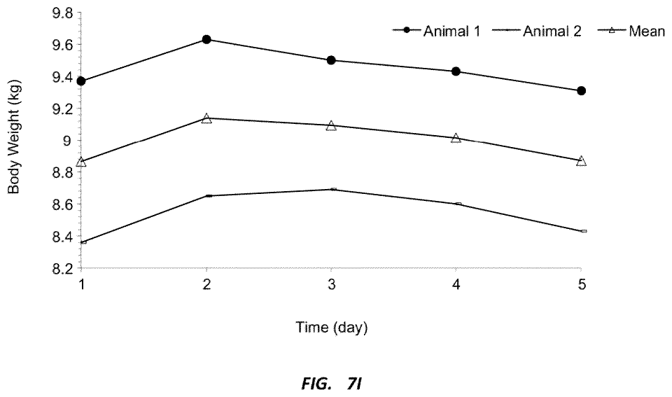

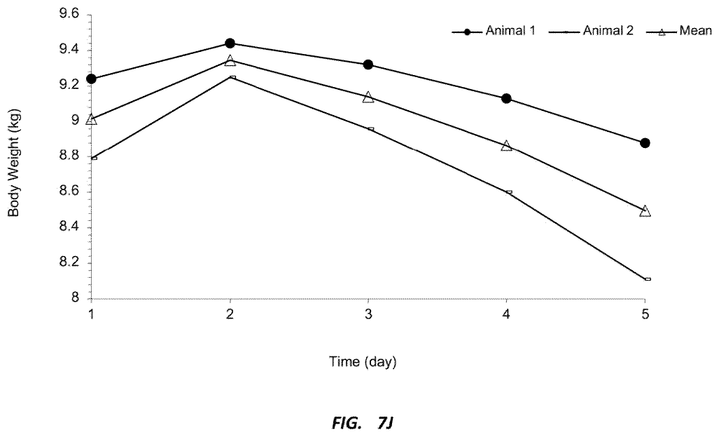

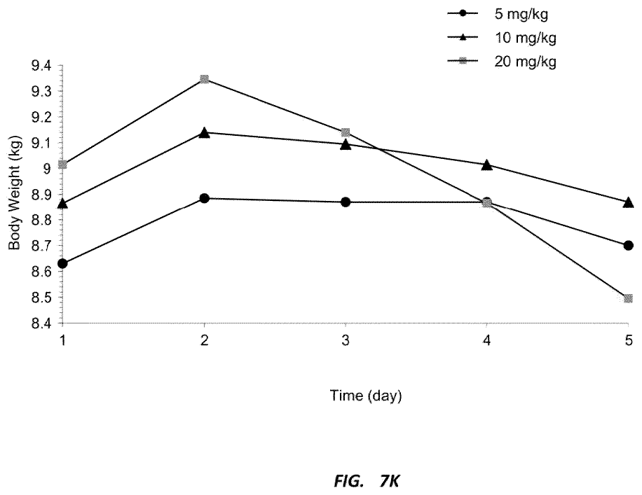

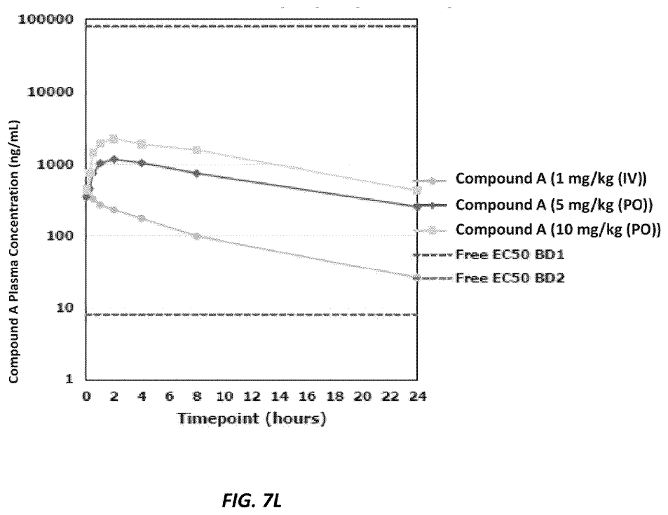

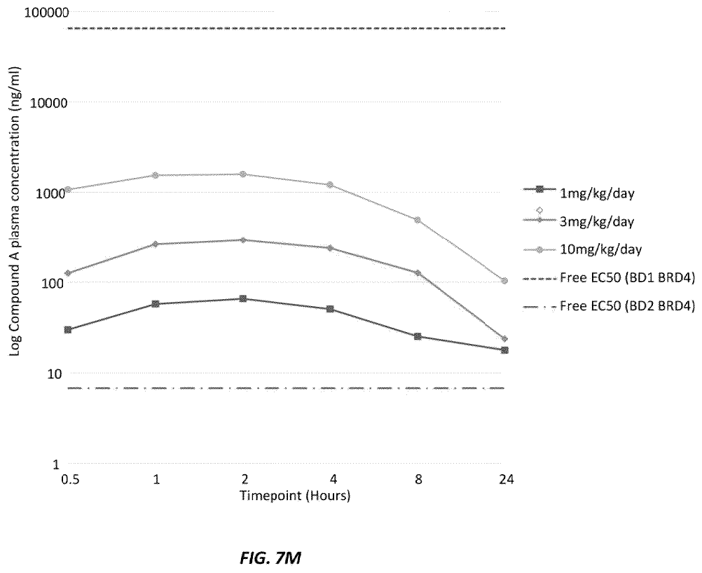

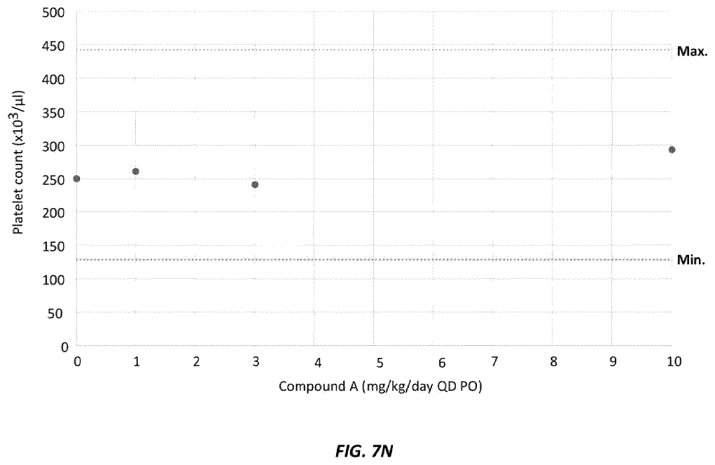

The pharmacokinetics of Compound A was assessed in beagle dogs in Dog PK study #3 as described in Experimental Method B. The plasma concentration of Compound A at three different doses (5 mg/kg, 10 mg/kg, and 20 mg/kg) after 1 day (FIG. 7A-7C, respectively; Table 14A)) and 5 days (FIG. 7D-7F, respectively; Table 14B) was assessed. FIG. 7G shows the mean plasma concentration for each dose and time point. Pharmacokinetic parameters for the different doses are further depicted in Table 14C for the day 5 assessment, and in Table 14D for the day 2 assessment. Table 14E shows dose proportionality for administration of Compound A on day 1, and Table 14F shows the accumulative index of Compound A. Table 14G and FIGS. 5H-5K further show changes in body weight following administration of Compound A. FIG. 7H shows body weight change after administration of the 5 mg/kg dose, FIG. 7I shows body weight change after administration of the 10 mg/kg dose, FIG. 7J shows body weight change after administration of the 20 mg/kg dose, and FIG. 7K shows a comparison of body weight changes for the three doses.

TABLE 14A
Compound A Plasma Concentration at Day 1 in Dog PK study #3.

| | 5 mg/kg dose | | | 10 mg/kg dose | | | 20 mg/kg dose | | |
|---|---|---|---|---|---|---|---|---|---|
| | Animal | Animal | | Animal | Animal | | Animal | Animal | |
| | 1 | 2 | Mean | 1 | 2 | Mean | 1 | 2 | Mean |
| 0 | BQL | BQL | — | BQL | BQL | — | BQL | BQL | ND |
| 0.25 | 9.79 | 28.1 | 18.9 | 310 | 258 | 284 | 413 | 438 | 426 |
| 0.5 | 206 | 326 | 266 | 948 | 954 | 951 | 2090 | 1440 | 1765 |
| 1 | 523 | 590 | 557 | 1330 | 1260 | 1295 | 3430 | 3440 | 3435 |
| 2 | 587 | 671 | 629 | 1820 | 1450 | 1635 | 3620 | 5590 | 4605 |
| 4 | 765 | 686 | 726 | 2010 | 1380 | 1695 | 4330 | 6000 | 5165 |
| 8 | 785 | 424 | 605 | 1080 | 860 | 970 | 3170 | 4570 | 3870 |
| 24 | 166 | 58.7 | 112 | 138 | 134 | 136 | 394 | 1810 | 1102 |

TABLE 14B
Compound A Plasma Concentration at Day 5 in Dog PK study #3.

| | 5 mg/kg dose | | | 10 mg/kg dose | | | 20 mg/kg dose | | |
|---|---|---|---|---|---|---|---|---|---|
| | Animal | Animal | | Animal | Animal | | Animal | Animal | |
| | 1 | 2 | Mean | 1 | 2 | Mean | 1 | 2 | Mean |
| 0 | 522 | 175 | 349 | 250 | 624 | 437 | 788 | 1800 | 1294 |
| 0.25 | 608 | 309 | 459 | 571 | 931 | 751 | 1500 | 1910 | 1705 |
| 0.5 | 814 | 664 | 739 | 1150 | 1730 | 1440 | 3130 | 1910 | 2520 |
| 1 | 1120 | 910 | 1015 | 1610 | 2220 | 1915 | 3150 | 2130 | 2640 |
| 2 | 1270 | 1040 | 1155 | 1740 | 2780 | 2260 | 3510 | 2220 | 2865 |
| 4 | 1180 | 878 | 1029 | 1460 | 2310 | 1885 | 3160 | 2040 | 2600 |
| 8 | 823 | 650 | 737 | 1190 | 1940 | 1565 | 2430 | 2170 | 2300 |
| 24 | 400 | 106 | 253 | 232 | 620 | 426 | 532 | NS* | |

NS* = no sample available.

TABLE 14C
Pharmacokinetic Profile at Day 1 in Dog PK study #3.

| | 5 mg/kg dose | | | 10 mg/kg dose | | | 20 mg/kg dose | | |
|---|---|---|---|---|---|---|---|---|---|
| PK | Animal | Animal | | Animal | Animal | | Animal | Animal | |
| Parameter | 1 | 2 | Mean | 1 | 2 | Mean | 1 | 2 | Mean |
| Rsq_adj | ND | 1.00 | — | 0.998 | 1.00 | — | 0.990 | 0.998 | — |
| No. points | 0.00 | 3.00 | — | 3.00 | 3.00 | 3.00 | 3.00 | 3.00 | 3.00 |
| used for | | | | | | | | | |
| T½ | | | | | | | | | |
| Cmax | 785 | 686 | 736 | 2010 | 1450 | 1730 | 4330 | 6000 | 5165 |
| (ng/ml) | | | | | | | | | |
| Tmax (h) | 8.00 | 4.00 | 6.00 | 4.00 | 2.00 | 3.00 | 4.00 | 4.00 | 4.00 |
| T½ (h) | — | 5.63 | — | 5.24 | 5.95 | 5.60 | 5.64 | 11.7 | 8.67 |
| Tlast (h) | 24.0 | 24.0 | 24.0 | 24.0 | 24.0 | 24.0 | 24.0 | 24.0 | 24.0 |
| AUC0-last | 11592 | 7398 | 9495 | 19485 | 15568 | 17527 | 49400 | 86304 | 67852 |
| (ng · h/mL) | | | | | | | | | |
| AUC0-inf | — | 7875 | — | 20527 | 16719 | 18623 | 52608 | 116806 | 84707 |
| (ng · h/mL) | | | | | | | | | |
| MRT0-last | 9.77 | 7.83 | 8.80 | 7.59 | 7.84 | 7.72 | 8.19 | 10.1 | 9.15 |
| (h) | | | | | | | | | |
| MRT0-inf | — | 9.30 | — | 8.80 | 9.54 | 9.17 | 9.65 | 18.1 | 13.9 |
| (h) | | | | | | | | | |
| AUCExtra | — | 6.05 | — | 5.08 | 6.88 | 5.98 | 6.10 | 26.1 | 16.1 |
| (%) | | | | | | | | | |
| AUMCExtra | — | 20.9 | — | 18.2 | 23.5 | 20.9 | 20.3 | 58.8 | 39.6 |
| (%) | | | | | | | | | |
| Cl_F | — | 10.6 | — | 8.12 | 9.97 | 9.04 | 6.34 | 2.85 | 4.59 |
| (mL/min/kg) | | | | | | | | | |
| Vz_F (L/kg) | — | 5.16 | — | 3.68 | 5.14 | 4.41 | 3.09 | 2.89 | 2.99 |

TABLE 14D
Pharmacokinetic Profile at Day 5 in Dog PK study #3.

| | 5 mg/kg dose | | | 10 mg/kg dose | | | 20 mg/kg dose | | |
|---|---|---|---|---|---|---|---|---|---|
| PK | Animal | Animal | | Animal | Animal | | Animal | Animal | |
| Parameter | 1 | 2 | Mean | 1 | 2 | Mean | 1 | 2 | Mean |
| Rsq_adj | 0.959 | 0.993 | — | 0.984 | 0.991 | — | 0.994 | -0.989 | — |
| No. points | 3.00 | 3.00 | 3.00 | 3.00 | 3.00 | 3.00 | 3.00 | 3.00 | 3.00 |
| used for | | | | | | | | | |
| T½ | | | | | | | | | |
| Cmax | 1270 | 1040 | 1155 | 1740 | 2780 | 2260 | 3510 | 2220 | 2865 |
| (ng/ml) | | | | | | | | | |
| Tmax (h) | 2.00 | 2.00 | 2.00 | 2.00 | 2.00 | 2.00 | 2.00 | 2.00 | 2.00 |
| T½ (h) | 13.5 | 6.42 | 9.96 | 7.30 | 10.3 | 8.80 | 7.64 | 661* | 334 |
| Tlast (h) | 24.0 | 24.0 | 24.0 | 24.0 | 24.0 | 24.0 | 24.0 | 8.00 | ND |
| AUC0-last | 17790 | 11297 | 14544 | 20531 | 36083 | 28307 | 43537 | 16801 | 30169 |
| (ng · h/mL) | | | | | | | | | |
| AUC0-inf | 25554 | 12279 | 18917 | 22976 | 45289 | 34133 | 49399 | 2087500 | 1068450 |
| (ng · h/mL) | | | | | | | | | |
| MRT0-last | 9.78 | 8.08 | 8.93 | 8.50 | 9.39 | 8.95 | 8.57 | 4.03 | 6.30 |
| (h) | | | | | | | | | |
| MRT0-inf | 20.0 | 10.1 | 15.1 | 11.3 | 15.4 | 13.4 | 11.7 | 955 | 483 |
| (h) | | | | | | | | | |
| AUCExtra | 30.4 | 8.00 | 19.2 | 10.6 | 20.3 | 15.5 | 11.9 | 99.2 | 55.6 |
| (%) | | | | | | | | | |
| AUMCExtra | 65.9 | 26.4 | 46.2 | 32.6 | 51.3 | 42.0 | 35.5 | 100 | 67.8 |
| (%) | | | | | | | | | |
| Cl_F | 3.26 | 6.79 | 5.02 | 7.25 | 3.68 | 5.47 | 6.75 | 0.160 | 3.45 |
| (mL/min/kg) | | | | | | | | | |
| Vz_F (L/kg) | 3.82 | 3.77 | 3.79 | 4.58 | 3.27 | 3.92 | 4.46 | 9.29 | 6.87 |

*The adjusted linear regression coefficient of the concentration value on the terminal phase was less than 0.9, and the T½ therefore might not be accurately calculated.
ND = Not determined (Parameters not determined due to inadequately defined terminal elimination phase).
BQL = Below the lower limit of quantitation (LLOQ). If the adjusted rsq (linear regression coefficient of the concentration value on the terminal phase) is less than 0.9, T½ might not be accurately estimated. If the % AUCExtra > 20%, AUC0-inf, Cl, MRT0-inf and Vdss might not be accurately estimated. If the % AUMCExtra > 20%, MRT0-inf and Vdss might not be accurately estimated.

TABLE 14E
Dose proportionality of Compound A in Dog PK study #3 After
Single PO Administration Day 1.

| | | | | | | | AUC0- |
|---|---|---|---|---|---|---|---|
| | | | Dose | Cmax | Cmax | AUC0-last | last |
| Compared Doses (mg/kg) | | | Ratio | Values | Ratio | Values | Ratio |
| 10.0 | over | 5.00 | 2.00 | 1730/736 | 2.35 | 17527/9495 | 1.85 |
| 20.0 | over | 10.0 | 2.00 | 5165/1730 | 2.99 | 67852/17527 | 3.87 |
| 20.0 | over | 5.00 | 4.00 | 5165/736 | 7.02 | 67852/9495 | 7.15 |

TABLE 14F
Accumulative Index of Compound A in Dogs Following
PO Administration for Dog PK study #3.

| | Cmax | Accumulative | | Accumulative |
|---|---|---|---|---|
| Dose | (Day 5 | Index of Cmax | AUC0-last | Index of |
| Level | and | (Day 5/ | (Day 5 and | AUC0-last |
| (mg/kg) | Day 1) | Day 1) | Day 1) | (Day 5/Day 1) |
| 5.00 | 1155/736 | 1.57 | 14544/9495 | 1.53 |
| 10.0 | 2260/1730 | 1.31 | 28307/17527 | 1.62 |
| 20.0 | 2865/5165 | 0.555 | 30169/67852 | 0.445 |

TABLE 14G
Body Weight and Clinical Observations for Dog PK study #3.

| | | Animal | | |
|---|---|---|---|---|
| | | Body | | Clinical |
| Day | Group | Weight (kg) | Mean | Observation |
| 1 | 1 | 8.38 | 8.63 | None |
| | | 8.88 | | None |
| | 2 | 9.37 | 8.87 | None |
| | | 8.36 | | None |
| | 3 | 9.24 | 9.02 | None |
| | | 8.79 | | None |
| 2 | 1 | 8.44 | 8.89 | None |
| | | 9.33 | | None |
| | 2 | 9.63 | 9.14 | None |
| | | 8.65 | | None |
| | 3 | 9.44 | 9.35 | None |
| | | 9.25 | | None |
| 3 | 1 | 8.45 | 8.87 | None |
| | | 9.29 | | None |
| | 2 | 9.5 | 9.10 | None |
| | | 8.69 | | None |
| | 3 | 9.32 | 9.14 | None |
| | | 8.96 | | None |
| 4 | 1 | 8.45 | 8.87 | None |
| | | 9.29 | | None |
| | 2 | 9.43 | 9.02 | None |
| | | 8.6 | | None |
| | 3 | 9.13 | 8.87 | None |
| | | 8.6 | | None |
| 5 | 1 | 8.22 | 8.70 | None |
| | | 9.18 | | None |
| | 2 | 9.31 | 8.87 | None |
| | | 8.43 | | None |
| | 3 | 8.88 | 8.50 | None |
| | | 8.11 | | On day 6 |
| | | | | this animal |
| | | | | was found |
| | | | | dead |

Notably, Compound A plasma concentrations for all doses administered remained above the free EC50 BD2 for 24 hours, and all white blood cell fraction parameters were within range (see Table 15A). Without being bound by any particular theory, there was no evidence of a dose-dependent effect on hematology parameters, and Compound A exhibited a large therapeutic window. FIG. 7L shows the plasma concentration of Compound A over 24 hours at three different doses (1 mg/kg (IV), 5 mg/kg (PO), and 10 mg/kg (PO)) relative to the free EC50 for BD1 and the free EC50 for BD2, which had been previously determined. The data points for the PO 5 mg/kg and 10 mg/kg doses and for the IV 1 mg/kg dose are well within the broad window between the free EC50 for BD1 and the free EC50 for BD2.

As can be seen from Table 15A, the hematology parameters at 5 mg/kg/day and at 10 mg/kg/day were within normal ranges. Even at the highest dose of 20 mg kg/day the hematology parameters (not shown) in the one animal measured were within normal ranges.

TABLE 15A
Effect of Compound A on Hematology Parameters
(Day 5 Data) for Dog PK study #3.

| | | 5 mg/kg/day | 10 mg/kg/day |
|---|---|---|---|
| Parameter | Range | (mean) | (mean) |
| White Blood Cells, | 4.81-15.67 | 10.59 | 10.86 |
| 103/μL | | | |
| Neutrophils, 10³/μL | 1.80-10.83 | 7.08 | 6.59 |
| Lymphocytes, 10³/μL | 1.42-3.76 | 2.72 | 3.31 |
| Monocytes, 10³/μL | 0.11-1.08 | 0.31 | 0.48 |
| Eosinophils, 10³/μL | 0.00-1.24 | 0.38 | 0.40 |
| Basophils, 10³/μL | 0.00-0.19 | 0.08 | 0.07 |
| Platelets, 10³/μL | 127-393 | 224.0 | 273.0 |

### Dog PK Study #4

The toxicokinetics of Compound A was assessed in beagle dogs over a 14-day period in Dog PK study #4, as described in Experimental Method B. The plasma concentration of Compound A at three different doses (1 mg/kg, 3 mg/kg, and 10 mg/kg) was recorded on Day 14, following 14 days of daily administration of Compound A (Table 15B). FIG. 7M shows the mean plasma concentration for each tested dose (1 mg/kg, 3 mg/kg, and 10 mg/kg) relative to the free EC50 for BD1 BRD4 and BD2 BRD4. As shown in FIG. 7M, Compound A plasma concentrations following 14 days of administration remained well above the BD2 BRD4 EC50 and far below the BD1 BRD EC50 for all tested time points and applied doses over the 24-hour testing period and demonstrated a dose-dependent increase in plasma concentration. Further, there was no exposure differences in Compound A plasma concentrations between male and female animals. For all animals, plasma concentration of Compound A peaked (or plateaued out) at approximately 2 hours (i.e., some point between the 1 hour and 4 hour time points) after administration of Compound A. Table 15C further illustrates the impact of Compound A on platelet counts, as measured on Day 15, following 14 days of daily administration of Compound A (1 mg/kg, 3 mg/kg, and 10 mg/kg). There was no appreciable difference in platelet counts in animals receiving any dose of Compound A relative to those receiving vehicle (0 mg/kg data point). Thus, the data show that there is no evidence of Compound A reducing platelet counts, or of reduced platelet counts in animals receiving administration of Compound A.

TABLE 15B
Mean Compound A Plasma Concentration after Daily Administration for 14 Days.

| | | Mean Plasma Concentration (ng/mL) | | | | | |
|---|---|---|---|---|---|---|---|
| Dose | Group | 0.5 h | 1 h | 2 h | 4 h | 8 h | 24 h |
| 1 mg/kg | Male | 31.6 | 56.7 | 60.8 | 44.8 | 24.5 | 25.1 |
| | Female | 27.8 | 57.9 | 70.1 | 55.9 | 25.7 | 10.3 |
| | Pooled Mean | 29.7 | 57.3 | 65.45 | 50.35 | 25.1 | 17.7 |
| 3 mg/kg | Male | 98.3 | 201 | 265 | 245 | 165 | 33 |
| | Female | 152 | 324 | 320 | 230 | 87.5 | 14.2 |
| | Pooled Mean | 125.15 | 262.5 | 292.5 | 237.5 | 126.25 | 23.6 |
| 10 mg/kg | Male | 1010 | 1450 | 1490 | 1350 | 646 | 145 |
| | Female | 1130 | 1630 | 1680 | 1070 | 342 | 61.6 |
| | Pooled Mean | 1070 | 1540 | 1585 | 1210 | 494 | 103.3 |

*The free EC50 BD1 BRD4 was 64722 ng/ml, and the free EC50 BD2 BRD4 was 6.73 ng/mL.

TABLE 15C
Mean Platelet Counts on Day 15 following
14 Days of Compound A Administration.

| Dose | Mean Count (×10³/μL) |
|---|---|
| 0 mg/kg (Vehicle) | 249.75 |
| 1 mg/kg (Compound A) | 260.75 |
| 3 mg/kg (Compound A) | 241 |
| 10 mg/kg (Compound A) | 293 |

*The maximum platelet count was 442 (×10³/μL) and the minimum platelet count was 128 (×10³/μL).

TABLE 15D
Outline of the Four Dog pK Studies.

| | | | | Dose |
|---|---|---|---|---|
| Study | Type | Formulation | Route | (mg/kg) |
| 1 | Beagle | 20% propylene glycol/20% | IV | 1 |
| | | Vitamin E TPGS/60% water | | |
| | Beagle | 20% propylene glycol/20% | PO | 5 |
| | | Vitamin E TPGS/60% water | | |
| 2 | Beagle | HPβCD (20% solution | IV | 1 |
| | | w/v)/DMSO (99/1) | | |
| 3 | Beagle | 5% DMSO/15% PEG-400/80% | PO | 5 |
| | | (10% VE-TPGS) | | |
| | Beagle | 5% DMSO/15% PEG-400/80% | PO | 10 |
| | | (10% VE-TPGS) | | |
| | Beagle | 5% DMSO/15% PEG-400/80% | PO | 20 |
| | | (10% VE-TPGS) | | |
| 4 | Beagle | 5% DMSO/15% PEG-400/80% | PO | 1 |
| | | (10% VE-TPGS) | | |
| | Beagle | 5% DMSO/15% PEG-400/80% | PO | 3 |
| | | (10% VE-TPGS) | | |
| | Beagle | 5% DMSO/15% PEG-400/80% | PO | 10 |
| | | (10% VE-TPGS) | | |

### In Vitro Characterization of Compound A

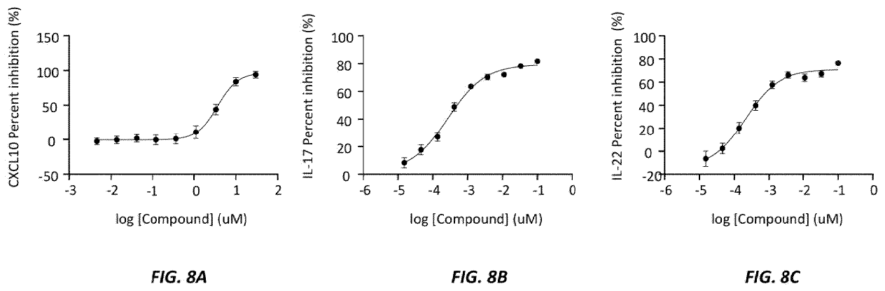

The selectivity of Compound A in vitro against Th17 cytokines was additionally assessed. As measured by ELISA, Compound A exhibited high inhibitory potency against IL-17A (FIG. 8B) and IL-22 (FIG. 8C), with minimal inhibition of CXCL10 (FIG. 8A) (Table 16A). The Hill Slope of the IC50 for IL-17 was 0.819, the Hill Slope of the IC50 for IL-22 was 0.894, and the Hill Slope for the IC50 for CXCL10 was 1.778. Specifically, Compound A inhibited the release of IL-17A and IL-22 at a sub-nanomolar IC50 (see Table 16A). Compound A was about 13,000-fold more potent against inhibiting IL-17A than CXCL10, and about 17,000-fold more potent in inhibiting IL-22 than CXCL10. Compound A's IC50 for IL-17A was also more potent that the reported IC50 for IL-17A for both secukinumab (0.37 nM; Cosentyx-Novartis) and DC-806 (5.7 nM; Dice Therapeutics). Compound A's IC50 for IL-17A was lower than the reported IC50 for IL-17A for Secukinumab (0.37 nM; Cosentyx-Novartis; Summary basis of approval for Cosentyx, pharmacology review, CDER, FDA, August 2014) as well as the IL50 for IL-17A for DC-806 (5.7 nM; Dice Therapeutics; Dice Therapeutics Corporate Presentation March 2023). Compound A inhibition of IL-17A, IL-22, and CXCL10 are also depicted showing the percent inhibition relative to control in Tables 16B-16D. Tables 16E-16F depict preliminary results from the biomarker assays, and the results from the preliminary analyses (Tables 16E-16F) are included in the data presented in Tables 16A-16D. Tables 16E and 16F additionally show that Compound A was seen to be more effective than GSK620. In addition to showing a superior activity relative to GSK620 in reducing the inflammatory biomarkers IL-17A, II-22 and CXCL10, Compound A was more selective than GSK620 for BDII by greater than about two orders of magnitude.

TABLE 16A
Selectivity of Compound A for inhibiting Th17 cytokines.

Selectivity (IC50)

| | CXCL10 | IL-17A | IL-22 |
|---|---|---|---|
| Compound A | 3637 nM | 0.271 nM | 0.214 nM |

TABLE 16B
Compound A Inhibition of IL-17A.

| Compound | Percent Inhibition Relative to Control | | | | | | | | | | | | | |
|---|---|---|---|---|---|---|---|---|---|---|---|---|---|---|
| Conc. (μM) | Donor 1 | | Donor 2 | | Donor 3 | | Donor 4 | | Donor 5 | | Donor 6 | | Donor 7 | |
| 0.1 | 80.77 | 80.42 | 83.72 | 86.56 | 80.96 | 85.39 | 77.87 | 84.04 | 85 | 80.29 | 80.05 | 78.2 | 79.86 | 80.34 |
| 0.033333333 | 76.69 | 78.00 | 81.52 | 83.08 | 69.43 | 73.08 | 77.7 | 78.81 | 89.99 | 83.98 | 75.68 | 76.46 | 73.03 | 78.96 |
| 0.011111111 | 75.17 | 58.72 | 76.44 | 74.19 | 71.49 | 77.11 | 71.75 | 72.53 | 79.11 | 74.77 | 72.8 | 71.45 | 66.86 | 67.16 |
| 0.003703704 | 70.33 | 63.45 | 71.06 | 74.87 | 79.08 | 75.92 | 67.69 | 54.36 | 79.88 | 80.41 | 67.79 | 60.5 | 70.45 | 69.67 |
| 0.001234568 | 56.92 | 62.80 | 67.54 | 70.18 | 58.60 | 66.13 | 59.19 | 60.91 | 75.98 | 75.17 | 53.28 | 57.9 | 62.54 | 63.56 |
| 0.000411523 | 42.92 | 60.62 | 51.01 | 51.11 | 62.34 | 51.68 | 47.46 | 28.29 | 56.37 | 60.88 | 49.48 | 23.24 | 46.54 | 52.23 |
| 0.000137174 | 30.76 | 4.46 | 42.02 | 55.02* | 40.50 | 25.91 | 19.06 | 25.23 | 32.91 | 39.93 | 12.53 | 17.16 | 22.57 | 38.63 |
| 4.57247E−05 | −1.00 | −0.28 | 32.68 | 27.06 | 28.89 | 20.58 | 2.83 | 7.11 | 42.94 | 28.73 | 7.45 | 18.97 | 13.64 | 16.93 |
| 1.52416E−05 | −2.11 | −11.47 | 18.94 | 28.57 | 13.94 | 37.95 | −8.34 | 0.61 | 18.05 | 5.95 | 4.5 | 4.36 | −0.57 | 6.2 |

*Data point was excluded from analysis based on experimental error.

TABLE 16C
Compound A Inhibition of IL-22.

| Compound | Percent Inhibition Relative to Control | | | | | | | | | | | |
|---|---|---|---|---|---|---|---|---|---|---|---|---|
| Conc. (μM) | Donor 1 | | Donor 2 | | Donor 3 | | Donor 4 | | Donor 5 | | Donor 6 | |
| 0.1 | 72.76 | 67.92 | 74.39 | 71.46 | 84.71 | 87.61 | 80.76 | 81.88 | 60.98 | 68.86 | 83.13 | 82.57 |
| 0.033333333 | 59.22 | 66.02 | 52.09 | 58.24 | 79.61 | 59.59 | 77.99 | 81.27 | 61.98 | 62.46 | 72.37 | 75.81 |
| 0.011111111 | 52.73 | 48.74 | 54.26 | 71.04 | 74.68 | 76.83 | 75.01 | 72.99 | 61.33 | 45.71 | 72.33 | 61.13 |
| 0.003703704 | 71.96 | 68.04 | 61.67 | 51.81 | 80.73 | 79.65 | 75.51 | 62.59 | 54.19 | 57.94 | 67.89 | 62.44 |
| 0.001234568 | 58.12 | 57.50 | 65.37 | 75.65 | 47.36 | 70.12 | 62.59 | 69.46 | 42.31 | 42.53 | 46.04 | 55.94 |
| 0.000411523 | 27.24 | 56.28 | 11.33* | 49.71 | 35.72 | 28.88 | 47.8 | 62.34 | 36.26 | 13.07 | 35.55 | 45.22 |
| 0.000137174 | 36.49 | 13.22 | 40.62 | 42.44 | −18.99 | 2.10 | 20.38 | 35.83 | 13.68 | 20.77 | 20.31 | 15 |
| 4.57247E−05 | −1.30 | −19.37 | 12.73 | 33.63 | −8.80 | 13.17 | 6.1 | 16.75 | −8.17 | −19.44 | 7.09 | −4.72 |
| 1.52416E−05 | −4.73 | −5.22 | 11.96 | 21.53 | −30.35 | −52.69 | −14.1* | −16.32* | −0.25 | 3.62 | −2.47 | −6.99 |

*Data point was excluded from analysis based on experimental error.

TABLE 16D
Compound A Inhibition of CXCL10.

| Compound | | Percent Inhibition Relative to Control | | | | | | | | | | | |
|---|---|---|---|---|---|---|---|---|---|---|---|---|---|
| Conc. (μM) | | Donor 1 | | Donor 2 | | Donor 3 | | Donor 4 | | Donor 5 | | Donor 6 | |
| 30 | | 122.94 | 124.38 | 87.54 | 80.12 | 123.10 | 123.75 | 58.43 | 68.15 | 37.89* | 48.38* | 13.06* | −3.36* |
| 10 | | 117.19 | 119.13 | 64.26 | 65.60 | 118.54 | 120.49 | 84.37 | 87.96 | 65.18 | 69.04 | 31.06 | 23.69 |
| 3.333333333 | | 78.69 | 79.69 | 21.63 | 4.53 | 76.57 | 87.28 | 38.54 | 40.34 | 20.00 | 27.90 | −14.27 | −10.07 |
| 1.111111111 | | 27.06 | 41.25 | −27.6* | −38.41* | 31.62 | 48.53 | 9.08 | 15.14 | −12.57 | −9.97 | −18.19 | −43.93 |
| 0.37037037 | | 35.44 | 35.81 | −6.18 | −25.95 | 17.81 | 17.87 | 3.10 | −0.26 | −10.31 | −8.12 | −43.09 | −37.86 |
| 0.12345679 | | 4.44 | 21.56 | 3.91 | −20.29 | 9.82 | 8.52 | −1.91 | −4.52 | −6.44 | −5.10 | −29.66 | −55.02 |
| 0.041152263 | | 17.38 | 18.25 | 4.12 | −7.31 | 9.34 | 0.69 | −1.46 | 5.12 | 0.19 | 3.80 | −12.68 | −18.09 |
| 0.013717421 | | 20.44 | 19.00 | 3.91 | −4.33 | −7.30 | −17.68 | −12.82 | −1.46 | 2.29 | 9.34 | −11.66 | −6.71 |
| 0.004572474 | | −1.63 | 4.06 | −3.50 | −2.16 | −6.70 | 1.07 | −11.25 | −6.84 | 3.46 | −1.41 | −21.26 | −23.97 |
| | Compound | Percent Inhibition Relative to Control | | | | | | | | | | | |
| | Conc. (μM) | Donor 7 | | | Donor 8 | | | Donor 9 | | | Donor 10 | | |
| | 30 | 94.95 | 94.14 | 99.8 | 85.5 | 79.52 | 85.21 | 115.03 | 115.53 | 115.96 | 60.64 | 72.98 | 75.24 |
| | 10 | 101.41 | 101.41 | 100.61 | 86.09 | 86.24 | 85.65 | 112.61 | 113.18 | 113.47 | 38.19 | 51.53 | 56.31 |
| | 3.333333333 | 73.13 | 66.67 | 61.01 | 44.05 | 40.36 | 52.92 | 89.74 | 91.02 | 96.94 | −29.68 | 17.91 | −0.56 |
| | 1.111111111 | 22.22 | 18.18 | 5.25 | 28.39 | 33.64 | 36.89 | 64.94 | 72.35 | 79.84 | −76.63 | −72.22 | −56.35 |
| | 0.37037037 | −3.64 | −5.25 | −18.99 | 22.92 | 28.83 | 35.63 | 44.35 | 57.68 | 63.8 | −63.11 | −51.4 | 60.59 |
| | 0.12345679 | −27.07 | −24.65 | −27.88 | 24.03 | 37.4 | 36.96 | 41.15 | 64.3 | 73.57 | −45.9 | −41.3 | −41.48 |
| | 0.041152263 | −21.41 | −27.88 | −32.73 | 8.51 | 13.76 | 36.07 | 35.02 | 50.84 | 63.88 | −26.61 | −33.01 | −40.49 |
| | 0.013717421 | −17.37 | −31.92 | −59.39 | 0.54 | 6.52 | 33.19 | 26.54 | 47.42 | 67.01 | −17.51 | −19.76 | −38.24 |
| | 0.004572474 | −13.33 | −33.54 | −45.66 | 0.98 | 19.97 | 28.68 | 23.48 | 44.21 | 56.96 | −16.79 | −23.73 | −29.04 |

*Data point was excluded from analysis based on experimental error.

TABLE 16E
Preliminary biomarker assay

| | IL-17A IC50 (nM) | | | IL-22 IC50 (nM) | | | CXCL 10 IC50 (nM) | | |
|---|---|---|---|---|---|---|---|---|---|
| | Test | Test | Test | Test | Test | Test | Test | Test | Test |
| | Subject | Subject | Subject | Subject | Subject | Subject | Subject | Subject | Subject |
| Compound | 1 | 2 | 3 | 1 | 2 | 3 | 1 | 2 | 3 |
| GSK620 | 80.5 | 59.7 | 139.3 | 231.3 | 21.6 | 106.1 | >30000 | >30000 | >30000 |
| Compound A | 0.2 | 0.3 | 0.4 | 0.2 | 0.1 | 0.3 | 1001 | — | — |

TABLE 16F
Average preliminary biomarker assay

| | | | | | | | Biomarker | | Biomarker | |
|---|---|---|---|---|---|---|---|---|---|---|
| | IL-17A IC50 | | IL-22 IC50 | | CXCL10 IC50 | | selectivity (IL-17 | | selectivity (IL-22 | |
| | (nM) | | (nM) | | (nM) | | vs. CXCL10) | | vs. CXCL 10) | |
| Compound | Mean | SEM | Mean | SEM | Mean | SEM | Modifier | Fold | Modifier | Fold |
| GSK620 | 93.2 | 23.8 | 119.7 | 60.9 | >30000 | | | 322 | | 251 |
| Compound A | 0.3 | | 0.2 | | 1001.0 | | | 3492 | | 5178 |

Without being bound by any particular theory, the data suggest that selective inhibition of the Th17 cytokines can be achieved by administration of Compound A. Thus, Compound A may be an effective treatment for diseases that are specifically mediated and/or exacerbated through a Th17 response (e.g., psoriasis, rheumatoid arthritis, and renal fibrosis). Without being bound by any particular theory, whereas other therapeutics target the Th17 cytokines through inhibition of extracellular cytokines, inhibition of cytokine receptors, or inhibition of cytokine receptor signal transduction, BET inhibitors (Compound A) disrupt transcription of inflammatory gene transcription in T cells, thereby circumventing the complex external signaling pathways in immuno- and fibro-inflammatory diseases.

When compared to other BD-2 selective BET inhibitors in development (based on readily available public information, such as clinicaltrials.gov, academic publications, and corporate websites and presentations) and/or that have been developed, Compound A exhibits a lower EC50, a higher selectivity, and greater oral bioavailability than NUV-868 (Nuvation), ABBV-744 (AbbVie), GSK620 (GSK), Pelabresib (Constellation), ABBV-075 (AbbVie), MK-8628/OTX-015, and BI-894999 (see Table 15G).

TABLE 16G
Characterization of Compound A, Compound B, Compound C, and
Compound D Relative to Other BD2-Selective BET Inhibitors.

| | | Selectivity** |
|---|---|---|
| Compound Name | BD2* (nM) | (BD1/BD2) |
| Compound A | < | >>>> |
| Compound B | ≤ | >>>> |
| Compound C | < | >>>> |
| Compound D | < | >>>> |
| NUV-868² | 2 | 1,460x (FRET) |
| ABBV-744³ | 28 | 753x (FRET) |
| GSK620⁴ | 79 | 220x |
| Pelabresib² | 17 | 5x (FRET) |
| ABBV-075³ | 13 | 2.6x |
| MK-8628/OTX-015⁵ | 26 | 1.5x |
| BI-894999⁶ | 41 | 0.1x |

*Lower number denotes higher potency
**Higher number denotes higher selectivity. Data based on nanoBRET assay unless otherwise indicated.
**Not a head-to-head trial
²Nuvation corporation presentation (May 2023);
³Faivre et al., Nature, 578: 306-310 (2020);
⁴Delmont et al., J. Med. Chem., 63(17): 9093-9126 (2020);
⁵Wang et al., J. Med. Chem., 58(12): 4927-39 (2017);
⁶Kraut et al., Oncogene, 37: 2687-2701 (2018).

### Example 118: Treating Psoriasis with a BET Inhibitor and Vehicle

The imiquimod-induced model of psoriasis was used to assess the efficacy of Compound A in treating psoriasis. Evaluation of the different doses of Compound A compared to vehicle and controls in imiquimod-induced psoriasis in mice was undertaken in accordance with the protocol set out in Experimental Method C. Treatment started on Day 8 after induction so reference to day 1 of treatment is to Day 8 and day 7 of treatment is to Day 14.

It was initially noted that only in the deucravacitinib treatment group two animals died during the study. The first animal died after the third treatment, and the other animal died after the fourth treatment. For both animals, no abnormalities were observed at autopsy.

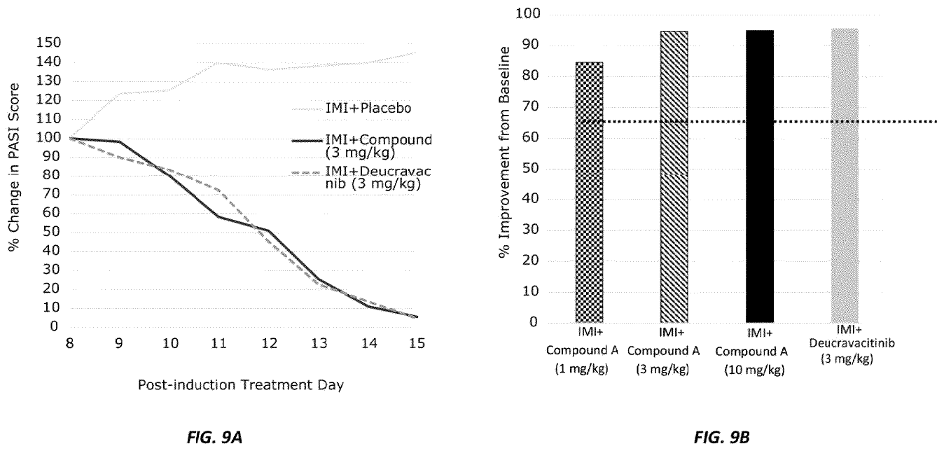

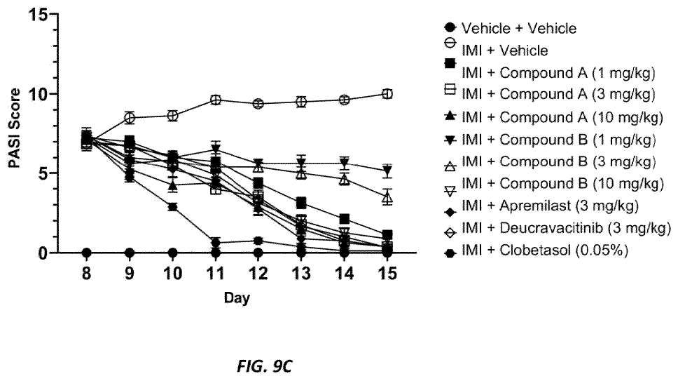

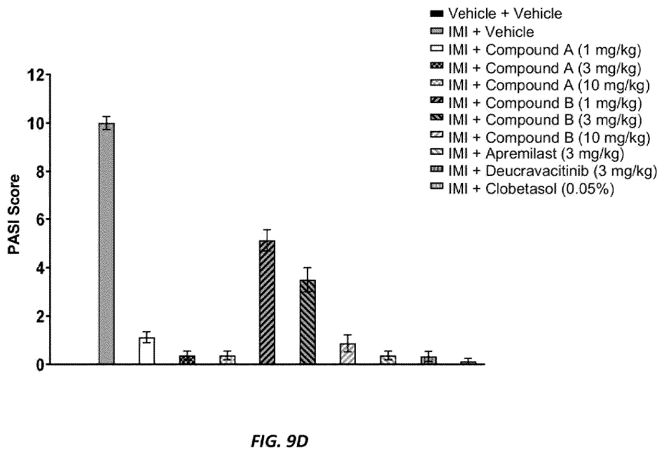

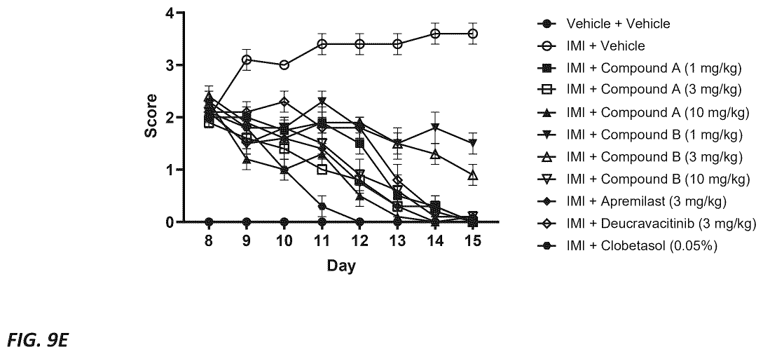

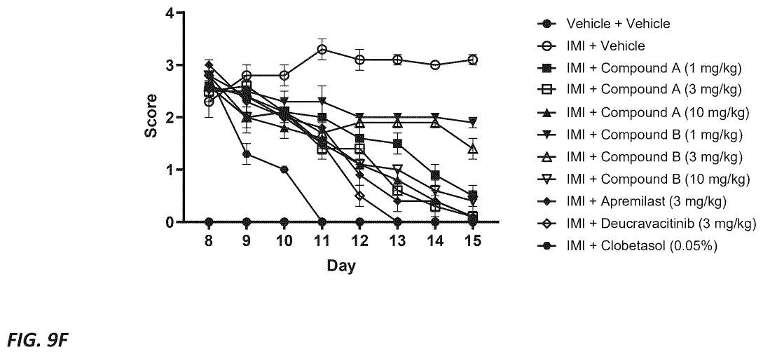

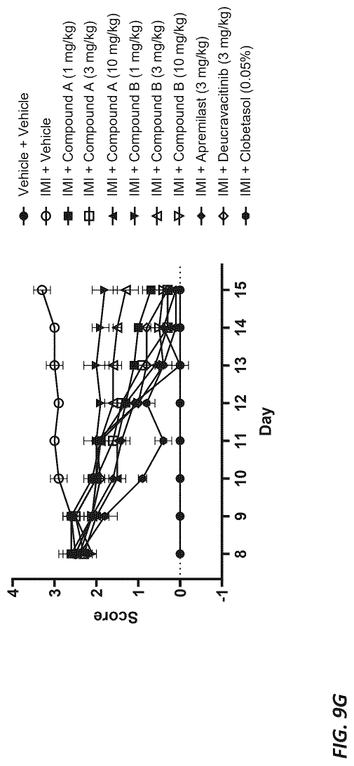

The efficacy of Compound A was compared against Deucravacitinib, an FDA-approved drug for plaque psoriasis. Treatment efficacy was assessed using an in vivo model of psoriasis, where psoriasis is induced in dorsal depilated mice by topical imiquimod cream. As shown in FIG. 9A, comparable doses of Compound A and deucravacitinib both surpassed PASI-90 (where the change in the PASI score is 90%) by day 7 of treatment. FIG. 9A further shows that the onset of activity for both deucravacitinib and Compound A (3 mg/kg) were similar. Notably, starting on day 9 (after one day of treatment) and continuing throughout the remainder of the study, all treatment groups exhibited significantly lower PASI scores relative to animals in the IMI+Vehicle group. Treatment with 1 mg/kg of Compound A resulted in a PASI score improvement of >80% by day 15, and both 3 mg/kg and 10 mg/kg resulted in an improvement in PASI scores of >90% for animals, comparable to the improvement achieved when treating animals with deucravacitinib (FIG. 9B). FIG. 9C and Table 17A further depict the changes in PASI scores for all animals and mean changes (Table 17B) over the course of the study. It is notable that all animals receiving Compound A or Compound B exhibited a decrease in overall score at day 15 relative to day 8. Further, animals receiving Compound A (3 mg/kg) surpassed PASI-90 by day 7 of treatment. FIG. 9D depicts mean PASI scores for all animals on day 15. A dose-dependent decrease in PASI scores was noted for animals being treated with Compound B, with the 10 mg/kg dose proving the most effective at reducing the PASI score. It is notable that PASI scores for animals treated with Compound A (all doses), were graded at a score similar to animals treated with apremilast, deucravacitinib, or clobetasol.

TABLE 17A
PASI Scores in IMI-Psoriasis Model.

| | Animal | | | | | | | | |
|---|---|---|---|---|---|---|---|---|---|
| Group | Number# | D8 | D9 | D10 | D11 | D12 | D13 | D14 | D15 |
| 1 (Vehicle + Vehicle) | 1 | 0 | 0 | 0 | 0 | 0 | 0 | 0 | 0 |
| | 2 | 0 | 0 | 0 | 0 | 0 | 0 | 0 | 0 |
| | 3 | 0 | 0 | 0 | 0 | 0 | 0 | 0 | 0 |
| | 4 | 0 | 0 | 0 | 0 | 0 | 0 | 0 | 0 |
| | 5 | 0 | 10 | 0 | 0 | 0 | 0 | 0 | 0 |
| | 6 | 0 | 0 | 0 | 0 | 0 | 0 | 0 | 0 |
| | 7 | 0 | 10 | 0 | 0 | 0 | 0 | 0 | 0 |
| | 8 | 0 | 0 | 0 | 0 | 0 | 0 | 0 | 0 |
| 2 (IMI + Vehicle) | 1 | 8 | 7 | 8 | 10 | 9 | 9 | 9 | 9 |
| | 2 | 7 | 9 | 9 | 10 | 9 | 9 | 10 | 11 |
| | 3 | 6 | 10 | 9 | 11 | 10 | 9 | 10 | 10 |
| | 4 | 7 | 8 | 8 | 9 | 9 | 8 | 9 | 9 |
| | 5 | 8 | 8 | 7 | 9 | 9 | 10 | 10 | 10 |
| | 6 | 7 | 8 | 10 | 9 | 9 | 10 | 9 | 11 |
| | 7 | 6 | 8 | 9 | 10 | 10 | 10 | 10 | 10 |
| | 8 | 6 | 10 | 9 | 9 | 10 | 11 | 10 | 10 |
| 3 (IMI + Compound A 1 | 1 | 8 | 7 | 7 | 6 | 6 | 3 | 1 | 1 |
| mg/kg) | 2 | 6 | 7 | 6 | 4 | 4 | 2 | 2 | 1 |
| | 3 | 7 | 6 | 5 | 4 | 4 | 2 | 1 | 2 |
| | 4 | 8 | 9 | 6 | 5 | 4 | 3 | 3 | 1 |
| | 5 | 6 | 7 | 5 | 8 | 4 | 4 | 2 | 1 |
| | 6 | 8 | 6 | 6 | 7 | 5 | 3 | 2 | 0 |
| | 7 | 7 | 8 | 5 | 6 | 5 | 5 | 3 | 2 |
| | 8 | 8 | 6 | 8 | 6 | 3 | 3 | 3 | 1 |
| 4 (IMI + Compound A 3 | 1 | 8 | 8 | 6 | 4 | 3 | 2 | 2 | 1 |
| mg/kg) | 2 | 6 | 8 | 7 | 4 | 4 | 2 | 0 | 0 |
| | 3 | 7 | 8 | 4 | 3 | 2 | 2 | 1 | 0 |
| | 4 | 5 | 5 | 5 | 5 | 5 | 0 | 0 | 1 |
| | 5 | 9 | 6 | 5 | 3 | 4 | 3 | 0 | 0 |
| | 6 | 7 | 7 | 6 | 5 | 5 | 2 | 2 | 0 |
| | 7 | 7 | 6 | 6 | 3 | 3 | 2 | 1 | 1 |
| | 8 | 6 | 6 | 5 | 5 | 2 | 1 | 0 | 0 |
| 5 (IMI + Compound A | 1 | 6 | 3 | 2 | 5 | 5 | 2 | 1 | 0 |
| 10 mg/kg) | 2 | 9 | 6 | 4 | 6 | 3 | 1 | 0 | 0 |
| | 3 | 7 | 8 | 4 | 5 | 1 | 2 | 0 | 1 |
| | 4 | 5 | 6 | 5 | 5 | 2 | 2 | 1 | 0 |
| | 5 | 7 | 4 | 3 | 3 | 2 | 1 | 1 | 1 |
| | 6 | 8 | 5 | 5 | 3 | 2 | 1 | 0 | 0 |
| | 7 | 8 | 3 | 5 | 4 | 4 | 2 | 1 | 1 |
| | 8 | 7 | 7 | 6 | 4 | 4 | 1 | 1 | 0 |
| 6 (IMI + Compound B 1 | 1 | 7 | 8 | 6 | 8 | 6 | 4 | 4 | 5 |
| mg/kg) | 2 | 6 | 9 | 5 | 6 | 6 | 5 | 7 | 7 |
| | 3 | 7 | 5 | 5 | 4 | 4 | 4 | 4 | 4 |
| | 4 | 8 | 8 | 7 | 6 | 6 | 5 | 6 | 6 |
| | 5 | 8 | 6 | 6 | 7 | 6 | 8 | 6 | 6 |
| | 6 | 8 | 8 | 7 | 9 | 6 | 7 | 7 | 5 |
| | 7 | 6 | 5 | 6 | 6 | 6 | 5 | 5 | 3 |
| | 8 | 6 | 5 | 6 | 6 | 5 | 7 | 6 | 5 |
| 7 (IMI + Compound B 3 | 1 | 8 | 7 | 6 | 5 | 4 | 6 | 6 | 4 |
| mg/kg) | 2 | 7 | 8 | 8 | 5 | 6 | 4 | 4 | 5 |
| | 3 | 7 | 6 | 5 | 5 | 6 | 4 | 5 | 3 |
| | 4 | 7 | 6 | 4 | 6 | 6 | 4 | 3 | 1 |
| | 5 | 7 | 6 | 6 | 5 | 5 | 6 | 4 | 2 |
| | 6 | 6 | 4 | 6 | 7 | 6 | 6 | 6 | 5 |
| | 7 | 9 | 6 | 6 | 5 | 6 | 6 | 4 | 4 |
| | 8 | 8 | 5 | 5 | 5 | 4 | 4 | 5 | 4 |
| 8 (IMI + Compound B | 1 | 7 | 5 | 6 | 6 | 3 | 1 | 1 | 1 |
| 10 mg/kg) | 2 | 9 | 7 | 4 | 5 | 3 | 1 | 0 | 0 |
| | 3 | 7 | 6 | 4 | 5 | 2 | 1 | 1 | 1 |
| | 4 | 7 | 7 | 8 | 5 | 4 | 2 | 1 | 1 |
| | 5 | 5 | 5 | 8 | 5 | 5 | 4 | 2 | 0 |
| | 6 | 8 | 6 | 6 | 5 | 5 | 3 | 2 | 3 |
| | 7 | 7 | 4 | 6 | 5 | 2 | 2 | 2 | 0 |
| | 8 | 8 | 5 | 5 | 3 | 1 | 2 | 1 | 1 |
| 9 (IMI + Apremilast 3 | 1 | 9 | 8 | 6 | 5 | 3 | 1 | 2 | 1 |
| mg/kg) | 2 | 7 | 5 | 7 | 5 | 5 | 0 | 0 | 0 |
| | 3 | 8 | 5 | 4 | 4 | 3 | 1 | 1 | 0 |
| | 4 | 8 | 7 | 6 | 5 | 2 | 0 | 1 | 0 |
| | 5 | 7 | 6 | 4 | 4 | 3 | 2 | 0 | 1 |
| | 6 | 6 | 4 | 4 | 4 | 3 | 1 | 1 | 0 |
| | 7 | 7 | 6 | 4 | 5 | 1 | 0 | 0 | 0 |
| | 8 | 7 | 6 | 7 | 4 | 2 | 2 | 1 | 1 |
| | 1 | 6 | 7 | 6 | 6 | 4 | 2 | 1 | 1 |
| 10 (IMI + | 2 | 6 | 6 | 6 | — | — | — | — | — |
| Deucravacitinib 3 | 3 | 7 | 7 | 7 | 6 | 3 | 0 | 1 | 0 |
| mg/kg) | 4 | 10 | 8 | 6 | 5 | 4 | 0 | 0 | 0 |
| | 5 | 8 | 6 | 6 | 5 | 3 | 4 | 2 | 1 |
| | 6 | 6 | 7 | 6 | 6 | 3 | 2 | 1 | 0 |
| | 7 | 8 | 6 | 6 | 4 | 3 | 2 | 1 | 0 |
| | 8 | 8 | 6 | — | — | — | — | — | — |
| 11 (IMI + Clobetasol | 1 | 7 | 6 | 3 | 0 | 1 | 1 | 0 | 0 |
| 0.05%) | 2 | 8 | 4 | 2 | 0 | 1 | 1 | 0 | 0 |
| | 3 | 7 | 6 | 2 | 1 | 1 | 0 | 1 | 0 |
| | 4 | 7 | 5 | 3 | 0 | 1 | 0 | 0 | 0 |
| | 5 | 7 | 4 | 3 | 2 | 1 | 0 | 0 | 0 |
| | 6 | 7 | 4 | 3 | 0 | 1 | 0 | 0 | 0 |
| | 7 | 8 | 4 | 3 | 2 | 0 | 1 | 0 | 0 |
| | 8 | 8 | 5 | 4 | 0 | 0 | 0 | 0 | 1 |

#Each animal actually had a different number from 1 to 88, and the animal numbers are shown for simplicity as 1-8 for each group.

TABLE 17B
Mean PASI Scores in IMI-Psoriasis Model

| Group | D8 | D9 | D10 | D11 | D12 | D13 | D14 | D15 |
|---|---|---|---|---|---|---|---|---|
| 1 (Vehicle + | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 |
| Vehicle) | | | | | | | | |
| 2 (IMI + | 6.9 ± 0.3 | 8.5 ± 0.4 | 8.6 ± 0.3 | 9.6 ± 0.3 | 9.4 ± 0.2 | 9.5 ± 0.3 | 9.6 ± 0.2 | 10.0 ± 0.3 |
| Vehicle)* | | | | | | | | |
| 3 (IMI + | 7.3 ± 0.3 | 7.0 ± 0.4 | 6.0 ± 0.4 | 5.8 ± 0.5 | 4.4 ± 0.3 | 3.1 ± 0.4 | 2.1 ± 0.3 | 1.1 ± 0.2 |
| Compound A 1 | | (p = 0.02) | (p = 0.002) | (p < 0.001) | (p < 0.001) | (p < 0.001) | (p < 0.001) | (p < 0.001) |
| mg/kg) | | | | | | | | |
| 4 (IMI + | 6.9 ± 0.4 | 6.8 ± 0.4 | 5.5 ± 0.3 | 4.0 ± 0.3 | 3.5 ± 0.4 | 1.8 ± 0.3 | 0.8 ± 0.3 | 0.4 ± 0.2 |
| Compound A 3 | | (p = 0.015) | (p = 0.001) | (p < 0.001) | (p < 0.001) | (p < 0.001) | (p < 0.001) | (p < 0.001) |
| mg/kg) | | | | | | | | |
| 5 (IMI + | 7.1 ± 0.4 | 5.3 ± 0.6 | 4.3 ± 0.5 | 4.4 ± 0.4 | 2.9 ± 0.5 | 1.5 ± 0.2 | 0.6 ± 0.2 | 0.4 ± 0.2 |
| Compound A 10 | | (p = 0.003) | (p < 0.001) | (p < 0.001) | (p < 0.001) | (p < 0.001) | (p < 0.001) | (p < 0.001) |
| mg/kg) | | | | | | | | |
| 6 (IMI + | 7.0 ± 0.3 | 6.8 ± 0.6 | 6.0 ± 0.3 | 6.5 ± 0.5 | 5.6 ± 0.3 | 5.6 ± 0.5 | 5.6 ± 0.4 | 5.1 ± 0.4 |
| Compound B 1 | | | (p < 0.001) | (p = 0.002) | (p = 0.002) | (p = 0.001) | (p < 0.001) | (p = 0.001) |
| mg/kg) | | | | | | | | |
| 7 (IMI + | 7.4 ± 0.3 | 6.0 ± 0.4 | 5.8 ± 0.4 | 5.4 ± 0.3 | 5.4 ± 0.3 | 5.0 ± 0.4 | 4.6 ± 0.4 | 3.5 ± 0.5 |
| Compound B 3 | | (p = 0.003) | (p = 0.002) | (p < 0.001) | (p < 0.001) | (p < 0.001) | (p < 0.001) | (p < 0.001) |
| mg/kg) | | | | | | | | |
| 8 (IMI + | 7.3 ± 0.4 | 5.6 ± 0.4 | 5.9 ± 0.5 | 4.9 ± 0.3 | 3.1 ± 0.5 | 2.0 ± 0.4 | 1.3 ± 0.3 | 0.9 ± 0.4 |
| Compound B 10 | | (p = 0.001) | (p = 0.003) | (p < 0.001) | (p < 0.001) | (p < 0.001) | (p < 0.001) | (p < 0.001) |
| mg/kg) | | | | | | | | |
| 9 (IMI + | 7.4 ± 0.3 | 5.9 ± 0.4 | 5.3 ± 0.5 | 4.5 ± 0.2 | 2.8 ± 0.4 | 0.9 ± 0.3 | 0.8 ± 0.3 | 0.4 ± 0.2 |
| Apremilast 3 | | (p = 0.003) | (p < 0.001) | (p < 0.001) | (p < 0.001) | (p < 0.001) | (p < 0.001) | (p < 0.001) |
| mg/kg) | | | | | | | | |
| 10 (IMI + | 7.4 ± 0.5 | 6.6 ± 0.3 | 6.1 ± 0.1 | 5.3 ± 0.3 | 3.3 ± 0.2 | 1.7 ± 0.6 | 1.0 ± 0.3 | 0.3 ± 0.2 |
| Deucravacitinib | | (p = 0.003) | (p = 0.001) | (p = 0.002) | (p = 0.002) | (p = 0.002) | (p = 0.002) | (p = 0.001) |
| 3 mg/kg) | | | | | | | | |
| 11 (IMI + | 7.4 ± 0.2 | 4.8 ± 0.3 | 2.8 ± 0.2 | 0.6 ± 0.3 | 0.8 ± 0.2 | 0.4 ± 0.2 | 0.1 ± 0.1 | 0.1 ± 0.1 |
| Clobetasol | | (p < 0.001) | (p < 0.001) | (p < 0.001) | (p < 0.001) | (p < 0.001) | (p < 0.001) | (p < 0.001) |
| 0.05%) | | | | | | | | |

*p values indicate significance of a mean score relative to the IMI + Vehicle score.

PASI score results include the severity of psoriasis as observed and recorded by taking into account the presence of erythema, presence of induration, and peeling of skin. Starting on day 9 (after one day of treatment), animals in the IMI+Vehicle group exhibited significantly higher mean scores for the presence of erythema on the skin at the area of induction of psoriasis than all other treatment groups, and this difference in scores continued for the remainder of the study. On day 9 (after one day of treatment), animals in the IMI+Vehicle group also exhibited significantly higher scores for the presence of induration on the skin at the area of induction of psoriasis relative to animals receiving Compound A (10 mg/kg) and Compound B (3 mg/kg and 10 mg/kg). By day 11, the IMI+Vehicle group exhibited significantly higher scores for the presence of induration on the skin at the area of induction of psoriasis relative to all treatment groups, and this difference continued for the remainder of the study. By day 10 (after two days of treatment), animals in the IMI+Vehicle treatment group exhibited significantly higher scores for peeling on the skin at the area of induction of psoriasis relative to all other treatment groups, and this difference continued for the remainder of the study. Mean scores for the presence of erythema, presence of induration, and presence of peeling are shown in Tables 17C, 17D, and 17E, respectively, and represented in FIGS. 9E, 9F, and 9G, respectively. It was shown that administration of Compounds A and B significantly reduced the presence of erythema on the skin, and induration and peeling of the skin at the area of induction.

With reference to the study on the effects of administration of the three different doses of Compound A and Compound B, significant differences were observed between the mean scores of the presence of peeling on the skin at the area of induction of psoriasis on mice of the treatment groups between Day 8 (corresponding to the first day of the start of treatment) until Day 15 (corresponding to the last of the experiment, 24 hours after the last treatment was administered). For Day 8, before the start of treatment, the mean scores of the severity of psoriasis at the area of induction of psoriasis on the mice of the Vehicle+Vehicle group was significantly lower than the scores of the IMI+Vehicle, and IMI+Compound A, and IMI+Compound B groups. For Day 9, the mean scores of the severity of psoriasis at the area of induction of psoriasis on mice in the Vehicle+Vehicle group was significantly lower than the scores for mice in the IMI+Vehicle, IMI+Compound A, and IMI+Compound B groups, and the mean scores of the IMI+Vehicle group was significantly higher than the scores of the IMI+Compound A groups (all doses), and the two higher doses of the IMI+Compound B groups (3 mg/kg, 10 mg/kg). For Days 10, 11, 12, 13, and 14, the mean scores of the severity of psoriasis of the Vehicle+Vehicle group were significantly lower than the mean scores of the IMI+Vehicle, IMI+Compound A, and IMI+Compound B groups, and the mean scores of the IMI+Vehicle groups were significantly higher than the mean scores for the IMI+Compound A (all doses) and IMI+Compound B (all doses) treatment groups. For Day 15, the mean score of the severity of psoriasis of the Vehicle+Vehicle group was significantly lower than the mean scores for the IMI+Vehicle, IMI+Compound A (1 mg/kg), and IMI+Compound B (all doses) treatment groups, and the mean score of the severity of psoriasis for the IMI+Vehicle group was significantly higher than the mean score for the IMI+Compound A (all doses) and IMI+Compound B (all doses) treatment groups.

TABLE 17C
Mean scores of the presence of erythema on the skin of animals at the area of induction of psoriasis
(mean ± SEM).

| Group | D8 | D9 | D10 | D11 | D12 | D13 | D14 | D15 |
|---|---|---|---|---|---|---|---|---|
| 1 (Vehicle + | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 |
| Vehicle) | | | | | | | | |
| 2 (IMI + | 2.1 ± 0.1 | 3.1 ± 0.2 | 3.0 ± 0.0 | 3.4 ± 0.2 | 3.4 ± 0.2 | 3.4 ± 0.2 | 3.6 ± 0.2 | 3.6 ± 0.2 |
| Vehicle)* | | | | | | | | |
| 3 (IMI + | 2.0 ± 0.0 | 2.0 ± 0.3 | 1.75 ± 0.2 | 1.9 ± 0.1 | 1.5 ± 0.2 | 0.5 ± 0.2 | 0.3 ± 0.2 | 0.0 ± 0.0 |
| Compound A 1 | | (p = 0.024) | (p < 0.001) | (p < 0.001) | (p < 0.001) | (p < 0.001) | (p < 0.001) | (p < 0.001) |
| mg/kg) | | | | | | | | |
| 4 (IMI + | 1.9 ± 0.1 | 1.6 ± 0.2 | 1.4 ± 0.2 | 1.0 ± 0.0 | 0.8 ± 0.2 | 0.3 ± 0.2 | 0.3 ± 0.2 | 0.0 ± 0.0 |
| Compound A 3 | | (p = 0.001) | (p < 0.001) | (p < 0.001) | (p < 0.001) | (p < 0.001) | (p < 0.001) | (p < 0.001) |
| mg/kg) | | | | | | | | |
| 5 (IMI + | 2.3 ± 0.2 | 1.2 ± 0.2 | 1.0 ± 0.2 | 1.3 ± 0.2 | 0.5 ± 0.2 | 0.1 ± 0.1 | 0.0 ± 0.0 | 0.0 ± 0.0 |
| Compound | | (p < 0.001) | (p < 0.001) | (p < 0.001) | (p < 0.001) | (p < 0.001) | (p < 0.001) | (p < 0.001) |
| A 10 mg/kg) | | | | | | | | |
| 6 (IMI + | 2.1 ± 0.1 | 1.8 ± 0.2 | 1.8 ± 0.2 | 2.3 ± 0.2 | 1.8 ± 0.2 | 1.5 ± 0.3 | 1.8 ± 0.3 | 1.5 ± 0.2 |
| Compound B 1 | | (p = 0.002) | (p < 0.001) | (p = 0.001) | (p < 0.001) | (p = 0.001) | (p = 0.001) | (p = 0.001) |
| mg/kg) | | | | | | | | |
| 7 (IMI + | 2.4 ± 0.2 | 1.9 ± 0.3 | 1.6 ± 0.2 | 1.9 ± 0.2 | 1.9 ± 0.1 | 1.5 ± 0.2 | 1.3 ± 0.2 | 0.9 ± 0.2 |
| Compound B 3 | | (p = 0.01) | (p < 0.001) | (p = 0.003) | (p < 0.001) | (p < 0.001) | (p < 0.001) | (p < 0.001) |
| mg/kg) | | | | | | | | |
| 8 (IMI + | 2.1 ± 0.1 | 1.5 ± 0.2 | 1.8 ± 0.2 | 1.5 ± 0.2 | 0.9 ± 0.3 | 0.6 ± 0.3 | 0.1 ± 0.1 | 0.1 ± 0.1 |
| Compound | | (p = 0.001) | (p < 0.001) | (p < 0.001) | (p < 0.001) | (p < 0.001) | (p < 0.001) | (p < 0.001) |
| B 10 mg/kg) | | | | | | | | |
| 9 (IMI + | 2.1 ± 0.1 | 1.5 ± 0.2 | 1.6 ± 0.2 | 1.4 ± 0.2 | 0.75 ± 0.2 | 0.3 ± 0.2 | 0.0 ± 0.0 | 0.1 ± 0.1 |
| Apremilast | | (p = 0.001) | (p < 0.001) | (p < 0.001) | (p < 0.001) | (p < 0.001) | (p < 0.001) | (p < 0.001) |
| 3 mg/kg) | | | | | | | | |
| 10 (IMI + | 2.1 ± 0.1 | 2.1 ± 0.1 | 2.3 ± 0.2 | 1.8 ± 0.4 | 1.8 ± 0.2 | 0.8 ± 0.3 | 0.2 ± 0.2 | 0.0 ± 0.0 |
| Deucravacitinib | | (p = 0.005) | (p = 0.006) | (p = 0.01) | (p < 0.001) | (p = 0.002) | (p = 0.001) | (p = 0.001) |
| 3 mg/kg) | | | | | | | | |
| 11 (IMI + | 2.3 ± 0.2 | 1.8 ± 0.2 | 1.0 ± 0.2 | 0.3 ± 0.2 | 0.0 ± 0.0 | 0.0 ± 0.0 | 0.0 ± 0.0 | 0.0 ± 0.0 |
| Clobetasol | | (p = 0.002) | (p < 0.001) | (p < 0.001) | (p < 0.001) | (p < 0.001) | (p < 0.001) | (p < 0.001) |
| 0.05%) | | | | | | | | |

*p values indicate significance of a mean score relative to the IMI + Vehicle score.

TABLE 17D
Mean scores of the presence of induration on the skin of animals at the area of induction of psoriasis
(mean ± SEM).

| Group | D8 | D9 | D10 | D11 | D12 | D13 | D14 | D15 |
|---|---|---|---|---|---|---|---|---|
| 1 (Vehicle + | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 |
| Vehicle) | | | | | | | | |
| 2 (IMI + | 2.3 ± 0.3 | 2.8 ± 0.2 | 2.8 ± 0.2 | 3.3 ± 0.2 | 3.1 ± 0.2 | 3.1 ± 0.1 | 3.0 ± 0.0 | 3.1 ± 0.1 |
| Vehicle)* | | | | | | | | |
| 3 (IMI + | 2.6 ± 0.2 | 2.4 ± 0.2 | 2.1 ± 0.2 | 2.0 ± 0.3 | 1.6 ± 0.2 | 1.5 ± 0.2 | 0.9 ± 0.2 | 0.5 ± 0.2 |
| Compound A 1 | | | | (p = 0.004) | (p = 0.001) | (p < 0.001) | (p < 0.001) | (p < 0.001) |
| mg/kg) | | | | | | | | |
| 4 (IMI + | 2.5 ± 0.2 | 2.6 ± 0.2 | 2.1 ± 0.1 | 1.4 ± 0.2 | 1.4 ± 0.2 | 0.6 ± 0.2 | 0.3 ± 0.2 | 0.1 ± 0.1 |
| Compound A 3 | | | (p = 0.017) | (p < 0.001) | (p = 0.001) | (p < 0.001) | (p < 0.001) | (p < 0.001) |
| mg/kg) | | | | | | | | |
| 5 (IMI + | 2.6 ± 0.2 | 2.0 ± 0.3 | 1.8 ± 0.2 | 1.6 ± 0.2 | 1.1 ± 0.2 | 0.8 ± 0.2 | 0.4 ± 0.2 | 0.1 ± 0.1 |
| Compound A 10 | | (p = 0.043) | (p = 0.003) | (p < 0.001) | (p < 0.001) | (p < 0.001) | (p < 0.001) | (p < 0.001) |
| mg/kg) | | | | | | | | |
| 6 (IMI + | 2.5 ± 0.2 | 2.5 ± 0.2 | 2.3 ± 0.2 | 2.3 ± 0.3 | 2.0 ± 0.0 | 2.0 ± 0.0 | 2.0 ± 0.0 | 1.9 ± 0.1 |
| Compound B 1 | | | | (p = 0.009) | (p = 0.001) | (p < 0.001) | (p < 0.001) | (p < 0.001) |
| mg/kg) | | | | | | | | |
| 7 (IMI + | 2.6 ± 0.2 | 2.0 ± 0.0 | 2.1 ± 0.1 | 1.7 ± 0.2 | 1.9 ± 0.1 | 1.9 ± 0.1 | 1.9 ± 0.1 | 1.4 ± 0.2 |
| Compound B 3 | | (p = 0.003) | (p = 0.017) | (p < 0.001) | (p = 0.001) | (p < 0.001) | (p < 0.001) | (p < 0.001) |
| mg/kg) | | | | | | | | |
| 8 (IMI + | 2.8 ± 0.2 | 2.0 ± 0.2 | 2.1 ± 0.2 | 1.5 ± 0.2 | 1.1 ± 0.1 | 1.0 ± 0.0 | 0.6 ± 0.2 | 0.4 ± 0.2 |
| Compound B 10 | | (p = 0.016) | | (p < 0.001) | (p < 0.001) | (p < 0.001) | (p < 0.001) | (p < 0.001) |
| mg/kg) | | | | | | | | |
| 9 (IMI + | 3.0 ± 0.0 | 2.3 ± 0.2 | 2.0 ± 0.2 | 1.8 ± 0.2 | 0.9 ± 0.2 | 0.4 ± 0.2 | 0.4 ± 0.2 | 0.1 ± 0.1 |
| Apremilast 3 | | | (p = 0.016) | (p < 0.001) | (p < 0.001) | (p < 0.001) | (p < 0.001) | (p < 0.001) |
| mg/kg) | | | | | | | | |
| 10 (IMI + | 2.8 ± 0.3 | 2.4 ± 0.2 | 2.0 ± 0.2 | 1.5 ± 0.2 | 0.5 ± 0.2 | 0.0 ± 0.0 | 0.0 ± 0.0 | 0.0 ± 0.0 |
| Deucravacitinib | | | (p = 0.024) | (p = 0.001) | (p = 0.002) | (p < 0.001) | (p < 0.001) | (p < 0.001) |
| 3 mg/kg) | | | | | | | | |
| 11 (IMI + | 2.8 ± 0.2 | 1.3 ± 0.2 | 1.0 ± 0.0 | 0.0 ± 0.0 | 0.0 ± 0.0 | 0.0 ± 0.0 | 0.0 ± 0.0 | 0.0 ± 0.0 |
| Clobetasol | | (p = 0.001) | (p < 0.001) | (p < 0.001) | (p < 0.001) | (p < 0.001) | (p < 0.001) | (p < 0.001) |
| 0.05%) | | | | | | | | |

*p values indicate significance of a mean score relative to the IMI + Vehicle score.

TABLE 17E
Mean scores of the presence of peeling on the skin of animals at the area of induction of psoriasis
(mean ± SEM).

| Group | D8 | D9 | D10 | D11 | D12 | D13 | D14 | D15 |
|---|---|---|---|---|---|---|---|---|
| 1 (Vehicle + | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 |
| Vehicle) | | | | | | | | |
| 2 (IMI + Vehicle)* | 2.5 ± 0.2 | 2.6 ± 0.2 | 2.9 ± 0.2 | 3.0 ± 0.0 | 2.9 ± 0.1 | 3.0 ± 0.2 | 3.0 ± 0.0 | 3.3 ± 0.2 |
| 3 (IMI + Compound | 2.6 ± 0.2 | 2.6 ± 0.2 | 2.1 ± 0.1 | 1.9 ± 0.2 | 1.3 ± 0.2 | 1.1 ± 0.1 | 1.0 ± 0.0 | 0.7 ± 0.2 |
| A 1 mg/kg) | | | (p = 0.017) | (p = 0.001) | (p < 0.001) | (p < 0.001) | (p < 0.001) | (p < 0.001) |
| 4 (IMI + Compound | 2.3 ± 0.2 | 2.5 ± 0.2 | 2.0 ± 0.2 | 1.6 ± 0.2 | 1.4 ± 0.2 | 0.9 ± 0.1 | 0.3 ± 0.2 | 0.3 ± 0.2 |
| A 3 mg/kg) | | | (p = 0.015) | (p < 0.001) | (p < 0.001) | (p < 0.001) | (p < 0.001) | (p < 0.001) |
| 5 (IMI + Compound | 2.3 ± 0.3 | 2.0 ± 0.3 | 1.5 ± 0.2 | 1.5 ± 0.2 | 1.3 ± 0.2 | 0.6 ± 0.2 | 0.3 ± 0.2 | 0.3 ± 0.2 |
| A 10 mg/kg) | | | (p = 0.002) | (p < 0.001) | (p < 0.001) | (p < 0.001) | (p < 0.001) | (p < 0.001) |
| 6 (IMI + Compound | 2.1 ± 0.1 | 2.5 ± 0.3 | 2.0 ± 0.2 | 2.0 ± 0.1 | 1.9 ± 0.1 | 2.0 ± 0.3 | 1.9 ± 0.2 | 1.8 ± 0.3 |
| B 1 mg/kg) | | | (p = 0.015) | (p = 0.001) | (p < 0.001) | (p = 0.014) | (p < 0.001) | (p = 0.002) |
| 7 (IMI + Compound | 2.6 ± 0.3 | 2.1 ± 0.0 | 2.0 ± 0.2 | 1.9 ± 0.1 | 1.6 ± 0.2 | 1.6 ± 0.2 | 1.5 ± 0.1 | 1.3 ± 0.3 |
| B 3 mg/kg) | | | (p = 0.015) | (p < 0.001) | (p = 0.001) | (p = 0.001) | (p < 0.001) | (p < 0.001) |
| 8 (IMI + Compound | 2.4 ± 0.3 | 2.1 ± 0.2 | 2.0 ± 0.3 | 1.9 ± 0.1 | 1.0 ± 0.2 | 0.4 ± 0.2 | 0.5 ± 0.2 | 0.4 ± 0.2 |
| B 10 mg/kg) | | | (p = 0.037) | (p < 0.001) | (p = 0.001) | (p < 0.001) | (p < 0.001) | (p < 0.001) |
| 9 (IMI + Apremilast | 2.5 ± 0.2 | 2.1 ± 0.2 | 1.6 ± 0.3 | 1.4 ± 0.2 | 1.1 ± 0.1 | 0.3 ± 0.2 | 0.4 ± 0.2 | 0.1 ± 0.1 |
| 3 mg/kg) | | | (p = 0.008) | (p < 0.001) | (p < 0.001) | (p < 0.001) | (p < 0.001) | (p < 0.001) |
| 10 (IMI + | 2.5 ± 0.2 | 2.1 ± 0.1 | 1.9 ± 0.1 | 2.0 ± 0.3 | 1.0 ± 0.0 | 0.8 ± 0.3 | 0.8 ± 0.2 | 0.3 ± 0.2 |
| Deucravacitinib 3 | | | (p = 0.006) | (p = 0.003) | (p < 0.001) | (p = 0.002) | (p < 0.001) | (p = 0.001) |
| mg/kg) | | | | | | | | |
| 11 (IMI + | 2.4 ± 0.2 | 1.8 ± 0.3 | 0.9 ± 0.1 | 0.4 ± 0.2 | 0.8 ± 0.2 | 0.4 ± 0.2 | 0.1 ± 0.1 | 0.1 ± 0.1 |
| Clobetasol 0.05%) | | (p = 0.024) | (p < 0.001) | (p < 0.001) | (p < 0.001) | (p < 0.001) | (p < 0.001) | (p < 0.001) |

*p values indicate significance of a mean score relative to the IMI + Vehicle score.

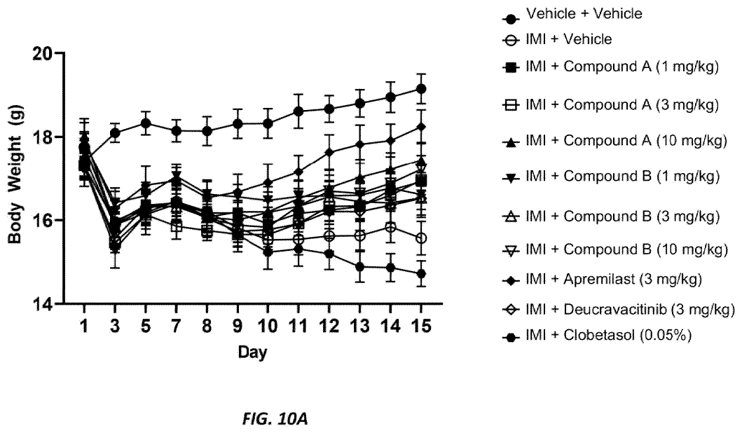

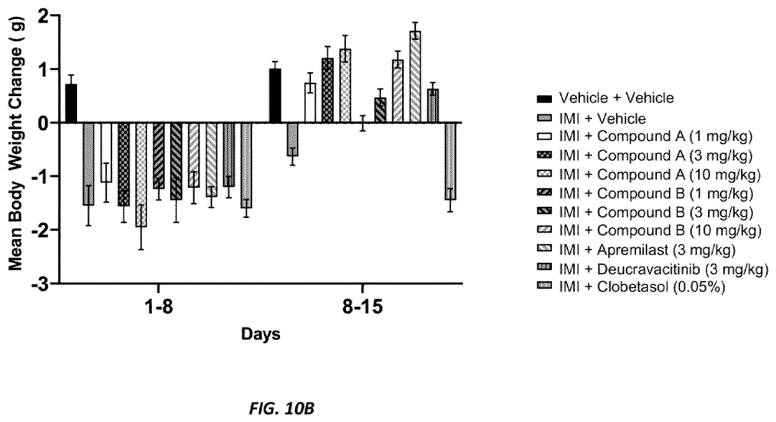

FIGS. 10A-10B and Table 17F depict body weight changes in animals over the course of the study (FIG. 10A), and mean body weight changes over the first 8 days and the last 7 days of the study (FIG. 10B). All animals where psoriasis was induced by administration of imiquimod lost weight over the first three days of the study. Animals receiving Compound A exhibited a modest gain in body weight over the remaining days of the study, with ending body weights being just below starting weights. Animals receiving the lower two doses of Compound B exhibited slightly lower body weight gains following the initial weight loss period, and the 10 mg/kg dose of Compound 1 proved most effective at limiting weight loss. Specifically, the mean weight gain for animals in the IMI+Vehicle group was significantly lower than animals receiving Compound A (all doses) and Compound B (3 mg/kg and 10 mg/kg doses). It is noted that all three tested doses of Compound A were more effective at limiting weight loss than both deucravacitinib and clobetasol. Similarly, animals receiving Compound A (all doses) or Compound B3 (10 mg/kg) exhibited significantly higher mean body weights compared to animals in the IMI+Vehicle treatment group. While all animals where psoriasis was induced lost weight over the first 8 days, all animals treated with Compound A exhibited weight gains over the last 7 days (FIG. 10B). This is also true for the 3 mg/kg dose and 10 mg/kg dose of Compound B.

TABLE 17F
Body Weight in IMI-Psoriasis Model.

| | Animal | | | | | | | | | | | | | MBWC | MBWC |
|---|---|---|---|---|---|---|---|---|---|---|---|---|---|---|---|
| Group | Number# | D1 | D3 | D5 | D7 | D8 | D9 | D10 | D11 | D12 | D13 | D14 | D15 | D1-D8 | D8-D15 |
| 1 (Vehicle + Vehicle) | 1 | 17.79 | 17.82 | 18.07 | 17.80 | 17.97 | 17.78 | 18.35 | 18.39 | 18.48 | 18.95 | 19.06 | 19.13 | 0.18 | 1.16 |
| | 2 | 17.85 | 17.51 | 18.45 | 18.23 | 18.04 | 17.87 | 18.36 | 18.31 | 18.55 | 19.05 | 19.16 | 19.17 | 0.19 | 1.13 |
| | 3 | 17.53 | 18.09 | 18.55 | 18.37 | 17.73 | 18.20 | 18.64 | 17.34 | 18.72 | 18.75 | 18.93 | 18.94 | 0.20 | 1.21 |
| | 4 | 15.32 | 17.48 | 17.31 | 17.29 | 16.63 | 17.43 | 17.31 | 18.77 | 17.61 | 17.92 | 17.83 | 18.09 | 1.31 | 1.46 |
| | 5 | 16.82 | 18.17 | 17.73 | 17.38 | 17.71 | 17.96 | 17.63 | 17.98 | 18.26 | 17.85 | 17.79 | 18.16 | 0.89 | 0.45 |
| | 6 | 16.95 | 17.85 | 17.83 | 17.83 | 18.13 | 17.69 | 17.40 | 17.85 | 18.31 | 18.28 | 18.48 | 18.89 | 1.18 | 0.76 |
| | 7 | 19.14 | 19.47 | 19.84 | 19.58 | 19.98 | 20.37 | 20.44 | 21.09 | 20.73 | 20.78 | 21.04 | 21.30 | 0.84 | 1.32 |
| | 8 | 17.93 | 18.37 | 18.84 | 18.68 | 18.93 | 19.21 | 18.44 | 19.16 | 18.69 | 18.83 | 19.30 | 19.51 | 1.00 | 0.58 |
| 2 (IMI + Vehicle) | 1 | 17.20 | 15.21 | 15.54 | 14.81 | 15.61 | 15.57 | 15.24 | 15.05 | 15.03 | 15.18 | 15.23 | 14.98 | −1.59 | −0.63 |
| | 2 | 18.41 | 17.15 | 17.63 | 17.55 | 17.46 | 16.87 | 16.55 | 16.40 | 16.16 | 16.41 | 16.52 | 16.02 | −0.95 | −1.44 |
| | 3 | 17.58 | 15.43 | 15.54 | 15.12 | 14.65 | 14.18 | 14.23 | 14.00 | 14.55 | 14.33 | 14.12 | 13.92 | −2.93 | −0.73 |
| | 4 | 18.07 | 15.73 | 16.16 | 16.73 | 16.20 | 15.84 | 15.53 | 15.33 | 14.96 | 15.27 | 15.55 | 15.26 | −1.87 | −0.94 |
| | 5 | 16.29 | 14.54 | 14.70 | 15.37 | 15.06 | 15.08 | 14.45 | 14.48 | 14.54 | 14.62 | 14.89 | 14.71 | −1.23 | −0.35 |
| | 6 | 18.90 | 16.33 | 16.67 | 16.49 | 15.82 | 15.59 | 15.21 | 15.54 | 15.71 | 15.89 | 16.54 | 15.91 | −3.08 | 0.09 |
| | 7 | 16.90 | 16.60 | 16.76 | 17.21 | 16.83 | 16.31 | 16.40 | 16.41 | 16.57 | 15.99 | 16.53 | 16.04 | −0.07 | −0.79 |
| | 8 | 18.68 | 16.20 | 17.15 | 18.24 | 17.99 | 17.04 | 16.58 | 17.10 | 17.42 | 17.30 | 17.30 | 17.70 | −0.69 | −0.29 |
| 3 (IMI + Compound A 1 | 1 | 16.93 | 15.40 | 15.20 | 15.18 | 14.60 | 14.63 | 14.70 | 14.84 | 14.87 | 15.05 | 15.56 | 15.74 | −2.33 | 1.14 |
| mg/kg) | 2 | 17.80 | 16.02 | 16.76 | 17.36 | 16.99 | 17.16 | 16.93 | 17.22 | 16.99 | 17.40 | 17.50 | 17.76 | −0.81 | 0.77 |
| | 3 | 16.08 | 15.09 | 15.65 | 15.85 | 15.82 | 15.82 | 15.61 | 15.81 | 15.46 | 16.20 | 16.49 | 16.69 | −0.26 | 0.87 |
| | 4 | 16.97 | 15.92 | 16.45 | 16.10 | 16.44 | 16.15 | 15.89 | 15.74 | 15.54 | 15.94 | 16.50 | 17.05 | −0.53 | 0.61 |
| | 5 | 17.74 | 16.29 | 16.71 | 16.71 | 16.60 | 16.59 | 16.21 | 16.25 | 16.99 | 16.75 | 17.20 | 17.07 | −1.14 | 0.47 |
| | 6 | 17.12 | 16.59 | 17.23 | 17.27 | 17.49 | 17.04 | 16.73 | 16.90 | 17.30 | 17.05 | 17.24 | 17.34 | 0.37 | −0.15 |
| | 7 | 18.36 | 16.98 | 17.40 | 17.41 | 16.66 | 16.41 | 16.12 | 16.89 | 17.06 | 16.69 | 16.86 | 17.23 | −1.70 | 0.57 |
| | 8 | 17.34 | 15.19 | 15.42 | 15.38 | 14.75 | 15.55 | 15.11 | 15.58 | 15.66 | 15.46 | 16.35 | 16.41 | −2.59 | 1.66 |
| 4 (IMI + Compound A 3 | 1 | 17.31 | 15.94 | 16.84 | 16.64 | 16.59 | 16.53 | 16.64 | 17.05 | 17.56 | 17.68 | 17.80 | 18.44 | −0.72 | 1.85 |
| mg/kg) | 2 | 16.98 | 14.69 | 15.15 | 14.71 | 14.84 | 14.49 | 14.53 | 14.95 | 15.38 | 16.03 | 16.01 | 16.47 | −2.14 | 1.63 |
| | 3 | 19.05 | 16.63 | 16.74 | 16.54 | 16.76 | 16.98 | 17.40 | 17.25 | 17.88 | 18.03 | 17.97 | 18.44 | −2.29 | 1.68 |
| | 4 | 16.34 | 15.29 | 15.65 | 14.84 | 15.51 | 15.40 | 15.71 | 15.60 | 15.18 | 15.39 | 15.32 | 15.82 | −0.83 | 0.31 |
| | 5 | 18.16 | 15.93 | 17.11 | 17.16 | 15.95 | 16.26 | 16.02 | 16.07 | 16.82 | 16.24 | 16.91 | 16.95 | −2.21 | 1.00 |
| | 6 | 17.71 | 15.05 | 16.06 | 15.73 | 15.14 | 15.22 | 15.31 | 15.67 | 16.07 | 16.09 | 16.84 | 16.91 | −2.57 | 1.77 |
| | 7 | 15.90 | 14.65 | 16.09 | 15.75 | 15.51 | 15.49 | 14.80 | 15.43 | 15.80 | 15.63 | 15.90 | 16.13 | −0.39 | 0.62 |
| | 8 | 17.04 | 15.53 | 15.35 | 15.45 | 15.67 | 14.89 | 14.76 | 15.33 | 15.88 | 15.61 | 15.97 | 16.47 | −1.37 | 0.80 |
| 5 (IMI + Compound A | 1 | 17.49 | 15.01 | 15.12 | 16.33 | 16.07 | 15.69 | 15.53 | 15.74 | 15.83 | 17.35 | 16.58 | 16.60 | −1.42 | 0.53 |
| 10 mg/kg) | 2 | 17.30 | 14.91 | 14.82 | 15.77 | 15.40 | 15.96 | 15.84 | 15.58 | 15.88 | 16.03 | 16.42 | 16.43 | −1.90 | 1.03 |
| | 3 | 19.10 | 17.43 | 17.32 | 17.25 | 17.12 | 16.63 | 16.68 | 16.84 | 17.03 | 17.55 | 17.59 | 18.35 | −1.98 | 1.23 |
| | 4 | 19.20 | 18.70 | 19.02 | 18.62 | 18.84 | 19.27 | 18.91 | 19.15 | 19.25 | 19.51 | 19.36 | 19.39 | −0.36 | 0.55 |
| | 5 | 19.68 | 15.42 | 15.82 | 16.18 | 15.35 | 15.91 | 16.27 | 16.81 | 17.13 | 16.98 | 17.68 | 17.81 | −4.33 | 2.46 |
| | 6 | 17.68 | 17.04 | 16.99 | 17.36 | 16.21 | 16.41 | 16.73 | 17.05 | 17.19 | 17.10 | 17.84 | 18.26 | −1.47 | 2.05 |
| | 7 | 16.28 | 13.99 | 15.18 | 15.18 | 14.88 | 14.96 | 15.10 | 15.58 | 16.27 | 16.02 | 16.59 | 16.77 | −1.40 | 1.89 |
| | 8 | 17.45 | 14.57 | 15.12 | 14.93 | 14.68 | 14.71 | 14.49 | 15.32 | 15.58 | 15.65 | 15.62 | 15.96 | −2.77 | 1.28 |
| 6 (IMI + Compound B 1 | 1 | 17.85 | 16.05 | 15.71 | 16.02 | 15.83 | 15.70 | 15.94 | 16.10 | 16.03 | 16.53 | 16.38 | 16.34 | −2.02 | 0.51 |
| mg/kg) | 2 | 16.71 | 15.75 | 15.27 | 15.76 | 15.34 | 15.29 | 15.03 | 14.61 | 14.93 | 14.84 | 14.75 | 14.67 | −1.37 | −0.67 |
| | 3 | 18.14 | 16.34 | 17.54 | 17.96 | 17.54 | 17.68 | 17.60 | 17.64 | 17.80 | 18.05 | 18.20 | 18.01 | −0.60 | 0.47 |
| | 4 | 17.67 | 16.73 | 16.77 | 17.02 | 16.58 | 16.64 | 16.38 | 16.08 | 16.29 | 16.45 | 16.97 | 16.62 | −1.09 | 0.04 |
| | 5 | 18.64 | 17.52 | 17.97 | 17.69 | 17.61 | 17.26 | 17.13 | 17.41 | 17.39 | 17.04 | 17.80 | 17.29 | −1.03 | −0.32 |
| | 6 | 18.37 | 16.25 | 16.83 | 17.48 | 16.23 | 15.94 | 16.08 | 16.29 | 16.49 | 16.34 | 16.16 | 16.44 | −2.14 | 0.21 |
| | 7 | 17.36 | 15.31 | 15.98 | 16.75 | 16.35 | 16.69 | 15.92 | 16.64 | 16.17 | 16.45 | 16.37 | 16.12 | −1.01 | −0.23 |
| | 8 | 18.14 | 17.36 | 16.57 | 17.68 | 17.45 | 17.21 | 17.66 | 17.72 | 17.53 | 17.04 | 17.46 | 17.36 | −0.69 | −0.09 |
| 7 (IMI + Compound B 3 | 1 | 16.04 | 13.11 | 14.01 | 14.25 | 13.57 | 13.93 | 14.23 | 13.97 | 14.44 | 13.94 | 13.70 | 13.78 | −2.47 | 0.21 |
| mg/kg) | 2 | 18.93 | 16.65 | 16.29 | 16.26 | 15.67 | 15.56 | 15.76 | 16.26 | 16.57 | 16.60 | 16.12 | 16.88 | −3.26 | 1.21 |
| | 3 | 17.71 | 15.84 | 15.26 | 15.31 | 15.44 | 15.22 | 15.76 | 15.22 | 15.46 | 15.54 | 15.70 | 15.93 | −2.27 | 0.49 |
| | 4 | 17.81 | 16.79 | 17.65 | 17.34 | 17.59 | 17.78 | 17.87 | 17.70 | 17.60 | 17.74 | 17.96 | 18.05 | −0.22 | 0.46 |
| | 5 | 16.63 | 15.99 | 16.66 | 16.40 | 16.51 | 16.23 | 16.28 | 16.63 | 17.05 | 16.71 | 17.04 | 17.29 | −0.12 | 0.78 |
| | 6 | 17.34 | 16.64 | 17.47 | 17.79 | 17.07 | 17.14 | 17.32 | 16.90 | 16.97 | 17.05 | 17.47 | 17.08 | −0.27 | 0.01 |
| | 7 | 17.52 | 14.93 | 16.35 | 16.44 | 16.13 | 15.89 | 15.99 | 16.81 | 16.65 | 16.35 | 16.08 | 15.94 | −1.39 | −0.19 |
| | 8 | 18.08 | 16.90 | 16.80 | 16.80 | 16.51 | 16.28 | 16.22 | 17.13 | 17.68 | 17.58 | 17.19 | 17.29 | −1.57 | 0.78 |
| 8 (IMI + Compound B | 1 | 18.66 | 16.89 | 16.85 | 17.09 | 16.51 | 16.61 | 16.48 | 16.44 | 16.63 | 17.12 | 17.36 | 18.01 | −2.15 | 1.50 |
| 10 mg/kg) | 2 | 16.97 | 15.12 | 16.05 | 15.74 | 15.56 | 15.20 | 15.64 | 15.87 | 16.40 | 16.52 | 16.91 | 16.93 | −1.41 | 1.37 |
| | 3 | 17.99 | 16.64 | 17.14 | 18.31 | 18.14 | 17.85 | 18.33 | 17.94 | 18.54 | 18.44 | 18.32 | 18.51 | 0.15 | 0.37 |
| | 4 | 16.59 | 15.35 | 16.55 | 16.60 | 16.14 | 15.85 | 16.10 | 16.30 | 16.96 | 16.93 | 17.28 | 17.31 | −0.45 | 1.17 |
| | 5 | 14.70 | 12.66 | 13.95 | 14.04 | 14.02 | 13.92 | 13.59 | 14.37 | 14.74 | 14.68 | 15.12 | 15.47 | −0.68 | 1.45 |
| | 6 | 18.45 | 15.12 | 16.19 | 16.64 | 16.16 | 15.86 | 15.70 | 16.54 | 16.69 | 16.17 | 16.21 | 16.86 | −2.29 | 0.70 |
| | 7 | 16.88 | 15.18 | 15.85 | 16.00 | 15.50 | 15.31 | 15.00 | 16.26 | 16.23 | 16.27 | 16.48 | 16.66 | −1.38 | 1.16 |
| | 8 | 17.79 | 15.47 | 16.46 | 16.64 | 16.26 | 16.41 | 15.80 | 16.95 | 17.34 | 16.95 | 17.35 | 17.96 | −1.53 | 1.70 |
| 9 (IMI + Apremilast 3 | 1 | 18.04 | 15.91 | 16.70 | 16.55 | 16.41 | 16.75 | 17.13 | 16.87 | 17.32 | 17.57 | 17.89 | 18.27 | −1.63 | 1.86 |
| mg/kg) | 2 | 18.00 | 16.38 | 17.24 | 16.74 | 16.59 | 17.41 | 17.32 | 17.60 | 18.46 | 18.62 | 18.64 | 18.85 | −1.41 | 2.26 |
| | 3 | 18.05 | 17.39 | 17.88 | 17.25 | 17.27 | 17.44 | 17.55 | 17.50 | 17.58 | 18.39 | 18.56 | 18.94 | −0.78 | 1.67 |
| | 4 | 18.89 | 17.17 | 17.91 | 17.69 | 17.43 | 17.12 | 17.39 | 17.32 | 17.84 | 18.19 | 18.40 | 18.85 | −1.46 | 1.42 |
| | 5 | 15.16 | 13.24 | 13.95 | 15.07 | 13.81 | 13.85 | 13.82 | 14.44 | 14.88 | 14.70 | 15.26 | 15.48 | −1.35 | 1.67 |
| | 6 | 17.91 | 16.46 | 16.38 | 16.92 | 17.05 | 17.04 | 17.24 | 17.59 | 18.39 | 18.17 | 17.85 | 18.37 | −0.86 | 1.32 |
| | 7 | 18.62 | 17.63 | 17.65 | 17.97 | 17.50 | 17.41 | 17.62 | 17.99 | 18.34 | 18.61 | 18.49 | 18.62 | −1.12 | 1.12 |
| | 8 | 18.72 | 16.03 | 16.94 | 17.32 | 16.19 | 16.34 | 17.09 | 17.89 | 18.22 | 18.32 | 18.20 | 18.57 | −2.53 | 2.38 |
| 10 (IMI + | 1 | 18.26 | 16.20 | 18.16 | 17.33 | 17.09 | 16.60 | 16.71 | 17.38 | 17.70 | 17.75 | 18.00 | 18.03 | −1.17 | 0.94 |
| Deucravacitinib 3 | 2 | 17.37 | 16.31 | 16.94 | 17.08 | 17.16 | 16.54 | 16.43 | — | — | — | — | — | −0.21 | — |
| mg/kg) | 3 | 16.82 | 14.76 | 15.74 | 15.79 | 15.63 | 15.63 | 16.08 | 15.87 | 15.90 | 16.24 | 16.31 | 16.39 | −1.19 | 0.76 |
| | 4 | 17.07 | 15.24 | 15.23 | 15.48 | 15.50 | 15.57 | 15.84 | 15.51 | 15.59 | 15.77 | 15.59 | 16.00 | −1.57 | 0.50 |
| | 5 | 16.32 | 15.52 | 15.99 | 16.18 | 15.52 | 16.01 | 14.94 | 15.55 | 15.82 | 15.95 | 16.06 | 16.29 | −0.80 | 0.77 |
| | 6 | 18.04 | 15.99 | 17.09 | 17.55 | 17.05 | 16.79 | 16.16 | 16.43 | 17.15 | 16.97 | 17.07 | 17.17 | −0.99 | 0.12 |
| | 7 | 16.47 | 14.97 | 14.94 | 15.22 | 14.52 | 14.75 | 14.20 | 14.72 | 15.06 | 14.52 | 15.08 | 15.22 | −1.95 | 0.70 |
| | 8 | 17.58 | 15.55 | 16.50 | 16.65 | 15.81 | 13.93 | — | — | — | — | — | — | −1.77 | — |
| 11 (IMI + Clobetasol | 1 | 17.07 | 14.93 | 16.15 | 15.51 | 15.82 | 15.95 | 15.11 | 15.27 | 14.64 | 14.45 | 14.55 | 14.30 | −1.25 | −1.52 |
| 0.05%) | 2 | 17.52 | 16.02 | 15.56 | 15.49 | 15.39 | 14.96 | 14.48 | 14.85 | 14.74 | 14.82 | 14.89 | 14.75 | −2.13 | −0.64 |
| | 3 | 19.31 | 17.76 | 18.21 | 18.30 | 18.05 | 17.53 | 17.43 | 17.41 | 17.02 | 16.74 | 16.65 | 16.22 | −1.26 | −1.83 |
| | 4 | 18.57 | 17.57 | 17.74 | 17.72 | 17.68 | 16.98 | 16.38 | 16.38 | 15.95 | 15.71 | 15.49 | 15.41 | −0.89 | −2.27 |
| | 5 | 18.72 | 17.77 | 16.72 | 17.41 | 16.64 | 15.65 | 15.37 | 15.58 | 15.45 | 15.24 | 15.08 | 14.99 | −2.08 | −1.65 |
| | 6 | 16.56 | 14.09 | 15.29 | 15.14 | 14.96 | 14.35 | 14.04 | 13.84 | 13.69 | 13.39 | 13.55 | 13.32 | −1.60 | −1.64 |
| | 7 | 17.64 | 14.81 | 15.64 | 16.24 | 16.14 | 14.99 | 14.71 | 14.92 | 15.73 | 14.65 | 14.66 | 14.54 | −1.50 | −1.60 |
| | 8 | 16.77 | 14.86 | 15.11 | 15.49 | 14.67 | 14.71 | 14.32 | 14.21 | 14.31 | 14.05 | 14.04 | 14.22 | −2.10 | −0.45 |

#Each animal actually had a different number from 1 to 88, and the animal numbers are shown for simplicity as 1-8 for each group.

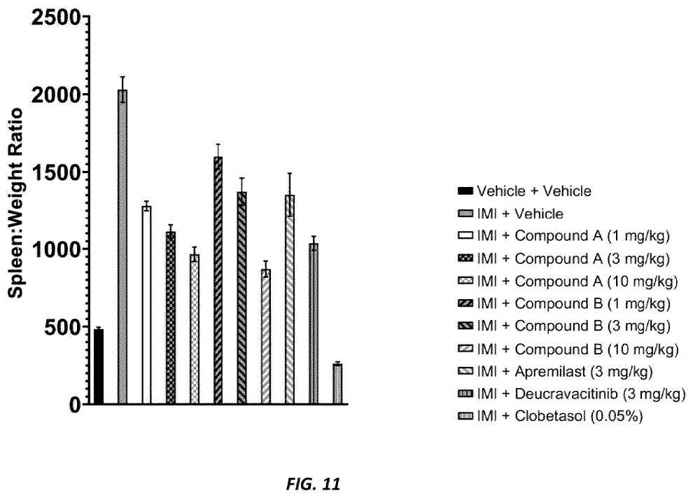

Compound A was also effective at lowering the spleen weight ratio relative to the vehicle treated animal (FIG. 11, Table 17G). There was a dose-dependent decrease in the spleen weight ratio for animals treated both with Compound A and Compound B. All doses of Compound A and the two higher doses of Compound B (3 mg/kg and 10 mg/kg) were able to reduce the spleen weight ratio to a level similar to that of apremilast and deucravacitinib. Notably, the mean spleen weight ratio for animals in the IMI+Vehicle group was significantly higher than the mean spleen weight ratio for animals receiving Compound A (all doses) or Compound B (all doses).

No abnormal behavior of mice was observed during the experiment in the treatment groups except a slight hypoactivity consecutive to each dermal application of Aldara® cream. This was consistent with the behaviour of mice observed in previous experiments on this model of psoriasis.

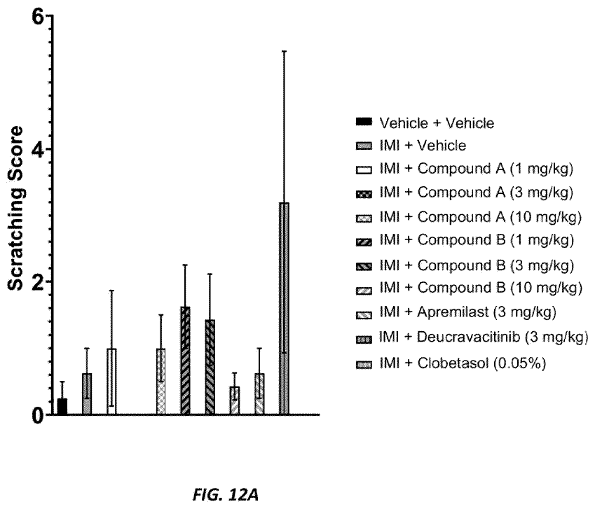

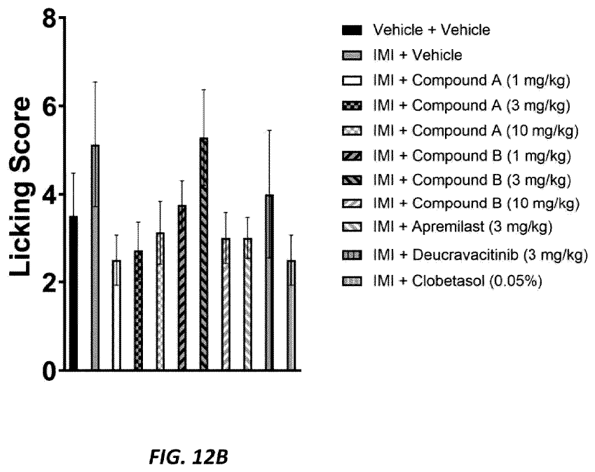

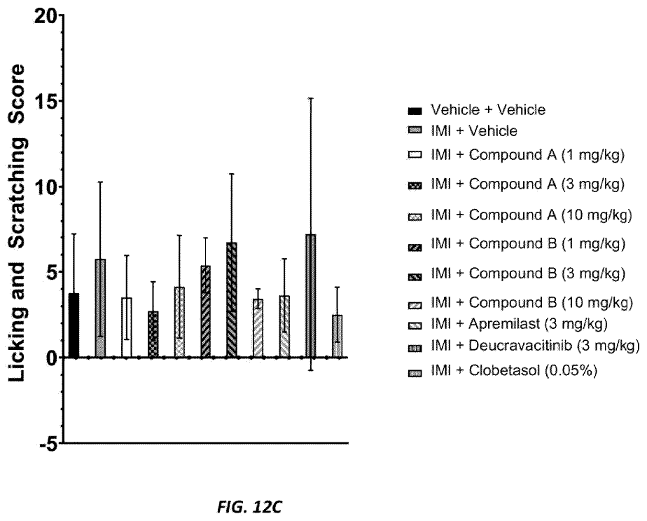

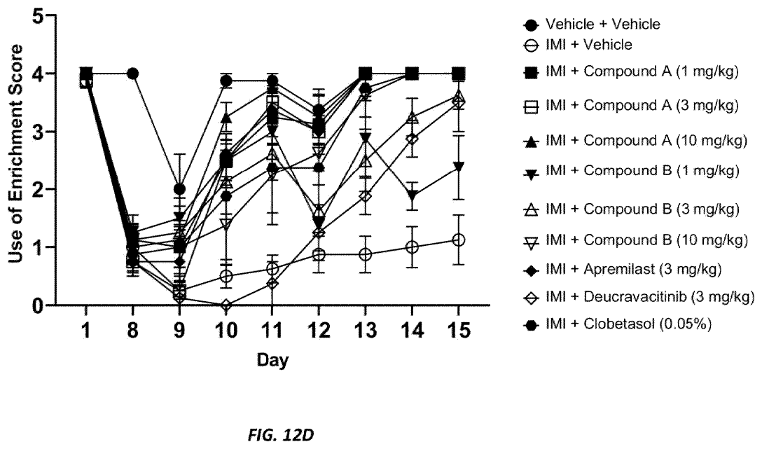

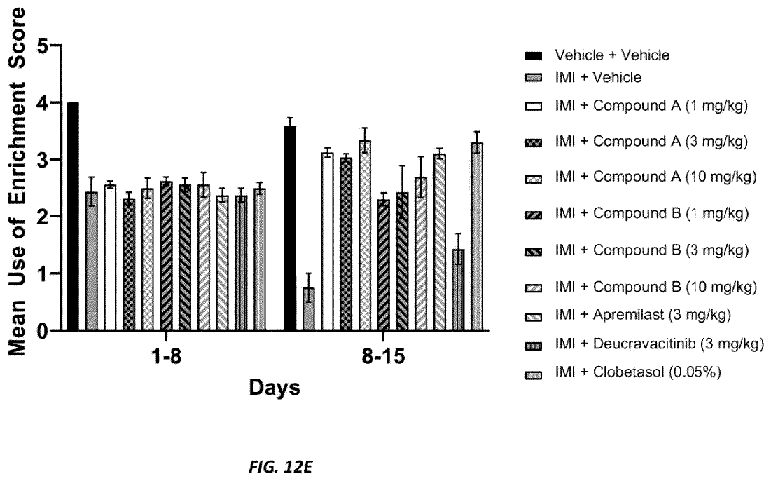

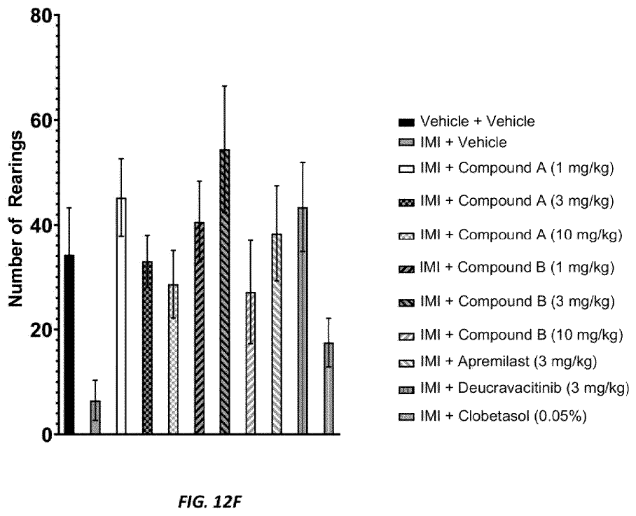

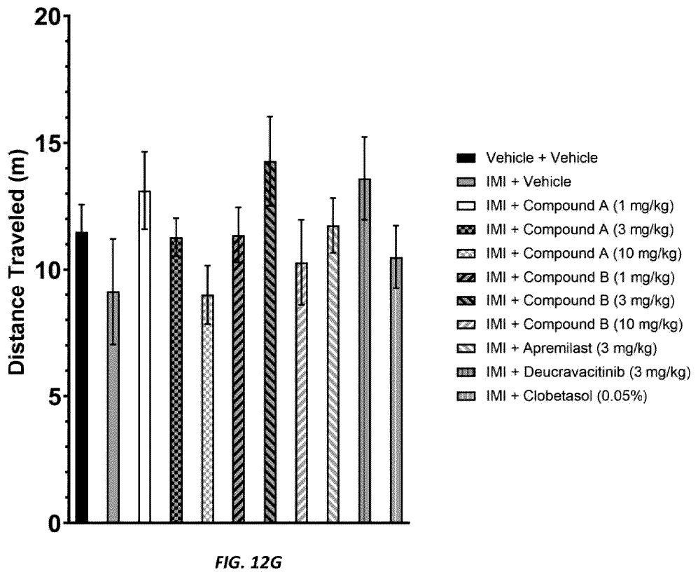

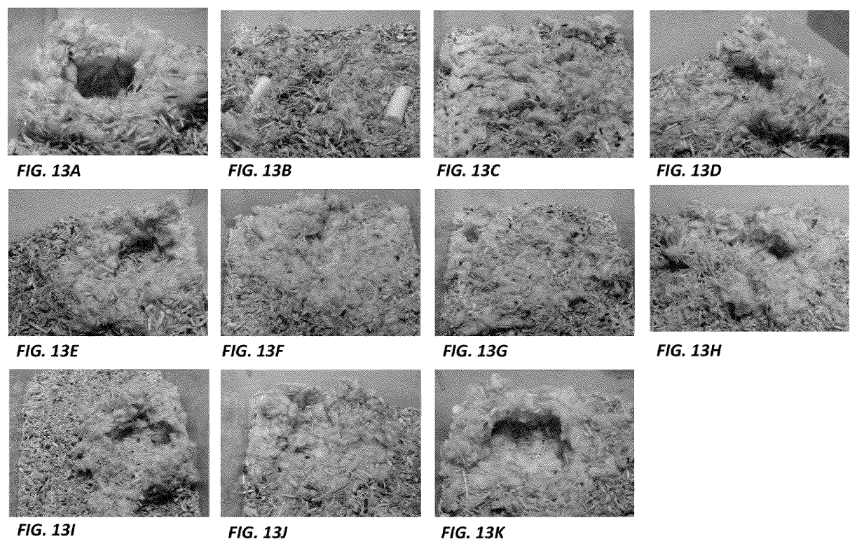

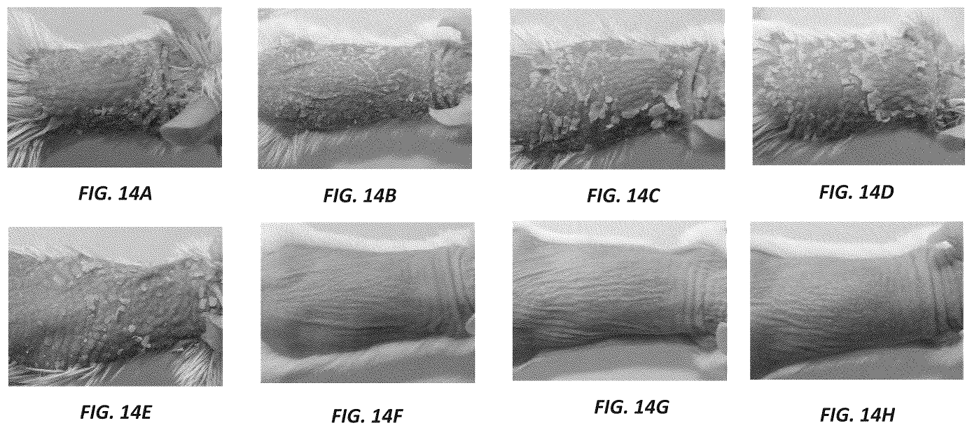

Compound A also proved effective at restoring behavior similar to that observed when psoriasis is not induced (Tables 17H and 17I). FIGS. 12A-12C show scratching and licking scores for animals (Table 17I), and FIGS. 12D-12E show use of enrichment scoring in animals (Table 17H). Although some variability was observed between individual animals with reference to scratching and licking behavior Compound A was effective in reducing licking scoring, as well as improving use of enrichment scores in animals, relative to vehicle-treated animals where psoriasis was induced. No significant difference, however, was observed between the mean numbers of licking and scratching by mice of the treatment groups at the end of the experimentation on Di5 Specifically, regarding enrichment use, the mean use of enrichment score for animals in the IMI+Vehicle group was significantly lower than use of enrichment score of animals receiving either Compound A (all doses) or Compound B (3 mg/kg and 10 mg/kg). Treatment with Compound A further restored rearing behavior (FIG. 12F) and movement (FIG. 12G) in animals relative to vehicle-treated animals where psoriasis was induced (Table 17I). A significant difference was observed between the mean numbers of rearings performed by mice of the treatment groups at the end of the experimentation on D15. Specifically, the mean number of rearings for animals in the IMI+Vehicle group was significantly lower than mean number of rearings for animals receiving either Compound A (all doses) or Compound B (1 mg/kg and 3 mg/kg). FIGS. 13A-13K provide representative images of enriched nesting use in animals treated with Compound A. In vehicle-treated animals, where psoriasis was induced virtually no nesting was used by the animals (FIG. 13B) relative to animals where psoriasis was not induced (FIG. 13A) and nesting use is clearly observed. In contrast, to vehicle treated animals where psoriasis was induced the three doses of Compound A (1 mg/kg, 3 mg/kg, and 10 mg/kg) restored the use of enrichment by animals (FIG. 13C-13E, respectively). Compound B, at the three doses tested (1 mg/kg, 3 mg/kg, and 10 mg/kg), similarly restored use of nesting (FIG. 13F-13H, respectively). Use of nesting by animals treated with apremilast (FIG. 13I), and clobetasol (FIG. 13K) was similar to the use of nesting in animals treated with either Compound A or Compound B. Whilst there was some improvement in the use of nesting with deucravacitinib (FIG. 13J) compared to psoriasis induced animals treated with vehicle, the nesting score was about half that seen with Compound A.

TABLE 17G
Spleen Weight Ratio in IMI-Psoriasis Model.

| | Animal | Spleen | Animal | Spleen Weight |
|---|---|---|---|---|
| Group | Number# | Weight | Weight | Ratio |
| 1 (Vehicle + Vehicle) | 1 | 97.92 | 19.13 | 511.87 |
| | 2 | 101.29 | 19.17 | 528.38 |
| | 3 | 93.56 | 18.94 | 493.98 |
| | 4 | 85.36 | 18.09 | 471.86 |
| | 5 | 76.18 | 18.16 | 419.49 |
| | 6 | 93.72 | 18.89 | 496.14 |
| | 7 | 103.44 | 21.30 | 485.63 |
| | 8 | 92.07 | 19.51 | 471.91 |
| 2 (IMI + Vehicle) | 1 | 377.18 | 14.98 | 2517.89 |
| | 2 | 289.05 | 16.02 | 1804.31 |
| | 3 | 281.30 | 13.92 | 2020.83 |
| | 4 | 316.50 | 15.26 | 2074.05 |
| | 5 | 315.06 | 14.71 | 2141.81 |
| | 6 | 286.43 | 15.91 | 1800.31 |
| | 7 | 298.63 | 16.04 | 1861.78 |
| | 8 | 356.46 | 17.70 | 2013.90 |
| 3 (IMI + Compound A | 1 | 219.42 | 15.74 | 1394.03 |
| 1 mg/kg) | 2 | 202.00 | 17.76 | 1137.39 |
| | 3 | 212.50 | 16.69 | 1273.22 |
| | 4 | 233.20 | 17.05 | 1367.74 |
| | 5 | 220.14 | 17.07 | 1289.63 |
| | 6 | 227.26 | 17.34 | 1310.61 |
| | 7 | 202.76 | 17.23 | 1176.78 |
| | 8 | 213.15 | 16.41 | 1298.90 |
| 4 (IMI + Compound A | 1 | 199.90 | 18.44 | 1084.06 |
| 3 mg/kg) | 2 | 205.09 | 16.47 | 1245.23 |
| | 3 | 178.46 | 18.44 | 967.79 |
| | 4 | 185.94 | 15.82 | 1175.35 |
| | 5 | 214.46 | 16.95 | 1265.25 |
| | 6 | 180.80 | 16.91 | 1069.19 |
| | 7 | 187.63 | 16.13 | 1163.24 |
| | 8 | 151.76 | 16.47 | 921.43 |
| 5 (IMI + Compound A | 1 | 156.01 | 16.60 | 939.82 |
| 10 mg/kg) | 2 | 178.03 | 16.43 | 1083.57 |
| | 3 | 139.03 | 18.35 | 757.66 |
| | 4 | 157.80 | 19.39 | 813.82 |
| | 5 | 191.54 | 17.81 | 1075.46 |
| | 6 | 195.58 | 18.26 | 1071.08 |
| | 7 | 151.42 | 16.77 | 902.92 |
| | 8 | 172.86 | 15.96 | 1083.08 |
| 6 (IMI + Compound B | 1 | 255.35 | 16.34 | 1562.73 |
| 1 mg/kg) | 2 | 280.52 | 14.67 | 1912.20 |
| | 3 | 235.85 | 18.01 | 1309.55 |
| | 4 | 298.12 | 16.62 | 1793.74 |
| | 5 | 227.47 | 17.29 | 1315.62 |
| | 6 | 255.31 | 16.44 | 1552.98 |
| | 7 | 244.77 | 16.12 | 1518.42 |
| | 8 | 316.12 | 17.36 | 1820.97 |
| 7 (IMI + Compound B | 1 | 244.98 | 13.78 | 1777.79 |
| 3 mg/kg) | 2 | 212.34 | 16.88 | 1257.94 |
| | 3 | 270.73 | 15.93 | 1699.50 |
| | 4 | 183.55 | 18.05 | 1016.90 |
| | 5 | 232.29 | 17.29 | 1343.49 |
| | 6 | 224.28 | 17.08 | 1313.11 |
| | 7 | 206.85 | 15.94 | 1297.68 |
| | 8 | 220.98 | 17.29 | 1278.08 |
| 8 (IMI + Compound B | 1 | 184.80 | 18.01 | 1026.10 |
| 10 mg/kg) | 2 | 169.45 | 16.93 | 1000.89 |
| | 3 | 184.96 | 18.51 | 999.24 |
| | 4 | 119.62 | 17.31 | 691.05 |
| | 5 | 101.24 | 15.47 | 654.43 |
| | 6 | 156.25 | 16.86 | 926.75 |
| | 7 | 130.42 | 16.66 | 782.83 |
| | 8 | 159.58 | 17.96 | 888.53 |
| 9 (IMI + Apremilast | 1 | 180.97 | 18.27 | 990.53 |
| 3 mg/kg) | 2 | 286.06 | 18.85 | 1517.56 |
| | 3 | 424.79 | 18.94 | 2242.82 |
| | 4 | 208.70 | 18.85 | 1107.16 |
| | 5 | 207.33 | 15.48 | 1339.34 |
| | 6 | 227.51 | 18.37 | 1238.49 |
| | 7 | 223.09 | 18.62 | 1198.12 |
| | 8 | 221.44 | 18.57 | 1192.46 |
| 10 (IMI + | 1 | 195.00 | 18.03 | 1081.53 |
| Deucravacitinib | 2 | — | — | — |
| 3 mg/kg) | 3 | 187.27 | 16.39 | 1142.59 |
| | 4 | 171.30 | 16.00 | 1070.63 |
| | 5 | 177.57 | 16.29 | 1090.06 |
| | 6 | 171.11 | 17.17 | 996.56 |
| | 7 | 127.21 | 15.22 | 835.81 |
| | 8 | — | — | — |
| 11 (IMI + Clobetasol | 1 | 39.92 | 14.30 | 279.16 |
| 0.05%) | 2 | 30.38 | 14.75 | 205.97 |
| | 3 | 50.46 | 16.22 | 311.10 |
| | 4 | 44.29 | 15.41 | 287.41 |
| | 5 | 37.25 | 14.99 | 248.50 |
| | 6 | 36.61 | 13.32 | 274.85 |
| | 7 | 35.08 | 14.54 | 241.27 |
| | 8 | 34.69 | 14.22 | 243.95 |

# Each animal actually had a different number from 1 to 88, and the animal numbers are shown for simplicity as 1-8 for each group.

TABLE 17H
Use Enrichment in IMI-Psoriasis Model.

| | Cage | | | | | | |
|---|---|---|---|---|---|---|---|
| Group | Number# | D1 | D8 | D9 | D10 | D11 | D12 |
| 1 (Vehicle + Vehicle) | 1 | 4.0 | 4.0 | 1.0 | 3.5 | 3.5 | 3.0 |
| | 2 | 4.0 | 4.0 | 1.0 | 4.0 | 4.0 | 3.0 |
| | 3 | 4.0 | 4.0 | 2.5 | 4.0 | 4.0 | 3.5 |
| | 4 | 4.0 | 4.0 | 3.5 | 4.0 | 4.0 | 4.0 |
| 2 (IMI + Vehicle) | 1 | 4.0 | 1.0 | 0.0 | 0.5 | 1.0 | 1.0 |
| | 2 | 3.5 | 0.0 | 0.0 | 0.0 | 0.0 | 0.0 |
| | 3 | 4.0 | 2.0 | 0.5 | 1.0 | 1.0 | 1.0 |
| | 4 | 4.0 | 1.0 | 0.5 | 0.5 | 0.5 | 1.5 |
| 3 (IMI + Compound A 1 mg/kg) | 1 | 4.0 | 1.0 | 1.5 | 2.5 | 3.0 | 2.0 |
| | 2 | 4.0 | 1.5 | 1.5 | 3.5 | 4.0 | 2.5 |
| | 3 | 4.0 | 1.0 | 1.0 | 2.0 | 2.0 | 4.0 |
| | 4 | 4.0 | 1.0 | 0.0 | 2.0 | 4.0 | 4.0 |
| 4 (IMI + Compound A 3 mg/kg) | 1 | 4.0 | 1.0 | 1.0 | 2.0 | 3.0 | 3.0 |
| | 2 | 4.0 | 0.0 | 0.0 | 3.0 | 4.0 | 3.0 |
| | 3 | 4.0 | 1.0 | 0.0 | 2.0 | 3.0 | 3.0 |
| | 4 | 3.5 | 1.0 | 0.0 | 3.0 | 4.0 | 3.0 |
| 5 (IMI + Compound A 10 | 1 | 4.0 | 1.0 | 0.0 | 3.0 | 3.0 | 2.0 |
| mg/kg) | 2 | 4.0 | 1.5 | 3.0 | 4.0 | 4.0 | 4.0 |
| | 3 | 4.0 | 0.0 | 1.5 | 3.0 | 4.0 | 4.0 |
| | 4 | 4.0 | 1.5 | 0.0 | 3.0 | 4.0 | 3.0 |
| 6 (IMI + Compound B 1 mg/kg) | 1 | 4.0 | 1.0 | 1.5 | 2.5 | 3.0 | 1.5 |
| | 2 | 4.0 | 1.0 | 1.5 | 1.5 | 3.0 | 1.5 |
| | 3 | 4.0 | 1.5 | 2.0 | 3.0 | 3.5 | 1.0 |
| | 4 | 4.0 | 1.5 | 1.0 | 3.0 | 2.5 | 1.5 |
| 7 (IMI + Compound B 3 mg/kg) | 1 | 4.0 | 1.0 | 0.0 | 1.0 | 2.0 | 1.0 |
| | 2 | 4.0 | 1.5 | 2.5 | 3.5 | 4.0 | 3.0 |
| | 3 | 4.0 | 1.5 | 2.0 | 2.5 | 2.5 | 2.5 |
| | 4 | 4.0 | 0.5 | 0.5 | 1.5 | 2.0 | 0.0 |
| 8 (IMI + Compound B 10 | 1 | 4.0 | 1.0 | 2.0 | 3.0 | 4.0 | 3.5 |
| mg/kg) | 2 | 4.0 | 1.5 | 0.0 | 0.0 | 1.0 | 3.0 |
| | 3 | 4.0 | 0.0 | 0.0 | 0.5 | 1.5 | 1.0 |
| | 4 | 4.0 | 2.0 | 2.0 | 2.0 | 2.5 | 3.0 |
| 9 (IMI + Apremilast 3 mg/kg) | 1 | 4.0 | 1.0 | 0.0 | 2.5 | 3.5 | 3.5 |
| | 2 | 4.0 | 1.0 | 2.0 | 3.0 | 4.0 | 2.5 |
| | 3 | 4.0 | 1.0 | 0.5 | 3.0 | 3.0 | 3.0 |
| | 4 | 4.0 | 0.0 | 0.5 | 2.0 | 3.0 | 3.0 |
| 10 (IMI + Deucravacitinib 3 | 1 | 4.0 | 1.0 | 0.0 | 0.0 | 0.0 | 1.0 |
| mg/kg) | 2 | 4.0 | 1.0 | 0.0 | 0.0 | 0.0 | 2.0 |
| | 3 | 4.0 | 1.0 | 0.5 | 0.0 | 1.5 | 2.0 |
| | 4 | 4.0 | 0.0 | 0.0 | 0.0 | 0.0 | 0.0 |
| 11 (IMI + Clobetasol 0.05%) | 1 | 4.0 | 1.0 | 2.0 | 4.0 | 4.0 | 4.0 |
| | 2 | 4.0 | 1.5 | 1.5 | 3.5 | 4.0 | 3.5 |
| | 3 | 4.0 | 1.0 | 1.0 | 3.0 | 3.0 | 1.5 |
| | 4 | 4.0 | 0.5 | 1.0 | 3.0 | 4.0 | 2.5 |

| | Cage | | | | Mean | Mean |
|---|---|---|---|---|---|---|
| Group | Number | D13 | D14 | D15 | D1-D8 | D8-D15 |
| 1 (Vehicle + Vehicle) | 1 | 4.0 | 4.0 | 4.0 | 4.00 | 3.29 |
| | 2 | 4.0 | 4.0 | 4.0 | 4.00 | 3.43 |
| | 3 | 4.0 | 4.0 | 4.0 | 4.00 | 3.71 |
| | 4 | 4.0 | 4.0 | 4.0 | 4.00 | 3.93 |
| 2 (IMI + Vehicle) | 1 | 1.0 | 1.5 | 2.0 | 2.50 | 1.00 |
| | 2 | 0.0 | 0.0 | 0.0 | 1.75 | 0.00 |
| | 3 | 1.0 | 1.0 | 1.0 | 3.00 | 0.93 |
| | 4 | 1.5 | 1.5 | 1.5 | 2.50 | 1.07 |
| 3 (IMI + Compound A 1 mg/kg) | 1 | 4.0 | 4.0 | 4.0 | 2.50 | 3.00 |
| | 2 | 4.0 | 4.0 | 4.0 | 2.75 | 3.36 |
| | 3 | 4.0 | 4.0 | 4.0 | 2.50 | 3.00 |
| | 4 | 4.0 | 4.0 | 4.0 | 2.50 | 3.14 |
| 4 (IMI + Compound A 3 mg/kg) | 1 | 4.0 | 4.0 | 4.0 | 2.50 | 3.00 |
| | 2 | 4.0 | 4.0 | 4.0 | 2.00 | 3.14 |
| | 3 | 4.0 | 4.0 | 4.0 | 2.50 | 2.86 |
| | 4 | 4.0 | 4.0 | 4.0 | 2.25 | 3.14 |
| 5 (IMI + Compound A 10 mg/kg) | 1 | 4.0 | 4.0 | 4.0 | 2.50 | 2.86 |
| | 2 | 4.0 | 4.0 | 4.0 | 2.75 | 3.86 |
| | 3 | 4.0 | 4.0 | 4.0 | 2.00 | 3.50 |
| | 4 | 4.0 | 4.0 | 4.0 | 2.75 | 3.14 |
| 6 (IMI + Compound B 1 mg/kg) | 1 | 1.5 | 2.5 | 3.5 | 2.50 | 2.57 |
| | 2 | 2.0 | 2.0 | 3.0 | 2.50 | 2.07 |
| | 3 | 4.0 | 1.5 | 2.0 | 2.75 | 2.43 |
| | 4 | 4.0 | 1.5 | 1.0 | 2.75 | 2.14 |
| 7 (IMI + Compound B 3 mg/kg) | 1 | 2.0 | 3.0 | 3.5 | 2.50 | 1.79 |
| | 2 | 4.0 | 4.0 | 4.0 | 2.75 | 3.57 |
| | 3 | 2.5 | 3.5 | 4.0 | 2.75 | 2.79 |
| | 4 | 1.5 | 2.5 | 3.0 | 2.25 | 1.57 |
| 8 (IMI + Compound B 10 mg/kg) | 1 | 4.0 | 4.0 | 4.0 | 2.50 | 3.50 |
| | 2 | 4.0 | 4.0 | 4.0 | 2.75 | 2.29 |
| | 3 | 2.5 | 4.0 | 4.0 | 2.00 | 1.93 |
| | 4 | 4.0 | 4.0 | 4.0 | 3.00 | 3.07 |
| 9 (IMI + Apremilast 3 mg/kg) | 1 | 4.0 | 4.0 | 4.0 | 2.50 | 3.07 |
| | 2 | 4.0 | 4.0 | 4.0 | 2.50 | 3.36 |
| | 3 | 4.0 | 4.0 | 4.0 | 2.50 | 3.07 |
| | 4 | 4.0 | 4.0 | 4.0 | 2.00 | 2.93 |
| 10 (IMI + Deucravacitinib 3 mg/kg) | 1 | 2.0 | 3.0 | 4.0 | 2.50 | 1.43 |
| | 2 | 2.0 | 3.0 | 4.0 | 2.50 | 1.57 |
| | 3 | 2.5 | 3.5 | 4.0 | 2.50 | 2.00 |
| | 4 | 1.0 | 2.0 | 2.0 | 2.00 | 0.71 |
| 11 (IMI + Clobetasol 0.05%) | 1 | 4.0 | 4.0 | 4.0 | 2.50 | 3.71 |
| | 2 | 4.0 | 4.0 | 4.0 | 2.75 | 3.50 |
| | 3 | 3.5 | 4.0 | 4.0 | 2.50 | 2.86 |
| | 4 | 3.5 | 4.0 | 4.0 | 2.25 | 3.14 |

# Each cage actually had a different number from 1 to 44, and the cage numbers are shown for simplicity as 1−4 for each group. There were two animals per cage.

TABLE 17I
Behavioral Evaluation in IMI-Psoriasis Model.

| | | | | | | Distance |
|---|---|---|---|---|---|---|
| | Animal | Number | Number | Number Licking + | Number | Traveled |
| Group | Number# | Scratching | Licking | Scratching | Rearings | (m) |
| 1 (Vehicle + | 1 | 0 | 3 | 3 | 5 | 12 |
| Vehicle) | 2 | 2 | 10 | 12 | 81 | 15 |
| | 3 | 0 | 2 | 2 | 23 | 10 |
| | 4 | 0 | 3 | 3 | 37 | 13 |
| | 5 | 0 | 1 | 1 | 14 | 11 |
| | 6 | 0 | 4 | 4 | 13 | 5 |
| | 7 | 0 | 3 | 3 | 51 | 13 |
| | 8 | 0 | 2 | 2 | 50 | 13 |
| 2 (IMI + | 1 | 0 | 0 | 0 | 0 | 3 |
| Vehicle) | 2 | 0 | 6 | 6 | 31 | 18 |
| | 3 | 0 | 11 | 11 | 5 | 4 |
| | 4 | 1 | 5 | 6 | 2 | 6 |
| | 5 | 0 | 6 | 6 | 1 | 5 |
| | 6 | 0 | 1 | 1 | 0 | 16 |
| | 7 | 3 | 10 | 13 | 13 | 14 |
| | 8 | 1 | 2 | 3 | 0 | 7 |
| 3 (IMI + | 1 | 0 | 3 | 3 | 42 | 18 |
| Compound A 1 | 2 | 0 | 5 | 5 | 52 | 11 |
| mg/kg) | 3 | 7 | 1 | 8 | 50 | 14 |
| | 4 | 1 | 4 | 5 | 55 | 12 |
| | 5 | 0 | 3 | 3 | 35 | 14 |
| | 6 | 0 | 2 | 2 | 78 | 16 |
| | 7 | 0 | 0 | 0 | 46 | 16 |
| | 8 | 0 | 2 | 2 | 4 | 4 |
| 4 (IMI + | 1 | 0 | 3 | 3 | 41 | 12 |
| Compound A 3 | 2 | 0 | 5 | 5 | 43 | 12 |
| mg/kg) | 3 | 0 | 1 | 1 | 34 | 13 |
| | 4 | 0 | 1 | 1 | 24 | 8 |
| | 5 | 0 | 1 | 1 | 8 | 13 |
| | 6 | 0 | 4 | 4 | 34 | 12 |
| | 7 | 0 | 4 | 4 | 47 | 9 |
| | 8 | Freezing in the open field | | | | |
| 5 (IMI + | 1 | 1 | 2 | 3 | 6 | 4 |
| Compound A | 2 | 4 | 7 | 11 | 39 | 13 |
| 10 mg/kg) | 3 | 0 | 2 | 2 | 38 | 10 |
| | 4 | 2 | 2 | 4 | 46 | 13 |
| | 5 | 0 | 2 | 2 | 11 | 8 |
| | 6 | 1 | 1 | 2 | 21 | 7 |
| | 7 | 0 | 4 | 4 | 13 | 6 |
| | 8 | 0 | 5 | 5 | 55 | 11 |
| 6 (IMI + | 1 | 2 | 3 | 5 | 44 | 15 |
| Compound B 1 | 2 | 5 | 3 | 8 | 23 | 7 |
| mg/kg) | 3 | 0 | 3 | 3 | 47 | 11 |
| | 4 | 1 | 3 | 4 | 19 | 8 |
| | 5 | 3 | 2 | 5 | 29 | 9 |
| | 6 | 0 | 7 | 7 | 27 | 13 |
| | 7 | 2 | 4 | 6 | 87 | 14 |
| | 8 | 0 | 5 | 5 | 49 | 14 |
| 7 (IMI + | 1 | 0 | 8 | 8 | 27 | 17 |
| Compound B 3 | 2 | 2 | 9 | 11 | 38 | 9 |
| mg/kg) | 3 | 1 | 3 | 4 | 121 | 23 |
| | 4 | 0 | 3 | 3 | 47 | 12 |
| | 5 | 0 | 3 | 3 | 31 | 15 |
| | 6 | 2 | 3 | 5 | 60 | 11 |
| | 7 | Freezing in open field | | | | |
| | 8 | 5 | 8 | 13 | 57 | 13 |
| 8 (IMI + | 1 | Freezing in open field | | | | |
| Compound B | 2 | 0 | 6 | 6 | 44 | 15 |
| 10 mg/kg) | 3 | 1 | 4 | 5 | 44 | 14 |
| | 4 | 0 | 2 | 2 | 71 | 16 |
| | 5 | 1 | 2 | 3 | 6 | 7 |
| | 6 | 1 | 2 | 3 | 11 | 6 |
| | 7 | 0 | 2 | 2 | 1 | 7 |
| | 8 | 0 | 3 | 3 | 13 | 7 |
| 9 (IMI + | 1 | 1 | 4 | 5 | 20 | 10 |
| Apremilast 3 | 2 | 1 | 5 | 6 | 58 | 13 |
| mg/kg) | 3 | 0 | 1 | 1 | 21 | 8 |
| | 4 | 0 | 2 | 2 | 42 | 11 |
| | 5 | 0 | 2 | 2 | 81 | 17 |
| | 6 | 3 | 4 | 7 | 13 | 9 |
| | 7 | 0 | 3 | 3 | 60 | 15 |
| | 8 | 0 | 3 | 3 | 12 | 11 |
| 10 (IMI + | 1 | 1 | 1 | 2 | 31 | 14 |
| Deucravacitinib | 2 | — | — | — | — | — |
| 3 mg/kg) | 3 | Freezing in open field | | | | |
| | 4 | 0 | 2 | 2 | 22 | 9 |
| | 5 | 0 | 2 | 2 | 58 | 19 |
| | 6 | 3 | 7 | 10 | 68 | 12 |
| | 7 | 12 | 8 | 20 | 38 | 14 |
| | 8 | — | — | — | — | — |
| 11 (IMI + | 1 | 0 | 1 | 1 | 10 | 9 |
| Clobetasol | 2 | 0 | 2 | 2 | 6 | 5 |
| 0.05%) | 3 | 0 | 1 | 1 | 31 | 10 |
| | 4 | 0 | 2 | 2 | 6 | 9 |
| | 5 | 0 | 2 | 2 | 19 | 10 |
| | 6 | 0 | 2 | 2 | 4 | 10 |
| | 7 | 0 | 5 | 5 | 39 | 15 |
| | 8 | 0 | 5 | 5 | 25 | 16 |

# Each animal actually had a different number from 1 to 88, and the animal numbers are shown for simplicity as 1-8 for each group.

FIGS. 14A-14H provide representative images of dorsal depilated mice in the placebo group at day 8 (FIG. 14A) and day 15 (FIG. 14E), the Compound A (3 mg/kg) treatment group at day 8 (FIG. 14B) and day 15 (FIG. 14F), the deucravacitinib (3 mg/kg) treatment group at day 8 (FIG. 14C) and day 15 (FIG. 14G) and the Compound B treatment group at day 8 (FIG. 14D) and day 15 (FIG. 14H). As evident in FIGS. 14F and 14G, by day 15, animals treated with either deucravacitinib or Compound A were nearly entirely symptom free and have greatly improved PASI scores (FIGS. 9A-9D). Similarly, as evident in FIG. 14H animals treated with Compound B at 10 mg/ml were nearly entirely symptom free and had greatly improved PASI scores (FIGS. 9C, 9D).

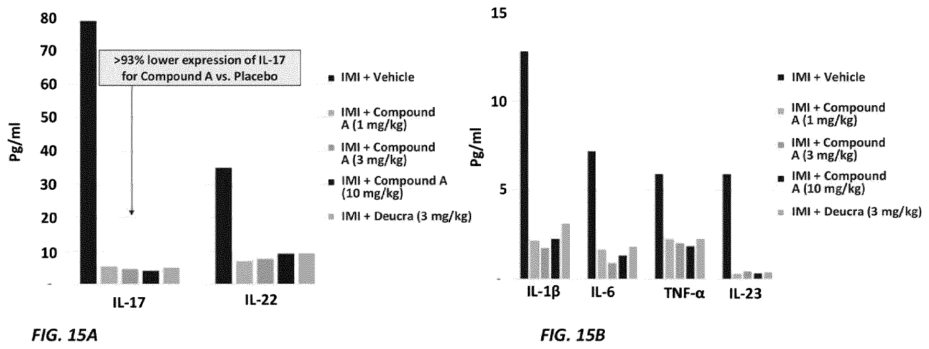

Compound A additionally was effective at dramatically reducing levels of key cytokines associated with the pathogenesis of psoriasis at all tested doses. Compound A administration also resulted in a marked reduction of other disease related Th17 and Th1 cytokines. FIG. 15A depicts levels of IL-17 and IL-22 in animals treated with Compound A, deucravacitinib, and vehicle after induction of psoriasis, and FIG. 15B illustrates levels of IL-1β, IL-6, TNF-α, and IL-23 in animals treated with compound A and vehicle after induction of psoriasis. Animals treated with Compound A exhibited a marked reduction of all cytokines assayed relative to vehicle-treated animals, and animals treated with Compound A exhibited levels of IL-17 that were about >93% lower than vehicle (placebo) treated animals (See FIG. 15A). Levels of cytokines in Compound A treated animals (all tested doses) were comparable to levels in animals treated with deucravacitinib for all tested cytokines.

The data for Compound A is evidence that it is capable of providing a very good efficacy on the treatment of psoriasis at the 3 tested doses, acting on the parameters monitored, with a level equivalent to those of Apremilast and Deucravacitinib.

The data for Compound B is evidence that it is capable of providing a very good efficacy on the treatment of psoriasis at the highest tested dose of 10 mg/kg by, acting on the parameters monitored, with a level equivalent to Apremilast and Deucravacitinib. A moderate efficacy was observed for the 2 lowest doses of 1.0 and 3.0 mg/kg bw.

Without being bound by any particular theory, the data from this study suggest that Compound B exhibits strong efficacy at the 10 mg/kg dose for all measured psoriasis parameters. Compound B (10 mg/kg) was similar in efficacy to Compound A (all three doses), Apremilast, and Decucravacitinib. Moderate efficacy was also observed at the 3 mg/kg and 1 mg/kg doses.

Without being bound by any particular theory, the data from this study indicate that Compound A (1 mg/kg, 3 mg/kg, and 10 mg/kg) and Compound B (10 mg/kg, and to a lesser extent, at doses of 1 mg/kg and 3 mg/kg) is comparable to deucravacitinib, clobetasol and apremilast in resolving induced dermal inflammation after 7 days of treatment and that Compound A (all tested doses) and Compound B (10 mg/kg dose, and to a lesser extent, at doses of 1 mg/kg and 3 mg/kg) exhibited strong efficacy in the treatment of psoriasis, and further acted on all monitored parameters, including allowing animals to significantly regain body weight, significantly reduce scores of the severity of psoriasis, restore animal behavior, significantly reduce the spleen weight ratio and significantly reduce inflammatory cytokine levels (i.e., IL-1β, IL-6, IL-17, TNF-α, IL-22, and IL-23), at levels at least similar to Apremilast and Deucravacitinib.

### Example 119: Collagen-Induced Arthritis in Animals Treated with a BET Inhibitor and Vehicle

The collagen-induced arthritis (CIA) model in rats was used to assess the efficacy of oral delivery of Compound A at three dosages (1 mg/kg, 3 mg/kg, and 10 mg/kg). Compound A suspensions were prepared according to the procedures set out in Experimental Method A. Evaluation of the effect of different dosages of Compound A compared to vehicle and controls in collagen-induced arthritis in rats was undertaken in accordance with the protocol set out in Experimental Method D.

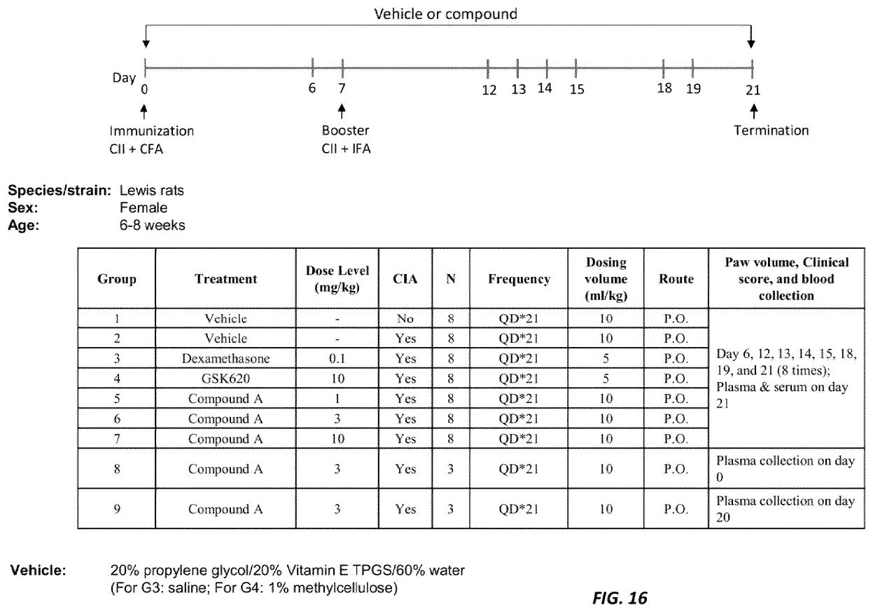

Arthritis was induced in Lewis rats by immunizing animals with the CFA+Collagen emulsion via subcutaneous injection at the base of the tail on day 0. Seven days after the first immunization, animals received a booster immunization with the IFA+Collagen emulsion via subcutaneous injection at the base of the tail. Animals not subjected to CIA did not receive the immunizations. A design of the CIA study is depicted in FIG. 16.

Starting on day 0, animals in group 1 and 2 were administered daily doses of the vehicle per os (PO) for 21 days. On day 0, animals in groups 3 and 4 were administered daily doses of dexamethasone and GSK620, respectively, PO for 21 days. Starting on day 0, animals in groups 4-9 were administered daily doses of Compound A (1 mg/kg, 3 mg/kg, or 10 mg/kg) for 21 days.

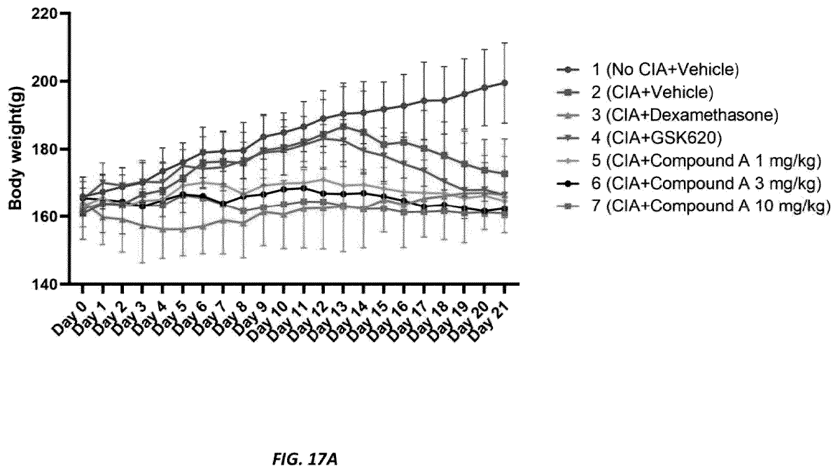

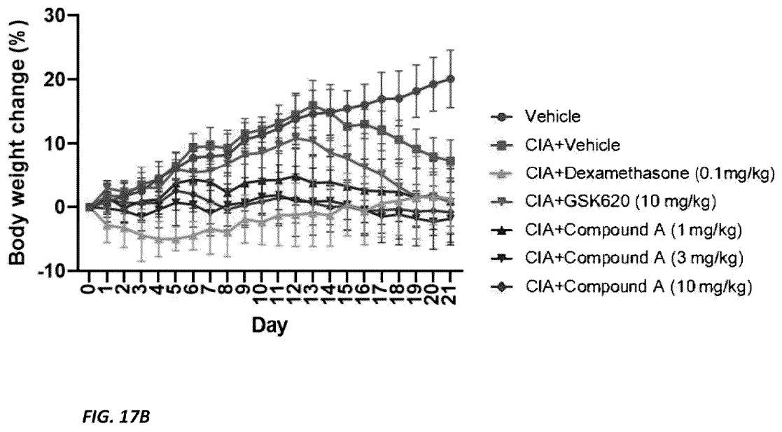

Animal body weight was measured daily over the course of the study (see Tables 18A-18D, FIGS. 17A-17B). CIA-induced animals receiving no treatment (group 2) exhibited a modest weight gain over the first 13 days followed by weight loss over the remaining study days. Despite the weight loss over the last seven days of the study, all animals in group 2 had gained weight when day 21 weights were compared to starting weights on day 0. In contrast, animals where CIA was not induced, and no treatment was administered (group 1) steadily gained weight over the course of the study and animal weight on day 21 was significantly increased relative to group 2 (p<0.01). Animals receiving dexamethasone did not exhibit weight gains over the first 13 days of the study, exhibiting significantly reduced weight (p<0.01) over days 4-7 and 9-17 relative to animals in group 2. Ultimately, only 4/8 animals receiving dexamethasone after induction of CIA exhibited a weight gain by the end of the study, whereas 2/8 animals lost weight, and 2/8 animals weighed the same at the end of the study as their starting weight. Animals receiving GSK620 (group 4) exhibited modest weight gains over the first 13 days of the study followed by weight loss, similar to animals in group 2. At day 21, 4/8 animals had gained weight after receiving daily administration of GSK620 following induction of CIA.

Animals receiving the low dose of Compound A (1 mg/kg, group 5) exhibited a weight gain over the first 12 days of the study, albeit at a lower rate relative to animals in groups 2 and 4, followed by a slight weight loss over the remaining days. On days 9-17, animal weight was significantly decreased (p<0.01) relative to animal weight in group 2. Animal weight was also significantly decreased (p<0.05) on days 18 and 19 relative to animal weight in group 2, but, by day 21, there was no significant difference in animal weight. In group 5, 7/8 animals exhibited increased weight at the end of the study. Animals in groups 6 and 7 (Compound A 3 mg/kg and 10 mg/kg, respectively) exhibited weight gains over the first 10-11 days of the study that were similar to animals in group 5 (Compound A 1 mg/kg), followed by slight weight loss over the remaining days of the study. Animals in group 6 exhibited weights that were significantly decreased relative to group 2 on days 7 and days 9-20 (p<0.01), and on days 5 and 19-21 (p<0.05). Only 2/8 animals in group 6 gained weight over the course of the study, whereas 6/8 animals all lost weight when comparing day 0 weights to day 21 weights. Similarly, animals in group 7 exhibited weights that were significantly decreased relative to group 2 on days 6-7 and days 9-20 (p<0.01), and on day 21 (p<0.05). In group 7, 4/8 animals gained weight over the course of the study, and the remaining 4/8 either lost weight or had no weight change when comparing day 0 weights to day 21 weights.

Comparing animal weights on day 21 to day 0, animals where CIA was not induced (group 1) experienced a 20.1% increase in body weight (Table 17D). In contrast, when CIA was induced but no treatment was provided (group 2), animals experienced a 7.4% increase in body weight. Animals in the dexamethasone group (group 3) exhibited a 1.3% increase in body weight. Animals in the GSK620 treatment group (group 4) and low-dose Compound A treatment group (group 5) exhibited 0.8% and 0.9% increases in body weight, respectively. Animals in the Compound A 3 mg/kg and 10 mg/kg groups experienced weight changes of approximately −1.7% and −0.75%, respectively at the end of the study.

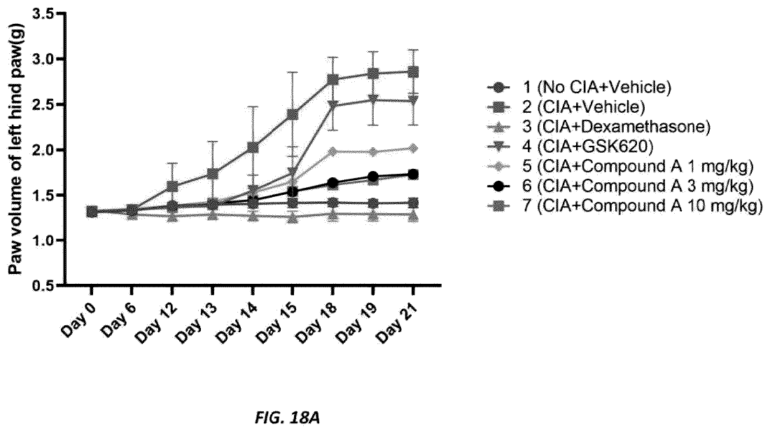

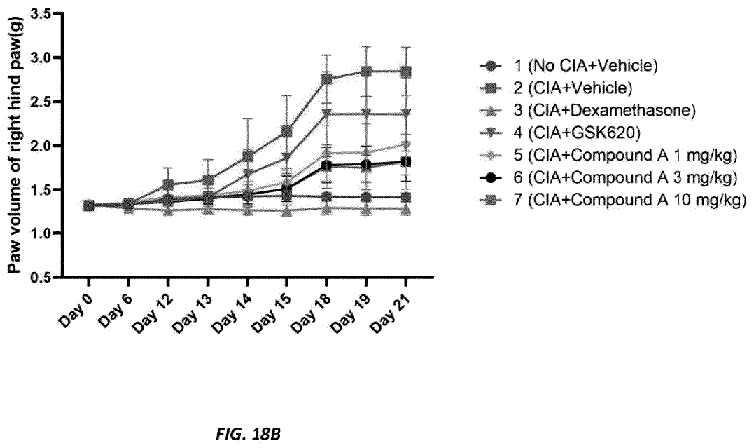

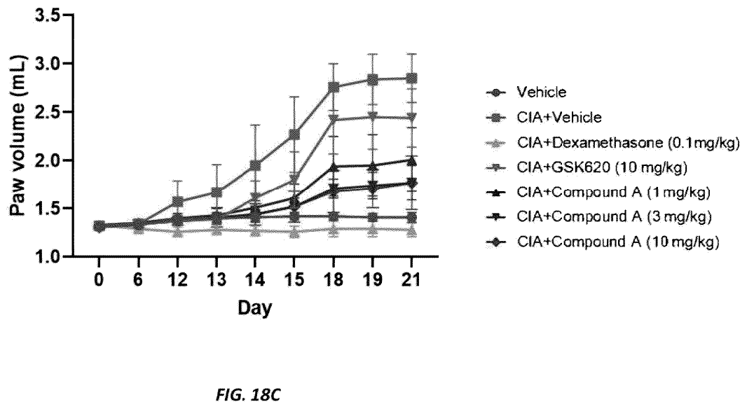

Animals where CIA was induced, and no treatment was provided exhibited a consistent increase in paw volume in both the right and left hind paw starting on day 6 and continuing throughout the study (Tables 19A-19D, FIGS. 18A-18C). There was generally no significant difference in paw volumes for the left hind paw relative to the right hind paw. Tables 19A and 19C show that paw volume in animals with CIA was significantly increased relative to animals where CIA was not induced on day 15 (both paws, p<0.05) and on days 18, 19, and 21 (both paws, p<0.01). Dexamethasone was effective at controlling paw volume, and animals receiving dexamethasone had paw volumes similar to uninduced animals, and these paw volumes were significantly decreased relative to animals in group 2 on days 14 (left paw, p<0.05), 15 (left paw, p<0.01; right paw, p<0.05), and 18, 19, and 21 (both paws, p<0.01). GSK620 (group 4) and Compound A 3 mg/mI (group 5) failed to prevent an increase in paw volume over the course of the study, with paw volumes similar to CIA induced animals receiving vehicle. Animals in group 5 (Compound A 1 mg/kg) exhibited an increase in paw volume of the study, but this increase in paw volume was significantly decreased relative to group 2 on days 18-20 in both hind limbs (p<0.01). Animals in group 6 (Compound A 3 mg/kg) also exhibited an increase in paw volume over the study, but this increase was significantly decreased relative to group 2 on day 15 (left hind limb, p<0.05) and on days 18-21 (both paws, p<0.01). The high dose of Compound A (10 mg/kg, group 7) performed similarly to the 3 mg/kg dose (group 6) in preventing an increase in paw volume, and paw volume was significantly decreased relative to group 2 on day 15 (left hind limb, p<0.05) and on days 18-21 (both paws, p<0.01). Paw volume increases were delayed in groups 5, 6, and 7, with appreciable increases not occurring until approximately day 18. Notably, the 10 mg/kg PO QD dose of Compound A resulted in about a 79% decrease in paw volume relative to CIA+Vehicle treated animals at the end of the study (Day 21). Without being bound by any theory, while vehicle-treated and GSK620-treated animals developed active arthritis as evidenced by increased paw volume at day 21, administration of Compound A ameliorated joint inflammation in a dose-dependent manner (i.e., the higher doses 3 mg/kg and 10 mg/kg demonstrated the lowest paw volumes).

Tables 19B and 19D show percent change in rat hind paw volume over the course of the study. As noted, there was no appreciable differences in paw volumes for the left hind paw relative to the right hind paw. Paw volumes remained constant for all groups on day 6, prior to the booster immunization. Animals in group 2 exhibited an approximately 20% increase in paw volume in both paws on day 12, and paw volume steadily increased until days 18-19, where paw volume plateaued at an increase of approximately 115% relative to day 0. Dexamethasone was effective at preventing any change in paw volume throughout the entire 21-day period. Animals receiving GSK620 exhibited modest increases in paw volume until day 14, when both paws had paw volumes that were approximately 20% greater than day 0, and paw volume continued to increase until it plateaued at day 18, with a paw volume that was between 75-90% increased relative to day 0. The low dose Compound A treatment (1 mg/kg, group 5) performed better than GSK620, with paw volume noticeably increasing and plateauing at around day 18, with a paw volume that was increased approximately 50% relative to day 0. The higher doses of Compound A were even more effective at controlling paw volume. While animals receiving the 3 mg/kg Compound A dose also exhibited an increase in paw volume around day 18, paw volume plateaued at an increased volume of approximately 40% relative to day 0. The highest dose of Compound A (10 mg/kg, group 7) proved most effective, with paw volume noticeably increasing around day 18-19, and plateauing at an increased volume of approximately 35% relative to day 0.

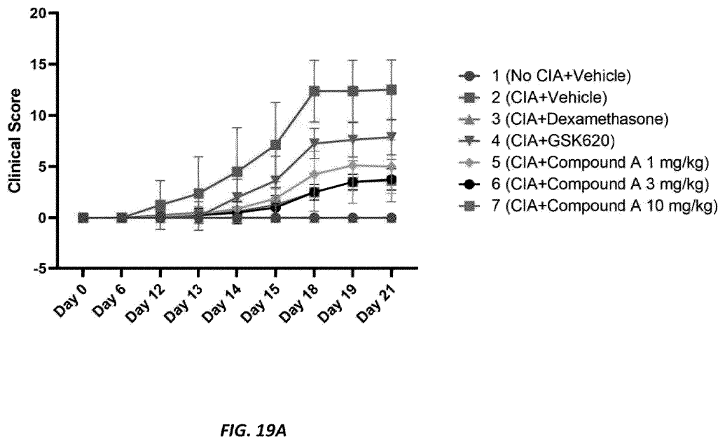

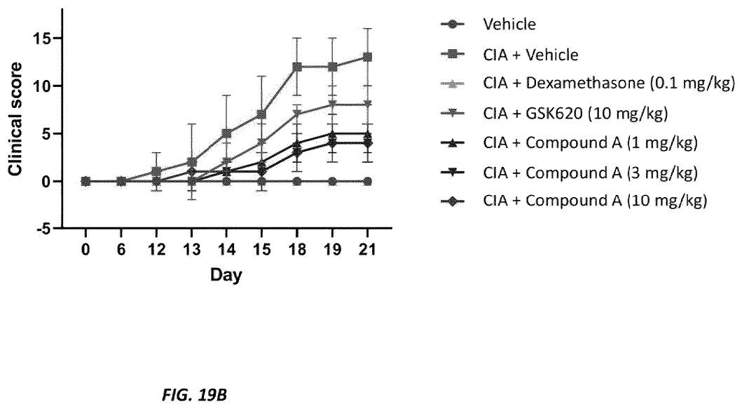

Tables 20A-20E and FIGS. 19A-19B show clinical scores and observations for animals in the different treatment groups. In FIGS. 19A-19B, Group 3 scored the same as Group 1, and data points for Group 3 may be hidden behind data points for Group 1. Similarly, Group 6 scored similarly to Group 7, and data points for Group 7 may be hidden behind those of Group 6. Clinical scores were recording according to Table 5B, outlined in Experimental Method D. No animals exhibited any signs of disease prior to day 12. On day 12, 2/8 animals in group 2 exhibited clinical symptoms, with one animal showing disease in both hind limbs, and the other showing disease in the left forelimb and both hind limbs. No animals in group 3 (dexamethasone) or group 4 (GSK620) showed any signs of disease on day 12. One animal in each of group 5 (Compound A 1 mg/kg), group 6 (Compound A 3 mg/kg), and group 7 (Compound A 10 mg/kg) exhibited signs of disease in either the right forelimb or the left forelimb on day 12. Nevertheless, Compound A at all three concentrations had lower clinical scores and—as evidenced by clinical scores at day 21—administration of Compound A ameliorated joint inflammation in a dose-dependent manner (i.e., with the higher doses of 3 mg/kg and 10 mg/kg demonstrating the lowest clinical scores). Notably, animals receiving the high dose of Compound A (10 mg/kg PO QD) exhibited about a 71% reduction in clinical score at the end of the study (Day 21) compared to vehicle-treated animals.

Animals in group 2 continued to exhibit symptoms, with 4/8 exhibiting symptoms on day 13, 6/8 exhibiting symptoms by day 14, and 8/8 animals exhibiting symptoms of disease by day 15. No animals in group 3 (dexamethasone) ever received a score indicating that they exhibited symptoms of disease. In group 4 (GSK620), on day 13 1/8 animals exhibited disease, and on day 14, 5/8 animals exhibited disease. By day 15, 7/8 animals exhibited disease, and on day 18 all animals were showing signs of disease. In group 5 (Compound A 1 mg/kg) 5/8 animals were showing disease by day 14, and 7/8 animals were exhibiting disease on day 18. In group 6 (Compound A 3 mg/kg) 4/8 animals were showing disease by day 15, and all animals were showing disease on day 18. In group 7 (Compound A 10 mg/kg) 4/8 animals were showing disease by day 15, and 7/8 animals were showing disease on day 18.

In group 2, on day 12, one animal received a score of 2 in both hind limbs and another animal received a score of 3 in one hind limb and a score of 2 in the other hind limb. By day 15, all animals in the group were given a score of at least 2 in one hind limb, and 4/8 animals received scores of 4 in one or both hind limbs, indicating the onset of severe arthritic disease. By day 18, all animals had received a score of 4 in each hind limb, and in some instances, in the fore limbs as well.

In group 4, on day 14, 5/8 animals received a clinical score of at least 2 in one or more hind limbs. By day 18, all animals received clinical scores of 3 or greater in one or more hind limbs. On day 21, 3/8 animals received a clinical score of 4 in each hind limb, and 5/8 animals received a score of 4 in at least one hind limb.

In group 5, on day 14, 1/8 animals received a clinical score of 2 in one hind limb. On day 15, 4/8 animals had clinical scores of at least 2 in one or more hind limbs, with one animal receiving a score of 2 for each hind limb. On days 18, 19 and 21, only 1/8 animals received clinical scores of 4 in one or both hind limbs. On days 19 and 20, 5/8 animals received a clinical score of 3 in one or both hind limbs. One animal received no clinical score over the course of the study. No animals received any clinical scores in the front limbs.

In group 6, on day 14, one animal received a score of 2 in one hind limb. On day 18, 5/8 animals received scores of 2 in one or both hind limbs. On day 19, 8/8 animals received clinical scores of 2 in one or both hind limbs, and 2/8 animals received a clinical score of 3 on one hind limb. Clinical scores on day 21 were similar, but with 3/8 animals receiving a clinical score of 3 on one hind limb. Similar to group 5, no animals received a clinical score on a front limb.

In group 7, on day 14, one animal received a score of 2 on one hind limb. On day 15, 2/8 animals received a score of 2 on one or both hind limbs. On day 18, 4/8 animals received scores of 2 or 3 on both hind limbs. On days 19 and 21, 7/8 animals received a score of 2 or 3 on both hind limbs. One animal received no clinical score over the course of the study.

Table 20D shows the average overall clinical score for animals in each treatment group. As expected, animals where CIA was not induced (group 1) showed no signs of disease over the 21-day period. In contrast, in group 2, where CIA was induced and no treatment was provided (group 2), animals exhibited an increase in clinical scores starting on day 12, and plateauing with an average clinical score of 13 around day 18. As previously noted, dexamethasone treated animals (group 3) never exhibited any signs of disease. Animals receiving GSK620 (group 4) exhibited an increase in clinical scores starting at day 14, with scores plateauing at an average clinical score of 8 around day 18.

On day 18, animals in group 4 exhibited an average clinical score that was significantly decreased (p<0.05) relative to group 2. Animals in group 5 exhibited a clinical score that progressed similar to other treatment groups, plateauing at an average score of 5 around day 18-19. In particular, animals in group 5 exhibited a significantly reduced (p<0.01) overall clinical score relative to group 2 on days 18, 19, and 21. Animals in groups 6 and 7 had average clinical scores that plateaued at 4, around day 18-19. Animals in groups 6 and 7 also exhibited significantly reduced (p<0.01) overall clinical scores relative to group 2 on days 18, 19, and 21.

Without being bound by any particular theory, the three different doses of Compound A (1 mg/kg, group 5; 3 mg/kg, group 6; and 10 mg/kg, group 7) appeared to exhibit a dose-dependent effect in the amelioration of clinical symptoms associated with CIA, with the higher doses of Compound A providing the greatest reduction in clinical symptoms.

Animals where CIA was not induced (group 1), as well as animals receiving vehicle (group 2), dexamethasone (group 3), and GSK620 (group 4) did not have diarrhea over the 21-day period (Table 20E). Two different animals receiving 1 mg/kg of Compound A (group 5) had diarrhea for a single day over the 21-day period. Four different animals receiving 3 mg/kg of Compound A (group 6) had diarrhea over the 21-day period, with one animal having diarrhea on successive days, and another animal having diarrhea on day 14 and day 21. Of the animals in group 7, four animals had diarrhea. One animal had diarrhea on days 12-18, another animal on days 13, 14, and 16, and another animal on days 14-16.

Without being bound by any particular theory, Compound A appeared to function in a dose-dependent manner to limit and/or reduce the severity of CIA-related symptoms in animals. The higher dosages of 3 mg/kg and 10 mg/kg proved most effective as shown by clinical scoring of CIA-related symptoms but were slightly less tolerated by animals as shown by the onset of diarrhea. As indicated by the dog PK studies #1 and #2 provided earlier (comparing a propylene glycol-based formulation and a HPβCD-based formulation), the slight reduction in tolerance may be, without being bound by any theory, due to the propylene glycol formulation rather than Compound A.

Table 21 shows a comparison of the percent inhibition in clinical score and paw volume when animals were treated with either Compound A (1 mg/kg or 3 mg/kg) or GSK620 (10 mg/kg). Compound A inhibited both increases in paw volume and clinical score that were notably higher than GSK620. For example, animals receiving either dose of Compound A exhibited percent inhibitions in increase in paw volume that were more than twice the percent inhibition measured when animals received GSK620. Similar trends were observed in clinical scores, where the percent inhibition in clinical score was nearly twice as high in Compound A treated animals (1 mg/kg and 3 mg/kg) than in animals receiving GSK620.

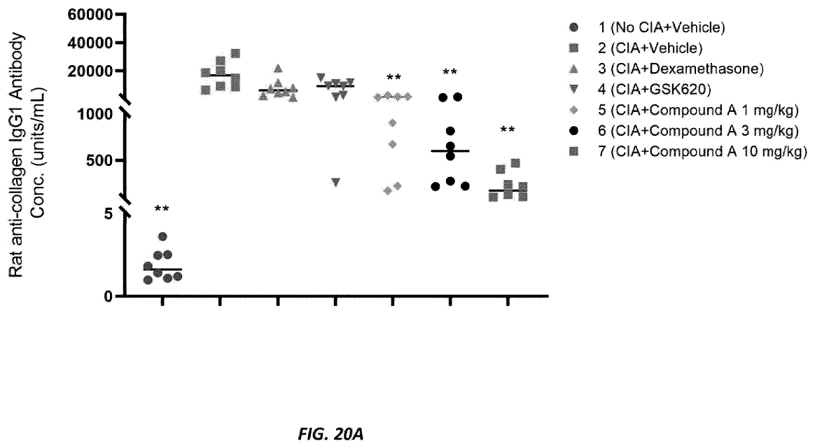

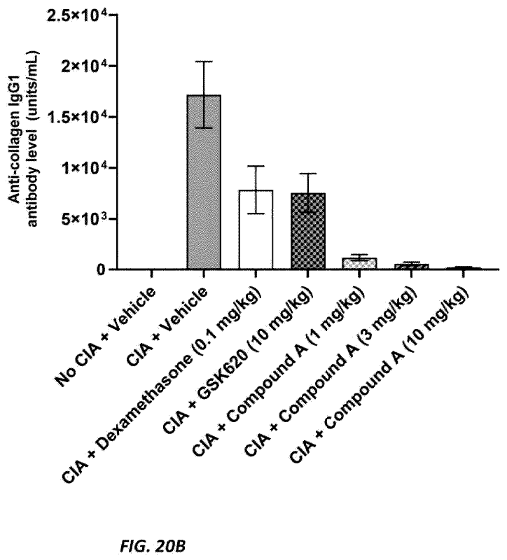

Tables 22A and 22B and FIGS. 20A-20B illustrate levels of rat anti-collagen IgG1 antibodies in animals at day 21 of the study. Animals receiving Compound A (1 mg/kg, 3 mg/kg, and 10 mg/kg) all exhibited significantly decreased levels of rat anti-collagen IgG1 antibodies relative to animals where CIA was induced, and no treatment was provided (p<0.01). Specifically, animals receiving the high dose of Compound A (10 mg/kg), exhibited levels of rat anti-collagen IgG1 antibodies that were >98% lower than the level of rat anti-collagen IgG1 antibodies in vehicle treated animals. Without being bound by any particular theory, Compound A appeared to reduce serum levels of rat anti-collagen IgG1 antibodies in a dose-dependent manner.

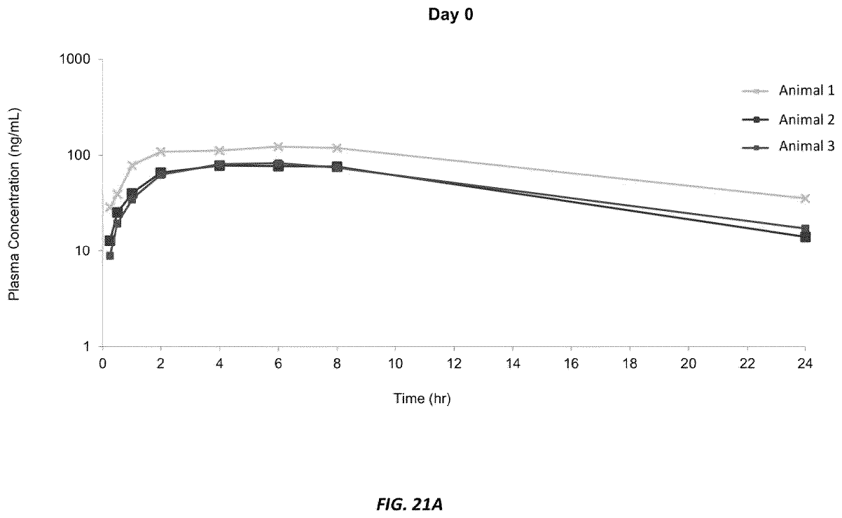

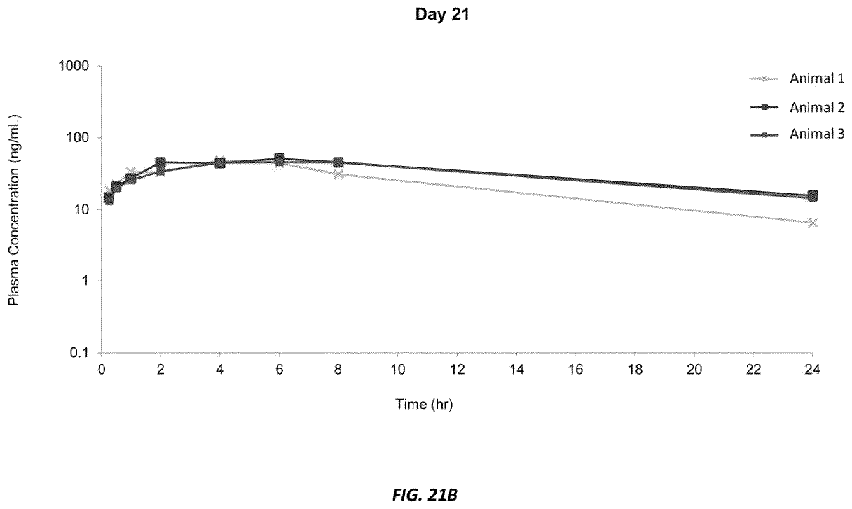

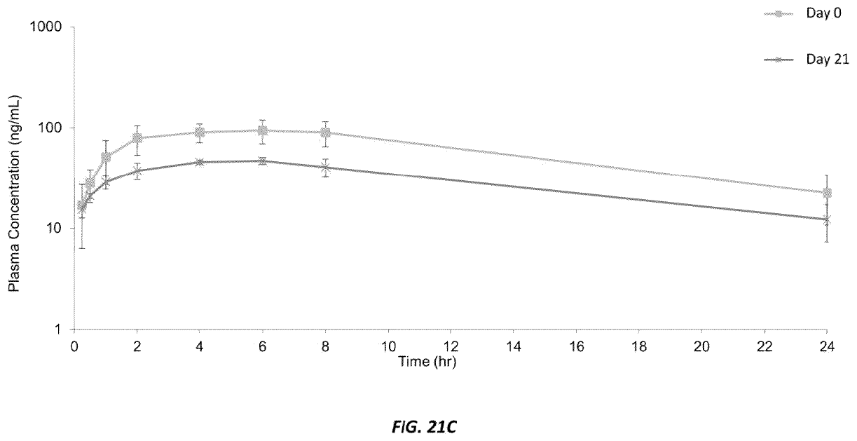

Tables 23A-23J and FIGS. 21A-21C depict the pharmacokinetic profile of Compound A after oral administration in Lewis rats. Without being bound by any particular theory, day 21 plasma concentration of Compound A appeared to increase in a dose-dependent manner. Plasma concentration of Compound A increased for approximately the first four hours after oral administration, and then plasma levels slowly declined over the remain time points. The pharmacokinetic profile indicates that Compound A may be therapeutically effective with a single daily unit dose. The free EC50 for GSK620 was 771 ng/mL, whereas the free EC50 for Compound A was 8 ng/mL. A plasma concentration of compound above the free EC50 for a significant period of time following an oral dose is an indicator of an effective treatment window. Also, having a low free EC50 can allow for different unit treatment doses that can be therapeutically effective, including a low unit dose of compound.

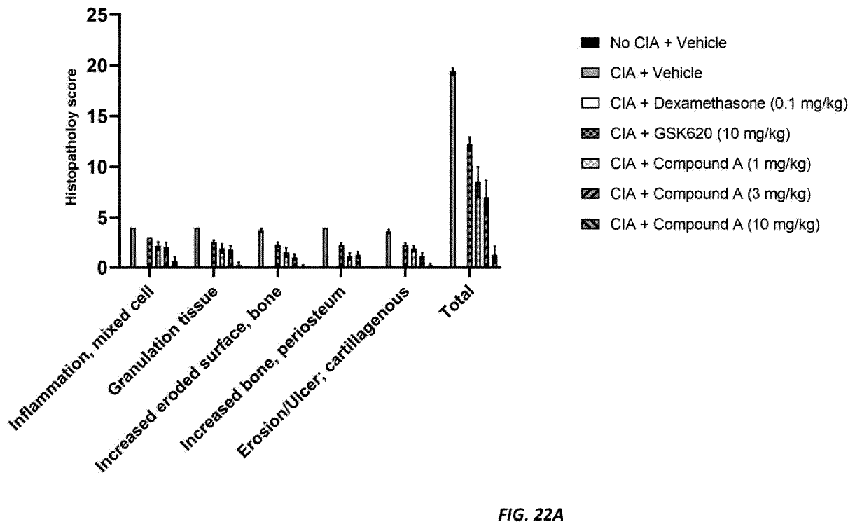

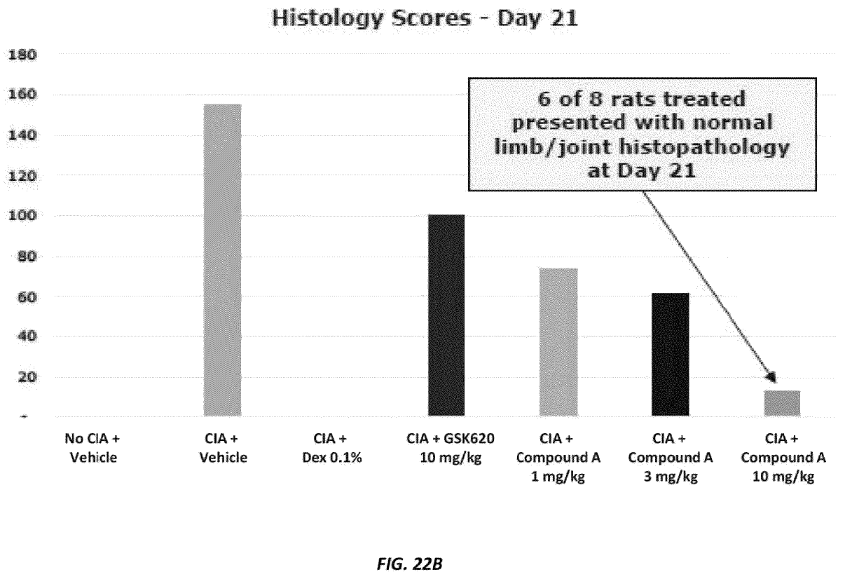

Results from the histopathological analysis are outlined in Table 24. Mean histopathologic scores for tissue samples from animals in Groups 1-6 are depicted in FIG. 22A, and day 21 histology scores for all animals are illustrated in FIG. 22B. A dose-dependent reduction in the severity of arthritic histopathology was observed for Compound A-treated animals that was statistically superior to vehicle-treated and GSK620-treated animals at all tested doses. At the conclusion of the study on day 21, 6/8 animals treated with Compound A (10 mg/kg) exhibited normal limb/joint pathology, demonstrating a significant improvement over vehicle-treated animals.

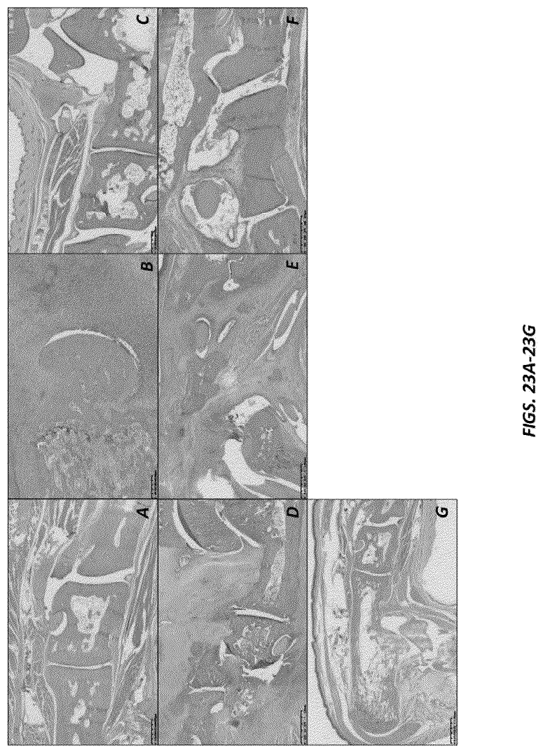

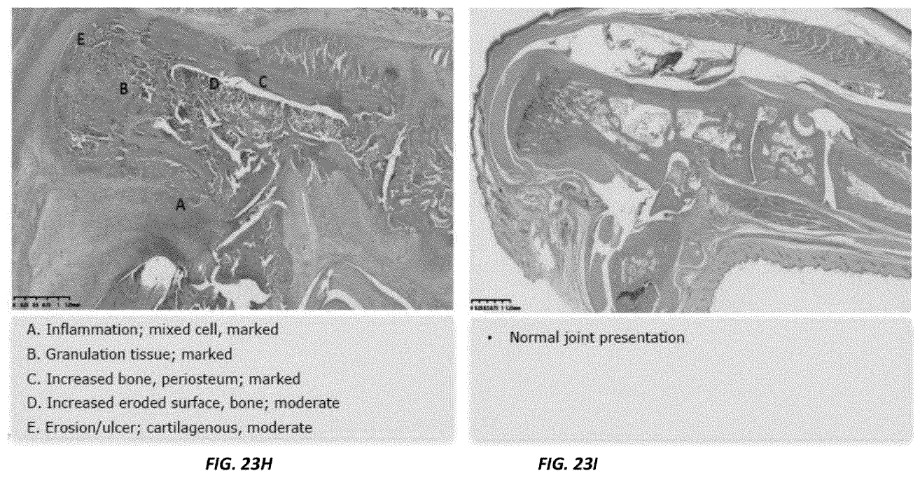

Representative images depicting different grades of histological measures are provided in FIGS. 23A-23G. FIG. 23A shows a representative sample from Group 1 (No CIA) depicting normal tissue having no histopathologic observations. FIG. 23B shows a representative sample from Group 2 (CIA+Vehicle) depicting marked mixed cell inflammation, marked granulation tissue, moderate increased eroded surface of bone, marked increased periosteal bone, and moderate erosion/ulcer in articular cartilage. FIG. 23C shows a representative sample from Group 3 (CIA+Dexamethasone) depicting tissue with no histopathological observations noted. FIG. 23D shows a representative sample from Group 4 (CIA+GSK620) depicting moderate mixed cell inflammation, mild granulation tissue, mild increased eroded surface of bone, mild increased periosteal bone, and mild erosion/ulcer in articular cartilage. FIG. 23E shows a representative sample from Group 5 (CIA+Compound A, 1 mg/kg) depicting moderate mixed cell inflammation, mild granulation tissue, mild increased eroded surface of bone, mild increased periosteal bone, and mild erosion/ulcer in articular cartilage. FIG. 23F shows a representative sample from Group 6 (CIA+Compound A, 3 mg/kg) depicting moderate mixed cell inflammation, mild granulation tissue, mild increased eroded surface of bone, mild increased periosteal bone, and mild erosion/ulcer in articular cartilage. FIG. 23G shows a representative sample from Group 7 (CIA+Compound A, 10 mg/kg), depicting tissue with no histopathological observations noted.

No pathologic observations were noted after microscopic evaluation for samples from animals in the vehicle-treated (sham) group (Group 1).

Pathologic observations were noted after microscopic evaluation for all samples from animals in the CIA-induced group (Group 2). Observations included marked mixed cell inflammation, marked granulation tissue (pannus), marked increased eroded surface of bone (bone resorption), moderate to marked increased periosteal bone (periosteal bone formation), and moderate to marked erosion/ulcer in articular cartilage.

No pathologic observations were noted after microscopic evaluation for samples from animals in the dexamethasone treatment group (Group 3).

Pathologic observations were noted after microscopic evaluation for all samples from animals in the GSK620 treatment group (Group 4), albeit at a lower level relative to samples from animals in the CIA-induced group (Group 2). Observations included moderate mixed cell inflammation, mild to moderate granulation tissue (pannus), minimal to moderate increased eroded surface of bone (bone resorption), mild to moderate increased periosteal bone (periosteal bone formation), and mild to moderate erosion/ulcer in the articular cartilage.

Pathologic observations were noted after microscopic evaluation of samples from animals in the Compound A treatment groups (Groups 5-7), albeit at lower levels relative to samples from animals in the CIA-induced group (Group 2). Mixed cell inflammation, granulation tissue (pannus), increased eroded surface of bone (bone resorption), increased periosteal bone (periosteal bone formation), and erosion/ulcer in the articular cartilage were all decreased in severity, extent, and distribution (generally focal to multifocal and/or involved less soft tissue, bone, and/or cartilage).

One out of eight samples from animals receiving Compound A (1 mg/kg; Group 5) had no pathologic observations. Seven out of eight samples from animals exhibited moderate to marked mixed cell inflammation, mild to moderate granulation tissue (pannus), mild to moderate increased eroded surface of bone (bone resorption), mild to moderate increased periosteal bone (periosteal bone formation), and mild to moderate erosion/ulcer in the articular cartilage.

Two out of eight samples from animals receiving Compound A (3 mg/kg; Group 6) had no pathologic observations. Six out of eight samples from animals exhibited moderate mixed cell inflammation, mild to moderate granulation tissue (pannus), mild increased eroded surface of bone (bone resorption), mild increased periosteal bone (periosteal bone formation), and mild erosion/ulcer in the articular cartilage.

Six out of eight samples from animals receiving Compound A (10 mg/kg; Group 7) had no pathologic observations. Two out of eight samples from animals exhibited mild to moderate mixed cell inflammation, mild granulation tissue (pannus), minimal increased periosteal bone (periosteal bone formation), and mild erosion in the articular cartilage.

FIG. 23H shows a representative image of a CIA vehicle-treated animal relative to an animal treated with Compound A (10 mg·kg PO QD) (FIG. 23I), where normal joint pathology is presented.

Without being bound to a particular theory, Compound A resulted in a significant dose-dependent decrease in the incidence and severity of collagen-induced arthritis as evidenced by histopathological analysis.

In sum, without being bound by any theory it is notable amongst other things that the CIA animal model demonstrated that Compound A provides a) potent anti-inflammatory activity and was observed to be superior (statistically) at 1, 3, and 10 mg/kg to the CIA placebo, and was also superior (statistically) to CIA+10 mg/kg GSK620 at all three doses, and by way of illustration, a major reduction in both clinical score and paw volume of about >70% with 10 mg/kg P OD was recorded compared to CIA+ placebo at Day 21; b) a dose dependent reduction in histopathology that was superior (statistically) at all three doses to the CIA+ placebo and to CIA+10 mg/kg GSK620; and c) near complete inhibition of IgG1 at all three doses, with e.g., IgG1 expression being reduced by >98% with 10 mg/kg Compound A POOD compared to CIA+ placebo.

Example 119 Tables—Part A: Body Weight, Paw Volumes, Clinical Scores and Inhibition

TABLE 18A
Animal body weight (g)

| | Animal | | | | | | | | |
|---|---|---|---|---|---|---|---|---|---|
| Group | # | Day 0 | Day 1 | Day 2 | Day 3 | Day 4 | Day 5 | Day 6 | Day 7 |
| 1 | 1 | 165 | 165 | 167 | 170 | 173 | 173 | 178 | 183 |
| (No CIA + | 2 | 156 | 161 | 162 | 166 | 170 | 173 | 173 | 172 |
| Vehicle) | 3 | 170 | 172 | 175 | 175 | 183 | 183 | 185 | 189 |
| | 4 | 168 | 165 | 168 | 169 | 171 | 179 | 179 | 179 |
| | 5 | 175 | 177 | 177 | 180 | 185 | 185 | 194 | 185 |
| | 6 | 166 | 165 | 163 | 167 | 170 | 176 | 177 | 178 |
| | 7 | 167 | 170 | 173 | 173 | 171 | 171 | 176 | 176 |
| | 8 | 161 | 164 | 166 | 161 | 165 | 169 | 170 | 173 |
| 2 | 1 | 165 | 169 | 171 | 176 | 172 | 180 | 184 | 183 |
| (CIA + Vehicle) | 2 | 163 | 171 | 170 | 170 | 173 | 175 | 180 | 183 |
| | 3 | 164 | 163 | 166 | 164 | 170 | 173 | 178 | 174 |
| | 4 | 175 | 179 | 177 | 185 | 182 | 189 | 187 | 190 |
| | 5 | 152 | 156 | 155 | 158 | 161 | 162 | 170 | 172 |
| | 6 | 160 | 160 | 157 | 160 | 161 | 170 | 172 | 173 |
| | 7 | 153 | 155 | 152 | 155 | 158 | 156 | 164 | 162 |
| | 8 | 155 | 157 | 161 | 165 | 166 | 167 | 173 | 174 |
| 3 | 1 | 151 | 152 | 150 | 148 | 147 | 147 | 147 | 150 |
| (CIA + | 2 | 162 | 150 | 151 | 144 | 147 | 149 | 152 | 153 |
| Dexamethasone) | 3 | 174 | 171 | 176 | 175 | 170 | 165 | 171 | 178 |
| | 4 | 169 | 165 | 166 | 164 | 160 | 164 | 161 | 162 |
| | 5 | 166 | 164 | 161 | 162 | 156 | 158 | 155 | 160 |
| | 6 | 161 | 151 | 148 | 145 | 148 | 150 | 149 | 145 |
| | 7 | 172 | 167 | 165 | 164 | 165 | 166 | 165 | 162 |
| | 8 | 160 | 158 | 156 | 156 | 157 | 151 | 157 | 161 |
| 4 | 1 | 157 | 160 | 162 | 163 | 159 | 167 | 169 | 170 |
| (CIA + GSK620) | 2 | 168 | 173 | 167 | 167 | 172 | 177 | 171 | 175 |
| | 3 | 165 | 171 | 168 | 168 | 172 | 172 | 172 | 170 |
| | 4 | 162 | 165 | 171 | 173 | 173 | 177 | 171 | 171 |
| | 5 | 170 | 175 | 173 | 172 | 171 | 180 | 177 | 180 |
| | 6 | 161 | 167 | 164 | 166 | 165 | 171 | 171 | 172 |
| | 7 | 173 | 178 | 179 | 181 | 179 | 181 | 186 | 183 |
| | 8 | 166 | 172 | 170 | 173 | 171 | 176 | 177 | 176 |
| 5 | 1 | 163 | 167 | 166 | 165 | 168 | 170 | 170 | 171 |
| (CIA + | 2 | 165 | 169 | 169 | 166 | 166 | 178 | 180 | 182 |
| Compound A 1 | 3 | 164 | 158 | 159 | 159 | 159 | 165 | 157 | 157 |
| mg/kg) | 4 | 162 | 169 | 167 | 169 | 166 | 172 | 176 | 171 |
| | 5 | 152 | 155 | 151 | 156 | 157 | 156 | 161 | 162 |
| | 6 | 169 | 170 | 166 | 164 | 170 | 171 | 171 | 170 |
| | 7 | 167 | 167 | 165 | 167 | 166 | 171 | 171 | 174 |
| | 8 | 163 | 170 | 161 | 171 | 169 | 171 | 175 | 169 |
| 6 | 1 | 168 | 163 | 162 | 160 | 167 | 166 | 163 | 157 |
| (CIA + | 2 | 171 | 172 | 171 | 168 | 173 | 177 | 174 | 172 |
| Compound A 3 | 3 | 175 | 175 | 173 | 170 | 175 | 178 | 176 | 173 |
| mg/kg) | 4 | 167 | 161 | 162 | 162 | 159 | 160 | 160 | 162 |
| | 5 | 167 | 166 | 166 | 164 | 165 | 163 | 164 | 163 |
| | 6 | 163 | 166 | 165 | 162 | 159 | 168 | 167 | 164 |
| | 7 | 157 | 160 | 159 | 160 | 165 | 167 | 168 | 166 |
| | 8 | 156 | 158 | 158 | 158 | 156 | 154 | 157 | 154 |
| 7 | 1 | 173 | 165 | 170 | 169 | 169 | 167 | 168 | 164 |
| (CIA + | 2 | 164 | 168 | 167 | 167 | 163 | 172 | 167 | 168 |
| Compound A 10 | 3 | 167 | 171 | 170 | 171 | 169 | 172 | 172 | 173 |
| mg/kg) | 4 | 159 | 163 | 159 | 158 | 160 | 161 | 165 | 160 |
| | 5 | 160 | 164 | 164 | 170 | 168 | 170 | 170 | 168 |
| | 6 | 156 | 154 | 154 | 154 | 155 | 160 | 155 | 153 |
| | 7 | 161 | 165 | 161 | 157 | 161 | 163 | 165 | 159 |
| | 8 | 158 | 162 | 162 | 160 | 162 | 166 | 162 | 164 |

| | Animal | | | | | | | |
|---|---|---|---|---|---|---|---|---|
| Group | # | Day 8 | Day 9 | Day 10 | Day 11 | Day 12 | Day 13 | Day 14 |
| 1 | 1 | 178 | 180 | 184 | 187 | 192 | 192 | 191 |
| (No CIA + | 2 | 175 | 181 | 180 | 177 | 187 | 185 | 185 |
| Vehicle) | 3 | 192 | 190 | 192 | 199 | 200 | 204 | 204 |
| | 4 | 181 | 186 | 187 | 188 | 183 | 189 | 191 |
| | 5 | 192 | 196 | 194 | 195 | 202 | 204 | 205 |
| | 6 | 171 | 180 | 182 | 183 | 183 | 183 | 183 |
| | 7 | 176 | 180 | 183 | 184 | 185 | 185 | 185 |
| | 8 | 172 | 176 | 177 | 180 | 180 | 181 | 182 |
| 2 | 1 | 180 | 190 | 188 | 190 | 192 | 200 | 197 |
| (CIA + Vehicle) | 2 | 182 | 184 | 183 | 189 | 190 | 193 | 190 |
| | 3 | 174 | 182 | 183 | 180 | 187 | 192 | 191 |
| | 4 | 193 | 197 | 195 | 193 | 202 | 203 | 203 |
| | 5 | 172 | 172 | 174 | 174 | 177 | 180 | 174 |
| | 6 | 170 | 172 | 173 | 176 | 173 | 174 | 168 |
| | 7 | 163 | 169 | 173 | 175 | 173 | 177 | 180 |
| | 8 | 173 | 171 | 175 | 180 | 181 | 174 | 176 |
| 3 | 1 | 147 | 153 | 154 | 155 | 153 | 151 | 157 |
| (CIA+ | 2 | 143 | 145 | 152 | 150 | 152 | 150 | 147 |
| Dexamethasone) | 3 | 174 | 176 | 176 | 183 | 185 | 186 | 181 |
| | 4 | 160 | 166 | 164 | 167 | 165 | 162 | 166 |
| | 5 | 161 | 162 | 159 | 157 | 162 | 167 | 163 |
| | 6 | 152 | 158 | 150 | 150 | 147 | 148 | 150 |
| | 7 | 167 | 172 | 175 | 173 | 170 | 176 | 175 |
| | 8 | 160 | 159 | 155 | 164 | 166 | 164 | 160 |
| 4 | 1 | 168 | 171 | 165 | 174 | 177 | 176 | 170 |
| (CIA + GSK620) | 2 | 176 | 177 | 177 | 182 | 185 | 182 | 176 |
| | 3 | 178 | 181 | 181 | 181 | 184 | 182 | 180 |
| | 4 | 177 | 178 | 180 | 178 | 179 | 179 | 177 |
| | 5 | 184 | 183 | 189 | 189 | 190 | 194 | 191 |
| | 6 | 171 | 174 | 178 | 177 | 181 | 175 | 176 |
| | 7 | 183 | 186 | 186 | 186 | 186 | 185 | 184 |
| | 8 | 176 | 182 | 180 | 183 | 183 | 187 | 182 |
| 5 | 1 | 166 | 169 | 170 | 171 | 174 | 166 | 172 |
| (CIA + | 2 | 172 | 180 | 178 | 180 | 182 | 180 | 178 |
| Compound A 1 | 3 | 157 | 156 | 155 | 157 | 158 | 158 | 159 |
| mg/kg) | 4 | 170 | 175 | 178 | 178 | 176 | 179 | 179 |
| | 5 | 161 | 162 | 166 | 162 | 162 | 160 | 155 |
| | 6 | 172 | 173 | 170 | 170 | 174 | 174 | 175 |
| | 7 | 171 | 170 | 176 | 175 | 177 | 172 | 170 |
| | 8 | 165 | 169 | 166 | 167 | 165 | 165 | 168 |
| 6 | 1 | 160 | 162 | 162 | 160 | 158 | 162 | 163 |
| (CIA + | 2 | 174 | 171 | 172 | 172 | 176 | 175 | 177 |
| Compound A 3 | 3 | 173 | 178 | 177 | 178 | 170 | 166 | 170 |
| mg/kg) | 4 | 169 | 169 | 170 | 172 | 167 | 173 | 175 |
| | 5 | 163 | 160 | 168 | 160 | 158 | 157 | 156 |
| | 6 | 165 | 163 | 167 | 169 | 169 | 168 | 167 |
| | 7 | 167 | 170 | 167 | 172 | 172 | 171 | 170 |
| | 8 | 157 | 160 | 162 | 165 | 165 | 162 | 158 |
| 7 | 1 | 162 | 164 | 165 | 164 | 163 | 163 | 164 |
| (CIA + | 2 | 164 | 165 | 165 | 173 | 173 | 170 | 169 |
| Compound A 10 | 3 | 165 | 168 | 172 | 170 | 170 | 172 | 170 |
| mg/kg) | 4 | 160 | 160 | 161 | 162 | 160 | 157 | 154 |
| | 5 | 164 | 165 | 167 | 167 | 168 | 162 | 161 |
| | 6 | 155 | 152 | 153 | 152 | 153 | 156 | 157 |
| | 7 | 158 | 162 | 161 | 160 | 163 | 160 | 163 |
| | 8 | 165 | 166 | 165 | 168 | 165 | 166 | 160 |
| | Animal | | | | | | | |
| Group | # | Day 15 | Day 16 | Day 17 | Day 18 | Day 19 | Day 20 | Day 21 |
| 1 | 1 | 192 | 191 | 185 | 192 | 197 | 196 | 196 |
| (No CIA + | 2 | 183 | 186 | 185 | 189 | 185 | 186 | 190 |
| Vehicle) | 3 | 206 | 205 | 209 | 205 | 214 | 216 | 217 |
| | 4 | 191 | 189 | 190 | 191 | 189 | 191 | 192 |
| | 5 | 201 | 209 | 215 | 214 | 210 | 215 | 219 |
| | 6 | 186 | 189 | 193 | 189 | 194 | 197 | 198 |
| | 7 | 190 | 190 | 190 | 184 | 190 | 192 | 195 |
| | 8 | 185 | 183 | 187 | 191 | 191 | 192 | 189 |
| 2 | 1 | 194 | 189 | 190 | 186 | 181 | 175 | 174 |
| (CIA + Vehicle) | 2 | 185 | 190 | 190 | 186 | 184 | 185 | 184 |
| | 3 | 183 | 181 | 181 | 175 | 172 | 170 | 168 |
| | 4 | 199 | 200 | 197 | 195 | 193 | 189 | 191 |
| | 5 | 168 | 167 | 165 | 168 | 165 | 162 | 159 |
| | 6 | 172 | 180 | 174 | 173 | 170 | 173 | 171 |
| | 7 | 176 | 175 | 175 | 175 | 174 | 169 | 166 |
| | 8 | 174 | 174 | 170 | 166 | 166 | 167 | 169 |
| 3 | 1 | 166 | 157 | 160 | 157 | 162 | 163 | 164 |
| (CIA + | 2 | 157 | 153 | 155 | 152 | 147 | 156 | 156 |
| Dexamethasone) | 3 | 178 | 187 | 188 | 189 | 192 | 184 | 187 |
| | 4 | 170 | 168 | 165 | 165 | 170 | 173 | 169 |
| | 5 | 164 | 159 | 167 | 166 | 168 | 167 | 161 |
| | 6 | 150 | 147 | 150 | 152 | 152 | 154 | 152 |
| | 7 | 176 | 174 | 170 | 179 | 182 | 180 | 177 |
| | 8 | 159 | 163 | 168 | 169 | 163 | 160 | 166 |
| 4 | 1 | 172 | 170 | 168 | 164 | 161 | 163 | 162 |
| (CIA + GSK620) | 2 | 168 | 168 | 168 | 166 | 165 | 163 | 163 |
| | 3 | 184 | 186 | 183 | 182 | 174 | 168 | 170 |
| | 4 | 173 | 171 | 171 | 168 | 170 | 164 | 165 |
| | 5 | 194 | 194 | 192 | 184 | 183 | 186 | 182 |
| | 6 | 174 | 171 | 166 | 161 | 155 | 161 | 160 |
| | 7 | 179 | 173 | 172 | 169 | 165 | 170 | 165 |
| | 8 | 180 | 172 | 169 | 170 | 170 | 169 | 165 |
| 5 | 1 | 175 | 176 | 177 | 177 | 174 | 181 | 179 |
| (CIA + | 2 | 182 | 182 | 177 | 174 | 170 | 173 | 170 |
| Compound A 1 | 3 | 154 | 157 | 155 | 156 | 152 | 154 | 153 |
| mg/kg) | 4 | 174 | 170 | 174 | 173 | 171 | 170 | 166 |
| | 5 | 158 | 156 | 157 | 155 | 154 | 154 | 153 |
| | 6 | 171 | 173 | 173 | 175 | 176 | 175 | 174 |
| | 7 | 168 | 164 | 164 | 164 | 163 | 162 | 159 |
| | 8 | 165 | 161 | 160 | 162 | 164 | 161 | 163 |
| 6 | 1 | 161 | 158 | 154 | 155 | 156 | 155 | 156 |
| (CIA + | 2 | 174 | 169 | 170 | 169 | 169 | 165 | 166 |
| Compound A 3 | 3 | 170 | 170 | 168 | 167 | 168 | 164 | 165 |
| mg/kg) | 4 | 174 | 170 | 169 | 171 | 168 | 162 | 163 |
| | 5 | 159 | 159 | 158 | 157 | 156 | 161 | 162 |
| | 6 | 166 | 170 | 167 | 168 | 165 | 170 | 166 |
| | 7 | 166 | 165 | 162 | 166 | 166 | 163 | 166 |
| | 8 | 158 | 157 | 156 | 155 | 152 | 153 | 155 |
| 7 | 1 | 162 | 163 | 164 | 161 | 163 | 164 | 162 |
| (CIA + | 2 | 170 | 165 | 162 | 160 | 156 | 158 | 155 |
| Compound A 10 | 3 | 169 | 171 | 166 | 168 | 170 | 167 | 167 |
| mg/kg) | 4 | 154 | 153 | 155 | 155 | 155 | 157 | 160 |
| | 5 | 160 | 163 | 168 | 167 | 165 | 165 | 164 |
| | 6 | 157 | 157 | 160 | 162 | 161 | 163 | 165 |
| | 7 | 160 | 154 | 154 | 153 | 153 | 153 | 151 |
| | 8 | 167 | 164 | 162 | 167 | 165 | 163 | 163 |

# Each animal actually had a different number, and the animal numbers are shown for simplicity as 1-8 for each group.

TABLE 18B
Animal body weight percent change (%)

| | Animal | | | | | | | | |
|---|---|---|---|---|---|---|---|---|---|
| Group | # | Day 0 | Day 1 | Day 2 | Day 3 | Day 4 | Day 5 | Day 6 | Day 7 |
| 1 | 1 | 0.00 | 0.00 | 1.21 | 3.03 | 4.85 | 4.85 | 7.88 | 10.91 |
| (No CIA + | 2 | 0.00 | 3.21 | 3.85 | 6.41 | 8.97 | 10.90 | 10.90 | 10.26 |
| Vehicle) | 3 | 0.00 | 1.18 | 2.94 | 2.94 | 7.65 | 7.65 | 8.82 | 11.18 |
| | 4 | 0.00 | −1.79 | 0.00 | 0.60 | 1.79 | 6.55 | 6.55 | 6.55 |
| | 5 | 0.00 | 1.14 | 1.14 | 2.86 | 5.71 | 5.71 | 10.86 | 5.71 |
| | 6 | 0.00 | −0.60 | −1.81 | 0.60 | 2.41 | 6.02 | 6.63 | 7.23 |
| | 7 | 0.00 | 1.80 | 3.59 | 3.59 | 2.40 | 2.40 | 5.39 | 5.39 |
| | 8 | 0.00 | 1.86 | 3.11 | 0.00 | 2.48 | 4.97 | 5.59 | 7.45 |
| 2 | 1 | 0.00 | 2.42 | 3.64 | 6.67 | 4.24 | 9.09 | 11.52 | 10.91 |
| (CIA + Vehicle) | 2 | 0.00 | 4.91 | 4.29 | 4.29 | 6.13 | 7.36 | 10.43 | 12.27 |
| | 3 | 0.00 | 0.61 | 1.22 | 0.00 | 3.66 | 5.49 | 8.54 | 6.10 |
| | 4 | 0.00 | 2.29 | 1.14 | 5.71 | 4.00 | 8.00 | 6.86 | 8.57 |
| | 5 | 0.00 | 2.63 | 1.97 | 3.95 | 5.92 | 6.58 | 11.84 | 13.16 |
| | 6 | 0.00 | 0.00 | −1.88 | 0.00 | 0.63 | 6.25 | 7.50 | 8.13 |
| | 7 | 0.00 | 1.31 | −0.65 | 1.31 | 3.27 | 1.96 | 7.19 | 5.88 |
| | 8 | 0.00 | 1.29 | 3.87 | 6.45 | 7.10 | 7.74 | 11.61 | 12.26 |
| 3 | 1 | 0.00 | 0.66 | −0.66 | −1.99 | −2.65 | −2.65 | −2.65 | −0.66 |
| (CIA + | 2 | 0.00 | −7.41 | −6.79 | 11.11 | −9.26 | −8.02 | 6.17 | −5.56 |
| Dexamethasone) | 3 | 0.00 | −1.72 | 1.15 | 0.57 | −2.30 | −5.17 | −1.72 | 2.30 |
| | 4 | 0.00 | −2.37 | −1.78 | −2.96 | −5.33 | −2.96 | −4.73 | −4.14 |
| | 5 | 0.00 | 1.20 | −3.01 | −2.41 | −6.02 | −4.82 | −6.63 | −3.61 |
| | 6 | 0.00 | −6.21 | −8.07 | −9.94 | −8.07 | 6.83 | 7.45 | −9.94 |
| | 7 | 0.00 | −2.91 | −4.07 | −4.65 | 4.07 | −3.49 | 4.07 | −5.81 |
| | 8 | 0.00 | 1.25 | −2.50 | 2.50 | 1.88 | −5.63 | −1.88 | 0.63 |
| 4 | 1 | 0.00 | 1.91 | 3.18 | 3.82 | 1.27 | 6.37 | 7.64 | 8.28 |
| (CIA + GSK620) | 2 | 0.00 | 2.98 | −0.60 | −0.60 | 2.38 | 5.36 | 1.79 | 4.17 |
| | 3 | 0.00 | 3.64 | 1.82 | 1.82 | 4.24 | 4.24 | 4.24 | 3.03 |
| | 4 | 0.00 | 1.85 | 5.56 | 6.79 | 6.79 | 9.26 | 5.56 | 5.56 |
| | 5 | 0.00 | 2.94 | 1.76 | 1.18 | 0.59 | 5.88 | 4.12 | 5.88 |
| | 6 | 0.00 | 3.73 | 1.86 | 3.11 | 2.48 | 6.21 | 6.21 | 6.83 |
| | 7 | 0.00 | 2.89 | 3.47 | 4.62 | 3.47 | 4.62 | 7.51 | 5.78 |
| | 8 | 0.00 | 3.61 | 2.41 | 4.22 | 3.01 | 6.02 | 6.63 | 6.02 |
| 5 | 1 | 0.00 | 2.45 | 1.84 | 1.23 | 3.07 | 4.29 | 4.29 | 4.91 |
| (CIA + | 2 | 0.00 | 2.42 | 2.42 | 0.61 | 0.61 | 7.88 | 9.09 | 10.30 |
| Compound A 1 | 3 | 0.00 | −3.66 | −3.05 | −3.05 | −3.05 | 0.61 | −4.27 | −4.27 |
| mg/kg) | 4 | 0.00 | 4.32 | 3.09 | 4.32 | 2.47 | 6.17 | 8.64 | 5.56 |
| | 5 | 0.00 | 1.97 | −0.66 | 2.63 | 3.29 | 2.63 | 5.92 | 6.58 |
| | 6 | 0.00 | 0.59 | −1.78 | 2.96 | 0.59 | 1.18 | 1.18 | 0.59 |
| | 7 | 0.00 | 0.00 | −1.20 | 0.00 | −0.60 | 2.40 | 2.40 | 4.19 |
| | 8 | 0.00 | 4.29 | −1.23 | 4.91 | 3.68 | 4.91 | 7.36 | 3.68 |
| 6 | 1 | 0.00 | −2.98 | −3.57 | 4.76 | −0.60 | −1.19 | −2.98 | −6.55 |
| (CIA + | 2 | 0.00 | 0.58 | 0.00 | 1.75 | 1.17 | 3.51 | 1.75 | 0.58 |
| Compound A 3 | 3 | 0.00 | 0.00 | 1.14 | −2.86 | 0.00 | 1.71 | 0.57 | −1.14 |
| mg/kg) | 4 | 0.00 | −3.59 | −2.99 | −2.99 | −4.79 | −4.19 | −4.19 | −2.99 |
| | 5 | 0.00 | −0.60 | −0.60 | −1.80 | −1.20 | −2.40 | 1.80 | −2.40 |
| | 6 | 0.00 | 1.84 | 1.23 | −0.61 | −2.45 | 3.07 | 2.45 | 0.61 |
| | 7 | 0.00 | 1.91 | 1.27 | 1.91 | 5.10 | 6.37 | 7.01 | 5.73 |
| | 8 | 0.00 | 1.28 | 1.28 | 1.28 | 0.00 | −1.28 | 0.64 | −1.28 |
| 7 | 1 | 0.00 | −4.62 | −1.73 | −2.31 | −2.31 | −3.47 | −2.89 | −5.20 |
| | 2 | 0.00 | 2.44 | 1.83 | 1.83 | −0.61 | 4.88 | 1.83 | 2.44 |
| (CIA + | 3 | 0.00 | 2.40 | 1.80 | 2.40 | 1.20 | 2.99 | 2.99 | 3.59 |
| Compound A 10 | 4 | 0.00 | 2.52 | 0.00 | −0.63 | 0.63 | 1.26 | 3.77 | 0.63 |
| mg/kg) | 5 | 0.00 | 2.50 | 2.50 | 6.25 | 5.00 | 6.25 | 6.25 | 5.00 |
| | 6 | 0.00 | −1.28 | 1.28 | 1.28 | 0.64 | 2.56 | 0.64 | −1.92 |
| | 7 | 0.00 | 2.48 | 0.00 | −2.48 | 0.00 | 1.24 | 2.48 | −1.24 |
| | 8 | 0.00 | 2.53 | 2.53 | 1.27 | 2.53 | 5.06 | 2.53 | 3.80 |

| | Animal | | | | | | | |
|---|---|---|---|---|---|---|---|---|
| Group | # | Day 8 | Day 9 | Day 10 | Day 11 | Day 12 | Day 13 | Day 14 |
| 1 | 1 | 7.88 | 9.09 | 11.52 | 13.33 | 16.36 | 16.36 | 15.76 |
| (No CIA + | 2 | 12.18 | 16.03 | 15.38 | 13.46 | 19.87 | 18.59 | 18.59 |
| Vehicle) | 3 | 12.94 | 11.76 | 12.94 | 17.06 | 17.65 | 20.00 | 20.00 |
| | 4 | 7.74 | 10.71 | 11.31 | 11.90 | 8.93 | 12.50 | 13.69 |
| | 5 | 9.71 | 12.00 | 10.86 | 11.43 | 15.43 | 16.57 | 17.14 |
| | 6 | 3.01 | 8.43 | 9.64 | 10.24 | 10.24 | 10.24 | 10.24 |
| | 7 | 5.39 | 7.78 | 9.58 | 10.18 | 10.78 | 10.78 | 10.78 |
| | 8 | 6.83 | 9.32 | 9.94 | 11.80 | 11.80 | 12.42 | 13.04 |
| 2 | 1 | 9.09 | 15.15 | 13.94 | 15.15 | 16.36 | 21.21 | 19.39 |
| (CIA + Vehicle) | 2 | 11.66 | 12.88 | 12.27 | 15.95 | 16.56 | 18.40 | 16.56 |
| | 3 | 6.10 | 10.98 | 11.59 | 9.76 | 14.02 | 17.07 | 16.46 |
| | 4 | 10.29 | 12.57 | 11.43 | 10.29 | 15.43 | 16.00 | 16.00 |
| | 5 | 13.16 | 13.16 | 14.47 | 14.47 | 16.45 | 18.42 | 14.47 |
| | 6 | 6.25 | 7.50 | 8.13 | 10.00 | 8.13 | 8.75 | 5.00 |
| | 7 | 6.54 | 10.46 | 13.07 | 14.38 | 13.07 | 15.69 | 17.65 |
| | 8 | 11.61 | 10.32 | 12.90 | 16.13 | 16.77 | 12.26 | 13.55 |
| 3 | 1 | −2.65 | 1.32 | 1.99 | 2.65 | 1.32 | 0.00 | 3.97 |
| (CIA + | 2 | −11.73 | −10.49 | −6.17 | −7.41 | −6.17 | −7.41 | −9.26 |
| Dexamethasone) | 3 | 0.00 | 1.15 | 1.15 | 5.17 | 6.32 | 6.90 | 4.02 |
| | 4 | −5.33 | 1.78 | −2.96 | −1.18 | −2.37 | 4.14 | −1.78 |
| | 5 | −3.01 | −2.41 | −4.22 | −5.42 | −2.41 | 0.60 | −1.81 |
| | 6 | −5.59 | −1.86 | −6.83 | −6.83 | −8.70 | 8.07 | −6.83 |
| | 7 | −2.91 | 0.00 | 1.74 | 0.58 | −1.16 | 2.33 | 1.74 |
| | 8 | 0.00 | −0.63 | −3.13 | 2.50 | 3.75 | 2.50 | 0.00 |
| 4 | 1 | 7.01 | 8.92 | 5.10 | 10.83 | 12.74 | 12.10 | 8.28 |
| (CIA + GSK620) | 2 | 4.76 | 5.36 | 5.36 | 8.33 | 10.12 | 8.33 | 4.76 |
| | 3 | 7.88 | 9.70 | 9.70 | 9.70 | 11.52 | 10.30 | 9.09 |
| | 4 | 9.26 | 9.88 | 11.11 | 9.88 | 10.49 | 10.49 | 9.26 |
| | 5 | 8.24 | 7.65 | 11.18 | 11.18 | 11.76 | 14.12 | 12.35 |
| | 6 | 6.21 | 8.07 | 10.56 | 9.94 | 12.42 | 8.70 | 9.32 |
| | 7 | 5.78 | 7.51 | 7.51 | 7.51 | 7.51 | 6.94 | 6.36 |
| | 8 | 6.02 | 9.64 | 8.43 | 10.24 | 10.24 | 12.65 | 9.64 |
| 5 | 1 | 1.84 | 3.68 | 4.29 | 4.91 | 6.75 | 1.84 | 5.52 |
| (CIA + | 2 | 4.24 | 9.09 | 7.88 | 9.09 | 10.30 | 9.09 | 7.88 |
| Compound A 1 | 3 | −4.27 | −4.88 | −5.49 | −4.27 | −3.66 | −3.66 | −3.05 |
| mg/kg) | 4 | 4.94 | 8.02 | 9.88 | 9.88 | 8.64 | 10.49 | 10.49 |
| | 5 | 5.92 | 6.58 | 9.21 | 6.58 | 6.58 | 5.26 | 1.97 |
| | 6 | 1.78 | 2.37 | 0.59 | 0.59 | 2.96 | 2.96 | 3.55 |
| | 7 | 2.40 | 1.80 | 5.39 | 4.79 | 5.99 | 2.99 | 1.80 |
| | 8 | 1.23 | 3.68 | 1.84 | 2.45 | 1.23 | 1.23 | 3.07 |
| 6 | 1 | −4.76 | −3.57 | −3.57 | −4.76 | −5.95 | −3.57 | −2.98 |
| (CIA + | 2 | 1.75 | 0.00 | 0.58 | 0.58 | 2.92 | 2.34 | 3.51 |
| Compound A 3 | 3 | −1.14 | 1.71 | 1.14 | 1.71 | 2.86 | −5.14 | −2.86 |
| mg/kg) | 4 | 1.20 | 1.20 | 1.80 | 2.99 | 0.00 | 3.59 | 4.79 |
| | 5 | −2.40 | 4.19 | 0.60 | −4.19 | −5.39 | −5.99 | −6.59 |
| | 6 | 1.23 | 0.00 | 2.45 | 3.68 | 3.68 | 3.07 | 2.45 |
| | 7 | 6.37 | 8.28 | 6.37 | 9.55 | 9.55 | 8.92 | 8.28 |
| | 8 | 0.64 | 2.56 | 3.85 | 5.77 | 5.77 | 3.85 | 1.28 |
| 7 | 1 | −6.36 | −5.20 | −4.62 | −5.20 | −5.78 | −5.78 | −5.20 |
| (CIA + | 2 | 0.00 | 0.61 | 0.61 | 5.49 | 5.49 | 3.66 | 3.05 |
| Compound A 10 | 3 | −1.20 | 0.60 | 2.99 | 1.80 | 1.80 | 2.99 | 1.80 |
| mg/kg) | 4 | 0.63 | 0.63 | 1.26 | 1.89 | 0.63 | −1.26 | −3.14 |
| | 5 | 2.50 | 3.13 | 4.38 | 4.38 | 5.00 | 1.25 | 0.63 |
| | 6 | −0.64 | −2.56 | 1.92 | −2.56 | 1.92 | 0.00 | 0.64 |
| | 7 | −1.86 | 0.62 | 0.00 | −0.62 | 1.24 | −0.62 | 1.24 |
| | 8 | 4.43 | 5.06 | 4.43 | 6.33 | 4.43 | 5.06 | 1.27 |
| | Animal | | | | | | | |
| Group | # | Day 15 | Day 16 | Day 17 | Day 18 | Day 19 | Day 20 | Day 21 |
| 1 | 1 | 16.36 | 15.76 | 12.12 | 16.36 | 19.39 | 18.79 | 18.79 |
| (No CIA + | 2 | 17.31 | 19.23 | 18.59 | 21.15 | 18.59 | 19.23 | 21.79 |
| Vehicle) | 3 | 21.18 | 20.59 | 22.94 | 20.59 | 25.88 | 27.06 | 27.65 |
| | 4 | 13.69 | 12.50 | 13.10 | 13.69 | 12.50 | 13.69 | 14.29 |
| | 5 | 14.86 | 19.43 | 22.86 | 22.29 | 20.00 | 22.86 | 25.14 |
| | 6 | 12.05 | 13.86 | 16.27 | 13.86 | 16.87 | 18.67 | 19.28 |
| | 7 | 13.77 | 13.77 | 13.77 | 10.18 | 13.77 | 14.97 | 16.77 |
| | 8 | 14.91 | 13.66 | 16.15 | 18.63 | 18.63 | 19.25 | 17.39 |
| 2 | 1 | 17.58 | 14.55 | 15.15 | 12.73 | 9.70 | 6.06 | 5.45 |
| (CIA + Vehicle) | 2 | 13.50 | 16.56 | 16.56 | 14.11 | 12.88 | 13.50 | 12.88 |
| | 3 | 11.59 | 10.37 | 10.37 | 6.71 | 4.88 | 3.66 | 2.44 |
| | 4 | 13.71 | 14.29 | 12.57 | 11.43 | 10.29 | 8.00 | 9.14 |
| | 5 | 10.53 | 9.87 | 8.55 | 10.53 | 8.55 | 6.58 | 4.61 |
| | 6 | 7.50 | 12.50 | 8.75 | 8.13 | 6.25 | 8.13 | 6.88 |
| | 7 | 15.03 | 14.38 | 14.38 | 14.38 | 13.73 | 10.46 | 8.50 |
| | 8 | 12.26 | 12.26 | 9.68 | 7.10 | 7.10 | 7.74 | 9.03 |
| 3 | 1 | 9.93 | 3.97 | 5.96 | 3.97 | 7.28 | 7.95 | 8.61 |
| (CIA + | 2 | −3.09 | −5.56 | −4.32 | −6.17 | −9.26 | −3.70 | −3.70 |
| Dexamethasone) | 3 | 2.30 | 7.47 | 8.05 | 8.62 | 10.34 | 5.75 | 7.47 |
| | 4 | 0.59 | −0.59 | −2.37 | −2.37 | 0.59 | 2.37 | 0.00 |
| | 5 | 1.20 | −4.22 | 0.60 | 0.00 | 1.20 | 0.60 | −3.01 |
| | 6 | −6.83 | −8.70 | −6.83 | −5.59 | −5.59 | 4.35 | −5.59 |
| | 7 | 2.33 | 1.16 | −1.16 | 4.07 | 5.81 | 4.65 | 2.91 |
| | 8 | −0.63 | 1.88 | 5.00 | 5.63 | 1.88 | 0.00 | 3.75 |
| 4 | 1 | 9.55 | 8.28 | 7.01 | 4.46 | 2.55 | 3.82 | 3.18 |
| (CIA + GSK620) | 2 | 0.00 | 0.00 | 0.00 | −1.19 | −1.79 | −2.98 | −2.98 |
| | 3 | 11.52 | 12.73 | 10.91 | 10.30 | 5.45 | 1.82 | 3.03 |
| | 4 | 6.79 | 5.56 | 5.56 | 3.70 | 4.94 | 1.23 | 1.85 |
| | 5 | 14.12 | 14.12 | 12.94 | 8.24 | 7.65 | 9.41 | 7.06 |
| | 6 | 8.07 | 6.21 | 3.11 | 0.00 | −3.73 | 0.00 | −0.62 |
| | 7 | 3.47 | 0.00 | −0.58 | −2.31 | −4.62 | −1.73 | −4.62 |
| | 8 | 8.43 | 3.61 | 1.81 | 2.41 | 2.41 | 1.81 | −0.60 |
| 5 | 1 | 7.36 | 7.98 | 8.59 | 8.59 | 6.75 | 11.04 | 9.82 |
| (CIA + | 2 | 10.30 | 10.30 | 7.27 | 5.45 | 3.03 | 4.85 | 3.03 |
| Compound A 1 | 3 | −6.10 | −4.27 | −5.49 | −4.88 | −7.32 | −6.10 | −6.71 |
| mg/kg) | 4 | 7.41 | 4.94 | 7.41 | 6.79 | 5.56 | 4.94 | 2.47 |
| | 5 | 3.95 | 2.63 | 3.29 | 1.97 | 1.32 | 1.32 | 0.66 |
| | 6 | 1.18 | 2.37 | 2.37 | 3.55 | 4.14 | 3.55 | 2.96 |
| | 7 | 0.60 | −1.80 | −1.80 | −1.80 | −2.40 | −2.99 | −4.79 |
| | 8 | 1.23 | −1.23 | −1.84 | −0.61 | 0.61 | −1.23 | 0.00 |
| 6 | 1 | −4.17 | −5.95 | −8.33 | −7.74 | −7.14 | −7.74 | −7.14 |
| (CIA + | 2 | 1.75 | −1.17 | −0.58 | −1.17 | −1.17 | −3.51 | −2.92 |
| Compound A 3 | 3 | −2.86 | −2.86 | −4.00 | −4.57 | −4.00 | −6.29 | −5.71 |
| mg/kg) | 4 | 4.19 | 1.80 | 1.20 | 2.40 | 0.60 | −2.99 | −2.40 |
| | 5 | −4.79 | −4.79 | −5.39 | −5.99 | −6.59 | −3.59 | −2.99 |
| | 6 | 1.84 | 4.29 | 2.45 | 3.07 | 1.23 | 4.29 | 1.84 |
| | 7 | 5.73 | 5.10 | 3.18 | 5.73 | 5.73 | 3.82 | 5.73 |
| | 8 | 1.28 | 0.64 | 0.00 | −0.64 | −2.56 | −1.92 | −0.64 |
| 7 | 1 | −6.36 | −5.78 | −5.20 | −6.94 | −5.78 | −5.20 | −6.36 |
| (CIA + | 2 | 3.66 | 0.61 | −1.22 | −2.44 | −4.88 | −3.66 | −5.49 |
| Compound A 10 | 3 | 1.20 | 2.40 | −0.60 | 0.60 | 1.80 | 0.00 | 0.00 |
| mg/kg) | 4 | −3.14 | −3.77 | −2.52 | −2.52 | −2.52 | −1.26 | 0.63 |
| | 5 | 0.00 | 1.88 | 5.00 | 4.38 | 3.13 | 3.13 | 2.50 |
| | 6 | 0.64 | 0.64 | 2.56 | 3.85 | 3.21 | 4.49 | 5.77 |
| | 7 | −0.62 | −4.35 | −4.35 | −4.97 | −4.97 | −4.97 | −6.21 |
| | 8 | 5.70 | 3.80 | 2.53 | 5.70 | 4.43 | 3.16 | 3.16 |

# Each animal actually had a different number, and the animal numbers are shown for simplicity as 1-8 for each group.

TABLE 18C
Average animal body weight

| Group | Day 0 | Day 1 | Day 2 | Day 3 | Day 4 | Day 5 | Day 6 | Day 7 |
|---|---|---|---|---|---|---|---|---|
| 1 | 166 | 167 | 169 | 170 | 174 | 176 | 179 | 179 |
| (No CIA + | | | | | | | | |
| Vehicle) | | | | | | | | |
| 2 | 161 | 164 | 164 | 167 | 168 | 172 | 176 | 176 |
| (CIA + Vehicle) | | | | | | | | |
| 3 | 164 | 160 | 159 | 157 | 156 | 156 | 157 | 159 |
| (CIA + | | | | | | | | |
| Dexamethasone) | | | | | | | | |
| 4 | 165 | 170 | 169 | 170 | 170 | 175 | 174 | 175 |
| (CIA + GSK620) | | | | | | | | |
| 5 | 163 | 166 | 163 | 165 | 165 | 169 | 170 | 170 |
| (CIA + | | | | | | | | |
| Compound A 1 | | | | | | | | |
| mg/kg) | | | | | | | | |
| 6 | 166 | 165 | 165 | 163 | 165 | 167 | 166 | 164 |
| (CIA + | | | | | | | | |
| Compound A 3 | | | | | | | | |
| mg/kg) | | | | | | | | |
| 7 | 162 | 164 | 163 | 163 | 163 | 166 | 166 | 164 |
| (CIA + | | | | | | | | |
| Compound A 10 | | | | | | | | |
| mg/kg) | | | | | | | | |

| Group | Day 8 | Day 9 | Day 10 | Day 11 | Day 12 | Day 13 | Day 14 |
|---|---|---|---|---|---|---|---|
| 1 | 180 | 184 | 185 | 187 | 189 | 190 | 191 |
| (No CIA + | | | | | | | |
| Vehicle) | | | | | | | |
| 2 | 176 | 180 | 181 | 182 | 184 | 187 | 185 |
| (CIA + Vehicle) | | | | | | | |
| 3 | 158 | 161 | 161 | 162 | 163 | 163 | 162 |
| (CIA + | | | | | | | |
| Dexamethasone) | | | | | | | |
| 4 | 177 | 179 | 180 | 181 | 183 | 183 | 180 |
| (CIA + GSK620) | | | | | | | |
| 5 | 167 | 169 | 170 | 170 | 171 | 169 | 170 |
| (CIA + | | | | | | | |
| Compound A 1 | | | | | | | |
| mg/kg) | | | | | | | |
| 6 | 166 | 167 | 168 | 169 | 167 | 167 | 167 |
| (CIA + | | | | | | | |
| Compound A 3 | | | | | | | |
| mg/kg) | | | | | | | |
| 7 | 162 | 163 | 164 | 165 | 164 | 163 | 162 |
| (CIA + | | | | | | | |
| Compound A 10 | | | | | | | |
| mg/kg) | | | | | | | |
| Group | Day 15 | Day 16 | Day 17 | Day 18 | Day 19 | Day 20 | Day 21 |
| 1 | 192 | 193 | 194 | 194 | 196 | 198 | 200 |
| (No CIA + | | | | | | | |
| Vehicle) | | | | | | | |
| 2 | 181 | 182 | 180 | 178 | 176 | 174 | 173 |
| (CIA + Vehicle) | | | | | | | |
| 3 | 165 | 164 | 165 | 166 | 167 | 167 | 167 |
| (CIA + | | | | | | | |
| Dexamethasone) | | | | | | | |
| 4 | 178 | 176 | 174 | 171 | 168 | 168 | 167 |
| (CIA + GSK620) | | | | | | | |
| 5 | 168 | 167 | 167 | 167 | 166 | 166 | 165 |
| (CIA + Compound | | | | | | | |
| A 1 mg/kg) | | | | | | | |
| 6 | 166 | 165 | 163 | 164 | 163 | 162 | 162 |
| (CIA + Compound | | | | | | | |
| A 3 mg/kg) | | | | | | | |
| 7 | 162 | 161 | 161 | 162 | 161 | 161 | 161 |
| (CIA + Compound | | | | | | | |
| A 10 mg/kg) | | | | | | | |

TABLE 18D
Average animal body weight percent change

| Group | Day 0 | Day 1 | Day 2 | Day 3 | Day 4 | Day 5 | Day 6 | Day 7 |
|---|---|---|---|---|---|---|---|---|
| 1 | 0.00 | 0.85 | 1.75 | 2.50 | 4.53 | 6.13 | 7.83 | 8.08 |
| (No CIA + | | | | | | | | |
| Vehicle) | | | | | | | | |
| 2 | 0.00 | 1.78 | 1.70 | 3.55 | 4.37 | 6.56 | 9.44 | 9.66 |
| (CIA + Vehicle) | | | | | | | | |
| 3 | 0.00 | −2.80 | −3.22 | −4.37 | −4.95 | −4.95 | −4.41 | −3.35 |
| (CIA + | | | | | | | | |
| Dexamethasone) | | | | | | | | |
| 4 | 0.00 | 2.94 | 2.43 | 3.12 | 3.03 | 6.00 | 5.46 | 5.69 |
| (CIA + GSK620) | | | | | | | | |
| 5 | 0.00 | 1.55 | −0.07 | 0.96 | 1.26 | 3.76 | 4.33 | 3.94 |
| (CIA + | | | | | | | | |
| Compound A 1 | | | | | | | | |
| mg/kg) | | | | | | | | |
| 6 | 0.00 | −0.19 | −0.57 | −1.45 | −0.35 | 0.70 | 0.43 | −0.93 |
| (CIA + | | | | | | | | |
| Compound A 3 | | | | | | | | |
| mg/kg) | | | | | | | | |
| 7 | 0.00 | 1.12 | 0.71 | 0.63 | 0.72 | 2.60 | 2.04 | 0.89 |
| (CIA + | | | | | | | | |
| Compound A 10 | | | | | | | | |
| mg/kg) | | | | | | | | |

| Group | Day 8 | Day 9 | Day 10 | Day 11 | Day 12 | Day 13 | Day 14 |
|---|---|---|---|---|---|---|---|
| 1 | 8.21 | 10.64 | 11.40 | 12.43 | 13.88 | 14.68 | 14.91 |
| (No CIA + | | | | | | | |
| Vehicle) | | | | | | | |
| 2 | 9.34 | 11.63 | 12.22 | 13.27 | 14.60 | 15.98 | 14.89 |
| (CIA + Vehicle) | | | | | | | |
| 3 | −3.90 | −1.84 | −2.30 | −1.24 | −1.18 | −0.91 | −1.24 |
| (CIA + | | | | | | | |
| Dexamethasone) | | | | | | | |
| 4 | 6.89 | 8.34 | 8.62 | 9.70 | 10.85 | 10.45 | 8.63 |
| (CIA + GSK620) | | | | | | | |
| 5 | 2.26 | 3.79 | 4.20 | 4.25 | 4.85 | 3.78 | 3.90 |
| (CIA + | | | | | | | |
| Compound A 1 | | | | | | | |
| mg/kg) | | | | | | | |
| 6 | 0.36 | 0.75 | 1.65 | 1.92 | 0.97 | 0.88 | 0.99 |
| (CIA + | | | | | | | |
| Compound A 3 | | | | | | | |
| mg/kg) | | | | | | | |
| 7 | −0.31 | 0.36 | 0.89 | 1.44 | 1.36 | 0.66 | 0.03 |
| (CIA + | | | | | | | |
| Compound A 10 | | | | | | | |
| mg/kg) | | | | | | | |
| Group | Day 15 | Day 16 | Day 17 | Day 18 | Day 19 | Day 20 | Day 21 |
| 1 | 15.52 | 16.10 | 16.97 | 17.09 | 18.20 | 19.32 | 20.14 |
| (No CIA + | | | | | | | |
| Vehicle) | | | | | | | |
| 2 | 12.71 | 13.10 | 12.00 | 10.64 | 9.17 | 8.01 | 7.37 |
| (CIA + Vehicle) | | | | | | | |
| 3 | 0.43 | −0.57 | 0.62 | 1.02 | 1.53 | 1.66 | 1.30 |
| (CIA + | | | | | | | |
| Dexamethasone) | | | | | | | |
| 4 | 7.74 | 6.31 | 5.09 | 3.20 | 1.61 | 1.67 | 0.79 |
| (CIA + GSK620) | | | | | | | |
| 5 | 3.24 | 2.62 | 2.48 | 2.38 | 1.46 | 1.92 | 0.93 |
| (CIA + | | | | | | | |
| Compound A 1 | | | | | | | |
| mg/kg) | | | | | | | |
| 6 | 0.37 | −0.37 | −1.43 | −1.11 | −1.74 | −2.24 | −1.78 |
| (CIA + | | | | | | | |
| Compound A 3 | | | | | | | |
| mg/kg) | | | | | | | |
| 7 | 0.13 | −0.57 | −0.47 | −0.29 | −0.70 | −0.54 | −0.75 |
| (CIA + | | | | | | | |
| Compound A 10 | | | | | | | |
| mg/kg) | | | | | | | |

TABLE 19A
Animal paw volume (mL)

| | Animal | Day 0 | | Day 6 | | Day 12 | | Day 13 | | Day 14 | |
|---|---|---|---|---|---|---|---|---|---|---|---|
| Group | # | L | R | L | R | L | R | L | R | L | R |
| 1 | 1 | 1.32 | 1.34 | 1.36 | 1.37 | 1.38 | 1.38 | 1.38 | 1.41 | 1.37 | 1.41 |
| | 2 | 1.31 | 1.32 | 1.32 | 1.32 | 1.37 | 1.39 | 1.37 | 1.40 | 1.38 | 1.42 |
| (No CIA + | 3 | 1.31 | 1.31 | 1.33 | 1.34 | 1.37 | 1.38 | 1.38 | 1.40 | 1.39 | 1.42 |
| Vehicle) | 4 | 1.28 | 1.29 | 1.32 | 1.31 | 1.41 | 1.42 | 1.43 | 1.45 | 1.43 | 1.46 |
| | 5 | 1.36 | 1.34 | 1.36 | 1.34 | 1.46 | 1.49 | 1.48 | 1.49 | 1.51 | 1.49 |
| | 6 | 1.28 | 1.29 | 1.30 | 1.30 | 1.36 | 1.35 | 1.37 | 1.36 | 1.38 | 1.38 |
| | 7 | 1.33 | 1.32 | 1.33 | 1.32 | 1.37 | 1.39 | 1.39 | 1.40 | 1.40 | 1.42 |
| | 8 | 1.31 | 1.32 | 1.34 | 1.33 | 1.38 | 1.37 | 1.38 | 1.39 | 1.40 | 1.40 |
| 2 | 1 | 1.35 | 1.35 | 1.38 | 1.38 | 1.86 | 1.83 | 2.07 | 1.94 | 2.55 | 2.42 |
| (CIA + Vehicle) | 2 | 1.33 | 1.33 | 1.36 | 1.36 | 1.47 | 1.45 | 1.49 | 1.48 | 1.69 | 1.99 |
| | 3 | 1.30 | 1.31 | 1.32 | 1.33 | 1.45 | 1.46 | 1.49 | 1.49 | 1.49 | 1.49 |
| | 4 | 1.35 | 1.35 | 1.38 | 1.38 | 2.13 | 1.90 | 2.42 | 2.03 | 2.66 | 2.60 |
| | 5 | 1.33 | 1.32 | 1.36 | 1.34 | 1.47 | 1.48 | 1.41 | 1.49 | 1.99 | 1.69 |
| | 6 | 1.31 | 1.30 | 1.34 | 1.33 | 1.45 | 1.44 | 1.45 | 1.46 | 1.49 | 1.50 |
| | 7 | 1.29 | 1.30 | 1.30 | 1.32 | 1.48 | 1.48 | 1.82 | 1.53 | 2.21 | 1.83 |
| | 8 | 1.30 | 1.30 | 1.31 | 1.30 | 1.46 | 1.41 | 1.74 | 1.47 | 2.14 | 1.49 |
| 3 | 1 | 1.35 | 1.34 | 1.31 | 1.30 | 1.21 | 1.25 | 1.23 | 1.26 | 1.24 | 1.26 |
| (CIA + | 2 | 1.31 | 1.30 | 1.29 | 1.28 | 1.21 | 1.26 | 1.24 | 1.27 | 1.24 | 1.26 |
| Dexamethasone) | 3 | 1.39 | 1.38 | 1.34 | 1.34 | 1.37 | 1.37 | 1.40 | 1.38 | 1.39 | 1.38 |
| | 4 | 1.33 | 1.35 | 1.29 | 1.30 | 1.28 | 1.25 | 1.29 | 1.25 | 1.27 | 1.23 |
| | 5 | 1.30 | 1.29 | 1.26 | 1.27 | 1.28 | 1.27 | 1.30 | 1.28 | 1.28 | 1.26 |
| | 6 | 1.30 | 1.30 | 1.26 | 1.26 | 1.22 | 1.18 | 1.25 | 1.21 | 1.24 | 1.20 |
| | 7 | 1.34 | 1.36 | 1.28 | 1.28 | 1.30 | 1.26 | 1.31 | 1.31 | 1.29 | 1.29 |
| | 8 | 1.36 | 1.37 | 1.29 | 1.30 | 1.29 | 1.29 | 1.29 | 1.29 | 1.25 | 1.26 |
| 4 | 1 | 1.27 | 1.27 | 1.27 | 1.27 | 1.33 | 1.31 | 1.34 | 1.40 | 1.55 | 1.74 |
| (CIA + GSK620) | 2 | 1.32 | 1.31 | 1.34 | 1.35 | 1.33 | 1.35 | 1.34 | 1.35 | 1.78 | 1.49 |
| | 3 | 1.27 | 1.28 | 1.37 | 1.38 | 1.41 | 1.42 | 1.43 | 1.42 | 1.44 | 1.44 |
| | 4 | 1.37 | 1.37 | 1.36 | 1.37 | 1.37 | 1.39 | 1.36 | 1.57 | 1.36 | 2.11 |
| | 5 | 1.37 | 1.37 | 1.38 | 1.39 | 1.42 | 1.39 | 1.43 | 1.42 | 1.43 | 1.43 |
| | 6 | 1.34 | 1.33 | 1.34 | 1.33 | 1.36 | 1.38 | 1.37 | 1.37 | 1.40 | 1.38 |
| | 7 | 1.32 | 1.33 | 1.32 | 1.33 | 1.35 | 1.38 | 1.38 | 1.41 | 1.73 | 1.99 |
| | 8 | 1.35 | 1.34 | 1.35 | 1.34 | 1.38 | 1.37 | 1.40 | 1.39 | 1.73 | 1.83 |
| 5 | 1 | 1.35 | 1.35 | 1.36 | 1.36 | 1.39 | 1.38 | 1.41 | 1.40 | 1.43 | 1.42 |
| (CIA + | 2 | 1.31 | 1.31 | 1.33 | 1.33 | 1.36 | 1.35 | 1.39 | 1.38 | 1.42 | 1.40 |
| Compound A 1 | 3 | 1.29 | 1.30 | 1.30 | 1.30 | 1.33 | 1.34 | 1.35 | 1.36 | 1.73 | 1.49 |
| mg/kg) | 4 | 1.36 | 1.38 | 1.40 | 1.41 | 1.43 | 1.44 | 1.43 | 1.44 | 1.49 | 1.52 |
| | 5 | 1.33 | 1.33 | 1.35 | 1.35 | 1.44 | 1.57 | 1.57 | 1.58 | 1.58 | 1.60 |
| | 6 | 1.34 | 1.36 | 1.37 | 1.37 | 1.40 | 1.43 | 1.43 | 1.44 | 1.47 | 1.48 |
| | 7 | 1.33 | 1.32 | 1.35 | 1.35 | 1.37 | 1.36 | 1.43 | 1.37 | 1.64 | 1.45 |
| | 8 | 1.33 | 1.33 | 1.35 | 1.36 | 1.37 | 1.49 | 1.42 | 1.52 | 1.50 | 1.56 |
| 6 | 1 | 1.32 | 1.33 | 1.32 | 1.33 | 1.33 | 1.36 | 1.35 | 1.37 | 1.45 | 1.38 |
| (CIA + | 2 | 1.29 | 1.29 | 1.35 | 1.34 | 1.36 | 1.36 | 1.40 | 1.36 | 1.41 | 1.41 |
| Compound A 3 | 3 | 1.32 | 1.33 | 1.34 | 1.36 | 1.35 | 1.36 | 1.35 | 1.37 | 1.45 | 1.37 |
| mg/kg) | 4 | 1.37 | 1.35 | 1.37 | 1.35 | 1.39 | 1.38 | 1.40 | 1.43 | 1.41 | 1.43 |
| | 5 | 1.31 | 1.30 | 1.32 | 1.33 | 1.35 | 1.35 | 1.42 | 1.36 | 1.51 | 1.47 |
| | 6 | 1.34 | 1.34 | 1.36 | 1.35 | 1.37 | 1.36 | 1.39 | 1.39 | 1.41 | 1.44 |
| | 7 | 1.31 | 1.31 | 1.33 | 1.33 | 1.36 | 1.34 | 1.40 | 1.38 | 1.40 | 1.40 |
| | 8 | 1.31 | 1.30 | 1.33 | 1.33 | 1.52 | 1.40 | 1.55 | 1.54 | 1.54 | 1.70 |
| 7 | 1 | 1.31 | 1.31 | 1.32 | 1.31 | 1.37 | 1.34 | 1.37 | 1.34 | 1.38 | 1.35 |
| (CIA + | 2 | 1.29 | 1.29 | 1.29 | 1.29 | 1.33 | 1.33 | 1.36 | 1.34 | 1.42 | 1.37 |
| Compound A 10 | 3 | 1.33 | 1.32 | 1.37 | 1.37 | 1.38 | 1.40 | 1.43 | 1.41 | 1.51 | 1.42 |
| mg/kg) | 4 | 1.35 | 1.34 | 1.40 | 1.40 | 1.41 | 1.39 | 1.41 | 1.60 | 1.42 | 1.73 |
| | 5 | 1.35 | 1.34 | 1.37 | 1.37 | 1.38 | 1.51 | 1.66 | 1.56 | 1.68 | 1.62 |
| | 6 | 1.33 | 1.34 | 1.37 | 1.37 | 1.37 | 1.37 | 1.39 | 1.37 | 1.47 | 1.38 |
| | 7 | 1.29 | 1.29 | 1.29 | 1.29 | 1.31 | 1.32 | 1.32 | 1.35 | 1.32 | 1.37 |
| | 8 | 1.31 | 1.30 | 1.32 | 1.30 | 1.36 | 1.35 | 1.38 | 1.36 | 1.38 | 1.35 |

| | Animal | Day 15 | | Day 18 | | Day 19 | | Day 21 | |
|---|---|---|---|---|---|---|---|---|---|
| Group | # | L | R | L | R | L | R | L | R |
| 1 | 1 | 1.38 | 1.42 | 1.39 | 1.40 | 1.38 | 1.40 | 1.38 | 1.38 |
| (No CIA + | 2 | 1.38 | 1.42 | 1.39 | 1.40 | 1.38 | 1.40 | 1.38 | 1.38 |
| Vehicle) | 3 | 1.42 | 1.42 | 1.42 | 1.42 | 1.41 | 1.42 | 1.42 | 1.42 |
| | 4 | 1.43 | 1.45 | 1.42 | 1.44 | 1.41 | 1.41 | 1.42 | 1.43 |
| | 5 | 1.53 | 1.51 | 1.52 | 1.51 | 1.51 | 1.50 | 1.54 | 1.52 |
| | 6 | 1.39 | 1.38 | 1.39 | 1.38 | 1.39 | 1.40 | 1.40 | 1.40 |
| | 7 | 1.40 | 1.43 | 1.41 | 1.42 | 1.41 | 1.41 | 1.40 | 1.40 |
| | 8 | 1.40 | 1.42 | 1.42 | 1.41 | 1.41 | 1.40 | 1.41 | 1.40 |
| 2 | 1 | 2.70 | 2.54 | 2.71 | 2.72 | 2.74 | 2.73 | 2.72 | 2.71 |
| (CIA + Vehicle) | 2 | 2.14 | 2.22 | 2.77 | 3.00 | 2.92 | 3.02 | 3.03 | 3.03 |
| | 3 | 1.69 | 2.05 | 2.87 | 2.88 | 3.03 | 3.25 | 3.04 | 3.22 |
| | 4 | 3.09 | 2.89 | 3.21 | 3.18 | 3.26 | 3.23 | 3.26 | 3.22 |
| | 5 | 2.29 | 2.16 | 2.94 | 2.75 | 2.94 | 2.74 | 2.94 | 2.75 |
| | 6 | 1.92 | 1.69 | 2.37 | 2.53 | 2.54 | 2.52 | 2.64 | 2.54 |
| | 7 | 2.67 | 2.05 | 2.69 | 2.68 | 2.66 | 2.63 | 2.66 | 2.64 |
| | 8 | 2.62 | 1.65 | 2.62 | 2.30 | 2.62 | 2.63 | 2.60 | 2.64 |
| 3 | 1 | 1.23 | 1.25 | 1.28 | 1.28 | 1.28 | 1.27 | 1.26 | 1.26 |
| (CIA + | 2 | 1.21 | 1.24 | 1.22 | 1.24 | 1.22 | 1.22 | 1.22 | 1.22 |
| Dexamethasone) | 3 | 1.36 | 1.36 | 1.45 | 1.44 | 1.44 | 1.44 | 1.43 | 1.43 |
| | 4 | 1.28 | 1.27 | 1.31 | 1.31 | 1.30 | 1.31 | 1.31 | 1.31 |
| | 5 | 1.26 | 1.27 | 1.29 | 1.29 | 1.28 | 1.27 | 1.30 | 1.30 |
| | 6 | 1.16 | 1.14 | 1.18 | 1.17 | 1.19 | 1.18 | 1.18 | 1.18 |
| | 7 | 1.33 | 1.32 | 1.34 | 1.34 | 1.33 | 1.32 | 1.31 | 1.32 |
| | 8 | 1.27 | 1.26 | 1.30 | 1.29 | 1.30 | 1.30 | 1.30 | 1.30 |
| 4 | 1 | 2.04 | 2.05 | 2.72 | 2.62 | 2.71 | 2.59 | 2.71 | 2.63 |
| (CIA + GSK620) | 2 | 2.03 | 2.08 | 2.54 | 2.45 | 2.54 | 2.46 | 2.51 | 2.44 |
| | 3 | 1.39 | 1.38 | 2.39 | 2.41 | 2.32 | 2.33 | 2.34 | 2.21 |
| | 4 | 1.49 | 2.42 | 2.87 | 3.15 | 3.06 | 3.15 | 3.04 | 3.08 |
| | 5 | 1.52 | 1.47 | 2.29 | 1.46 | 2.61 | 1.46 | 2.62 | 1.61 |
| | 6 | 1.60 | 1.56 | 2.05 | 2.04 | 2.19 | 2.20 | 2.21 | 2.18 |
| | 7 | 2.14 | 2.13 | 2.66 | 2.38 | 2.63 | 2.36 | 2.54 | 2.37 |
| | 8 | 1.75 | 1.79 | 2.34 | 2.34 | 2.32 | 2.33 | 2.32 | 2.33 |
| 5 | 1 | 1.43 | 1.43 | 1.42 | 1.43 | 1.40 | 1.41 | 1.42 | 1.42 |
| (CIA + | 2 | 1.45 | 1.42 | 2.03 | 2.08 | 2.03 | 2.20 | 2.05 | 2.27 |
| Compound A 1 | 3 | 1.84 | 1.81 | 2.42 | 2.46 | 2.42 | 2.43 | 2.45 | 2.44 |
| mg/kg) | 4 | 1.77 | 1.56 | 2.06 | 1.84 | 2.03 | 1.84 | 2.08 | 2.08 |
| | 5 | 1.64 | 1.65 | 2.21 | 2.04 | 2.24 | 2.05 | 2.27 | 2.26 |
| | 6 | 1.48 | 1.50 | 1.67 | 1.61 | 1.64 | 1.60 | 1.64 | 1.61 |
| | 7 | 1.66 | 1.72 | 2.02 | 2.04 | 2.02 | 2.04 | 2.08 | 2.05 |
| | 8 | 1.92 | 1.59 | 2.03 | 1.82 | 2.04 | 1.80 | 2.15 | 1.99 |
| 6 | 1 | 1.49 | 1.39 | 1.71 | 2.14 | 1.89 | 2.15 | 1.92 | 2.19 |
| (CIA + | 2 | 1.59 | 1.41 | 1.99 | 1.58 | 1.99 | 1.62 | 2.19 | 1.64 |
| Compound A 3 | 3 | 1.48 | 1.46 | 1.40 | 1.91 | 1.62 | 1.90 | 1.64 | 1.94 |
| mg/kg) | 4 | 1.49 | 1.42 | 1.51 | 1.76 | 1.52 | 1.75 | 1.52 | 1.75 |
| | 5 | 1.54 | 1.75 | 1.55 | 1.95 | 1.69 | 1.98 | 1.80 | 2.08 |
| | 6 | 1.68 | 1.57 | 1.66 | 1.65 | 1.69 | 1.64 | 1.65 | 1.69 |
| | 7 | 1.49 | 1.40 | 1.64 | 1.58 | 1.59 | 1.58 | 1.57 | 1.58 |
| | 8 | 1.56 | 1.70 | 1.66 | 1.69 | 1.68 | 1.69 | 1.58 | 1.69 |
| 7 | 1 | 1.62 | 1.51 | 1.62 | 1.60 | 1.62 | 1.53 | 1.62 | 1.53 |
| (CIA + | 2 | 1.42 | 1.41 | 2.09 | 2.02 | 2.04 | 1.93 | 2.14 | 2.17 |
| Compound A 10 | 3 | 1.53 | 1.40 | 1.51 | 2.03 | 1.50 | 1.92 | 1.52 | 1.92 |
| mg/kg) | 4 | 1.77 | 1.77 | 1.72 | 2.01 | 1.76 | 2.09 | 2.06 | 2.23 |
| | 5 | 1.73 | 1.80 | 1.42 | 1.64 | 1.80 | 1.60 | 1.80 | 1.64 |
| | 6 | 1.48 | 1.36 | 1.40 | 1.36 | 1.44 | 1.35 | 1.44 | 1.35 |
| | 7 | 1.41 | 1.41 | 1.77 | 1.82 | 1.76 | 1.89 | 1.82 | 2.00 |
| | 8 | 1.39 | 1.39 | 1.39 | 1.63 | 1.43 | 1.70 | 1.43 | 1.71 |

# Each animal actually had a different number, and the animal numbers are shown for simplicity as 1-8 for each group.

TABLE 19B
Animal paw volume percent change (%)

| | Animal | Day 0 | | Day 6 | | Day 12 | | Day 13 | | Day 14 | |
|---|---|---|---|---|---|---|---|---|---|---|---|
| Group | # | L | R | L | R | L | R | L | R | L | R |
| 1 | 1 | 0.00 | 0.00 | 3.04 | 2.25 | 4.56 | 3.37 | 4.56 | 5.24 | 3.80 | 5.24 |
| (No CIA + | 2 | 0.00 | 0.00 | 0.77 | 0.38 | 4.60 | 5.32 | 4.60 | 6.46 | 5.75 | 7.60 |
| Vehicle) | 3 | 0.00 | 0.00 | 1.53 | 2.30 | 4.60 | 5.36 | 5.36 | 6.90 | 6.51 | 8.43 |
| | 4 | 0.00 | 0.00 | 3.14 | 1.56 | 10.20 | 10.51 | 11.76 | 12.45 | 12.16 | 13.62 |
| | 5 | 0.00 | 0.00 | 0.00 | 0.37 | 7.75 | 11.24 | 9.23 | 11.24 | 11.44 | 11.24 |
| | 6 | 0.00 | 0.00 | 1.57 | 1.17 | 6.67 | 4.67 | 7.06 | 5.45 | 8.24 | 7.00 |
| | 7 | 0.00 | 0.00 | 0.00 | 0.00 | 3.02 | 5.32 | 4.53 | 6.08 | 5.28 | 7.98 |
| | 8 | 0.00 | 0.00 | 1.91 | 1.14 | 4.96 | 4.18 | 4.96 | 5.32 | 6.49 | 6.08 |
| 2 | 1 | 0.00 | 0.00 | 2.23 | 2.60 | 37.92 | 35.69 | 53.90 | 43.87 | 89.22 | 79.55 |
| (CIA + Vehicle) | 2 | 0.00 | 0.00 | 1.88 | 2.64 | 10.15 | 9.43 | 11.65 | 11.32 | 26.69 | 50.19 |
| | 3 | 0.00 | 0.00 | 1.54 | 1.15 | 11.58 | 11.07 | 14.67 | 13.36 | 14.67 | 13.36 |
| | 4 | 0.00 | 0.00 | 2.23 | 2.23 | 57.99 | 41.26 | 79.55 | 50.56 | 97.40 | 92.94 |
| | 5 | 0.00 | 0.00 | 1.88 | 1.52 | 10.15 | 11.74 | 6.02 | 12.50 | 49.25 | 27.65 |
| | 6 | 0.00 | 0.00 | 1.91 | 2.31 | 10.31 | 10.38 | 10.31 | 11.92 | 13.36 | 15.00 |
| | 7 | 0.00 | 0.00 | 1.17 | 1.15 | 15.18 | 13.85 | 41.25 | 17.31 | 71.60 | 40.38 |
| | 8 | 0.00 | 0.00 | 0.77 | 0.00 | 11.92 | 8.08 | 33.46 | 12.69 | 64.23 | 14.23 |
| 3 | 1 | 0.00 | 0.00 | −2.97 | −3.00 | −10.41 | −6.74 | −8.92 | −5.99 | −8.18 | −5.62 |
| (CIA + | 2 | 0.00 | 0.00 | −1.53 | 1.16 | −7.66 | −3.09 | −5.36 | −2.32 | −5.36 | −2.70 |
| Dexamethasone) | 3 | 0.00 | 0.00 | −3.61 | −2.91 | −1.44 | 0.73 | 0.72 | 0.36 | 0.00 | 0.00 |
| | 4 | 0.00 | 0.00 | −3.02 | −3.72 | −3.40 | −7.43 | −3.02 | −7.43 | −4.53 | −8.92 |
| | 5 | 0.00 | 0.00 | −3.09 | −1.56 | −1.54 | −1.56 | 0.00 | −0.78 | −1.54 | −2.33 |
| | 6 | 0.00 | 0.00 | −3.08 | −2.70 | −6.54 | −9.27 | 4.23 | −6.95 | −5.00 | −7.72 |
| | 7 | 0.00 | 0.00 | −4.12 | −5.90 | −3.00 | −7.01 | −2.25 | −3.69 | −3.75 | −5.17 |
| | 8 | 0.00 | 0.00 | 5.51 | −5.13 | −5.51 | −5.86 | −5.51 | 5.86 | −8.46 | −8.06 |
| 4 | 1 | 0.00 | 0.00 | −0.39 | −0.39 | 4.33 | 2.76 | 5.12 | 9.84 | 21.65 | 37.01 |
| (CIA + GSK620) | 2 | 0.00 | 0.00 | 1.52 | 3.07 | 0.76 | 3.07 | 1.52 | 3.07 | 34.98 | 14.18 |
| | 3 | 0.00 | 0.00 | 7.91 | 7.84 | 11.07 | 10.98 | 12.65 | 10.98 | 13.44 | 12.55 |
| | 4 | 0.00 | 0.00 | −0.73 | 0.00 | 0.00 | 1.47 | −0.73 | 14.65 | −0.37 | 54.58 |
| | 5 | 0.00 | 0.00 | 0.36 | 1.47 | 3.28 | 1.47 | 4.01 | 3.66 | 4.01 | 4.40 |
| | 6 | 0.00 | 0.00 | 0.00 | 0.00 | 1.50 | 3.77 | 2.25 | 3.02 | 4.87 | 3.77 |
| | 7 | 0.00 | 0.00 | 0.00 | 0.00 | 2.28 | 3.77 | 4.56 | 6.04 | 31.18 | 49.81 |
| | 8 | 0.00 | 0.00 | −0.37 | 0.00 | 1.85 | 2.25 | 3.33 | 3.75 | 27.78 | 36.70 |
| 5 | 1 | 0.00 | 0.00 | 0.74 | 0.74 | 2.97 | 2.23 | 4.83 | 3.72 | 5.95 | 5.20 |
| (CIA + | 2 | 0.00 | 0.00 | 1.53 | 1.53 | 4.21 | 3.45 | 6.13 | 5.36 | 8.43 | 6.90 |
| Compound A 1 | 3 | 0.00 | 0.00 | 0.39 | 0.00 | 2.71 | 3.09 | 4.26 | 4.63 | 34.11 | 15.06 |
| mg/kg) | 4 | 0.00 | 0.00 | 2.95 | 2.18 | 5.17 | 4.36 | 5.17 | 4.36 | 9.59 | 10.18 |
| | 5 | 0.00 | 0.00 | 1.51 | 1.51 | 8.30 | 18.11 | 18.11 | 18.87 | 18.87 | 20.38 |
| | 6 | 0.00 | 0.00 | 2.25 | 0.74 | 4.49 | 5.17 | 6.74 | 5.90 | 9.74 | 8.86 |
| | 7 | 0.00 | 0.00 | 1.51 | 2.28 | 3.02 | 3.04 | 7.55 | 3.80 | 23.40 | 9.89 |
| | 8 | 0.00 | 0.00 | 1.89 | 2.26 | 3.02 | 12.08 | 6.79 | 14.34 | 12.83 | 17.36 |
| 6 | 1 | 0.00 | 0.00 | 0.00 | 0.00 | 0.76 | 2.26 | 2.28 | 3.02 | 9.89 | 3.77 |
| (CIA + | 2 | 0.00 | 0.00 | 4.67 | 3.89 | 5.45 | 5.84 | 8.56 | 5.84 | 9.34 | 9.34 |
| Compound A 3 | 3 | 0.00 | 0.00 | 1.90 | 2.26 | 2.28 | 2.26 | 2.28 | 3.02 | 9.89 | 3.02 |
| mg/kg) | 4 | 0.00 | 0.00 | 0.00 | 0.00 | 1.47 | 2.23 | 2.20 | 5.95 | 2.93 | 5.95 |
| | 5 | 0.00 | 0.00 | 0.77 | 2.32 | 3.07 | 3.86 | 8.43 | 4.63 | 15.71 | 13.51 |
| | 6 | 0.00 | 0.00 | 1.50 | 0.75 | 2.25 | 1.50 | 3.75 | 3.75 | 5.24 | 7.49 |
| | 7 | 0.00 | 0.00 | 1.15 | 1.53 | 3.44 | 2.30 | 6.49 | 5.36 | 6.49 | 6.90 |
| | 8 | 0.00 | 0.00 | 1.53 | 2.32 | 16.09 | 7.72 | 18.39 | 18.53 | 17.62 | 31.27 |
| 7 | 1 | 0.00 | 0.00 | 1.15 | 0.00 | 4.60 | 2.30 | 4.60 | 2.30 | 5.75 | 3.07 |
| (CIA + | 2 | 0.00 | 0.00 | 0.00 | 0.00 | 3.11 | 3.11 | 5.45 | 3.89 | 10.12 | 6.23 |
| Compound A 10 | 3 | 0.00 | 0.00 | 3.02 | 3.79 | 3.77 | 5.68 | 7.55 | 6.44 | 13.58 | 7.20 |
| mg/kg) | 4 | 0.00 | 0.00 | 3.72 | 4.10 | 4.46 | 3.36 | 4.46 | 19.03 | 5.20 | 28.73 |
| | 5 | 0.00 | 0.00 | 1.49 | 2.24 | 2.23 | 12.31 | 23.42 | 16.04 | 24.54 | 20.52 |
| | 6 | 0.00 | 0.00 | 3.02 | 2.62 | 3.02 | 2.25 | 4.53 | 2.25 | 10.57 | 3.00 |
| | 7 | 0.00 | 0.00 | 0.00 | 0.00 | 1.56 | 2.33 | 2.33 | 4.67 | 2.33 | 6.61 |
| | 8 | 0.00 | 0.00 | 0.77 | 0.00 | 3.83 | 3.86 | 5.36 | 4.63 | 5.36 | 3.86 |

| | | | | |
|---|---|---|---|---|
| Animal | Day 15 | Day 18 | Day 19 | Day 21 |

| Group | # | L | R | L | R | L | R | L | R |
|---|---|---|---|---|---|---|---|---|---|
| 1 | 1 | 4.56 | 5.99 | 5.32 | 4.49 | 4.56 | 4.49 | 4.56 | 3.00 |
| (No CIA + | 2 | 5.36 | 7.60 | 6.13 | 6.08 | 5.36 | 6.08 | 5.36 | 4.56 |
| Vehicle) | 3 | 8.43 | 8.43 | 8.43 | 8.43 | 7.66 | 8.43 | 8.43 | 8.43 |
| | 4 | 11.76 | 12.45 | 10.98 | 11.67 | 10.59 | 9.73 | 10.98 | 10.89 |
| | 5 | 12.55 | 12.73 | 11.81 | 12.73 | 11.07 | 12.36 | 13.28 | 13.48 |
| | 6 | 8.63 | 7.00 | 8.63 | 7.00 | 9.02 | 8.56 | 9.41 | 8.56 |
| | 7 | 5.28 | 8.37 | 6.04 | 7.60 | 6.04 | 6.84 | 5.28 | 6.08 |
| | 8 | 6.49 | 7.60 | 8.02 | 6.84 | 7.25 | 6.08 | 7.25 | 6.08 |
| 2 | 1 | 100.37 | 88.48 | 101.12 | 101.86 | 103.35 | 102.60 | 102.23 | 101.12 |
| (CIA + Vehicle) | 2 | 60.90 | 67.17 | 107.89 | 126.04 | 119.55 | 127.92 | 127.44 | 128.30 |
| | 3 | 30.12 | 56.11 | 121.24 | 119.85 | 133.59 | 147.71 | 134.36 | 145.42 |
| | 4 | 129.37 | 114.50 | 138.29 | 136.06 | 142.01 | 139.78 | 142.01 | 139.03 |
| | 5 | 72.18 | 63.26 | 120.68 | 107.95 | 120.68 | 107.20 | 120.68 | 107.95 |
| | 6 | 46.18 | 29.62 | 80.53 | 94.23 | 93.51 | 93.46 | 101.15 | 95.00 |
| | 7 | 107.39 | 57.31 | 108.95 | 105.77 | 106.61 | 101.92 | 106.61 | 102.69 |
| | 8 | 101.15 | 26.92 | 101.15 | 76.54 | 101.15 | 101.92 | 99.62 | 102.69 |
| 3 | 1 | −8.92 | −6.74 | −5.20 | 4.49 | 5.20 | −5.24 | −6.32 | −5.99 |
| (CIA + | 2 | −7.28 | −4.25 | −6.51 | −4.63 | −6.90 | −6.18 | −6.90 | −6.18 |
| Dexamethasone) | 3 | −2.17 | 1.45 | 4.33 | 4.36 | 3.61 | 4.36 | 2.89 | 3.64 |
| | 4 | −3.77 | −5.95 | −1.51 | −2.97 | −2.26 | −2.97 | −1.51 | −2.97 |
| | 5 | −3.09 | −1.56 | 0.77 | 0.00 | −1.54 | 1.56 | 0.00 | 0.78 |
| | 6 | 10.77 | 12.36 | −9.62 | −10.04 | −8.85 | 9.27 | 9.62 | −9.27 |
| | 7 | 0.75 | −2.95 | 0.00 | −1.48 | 0.75 | −2.58 | −2.25 | −2.95 |
| | 8 | −6.99 | −8.06 | −4.78 | −5.86 | −4.78 | −5.13 | −4.78 | −5.13 |
| 4 | 1 | 60.24 | 61.02 | 113.78 | 105.91 | 112.99 | 103.54 | 112.99 | 106.69 |
| (CIA + GSK620) | 2 | 53.99 | 59.00 | 92.78 | 87.36 | 92.78 | 88.12 | 90.49 | 86.59 |
| | 3 | 9.49 | 7.84 | 88.54 | 88.63 | 83.00 | 82.75 | 84.58 | 72.94 |
| | 4 | 8.79 | 76.92 | 109.89 | 130.40 | 123.81 | 130.40 | 122.34 | 125.27 |
| | 5 | 10.58 | 7.33 | 67.15 | 6.59 | 90.15 | 6.59 | 90.88 | 17.95 |
| | 6 | 19.48 | 17.36 | 53.18 | 53.96 | 63.67 | 65.66 | 65.17 | 64.15 |
| | 7 | 62.36 | 60.38 | 101.90 | 79.25 | 100.00 | 77.74 | 92.78 | 78.49 |
| | 8 | 29.63 | 33.71 | 72.96 | 75.28 | 71.48 | 74.16 | 71.48 | 74.16 |
| 5 | 1 | 5.95 | 5.95 | 5.20 | 5.95 | 3.72 | 4.46 | 5.20 | 5.20 |
| (CIA + | 2 | 10.73 | 8.43 | 55.17 | 59.00 | 55.17 | 68.20 | 56.70 | 73.56 |
| Compound A 1 | 3 | 42.25 | 39.77 | 87.60 | 89.96 | 87.60 | 87.26 | 89.53 | 88.03 |
| mg/kg) | 4 | 30.26 | 13.09 | 51.66 | 33.45 | 49.82 | 33.45 | 53.14 | 50.91 |
| | 5 | 23.40 | 24.15 | 66.42 | 53.58 | 69.06 | 54.34 | 70.94 | 70.19 |
| | 6 | 10.86 | 10.33 | 24.72 | 18.45 | 22.47 | 17.71 | 22.47 | 18.45 |
| | 7 | 24.91 | 30.42 | 52.08 | 55.13 | 52.08 | 54.75 | 56.60 | 55.51 |
| | 8 | 44.53 | 20.00 | 52.83 | 36.98 | 53.58 | 35.85 | 61.89 | 49.81 |
| 6 | 1 | 12.93 | 4.53 | 29.66 | 61.13 | 43.35 | 61.89 | 46.01 | 64.91 |
| (CIA + | 2 | 23.35 | 9.73 | 54.47 | 22.57 | 54.47 | 25.68 | 70.04 | 27.24 |
| Compound A 3 | 3 | 12.17 | 9.81 | 6.08 | 44.15 | 22.81 | 43.02 | 24.33 | 46.04 |
| mg/kg) | 4 | 8.79 | 5.20 | 10.26 | 30.48 | 10.99 | 29.74 | 10.99 | 29.74 |
| | 5 | 18.01 | 34.75 | 18.39 | 50.58 | 29.12 | 52.90 | 37.55 | 60.23 |
| | 6 | 25.47 | 17.23 | 23.97 | 23.22 | 26.22 | 22.47 | 23.22 | 26.22 |
| | 7 | 13.36 | 6.90 | 25.19 | 20.69 | 20.99 | 20.69 | 19.47 | 20.69 |
| | 8 | 19.54 | 30.89 | 26.82 | 30.12 | 28.35 | 30.12 | 20.69 | 30.12 |
| 7 | 1 | 23.75 | 15.33 | 23.75 | 22.22 | 23.75 | 16.86 | 23.75 | 16.86 |
| (CIA + | 2 | 10.12 | 9.34 | 62.26 | 56.81 | 58.37 | 50.19 | 66.15 | 68.48 |
| Compound A 10 | 3 | 15.09 | 5.68 | 13.58 | 53.41 | 12.83 | 45.08 | 14.34 | 45.08 |
| mg/kg) | 4 | 31.23 | 31.72 | 27.51 | 49.63 | 30.48 | 55.60 | 52.79 | 66.04 |
| | 5 | 28.62 | 33.96 | 5.20 | 22.01 | 33.46 | 19.03 | 33.83 | 22.01 |
| | 6 | 11.32 | 1.50 | 5.66 | 1.50 | 8.30 | 0.75 | 8.30 | 0.75 |
| | 7 | 9.34 | 9.34 | 37.35 | 41.25 | 36.58 | 46.69 | 41.25 | 55.25 |
| | 8 | 6.13 | 6.95 | 6.13 | 25.48 | 9.20 | 30.89 | 9.20 | 31.66 |

# Each animal actually had a different number, and the animal numbers are shown for simplicity as 1-8 for each group.

TABLE 19C
Average animal paw volume (mL)

| | Day 0 | | Day 6 | | Day 12 | | Day 13 | | Day 14 | |
|---|---|---|---|---|---|---|---|---|---|---|
| Group | L | R | L | R | L | R | L | R | L | R |
| 1 | 1.31 | 1.31 | 1.33 | 1.33 | 1.38 | 1.39 | 1.39 | 1.41 | 1.41 | 1.42 |
| (No CIA + | | | | | | | | | | |
| Vehicle) | | | | | | | | | | |
| 2 | 1.32 | 1.32 | 1.34 | 1.34 | 1.59 | 1.55 | 1.73 | 1.61 | 2.02 | 1.87 |
| (CIA + Vehicle) | | | | | | | | | | |
| 3 | 1.33 | 1.33 | 1.29 | 1.29 | 1.27 | 1.26 | 1.28 | 1.28 | 1.27 | 1.26 |
| (CIA + | | | | | | | | | | |
| Dexamethasone) | | | | | | | | | | |
| 4 | 1.32 | 1.32 | 1.34 | 1.34 | 1.36 | 1.37 | 1.38 | 1.41 | 1.55 | 1.67 |
| (CIA + GSK620) | | | | | | | | | | |
| 5 | 1.33 | 1.33 | 1.35 | 1.35 | 1.38 | 1.42 | 1.42 | 1.43 | 1.53 | 1.49 |
| (CIA + | | | | | | | | | | |
| Compound A 1 | | | | | | | | | | |
| mg/kg) | | | | | | | | | | |
| 6 | 1.32 | 1.31 | 1.34 | 1.34 | 1.37 | 1.36 | 1.40 | 1.40 | 1.44 | 1.45 |
| (CIA + | | | | | | | | | | |
| Compound A 3 | | | | | | | | | | |
| mg/kg) | | | | | | | | | | |
| 7 | 1.32 | 1.31 | 1.34 | 1.33 | 1.36 | 1.37 | 1.41 | 1.41 | 1.44 | 1.44 |
| (CIA + | | | | | | | | | | |
| Compound A 10 | | | | | | | | | | |
| mg/kg) | | | | | | | | | | |

| | Day 15 | | Day 18 | | Day 19 | | Day 21 | |
|---|---|---|---|---|---|---|---|---|
| Group | L | R | L | R | L | R | L | R |
| 1 | 1.41 | 1.43 | 1.42 | 1.42 | 1.41 | 1.41 | 1.41 | 1.41 |
| (No CIA + | | | | | | | | |
| Vehicle) | | | | | | | | |
| 2 | 2.39 | 2.15 | 2.77 | 2.75 | 2.83 | 2.84 | 2.86 | 2.84 |
| (CIA + Vehicle) | | | | | | | | |
| 3 | 1.26 | 1.26 | 1.29 | 1.29 | 1.29 | 1.28 | 1.28 | 1.29 |
| (CIA + | | | | | | | | |
| Dexamethasone) | | | | | | | | |
| 4 | 1.74 | 1.86 | 2.48 | 2.35 | 2.54 | 2.36 | 2.53 | 2.35 |
| (CIA + GSK620) | | | | | | | | |
| 5 | 1.64 | 1.58 | 1.98 | 1.91 | 1.97 | 1.92 | 2.01 | 2.01 |
| (CIA + | | | | | | | | |
| Compound A 1 | | | | | | | | |
| mg/kg) | | | | | | | | |
| 6 | 1.54 | 1.51 | 1.64 | 1.78 | 1.70 | 1.78 | 1.73 | 1.82 |
| (CIA + | | | | | | | | |
| Compound A 3 | | | | | | | | |
| mg/kg) | | | | | | | | |
| 7 | 1.54 | 1.50 | 1.61 | 1.76 | 1.66 | 1.75 | 1.72 | 1.81 |
| (CIA + | | | | | | | | |
| Compound A 10 | | | | | | | | |
| mg/kg) | | | | | | | | |

TABLE 19D
Average animal paw volume percent change (%)

| | Day 0 | | Day 6 | | Day 12 | | Day 13 | | Day 14 | |
|---|---|---|---|---|---|---|---|---|---|---|
| Group | L | R | L | R | L | R | L | R | L | R |
| 1 | 0.00 | 0.00 | 1.49 | 1.15 | 5.79 | 6.25 | 6.51 | 7.39 | 7.46 | 8.40 |
| (No CIA + | | | | | | | | | | |
| Vehicle) | | | | | | | | | | |
| 2 | 0.00 | 0.00 | 1.70 | 1.70 | 20.65 | 17.69 | 31.35 | 21.69 | 53.30 | 41.66 |
| (CIA + Vehicle) | | | | | | | | | | |
| 3 | 0.00 | 0.00 | -3.37 | -3.26 | -4.94 | -5.21 | -3.57 | -4.08 | -4.60 | -5.07 |
| (CIA + | | | | | | | | | | |
| Dexamethasone) | | | | | | | | | | |
| 4 | 0.00 | 0.00 | 1.04 | 1.50 | 3.13 | 3.69 | 4.09 | 6.88 | 17.19 | 26.62 |
| (CIA + GSK620) | | | | | | | | | | |
| 5 | 0.00 | 0.00 | 1.60 | 1.41 | 4.24 | 6.44 | 7.45 | 7.62 | 15.36 | 11.73 |
| (CIA + | | | | | | | | | | |
| Compound A 1 | | | | | | | | | | |
| mg/kg) | | | | | | | | | | |
| 6 | 0.00 | 0.00 | 1.44 | 1.63 | 4.35 | 3.50 | 6.55 | 6.26 | 9.64 | 10.16 |
| (CIA + | | | | | | | | | | |
| Compound A 3 | | | | | | | | | | |
| mg/kg) | | | | | | | | | | |
| 7 | 0.00 | 0.00 | 1.64 | 1.59 | 3.32 | 4.40 | 7.21 | 7.41 | 9.68 | 9.90 |
| (CIA + | | | | | | | | | | |
| Compound A 10 | | | | | | | | | | |
| mg/kg) | | | | | | | | | | |

| | Day 15 | | Day 18 | | Day 19 | | Day 21 | |
|---|---|---|---|---|---|---|---|---|
| Group | L | R | L | R | L | R | L | R |
| 1 | 7.88 | 8.77 | 8.17 | 8.11 | 7.69 | 7.82 | 8.07 | 7.64 |
| (No CIA + | | | | | | | | |
| Vehicle) | | | | | | | | |
| 2 | 80.96 | 62.92 | 109.98 | 108.54 | 115.06 | 115.31 | 116.76 | 115.28 |
| (CIA + Vehicle) | | | | | | | | |
| 3 | −5.47 | −5.41 | −3.01 | −3.14 | −3.33 | −3.57 | −3.56 | −3.51 |
| (CIA + | | | | | | | | |
| Dexamethasone) | | | | | | | | |
| 4 | 31.82 | 40.45 | 87.52 | 78.42 | 92.23 | 78.62 | 91.34 | 78.28 |
| (CIA + GSK620) | | | | | | | | |
| 5 | 24.11 | 19.02 | 49.46 | 44.06 | 49.19 | 44.50 | 52.06 | 51.46 |
| (CIA + | | | | | | | | |
| Compound A 1 | | | | | | | | |
| mg/kg) | | | | | | | | |
| 6 | 16.70 | 14.88 | 24.36 | 35.37 | 29.54 | 35.81 | 31.54 | 38.15 |
| (CIA + | | | | | | | | |
| Compound A 3 | | | | | | | | |
| mg/kg) | | | | | | | | |
| 7 | 16.95 | 14.23 | 22.68 | 34.04 | 26.62 | 33.14 | 31.20 | 38.27 |
| (CIA + | | | | | | | | |
| Compound A 10 | | | | | | | | |
| mg/kg) | | | | | | | | |

TABLE 20A
Detailed clinical scores

| | | Day 0 | | | | | Day 6 | | | | | Day 12 | | | | |
|---|---|---|---|---|---|---|---|---|---|---|---|---|---|---|---|---|
| | | | | left | right | | | | left | right | | | | left | right | |
| | | left | right | hind | hind | | left | right | hind | hind | | left | right | hind | hind | |
| Group | ID | forelimb | forelimb | limb | limb | score | forelimb | forelimb | limb | limb | score | forelimb | forelimb | limb | limb | score |
| 1 | 1 | 0 | 0 | 0 | 0 | 0 | 0 | 0 | 0 | 0 | 0 | 0 | 0 | 0 | 0 | 0 |
| (No CIA + | 2 | 0 | 0 | 0 | 0 | 0 | 0 | 0 | 0 | 0 | 0 | 0 | 0 | 0 | 0 | 0 |
| Vehicle) | 3 | 0 | 0 | 0 | 0 | 0 | 0 | 0 | 0 | 0 | 0 | 0 | 0 | 0 | 0 | 0 |
| | 4 | 0 | 0 | 0 | 0 | 0 | 0 | 0 | 0 | 0 | 0 | 0 | 0 | 0 | 0 | 0 |
| | 5 | 0 | 0 | 0 | 0 | 0 | 0 | 0 | 0 | 0 | 0 | 0 | 0 | 0 | 0 | 0 |
| | 6 | 0 | 0 | 0 | 0 | 0 | 0 | 0 | 0 | 0 | 0 | 0 | 0 | 0 | 0 | 0 |
| | 7 | 0 | 0 | 0 | 0 | 0 | 0 | 0 | 0 | 0 | 0 | 0 | 0 | 0 | 0 | 0 |
| | 8 | 0 | 0 | 0 | 0 | 0 | 0 | 0 | 0 | 0 | 0 | 0 | 0 | 0 | 0 | 0 |
| 2 | 1 | 0 | 0 | 0 | 0 | 0 | 0 | 0 | 0 | 0 | 0 | 0 | 0 | 2 | 2 | 4 |
| (CIA + Vehicle) | 2 | 0 | 0 | 0 | 0 | 0 | 0 | 0 | 0 | 0 | 0 | 0 | 0 | 0 | 0 | 0 |
| | 3 | 0 | 0 | 0 | 0 | 0 | 0 | 0 | 0 | 0 | 0 | 0 | 0 | 0 | 0 | 0 |
| | 4 | 0 | 0 | 0 | 0 | 0 | 0 | 0 | 0 | 0 | 0 | 1 | 0 | 3 | 2 | 6 |
| | 5 | 0 | 0 | 0 | 0 | 0 | 0 | 0 | 0 | 0 | 0 | 0 | 0 | 0 | 0 | 0 |
| | 6 | 0 | 0 | 0 | 0 | 0 | 0 | 0 | 0 | 0 | 0 | 0 | 0 | 0 | 0 | 0 |
| | 7 | 0 | 0 | 0 | 0 | 0 | 0 | 0 | 0 | 0 | 0 | 0 | 0 | 0 | 0 | 0 |
| | 8 | 0 | 0 | 0 | 0 | 0 | 0 | 0 | 0 | 0 | 0 | 0 | 0 | 0 | 0 | 0 |
| 3 | 1 | 0 | 0 | 0 | 0 | 0 | 0 | 0 | 0 | 0 | 0 | 0 | 0 | 0 | 0 | 0 |
| (CIA + | 2 | 0 | 0 | 0 | 0 | 0 | 0 | 0 | 0 | 0 | 0 | 0 | 0 | 0 | 0 | 0 |
| Dexamethasone) | 3 | 0 | 0 | 0 | 0 | 0 | 0 | 0 | 0 | 0 | 0 | 0 | 0 | 0 | 0 | 0 |
| | 4 | 0 | 0 | 0 | 0 | 0 | 0 | 0 | 0 | 0 | 0 | 0 | 0 | 0 | 0 | 0 |
| | 5 | 0 | 0 | 0 | 0 | 0 | 0 | 0 | 0 | 0 | 0 | 0 | 0 | 0 | 0 | 0 |
| | 6 | 0 | 0 | 0 | 0 | 0 | 0 | 0 | 0 | 0 | 0 | 0 | 0 | 0 | 0 | 0 |
| | 7 | 0 | 0 | 0 | 0 | 0 | 0 | 0 | 0 | 0 | 0 | 0 | 0 | 0 | 0 | 0 |
| | 8 | 0 | 0 | 0 | 0 | 0 | 0 | 0 | 0 | 0 | 0 | 0 | 0 | 0 | 0 | 0 |
| 4 | 1 | 0 | 0 | 0 | 0 | 0 | 0 | 0 | 0 | 0 | 0 | 0 | 0 | 0 | 0 | 0 |
| (CIA + GSK620) | 2 | 0 | 0 | 0 | 0 | 0 | 0 | 0 | 0 | 0 | 0 | 0 | 0 | 0 | 0 | 0 |
| | 3 | 0 | 0 | 0 | 0 | 0 | 0 | 0 | 0 | 0 | 0 | 0 | 0 | 0 | 0 | 0 |
| | 4 | 0 | 0 | 0 | 0 | 0 | 0 | 0 | 0 | 0 | 0 | 0 | 0 | 0 | 0 | 0 |
| | 5 | 0 | 0 | 0 | 0 | 0 | 0 | 0 | 0 | 0 | 0 | 0 | 0 | 0 | 0 | 0 |
| | 6 | 0 | 0 | 0 | 0 | 0 | 0 | 0 | 0 | 0 | 0 | 0 | 0 | 0 | 0 | 0 |
| | 7 | 0 | 0 | 0 | 0 | 0 | 0 | 0 | 0 | 0 | 0 | 0 | 0 | 0 | 0 | 0 |
| | 8 | 0 | 0 | 0 | 0 | 0 | 0 | 0 | 0 | 0 | 0 | 0 | 0 | 0 | 0 | 0 |
| 5 | 1 | 0 | 0 | 0 | 0 | 0 | 0 | 0 | 0 | 0 | 0 | 0 | 0 | 0 | 0 | 0 |
| (CIA + | 2 | 0 | 0 | 0 | 0 | 0 | 0 | 0 | 0 | 0 | 0 | 0 | 0 | 0 | 0 | 0 |
| Compound A 1 | 3 | 0 | 0 | 0 | 0 | 0 | 0 | 0 | 0 | 0 | 0 | 0 | 0 | 0 | 0 | 0 |
| mg/kg) | 4 | 0 | 0 | 0 | 0 | 0 | 0 | 0 | 0 | 0 | 0 | 0 | 0 | 0 | 0 | 0 |
| | 5 | 0 | 0 | 0 | 0 | 0 | 0 | 0 | 0 | 0 | 0 | 0 | 0 | 0 | 1 | 1 |
| | 6 | 0 | 0 | 0 | 0 | 0 | 0 | 0 | 0 | 0 | 0 | 0 | 0 | 0 | 0 | 0 |
| | 7 | 0 | 0 | 0 | 0 | 0 | 0 | 0 | 0 | 0 | 0 | 0 | 0 | 0 | 0 | 0 |
| | 8 | 0 | 0 | 0 | 0 | 0 | 0 | 0 | 0 | 0 | 0 | 0 | 0 | 0 | 0 | 0 |
| 6 | 1 | 0 | 0 | 0 | 0 | 0 | 0 | 0 | 0 | 0 | 0 | 0 | 0 | 0 | 0 | 0 |
| (CIA + | 2 | 0 | 0 | 0 | 0 | 0 | 0 | 0 | 0 | 0 | 0 | 0 | 0 | 0 | 0 | 0 |
| Compound A 3 | 3 | 0 | 0 | 0 | 0 | 0 | 0 | 0 | 0 | 0 | 0 | 0 | 0 | 0 | 0 | 0 |
| mg/kg) | 4 | 0 | 0 | 0 | 0 | 0 | 0 | 0 | 0 | 0 | 0 | 0 | 0 | 0 | 0 | 0 |
| | 5 | 0 | 0 | 0 | 0 | 0 | 0 | 0 | 0 | 0 | 0 | 0 | 0 | 0 | 0 | 0 |
| | 6 | 0 | 0 | 0 | 0 | 0 | 0 | 0 | 0 | 0 | 0 | 0 | 0 | 0 | 0 | 0 |
| | 7 | 0 | 0 | 0 | 0 | 0 | 0 | 0 | 0 | 0 | 0 | 0 | 0 | 0 | 0 | 0 |
| | 8 | 0 | 0 | 0 | 0 | 0 | 0 | 0 | 0 | 0 | 0 | 0 | 0 | 1 | 0 | 1 |
| 7 | 1 | 0 | 0 | 0 | 0 | 0 | 0 | 0 | 0 | 0 | 0 | 0 | 0 | 0 | 0 | 0 |
| (CIA + | 2 | 0 | 0 | 0 | 0 | 0 | 0 | 0 | 0 | 0 | 0 | 0 | 0 | 0 | 0 | 0 |
| Compound A 10 | 3 | 0 | 0 | 0 | 0 | 0 | 0 | 0 | 0 | 0 | 0 | 0 | 0 | 0 | 0 | 0 |
| mg/kg) | 4 | 0 | 0 | 0 | 0 | 0 | 0 | 0 | 0 | 0 | 0 | 0 | 0 | 0 | 0 | 0 |
| | 5 | 0 | 0 | 0 | 0 | 0 | 0 | 0 | 0 | 0 | 0 | 0 | 1 | 0 | 1 | 2 |
| | 6 | 0 | 0 | 0 | 0 | 0 | 0 | 0 | 0 | 0 | 0 | 0 | 0 | 0 | 0 | 0 |
| | 7 | 0 | 0 | 0 | 0 | 0 | 0 | 0 | 0 | 0 | 0 | 0 | 0 | 0 | 0 | 0 |
| | 8 | 0 | 0 | 0 | 0 | 0 | 0 | 0 | 0 | 0 | 0 | 0 | 0 | 0 | 0 | 0 |
| | | Day 13 | | | | | Day 14 | | | | | Day 15 | | | | |
| | | | | left | right | | | | left | right | | | | left | right | |
| | | left | right | hind | hind | | left | right | hind | hind | | left | right | hind | hind | |
| Group | ID | forelimb | forelimb | limb | limb | score | forelimb | forelimb | limb | limb | score | forelimb | forelimb | limb | limb | score |
| 1 | 1 | 0 | 0 | 0 | 0 | 0 | 0 | 0 | 0 | 0 | 0 | 0 | 0 | 0 | 0 | 0 |
| (No CIA + | 2 | 0 | 0 | 0 | 0 | 0 | 0 | 0 | 0 | 0 | 0 | 0 | 0 | 0 | 0 | 0 |
| Vehicle) | 3 | 0 | 0 | 0 | 0 | 0 | 0 | 0 | 0 | 0 | 0 | 0 | 0 | 0 | 0 | 0 |
| | 4 | 0 | 0 | 0 | 0 | 0 | 0 | 0 | 0 | 0 | 0 | 0 | 0 | 0 | 0 | 0 |
| | 5 | 0 | 0 | 0 | 0 | 0 | 0 | 0 | 0 | 0 | 0 | 0 | 0 | 0 | 0 | 0 |
| | 6 | 0 | 0 | 0 | 0 | 0 | 0 | 0 | 0 | 0 | 0 | 0 | 0 | 0 | 0 | 0 |
| | 7 | 0 | 0 | 0 | 0 | 0 | 0 | 0 | 0 | 0 | 0 | 0 | 0 | 0 | 0 | 0 |
| | 8 | 0 | 0 | 0 | 0 | 0 | 0 | 0 | 0 | 0 | 0 | 0 | 0 | 0 | 0 | 0 |
| 2 | 1 | 0 | 0 | 3 | 2 | 5 | 0 | 0 | 4 | 4 | 8 | 0 | 1 | 4 | 4 | 9 |
| (CIA + Vehicle) | 2 | 0 | 0 | 0 | 0 | 0 | 0 | 0 | 1 | 2 | 3 | 0 | 0 | 3 | 3 | 6 |
| | 3 | 0 | 0 | 0 | 0 | 0 | 0 | 0 | 0 | 0 | 0 | 0 | 0 | 1 | 3 | 4 |
| | 4 | 2 | 1 | 4 | 3 | 10 | 3 | 2 | 4 | 4 | 13 | 4 | 4 | 4 | 4 | 16 |
| | 5 | 0 | 0 | 0 | 0 | 0 | 1 | 0 | 2 | 1 | 4 | 1 | 1 | 3 | 3 | 8 |
| | 6 | 0 | 0 | 0 | 0 | 0 | 0 | 0 | 0 | 0 | 0 | 0 | 0 | 2 | 1 | 3 |
| | 7 | 0 | 0 | 2 | 1 | 3 | 0 | 0 | 3 | 2 | 5 | 0 | 0 | 4 | 3 | 7 |
| | 8 | 0 | 0 | 1 | 0 | 1 | 0 | 0 | 3 | 0 | 3 | 0 | 0 | 4 | 0 | 4 |
| 3 | 1 | 0 | 0 | 0 | 0 | 0 | 0 | 0 | 0 | 0 | 0 | 0 | 0 | 0 | 0 | 0 |
| (CIA+ | 2 | 0 | 0 | 0 | 0 | 0 | 0 | 0 | 0 | 0 | 0 | 0 | 0 | 0 | 0 | 0 |
| Dexamethasone) | 3 | 0 | 0 | 0 | 0 | 0 | 0 | 0 | 0 | 0 | 0 | 0 | 0 | 0 | 0 | 0 |
| | 4 | 0 | 0 | 0 | 0 | 0 | 0 | 0 | 0 | 0 | 0 | 0 | 0 | 0 | 0 | 0 |
| | 5 | 0 | 0 | 0 | 0 | 0 | 0 | 0 | 0 | 0 | 0 | 0 | 0 | 0 | 0 | 0 |
| | 6 | 0 | 0 | 0 | 0 | 0 | 0 | 0 | 0 | 0 | 0 | 0 | 0 | 0 | 0 | 0 |
| | 7 | 0 | 0 | 0 | 0 | 0 | 0 | 0 | 0 | 0 | 0 | 0 | 0 | 0 | 0 | 0 |
| | 8 | 0 | 0 | 0 | 0 | 0 | 0 | 0 | 0 | 0 | 0 | 0 | 0 | 0 | 0 | 0 |
| 4 | 1 | 0 | 0 | 0 | 0 | 0 | 0 | 0 | 1 | 2 | 3 | 0 | 0 | 3 | 3 | 6 |
| (CIA + GSK620) | 2 | 0 | 0 | 0 | 0 | 0 | 0 | 0 | 2 | 0 | 2 | 0 | 0 | 3 | 3 | 6 |
| | 3 | 0 | 0 | 0 | 0 | 0 | 0 | 0 | 0 | 0 | 0 | 0 | 0 | 0 | 0 | 0 |
| | 4 | 0 | 0 | 0 | 1 | 1 | 0 | 0 | 0 | 3 | 3 | 0 | 0 | 0 | 4 | 4 |
| | 5 | 0 | 0 | 0 | 0 | 0 | 0 | 0 | 0 | 0 | 0 | 0 | 0 | 1 | 0 | 1 |
| | 6 | 0 | 0 | 0 | 0 | 0 | 0 | 0 | 0 | 0 | 0 | 0 | 0 | 1 | 1 | 2 |
| | 7 | 0 | 0 | 0 | 0 | 0 | 0 | 0 | 2 | 2 | 4 | 0 | 0 | 3 | 3 | 6 |
| | 8 | 0 | 0 | 0 | 0 | 0 | 0 | 0 | 2 | 2 | 4 | 0 | 0 | 2 | 2 | 4 |
| 5 | 1 | 0 | 0 | 0 | 0 | 0 | 0 | 0 | 0 | 0 | 0 | 0 | 0 | 0 | 0 | 0 |
| (CIA + | 2 | 0 | 0 | 0 | 0 | 0 | 0 | 0 | 0 | 0 | 0 | 0 | 0 | 0 | 0 | 0 |
| Compound A 1 | 3 | 0 | 0 | 0 | 0 | 0 | 0 | 0 | 2 | 0 | 2 | 0 | 0 | 2 | 2 | 4 |
| mg/kg) | 4 | 0 | 0 | 0 | 0 | 0 | 0 | 0 | 0 | 1 | 1 | 0 | 0 | 2 | 1 | 3 |
| | 5 | 0 | 0 | 1 | 1 | 2 | 0 | 0 | 1 | 1 | 2 | 0 | 0 | 1 | 1 | 2 |
| | 6 | 0 | 0 | 0 | 0 | 0 | 0 | 0 | 0 | 0 | 0 | 0 | 0 | 0 | 0 | 0 |
| | 7 | 0 | 0 | 0 | 0 | 0 | 0 | 0 | 1 | 0 | 1 | 0 | 0 | 1 | 2 | 3 |
| | 8 | 0 | 0 | 0 | 1 | 1 | 0 | 0 | 0 | 1 | 1 | 0 | 0 | 2 | 1 | 3 |
| 6 | 1 | 0 | 0 | 0 | 0 | 0 | 0 | 0 | 0 | 0 | 0 | 0 | 0 | 0 | 0 | 0 |
| (CIA + | 2 | 0 | 0 | 0 | 0 | 0 | 0 | 0 | 0 | 0 | 0 | 0 | 0 | 1 | 0 | 1 |
| Compound A 3 | 3 | 0 | 0 | 0 | 0 | 0 | 0 | 0 | 0 | 0 | 0 | 0 | 0 | 0 | 0 | 0 |
| mg/kg) | 4 | 0 | 0 | 0 | 0 | 0 | 0 | 0 | 0 | 0 | 0 | 0 | 0 | 0 | 0 | 0 |
| | 5 | 0 | 0 | 0 | 0 | 0 | 0 | 0 | 1 | 0 | 1 | 0 | 0 | 1 | 2 | 3 |
| | 6 | 0 | 0 | 0 | 0 | 0 | 0 | 0 | 0 | 0 | 0 | 0 | 0 | 1 | 1 | 2 |
| | 7 | 0 | 0 | 0 | 0 | 0 | 0 | 0 | 0 | 0 | 0 | 0 | 0 | 0 | 0 | 0 |
| | 8 | 0 | 0 | 1 | 1 | 2 | 0 | 0 | 1 | 2 | 3 | 0 | 0 | 1 | 1 | 2 |
| 7 | 1 | 0 | 0 | 0 | 0 | 0 | 0 | 0 | 0 | 0 | 0 | 0 | 0 | 1 | 1 | 2 |
| (CIA + | 2 | 0 | 0 | 0 | 0 | 0 | 0 | 0 | 0 | 0 | 0 | 0 | 0 | 0 | 0 | 0 |
| Compound A 10 | 3 | 0 | 0 | 0 | 0 | 0 | 0 | 0 | 1 | 0 | 1 | 0 | 0 | 1 | 0 | 1 |
| mg/kg) | 4 | 0 | 0 | 0 | 1 | 1 | 0 | 0 | 0 | 2 | 2 | 0 | 0 | 2 | 2 | 4 |
| | 5 | 0 | 1 | 1 | 1 | 3 | 0 | 0 | 1 | 1 | 2 | 0 | 0 | 2 | 1 | 3 |
| | 6 | 0 | 0 | 0 | 0 | 0 | 0 | 0 | 0 | 0 | 0 | 0 | 0 | 0 | 0 | 0 |
| | 7 | 0 | 0 | 0 | 0 | 0 | 0 | 0 | 0 | 0 | 0 | 0 | 0 | 0 | 0 | 0 |
| | 8 | 0 | 0 | 0 | 0 | 0 | 0 | 0 | 0 | 0 | 0 | 0 | 0 | 0 | 0 | 0 |
| | | Day 18 | | | | | Day 19 | | | | | Day 20 | | | | |
| | | | | left | right | | | | left | right | | | | left | right | |
| | | left | right | hind | hind | | left | right | hind | hind | | left | right | hind | hind | |
| Group | ID | forelimb | forelimb | limb | limb | score | forelimb | forelimb | limb | limb | score | forelimb | forelimb | limb | limb | score |
| 1 | 1 | 0 | 0 | 0 | 0 | 0 | 0 | 0 | 0 | 0 | 0 | 0 | 0 | 0 | 0 | 0 |
| (No CIA + | 2 | 0 | 0 | 0 | 0 | 0 | 0 | 0 | 0 | 0 | 0 | 0 | 0 | 0 | 0 | 0 |
| Vehicle) | 3 | 0 | 0 | 0 | 0 | 0 | 0 | 0 | 0 | 0 | 0 | 0 | 0 | 0 | 0 | 0 |
| | 4 | 0 | 0 | 0 | 0 | 0 | 0 | 0 | 0 | 0 | 0 | 0 | 0 | 0 | 0 | 0 |
| | 5 | 0 | 0 | 0 | 0 | 0 | 0 | 0 | 0 | 0 | 0 | 0 | 0 | 0 | 0 | 0 |
| | 6 | 0 | 0 | 0 | 0 | 0 | 0 | 0 | 0 | 0 | 0 | 0 | 0 | 0 | 0 | 0 |
| | 7 | 0 | 0 | 0 | 0 | 0 | 0 | 0 | 0 | 0 | 0 | 0 | 0 | 0 | 0 | 0 |
| | 8 | 0 | 0 | 0 | 0 | 0 | 0 | 0 | 0 | 0 | 0 | 0 | 0 | 0 | 0 | 0 |
| 2 | 1 | 4 | 4 | 4 | 4 | 16 | 4 | 4 | 4 | 4 | 16 | 4 | 4 | 4 | 4 | 16 |
| (CIA + | 2 | 1 | 1 | 4 | 4 | 10 | 1 | 1 | 4 | 4 | 10 | 2 | 1 | 4 | 4 | 11 |
| Vehicle) | 3 | 4 | 4 | 4 | 4 | 16 | 4 | 4 | 4 | 4 | 16 | 4 | 4 | 4 | 4 | 16 |
| | 4 | 4 | 4 | 4 | 4 | 16 | 4 | 4 | 4 | 4 | 16 | 4 | 4 | 4 | 4 | 16 |
| | 5 | 1 | 2 | 4 | 4 | 11 | 1 | 2 | 4 | 4 | 11 | 1 | 2 | 4 | 4 | 11 |
| | 6 | 1 | 1 | 4 | 4 | 10 | 1 | 1 | 4 | 4 | 10 | 1 | 1 | 4 | 4 | 10 |
| | 7 | 1 | 1 | 4 | 4 | 10 | 1 | 1 | 4 | 4 | 10 | 1 | 1 | 4 | 4 | 10 |
| | 8 | 1 | 1 | 4 | 4 | 10 | 1 | 1 | 4 | 4 | 10 | 1 | 1 | 4 | 4 | 10 |
| 3 | 1 | 0 | 0 | 0 | 0 | 0 | 0 | 0 | 0 | 0 | 0 | 0 | 0 | 0 | 0 | 0 |
| (CIA+ | 2 | 0 | 0 | 0 | 0 | 0 | 0 | 0 | 0 | 0 | 0 | 0 | 0 | 0 | 0 | 0 |
| Dexamethasone) | 3 | 0 | 0 | 0 | 0 | 0 | 0 | 0 | 0 | 0 | 0 | 0 | 0 | 0 | 0 | 0 |
| | 4 | 0 | 0 | 0 | 0 | 0 | 0 | 0 | 0 | 0 | 0 | 0 | 0 | 0 | 0 | 0 |
| | 5 | 0 | 0 | 0 | 0 | 0 | 0 | 0 | 0 | 0 | 0 | 0 | 0 | 0 | 0 | 0 |
| | 6 | 0 | 0 | 0 | 0 | 0 | 0 | 0 | 0 | 0 | 0 | 0 | 0 | 0 | 0 | 0 |
| | 7 | 0 | 0 | 0 | 0 | 0 | 0 | 0 | 0 | 0 | 0 | 0 | 0 | 0 | 0 | 0 |
| | 8 | 0 | 0 | 0 | 0 | 0 | 0 | 0 | 0 | 0 | 0 | 0 | 0 | 0 | 0 | 0 |
| 4 | 1 | 0 | 0 | 4 | 4 | 8 | 1 | 0 | 4 | 4 | 9 | 1 | 0 | 4 | 4 | 9 |
| (CIA + | 2 | 0 | 0 | 4 | 4 | 8 | 1 | 1 | 4 | 4 | 10 | 2 | 1 | 4 | 4 | 11 |
| GSK620) | 3 | 0 | 0 | 4 | 4 | 8 | 0 | 0 | 3 | 3 | 6 | 0 | 0 | 3 | 3 | 6 |
| | 4 | 0 | 0 | 4 | 4 | 8 | 1 | 0 | 4 | 4 | 9 | 1 | 0 | 4 | 4 | 9 |
| | 5 | 0 | 0 | 4 | 0 | 4 | 1 | 0 | 4 | 0 | 5 | 1 | 0 | 4 | 1 | 6 |
| | 6 | 0 | 0 | 3 | 3 | 6 | 0 | 1 | 3 | 3 | 7 | 0 | 1 | 3 | 3 | 7 |
| | 7 | 0 | 0 | 4 | 4 | 8 | 1 | 0 | 4 | 3 | 8 | 1 | 0 | 4 | 3 | 8 |
| | 8 | 0 | 0 | 4 | 4 | 8 | 1 | 0 | 3 | 3 | 7 | 1 | 0 | 3 | 3 | 7 |
| 5 | 1 | 0 | 0 | 0 | 0 | 0 | 0 | 0 | 0 | 0 | 0 | 0 | 0 | 0 | 0 | 0 |
| (CIA + | 2 | 0 | 0 | 2 | 3 | 5 | 0 | 0 | 3 | 3 | 6 | 0 | 0 | 3 | 3 | 6 |
| Compound | 3 | 0 | 0 | 4 | 3 | 7 | 0 | 0 | 4 | 4 | 8 | 0 | 0 | 4 | 4 | 8 |
| A 1 mg/kg) | 4 | 0 | 0 | 3 | 2 | 5 | 0 | 0 | 3 | 3 | 6 | 0 | 0 | 3 | 3 | 6 |
| | 5 | 0 | 0 | 3 | 3 | 6 | 0 | 0 | 3 | 3 | 6 | 0 | 0 | 3 | 3 | 6 |
| | 6 | 0 | 0 | 1 | 1 | 2 | 0 | 0 | 1 | 2 | 3 | 0 | 0 | 1 | 1 | 2 |
| | 7 | 0 | 0 | 2 | 3 | 5 | 0 | 0 | 3 | 3 | 6 | 0 | 0 | 3 | 3 | 6 |
| | 8 | 0 | 0 | 2 | 2 | 4 | 0 | 0 | 3 | 3 | 6 | 0 | 0 | 3 | 3 | 6 |
| 6 | 1 | 0 | 0 | 2 | 2 | 4 | 0 | 0 | 2 | 3 | 5 | 0 | 0 | 2 | 3 | 5 |
| (CIA + | 2 | 0 | 0 | 2 | 1 | 3 | 0 | 0 | 2 | 2 | 4 | 0 | 0 | 3 | 2 | 5 |
| Compound | 3 | 0 | 0 | 0 | 2 | 2 | 0 | 0 | 1 | 3 | 4 | 0 | 0 | 1 | 2 | 3 |
| A 3 mg/kg) | 4 | 0 | 0 | 0 | 2 | 2 | 0 | 0 | 1 | 2 | 3 | 0 | 0 | 1 | 2 | 3 |
| | 5 | 0 | 0 | 1 | 2 | 3 | 0 | 0 | 1 | 2 | 3 | 0 | 0 | 2 | 3 | 5 |
| | 6 | 0 | 0 | 1 | 1 | 2 | 0 | 0 | 1 | 2 | 3 | 0 | 0 | 1 | 2 | 3 |
| | 7 | 0 | 0 | 1 | 1 | 2 | 0 | 0 | 1 | 2 | 3 | 0 | 0 | 1 | 2 | 3 |
| | 8 | 0 | 0 | 1 | 1 | 2 | 0 | 0 | 1 | 2 | 3 | 0 | 0 | 1 | 2 | 3 |
| 7 | 1 | 0 | 0 | 1 | 1 | 2 | 0 | 0 | 1 | 2 | 3 | 0 | 0 | 1 | 2 | 3 |
| (CIA + | 2 | 0 | 0 | 3 | 3 | 6 | 0 | 0 | 3 | 3 | 6 | 0 | 0 | 3 | 3 | 6 |
| Compound | 3 | 0 | 0 | 0 | 2 | 2 | 0 | 0 | 0 | 2 | 2 | 0 | 0 | 1 | 2 | 3 |
| A 10 | 4 | 0 | 0 | 1 | 2 | 3 | 0 | 0 | 2 | 3 | 5 | 0 | 0 | 3 | 3 | 6 |
| mg/kg) | 5 | 0 | 0 | 1 | 1 | 2 | 1 | 0 | 2 | 2 | 5 | 0 | 0 | 2 | 2 | 4 |
| | 6 | 0 | 0 | 0 | 0 | 0 | 0 | 0 | 0 | 0 | 0 | 0 | 0 | 0 | 0 | 0 |
| | 7 | 0 | 0 | 2 | 2 | 4 | 1 | 0 | 2 | 2 | 5 | 1 | 0 | 2 | 2 | 5 |
| | 8 | 0 | 0 | 0 | 1 | 1 | 0 | 0 | 0 | 2 | 2 | 0 | 0 | 0 | 2 | 2 |

# Each animal actually had a different number, and the animal numbers are shown for simplicity as 1-8 for each group.

TABLE 20B
Average detailed clinical score

| | Day 0 | | | | | Day 6 | | | | |
|---|---|---|---|---|---|---|---|---|---|---|
| | | | left | right | | | | left | right | |
| | left | right | hind | hind | | left | right | hind | hind | |
| Group | forelimb | forelimb | limb | limb | score | forelimb | forelimb | limb | limb | score |
| 1 | 0.00 | 0.00 | 0.00 | 0.00 | 0.00 | 0.00 | 0.00 | 0.00 | 0.00 | 0.00 |
| (No CIA + | | | | | | | | | | |
| Vehicle) | | | | | | | | | | |
| 2 | 0.00 | 0.00 | 0.00 | 0.00 | 0.00 | 0.00 | 0.00 | 0.00 | 0.00 | 0.00 |
| (CIA + Vehicle) | | | | | | | | | | |
| 3 | 0.00 | 0.00 | 0.00 | 0.00 | 0.00 | 0.00 | 0.00 | 0.00 | 0.00 | 0.00 |
| (CIA + | | | | | | | | | | |
| Dexamethasone) | | | | | | | | | | |
| 4 | 0.00 | 0.00 | 0.00 | 0.00 | 0.00 | 0.00 | 0.00 | 0.00 | 0.00 | 0.00 |
| (CIA + | | | | | | | | | | |
| GSK620) | | | | | | | | | | |
| 5 | 0.00 | 0.00 | 0.00 | 0.00 | 0.00 | 0.00 | 0.00 | 0.00 | 0.00 | 0.00 |
| (CIA + | | | | | | | | | | |
| Compound A 1 | | | | | | | | | | |
| mg/kg) | | | | | | | | | | |
| 6 | 0.00 | 0.00 | 0.00 | 0.00 | 0.00 | 0.00 | 0.00 | 0.00 | 0.00 | 0.00 |
| (CIA + | | | | | | | | | | |
| Compound A 3 | | | | | | | | | | |
| mg/kg) | | | | | | | | | | |
| 7 | 0.00 | 0.00 | 0.00 | 0.00 | 0.00 | 0.00 | 0.00 | 0.00 | 0.00 | 0.00 |
| (CIA + | | | | | | | | | | |
| Compound A | | | | | | | | | | |
| 10 mg/kg) | | | | | | | | | | |
| | Day 12 | | | | | Day 13 | | | | |
| | | | left | right | | | | left | right | |
| | left | right | hind | hind | | left | right | hind | hind | |
| Group | forelimb | forelimb | limb | limb | score | forelimb | forelimb | limb | limb | score |
| 1 | 0.00 | 0.00 | 0.00 | 0.00 | 0.00 | 0 | 0 | 0 | 0 | 0 |
| (No CIA + | | | | | | | | | | |
| Vehicle) | | | | | | | | | | |
| 2 | 0.13 | 0.00 | 0.63 | 0.50 | 1.25 | 0 | 0 | 1 | 1 | 2 |
| (CIA + Vehicle) | | | | | | | | | | |
| 3 | 0.00 | 0.00 | 0.00 | 0.00 | 0.00 | 0 | 0 | 0 | 0 | 0 |
| (CIA + | | | | | | | | | | |
| Dexamethasone) | | | | | | | | | | |
| 4 | 0.00 | 0.00 | 0.00 | 0.00 | 0.00 | 0 | 0 | 0 | 0.125 | 0.125 |
| (CIA + GSK620) | | | | | | | | | | |
| 5 | | | | | | 0 | 0 | 0 | 0 | 0 |
| (CIA + | 0.00 | 0.25 | 0.13 | 0.00 | 0.38 | | | | | |
| Compound A 1 | | | | | | | | | | |
| mg/kg) | | | | | | | | | | |
| 6 | | | | | | 0 | 0 | 0 | 0 | 0 |
| (CIA + | 0.00 | 0.00 | 0.25 | 0.25 | 0.50 | | | | | |
| Compound A 3 | | | | | | | | | | |
| mg/kg) | | | | | | | | | | |
| 7 | | | | | | 0 | 0.125 | 0.125 | 0.25 | 0.5 |
| (CIA + | 0.00 | 0.00 | 0.00 | 0.13 | 0.13 | | | | | |
| Compound A 10 | | | | | | | | | | |
| mg/kg) | | | | | | | | | | |
| | Day 14 | | | | | Day 15 | | | | |
| | | | left | right | | | | left | right | |
| | left | right | hind | hind | | left | right | hind | hind | |
| Group | forelimb | forelimb | limb | limb | score | forelimb | forelimb | limb | limb | score |
| 1 | 0 | 0 | 0 | 0 | 0 | 0 | 0 | 0 | 0 | 0 |
| (No CIA + | | | | | | | | | | |
| Vehicle) | | | | | | | | | | |
| 2 | 1 | 0 | 2 | 2 | 5 | 1 | 1 | 3 | 3 | 7 |
| (CIA + Vehicle) | | | | | | | | | | |
| 3 | 0 | 0 | 0 | 0 | 0 | 0 | 0 | 0 | 0 | 0 |
| (CIA + | | | | | | | | | | |
| Dexamethasone) | | | | | | | | | | |
| 4 | 0 | 0 | 0.875 | 1.125 | 2 | 0 | 0 | 1.625 | 2 | 3.625 |
| (CIA + GSK620) | | | | | | | | | | |
| 5 | 0 | 0 | 1 | 0 | 1 | 0 | 0 | 1 | 1 | 2 |
| (CIA + | | | | | | | | | | |
| Compound A 1 | | | | | | | | | | |
| mg/kg) | | | | | | | | | | |
| 6 | 0 | 0 | 0 | 0 | 1 | 0 | 0 | 1 | 1 | 1 |
| (CIA + | | | | | | | | | | |
| Compound A 3 | | | | | | | | | | |
| mg/kg) | | | | | | | | | | |
| 7 | 0 | 0 | 0.25 | 0.375 | 0.625 | 0 | 0 | 0.75 | 0.5 | 1.25 |
| (CIA + | | | | | | | | | | |
| Compound A 10 | | | | | | | | | | |
| mg/kg) | | | | | | | | | | |
| | Day 18 | | | | | Day 19 | | | | |
| | | | left | right | | | | left | right | |
| | left | right | hind | hind | | left | right | hind | hind | |
| Group | forelimb | forelimb | limb | limb | score | forelimb | forelimb | limb | limb | score |
| 1 | 0.00 | 0.00 | 0.00 | 0.00 | 0.00 | 0.00 | 0.00 | 0.00 | 0.00 | 0.00 |
| (No CIA + | | | | | | | | | | |
| Vehicle) | | | | | | | | | | |
| 2 | 2.13 | 2.25 | 4.00 | 4.00 | 12.38 | 2.13 | 2.25 | 4.00 | 4.00 | 12.38 |
| (CIA + Vehicle) | | | | | | | | | | |
| 3 | 0.00 | 0.00 | 0.00 | 0.00 | 0.00 | 0.00 | 0.00 | 0.00 | 0.00 | 0.00 |
| (CIA + | | | | | | | | | | |
| Dexamethasone) | | | | | | | | | | |
| 4 | 0.00 | 0.00 | 3.88 | 3.38 | 7.25 | 0.75 | 0.25 | 3.63 | 3.00 | 7.63 |
| (CIA + GSK620) | | | | | | | | | | |
| 5 | 0.50 | 0.50 | 3.88 | 3.88 | 8.75 | 1.13 | 1.00 | 4.00 | 3.75 | 9.88 |
| (CIA + | | | | | | | | | | |
| Compound A 1 | | | | | | | | | | |
| mg/kg) | | | | | | | | | | |
| 6 | 0.00 | 0.88 | 2.50 | 2.25 | 5.63 | 0.00 | 1.13 | 2.38 | 2.88 | 6.38 |
| (CIA + | | | | | | | | | | |
| Compound A 3 | | | | | | | | | | |
| mg/kg) | | | | | | | | | | |
| 7 | 0.00 | 0.00 | 1.13 | \|0.88 | 2.00 | 0.00 | 0.25 | 1.63 | 1.75 | 3.63 |
| (CIA + | | | | | | | | | | |
| Compound A 10 | | | | | | | | | | |
| mg/kg) | | | | | | | | | | |

| | Day 21 | | | | |
|---|---|---|---|---|---|
| | | | left | right | |
| | left | right | hind | hind | |
| Group | forelimb | forelimb | limb | limb | score |
| 1 | 0.00 | 0.00 | 0.00 | 0.00 | 0.00 |
| (No CIA + | | | | | |
| Vehicle) | | | | | |
| 2 | 2.25 | 2.25 | 4.00 | 4.00 | 12.50 |
| (CIA + Vehicle) | | | | | |
| 3 | 0.00 | 0.00 | 0.00 | 0.00 | 0.00 |
| (CIA + | | | | | |
| Dexamethasone) | | | | | |
| 4 | 0.88 | 0.25 | 3.63 | 3.13 | 7.88 |
| (CIA + GSK620) | | | | | |
| 5 | 1.00 | 1.00 | 4.00 | 3.75 | 9.75 |
| (CIA + | | | | | |
| Compound A 1 | | | | | |
| mg/kg) | | | | | |
| 6 | 0.00 | 1.25 | 2.50 | 2.75 | 6.50 |
| (CIA + | | | | | |
| Compound A 3 | | | | | |
| mg/kg) | | | | | |
| 7 | 0.00 | 0.13 | 1.88 | 1.63 | 3.63 |
| (CIA + | | | | | |
| Compound A 10 | | | | | |
| mg/kg) | | | | | |

TABLE 20C
Overall clinical scores

| | | Overall Clinical Score | | | | | | | | |
|---|---|---|---|---|---|---|---|---|---|---|
| Group | # | Day 0 | Day 6 | Day 12 | Day 13 | Day 14 | Day 15 | Day 18 | Day 19 | Day 21 |
| 1 | 1 | 0 | 0 | 0 | 0 | 0 | 0 | 0 | 0 | 0 |
| (No CIA + Vehicle) | 2 | 0 | 0 | 0 | 0 | 0 | 0 | 0 | 0 | 0 |
| | 3 | 0 | 0 | 0 | 0 | 0 | 0 | 0 | 0 | 0 |
| | 4 | 0 | 0 | 0 | 0 | 0 | 0 | 0 | 0 | 0 |
| | 5 | 0 | 0 | 0 | 0 | 0 | 0 | 0 | 0 | 0 |
| | 6 | 0 | 0 | 0 | 0 | 0 | 0 | 0 | 0 | 0 |
| | 7 | 0 | 0 | 0 | 0 | 0 | 0 | 0 | 0 | 0 |
| | 8 | 0 | 0 | 0 | 0 | 0 | 0 | 0 | 0 | 0 |
| 2 | 1 | 0 | 0 | 4 | 5 | 8 | 9 | 16 | 16 | 16 |
| (CIA + Vehicle) | 2 | 0 | 0 | 0 | 0 | 3 | 6 | 10 | 10 | 11 |
| | 3 | 0 | 0 | 0 | 0 | 0 | 4 | 16 | 16 | 16 |
| | 4 | 0 | 0 | 6 | 10 | 13 | 16 | 16 | 16 | 16 |
| | 5 | 0 | 0 | 0 | 0 | 4 | 8 | 11 | 11 | 11 |
| | 6 | 0 | 0 | 0 | 0 | 0 | 3 | 10 | 10 | 10 |
| | 7 | 0 | 0 | 0 | 3 | 5 | 7 | 10 | 10 | 10 |
| | 8 | 0 | 0 | 0 | 1 | 3 | 4 | 10 | 10 | 10 |
| 3 | 1 | 0 | 0 | 0 | 0 | 0 | 0 | 0 | 0 | 0 |
| (CIA + | 2 | 0 | 0 | 0 | 0 | 0 | 0 | 0 | 0 | 0 |
| Dexamethasone) | 3 | 0 | 0 | 0 | 0 | 0 | 0 | 0 | 0 | 0 |
| | 4 | 0 | 0 | 0 | 0 | 0 | 0 | 0 | 0 | 0 |
| | 5 | 0 | 0 | 0 | 0 | 0 | 0 | 0 | 0 | 0 |
| | 6 | 0 | 0 | 0 | 0 | 0 | 0 | 0 | 0 | 0 |
| | 7 | 0 | 0 | 0 | 0 | 0 | 0 | 0 | 0 | 0 |
| | 8 | 0 | 0 | 0 | 0 | 0 | 0 | 0 | 0 | 0 |
| 4 | 1 | 0 | 0 | 0 | 0 | 3 | 6 | 8 | 9 | 9 |
| (CIA + GSK620) | 2 | 0 | 0 | 0 | 0 | 2 | 6 | 8 | 10 | 11 |
| | 3 | 0 | 0 | 0 | 0 | 0 | 0 | 8 | 6 | 6 |
| | 4 | 0 | 0 | 0 | 1 | 3 | 4 | 8 | 9 | 9 |
| | 5 | 0 | 0 | 0 | 0 | 0 | 1 | 4 | 5 | 6 |
| | 6 | 0 | 0 | 0 | 0 | 0 | 2 | 6 | 7 | 7 |
| | 7 | 0 | 0 | 0 | 0 | 4 | 6 | 8 | 8 | 8 |
| | 8 | 0 | 0 | 0 | 0 | 4 | 4 | 8 | 7 | 7 |
| 5 | 1 | 0 | 0 | 0 | 0 | 0 | 0 | 0 | 0 | 0 |
| (CIA + Compound | 2 | 0 | 0 | 0 | 0 | 0 | 0 | 5 | 6 | 6 |
| A 1 mg/kg) | 3 | 0 | 0 | 0 | 0 | 2 | 4 | 7 | 8 | 8 |
| | 4 | 0 | 0 | 0 | 0 | 1 | 3 | 5 | 6 | 6 |
| | 5 | 0 | 0 | 1 | 2 | 2 | 2 | 6 | 6 | 6 |
| | 6 | 0 | 0 | 0 | 0 | 0 | 0 | 2 | 3 | 2 |
| | 7 | 0 | 0 | 0 | 0 | 1 | 3 | 5 | 6 | 6 |
| | 8 | 0 | 0 | 0 | 1 | 1 | 3 | 4 | 6 | 6 |
| 6 | 1 | 0 | 0 | 0 | 0 | 0 | 0 | 4 | 5 | 5 |
| (CIA + Compound | 2 | 0 | 0 | 0 | 0 | 0 | 1 | 3 | 4 | 5 |
| A 3 mg/kg) | 3 | 0 | 0 | 0 | 0 | 0 | 0 | 2 | 4 | 3 |
| | 4 | 0 | 0 | 0 | 0 | 0 | 0 | 2 | 3 | 3 |
| | 5 | 0 | 0 | 0 | 0 | 1 | 3 | 3 | 3 | 5 |
| | 6 | 0 | 0 | 0 | 0 | 0 | 2 | 2 | 3 | 3 |
| | 7 | 0 | 0 | 0 | 0 | 0 | 0 | 2 | 3 | 3 |
| | 8 | 0 | 0 | 1 | 2 | 3 | 2 | 2 | 3 | 3 |
| 7 | 1 | 0 | 0 | 0 | 0 | 0 | 2 | 2 | 3 | 3 |
| (CIA + Compound | 2 | 0 | 0 | 0 | 0 | 0 | 0 | 6 | 6 | 6 |
| A 10 mg/kg) | 3 | 0 | 0 | 0 | 0 | 1 | 1 | 2 | 2 | 3 |
| | 4 | 0 | 0 | 0 | 1 | 2 | 4 | 3 | 5 | 6 |
| | 5 | 0 | 0 | 2 | 3 | 2 | 3 | 2 | 5 | 4 |
| | 6 | 0 | 0 | 0 | 0 | 0 | 0 | 0 | 0 | 0 |
| | 7 | 0 | 0 | 0 | 0 | 0 | 0 | 4 | 5 | 5 |
| | 8 | 0 | 0 | 0 | 0 | 0 | 0 | 1 | 2 | 2 |

# Each animal actually had a different number, and the animal numbers are shown for simplicity as 1-8 for each group.

TABLE 20D
Average overall clinical score

| | | | | Day | | Day | Day | Day | Day |
|---|---|---|---|---|---|---|---|---|---|
| Group | Day 0 | Day 6 | Day 12 | 13 | Day 14 | 15 | 18 | 19 | 21 |
| 1 | 0 | 0 | 0 | 0 | 0 | 0 | 0 | 0 | 0 |
| (No CIA + | | | | | | | | | |
| Vehicle) | | | | | | | | | |
| 2 | 0 | 0 | 1 | 2 | 5 | 7 | 12 | 12 | 13 |
| (CIA + Vehicle) | | | | | | | | | |
| 3 | 0 | 0 | 0 | 0 | 0 | 0 | 0 | 0 | 0 |
| (CIA + | | | | | | | | | |
| Dexamethasone) | | | | | | | | | |
| 4 | 0 | 0 | 0 | 0 | 2 | 4 | 7 | 8 | 8 |
| (CIA + GSK620) | | | | | | | | | |
| 5 | 0 | 0 | 0 | 0 | 1 | 2 | 4 | 5 | 5 |
| (CIA + | | | | | | | | | |
| Compound A 1 | | | | | | | | | |
| mg/kg) | | | | | | | | | |
| 6 | 0 | 0 | 0 | 0 | 1 | 1 | 3 | 4 | 4 |
| (CIA + | | | | | | | | | |
| Compound A 3 | | | | | | | | | |
| mg/kg) | | | | | | | | | |
| 7 | 0 | 0 | 0 | 1 | 1 | 1 | 3 | 4 | 4 |
| (CIA + | | | | | | | | | |
| Compound A 10 | | | | | | | | | |
| mg/kg) | | | | | | | | | |

TABLE 20E
Clinical observations

| | | Clinical observations | | | | | | | | | |
|---|---|---|---|---|---|---|---|---|---|---|---|
| | | | | | | | | | Day | Day | |
| Group | # | Day 12 | Day 13 | Day 14 | Day 15 | Day 16 | Day 17 | Day 18 | 19 | 20 | Day 21 |
| 1 | 1 | — | — | — | — | — | — | — | — | — | — |
| (No CIA + | 2 | — | — | — | — | — | — | — | — | — | — |
| Vehicle) | 3 | — | — | — | — | — | — | — | — | — | — |
| | 4 | — | — | — | — | — | — | — | — | — | — |
| | 5 | — | — | — | — | — | — | — | — | — | — |
| | 6 | — | — | — | — | — | — | — | — | — | — |
| | 7 | — | — | — | — | — | — | — | — | — | — |
| | 8 | — | — | — | — | — | — | — | — | — | — |
| 2 | 1 | — | — | — | — | — | — | — | — | — | — |
| (CIA + | 2 | — | — | — | — | — | — | — | — | — | — |
| Vehicle) | 3 | — | — | — | — | — | — | — | — | — | — |
| | 4 | — | — | — | — | — | — | — | — | — | — |
| | 5 | — | — | — | — | — | — | — | — | — | — |
| | 6 | — | — | — | — | — | — | — | — | — | — |
| | 7 | — | — | — | — | — | — | — | — | — | — |
| | 8 | — | — | — | — | — | — | — | — | — | — |
| 3 | 1 | — | — | — | — | — | — | — | — | — | — |
| (CIA + | 2 | — | — | — | — | — | — | — | — | — | — |
| Dexa- | 3 | — | — | — | — | — | — | — | — | — | — |
| methasone) | 4 | — | — | — | — | — | — | — | — | — | — |
| | 5 | — | — | — | — | — | — | — | — | — | — |
| | 6 | — | — | — | — | — | — | — | — | — | — |
| | 7 | — | — | — | — | — | — | — | — | — | — |
| | 8 | — | — | — | — | — | — | — | — | — | — |
| 4 | 1 | — | — | — | — | — | — | — | — | — | — |
| (CIA + | 2 | — | — | — | — | — | — | — | — | — | — |
| GSK620) | 3 | — | — | — | — | — | — | — | — | — | — |
| | 4 | — | — | — | — | — | — | — | — | — | — |
| | 5 | — | — | — | — | — | — | — | — | — | — |
| | 6 | — | — | — | — | — | — | — | — | — | — |
| | 7 | — | — | — | — | — | — | — | — | — | — |
| | 8 | — | — | — | — | — | — | — | — | — | — |
| 5 | 1 | — | — | — | — | — | — | — | — | — | — |
| (CIA + | 2 | — | — | — | — | — | — | — | — | — | — |
| Com- | 3 | — | — | — | — | — | — | — | — | — | — |
| pound A 1 | 4 | — | — | — | — | Diarrhea | — | — | — | — | — |
| mg/kg) | 5 | — | — | — | — | Diarrhea | — | — | — | — | — |
| | 6 | — | — | — | — | — | — | — | — | — | — |
| | 7 | — | — | — | — | — | — | — | — | — | — |
| | 8 | — | — | — | — | — | — | — | — | — | — |
| 6 | 1 | — | — | Diarrhea | — | — | — | — | — | — | Diar- |
| (CIA + | | | | | | | | | | | rhea |
| Com- | 2 | — | Diarrhea | — | — | — | — | — | — | — | — |
| pound A 3 | 3 | — | — | — | — | — | — | — | — | — | — |
| mg/kg) | 4 | — | — | — | — | — | — | — | — | — | — |
| | 5 | — | — | — | Diarrhea | Diarrhea | — | — | — | — | — |
| | 6 | — | — | — | — | — | — | — | — | — | — |
| | 7 | — | — | — | — | — | — | — | — | — | — |
| | 8 | — | — | Diarrhea | — | — | — | — | — | — | — |
| 7 | 1 | Diarrhea | Diarrhea | Diarrhea | Diarrhea | Diarrhea | Diarrhea | Diarrhea | — | — | — |
| (CIA + | 2 | — | — | — | — | — | — | — | — | — | — |
| Com- | 3 | Diarrhea | — | — | — | — | — | — | — | — | — |
| pound A 10 | 4 | — | — | — | — | — | — | — | — | — | — |
| mg/kg) | 5 | — | — | — | — | — | — | — | — | — | — |
| | 6 | — | — | — | — | — | — | — | — | — | — |
| | 7 | — | Diarrhea | Diarrhea | — | Diarrhea | — | — | — | — | — |
| | 8 | — | — | Diarrhea | Diarrhea | Diarrhea | — | — | — | — | — |

# Each animal actually had a different number, and the animal numbers are shown for simplicity as 1-8 for each group.

TABLE 21
Compound A inhibition relative to GSK620

| | Percent Inhibition | Percent Inhibition |
|---|---|---|
| | in clinical | in paw volume |
| | score (Mean ± SD) | (Mean ± SD) |
| Compound A (1 mg/kg) | 60 ± 21 | 58 ± 23 |
| Compound A (3 mg/kg) | 70 ± 8 | 75 ± 12 |
| GSK620 (10 mg/kg) | 37 ± 14 | 28 ± 21 |

### Example 119—Tables—Part B: IgG1 Measurements

TABLE 22A
Rat anti-collagen IgG1 levels

| | | | | | | Rat Anti- | | |
|---|---|---|---|---|---|---|---|---|
| | | | | | | Collagen | | |
| | | | | | | IgG1 | | Final Rat Anti- |
| | | | | Average | | Antibody | | Collagen IgG1 |
| | | | | OD450 | Calculated | Con.(units/ | | Antibody |
| Group | ID | OD 450 nm | | nm | O.D. | ml) | Fold | Con.(units/ml) |
| 1 | 1 | 0.0576 | 0.0577 | 0.0577 | 0.0080 | 0.60 | 2 | 1.19 |
| (No CIA + | 2 | 0.0544 | 0.0580 | 0.0562 | 0.0066 | 0.49 | 2 | 0.98 |
| Vehicle) | 3 | 0.0557 | 0.0581 | 0.0569 | 0.0073 | 0.54 | 2 | 1.08 |
| | 4 | 0.0753 | 0.0707 | 0.0730 | 0.0234 | 1.79 | 2 | 3.58 |
| | 5 | 0.0649 | 0.0669 | 0.0659 | 0.0163 | 1.22 | 2 | 2.45 |
| | 6 | 0.0622 | 0.0560 | 0.0591 | 0.0095 | 0.70 | 2 | 1.41 |
| | 7 | 0.0654 | 0.0671 | 0.0663 | 0.0166 | 1.25 | 2 | 2.50 |
| | 8 | 0.0617 | 0.0618 | 0.0618 | 0.0121 | 0.90 | 2 | 1.81 |
| 2 | 1 | 0.2248 | 0.2282 | 0.2265 | 0.1769 | 16.15 | 2000 | 32306.30 |
| (CIA + | 2 | 0.1409 | 0.1352 | 0.1381 | 0.0884 | 7.49 | 2000 | 14970.83 |
| Vehicle) | 3 | 0.1044 | 0.1020 | 0.1032 | 0.0536 | 4.34 | 2000 | 8675.25 |
| | 4 | 0.0908 | 0.0893 | 0.0901 | 0.0404 | 3.20 | 2000 | 6404.64 |
| | 5 | 0.2026 | 0.1990 | 0.2008 | 0.1512 | 13.55 | 2000 | 27093.89 |
| | 6 | 0.1050 | 0.1069 | 0.1060 | 0.0563 | 4.58 | 2000 | 9158.20 |
| | 7 | 0.1645 | 0.1660 | 0.1653 | 0.1156 | 10.06 | 2000 | 20113.89 |
| | 8 | 0.1565 | 0.1590 | 0.1578 | 0.1081 | 9.34 | 2000 | 18677.72 |
| 3 | 1 | 0.1744 | 0.1758 | 0.1751 | 0.1255 | 11.01 | 2000 | 22019.88 |
| (CIA+ | 2 | 0.0834 | 0.0830 | 0.0832 | 0.0336 | 2.63 | 2000 | 5250.27 |
| Dexamethasone) | 3 | 0.0587 | 0.0580 | 0.0584 | 0.0087 | 0.65 | 2000 | 1296.14 |
| | 4 | 0.0648 | 0.0682 | 0.0665 | 0.0169 | 1.27 | 2000 | 2541.14 |
| | 5 | 0.1219 | 0.1189 | 0.1204 | 0.0708 | 5.87 | 2000 | 11736.83 |
| | 6 | 0.0778 | 0.0802 | 0.0790 | 0.0294 | 2.28 | 2000 | 4553.59 |
| | 7 | 0.0997 | 0.0981 | 0.0989 | 0.0493 | 3.96 | 2000 | 7925.50 |
| | 8 | 0.0970 | 0.0941 | 0.0956 | 0.0459 | 3.67 | 2000 | 7346.16 |
| 4 | 1 | 0.1048 | 0.1070 | 0.1059 | 0.0563 | 4.57 | 2000 | 9149.39 |
| (CIA + | 2 | 0.1158 | 0.1165 | 0.1162 | 0.0665 | 5.49 | 2000 | 10971.56 |
| GSK620) | 3 | 0.0510 | 0.0510 | 0.0510 | 0.0014 | 0.13 | 2000 | 258.62 |
| | 4 | 0.0597 | 0.0584 | 0.0591 | 0.0094 | 0.70 | 2000 | 1400.18 |
| | 5 | 0.1344 | 0.1422 | 0.1383 | 0.0887 | 7.51 | 2000 | 15017.25 |
| | 6 | 0.0652 | 0.0696 | 0.0674 | 0.0178 | 1.34 | 2000 | 2682.44 |
| | 7 | 0.1051 | 0.1085 | 0.1068 | 0.0572 | 4.65 | 2000 | 9308.00 |
| | 8 | 0.1186 | 0.1193 | 0.1190 | 0.0693 | 5.74 | 2000 | 11475.12 |
| 5 | 1 | 0.1083 | 0.0847 | 0.0965 | 0.0469 | 3.76 | 500 | 1877.50 |
| (CIA + | 2 | 0.1238 | 0.1010 | 0.1124 | 0.0628 | 5.15 | 500 | 2575.24 |
| Compound | 3 | 0.0516 | 0.0566 | 0.0541 | 0.0045 | 0.34 | 500 | 170.24 |
| A 1 | 4 | 0.0718 | 0.0958 | 0.0838 | 0.0342 | 2.68 | 500 | 1337.63 |
| mg/kg) | 5 | 0.0566 | 0.0546 | 0.0556 | 0.0060 | 0.45 | 500 | 223.60 |
| | 6 | 0.0720 | 0.0629 | 0.0675 | 0.0178 | 1.35 | 500 | 672.58 |
| | 7 | 0.0942 | 0.0898 | 0.0920 | 0.0424 | 3.37 | 500 | 1684.26 |
| | 8 | 0.0674 | 0.0791 | 0.0733 | 0.0236 | 1.81 | 500 | 903.85 |
| 6 | 1 | 0.0622 | 0.0519 | 0.0571 | 0.0074 | 0.55 | 500 | 276.19 |
| (CIA + | 2 | 0.0587 | 0.0524 | 0.0556 | 0.0059 | 0.44 | 500 | 221.80 |
| Compound | 3 | 0.0625 | 0.0659 | 0.0642 | 0.0146 | 1.09 | 500 | 545.78 |
| A 3 | 4 | 0.0915 | 0.0885 | 0.0900 | 0.0404 | 3.20 | 500 | 1599.04 |
| mg/kg) | 5 | 0.0696 | 0.0643 | 0.0670 | 0.0173 | 1.31 | 500 | 652.93 |
| | 6 | 0.0605 | 0.0503 | 0.0554 | 0.0058 | 0.43 | 500 | 216.42 |
| | 7 | 0.0735 | 0.0687 | 0.0711 | 0.0215 | 1.63 | 500 | 817.44 |
| | 8 | 0.0536 | 0.0578 | 0.0557 | 0.0061 | 0.45 | 500 | 227.20 |
| 7 | 1 | 0.0532 | 0.0508 | 0.0520 | 0.0024 | 0.20 | 500 | 97.82 |
| (CIA + | 2 | 0.0561 | 0.0559 | 0.0560 | 0.0063 | 0.48 | 500 | 238.02 |
| Compound | 3 | 0.0535 | 0.0508 | 0.0522 | 0.0025 | 0.21 | 500 | 102.88 |
| A 10 | 4 | 0.0562 | 0.0497 | 0.0530 | 0.0033 | 0.26 | 500 | 130.19 |
| mg/kg) | 5 | 0.0564 | 0.0481 | 0.0523 | 0.0026 | 0.21 | 500 | 106.27 |
| | 6 | 0.0589 | 0.0519 | 0.0554 | 0.0058 | 0.43 | 500 | 216.42 |
| | 7 | 0.0692 | 0.0552 | 0.0622 | 0.0126 | 0.94 | 500 | 468.93 |
| | 8 | 0.0618 | 0.0590 | 0.0604 | 0.0108 | 0.80 | 500 | 400.65 |

# Each animal actually had a different number, and the animal numbers are shown for simplicity as 1-8 for each group.

TABLE 22B
Average rat anti-collagen IgG1 levels

| Group | Mean | SEM |
|---|---|---|
| 1 (No CIA + Vehicle) | 1.87** | 0.32 |
| 2 (CIA + Vehicle) | 17175.09 | 3261.27 |
| 3 (CIA + Dexamethasone) | 7833.69* | 2333.42 |
| 4 (CIA + GSK620) | 7532.82* | 1904.87 |
| 5 (CIA + Compound A 1 mg/kg) | 1180.61** | 298.22 |
| 6 (CIA + Compound A 3 mg/kg) | 569.60** | 167.85 |
| 7 (CIA + Compound A 10 mg/kg) | 220.15** | 50.81 |

One-way ANOVA comparison to Group 2;
*p < 0.05;
**p < 0.01

### Example 119—Tables—Part C: Pharmacokinetics of Compound a in Lewis Rats (Oral)

TABLE 23A
Day 21 plasma concentration

| | | Plasma Concentration |
|---|---|---|
| Group | ID | (ng/mL) |
| 4 (CIA + GSK620) | 1 | 3230 |
| | 2 | 3450 |
| | 3 | 2540 |
| | 4 | 1590 |
| | 5 | 4170 |
| | 6 | 3070 |
| | 7 | 2740 |
| | 8 | 1760 |
| 5 (CIA + Compound A 1 mg/kg) | 1 | 13.0 |
| | 2 | 12.8 |
| | 3 | 14.5 |
| | 4 | 13.9 |
| | 5 | 14.3 |
| | 6 | 21.1 |
| | 7 | 13.9 |
| | 8 | 20.6 |
| 6 (CIA + Compound A 3 mg/kg) | 1 | 41.8 |
| | 2 | 55.8 |
| | 3 | 55.0 |
| | 4 | 68.8 |
| | 5 | 38.8 |
| | 6 | 43.7 |
| | 7 | 60.7 |
| | 8 | 31.0 |
| 7 (CIA + Compound A 10 mg/kg) | 1 | 232 |
| | 2 | 181 |
| | 3 | 240 |
| | 4 | 230 |
| | 5 | 264 |
| | 6 | 174 |
| | 7 | 255 |
| | 8 | 248 |

# Each animal actually had a different number, and the animal numbers are shown for simplicity as 1-8 for each group.

TABLE 23B
Day 21 average plasma concentration

| | Mean Plasma | | |
|---|---|---|---|
| | Concentration | SD | |
| Group | (ng/mL) | (ng/mL) | CV (%) |
| 4 (CIA + GSK620) | 2819 | 860 | 30.5 |
| 5 (CIA + Compound A 1 mg/kg) | 15.5 | 3.3 | 21.6 |
| 6 (CIA + Compound A 3 mg/kg) | 49.5 | 12.6 | 25.6 |
| 7 (CIA + Compound A 10 mg/kg) | 221 | 31 | 13.9 |

TABLE 23C
Plasma concentration over time after oral
administration on day 0 (3 mg/kg)

Concentration (ng/mL)

| Time (h) | Test Subject 1 | Test Subject 2 | Test Subject 3 |
|---|---|---|---|
| 0.25 | 28.7 | 12.9 | 8.85 |
| 0.5 | 39.3 | 25.4 | 19.5 |
| 1 | 78.4 | 40.1 | 34.7 |
| 2 | 109 | 66.0 | 62.7 |
| 4 | 112 | 77.9 | 80.7 |
| 6 | 123 | 77.1 | 82.6 |
| 8 | 119 | 76.1 | 74.4 |
| 24 | 35.6 | 14.1 | 17.3 |

TABLE 23D
Plasma concentration over time after oral
administration on day 21 (3 mg/kg)

Concentration (ng/mL)

| Time (h) | Test Subject 1 | Test Subject 2 | Test Subject 3 |
|---|---|---|---|
| 0.25 | 18.4 | 14.7 | 13.1 |
| 0.5 | 22.2 | 20.9 | 19.8 |
| 1 | 33.6 | 27.0 | 25.5 |
| 2 | 33.0 | 45.6 | 33.8 |
| 4 | 48.3 | 44.3 | 44.7 |
| 6 | 44.2 | 51.2 | 45.7 |
| 8 | 30.9 | 45.6 | 45.3 |
| 24 | 6.60 | 15.6 | 14.4 |

TABLE 23E
Average plasma concentration over time after
oral administration on day 0 (3 mg/kg)

| | Mean | SD | |
|---|---|---|---|
| Time | Concentration (ng/mL) | (ng/mL) | CV (%) |
| 0.25 | 16.8 | 10.5 | 62.4 |
| 0.5 | 28.1 | 10.2 | 36.2 |
| 1 | 51.1 | 23.8 | 46.7 |
| 2 | 79 | 26 | 32.6 |
| 4 | 90 | 19 | 21.0 |
| 6 | 94 | 25 | 26.6 |
| 8 | 90 | 25 | 28.1 |
| 24 | 22.3 | 11.6 | 51.9 |

TABLE 23F
Average plasma concentration over time after
oral administration on day 21 (3 mg/kg)

| | Mean | SD | |
|---|---|---|---|
| Time | Concentration (ng/mL) | (ng/mL) | CV (%) |
| 0.25 | 15.4 | 2.7 | 17.7 |
| 0.5 | 21.0 | 1.2 | 5.73 |
| 1 | 28.7 | 4.3 | 15.0 |
| 2 | 37.5 | 7.1 | 18.8 |
| 4 | 45.8 | 2.2 | 4.81 |
| 6 | 47.0 | 3.7 | 7.84 |
| 8 | 40.6 | 8.4 | 20.7 |
| 24 | 12.2 | 4.9 | 40.1 |

TABLE 23G
Compound A pharmacokinetic profiles in
CIA-induced animals on day 0 (3 mg/kg)

| | | Test | Test | Test |
|---|---|---|---|---|
| PK Parameters | Unit | Subject 1 | Subject 2 | Subject 3 |
| T_{1/2} | h | 9.19 | 7.03 | 7.60 |
| Tₘₐₓ | h | 6.00 | 4.00 | 6.00 |
| Cₘₐₓ | ng/mL | 123 | 77.9 | 82.6 |
| AUCₗₐₛₜ | h*ng/mL | 2070 | 1250 | 1264 |
| AUC_{Inf} | h*ng/mL | 2542 | 1393 | 1454 |
| AUC_%Extrap_obs | % | 18.6 | 10.3 | 13.1 |
| MRT_{Inf}_obs | h | 14.1 | 10.7 | 11.8 |
| AUCₗₐₛₜ/D | h*mg/mL | 690 | 417 | 421 |
| F | % | NA | NA | NA |

TABLE 23H
Compound A pharmacokinetic profiles in
CIA-induced animals on day 21 (3 mg/kg)

| | | Test | Test | Test |
|---|---|---|---|---|
| PK Parameters | Unit | Subject 1 | Subject 2 | Subject 3 |
| T_{1/2} | h | 6.79 | 10.3 | 9.68 |
| Tₘₐₓ | h | 4.00 | 6.00 | 6.00 |
| Cₘₐₓ | ng/mL | 48.3 | 51.2 | 45.7 |
| AUCₗₐₛₜ | h*ng/mL | 604 | 826 | 784 |
| AUC_{Inf} | h*ng/mL | 668 | 1059 | 985 |
| AUC_%Extrap_obs | % | 9.67 | 22.0 | 20.4 |
| MRT_{Inf}_obs | h | 10.1 | 15.5 | 14.9 |
| AUCₗₐₛₜ/D | h*mg/mL | 201 | 275 | 261 |
| F | % | NA | NA | NA |

TABLE 23I
Average Compound A pharmacokinetic profile
in CIA-induced animals on day 0 (3 mg/kg)

| PK Parameters | Unit | Mean | SD | CV (%) |
|---|---|---|---|---|
| T_{1/2} | h | 7.94 | 1.12 | 14.1 |
| Tₘₐₓ | h | 5.33 | 1.15 | 21.7 |
| Cₘₐₓ | ng/mL | 95 | 25 | 26.2 |
| AUCₗₐₛₜ | h*ng/mL | 1528 | 470 | 30.7 |
| AUC_{Inf} | h*ng/mL | 1796 | 647 | 36.0 |
| AUC_%Extrap_obs | % | 14.0 | 4.2 | 30.3 |
| MRT_{Inf}_obs | h | 12.2 | 1.7 | 14.2 |
| AUCₗₐₛₜ/D | h*mg/mL | 509 | 157 | 30.7 |
| F | % | NA | NA | NA |

TABLE 23J
Average Compound A pharmacokinetic profile
in CIA-induced animals on day 21 (3 mg/kg)

| PK Parameters | Unit | Mean | SD | CV (%) |
|---|---|---|---|---|
| T_{1/2} | h | 8.9 | 1.9 | 21.1 |
| Tₘₐₓ | h | 5.33 | 1.15 | 21.7 |
| Cₘₐₓ | ng/mL | 48.4 | 2.8 | 5.68 |
| AUCₗₐₛₜ | h*ng/mL | 738 | 118 | 16.0 |
| AUC_{lnf} | h*ng/mL | 904 | 208 | 23.0 |
| AUC_{—%Extrap}_obs | % | 17.3 | 6.7 | 38.6 |
| MRT_{lnf}_obs | h | 13.5 | 3.0 | 22.2 |
| AUCₗₐₛₜ/D | h*mg/mL | 246 | 39 | 16.0 |
| F | % | NA | NA | NA |

### Example 119—Tables—Part D: Histopathologic Data

TABLE 24
Observations in hind limbs from histopathologic analysis

| | Group 1 | Group 2 | Group 3 | Group 4 | Group 5 | Group 6 | Group 7 |
|---|---|---|---|---|---|---|---|
| CIA Induced? | — | Yes | Yes | Yes | Yes | Yes | Yes |
| Treatment | — | — | Dexamethasone | GSK620 | Compound | Compound | Compound |
| | | | | | A (1 | A (3 | A (10 |
| | | | | | mg/kg) | mg/kg) | mg/kg) |
| Number of | 8 | 8 | 8 | 8 | 8 | 8 | 8 |
| Animals in Group | | | | | | | |
| Number of | 8 | 8 | 8 | 8 | 8 | 8 | 8 |
| Animals | | | | | | | |
| Examined | | | | | | | |
| No Visible | 8 | 0 | 8 | 0 | 1 | 2 | 6 |
| Lesions | | | | | | | |
| Mixed cell | | | | | | | |
| inflammation | | | | | | | |
| Minimal | 0 | 0 | 0 | 0 | 1 | 0 | 0 |
| Mild | 0 | 0 | 0 | 0 | 2 | 2 | 1 |
| Moderate | 0 | 0 | 0 | 8 | 4 | 4 | 1 |
| Marked | 0 | 8 | 0 | 0 | 0 | 0 | 0 |
| Granulation | | | | | | | |
| tissue | | | | | | | |
| Minimal | 0 | 0 | 0 | 0 | 0 | 0 | 0 |
| Mild | 0 | 0 | 0 | 4 | 3 | 4 | 1 |
| Moderate | 0 | 0 | 0 | 4 | 3 | 2 | 0 |
| Marked | 0 | 8 | 0 | 0 | 0 | 0 | 0 |
| Increased eroded | | | | | | | |
| surface, bone | | | | | | | |
| Minimal | 0 | 0 | 0 | 1 | 0 | 2 | 1 |
| Mild | 0 | 0 | 0 | 4 | 3 | 3 | 0 |
| Moderate | 0 | 2 | 0 | 3 | 2 | 0 | 0 |
| Marked | 0 | 6 | 0 | 0 | 0 | 0 | 0 |
| Increased bone, | | | | | | | |
| periosteum | | | | | | | |
| Minimal | 0 | 0 | 0 | 0 | 1 | 2 | 0 |
| Mild | 0 | 0 | 0 | 6 | 4 | 4 | 0 |
| Moderate | 0 | 0 | 0 | 2 | 0 | 0 | 0 |
| Marked | 0 | 8 | 0 | 0 | 0 | 0 | 0 |
| Erosion/Ulcer, | | | | | | | |
| cartilaginous | | | | | | | |
| Minimal | 0 | 0 | 0 | 0 | 0 | 3 | 1 |
| Mild | 0 | 0 | 0 | 6 | 6 | 3 | 1 |
| Moderate | 0 | 3 | 0 | 2 | 1 | 0 | 0 |
| Marked | 0 | 5 | 0 | 0 | 0 | 0 | 0 |

### Example 120: Unilateral Urethral Obstruction in Animals Treated with a BET Inhibitor and Vehicle

The Unilateral Urethral Obstruction (UUO) model of renal fibrosis was used to assess the efficacy of oral delivery of Compound A at a 1 mg/mL concentration in rats. Compound A and control suspensions were prepared according to the procedures set out in Experimental Method A. Evaluation of the effect of Compound A compared to vehicle and controls in UUO in rats was undertaken in accordance with the protocol set out in Experimental Method E. Experimental groups for the study are outlined in Table 6. Prior to being randomly placed in treatment groups, animals were weighed. Once randomly assigned to treatment groups, on Day 0, animals in groups 2-4 were subjected to UUO surgery.

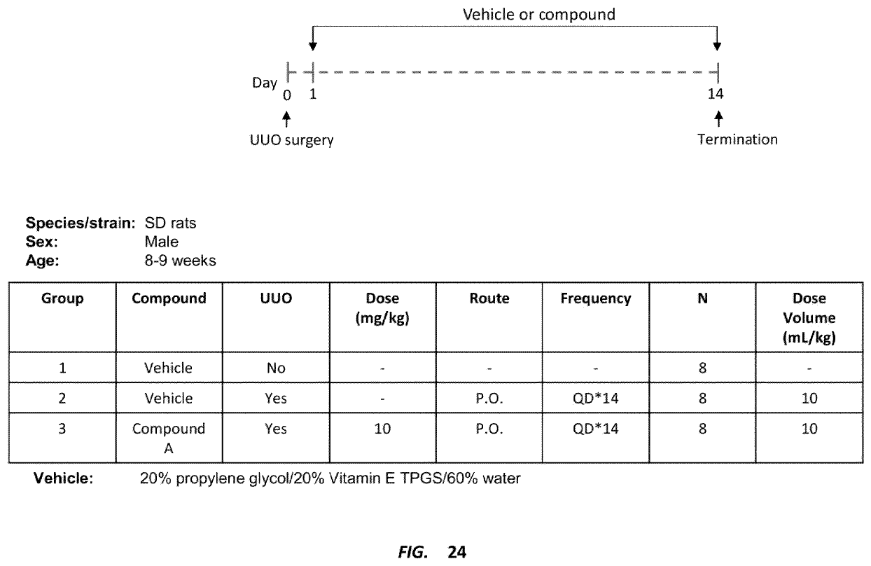

Starting on Day 0, animals in group 2 were administered daily doses of the vehicle for 14 days. Starting on Day 0, animals in group 3 were administered daily doses of Compound A (10 mg/kg) for 14 days. A design of the UUO study is depicted in FIG. 24.

Body weight of all animals was recorded daily for the 14-day period. Blind clinical nephropathy scores were conducted for all animals in the study. Assessments were recording according to the clinical scoring system summarized in Table 7. Glomerulosclerosis, interstitial nephritis, collagen fiber deposition, and nephropathy were each assessed for all experimental rats, and a total clinical score was determined for each rat. The scoring system represented the degree or percentage of lesions in the whole kidney. Clinical score assessments were performed on Day 14.

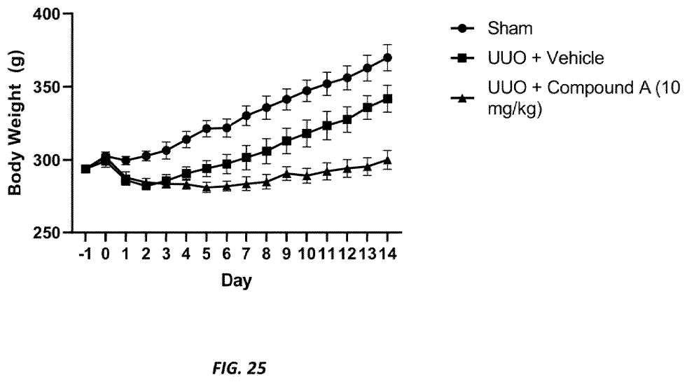

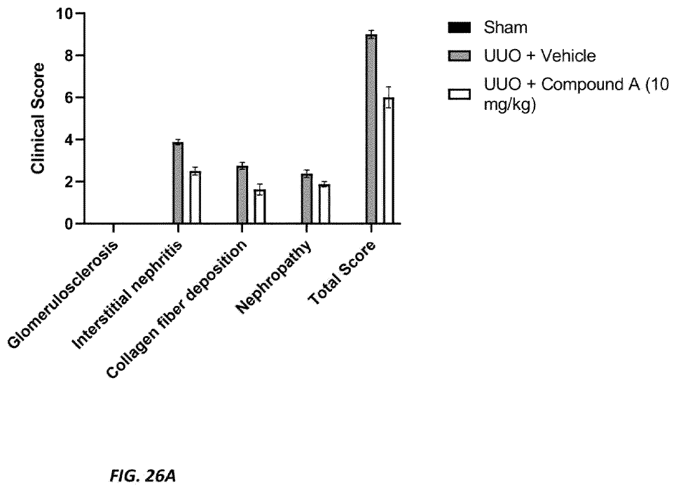

Animal body weight was measured daily over the course of the study (Table 25B; FIG. 25). UUO animals receiving no treatment (group 2 in Table 25A-25B; square shape in FIG. 25) exhibited a modest weight gain over the 14-day experimental period. In sham animals where UUO was not induced and no treatment was administered (group 1 in Tables 25A-25B; circle shape in FIG. 25), the increase in weight was more pronounced relative to in UUO animals receiving no treatment. Animals receiving Compound A (10 mg/kg; group 3 in Tables 25A-25B; triangle shape in FIG. 25) exhibited a slight weight loss over the course of the 14-day study before regaining their original weight at 14-days post-treatment.

Table 26 shows clinical observations assessed daily in rats from each experimental group. As can be seen, two sham rats and one UUO rat treated with Compound A each exhibited soft stools on just one study day. No UUO rats treated with vehicle exhibited clinical observations.

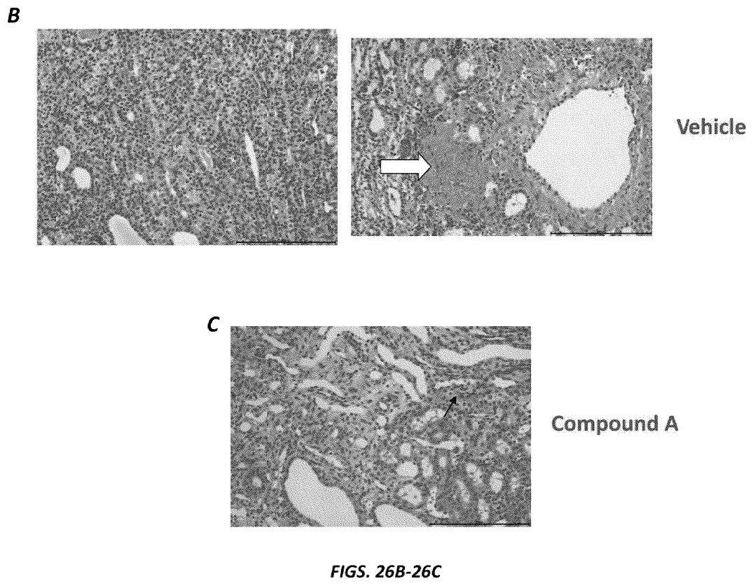

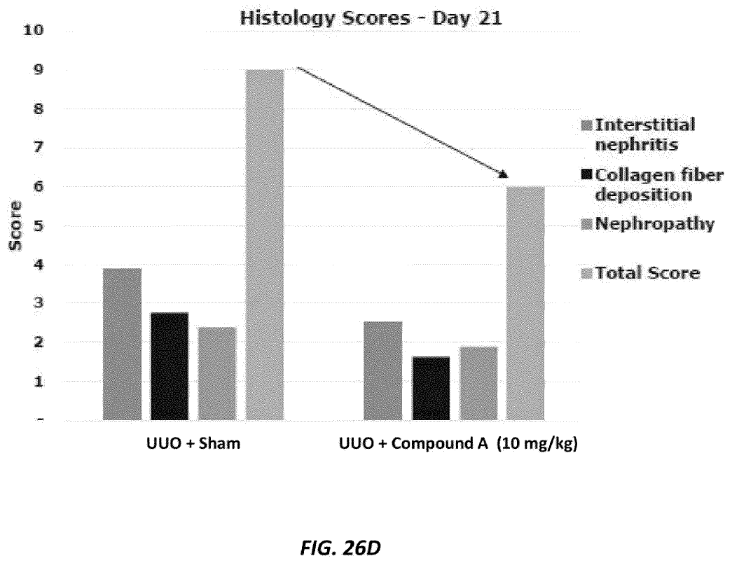

Table 27 and FIG. 26A depicts clinical histopathology scores for animals in the different treatment groups. As can be seen, treatment of UUO rats with Compound A exhibited reduced levels of interstitial nephritis, collagen fiber deposition, and nephropathy, as well as total clinical scores. Representative samples of pathology staining are shown in FIG. 26B (UUO rat with vehicle treatment) and FIG. 26C (UUO rat with Compound A treatment). UUO rats treated with vehicle (FIG. 26B) generally exhibited severe interstitial nephritis with extensive neutrophil and lymphocyte infiltration, degeneration, and necrosis in tubular epithelial cells, as well tubular dilatation. In FIG. 26B, the white arrow points to a large number of fibroblasts proliferating between renal tubules, with extensive collagen fiber deposition. UUO rats treated with Compound (A) (FIG. 26C) generally exhibited mild interstitial nephritis with a small amount of neutrophil and lymphocyte infiltration, degeneration of tubular epithelial cells, and tubular dilatation. In FIG. 26C, the black arrow points to fibroblasts proliferated slightly between renal tubules, with occasional deposition of collagen fibers. FIG. 26D illustrates the day 21 histology scores in UUO+Sham and UUO+Compound A (10 mg/kg) treated animals. Animals receiving Compound A exhibited a marked decrease in total histology score relative to sham treated animals, and also exhibited lower scores for interstitial nephritis, collagen fiber deposition, and nephropathy.

Table 28 depicts the pharmacokinetic profile of Compound A after oral administration in Lewis rats. Blood was sampled four hours after final treatment with Compound A, and the concentration of Compound A was measured. The mean plasma concentration of Compound A in rats was 201 ng/mL. The pharmacokinetic profile indicates that Compound A may be therapeutically effective with a single daily unit dose.

Data in Tables 29A and 29B show marked reductions in RNA levels and mean RNA levels respectively of various tissue biomarkers in rats of UUO Study. See observations below.

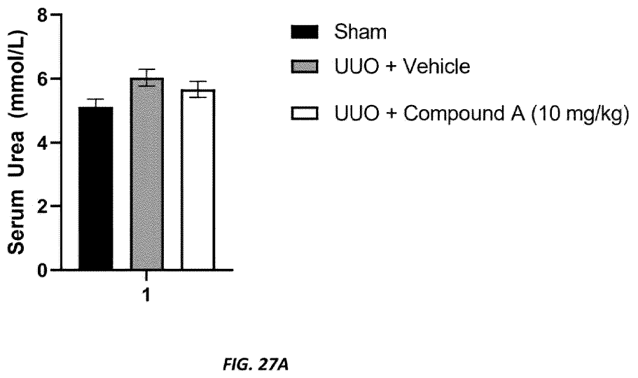

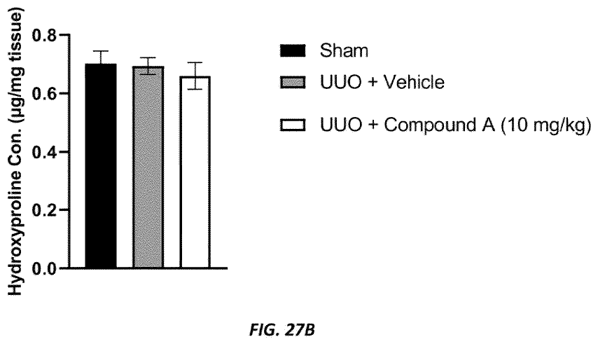

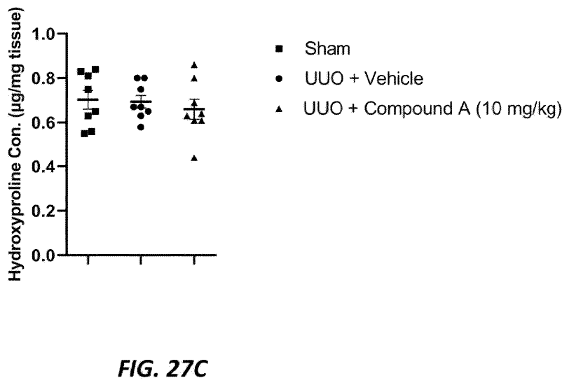

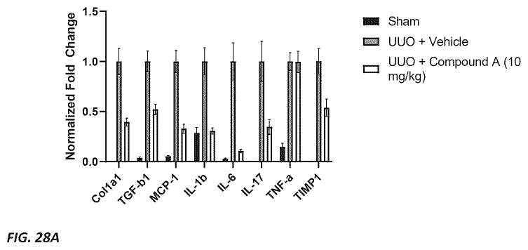

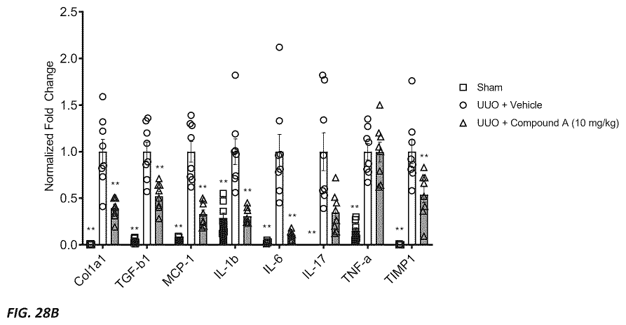

Tables 30A-30B and FIG. 27A depict serum urea levels in sham rats (black bar), UUO rats treated with vehicle (grey bar), and UUO rats treated with Compound A (white bar). No significant differences in serum urea levels between the three experimental groups were observed.

Table 31 and FIGS. 27B-27C depict hydroxyproline levels in sham rats (black bar in FIG. 27B; square shape in FIG. 27C), UUO rats treated with vehicle (grey bar in FIG. 27B; circle shape in FIG. 27C), and UUO rats treated with Compound A (white bar in FIG. 27B; triangle shape in FIG. 27C). No significant differences in hydroxyproline levels between the three experimental groups were observed.

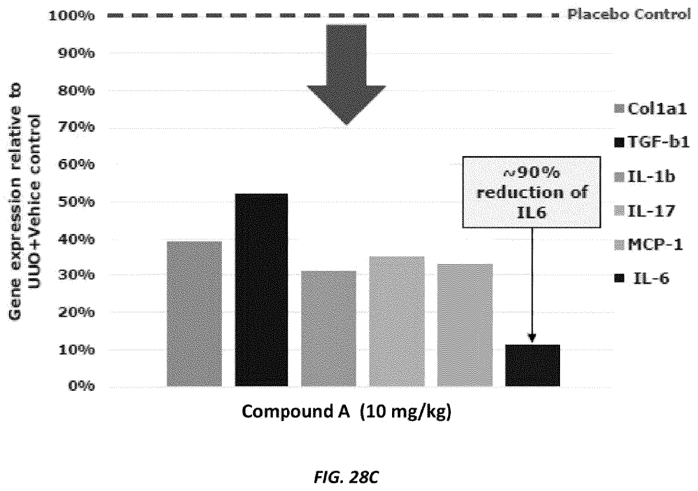

Tables 29A-29B and FIGS. 28A-28B show tissue RNA levels of various biomarkers (Col1a1, TGB-b1, MCP-1, IL-1 b, IL-6, IL-17, TNF-a, and Timp1) in sham rats (black bar in FIG. 28A; square shape in FIG. 28B), UUO rats treated with vehicle (grey bar in FIG. 28A; circle shape in FIG. 28B), and UUO rats treated with Compound A (white bar in FIG. 28A; triangle shape in FIG. 28B). Treatment of UUO rats with Compound A reduced levels of Col1a1, TGB-b1, MCP-1, IL-1b, IL-6, IL-17, and Timp1. Treatment with Compound A appeared to have no observable effect on TNF-a, which is surprising given the marked reduction seen with other biomarkers. FIG. 28C further illustrates gene expression levels of Col1a1, TGB-b1, IL-1 b, IL-17, MCP-1, and IL-6 relative to the vehicle control. Notably, there was a marked reduction in expression of all the genes relative to the vehicle control, and an approximately 90% reduction was observed in the expression of IL-6 in Compound A treated animals. To place this reduction in context, IL-6 is said to be a significant contributor to a continued inflammatory response in certain nephritis conditions.

In sum, Compound A 10 mg/kg PO QD treatment reduced the severity of all histological indicators of fibrosis: interstitial nephritis, collagen fiber deposition and overall nephropathy relative to UUO+Placebo control. Key inflammatory and pro-fibrotic biomarkers were attenuated by Compound A 10 mg/kg PO QD treatment. Expression of COL1A1 and TGF-b1 were markedly lower and correlated with less collagen fiber deposition in the kidney and improved histology outcomes. Notably as illustrated by FIG. 28C the expression of IL6 relative to UUO+Placebo control was greatly reduced (about 90%). It follows from the cytokine data generated in PBMCs, in the psoriasis model, and in the UUO model that the compounds disclosed herein, e.g., Compounds A, B, C, and D, are capable of amelioration or reducing inflammation in psoriasis and fibrosis, and may further ameliorate, reduce, or retard fibrosis with reduced collagen deposition as observed in the UUO model.

Based on these data, 10 mg/kg of Compound A ameliorates clinical symptoms associated with UUO.

### Example 120—Tables

TABLE 25A
Body Weight of Rats in UUO Study

| | | | Body Weight/g | | | | | | | | | | | | | | | |
|---|---|---|---|---|---|---|---|---|---|---|---|---|---|---|---|---|---|---|
| | | | Day | Day | Day | Day | Day | Day | Day | Day | Day | Day | Day | Day | Day | Day | Day | Day |
| Group | Treatment | ID# | −1 | 0 | 1 | 2 | 3 | 4 | 5 | 6 | 7 | 8 | 9 | 10 | 11 | 12 | 13 | 14 |
| 1 | Sham | 1 | 302 | 309 | 312 | 315 | 324 | 333 | 334 | 337 | 345 | 347 | 350 | 360 | 363 | 363 | 374 | 379 |
| | | 2 | 285 | 291 | 287 | 289 | 291 | 301 | 308 | 310 | 318 | 321 | 325 | 332 | 334 | 342 | 343 | 352 |
| | | 3 | 291 | 299 | 294 | 294 | 278 | 284 | 290 | 295 | 301 | 302 | 311 | 315 | 318 | 319 | 324 | 331 |
| | | 4 | 285 | 301 | 297 | 301 | 309 | 319 | 332 | 339 | 352 | 362 | 374 | 378 | 386 | 393 | 400 | 410 |
| | | 5 | 298 | 315 | 302 | 307 | 315 | 325 | 334 | 305 | 313 | 320 | 335 | 339 | 343 | 353 | 356 | 363 |
| | | 6 | 291 | 295 | 295 | 297 | 305 | 317 | 322 | 325 | 335 | 336 | 340 | 343 | 350 | 355 | 369 | 374 |
| | | 7 | 302 | 309 | 308 | 314 | 328 | 324 | 333 | 343 | 352 | 369 | 362 | 370 | 377 | 380 | 388 | 395 |
| | | 8 | 297 | 302 | 302 | 305 | 302 | 309 | 318 | 321 | 326 | 329 | 334 | 341 | 345 | 344 | 348 | 355 |
| 2 | UUO + | 1 | 299 | 310 | 293 | 290 | 294 | 297 | 305 | 310 | 296 | 310 | 324 | 324 | 330 | 336 | 346 | 347 |
| | Vehicle | 2 | 288 | 289 | 296 | 291 | 293 | 302 | 306 | 316 | 325 | 332 | 338 | 342 | 354 | 355 | 359 | 370 |
| | | 3 | 283 | 284 | 269 | 268 | 263 | 263 | 266 | 257 | 251 | 258 | 264 | 272 | 272 | 285 | 295 | 296 |
| | | 4 | 294 | 304 | 293 | 289 | 297 | 300 | 308 | 307 | 317 | 322 | 327 | 340 | 339 | 336 | 347 | 351 |
| | | 5 | 301 | 308 | 279 | 274 | 272 | 281 | 280 | 287 | 292 | 290 | 295 | 295 | 299 | 307 | 316 | 321 |
| | | 6 | 297 | 292 | 280 | 276 | 284 | 293 | 297 | 297 | 304 | 312 | 315 | 316 | 326 | 320 | 330 | 332 |
| | | 7 | 304 | 317 | 295 | 285 | 294 | 294 | 305 | 307 | 321 | 327 | 336 | 347 | 351 | 360 | 364 | 377 |
| | | 8 | 285 | 290 | 285 | 284 | 289 | 296 | 286 | 297 | 307 | 297 | 305 | 309 | 317 | 322 | 330 | 341 |
| 3 | UUO + | 1 | 284 | 304 | 290 | 283 | 286 | 285 | 283 | 283 | 290 | 295 | 305 | 300 | 298 | 295 | 294 | 298 |
| | Compound | 2 | 304 | 308 | 296 | 294 | 292 | 288 | 290 | 290 | 285 | 284 | 284 | 284 | 297 | 291 | 293 | 296 |
| | A | 3 | 299 | 304 | 304 | 297 | 298 | 301 | 300 | 297 | 303 | 304 | 310 | 311 | 311 | 319 | 318 | 322 |
| | (10 mg/kg) | 4 | 289 | 291 | 281 | 281 | 280 | 277 | 278 | 281 | 280 | 279 | 279 | 277 | 278 | 283 | 286 | 291 |
| | @QD | 5 | 297 | 306 | 279 | 275 | 276 | 275 | 268 | 266 | 260 | 262 | 284 | 270 | 271 | 271 | 270 | 281 |
| | | 6 | 301 | 307 | 298 | 290 | 288 | 294 | 285 | 290 | 299 | 303 | 304 | 304 | 315 | 315 | 320 | 331 |
| | | 7 | 291 | 300 | 285 | 280 | 276 | 277 | 272 | 275 | 274 | 275 | 275 | 279 | 274 | 277 | 282 | 280 |
| | | 8 | 286 | 295 | 271 | 277 | 273 | 270 | 273 | 274 | 278 | 278 | 285 | 289 | 294 | 303 | 301 | 302 |

# Each animal actually had a different number, and the animal numbers are shown for simplicity as 1-8 for each group.

TABLE 25B
Mean + SD of Body Weight of Rats in UUO Study

| | | Body Weight/g | | | | | | | |
|---|---|---|---|---|---|---|---|---|---|
| | | Day | Day | Day | Day | Day | Day | Day | Day |
| Group | Treatment | −1 | 0 | 1 | 2 | 3 | 4 | 5 | 6 |
| 1 | Sham | 294 ± 7 | 303 ± 8 | 300 ± 8 | 303 ± 9 | 307 ± 17 | 314 ± 16 | 321 ± 16 | 322 ± 17 |
| 2 | UUO + | 294 ± 8 | 299 ± 12 | 286 ± 10 | 282 ± 8 | 286 ± 12 | 291 ± 13 | 294 ± 15 | 297 ± 19 |
| | Vehicle | | | | | | | | |
| 3 | UUO + | 294 ± 7 | 302 ± 6 | 288 ± 11 | 285 ± 8 | 284 ± 9 | 283 ± 11 | 281 ± 11 | 282 ± 10 |
| | Compound | | | | | | | | |
| | A | | | | | | | | |
| | | Body Weight/g | | | | | | | |
| | | Day | Day | Day | Day | Day | Day | Day | Day |
| | Group | 7 | 8 | 9 | 10 | 11 | 12 | 13 | 14 |
| | 1 | 330 ± 19 | 336 ± 23 | 341 ± 20 | 347 ± 21 | 352 ± 22 | 356 ± 23 | 363 ± 25 | 370 ± 25 |
| | 2 | 302 ± 24 | 306 ± 24 | 313 ± 25 | 318 ± 26 | 324 ± 27 | 328 ± 25 | 336 ± 23 | 342 ± 26 |
| | 3 | 284 ± 14 | 285 ± 15 | 291 ± 13 | 289 ± 14 | 292 ± 17 | 294 ± 17 | 296 ± 17 | 300 ± 18 |

TABLE 26
Clinical Observation/Assessment of Rats in UUO Study

| | | | Clinical Observation | | | | | | | |
|---|---|---|---|---|---|---|---|---|---|---|
| | | No. | Day | Day | Day | Day | Day | Day | Day | Day |
| Group | Treatment | # | 0 | 1 | 2 | 3 | 4 | 5 | 6 | 7 |
| 1 | Sham | 1 | — | — | — | — | — | — | — | — |
| | | 2 | — | — | — | — | — | — | — | — |
| | | 3 | — | — | — | — | — | — | — | — |
| | | 4 | — | — | — | — | — | soft | — | — |
| | | | | | | | | stool | | |
| | | 5 | — | — | — | — | — | — | — | — |
| | | 6 | — | — | — | — | — | — | — | — |
| | | 7 | — | — | — | — | — | soft | — | — |
| | | | | | | | | stool | | |
| | | 8 | — | — | — | — | — | — | — | — |
| 2 | UUO + | 1 | — | — | — | — | — | — | — | — |
| | Vehicle | 2 | — | — | — | — | — | — | — | — |
| | | 3 | — | — | — | — | — | — | — | — |
| | | 4 | — | — | — | — | — | — | — | — |
| | | 5 | — | — | — | — | — | — | — | — |
| | | 6 | — | — | — | — | — | — | — | — |
| | | 7 | — | — | — | — | — | — | — | — |
| | | 8 | — | — | — | — | — | — | — | — |
| 3 | UUO + | 1 | — | — | — | — | — | — | — | — |
| | Compound | 2 | — | — | — | — | — | — | — | — |
| | A | 3 | — | — | — | — | — | — | — | — |
| | | 4 | — | — | — | — | — | — | — | — |
| | | 5 | — | — | — | — | — | — | — | — |
| | | 6 | — | — | — | soft | — | — | — | — |
| | | | | | | stool | | | | |
| | | 7 | — | — | — | — | — | — | — | — |
| | | 8 | — | — | — | — | — | — | — | — |
| | | | | Clinical Observation | | | | | | |
| | | | No. | Day | Day | Day | Day | Day | Day | Day |
| | Group | Treatment | # | 8 | 9 | 10 | 11 | 12 | 13 | 14 |
| | 1 | Sham | 1 | — | — | — | — | — | — | — |
| | | | 2 | — | — | — | — | — | — | — |
| | | | 3 | — | — | — | — | — | — | — |
| | | | 4 | — | — | — | — | — | — | — |
| | | | 5 | — | — | — | — | — | — | — |
| | | | 6 | — | — | — | — | — | — | — |
| | | | 7 | — | — | — | — | — | — | — |
| | | | 8 | — | — | — | — | — | — | — |
| | 2 | UUO + | 1 | — | — | — | — | — | — | — |
| | | Vehicle | 2 | — | — | — | — | — | — | — |
| | | | 3 | — | — | — | — | — | — | — |
| | | | 4 | — | — | — | — | — | — | — |
| | | | 5 | — | — | — | — | — | — | — |
| | | | 6 | — | — | — | — | — | — | — |
| | | | 7 | — | — | — | — | — | — | — |
| | | | 8 | — | — | — | — | — | — | — |
| | 3 | UUO + | 1 | — | — | — | — | — | — | — |
| | | Compound | 2 | — | — | — | — | — | — | — |
| | | A | 3 | — | — | — | — | — | — | — |
| | | | 4 | — | — | — | — | — | — | — |
| | | | 5 | — | — | — | — | — | — | — |
| | | | 6 | — | — | — | — | — | — | — |
| | | | 7 | — | — | — | — | — | — | — |
| | | | 8 | — | — | — | — | — | — | — |

# Each animal actually had a different number, and the animal numbers are shown for simplicity as 1-8 for each group.

TABLE 27
Clinical Scores of Glomerulosclerosis, Interstitial Nephritis, Collagen
Fiber Deposition, Nephropathy, as well as Total Scores in Rats in UUO Study

| | Mouse | | Interstitial | Collagen fiber | | |
|---|---|---|---|---|---|---|
| Group | No. | Glomerulosclerosis | nephritis | deposition | Nephropathy | Total score |
| Sham | 1 | 0 | 0 | 0 | 0 | 0 |
| | 2 | 0 | 0 | 0 | 0 | 0 |
| | 3 | 0 | 0 | 0 | 0 | 0 |
| | 4 | 0 | 0 | 0 | 0 | 0 |
| | 5 | 0 | 0 | 0 | 0 | 0 |
| | 6 | 0 | 0 | 0 | 0 | 0 |
| | 7 | 0 | 0 | 0 | 0 | 0 |
| | 8 | 0 | 0 | 0 | 0 | 0 |
| | 1 | 0 | 4 | 3 | 2 | 9 |
| UUO + | 2 | 0 | 4 | 2 | 3 | 9 |
| Vehicle | 3 | 0 | 4 | 3 | 2 | 9 |
| | 4 | 0 | 4 | 3 | 2 | 9 |
| | 5 | 0 | 4 | 2 | 3 | 9 |
| | 6 | 0 | 4 | 3 | 2 | 9 |
| | 7 | 0 | 3 | 3 | 2 | 8 |
| | 8 | 0 | 4 | 3 | 3 | 10 |
| UUO + | 1 | 0 | 2 | 1 | 1 | 4 |
| Compound | 2 | 0 | 3 | 2 | 2 | 7 |
| A | 3 | 0 | 2 | 1 | 2 | 5 |
| | 4 | 0 | 2 | 1 | 2 | 5 |
| | 5 | 0 | 3 | 3 | 2 | 8 |
| | 6 | 0 | 3 | 2 | 2 | 7 |
| | 7 | 0 | 2 | 1 | 2 | 5 |
| | 8 | 0 | 3 | 2 | 2 | 7 |

# Each animal actually had a different number, and the animal numbers are shown for simplicity as 1-8 for each group.

TABLE 28
UUO + Compound A Pharmacokinetic Profile in Male SD Rats

| | Concentration (ng/ml) | | | | | | | | | | |
|---|---|---|---|---|---|---|---|---|---|---|---|
| | Test | Test | Test | Test | Test | Test | Test | Test | | | |
| Time | Subject | Subject | Subject | Subject | Subject | Subject | Subject | Subject | Mean | SD | CV |
| (h) | 1 | 2 | 3 | 4 | 5 | 6 | 7 | 8 | (ng/ml) | (ng/ml) | (%) |
| 4 | 220 | 318 | 133 | 215 | 132 | 194 | 198 | 195 | 201 | 58 | 29.0 |

TABLE 29A
RNA levels of Tissue Biomarkers in Rats of UUO Study

| | | | Normalized Fold Change | | | | | | | |
|---|---|---|---|---|---|---|---|---|---|---|
| Group | Treatment | ID# | Col1a1 | TGF-b1 | MCP-1 | IL-1b | IL-6 | IL-17 | TNF-a | Timp1 |
| 1 | Sham | 1 | 0.01 | 0.07 | 0.09 | 0.55 | 0.05 | ND | 0.30 | 0.00 |
| | | 2 | 0.01 | 0.04 | 0.04 | 0.25 | ND | ND | 0.19 | 0.00 |
| | | 3 | 0.00 | 0.02 | 0.03 | 0.22 | ND | ND | 0.07 | 0.00 |
| | | 4 | 0.01 | 0.08 | 0.08 | 0.47 | 0.03 | ND | 0.27 | 0.01 |
| | | 5 | 0.00 | 0.03 | 0.05 | 0.36 | 0.02 | ND | 0.10 | 0.00 |
| | | 6 | 0.00 | 0.03 | 0.05 | 0.18 | ND | ND | 0.14 | 0.00 |
| | | 7 | 0.00 | 0.02 | 0.05 | 0.14 | ND | ND | 0.07 | 0.00 |
| | | 8 | 0.00 | 0.01 | 0.03 | 0.12 | 0.01 | ND | 0.05 | 0.00 |
| 2 | UUO + Vehicle | 1 | 0.82 | 0.89 | 0.82 | 0.97 | 0.81 | 0.96 | 0.87 | 0.86 |
| | | 2 | 0.85 | 1.33 | 1.15 | 1.00 | 0.58 | 1.82 | 1.27 | 1.10 |
| | | 3 | 1.06 | 0.86 | 1.39 | 1.01 | 1.33 | 1.77 | 1.10 | 0.77 |
| | | 4 | 1.59 | 1.20 | 1.30 | 1.82 | 2.12 | 1.34 | 1.35 | 1.76 |
| | | 5 | 0.41 | 0.57 | 0.70 | 0.56 | 0.45 | 0.60 | 0.67 | 0.58 |
| | | 6 | 1.22 | 1.09 | 0.73 | 0.71 | 0.97 | 0.58 | 0.78 | 0.92 |
| | | 7 | 1.32 | 1.36 | 1.28 | 1.19 | 0.96 | 0.53 | 1.14 | 1.21 |
| | | 8 | 0.73 | 0.70 | 0.62 | 0.74 | 0.78 | 0.39 | 0.81 | 0.81 |
| 3 | UUO + | 1 | 0.51 | 0.71 | 0.48 | 0.37 | 0.11 | 0.37 | 1.50 | 0.83 |
| | Compound A | 2 | 0.31 | 0.42 | 0.24 | 0.28 | 0.09 | 0.14 | 1.16 | 0.41 |
| | (10mg/kg) | 3 | 0.19 | 0.28 | 0.18 | 0.24 | 0.04 | 0.26 | 1.00 | 0.36 |
| | @QD | 4 | 0.50 | 0.58 | 0.34 | 0.24 | 0.14 | 0.51 | 0.79 | 0.72 |
| | | 5 | 0.50 | 0.64 | 0.44 | 0.45 | 0.18 | 0.12 | 1.05 | 0.66 |
| | | 6 | 0.40 | 0.40 | 0.25 | 0.23 | 0.11 | 0.45 | 0.62 | 0.09 |
| | | 7 | 0.34 | 0.64 | 0.50 | 0.40 | 0.13 | 0.72 | 1.20 | 0.72 |
| | | 8 | 0.41 | 0.50 | 0.20 | 0.24 | 0.05 | 0.20 | 0.64 | 0.52 |

# Each animal actually had a different number, the animal numbers are shown for simplicity as 1-8 for each group.

TABLE 29B
Mean + SEM RNA levels of Tissue Biomarkers in Rats of UUO Study

| | | | Mean ± SEM | | | | | | | |
|---|---|---|---|---|---|---|---|---|---|---|
| Group | Treatment | N | Col1a1 | TGF-b1 | MCP-1 | IL-1b | IL-6 | IL-17 | TNF-a | Timp1 |
| 1 | Sham | 8 | 0.01 ± 0.00 | 0.04 ± 0.01 | 0.05 ± 0.01 | 0.29 ± 0.06 | 0.03 ± 0.01 | ND | 0.15 ± 0.03 | 0.00 ± 0.00 |
| 2 | UUO + | 8 | 1.00 ± 0.13 | 1.00 ± 0.10 | 1.00 ± 0.11 | 1.00 ± 0.14 | 1.00 ± 0.19 | 1.00 ± 0.20 | 1.00 ± 0.09 | 1.00 ± 0.13 |
| | Vehicle | | | | | | | | | |
| 3 | UUO + | 8 | 0.39 ± 0.04 | 0.52 ± 0.05 | 0.33 ± 0.05 | 0.31 ± 0.03 | 0.11 ± 0.02 | 0.35 ± 0.07 | 1.00 ± 0.11 | 0.54 ± 0.09 |
| | Compound | | | | | | | | | |
| | A | | | | | | | | | |

TABLE 30A
Serum Urea levels of Rats in UUO Study

| | | | Serum Urea |
|---|---|---|---|
| Group | Treatment | ID# | (mmol/L) |
| 1 | Sham | 1 | 4.12 |
| | | 2 | 4.57 |
| | | 3 | 5.38 |
| | | 4 | 4.68 |
| | | 5 | 4.96 |
| | | 6 | 5.76 |
| | | 7 | 5.30 |
| | | 8 | 6.23 |
| 2 | UUO + | 1 | 6.27 |
| | Vehicle | 2 | 4.62 |
| | | 3 | 7.03 |
| | | 4 | 5.44 |
| | | 5 | 6.21 |
| | | 6 | 6.63 |
| | | 7 | 5.93 |
| | | 8 | 6.14 |
| 3 | UUO + | 1 | 5.72 |
| | Compound | 2 | 5.03 |
| | A | 3 | 5.64 |
| | (10 mg/kg)@ | 4 | 6.28 |
| | QD | 5 | 5.22 |
| | | 6 | 4.77 |
| | | 7 | 6.97 |
| | | 8 | 5.71 |

TABLE 30B
Mean + S.D. Serum Urea levels of Rats in UUO Study

| Group | Treatment | Serum Urea (mmol/L) |
|---|---|---|
| 1 | Sham | 5.13 ± 0.68 |
| 2 | UUO + Vehicle | 6.03 ± 0.74 |
| 3 | VVU + Compound A (10 | 5.67 ± 0.71 |
| | mg/kg) @ QD | |

# Each animal in Table 29A actually had a different number, and the animal numbers are shown for simplicity as 1-8 for each group.

TABLE 31
Hydroxyproline concentration of Rats in UUO Study

| | | | Final | | | |
|---|---|---|---|---|---|---|
| | | | Hydroxyproline | | | |
| | | | Con. | | | |
| Group | Treatment | ID | (μg/mg tissue) | Mean | SEM | Mean ± SEM |
| 1 | Sham | 1 | 0.56 | 0.70 | 0.04 | 0.70 ± 0.04 |
| | | 2 | 0.84 | | | |
| | | 3 | 0.65 | | | |
| | | 4 | 0.55 | | | |
| | | 5 | 0.83 | | | |
| | | 6 | 0.75 | | | |
| | | 7 | 0.63 | | | |
| | | 8 | 0.81 | | | |
| 2 | UUO + | 1 | 0.58 | 0.69 | 0.03 | 0.69 ± 0.03 |
| | Vehicle | 2 | 0.80 | | | |
| | | 3 | 0.65 | | | |
| | | 4 | 0.63 | | | |
| | | 5 | 0.80 | | | |
| | | 6 | 0.67 | | | |
| | | 7 | 0.75 | | | |
| | | 8 | 0.67 | | | |
| 3 | UUO + | 1 | 0.69 | 0.66 | 0.05 | 0.66 ± 0.05 |
| | Compound | 2 | 0.86 | | | |
| | A | 3 | 0.63 | | | |
| | | 4 | 0.61 | | | |
| | | 5 | 0.61 | | | |
| | | 6 | 0.64 | | | |
| | | 7 | 0.44 | | | |
| | | 8 | 0.80 | | | |

# Each animal actually had a different number from 1 to 88, and the animal numbers are shown for simplicity as 1-8 for each group.

### Example 121: Idiopathic Pulmonary Fibrosis in Animals Treated with a BET Inhibitor and Vehicle

The idiopathic pulmonary fibrosis (1FF) model was used to determine the impact of Compound A in a model of PF. IPF is induced in mice by a single intratracheal dose of bleomycin (Bleo).

### Experimental Method F: IFF in Animals Treated with a BET Inhibitor and Vehicle

Male C57Bl/6J mice, aged 12 weeks with an approximate weight of 25-30 g were obtained from Jackson Laboratories (Bar Harbor, ME). Animals were acclimated for two weeks prior to study initiation, and were randomized into the experimental groups outlined in Table 32.

TABLE 32
Experimental Groups

| | | | | | | Route, |
|---|---|---|---|---|---|---|
| | | | | Bleo/Control | | Freq, |
| Group | Treatment | Sample Size | Bleo | Route, Day | Agent | Start |
| 1 | Saline | 10 per group | PBS | IT, Day 0 | Saline | OG, QOD, |
| | control | (experiments | | | | D7 |
| | | began with | | | | |
| 2 | Bleo control | approximately | 1 U/kg | IT, Day 0 | Saline | OG, QOD, |
| | | 14-16 mice per | | | | D7 |
| | | group, and only | | | | |
| 3 | Bleo + Compound | mice with | 1 U/kg | IT, Day 0 | Compound A | OG, QOD, |
| | A (1 mg/kg) | confirmed | | | (1 mg/kg) | D7 |
| | | fibrosis | | | | |
| 4 | Bleo + Compound | (n = 10) | 1 U/kg | IT, Day 0 | Compound A | OG, QOD, |
| | A (3 mg/kg) | proceeded | | | (3 mg/kg) | D7 |
| | | past day 7) | | | | |
| 5 | Bleo + Compound | | 1 U/kg | IT, Day 0 | Compound A | OG, QOD, |
| | A (10 mg/kg) | | | | (10 mg/kg) | D7 |
| 6 | Bleo + Pirfenidone | | 1 U/kg | IT, Day 0 | Pirfenidone | OG, QOD, |
| | (300 mg/kg) | | | | (300 mg/kg) | D7 |

Intratracheal (IT) administration of Bleo or PBS control was conducted at the start of the study (Day 0). The IT route ensured precise delivery of material into the superior pulmonary apparatus, with subsequent access to the bronchial tree and parenchyma. Animals were weighed on Day 0, and indexed boluses of PBS control (Group 1) or Bleo (Groups 2-6) were delivered by IT administration.

Following administration of Bleo, animals (n=10/group) were included in the study if they met the following guidelines on Day 7: (1) a predictable 5% loss in body weight, and (2) the presence of fibrotic pulmonary lesions, detected by micro-CT imaging. 10 animals from Group 1 additionally proceeded as controls. Animals that continued in the study on Day 7 received an oral gavage (OG) administration of PBS, Compound A (1 mg/kg, 3 mg/kg, 10 mg/kg), or Pirfenidone (300 mg/kg), as outlined in Table 31 in 25 μL boluses every other day (QOD), starting on Day 7. Compound A was formulated in 5% (v/v) DMSO/15% (v/v) PEG 400/80% (v/v) E-TPGS in purified water.

Body weights were obtained at Day 0 at the start of the study, and again at Day 7 to determine if an animal continued in the study. Body weight was measured daily from Day 7 until Day 21, and oxygen saturation was measured by a pulse oximetry (MouseOx) system QOD from Day 7 until study completion on Day 21, and results were plotted and analyzed by Kaplan-Meier plots.

Micro-CT imaging was also performed on Day 7 to determine relative fibrosis and on Day 21. Micro-CT imaging of animal lungs was performed using a Quantum GX Micro-CT (Perkin Elmer, Inc.) comprising a tungsten anode following anesthesia induction and maintenance using 2% isoflurane. Images were acquired with a respiratory gated technique with the following parameters. X-ray tube voltage: 90 KV; X-ray tube current: 88 pA; total scan time: 4 minutes. The system was calibrated monthly with standard phantoms for noise, uniformity, low contrast, and resolution to ensure consistency.

Animals were euthanized on Day 21 following Micro-CT imaging, and the right lung was collected for biochemical analysis, and the left lung was inflation-fixed for morphological assessments. A resection of the right lung was weighed and homogenized to determine total hydroxyproline levels by colorimetric assay, and the remaining lung lysate was saved for future analyses.

For the morphological assessments, the left lung was inflation-fixed at 25 cm of water pressure with 4% paraformaldehyde in PBS for 1 minute, processed, and then sectioned. Morphological analysis of the lung was conducted using H&E-stained slides. Additional histologic stains for morphological analyses included Masson's Trichrome staining with Ashcroft scoring to determine severity of fibrosis.

### Efficacy of Oropharyngeal Administration of Compound A in the IPF Mouse Model

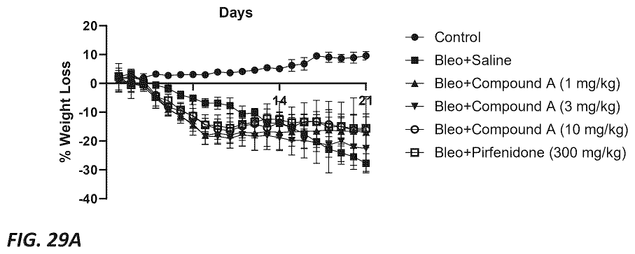

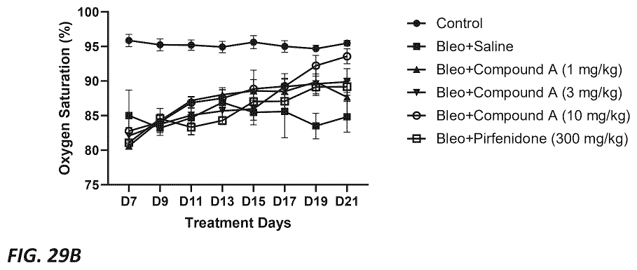

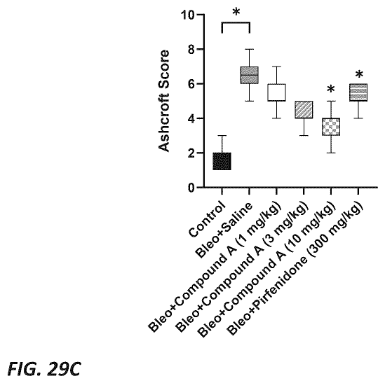

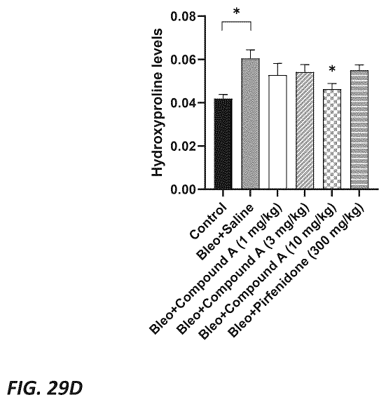

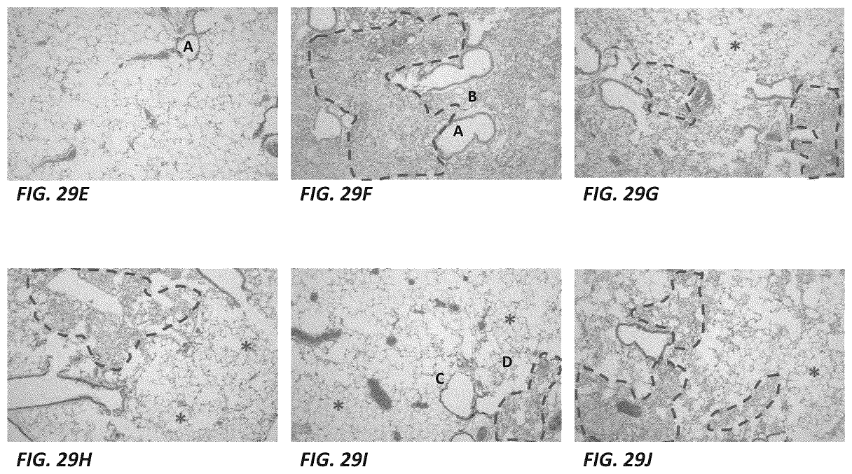

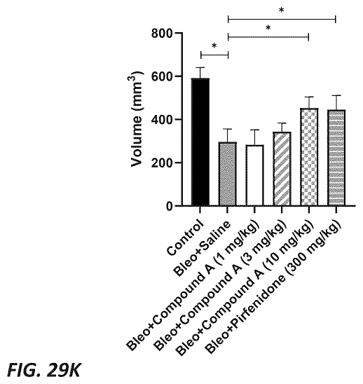

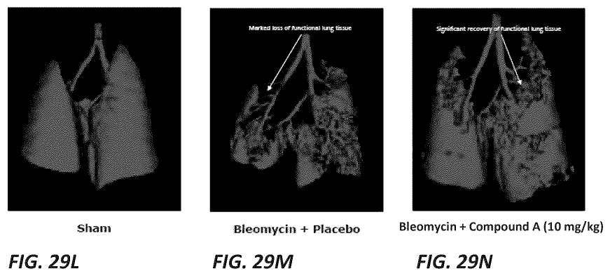

As expected, FIG. 29A shows that animals in Group 1 (PBS control) exhibited no weight loss over the first 7 days, and steadily gained weight over the course of the study because IPF was not induced. Animals in Groups 2-6 all lost weight over the first 7 days following Bleo administration and prior to administration of the compounds, and exhibited significant decreases in body weight relative to animals in Group 1. Animals receiving the vehicle control (Group 2) exhibited a steady and constant weight loss over the 21 Day study period. Weight loss continued after administration of Compound A (all doses tested) began until approximately Day 8, and then weight loss was arrested, and animal weights remained at a relatively constantly level through the remainder of the study. Animals receiving the highest dose of Compound A (10 mg/kg) exhibited a higher ending weight at Day 21 than animals receiving the two lower doses of Compound A (1 mg/kg and 3 mg/kg). The high dose of Compound A (10 mg/kg) additionally resulted in a weight loss trajectory that was similar to animals receiving pirfenidone (300 mg/kg). No significant difference was measured when comparing Bleo+Saline treated animals with animals receiving Compound A (all doses tested) and Pirfenidone (300 mg/kg). Table 33A additionally provides the mean body weight change as depicted in FIG. 29A.

FIG. 29B illustrates changes in oxygen saturation, measured QOD from Day 7 until Day 21. While animals in Group 1 (control group where IPF was not induced) maintained constant oxygen saturation levels of approximately 95% over the course of the study, animals in Groups 2-6 demonstrated decreased oxygen saturation levels at Day 7 relative to oxygen saturation levels for animals in Group 1. Animals in the IPF control group (Group 2; Bleo+Saline) maintained decreased oxygen saturation levels over the course of the study, with oxygen saturation levels of about 85% at Day 21. Animals receiving Compound A (3 mg/kg, and 10 mg/kg) demonstrated a significant recovery in oxygen saturation levels relative to animals in the IPF control group (Group 2; Bleo+Saline). Animals receiving 10 mg/kg of Compound A had oxygen saturation levels of about 93.6% at Day 21, nearly restored to the level in Group 1 (control group with no IPF; about 95.5%), and animals receiving 3 mg/kg of Compound A had oxygen saturation levels of about 90% by Day 21, demonstrating a recovery in oxygen saturation following administration of Compound A. Animals receiving the low dose of Compound A (1 mg/kg) additionally demonstrated a recovery in oxygen saturation, albeit at levels lower than animals receiving the two higher doses of Compound A (3 mg/kg and 10 mg/kg), and oxygen saturation for animals in this group (Group 3) was similar to that of animals receiving Pirfenidone (Group 6). Table 33B additionally provides the mean oxygen saturation as depicted in FIG. 29B.

FIG. 29C and Table 33C depict Ashcroft scoring of fibrosis at Day 21 for animals in the study. Ashcroft scoring is made on a scale of 0-8, with 8 being the most severe. Animals in Group 1 (control group with no IPF induction) demonstrated low Ashcroft scoring, whereas animals in Group 2 (IPF control group) exhibited elevated Ashcroft scoring at Day 21 that was significantly increased relative to Ashcroft scoring from Group 1. Compound A decreased Ashcroft scores relative to animals in Group 2 (IPF control group) in a dose-dependent manner, with animals receiving the highest dose of Compound A (10 mg/kg) exhibiting the most significant reduction in Ashcroft scores. Animals in this group (Group 5; Compound A 10 mg/kg) demonstrated significantly reduced Ashcroft scores, indicating reduced fibrosis, relative to animals in the IPF control group (Group 2; Bleo+Saline). Animals receiving Pirfenidone (Group 6; 300 mg/kg) additionally exhibited significantly reduced Ashcroft scores relative to the IPF control group (Group 2; Bleo+Saline), but scores for Group 6 although similar to the scores for Group 3 were not as low as those in Groups 4 and 5.

FIG. 29D and Table 33D depict hydroxyproline content in lung lysates, collected at Day 21 following necropsy. Hydroxyproline is an indirect indicator of collagen content and is a biomarker for IPF. Animals in the IPF control group (Group 2; Bleo+Saline) exhibited significantly elevated levels of hydroxyproline relative to animals in Group 1 (control group where IPF was not induced). Animals in Groups 3 and 4 (Compound A 1 mg/kg and 3 mg/kg, respectively) demonstrated reduced levels of hydroxyproline relative to Group 2, and levels of hydroxyproline for animals in Groups 3 and 4 were similar to hydroxyproline levels from animals in Group 6 (Bleo+Pirfenidone 300 mg/kg). Animals receiving the high dose of Compound A (10 mg/kg) exhibited significantly reduced levels of hydroxyproline that were about 60.4% lower than hydroxyproline levels in the IPF control group (Group 2; Bleo+Saline), and hydroxyproline levels were nearly restored to levels measured in the control group (Group 1 where IPF was not induced).

Histopathological analysis of lung tissue (FIG. 29E-29J) was performed using Masson's Trichrome as described above. Fibrotic phenotype was observed in lung samples from Group 2 (Bleo+Saline) mice compared to Controls (vehicle only). In contrast, fibrosis was observed to be qualitatively diminished in Group 5 (Bleo+Compound A (10 mg/mL)) mice and Group 6 (Bleo+Pirfenidone) mice compared to Group 2 (Bleo+Saline) mice. Animals in Group 1 (no IPF; FIG. 29E) demonstrated normal parenchyma with no fibrotic lesions and conducting airways (labeled with “A”). Animals in Group 2 (IPF control (Bleo+Saline); FIG. 29F) demonstrated few normal respiratory zones, with areas of intense fibrosis (hashed outline). Lung tissue from these animals also demonstrated conducting airways (“A”) and sporadic airways (labeled with “B”). Animals in Group 3 (Bleo+Compound A 1 mg/kg; FIG. 29G) demonstrated some normal respiratory zones (marked by *), as well as areas of fibrosis (hashed outline). These areas of fibrosis were generally smaller and less intense than the fibrosis observed in Group 2. Animals in Group 4 (Bleo+Compound A 3 mg/kg; FIG. 29H) demonstrated normal respiratory zones (marked by *) and areas of intense fibrosis (hashed outline). Animals in Group 5 (Bleo+Compound A 10 mg/kg; FIG. 29I) demonstrated larger normal respiratory zones (marked by *) and smaller areas of intense fibrosis (hashed outline). Tissue samples from these animals further demonstrated less severe localized fibrosis at airways (labeled with “C”) and less severe localized fibrosis in the parenchyma (labeled with “D”). Animals in Group 6 (Bleo+Pirfenidone 300 mg/kg; FIG. 29J) also demonstrated normal respiratory zones (marked by *) and areas of intense fibrosis (hashed outline). Without being bound by any particular theory, Compound A appeared to increase the relative proportion of normal respiratory zones in the samples in a dose-dependent manner, with animals in Group 5 (Compound A 10 mg/kg) demonstrating large normal respiratory zones with small areas of fibrosis.

FIG. 29K shows mean functional lung volume in animals, as measured on Day 21. Animals in Group 2 (IPF control group) demonstrated significantly reduced (p<0.05) lung volume relative to Group 1 (control group where IPF was not induced). Compound A protected against lung volume reduction in a dose-dependent manner, with animals in Group 5 (Compound A 10 mg/kg) demonstrating a significantly increased (p<0.05) lung volume relative to animals in Group 2 (IPF control group). Animals in Group 5 demonstrated about a 52.7% mean improvement in functional lung volume compared to animals in Group 2. Lung volume for animals in Group 6 was similar to lung volume from animals in Group 5, and was also significantly increased (p<0.05) relative to lung volume from animals in Group 2 (IPF control group). FIGS. 29L-29M depict representative CT images of animals from Group 1 (Saline+Saline; FIG. 29L), Group 2 (Bleo+Saline; FIG. 29M), and Group 5 (Bleo+Compound A 10 mg/kg; FIG. 29N). As noted above, Compound A proved effective in improving functional lung volume relative to animals in Group 2, and improved/restored lung volume in a dose-dependent manner. The increase in functional lung volume correlated with increased blood oxygen saturation and reduced Ashcroft scoring. Table 33E additionally provides the mean functional lung volume as depicted in FIG. 29K.

OG administration of Compound A also was observed to be safe, as no deaths or significant changes in mortality were observed throughout the study. Without being bound by any particular theory, Compound A proved effective in protecting against body weight loss associated with IPF. Compound A additionally proved effective in arresting/slowing fibrosis, restoring lung function and protecting against fibrosis, demonstrated by increased oxygen saturation levels relative to placebo control, decreased Ashcroft scoring for lung fibrosis relative to placebo control, diminished levels of hydroxyproline relative to placebo control, increased functional lung volume relative to placebo control, and improved lung morphology relative to placebo control. Thus, Compound A appears to ameliorate Bleo-induced fibrosis at least as well as pirfenidone is able to arrest or slow fibrosis and protects against adverse histopathology associated with IPF. Without being bound by any theory given the dose-dependent efficacy with the highest dose being observed to have the greatest effect it may be that by increasing doses higher than 10 mg/kg further improvements may be achieved.

TABLE 33A
Mean Body Weight Change.

| | | | | | Bleo + Compound A | | Bleo + Compound A | | Bleo + Compound A | | Bleo + Pirfenidone | |
|---|---|---|---|---|---|---|---|---|---|---|---|---|
| | Vehicle | | Bleo + Saline | | (1 mg/kg) | | (3 mg/kg) | | (10 mg/kg) | | (300 mg/kg) | |
| Day | Mean | SD | Mean | SD | Mean | SD | Mean | SD | Mean | SD | Mean | SD |
| 1 | 1.462 | 0.314 | 2.214 | 0.617 | 1.269 | 3.990 | 0.712 | 3.764 | 1.987 | 1.795 | 2.308 | 4.545 |
| 2 | 1.888 | 0.345 | 3.410 | 0.990 | −0.061 | 3.075 | −0.088 | 2.711 | 2.172 | 2.734 | −0.484 | 4.757 |
| 3 | 1.986 | 0.497 | 0.573 | 0.303 | −0.538 | 1.681 | −1.018 | 1.742 | 0.974 | 2.422 | −0.051 | 2.047 |
| 4 | 3.229 | 0.723 | −0.550 | 0.297 | −4.382 | 1.043 | −5.198 | 3.450 | −4.101 | 3.786 | −4.604 | 4.003 |
| 5 | 2.742 | 0.558 | −1.734 | 1.455 | −8.986 | 1.806 | −8.915 | 2.241 | −8.131 | 3.261 | −5.992 | 2.195 |
| 6 | 3.045 | 0.339 | −4.037 | 1.363 | −11.204 | 1.781 | −11.426 | 2.411 | −9.560 | 2.943 | −9.221 | 2.211 |
| 7 | 3.067 | 0.611 | −5.101 | 1.038 | −14.465 | 2.175 | −14.024 | 3.964 | −11.173 | 3.875 | −11.372 | 2.717 |
| 8 | 2.960 | 0.482 | −6.828 | 1.559 | −18.012 | 3.042 | −18.310 | 2.893 | −14.286 | 3.400 | −14.446 | 2.345 |
| 9 | 3.908 | 0.366 | −6.884 | 2.123 | −17.310 | 3.401 | −18.416 | 2.703 | −15.876 | 3.478 | −14.694 | 3.760 |
| 10 | 3.669 | 0.400 | −7.744 | 2.723 | −17.947 | 4.574 | −19.029 | 3.415 | −16.428 | 4.429 | −15.574 | 4.909 |
| 11 | 4.139 | 0.584 | −10.572 | 2.726 | −16.922 | 4.804 | −17.826 | 4.014 | −14.809 | 4.963 | −14.209 | 4.731 |
| 12 | 4.590 | 0.841 | −10.275 | 1.592 | −17.204 | 6.018 | −18.384 | 4.880 | −13.953 | 4.983 | −13.437 | 4.611 |
| 13 | 5.449 | 0.954 | −13.746 | 1.291 | −16.710 | 6.220 | −18.055 | 4.860 | −15.394 | 6.495 | −12.258 | 5.162 |
| 14 | 5.090 | 1.150 | −13.934 | 4.027 | −16.996 | 6.009 | −18.840 | 4.497 | −13.610 | 3.505 | −12.644 | 6.430 |
| 15 | 6.236 | 2.075 | −15.362 | 4.588 | −16.840 | 4.949 | −19.765 | 5.038 | −15.280 | 4.393 | −13.627 | 5.427 |
| 16 | 6.764 | 2.137 | −17.518 | 5.095 | −16.698 | 5.811 | −19.797 | 6.103 | −13.218 | 2.599 | −13.391 | 6.643 |
| 17 | 9.507 | 1.151 | −20.207 | 3.425 | −16.509 | 5.709 | −20.566 | 7.172 | −13.174 | 3.424 | −13.251 | 7.431 |
| 18 | 9.052 | 1.777 | −22.777 | 2.869 | −16.113 | 7.036 | −21.350 | 9.663 | −14.218 | 4.256 | −15.138 | 8.915 |
| 19 | 8.665 | 1.381 | −24.067 | 3.055 | −16.612 | 5.126 | −20.021 | 8.048 | −15.010 | 4.386 | −14.921 | 8.406 |
| 20 | 8.945 | 1.941 | −25.471 | 2.964 | −16.506 | 5.797 | −22.069 | 7.765 | −16.208 | 5.254 | −15.674 | 10.655 |
| 21 | 9.589 | 1.467 | −27.741 | 3.314 | −16.978 | 5.368 | −22.585 | 7.969 | −16.139 | 5.588 | −15.557 | 11.036 |

TABLE 33B
Mean Oxygen Saturation.

| | | | | | Bleo + Compound A | | Bleo + Compound A | | Bleo + Compound A | | Bleo + Pirfenidone | |
|---|---|---|---|---|---|---|---|---|---|---|---|---|
| | Vehicle | | Bleo + Saline | | (1 mg/kg) | | (3 mg/kg) | | (10 mg/kg) | | (300 mg/kg) | |
| Day | Mean | SD | Mean | SD | Mean | SD | Mean | SD | Mean | SD | Mean | SD |
| 7 | 95.86667 | 0.90037 | 85.01667 | 3.673917 | 80.6 | 0.451664 | 82.08333 | 1.188977 | 82.75 | 2.206128 | 81.08333 | 0.495648 |
| 9 | 95.23333 | 0.868716 | 83.2 | 1.060189 | 84.25 | 0.234521 | 83.76667 | 1.242041 | 84.06667 | 1.148332 | 84.55 | 1.489631 |
| 11 | 95.2 | 0.745654 | 84.68333 | 2.492723 | 87.16667 | 0.588784 | 85.05 | 1.082128 | 86.86667 | 0.804156 | 83.31667 | 1.062858 |
| 13 | 94.91667 | 0.82805 | 86.9 | 2.132604 | 88.03333 | 0.68313 | 85.66667 | 0.873308 | 87.5 | 1.256981 | 84.28333 | 0.591326 |
| 15 | 95.61667 | 0.949561 | 85.46667 | 1.781759 | 88.58333 | 2.995608 | 85.96667 | 1.624397 | 88.86667 | 1.330664 | 87.03333 | 1.264384 |
| 17 | 95 | 0.843801 | 85.6 | 3.787875 | 88.48333 | 2.584892 | 89.18333 | 1.098029 | 89.23333 | 1.143095 | 87.08333 | 1.012752 |
| 19 | 94.68333 | 0.495648 | 83.5 | 1.836301 | 89.86667 | 1.962312 | 89.6 | 1.439444 | 92.21667 | 1.505213 | 89.13333 | 0.560952 |
| 21 | 95.46667 | 0.393277 | 84.83333 | 2.228602 | 87.66667 | 2.768875 | 89.88333 | 1.926049 | 93.58333 | 1.07966 | 89.18333 | 0.842417 |

TABLE 33C
Ashcroft Scoring.

| | | | | | | Bleo + Compound A | | | Bleo + Compound A | | | Bleo + Compound A | | | Bleo + Pirfenidone | | |
|---|---|---|---|---|---|---|---|---|---|---|---|---|---|---|---|---|---|
| Vehicle | | | Bleo + Saline | | | (1 mg/kg) | | | (3 mg/kg) | | | (10 mg/kg) | | | (300 mg/kg) | | |
| Mean | Min | Max | Mean | Min | Max | Mean | Min | Max | Mean | Min | Max | Mean | Min | Max | Mean | Min | Max |
| 1.875 | 1 | 3 | 6.5 | 5 | 8 | 5.25 | 4 | 7 | 4.416 | 3 | 5 | 3.708 | 2 | 5 | 5.125 | 4 | 6 |

TABLE 33D
Hydroxyproline Levels.

| | | | | Bleo + Compound A | | Bleo + Compound A | | Bleo + Compound A | | Bleo + Pirfenidone | |
|---|---|---|---|---|---|---|---|---|---|---|---|
| Vehicle | | Bleo + Saline | | (1 mg/kg) | | (3 mg/kg) | | (10 mg/kg) | | (300 mg/kg) | |
| Mean | SD | Mean | SD | Mean | SD | Mean | SD | Mean | SD | Mean | SD |
| 0.041888 | 0.001925 | 0.060525 | 0.003911 | 0.052813 | 0.00536 | 0.054175 | 0.003479 | 0.046275 | 0.002608 | 0.055 | 0.002512 |

TABLE 33E
Functional Lung Volume.

| | | | | Bleo + Compound A | | Bleo + Compound A | | Bleo + Compound A | | Bleo + Pirfenidone | |
|---|---|---|---|---|---|---|---|---|---|---|---|
| Vehicle | | Bleo + Saline | | (1 mg/kg) | | (3 mg/kg) | | (10 mg/kg) | | (300 mg/kg) | |
| Mean | SD | Mean | SD | Mean | SD | Mean | SD | Mean | SD | Mean | SD |
| 591.9113 | 48.39352 | 296.5188 | 60.00858 | 283.165 | 68.91784 | 343.5975 | 40.26114 | 452.7838 | 51.38799 | 445.7025 | 64.96773 |

### Example 122: Systemic Lupus Erythematosus in Animals Treated with a BET Inhibitor and Vehicle

The efficacy of Compound A in treating SLE was evaluated using MRL/MpJ-Fas^{lpr} mice. Lupus develops spontaneously in MRL/MpJ-Fas^{lpr} mice starting around 11 weeks and this model system can be used to investigate the efficacy of a treatment on lupus.

Experimental Method G: SLE in Animals Treated with a BET Inhibitor and Vehicle.

Female MRL/MpJ-Fas^{lpr} mice (The Jackson Laboratory) were used to assess the efficacy of Compound A in treating lupus. Starting when animals were 10 weeks of age, protein concentration in urine was measured using urine dipsticks. At 11 weeks, animals were assigned to treatment groups (Table 34) to achieve a similar mean weight and proteinuria across all treatment groups.

TABLE 34
Lupus study treatment groups

| | | | Dose | | | Volume | |
|---|---|---|---|---|---|---|---|
| Group | n | Treatment | (mg/kg) | Route | Frequency | (mL/kg) | Purpose |
| 1 | 15 | Vehicle | — | PO | QD | 5 | Negative |
| | | | | | | | control |
| 2 | 14 | Compound A | 1 | PO | QD | 5 | Test |
| 3 | 14 | Compound A | 3 | PO | QD | 5 | Test |
| 4 | 14 | Compound A | 10 | PO | QD | 5 | Test |
| 5 | 10 | Cyclophos- | 50 | IP | Once a | 5 | Positive |
| | | phamide | | | week | | control |

At the start of the study, each group in the study had two animals that were 9 weeks old, and all others were 10 weeks old. Dosing of all animals with the test compounds began when the older animals were 11 weeks old. Starting on Day 1, in Groups 1-4, vehicle or Compound A was administered per os (PO) every other day (QD) as described above in Table 33. For Group 5, cyclophosphamide was administered by intraperitoneal injection (IP) once a week. Dosing was at a consistent time each dosing day throughout the study.

Compound A may be formulated as illustrated in Experimental Method A (see Table 2E.). Compound A was prepared in a 5% DMSO/15% PEG 400/80% [10% E-TPGS in Purified Water] formulation. A stock solution of Compound A in DMSO (stock solution concentration of Compound A of 40 mg/mL) was prepared and aliquots for weekly preparations were frozen at −20° C. On each preparation day, a stock solution aliquot was thawed and diluted with other vehicle components to the desired concentration. Formulated Compound A and vehicle were at room temperature for dosing. Cyclophosphamide was dissolved in room temperature PBS to the dosing concentration.

The study began when the older animals were 11 weeks old, and concluded when the older animals were 19 weeks old. Body weight was recorded once a week, with the exception of two weight measurements taken the first week of the study (when the older animals were 11 weeks old). Proteinuria was measured in the urine of animals with urine dipsticks once a week, starting when the older animals were 10 weeks old. Concentrations of anti-dsDNA antibodies or similar autoimmune antibodies were measured in serum by ELISA. Blood samples were taken retro-orbitally at the start of the study to establish a baseline (15 animals, 3 per group), and when the older animals were 16 weeks (all animals) and when animals were 19 weeks (all animals) at the conclusion of the study. Blood urea nitrogen (BUN) was measured in all animals at study termination in blood samples. After blood collection, animals were euthanized, perfused with PBS, and the left kidneys were collected and weighed and fixed in 10% buffered formalin. Left kidneys were then processed to a formalin-fixed, paraffin-embedded (FFPE) block, and histology was analyzed in Periodic Acid-Schiff-stained sections (1 section per slide), using the criteria described in Alperovich et al., Lupus, 16:18-24 (2007). Right kidneys were weighed and snap-frozen at −80° C. Spleens were also collected and weighed. SLE in animals treated with a BET inhibitor and vehicle.

A mouse model of lupus, as described above, was used to assess the efficacy of Compound A in treating lupus.

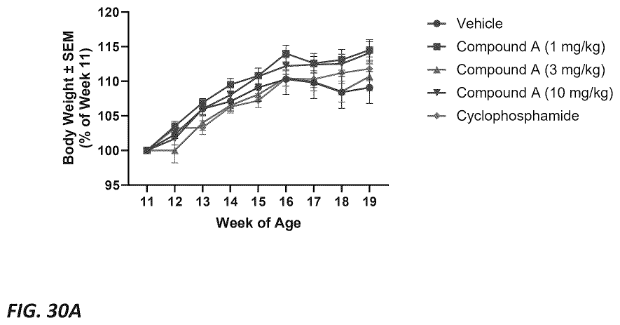

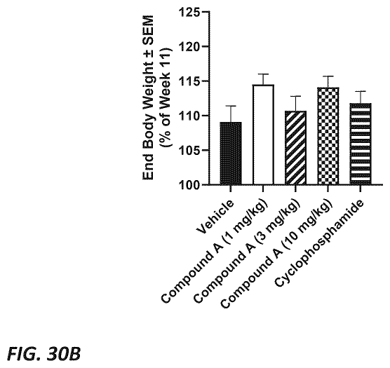

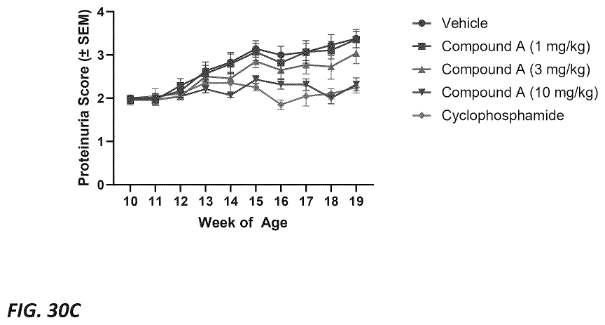

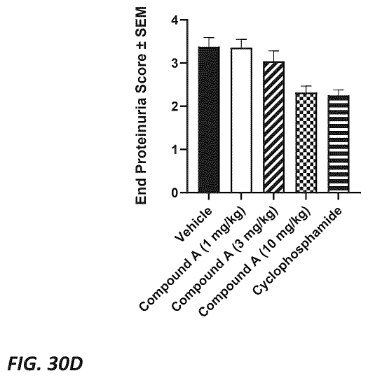

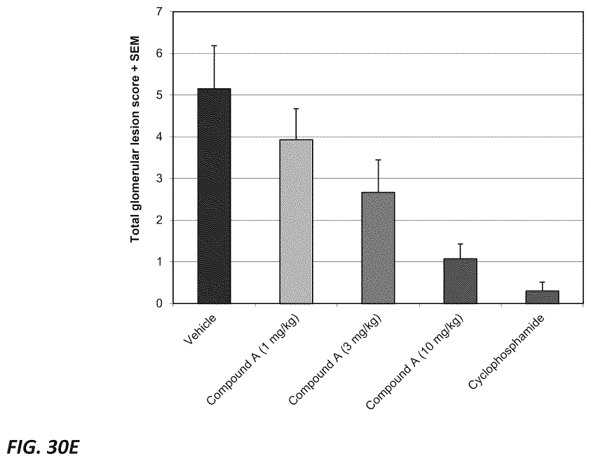

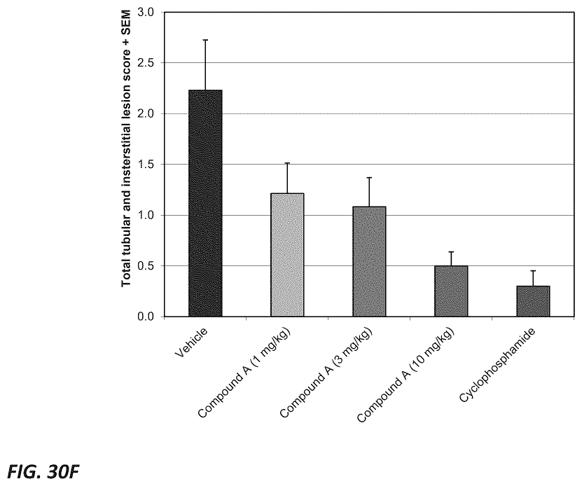

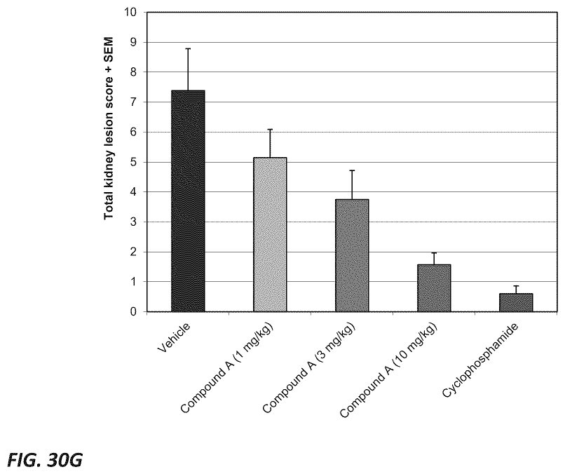

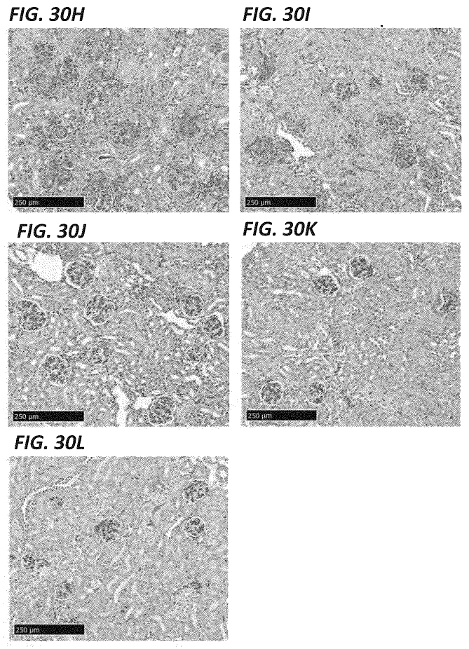

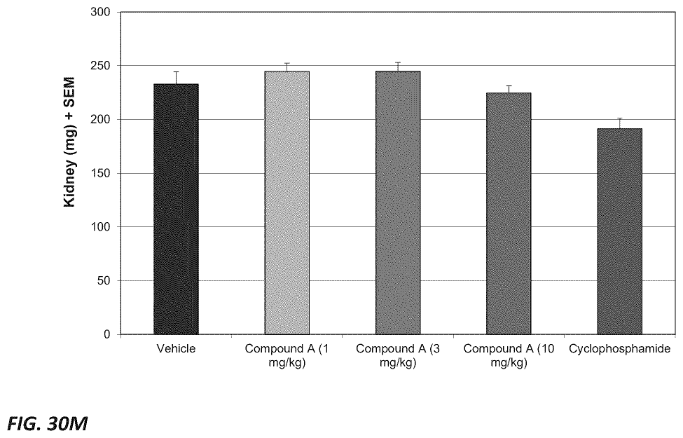

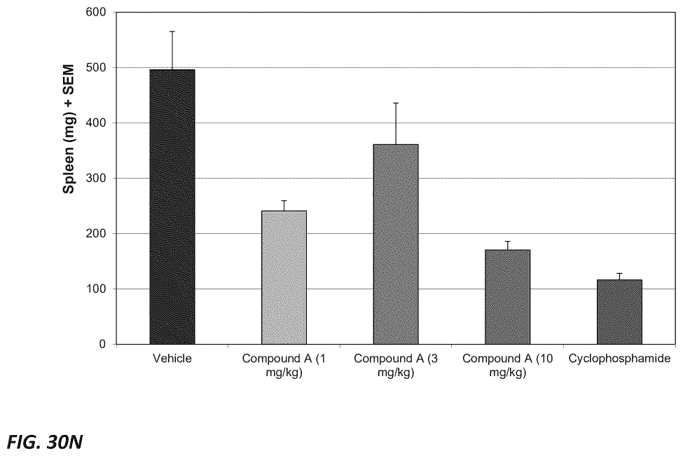

FIG. 30A and Table 35A depict the mean change in body weight over the course of the study. FIG. 30B and Table 35B provide the mean relative end body weight. Vehicle treated animals (Group 1) exhibited weight gain over the first five weeks of the study (through Week 16), but then exhibited a slight decline and plateau in mean body weight gain. Notably, animals in Group 2 and 4 (Compound A 1 mg/kg and 10 mg/kg, respectively) both exhibited a relatively constant weight gain over the entire course ofthe study and exhibited improved weight gains relative to the control groups (Group 1-vehicle and Group 5-cyclophosphamide). Animals receiving Compound A (3 mg/kg) also demonstrated a constant weight gain through Week 16, and exhibited slightly increased weight gains at Week 19 relative to Group 1 (vehicle control).

TABLE 35A
Mean body weight over the study.

| | Mean body weight (g) ± SEM | | | | | | | | |
|---|---|---|---|---|---|---|---|---|---|
| | Week | Week | Week | Week | Week | Week | Week | Week | Week |
| Group | 11 | 12 | 13 | 14 | 15 | 16 | 17 | 18 | 19 |
| Vehicle | 35.1 ± 0.9 | 35.9 ± 0.8 | 37.1 ± 0.8 | 37.4 ± 0.7 | 37.8 ± 0.9 | 38.8 ± 0.7 | 38.7 ± 0.8 | 38.8 ± 1.0 | 39.1 ± 1.3 |
| Compound A | 35.2 ± 0.9 | 36.4 ± 0.8 | 37.6 ± 0.9 | 38.5 ± 0.9 | 38.9 ± 0.8 | 40.0 ± 0.8 | 39.5 ± 0.8 | 39.7 ± 0.7 | 40.2 ± 0.8 |
| (1 mg/kg) | | | | | | | | | |
| Compound A | 35.0 ± 0.7 | 35.0 ± 0.8 | 36.5 ± 0.5 | 37.4 ± 0.7 | 37.9 ± 0.7 | 38.7 ± 0.7 | 38.6 ± 0.8 | 38.1 ± 0.7 | 38.6 ± 1.0 |
| (3 mg/kg) | | | | | | | | | |
| Compound A | 35.1 ± 0.7 | 35.6 ± 0.6 | 37.1 ± 0.6 | 37.8 ± 0.6 | 38.8 ± 0.7 | 39.3 ± 0.7 | 39.3 ± 0.6 | 39.4 ± 0.6 | 39.9 ± 0.7 |
| (10 mg/kg) | | | | | | | | | |
| Cyclophosphamide | 35.0 ± 1.2 | 36.2 ± 1.2 | 36.1 ± 1.1 | 37.2 ± 1.0 | 37.5 ± 1.0 | 38.6 ± 1.0 | 38.6 ± 1.2 | 38.9 ± 1.3 | 39.1 ± 1.3 |

TABLE 35B
Mean relative end body weight (% of Week 11).

| | Relative end | |
|---|---|---|
| | body weight | p value |
| Group | (% of Week 11) ± SEM | (versus Vehicle) |
| Vehicle | 109.1% ± 2.3% | — |
| Compound A (1 mg/kg) | 114.5% ± 1.5% | 0.060 |
| Compound A (3 mg/kg) | 110.7% ± 2.1% | 0.625 |
| Compound A (10 mg/kg) | 114.4% ± 1.6% | 0.082 |
| Cyclophosphamide | 111.8% ± 1.7% | 0.378 |

FIG. 30C and Table 36A show mean proteinuria scores, measured weekly, from Week 10 through Week 19. FIG. 30D and Table 3 GB show the mean end proteinuria scores. As expected, vehicle-treated animals (Group 1) exhibited a steady increase in proteinuria levels over the course of the study. Animals receiving the low dose of Compound A exhibited proteinuria levels relatively similar to Group 1 (vehicle control). Proteinuria levels in the two higher doses for Compound A (3 mg/kg and 10 mg/kg) were decreased relative to Group 1 and Group 2 (Compound A 1 mg/kg), with the lowest proteinuria levels in the 10 mg/kg group, indicating that Compound A reduced proteinuria levels in a dose-dependent manner. At Week 19, proteinuria levels for animals receiving Compound A (10 mg/kg) were similar to proteinuria levels for animals receiving cyclophosphamide, and about 74.8% lower than the vehicle-treated group (Group 1) (P<0.001).

TABLE 36A
Mean proteinuria scores over the study.

| | Mean proteinuria score ± SEM | | | | | | | | | |
|---|---|---|---|---|---|---|---|---|---|---|
| | Week | Week | Week | Week | Week | Week | Week | Week | Week | Week |
| Group | 10 | 11 | 12 | 13 | 14 | 15 | 16 | 17 | 18 | 19 |
| Vehicle | 2.00 ± 0.07 | 2.00 ± 0.11 | 2.17 ± 0.15 | 2.63 ± 0.21 | 2.83 ± | 3.14 ± | 3.00 ± | 3.07 ± | 3.23 ± | 3.38 ± |
| | | | | | 0.23 | 0.19 | 0.20 | 0.26 | 0.24 | 0.21 |
| Compound A | 1.96 ± 0.08 | 1.96 + 0.11 | 2.29 ± 0.16 | 2.57 ± 0.19 | 2.79 ± | 3.07 ± | 2.82 ± | 3.07 ± | 3.11± | 3.36 ± |
| (1 mg/kg) | | | | | 0.23 | 0.20 | 0.22 | 0.20 | 0.20 | 0.19 |
| Compound A | 1.96 ± 0.06 | 1.96 ± 0.13 | 2.04 ± 0.07 | 2.50 ± 0.19 | 2.46 ± | 2.85 ± | 2.65 ± | 2.77 ± | 2.73 ± | 3.04 ± |
| (3 mg/kg) | | | | | 0.12 | 0.15 | 0.19 | 0.21 | 0.29 | 0.24 |
| Compound A | 1.96 ± 0.11 | 1.96 ± 0.11 | 2.04 ± 0.08 | 2.21 ± 0.09 | 2.07 ± | 2.43 ± | 2.32 ± | 2.32 ± | 2.00 ± | 2.32 ± |
| (10 mg/kg) | | | | | 0.05** | 0.09** | 0.11** | 0.13* | 0.13*** | 0.15*** |
| Cyclophosphamide | 2.00 ± 0.07 | 2.05 ± 0.17 | 2.10 ± 0.10 | 2.35 ± 0.22 | 2.35 ± | 2.25 ± | 1.85 ± | 2.05 ± | 2.10 ± | 2.25 ± |
| | | | | | 0.21 | 0.08** | 0.11*** | 0.23* | 0.12** | 0.13*** |

*p < 0.05,
**p < 0.01,
***p < 0.001

TABLE 36B
Mean end proteinuria scores.

| | End proteinuria | p value |
|---|---|---|
| Group | score ± SEM | (versus Vehicle) |
| Vehicle | 3.38 ± 0.21 | — |
| Compound A (1 mg/kg) | 3.36 ± 0.19 | 0.958 |
| Compound A (3 mg/kg) | 3.04 ± 0.24 | 0.277 |
| Compound A (10 mg/kg) | 2.32 ± 0.15 | <0.001 |
| Cyclophosphamide | 2.25 ± 0.13 | <0.001 |

FIGS. 30E-30G depict lesion scoring for animals in the study. FIG. 30E and Table 37A show mean total glomerular lesion scores. Animals in Group 1 (vehicle control) exhibited a mean total glomerular lesion score at Week 19 of 5.2. Compound A effectively reduced mean total glomerular lesion scores in a dose-dependent manner, with mean scores at Week 19 of about 3.9 (1 mg/kg), about 2.7 (3 mg/kg), and about 1.1 (10 mg/kg). Animals receiving cyclophosphamide had a mean total glomerular lesion score of 0.3. FIG. 30F and Table 37B show the mean total tubular and interstitial lesion score for animals. Animals in Group 1 (vehicle control) exhibited a mean total tubular and interstitial lesion score of about 2.2 at Week 19. Compound A effectively reduced mean total tubular and interstitial lesion scores in a dose-dependent manner, with mean scores at Week 19 of about 1.2 (1 mg/kg), about 1.1 (3 mg/kg), and about 0.5 (10 mg/kg). Animals receiving cyclophosphamide had a mean total tubular and interstitial lesion score of about 0.3. FIG. 30G and Table 36C show the mean total kidney lesion score for animals. Animals in Group 1 (vehicle control) exhibited a mean total kidney lesion score of about 7.4 at Week 19. Compound A effectively reduced mean total kidney lesion scores in a dose-dependent manner, with mean scores at Week 19 of about 5.1 (1 mg/kg), about 3.8 (3 mg/kg), and about 1.6 (10 mg/kg). Animals receiving cyclophosphamide had a mean total kidney lesion score of about 0.6.

TABLE 37A
Mean Total Glomerular Lesion Scores.

| Group | Total Glomerular Lesion | SEM |
|---|---|---|
| Vehicle | 5.2 | 1.0 |
| Compound A (1 mg/kg) | 3.9 | 0.7 |
| Compound A (3 mg/kg) | 2.7 | 0.8 |
| Compound A (10 mg/kg) | 1.1 | 0.4 |
| Cyclophosphamide | 0.3 | 0.2 |

TABLE 37B
Mean Total Tubular and Interstitial Lesion Scores.

| Group | Total Tubular and Interstitial Lesion | SEM |
|---|---|---|
| Vehicle | 2.2 | 0.5 |
| Compound A (1 mg/kg) | 1.2 | 0.3 |
| Compound A (3 mg/kg) | 1.1 | 0.3 |
| Compound A (10 mg/kg) | 0.5 | 0.1 |
| Cyclophosphamide | 0.3 | 0.2 |

TABLE 37C
Mean Total Kidney Lesion Scores.

| Group | Total Glomerular Lesion | SEM |
|---|---|---|
| Vehicle | 7.4 | 1.4 |
| Compound A (1 mg/kg) | 5.1 | 0.9 |
| Compound A (3 mg/kg) | 3.8 | 1.0 |
| Compound A (10 mg/kg) | 1.6 | 0.4 |
| Cyclophosphamide | 0.6 | 0.3 |

As can also be visualized from the illustrative histology images provided in FIGS. 30H-30L, increasing the dosage of Compound A results in a dose dependent improvement in disease suppression, with animals receiving the higher 10 mg/kg dose having histology that is similar in appearance to the positive control cyclophosphamide. The results of kidney histological analysis were as expected for vehicle-treated mice.

FIGS. 30M and 30N show mean kidney weight and mean spleen weight, respectively, at the conclusion of the study at Week 19 (see also Tables 37D and 37E, respectively). Kidney weights (FIG. 30M and Table 37D) were relatively similar for all treatment groups, with animals in the Compound A (10 mg/kg) and cyclophosphamide treatment groups exhibiting slightly decreased kidney weights relative to controls. Animals receiving Compound A (all tested doses) and cyclophosphamide exhibited decreased spleen weights relative to vehicle controls (FIG. 30N and Table 37E).

TABLE 37D
Mean Kidney Weight.

| Group | Mean Weight (mg) | SEM |
|---|---|---|
| Vehicle | 233 | 11 |
| Compound A (1 mg/kg) | 245 | 8 |
| Compound A (3 mg/kg) | 245 | 8 |
| Compound A (10 mg/kg) | 225 | 7 |
| Cyclophosphamide | 191 | 10 |

TABLE 37E
Mean Spleen Weight.

| Group | Mean Weight (mg) | SEM |
|---|---|---|
| Vehicle | 496 | 69 |
| Compound A (1 mg/kg) | 241 | 19 |
| Compound A (3 mg/kg) | 361 | 75 |
| Compound A (10 mg/kg) | 170 | 16 |
| Cyclophosphamide | 116 | 12 |

TABLE 37F
Mean Serum BUN (mg/dL).

| Group | BUN serum concentration (mg/dL) | SEM |
|---|---|---|
| Vehicle | 58.4 | 21.3 |
| Compound A (1 mg/kg) | 18.0 | 2.4 |
| Compound A (3 mg/kg) | 16.8 | 4.4 |
| Compound A (10 mg/kg) | 11.0 | 1.2 |
| Cyclophosphamide | 11.0 | 1.6 |

TABLE 37G
Mean anti-dsDNA Antibodies (units/mL) in serum.

| | Week 11 | | Week 16 | | Week 19 | |
|---|---|---|---|---|---|---|
| | Anti- | | Anti- | | Anti- | |
| | dsDNA | | dsDNA | | dsDNA | |
| | Antibodies | | Antibodies | | Antibodies | |
| Group | (units/mL) | SEM | (units/mL) | SEM | (units/mL) | SEM |
| Vehicle | | | 147 | 38 | 183 | 39 |
| Compound A (1 mg/kg) | | | 240 | 101 | 149 | 65 |
| Compound A (3 mg/kg) | | | 112 | 49 | 94 | 46 |
| Compound A (10 mg/kg) | | | 47 | 8 | 57 | 16 |
| Cyclophosphamide | | | 28 | 6 | 23 | 2 |
| Baseline | 68 | 18 | | | | |

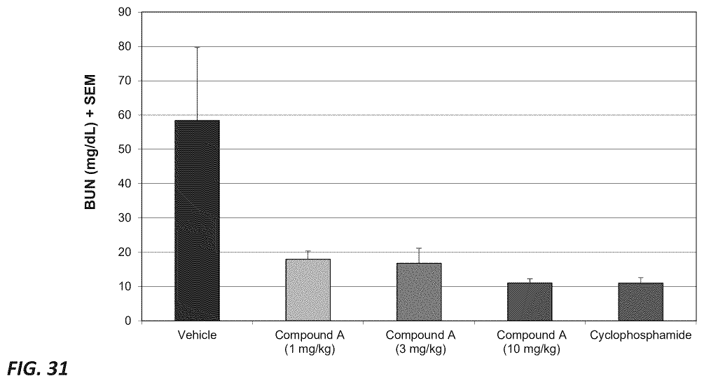

FIG. 31 and Table 37F depict blood urea nitrogen (BUN) concentration (mg/dL) in serum after study completion at Week 19. BUN provides important information about how well kidneys are working. A BUN test measures the amount of urea nitrogen in the blood and indicates how much urea has been processed by the kidneys and is indicative of renal competency. Urea is a waste product that is excreted in urine. High BUN levels as seen with the vehicle group indicate renal impairment which is a primary symptom of SLE. In contrast Compound A was able to reduce BUN in a dose-dependent manner with the highest dose (10 mg/mL) reducing BUN to 11 mg/dL (p=0.030), which is over five times lower than that of the Vehicle treated group (58.4). Cyclophosphamide administration was similar in effect to Compound A (10 mg/mL).

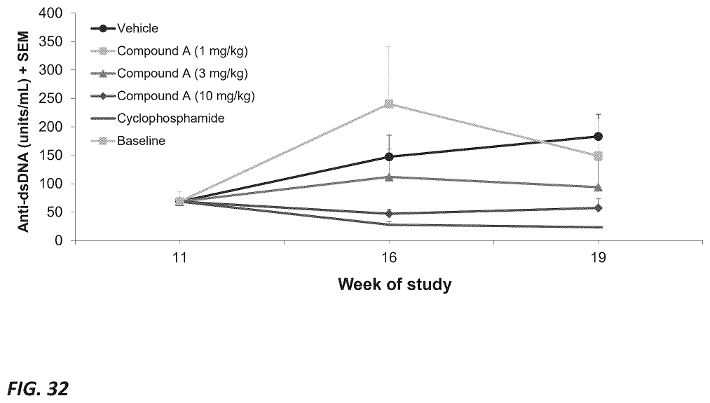

FIG. 32 and Table 37G show Anti-dsDNA antibodies in serum at Weeks 11, 16 and 19. Anti-dsDNA antibody test measures the amount of antibody to double-stranded deoxyribonucleic acid (anti-dsDNA) that may be present in the blood. Anti-dsDNA antibodies are highly specific markers of SLE as these antibodies are not thought to be present in other kinds of autoimmune disorder. It can be seen in FIG. 32 and Table 37G that the levels of Anti-dsDNA antibodies in Vehicle treated animals steadily increase over the period of Week 11 to Week 16 to Week 19. In Contrast Compound A (10 mg/mL) and Cyclophosphamide were effective in reducing levels to well below that seen with the Vehicle administered group both at week 16 and Week 19. Although the lowest concentration of Compound A (1 mg/mL) did not appear to be effective at Week 16 it did appear to be effective in reducing the levels by Week 19. The mid concentration of Compound A (3 mg/mL) was seen to have a positive effect in reducing levels both at Week 16 and at Week 19. Moreover, by Week 19 Compound A was seen to be effective in reducing levels in a dose-dependent manner. So, the BUN and the Anti-dsDNA antibodies data further point to Compound A having the potential to treat lupus effectively and ameliorate slow or arrest lupus associated kidney damage.

Without being bound by any particular theory, Compound A is effective in treating systemic lupus erythematosus, as evidenced by its ability to protect against decreases in weight gain, inhibit proteinuria in a dose-dependent manner, reduce BUN and anti-dsDNA antibodies in a dose-dependent manner, reduce histopathological signs of disease, including glomerular, tubular and interstitial, and kidney lesions, and protect against spleen enlargement/weight gain. Without being bound by any particular theory, given the observed dose-dependent efficacy with the highest dose seen to have the greatest effect in reducing various indicators of lupus and associated kidney disorder it may be that further improvements in treating lupus are achievable by administering doses that are higher than 10 mg/kg.

### Example 123: Impact of BET Inhibitor Formulations and Vehicle on Gastrointestinal Histopathology

Oral pan-BD BET inhibitors are known to negatively impact gastrointestinal mucosa through goblet cell toxicity, leading to related gastrointestinal adverse events in the clinic (Faivre et al., Nature, 578:306-310 (2020)). Therefore, the impact of Compound A on gastrointestinal toxicity and the mucosa was assessed in healthy beagle dogs as part of Dog PK Study #4, outlined in Experimental Method B.

Following completion of Dog PK study #4 on Day 15, animals were euthanized, and the large and small intestines were collected and fixed in 10% neutral buffered formalin. The tissues were then removed from the fixative, processed, embedded in paraffin, sectioned, and stained with hematoxylin and eosin, prior to microscopic examination.

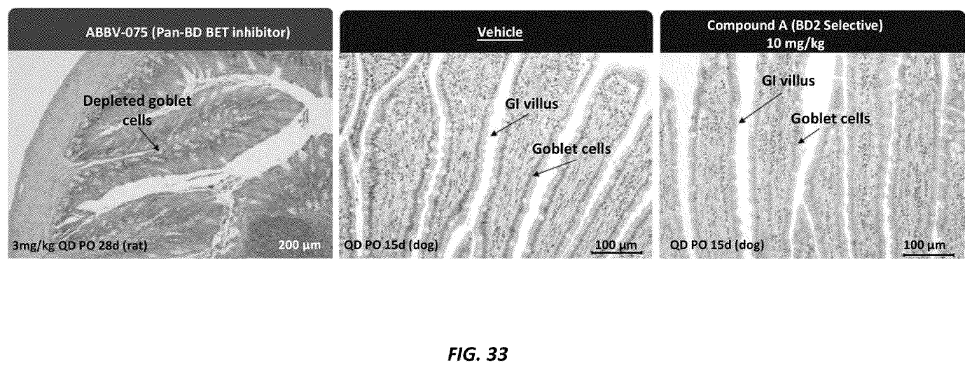

It was found that Compound A (10 mg/kg) had no deleterious effect on gastrointestinal villus morphology or goblet cell number (FIG. 33), whereas a pan-BD BET inhibitor (ABBV-075) depleted goblet cells in gastrointestinal villi at 28 days (Faivre et al., Nature, 578:306-310 (2020); not a head-to-head comparison). Without being bound by any particular theory, Compound A does not appear to negatively impact the gastrointestinal mucosa, unlike pan-BD BET inhibitors.

### Example 124: Impact of a BET Inhibitor and Vehicle on a Model of Experimental Autoimmune Encephalomyelitis

Experimental autoimmune encephalomyelitis (EAE) is the most commonly used mouse model of human multiple sclerosis (MS). One of ordinary skill in the art would be familiar with the EAE model. See, e.g., Constantinescu et al., Br. J. Pharmacol. 164(4):1079-1106 (2011). Because of its many similarities to MS, EAE is used to study the pathogenesis of autoimmunity, CNS inflammation, demyelination, cell trafficking, and tolerance induction. EAE is mediated, at least in part, by CD4+ T cells, CD8+ T cells, and B cells. The EAE model was therefore used to assess the potential impact of Compound A on EAE development and pathogenesis.

### Experimental Method H: EAE in Animals Treated with a BET Inhibitor and Vehicle

Animals were assigned to the groups outlined in Table 38.

TABLE 38
EAE study treatment groups.

| | | | Dose | | | | |
|---|---|---|---|---|---|---|---|
| Group | n | Treatment | (mg/kg) | Route | Frequency | Volume | Purpose |
| 1 | 12 | Vehicle | — | PO | QD | 10 | Negative |
| | | | | | | mL/kg | control |
| 2 | 12 | Compound | 1 | PO | QD | 10 | Test |
| | | A | | | | mL/kg | |
| 3 | 12 | Compound | 3 | PO | QD | 10 | Test |
| | | A | | | | mL/kg | |
| 4 | 12 | Compound | 10 | PO | QD | 10 | Test |
| | | A | | | | mL/kg | |
| 5 | 12 | FTY720* | 3 | PO | QD | 10 | Positive |
| | | | | | | mL/kg | control |

*Also known as fingolimod, approved for the treatment of relapsing, remitting multiple sclerosis (GILENYA ®).

Compound A may be formulated as illustrated in Experimental Method A (see Table 2E.). A 40 mg/mL stock solution of Compound A in DMSO was prepared and aliquots stored at −20° C. On each day of preparation an e.g., 0.75 mL aliquot of thawed stock was diluted with an equal volume of DMSO. The remaining vehicle components were added to provide the highest dose and then diluted with vehicle to provide lower doses. A 30 mg/mL stock solution of FTY720 in ethanol was prepared and stored at −20° C. Daily, the stock solution was diluted with room temperature water to a final dosing concentration of 0.6 mg/mL. Formulated Compound A, FTY720 and vehicle were each at room temperature for dosing.

Female C57B116 mice (9 weeks old) were acclimated for seven days prior to study initiation. On Day −1, animals were assigned to groups in a balanced manner to achieve similar average weights across the different groups. Chronic EAE was induced in female C57BI/6 mice following immunization with an emulsion of MOG3s-ss/CFA on Day 0, followed by an injection of pertussis toxin two hours after the initial immunization. On Day 1, a second injection of pertussis toxin was administered 24 hours after the initial immunization. Mice were injected subcutaneously at two sites on the back with the MOG3s-ss/CFA emulsion (Hooke Kit™ MOG3S-ss/CFA Emulsion PTX). The first injection site was located approximately 1 cm caudal of the neckline (upper back), and the second injection site was approximately 2 cm cranial of the base of the tail (lower back). The injection volume was 0.1 mL for each injection. The pertussis toxin was administered at the described times through intraperitoneal injection at a concentration of 85 ng/dose for both injections.

Body weight was measured three times a week (Monday, Wednesday, and Friday) starting on Day −1. Animals were scored daily for EAE starting on Day 7. EAE scoring was based on the guidelines set forth in Table 39. In-between scores were assigned when the clinical signs fell between two below defined scores.

TABLE 39
EAE scoring criteria.

| Score | Clinical observations |
|---|---|
| 0 | No obvious changes in motor functions |
| | of the mouse in comparison to |
| | non-immunized mice. |
| | When picked up by the tail, the tail |
| | has tension and is erect. Hind legs are |
| | usually spread apart. When the mouse |
| | is walking, there is no gait or head |
| | tilting. |
| 1 | Limp tail. |
| | When the mouse is picked up by the tail, |
| | instead of being erect, the whole tail |
| | drapes over your finger. |
| 2 | Limp tail and weakness of hind legs. |
| | When mouse is picked up by the tail, |
| | legs are not spread apart, but held closer |
| | together. When the mouse is observed |
| | when walking, it has a clearly apparent |
| | wobbly walk. |
| 3 | Limp tail and complete paralysis |
| | of hind legs (most common). |
| | OR |
| | Limp tail with paralysis of one |
| | front and one hind leg. |
| | OR |
| | All of: |
| | Severe head tilting, |
| | Walking only along the edges of the cage, |
| | Pushing against the cage wall, |
| | Spinning when picked up by the tail |
| 4 | Limp tail, complete hind leg and |
| | partial front leg paralysis. |
| | Mouse is minimally moving around the |
| | cage but appears alert and feeding. |
| | Usually, euthanasia is recommended |
| | after the mouse scores level 4 for 2 days. |
| | When the mouse is euthanized because |
| | of severe paralysis, a score of 5 is |
| | entered for that mouse for the rest |
| | of the experiment. |
| 5 | Complete hind and complete front leg |
| | paralysis, no movement around the cage. |
| | OR |
| | Mouse is spontaneously rolling in the cage. |
| | OR |
| | Mouse is found dead due to paralysis. |

On Day 28, all surviving mice except those in the FTY720 group had blood collected via retro-orbital sinus into serum separator tubes. Serum was isolated, frozen on dry ice, and stored at −80° C. for cytokine analysis. Cytokine analysis was performed using Luminex in serum samples, and the cytokines IL-22, IFNγ, TNF, IL-17A, IL-12/IL-23p40 were assessed.

### Impact of Compound a on EAE in Mice

EAE development was evaluated by comparing the clinical EAE readouts for each group relative to the Vehicle group. Animals in the vehicle group developed EAE as expected. The study demonstrated a dose dependent efficacy of Compound A in reducing EAE severity.

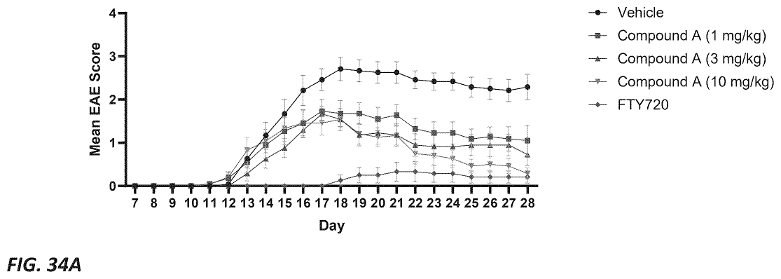

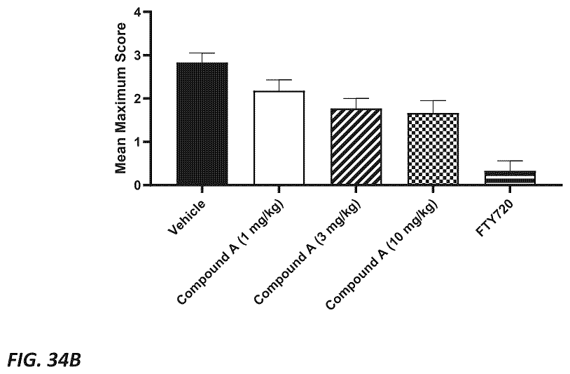

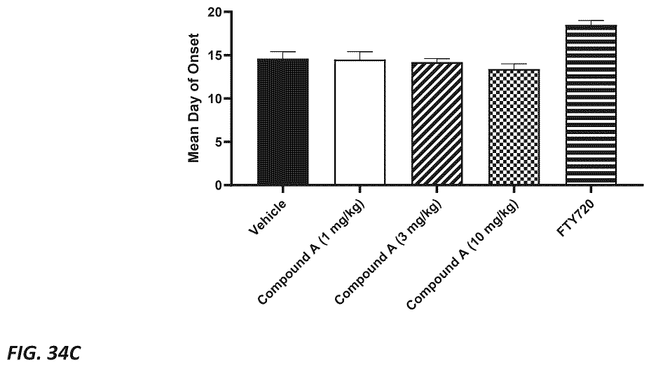

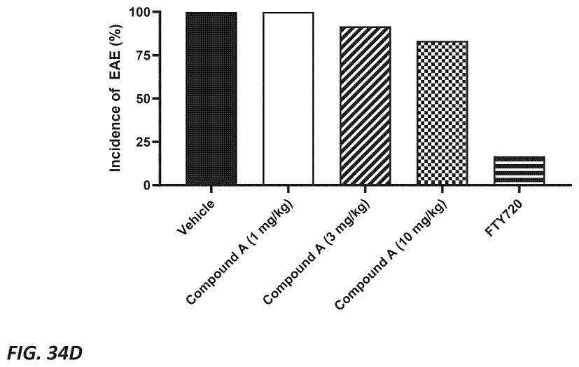

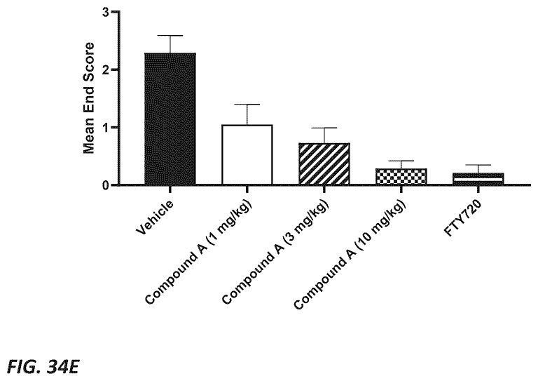

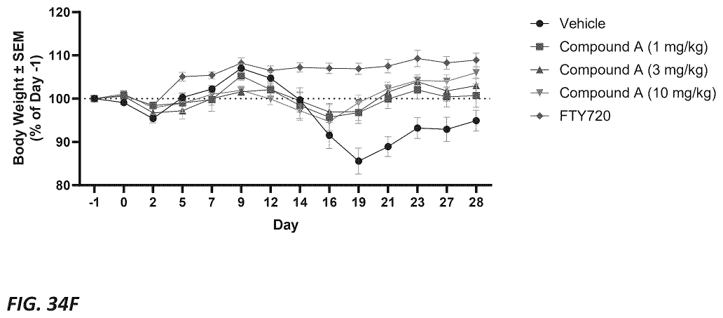

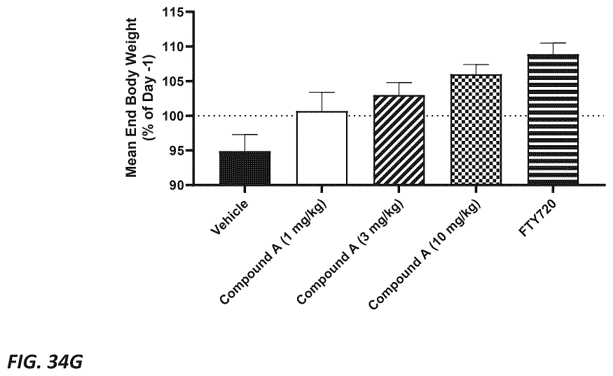

EAE scoring over the course of the study for all groups is shown in Table 40A and FIG. 34A. Table 40B shows the mean EAE maximum score (FIG. 34B), mean day of onset of EAE (FIG. 34C), median day of onset of EAE, percent incidence of EAE (FIG. 34D), and the mean end EAE score for animals (FIG. 34E). Vehicle-treated animals began exhibiting clinical EAE scores starting on day 12, which continued until scores peaked at day 18, and then EAE scores remained relatively constant over the remainder of the study, ending with a mean EAE score of 2.29. All three groups receiving Compound A exhibited generally similar onset of EAE, however, EAE scores for all animals receiving Compound A peaked around day 17, and decreased in severity to final mean end EAE scores in a dose-dependent manner. Animals receiving Compound A (1 mg/kg, 3 mg/kg and 10 mg/kg) exhibited significantly reduced mean maximum scores in a dose dependent manner relative to vehicle, and all animals receiving Compound A exhibited significantly reduced mean end scores in a dose dependent manner relative to vehicle with the end score of the 10 mg/ml group being similar to that for FTY720. The p value for both the end score and the maximum score, e.g., for the 10 mg/kg group compared to vehicle was <0.001 (see Table 40B).

TABLE 40A
EAE scores.

| | | Day | | | | | | | | | | |
|---|---|---|---|---|---|---|---|---|---|---|---|---|
| Group | Score | 7 | 8 | 9 | 10 | 11 | 12 | 13 | 14 | 15 | 16 | 17 |
| 1 | Mean | 0.00 | 0.00 | 0.00 | 0.00 | 0.00 | 0.04 | 0.63 | 1.17 | 1.67 | 2.21 | 2.46 |
| | SEM | 0.00 | 0.00 | 0.00 | 0.00 | 0.00 | 0.04 | 0.22 | 0.31 | 0.34 | 0.35 | 0.26 |
| 2 | Mean | 0.00 | 0.00 | 0.00 | 0.00 | 0.05 | 0.18 | 0.55 | 0.95 | 1.27 | 1.45 | 1.73 |
| | SEM | 0.00 | 0.00 | 0.00 | 0.00 | 0.05 | 0.14 | 0.25 | 0.30 | 0.31 | 0.32 | 0.28 |
| 3 | Mean | 0.00 | 0.00 | 0.00 | 0.00 | 0.00 | 0.00 | 0.29 | 0.63 | 0.88 | 1.29 | 1.67 |
| | SEM | 0.00 | 0.00 | 0.00 | 0.00 | 0.00 | 0.00 | 0.17 | 0.22 | 0.22 | 0.18 | 0.18 |
| 4 | Mean | 0.00 | 0.00 | 0.00 | 0.00 | 0.04 | 0.21 | 0.83 | 1.04 | 1.33 | 1.46 | 1.46 |
| | SEM | 0.00 | 0.00 | 0.00 | 0.00 | 0.04 | 0.13 | 0.29 | 0.27 | 0.30 | 0.29 | 0.28 |
| 5 | Mean | 0.00 | 0.00 | 0.00 | 0.00 | 0.00 | 0.00 | 0.00 | 0.00 | 0.00 | 0.00 | 0.00 |
| | SEM | 0.00 | 0.00 | 0.00 | 0.00 | 0.00 | 0.00 | 0.00 | 0.00 | 0.00 | 0.00 | 0.00 |
| | | Day | | | | | | | | | | |
| Group | Score | 18 | 19 | 20 | 21 | 22 | 23 | 24 | 25 | 26 | 27 | 28 |
| 1 | Mean | 2.71 | 2.67 | 2.63 | 2.63 | 2.46 | 2.42 | 2.42 | 2.29 | 2.25 | 2.21 | 2.29 |
| | SEM | 0.27 | 0.26 | 0.25 | 0.25 | 0.21 | 0.20 | 0.20 | 0.23 | 0.24 | 0.26 | 0.30 |
| 2 | Mean | 1.68 | 1.68 | 1.55 | 1.64 | 1.32 | 1.23 | 1.23 | 1.09 | 1.14 | 1.09 | 1.05 |
| | SEM | 0.30 | 0.25 | 0.27 | 0.24 | 0.25 | 0.26 | 0.26 | 0.23 | 0.23 | 0.28 | 0.35 |
| 3 | Mean | 1.54 | 1.18 | 1.23 | 1.18 | 0.95 | 0.91 | 0.91 | 0.95 | 0.95 | 0.95 | 0.73 |
| | SEM | 0.19 | 0.25 | 0.26 | 0.26 | 0.26 | 0.26 | 0.30 | 0.24 | 0.27 | 0.25 | 0.26 |
| 4 | Mean | 1.54 | 1.21 | 1.13 | 1.17 | 0.75 | 0.71 | 0.63 | 0.46 | 0.50 | 0.46 | 0.29 |
| | SEM | 0.26 | 0.24 | 0.22 | 0.22 | 0.24 | 0.23 | 0.21 | 0.16 | 0.18 | 0.17 | 0.13 |
| 5 | Mean | 0.13 | 0.25 | 0.25 | 0.29 | 0.33 | 0.29 | 0.29 | 0.21 | 0.21 | 0.21 | 0.21 |
| | SEM | 0.13 | 0.18 | 0.18 | 0.22 | 0.23 | 0.20 | 0.20 | 0.14 | 0.14 | 0.14 | 0.14 |

TABLE 40B
EAE score details.

| | Mean Maximum | Mean Day of | Median Day of | | Mean End Score |
|---|---|---|---|---|---|
| Group | Score (SEM) | Onset (SEM) | Onset | Incidence of EAE (%) | (SEM) |
| 1 | 2.83 (0.22) | 14.6 (0.8) | 13.0 | 100 | 2.29 (0.30) |
| 2 | 2.18 (0.25)* | 14.5 (0.9) | 13.0 | 100 | 1.05 (0.35)* |
| 3 | 1.77 (0.23)*** | 14.2 (0.4) | 14.0 | 91.7 | 0.73 (0.26)** |
| 4 | 1.67 (0.28)*** | 13.4 (0.6) | 13.0 | 83.3 | 0.29 (0.13)*** |
| 5 | 0.33 (0.23)*** | 18.5 (0.5) | >28.0 | 16.7*** | 0.21 (0.14)*** |

*p < 0.05,
**p < 0.01,
***p < 0.001;
statistical analyses are relative to the vehicle group (Group 1).

The mean day of onset of EAE was similar for animals receiving Compound A and vehicle, as was the median day of onset of EAE. Although 100% of animals receiving the lowest dose of Compound A had EAE, not all animals receiving Compound A at the two higher doses, 3 mg/kg and 10 mg/kg, had EAE (91.7% incidence rate and 83.3% incidence rate, respectively).

All animals experienced a slight decrease in body weight when measured at day 2, and then body weights increased until day 9 (FIG. 34F). Starting on day 9, animals receiving vehicle experienced a sharp and constant decline in body weight change until day 19, which was followed by a slight recovery in body weight. Animals receiving vehicle had a relative end weight that was 94.9% of their weight taken on Day −1. Animals receiving Compound A noted changes in body weight similar to vehicle over the first week of the study, but notably did not experience a sharp decline in body weight at any point in the study. Instead, animals receiving Compound A exhibited slight decreases in body weight that all recovered to at least the same body weight as was taken on Day −1 by the end of the study. The end relative body weight of all animals receiving Compound A was significantly increased relative to the vehicle treated group (Table 41B, FIG. 34G). Animals receiving Compound A exhibited a dose-dependent increase in body weight gain, with animals receiving a dose of 1 mg/kg of Compound A having a relative end weight of 100.7% of the mean Day −1 weight, animals receiving a dose of 3 mg/kg of Compound A having a relative end weight of 103% of the mean Day −1 weight, and animals receiving a dose of 10 mg/kg of Compound A having a relative end weight of 106% of the mean Day −1 weight. The relative end weight for animals receiving 10 mg/kg of Compound A was similar to the relative end weight of animals receiving the positive control, FTY720 (see FIG. 34F and Table 41A).

TABLE 41A
Body weight % change.

| | | Day | | | | | | | | | | | | | |
|---|---|---|---|---|---|---|---|---|---|---|---|---|---|---|---|
| Group | | −1 | 0 | 2 | 5 | 7 | 9 | 12 | 14 | 16 | 19 | 21 | 23 | 27 | 28 |
| 1 | Mean Body Weight | 100.0 | 99.1 | 95.4 | 100.3 | 102.2 | 107.0 | 104.7 | 99.7 | 91.5 | 85.6 | 88.9 | 93.2 | 92.9 | 94.9 |
| | (% of Day −1) | | | | | | | | | | | | | | |
| | SEM | 0.0 | 0.5 | 1.0 | 1.1 | 0.8 | 1.0 | 1.6 | 2.8 | 3.0 | 3.0 | 2.3 | 2.4 | 2.8 | 2.4 |
| 2 | Mean Body Weight | 100.0 | 100.7 | 98.4 | 99.0 | 99.8 | 105.3 | 102.1 | 98.4 | 95.7 | 96.8 | 99.9 | 102.0 | 100.4 | 100.7 |
| | (% of Day −1) | | | | | | | | | | | | | | |
| | SEM | 0.0 | 0.6 | 0.8 | 2.2 | 2.7 | 1.0 | 1.8 | 3.0 | 3.2 | 2.5 | 2.2 | 2.1 | 2.3 | 2.7 |
| 3 | Mean Body Weight | 100.0 | 100.6 | 96.7 | 97.2 | 100.0 | 101.6 | 102.1 | 99.5 | 96.9 | 97.0 | 101.5 | 103.9 | 101.8 | 103.0 |
| | (% of Day −1) | | | | | | | | | | | | | | |
| | SEM | 0.0 | 0.4 | 1.7 | 1.9 | 1.5 | 0.9 | 1.5 | 2.1 | 1.7 | 2.1 | 1.8 | 1.6 | 2.4 | 1.8 |
| 4 | Mean Body Weight | 100.0 | 101.1 | 97.9 | 98.9 | 101.0 | 101.9 | 99.9 | 97.2 | 94.7 | 99.1 | 102.3 | 104.2 | 104.0 | 106.0 |
| | (% of Day −1) | | | | | | | | | | | | | | |
| | SEM | 0.0 | 0.5 | 0.7 | 1.6 | 1.3 | 1.0 | 1.3 | 2.2 | 2.4 | 1.8 | 1.6 | 1.3 | 1.6 | 1.4 |
| 5 | Mean Body Weight | 100.0 | 100.7 | 98.4 | 105.1 | 105.4 | 108.2 | 106.5 | 107.2 | 107.0 | 106.9 | 107.5 | 109.3 | 108.3 | 108.9 |
| | (% of Day −1) | | | | | | | | | | | | | | |
| | SEM | 0.0 | 0.5 | 0.8 | 1.0 | 0.8 | 1.2 | 1.1 | 1.1 | 1.2 | 1.3 | 1.6 | 1.9 | 1.5 | 1.6 |

TABLE 41B
Mean end body weight % change from day −1.

| | Mean Body Weight | |
|---|---|---|
| Group | Change (% of Day −1) | SEM |
| Vehicle | 94.9 | 2.4 |
| Compound A (1 mg/kg) | 100.7* | 2.7 |
| Compound A (3 mg/kg) | 103.0*** | 1.8 |
| Compound A (10 mg/kg) | 106.0*** | 1.4 |
| FTY720 | 108.9*** | 1.6 |

*p < 0.05,
***p < 0.001;
statistical analyses are relative to the vehicle group (Group 1).

TABLE 41C
Mean body weight (g).

| | | Day | | | | | | | | | | | | | |
|---|---|---|---|---|---|---|---|---|---|---|---|---|---|---|---|
| Group | | −1 | 0 | 2 | 5 | 7 | 9 | 12 | 14 | 16 | 19 | 21 | 23 | 27 | 28 |
| 1 | Mean Body Weight (g) | 20.2 | 20.0 | 19.2 | 20.2 | 20.6 | 21.6 | 21.1 | 20.1 | 18.5 | 17.3 | 17.9 | 18.8 | 18.7 | 19.2 |
| | SEM | 0.3 | 0.3 | 0.3 | 0.3 | 0.3 | 0.4 | 0.5 | 0.6 | 0.6 | 0.6 | 0.5 | 0.5 | 0.6 | 0.6 |
| 2 | Mean Body Weight (g) | 20.1 | 20.3 | 19.8 | 20.0 | 20.1 | 21.4 | 20.7 | 19.9 | 19.4 | 19.6 | 20.3 | 20.7 | 20.4 | 20.4 |
| | SEM | 0.4 | 0.4 | 0.3 | 0.6 | 0.7 | 0.4 | 0.4 | 0.4 | 0.5 | 0.5 | 0.4 | 0.4 | 0.5 | 0.6 |
| 3 | Mean Body Weight (g) | 20.2 | 20.3 | 19.5 | 19.6 | 20.2 | 20.5 | 20.6 | 20.0 | 19.5 | 19.5 | 20.3 | 20.8 | 20.4 | 20.6 |
| | SEM | 0.3 | 0.3 | 0.5 | 0.6 | 0.5 | 0.4 | 0.4 | 0.4 | 0.4 | 0.4 | 0.3 | 0.2 | 0.5 | 0.4 |
| 4 | Mean Body Weight (g) | 20.2 | 20.5 | 19.8 | 20.0 | 20.4 | 20.6 | 20.2 | 19.6 | 19.1 | 20.0 | 20.7 | 21.1 | 21.0 | 21.4 |
| | SEM | 0.3 | 0.3 | 0.3 | 0.5 | 0.4 | 0.3 | 0.4 | 0.4 | 0.4 | 0.3 | 0.4 | 0.4 | 0.4 | 0.4 |
| 5 | Mean Body Weight (g) | 20.2 | 20.3 | 19.9 | 21.2 | 21.3 | 21.8 | 21.5 | 21.6 | 21.6 | 21.6 | 21.7 | 22.0 | 21.9 | 22.0 |
| | SEM | 0.3 | 0.2 | 0.2 | 0.3 | 0.3 | 0.3 | 0.3 | 0.3 | 0.3 | 0.2 | 0.2 | 0.3 | 0.3 | 0.4 |

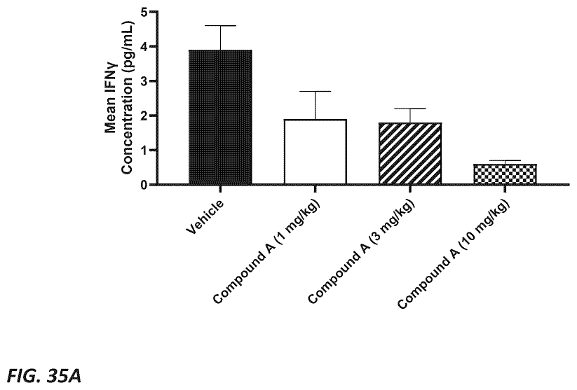

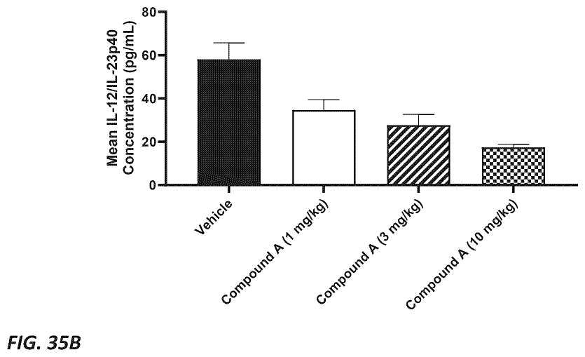

The mean concentration of cytokines collected from animals are provided in Table 42. No clear pattern was observed from the available data for cytokines IL-22, TNF, and IL-17A and nor were significant changes measured in the concentration of IL-22, TNF, and IL-17A in animals receiving Compound A relative to vehicle treated animals. Treatment with Compound A at 3 mg/kg and 10 mg/kg though resulted in a dose dependent significant decrease in IFNγ concentrations (FIG. 35A). Similarly, treatment with Compound A at all tested doses, 1 mg/kg, 3 mg/kg, and 10 mg/kg, resulted in dose dependent significant decreases in the concentration of IL-12/IL23p40 (FIG. 35B and Table 42).

TABLE 42
Cytokine concentrations.

| | Mean Concentration (pg/mL) and (SEM) | | | | |
|---|---|---|---|---|---|
| | | | | | IL-12/IL- |
| Group | IL-22 | IFNγ | TNF | IL-17A | 23p40 |
| Vehicle | 3.0 (0.9) | 3.9 (0.7) | 1.3 (0.8) | 0.1 (0.1) | 58.1 (7.6) |
| Compound | 3.5 (1.4) | 1.9 (0.8) | 0.8 (0.4) | 0.5 (0.3) | 34.7 (4.8)* |
| A (1 mg/kg) | | | | | |
| Compound | 6.1 (3.0) | 1.8 (0.4)* | 1.4 (0.4) | 1.6 (0.9) | 27.7 (4.9)** |
| A (3 mg/kg) | | | | | |
| Compound | 3.0 (0.7) | 0.6 (0.1)*** | 0.4 (0.1) | 0.4 (0.3) | 17.4 (1.5)*** |
| A (10 | | | | | |
| mg/kg) | | | | | |

*p < 0.05,
**p < 0.01,
***p < 0.0001;
statistical analyses are relative to the vehicle group (Group 1).

Without being bound by any particular theory, Compound A appears to be effective at treating EAE by reducing the severity and incidence of EAE, protecting against weight loss, and by reducing levels of certain cytokines, such as IFNγ and IL-12/IL-23p40, that play a role in autoimmune inflammatory diseases. Moreover, the somewhat bell-shape like progression of Compound A scoring curve during the course of the study from day 12 to 28 appears to indicate a therapeutic effect associated with some reversal or suppression of the disease course, which in turn may indicate that continued treatment can facilitate recovery. This culminates at Day 28 with a comparable mean EAE score for Compound A at the 10 mg/kg dose when compared to the positive control, FTY720.

Collectively, the compounds of formula I and formula IA described herein, including Compound A, demonstrate efficacy across a broad spectrum of inflammatory diseases and disorders. Compound A demonstrated a rapid onset of action, significantly reduced the clinical signs of psoriasis, markedly reduced levels of inflammatory biomarkers, and promoted clinical improvement in psoriasis that was comparable to deucravacitinib. Compound A also demonstrated superior anti-inflammatory effects on clinical signs and symptoms in arthritis relative to another BD2-selective BET inhibitor (GSK620), and suppressed the inflammatory biomarker IgG1 at levels greater than 98% of vehicle control, with the majority of animals being histologically clear of arthritis at the highest does (10 mg/kg). Compound A additionally demonstrated a marked improvement in tissue fibrosis, organ function, and in levels of biomarkers related to fibrosis in an IPF and renal fibrosis model. Compound A was also superior to pirfenidone in improving oxygen saturation in the IPF model. Further, Compound A exhibited a dose-dependent and statistically significant reduction in the inhibition of proteinuria associated with SLE. In the SLE model, animals receiving Compound A also demonstrated an improved weight gain relative to controls. Compound A also demonstrated a dose-dependent improvement in disease scores and a decrease in disease incidence in a well-characterized EAE animal model for human MS (used to study pathogenesis of autoimmunity, CNS inflammation, demyelination, cell trafficking, and tolerance induction), that was associated with protection against weight loss and a reduction in concentration of certain inflammatory cytokines. Partway through the study Compound A appeared to reverse or suppress the progression, and/or severity of the disease resulting in an end score for the 10 mg/kg group that was similar to that of FYT720. Additionally, Compound A (10 mg/kg) was observed to have no deleterious effect on gastrointestinal villus morphology or goblet cell number in healthy beagle dogs supporting its potential as an oral therapy. Collectively, these results demonstrate a broad efficacy for Compound A and the compounds of formula I and formula IA disclosed herein for treating inflammatory diseases and disorders, and diseases and disorders associated with inflammation and for treating autoimmune diseases and disorders and diseases and disorders associated with autoimmune diseases and disorders.

1.-31. (canceled)

32. A compound of formula: 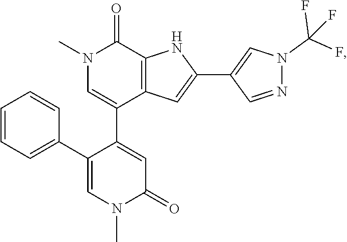 [SMILES: Cn1cc(-c2ccccc2)c(-c2cn(C)c(=O)c3[nH]c(-c4cnn(C(F)(F)F)c4)cc23)cc1=O]
or a pharmaceutically acceptable salt or N-oxide thereof.

33. A compound of formula:

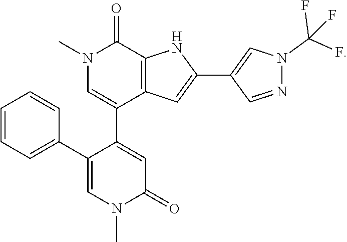 [SMILES: Cn1cc(-c2ccccc2)c(-c2cn(C)c(=O)c3[nH]c(-c4cnn(C(F)(F)F)c4)cc23)cc1=O]

34. A pharmaceutically acceptable salt of the compound of formula:

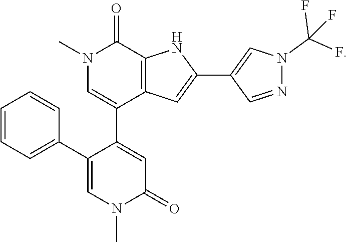 [SMILES: Cn1cc(-c2ccccc2)c(-c2cn(C)c(=O)c3[nH]c(-c4cnn(C(F)(F)F)c4)cc23)cc1=O]

35. A N-oxide of the compound of formula:

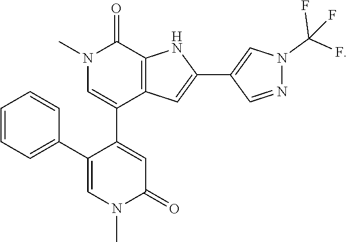 [SMILES: Cn1cc(-c2ccccc2)c(-c2cn(C)c(=O)c3[nH]c(-c4cnn(C(F)(F)F)c4)cc23)cc1=O]

36. A pharmaceutical composition comprising the compound of claim 32, or a pharmaceutically acceptable salt or N-oxide thereof, and one or more pharmaceutically acceptable excipients.

37. A pharmaceutical composition comprising the compound of claim 33, and one or more pharmaceutically acceptable excipients.

38. A pharmaceutical composition comprising the pharmaceutically acceptable salt of claim 34, and one or more pharmaceutically acceptable excipients.

39. A pharmaceutical composition comprising the N-oxide of claim 35, and one or more pharmaceutically acceptable excipients.

40. A method of treating or ameliorating an inflammatory disease or disorder, an immune disease or disorder, or an autoimmune disease or disorder, comprising administering to a subject a compound of claim 32, or a pharmaceutically acceptable salt or N-oxide thereof.

41. A method of treating or ameliorating of an inflammatory disease or disorder, an immune disease or disorder, or an autoimmune disease or disorder, comprising administering to a subject a compound of claim 33.

42. A method of treating or ameliorating of an inflammatory disease or disorder, an immune disease or disorder, or an autoimmune disease or disorder, comprising administering to a subject a pharmaceutically acceptable salt of claim 34.

43. A method of treating or ameliorating of an inflammatory disease or disorder, an immune disease or disorder, or an autoimmune disease or disorder, comprising administering to a subject a N-oxide of claim 35.

44. A method of treating or ameliorating a cancer or cancer related disease or disorder, comprising administering to a subject a compound of claim 32, or a pharmaceutically acceptable salt or N-oxide thereof.

45. A method of treating or ameliorating a cancer or cancer related disease or disorder, comprising administering to a subject a compound of claim 33.

46. A method of treating or ameliorating of a myeloproliferative neoplastic disease or disorder, comprising administering to a subject a compound of claim 32, or a pharmaceutically acceptable salt or N-oxide thereof.

47. A method of treating or ameliorating of a myeloproliferative neoplastic disease or disorder, comprising administering to a subject a compound of claim 33.

48. A method of inhibiting Bromodomain and Extra-Terminal proteins, comprising administering to a subject a compound of claim 32, or a pharmaceutically acceptable salt or N-oxide thereof.

49. A method of inhibiting Bromodomain and Extra-Terminal proteins, comprising administering to a subject a compound of claim 33.

50. A method of inhibiting Bromodomain and Extra-Terminal proteins, comprising administering to a subject a pharmaceutically acceptable salt of claim 34.

51. A method of inhibiting Bromodomain and Extra-Terminal proteins, comprising administering to a subject a N-oxide of claim 35.
